(19) United States
(12) Patent Application Publication
(10) Pub. No.: US 2020/0115406 A1
(43) Pub. Date: Apr. 16, 2020

(54) ORGANOMETALLIC COMPOUND, ORGANIC LIGHT-EMITTING DEVICE INCLUDING THE ORGANOMETALLIC COMPOUND, AND DIAGNOSTIC COMPOSITION INCLUDING THE ORGANOMETALLIC COMPOUND

(71) Applicant: Samsung Electronics Co., Ltd., Suwon-si (KR)

(72) Inventors: Jeoungin YI, Seoul (KR); Juhyun KIM, Suwon-si (KR); Sangho PARK, Anyang-si (KR); Sunyoung LEE, Seoul (KR); Seokhwan HONG, Seoul (KR); Kyuyoung HWANG, Anyang-si (KR); Yoonhyun KWAK, Seoul (KR); Sunghun LEE, Hwaseong-si (KR); Byoungki CHOI, Hwaseong-si (KR); Hyeonho CHOI, Seoul (KR)

(21) Appl. No.: 16/553,833

(22) Filed: Aug. 28, 2019

(30) Foreign Application Priority Data
Aug. 31, 2018 (KR) 10-2018-0104028
Aug. 27, 2019 (KR) 10-2019-0104976

(51) Int. Cl. C07F 15/00; H01L 51/00

(52) U.S. Cl. CPC C07F 15/0086; H01L 51/5004; H01L 51/0087

(57) ABSTRACT

An organometallic compound represented by Formula 1:

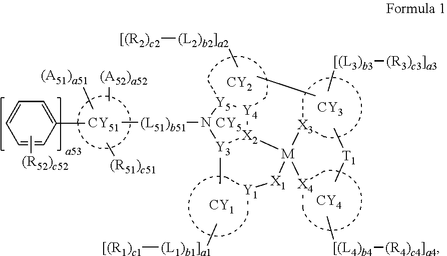

wherein, in Formula 1, groups and variables are the same as described in the specification.

### CROSS-REFERENCE TO RELATED APPLICATION

This application claims priority to Korean Patent Applications Nos. 10-2018-0104028, filed on Aug. 31, 2018, and 10-2019-0104976, filed on Aug. 27, 2019, in the Korean Intellectual Property Office, and all the benefits accruing therefrom under 35 U.S.C. § 119, the contents of which are incorporated herein in their entirety by reference.

### BACKGROUND

### 1. Field

An embodiment relates to an organometallic compound, an organic light-emitting device including the same, and a diagnostic composition including the organometallic compound.

### 2. Description of the Related Art

Organic light-emitting devices (OLEDs) are self-emission devices, which have superior characteristics in terms of a viewing angle, a response time, a brightness, a driving voltage, and a response speed, and which produce full-color images.

In an example, an organic light-emitting device includes an anode, a cathode, and an organic layer disposed between the anode and the cathode, wherein the organic layer includes an emission layer. A hole transport region may be disposed between the anode and the emission layer, and an electron transport region may be disposed between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. These excitons transit from an excited state to a ground state, thereby generating light.

Meanwhile, luminescent compounds may be used to monitor, sense, or detect a variety of biological materials including cells and proteins. An example of the luminescent compounds includes a phosphorescent luminescent compound.

Various types of organic light emitting devices are known. However, there still remains a need in OLEDs having low driving voltage, high efficiency, high brightness, and long lifespan.

### SUMMARY

Aspects of the present disclosure provide an organometallic compound, an organic light-emitting device including the same, and a diagnostic composition including the organometallic compound.

Additional aspects will be set forth in part in the description which follows and, in part, will be apparent from the description, or may be learned by practice of the presented embodiments.

An aspect of the present disclosure provides an organometallic compound represented by Formula 1:

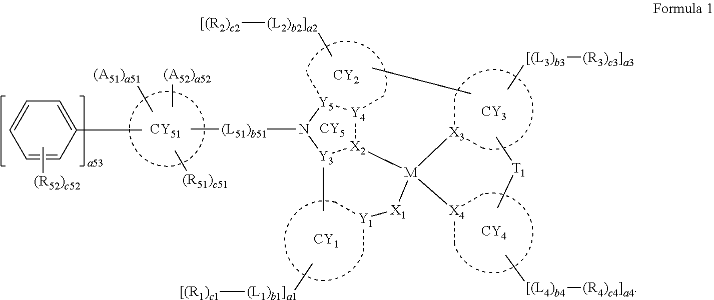

In Formula 1,
M may be a transition metal,
X₁ may be O or S, wherein a bond between X₁ and M may be a covalent bond,
X₂ to X₄ may each independently be C or N,
a bond between X₂ and M, a bond between X₃ and M, or a bond between X₄ and M may be a covalent bond, and the other bonds of a bond between X₂ and M, a bond between X₃ and M, or a bond between X₄ and M may be coordinate bonds,
Y₁ and Y₃ to Y₅ may each independently be C or N,
a bond between X₂ and Y₃, a bond between X₂ and Y₄, and a bond between Y₄ and Y₅ may be a chemical bond, ring CY₁ to ring CY₄ and ring CY₅₁ may each independently be a C₅-C₃₀ carbocyclic group or a C₂-C₃₀ heterocyclic group,
a cyclometalated ring formed by ring CY₅, ring CY₂, ring CY₃, and M may be a 6-membered ring,
T₁ may be a single bond, a double bond, *—N(R₅)—*′, *—B(R₅)—*′, *—P(R₅)—*′, *—C(R₅)(R₆)—*′, *—Si(R₅)(R₆)—*′, *—Ge(R₅)(R₆)—*′, *—S—*′, *—Se—*′, *—O—*′, *—C(═O)—*′, *—S(═O)—*′, *—S(═O)₂—*′, *—C(R₅)=*′, *═C(R₅)—*′, *—C(R₅)═C(R₆)—*′, *—C(═S)—*′, or *—C≡C—*′, wherein * and *′ each indicate a binding site to a neighboring atom,
L₁ to L₄ and L₅₁ may each independently be a single bond, a C₅-C₃₀ carbocyclic group that is unsubstituted or substituted with an R₁₀ₐ group, or a C₂-C₃₀ heterocyclic group that is unsubstituted or substituted with an R₁₀ₐ group,
b1 to b4 and b51 may each independently be an integer from 1 to 5,
R₁ to R₆, R₅₁, and R₅₂ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₇-C₆₀ arylalkyl group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted C₂-C₆₀ heteroarylalkyl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉),
c1 to c4, c51, and c52 may each independently be an integer from 1 to 5,
A₅₁ may be a C₄-C₆₀ alkyl group,
A₅₂ may be deuterium or a deuterium-containing C₁-C₆₀ alkyl group unsubstituted or substituted with a C₃-C₁₀ cycloalkyl group,
a1 to a4, a51, and a52 may each independently be an integer from 0 to 10, provided that the sum of a51 and a52 may be 1 or more,
a53 may be an integer from 1 to 10,
two or more groups of a plurality of R₁ groups may optionally be linked to form a C₅-C₃₀ carbocyclic group that is unsubstituted or substituted with an R₁₀ₐ group or a C₂-C₃₀ heterocyclic group that is unsubstituted or substituted with an R₁₀ₐ group,
two or more groups of a plurality of R₂ groups may optionally be linked to form a C₅-C₃₀ carbocyclic group that is unsubstituted or substituted with an R₁₀ₐ group or a C₂-C₃₀ heterocyclic group that is unsubstituted or substituted with an R₁₀ₐ group,
two or more groups of a plurality of R₃ groups may optionally be linked to form a C₅-C₃₀ carbocyclic group that is unsubstituted or substituted with an R₁₀ₐ group or a C₂-C₃₀ heterocyclic group that is unsubstituted or substituted with an R₁₀ₐ group,
two or more groups of a plurality of R₄ groups may optionally be linked to form a C₅-C₃₀ carbocyclic group that is unsubstituted or substituted with an R₁₀ₐ group or a C₂-C₃₀ heterocyclic group that is unsubstituted or substituted with an R₁₀ₐ group,
two or more groups of R₁ to R₆ may optionally be linked to form a C₅-C₃₀ carbocyclic group that is unsubstituted or substituted with an R₁₀ₐ group or a C₂-C₃₀ heterocyclic group that is unsubstituted or substituted with an R₁₀ₐ group,
R₁₀ₐ may be the same as described in connection with R₁,
a substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₇-C₆₀ arylalkyl group, the substituted C₁-C₆₀ heteroaryl group, the substituted C₇-C₆₀ arylalkyl group, the substituted C₁-C₆₀ heteroaryl group, the substituted C₁-C₆₀ heteroaryloxy group, the substituted C₁-C₆₀ heteroarylthio group, the substituted C₂-C₆₀ heteroarylalkyl group, the substituted monovalent non-aromatic condensed polycyclic group, and the substituted monovalent non-aromatic condensed heteropolycyclic group is:
a deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or any combination thereof;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, and a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or any combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or
any combination thereof; and
Q₁ to Q₉, Q₁₁ to Q₁₉, Q₂₁ to Q₂₉, and Q₃₁ to Q₃₉ are each independently hydrogen; deuterium; —F; —Cl; —Br; —I; a hydroxyl group; a cyano group; a nitro group; an amidino group; a hydrazine group; a hydrazone group; a carboxylic acid group or a salt thereof; a sulfonic acid group or a salt thereof; a phosphoric acid group or a salt thereof; a C₁-C₆₀ alkyl group unsubstituted or substituted with deuterium, a C₁-C₆₀ alkyl group, a C₆-C₆₀ aryl group, or any combination thereof; a C₂-C₆₀ alkenyl group; a C₂-C₆₀ alkynyl group; a C₁-C₆₀ alkoxy group; a C₃-C₁₀ cycloalkyl group; a C₂-C₁₀ heterocycloalkyl group; a C₃-C₁₀ cycloalkenyl group; a C₂-C₁₀ heterocycloalkenyl group; a C₆-C₆₀ aryl group unsubstituted or substituted with deuterium, a C₁-C₆₀ alkyl group, a C₆-C₆₀ aryl group, or any combination thereof; a C₆-C₆₀ aryloxy group; a C₆-C₆₀ arylthio group; a C₇-C₆₀ arylalkyl group; a C₁-C₆₀ heteroaryl group; a C₁-C₆₀ heteroaryloxy group; a C₁-C₆₀ heteroarylthio group; a C₂-C₆₀ heteroarylalkyl group; a monovalent non-aromatic condensed polycyclic group; or a monovalent non-aromatic condensed heteropolycyclic group.

Another aspect of the present disclosure provides an organic light-emitting device including:
a first electrode;
a second electrode; and
an organic layer disposed between the first electrode and the second electrode,
wherein the organic layer includes an emission layer and an organometallic compound represented by Formula 1.

The organometallic compound included in the emission layer in the organic layer may act as a dopant.

Another aspect of the present disclosure provides a diagnostic composition including an organometallic compound represented by Formula 1.

### BRIEF DESCRIPTION OF THE DRAWING

These and/or other aspects will become apparent and more readily appreciated from the following description of the embodiments, taken in conjunction with the FIGURE which is a schematic view of an organic light-emitting device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

In an embodiment, an organometallic compound is provided. The organometallic compound according to an embodiment may be represented by Formula 1:

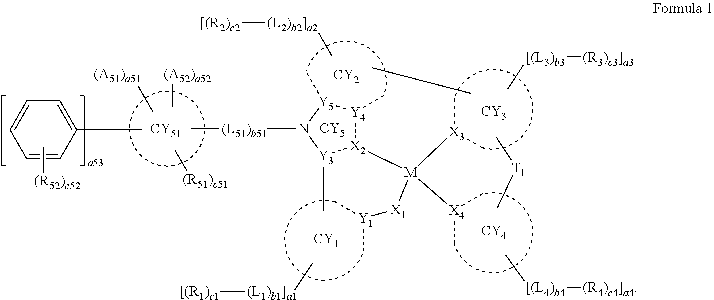

In Formula 1, M may be a transition metal. Alternatively, M may be beryllium (Be), magnesium (Mg), aluminum (Al), calcium (Ca), titanium (Ti), manganese (Mn), cobalt (Co), copper (Cu), zinc (Zn), gallium (Ga), germanium (Ge), zirconium (Zr), ruthenium (Ru), rhodium (Rh), palladium (Pd), silver (Ag), rhenium (Re), platinum (Pt), or gold (Au). For example, M may be Pt, Pd, or Au.

In Formula 1, X₁ may be O or S, wherein a bond between X₁ and M may be a covalent bond. For example, X₁ may be O.

In Formula 1, X₂ to X₄ may each independently be C or N. For example, X₂ and X₃ may each independently be C or N, and X₄ may be N.

In an embodiment, in Formula 1, X₂ and X₄ may each be N, and X₃ may be C, but embodiments of the present disclosure are not limited thereto.

In Formula 1, a bond between X₂ and M, a bond between X₃ and M, or a bond between X₄ and M may be a covalent bond, and the other bonds of a bond between X₂ and M, a bond between X₃ and M, and a bond between X₄ and M may be coordinate bonds. Therefore, the organometallic compound represented by Formula 1 may be electrically neutral.

In an embodiment, a bond between X₂ and M and a bond between X₄ and M may be a coordinate bond, and a bond between X₃ and M may be a covalent bond, but embodiments of the present disclosure are not limited thereto.

In Formula 1, Y₁ and Y₃ to Y₅ may each independently be C or N. For example, Y₁ and Y₃ to Y₅ may be C.

In Formula 1, a bond between X₂ and Y₃, a bond between X₂ and Y₄, and a bond between Y₄ and Y₅ may be a chemical bond (for example, a single bond, a double bond, or the like).

In Formula 1, ring CY₁ to ring CY₄ and ring CY₅₁ may each independently be a C₅-C₃₀ carbocyclic group or a C₂-C₃₀ heterocyclic group.

For example, ring CY₁ to ring CY₄ and ring CY₅₁ may each independently be i) a first ring, ii) a second ring, iii) a condensed ring in which at least two first rings are condensed, iv) a condensed ring in which at least two second rings are condensed, or v) a condensed ring in which a first ring and a second ring are condensed.

In an embodiment, the first ring may be a cyclopentane group, a cyclopentadiene group, a furan group, a thiophene group, a pyrrole group, a silole group, an indene group, a benzofuran group, a benzothiophene group, an indole group, a benzosilole group, an oxazole group, an isoxazole group, an oxadiazole group, an isoxadiazole group, an oxatriazole group, an isoxatriazole group, a thiazole group, an isothiazole group, a thiadiazole group, an isothiadiazole group, a thiatriazole group, an isothiatriazole group, a pyrazole group, an imidazole group, a triazole group, a tetrazole group, an azasilole group, a diazasilole group, or a triazasilole group, and the second ring may be an adamantane group, a norbornene group, a bicyclo[1.1.1]pentane group, a bicyclo[2.1.1]hexane group, a bicyclo[2.2.1]heptane group, a bicyclo[2.2.2]octane group, a cyclohexane group, a cyclohexene group, a benzene group, a pyridine group, a pyrimidine group, a pyrazine group, a pyridazine group, or a triazine group.

In an embodiment, in Formula 1, ring CY₁ to ring CY₄ and ring CY₅₁ may each independently be a cyclopentane group, a cyclohexane group, a cycloheptane group, a cyclopentene group, a cyclohexene group, a cycloheptene group, a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group.

In an embodiment, in Formula 1, ring CY₄ may not be a benzimidazole group.

In Formula 1, a cyclometalated ring formed by ring CY₅, ring CY₂, ring CY₃, and M may be a 6-membered ring.

In Formula 1, T₁ may be a single bond, a double bond, *—N(R₅)—*′, *—B(R₅)—*′, *—P(R₅)—*′, *—C(R₅)(R₆)—*′, *—Si(R₅)(R₆)—*′, *—Ge(R₅)(R₆)—*′, *—S—*′, *—Se—*′, *—O—*′, *—C(═O)—*′, *—S(═O)—*′, *—S(═O)₂—*′, *—C(R₅)=*′, *═C(R₅)—*′, *—C(R₅)═C(R₆)—*′, *—C(═S)-, or *—C≡C—*′, wherein * and *′ each indicate a binding site to a neighboring atom. Here, R₅ and R₆ are the same as described above, and may optionally be linked to each other via a single bond, a double bond, *—N(R′)—*′, *—B(R′)—*′, *—P(R′)—*′, *—C(R′)(R″)—*′, *—Si(R′)(R″)—*′, *—Ge(R′)(R″)—*′, *—S—*′, *—Se—*′, *—O—*′, *—C(═O)—*′, *—S(═O)—*′, *—S(═O)₂—*′, *—C(R′)=*′ *═C(R′)—*′, *—C(R′)═C(R″)—*′, *—C(═S)—*′, or *—C≡C—*′ to form a C₅-C₃₀ carbocyclic group that is unsubstituted or substituted with an R₁₀ₐ group or a C₂-C₃₀ heterocyclic group that is unsubstituted or substituted with an R₁₀ₐ group. R′, R″, and R₁₀ₐ are each independently defined the same as R₁.

For example, in Formula 1, T₁ may be a single bond, but embodiments of the present disclosure are not limited thereto.

In Formula 1, L₁ to L₄ and L₅₁ may each independently be a single bond, a C₅-C₃₀ carbocyclic group that is unsubstituted or substituted with an R₁₀ₐ group, or a C₂-C₃₀ heterocyclic group that is unsubstituted or substituted with an R₁₀ₐ group. Here, R₁₀ₐ is defined the same as R₁.

For example, in Formula 1, L₁ to L₄ and L₅₁ may each independently be:
a single bond; or
a benzene group, a naphthalene group, an anthracene group, a phenanthrene group, a triphenylene group, a pyrene group, a chrysene group, a cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an azafluorene group, an azacarbazole group, an azadibenzofuran group, an azadibenzothiophene group, an azadibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, or a benzothiadiazole group, each unsubstituted or substituted with an R₁₀ₐ group.

In an embodiment, in Formula 1, L₁ to L₄ and L₅₁ may each independently be:
a single bond; or
a benzene group unsubstituted or substituted with an R₁₀ₐ group,
but embodiments of the present disclosure are not limited thereto.

In an embodiment, in Formula 1,

Y₁ and X₃ may be C, and X₂ and X₄ may be N,

ring CY₁ to ring CY₃ and ring CY₅₁ may be a benzene group, and ring CY₄ may be a pyridine group, and

L₄ and L₅₁ may each independently be:
a single bond; or
a benzene group unsubstituted or substituted with an R₁₀ₐ group.

In Formula 1, b1 to b4 and b51 each indicate the number of L₁ to L₄ and L₅₁, respectively, and may each independently be an integer from 1 to 5. When b1 is two or more, two or more L₁ groups may be identical to or different from each other, when b2 is two or more, two or more L₂ groups may be identical to or different from each other, when b3 is two or more, two or more L₃ groups may be identical to or different from each other, when b4 is two or more, two or more L₄ groups may be identical to or different from each other, and when b51 is two or more, two or more L₅₁ groups may be identical to or different from each other.

For example, in Formula 1, b1 to b4 and b51 may each independently be 1, 2 or 3, but embodiments of the present disclosure are not limited thereto.

In Formula 1, R₁ to R₆, R₅₁, and R₅₂ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₇-C₆₀ arylalkyl group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted C₂-C₆₀ heteroarylalkyl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉), wherein Q₁ to Q₉ are each independently the same as described above.

For example, R₁ to R₆, R₅₁, and R₅₂ may each independently be:
a hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, C₁-C₂₀ alkyl group, or a C₁-C₂₀ alkoxy group;
a C₁-C₂₀ alkyl group and a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantyl (adamantyl) group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a pyridinyl group, a pyrimidinyl group, or any combination thereof;
a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, or an azadibenzothiophenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a deuterium-containing C₂-C₂₀ alkyl group (for example, *—C(CD₃)₃), a C₁-C₂₀ alkoxy group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, or any combination thereof; or
—N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉), and
Q₁ to Q₉ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or
an n-propyl group, an iso-propyl group, an n-butyl group, a sec-butyl group, an iso-butyl group, a tert-butyl group, an n-pentyl group, a tert-pentyl group, a neo-pentyl group, an iso-pentyl group, a sec-pentyl group, a 3-pentyl group, a sec-iso-pentyl group, a phenyl group, a biphenyl group or a naphthyl group, each unsubstituted or substituted with deuterium, a C₁-C₁₀ alkyl group, a phenyl group, or any combination thereof,
but embodiments of the present disclosure are not limited thereto.

In Formula 1, c1 to c4, c51, and c52 each indicate the number of R₁ to R₄, R₅₁, and R₅₂, respectively, and may each independently be an integer from 1 to 5. When c1 is two or more, two or more R₁ groups may be identical to or different from each other, when c2 is two or more, two or more R₂ groups may be identical to or different from each other, when c3 is two or more, two or more R₃ groups may be identical to or different from each other, when c4 is two or more, two or more R₄ groups may be identical to or different from each other, when c51 is two or more, two or more R₅₁ groups may be identical to or different from each other, and when c52 is two or more, two or more R₅₂ groups may be identical to or different from each other. For example, c1 to c4, c51, and c52 may each independently be 1 or 2, but embodiments of the present disclosure are not limited thereto.

In Formula 1, A₅₁ may be a C₄-C₆₀ alkyl group, and A₅₂ may be deuterium or a deuterium-containing C₁-C₆₀ alkyl group unsubstituted or substituted with a C₃-C₁₀ cycloalkyl group.

In an embodiment, in Formula 1, A₅₁ may be a linear or branched C₄-C₁₀ alkyl group, and A₅₂ may be deuterium or a deuterium-containing linear or branched C₁-C₂₀ alkyl group unsubstituted or substituted with a C₃-C₁₀ cycloalkyl group.

In an embodiment, in Formula 1, A₅₁ may be an n-butyl group, a sec-butyl group, an iso-butyl group, a tert-butyl group, an n-pentyl group, a tert-pentyl group, a neo-pentyl group, an iso-pentyl group, a sec-pentyl group, a 3-pentyl group, or a sec-iso-pentyl group, unsubstituted or substituted with a methyl group, an ethyl group, an n-propyl group, an iso-propyl group, an n-butyl group, a sec-butyl group, an iso-butyl group, a tert-butyl group, an n-pentyl group, a tert-pentyl group, a neo-pentyl group, an iso-pentyl group, a sec-pentyl group, a 3-pentyl group, a sec-iso-pentyl group, or any combination thereof. For example, Formula 9-33 may be a branched C₆ alkyl group and a tert-butyl group substituted with two methyl groups.

In an embodiment, in Formula 1, A₅₂ may be a deuterium-containing linear or branched C₁-C₂₀ alkyl group unsubstituted or substituted with a C₃-C₁₀ cycloalkyl group, in which the linear or branched C₁-C₂₀ alkyl group may be a methyl group, an ethyl group, an n-propyl group, an iso-propyl group, an n-butyl group, a sec-butyl group, an iso-butyl group, a tert-butyl group, an n-pentyl group, a tert-pentyl group, a neo-pentyl group, an iso-pentyl group, a sec-pentyl group, a 3-pentyl group, or a sec-iso-pentyl group, unsubstituted or substituted with a methyl group, an ethyl group, an n-propyl group, an iso-propyl group, an n-butyl group, a sec-butyl group, an iso-butyl group, a tert-butyl group, an n-pentyl group, a tert-pentyl group, a neo-pentyl group, an iso-pentyl group, a sec-pentyl group, a 3-pentyl group, a sec-iso-pentyl group, or any combination thereof.

The deuterium-containing C₁-C₆₀ alkyl group (or, the deuterium-containing C₁-C₂₀ alkyl group, the deuterium-containing C₂-C₂₀ alkyl group, etc.) means a C₁-C₆₀ alkyl group substituted with a deuterium (or, a C₁-C₂₀ alkyl group substituted with a deuterium, a C₂-C₂₀ alkyl group substituted with a deuterium, etc.). For example, a deuterium-containing C₁ alkyl group (that is, a deuterium-containing methyl group) includes —CD₃, —CD₂H and —CDH₂.

The deuterium-containing C₁-C₆₀ alkyl group (or, the deuterium-containing C₁-C₂₀ alkyl group, the deuterium-containing C₂-C₂₀ alkyl group, etc.) may be additionally substituted with a C₃-C₁₀ cycloalkyl group. For example, Formula 9-619 is a group corresponding to —CD₂H, in which “—H” is substituted with a cyclopentyl group.

The organometallic compound represented by Formula 1 may include a deuterium, a deuterium-containing C₁-C₆₀ alkyl group unsubstituted or substituted with a C₃-C₁₀ cycloalkyl group, or any combination thereof. The number of deuterium atoms in the organometallic compound represented by Formula 1 may be from 1 to 20, for example, from 1 to 15, from 1 to 10, or from 1 to 5.

In an embodiment, regarding Formula 1,
R₁ to R₆, R₅₁, and R₅₂ may each independently be:
hydrogen, deuterium, —F, a cyano group, a nitro group, —SF₅, —CH₃, —CD₃, —CD₂H, —CDH₂, —CF₃, —CF₂H, —CFH₂, a group represented by one of Formulae 9-1 to 9-66, a group represented by one of Formulae 9-1 to 9-66 in which a hydrogen is substituted with deuterium, a group represented by one of Formulae 10-1 to 10-118, a group represented by one of Formulae 10-1 to 10-118 in which a hydrogen is substituted with deuterium, a group represented by one of Formulae 10-201 to 10-342, or a group represented by one of Formulae 10-201 to 10-342 in which a hydrogen is substituted with deuterium, and/or
A₅₁ may be a group represented by one of Formulae 9-4 to 9-36, and/or
A₅₂ may be a group represented by one of Formulae 9-1 to 9-63 in which a hydrogen is substituted with deuterium, and/or
a group represented by

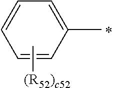

in Formula 1 may be a group represented by one of Formulae 10-10 to 10-118, or a group represented by one of Formulae 10-10 to 10-118 in which a hydrogen is substituted with deuterium:

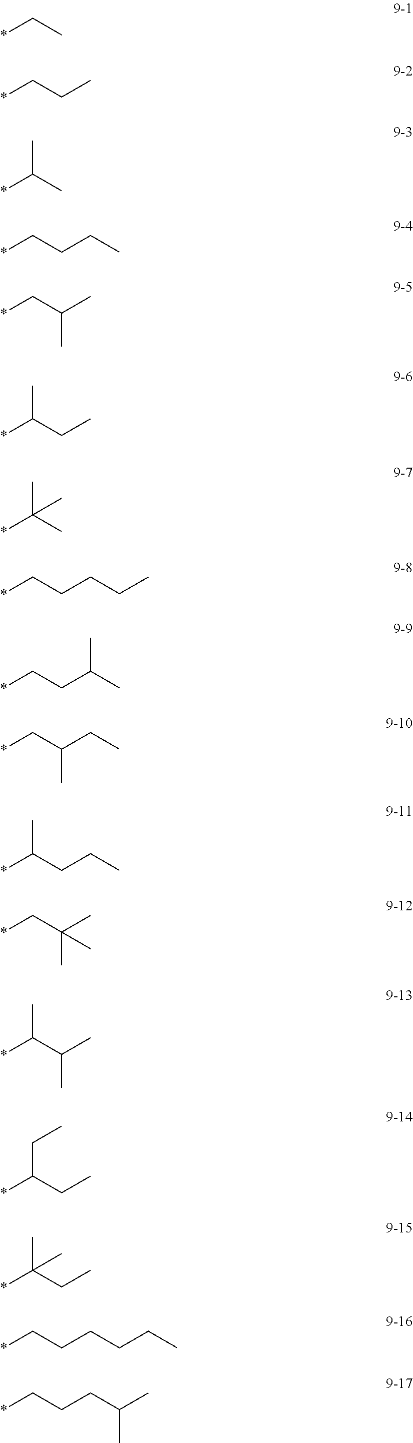

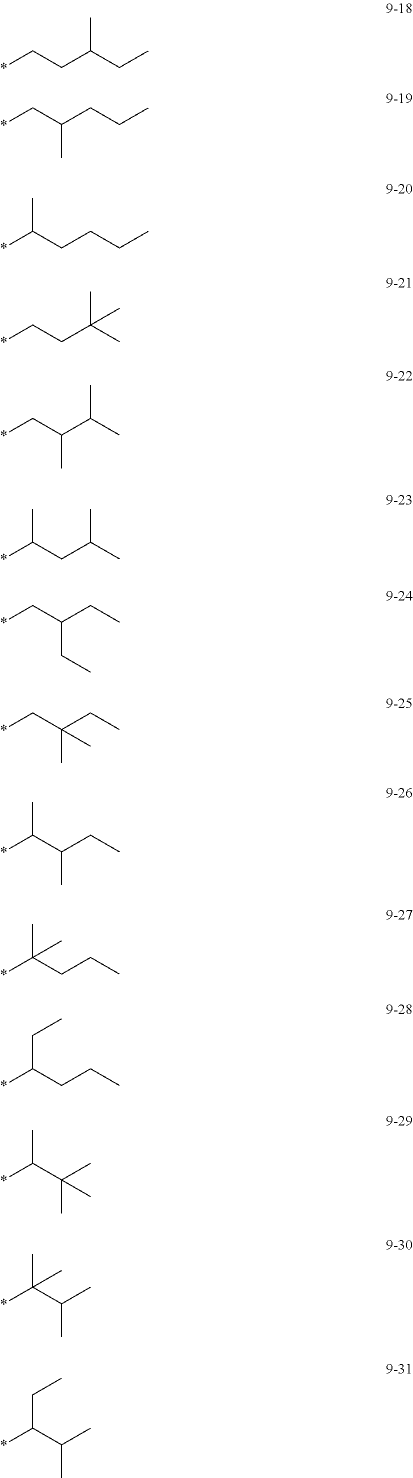

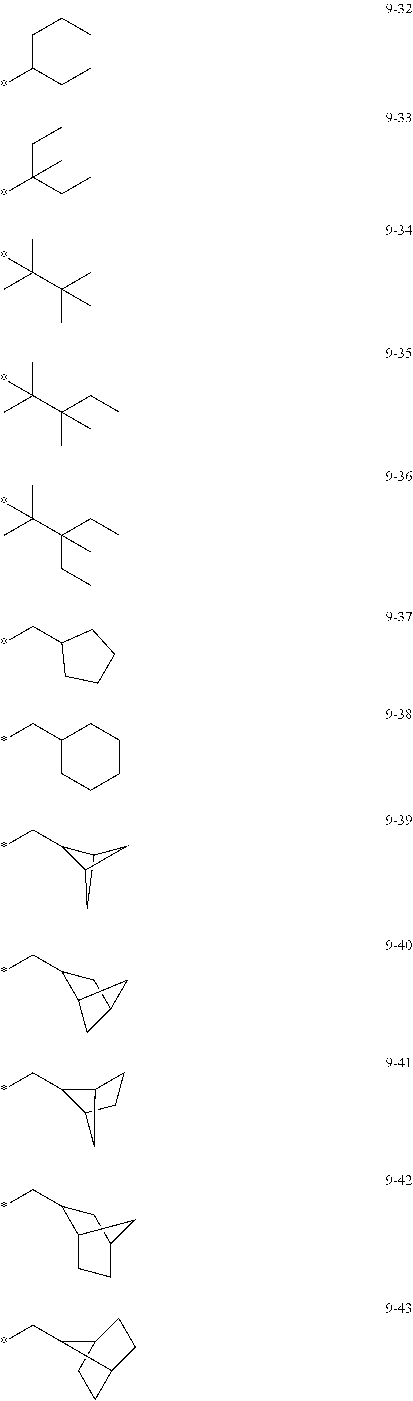

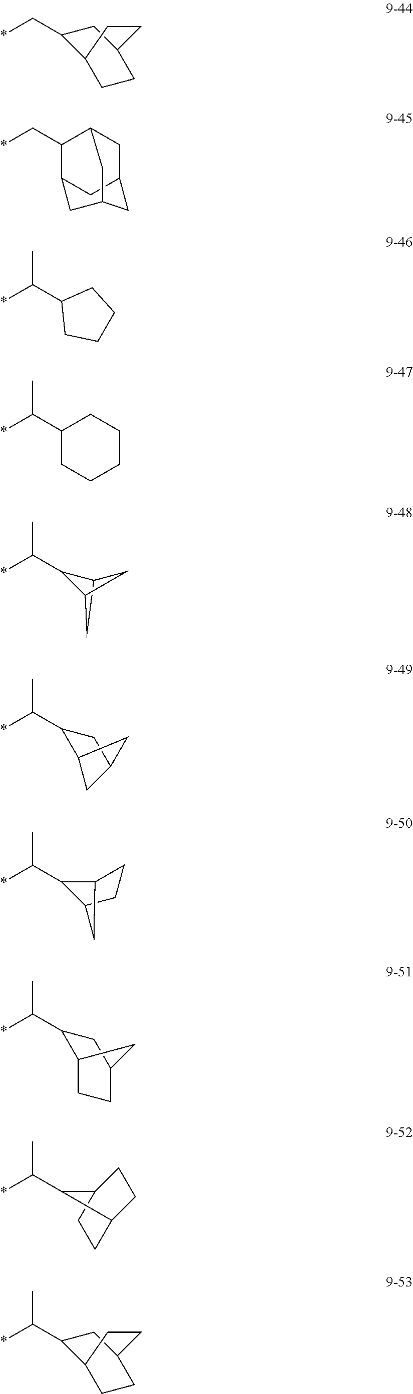

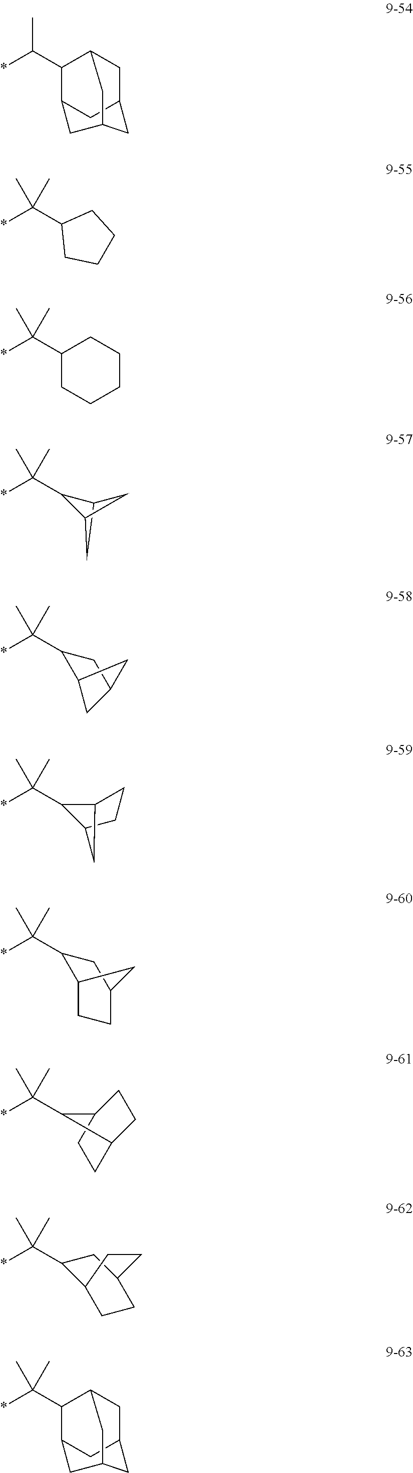

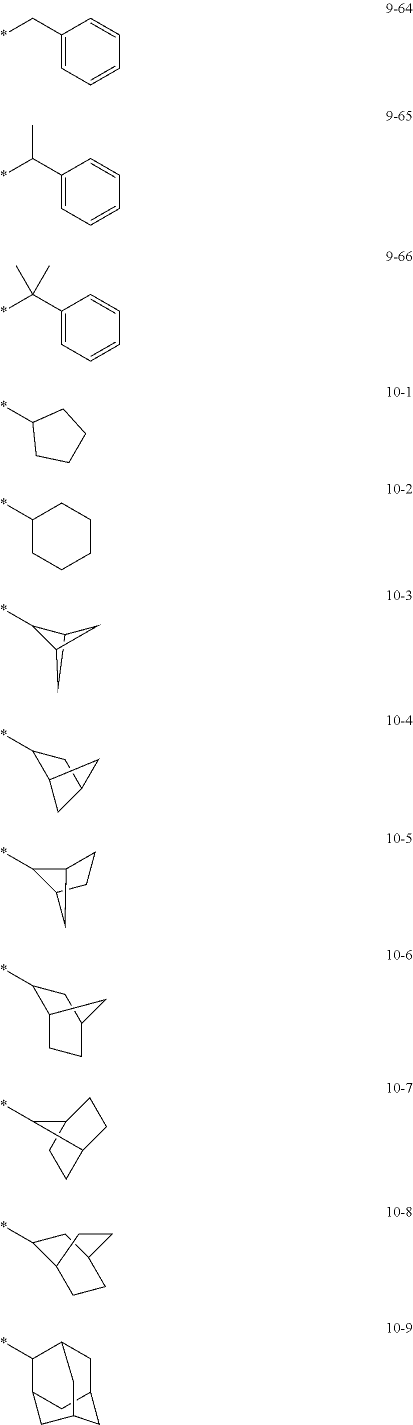

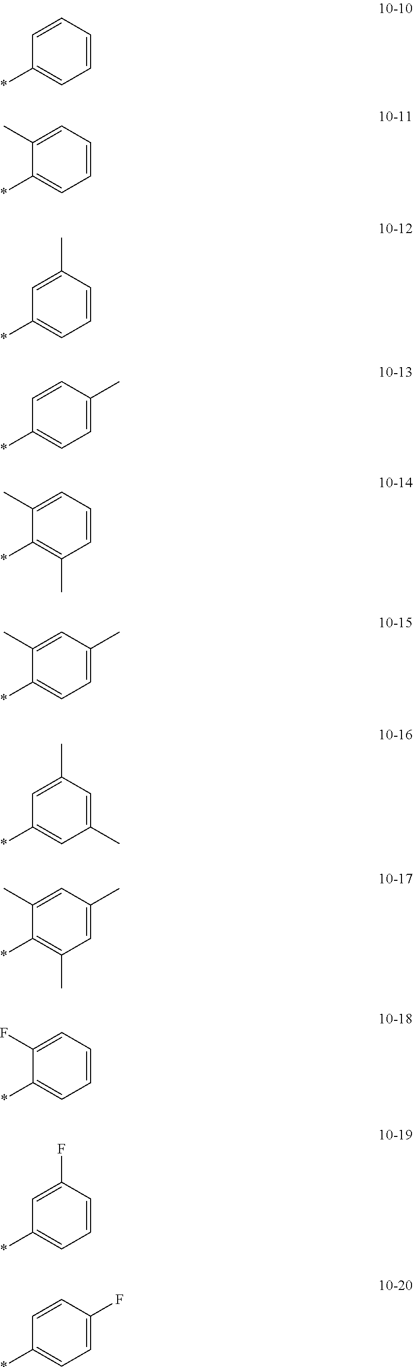

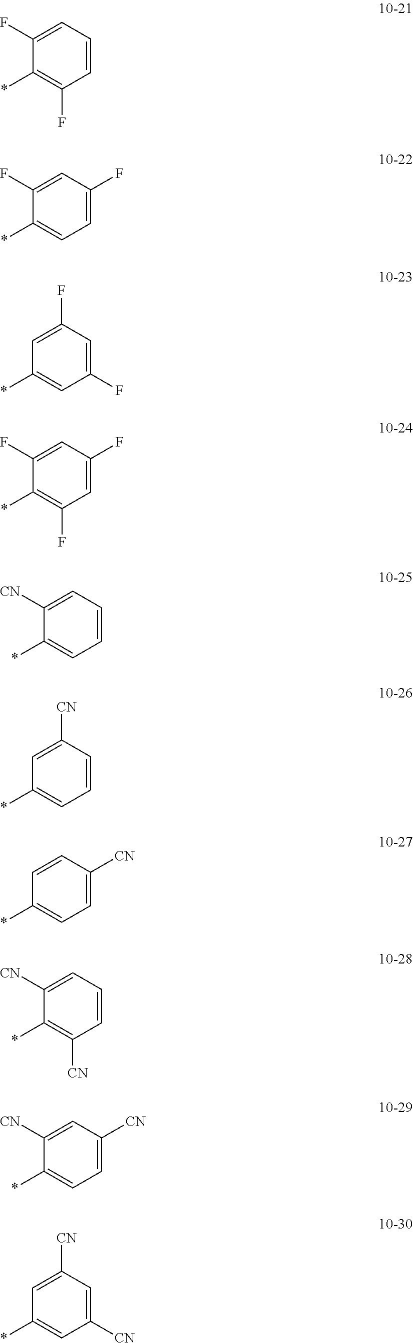

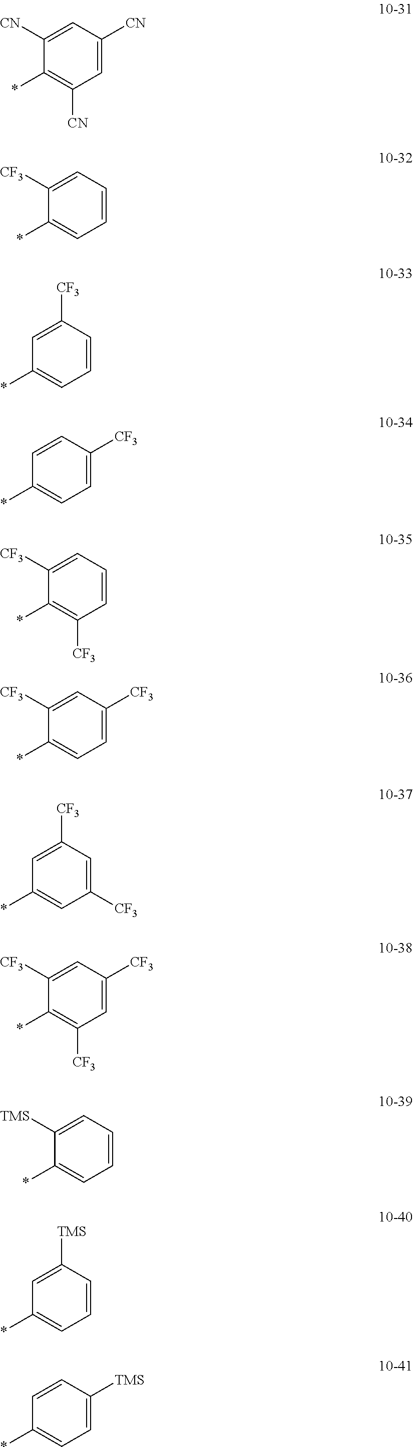

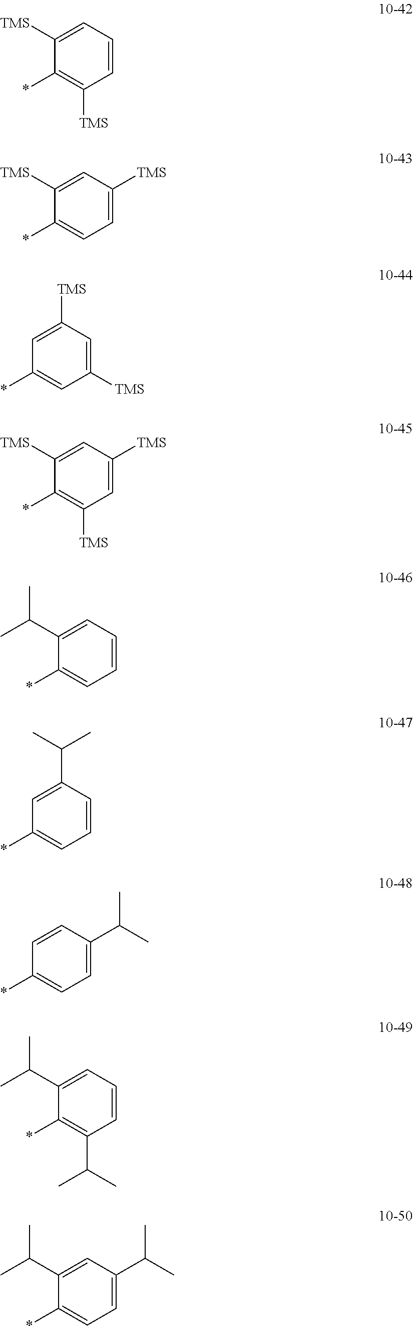

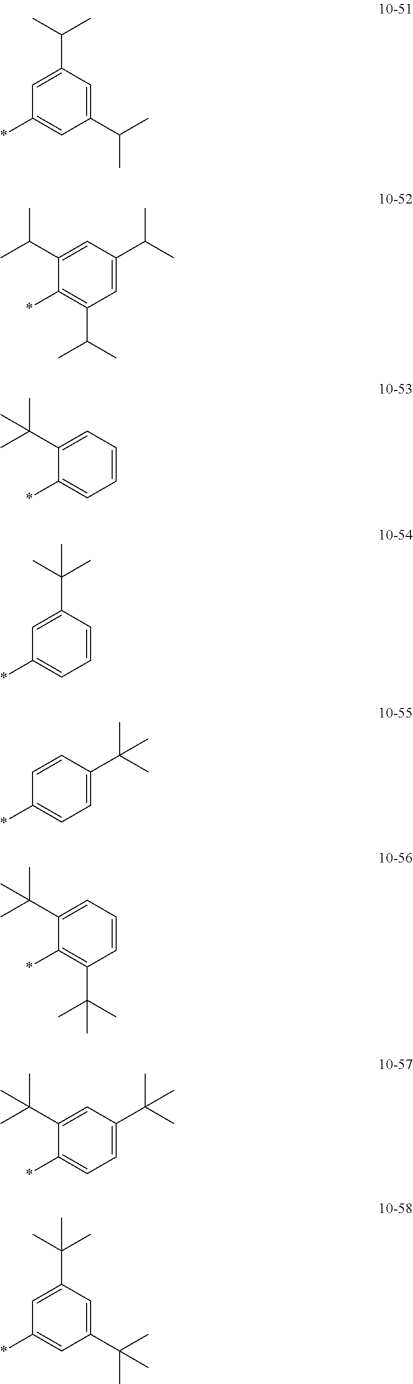

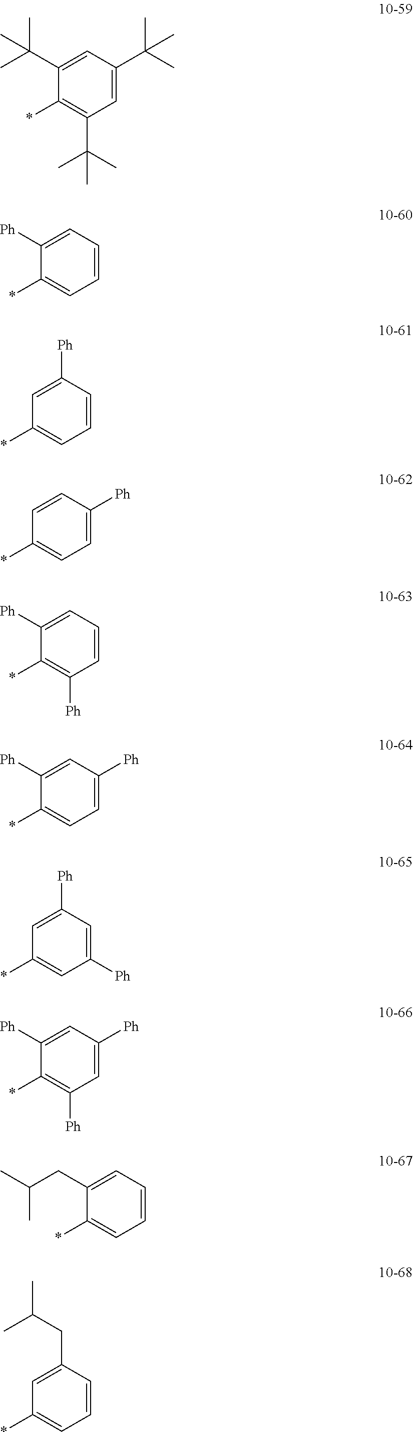

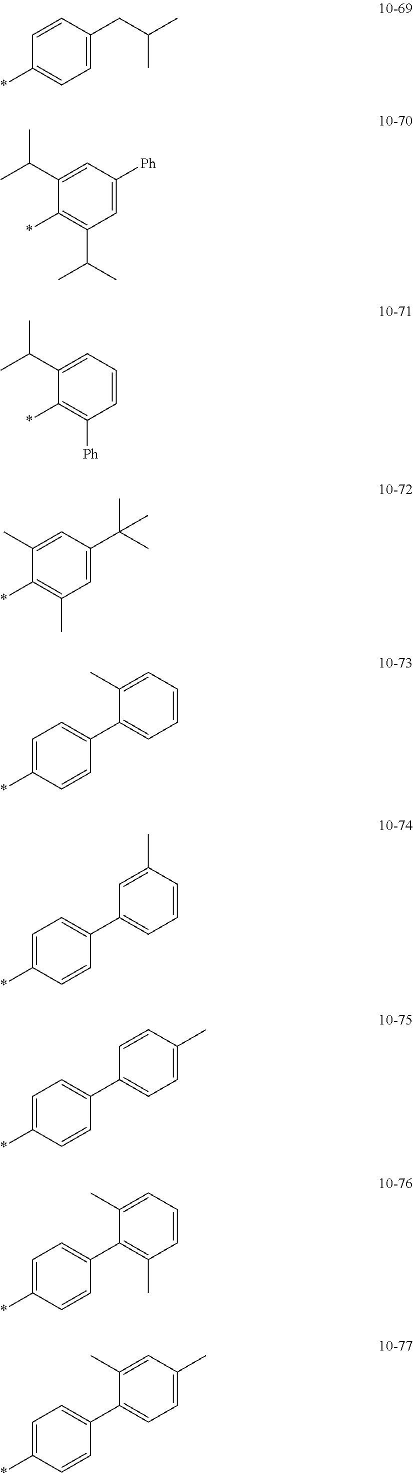

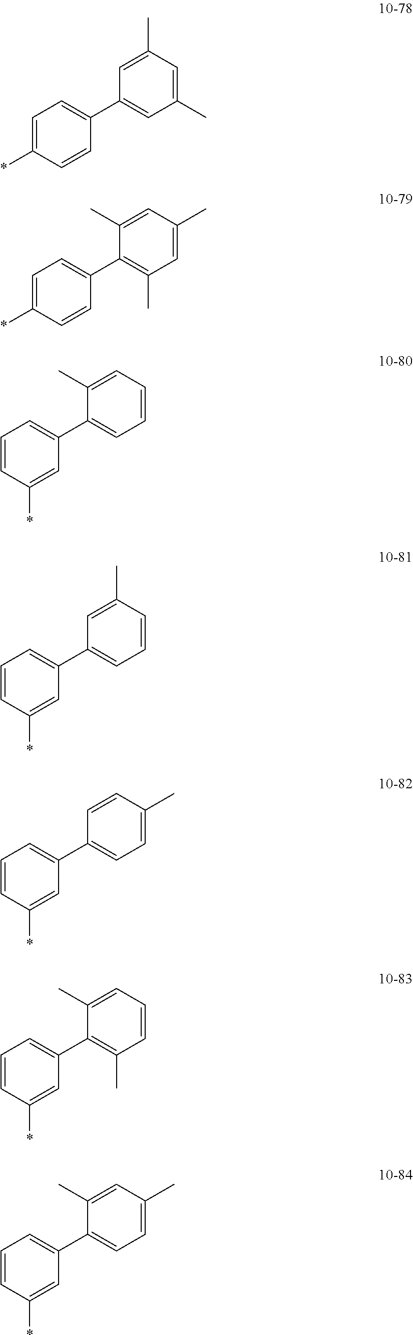

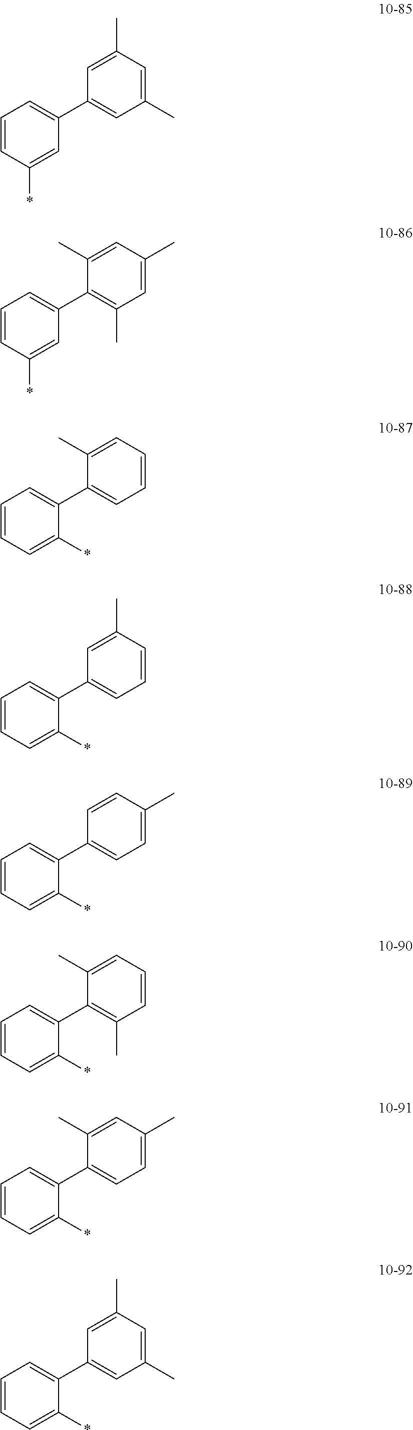

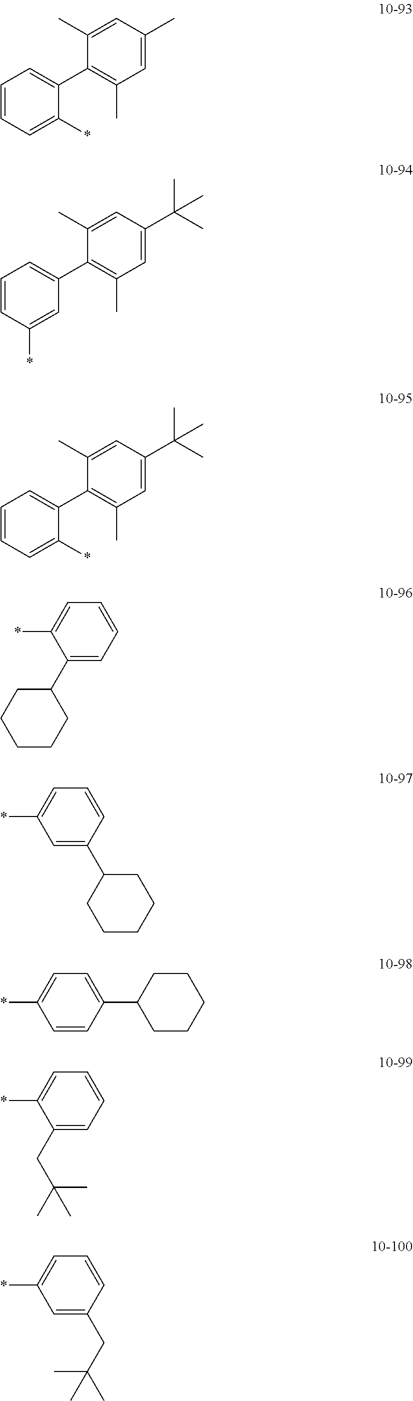

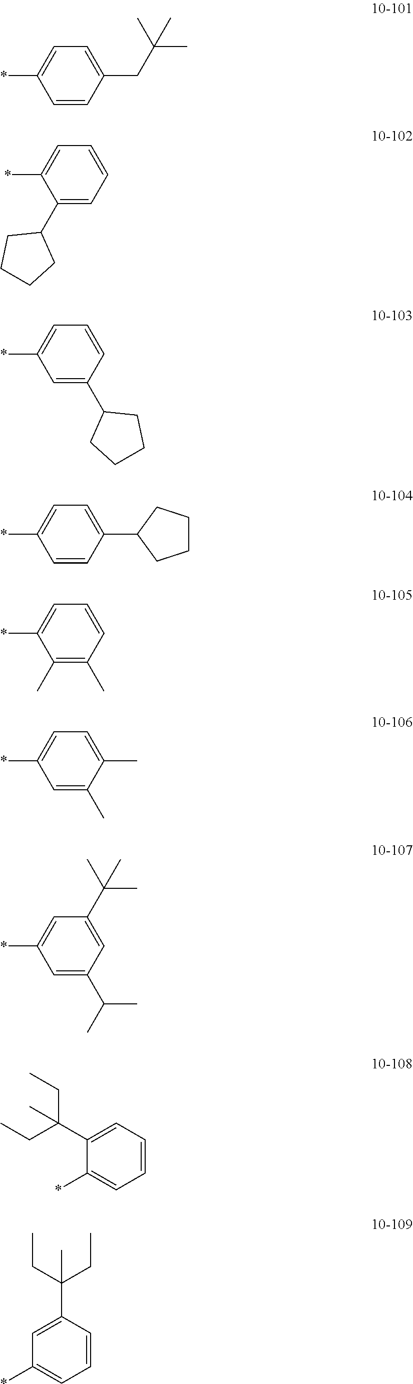

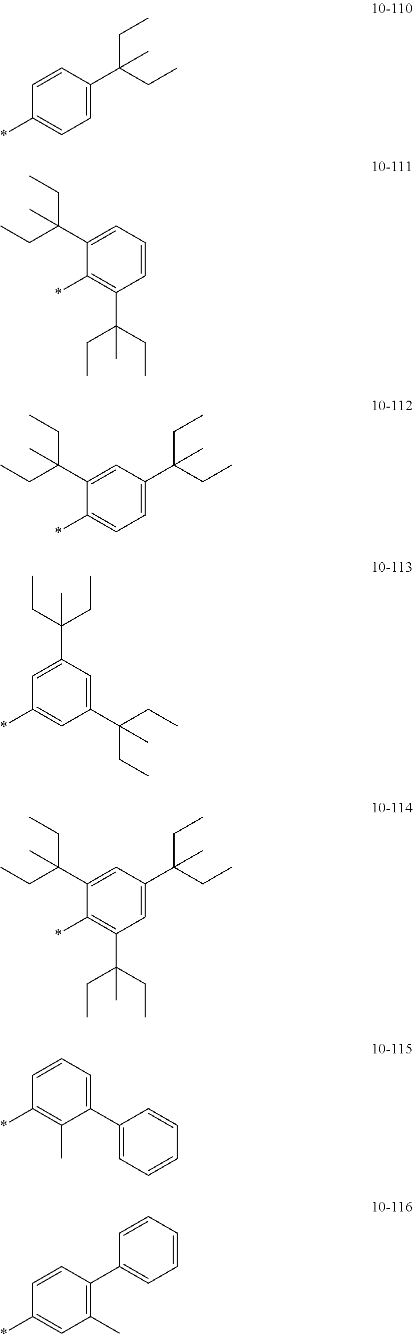

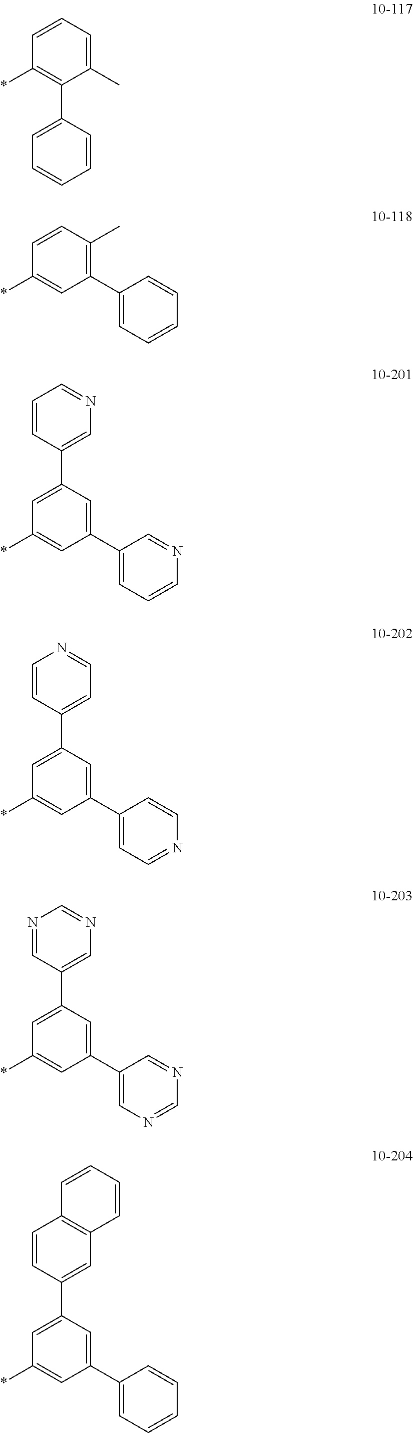

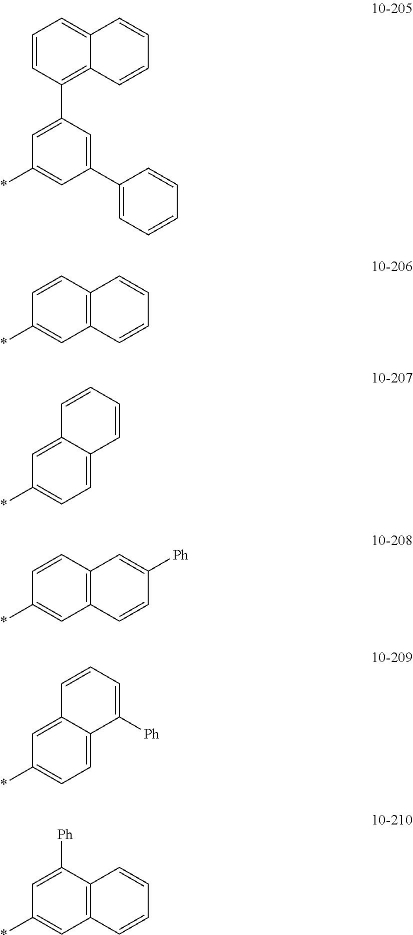 [SMILES: *c1cc(-c2ccccc2)cc(-c2cccc3ccccc23)c1 ; *c1ccc2ccccc2c1 ; *c1ccc2ccccc2c1 ; *c1ccc2cc(-c3ccccc3)ccc2c1 ; *c1ccc2c(-c3ccccc3)cccc2c1 ; *c1cc(-c2ccccc2)c2ccccc2c1]

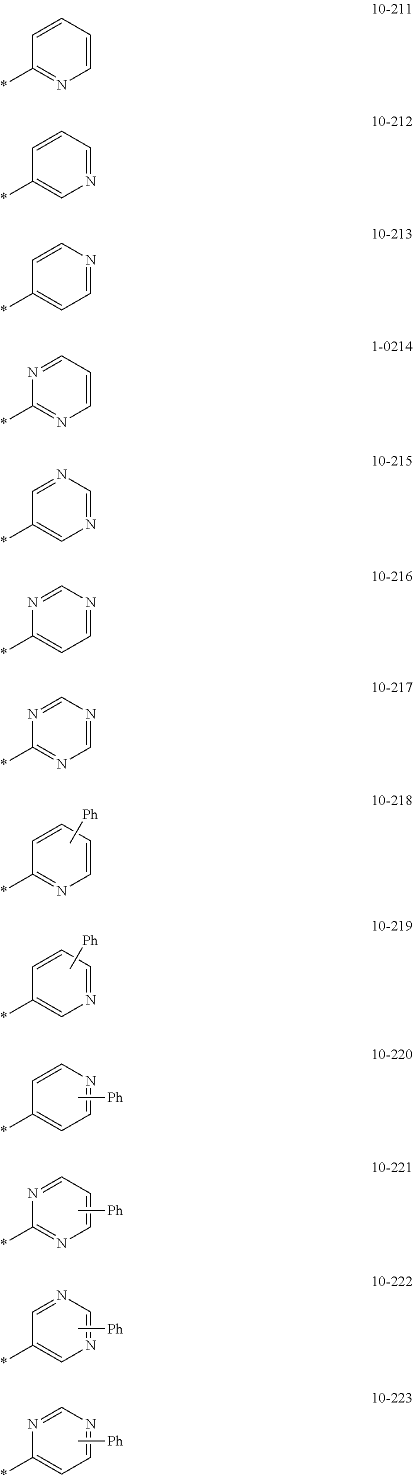

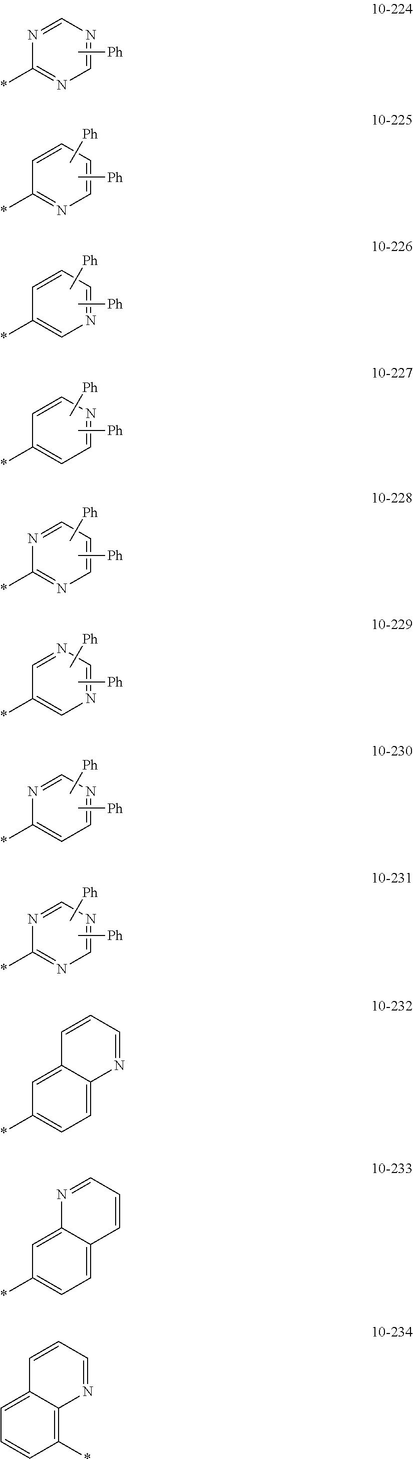

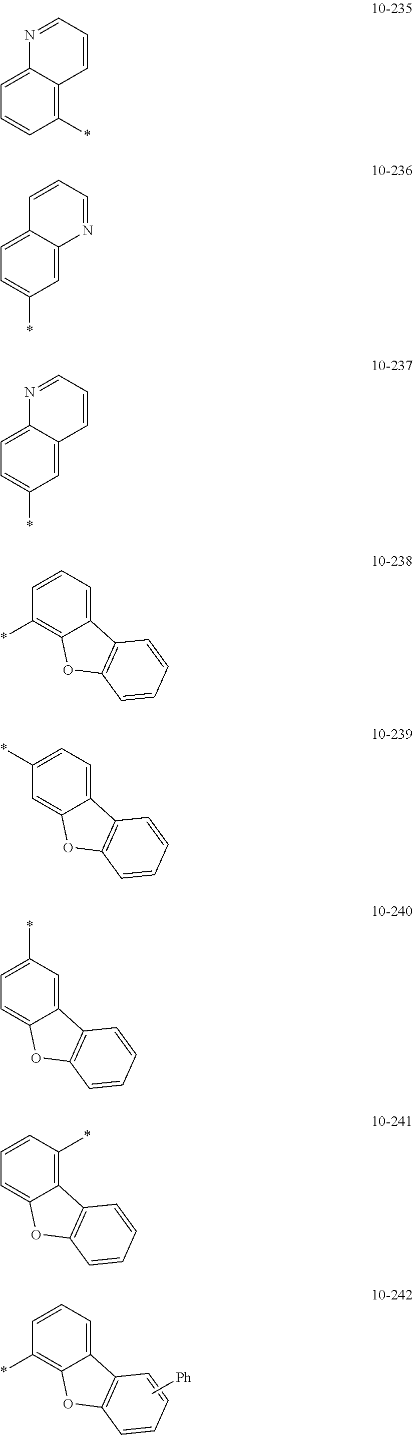

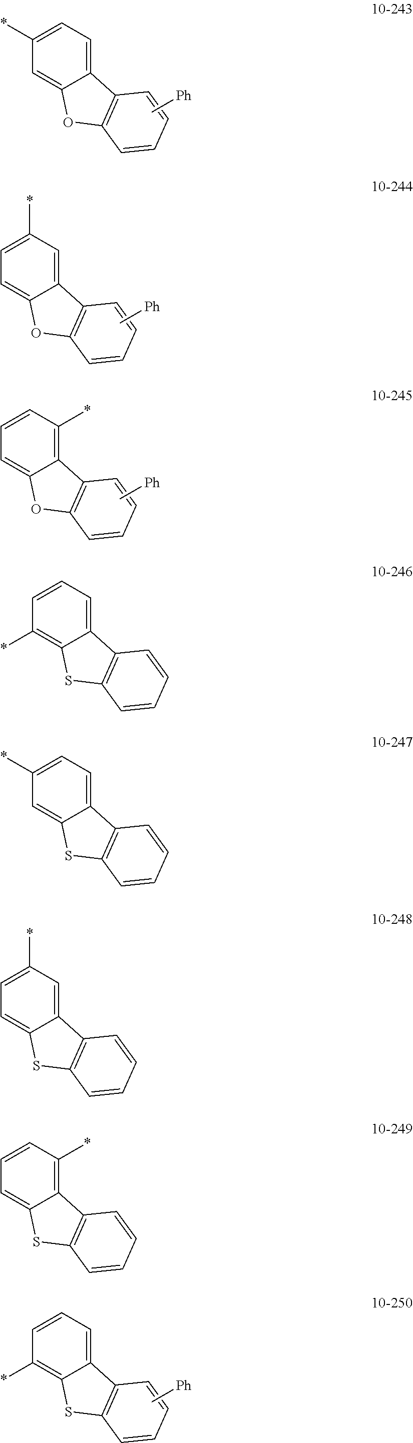

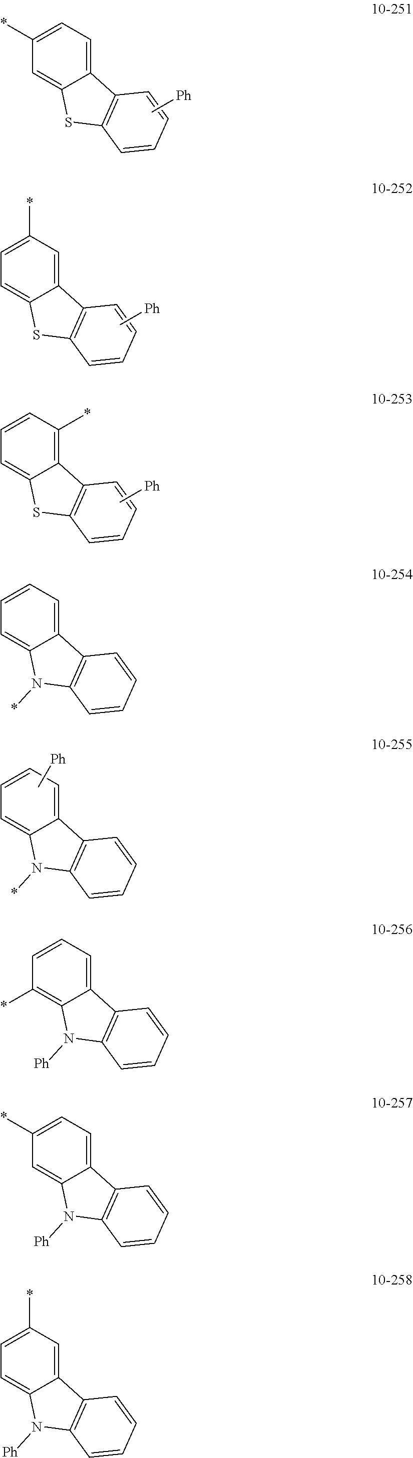

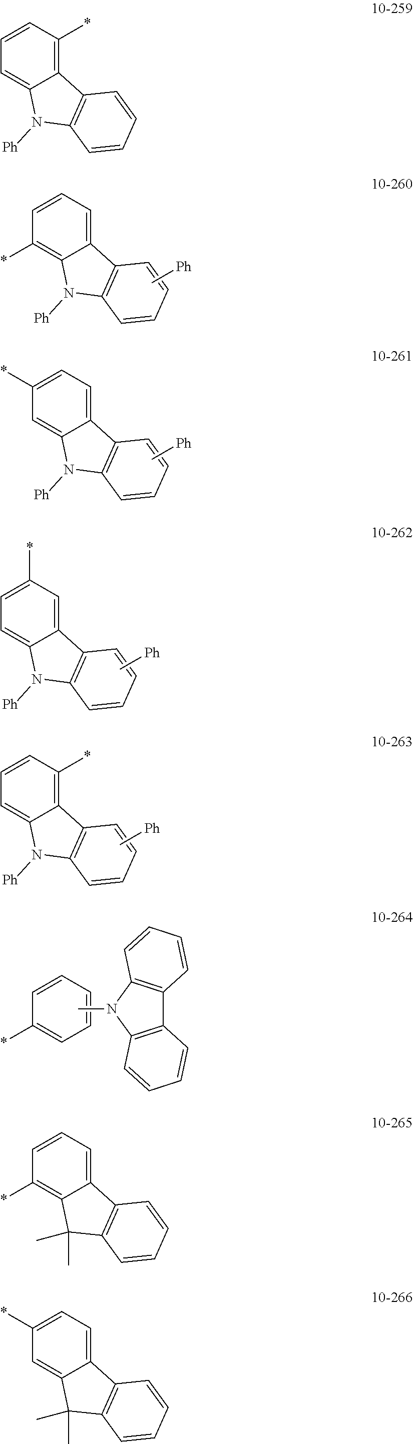

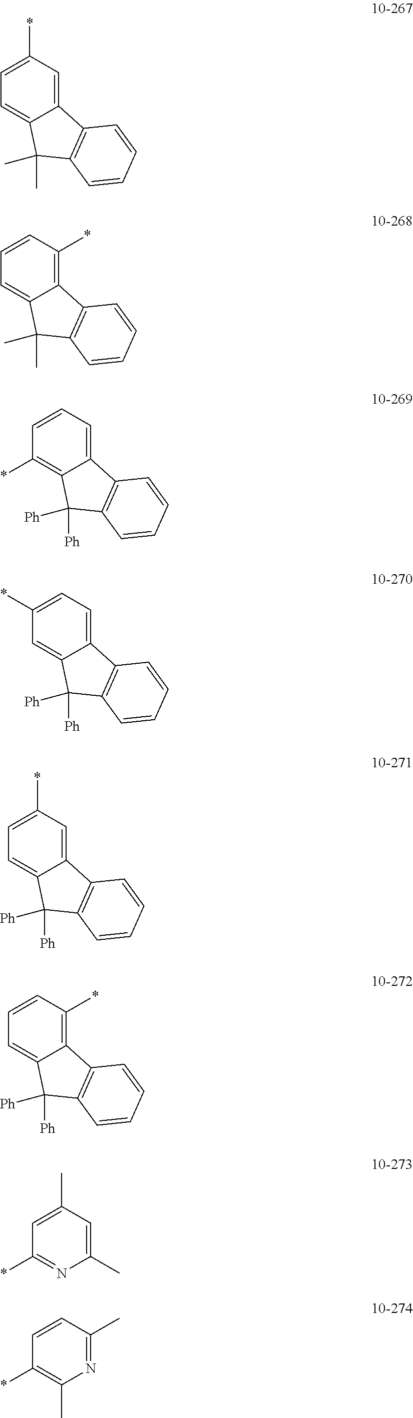

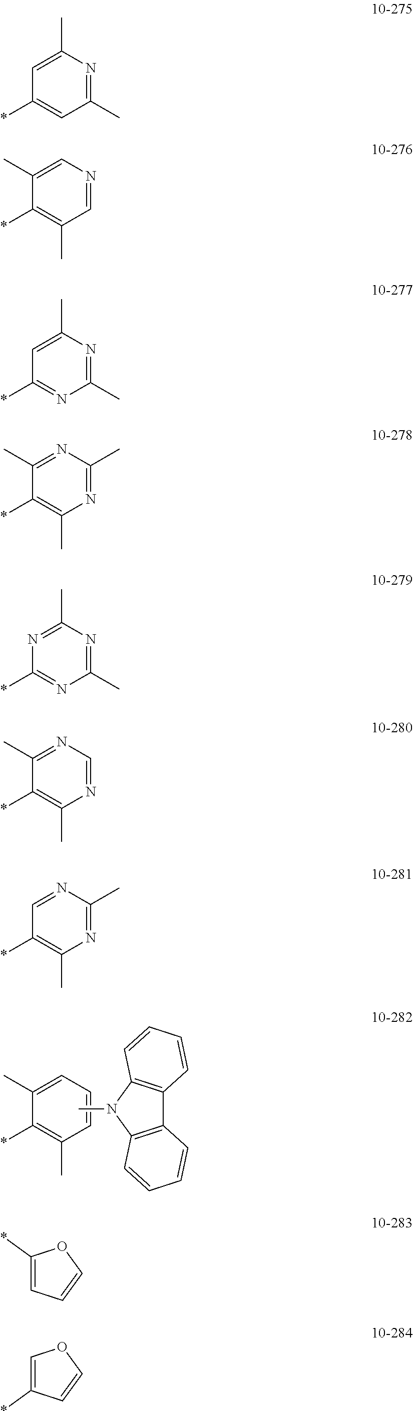

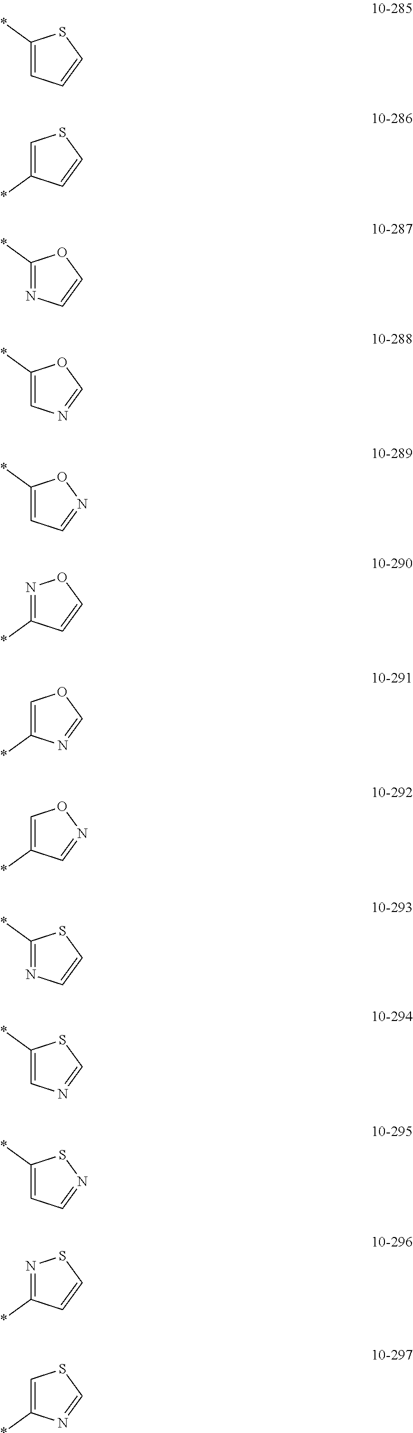

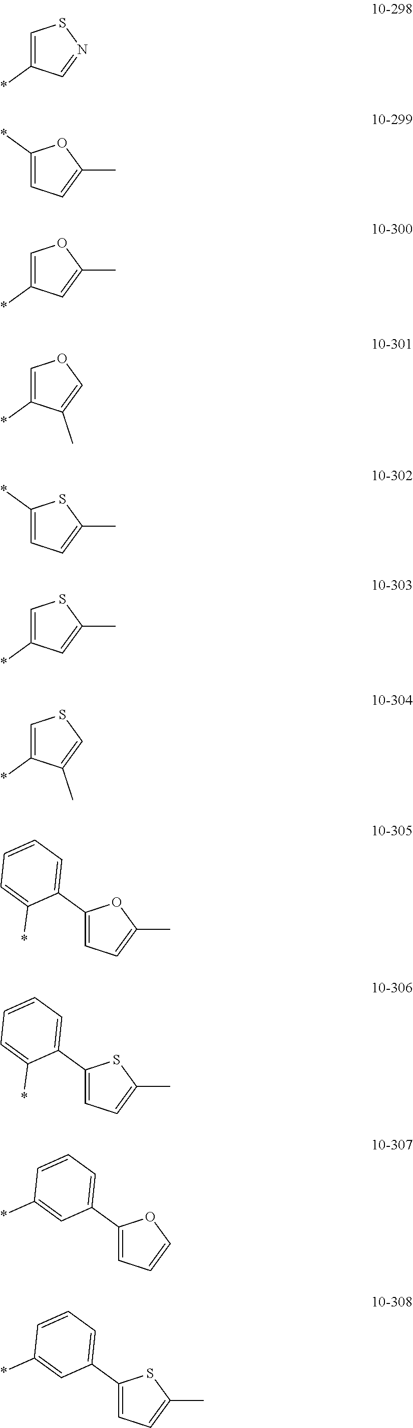

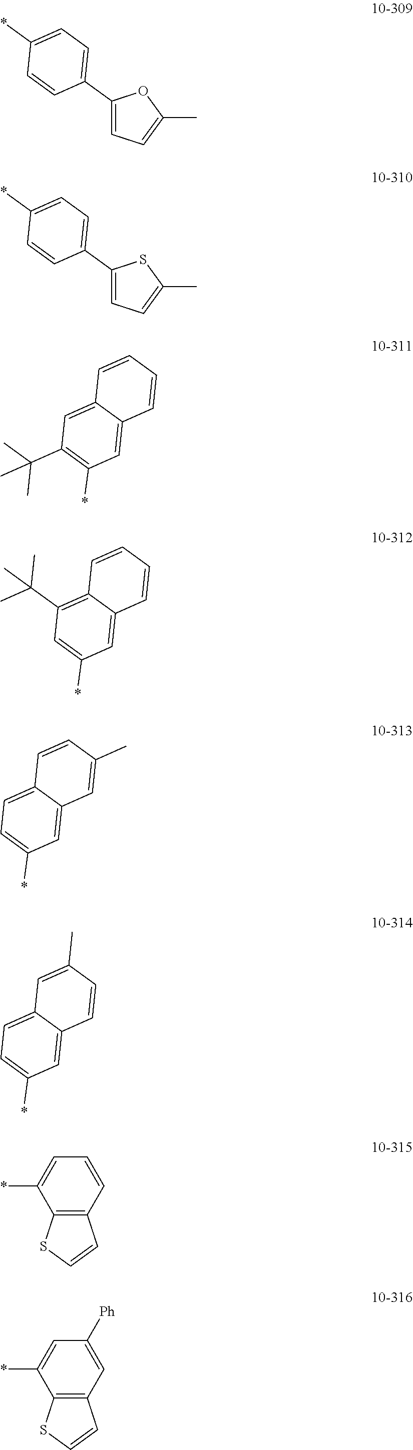

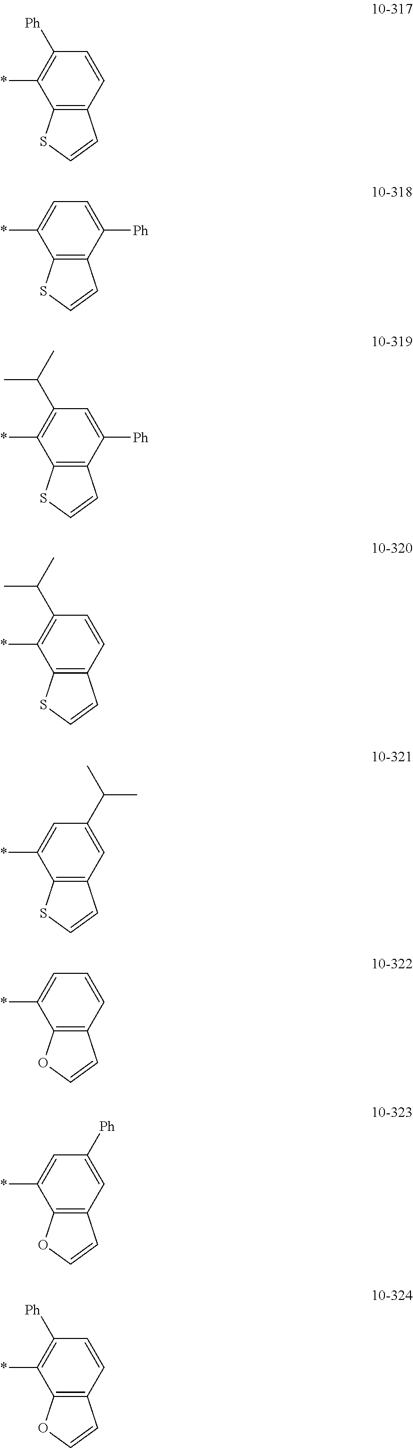

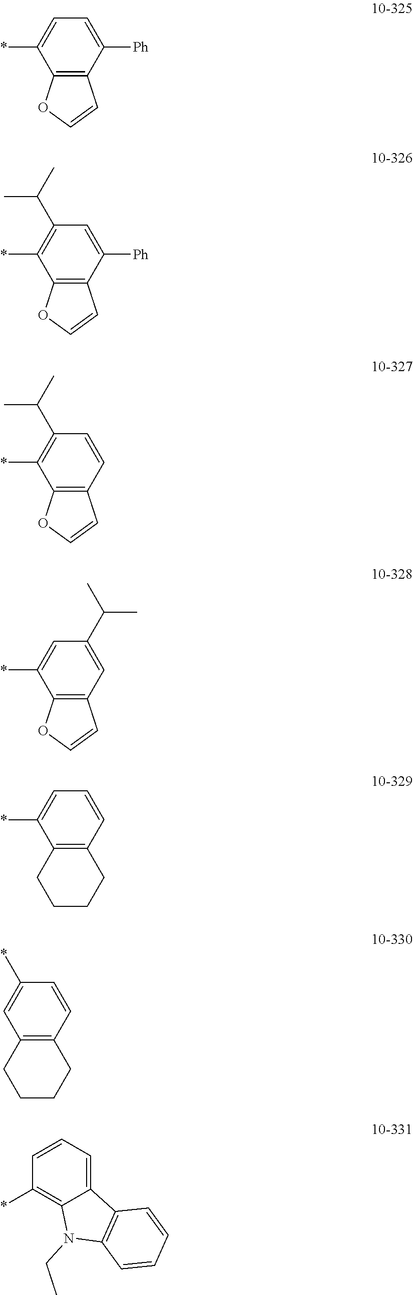

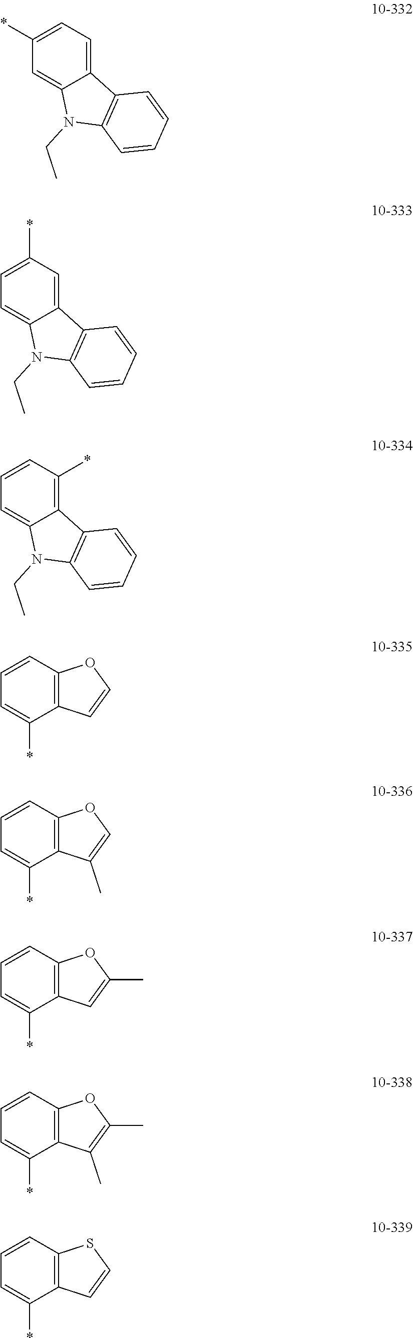

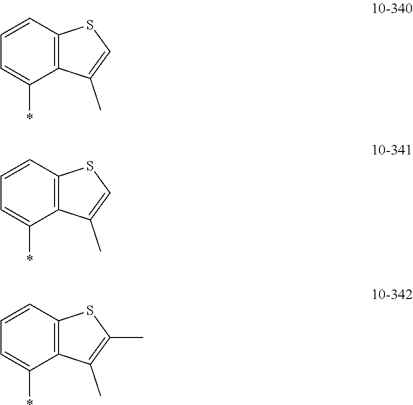

In Formulae 9-1 to 9-66, 10-1 to 10-118 and 10-201 to 10-342, * indicates a binding site to a neighboring atom, Ph indicates a phenyl group, and TMS indicates a trimethylsilyl group.

The “group represented by one of Formulae 9-1 to 9-66 in which a hydrogen is substituted with deuterium” may be, for example, a group represented by one of Formulae 9-501 to 9-514 or 9-601 to 9-638.

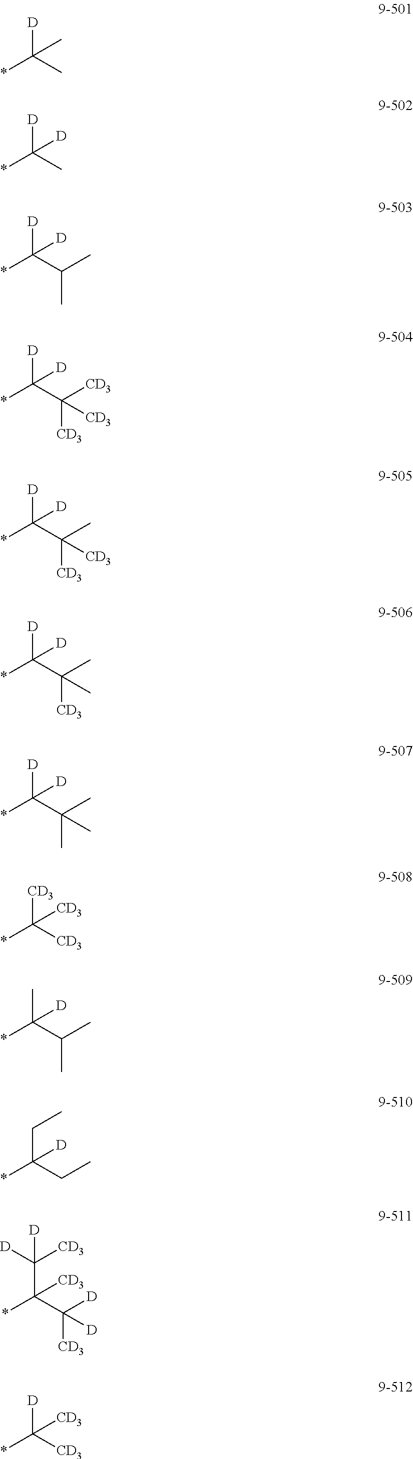

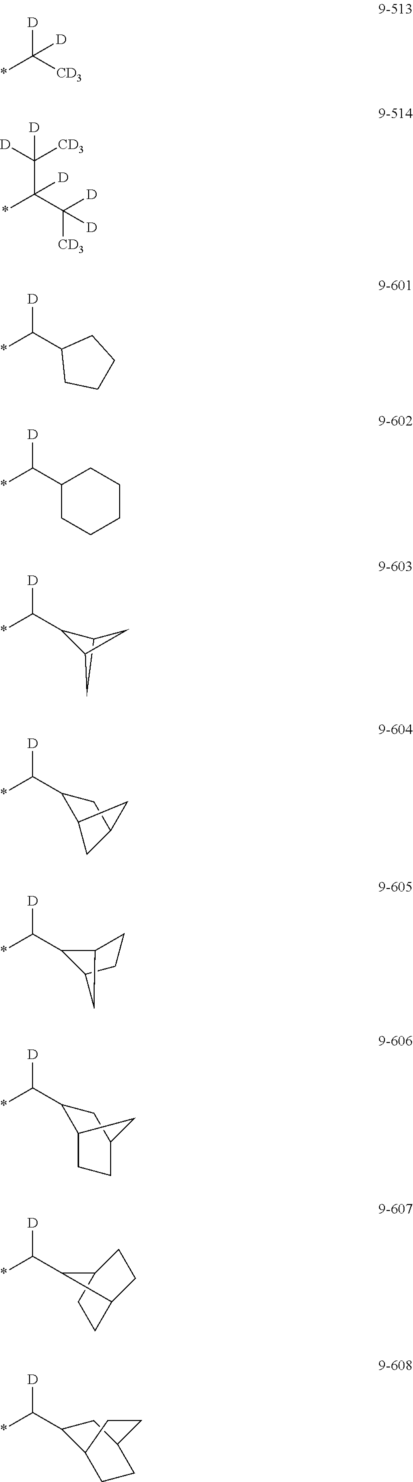

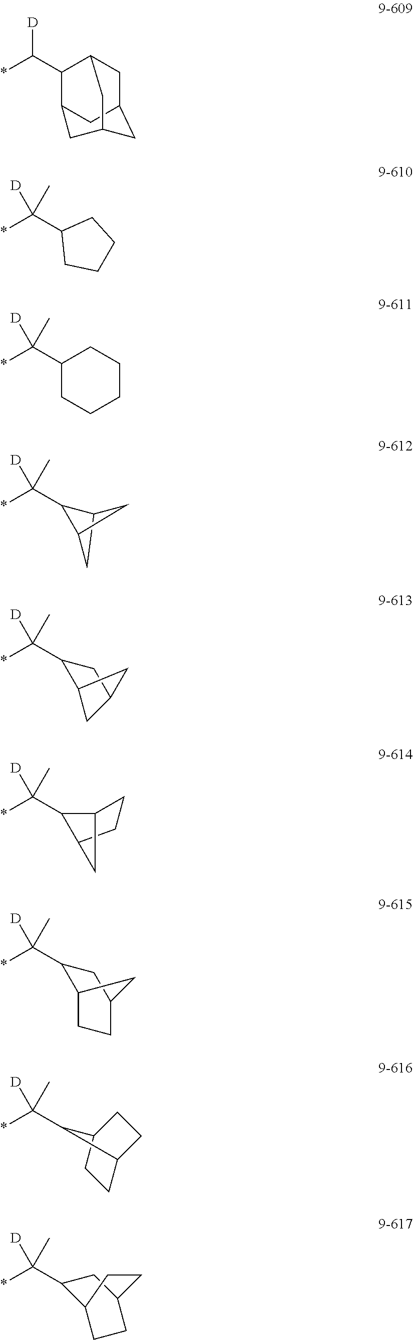

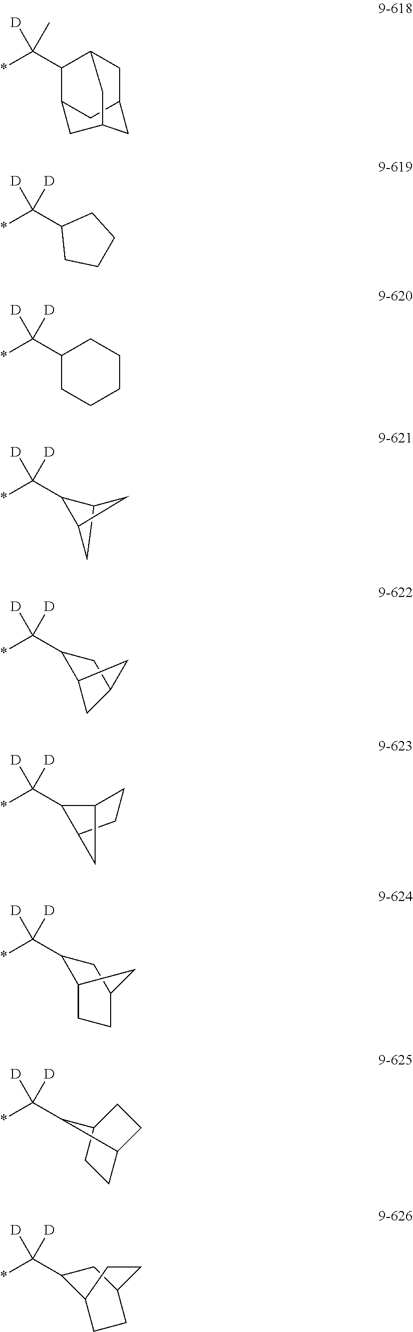

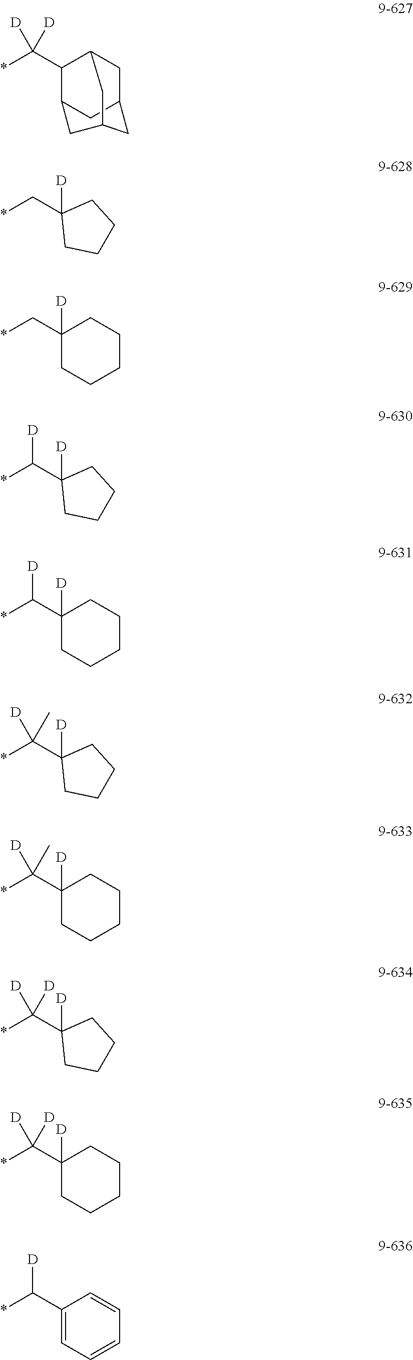

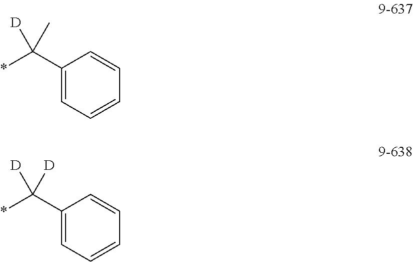

The “group represented by one of Formulae 10-1 to 10-118 in which a hydrogen is substituted with deuterium” may be, for example, a group represented by one of Formulae 10-501 to 10-552:

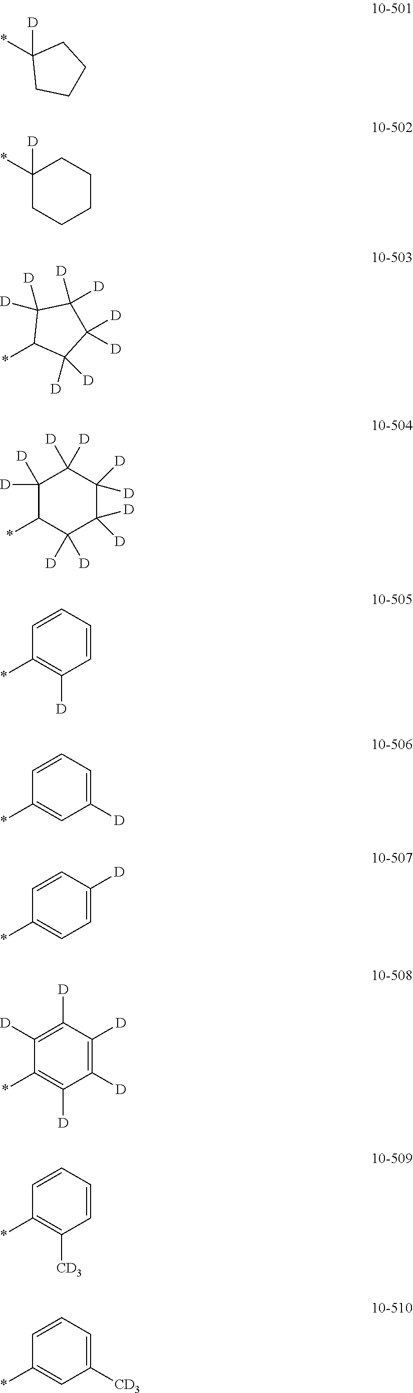

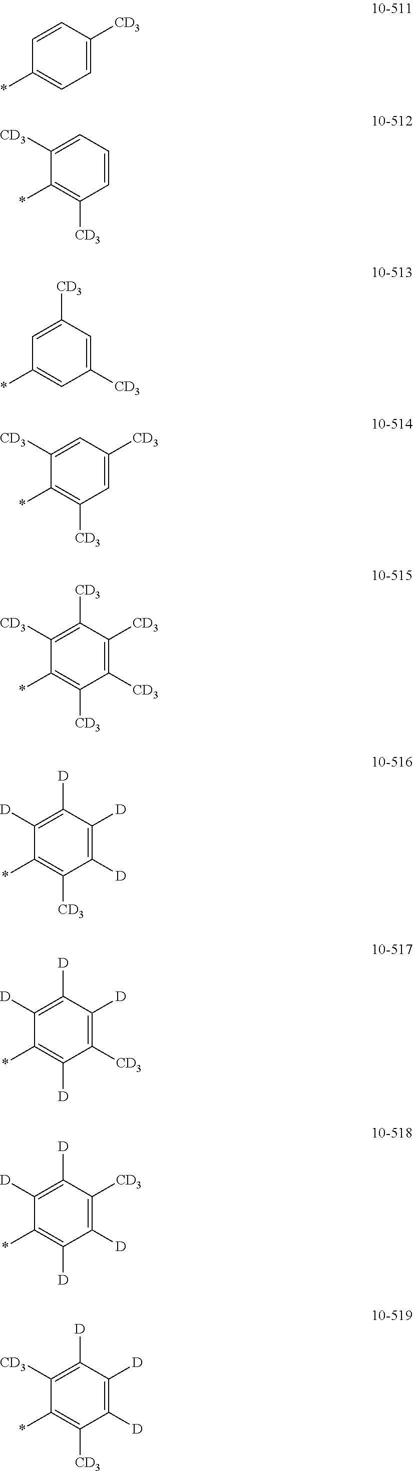

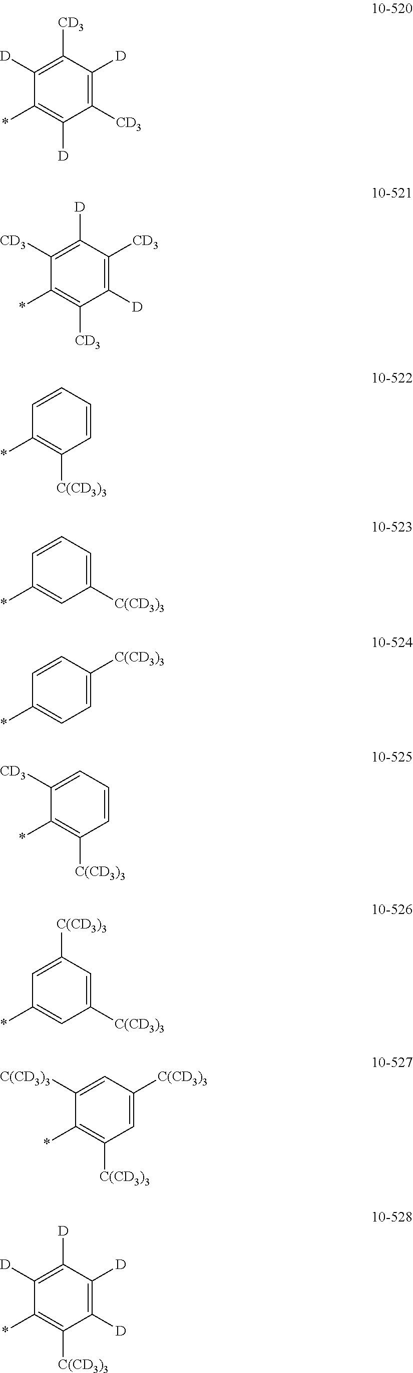

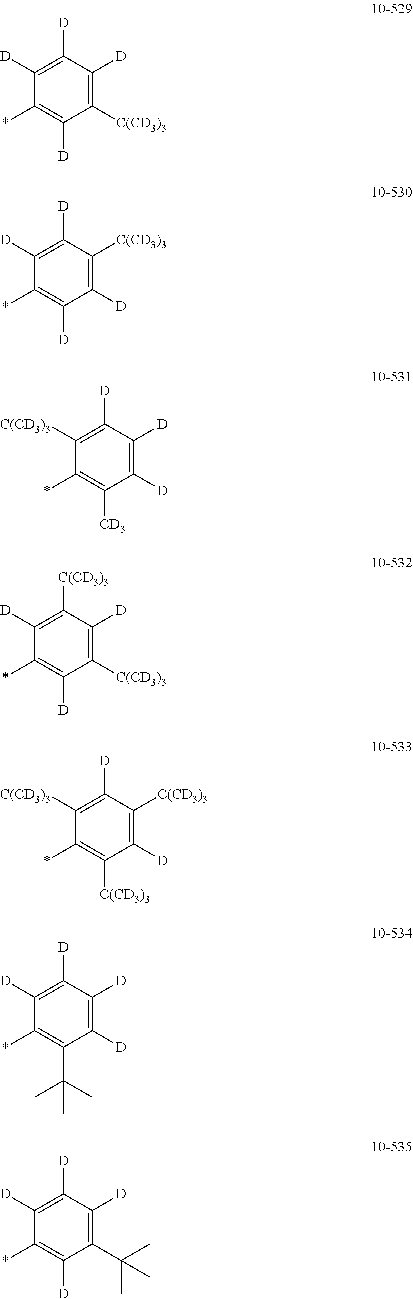

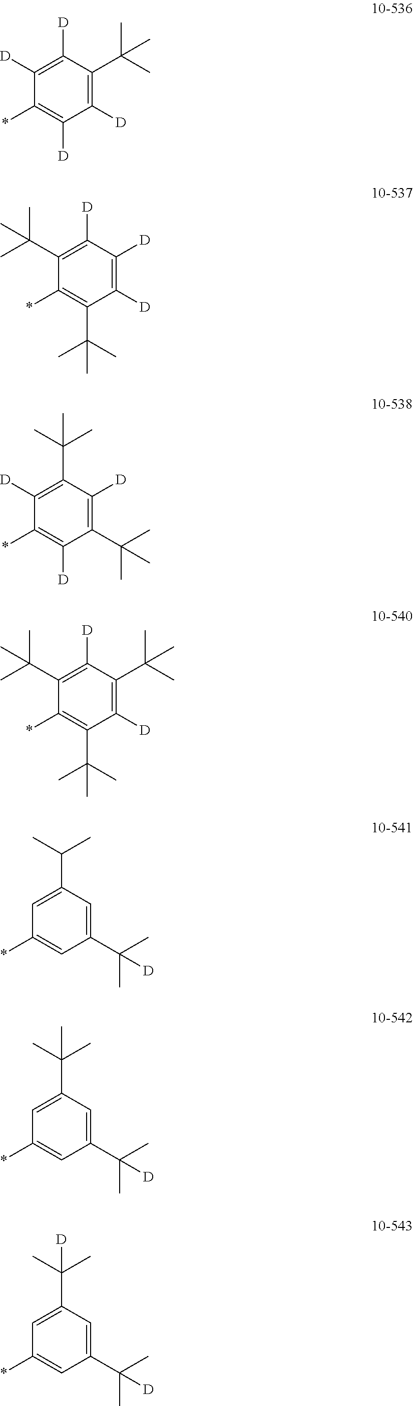

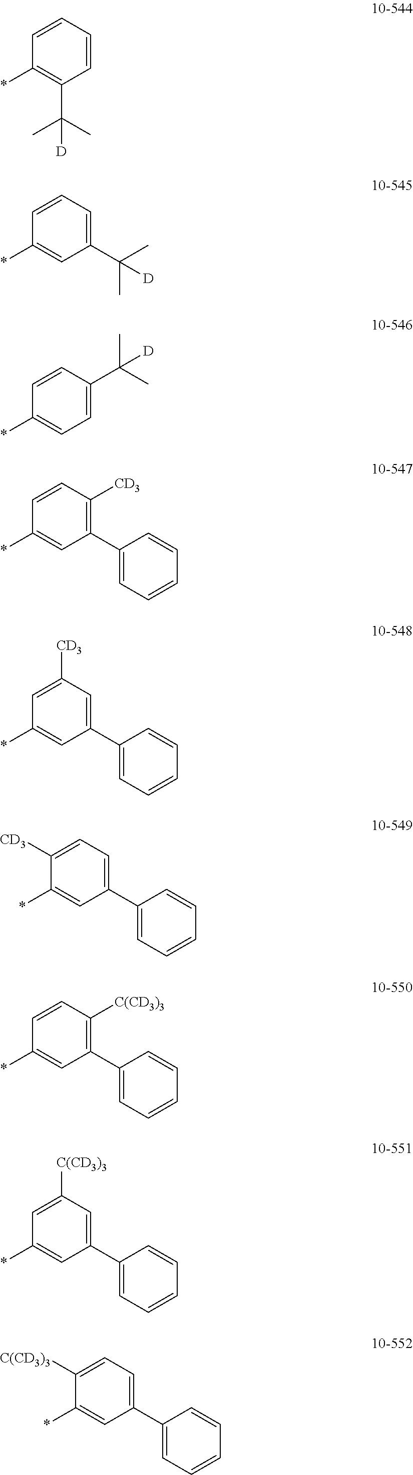

In an embodiment, in Formula 1, A₅₂ may be a group represented by one of Formulae 9-1 to 9-36 in which all hydrogens are substituted with deuterium, but embodiments of the present disclosure are not limited thereto.

In Formula 1, a1 to a4, a51, and a52 each indicate the number of *-[(L₁)_{b1}-(R₁)_{c1}], *-[(L₂)_{b2}-(R₂)_{c2}], *-[(L₃)_{b3}-(R₃)_{c3}], *-[(L₄)_{b4}-(R₄)_{c4}], A₅₁, and A₅₂, respectively, and may each independently be an integer from 0 to 10. When a1 is two or more, two or more *-[(L₁)_{b1}-(R₁)_{c1}] groups may be identical to or different from each other, when a2 is two or more, two or more *-[(L₂)_{b2}-(R₂)_{c2}] groups may be identical to or different from each other, when a3 is two or more, two or more *-[(L₃)_{b3}-(R₃)_{c3}] groups may be identical to or different from each other, when a4 is two or more, two or more *-[(L₄)_{b4}-(R₄)_{c4}] groups may be identical to or different from each other, when a51 is two or more, two or more A₅₁ groups may be identical to or different from each other, and when a52 is two or more, two or more A₅₂ groups may be identical to or different from each other, but embodiments of the present disclosure are not limited thereto.

For example, in Formula 1, a1 to a4, a51, and a52 may each independently be 0, 1, 2, 3, 4, 5, or 6, but embodiments of the present disclosure are not limited thereto.

For example, in Formula 1, a1 and a4 may each independently be 0, 1, 2, 3, or 4 and a2, a3, a51 and a52 may each independently be 0, 1, 2, or 3.

In Formula 1, the sum of a51 and a52 may be 1 or more. That is, in Formula 1, ring CY₅₁ may be substituted with a group represented by A₅₁, a group represented by A₅₂, or any combination thereof.

For example, the sum of a51 and a52 may be 1, 2, or 3. In an embodiment, the sum of a51 and a52 may be 1 or 2.

In Formula 1, a53 indicates the number of groups represented by

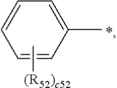

and may be an integer from 1 to 10. Since a53 in Formula 1 is not 0, ring CY₅₁ in

Formula 1 is substituted with a group represented by

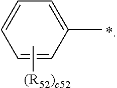

In an embodiment, in Formula 1,
L₁ to L₃ may be a single bond, and
R₁ to R₃ may each independently be:
hydrogen, deuterium, —F, a cyano group, C₁-C₂₀ alkyl group, or a C₁-C₂₀ alkoxy group;
a C₁-C₂₀ alkyl group or a C₁-C₂₀ alkoxy group, each substituted with deuterium, —F, —CD₃, —CD₂H, —CDH₂, —CF₃, —CF₂H, —CFH₂, a cyano group, a C₁-C₁₀ alkyl group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof; or
a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a phenyl group, a (C₁-C₂₀ alkyl)phenyl group, a biphenyl group, or a terphenyl group, each unsubstituted or substituted with deuterium, —F, —CD₃, —CD₂H, —CDH₂, —CF₃, —CF₂H, —CFH₂, a cyano group, a C₁-C₂₀ alkyl group, a deuterium-containing C₂-C₂₀ alkyl group (for example, *—C(CD₃)₃), a C₁-C₂₀ alkoxy group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof.

In an embodiment, in Formula 1, a3, and a4 may not be 0, and a group represented by *-(L₃)_{b3}-(R₃)_{c3}, and a group represented by *-(L₄)_{b4}-(R₄)_{c4} may not be hydrogen.

In an embodiment, in Formula 1, a1, a3, and a4 may not be 0, and a group represented by *-(L₁)_{b1}-(R₁)_{c1}, a group represented by *-(L₃)_{b3}-(R₃)_{c3}, and a group represented by *-(L₄)_{b4}-(R₄)_{c4} may not be hydrogen.

In an embodiment, in Formula 1, a1 may not be 0, a group represented by *-(L₁)_{b1}-(R₁)_{c1} may not be hydrogen, and a group represented by *-(L₁)_{b1}-(R₁)_{c1} in a number of a1 may include a deuterium.

In an embodiment, in Formula 1, a1 may be 2, two groups represented by *-(L₁)_{b1}-(R₁)_{c1} may not be hydrogen, and two groups represented by *-(L₁)_{b1}-(R₁)_{c1} may be identical to each other.

In an embodiment, in Formula 1, a1 may be 2, two groups represented by *-(L₁)_{b1}-(R₁)_{c1} may not be hydrogen, and two groups represented by *-(L₁)_{b1}-(R₁)_{c1} may be different from each other.

In an embodiment, in Formula 1, a4 may not be 0, a group represented by *-(L₄)_{b4}-(R₄)_{c4} may not be hydrogen, and a group represented by *-(L₄)_{b4}-(R₄)_{c4} in a number of a4 may include a deuterium.

In an embodiment, in Formula 1, a3 may not be 0, and a group represented by *-(L₃)_{b3}-(R₃)_{c3} in a number of a3 may satisfy Condition A and Condition B:
Condition A
L₃ is a single bond.
Condition B
R₃ is
hydrogen, deuterium, —F, a cyano group, C₁-C₂₀ alkyl group, or a C₁-C₂₀ alkoxy group;
a C₁-C₂₀ alkyl group or a C₁-C₂₀ alkoxy group, each substituted with deuterium, —F, —CD₃, —CD₂H, —CDH₂, —CF₃, —CF₂H, —CFH₂, a cyano group, a C₁-C₁₀ alkyl group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof; or
a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a phenyl group, a (C₁-C₂₀ alkyl)phenyl group, a biphenyl group, or a terphenyl group, each unsubstituted or substituted with deuterium, —F, —CD₃, —CD₂H, —CDH₂, —CF₃, —CF₂H, —CFH₂, a cyano group, a C₁-C₂₀ alkyl group, a deuterium-containing C₂-C₂₀ alkyl group, a C₁-C₂₀ alkoxy group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof.

In an embodiment, in Condition B, R₃ may not hydrogen.

In an embodiment, in Formula 1, a3 may not be 0, and a group represented by *-(L₃)_{b3}-(R₃)_{c3} in a number of a3 may satisfy Condition A and Condition B(1):
Condition A
L₃ is a single bond.
Condition B(1)
R₃ is
a C₄-C₂₀ alkyl group unsubstituted or substituted with deuterium, —F, —CD₃, —CD₂H, —CDH₂, —CF₃, —CF₂H, —CFH₂, a cyano group, a C₁-C₁₀ alkyl group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof; or
a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a phenyl group, a (C₁-C₂₀ alkyl)phenyl group, a biphenyl group, or a terphenyl group, each unsubstituted or substituted with deuterium, —F, —CD₃, —CD₂H, —CDH₂, —CF₃, —CF₂H, —CFH₂, a cyano group, a C₁-C₂₀ alkyl group, a deuterium-containing C₂-C₂₀ alkyl group, a C₁-C₂₀ alkoxy group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof.

In an embodiment, in Formula 1, a4 may not be 0, and a group represented by *-(L₄)_{b4}-(R₄)_{c4} in a number of a4 may satisfy Condition 1, Condition 2, or combination thereof:
Condition 1
R₄ in a number of c4 is a substituted or unsubstituted C₆-C₆₀ aryl group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group.
Condition 2
L₄ is not a single bond.

In an embodiment, in Formula 1, a4 may not be 0, and a group represented by *-(L₄)_{b4}-(R₄)_{c4} in a number of a4 may satisfy Condition 1(1), Condition 2(1), or combination thereof:
Condition 1(1)
R₄ in a number of c4 is a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a phenyl group, a (C₁-C₂₀ alkyl)phenyl group, a biphenyl group, or a terphenyl group, each unsubstituted or substituted with deuterium, —F, —CD₃, —CD₂H, —CDH₂, —CF₃, —CF₂H, —CFH₂, a cyano group, a C₁-C₂₀ alkyl group, a deuterium-containing C₂-C₂₀ alkyl group, a C₁-C₂₀ alkoxy group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof.
Condition 2(1)
L₄ is a benzene group unsubstituted or substituted with deuterium, —F, —CD₃, —CD₂H, —CDH₂, —CF₃, —CF₂H, —CFH₂, a cyano group, a C₁-C₂₀ alkyl group, a deuterium-containing C₂-C₂₀ alkyl group, a C₁-C₂₀ alkoxy group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof.

In an embodiment, in Formula 1, a4 may not be 0, and a group represented by *-(L₄)_{b4}-(R₄)_{c4} in a number of a4 may satisfy Condition 3, Condition 4, Condition 5, or combination thereof:
Condition 3
R₄ in a number of c4 is a substituted C₆-C₆₀ aryl group.
Condition 4
L₄ is a C₅-C₃₀ carbocyclic group substituted with an R₁₀ₐ group,
Condition 5
L₄ is a C₅-C₃₀ carbocyclic group that is unsubstituted or substituted with an R₁₀ₐ group and R₄ is not hydrogen.

In an embodiment, in Formula 1, a4 may not be 0, and a group represented by *-(L₄)_{b4}-(R₄)_{c4} in a number of a4 may satisfy Condition 3(1), Condition 4(1), Condition 5(1), or combination thereof:
Condition 3(1)
R₄ in a number of c4 is a phenyl group, a (C₁-C₂₀ alkyl)phenyl group, a biphenyl group, or a terphenyl group, each substituted with deuterium, —F, —CD₃, —CD₂H, —CDH₂, —CF₃, —CF₂H, —CFH₂, a cyano group, a C₁-C₂₀ alkyl group, a deuterium-containing C₂-C₂₀ alkyl group, a C₁-C₂₀ alkoxy group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof.
Condition 4(1)
L₄ is a benzene group substituted with deuterium, —F, —CD₃, —CD₂H, —CDH₂, —CF₃, —CF₂H, —CFH₂, a cyano group, a C₁-C₂₀ alkyl group, a deuterium-containing C₂-C₂₀ alkyl group, a C₁-C₂₀ alkoxy group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof.
Condition 5(1)
L₄ is a benzene group unsubstituted or substituted with deuterium, —F, —CD₃, —CD₂H, —CDH₂, —CF₃, —CF₂H, —CFH₂, a cyano group, a C₁-C₂₀ alkyl group, a deuterium-containing C₂-C₂₀ alkyl group, a C₁-C₂₀ alkoxy group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof, and
R₄ is
deuterium, —F, a cyano group, C₁-C₂₀ alkyl group, or a C₁-C₂₀ alkoxy group;
a C₁-C₂₀ alkyl group or a C₁-C₂₀ alkoxy group, each substituted with deuterium, —F, —CD₃, —CD₂H, —CDH₂, —CF₃, —CF₂H, —CFH₂, a cyano group, a C₁-C₁₀ alkyl group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof; or
a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a phenyl group, a (C₁-C₂₀ alkyl)phenyl group, a biphenyl group, or a terphenyl group, each unsubstituted or substituted with deuterium, —F, —CD₃, —CD₂H, —CDH₂, —CF₃, —CF₂H, —CFH₂, a cyano group, a C₁-C₂₀ alkyl group, a deuterium-containing C₂-C₂₀ alkyl group, a C₁-C₂₀ alkoxy group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof.

In an embodiment, in Formula 1, a51 and a52 may each independently be 0, 1 or 2, the sum of a51 and a52 may be 1 or 2, and a53 may be 1 or 2.

* and *′ each indicate a binding site to a neighboring atom.

For example, a group represented by

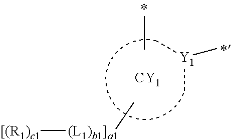

in Formula 1 may be a group represented by one of Formulae A1(1) to A1(30):

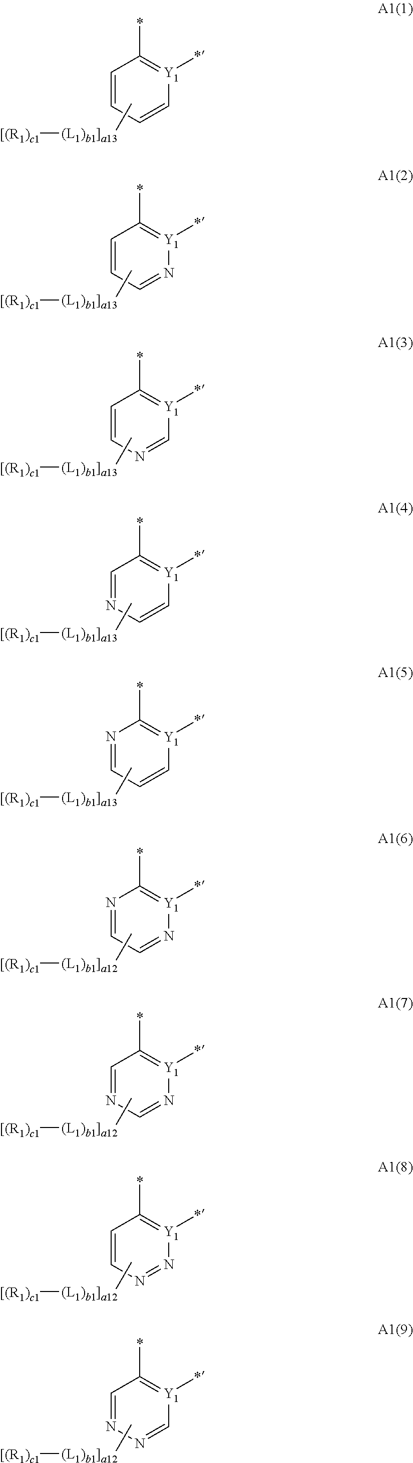

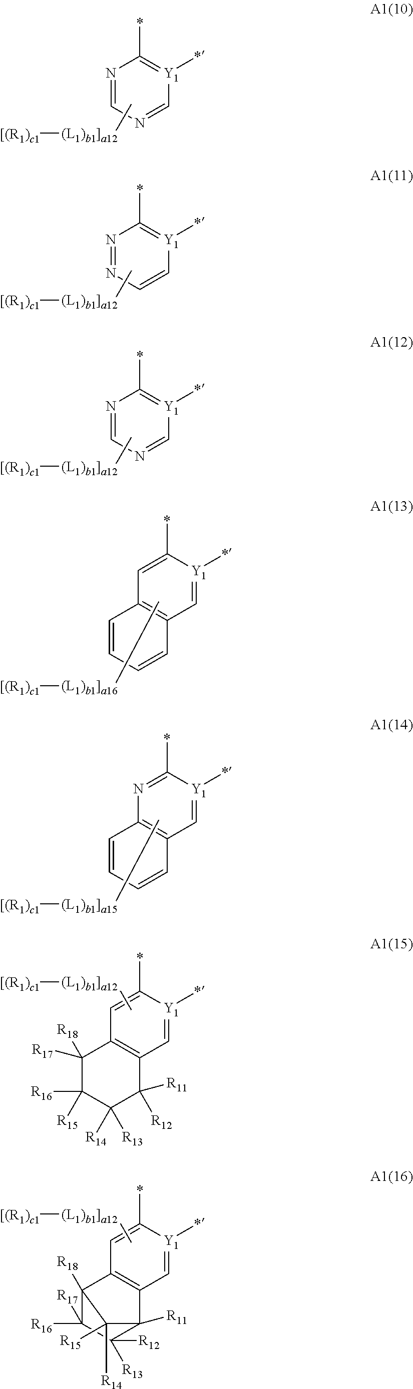

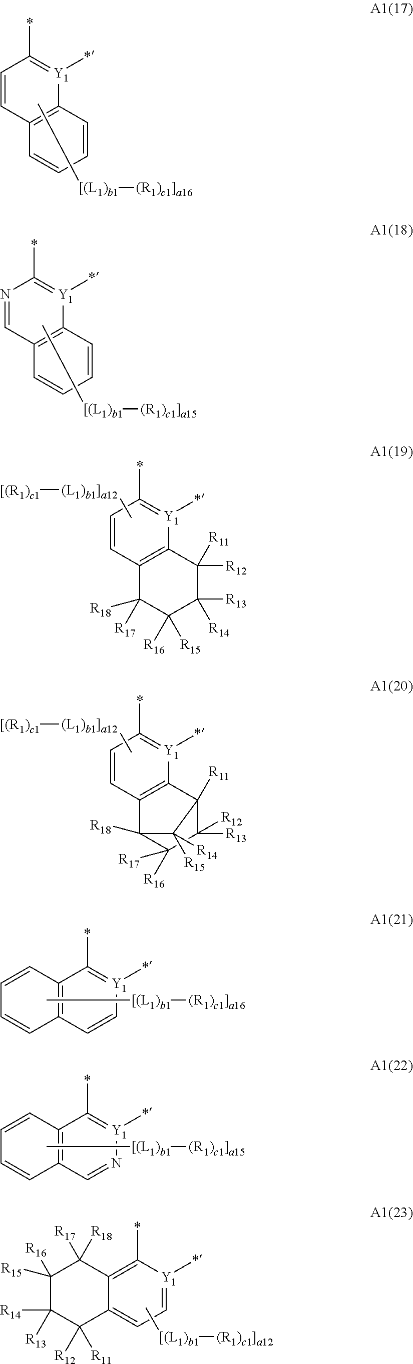

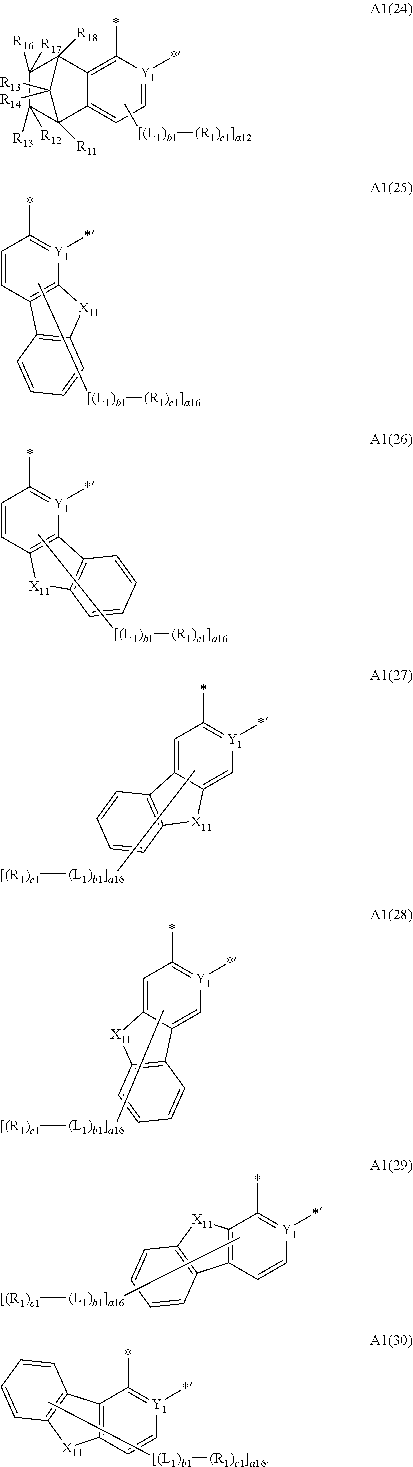

In Formulae A1(1) to A1(30),
Y₁, L₁, b1, R₁, and c1 may each independently be the same as described herein,
X₁₁ may be O, S, N(R₁₁), C(R₁₁)(R₁₂), or Si(R₁₁)(R₁₂),
R₁₁ to R₁₈ may each independently be the same as described in connection with R₁,
a12 may be an integer from 0 to 2,
a13 may be an integer from 0 to 3,
a14 may be an integer from 0 to 4,
a15 may be an integer from 0 to 5,
a16 may be an integer from 0 to 6,
*′ indicates a binding site to X₁ in Formula 1, and
indicates a binding site to Y₃ in Formula 1.

The group represented by

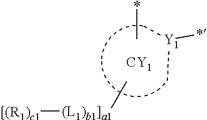

in Formula 1 may include deuterium, a deuterium-containing C₁-C₆₀ alkyl group unsubstituted or substituted with a C₃-C₁₀ cycloalkyl group, or any combination thereof. The number of deuterium atoms in the group represented by

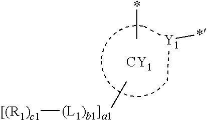

in Formula 1 may be from 1 to 20, for example, from 1 to 15, from 1 to 10, or from 1 to 5.

In an embodiment, a group represented by

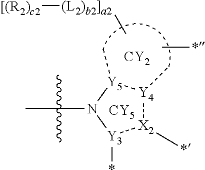

in Formula 1 may be a group represented by one of Formulae A2(1) to A2(4):

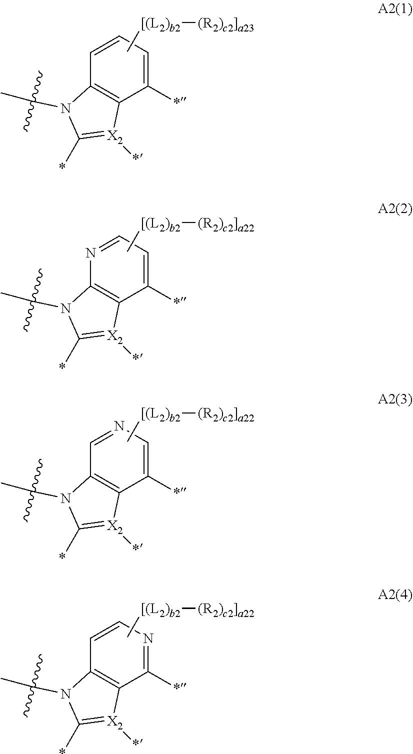

In Formulae A2(1) to A2(4),
X₂, L₂, b2, R₂, and c2 may each independently be the same as described herein,
a22 may be an integer from 0 to 2,
a23 may be an integer from 0 to 3,
[symbol] indicates a binding site to L₅₁ in Formula 1,
*″ indicates a binding site to ring CY₃ in Formula 1,
*′ indicates a binding site to M in Formula 1, and
* indicates a binding site to ring CY₁ in Formula 1.

The group represented by

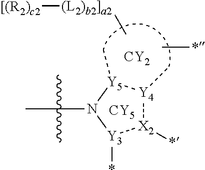

in Formula 1 may include deuterium, a deuterium-containing C₁-C₆₀ alkyl group unsubstituted or substituted with a C₃-C₁₀ cycloalkyl group, or any combination thereof. The number of deuterium atoms in the group represented by

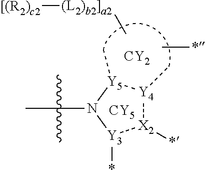

in Formula 1 may be from 1 to 20, for example, from 1 to 15, from 1 to 10, or from 1 to 5.

In an embodiment, a group represented by

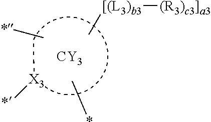

in Formula 1 may be a group represented by one of Formulae A3(1) to A3(17):

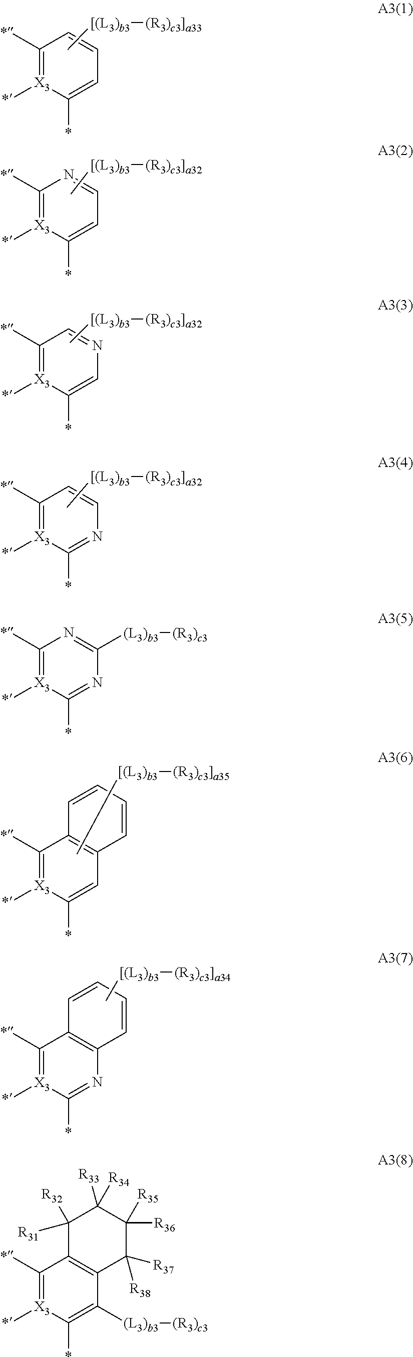

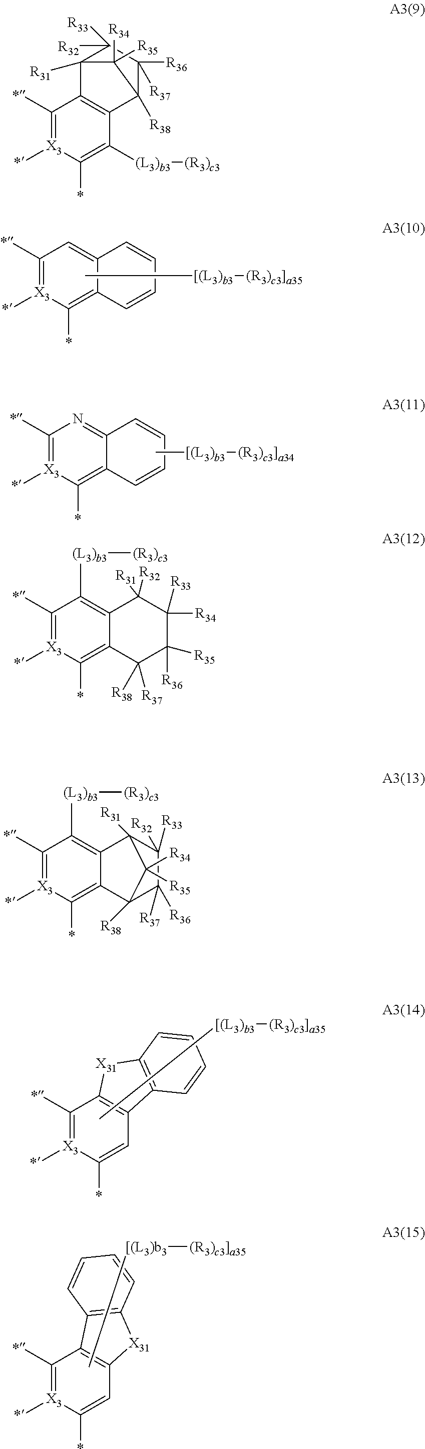

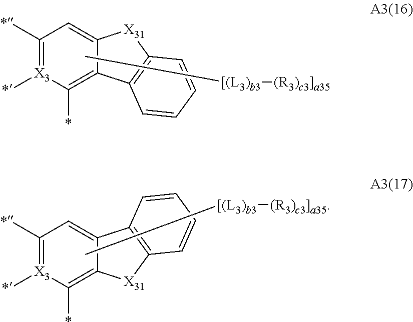

In Formulae A3(1) to A3(17),
X₃, L₃, b3, R₃, and c3 may each independently be the same as described herein,
X₃₁ may be O, S, N(R₃₁), C(R₃₁)(R₃₂), or Si(R₃₁)(R₃₂),
R₃₁ to R₃₈ may each independently be the same as described in connection with
a32 may be an integer from 0 to 2,
a33 may be an integer from 0 to 3,
a34 may be an integer from 0 to 4,
a35 may be an integer from 0 to 5,
*″ indicates a binding site to ring CY₂ in Formula 1,
*′ indicates a binding site to M in Formula 1, and
indicates a binding site to T₁ in Formula 1.

The group represented by

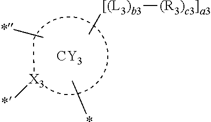

in Formula 1 may include deuterium, a deuterium-containing C₁-C₆₀ alkyl group unsubstituted or substituted with a C₃-C₁₀ cycloalkyl group, or any combination thereof. The number of deuterium atoms in the group represented by

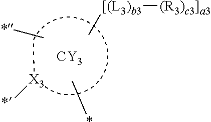

in Formula 1 may be from 1 to 20, for example, from 1 to 15, from 1 to 10, or from 1 to 5.

In an embodiment, a group represented by

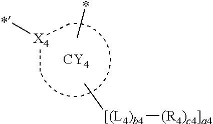

in Formula 1 may be a group represented by one of Formulae A4(1) to A4(45):

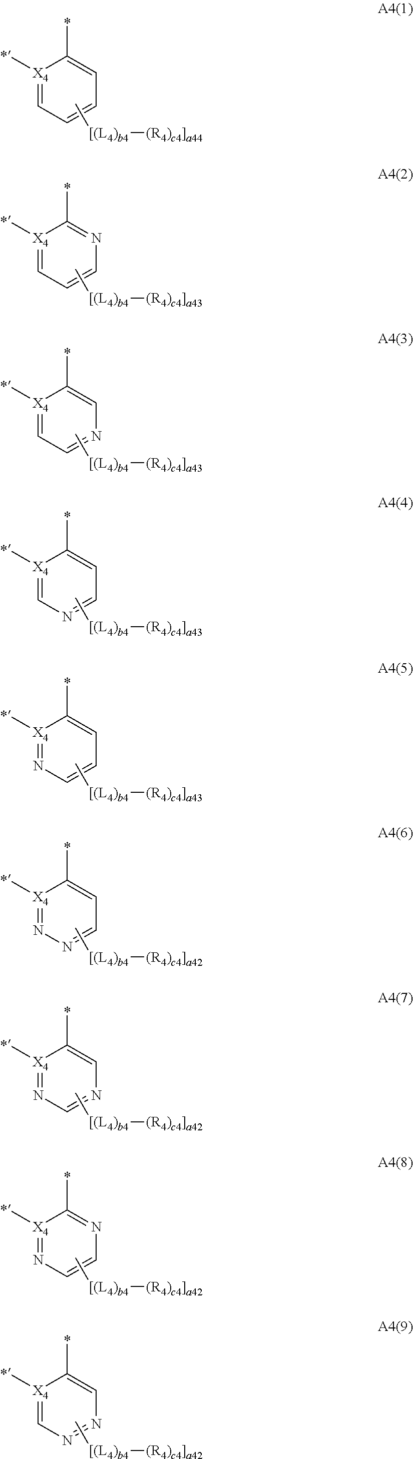

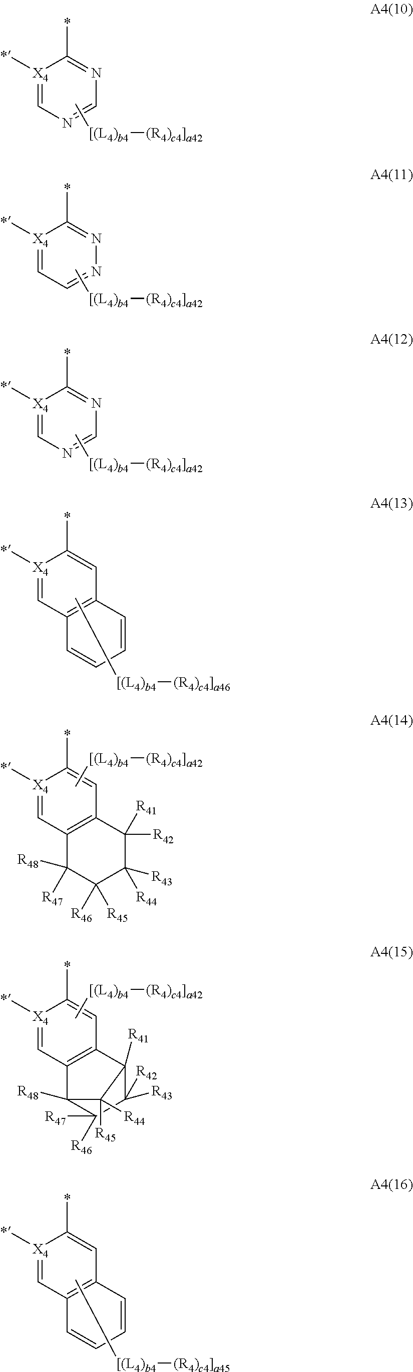

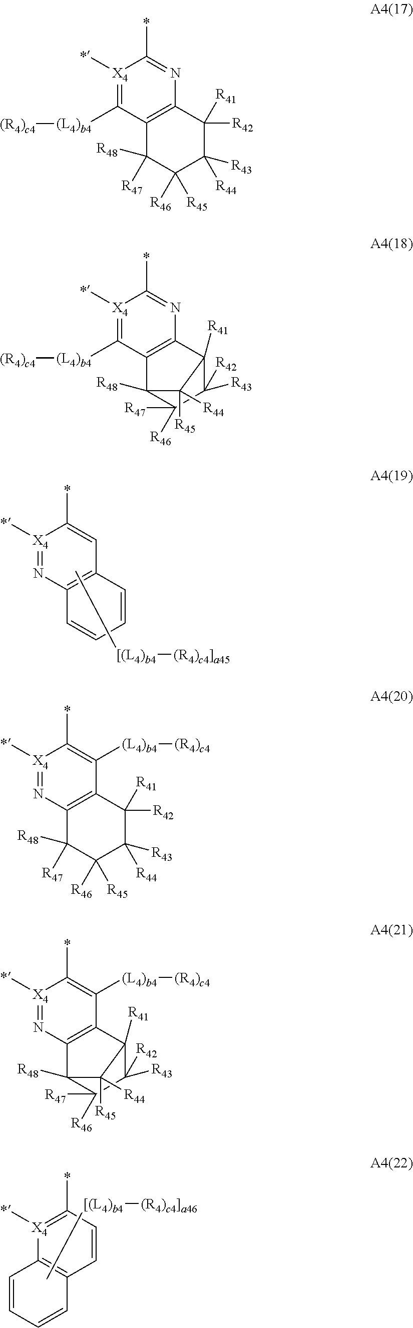

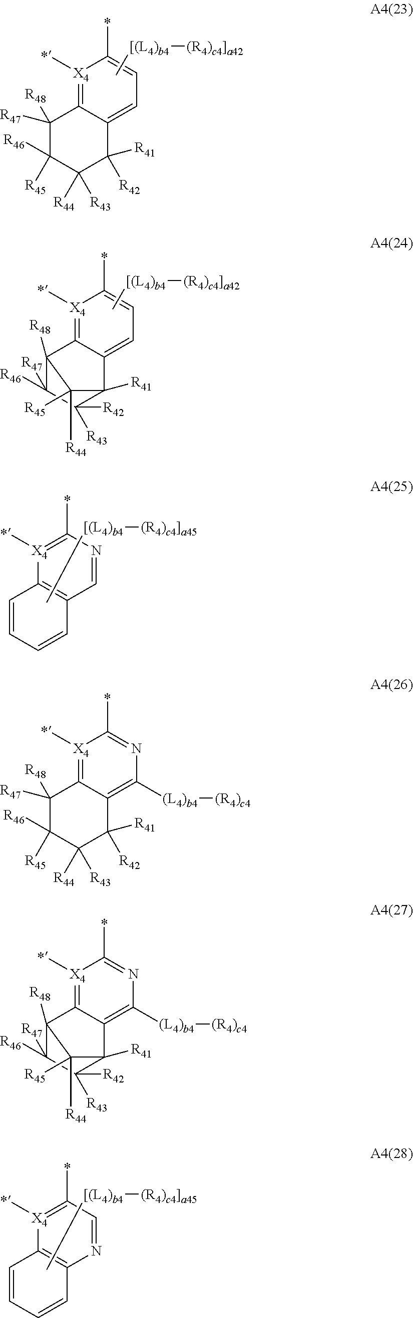

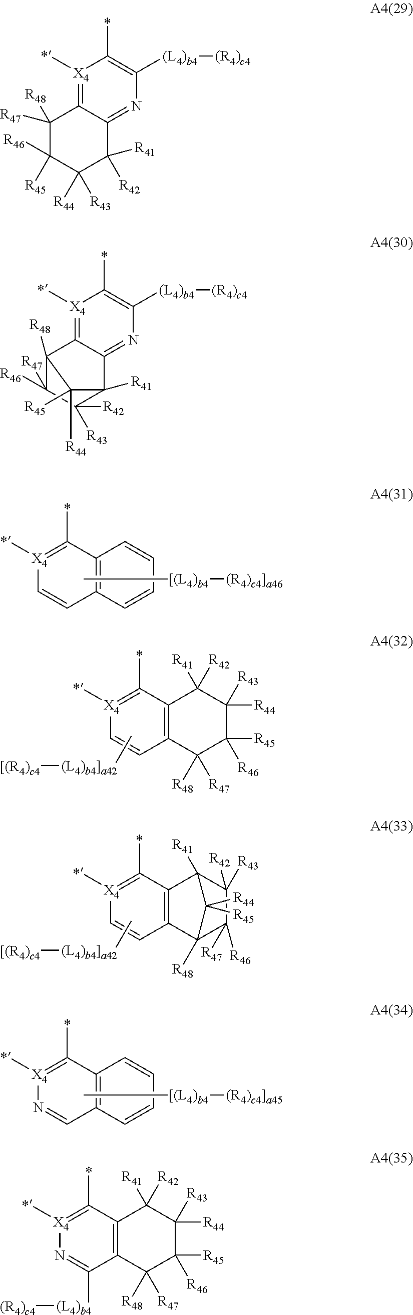

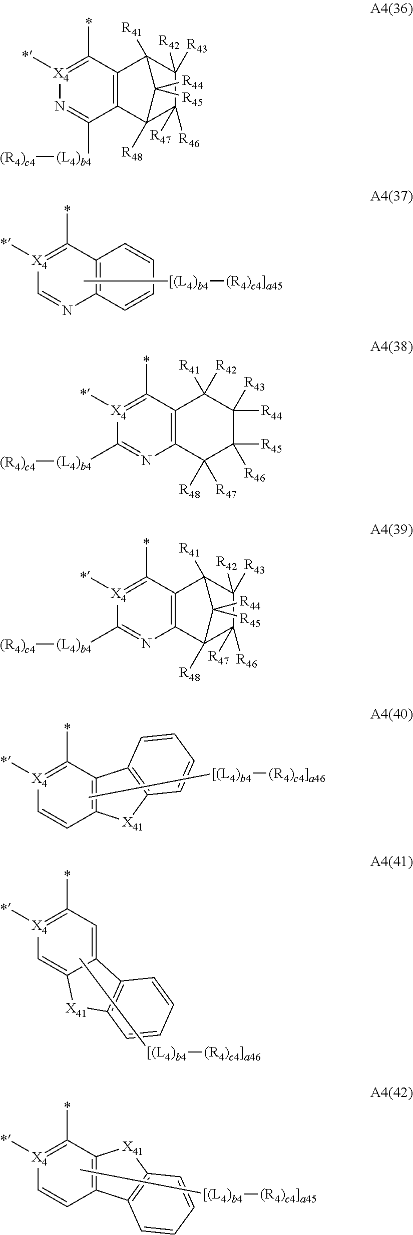

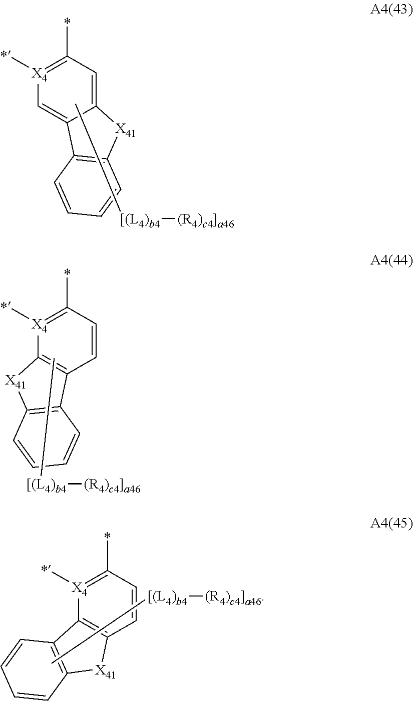

In Formulae A4(1) to A4(45),
X₄, L₄, b4, R₄, and c4 may each independently be the same as described herein,
X₄₁ may be O, S, N(R₄₁), C(R₄₁)(R₄₂), or Si(R₄₁)(R₄₂),
R₄₁ to R₄₈ may each independently be the same as described in connection with R₄,
a42 may be an integer from 0 to 2,
a43 may be an integer from 0 to 3,
a44 may be an integer from 0 to 4,
a45 may be an integer from 0 to 5,
a46 may be an integer from 0 to 6,
*′ indicates a binding site to M in Formula 1, and
indicates a binding site to T₁ in Formula 1.

The group represented by

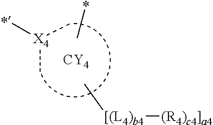

in Formula 1 may include deuterium, a deuterium-containing C₁-C₆₀ alkyl group unsubstituted or substituted with a C₃-C₁₀ cycloalkyl group, or any combination thereof. The number of deuterium atoms in the group represented by

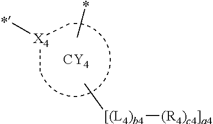

in Formula 1 may be from 1 to 20, for example, from 1 to 15, from 1 to 10, or from 1 to 5.

In an embodiment, in Formula 1, a group represented by

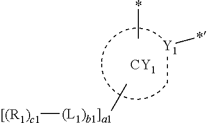

may be a group represented by one of Formulae CY1(1) to CY1(8), and/or
a group represented by

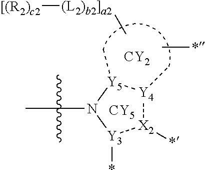

may be a group represented by one of Formulae CY2(1) to CY2(4), and/or
a group represented by

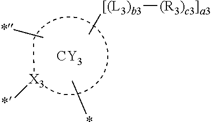

may be a group represented by one of Formulae CY3(1) to CY3(24), and/or
a group represented by

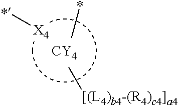

may be a group represented by one of Formulae CY4(1) to CY4(74), but embodiments of the present disclosure are not limited thereto:

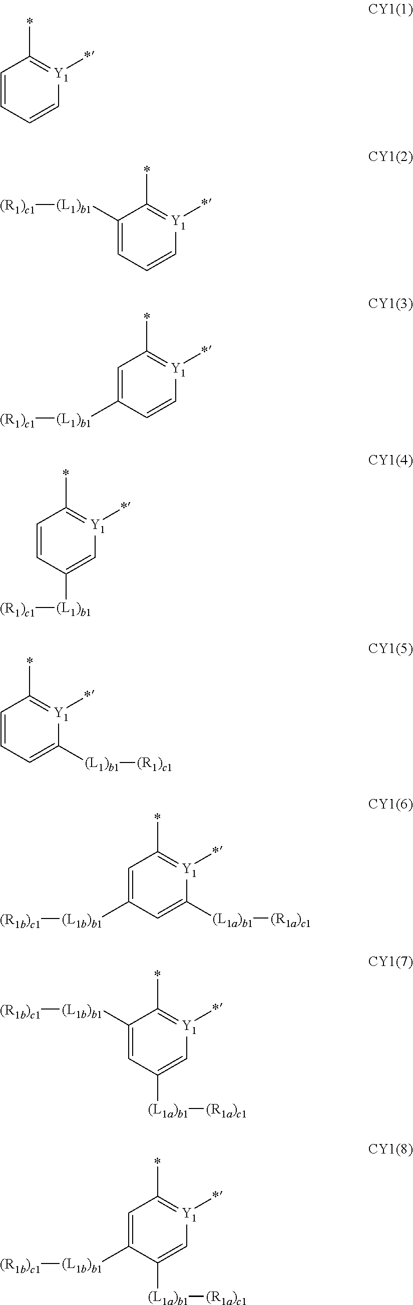

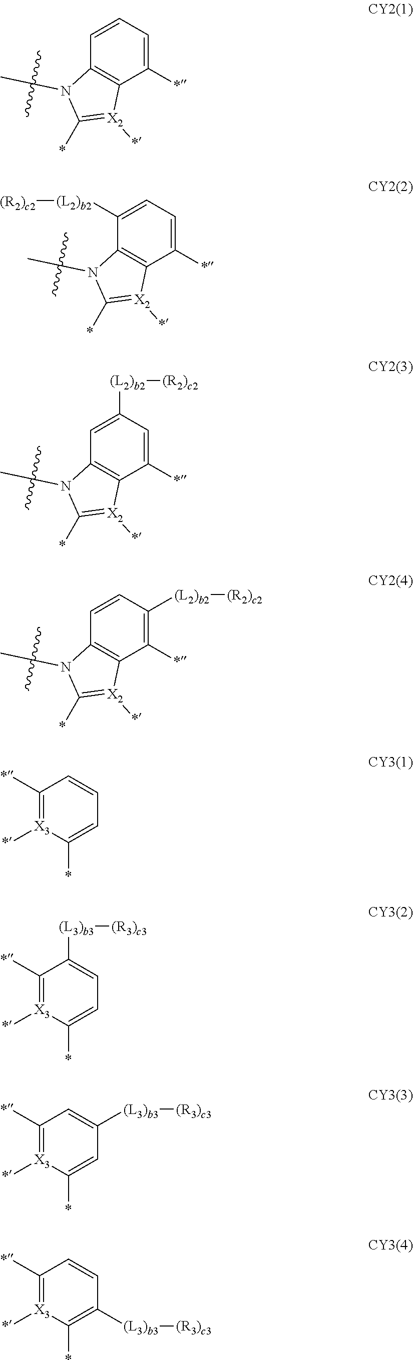

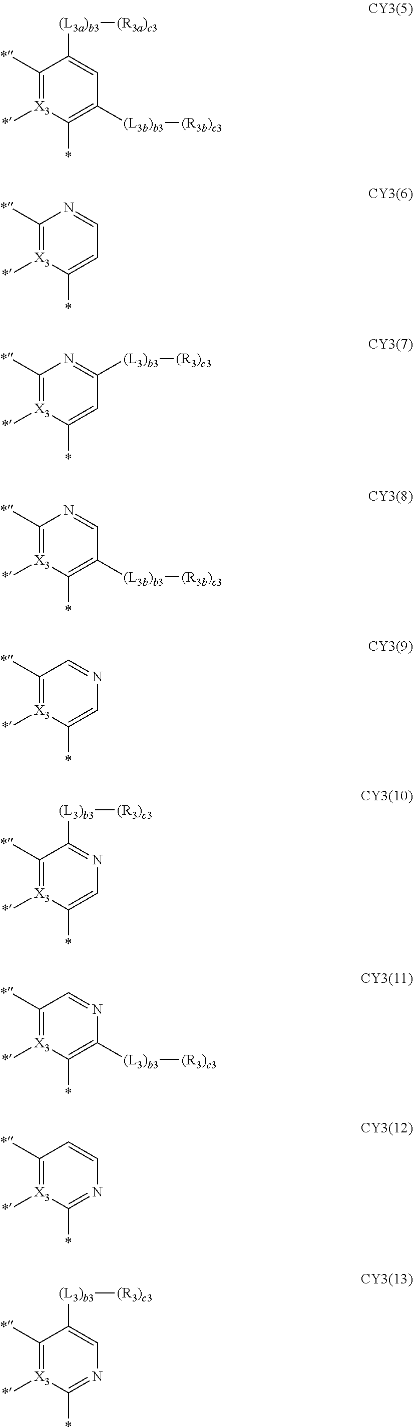

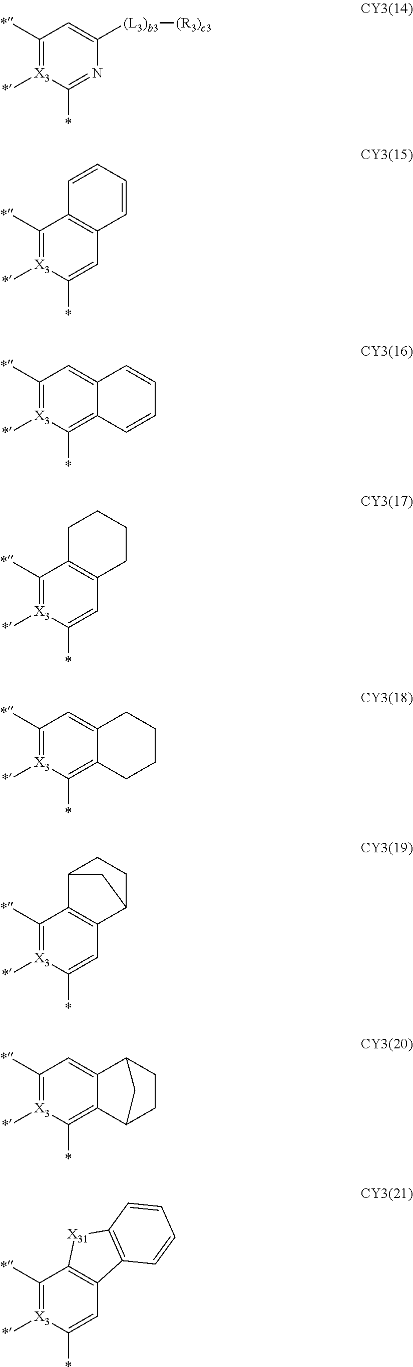

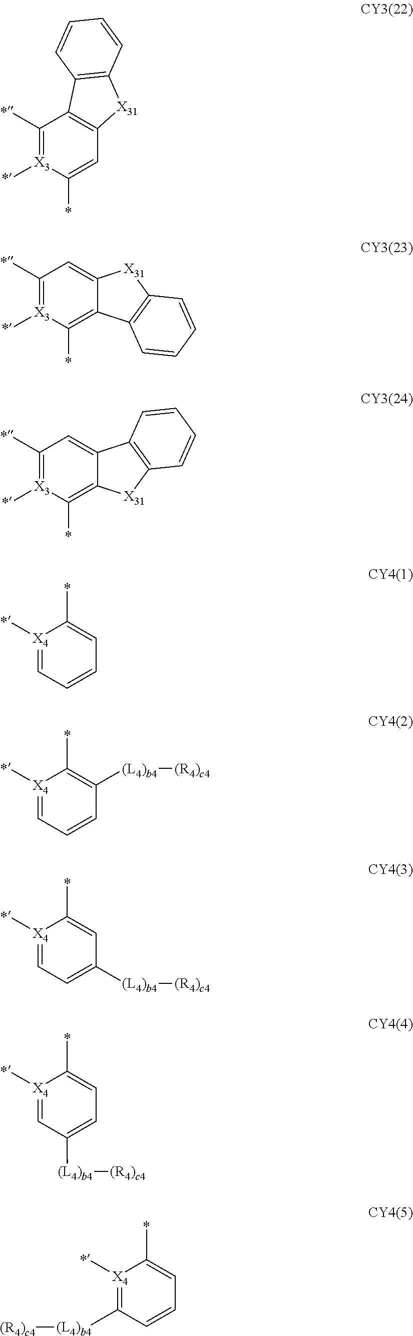

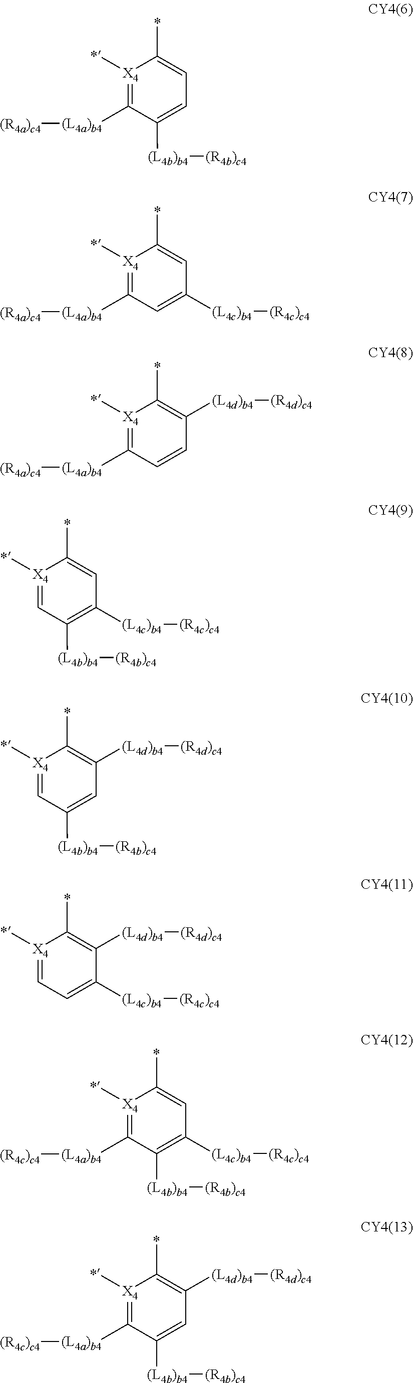

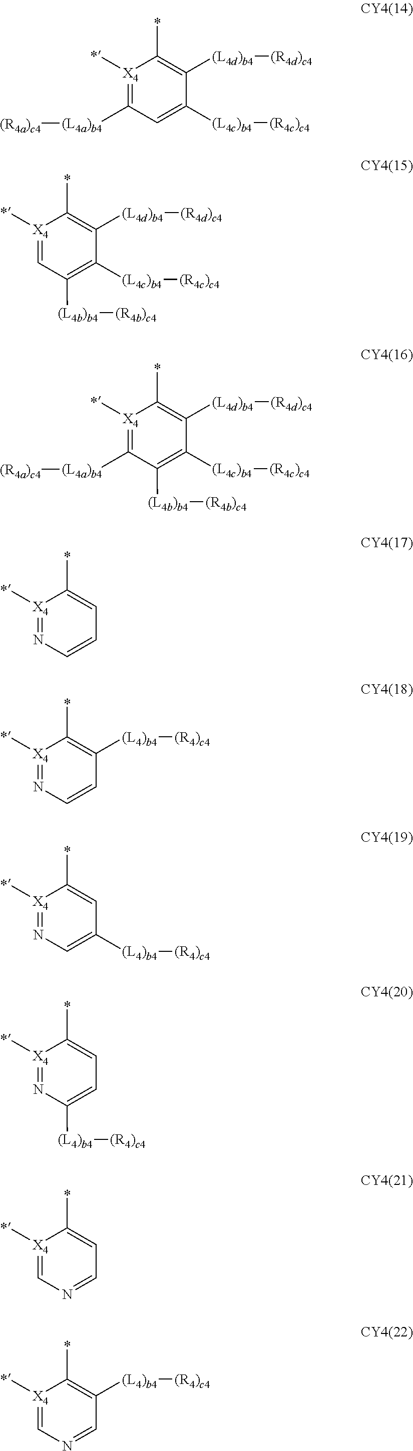

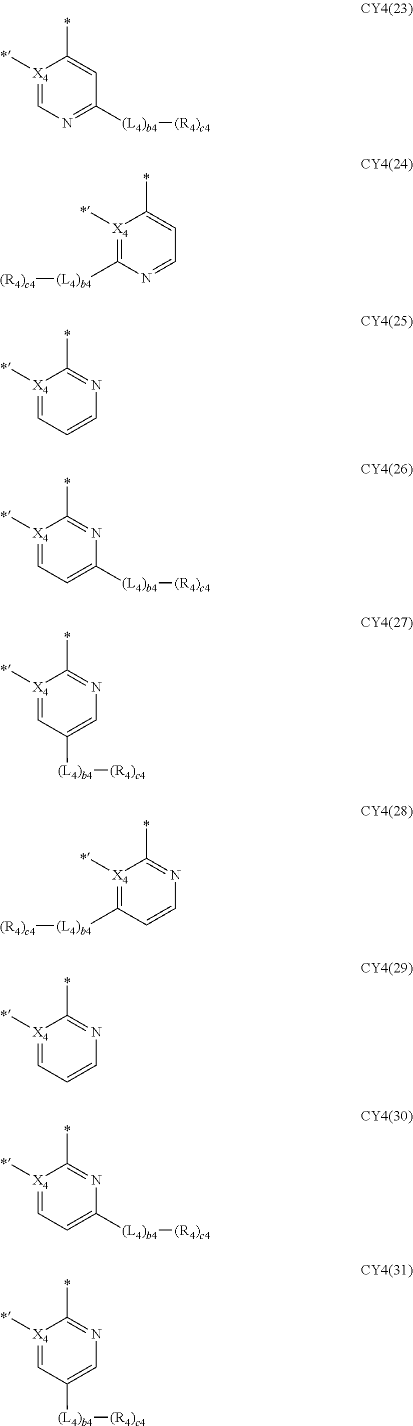

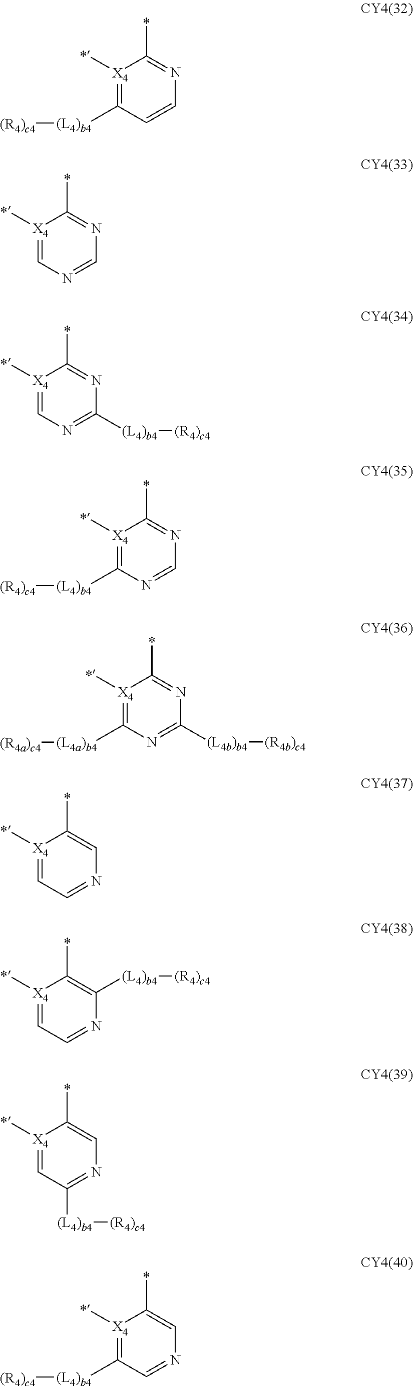

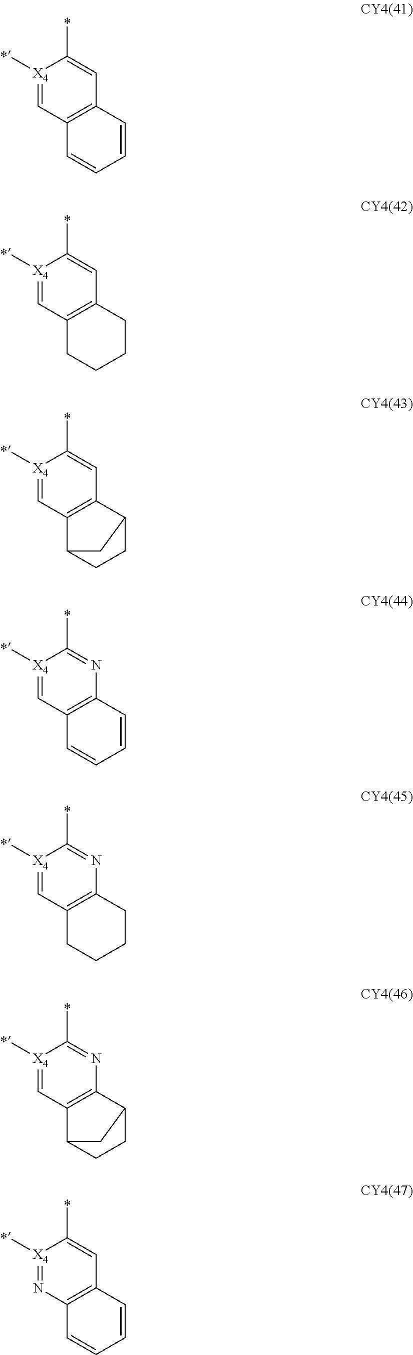

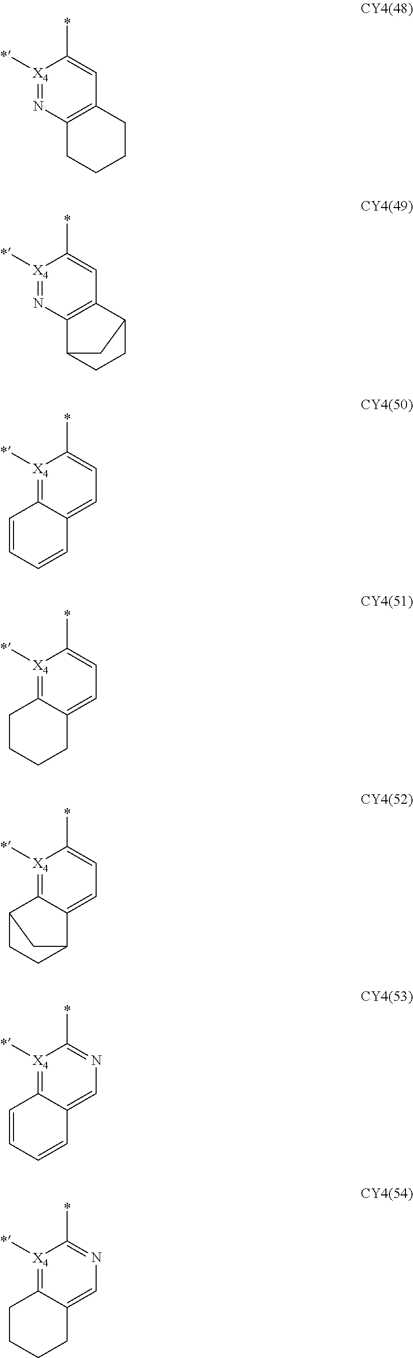

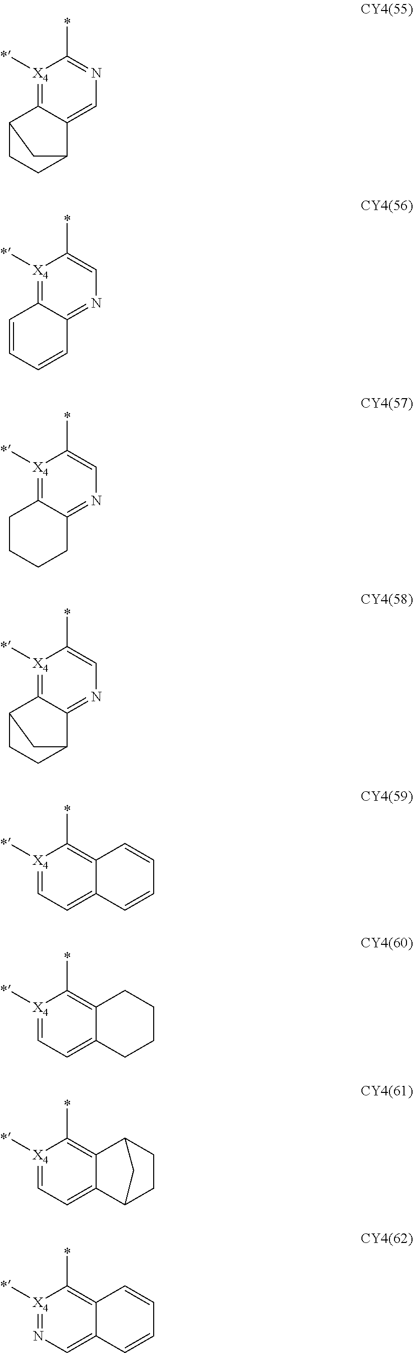

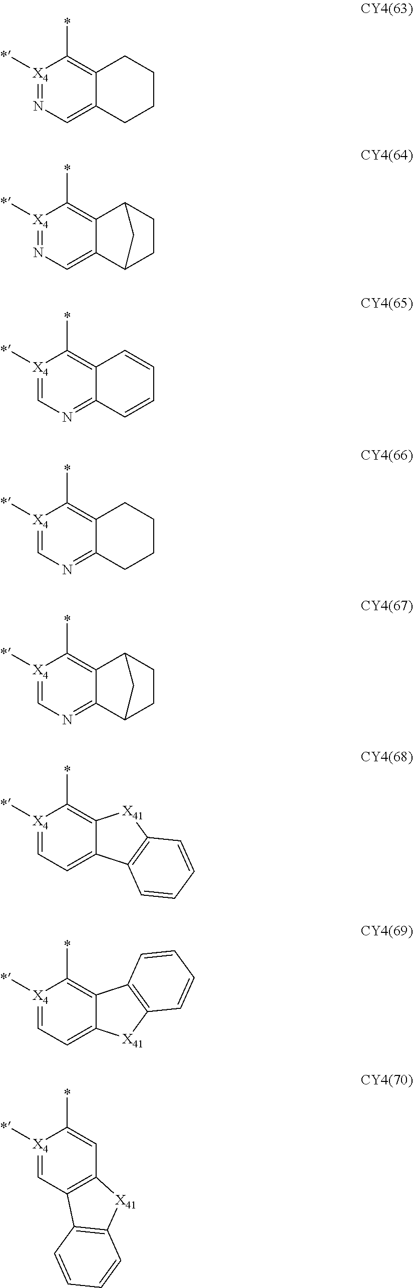

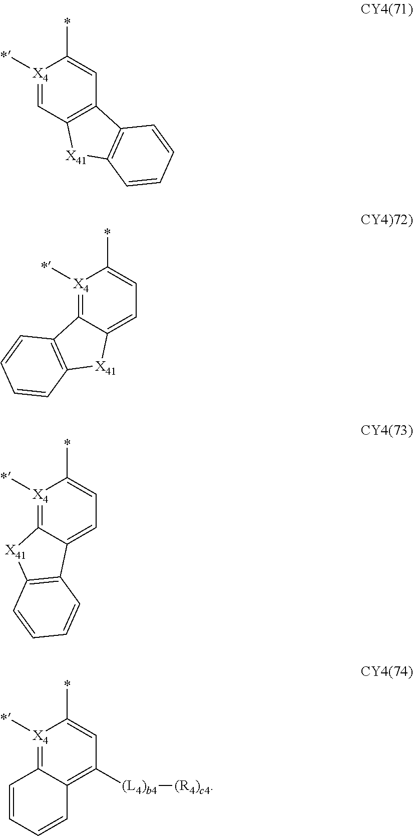

In Formulae CY1(1) to CY1(8), CY2(1) to CY2(4), CY3(1) to CY3(24), and CY4(1) to CY4(74),
X₂ to X₄, Y₁, L₁ to L₄, b1 to b4, R₁ to R₄, and c1 to c4 may each independently be the same as described herein,
X₃₁ may be O, S, N(R₃₁), C(R₃₁)(R₃₂), or Si(R₃₁)(R₃₂),
X₄₁ may be O, S, N(R₄₁), C(R₄₁)(R₄₂), or Si(R₄₁)(R₄₂),
L₁ₐ and L_{1b} may each independently be the same as described in connection with L₁,
R₁ₐ and R_{1b} may each independently be the same as described in connection with R₁,
L₃ₐ and L_{3b} may each independently be the same as described in connection with L₃,
R₃ₐ, R_{3b}, R₃₁, and R₃₂ may each independently be the same as described in connection with R₃,
L₄ₐ to L_{4d} may each independently be the same as described in connection with L₄,
R₄ₐ to R_{4d}, R₄₁, and R₄₂ may each independently be the same as described in connection with R₄,
*-(L₁)_{b1}-(R₁)_{c1}, *-(L₁ₐ)_{b1}-(R₁ₐ)_{c1}, *-(L_{1b})_{b1}-(R₁ₐ)_{c1}, *-(L₂)_{b2}-(R₂)_{c2}, *-(L₃)_{b3}-(R₃)_{c3}, * (L₃ₐ)_{b3}-(R₃ₐ)_{c3}, *-(L_{3b})_{b3}-(R₃ₐ)_{c3}, *-(L₄)_{b4}-(R₄)_{c4}, *-(L₄ₐ)_{b4}-(R₄ₐ)_{c4}, *-(L_{4b})_{b4}-(R₄ₐ)_{c4}, *-(L_{4c})_{b4}-(R_{4c})_{c4}, and *-(L_{4d})_{b4}-(R_{4d})_{c4} may not be hydrogen, and * indicates a binding site to a neighboring atom,
in Formulae CY1(1) to CY1(8), *′ indicates a binding site to X₁ in Formula 1,
in Formulae CY2(1) to CY2(4), CY3(1) to CY3(24), and CY4(1) to CY4(74), *′ indicates a binding site to M in Formula 1,
in Formulae CY1(1) to CY1(8), * indicates a binding site to Y₃ in Formula 1,
in Formulae CY2(1) to CY2(4), * indicates a binding site to ring CY₁ in Formula 1, and *″ indicates a binding site to ring CY₃ in Formula 1,
in Formulae CY3(1) to CY3(24), *″ indicates a binding site to ring CY₂ in Formula 1, and * indicates a binding site to T₁ in Formula 1, and
in Formulae CY4(1) to CY4(74), * indicates a binding site to T₁ in Formula 1.

In an embodiment, a group represented by

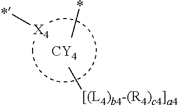

may be a group represented by Formula CY4-1 or CY4-2:

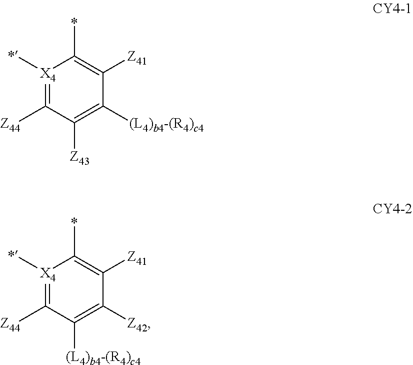

in Formulae CY4-1 and CY4-2,
X₄, L₄, b4, R₄, and c4 may each independently be the same as described herein,
Z₄₁ to Z₄₄ may each independently be the same as described in connection with R₄ herein,
*′ indicates a binding site to M in Formula 1, and
indicates a binding site to T₁ in Formula 1.

For example, Formulae CY4-1 and CY4-2 may satisfy:

1) Condition 1, Condition 2, or any combination thereof (or, Condition 1(1), Condition 2(1), or any combination thereof), and/or

2) Condition 3, Condition 4, Condition 5, or any combination thereof (or, Condition 3(1), Condition 4(1), Condition 5(1), or any combination thereof).

In an embodiment, in Formula 1, a group represented by

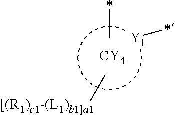

may be a group represented by Formula CY1(1) or CY1(6), and/or

a group represented by

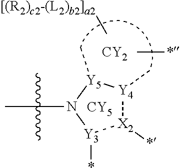

may be a group represented by Formula CY2(1), and/or

a group represented by

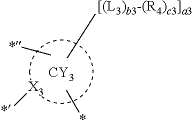

may be a group represented by Formula CY3(3), and/or
a group represented by

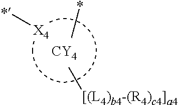

may be a group represented by Formula CY4(3), CY4(4) or CY4(16).

In an embodiment, a group represented by

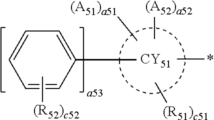

in Formula 1 may be a group represented by one of Formulae 51-1 to 51-32:

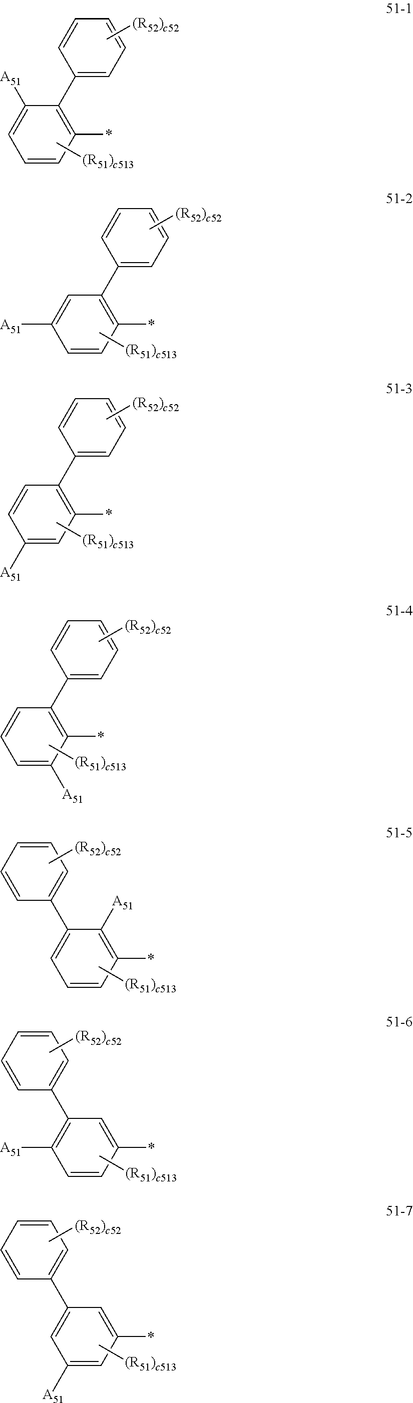

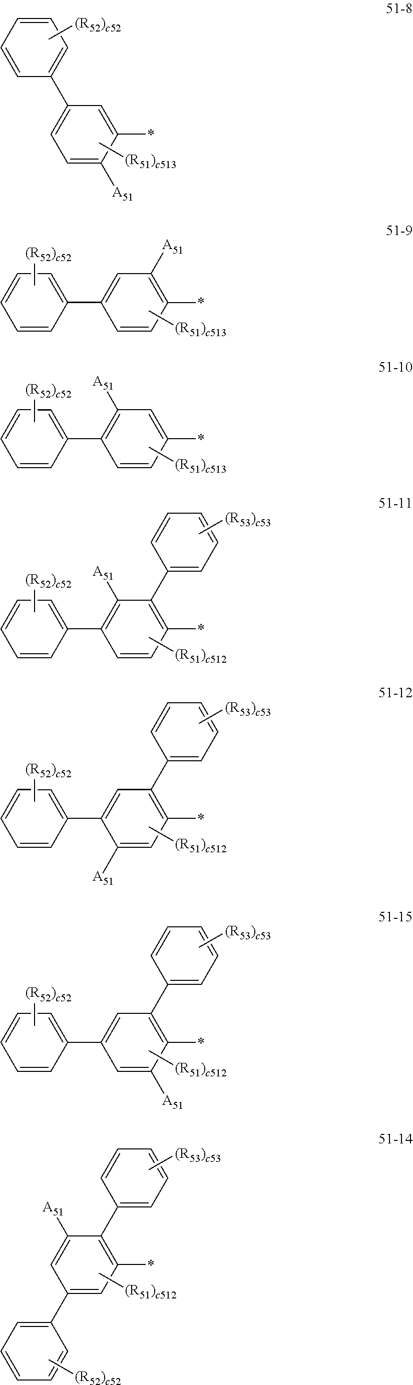

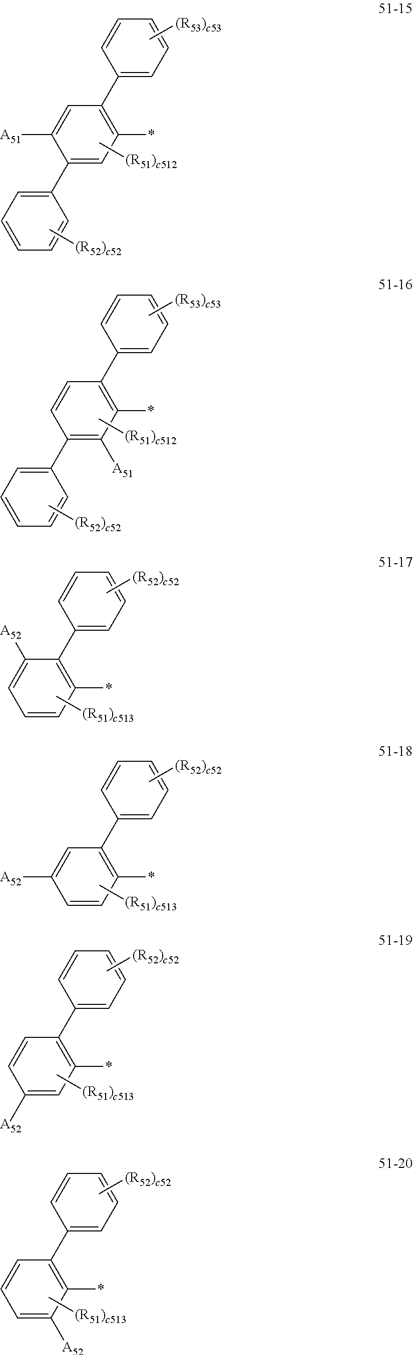

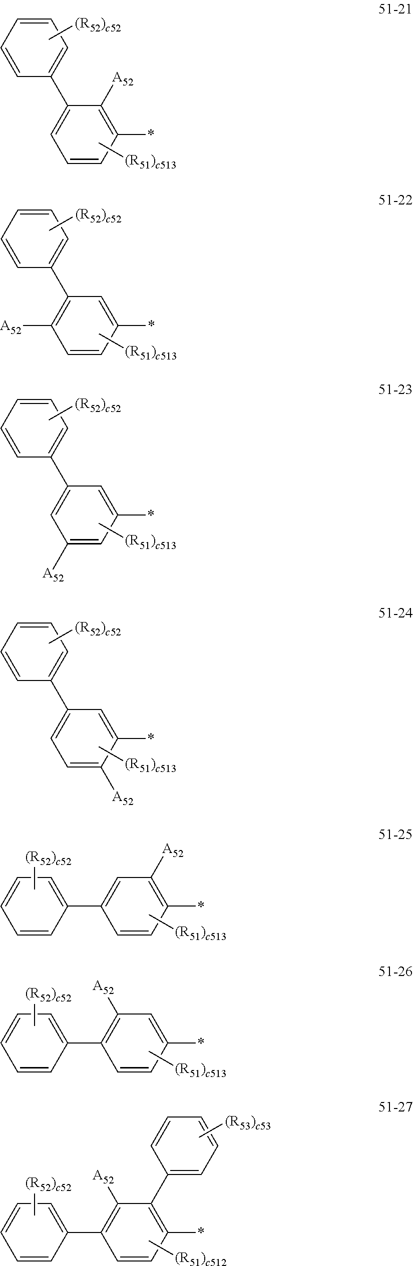

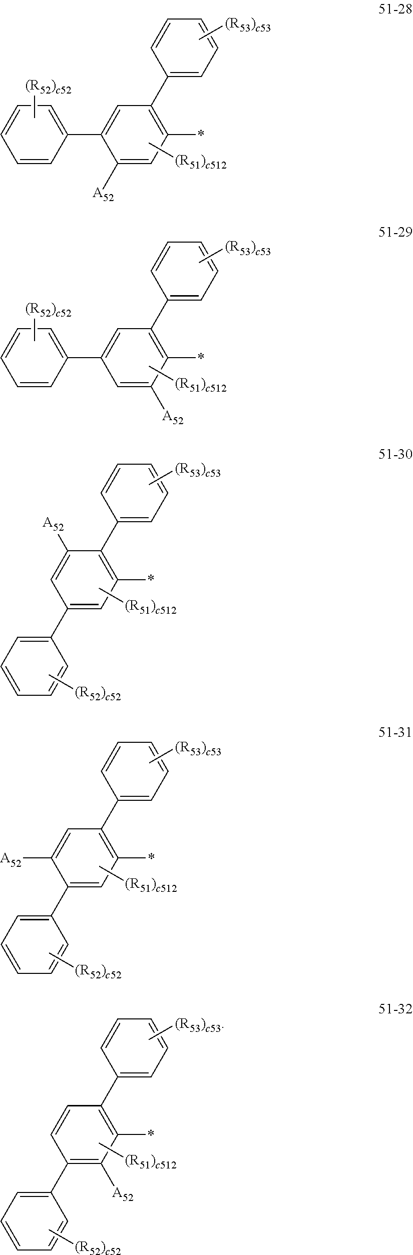

In Formulae 51-1 to 51-32, R₅₁, R₅₂, c52, A₅₁, and A₅₂ may each independently be the same as described herein, R₅₃ and c53 may each independently be the same as described in connection with R₅₂ and c52, c512 is an integer of 0 to 2, c513 is an integer of 0 to 3, and * indicates a binding site to L₅₁.

In an embodiment, the organometallic compound may be represented by Formula 1-1 or 1-2:

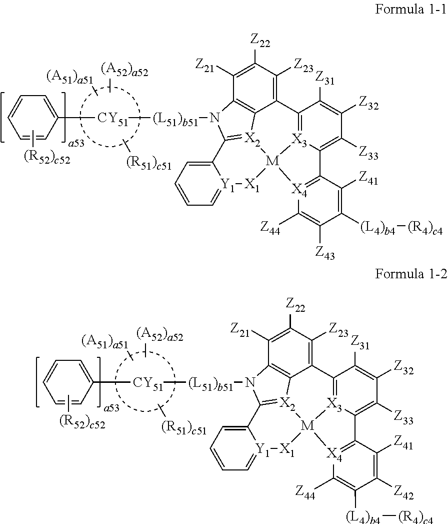

in Formulae 1-1 and 1-2,

M, X₁ to X₄, Y₁, Y₃ to Y₅, L₄, L₅₁, b4, b51, R₅₁, R₅₂, c51, c52, A₅₁, A₅₂, a51, a52 and a53 may each independently be the same as described herein,

Z₁₁ to Z₁₄ may each independently be the same as described in connection with R₁,

Z₂₁ to Z₂₃ may each independently be the same as described in connection with R₂,

Z₃₁ to Z₃₃ may each independently be the same as described in connection with R₃,

Z₄₁ to Z₄₄ may each independently be the same as described in connection with R₄,

two or more groups of Z₁₁ to Z₁₄ may be optionally linked to form a C₅-C₃₀ carbocyclic group that is unsubstituted or substituted with an R₁₀ₐ group or a C₂-C₃₀ heterocyclic group that is unsubstituted or substituted with an R₁₀ₐ group,

two or more groups of Z₂₁ to Z₂₃ may be optionally linked to form a C₅-C₃₀ carbocyclic group that is unsubstituted or substituted with an R₁₀ₐ group or a C₂-C₃₀ heterocyclic group that is unsubstituted or substituted with an R₁₀ₐ group,

two or more groups of Z₃₁ to Z₃₃ may be optionally linked to form a C₅-C₃₀ carbocyclic group that is unsubstituted or substituted with an R₁₀ₐ group or a C₂-C₃₀ heterocyclic group that is unsubstituted or substituted with an R₁₀ₐ group,

two or more groups of Z₄₁ to Z₄₄ may be optionally linked to form a C₅-C₃₀ carbocyclic group that is unsubstituted or substituted with an R₁₀ₐ group or a C₂-C₃₀ heterocyclic group that is unsubstituted or substituted with an R₁₀ₐ group, and

R₁₀ₐ is the same as described in connection with R₁.

Descriptions for Formula 1 described herein can be applied for Formulae 1-1 and 1-2.

For example,

1) Formulae 1-1 and 1-2 may satisfy Condition A, Condition B, or any combination thereof (or, Condition A, Condition B(1), or any combination thereof), and/or

2) Formulae 1-1 and 1-2 may satisfy Condition 1, Condition 2, or any combination thereof (or, Condition 1(1), Condition 2(1), or any combination thereof), and/or

3) Formulae 1-1 and 1-2 may satisfy Condition 3, Condition 4, Condition 5, or any combination thereof (or, Condition 3(1), Condition 4(1), Condition 5(1), or any combination thereof), and/or

4) a group represented by

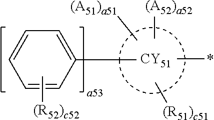

in Formulae 1-1 and 1-2 may be a group represented by one of Formulae 51-1 to 51-32.

In an embodiment, Z₁₂, Z₁₄, Z₂₁ to Z₂₃, Z₃₁, Z₃₃ and Z₄₁ to Z₄₄ in Formulae 1-1 and 1-2 may each independently be hydrogen, deuterium, —CH₃, or —CD₃.

In an embodiment, Z₁₂, Z₁₄, Z₂₁ to Z₂₃, Z₃₁, Z₃₃ and Z₄₁ to Z₄₄ in Formulae 1-1 and 1-2 may each independently be hydrogen, or deuterium.

In an embodiment, Z₁₁ and Z₁₃ in Formulae 1-1 and 1-2 may not be hydrogen and Z₁₁ and Z₁₃ may include a deuterium.

In an embodiment, Z₁₁ and Z₁₃ in Formulae 1-1 and 1-2 may not be hydrogen and Z₁₁ and Z₁₃ may be identical to each other.

In an embodiment, Z₁₁ and Z₁₃ in Formulae 1-1 and 1-2 may not be hydrogen and Z₁₁ and Z₁₃ may be different from each other.

In Formula 1, i) two or more groups of a plurality of R₁ groups may optionally be linked to form a C₅-C₃₀ carbocyclic group that is unsubstituted or substituted with an R₁₀ₐ group or a C₂-C₃₀ heterocyclic group that is unsubstituted or substituted with an R₁₀ₐ group, ii) two or more groups of a plurality of R₂ groups may optionally be linked to form a C₅-C₃₀ carbocyclic group that is unsubstituted or substituted with an R₁₀ₐ group or a C₂-C₃₀ heterocyclic group that is unsubstituted or substituted with an R₁₀ₐ group, iii) two or more groups of a plurality of R₃ groups may optionally be linked to form a C₅-C₃₀ carbocyclic group that is unsubstituted or substituted with an R₁₀ₐ group or a C₂-C₃₀ heterocyclic group that is unsubstituted or substituted with an R₁₀ₐ group, iv) two or more groups of a plurality of R₄ groups may optionally be linked to form a C₅-C₃₀ carbocyclic group that is unsubstituted or substituted with an R₁₀ₐ group or a C₂-C₃₀ heterocyclic group that is unsubstituted or substituted with an R₁₀ₐ group, and v) two or more groups of R₁ to R₆ may optionally be linked to form a C₅-C₃₀ carbocyclic group that is unsubstituted or substituted with an R₁₀ₐ group or a C₂-C₃₀ heterocyclic group that is unsubstituted or substituted with an R₁₀ₐ group. An R₁₀ₐ group may be the same as described in connection with R₁.

For example, a C₅-C₃₀ carbocyclic group (that is unsubstituted or substituted with an R₁₀ₐ group) and a C₂-C₃₀ heterocyclic group (that is unsubstituted or substituted with an R₁₀ₐ group) may each independently be a cyclopentane group, a silole group, an azasilole group, a diazasilole group, a triazasilole group, an adamantane group, a norbornene group, a bicyclo[1.1.1]pentane group, a bicyclo[2.1.1]hexane group, a bicyclo[2.2.1]heptane group, a bicyclo[2.2.2]octane group, a cyclohexane group, a cyclohexene group, a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene-5-oxide group, a 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with an R₁ₐ group, but embodiments of the present disclosure are not limited thereto.

Non-limiting examples of the C₁-C₆₀ alkyl group, C₁-C₂₀ alkyl group and/or C₁-C₁₀ alkyl group include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group, each unsubstituted or substituted with a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, or any combination thereof, and the like, but embodiments of the present disclosure are not limited thereto.

Non-limiting examples of the C₁-C₆₀ alkoxy group, C₁-C₂₀ alkoxy group and/or C₁-C₁₀ alkoxy group include a methoxy group, an ethoxy group, a propoxy group or a butoxy group, and the like, but embodiments of the present disclosure are not limited thereto.

Non-limiting examples of the C₃-C₁₀ cycloalkyl group include a cyclopropyl group, a cyclobutyl group, a cyclopentyl group, a cyclohexyl group, a cycloheptyl group, a cyclooctyl group, an adamantyl group, a bicyclo[1.1.1]pentyl group, a bicyclo[2.1.1]hexyl group, a bicyclo[2.2.1]heptyl group, a bicyclo[2.2.2]octyl group, and the like, but embodiments of the present disclosure are not limited thereto.

An angle formed by a plane including the transition dipole moment of the organometallic compound represented by Formula 1 and a plane including four atoms of a tetradentate ligand linked to a metal M of Formula 1 may be about 10° or less. In addition, a horizontal orientation ratio of the transition dipole moment of the organometallic compound represented by Formula 1 may be in a range of about 80% to about 100%.

For example, the angle formed by the plane including the transition dipole moment of the organometallic compound and the plane including the four atoms of the tetradentate ligand linked to the metal (or platinum) of Formula 1 may be 0° to 10°, 0° to 9°, 0° to 8°, 0° to 7°, 0° to 6°, 0° to 5°, 0° to 4°, 0° to 3°, 0° to 2°, or 0° to 1°, but embodiments of the present disclosure are not limited thereto. When the angle formed by the plane including the transition dipole moment of the organometallic compound represented by the Formula 1 and the plane including the four atoms of the tetradentate ligand linked to the metal of Formula 1 is within these ranges, the organometallic compound may have excellent planarity, and a thin film formed by using the organometallic compound may have excellent electric characteristics.

In an embodiment, the horizontal orientation ratio of the transition dipole moment of the organometallic compound may be in a range of, for example, 80% to 100%, 81% to 100%, 82% to 100%, 83% to 100%, 84% to 100%, 85% to 100%, 86% to 100%, 87% to 100%, 88% to 100%, 89% to 100%, 90% to 100%, 91% to 100%, 92% to 100%, 93% to 100%, 94% to 100%, 95% to 100%, 96% to 100%, 97% to 100%, 98% to 100%, 99% to 100%, or 100%, but embodiments of the present disclosure are not limited thereto.

The horizontal orientation ratio of the transition dipole moment means a ratio of the organometallic compound having a transition dipole moment horizontal to the film including the organometallic compound to the total organometallic compound in the film including the organometallic compound.

The horizontal orientation ratio of the transition dipole moment may be evaluated by using an angle-dependent PL measurement apparatus. The angle-dependent PL measurement apparatus may be understood by referring to Korean Patent Application No. 10-2013-0150834, the content of which is incorporated herein in its entirety by reference.

Since the organometallic compound has a high horizontal orientation ratio of a transition dipole moment, the organometallic compound has a large horizontal orientation transition dipole moment (that is, a large horizontal optical orientation). Therefore, a large amount of electric field traveling in a direction perpendicular to the film including the organometallic compound may be emitted. Light emitted due to such a mechanism may have high external extraction efficiency (that is, efficiency of extracting light emitted in the organometallic compound from a device (for example, an organic light-emitting device) including a film (for example, an emission layer described below) including the organometallic compound) to the outside, and thus, an electronic device, for example, an organic light-emitting device, which includes the organometallic compound, may have high luminescent efficiency.

The terms “an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluorene-9-one group, and an azadibenzothiophene 5,5-dioxide group” as used herein each refer to a heterocyclic group having the same backbone as that of each of “an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluorene-9-one group, and a dibenzothiophene 5,5-dioxide group”, respectively, wherein an ring-forming carbons of the rings above is substituted with nitrogen.

In addition, R₁₀ₐ is defined the same as R₁.

For example, the organometallic compound represented by Formula 1 may include a deuterium.

In an embodiment, the organometallic compound may be one of the following Compounds 1 to 3599:

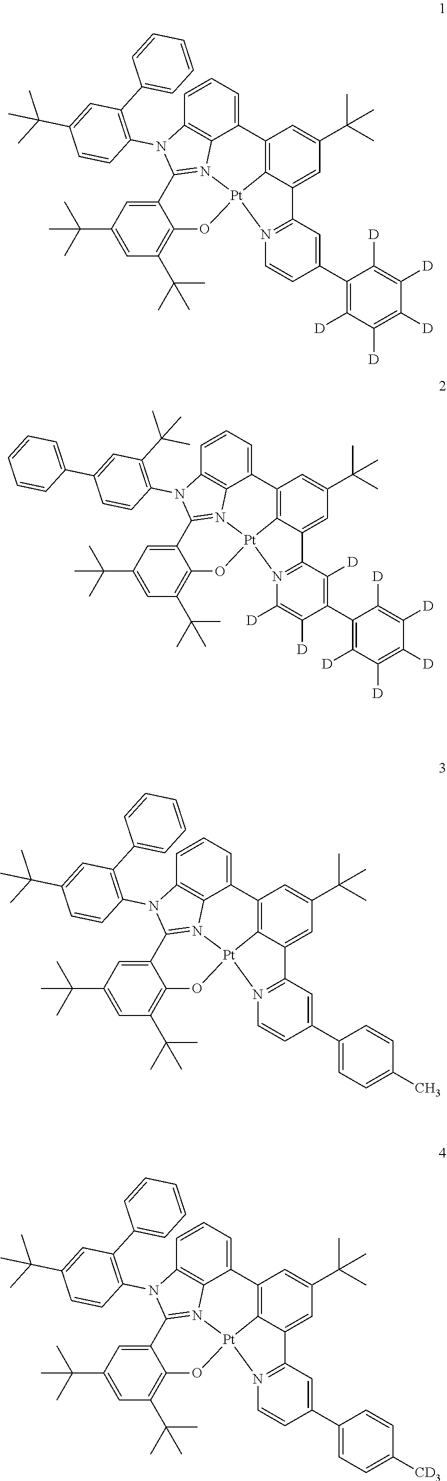

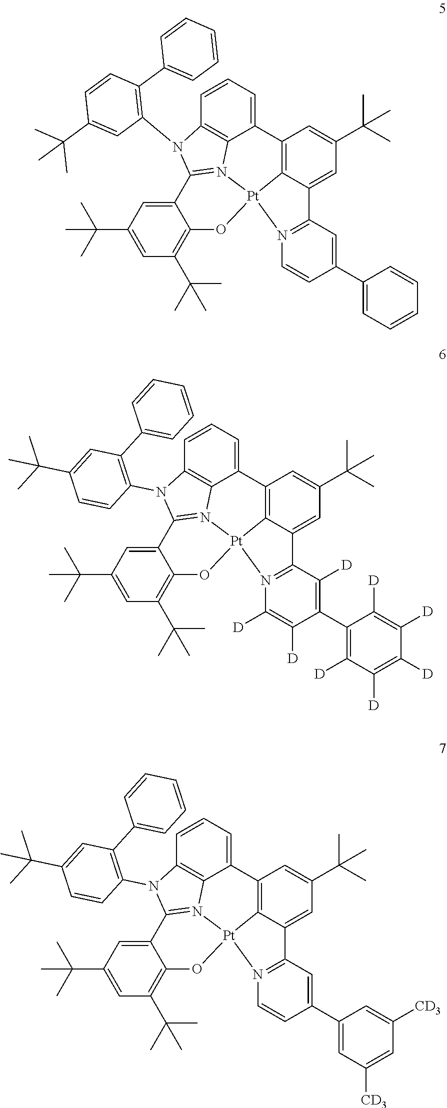

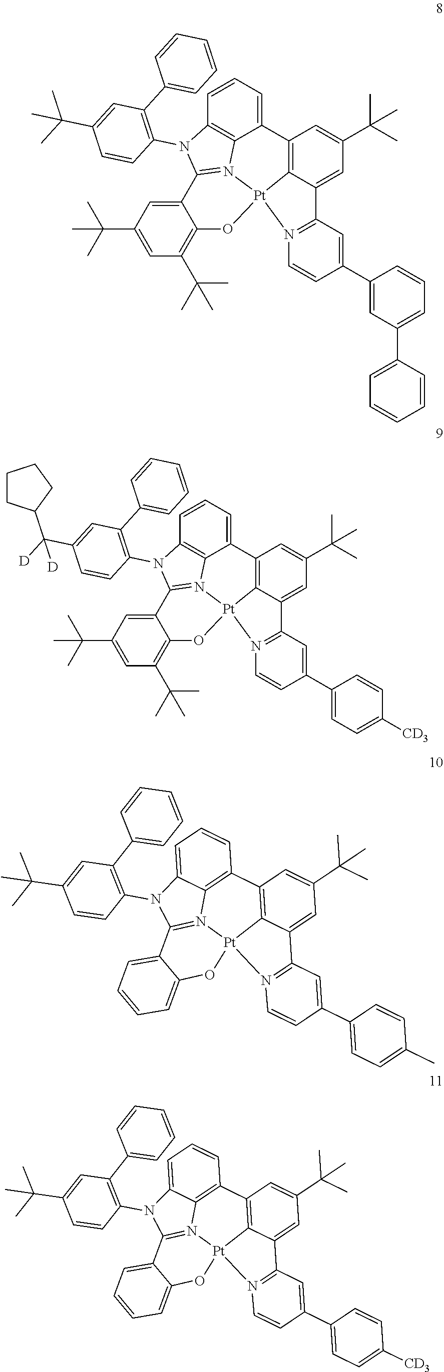

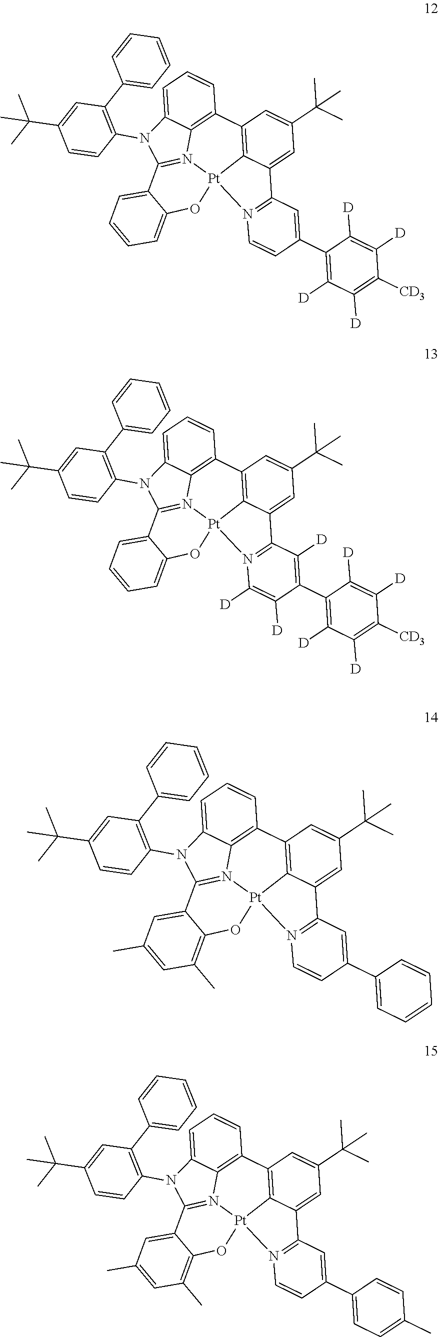

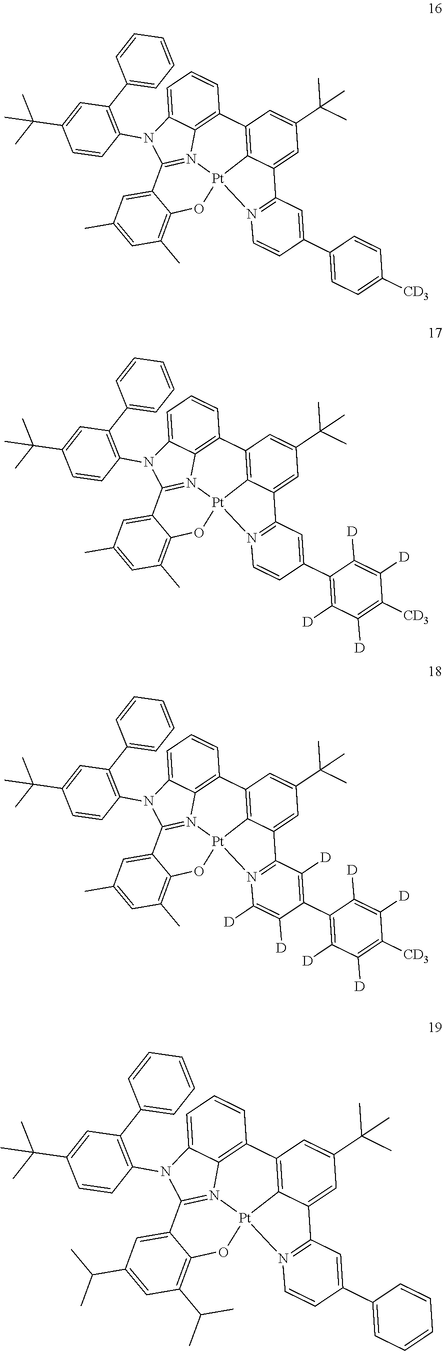

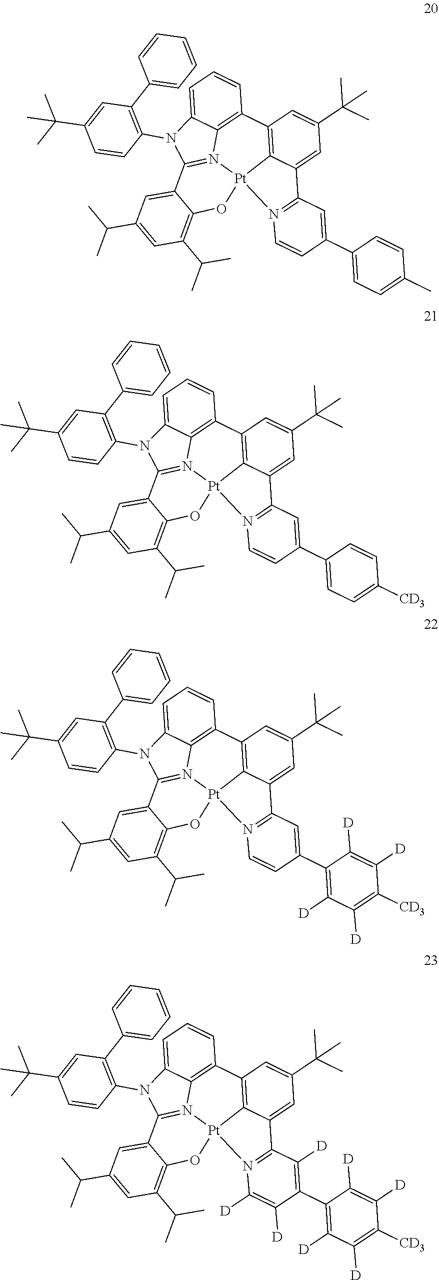

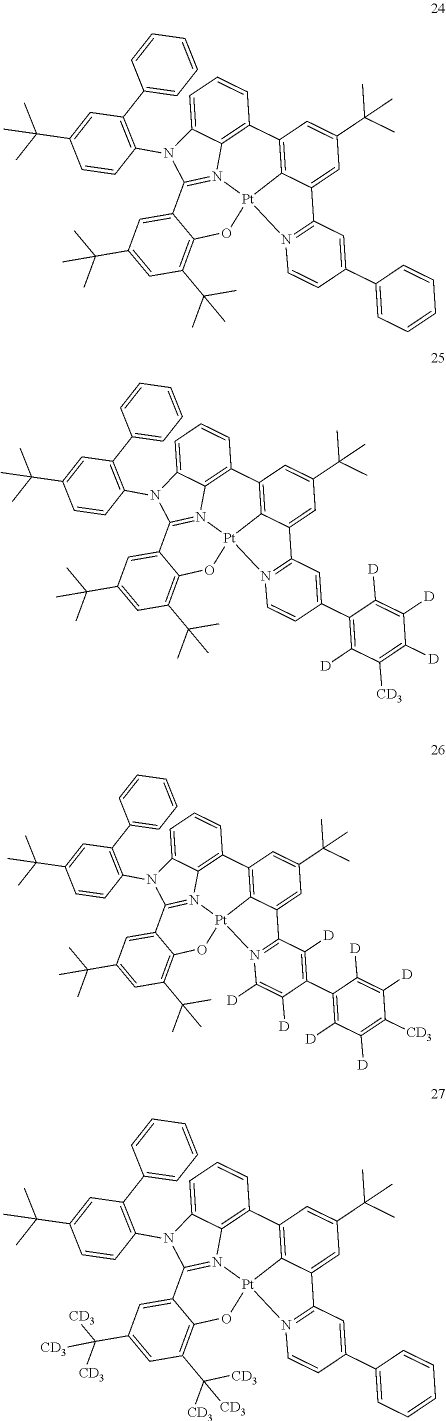

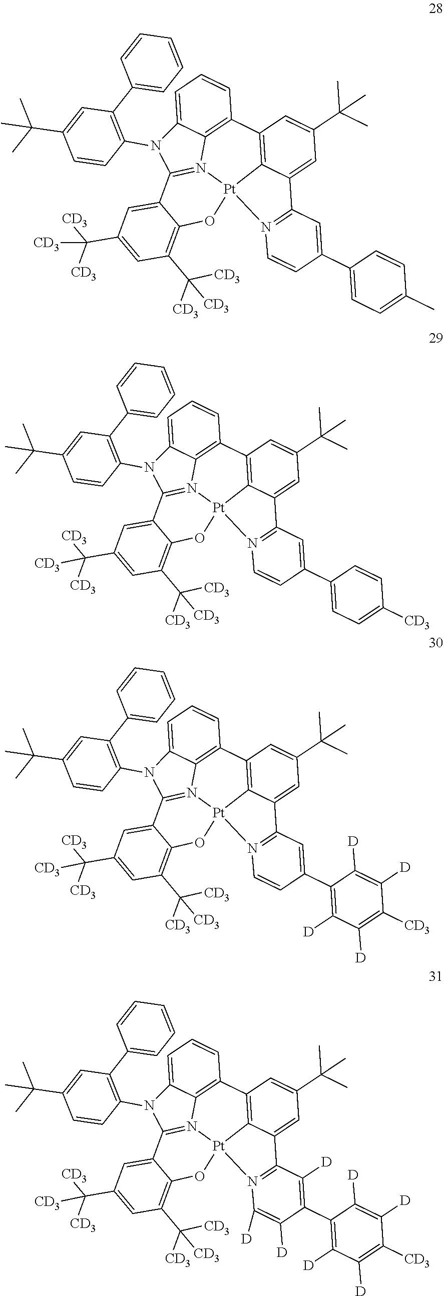

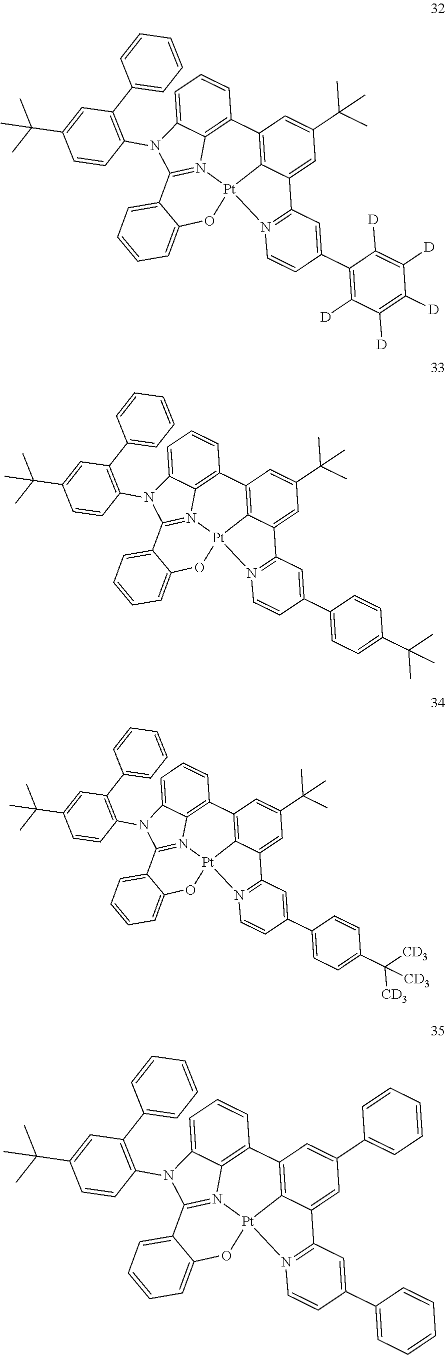

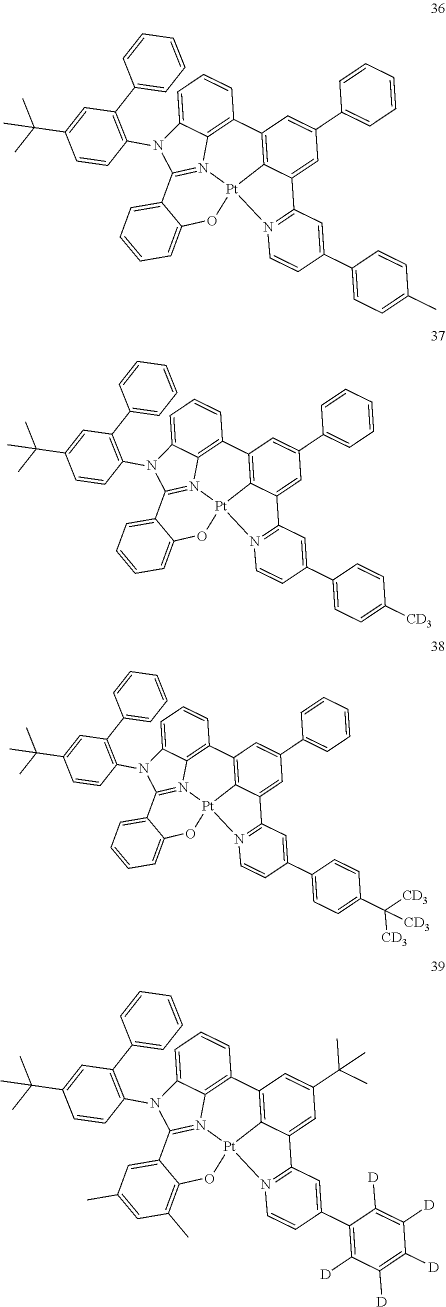

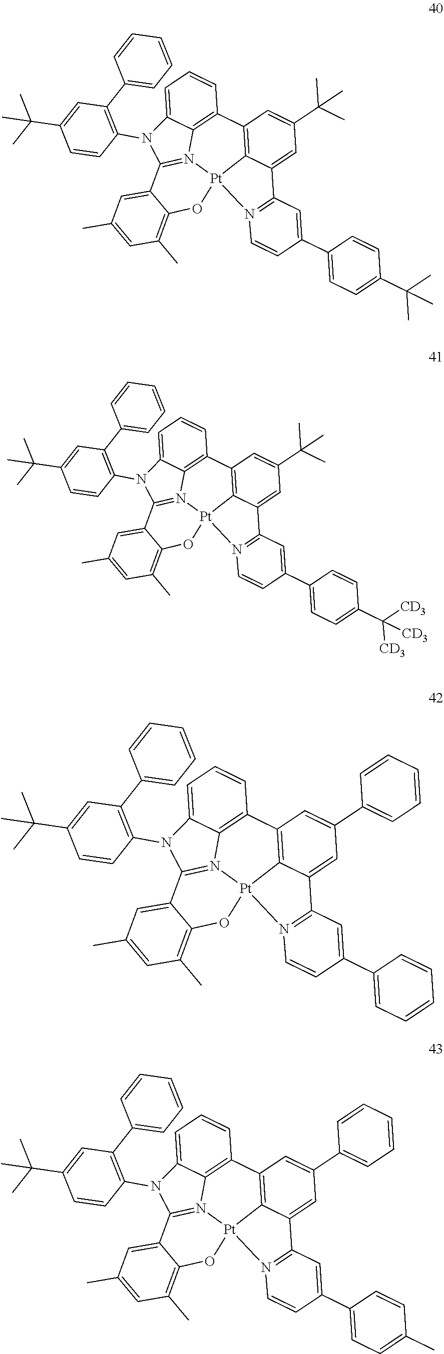

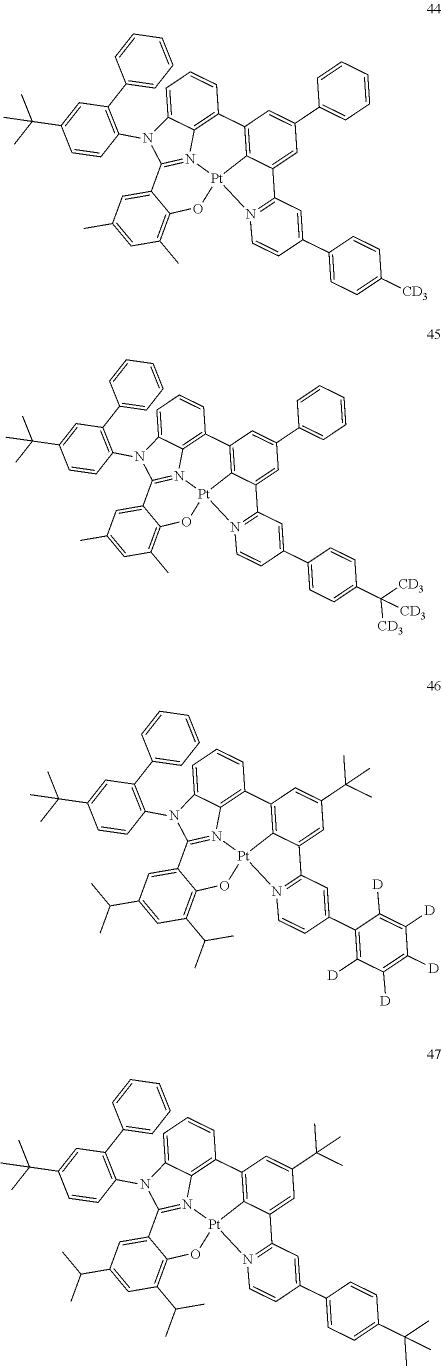

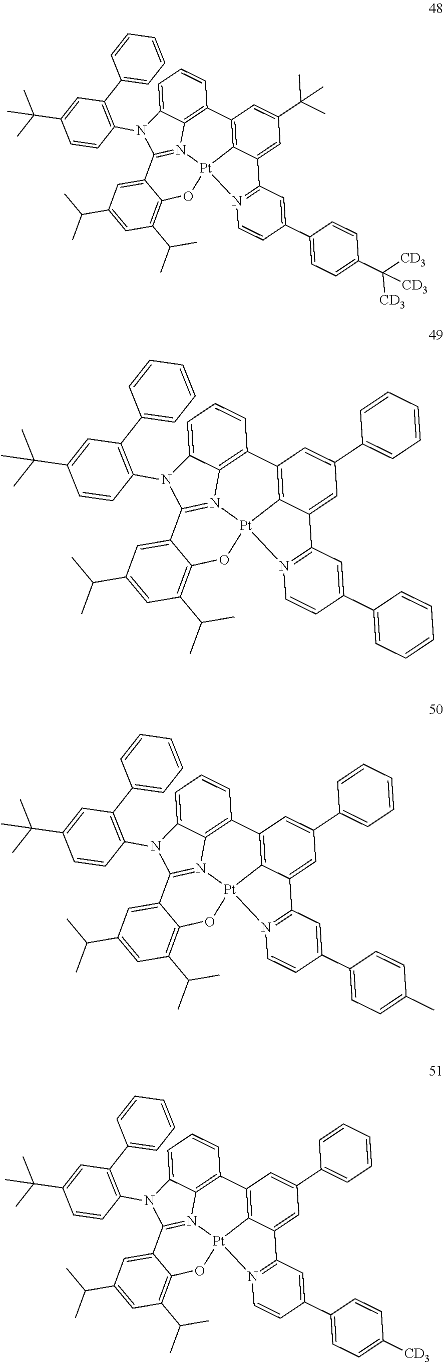

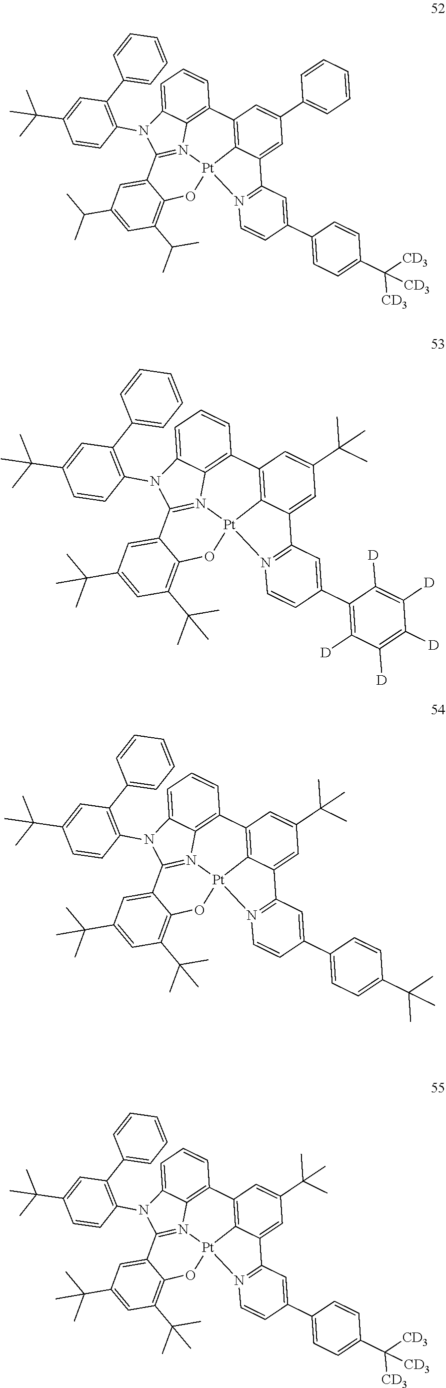

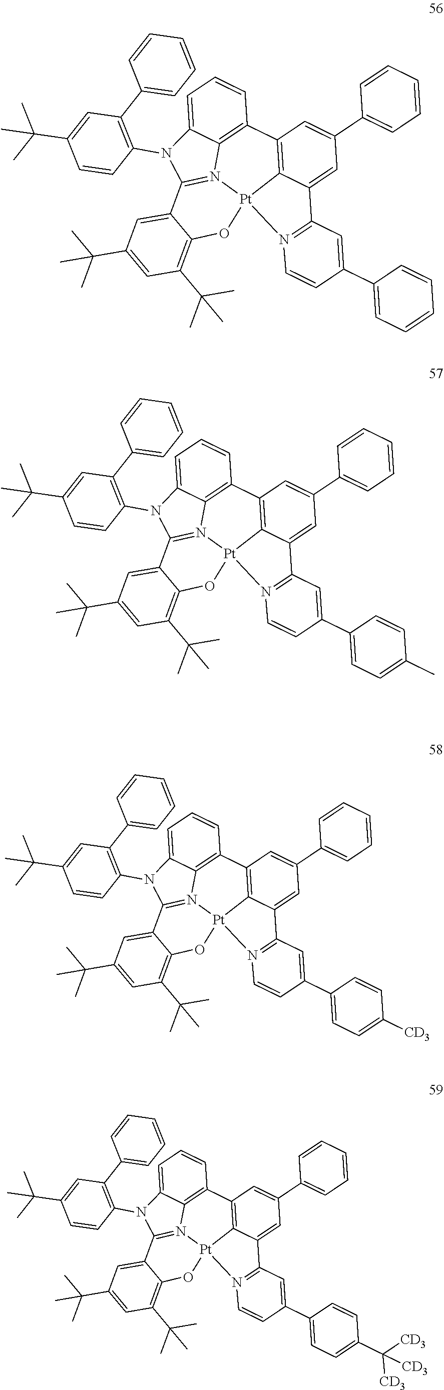

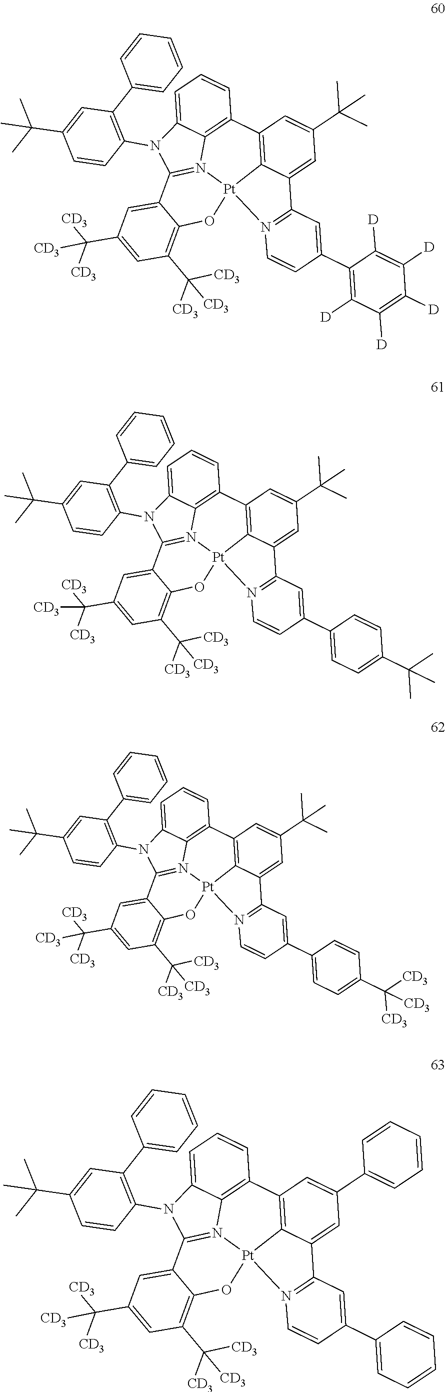

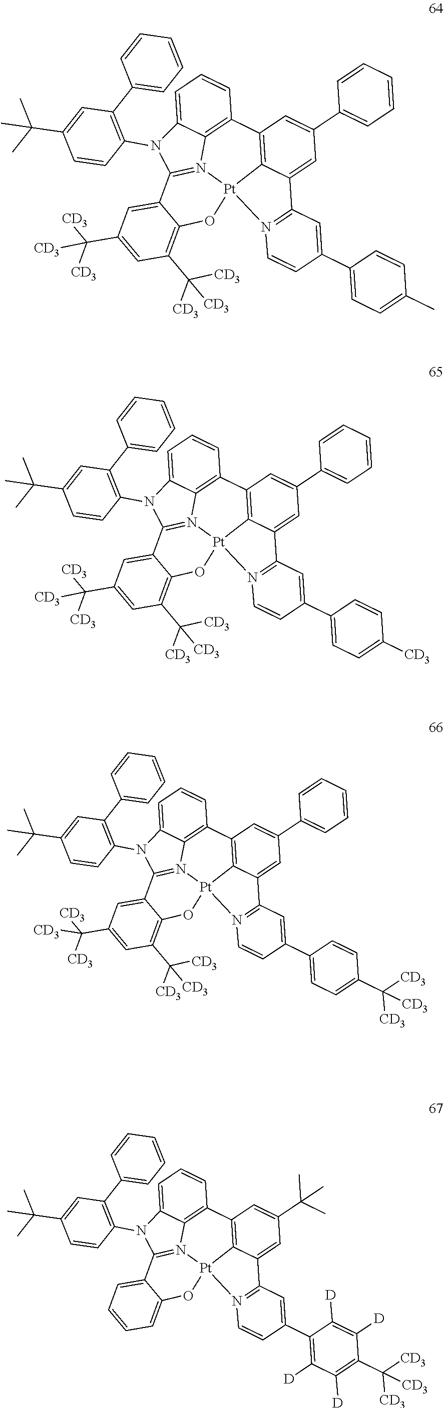

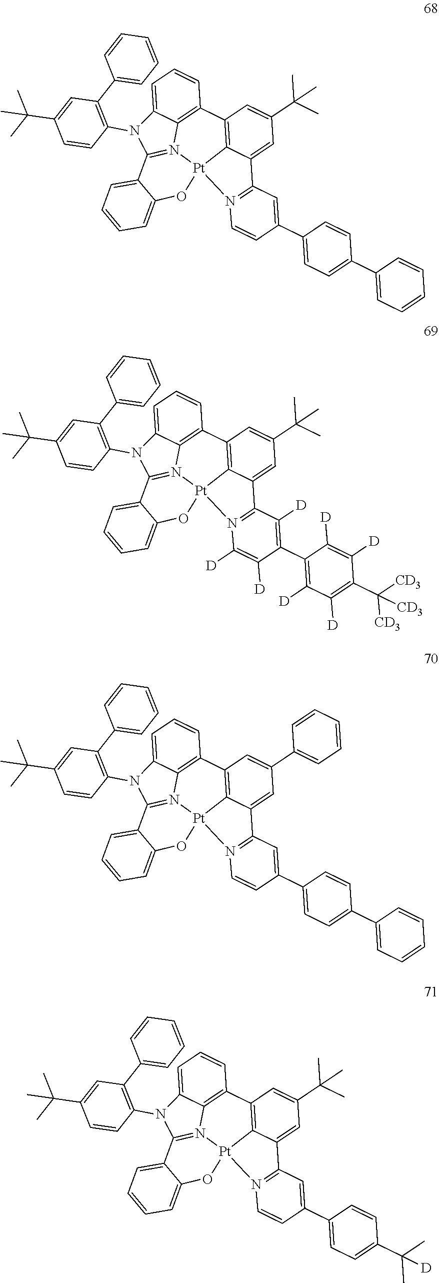

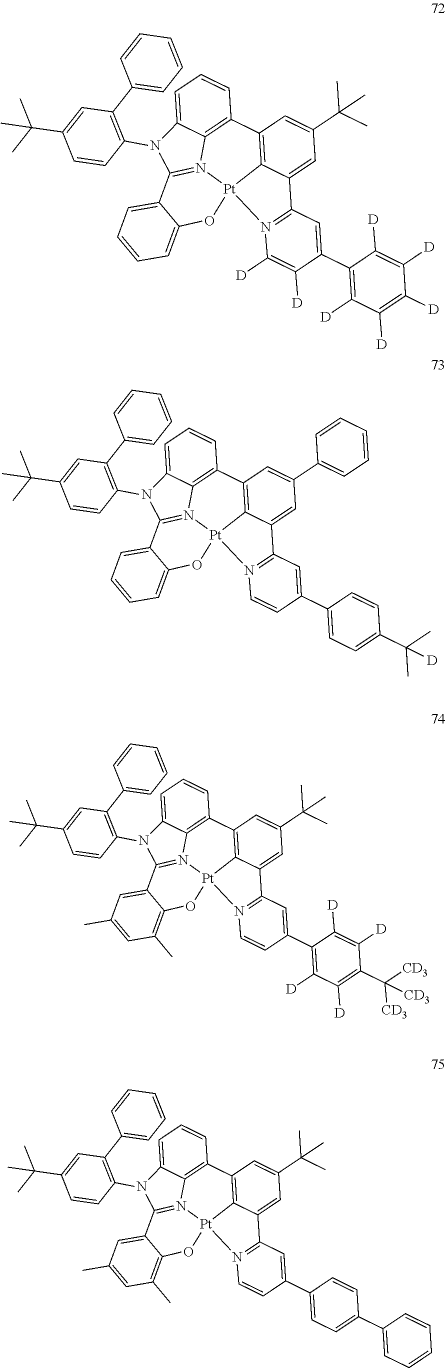

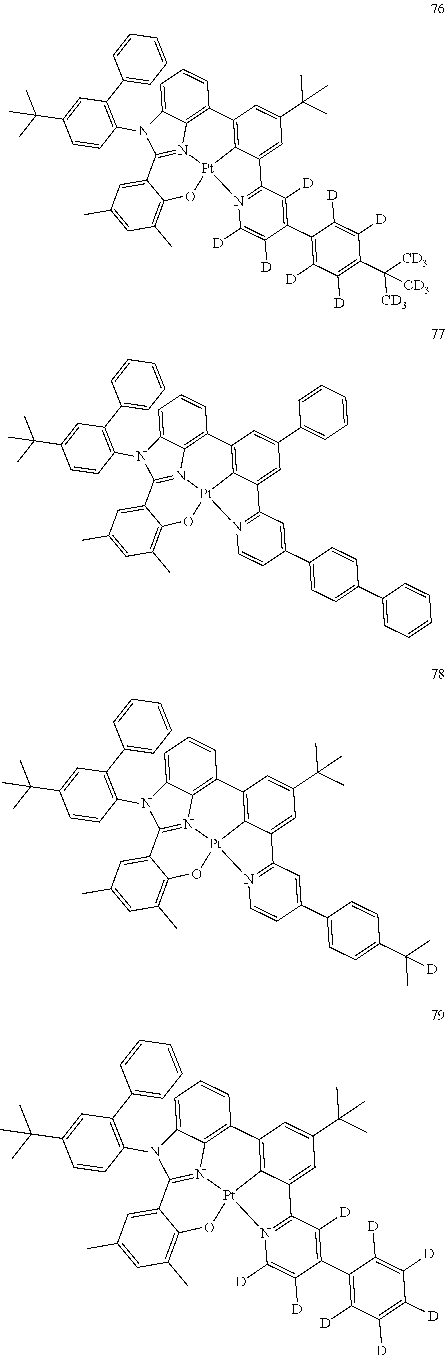

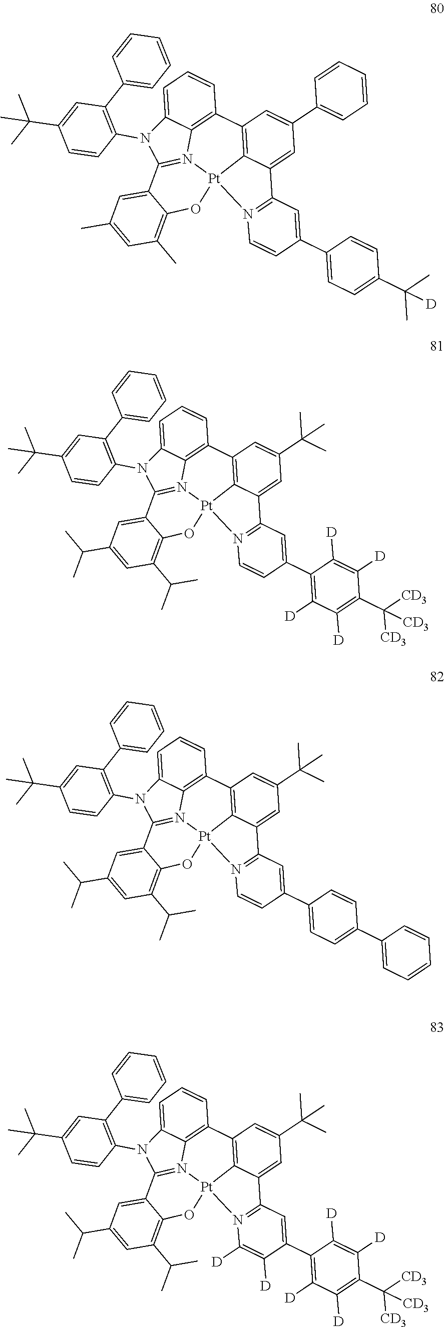

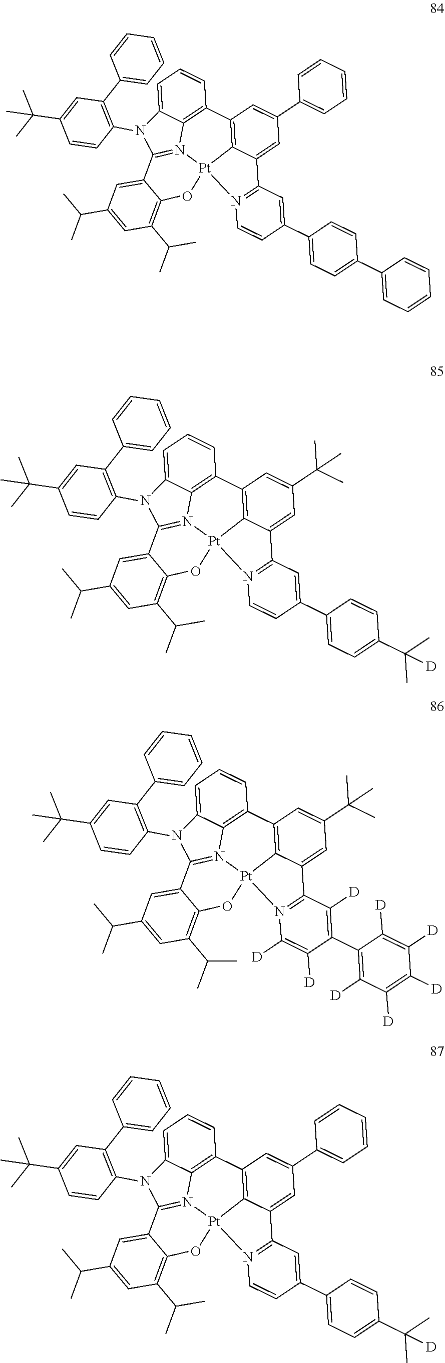

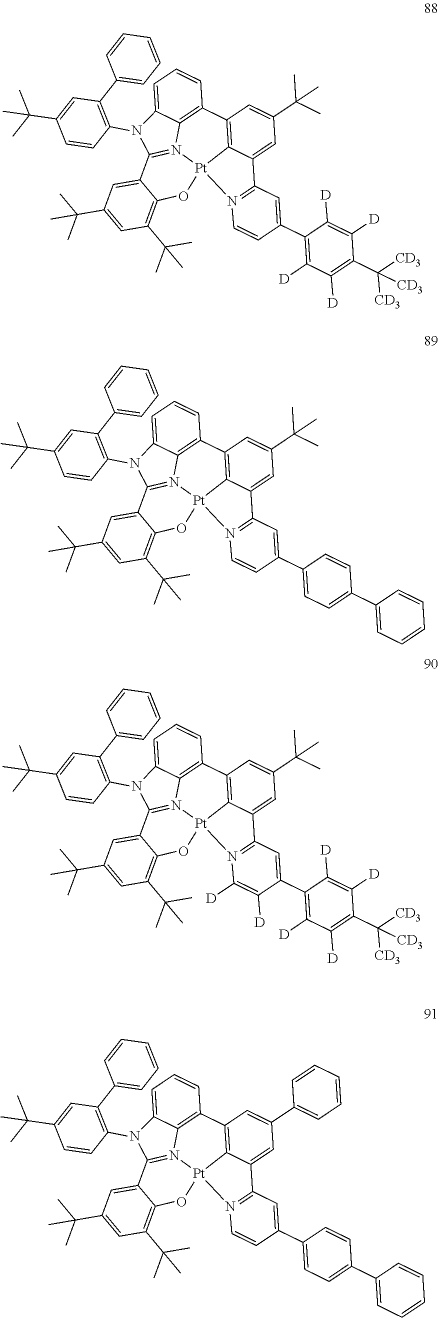

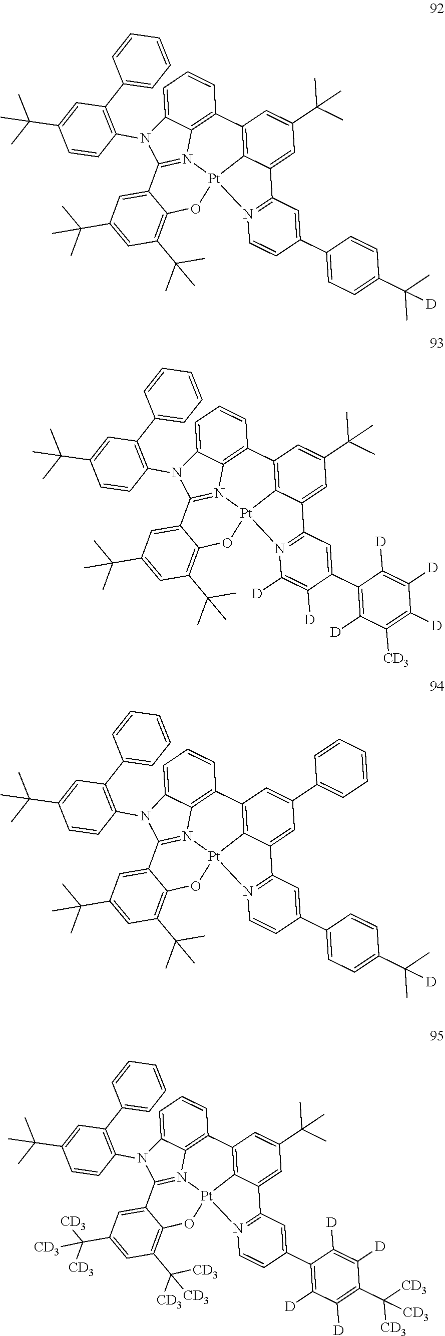

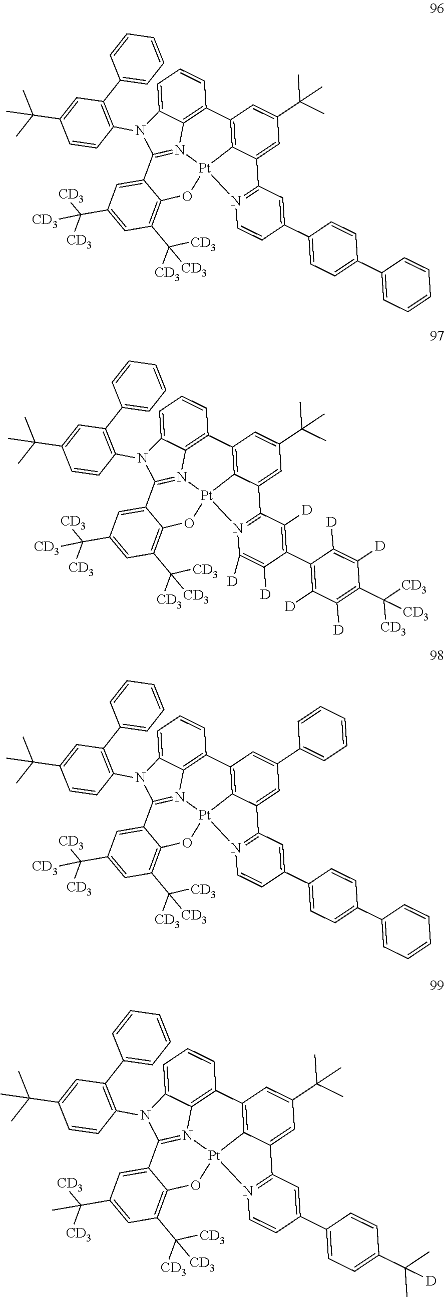

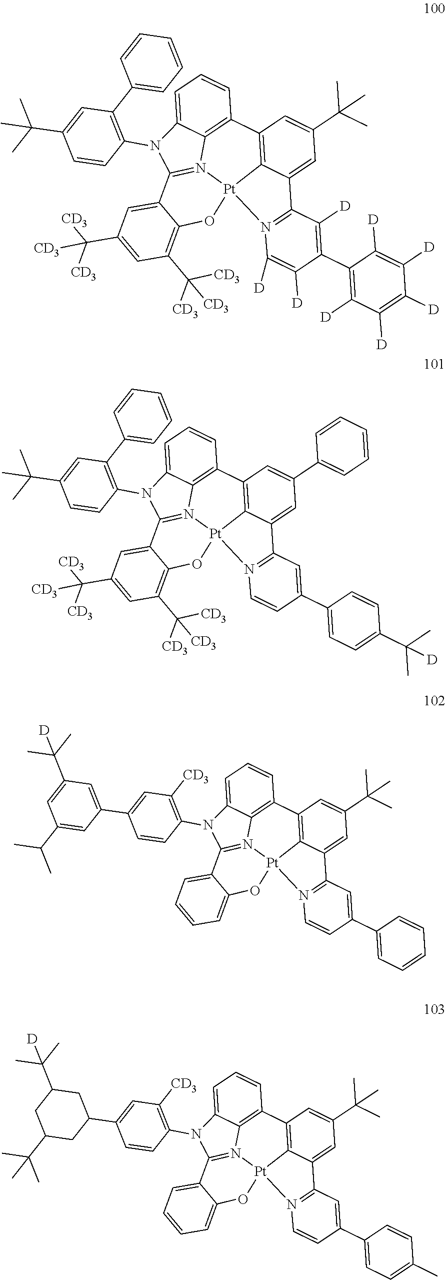

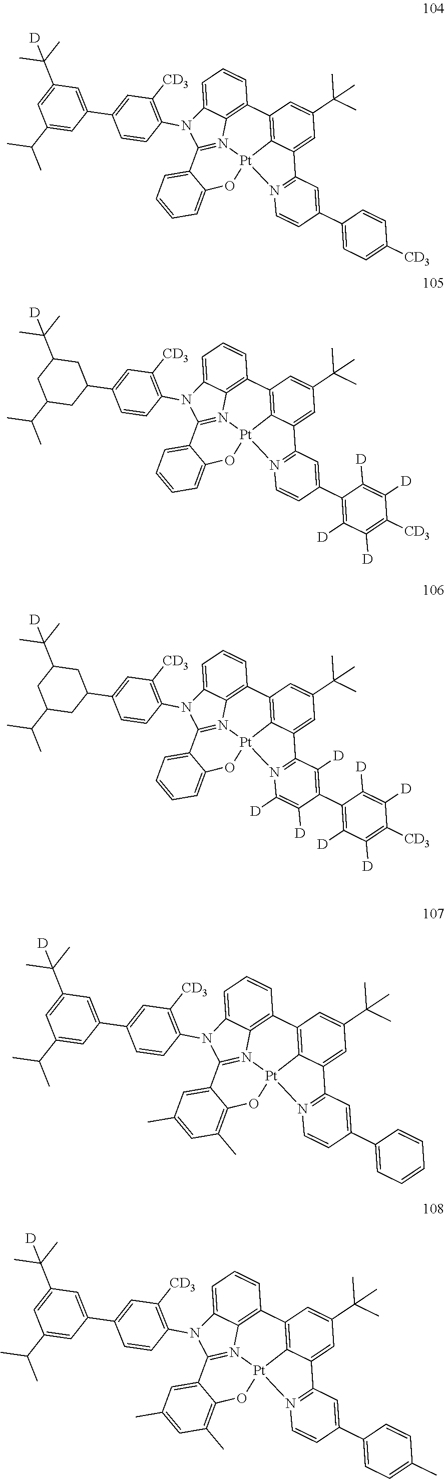

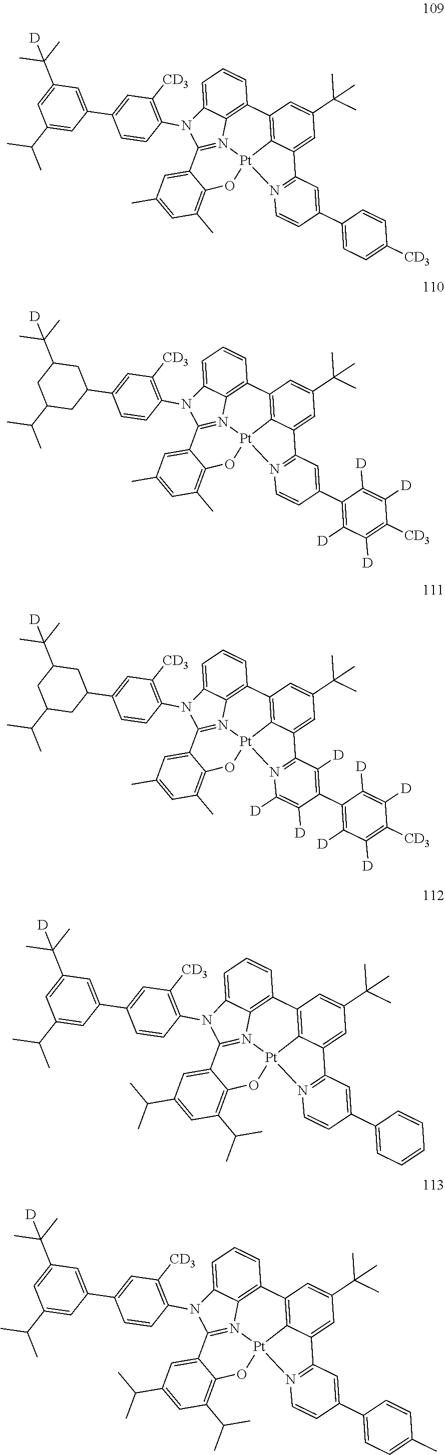

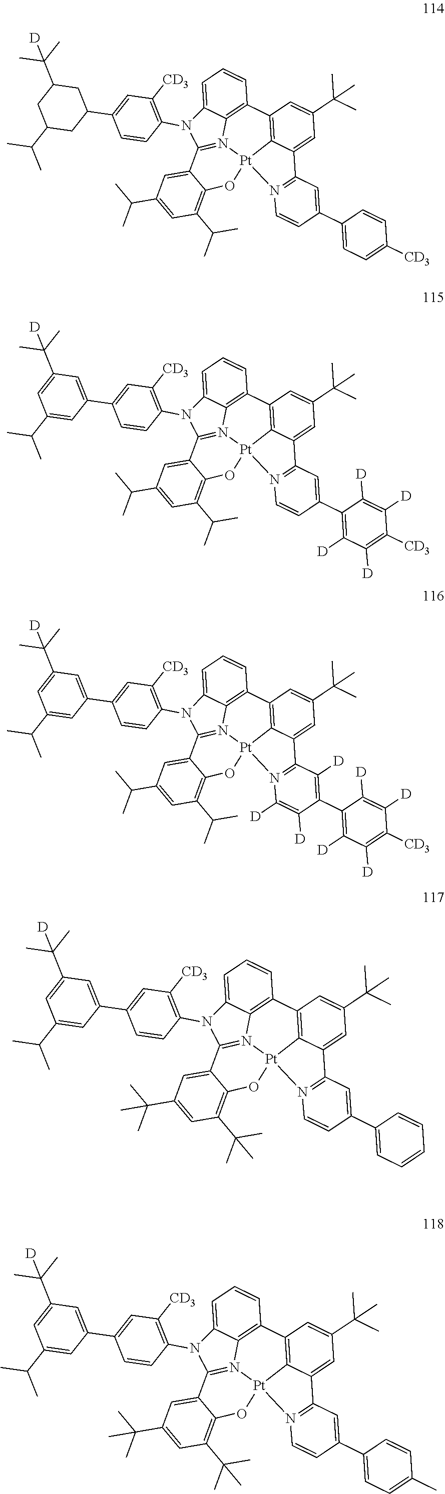

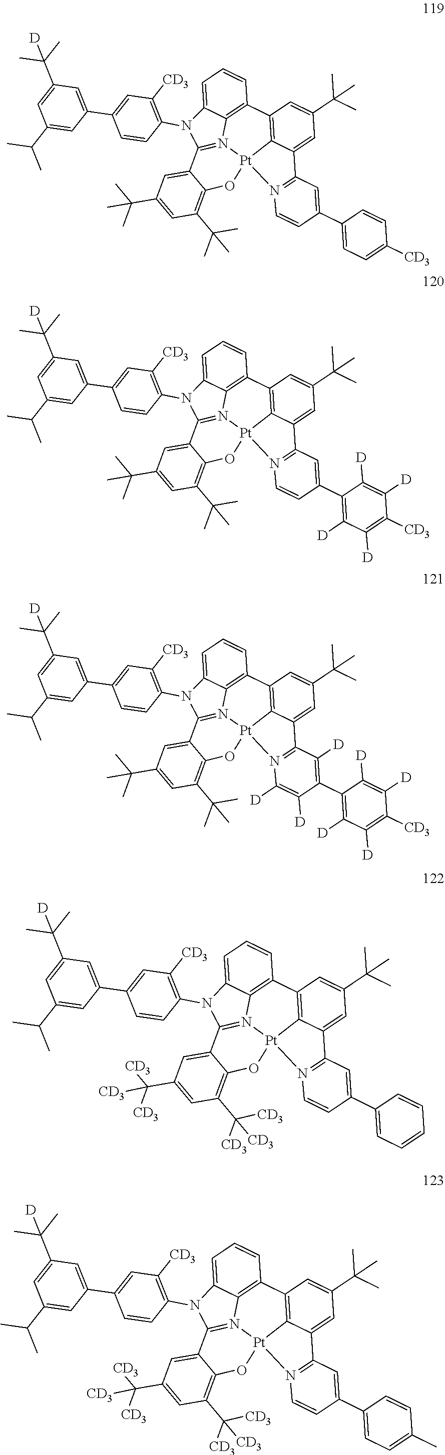

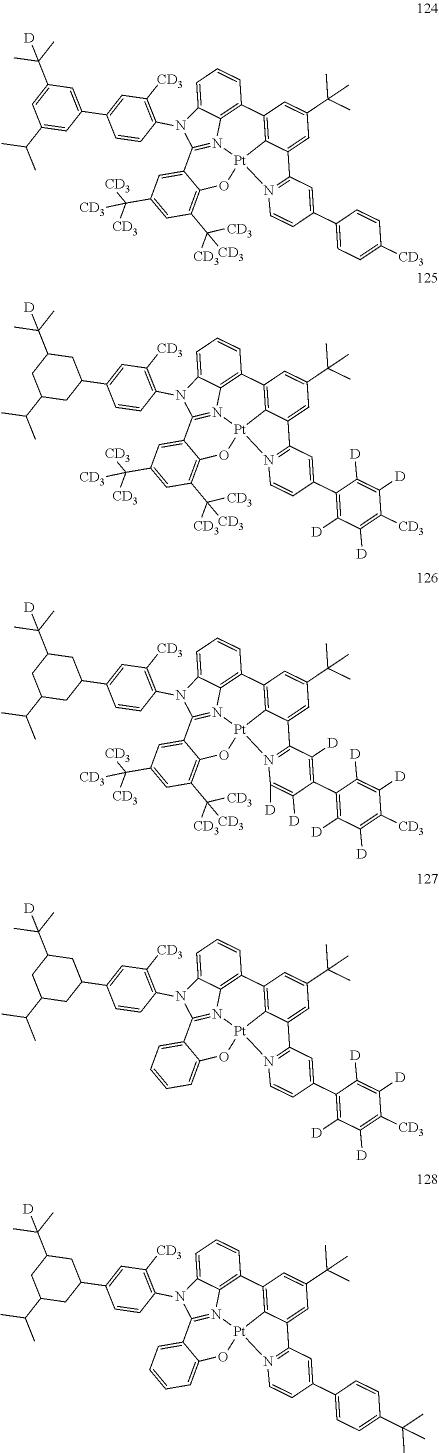

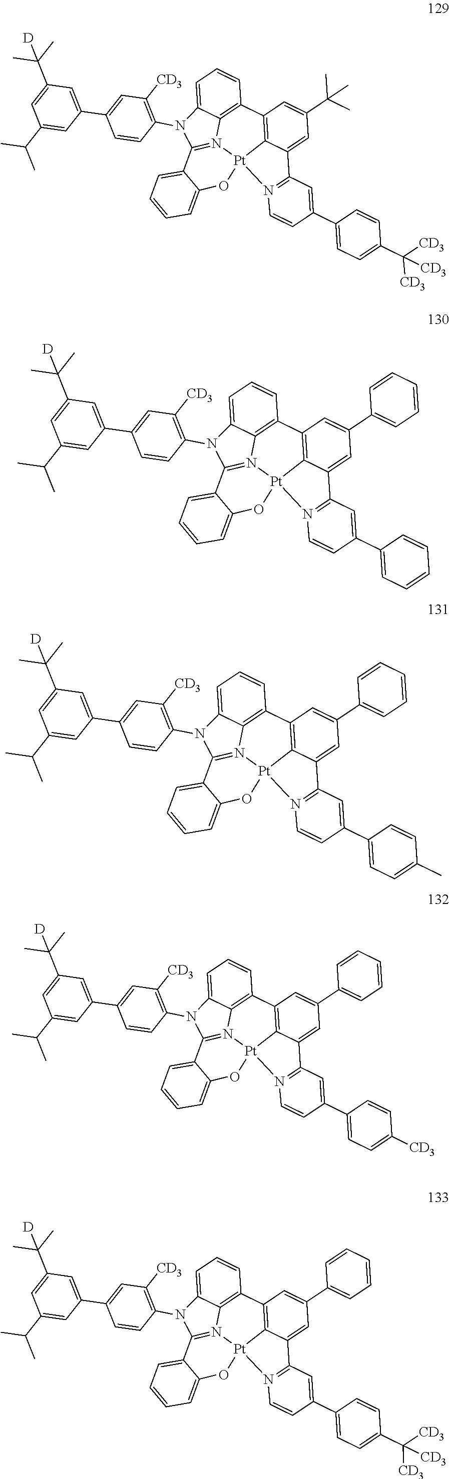

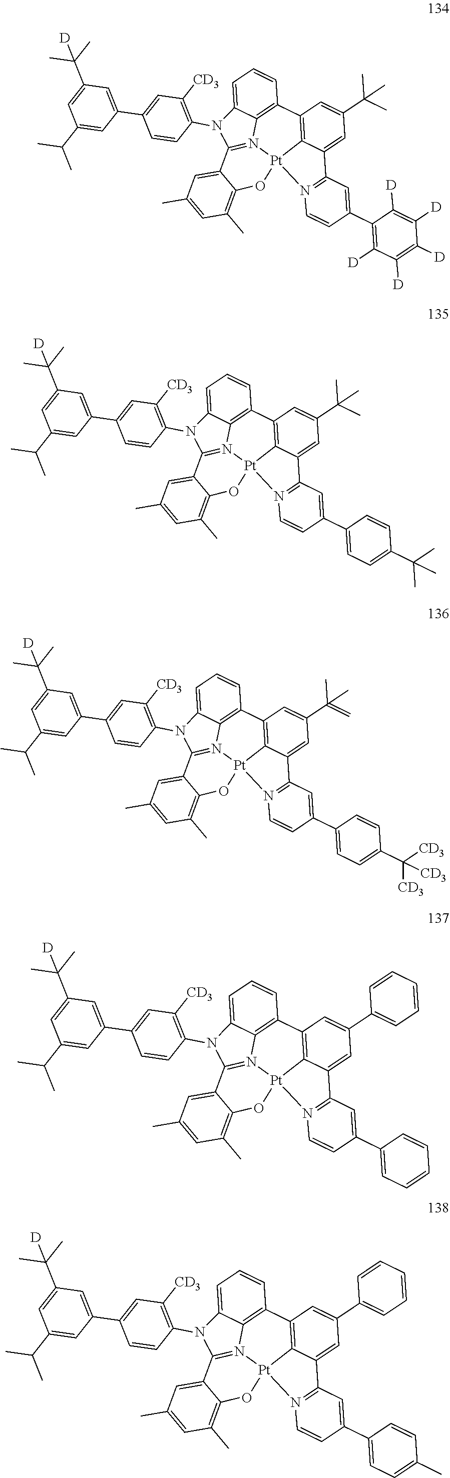

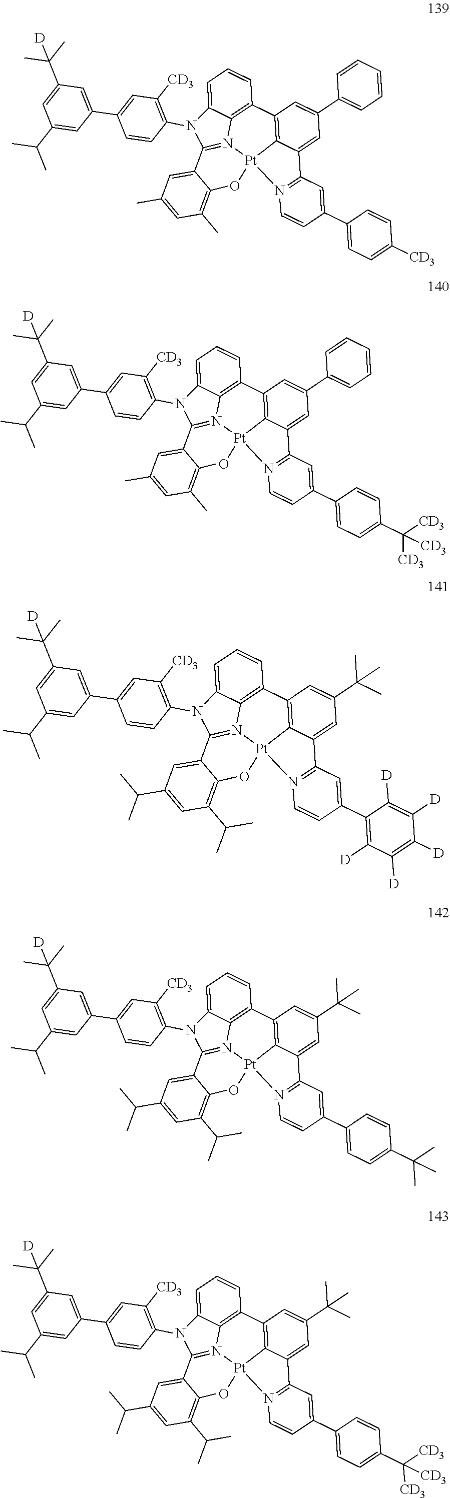

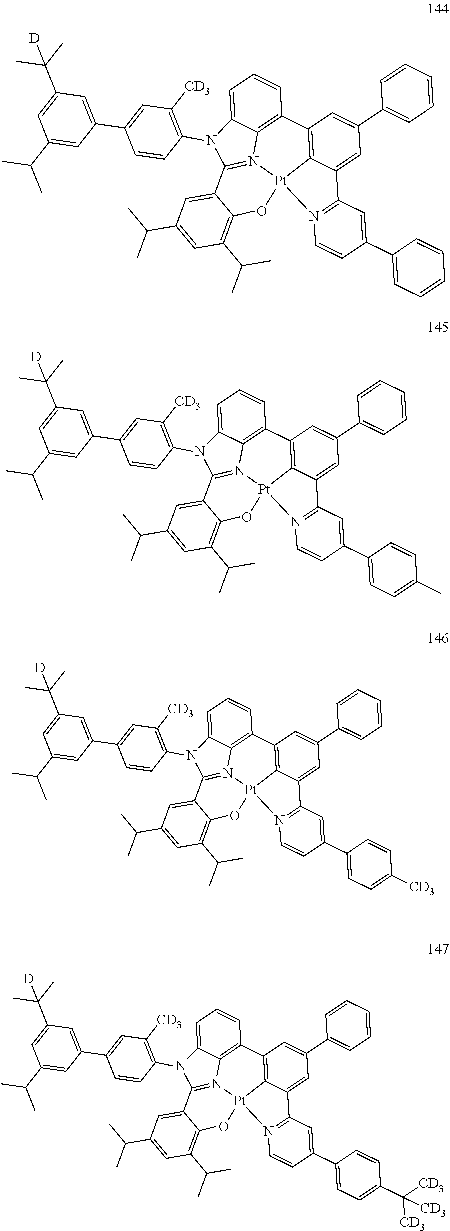

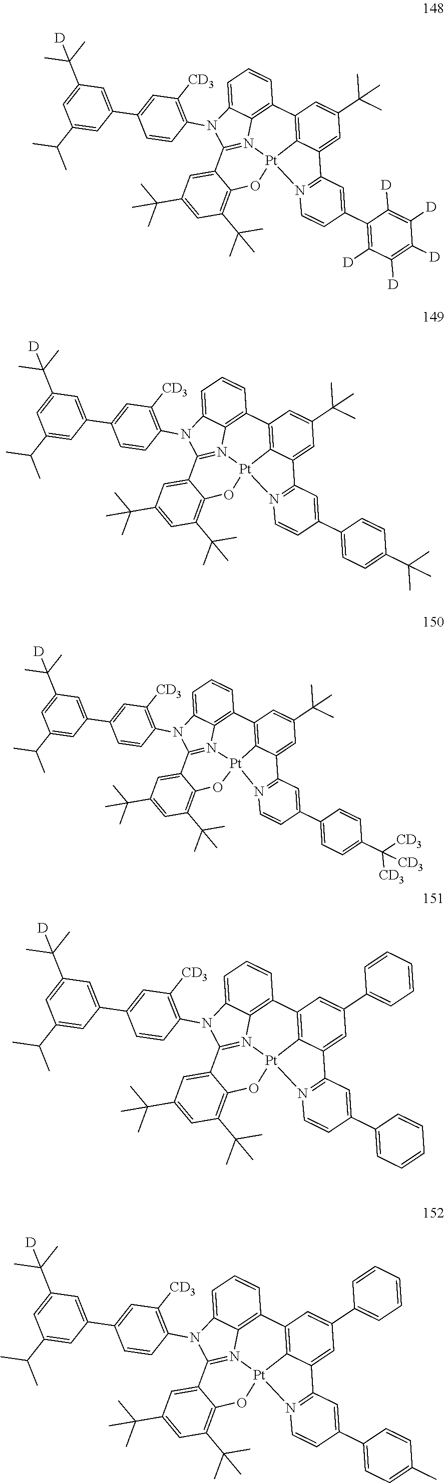

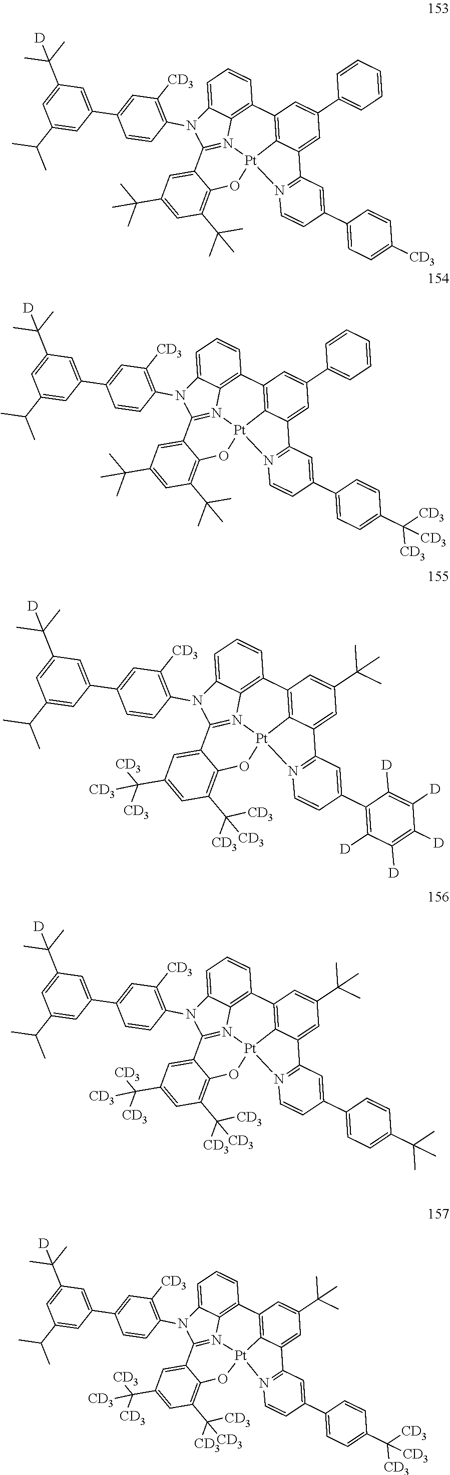

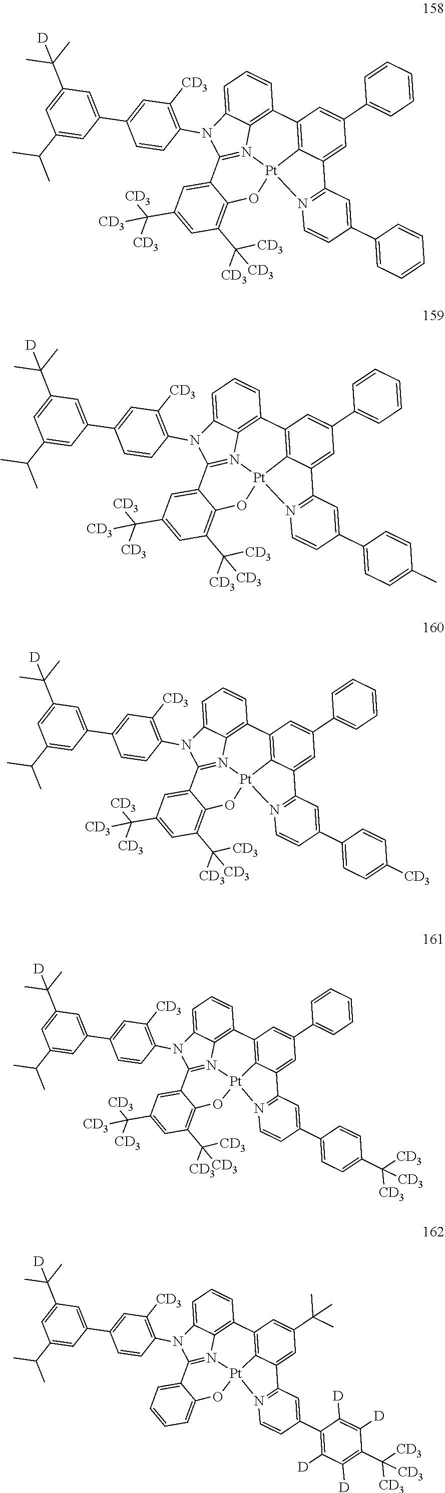

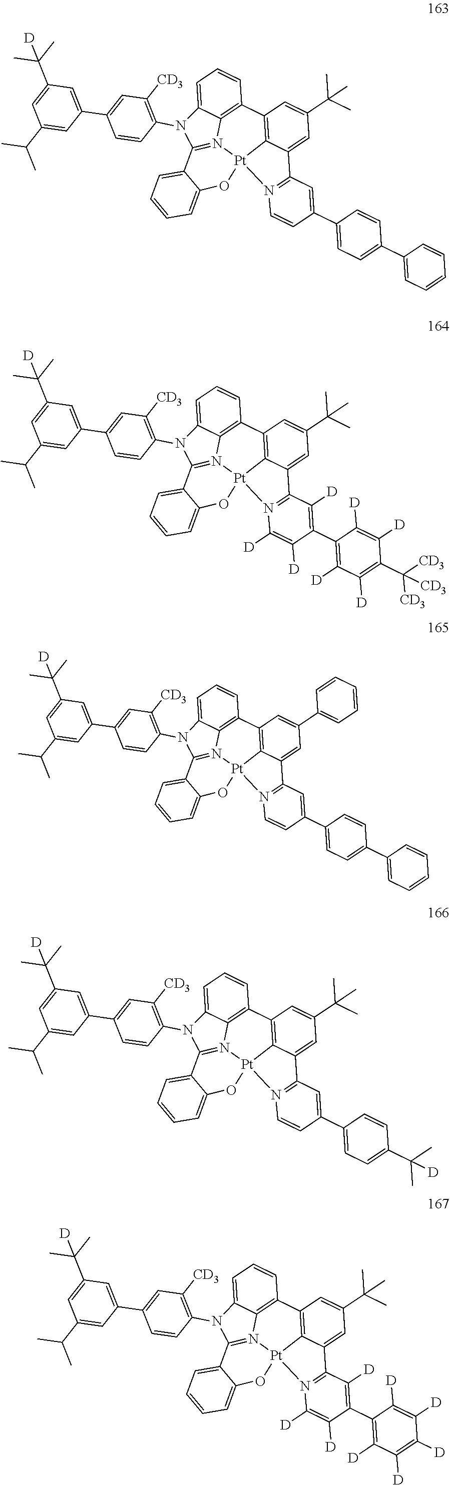

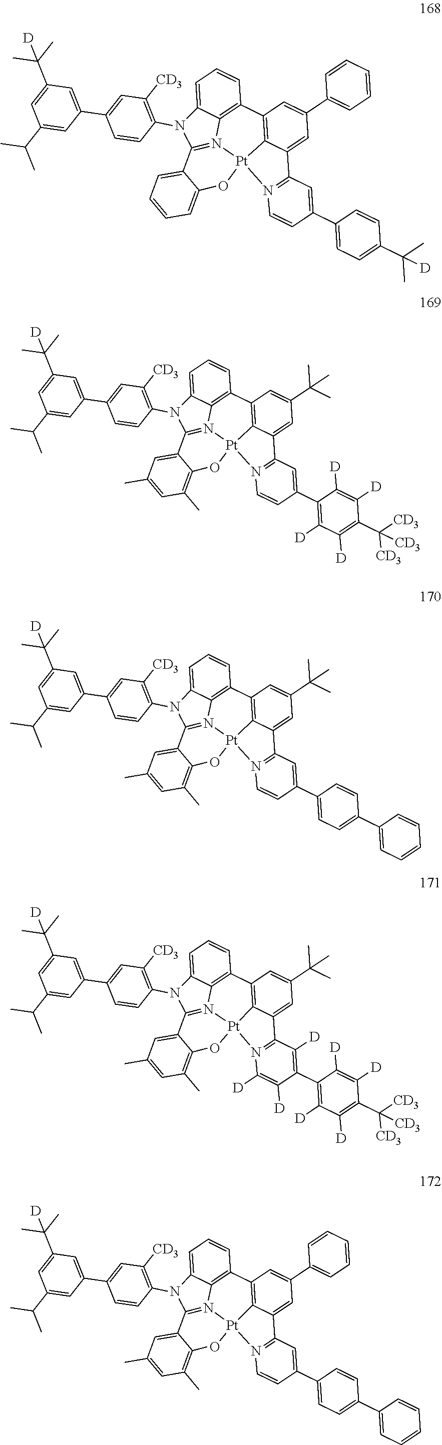

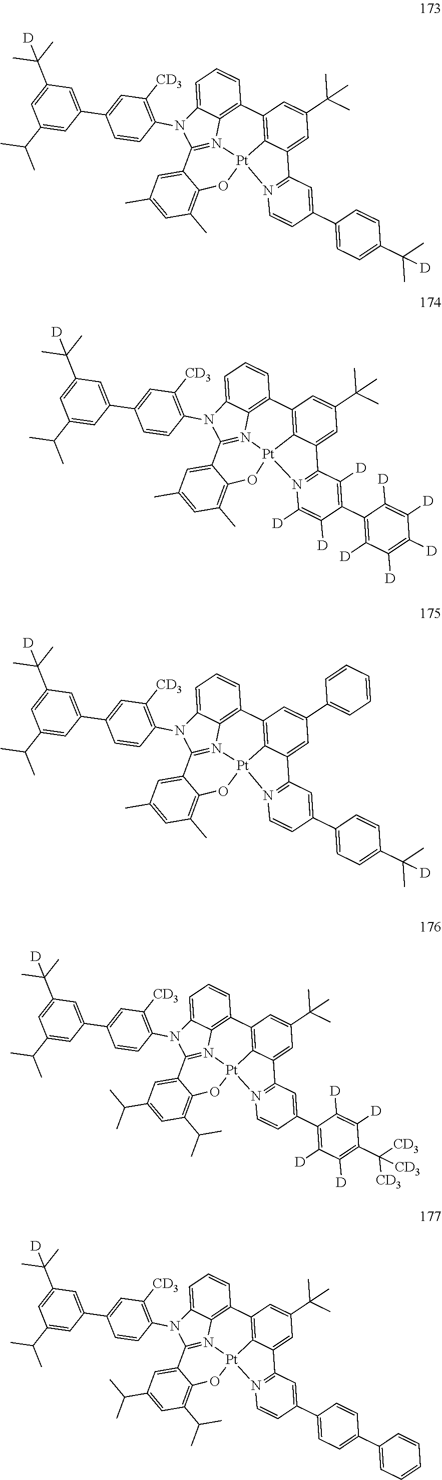

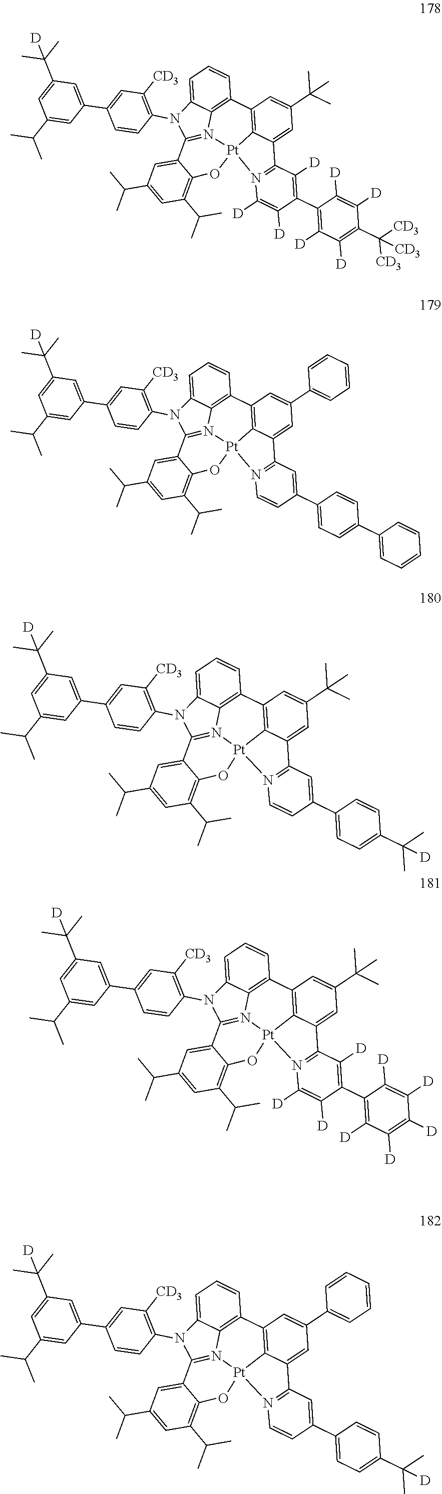

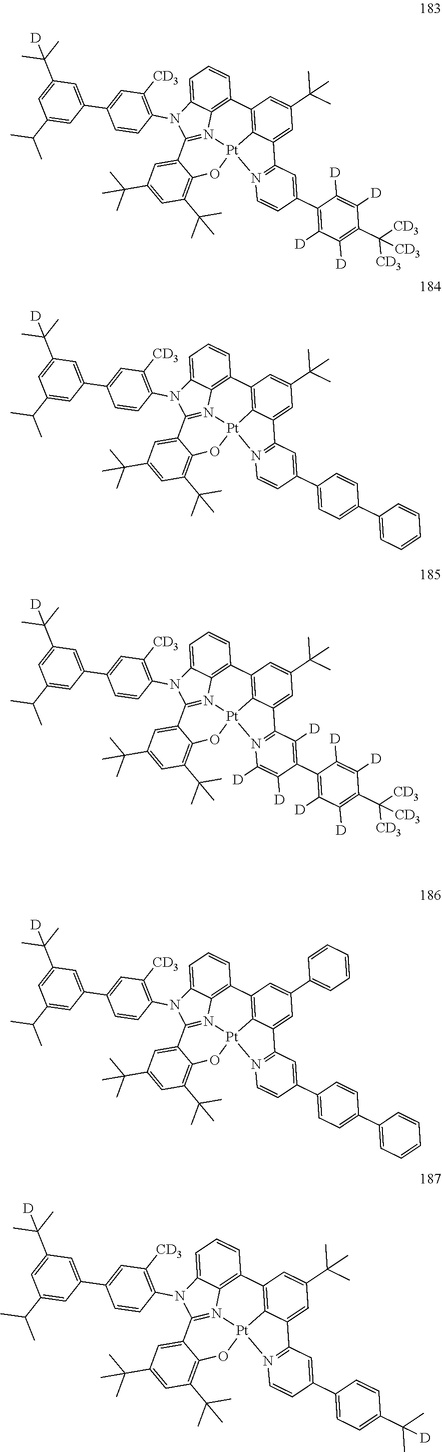

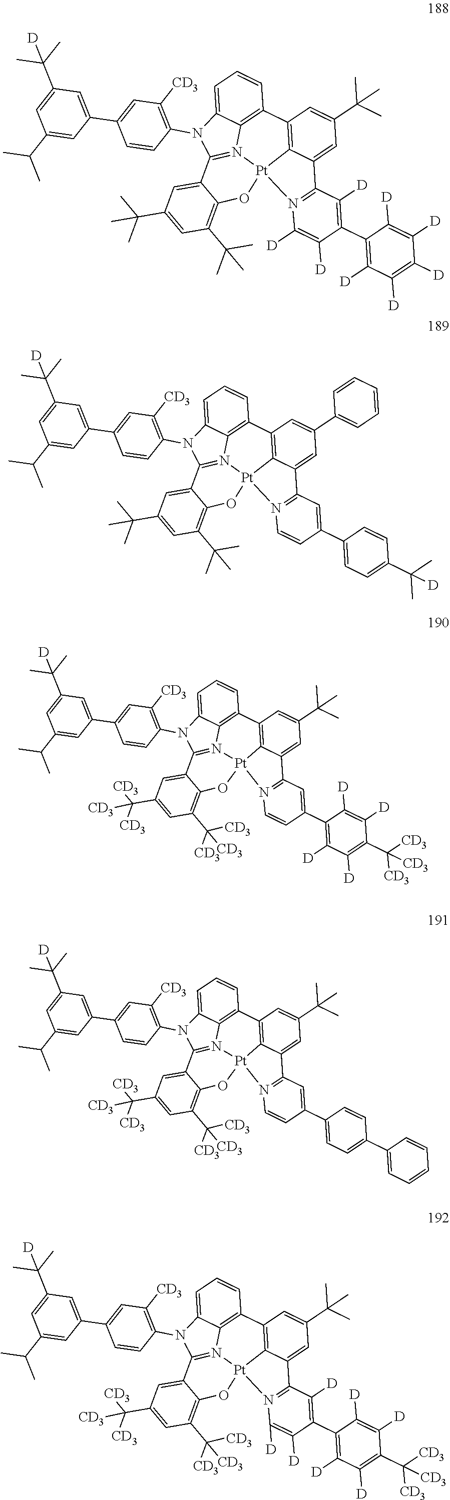

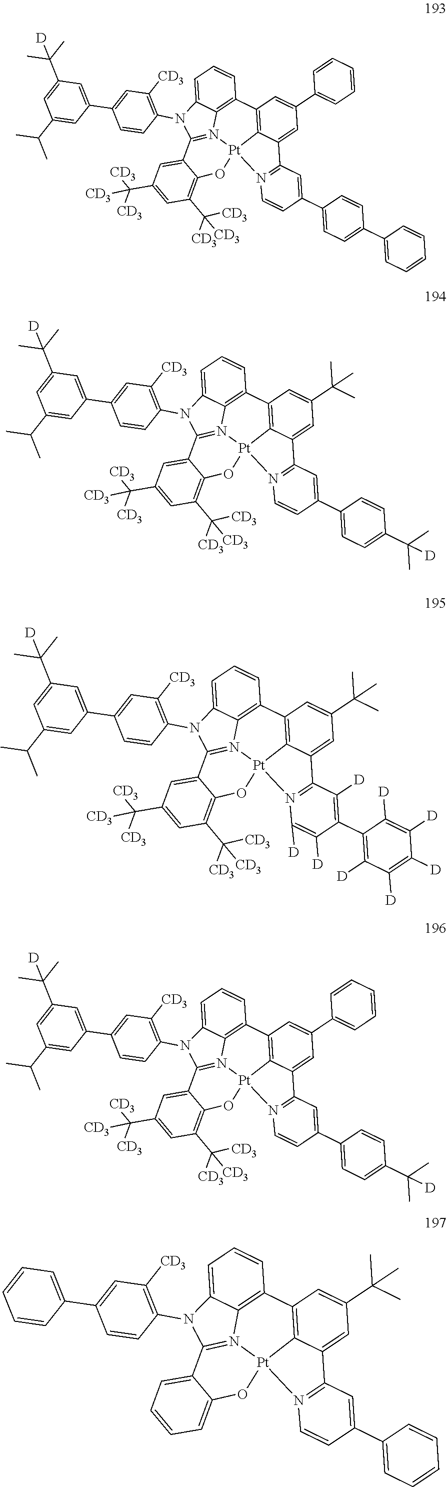

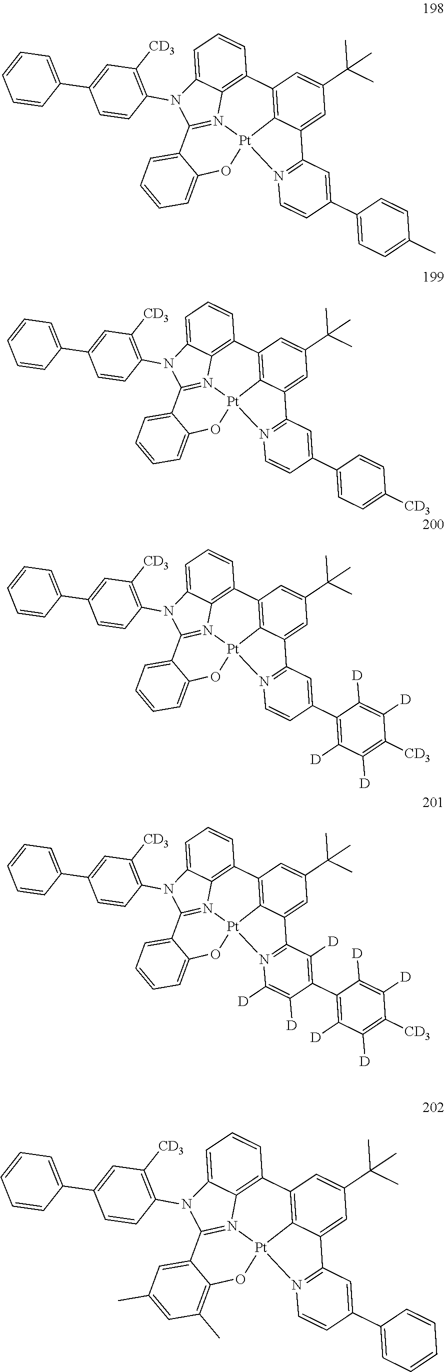

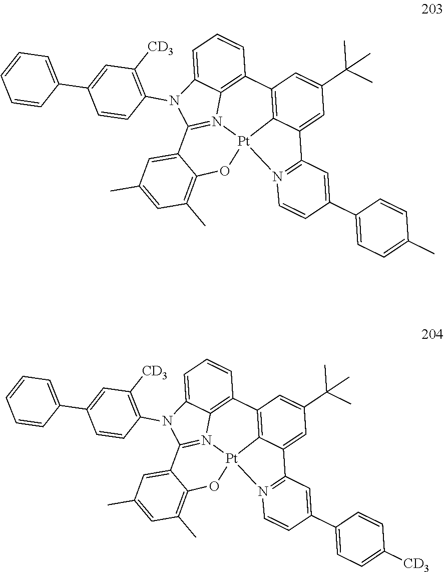

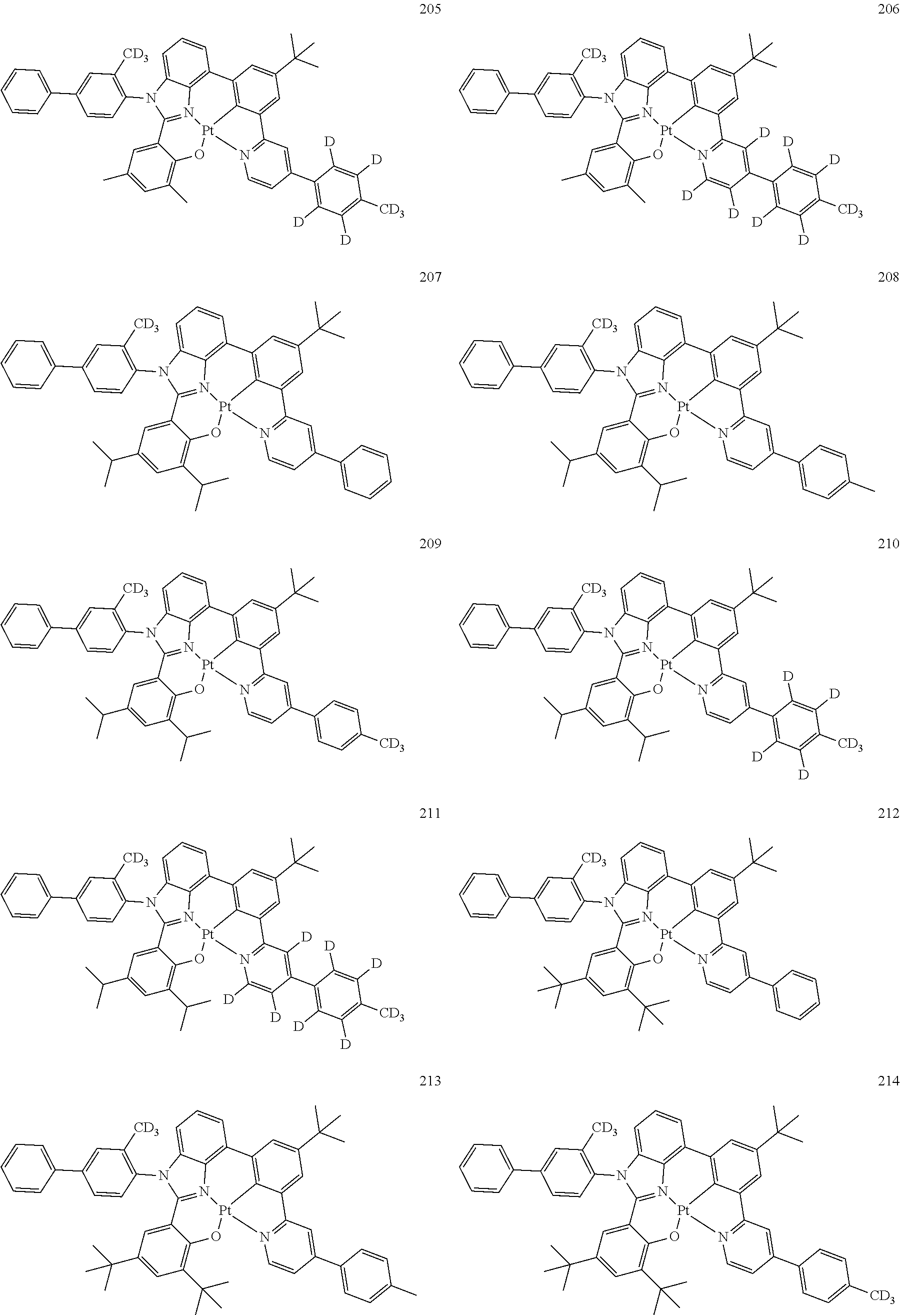

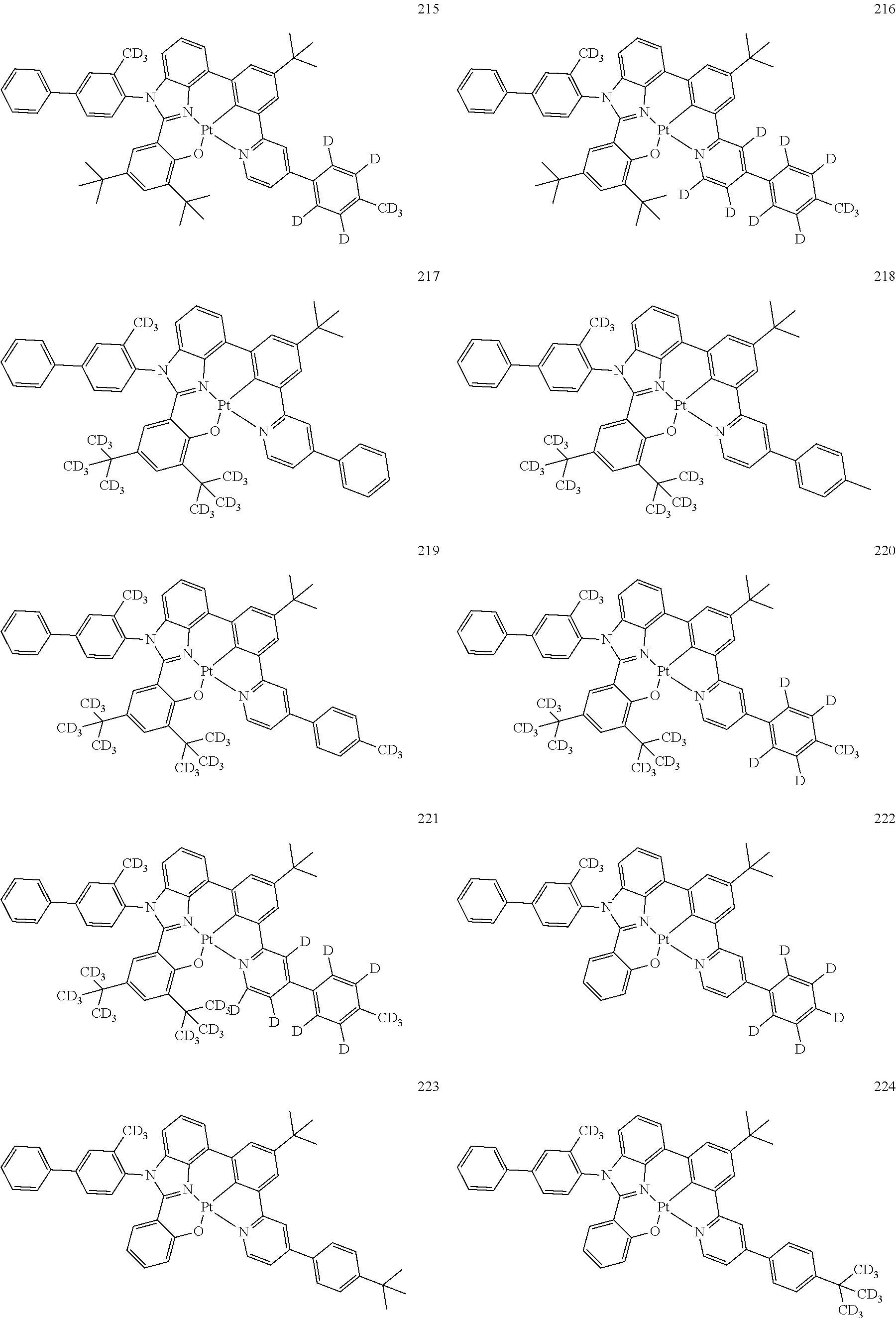

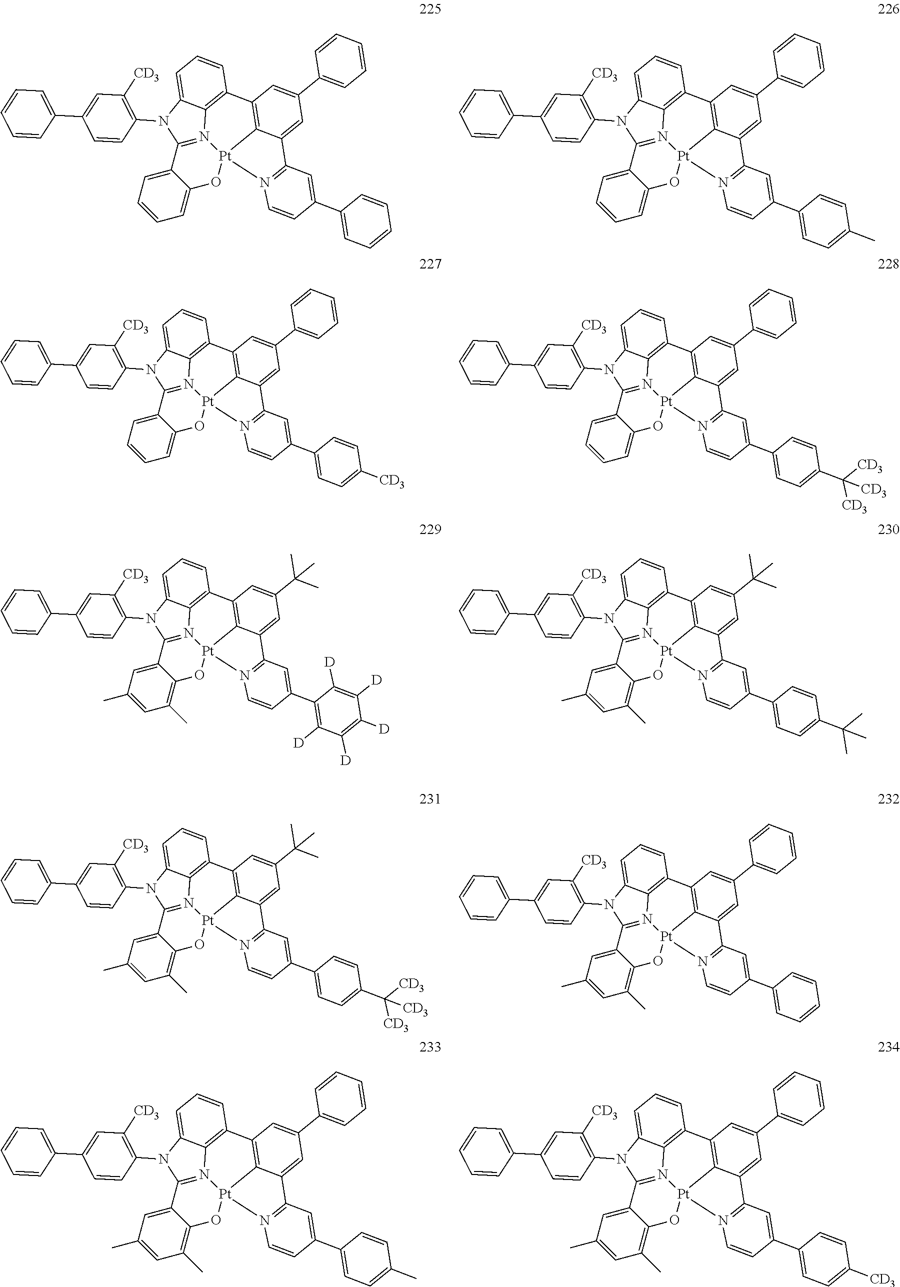

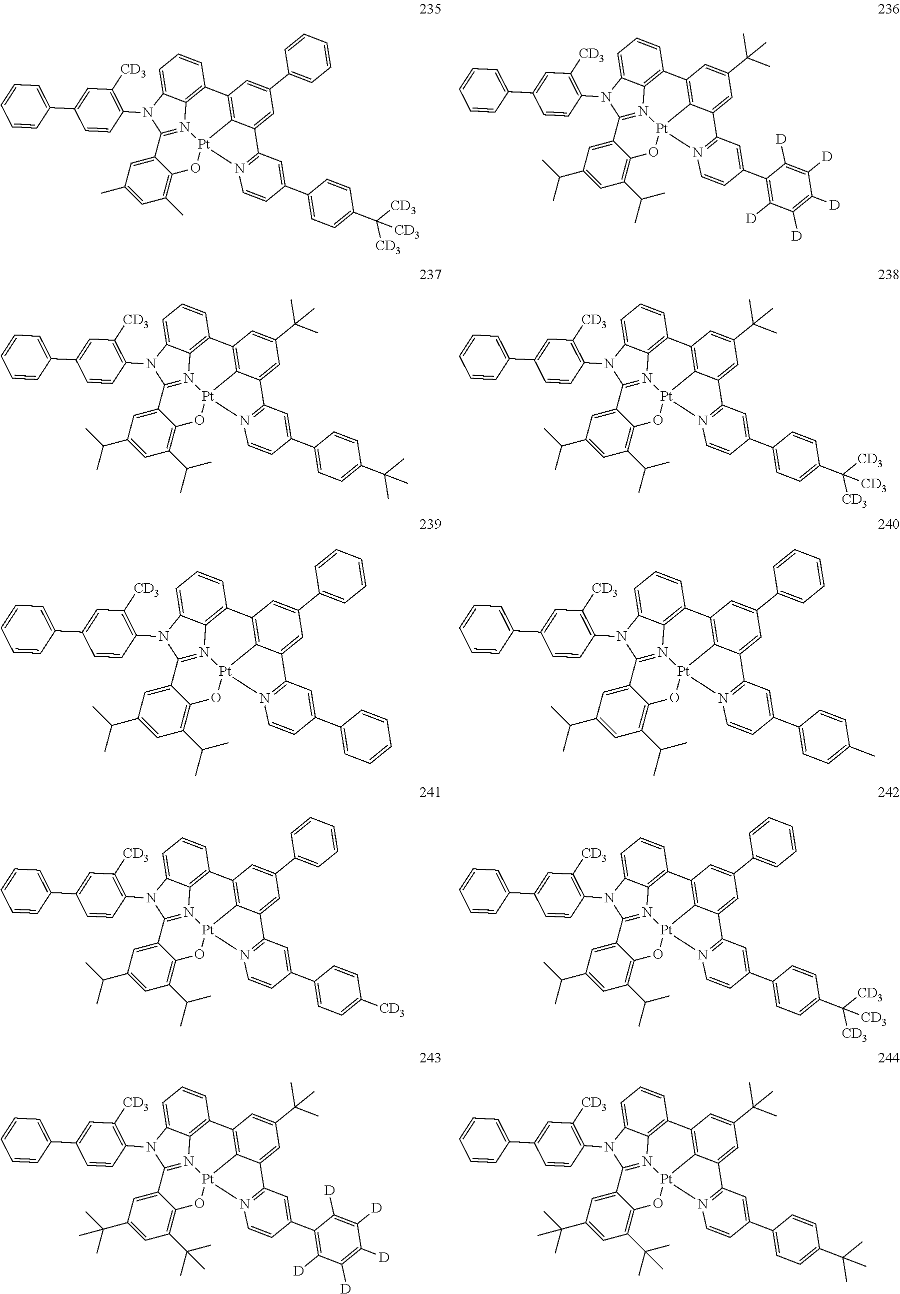

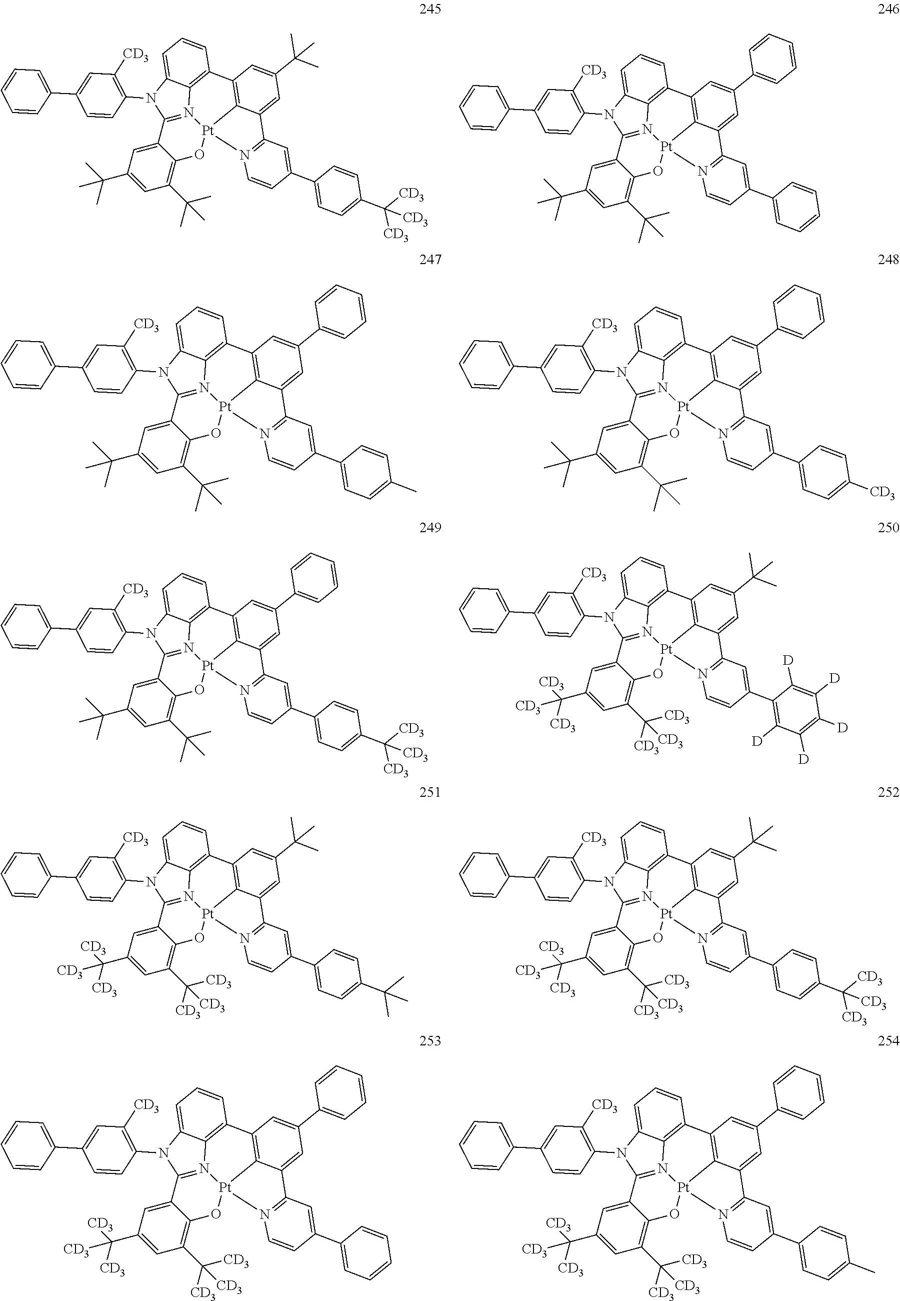

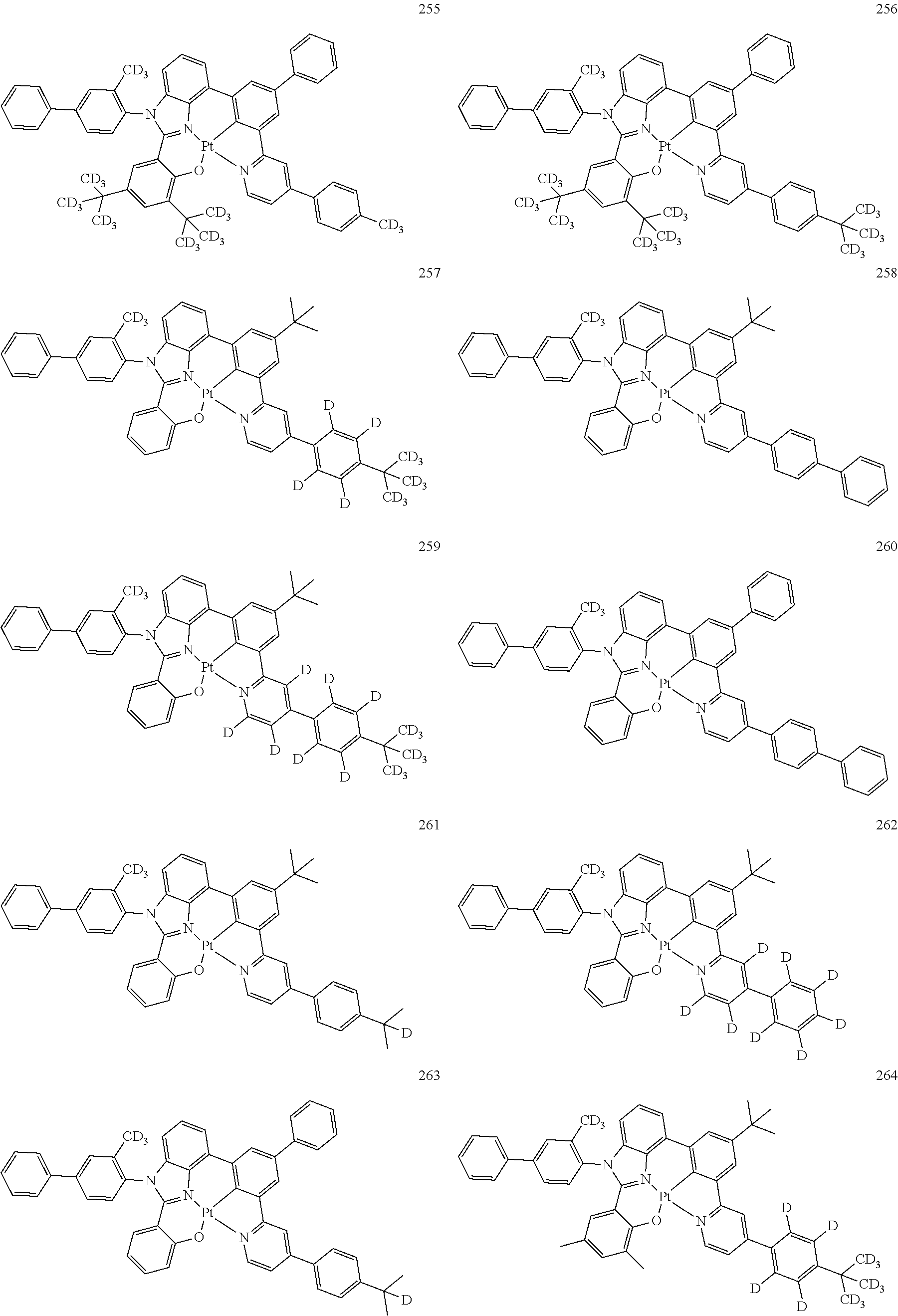

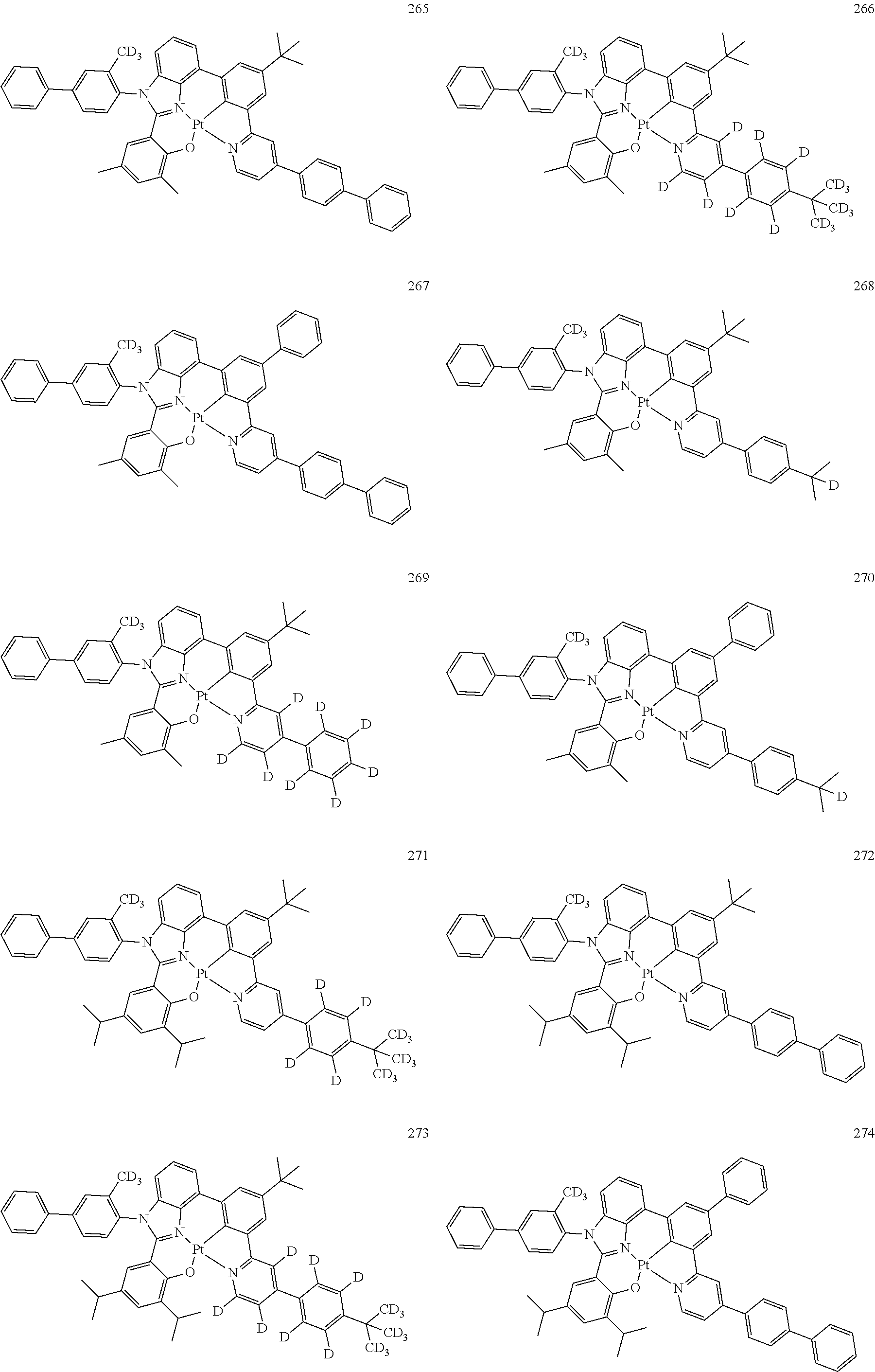

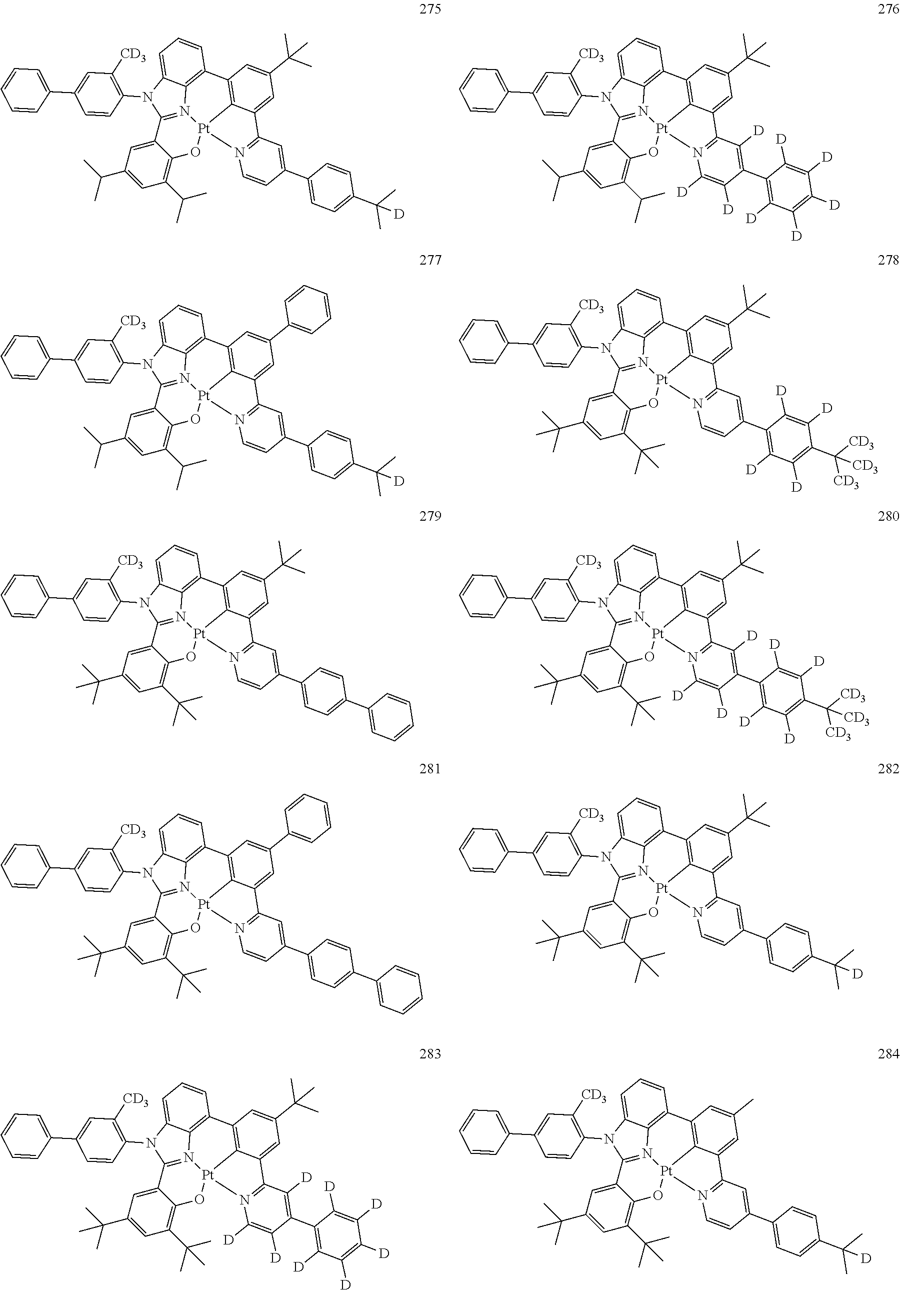

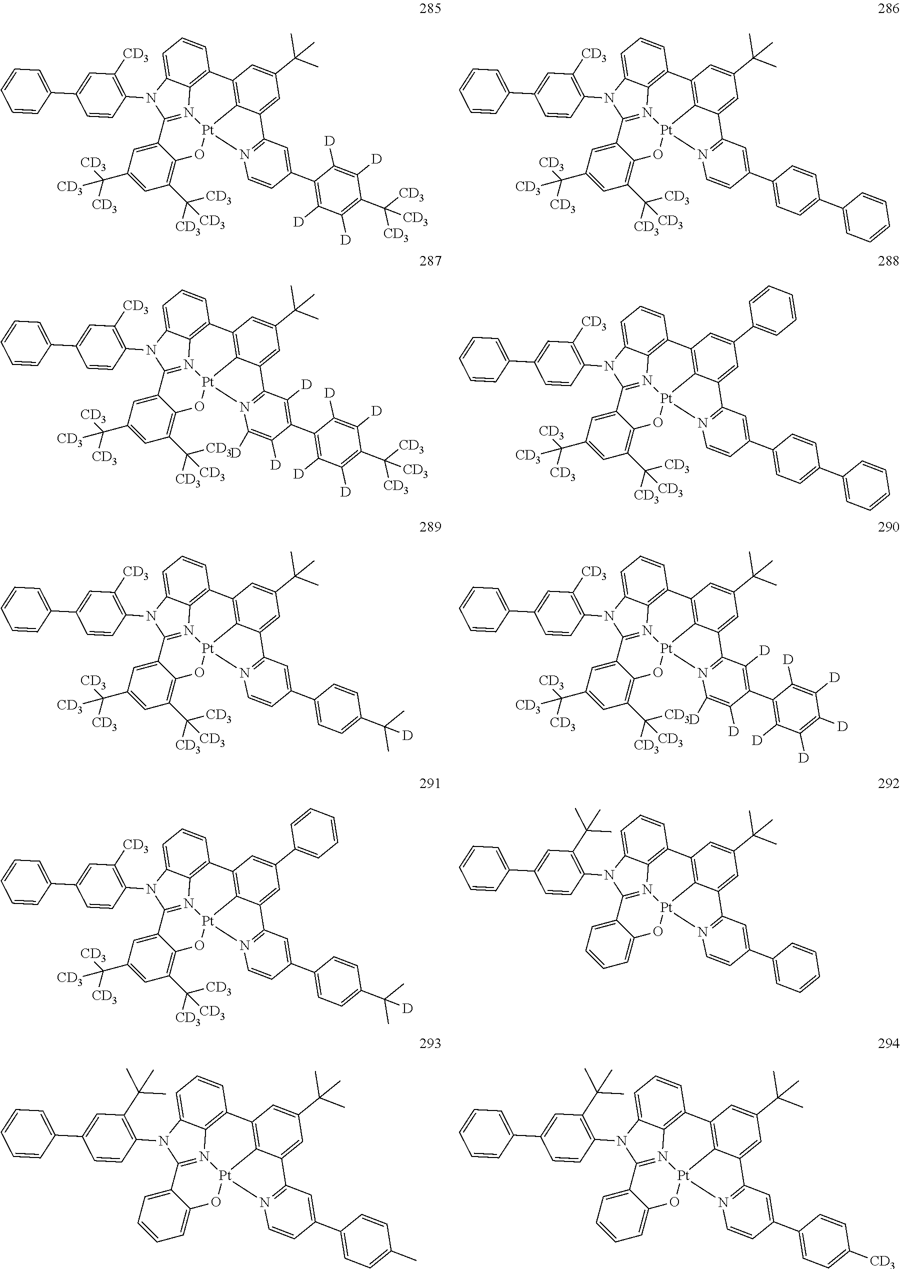

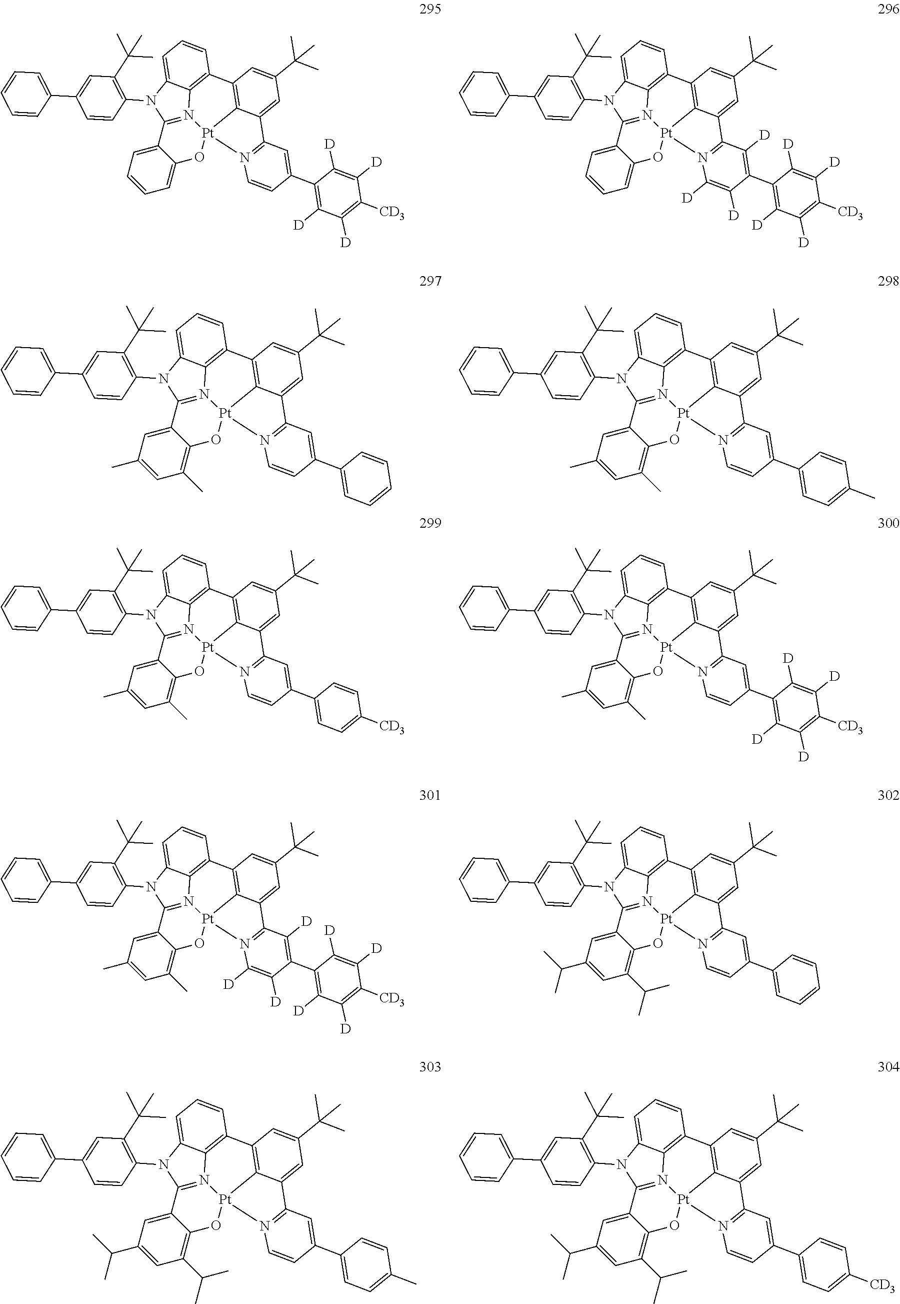

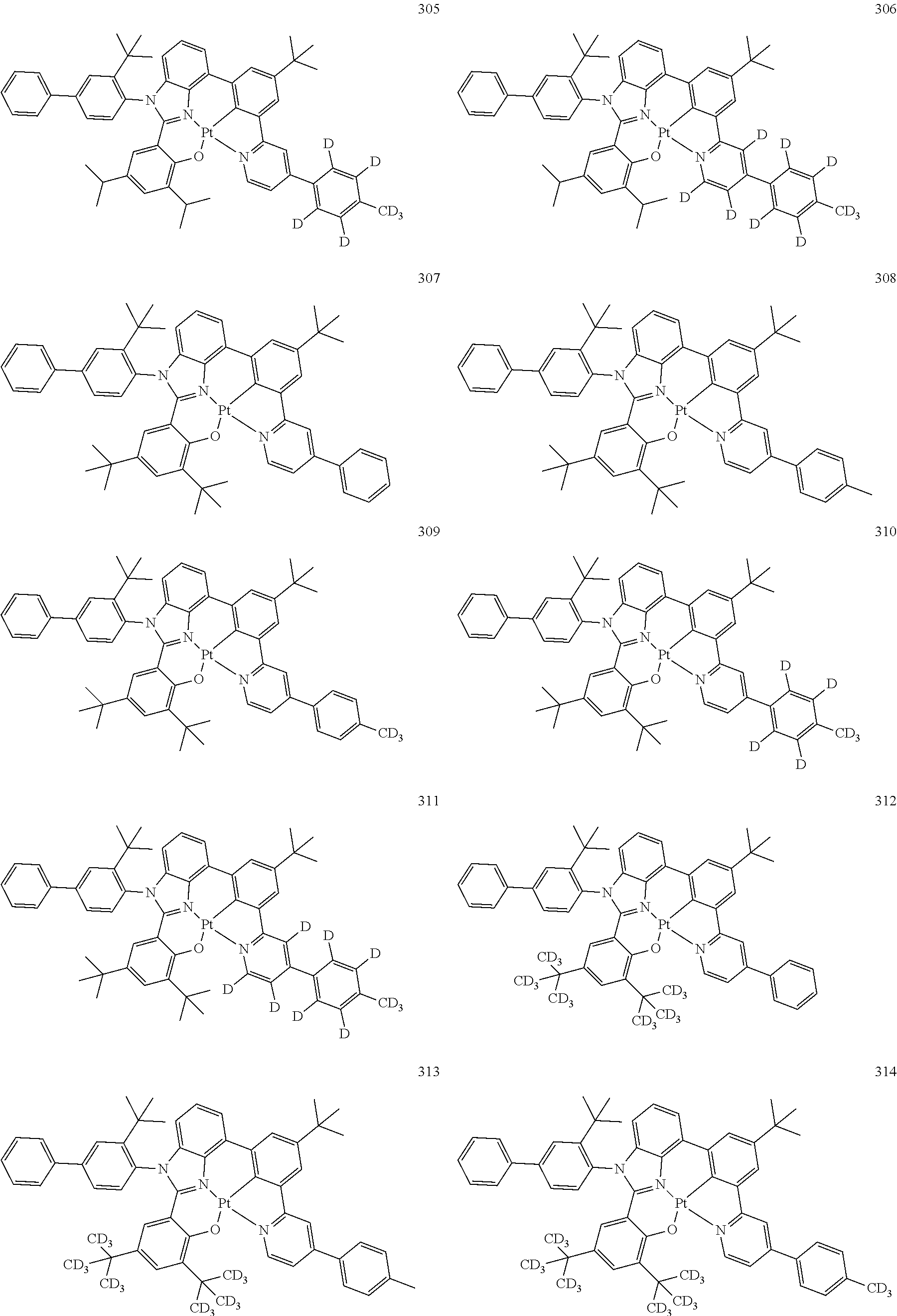

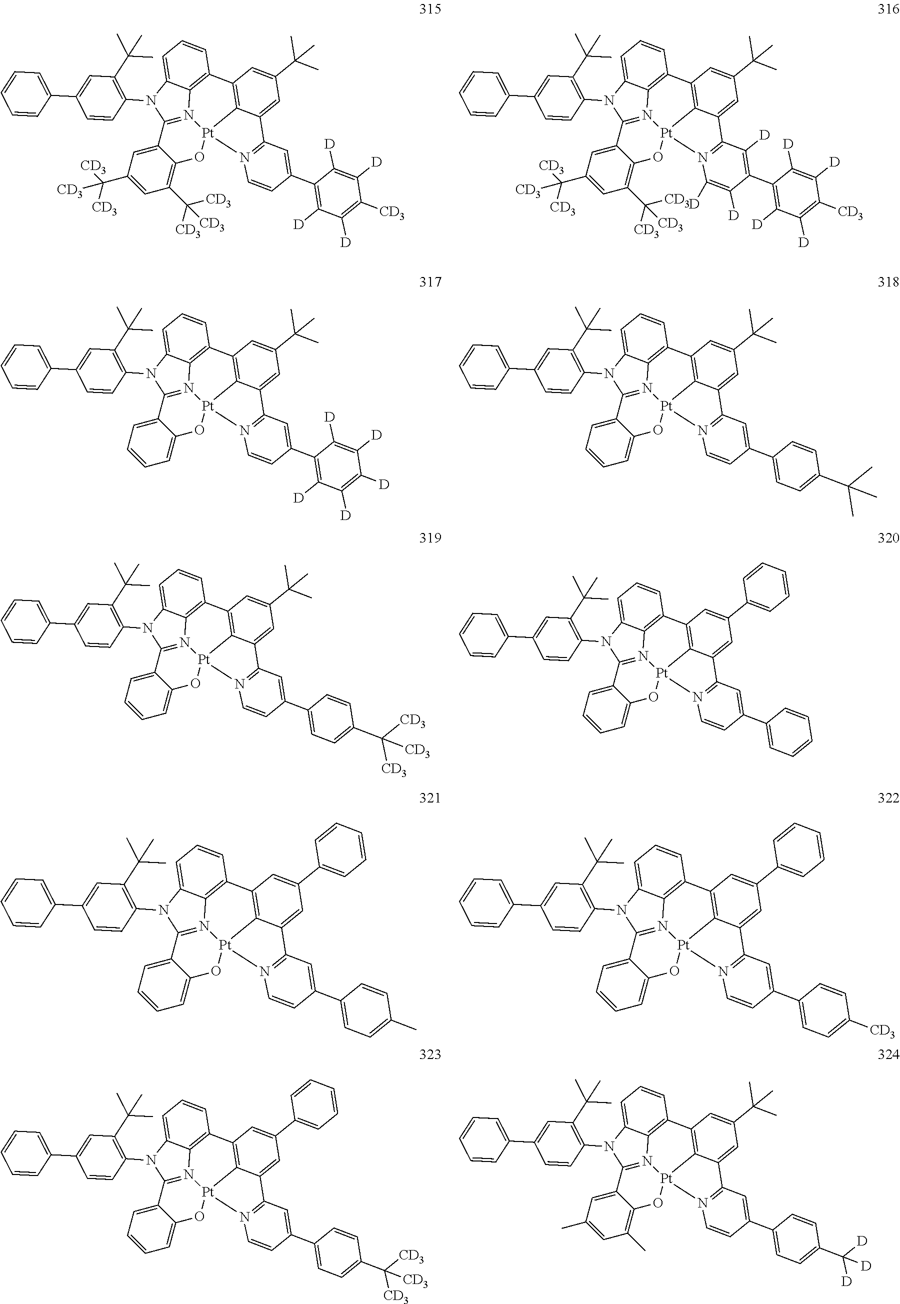

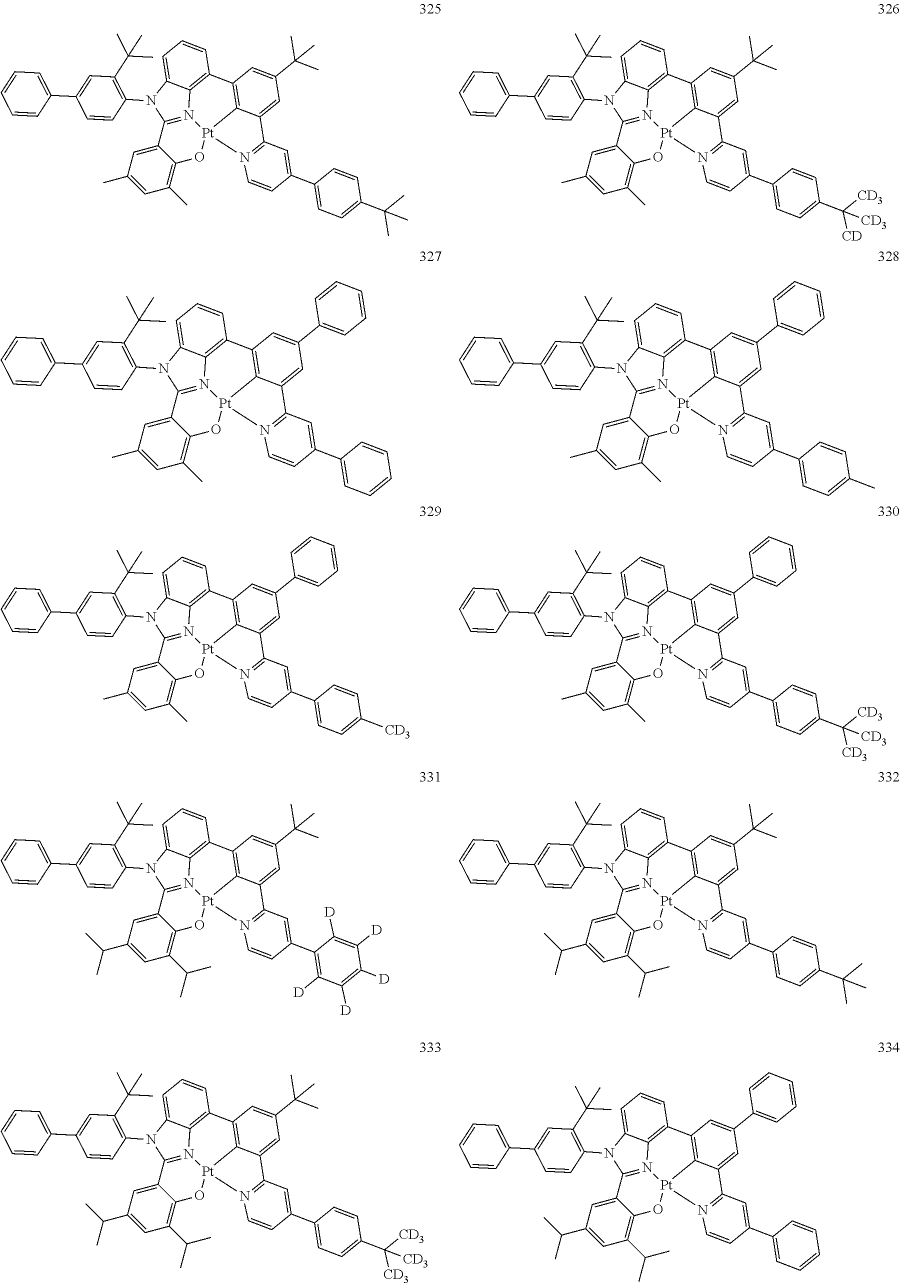

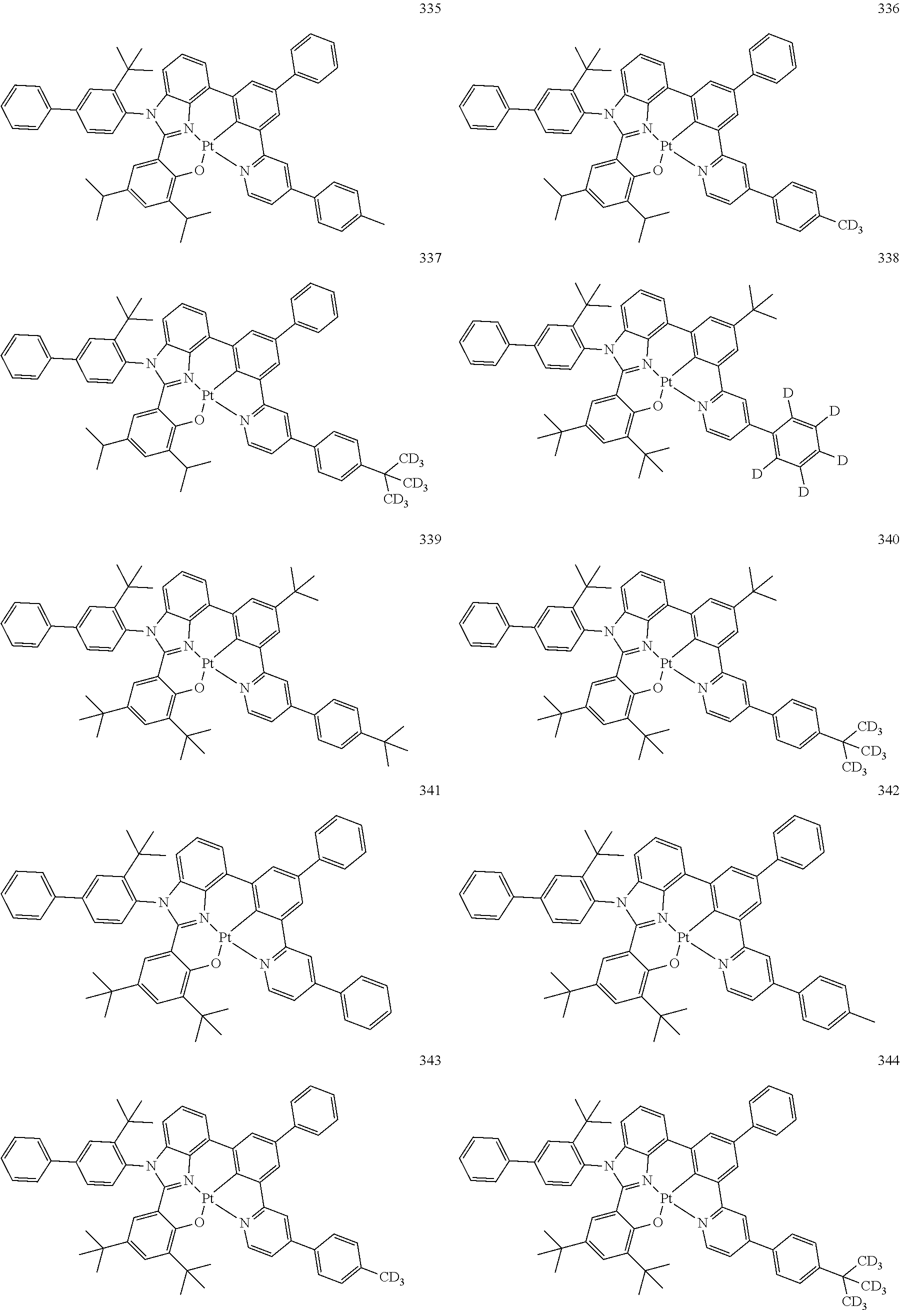

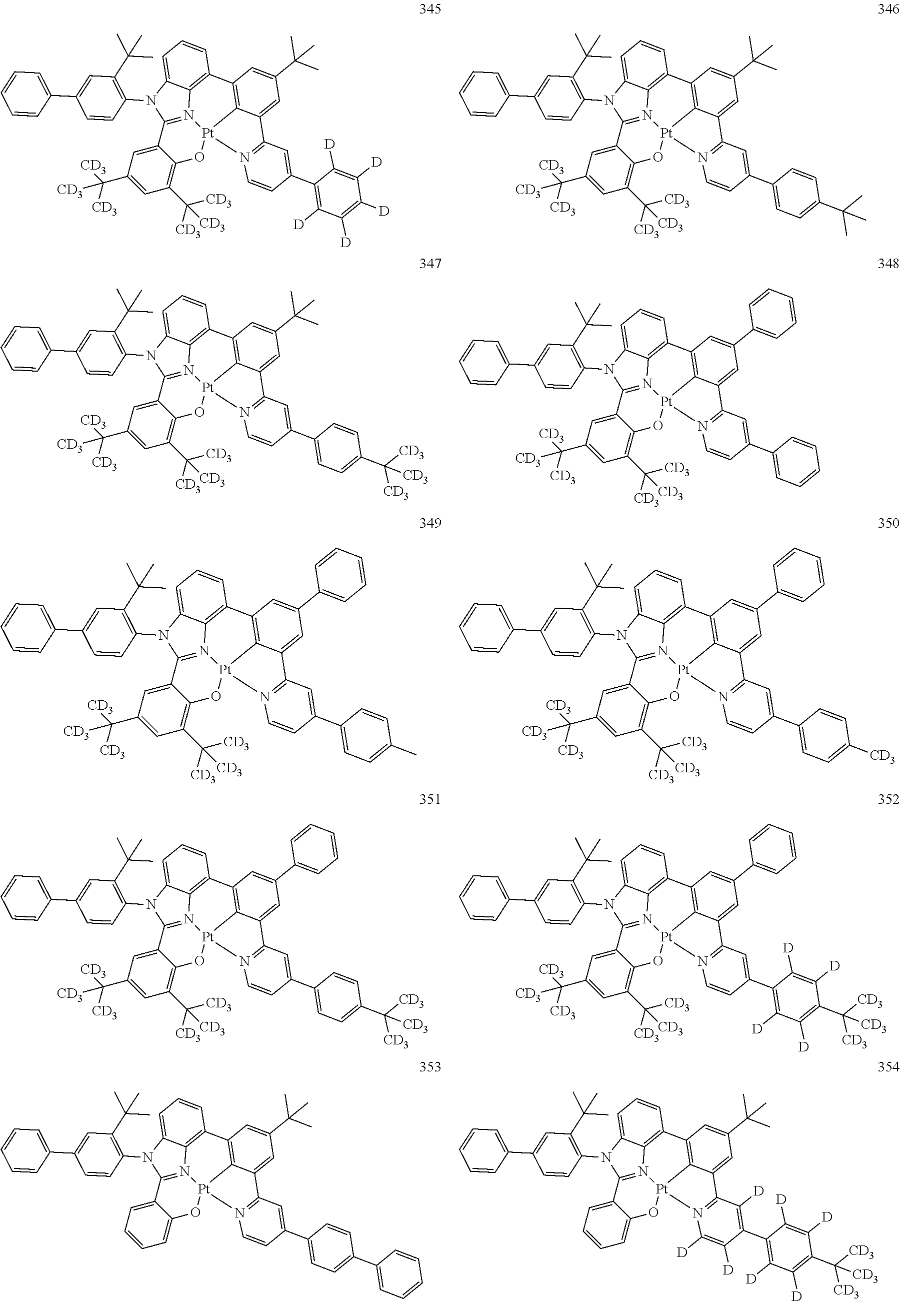

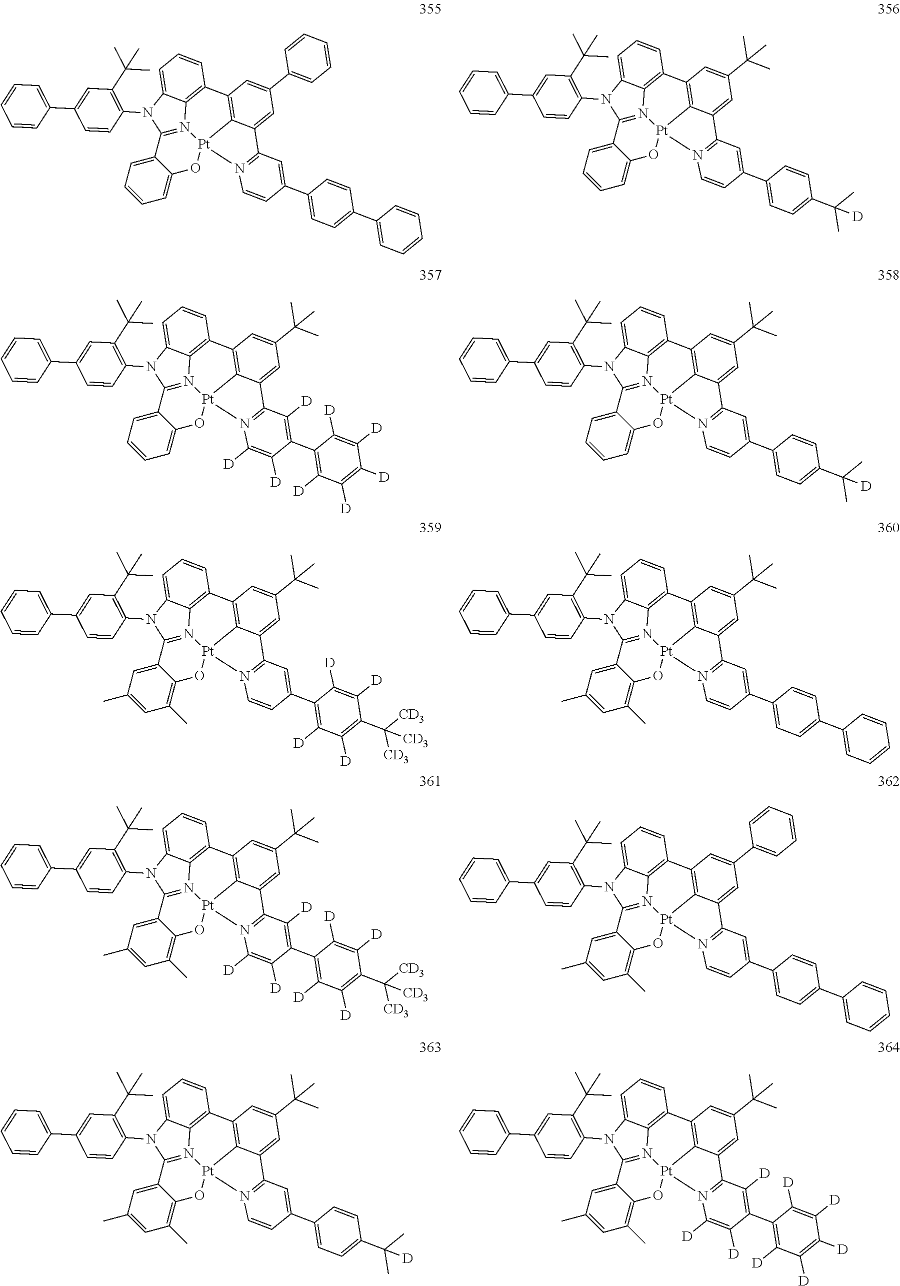

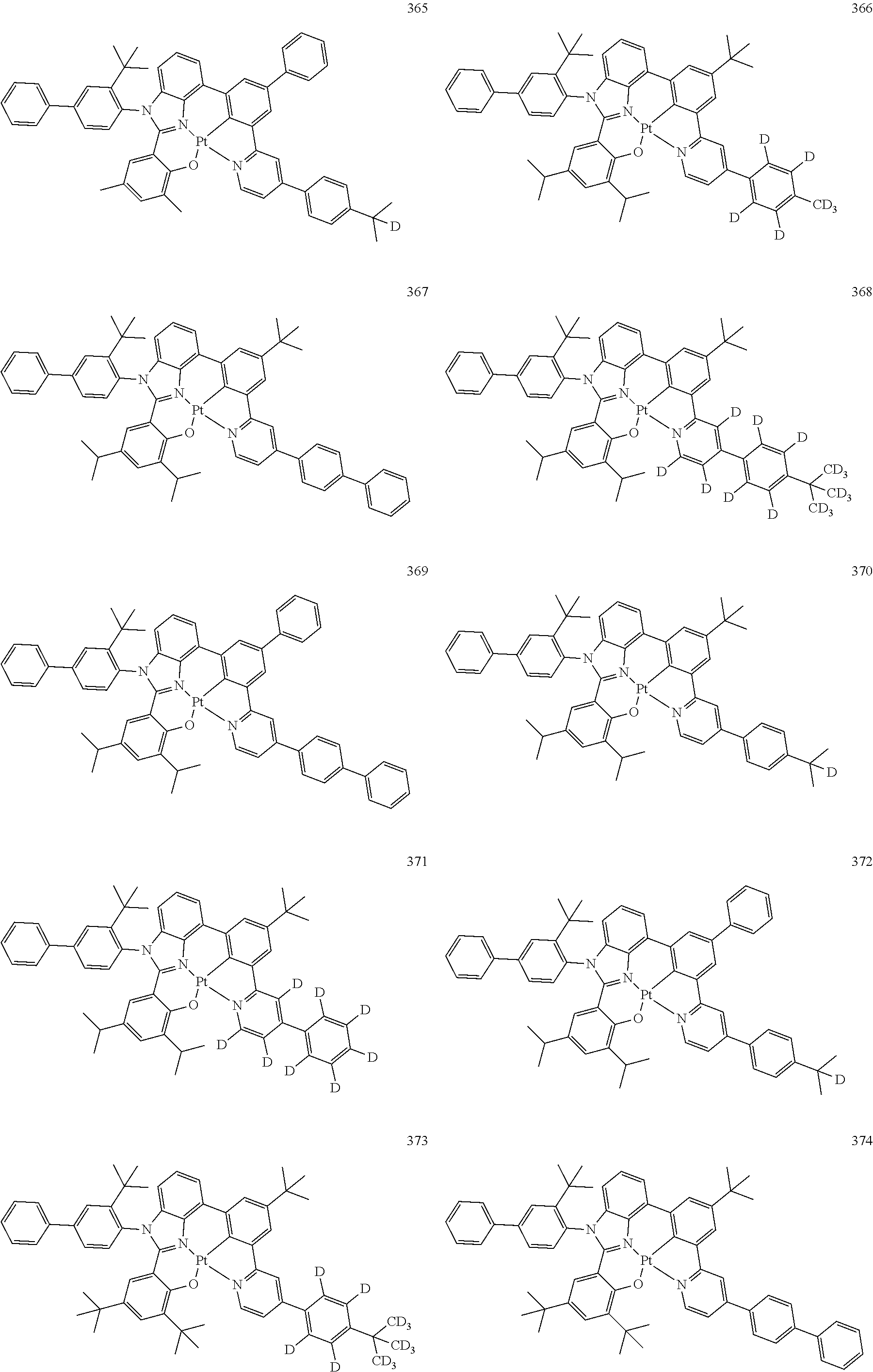

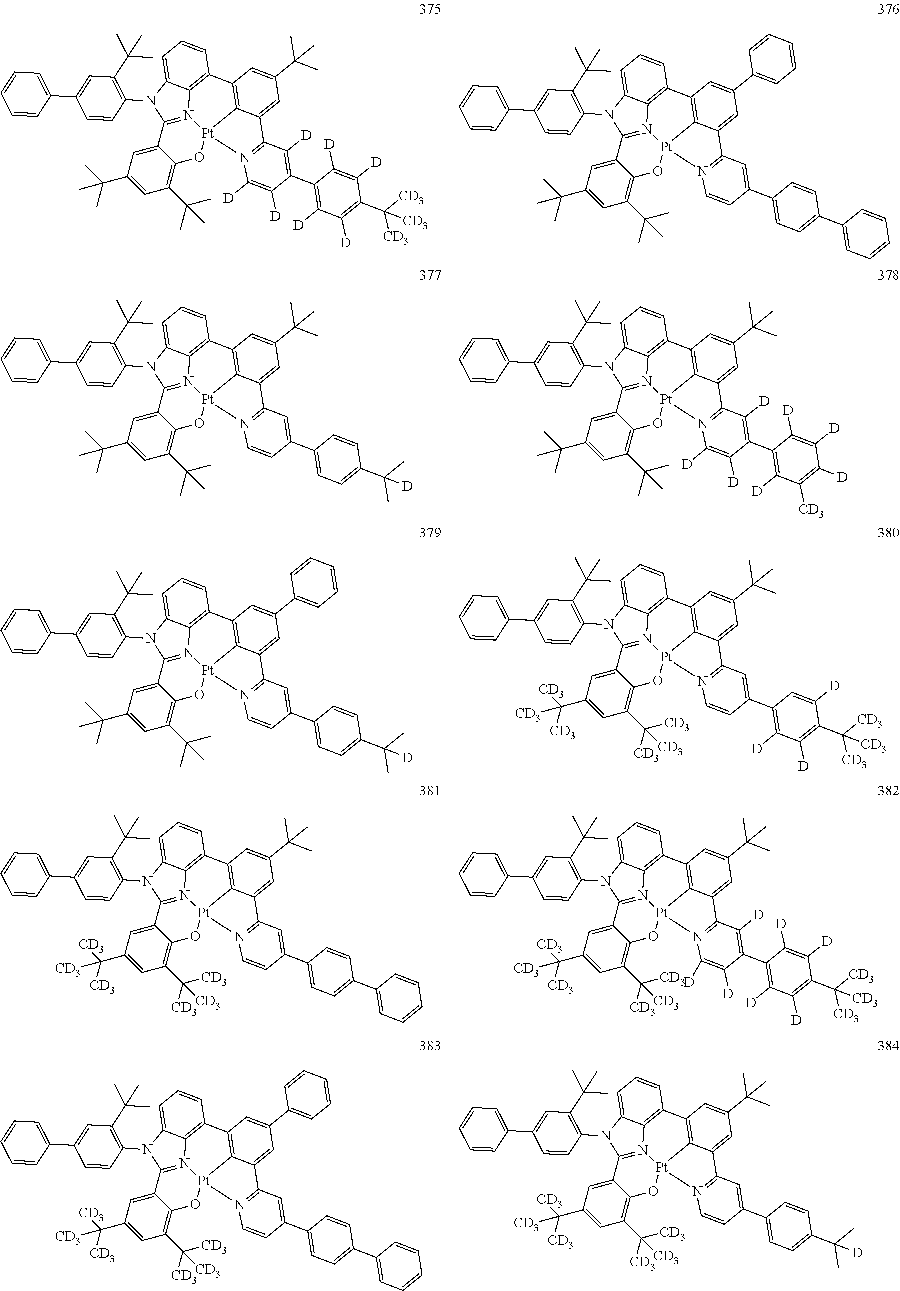

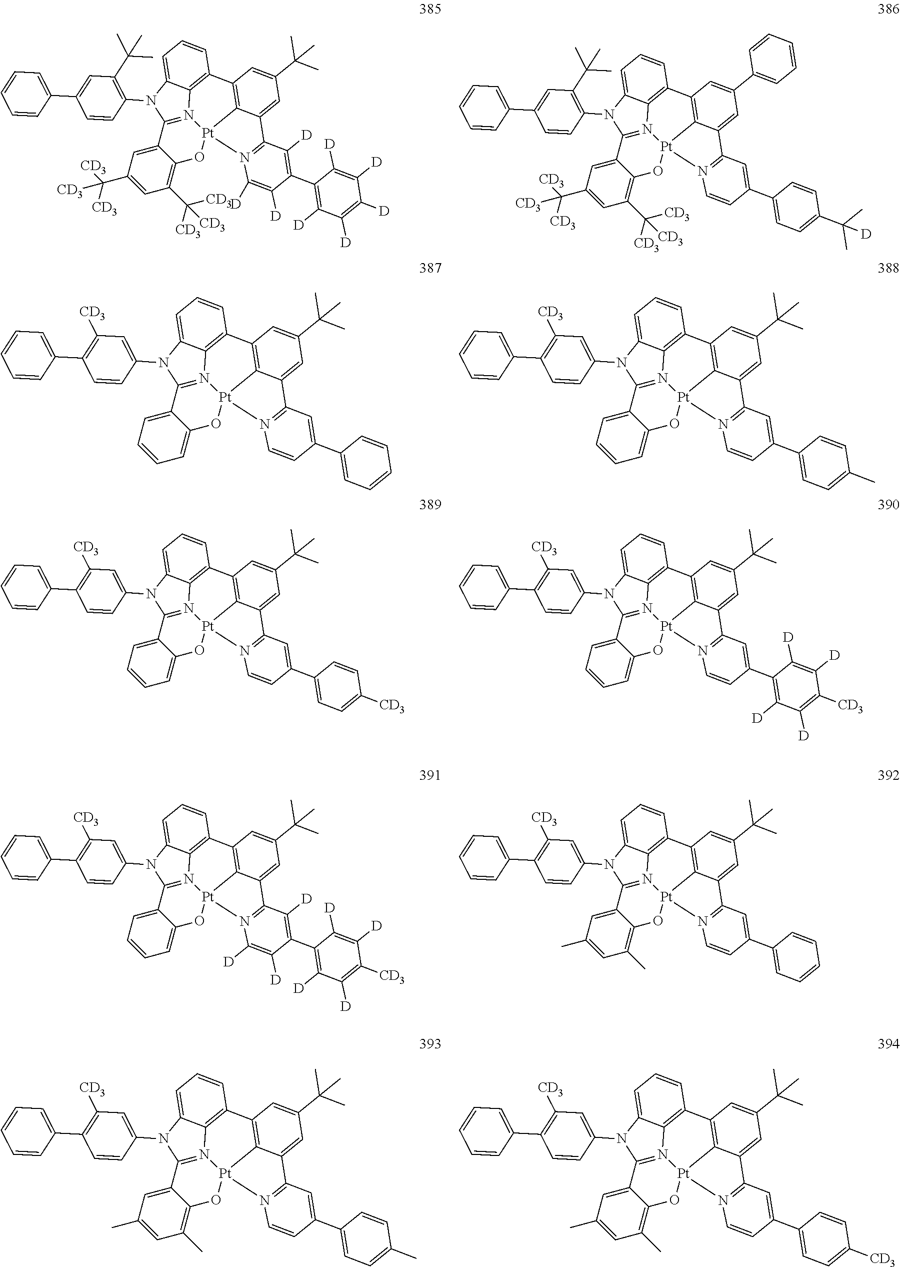

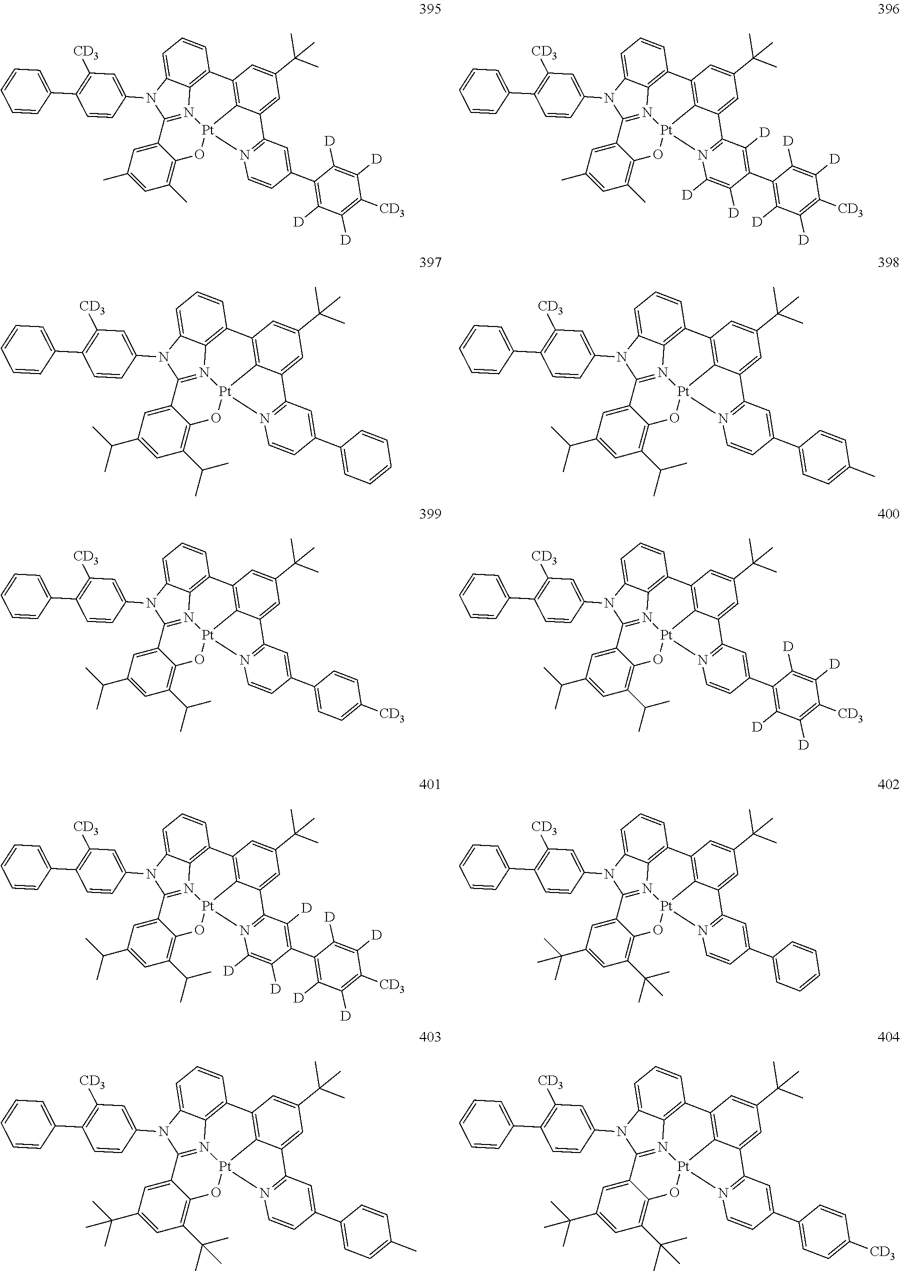

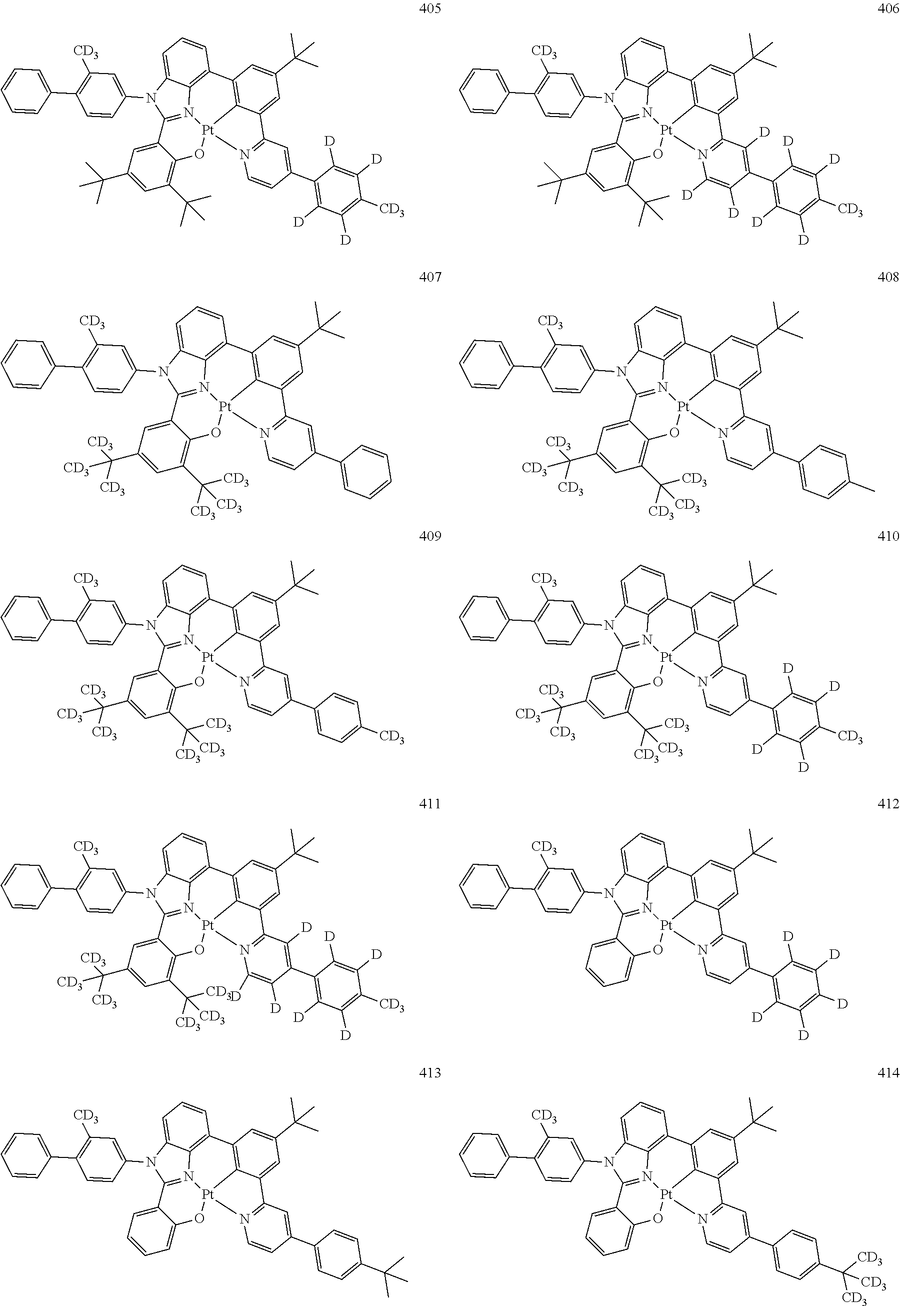

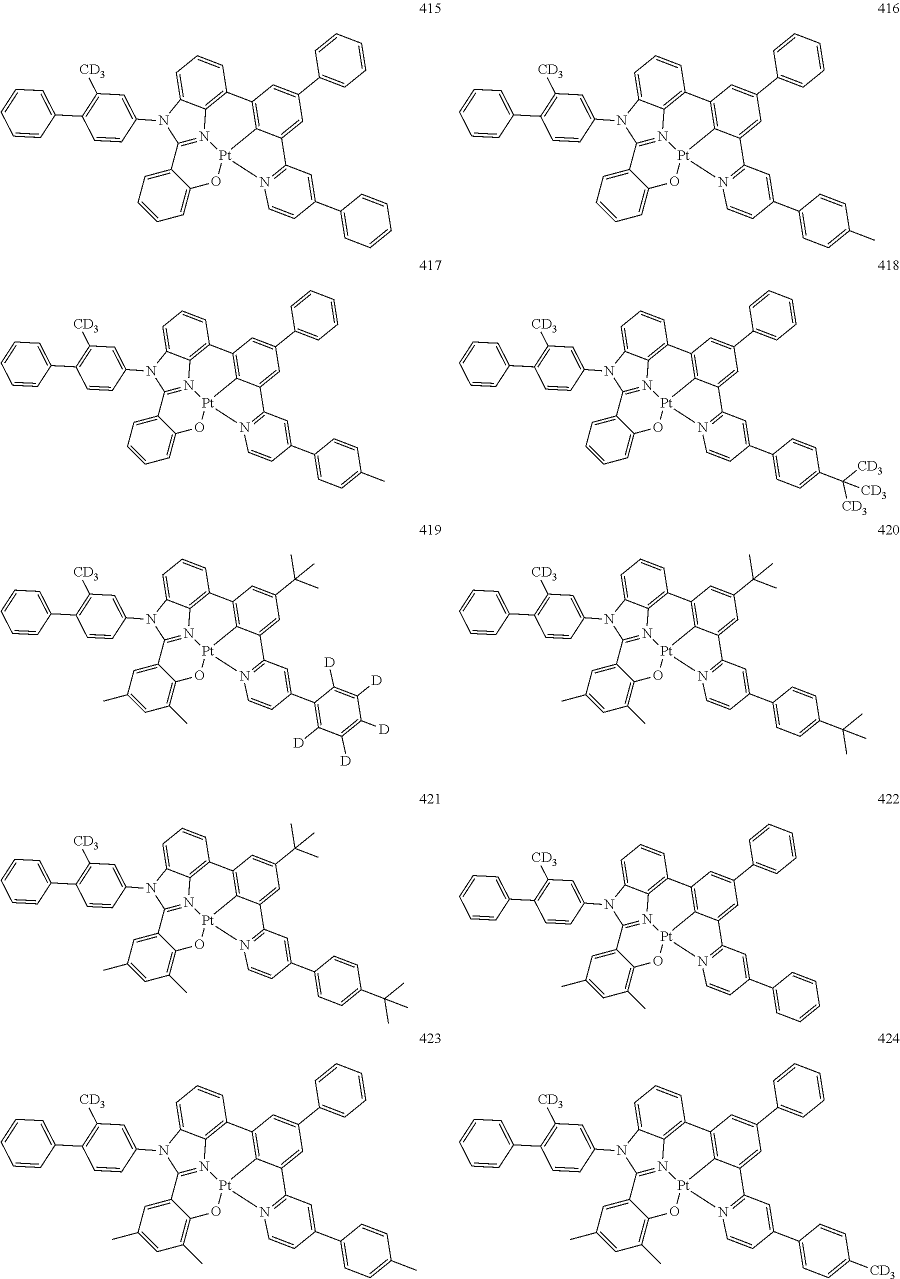

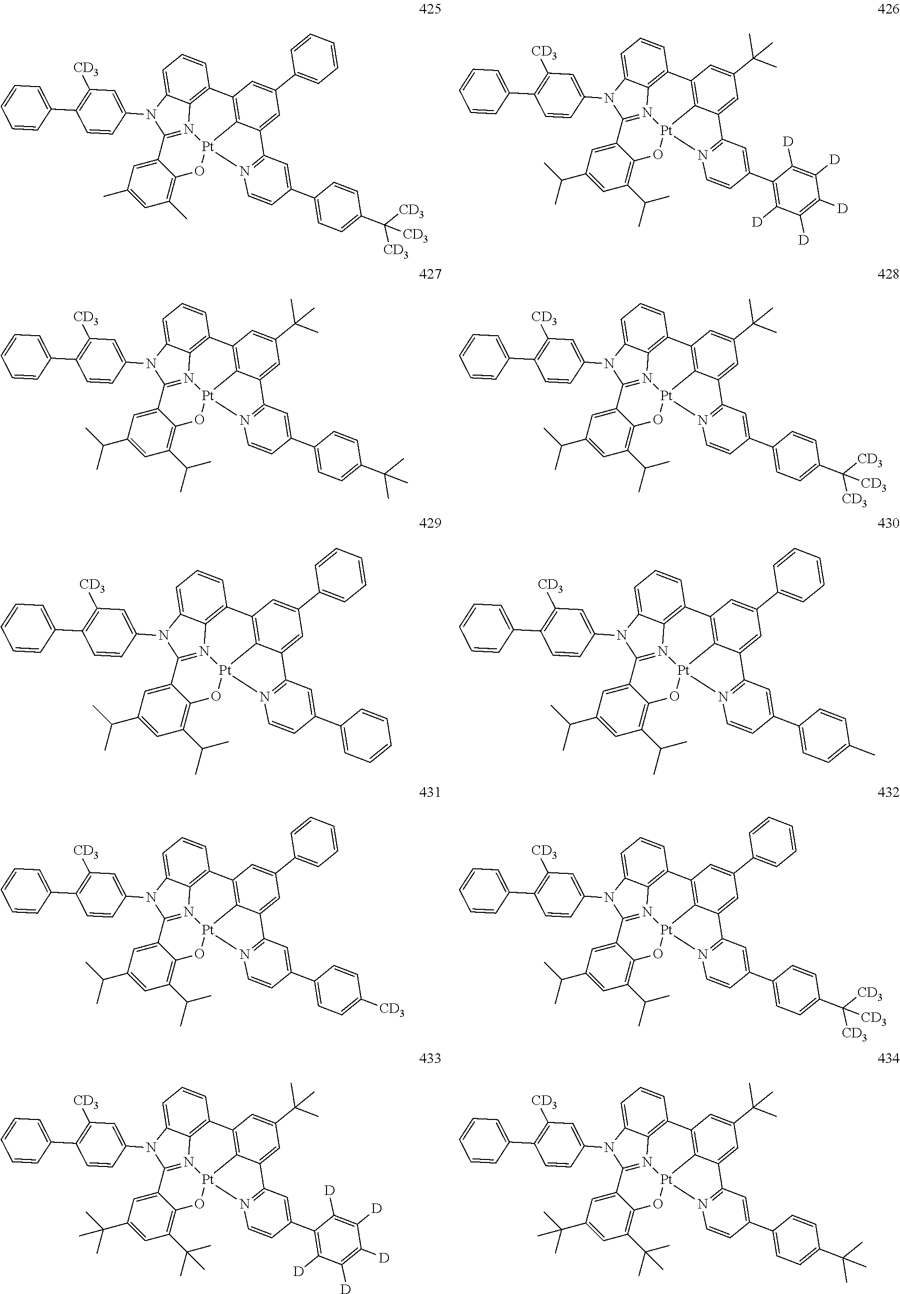

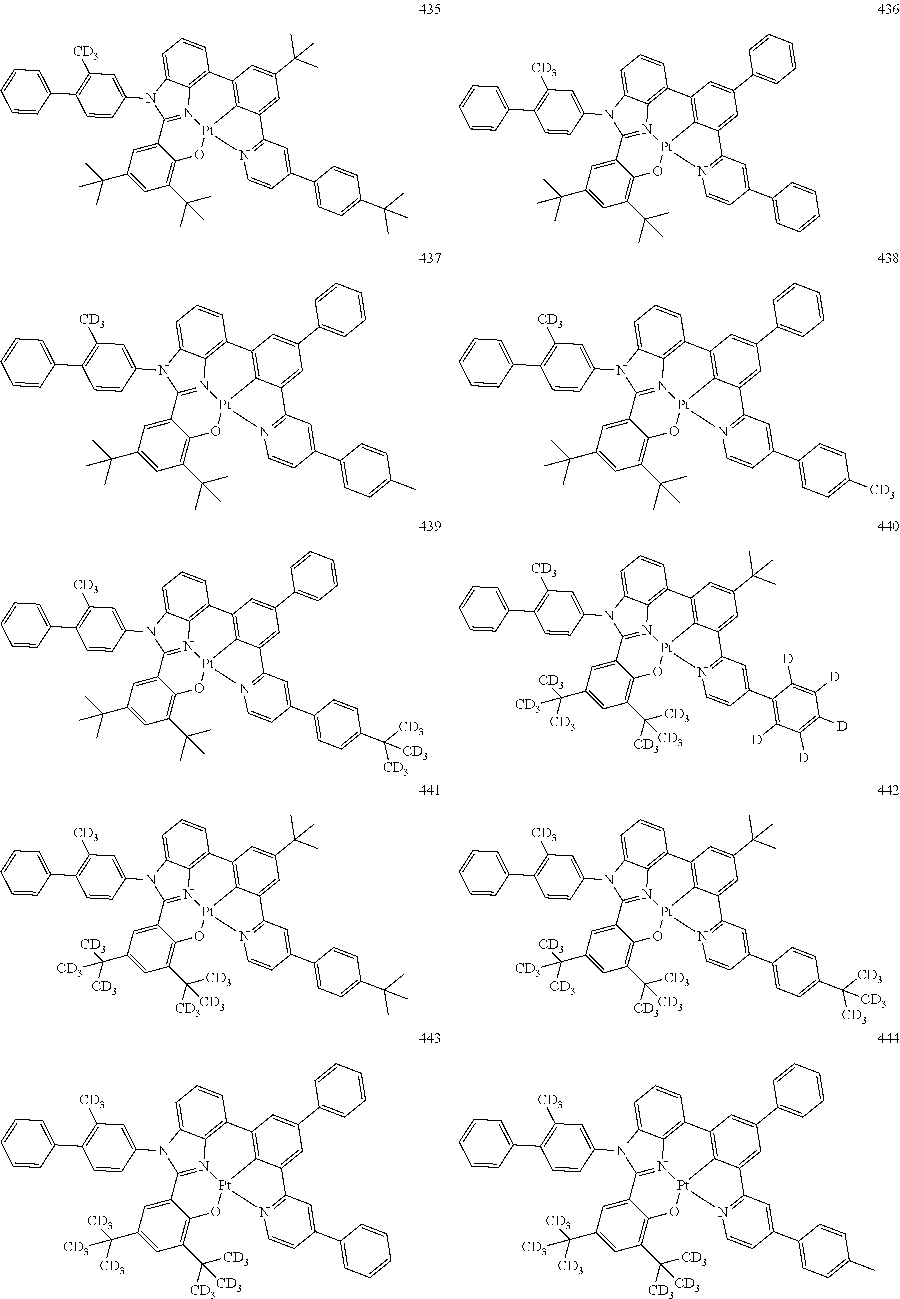

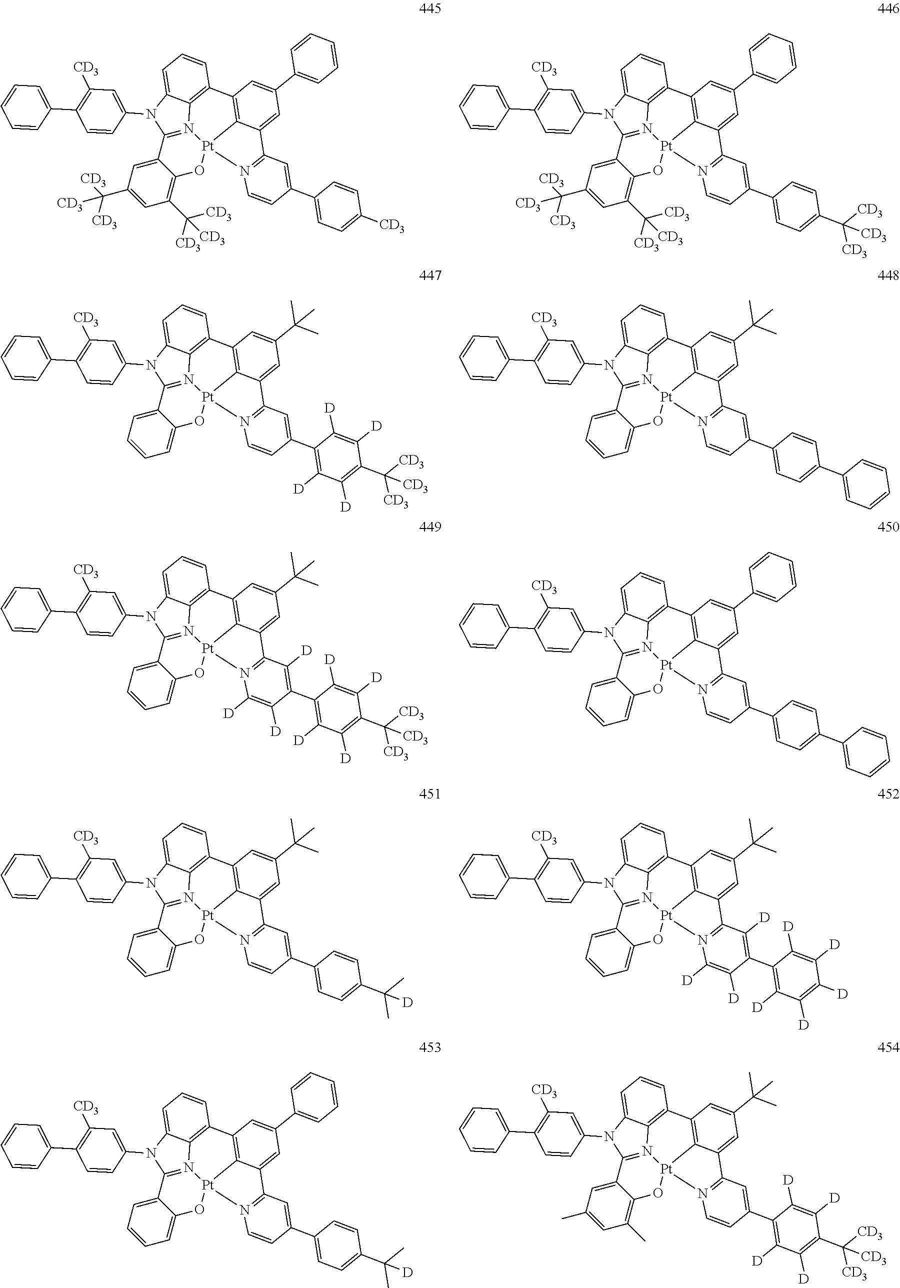

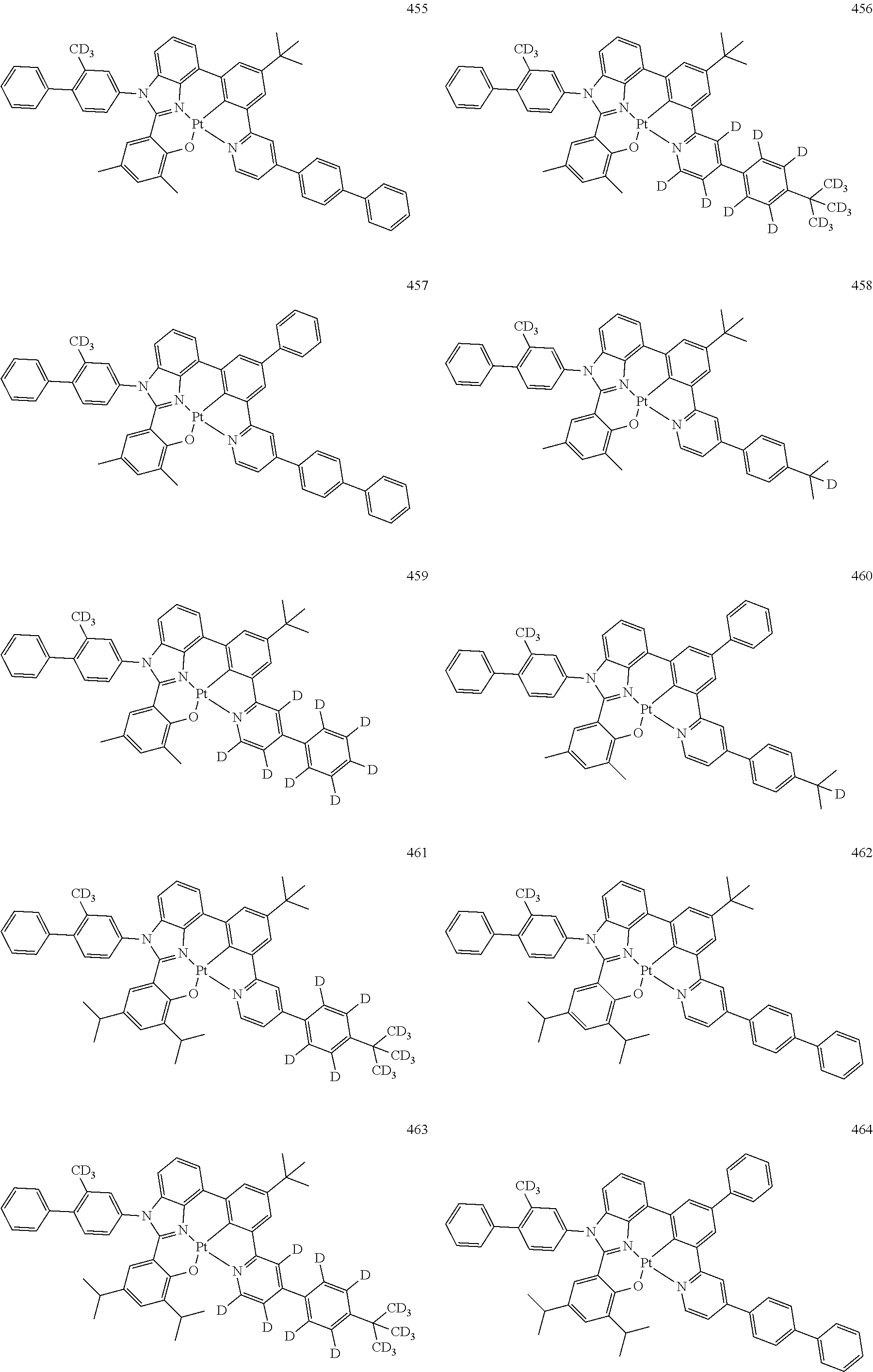

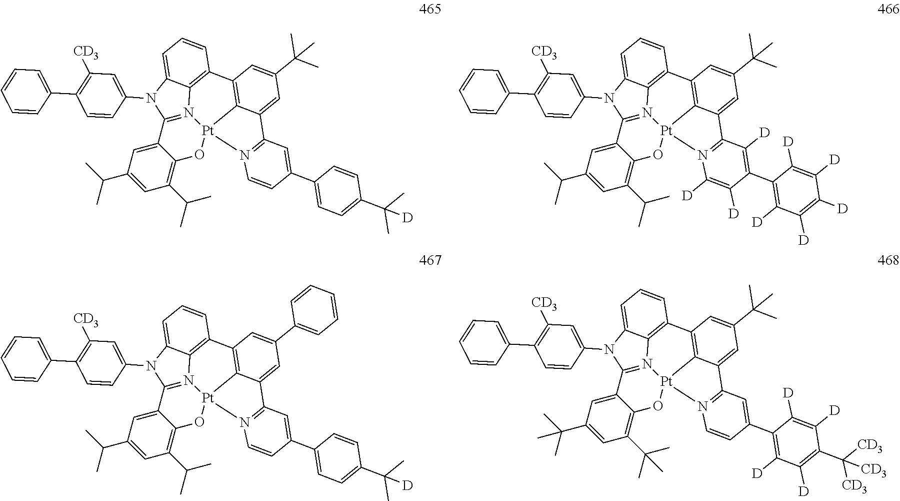

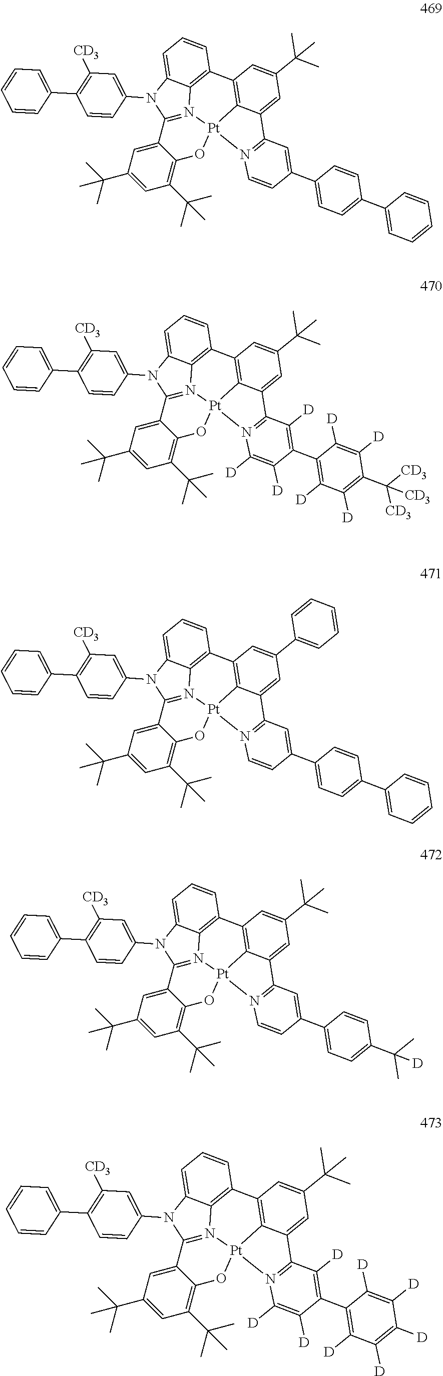

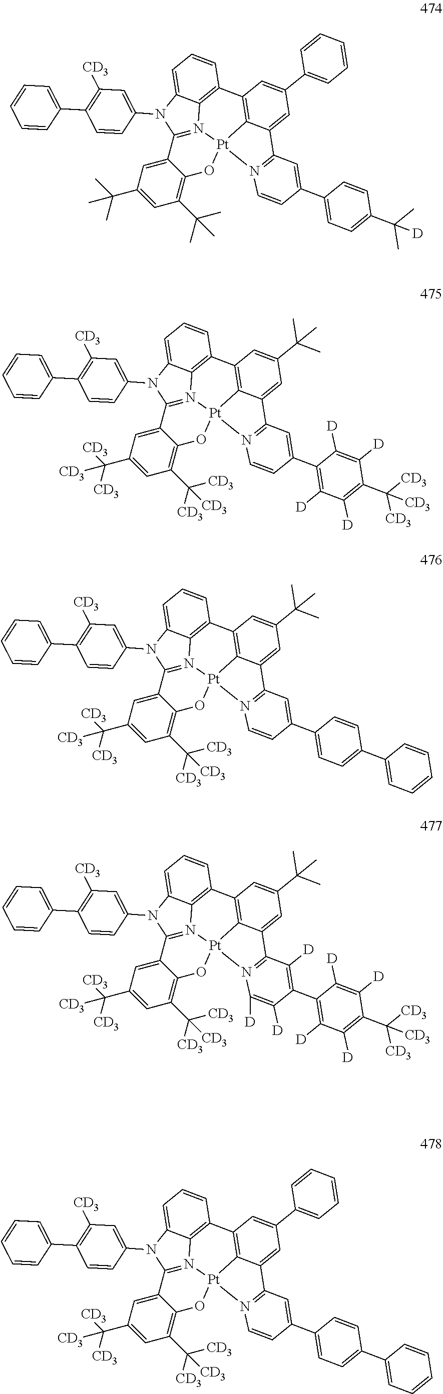

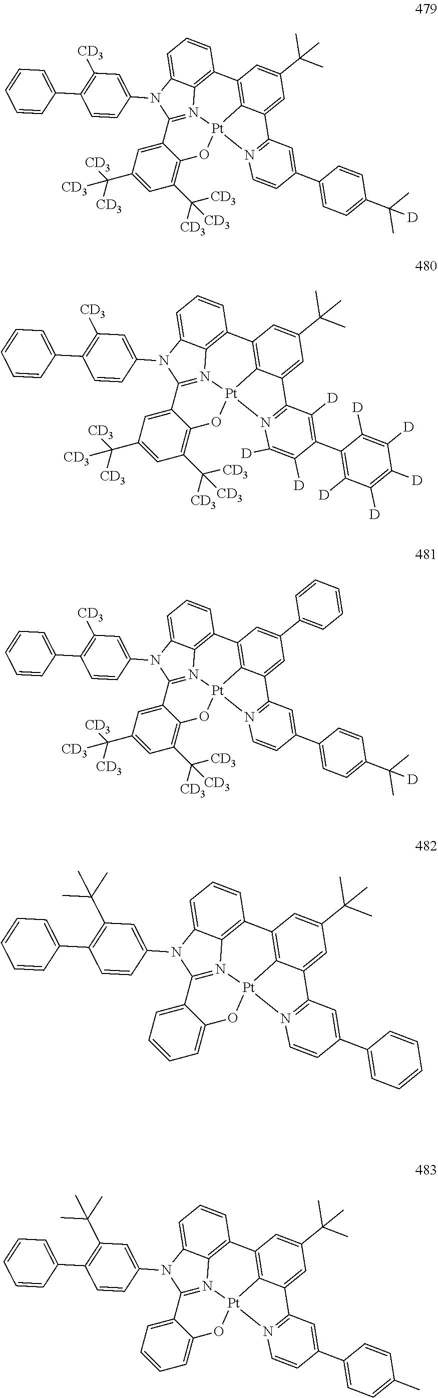

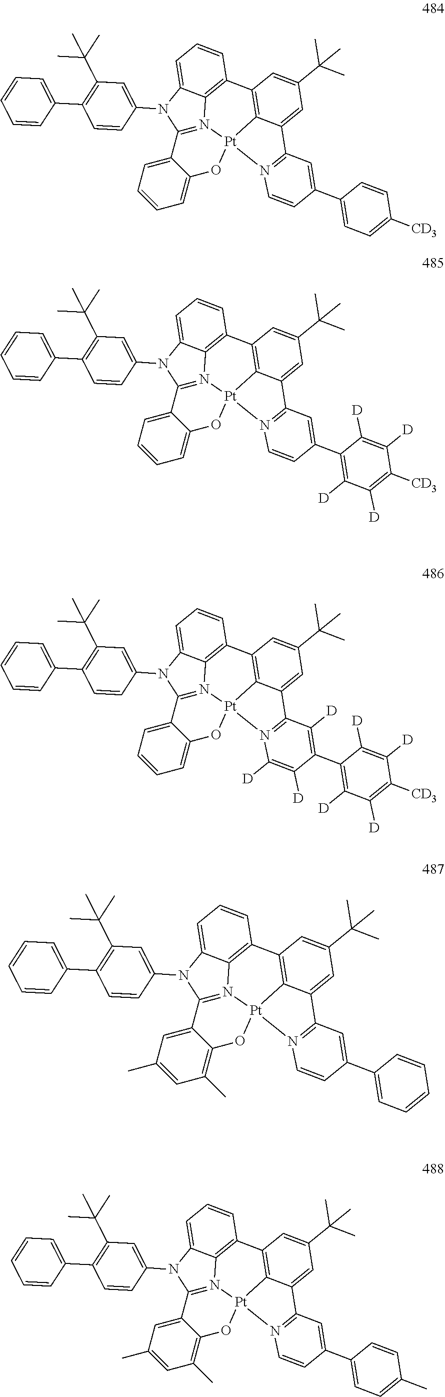

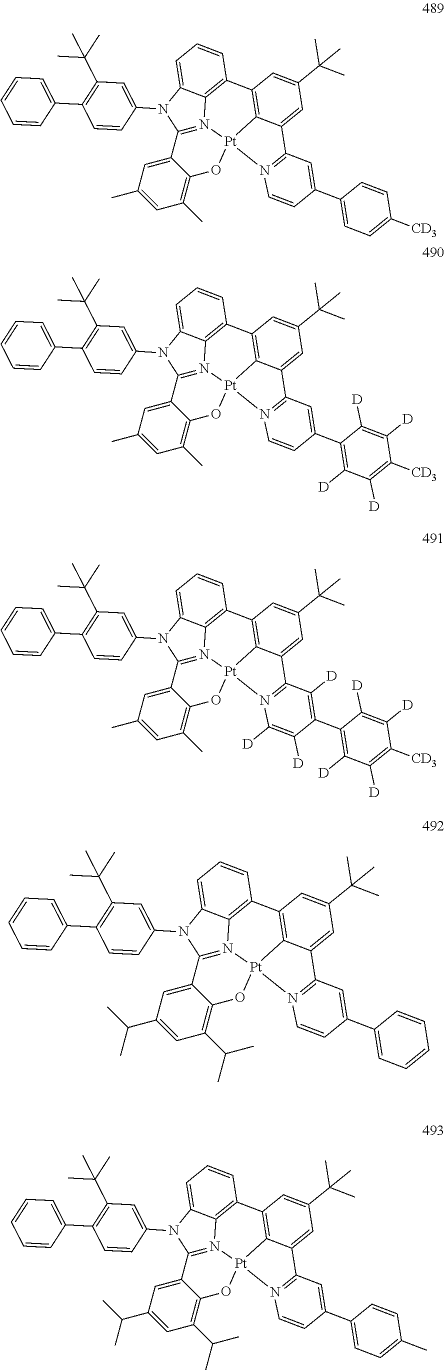

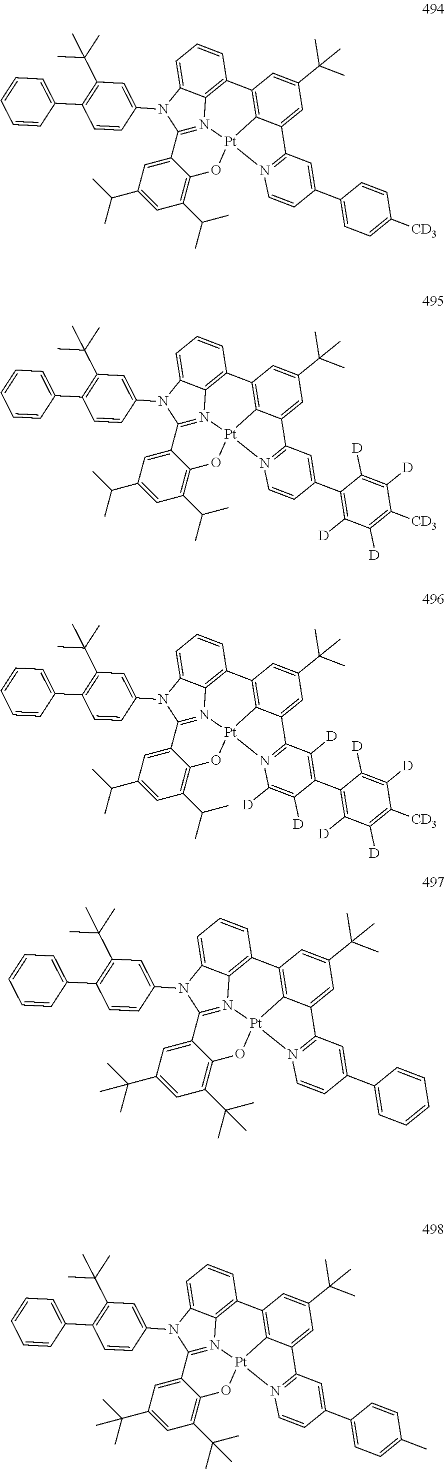

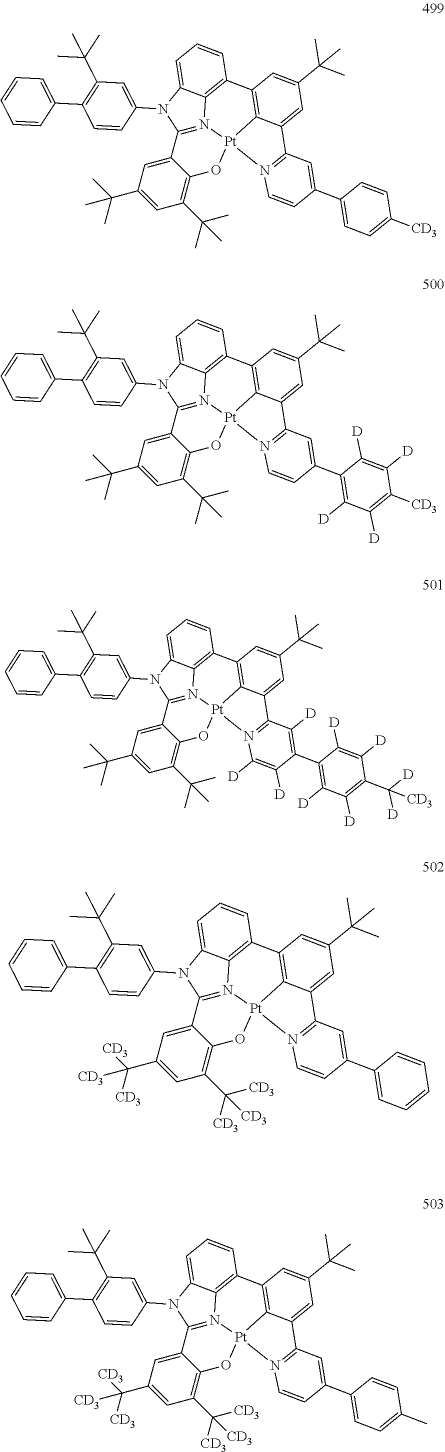

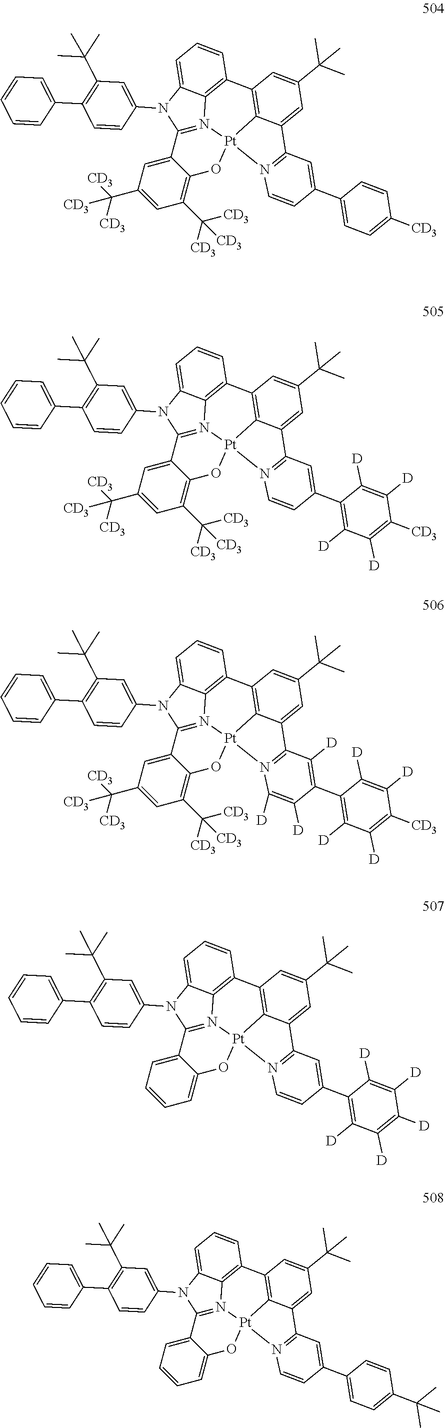

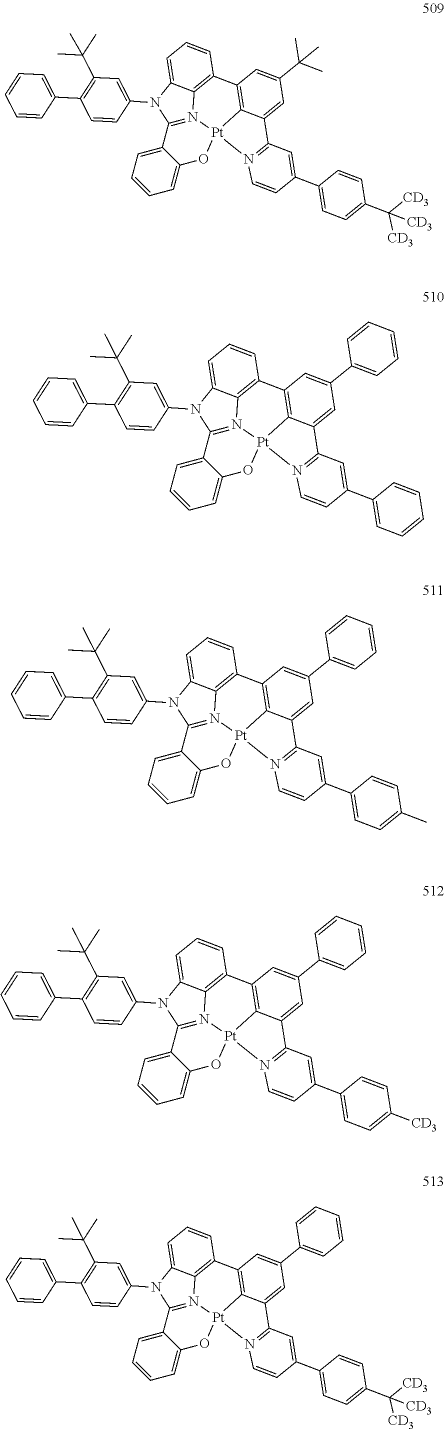

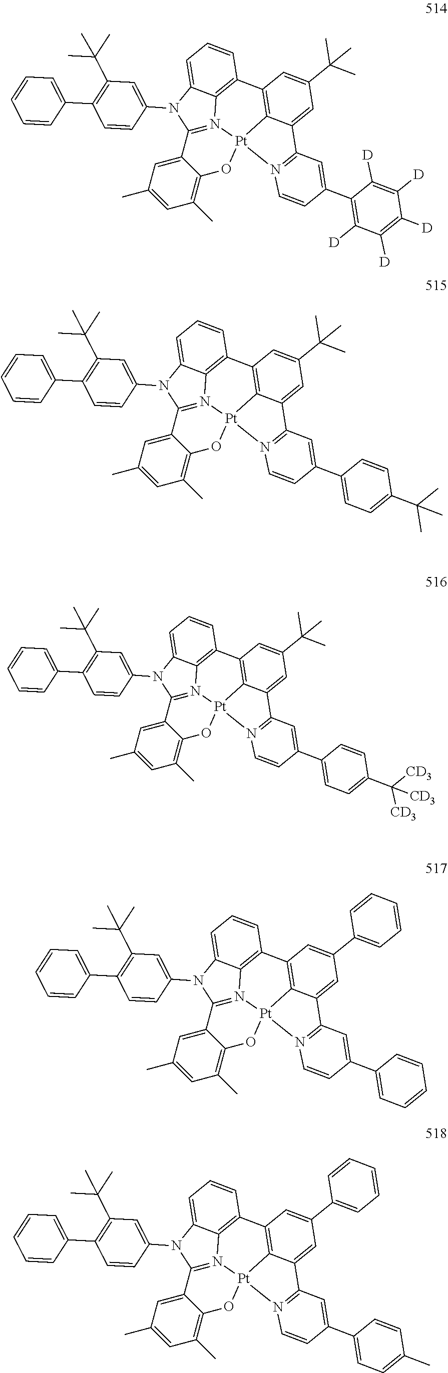

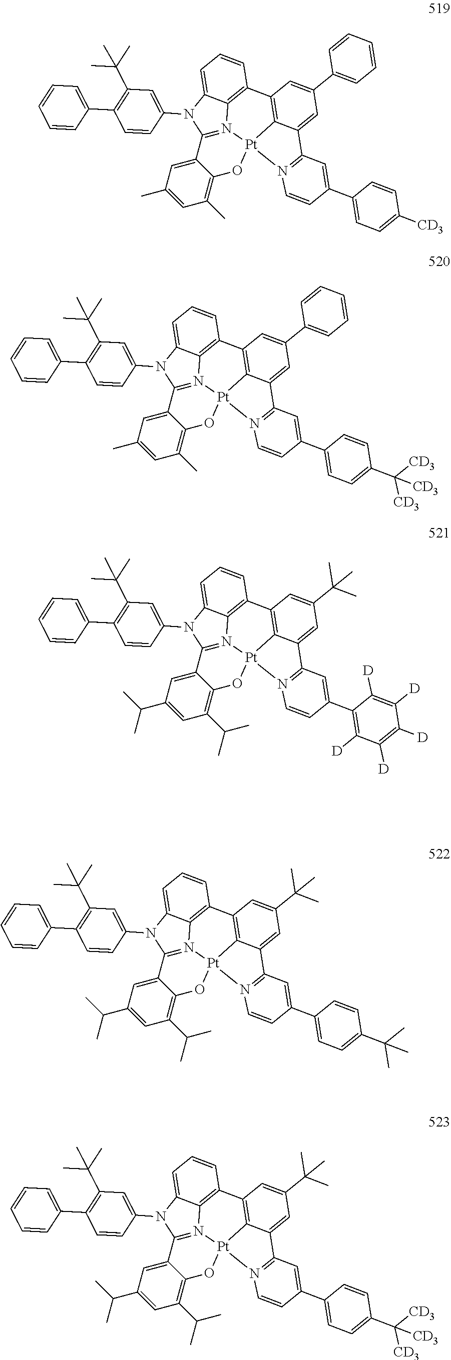

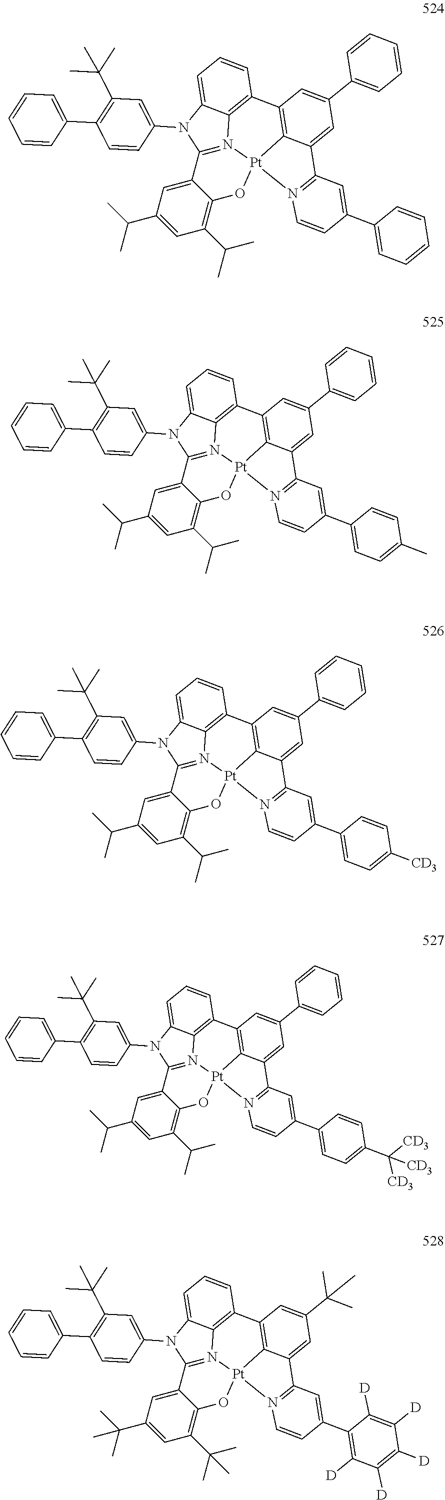

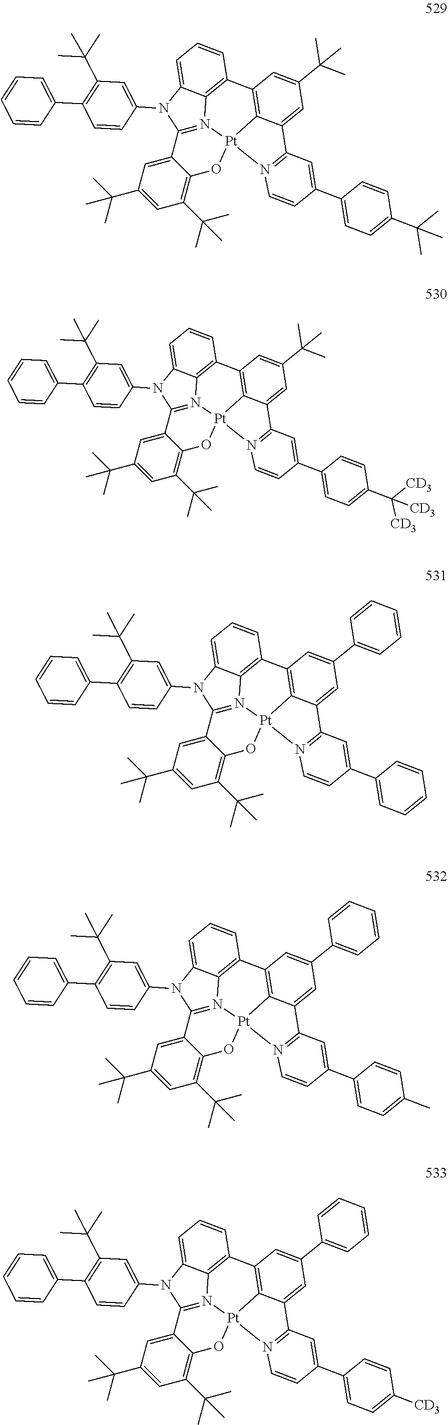

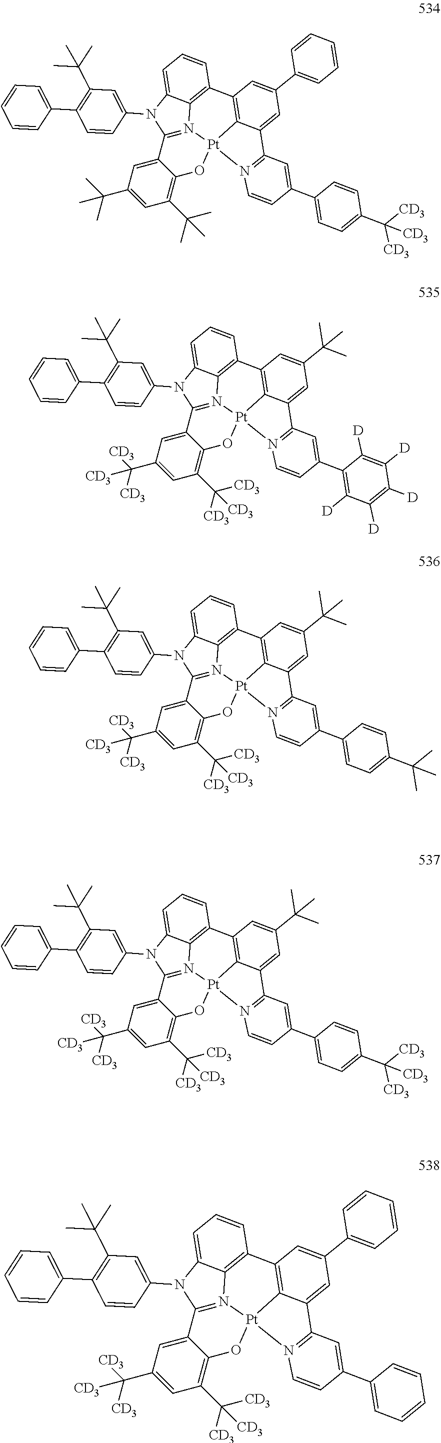

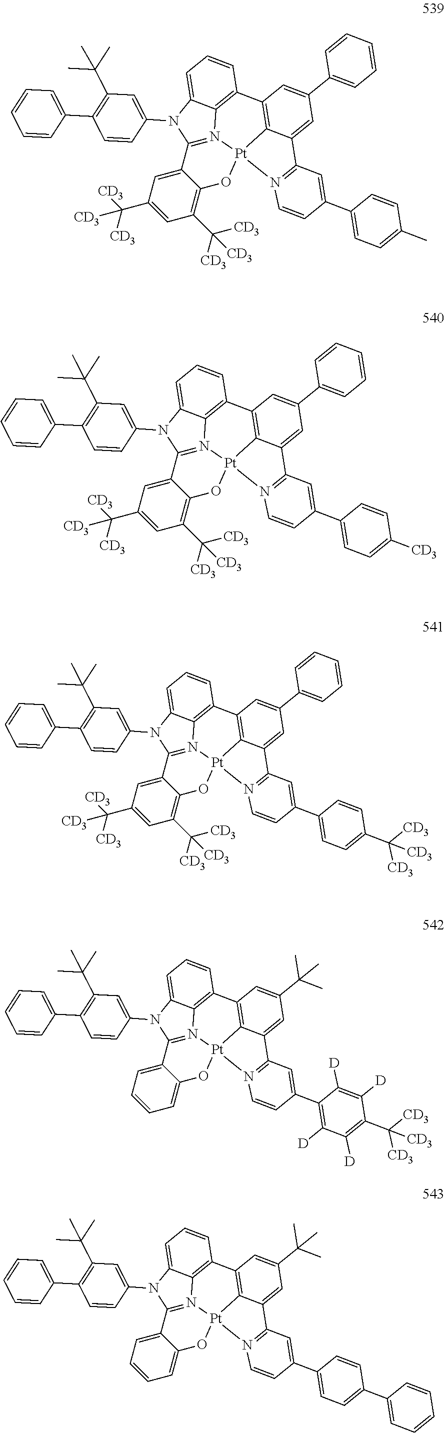

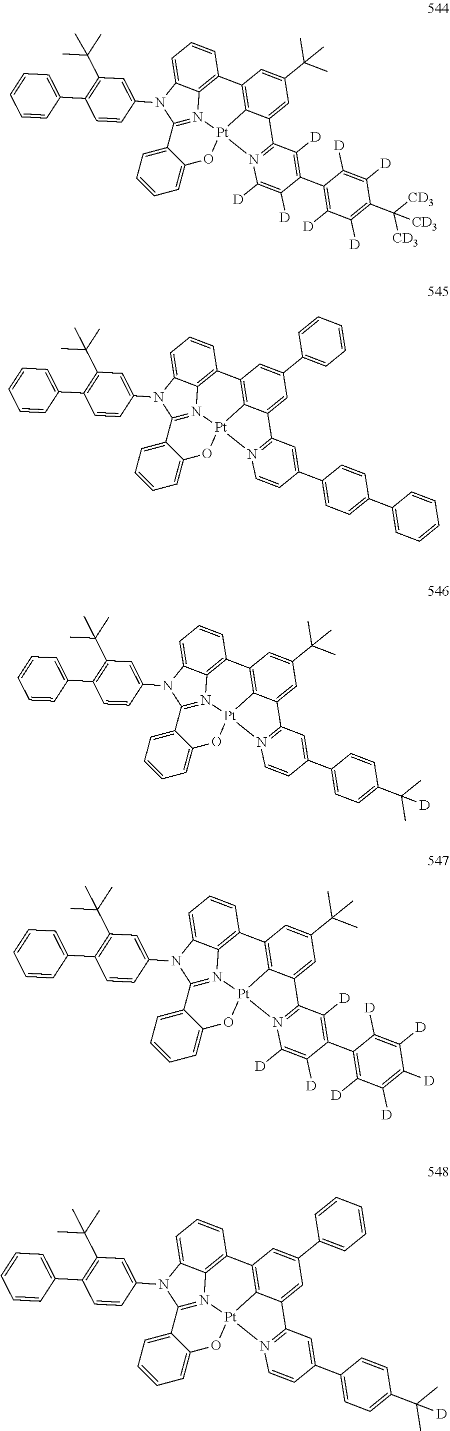

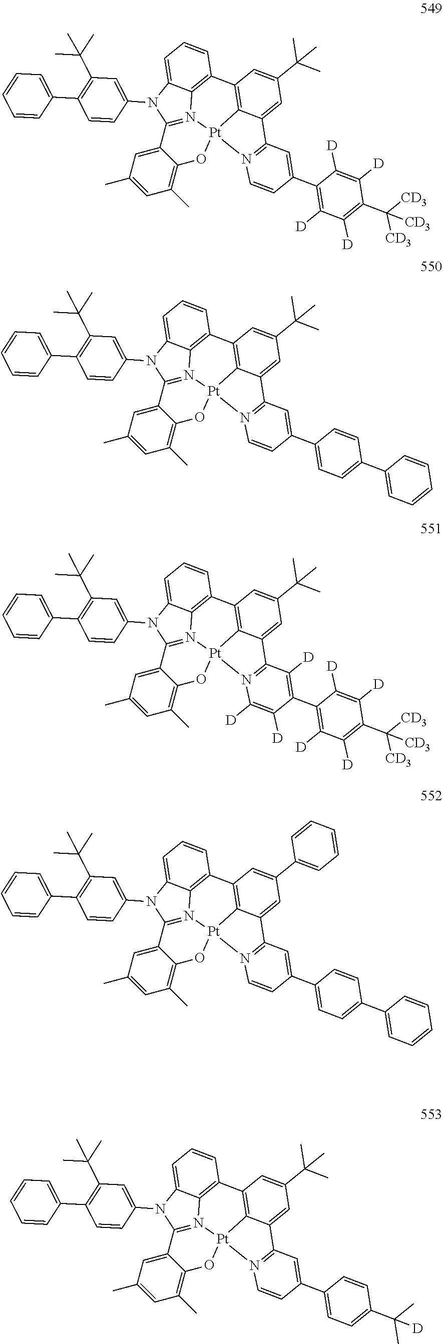

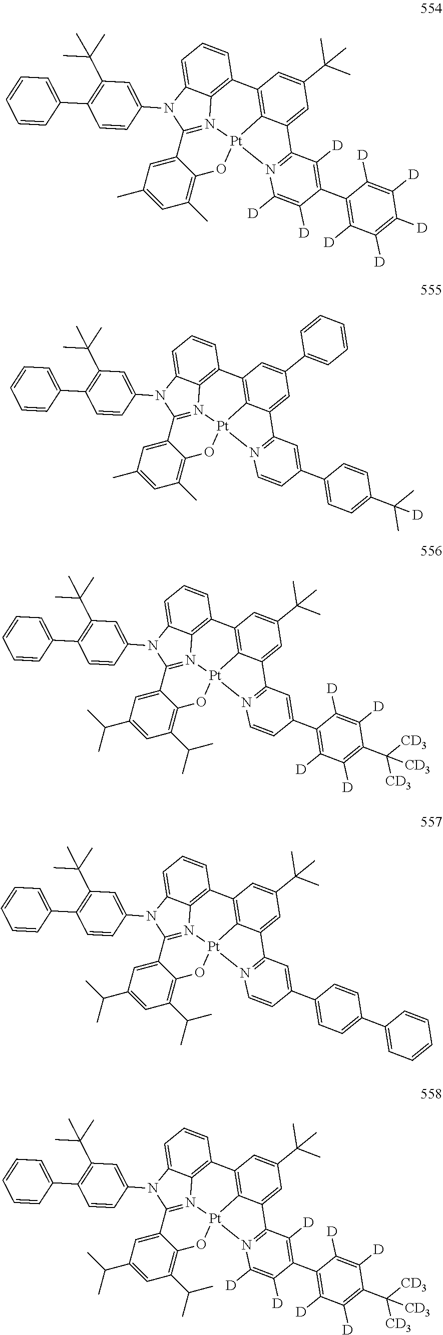

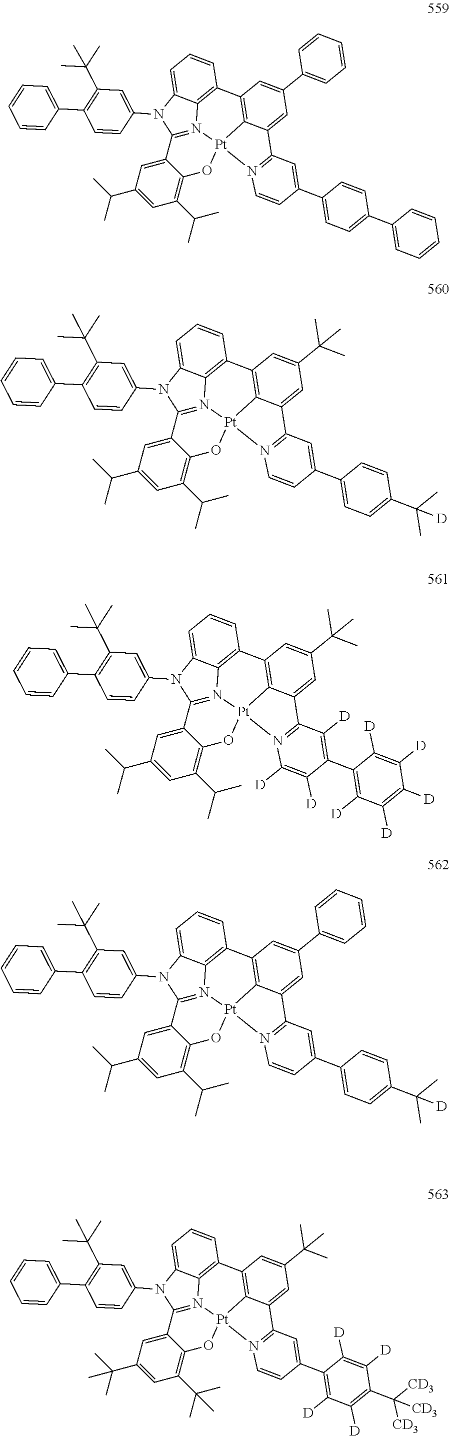

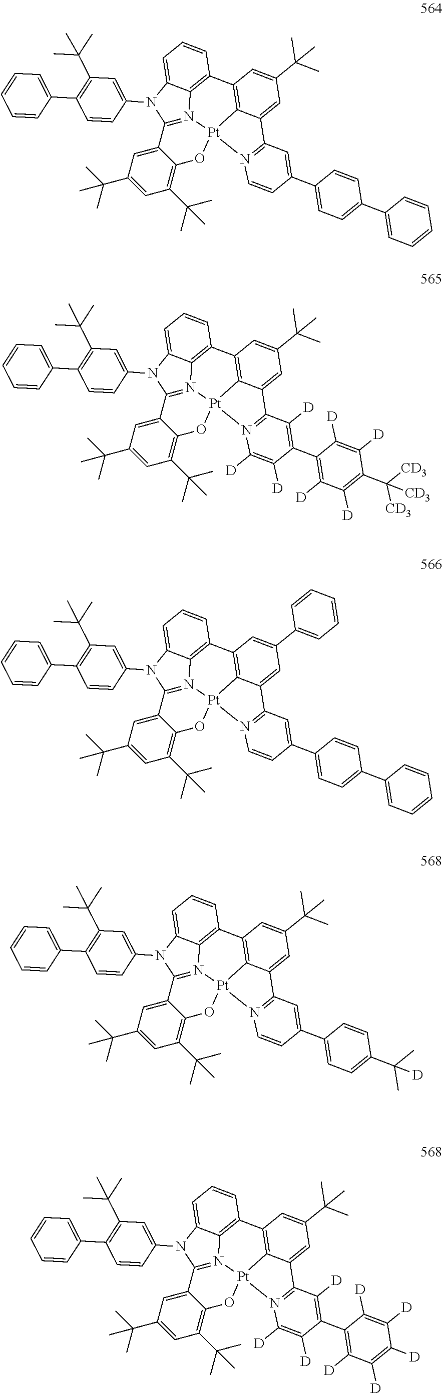

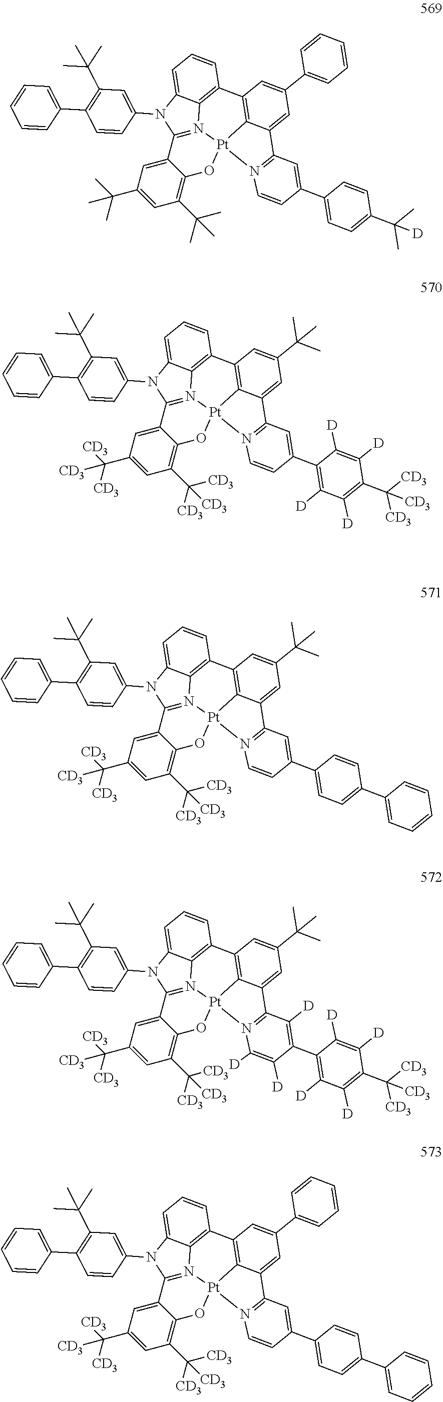

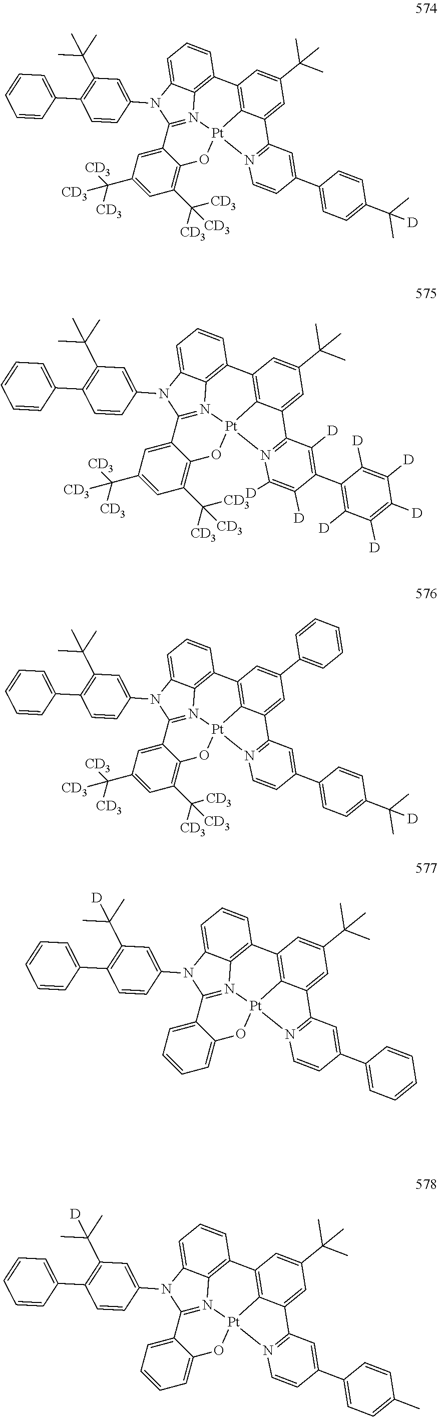

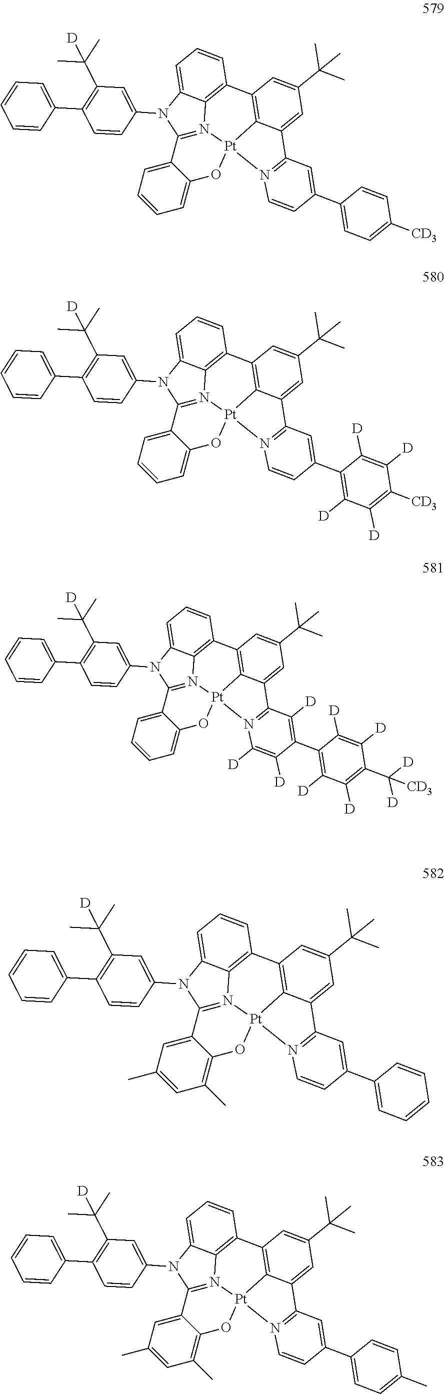

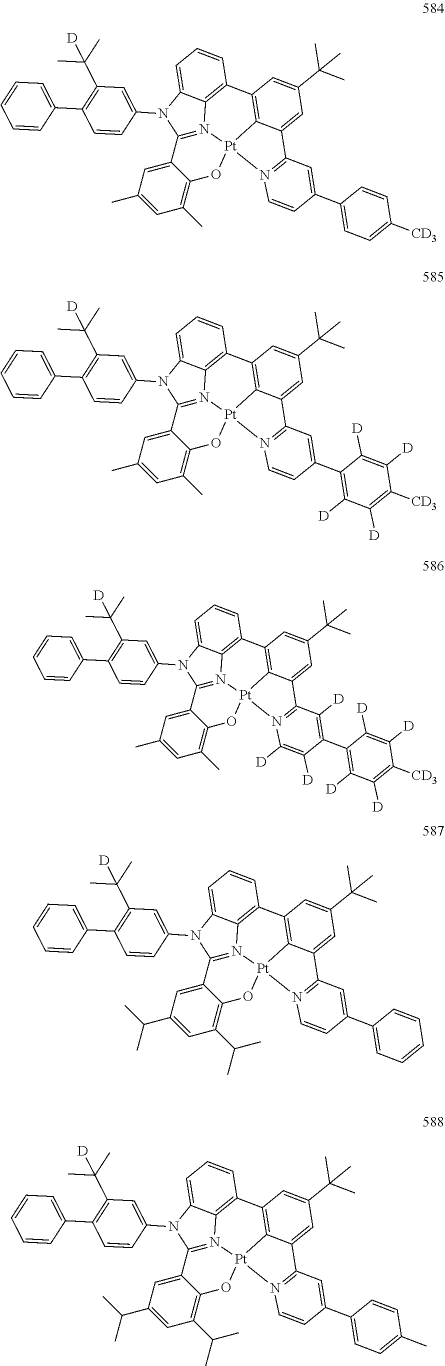

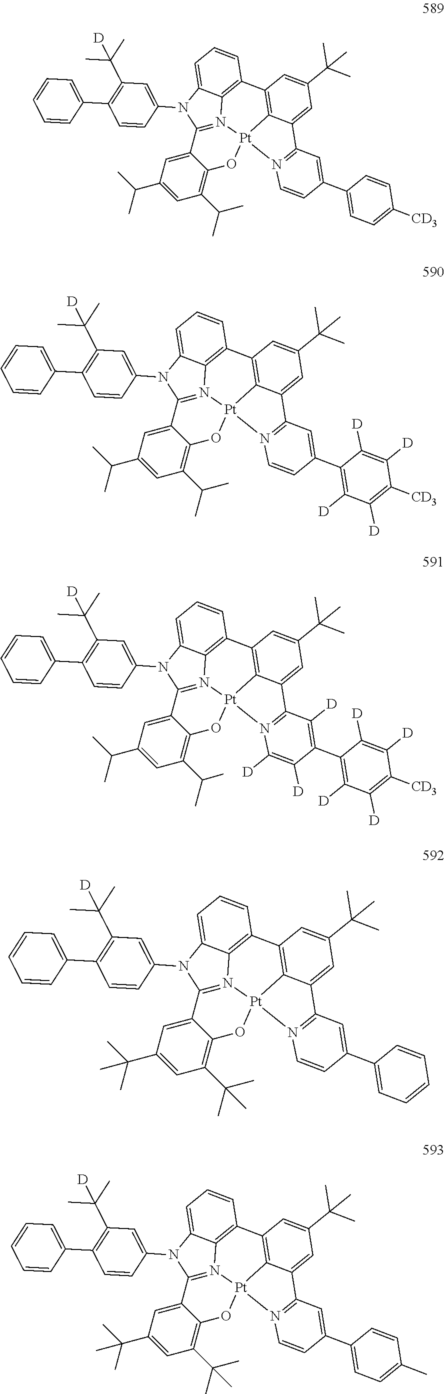

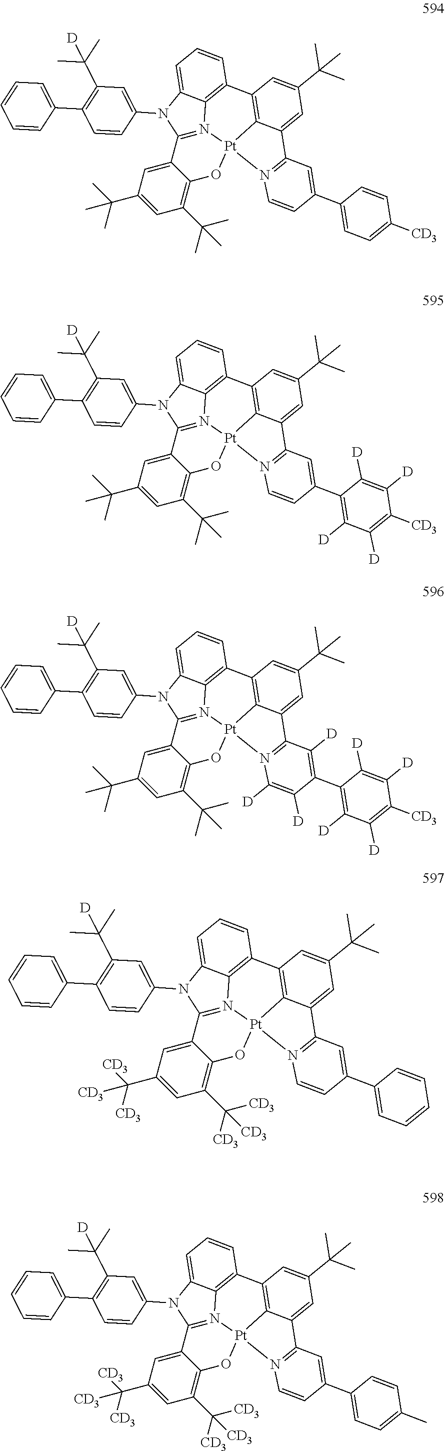

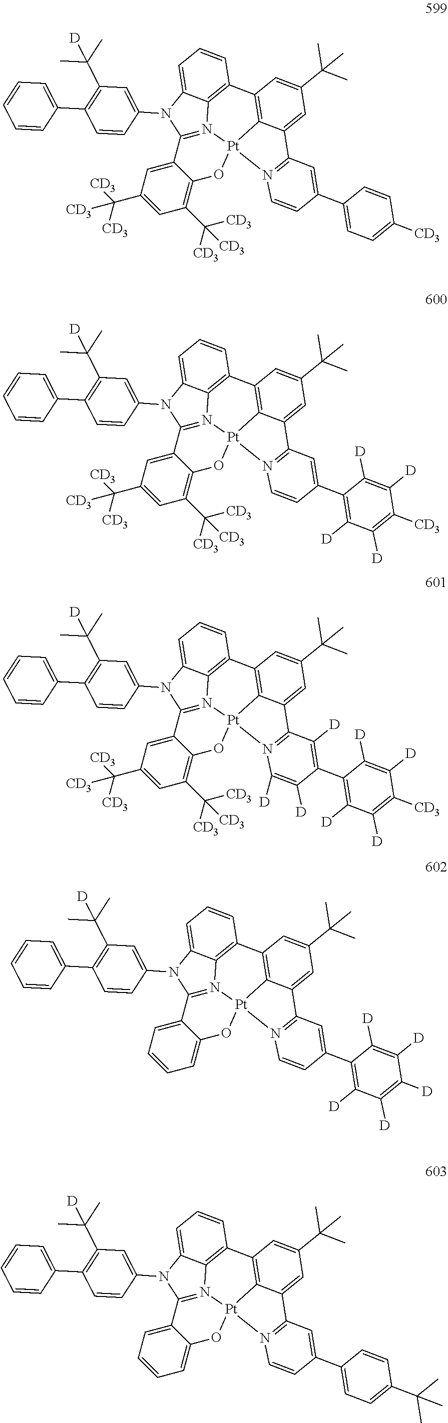

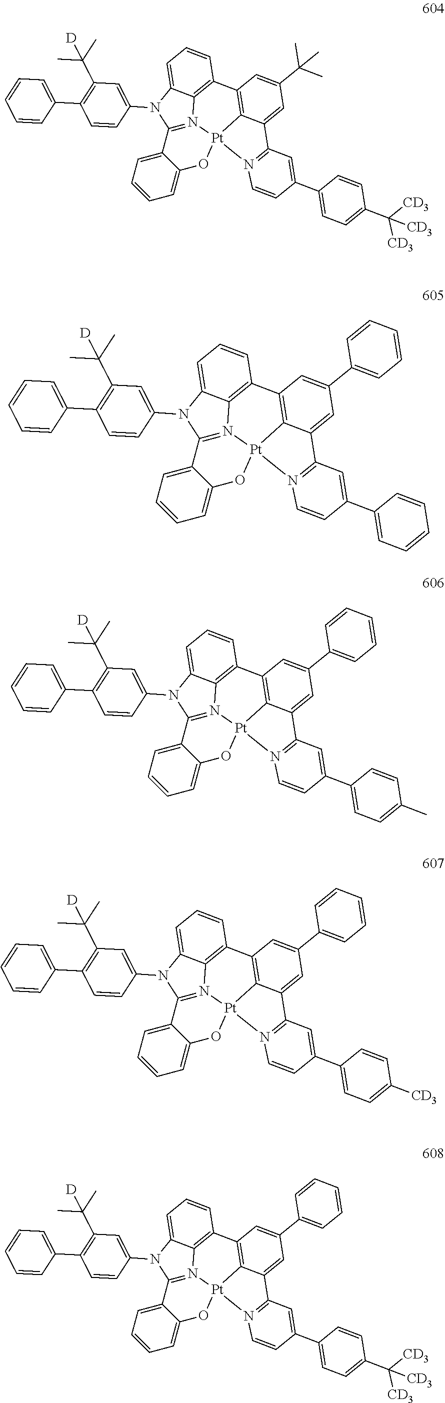

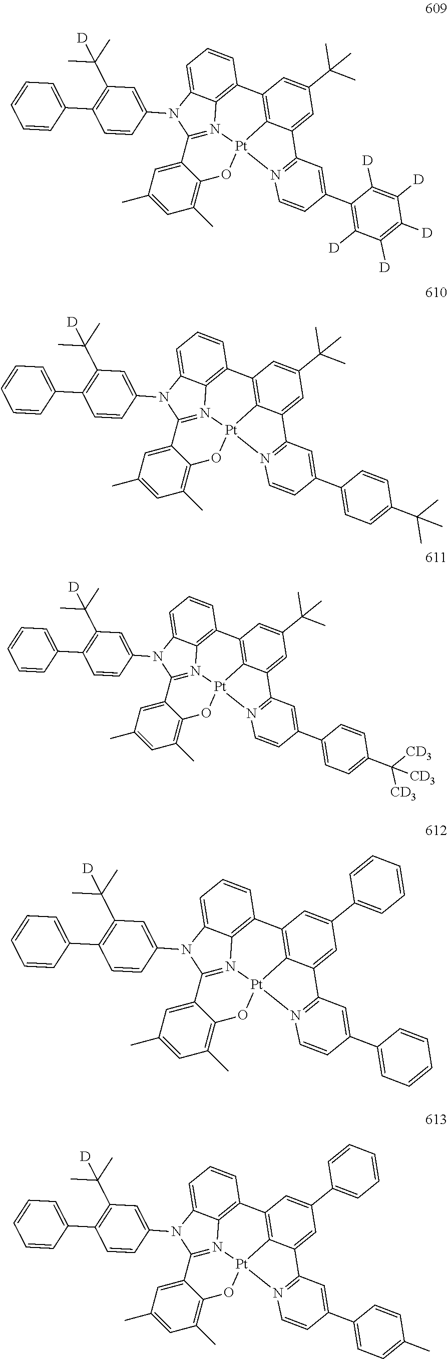

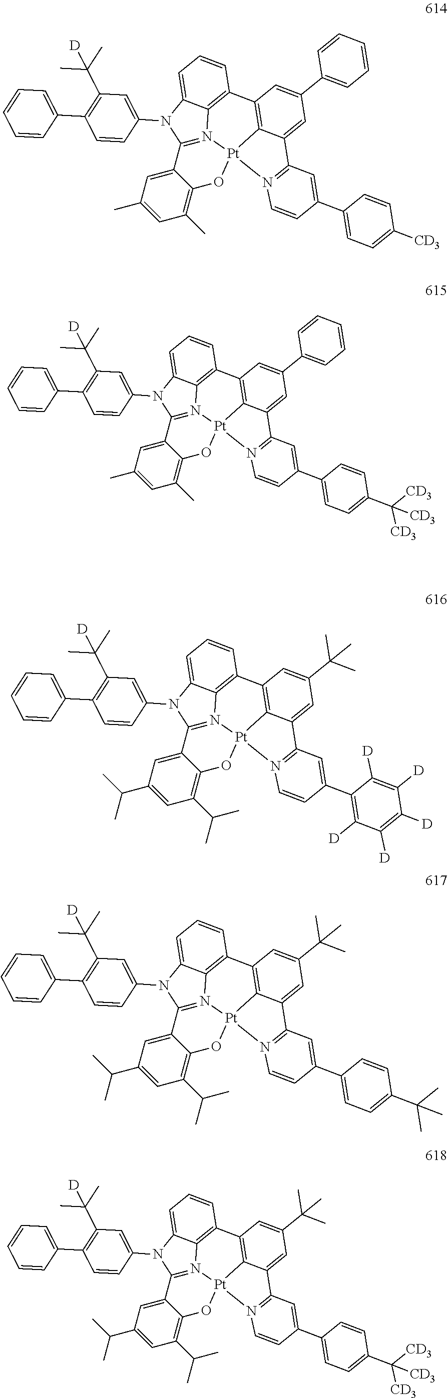

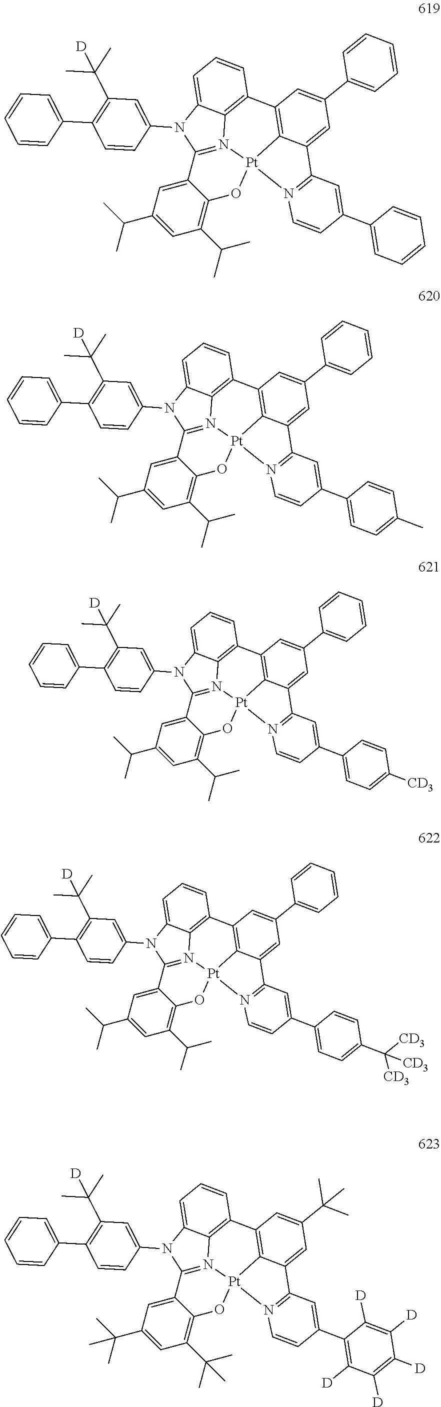

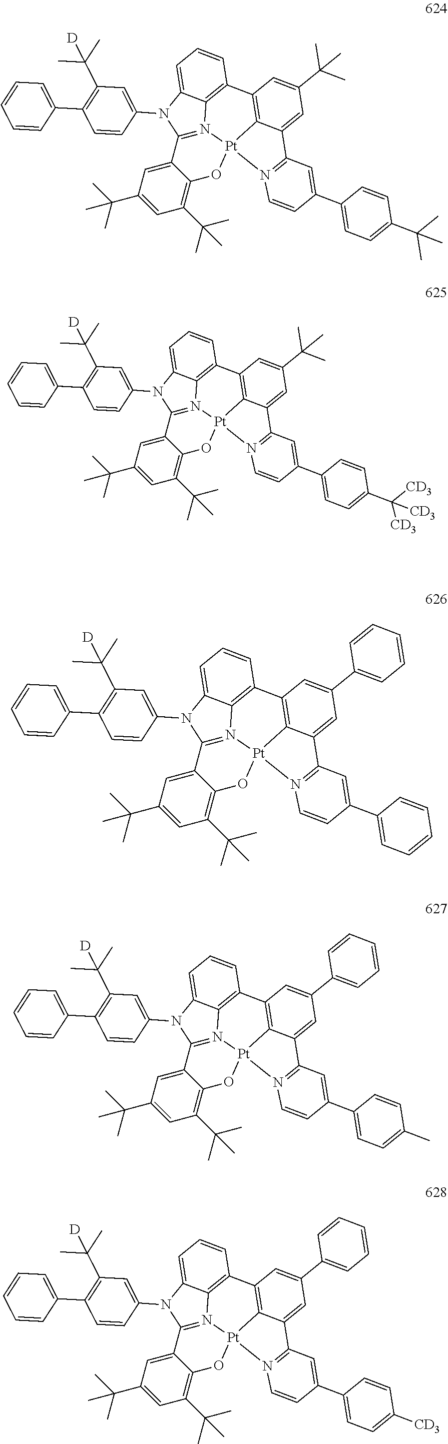

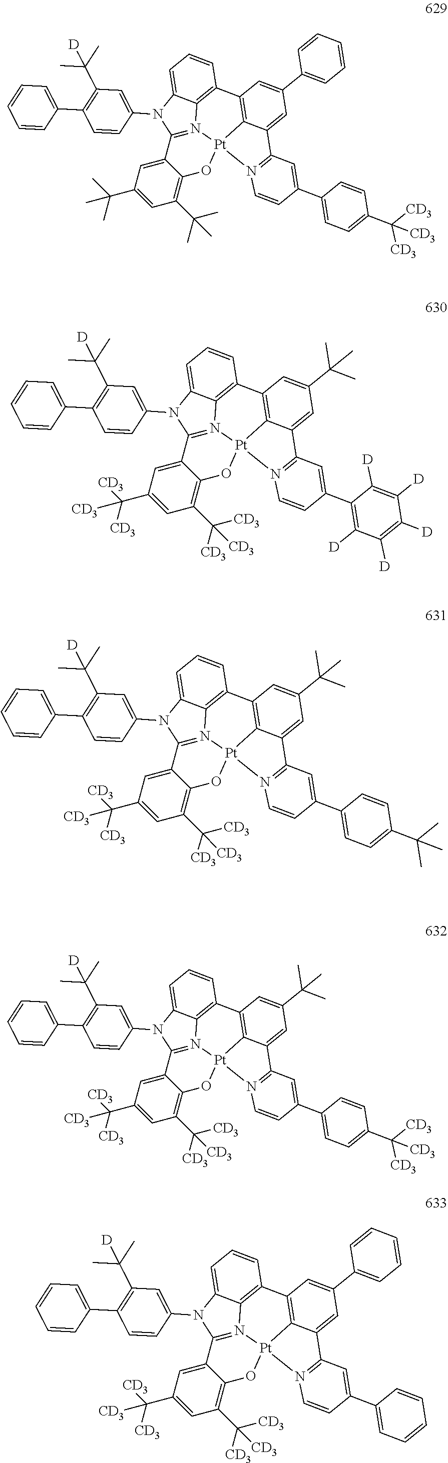

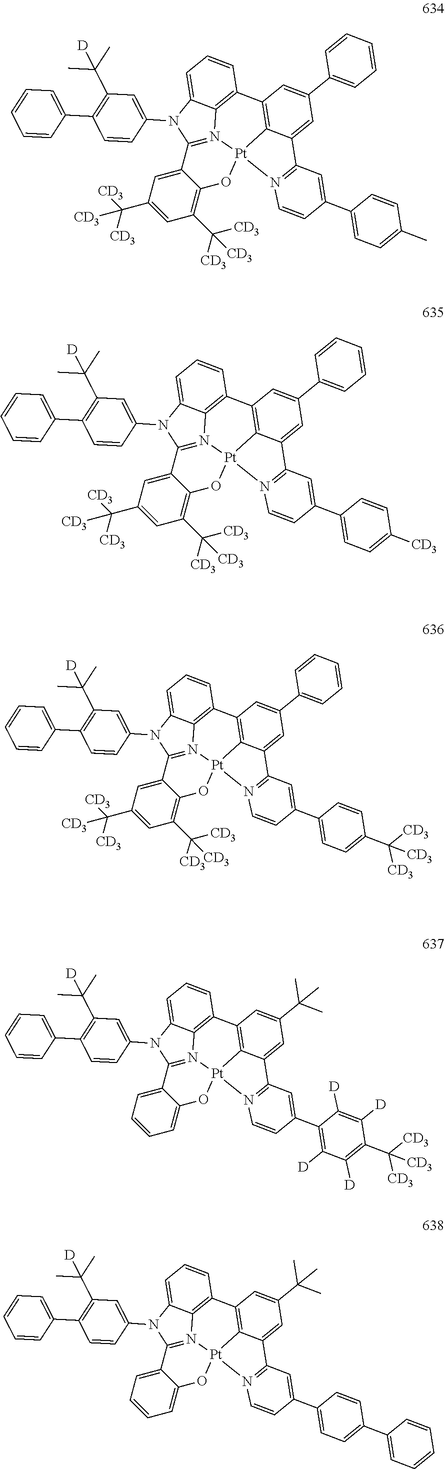

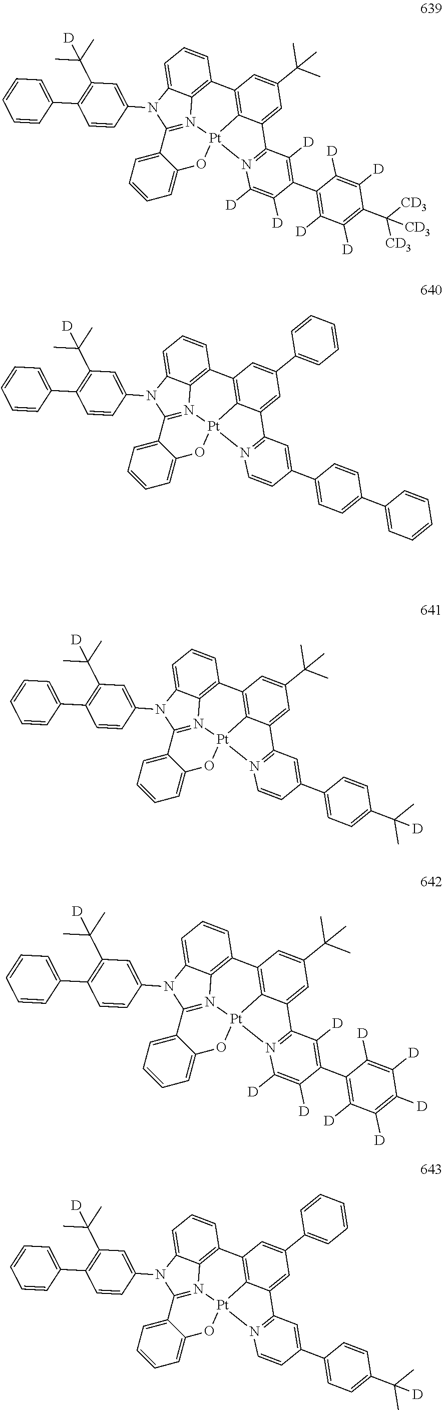

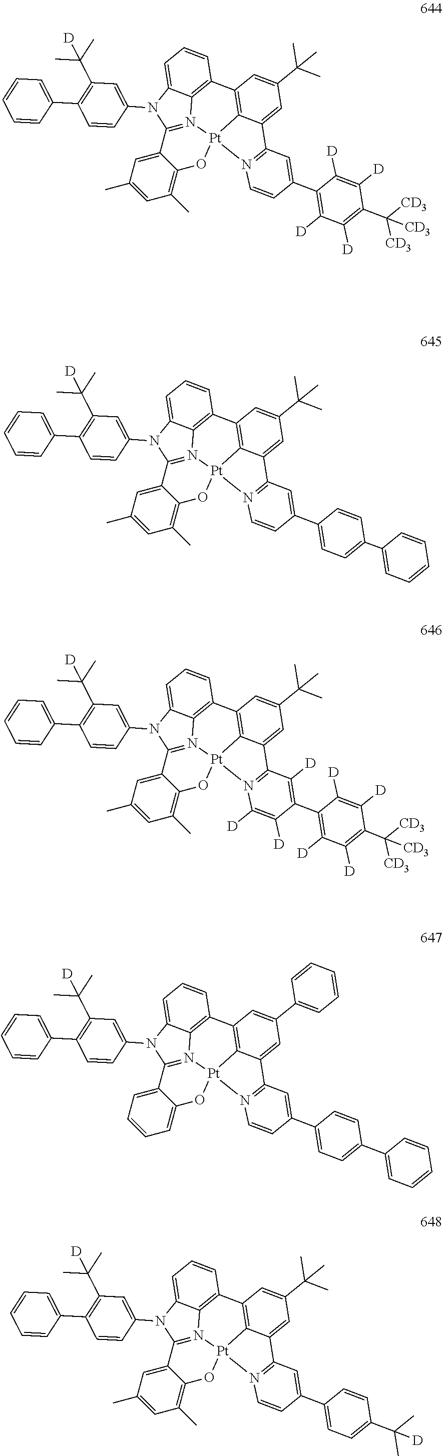

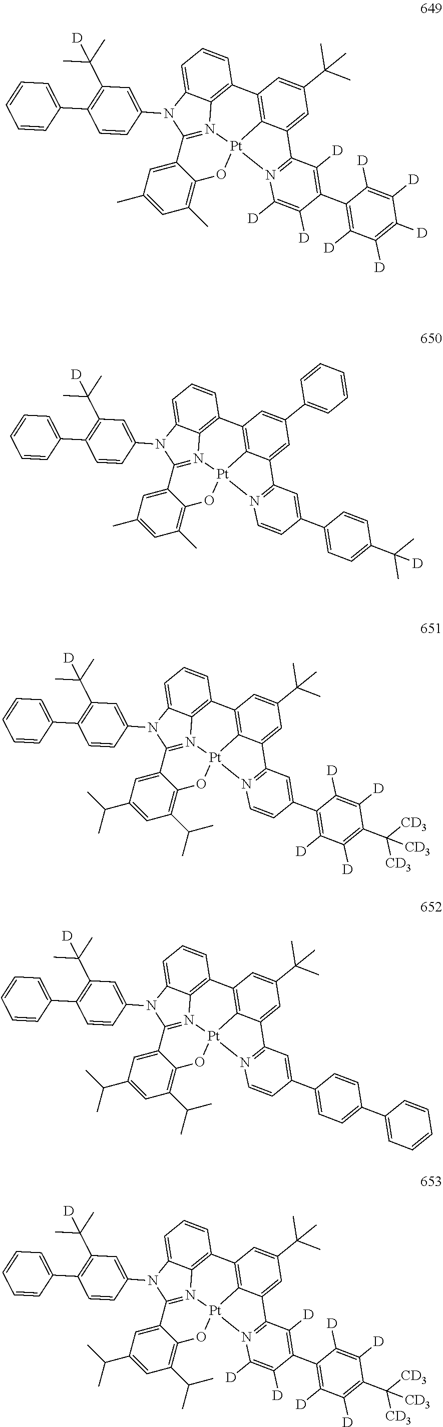

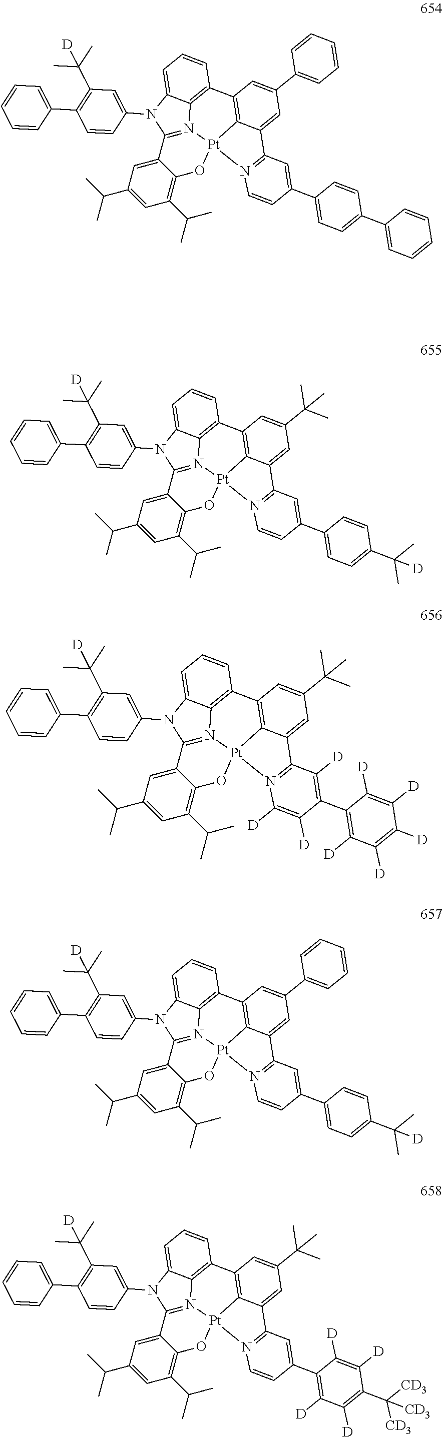

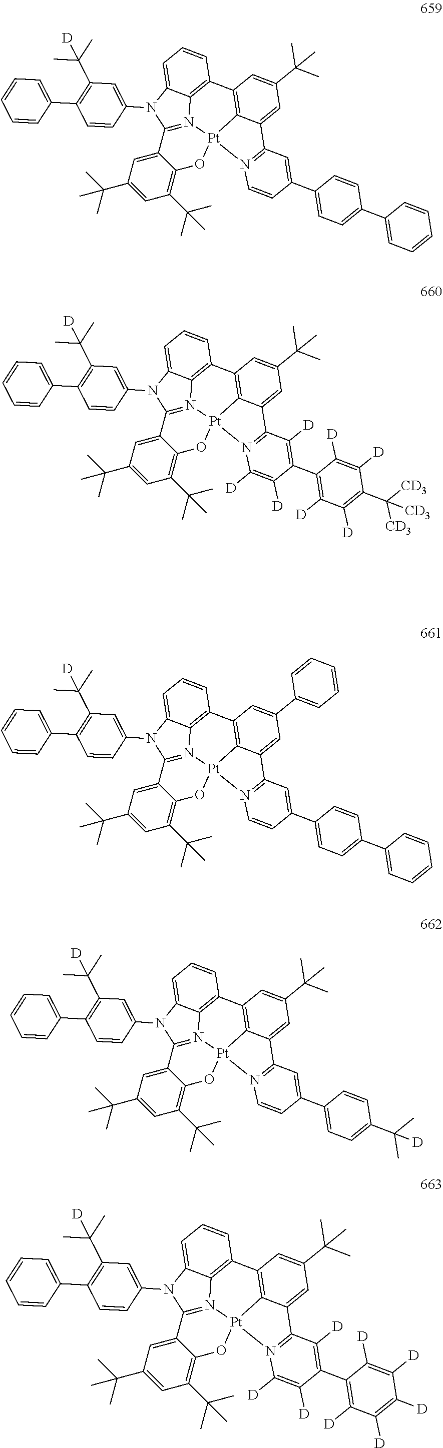

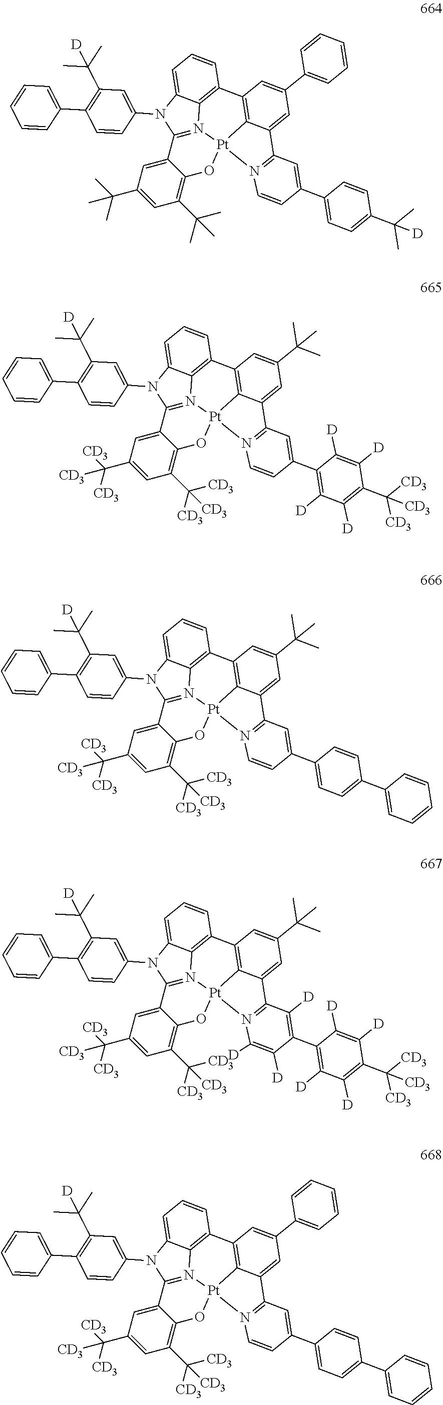

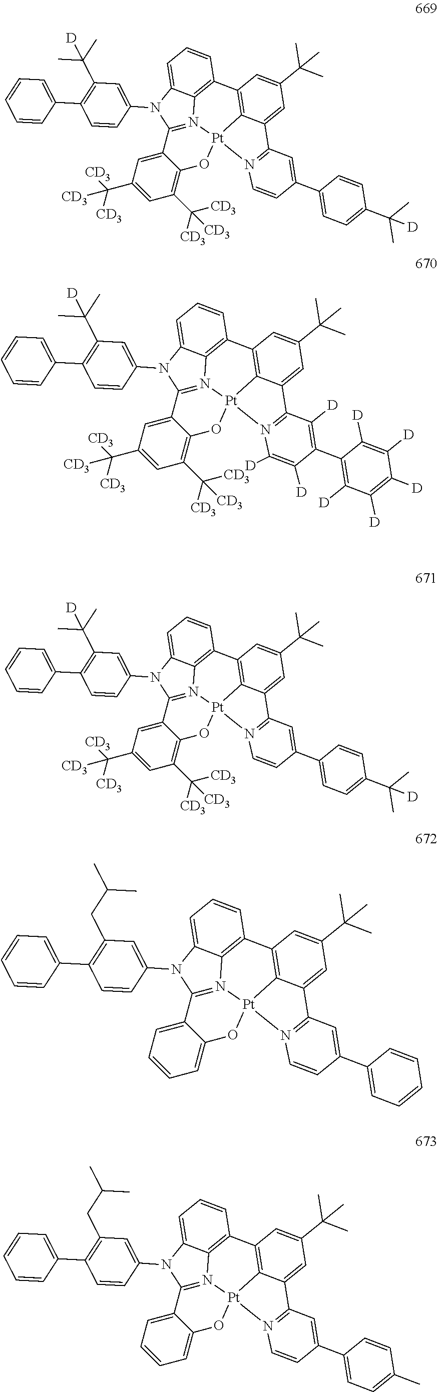

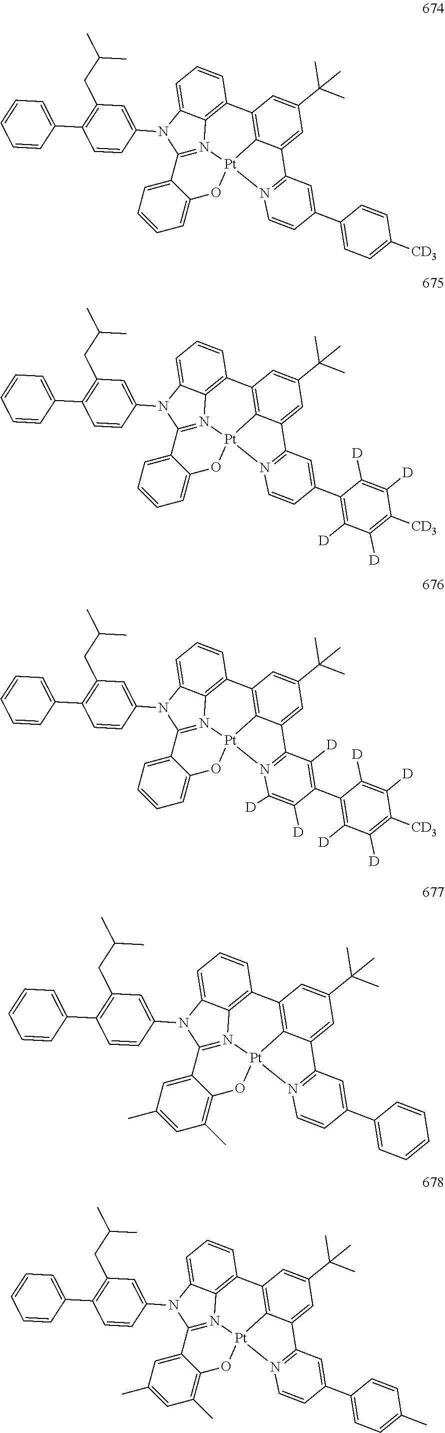

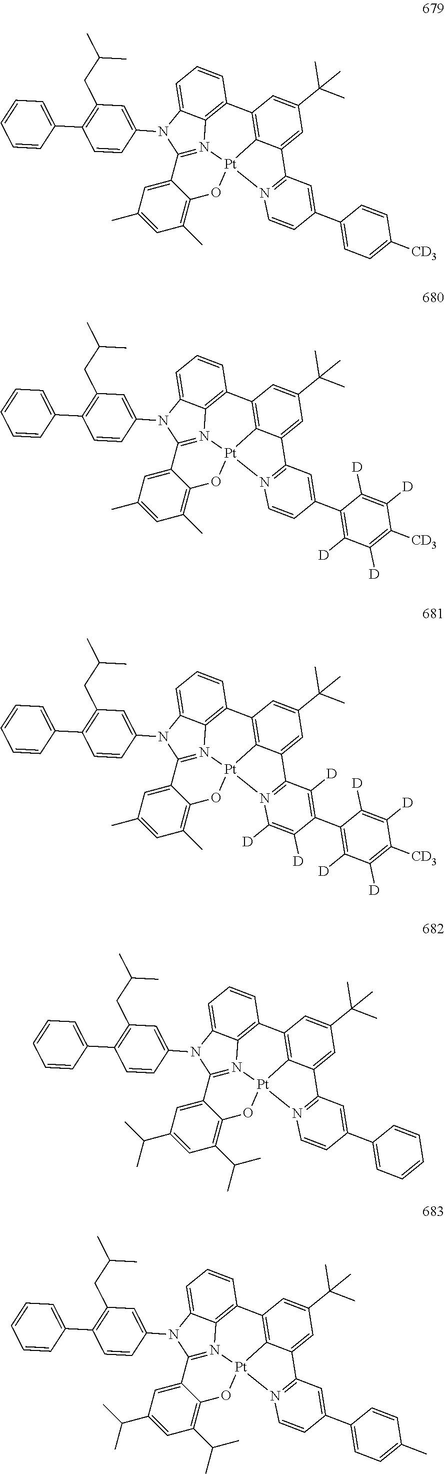

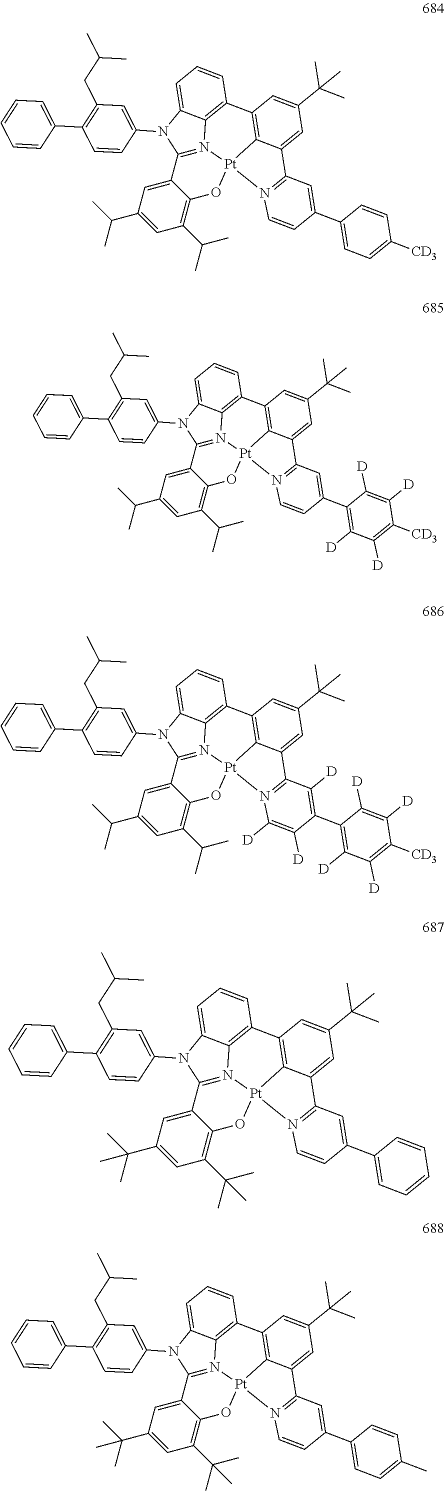

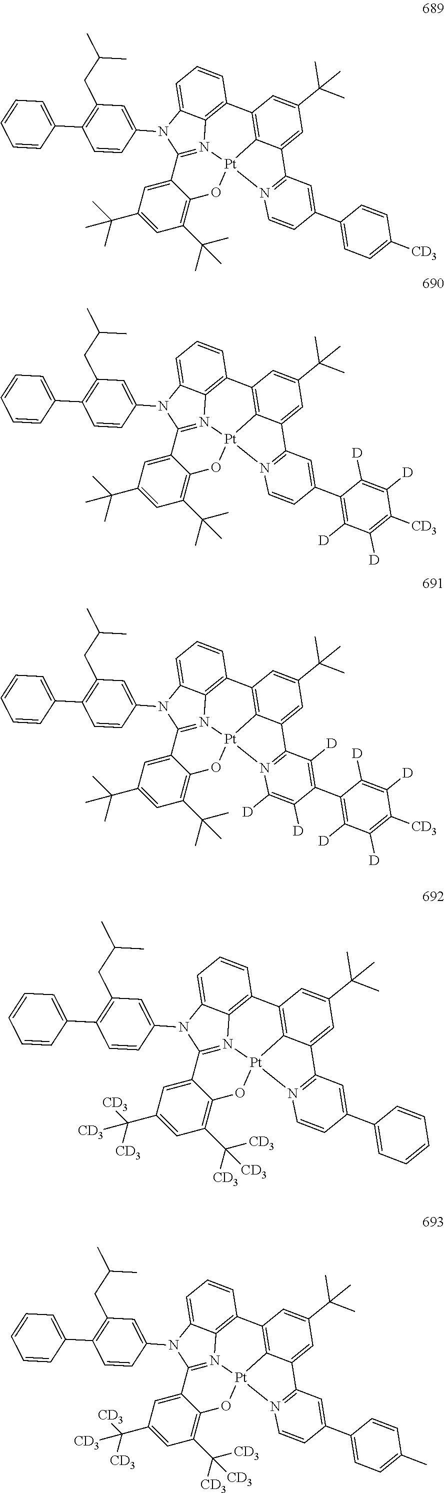

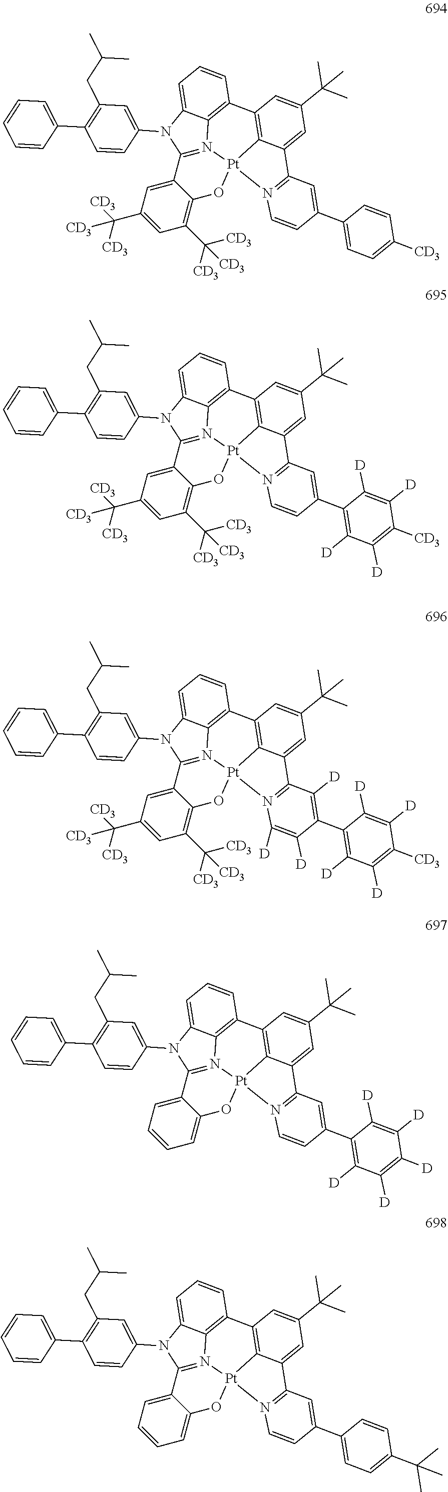

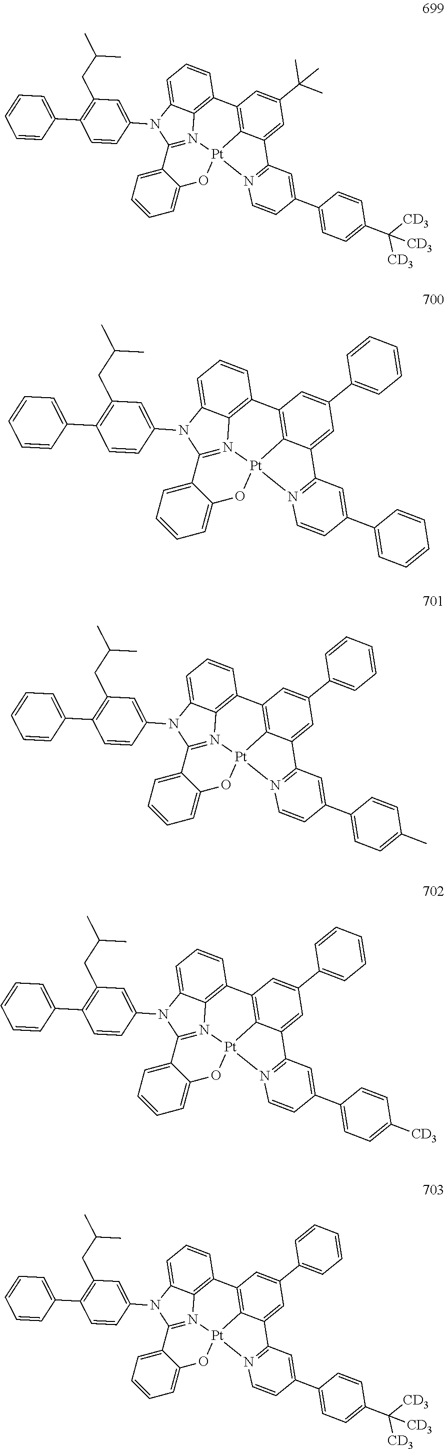

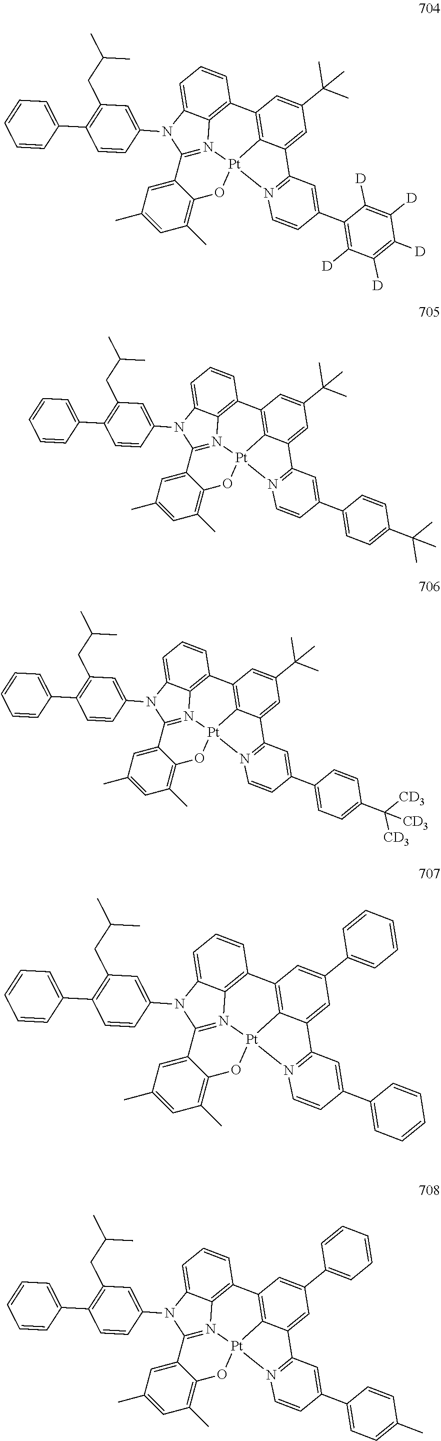

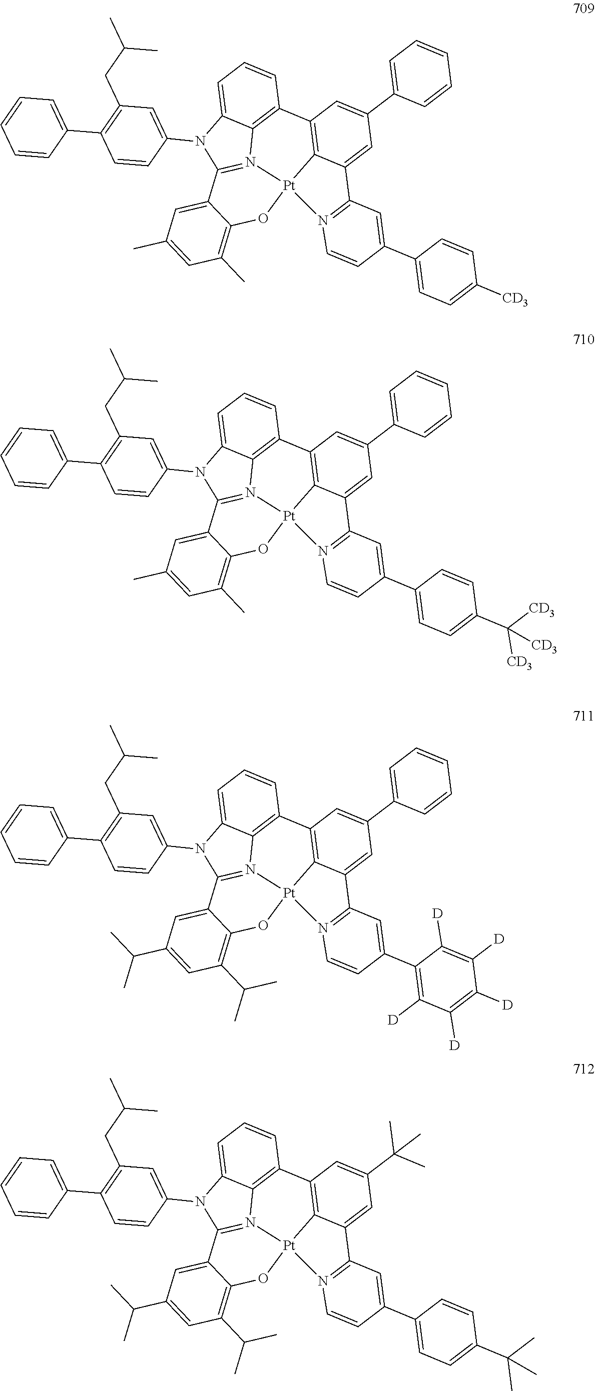

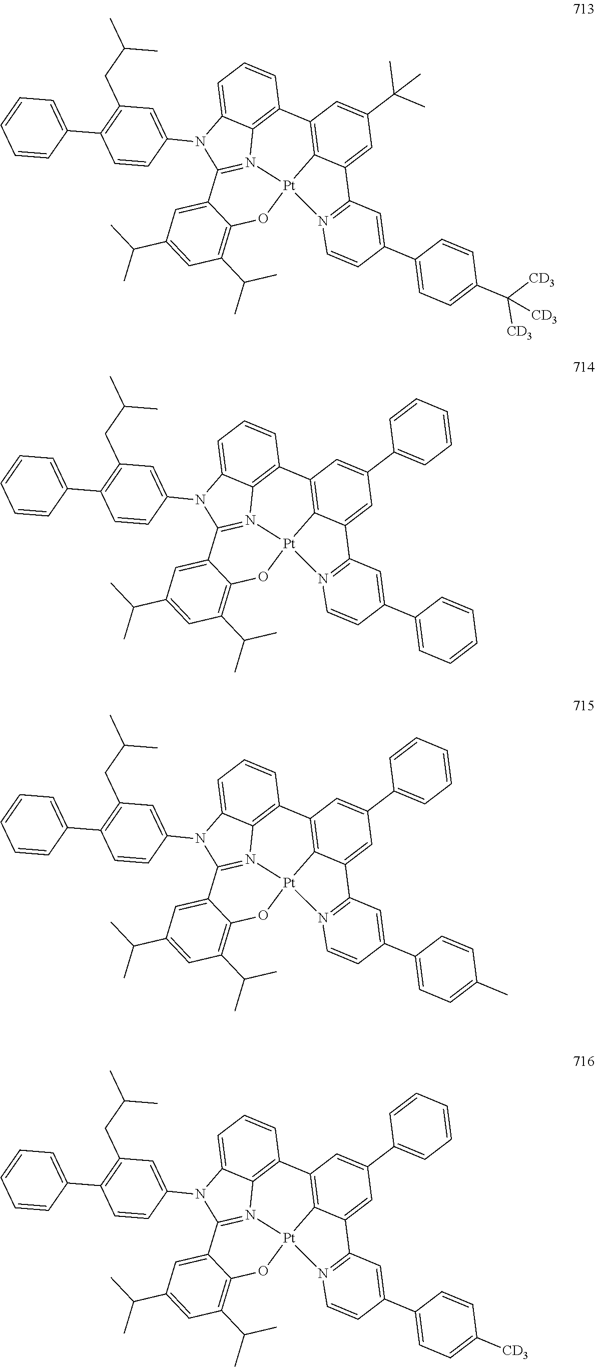

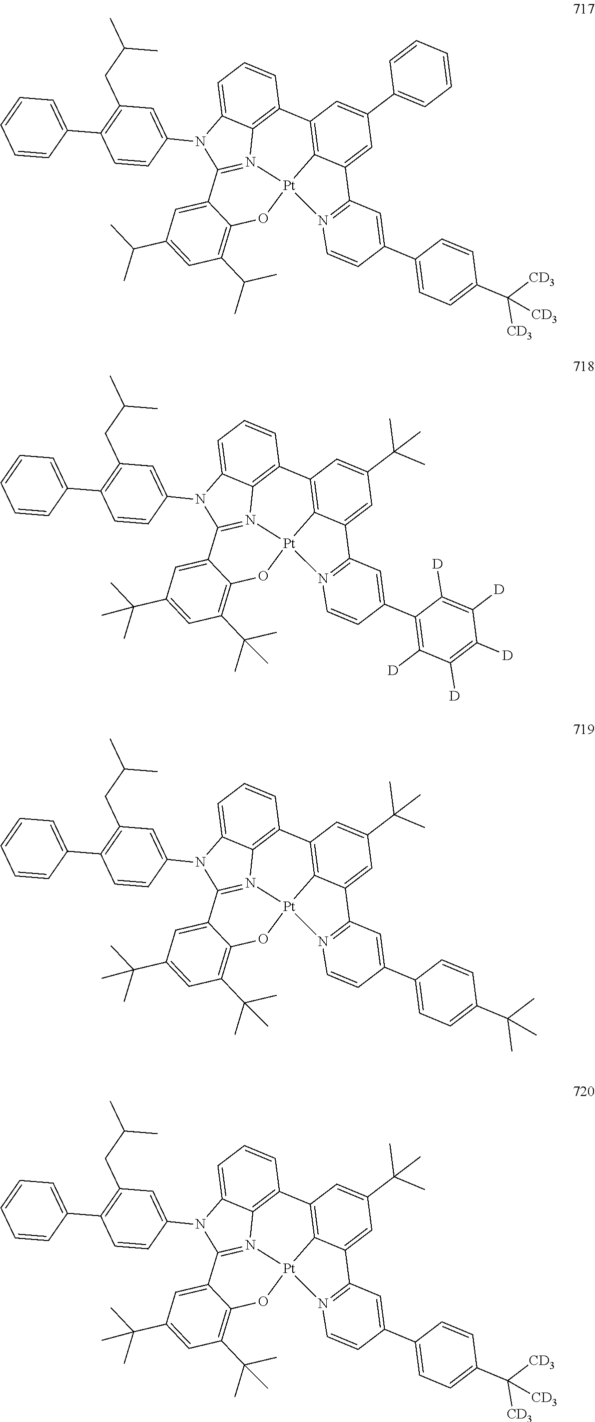

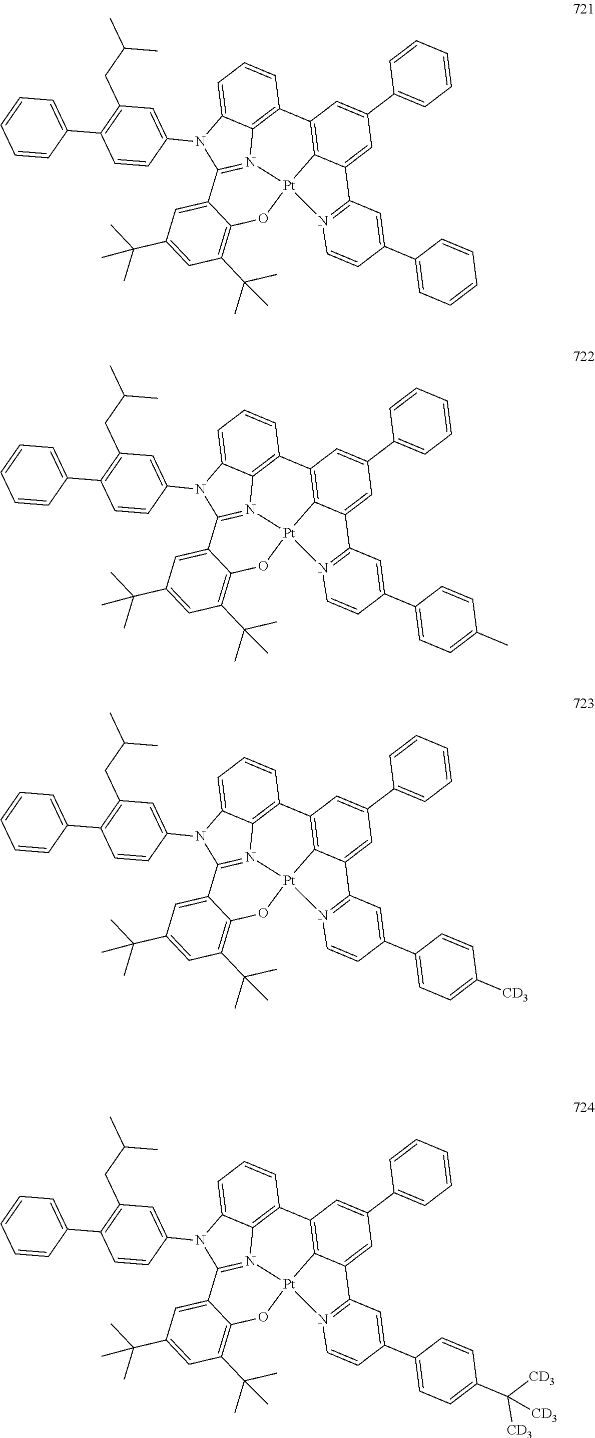

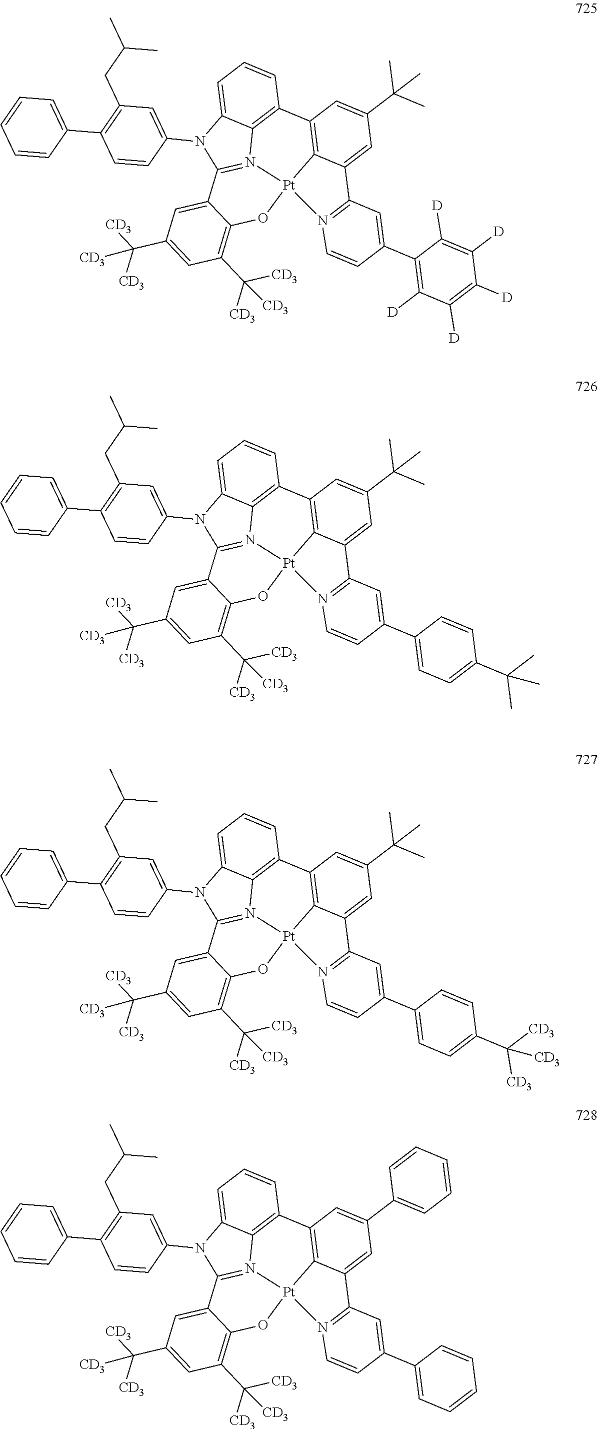

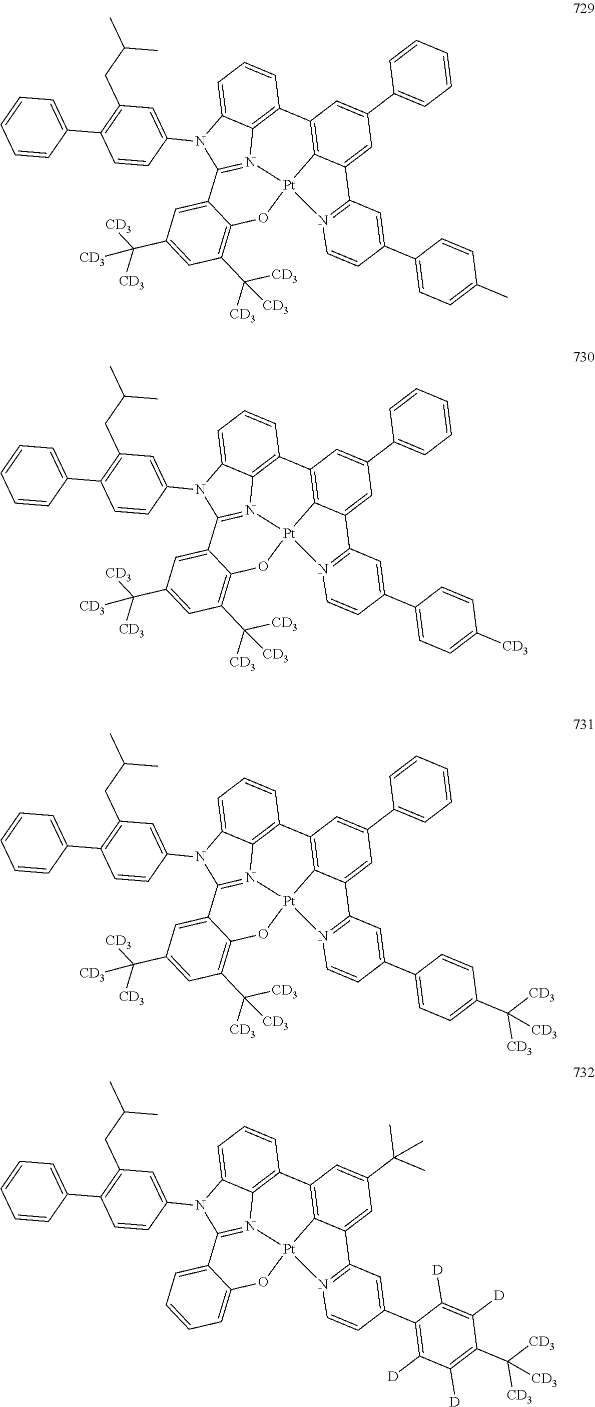

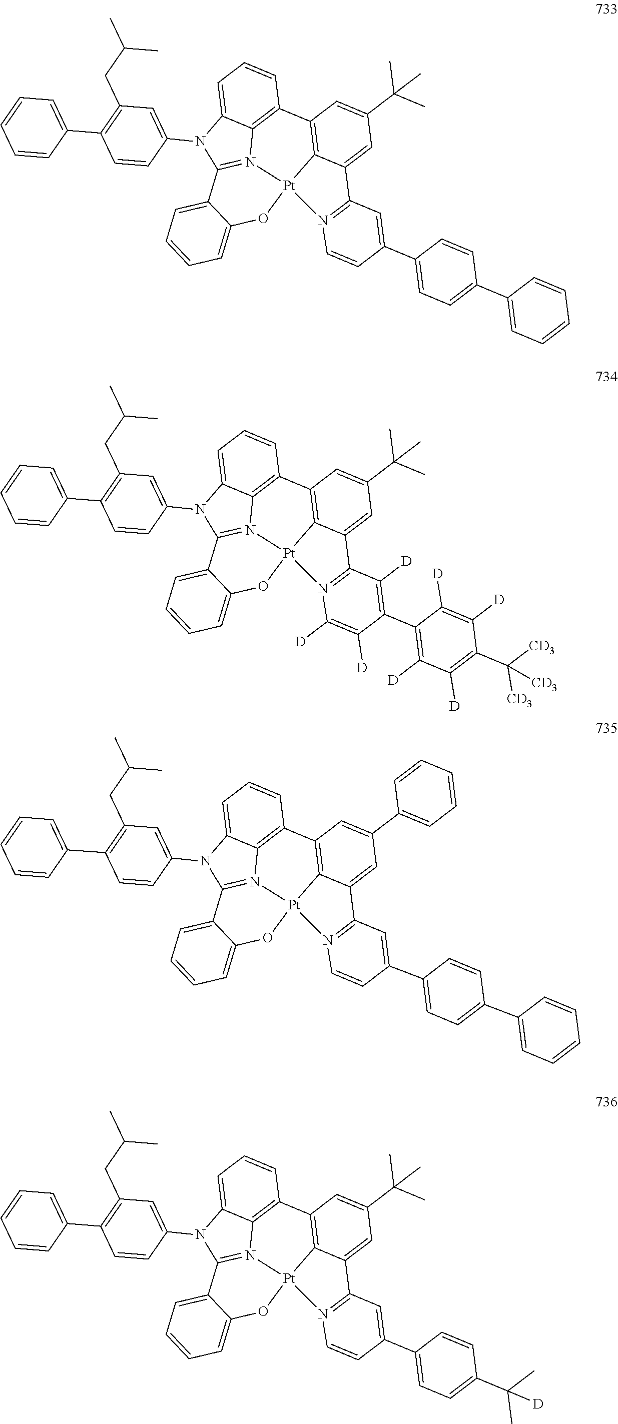

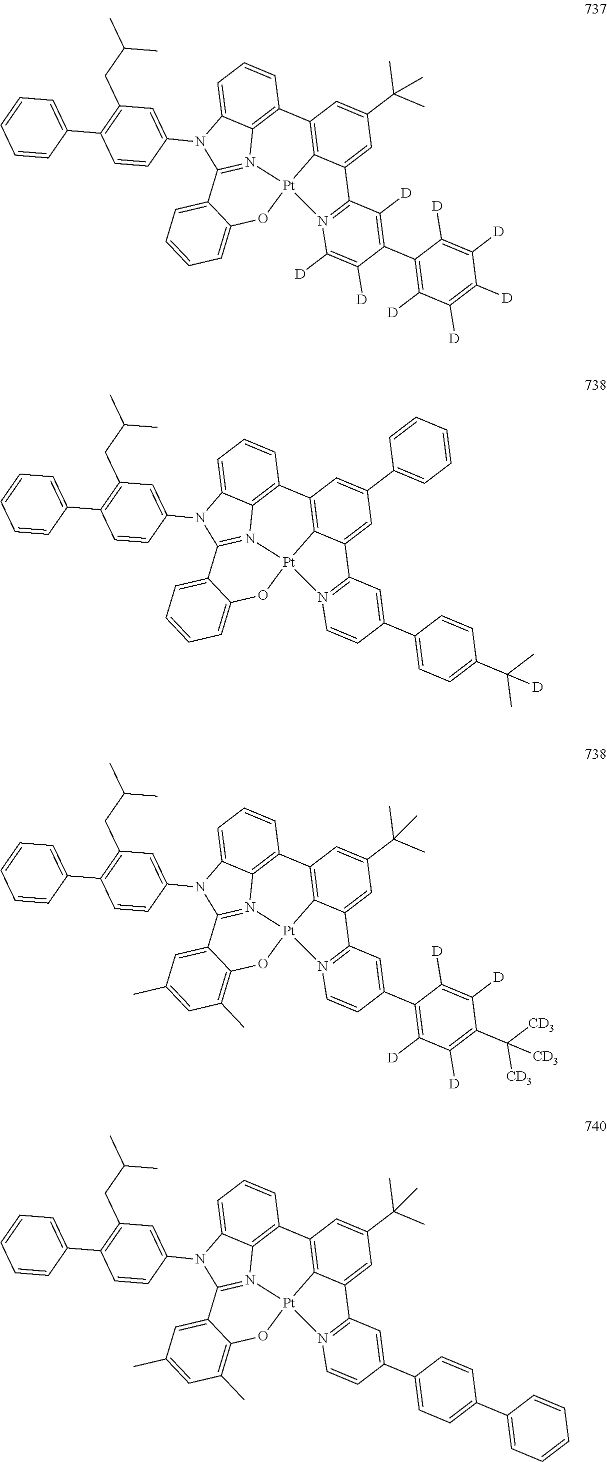

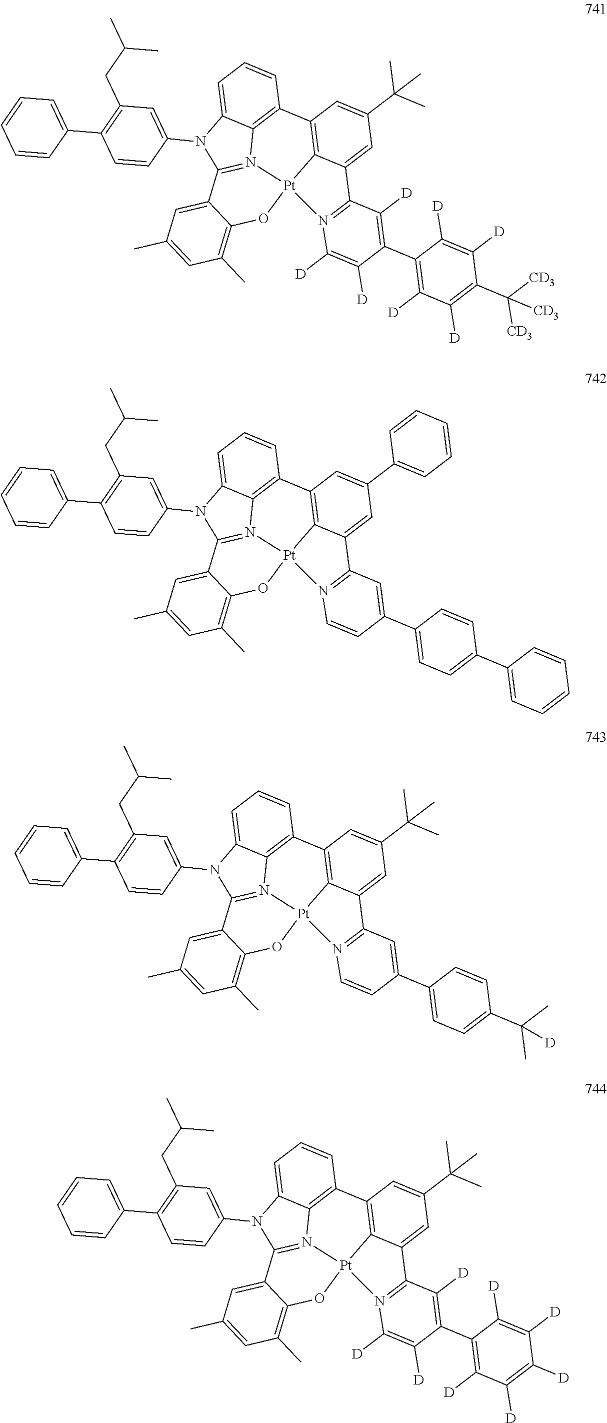

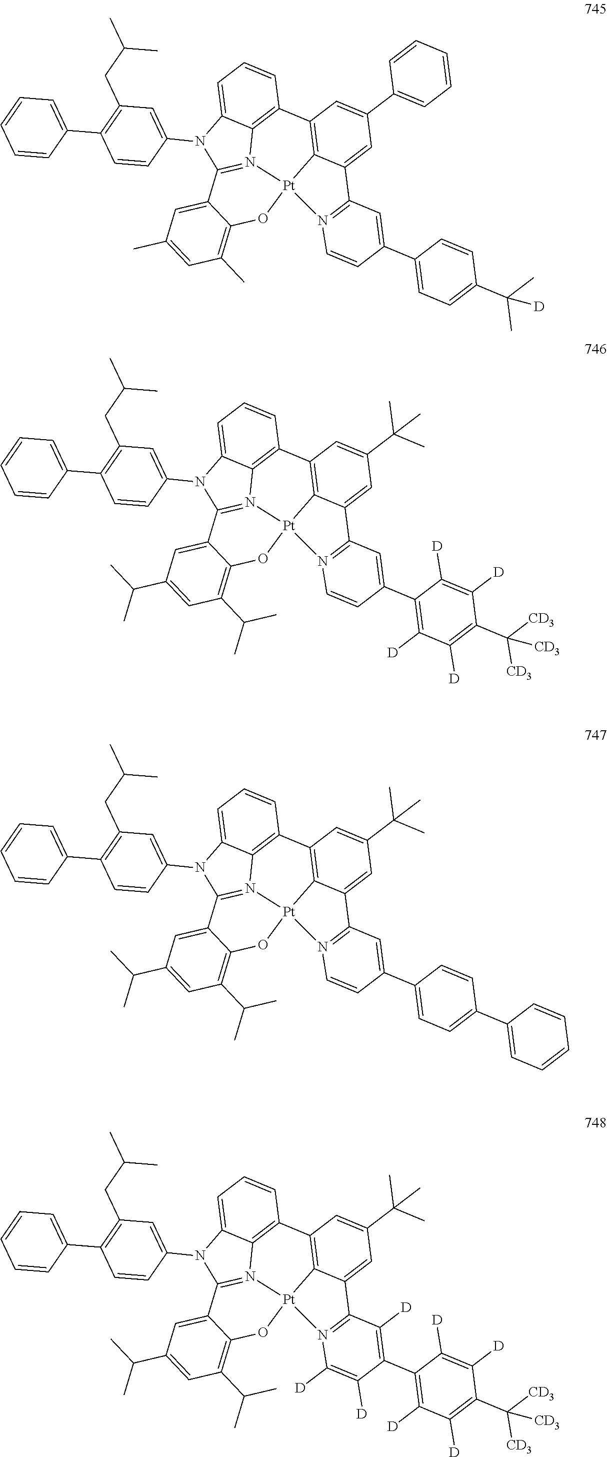

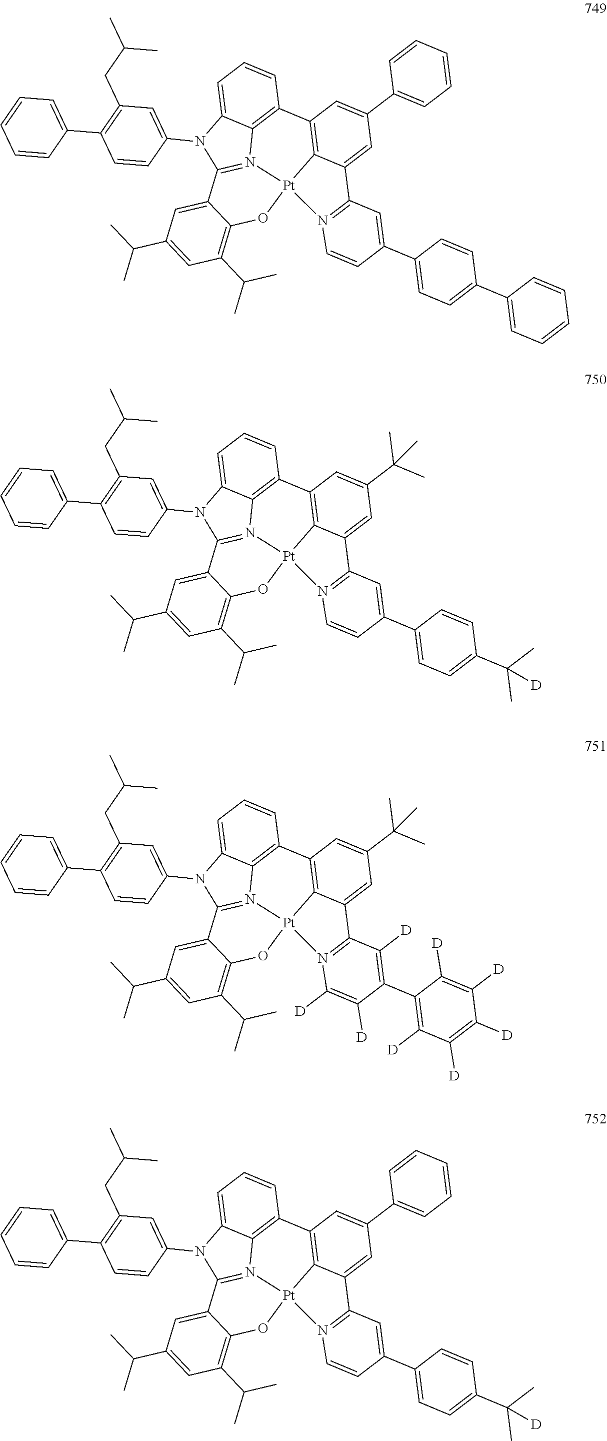

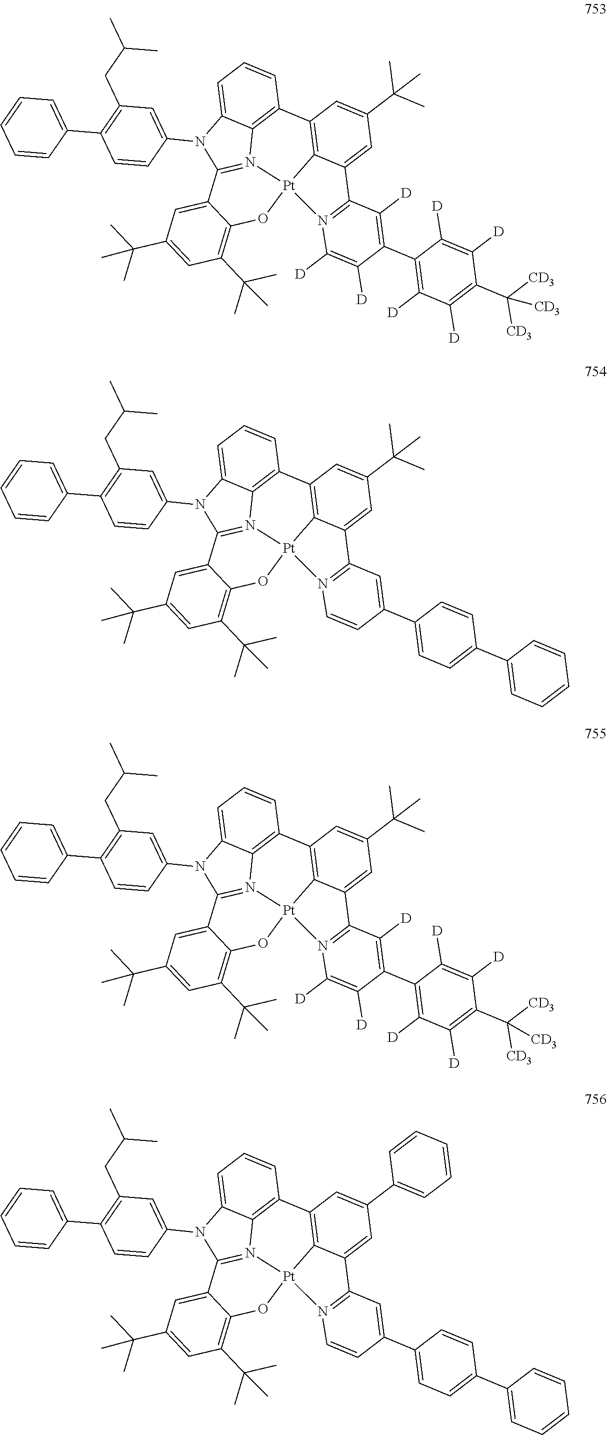

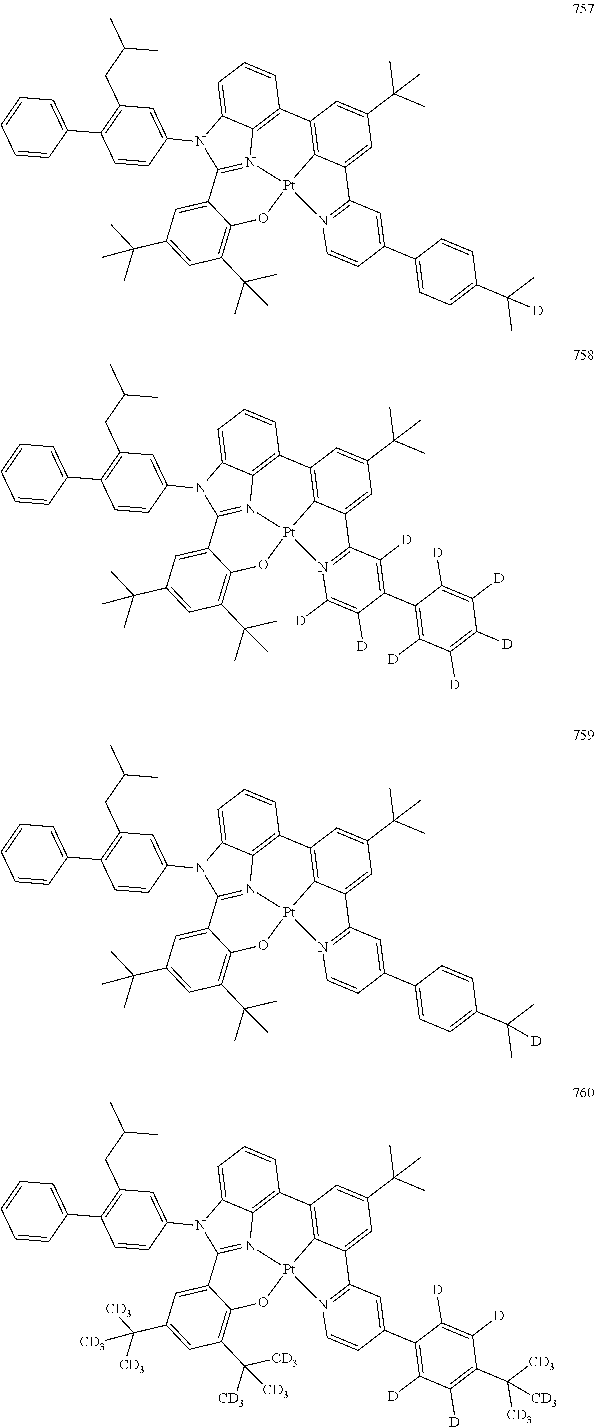

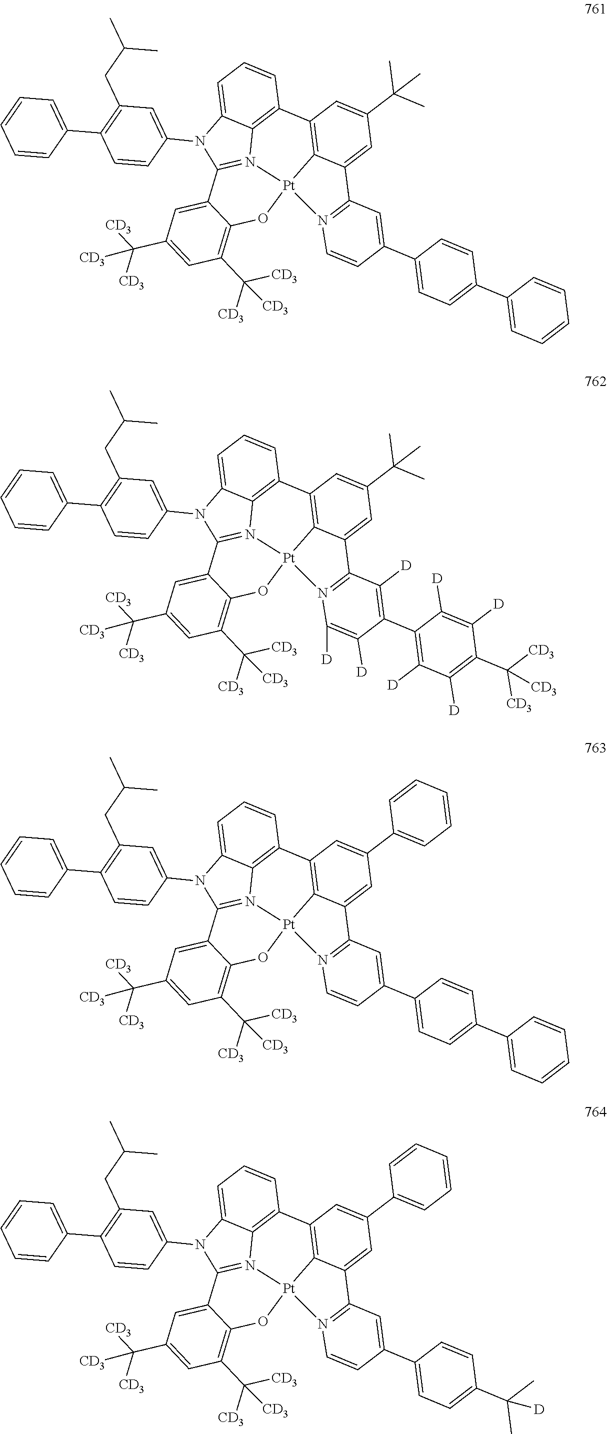

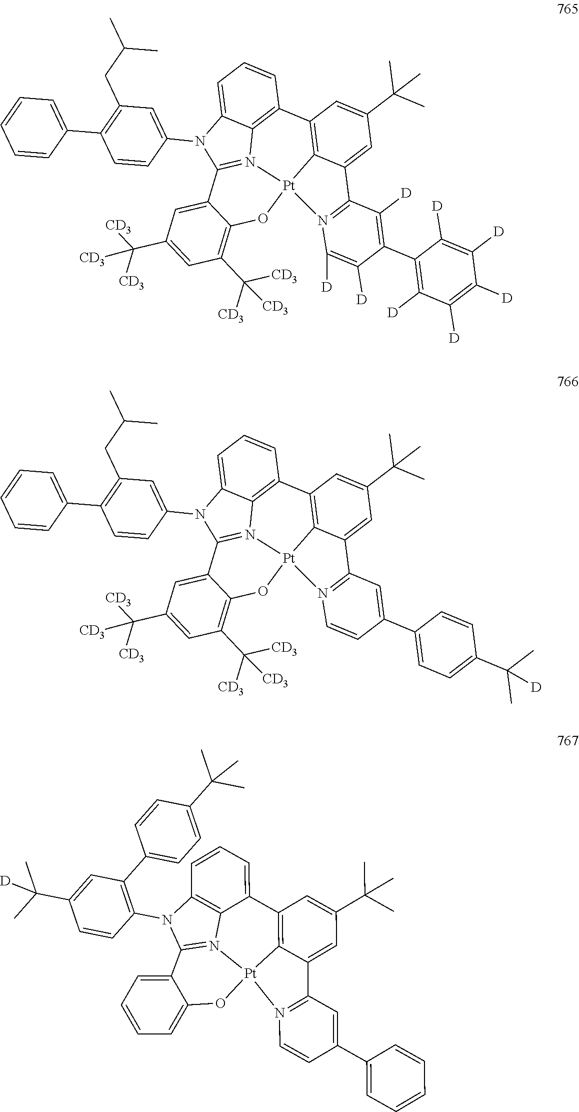

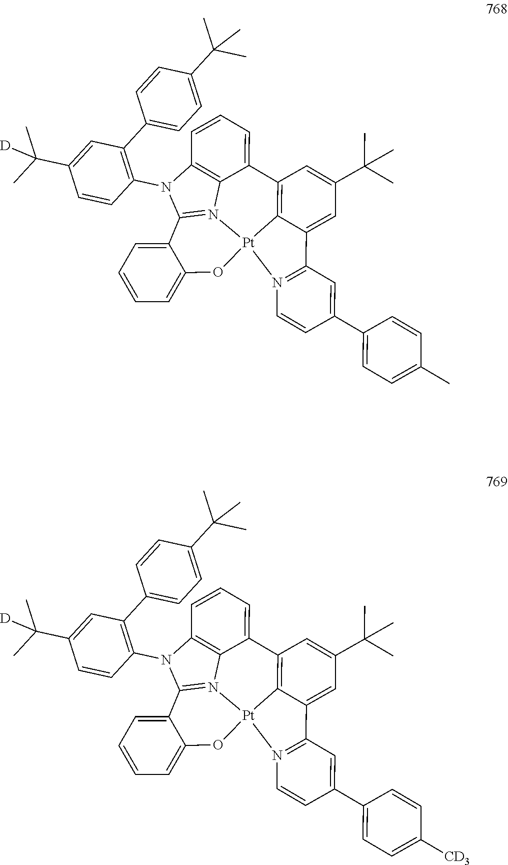

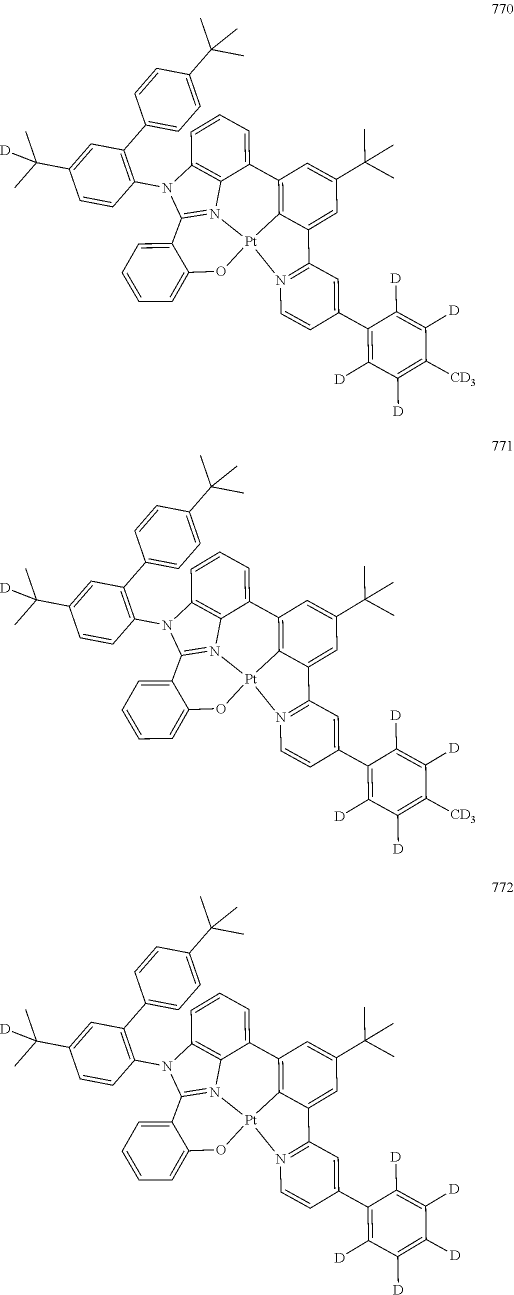

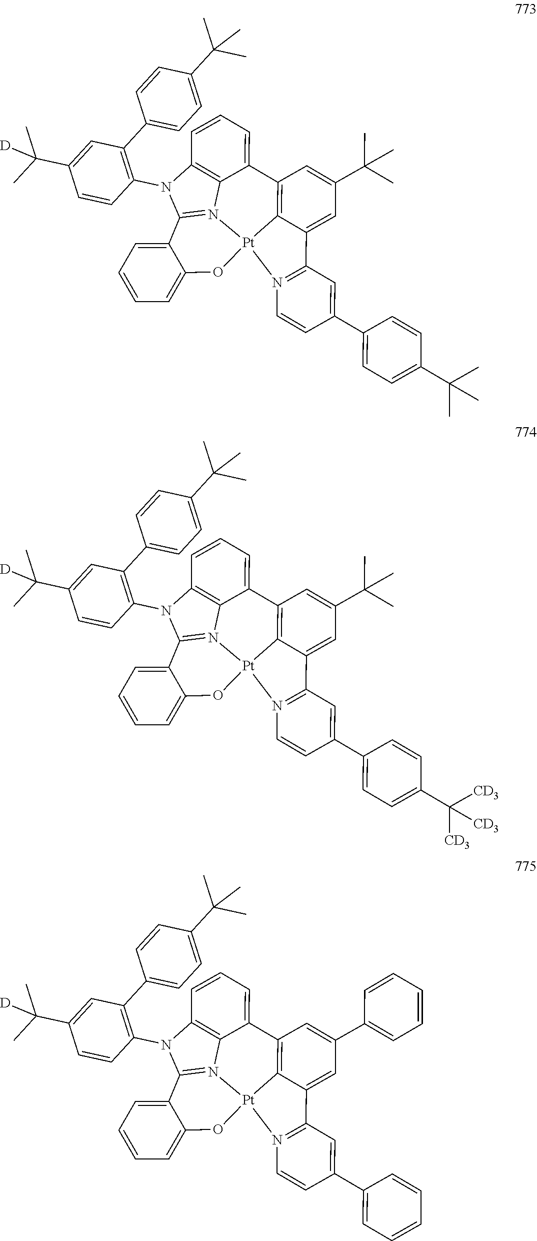

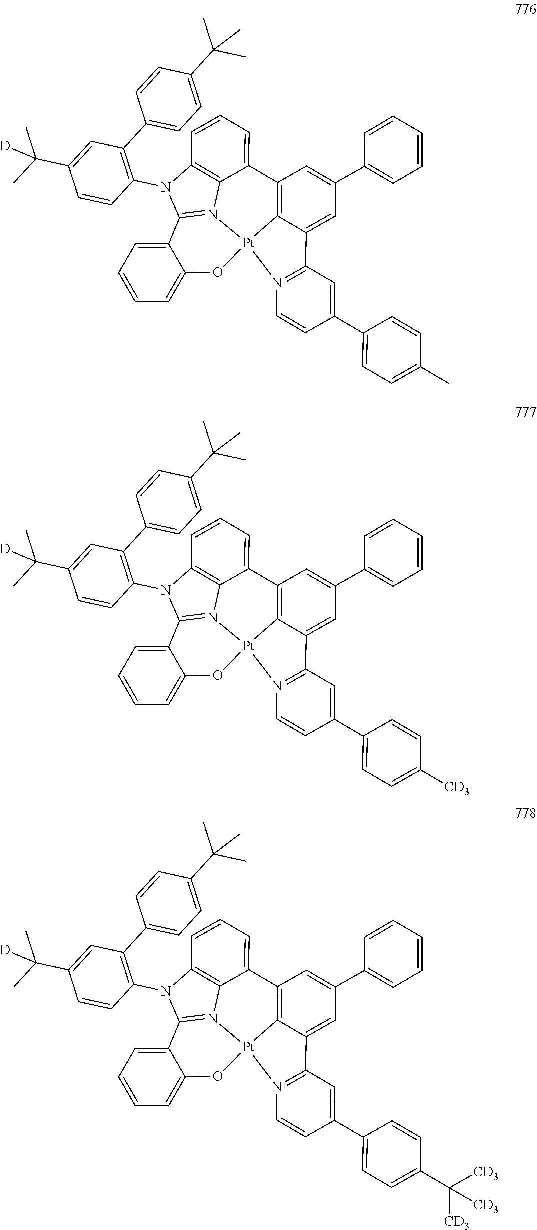

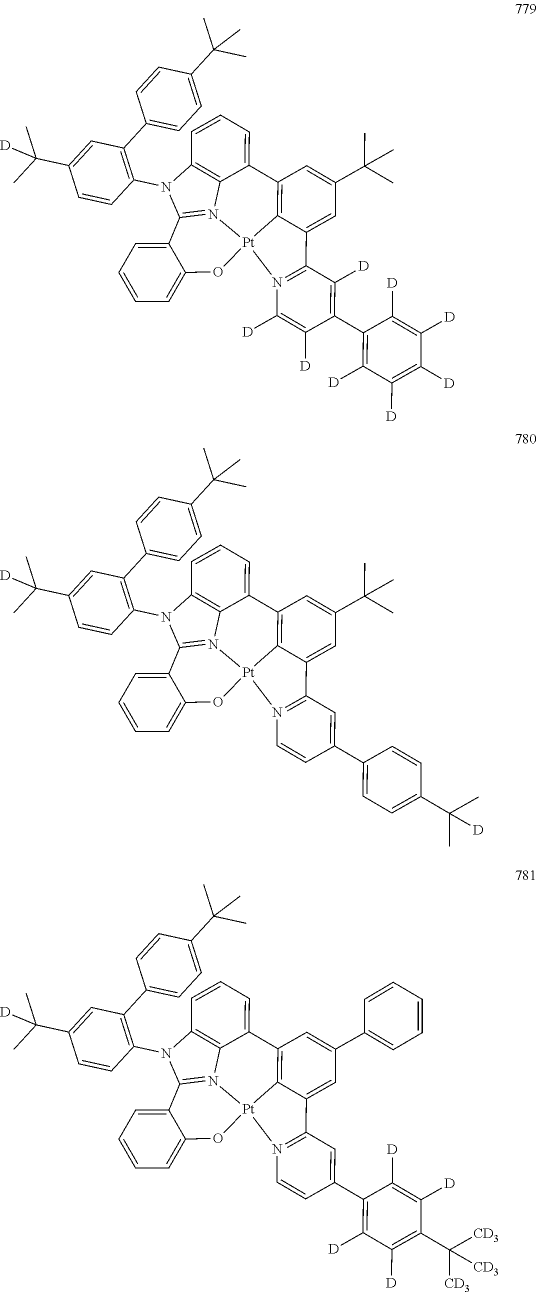

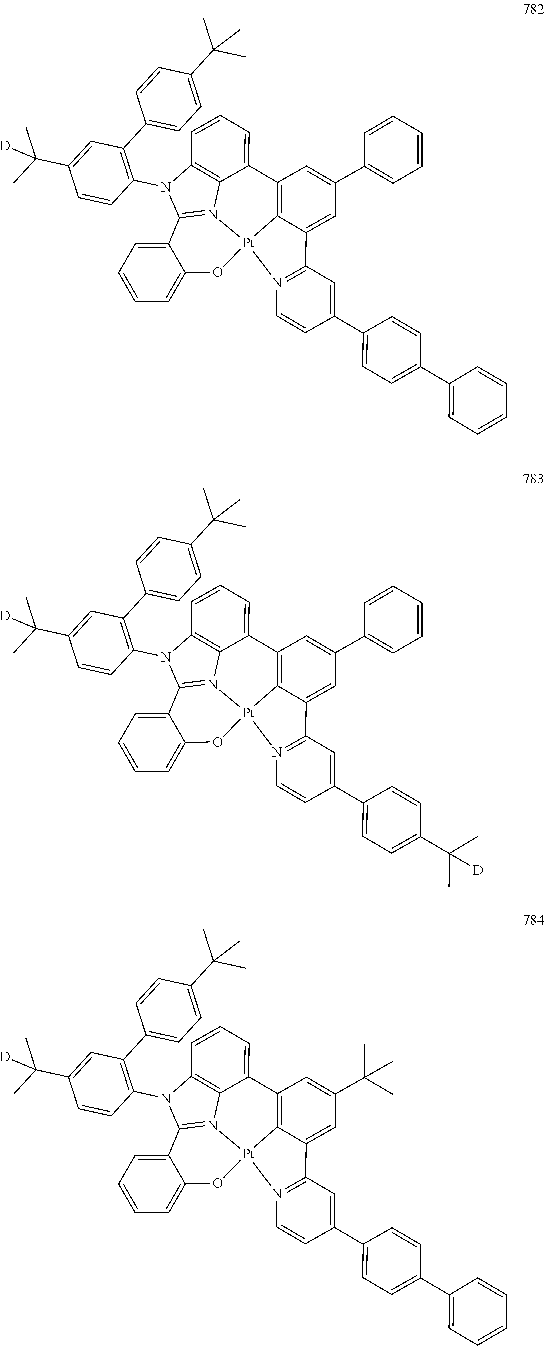

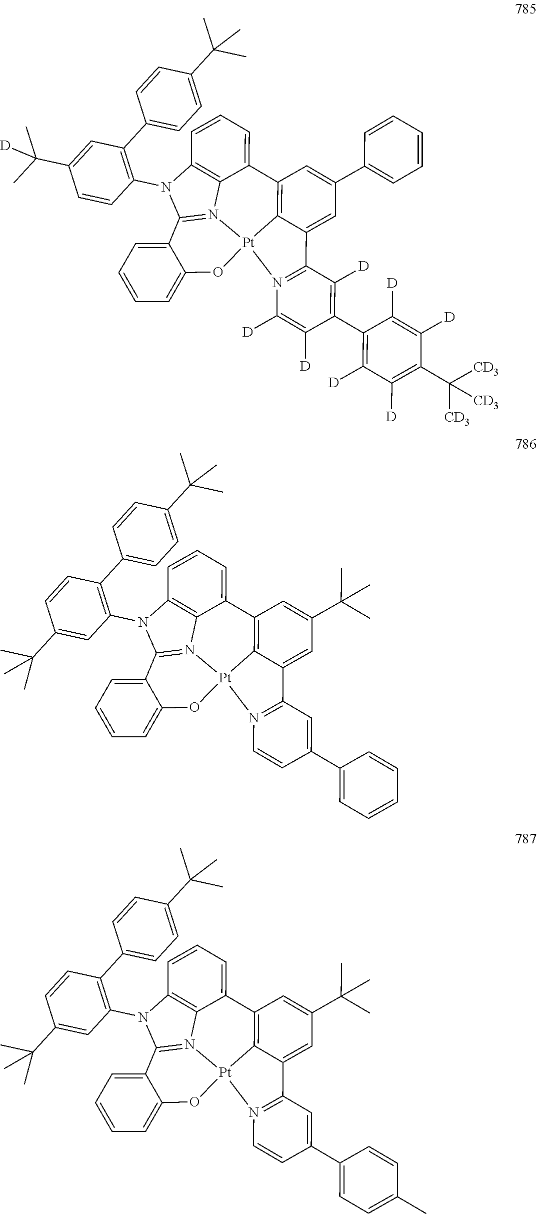

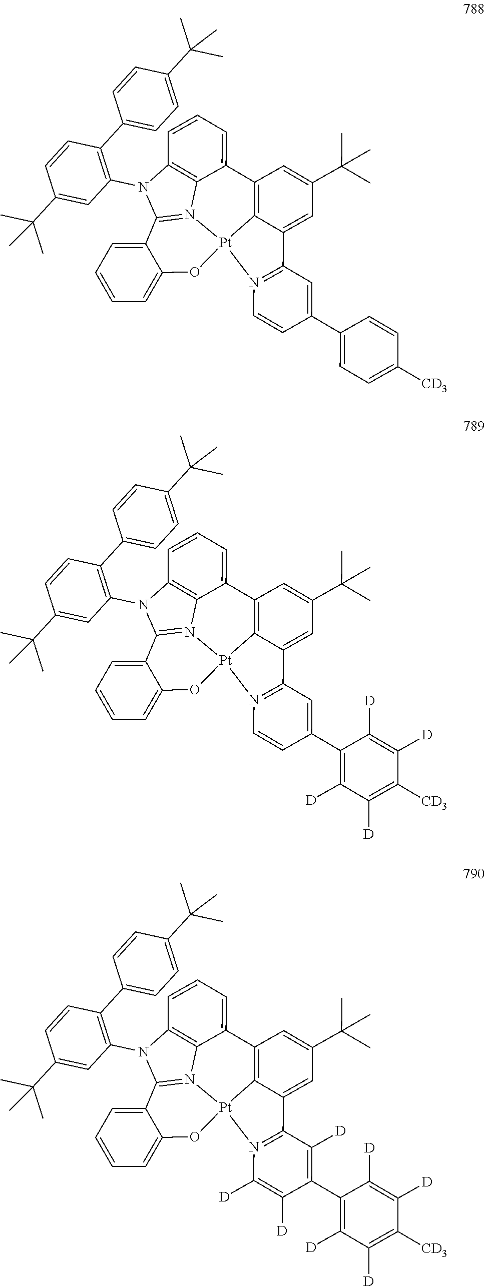

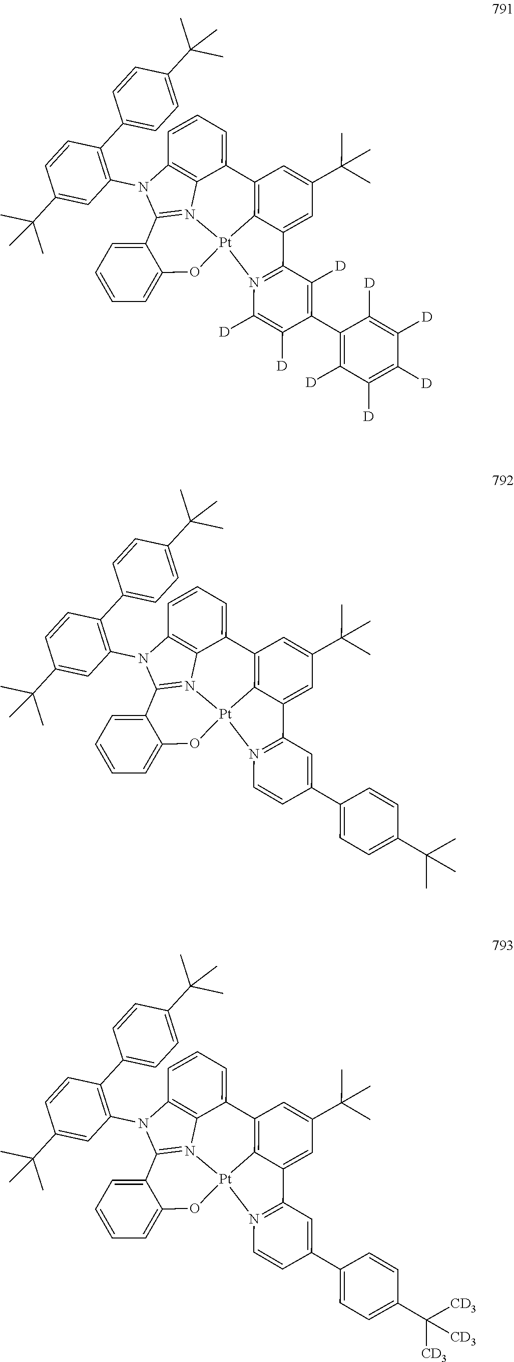

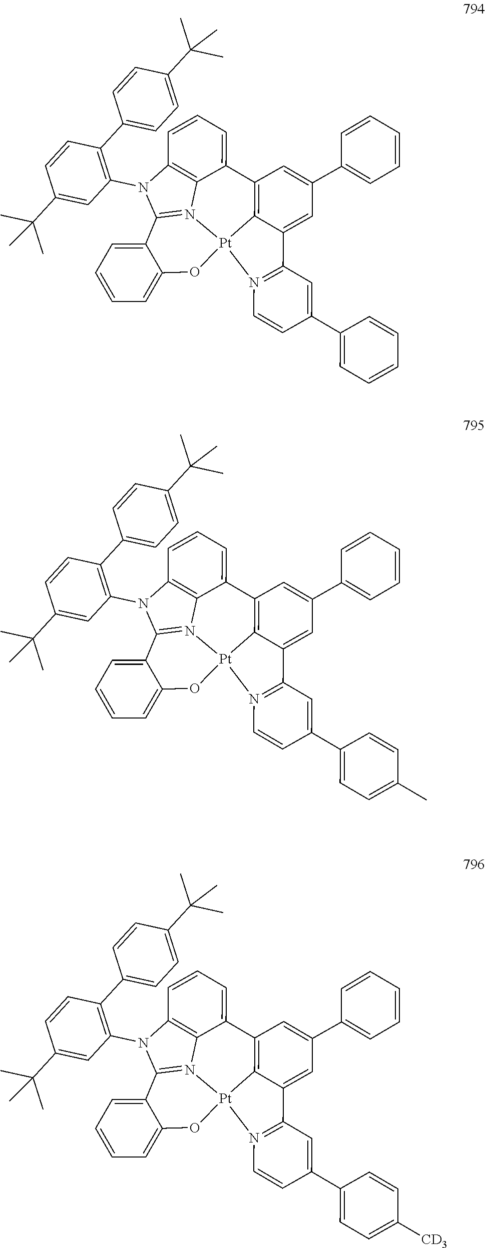

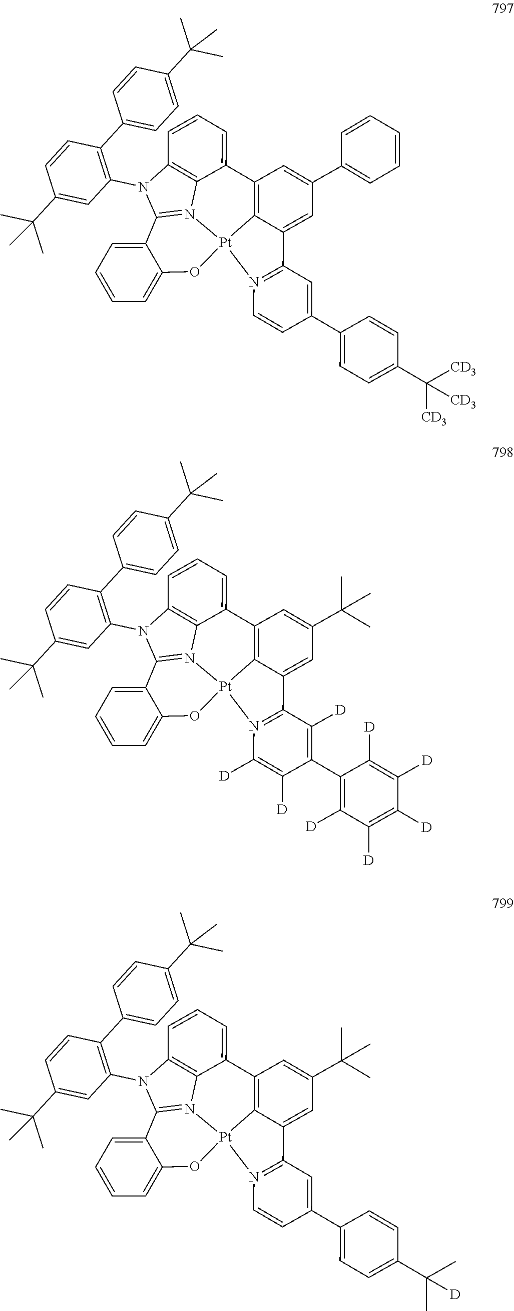

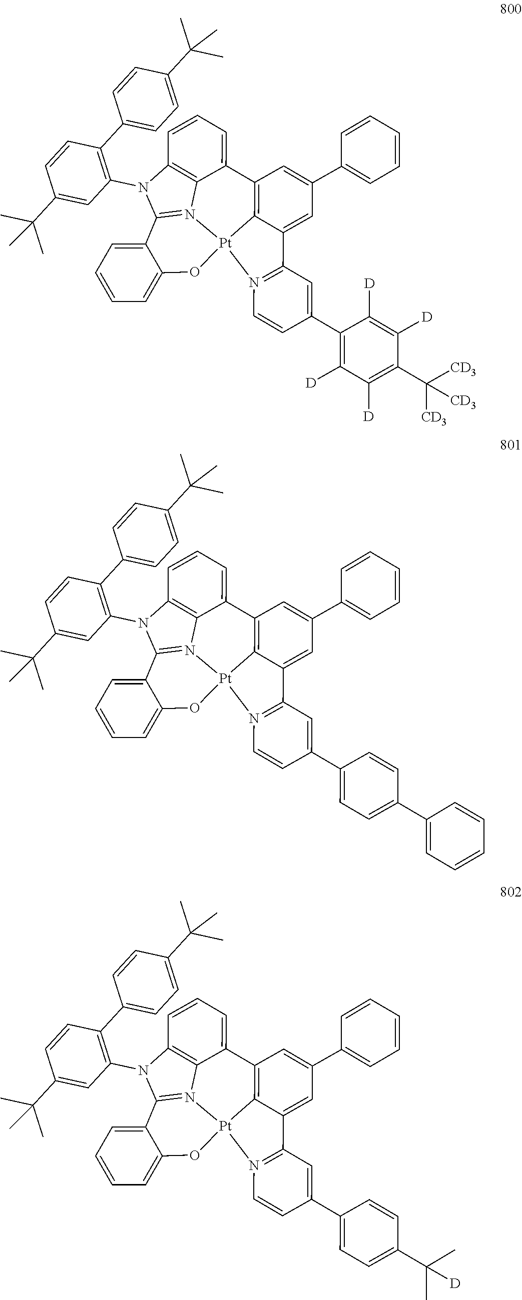

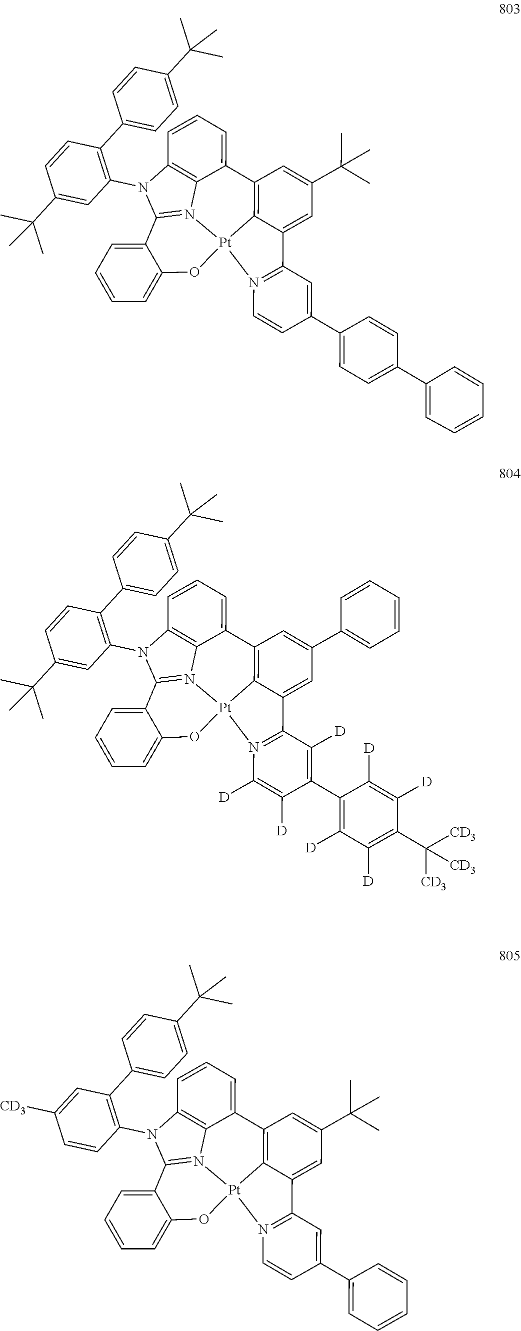

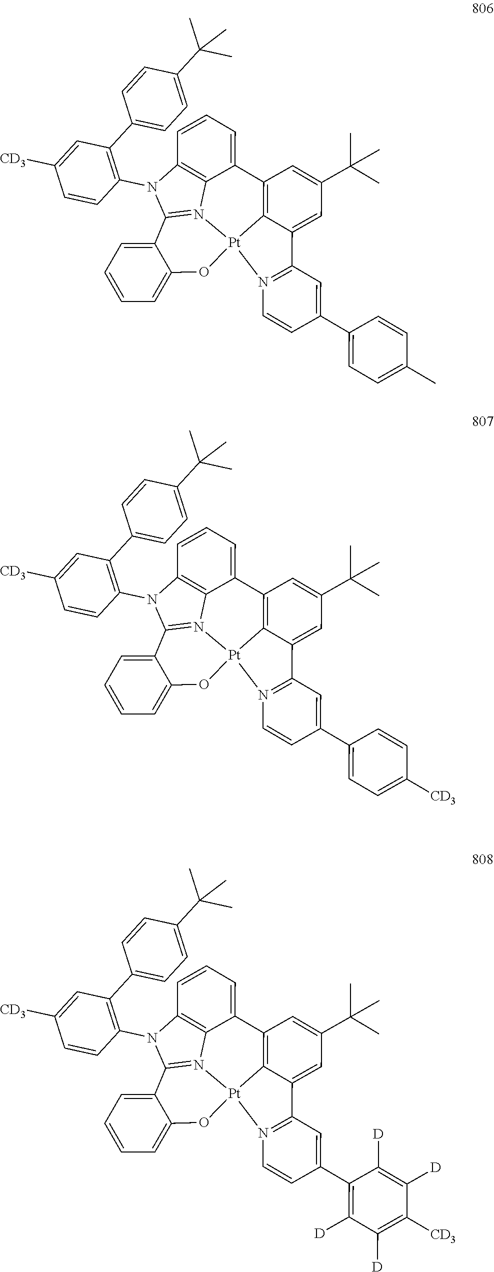

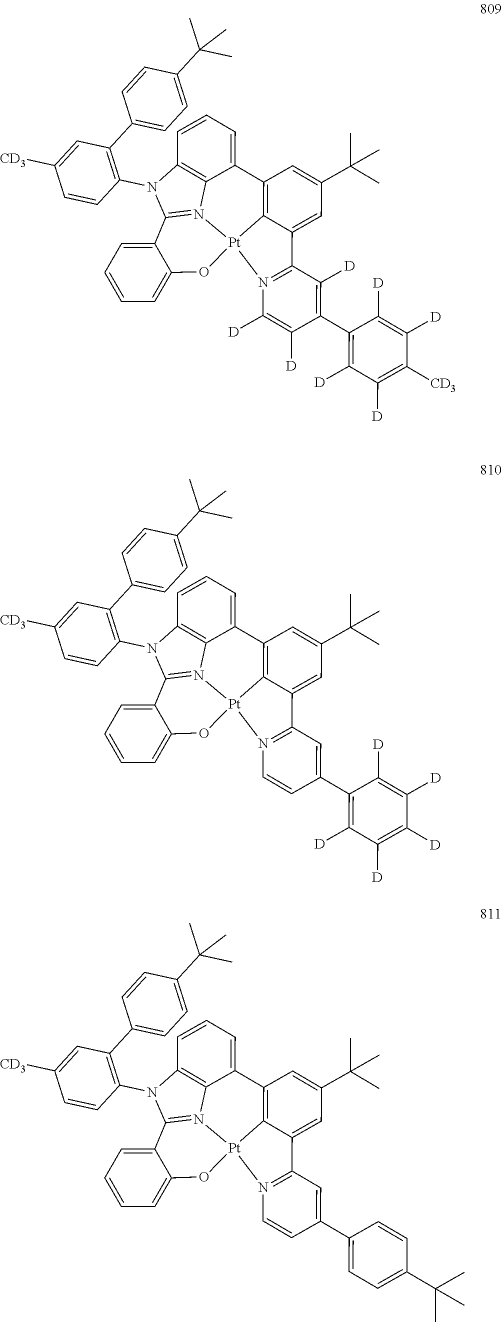

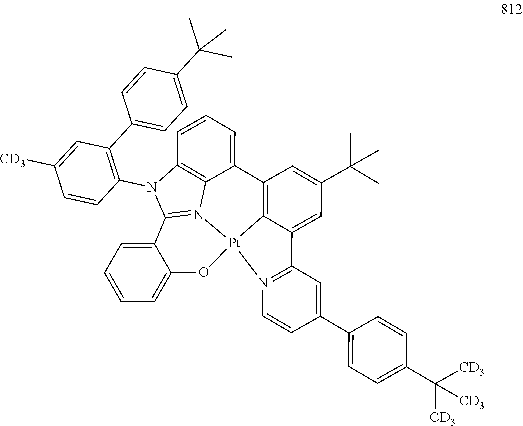

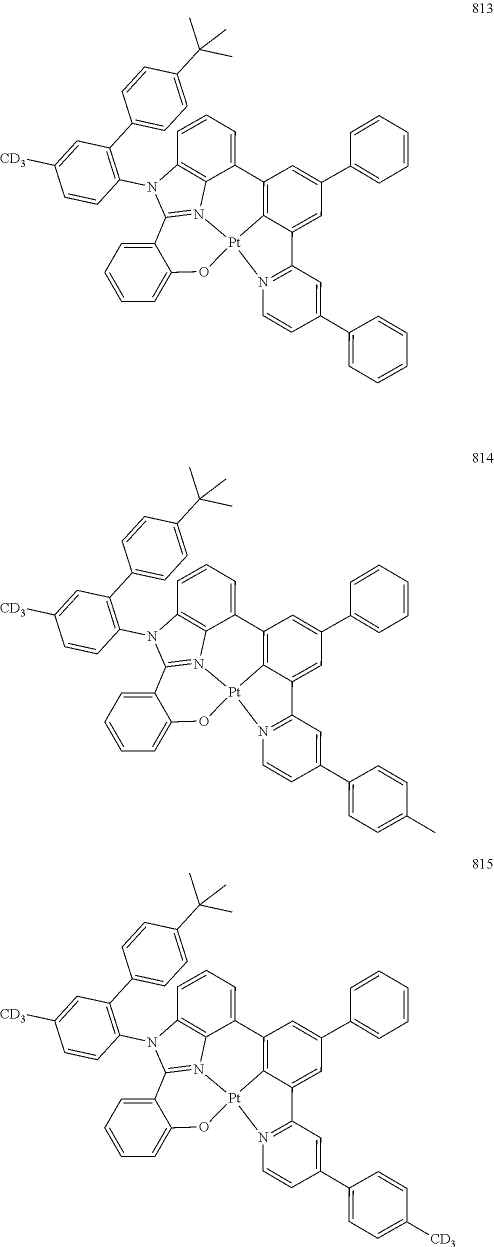

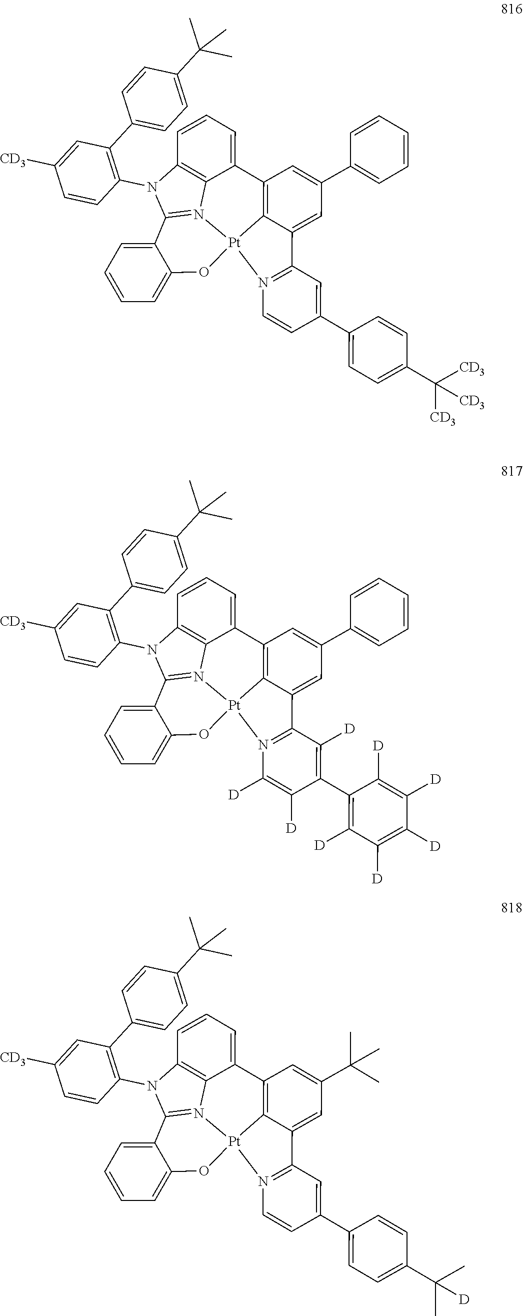

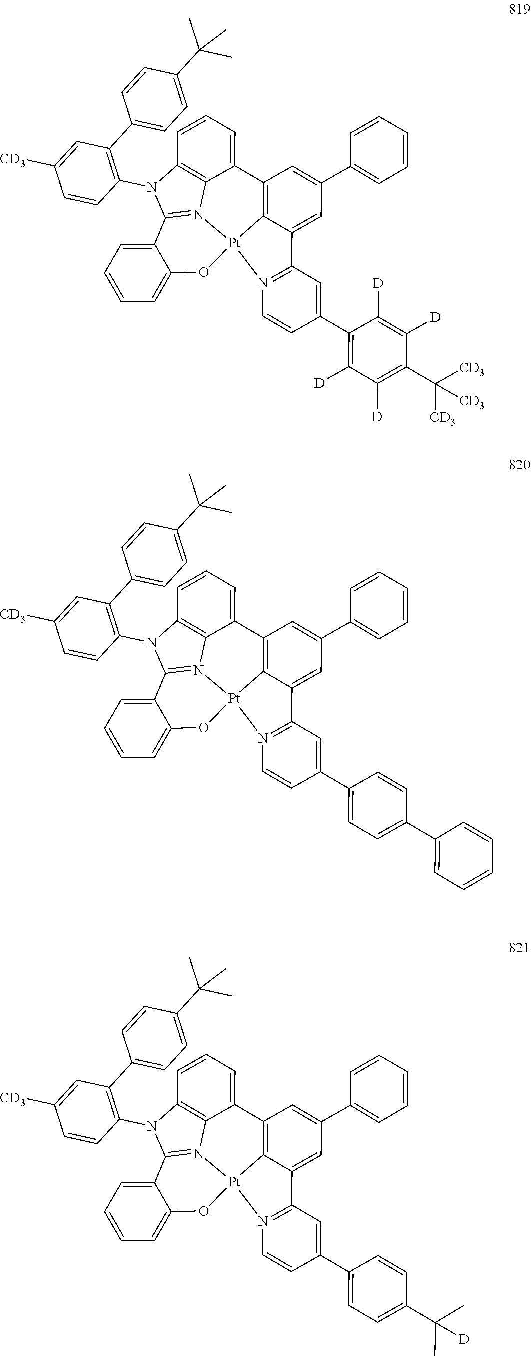

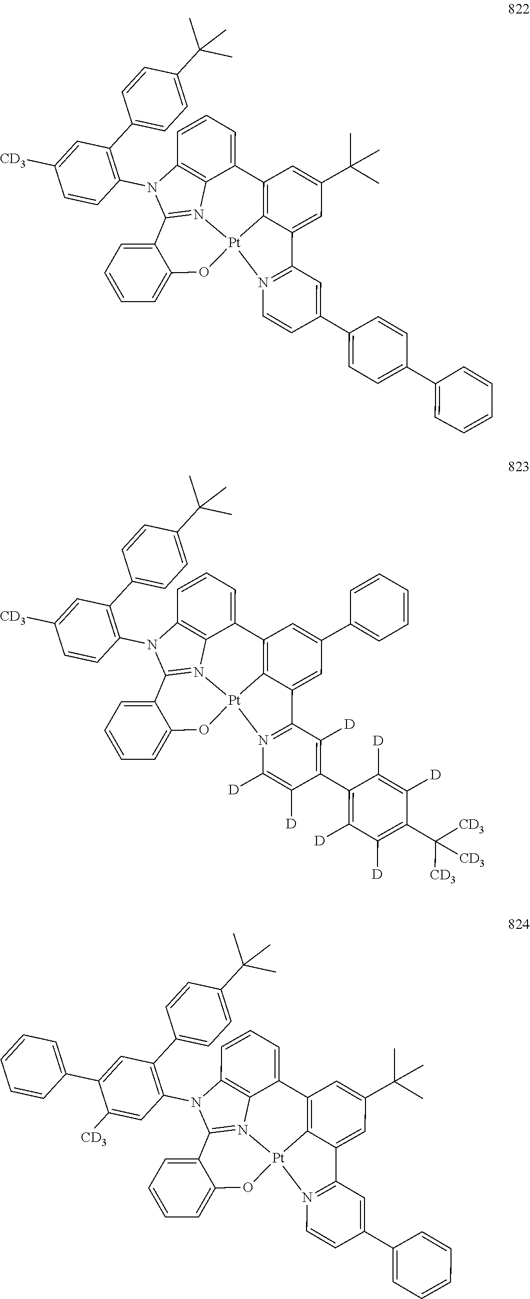

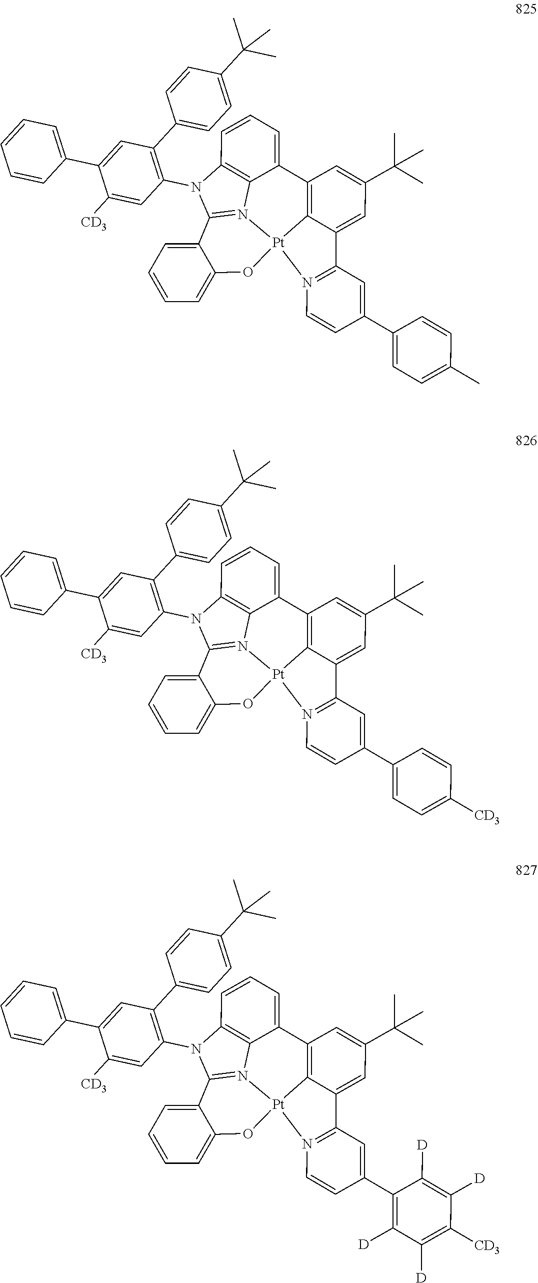

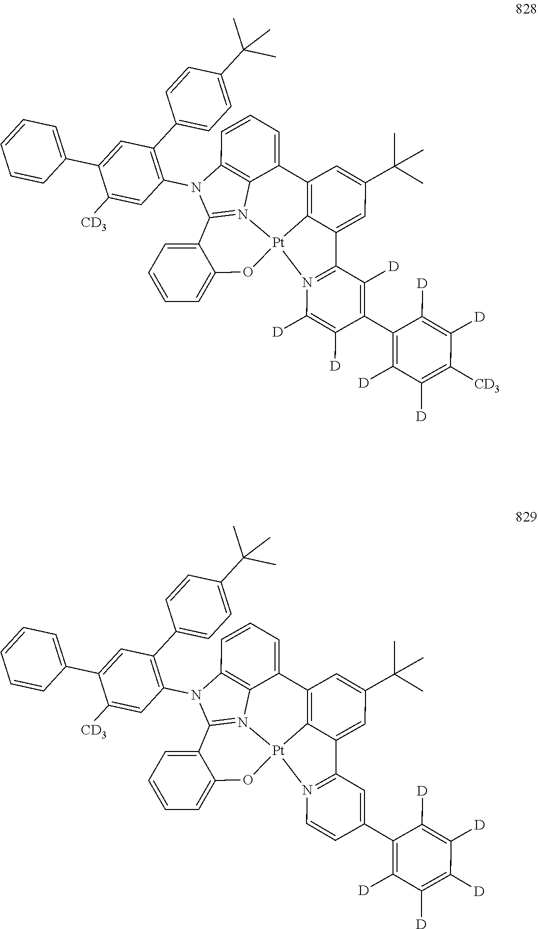

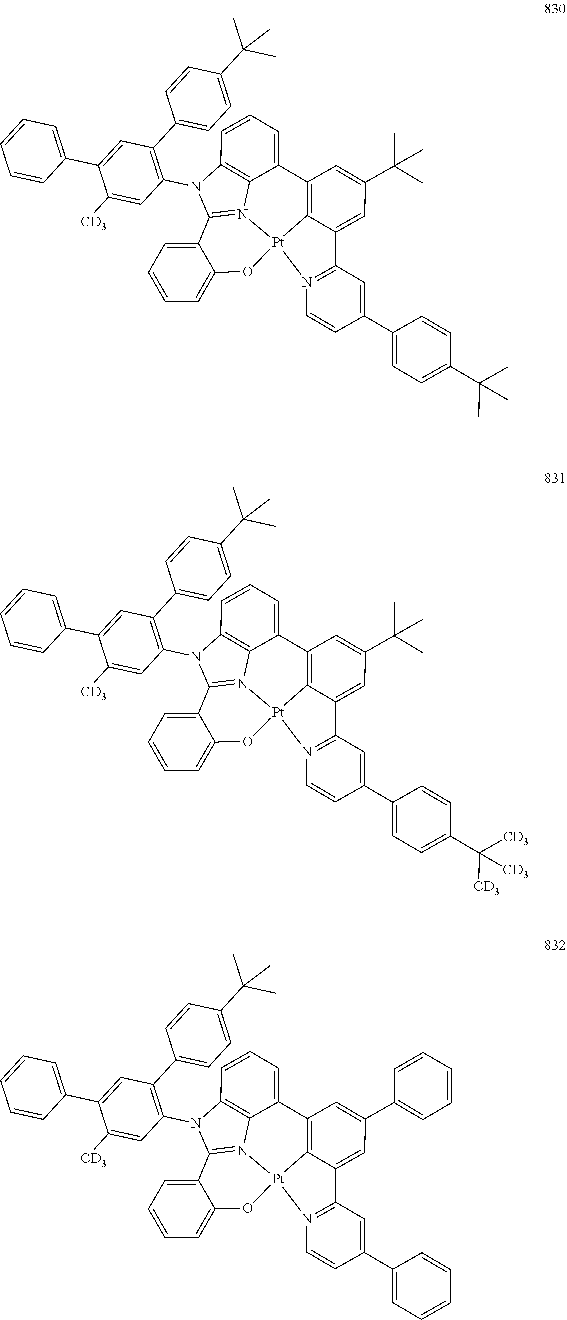

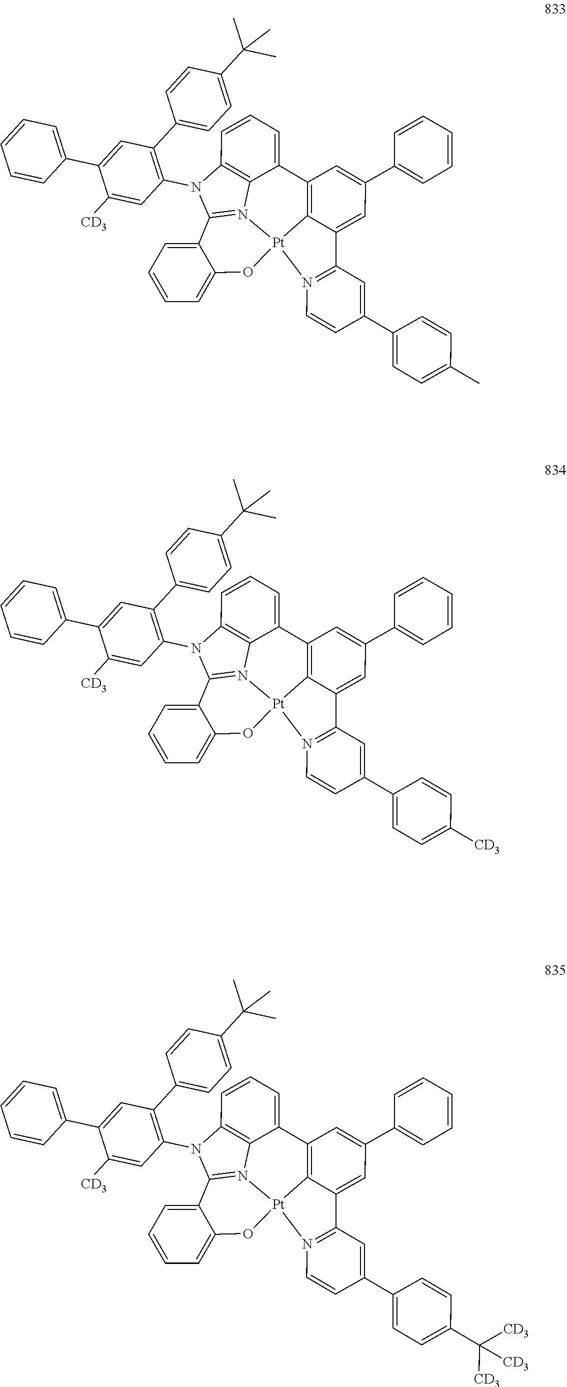

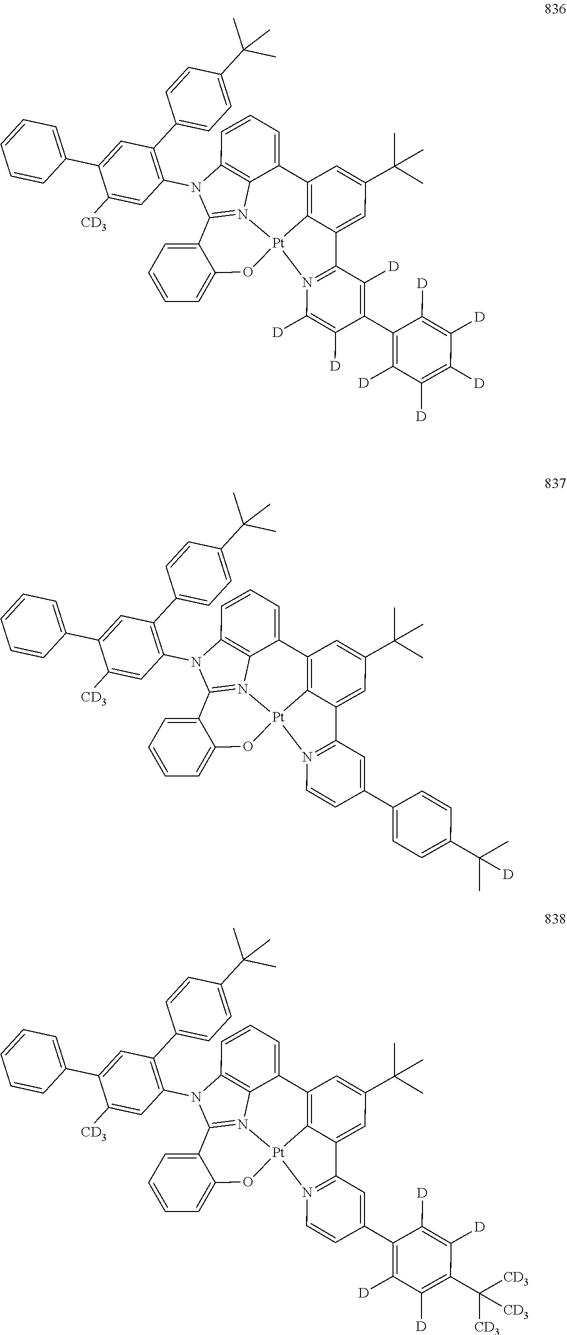

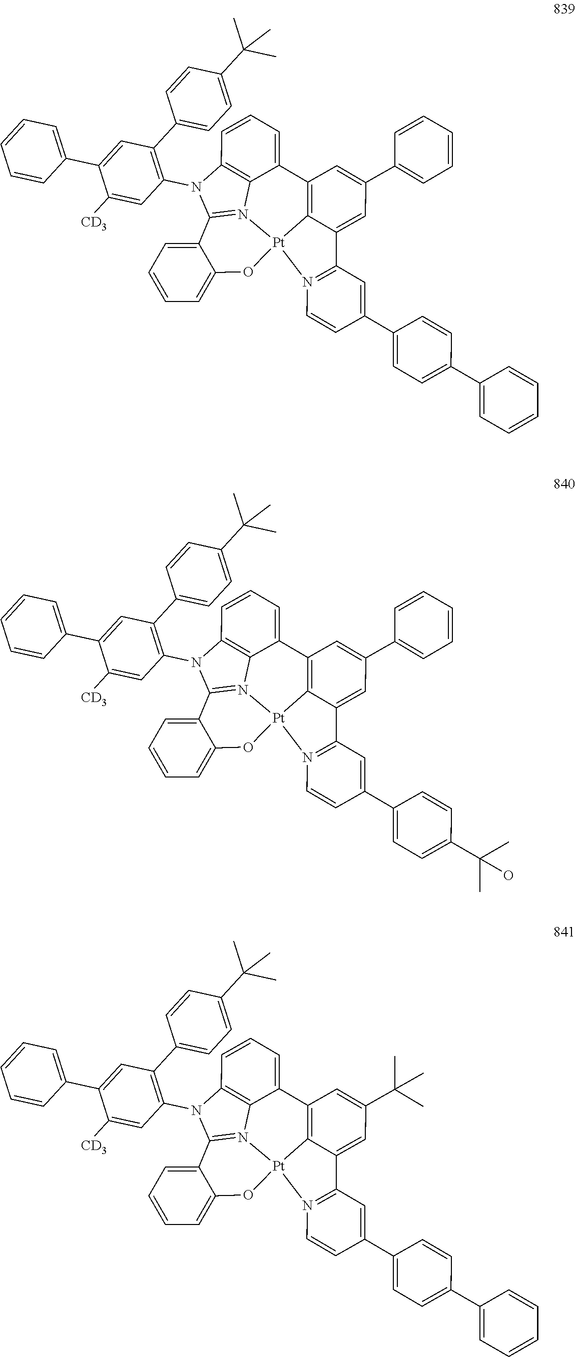

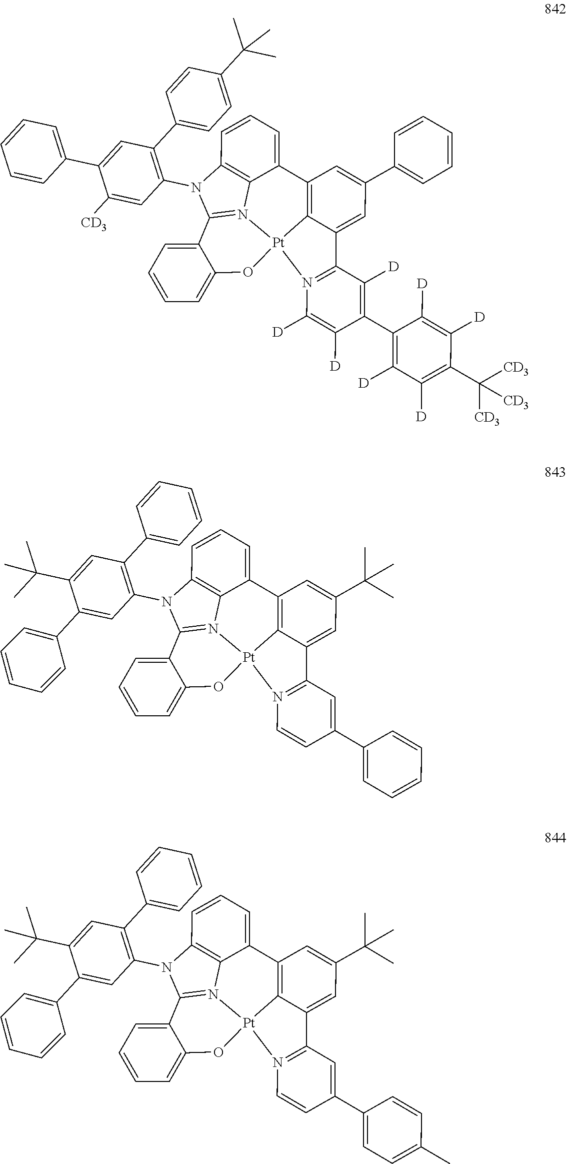

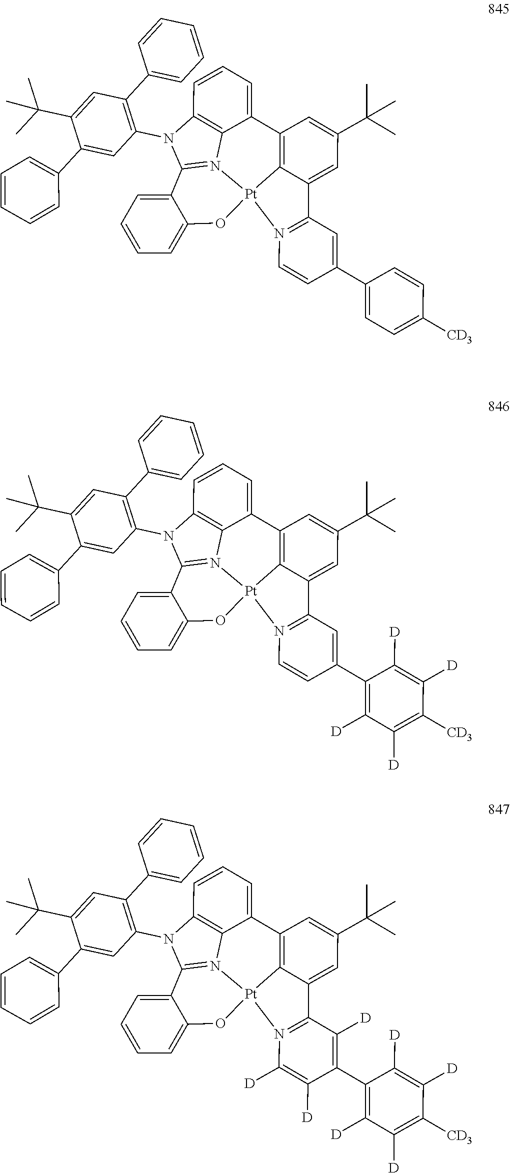

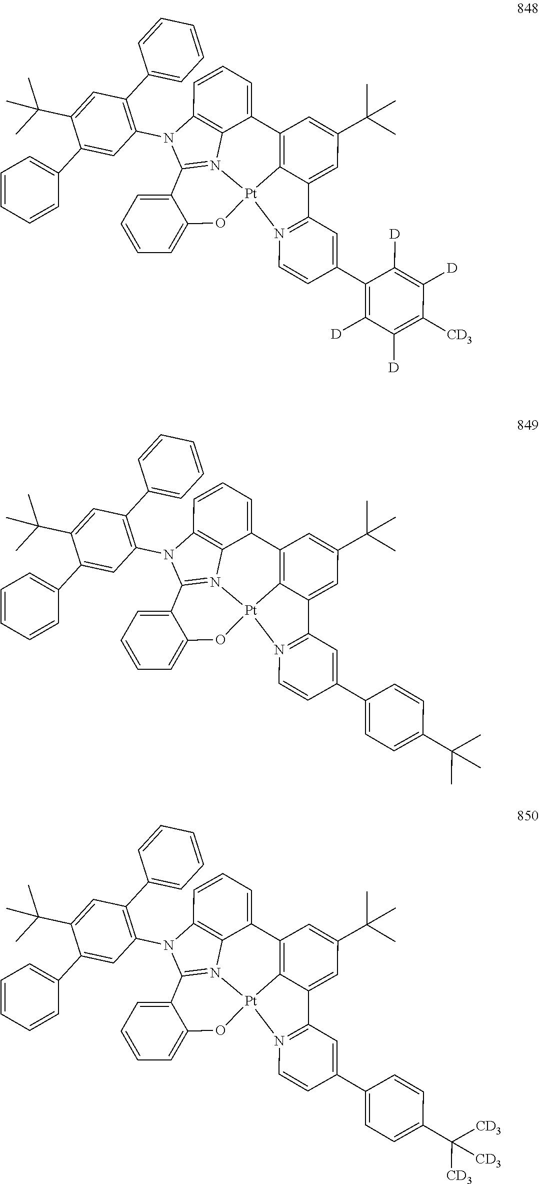

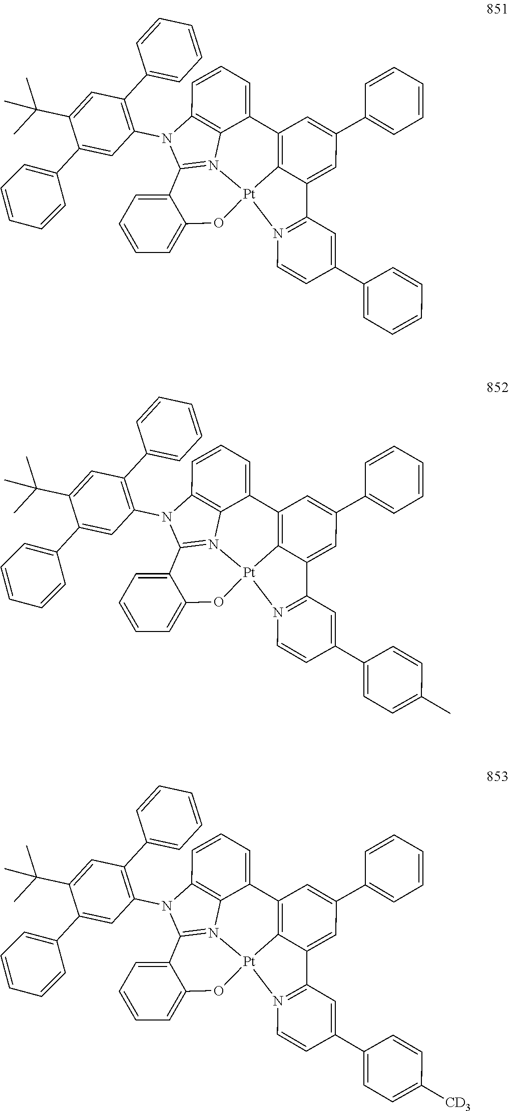

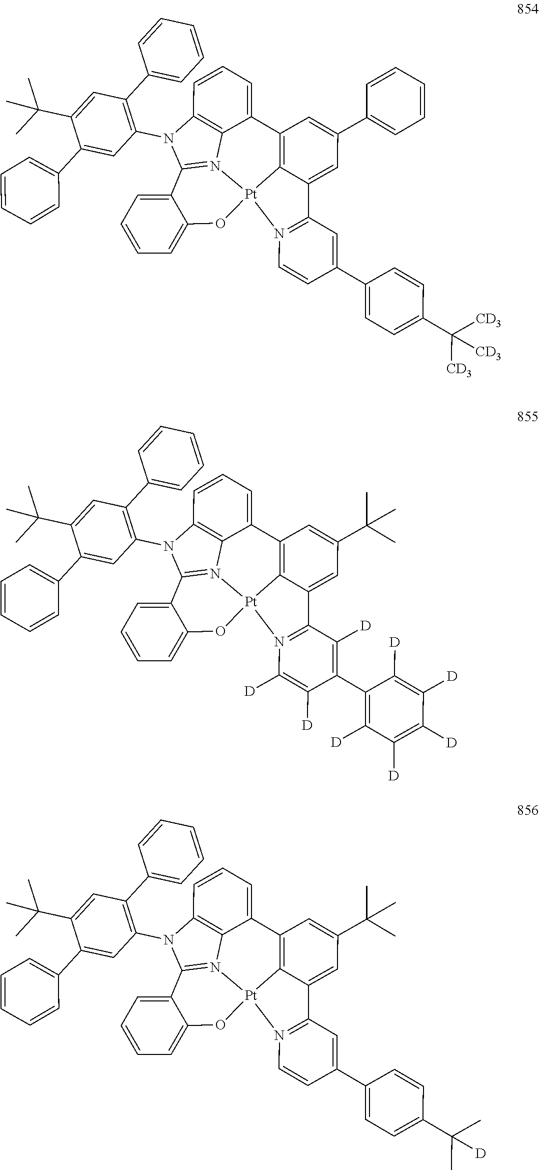

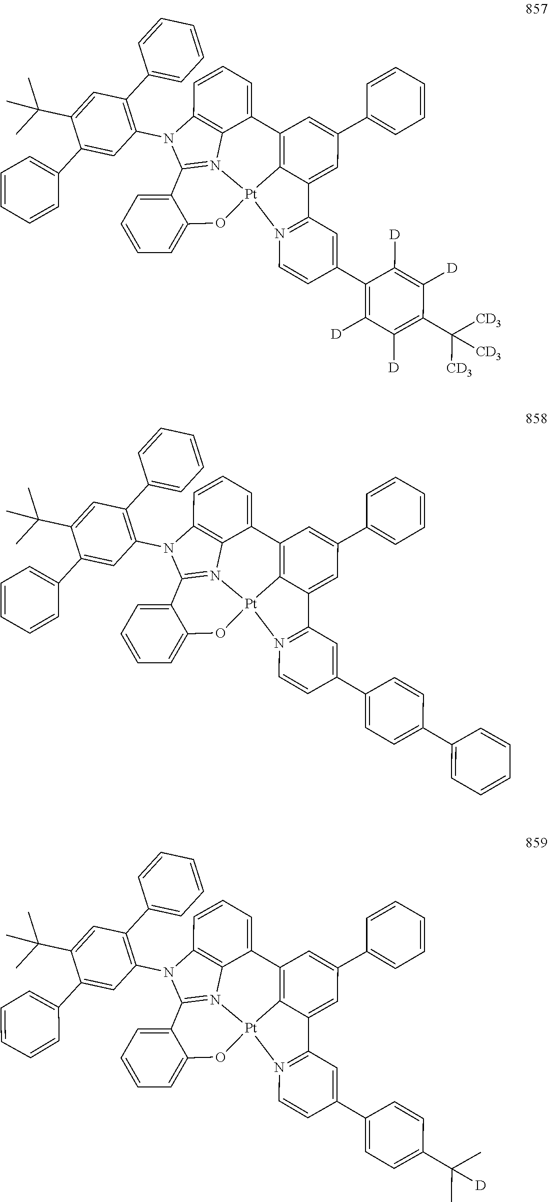

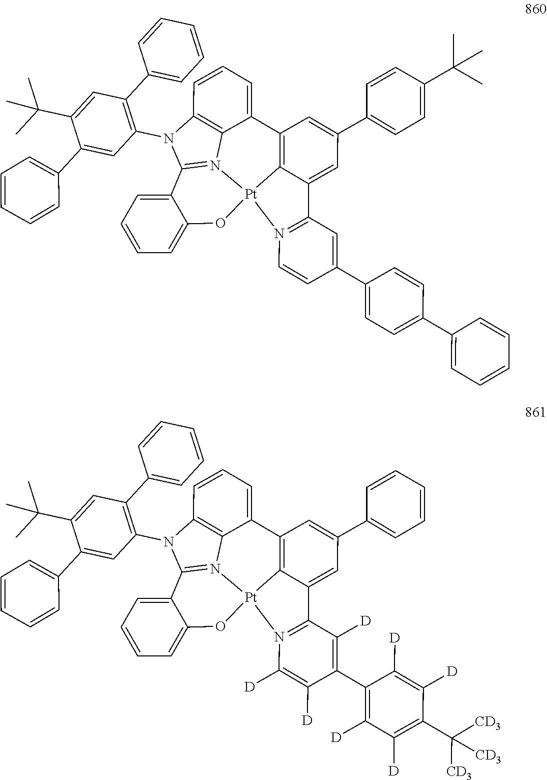

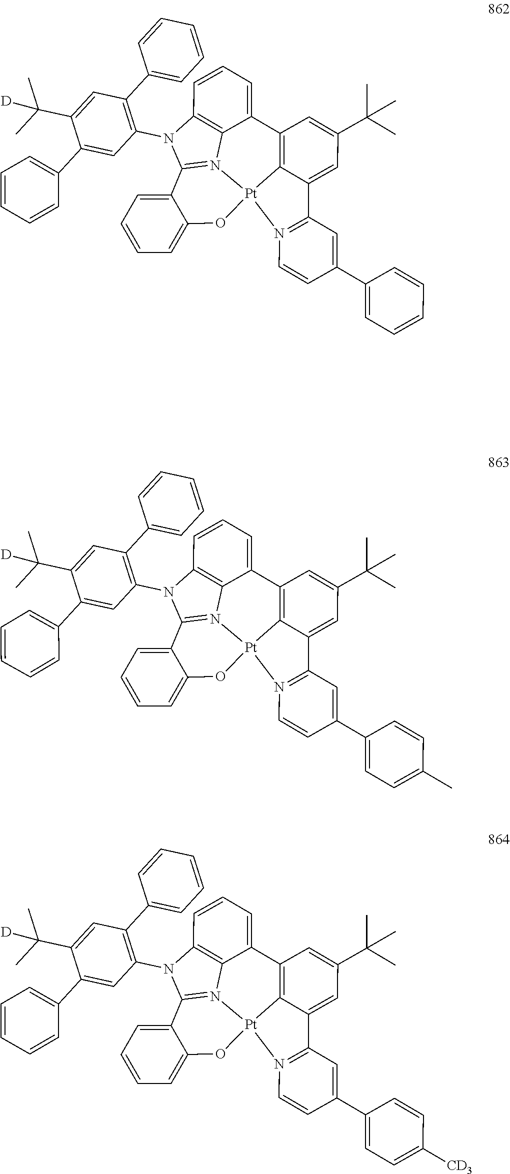

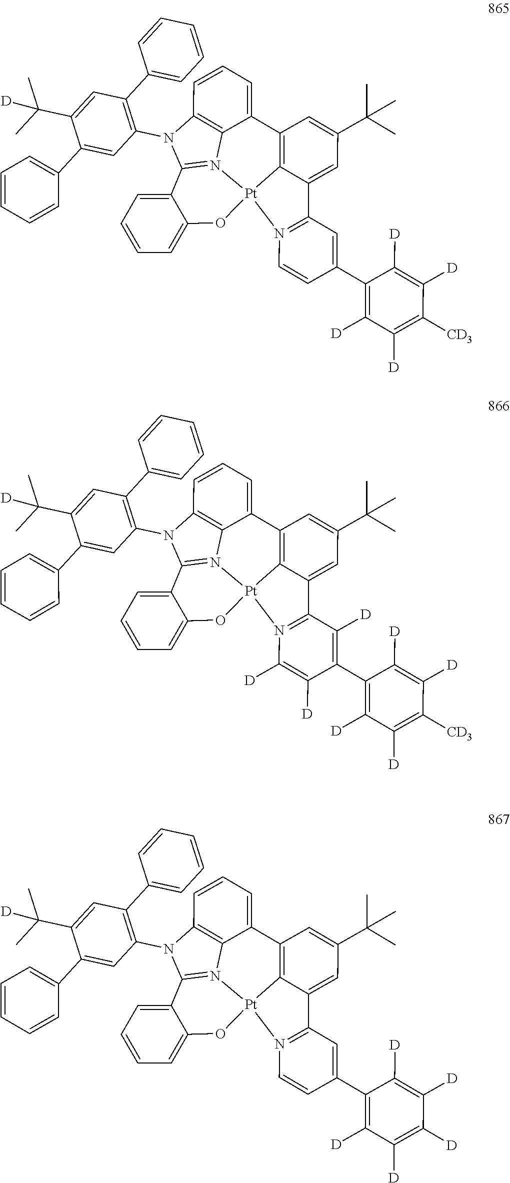

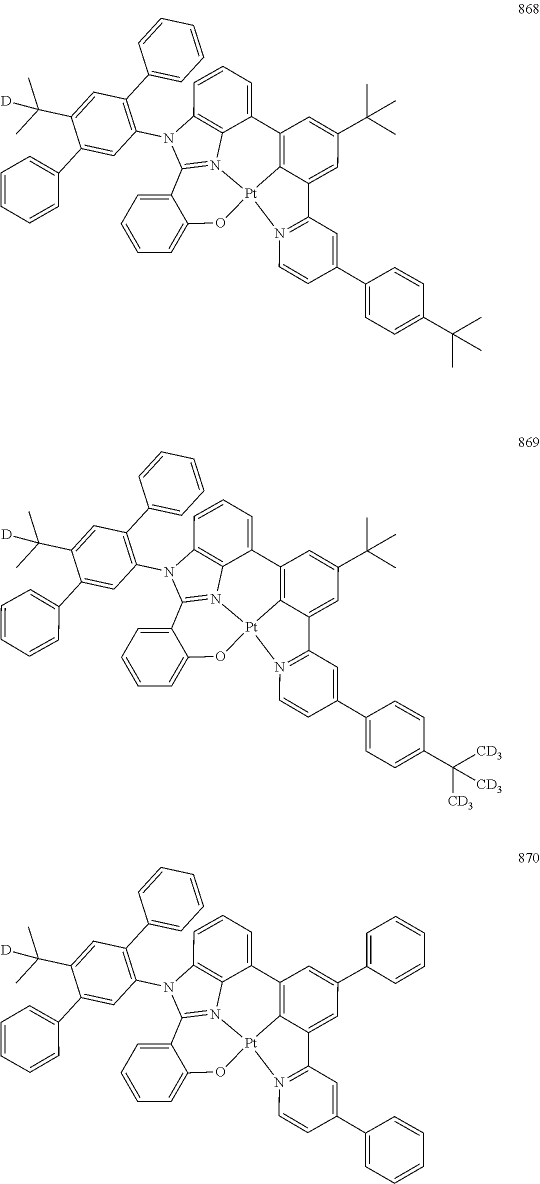

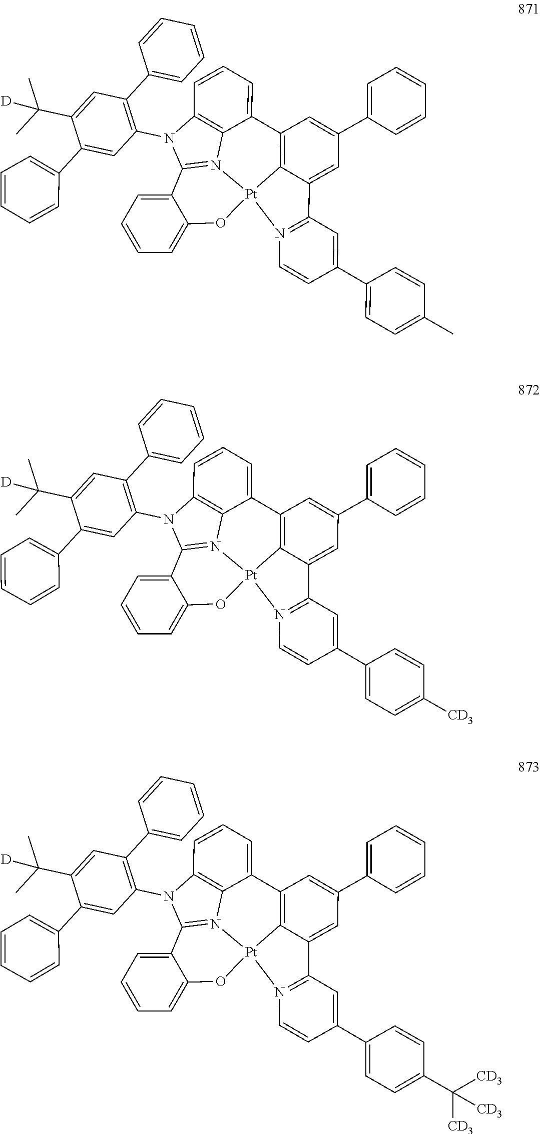

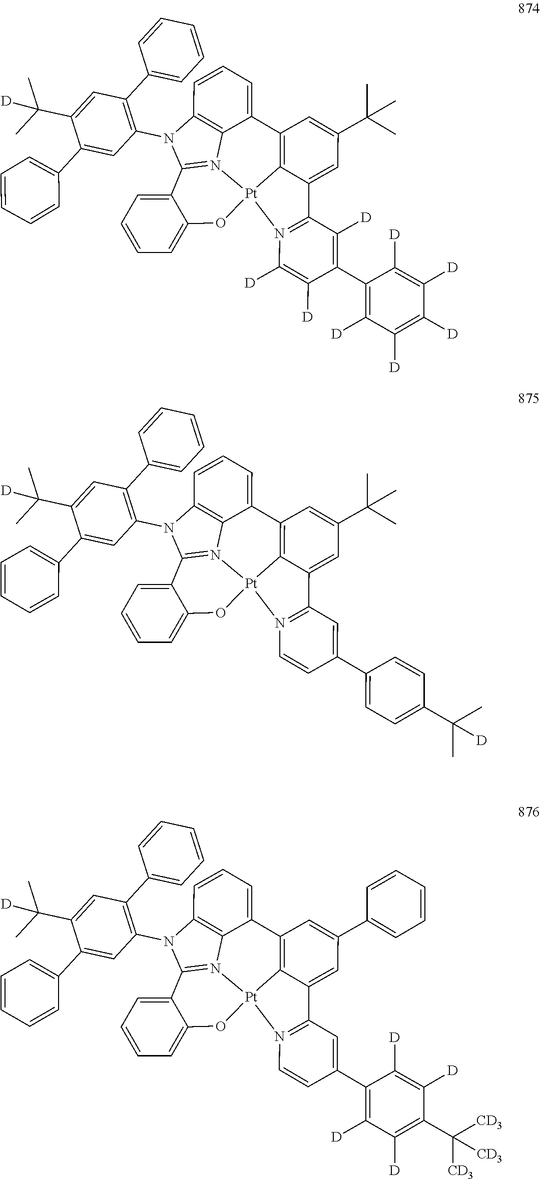

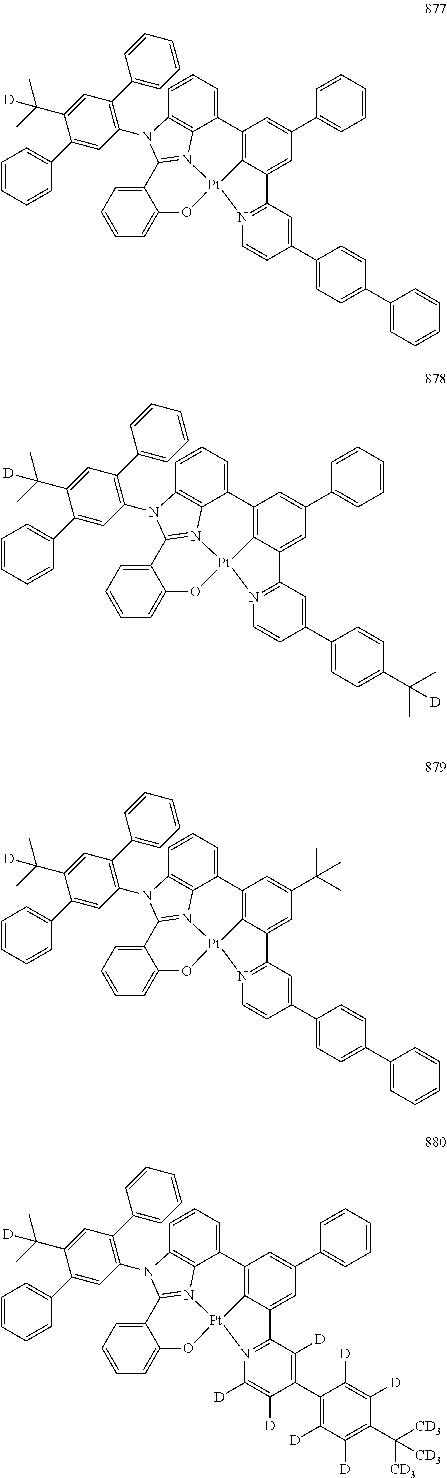

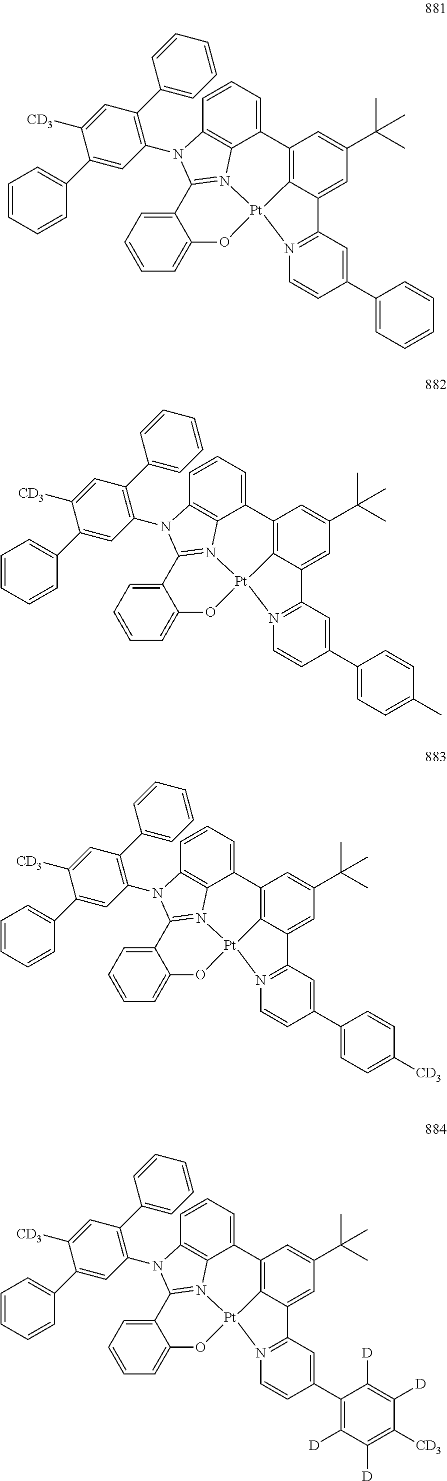

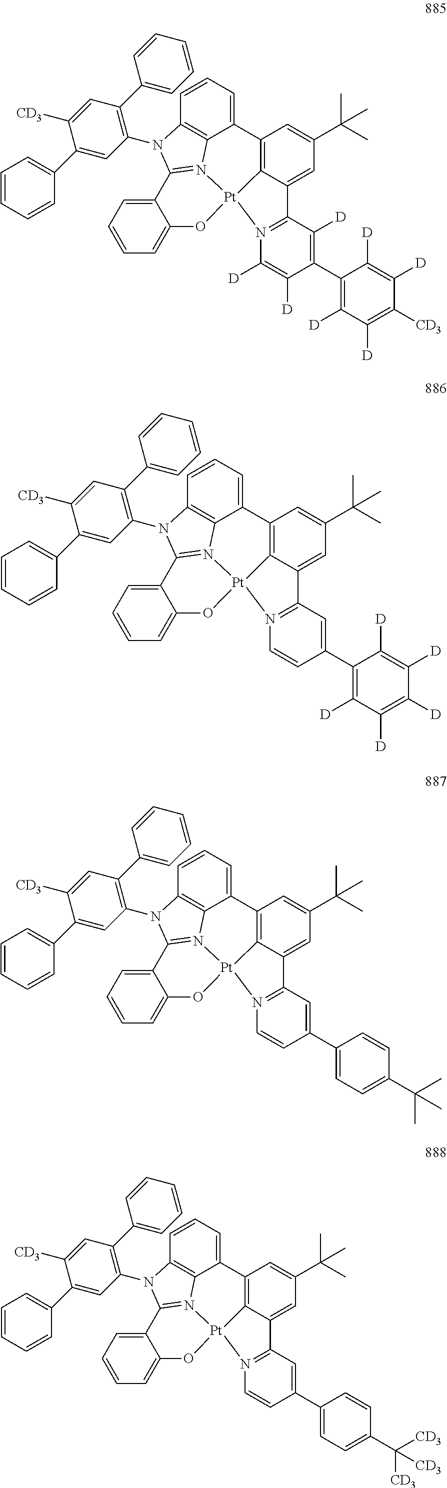

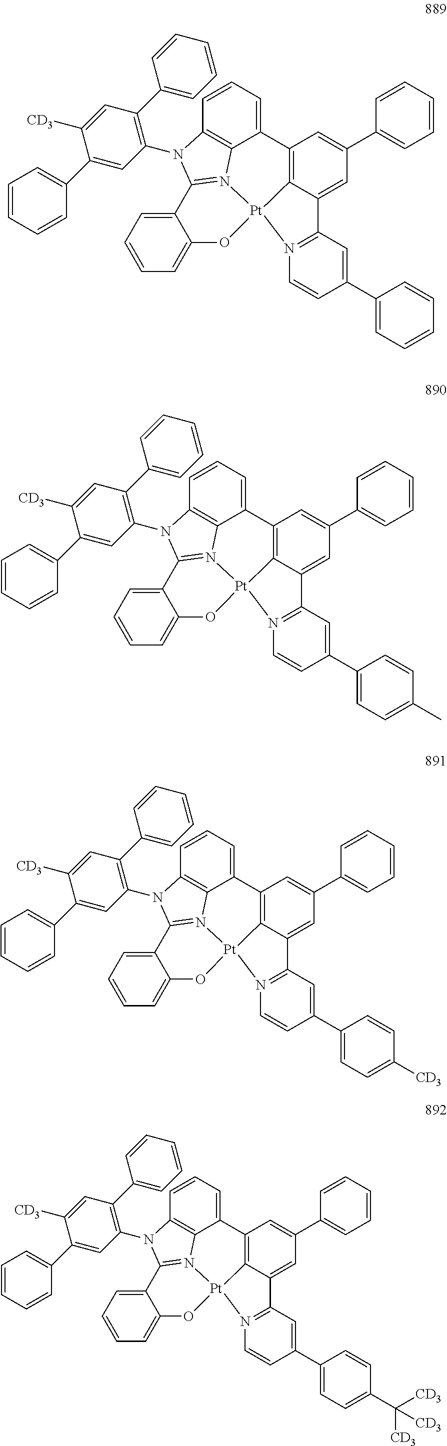

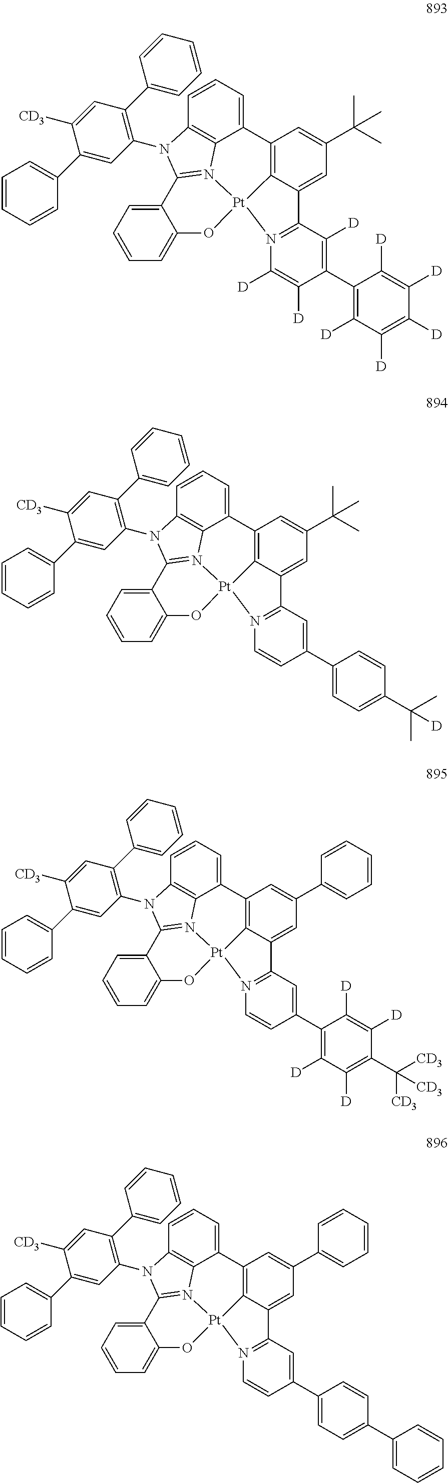

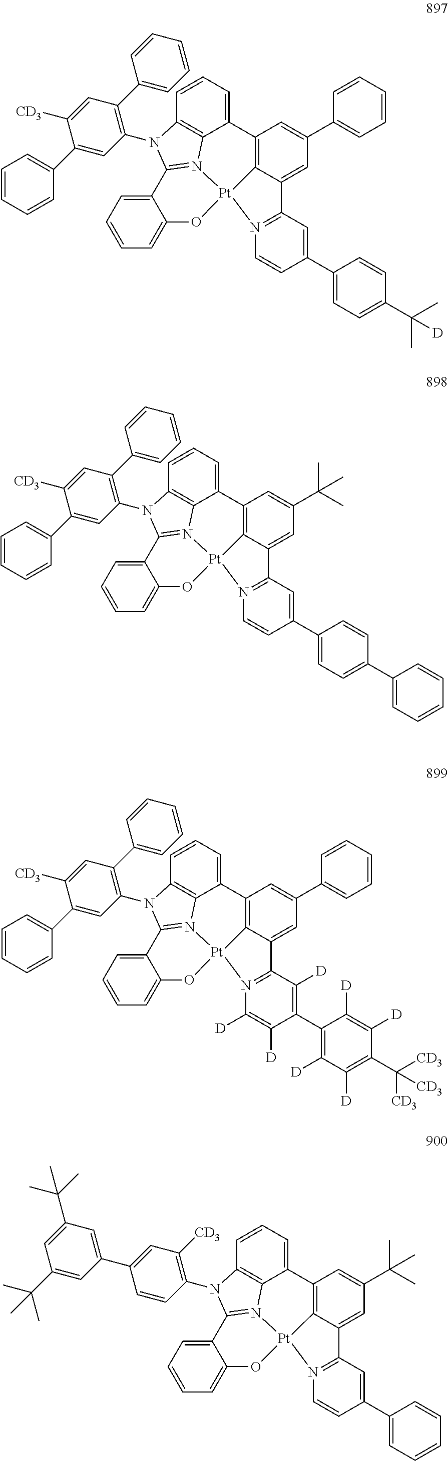

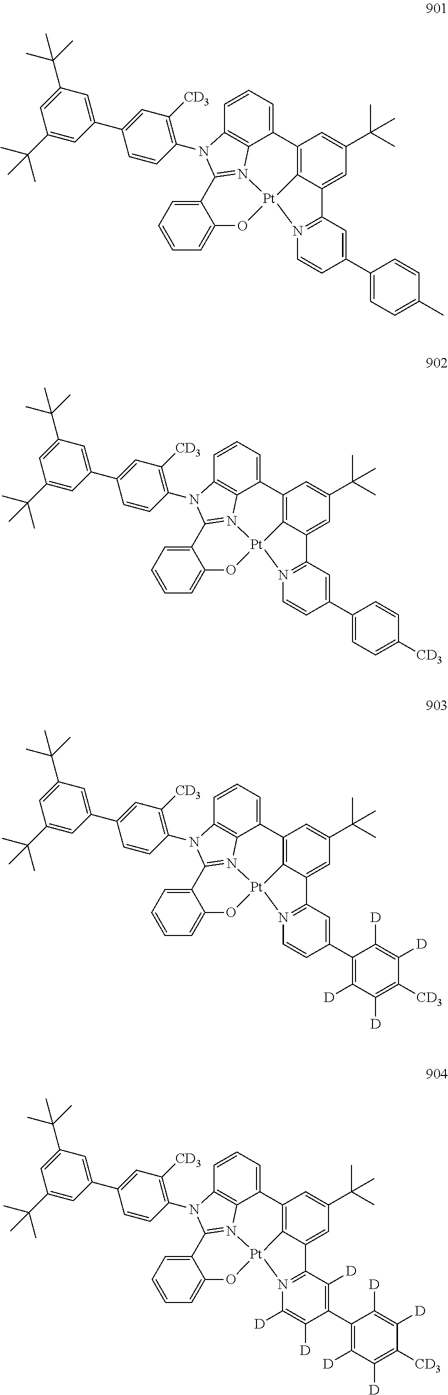

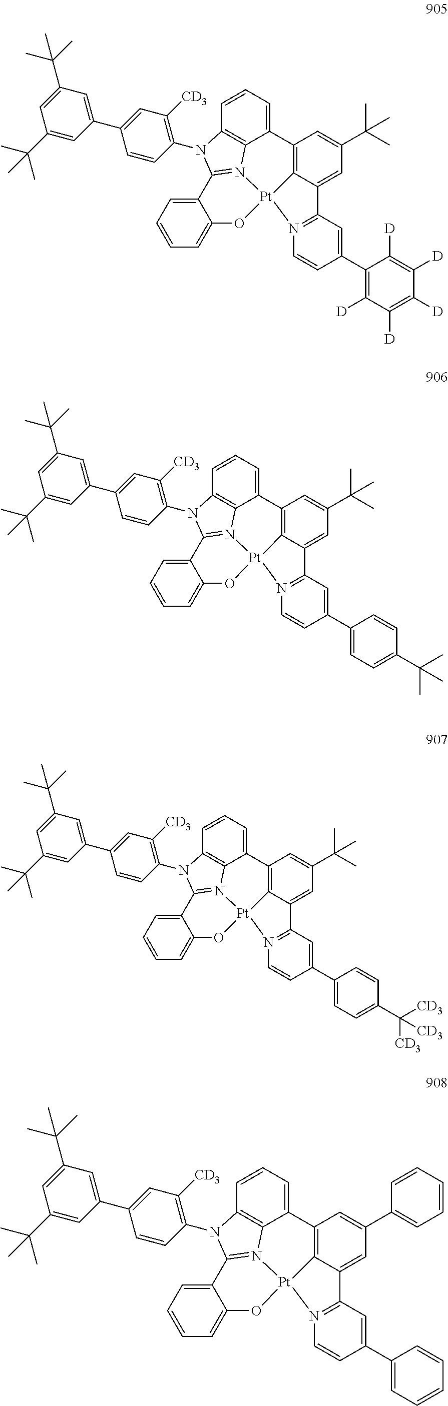

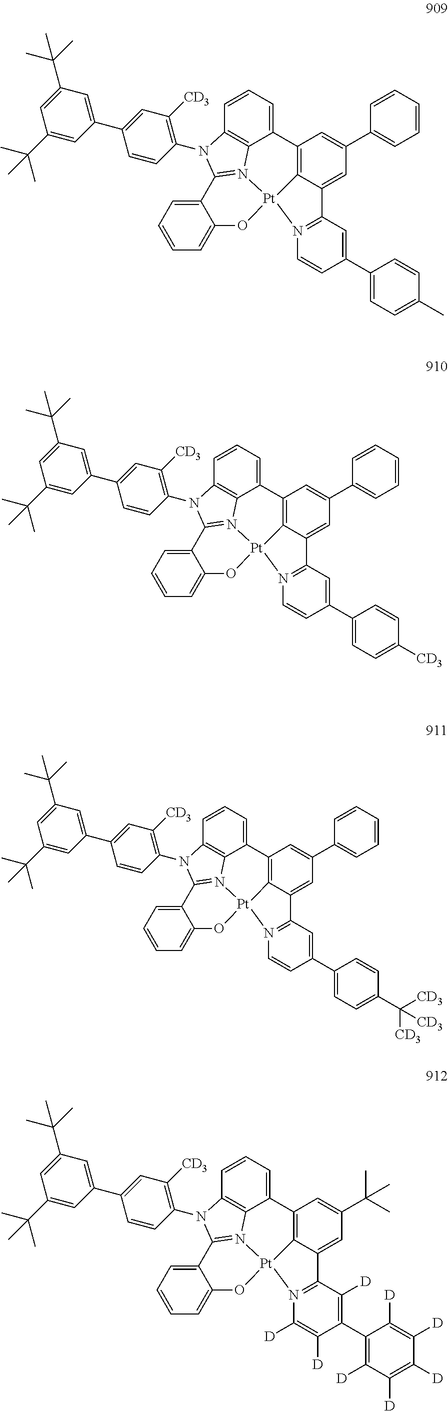

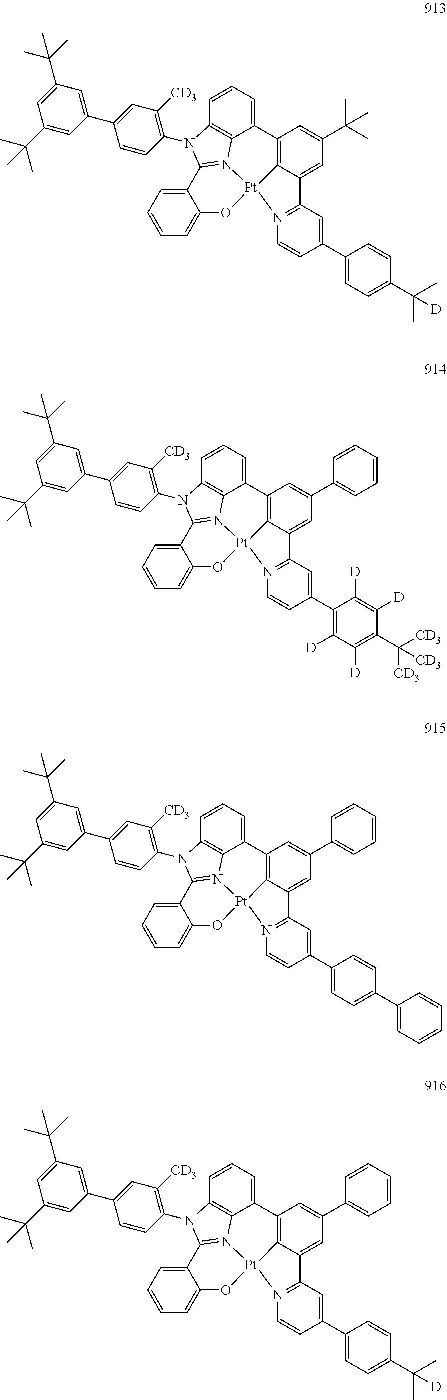

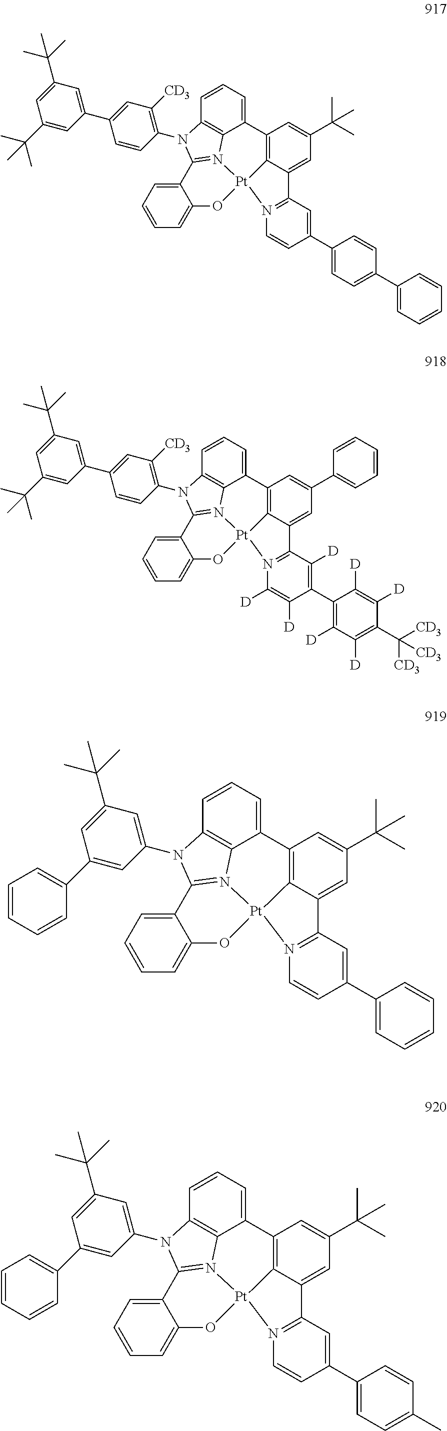

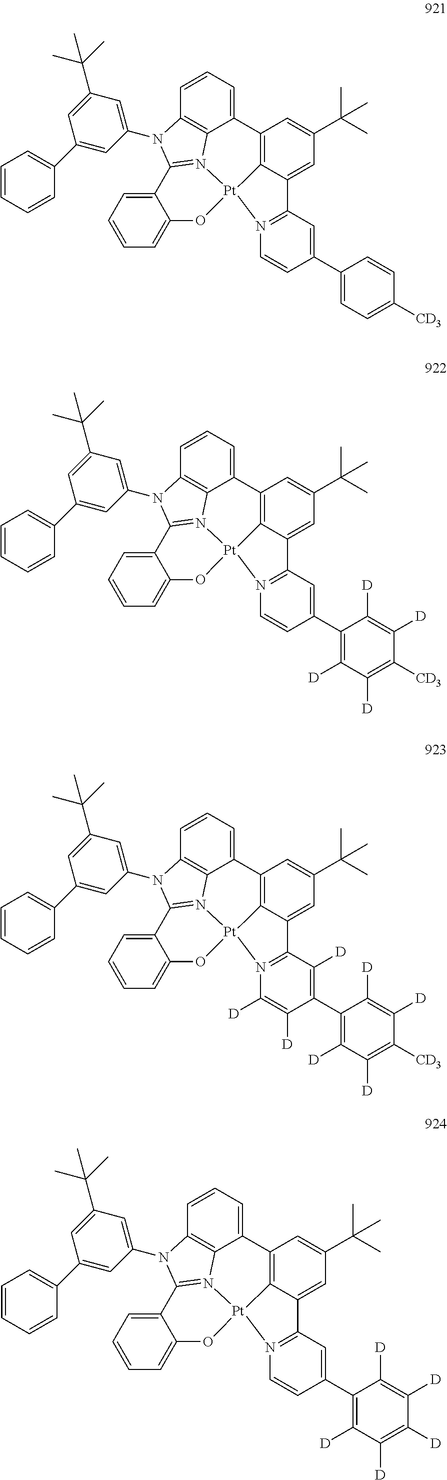

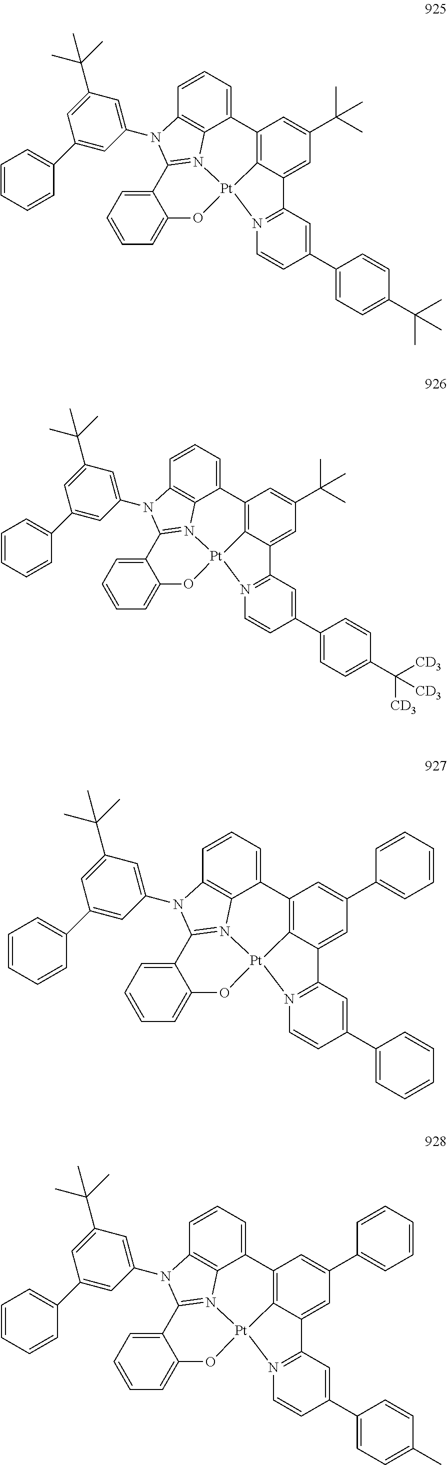

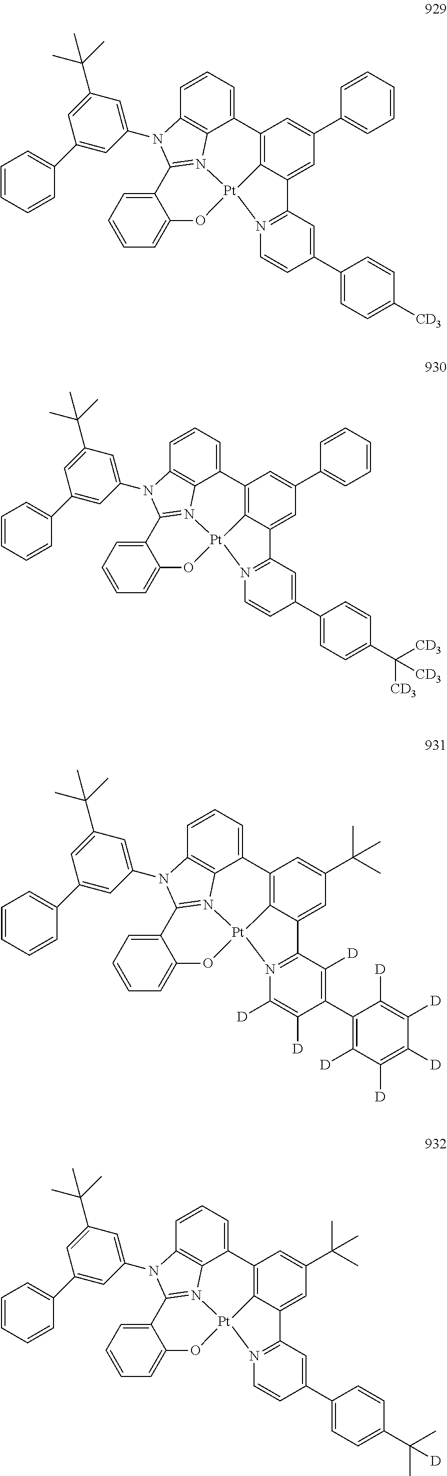

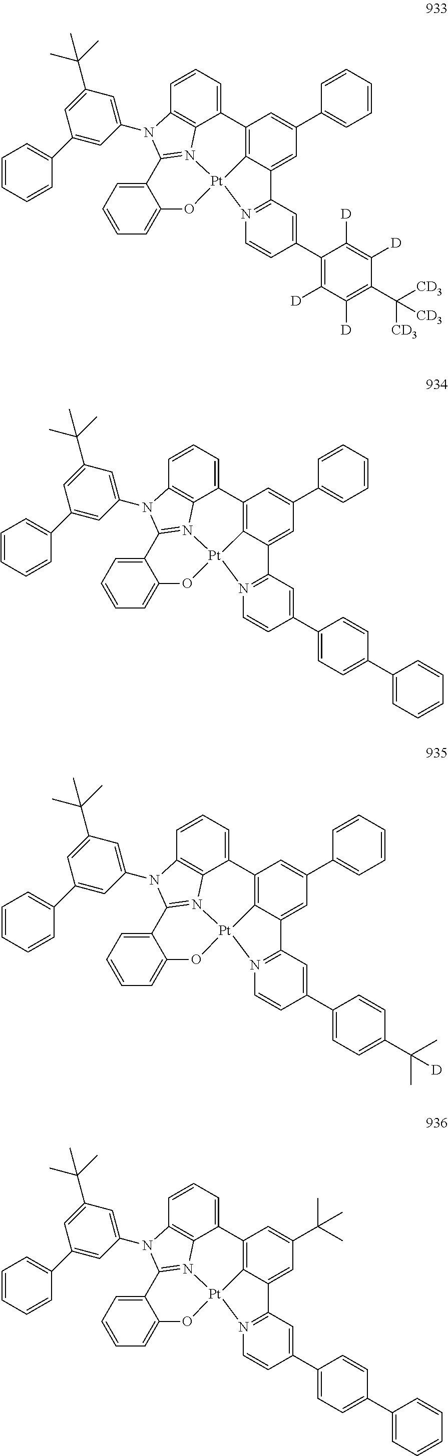

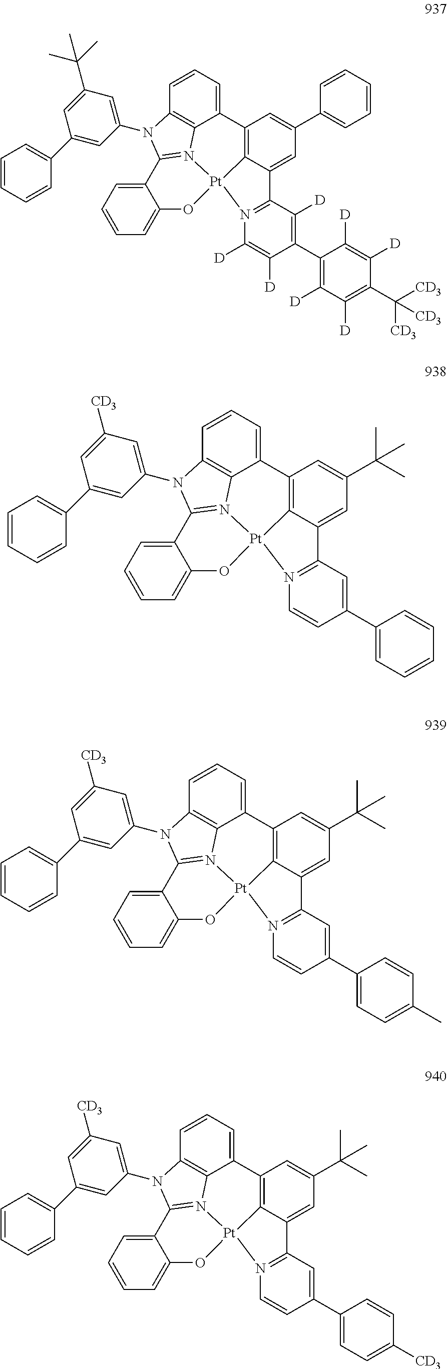

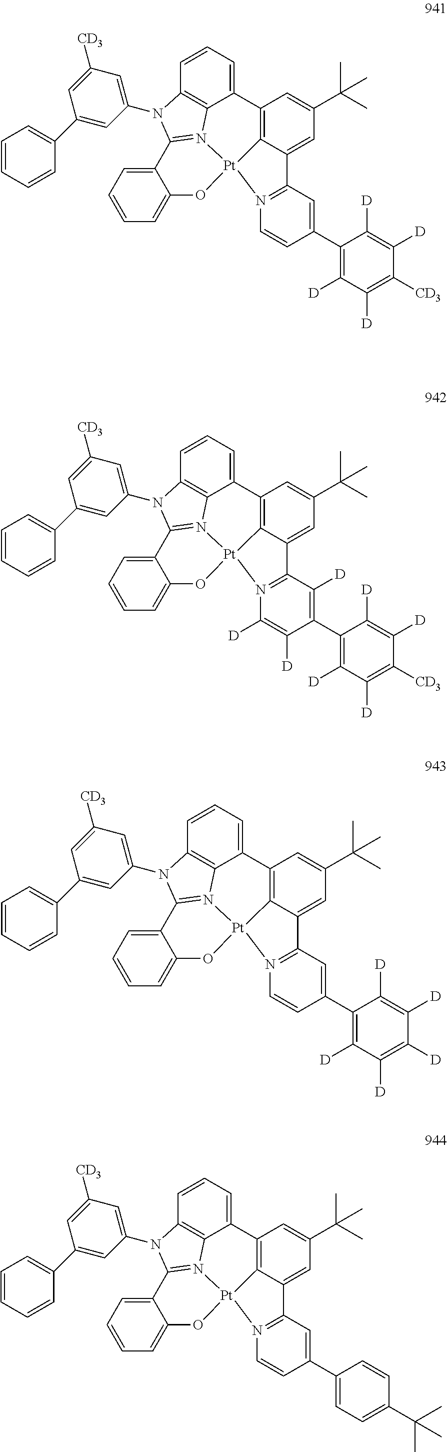

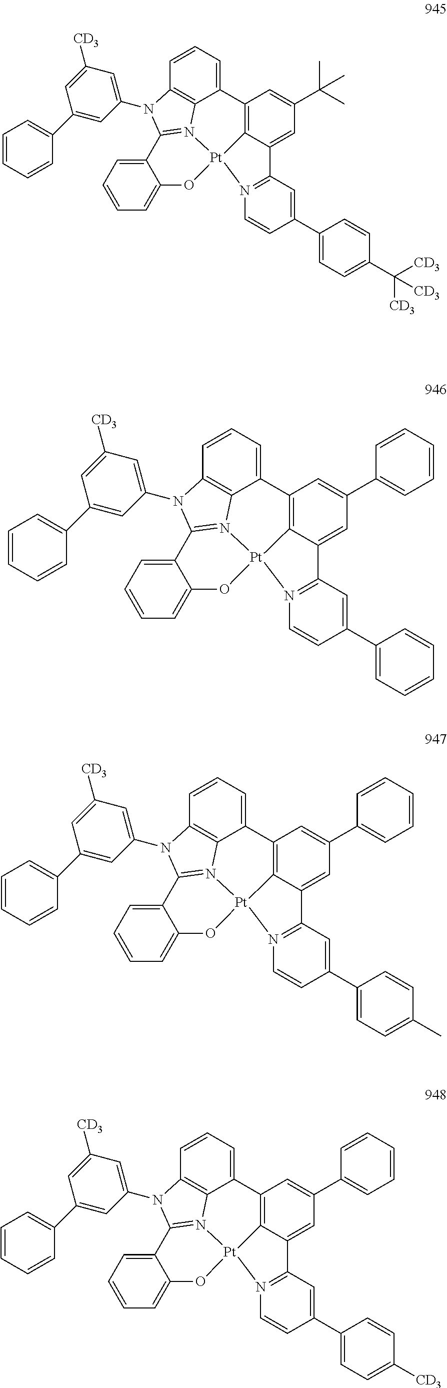

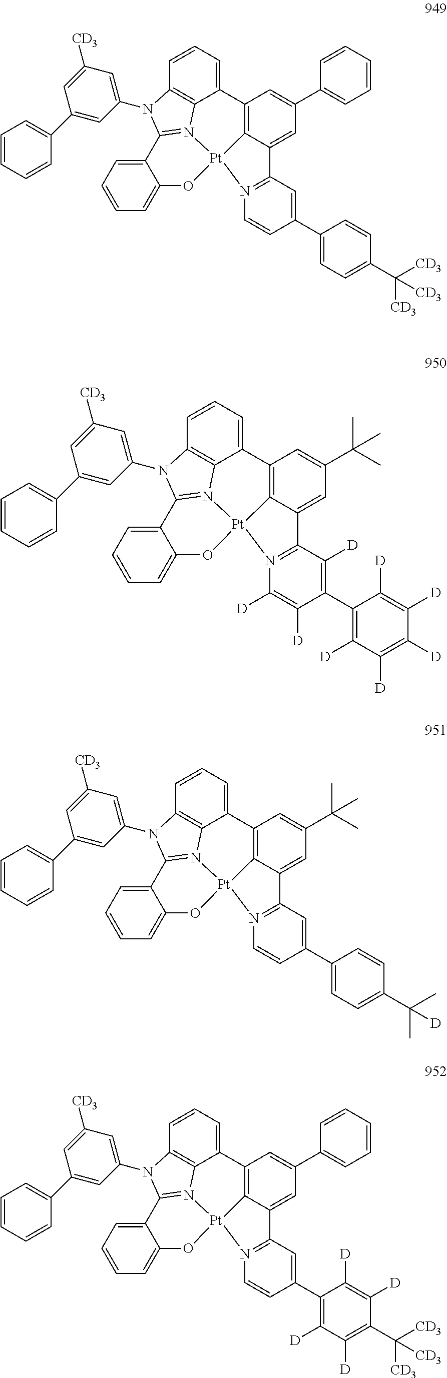

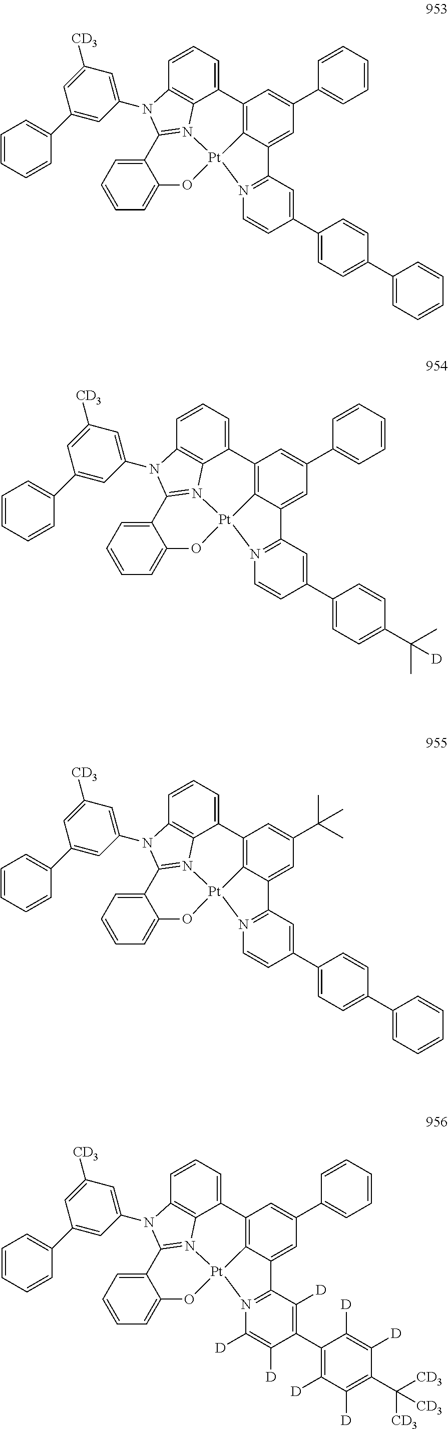

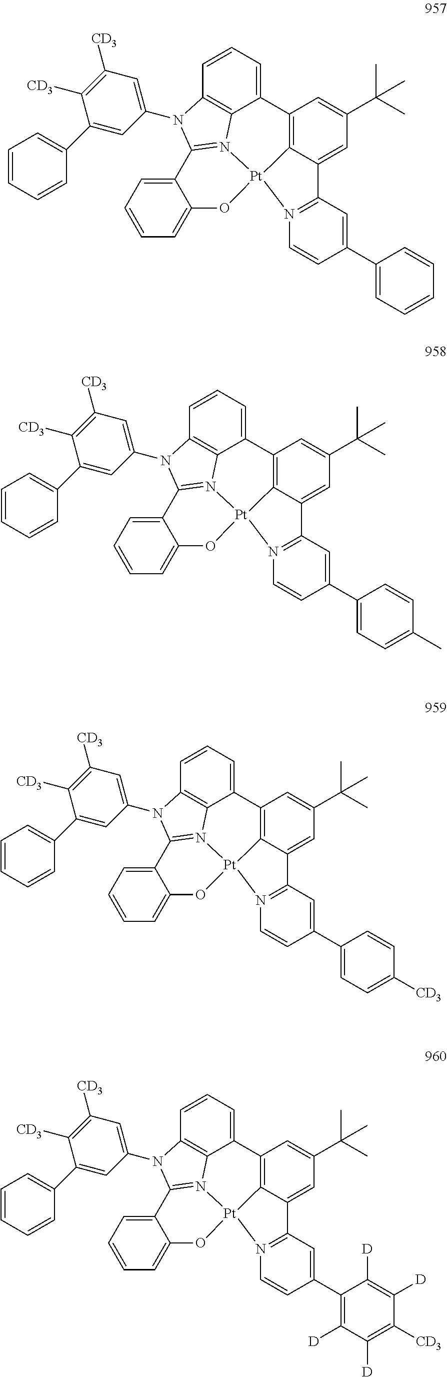

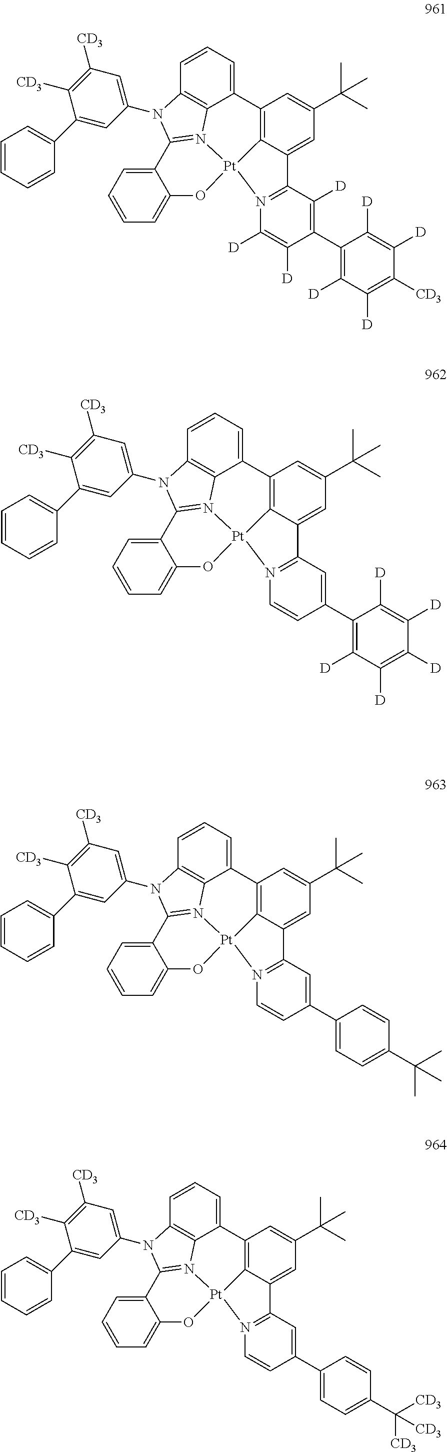

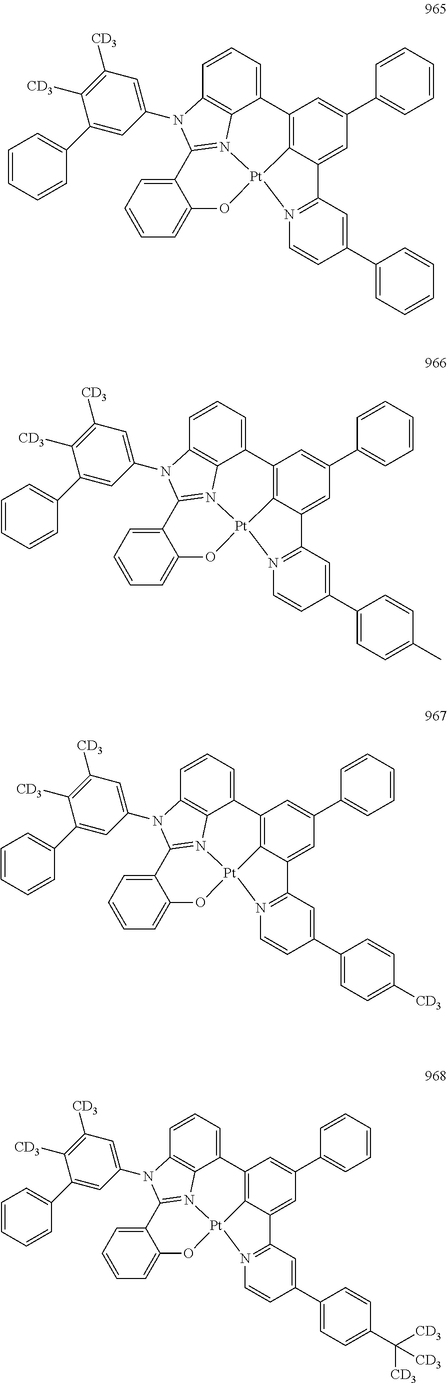

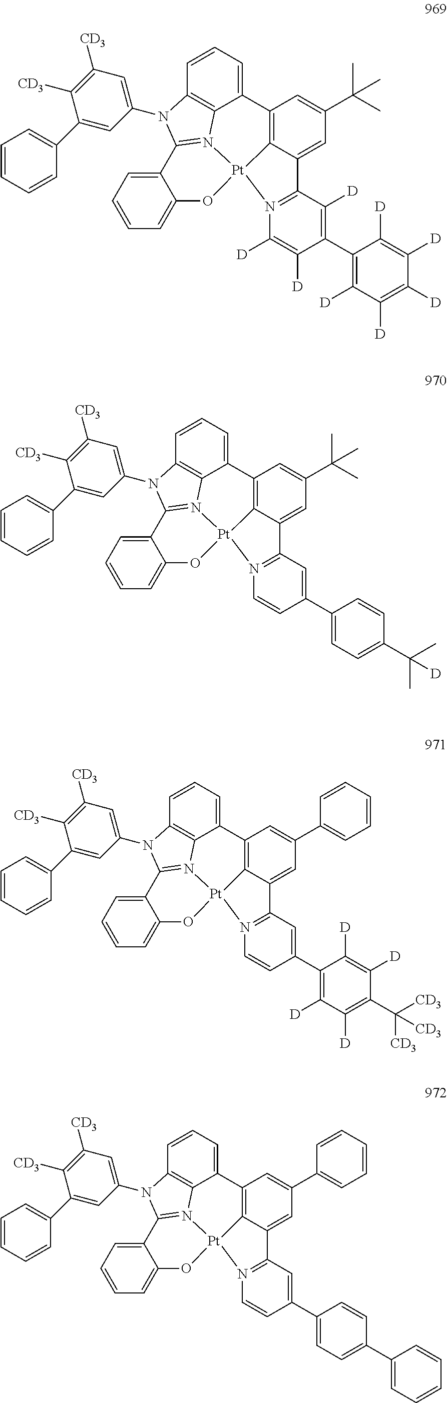

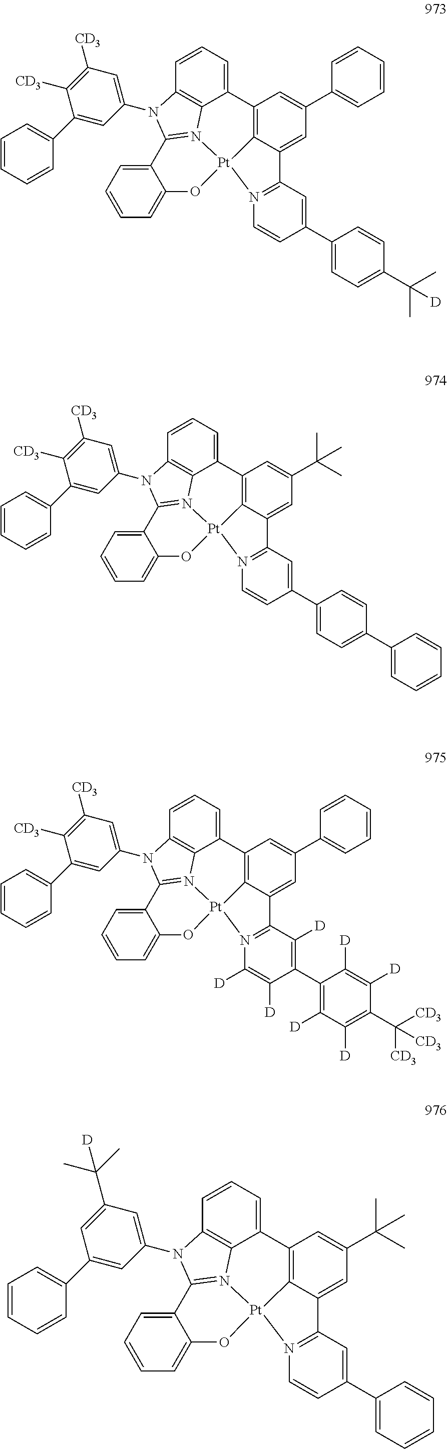

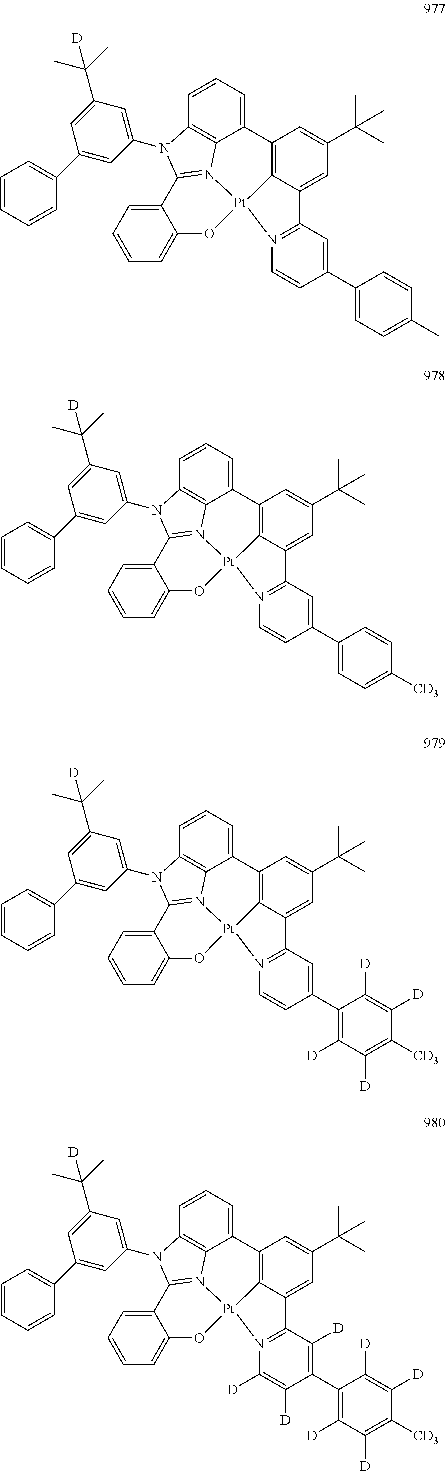

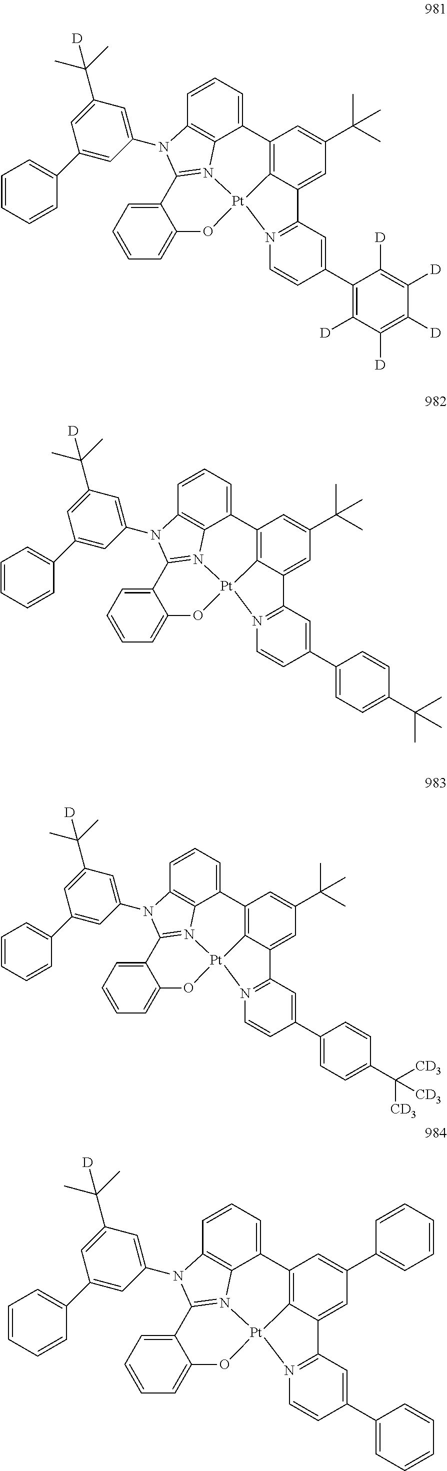

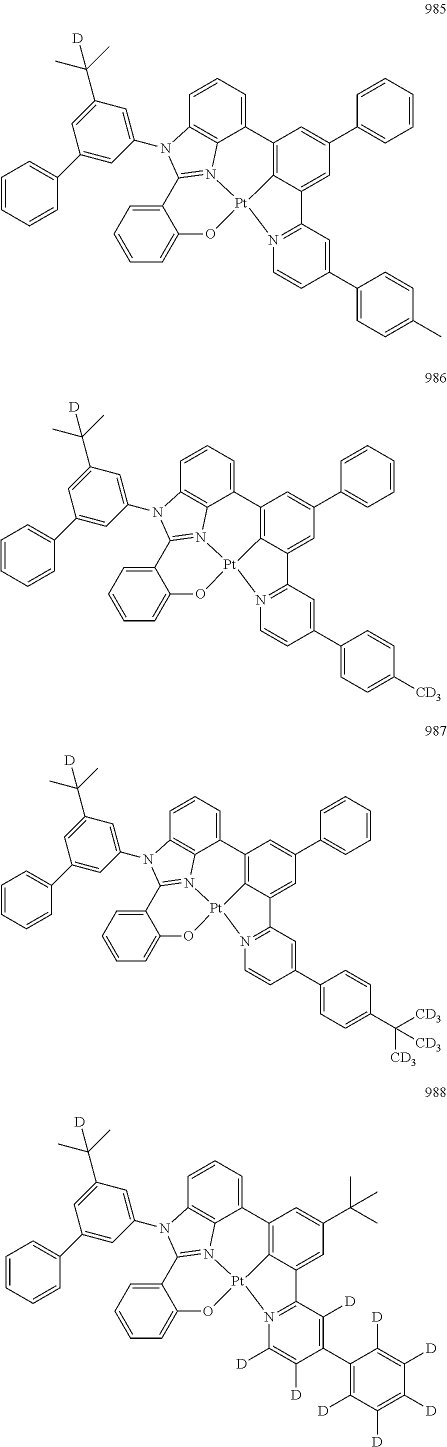

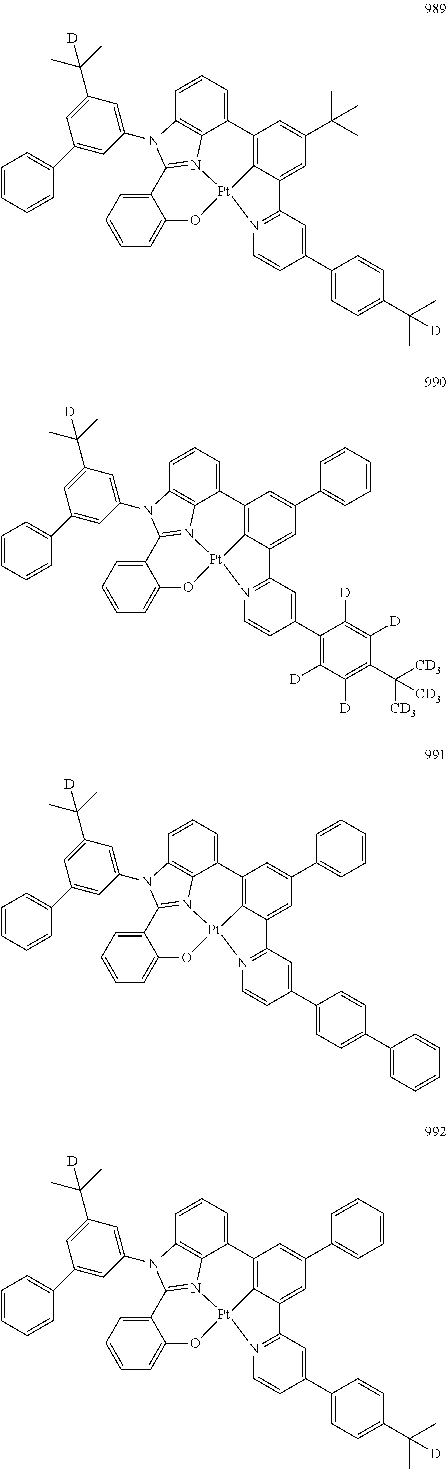

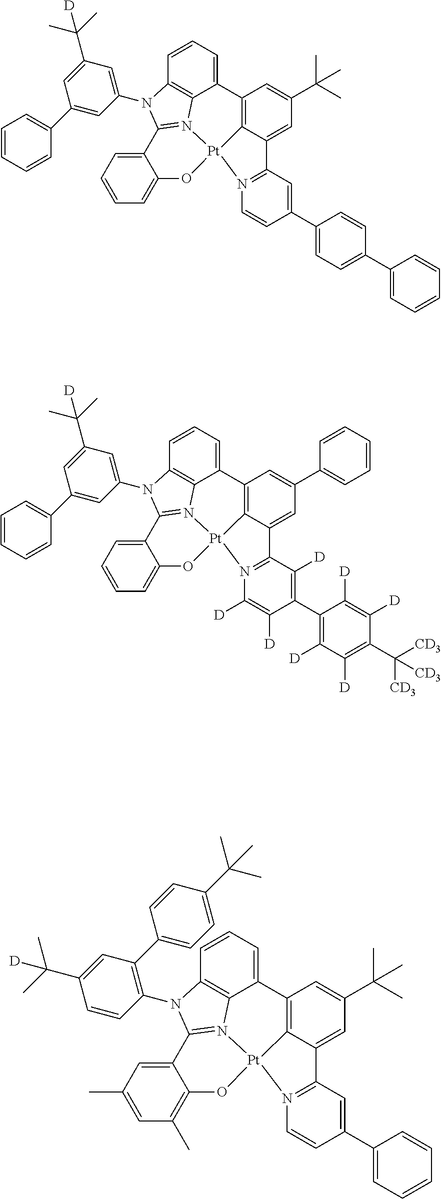

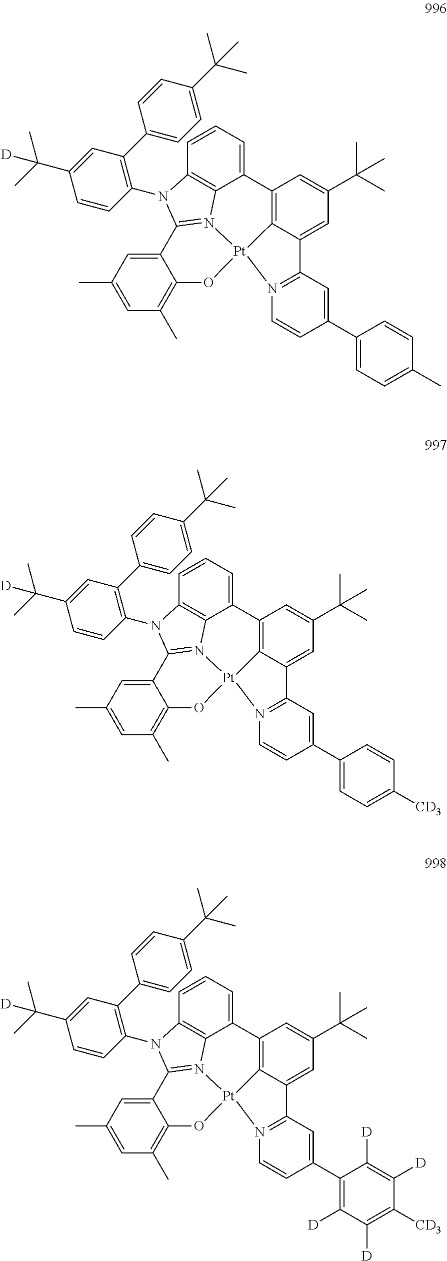

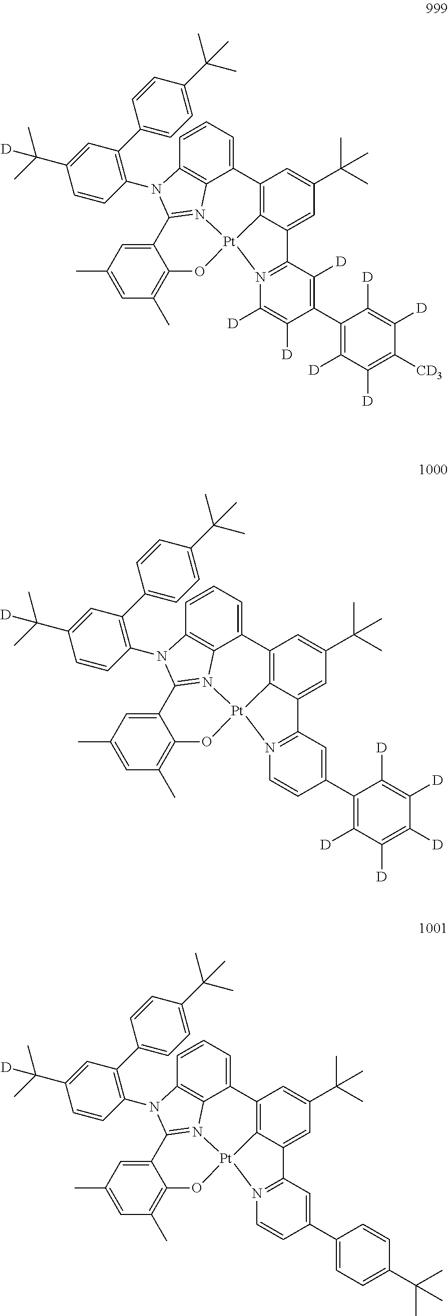

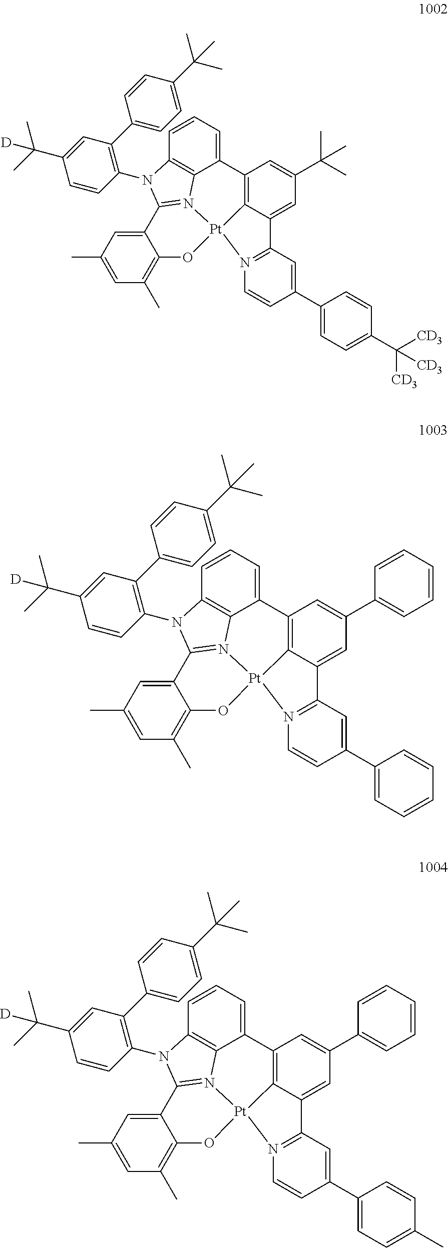

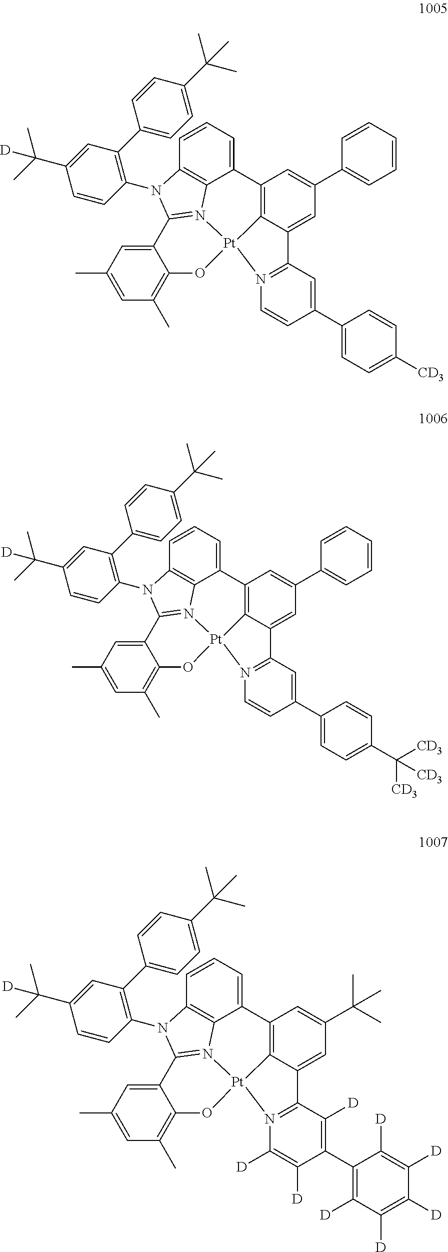

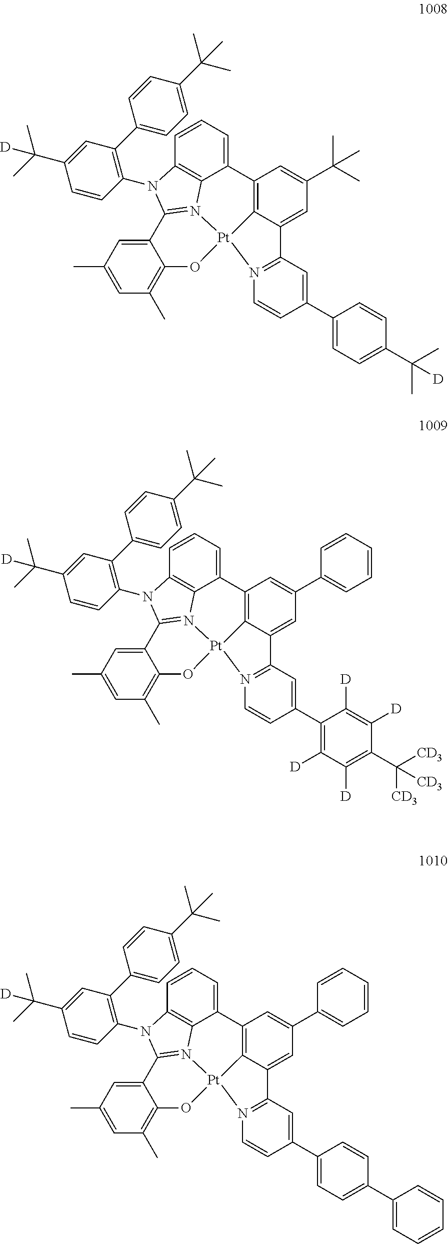

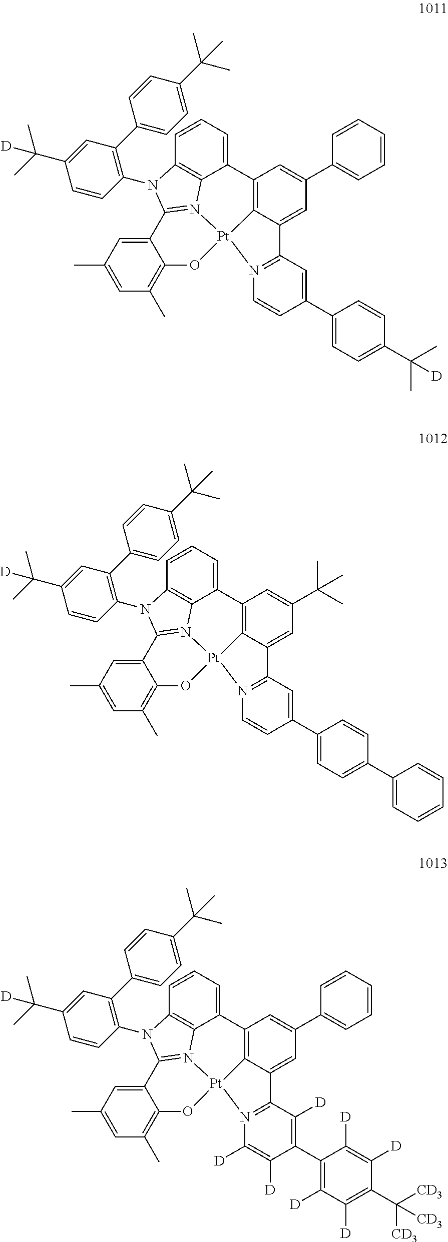

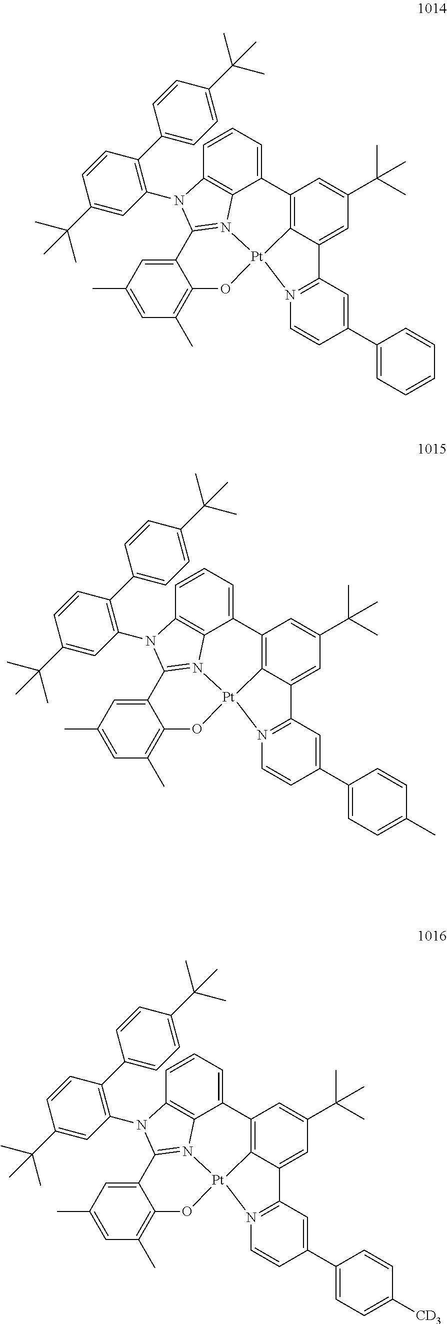

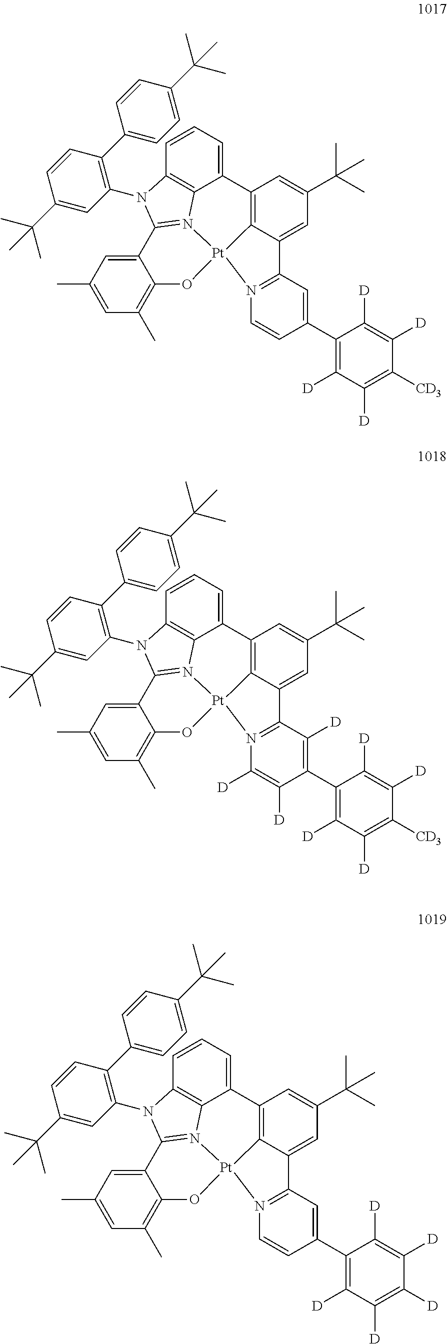

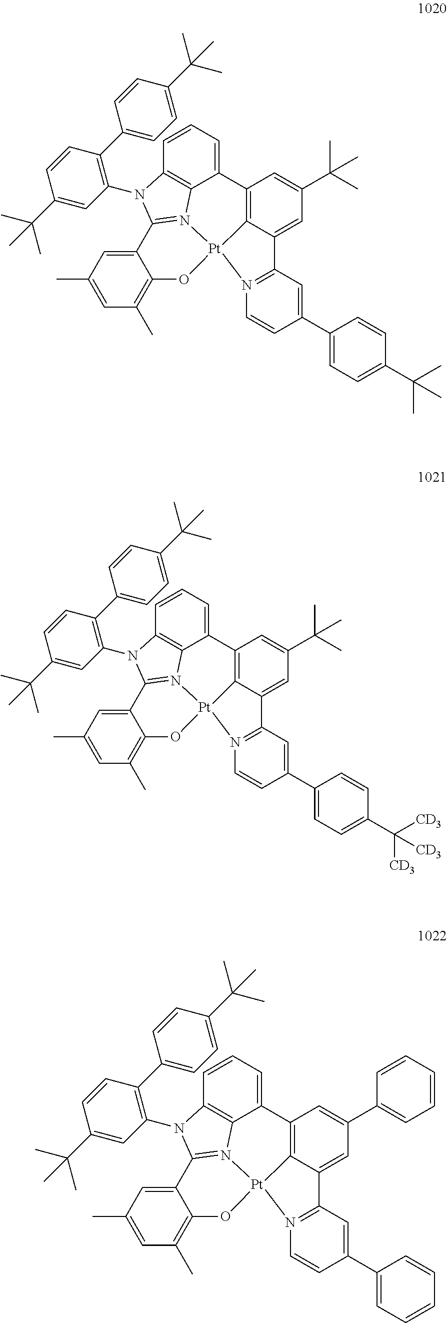

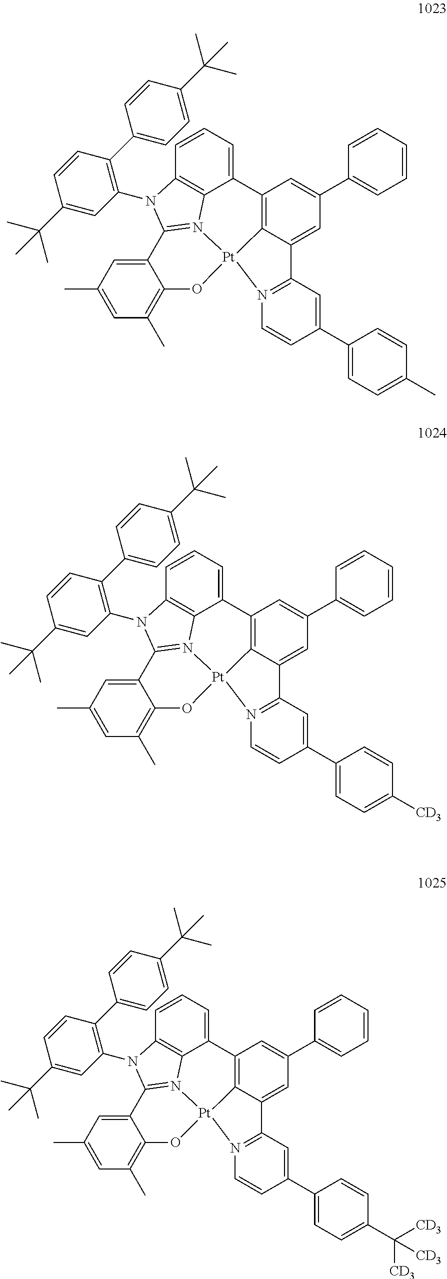

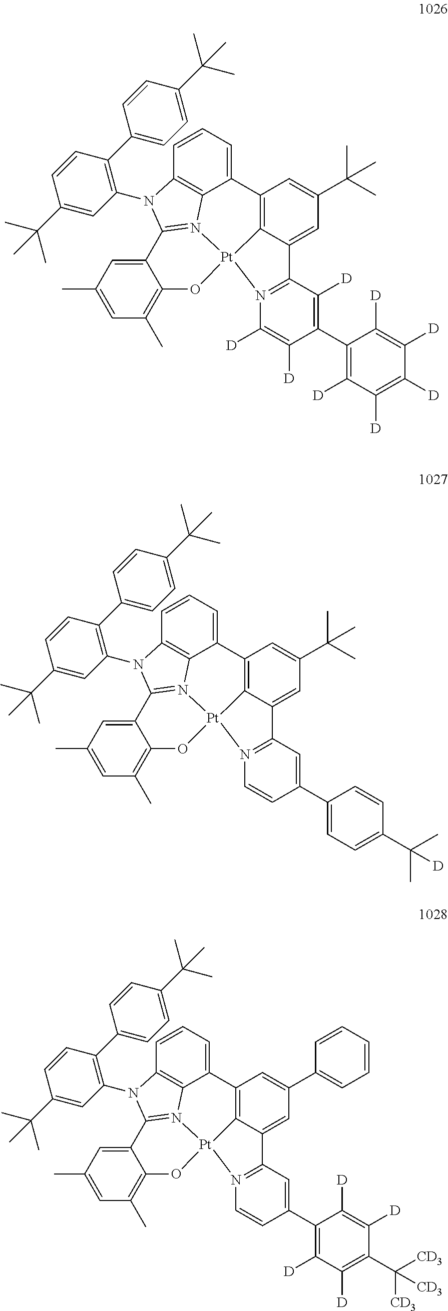

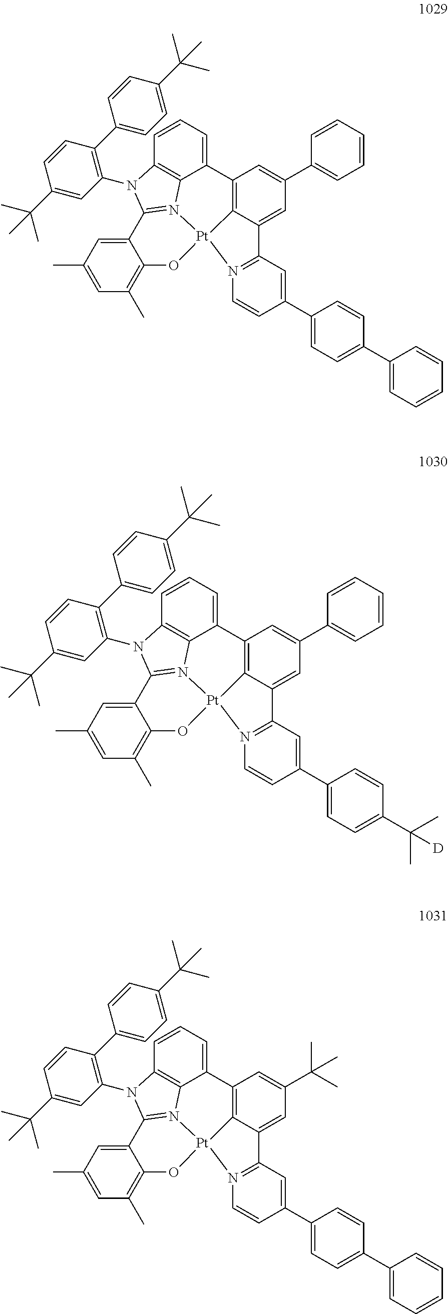

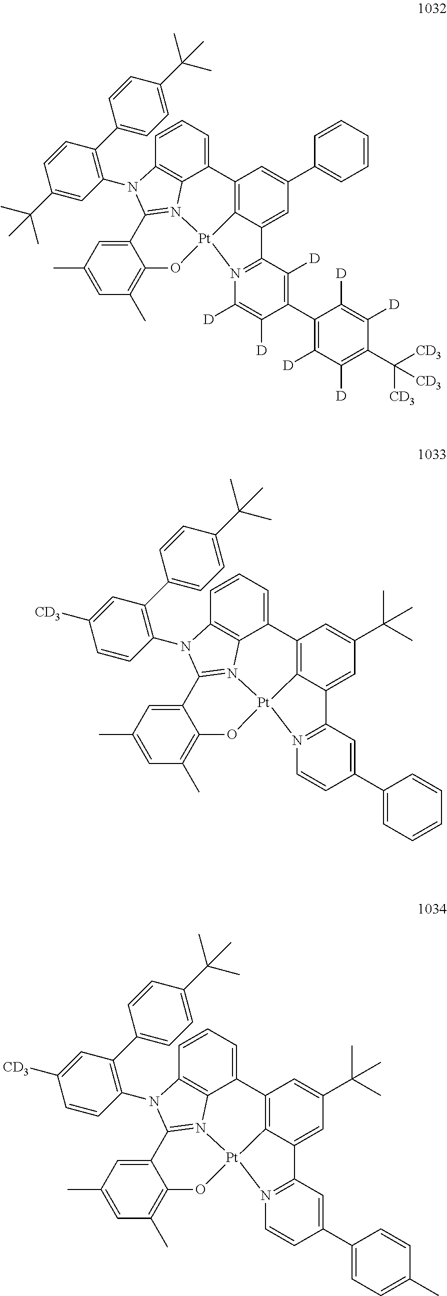

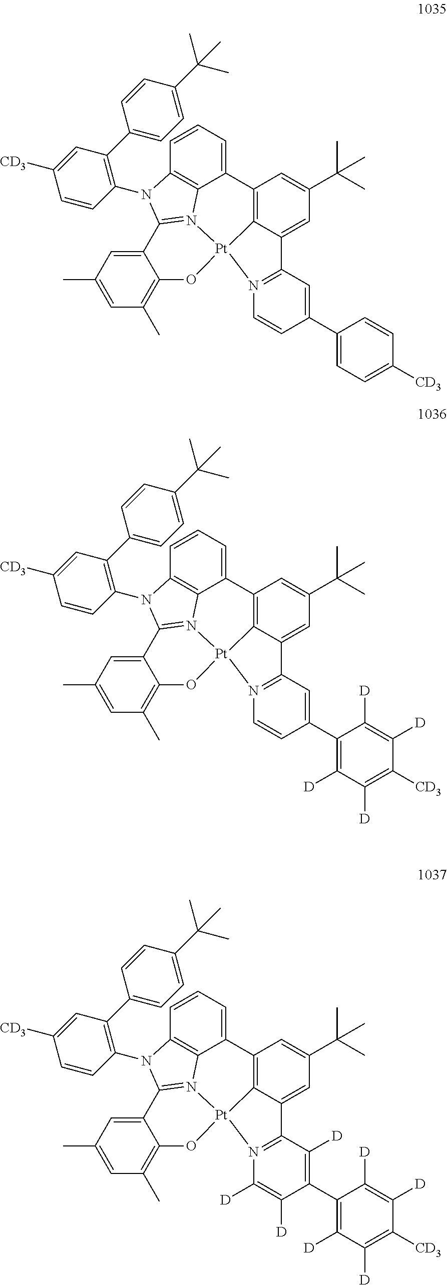

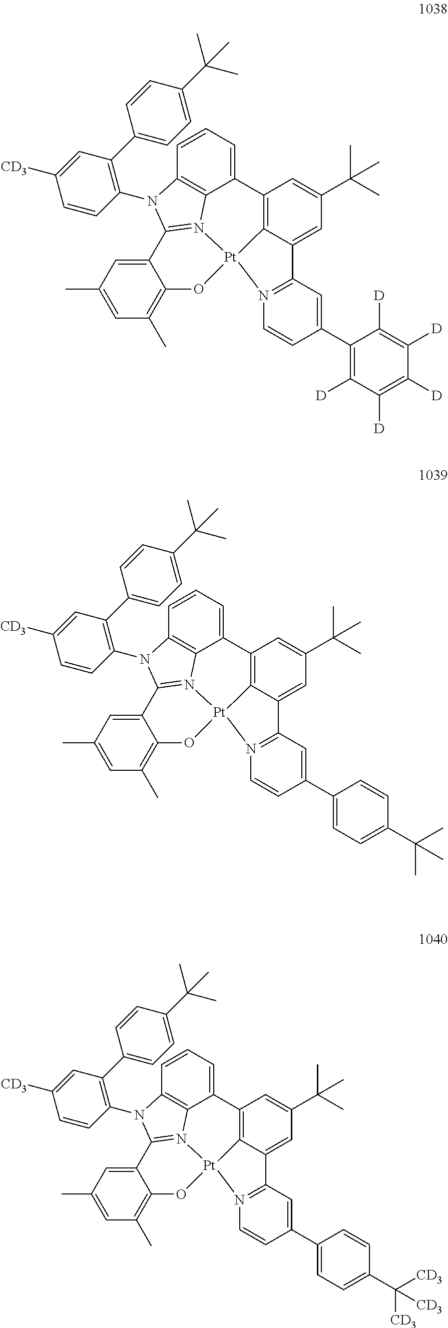

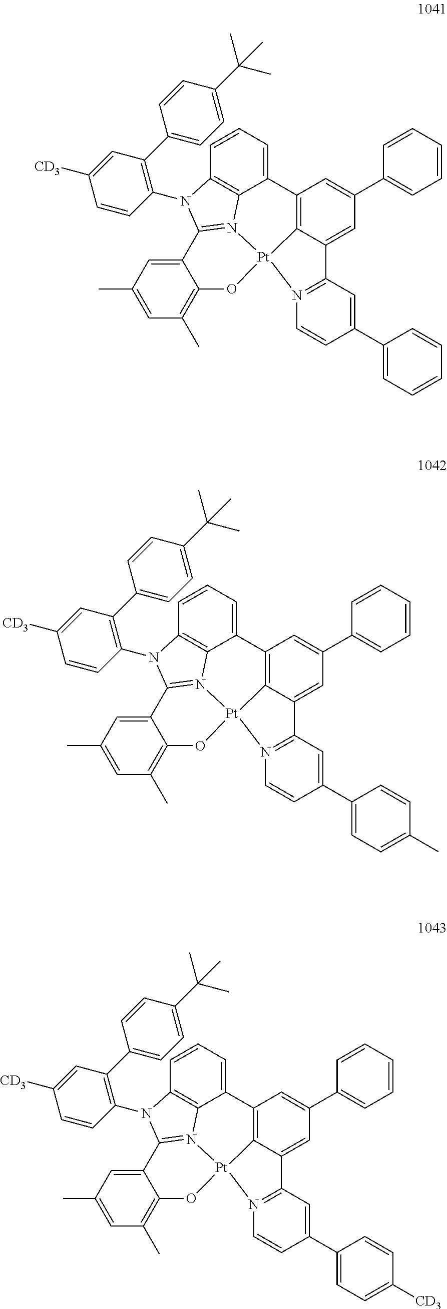

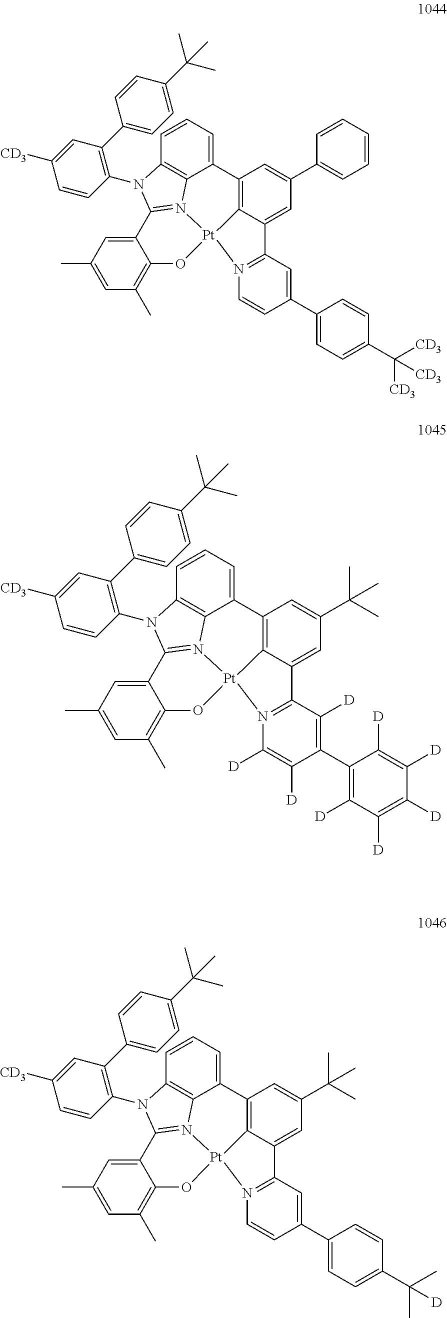

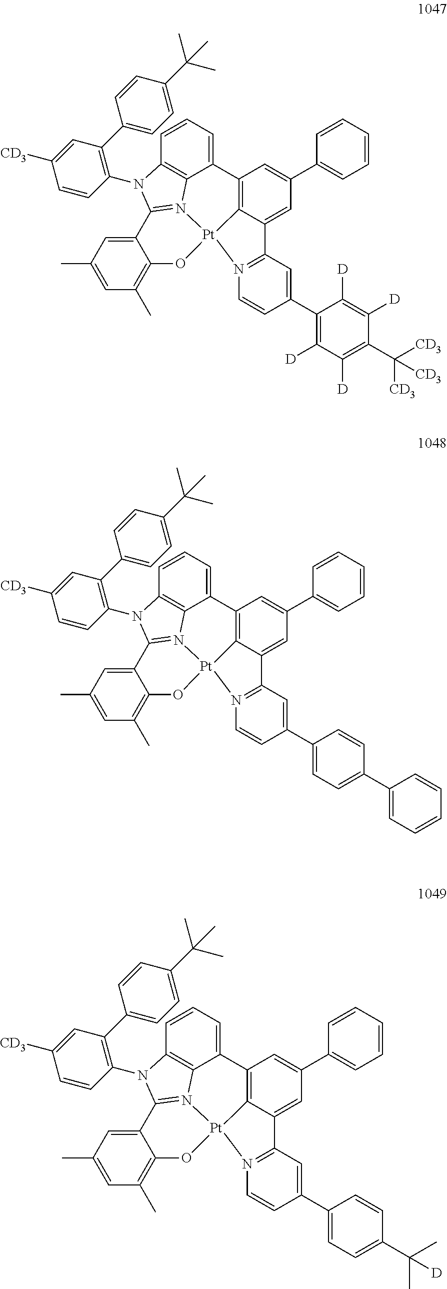

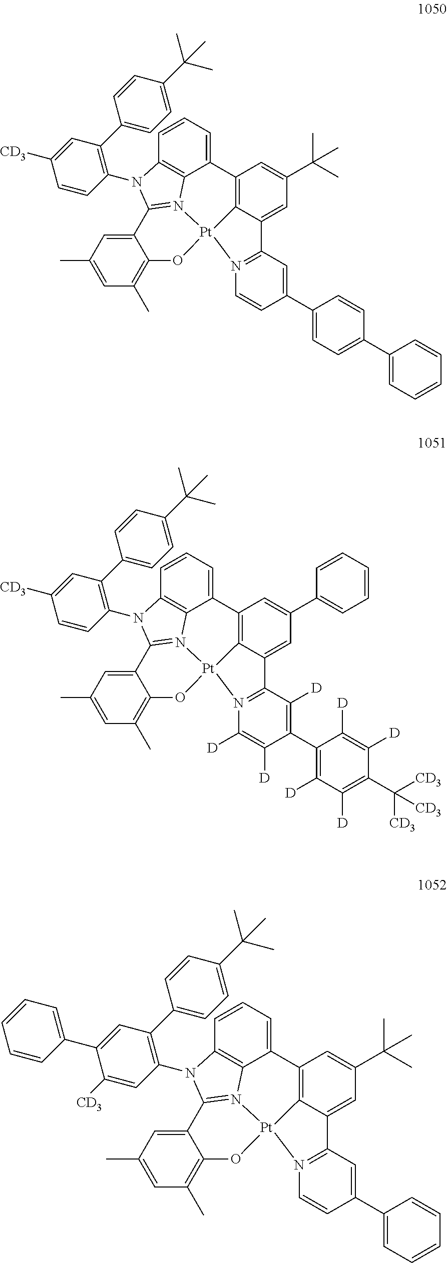

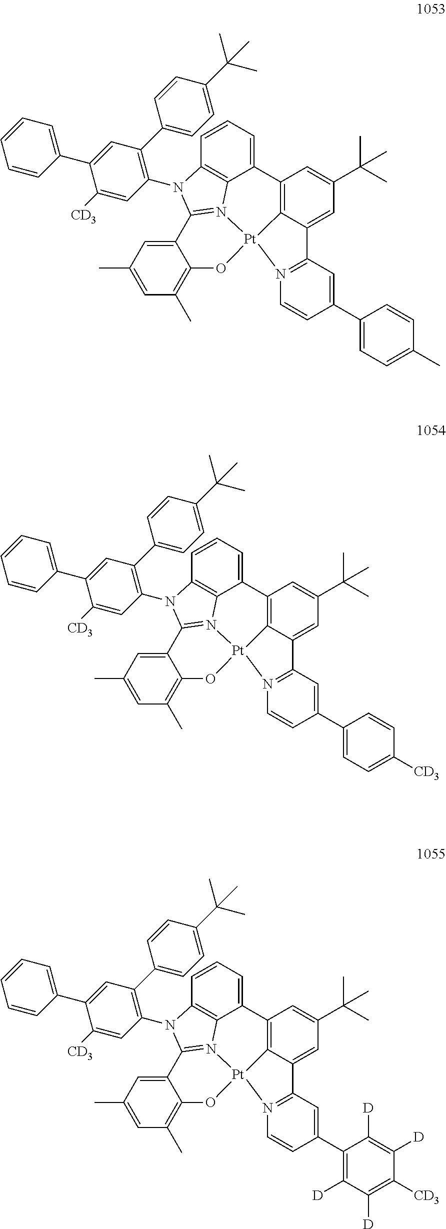

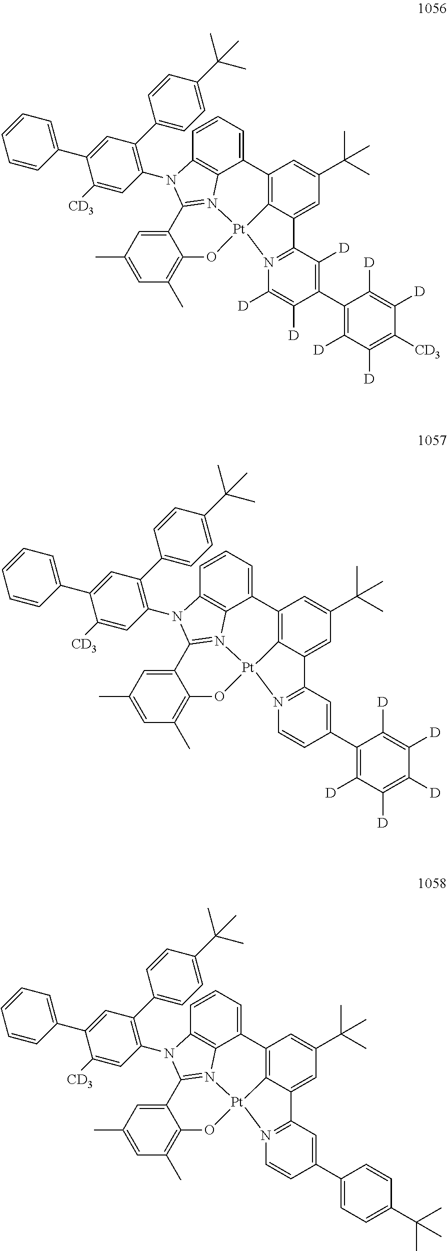

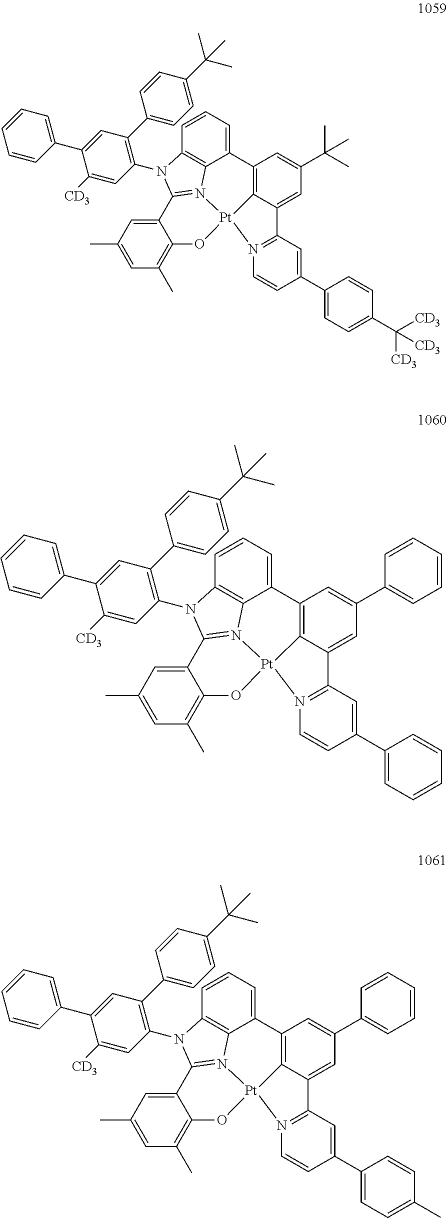

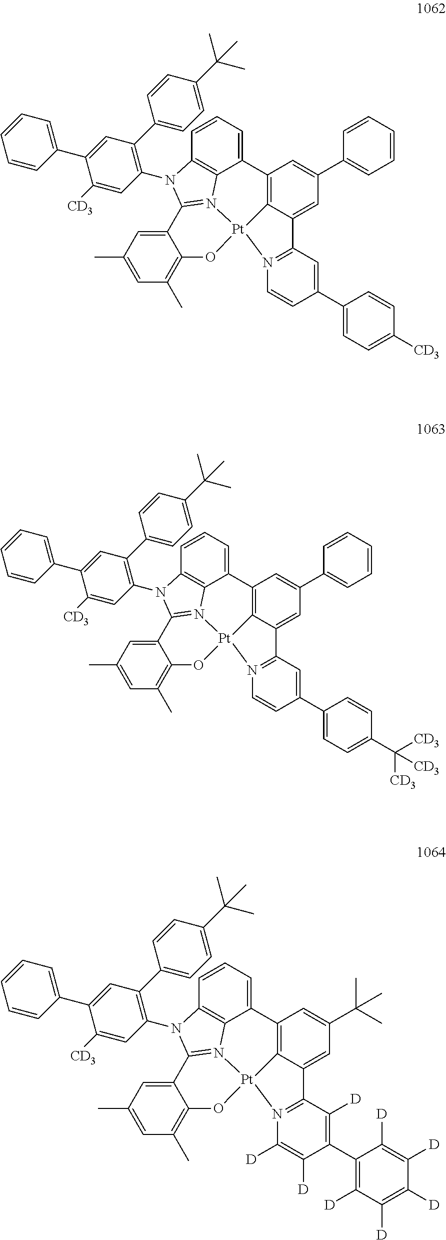

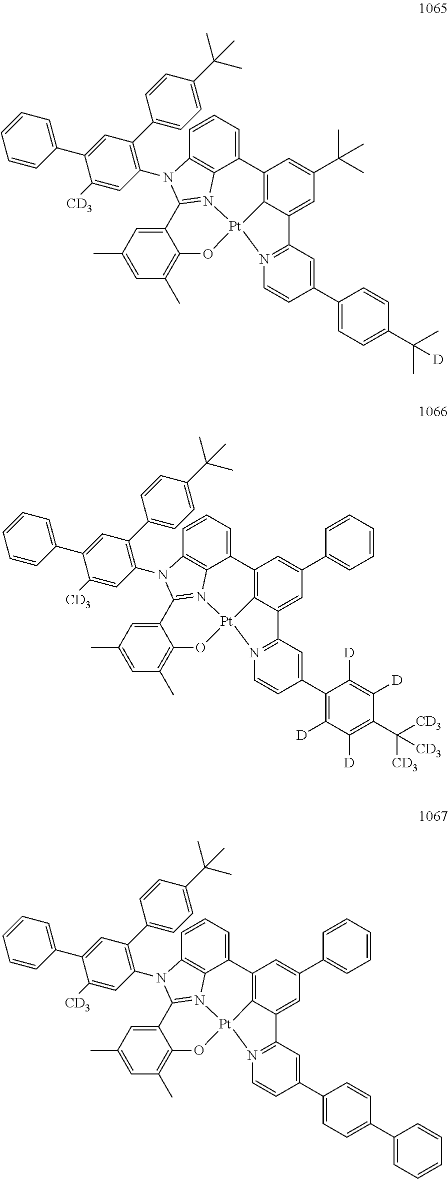

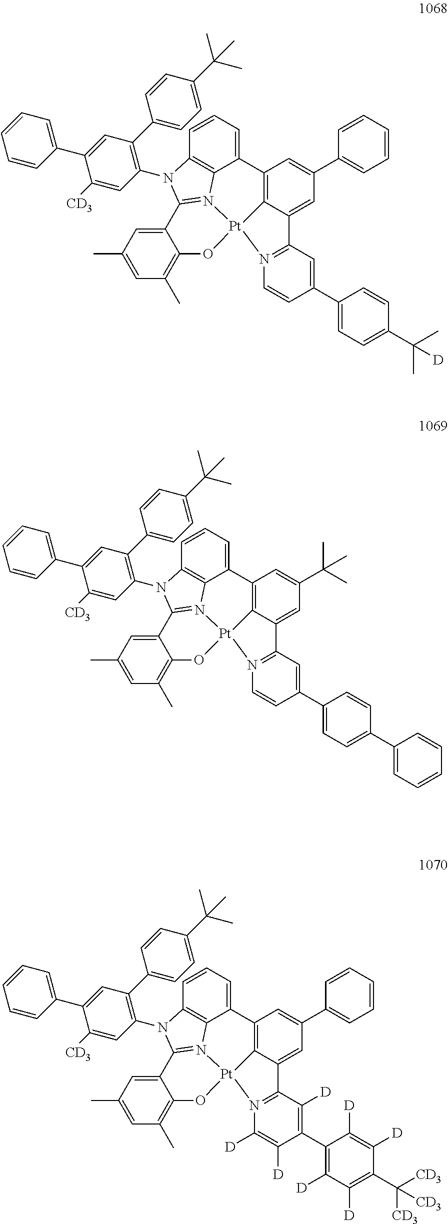

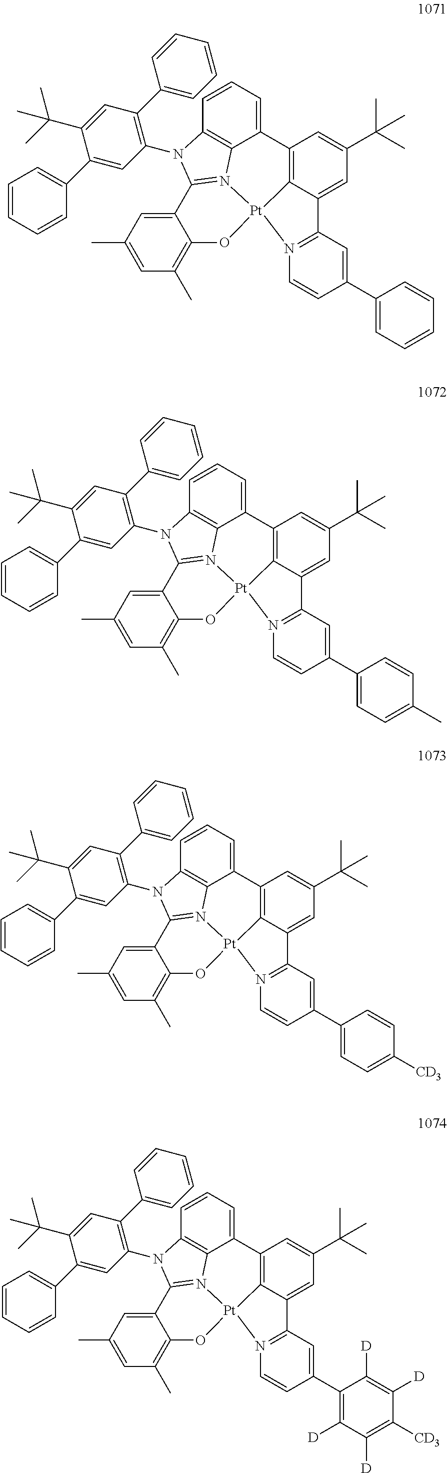

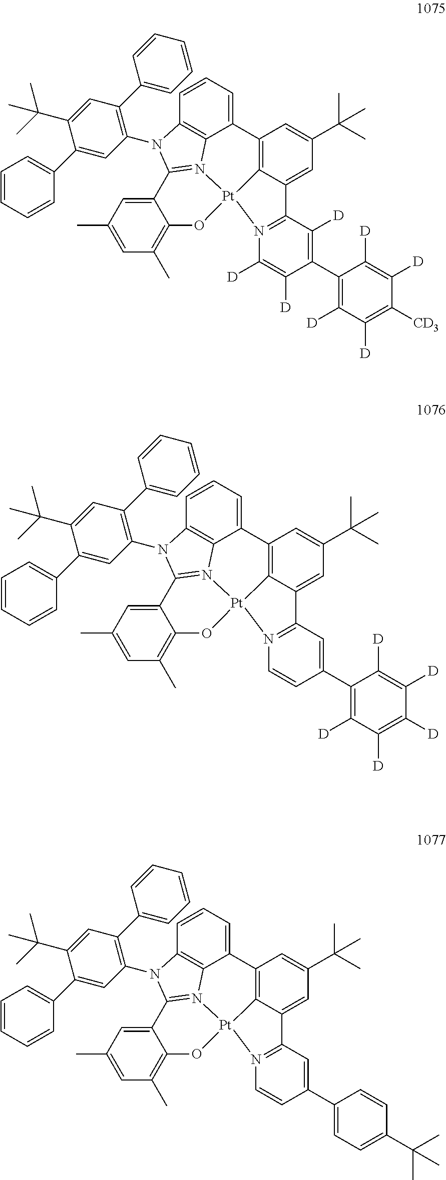

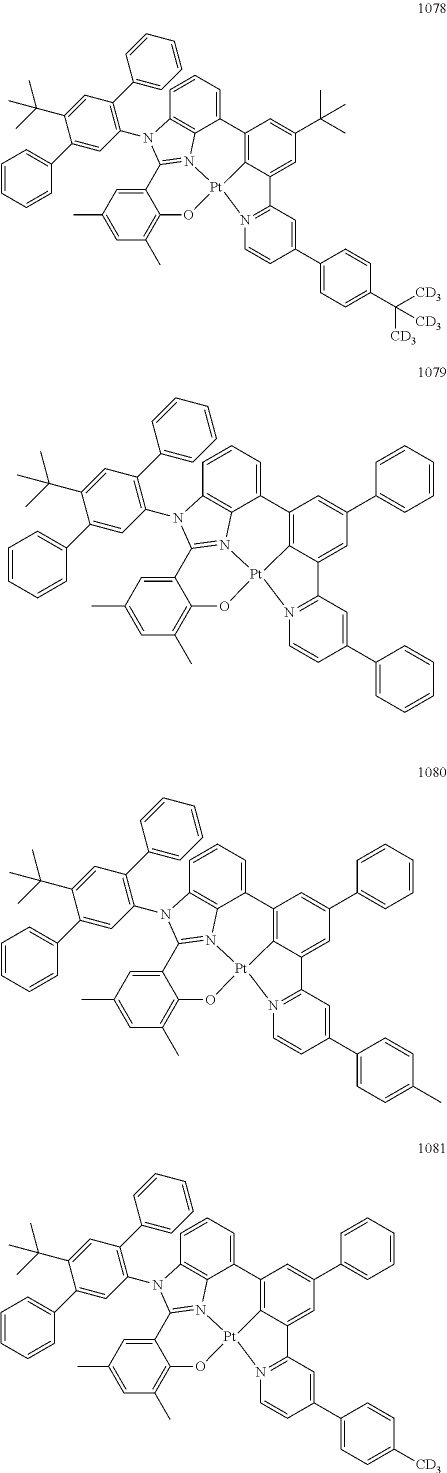

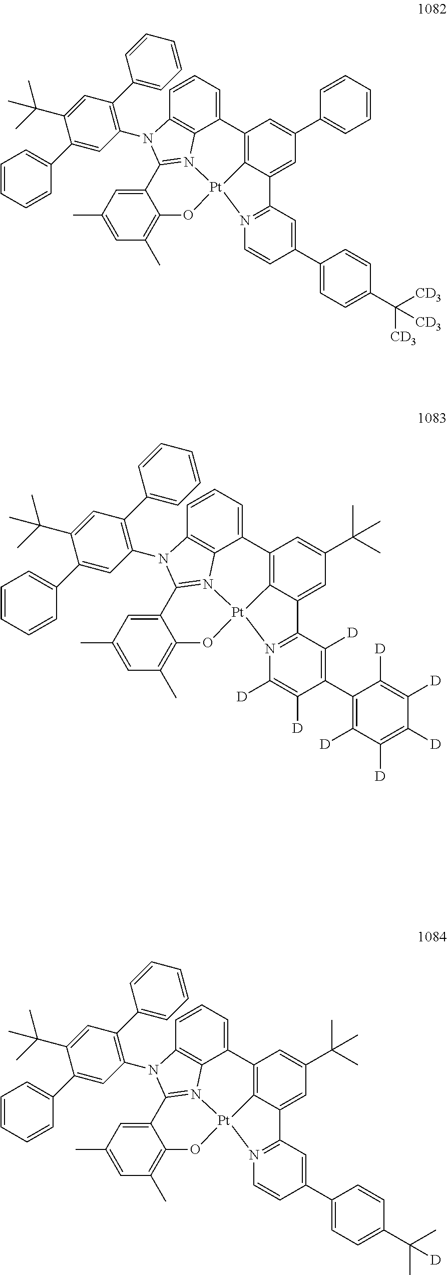

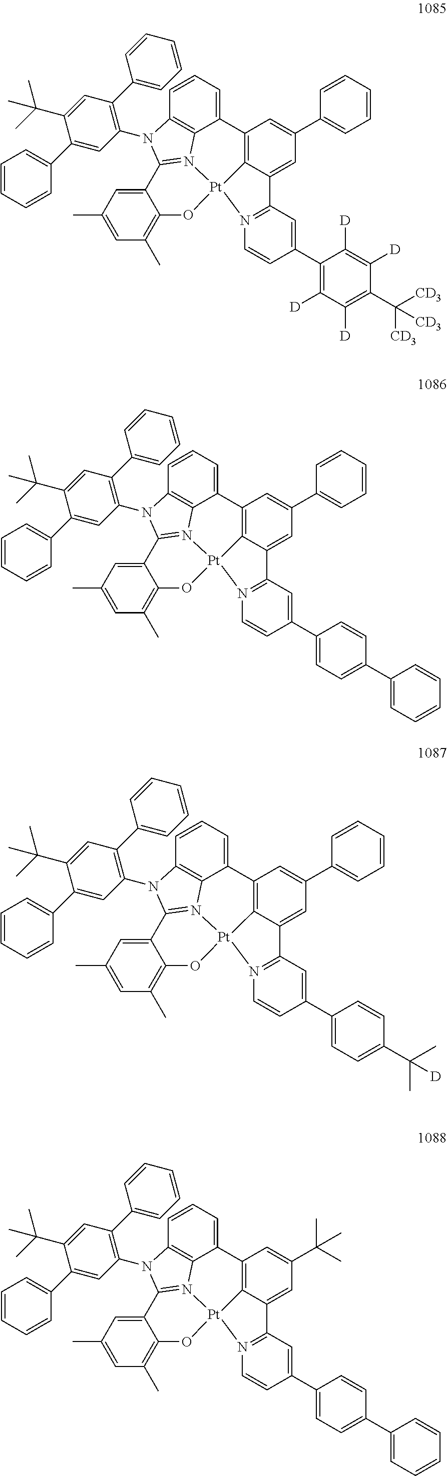

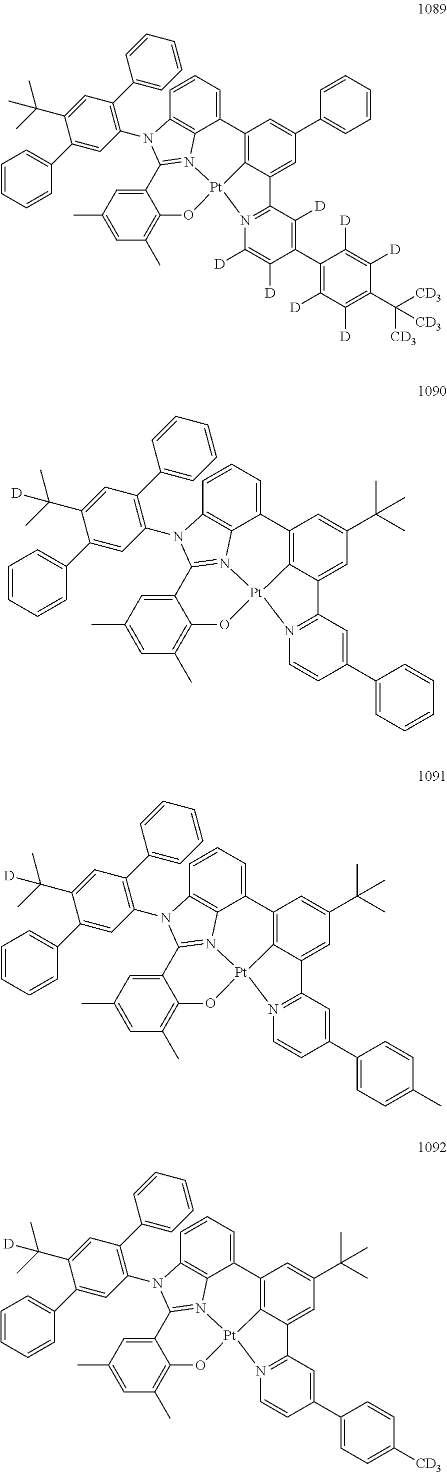

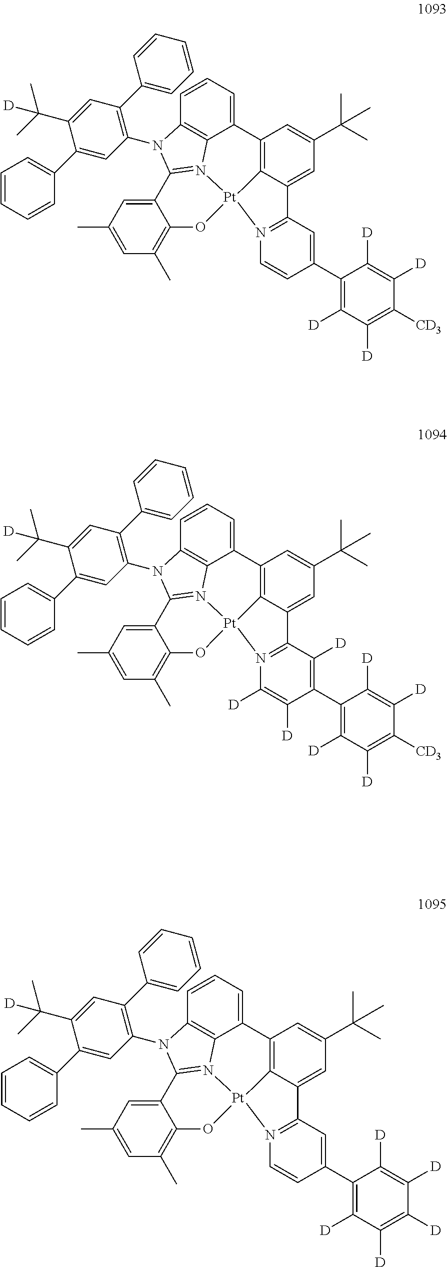

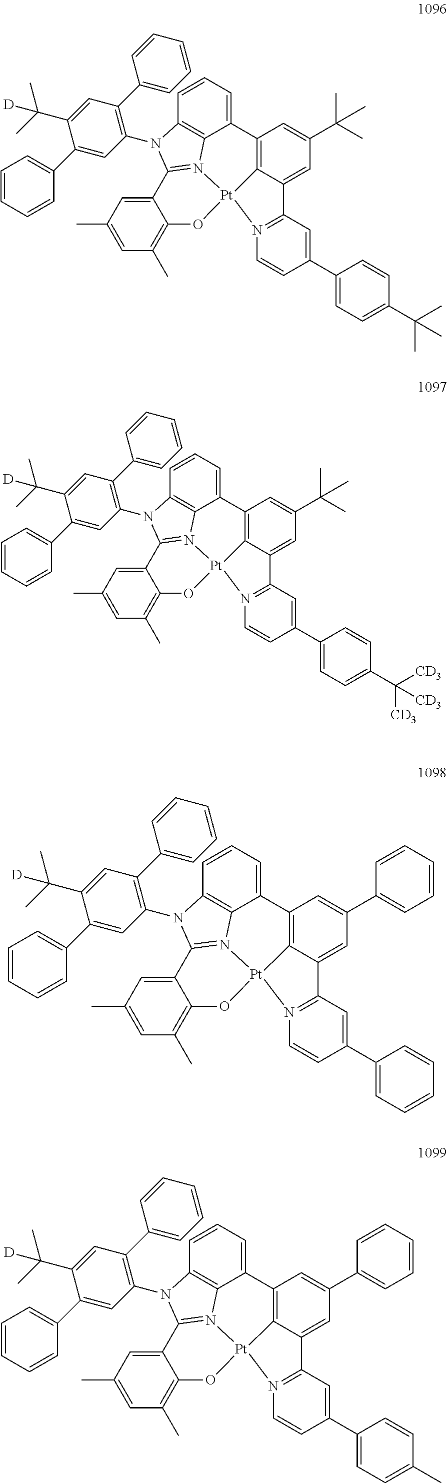

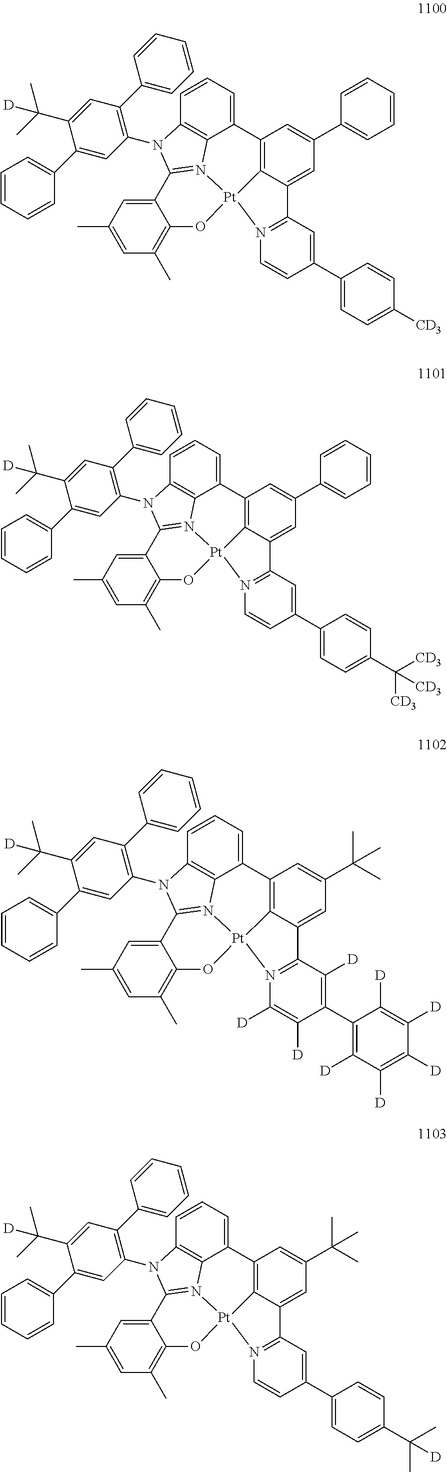

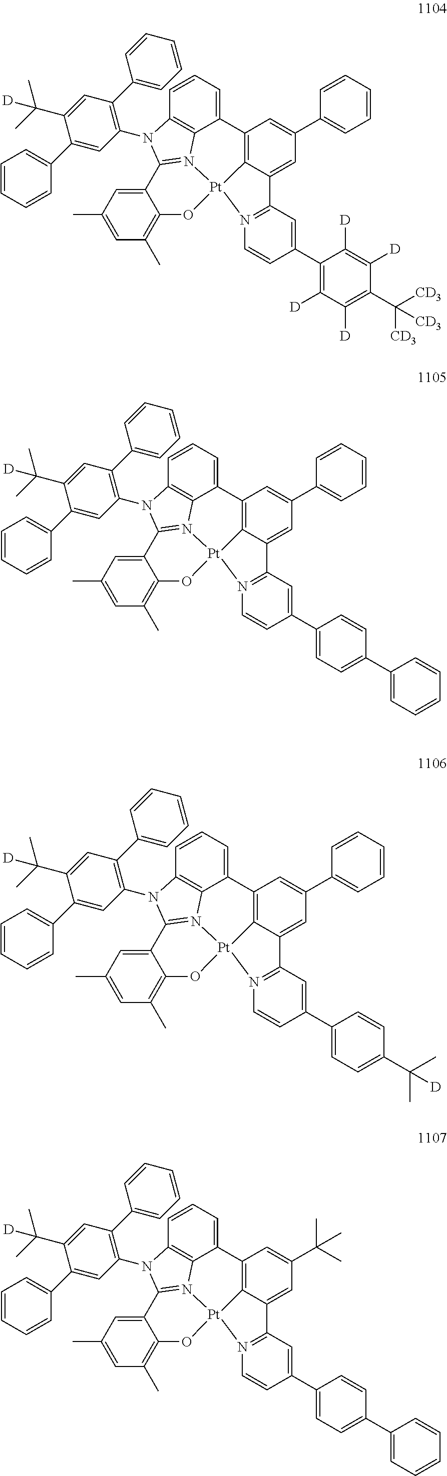

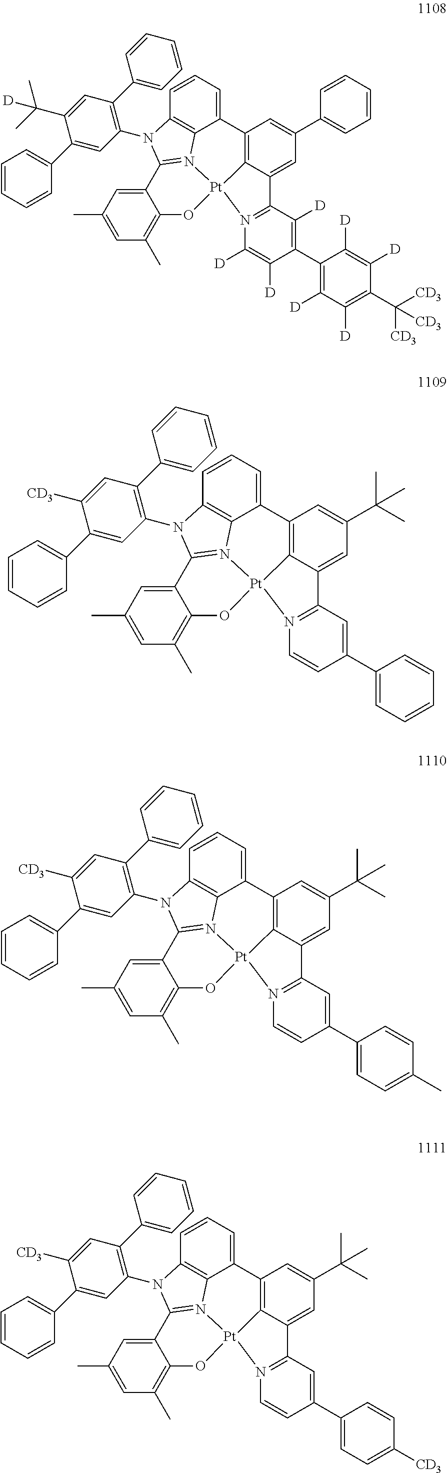

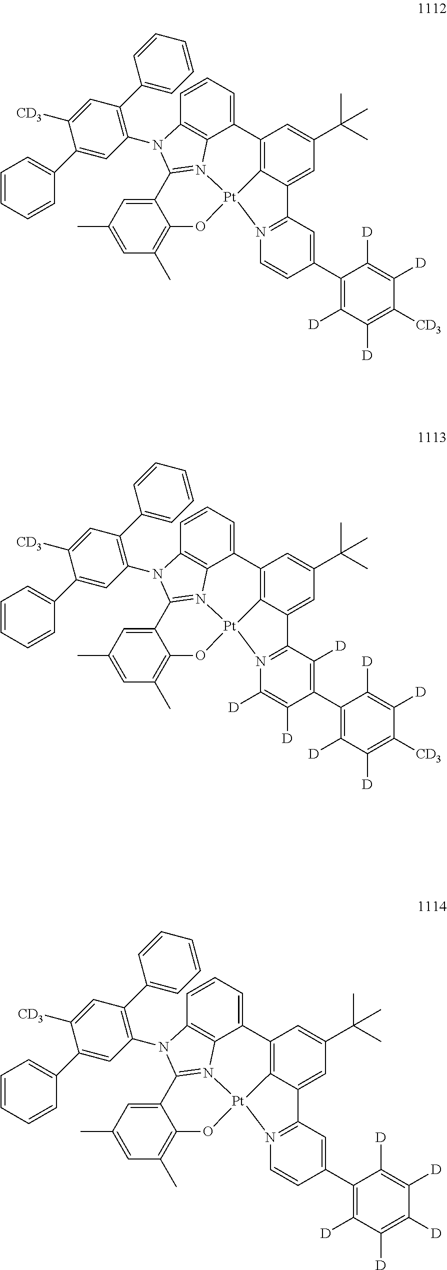

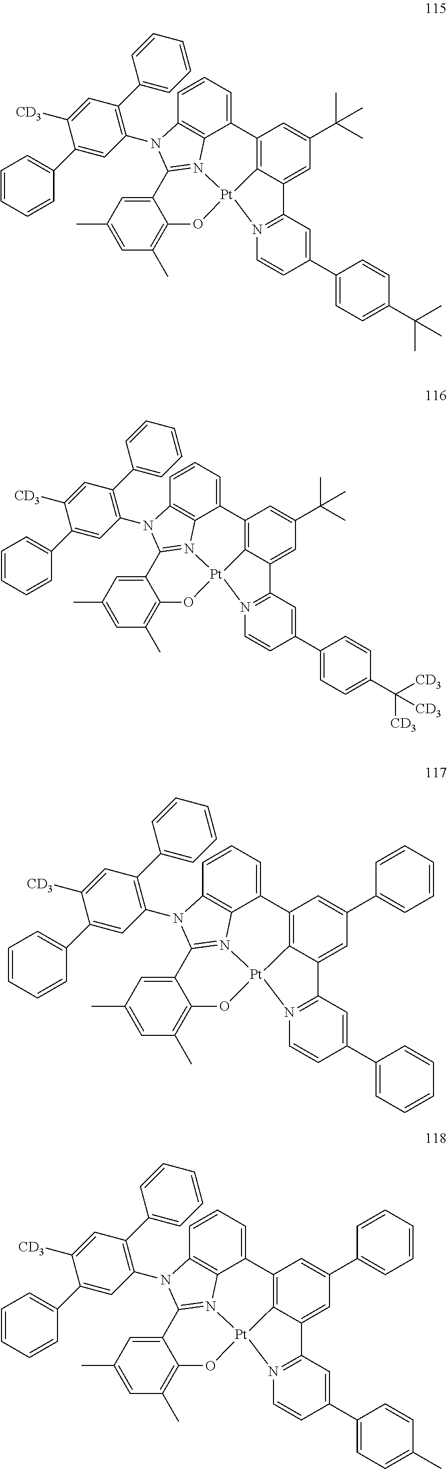

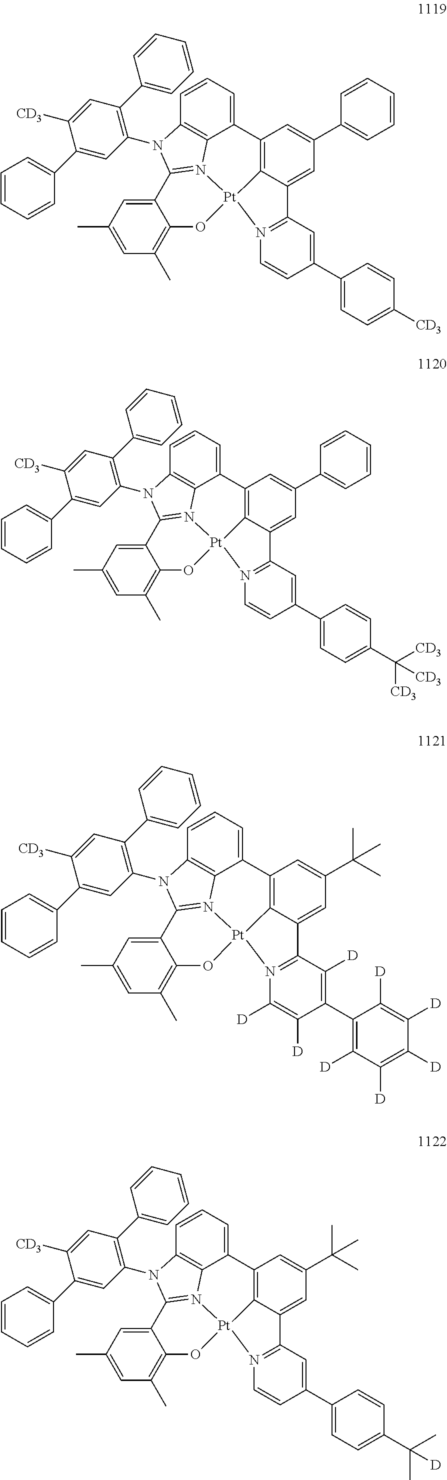

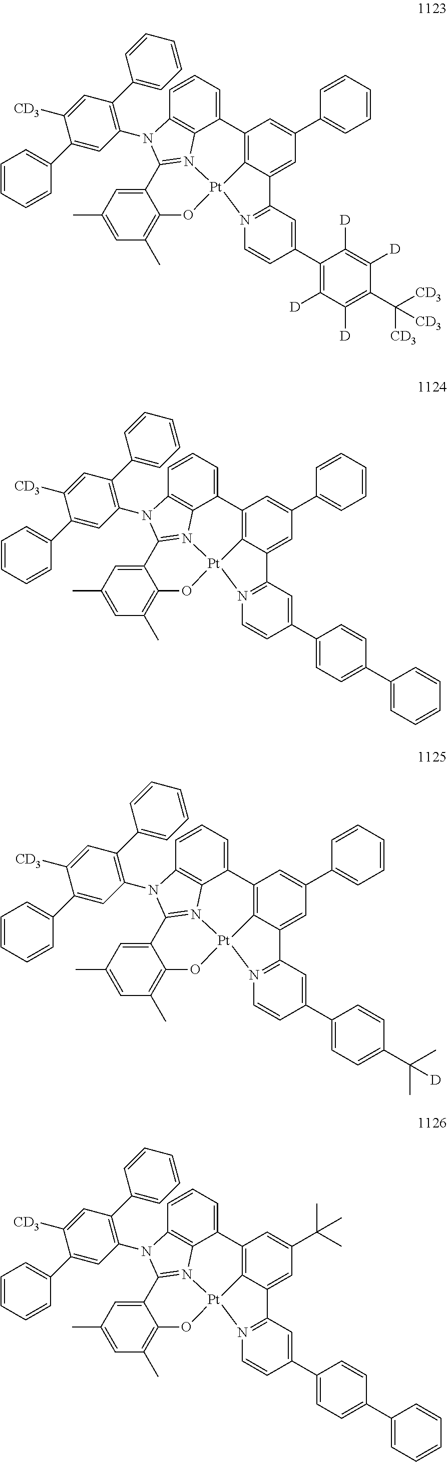

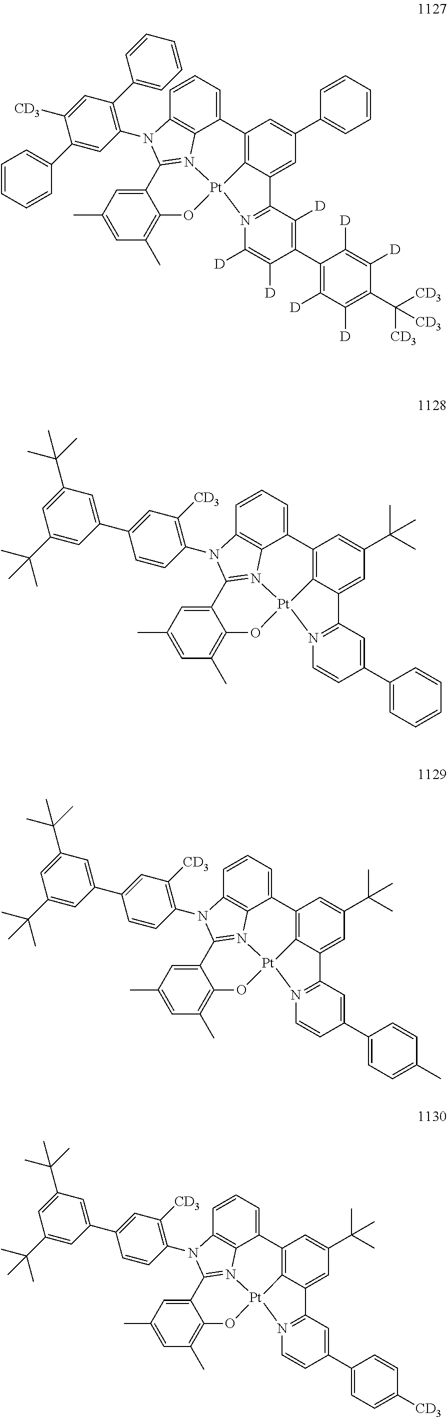

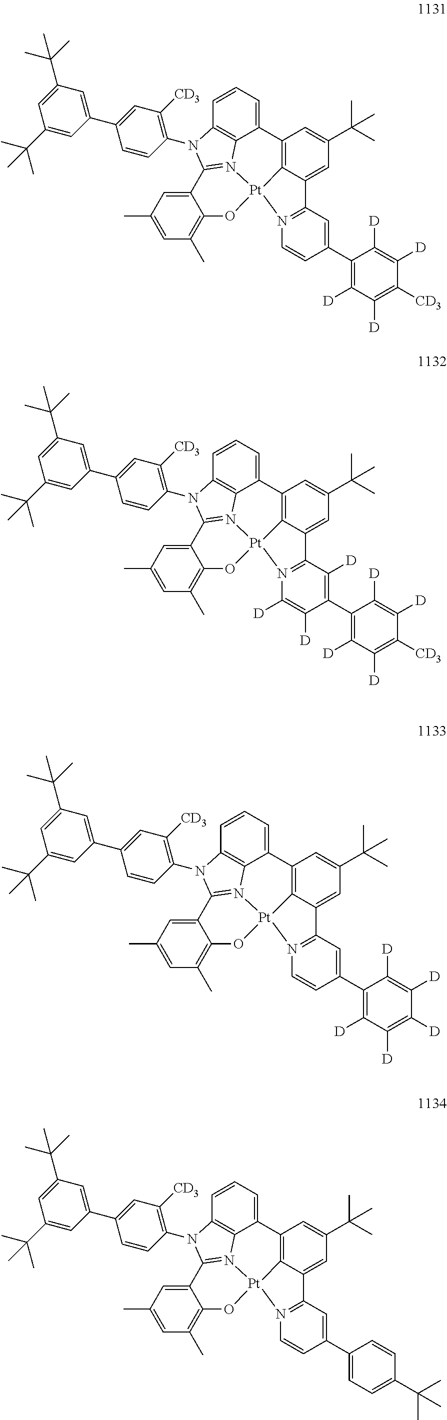

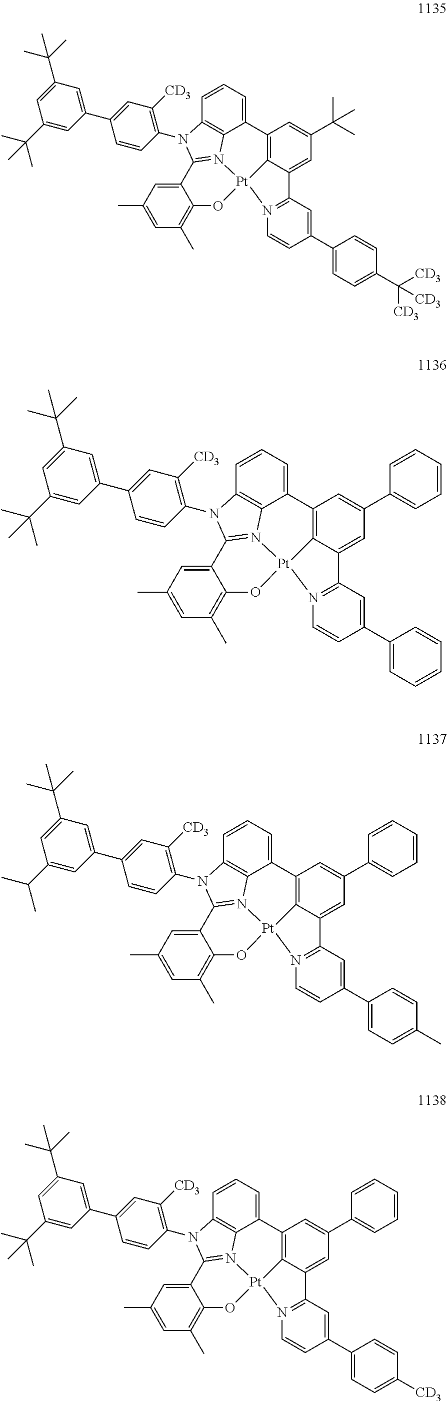

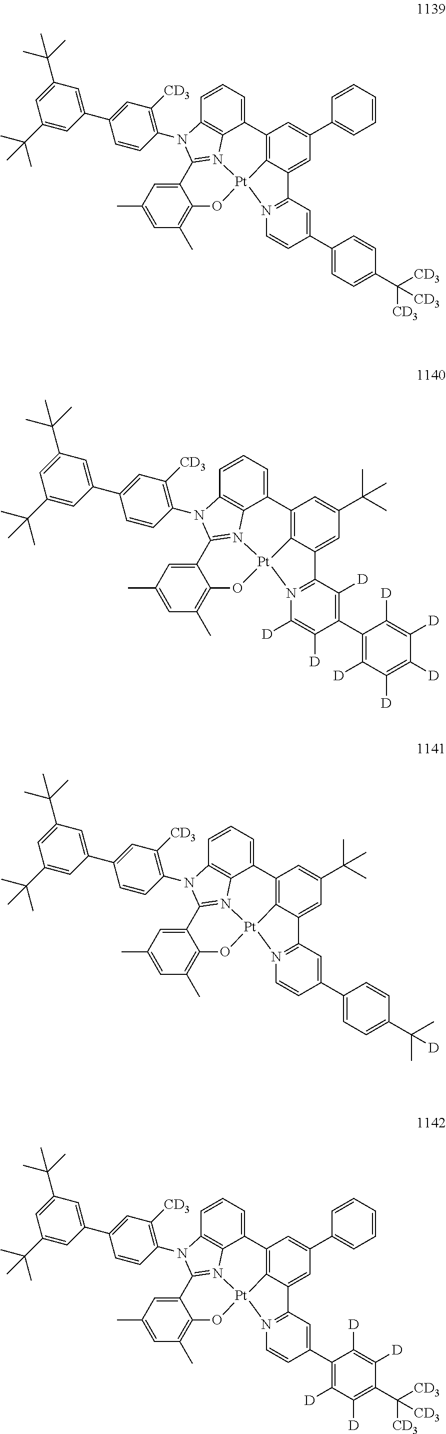

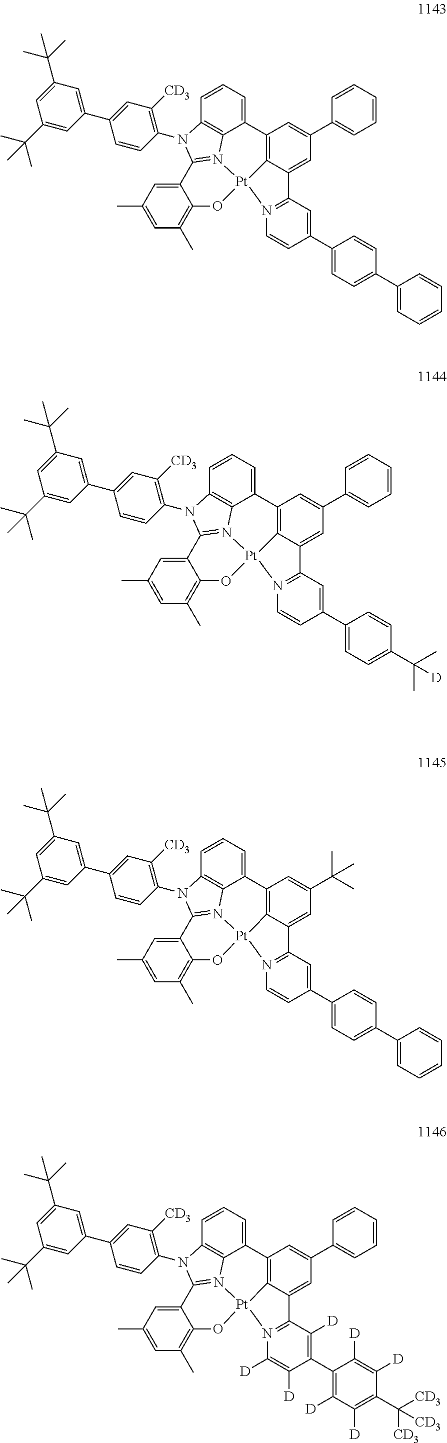

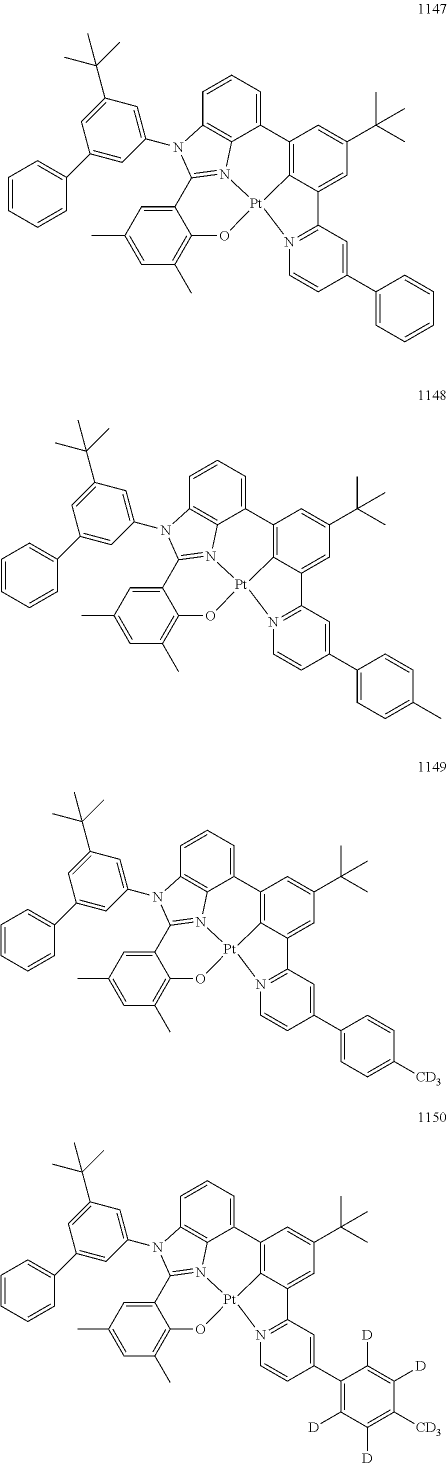

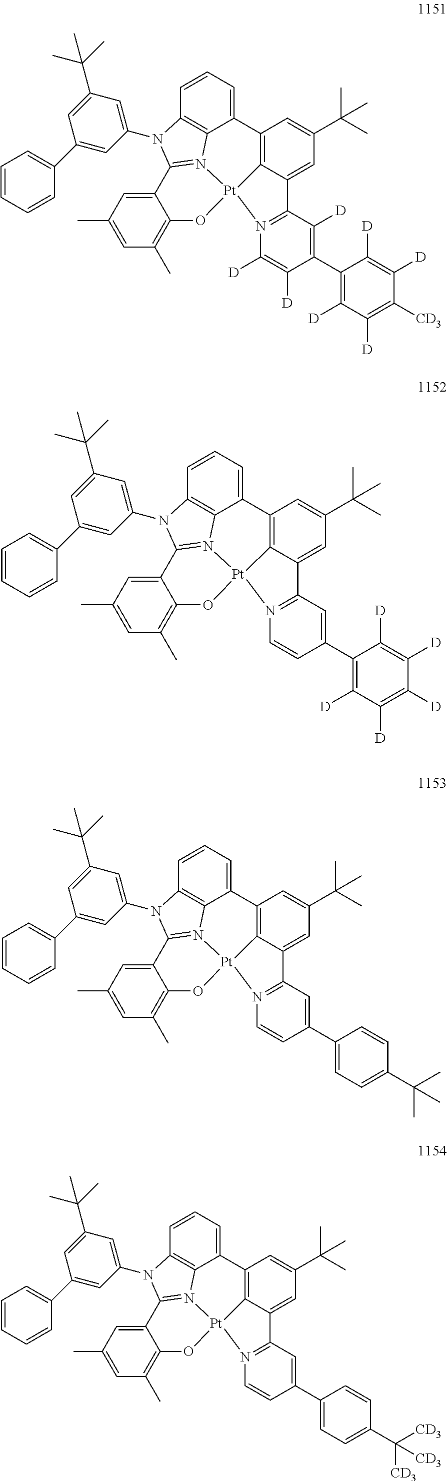

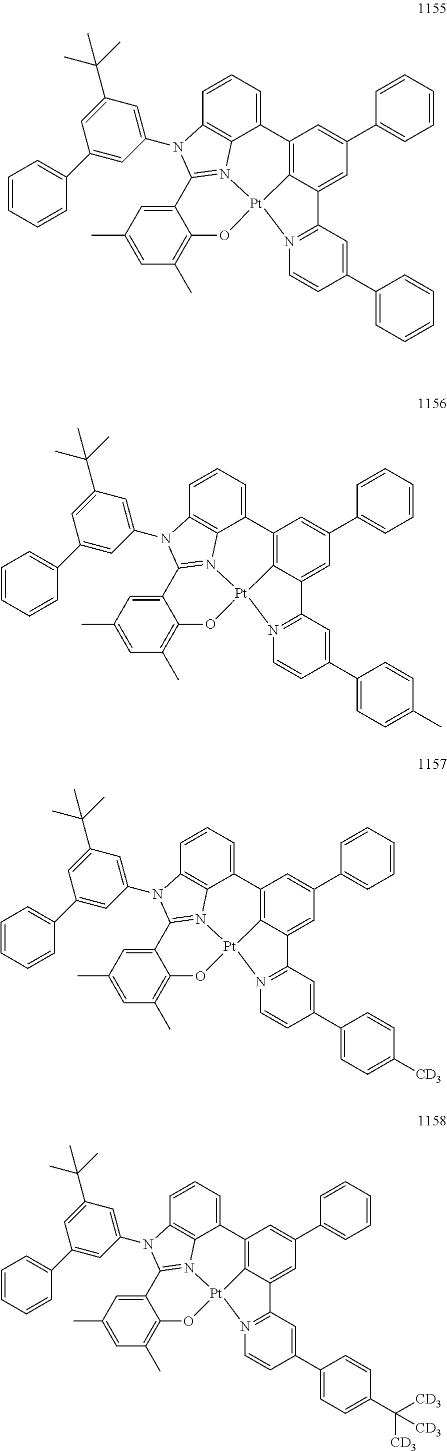

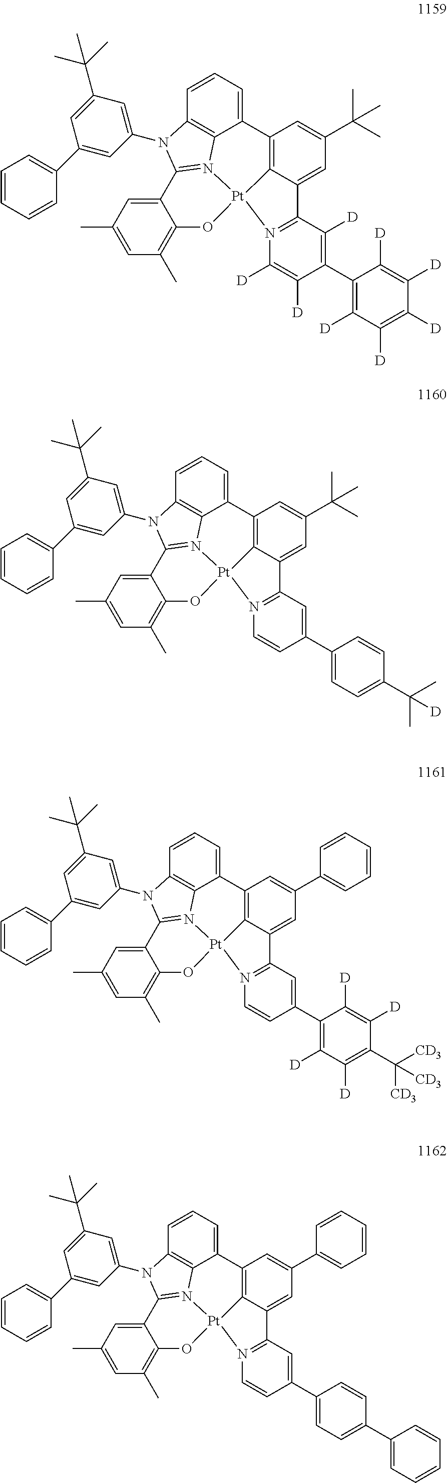

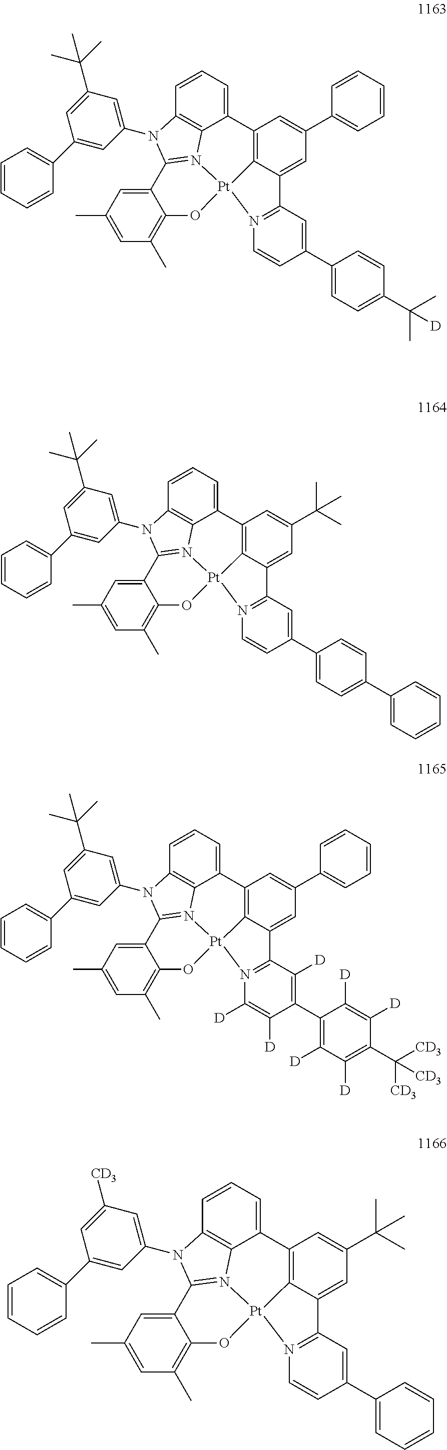

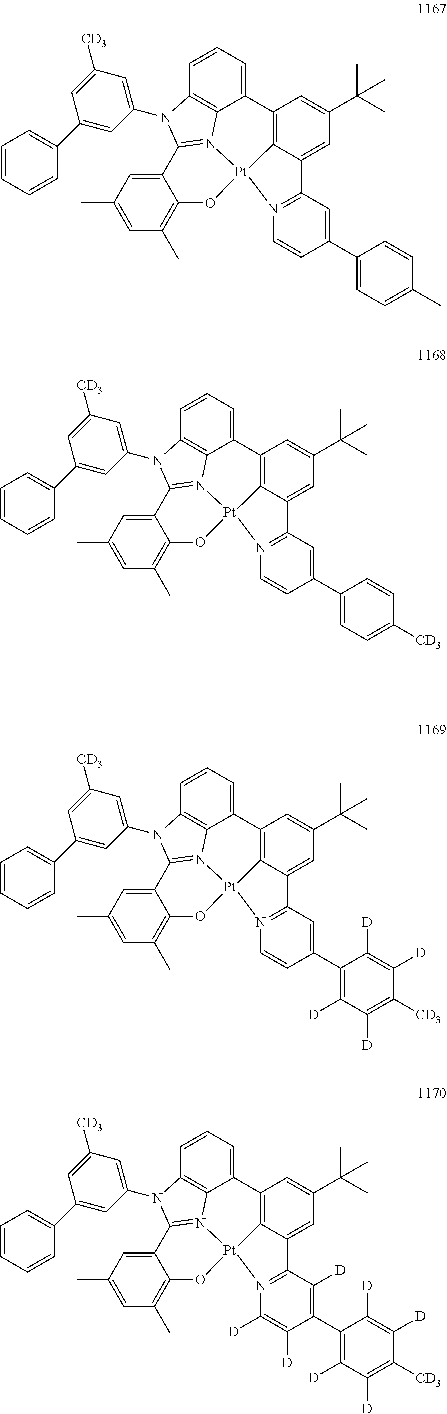

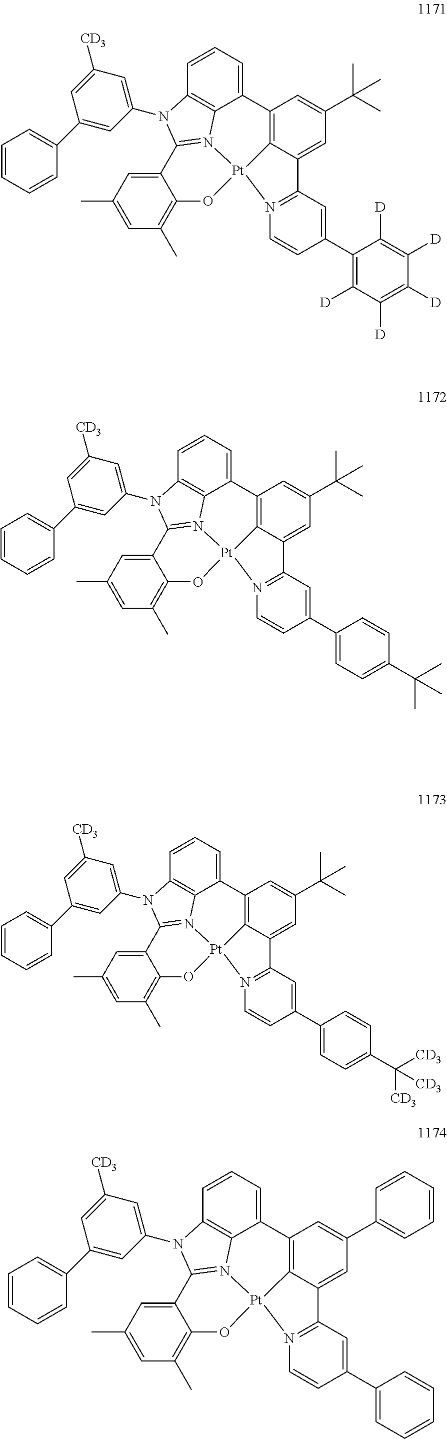

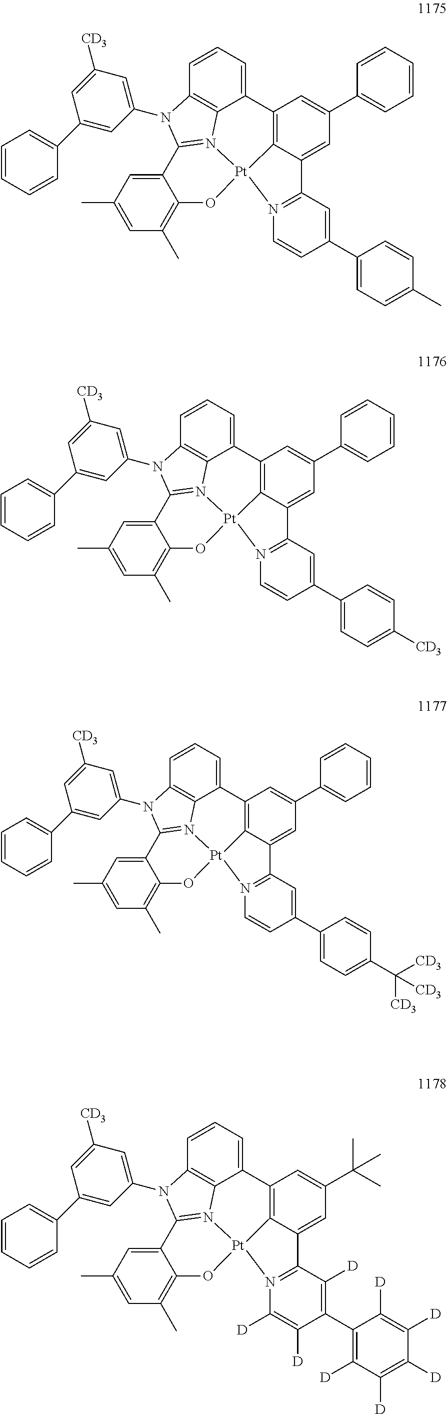

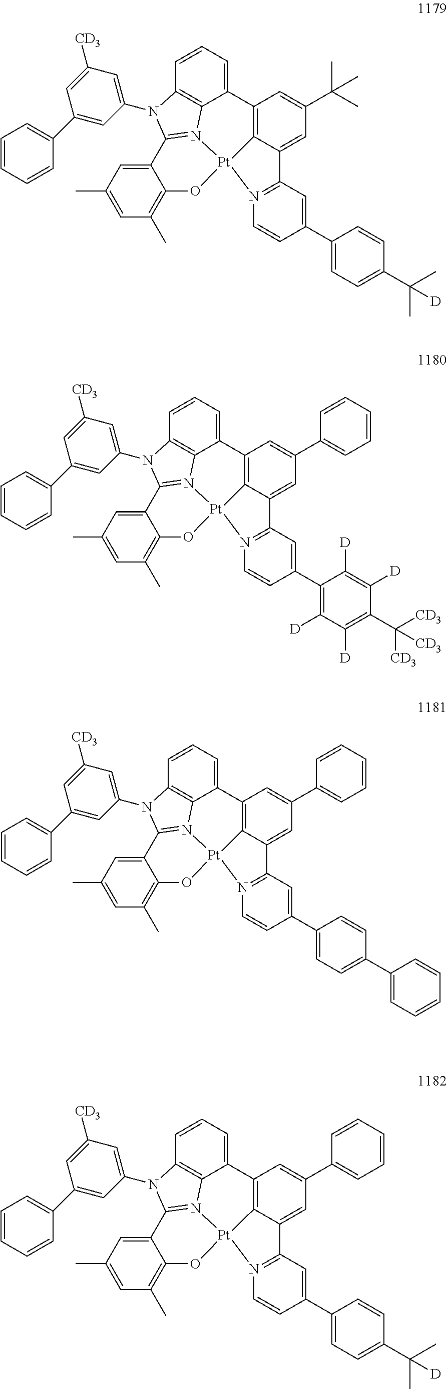

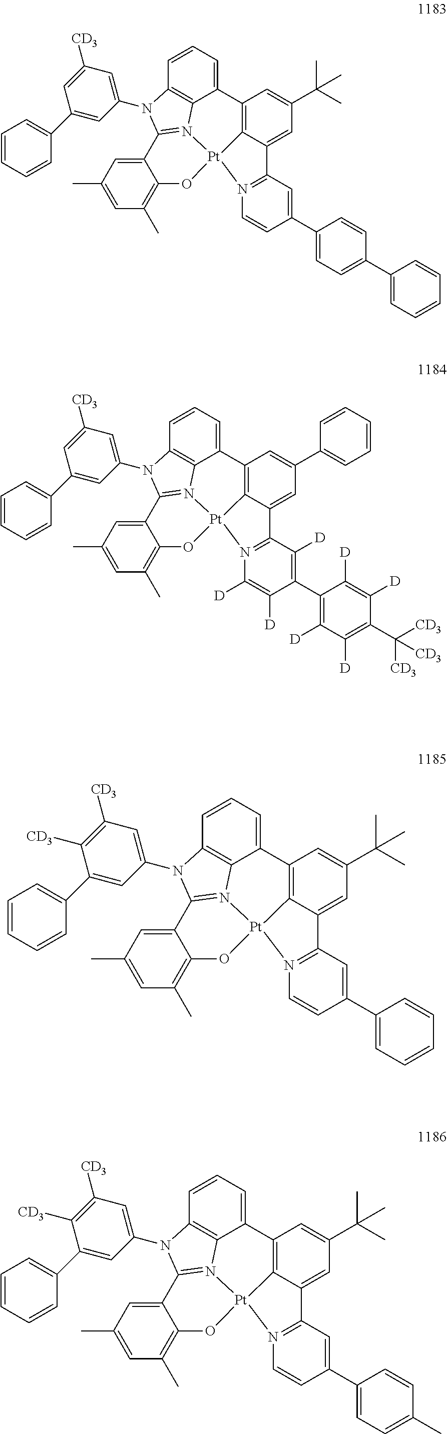

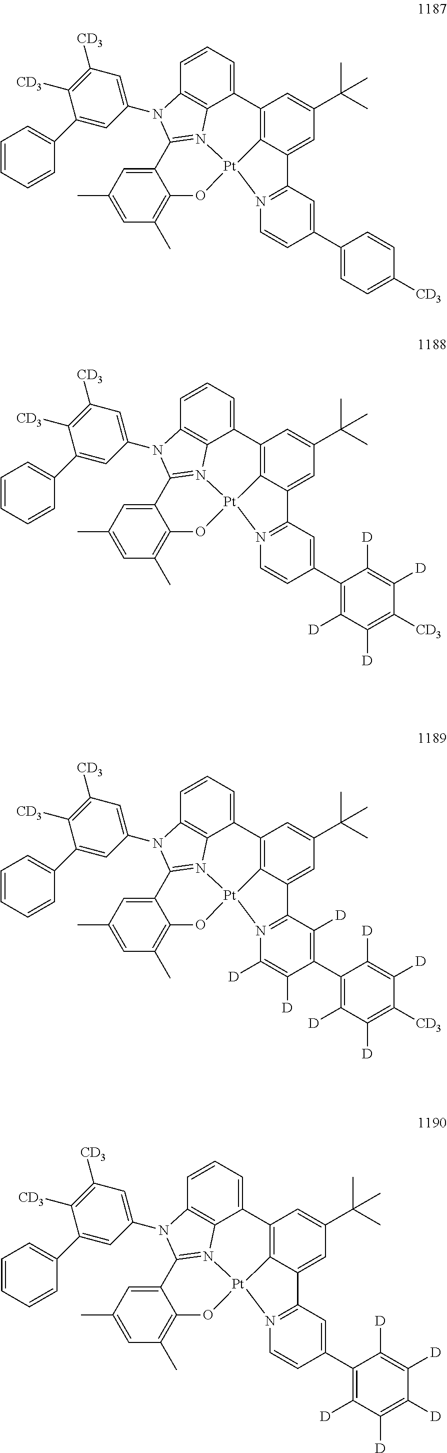

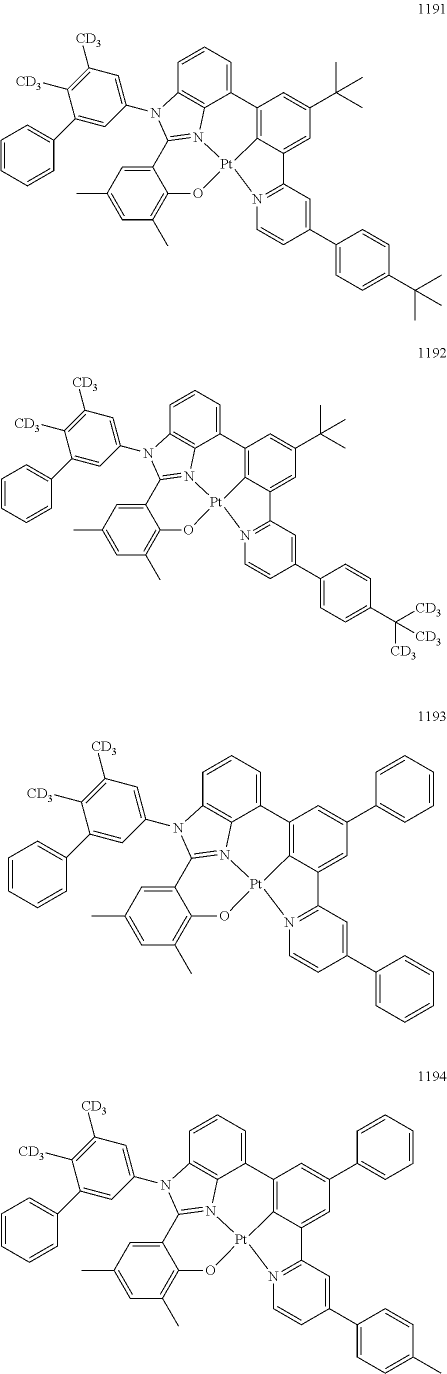

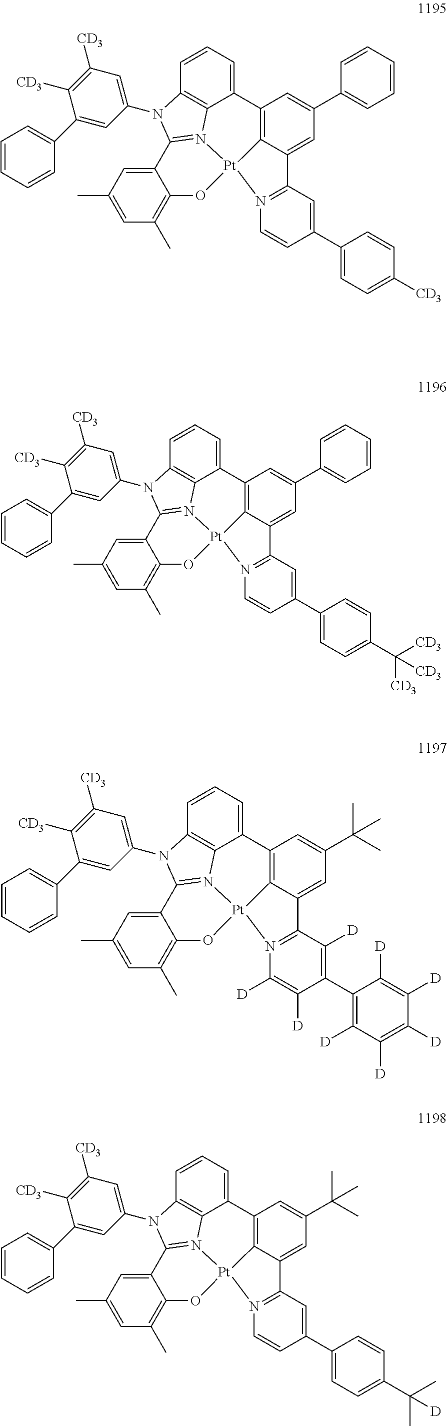

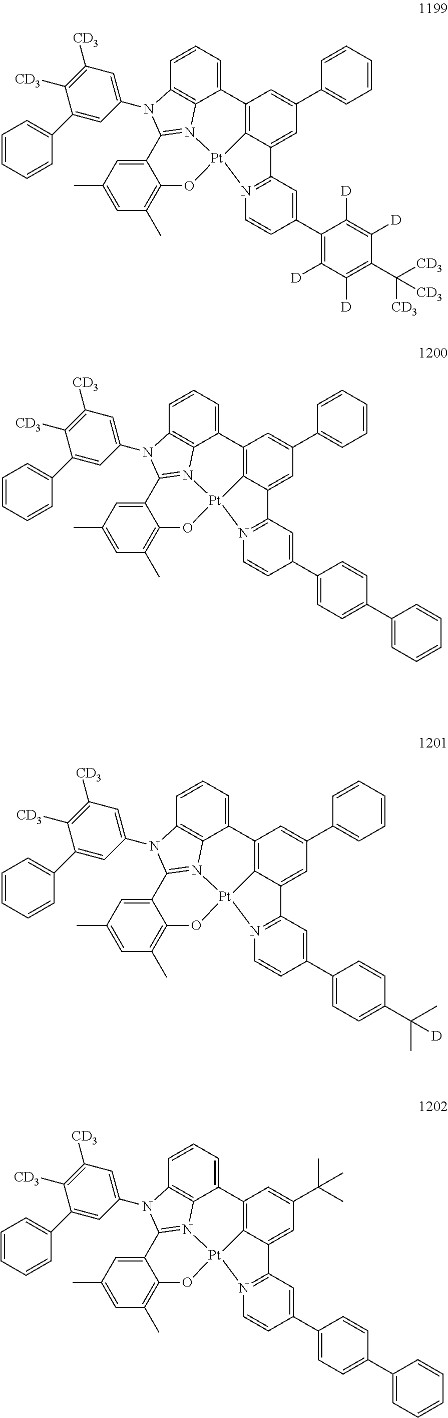

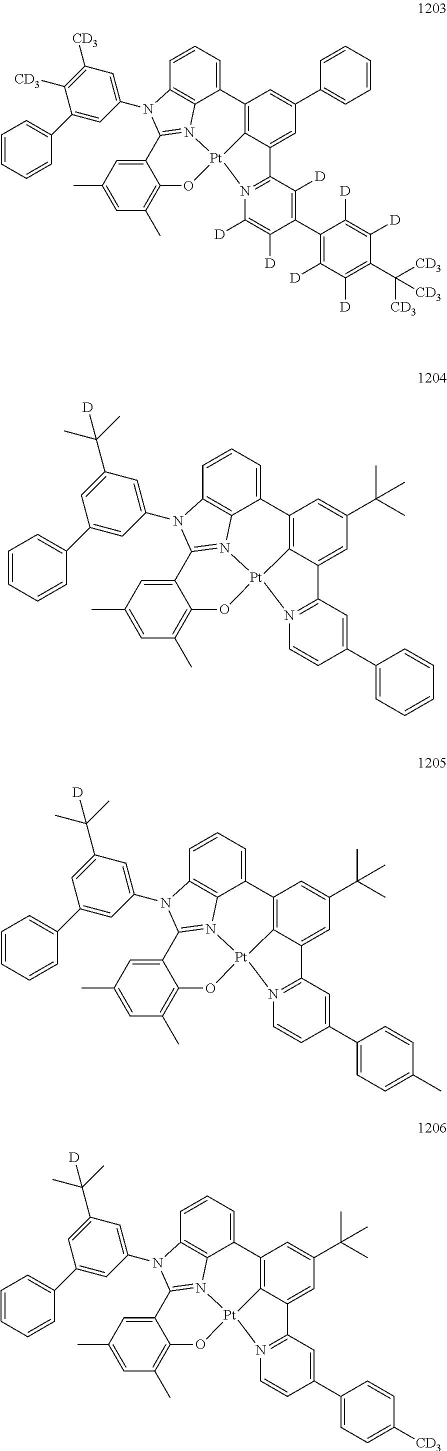

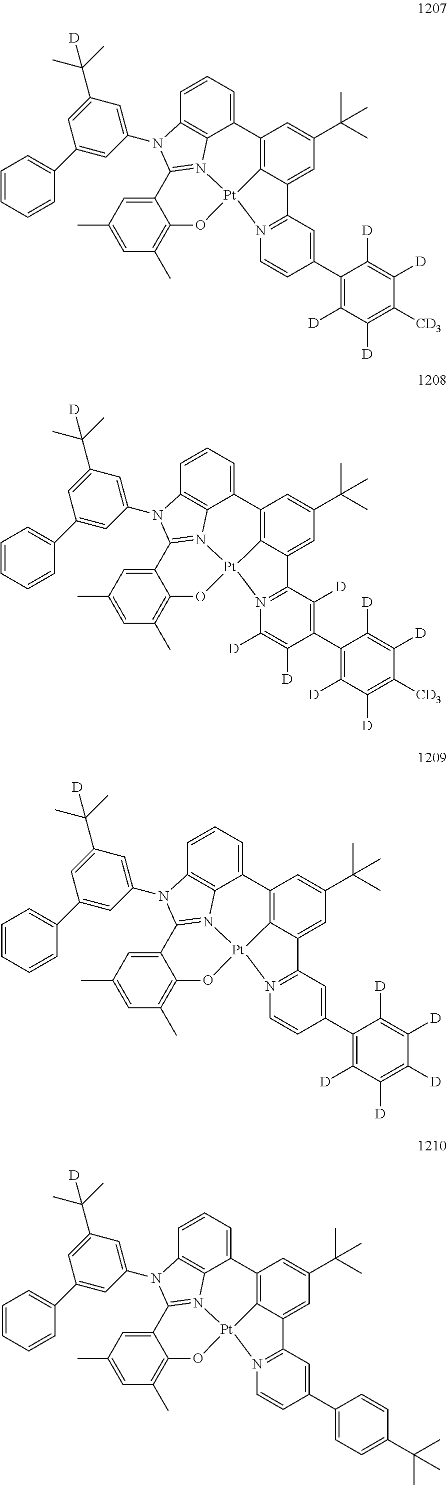

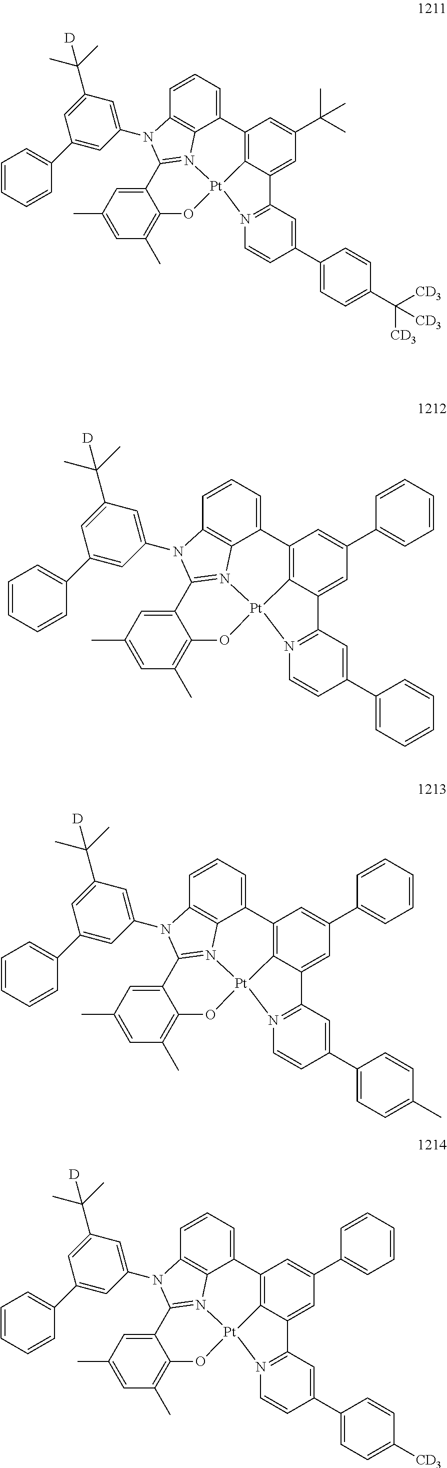

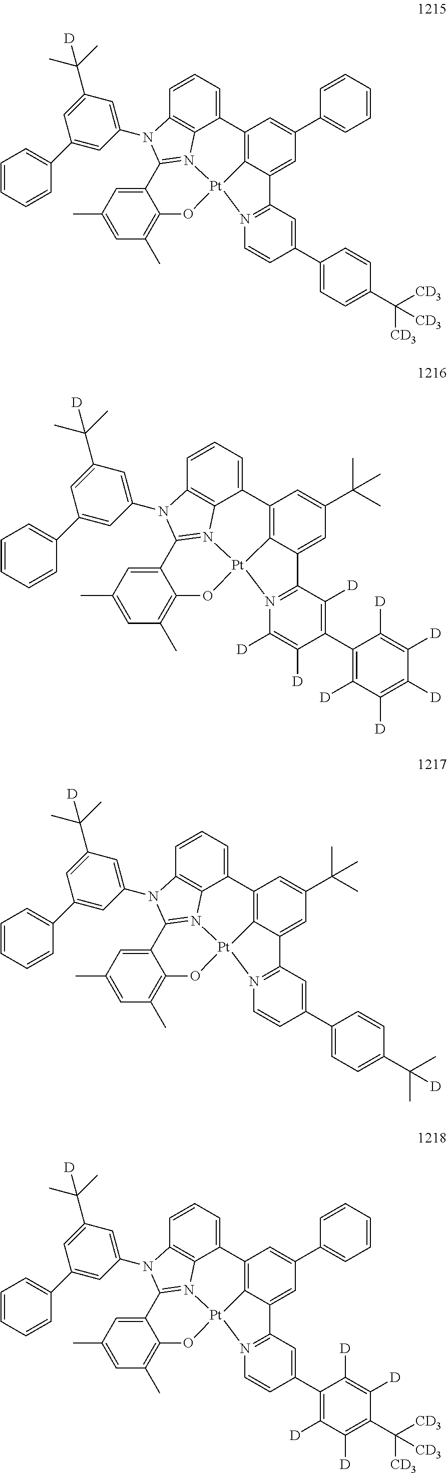

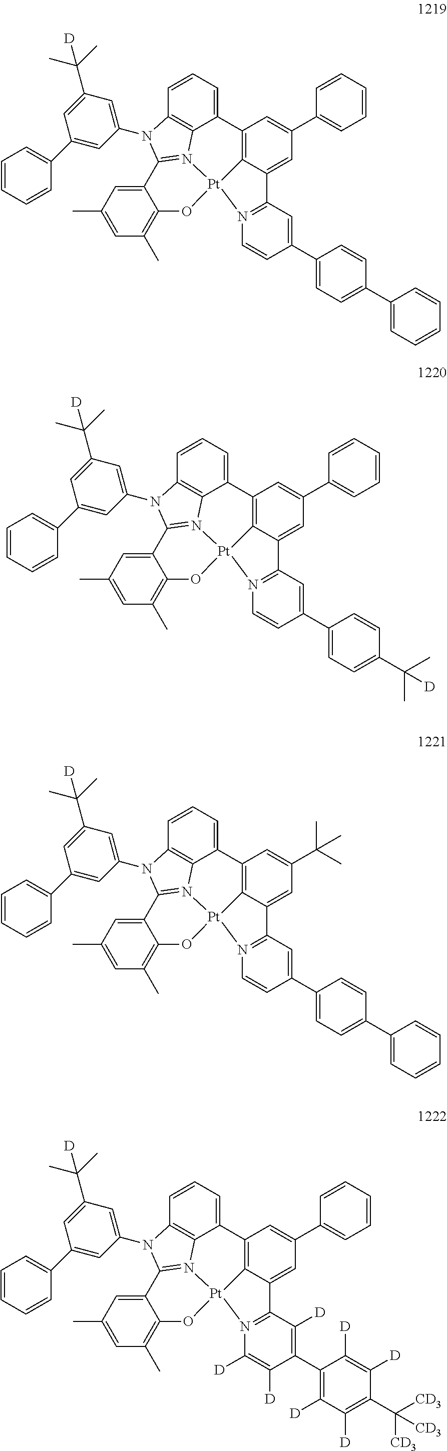

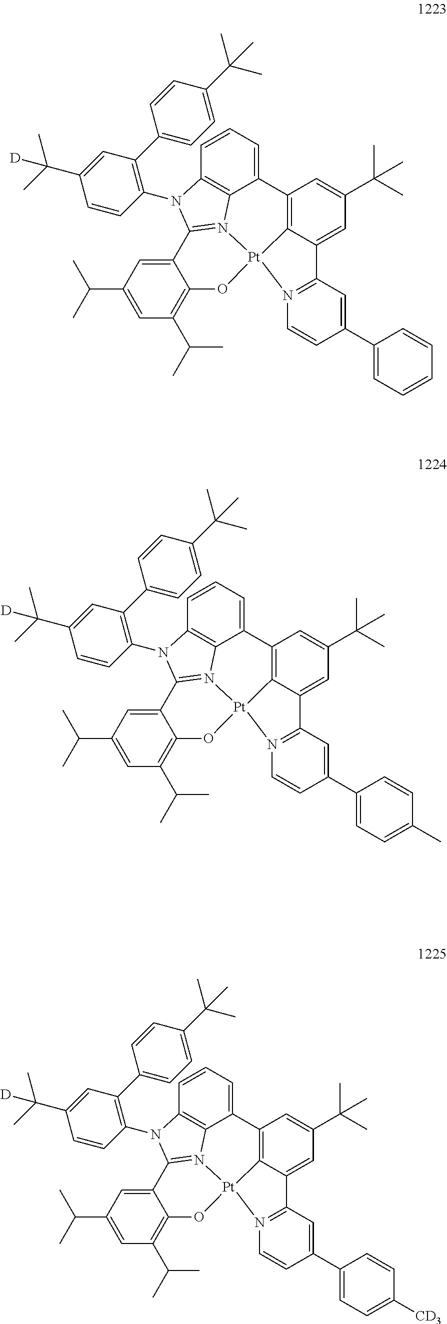

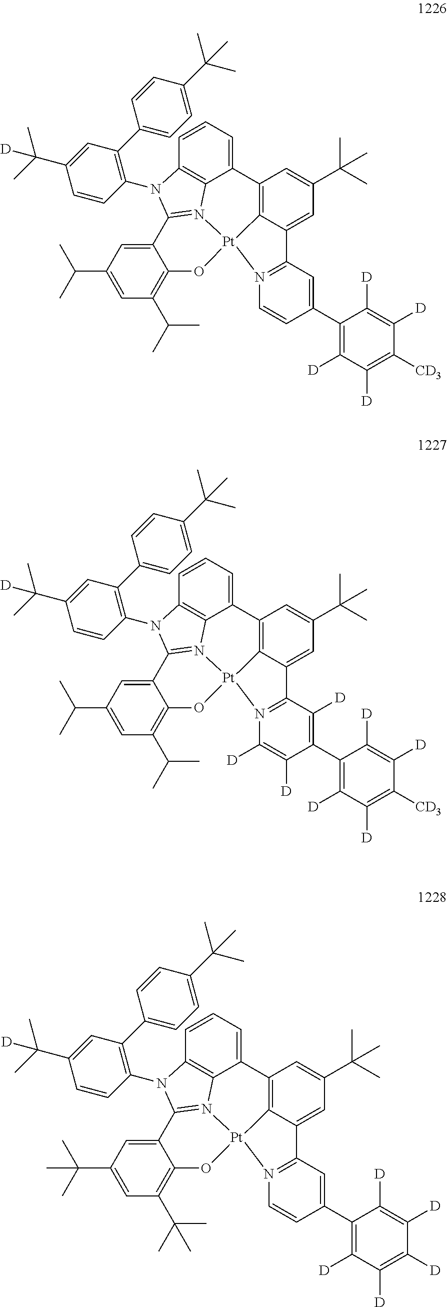

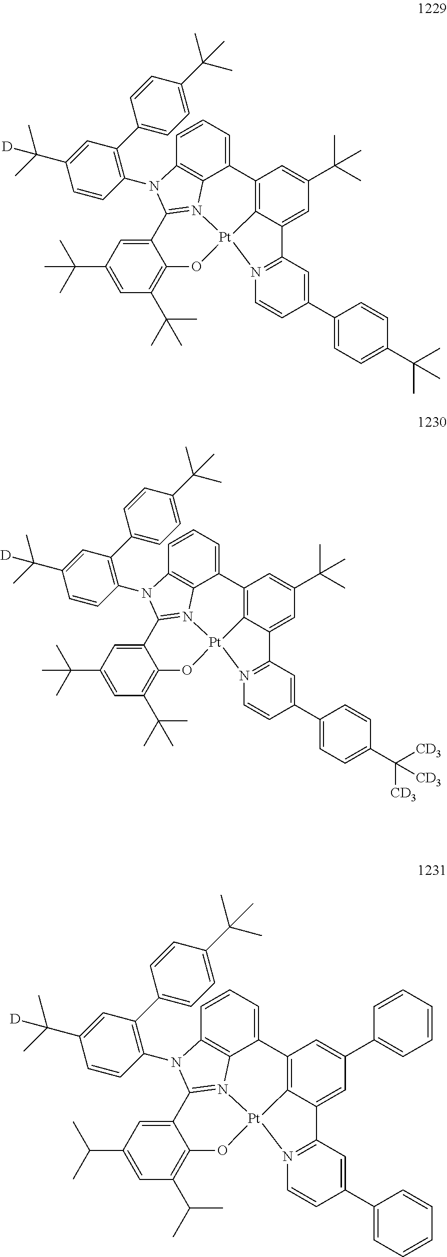

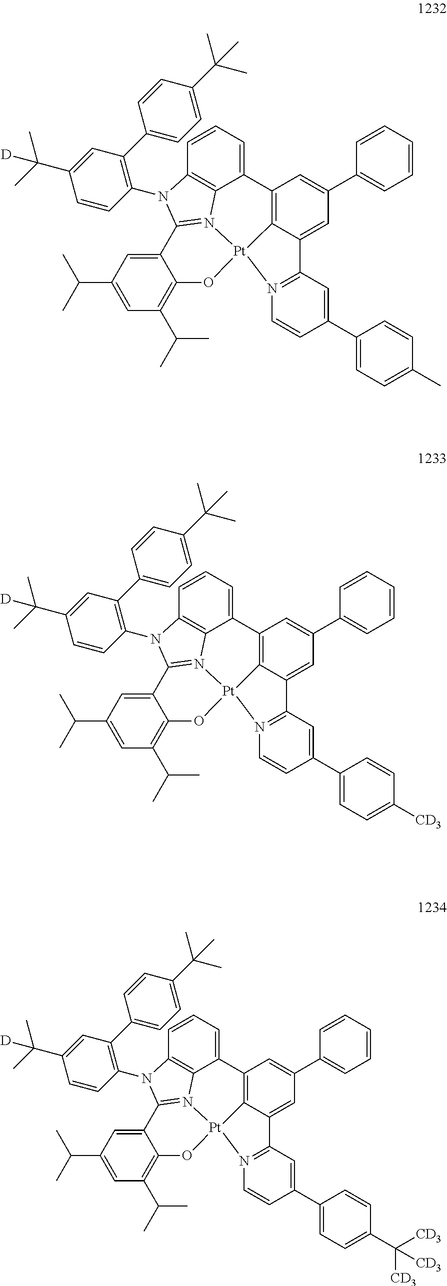

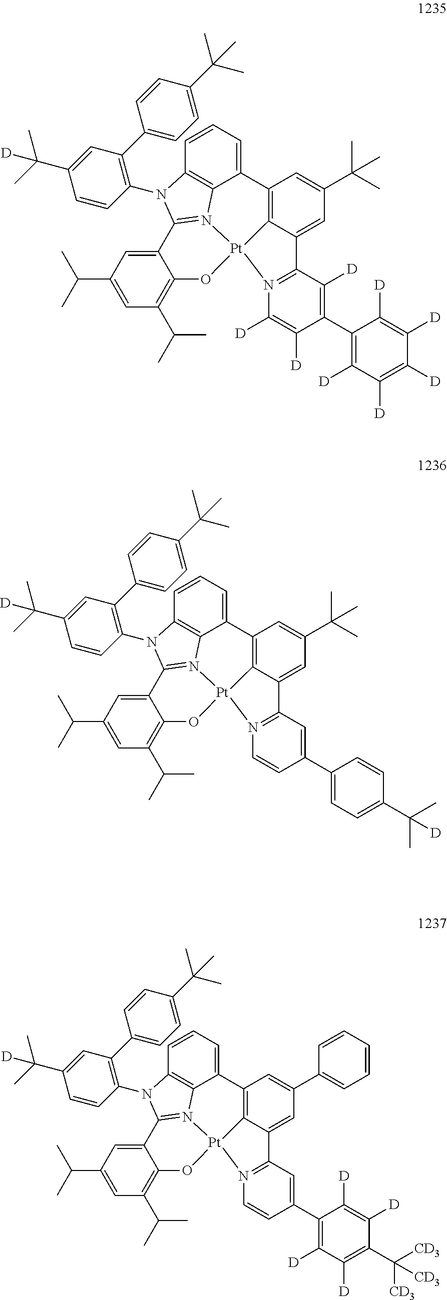

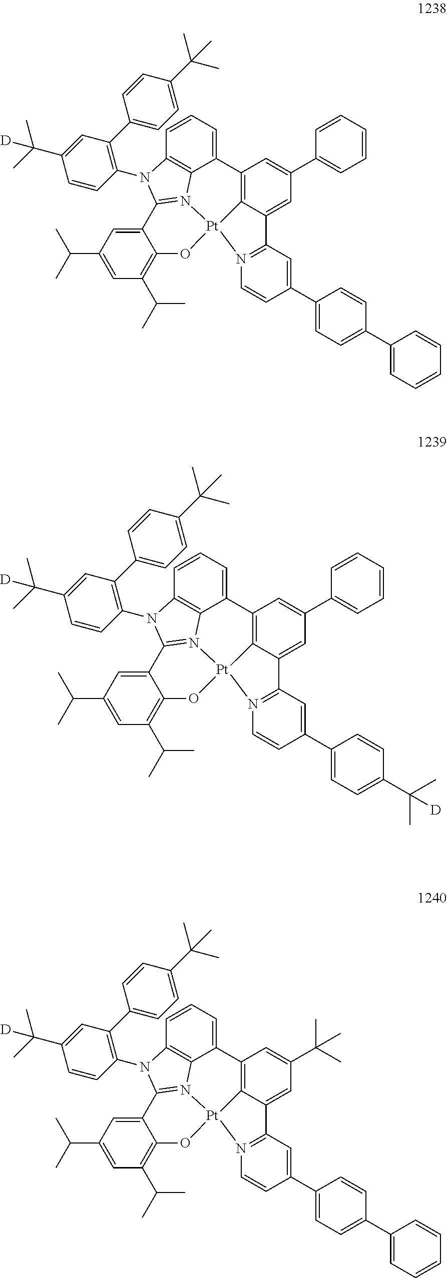

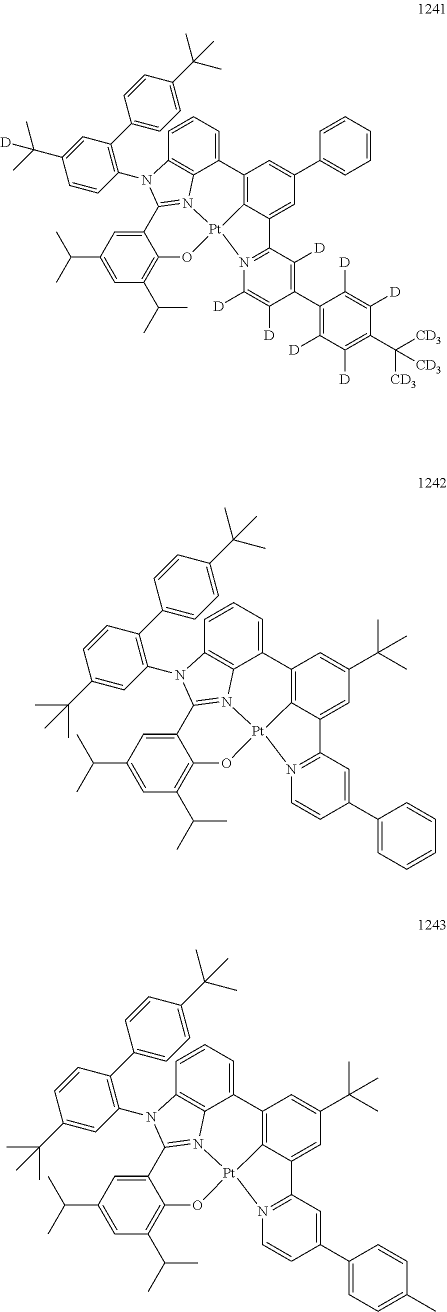

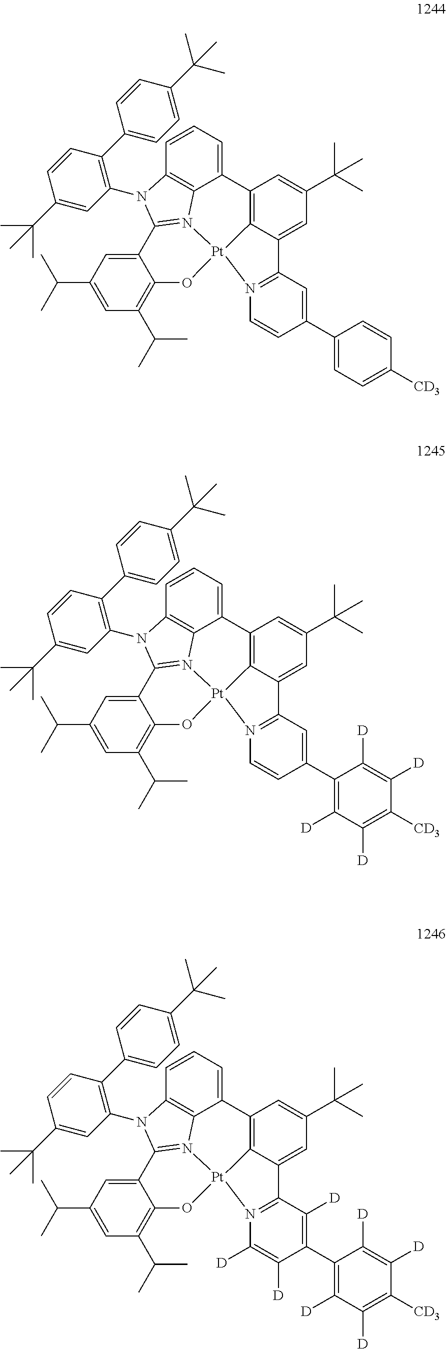

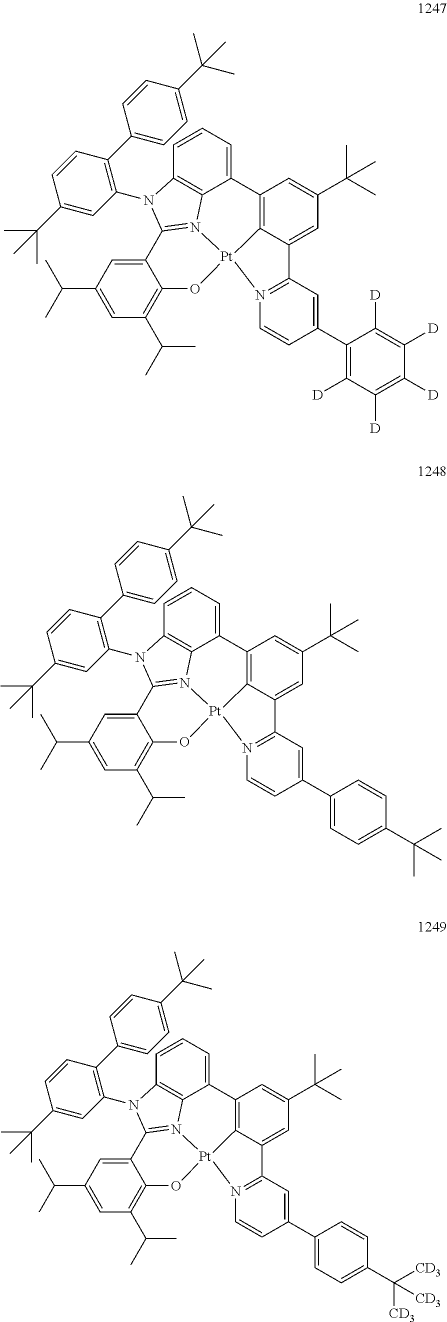

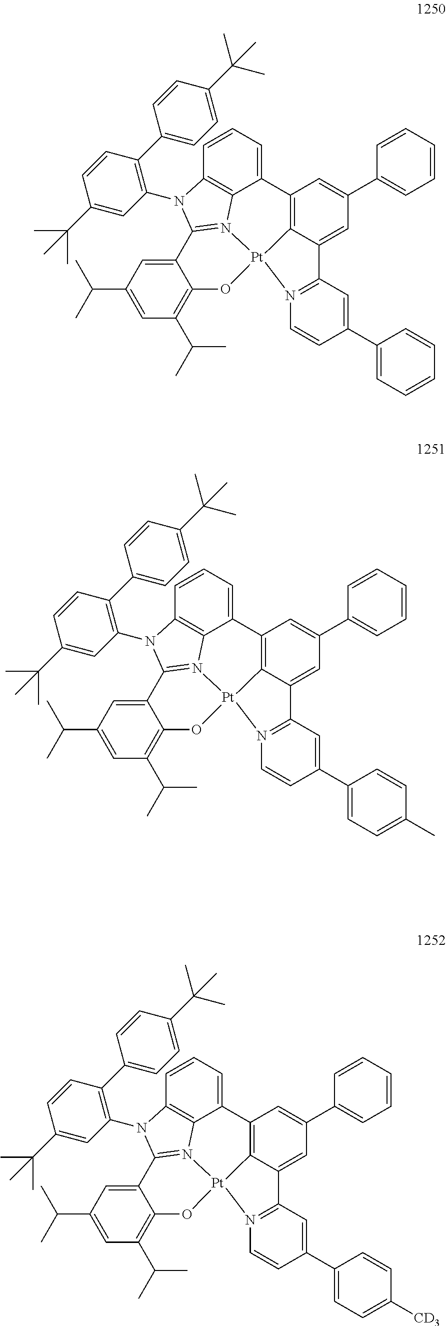

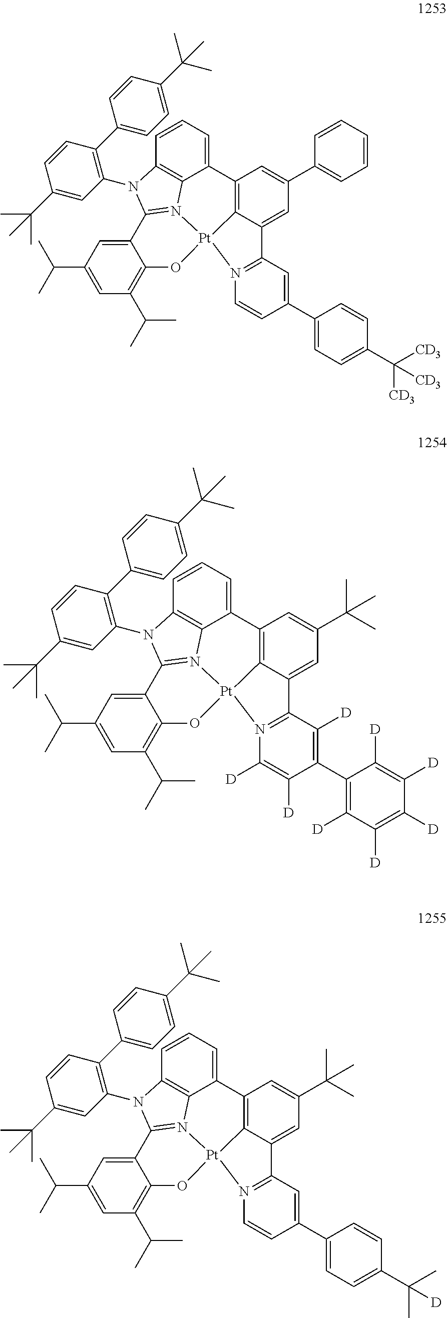

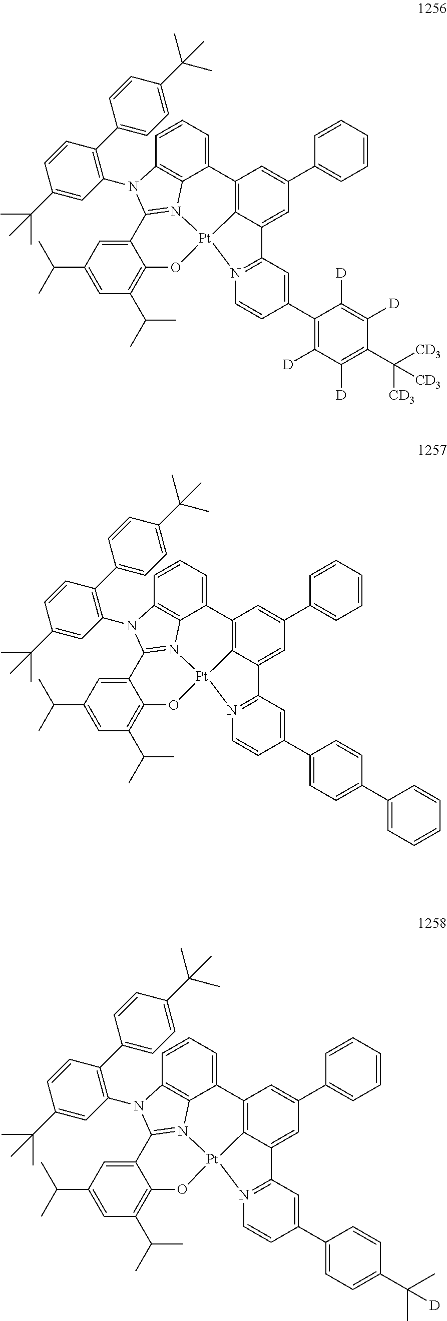

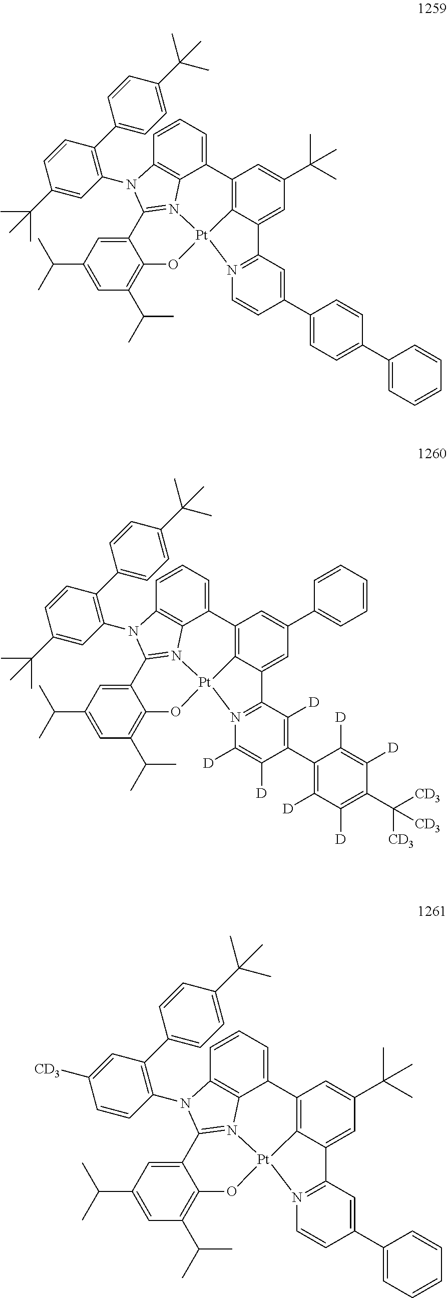

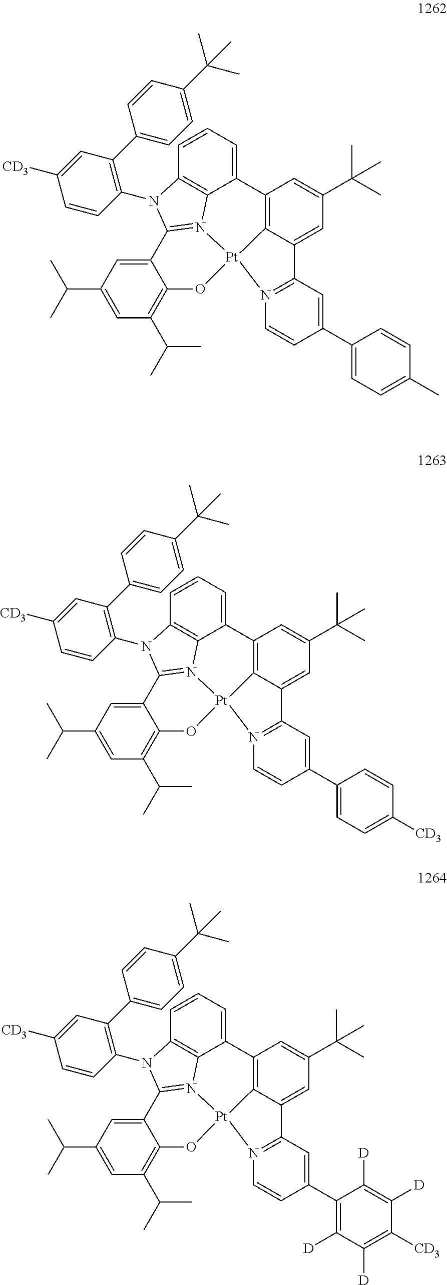

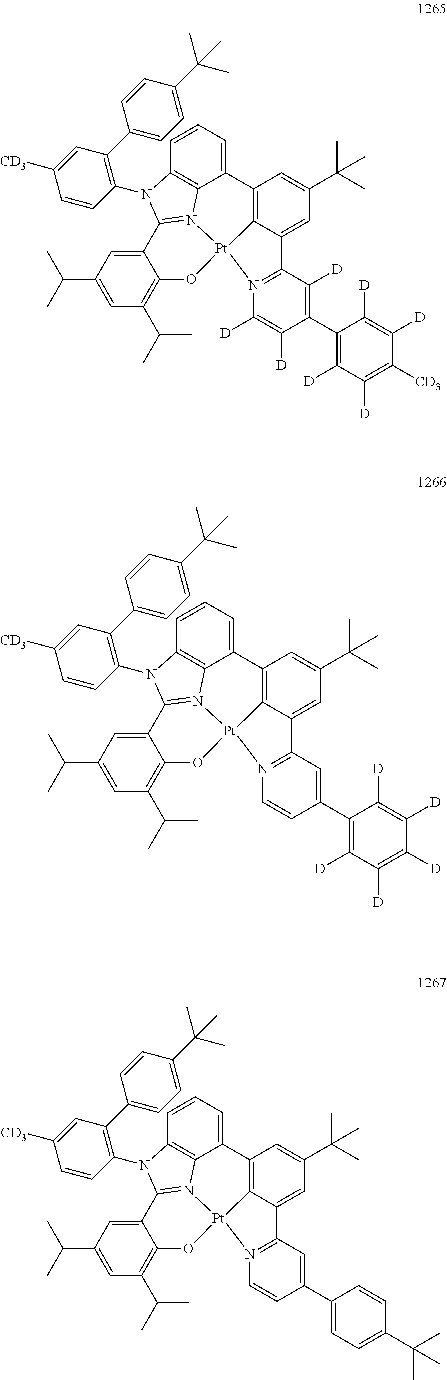

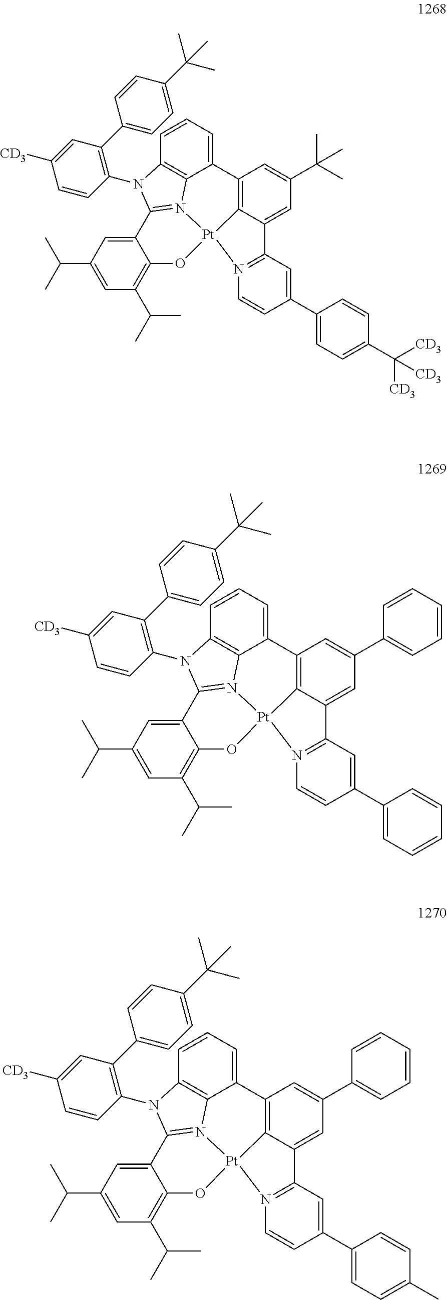

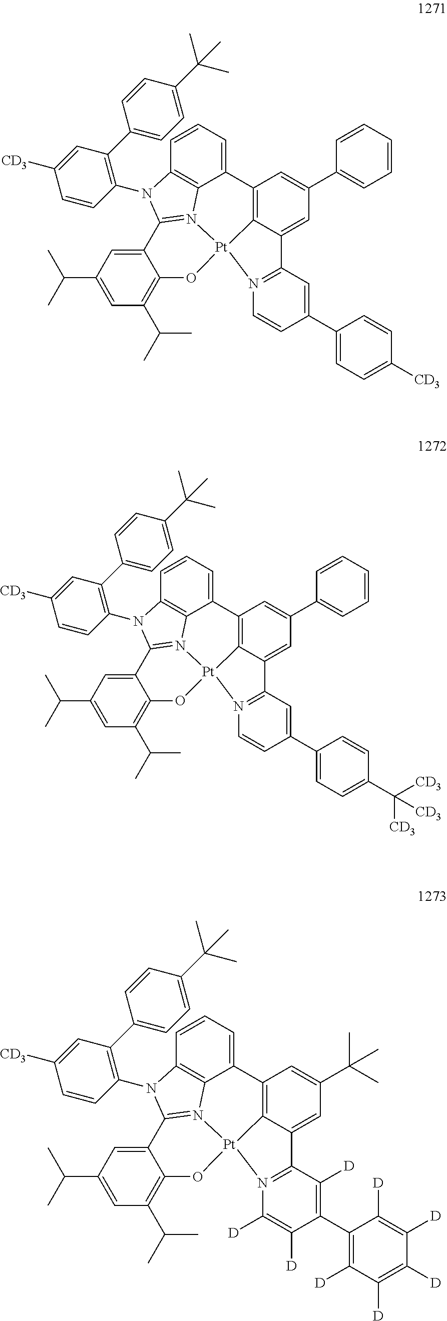

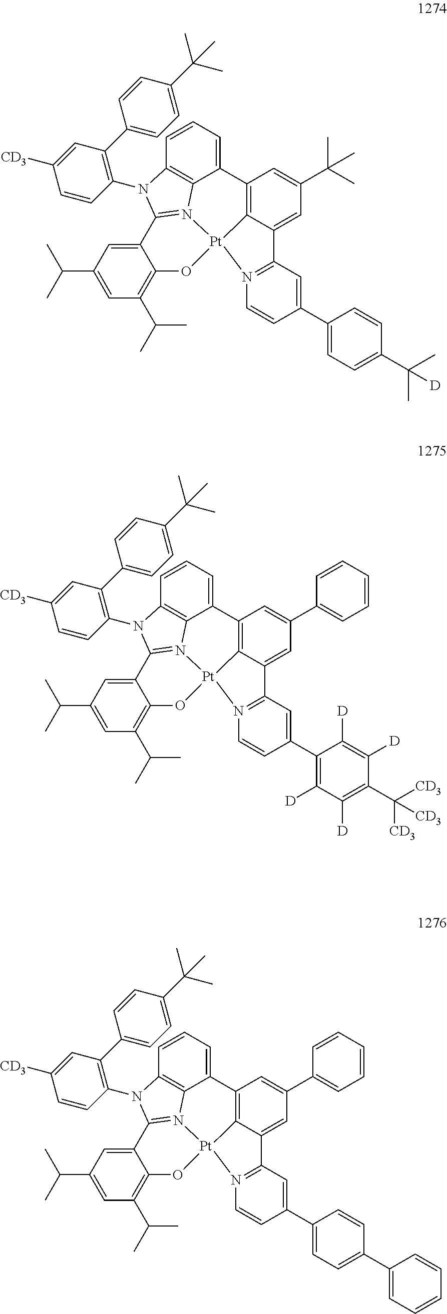

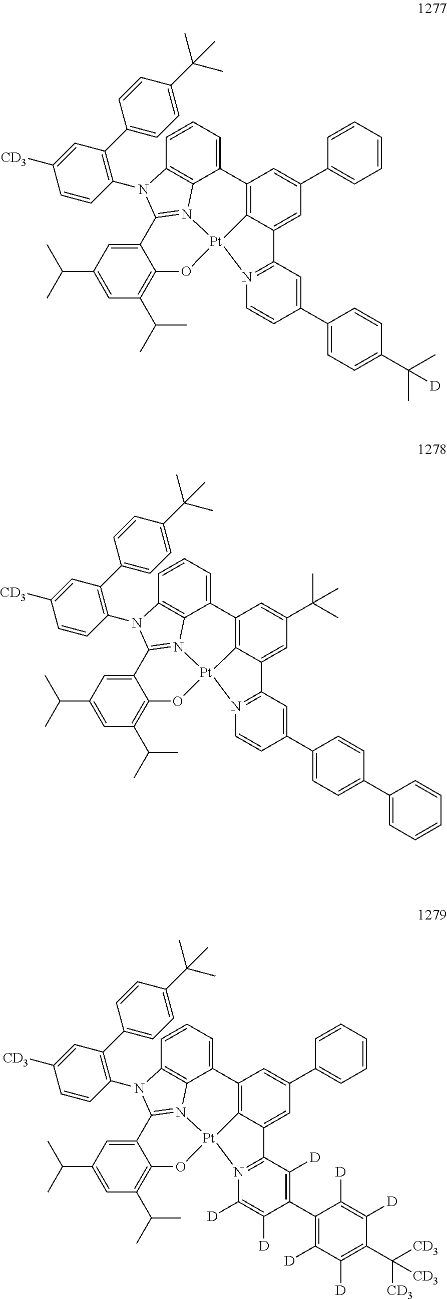

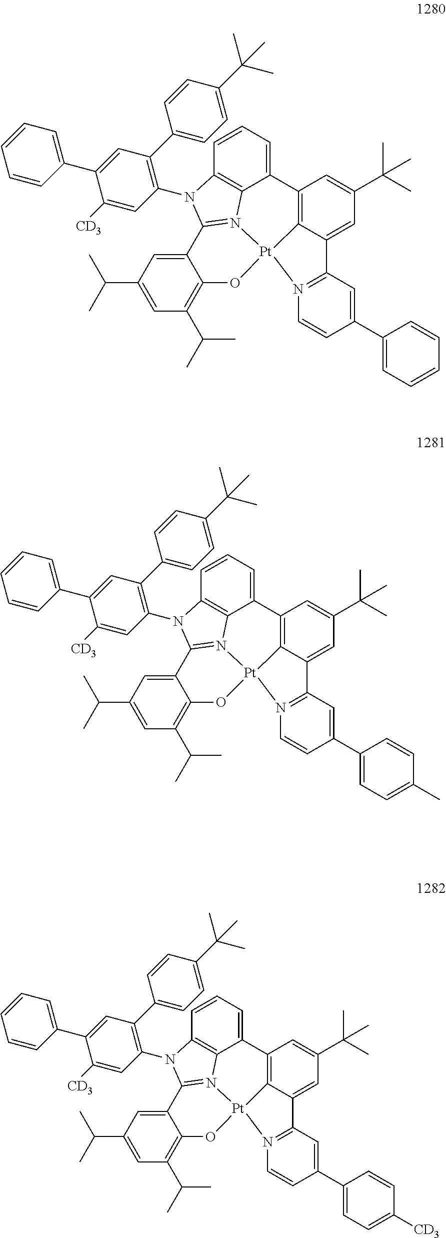

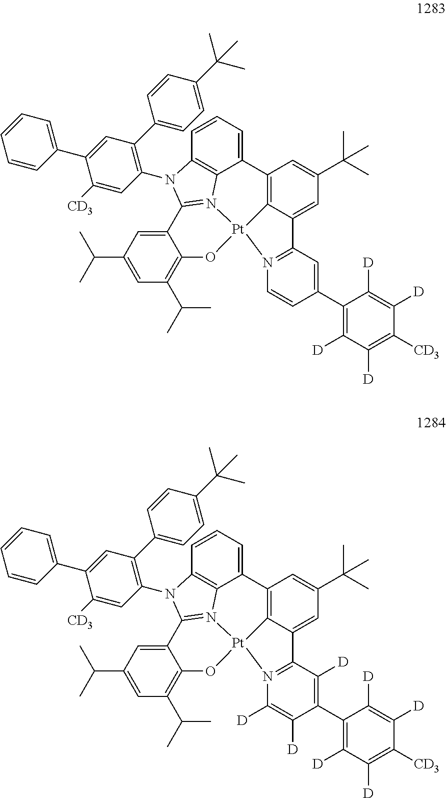

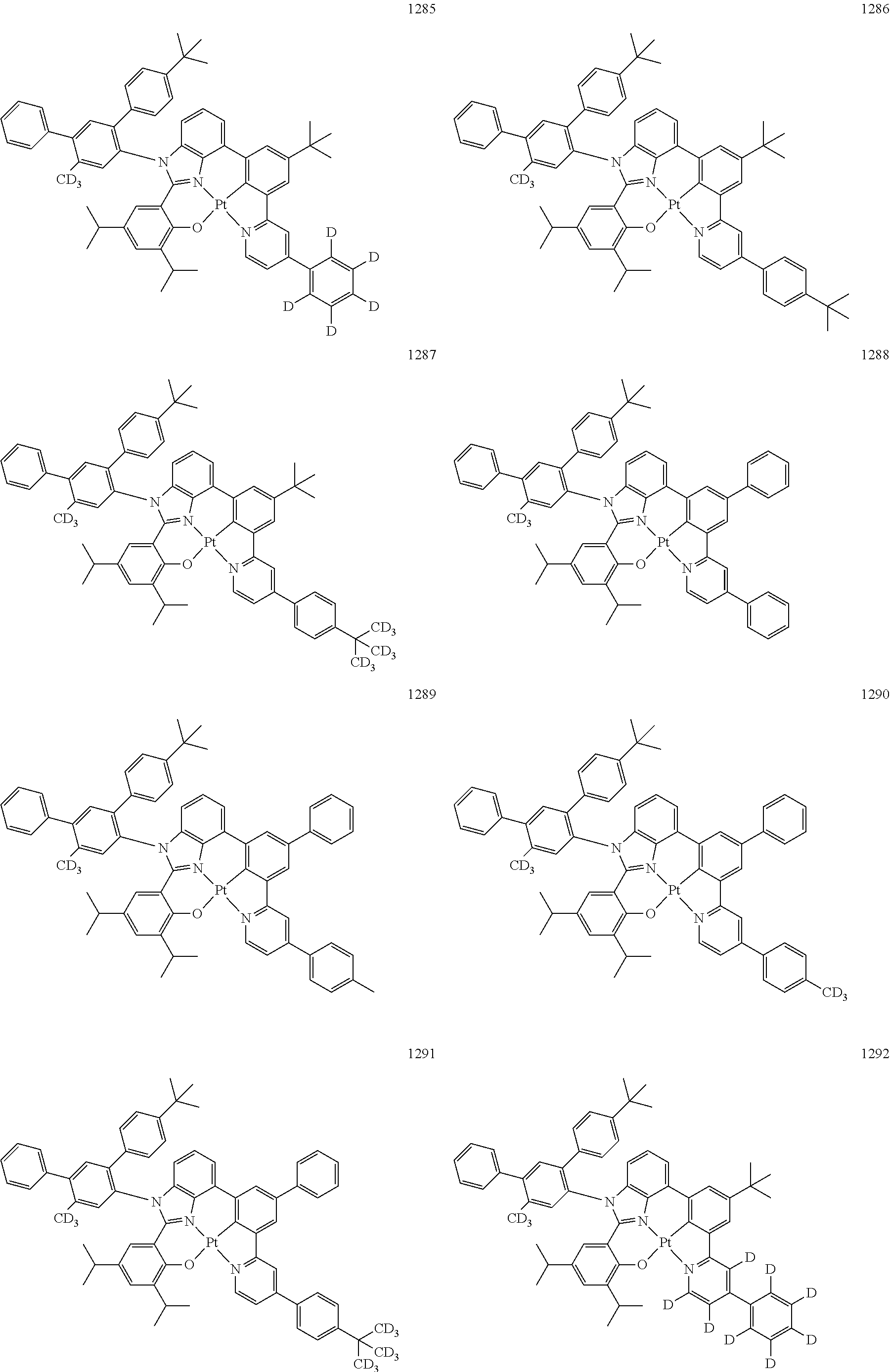

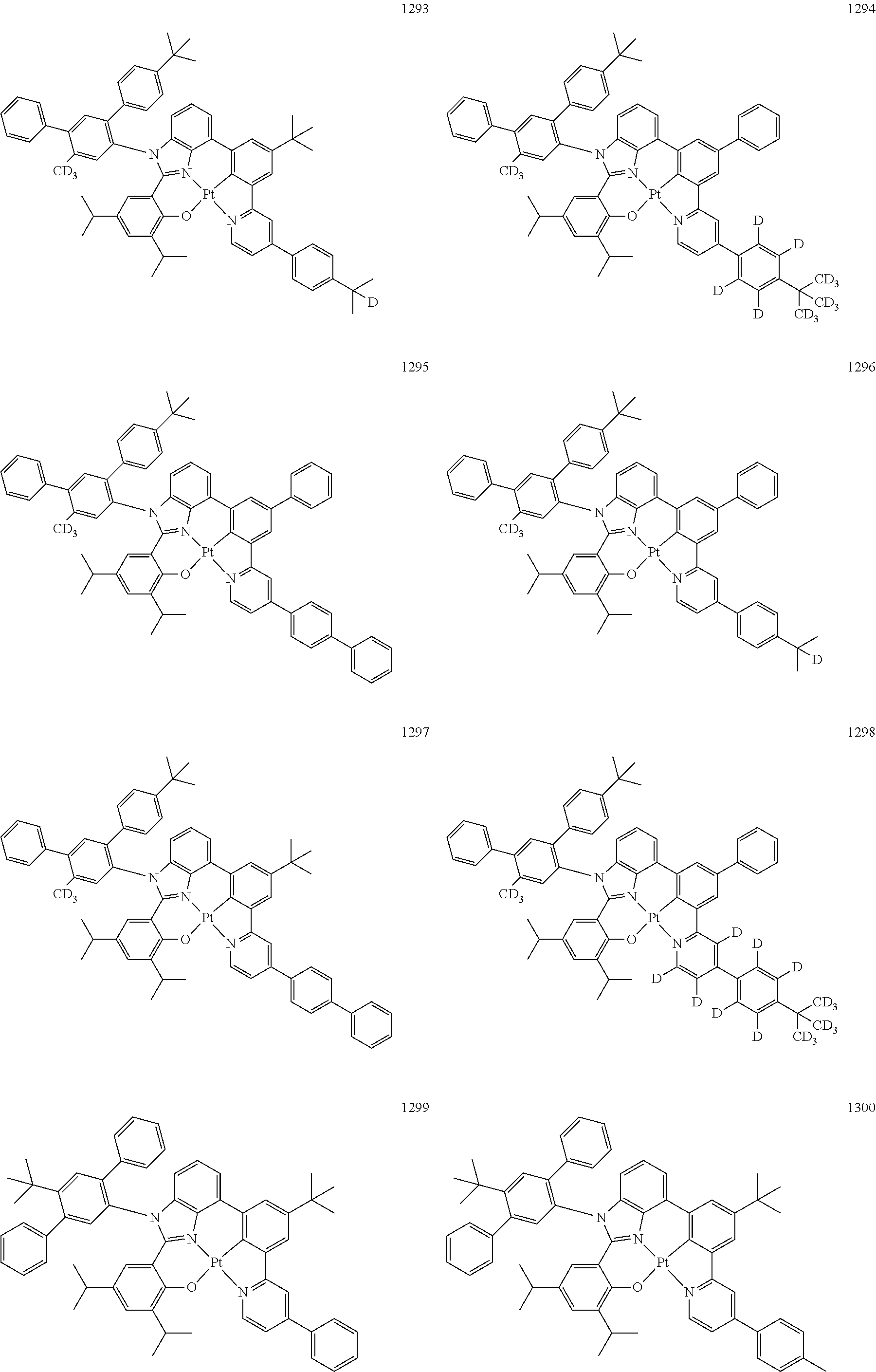

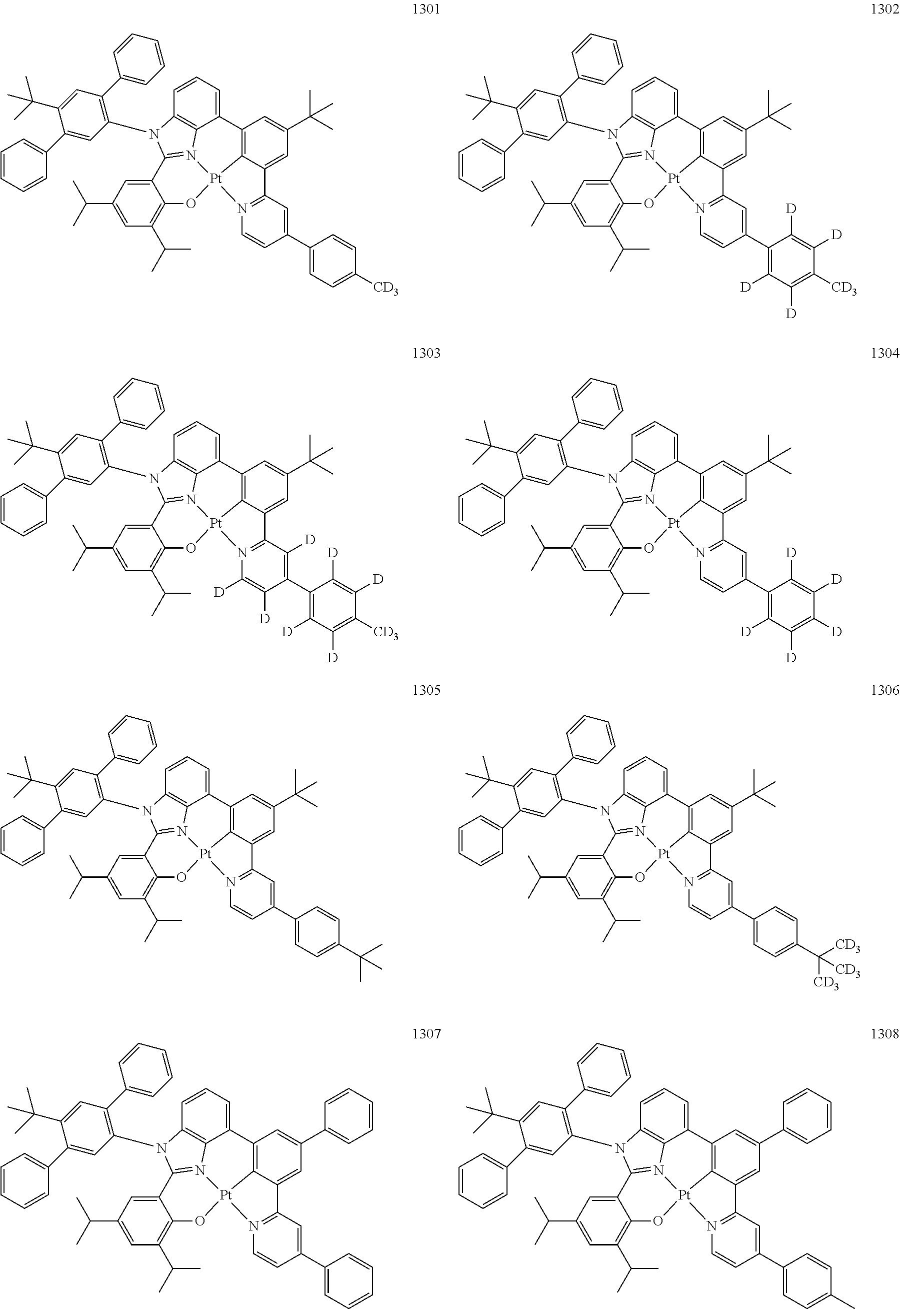

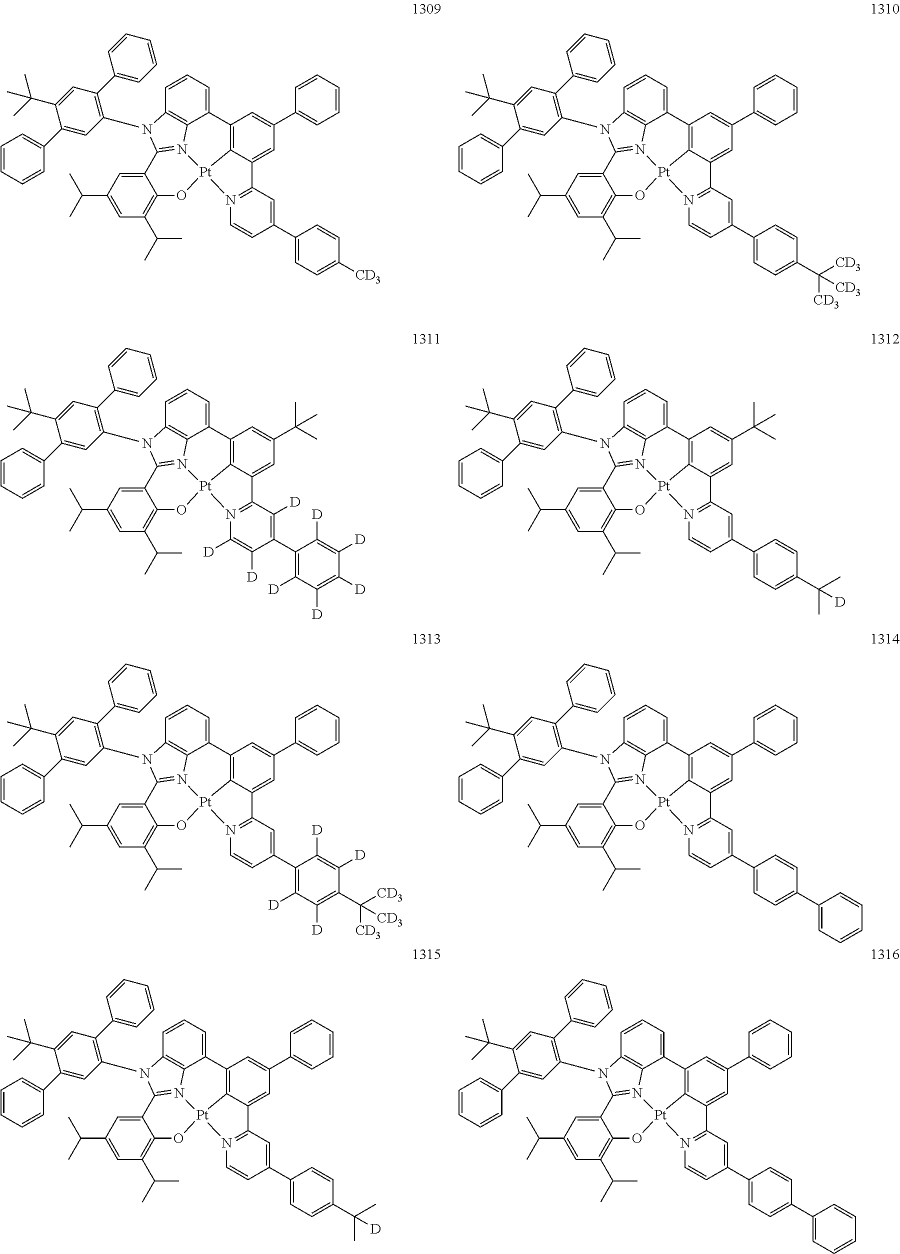

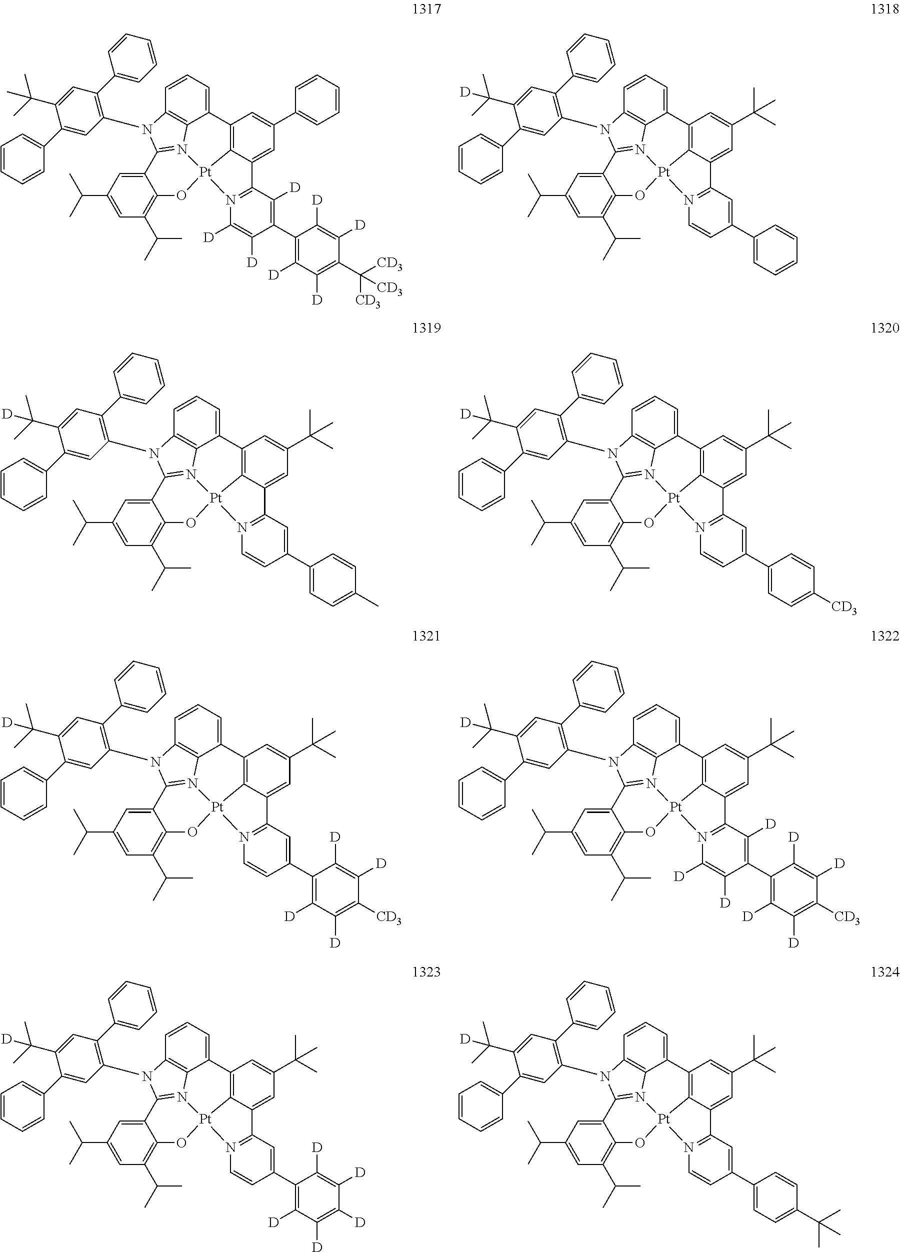

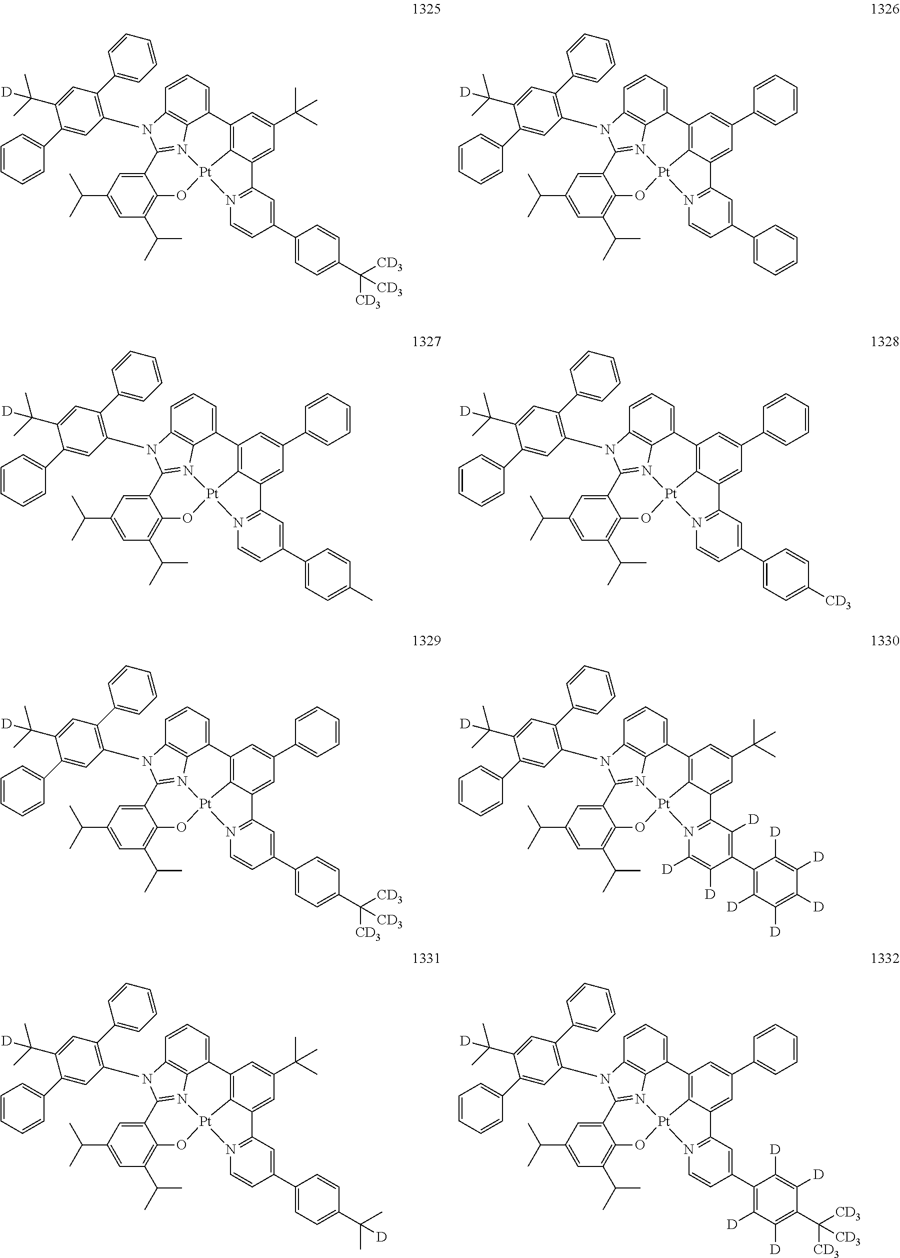

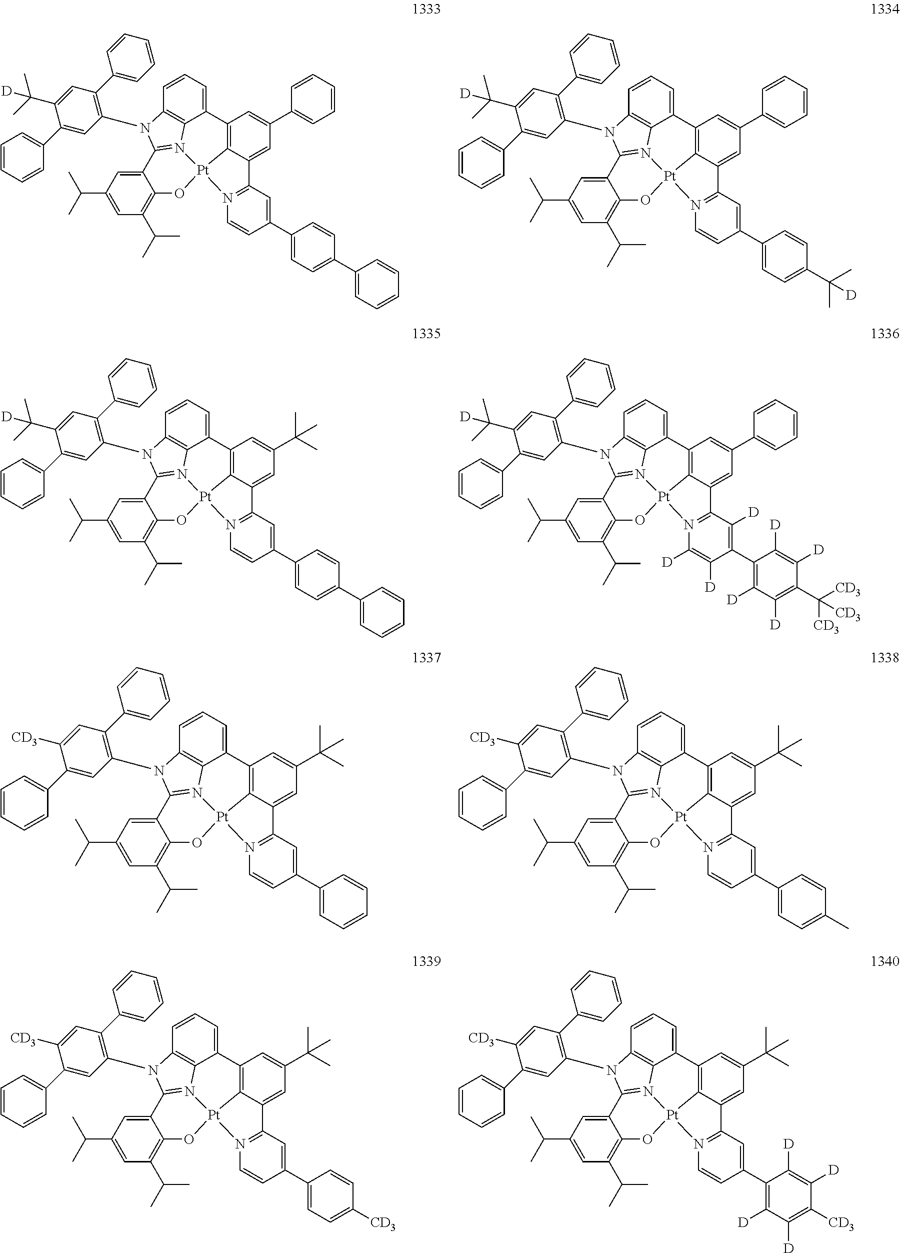

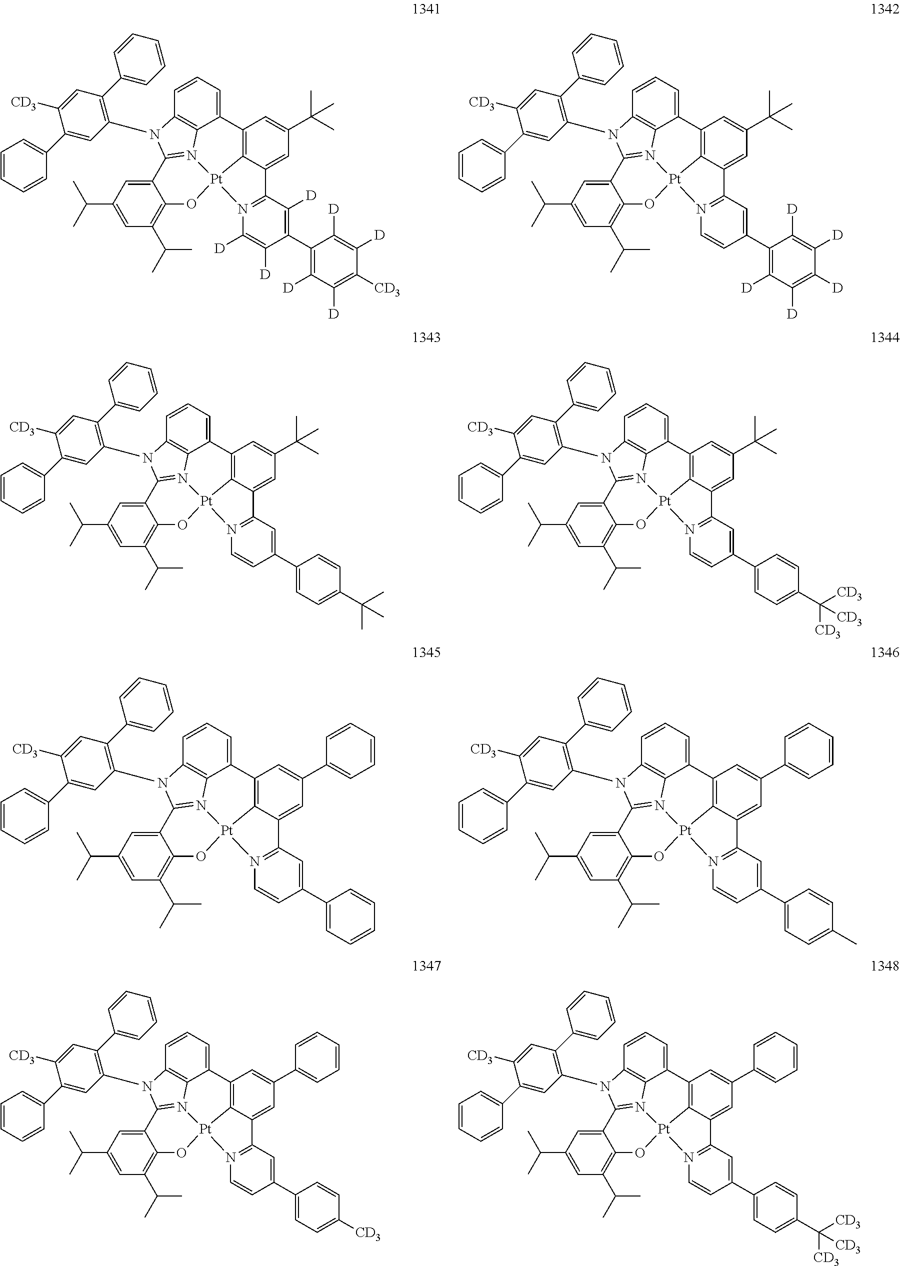

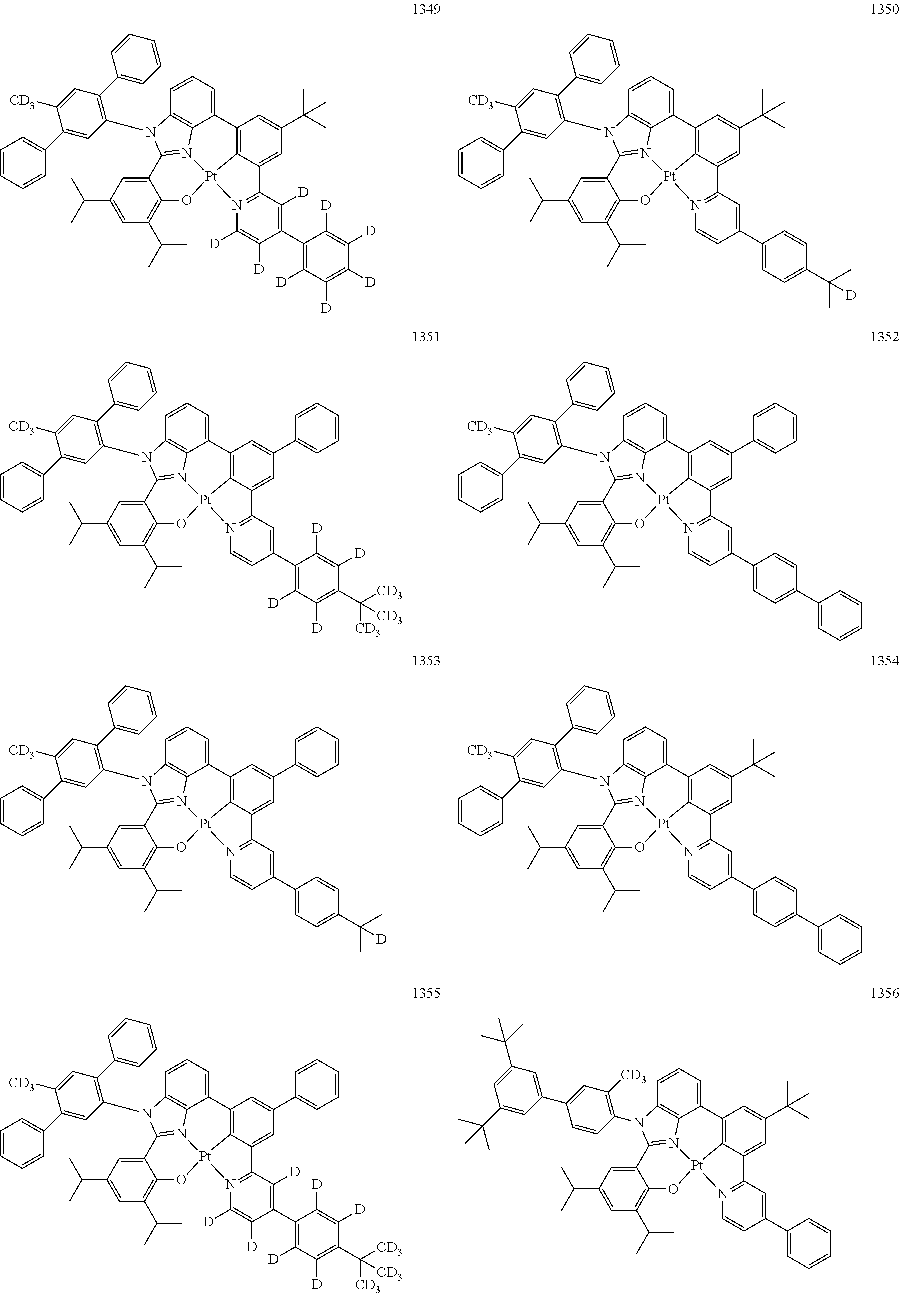

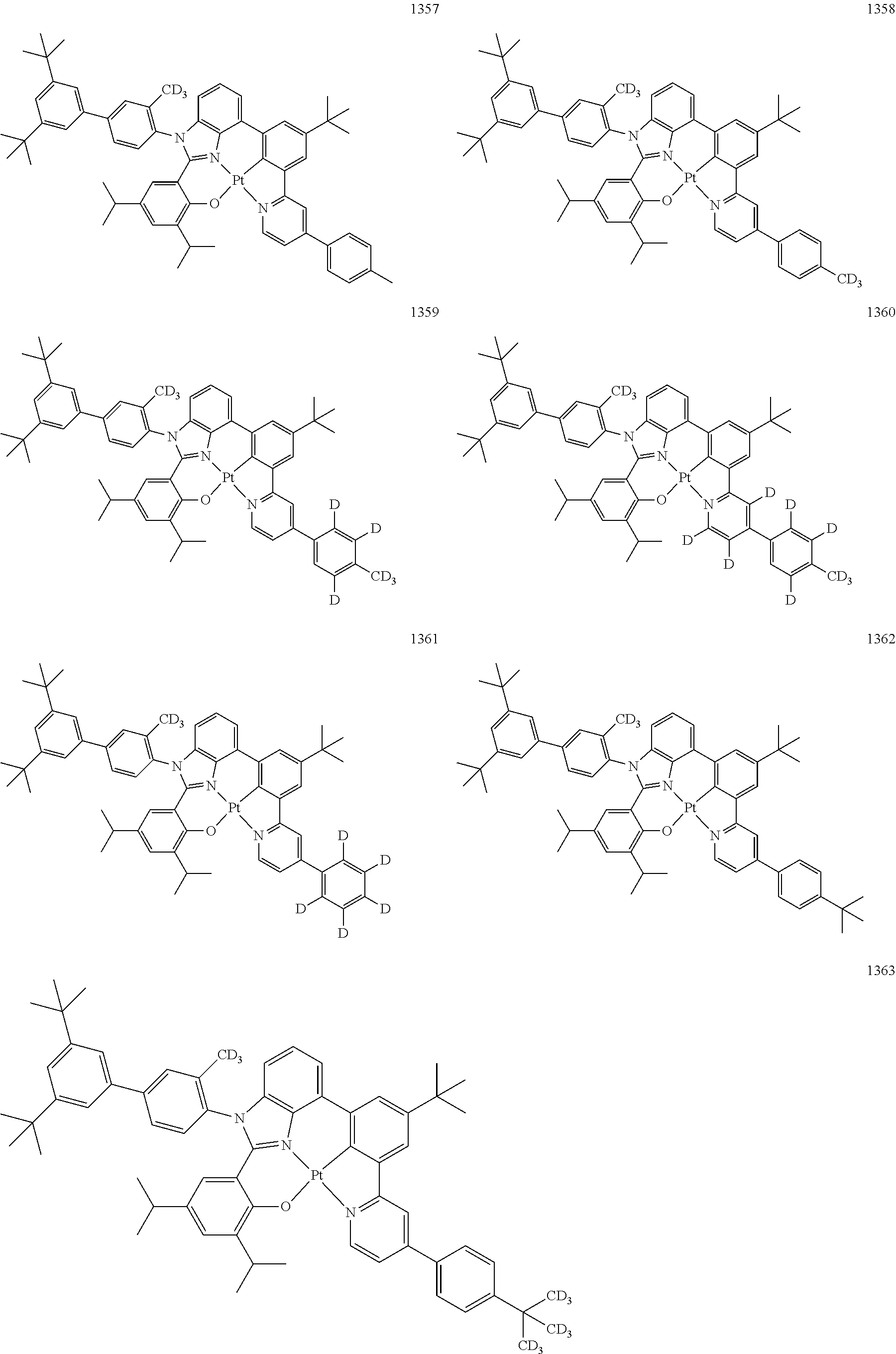

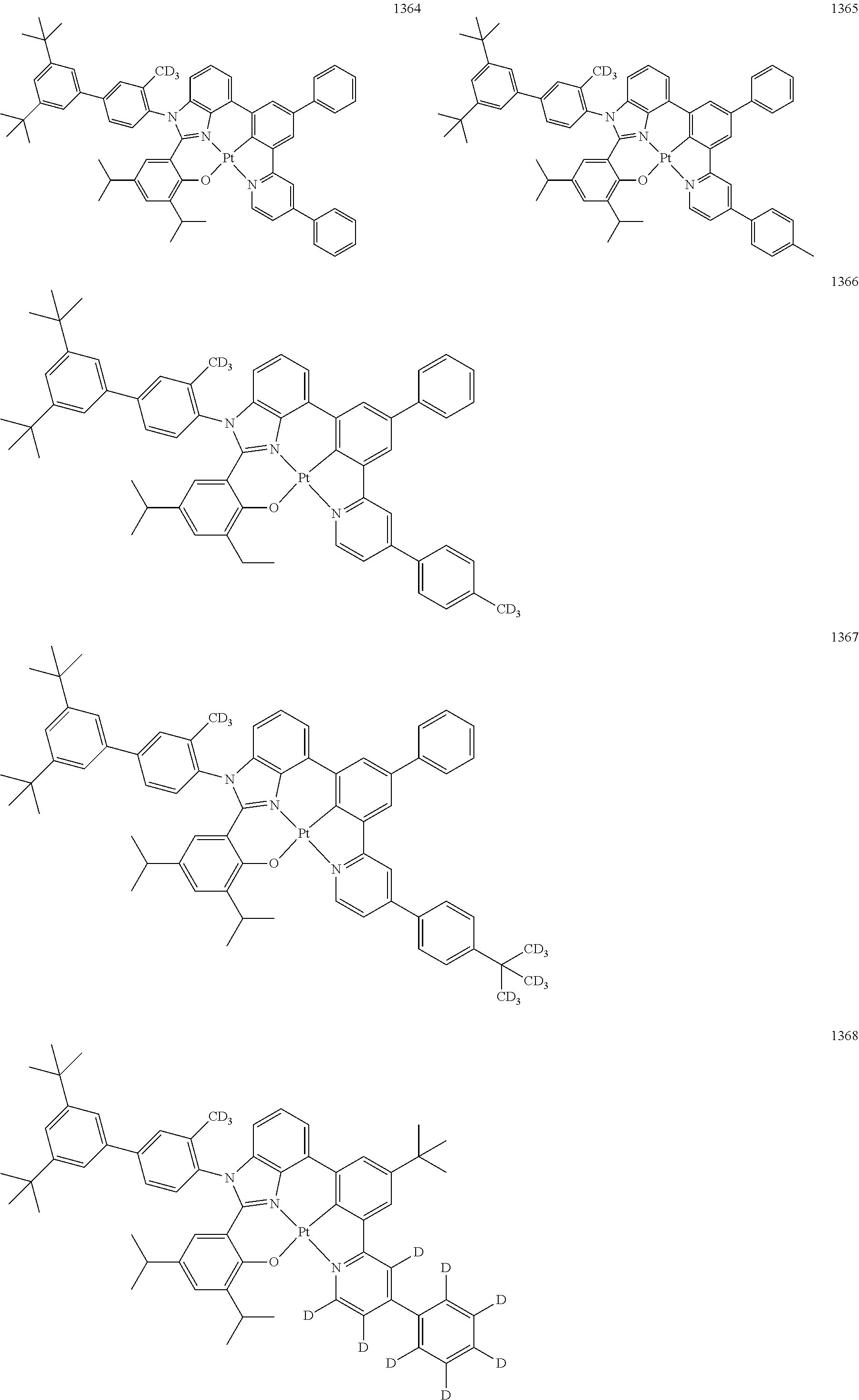

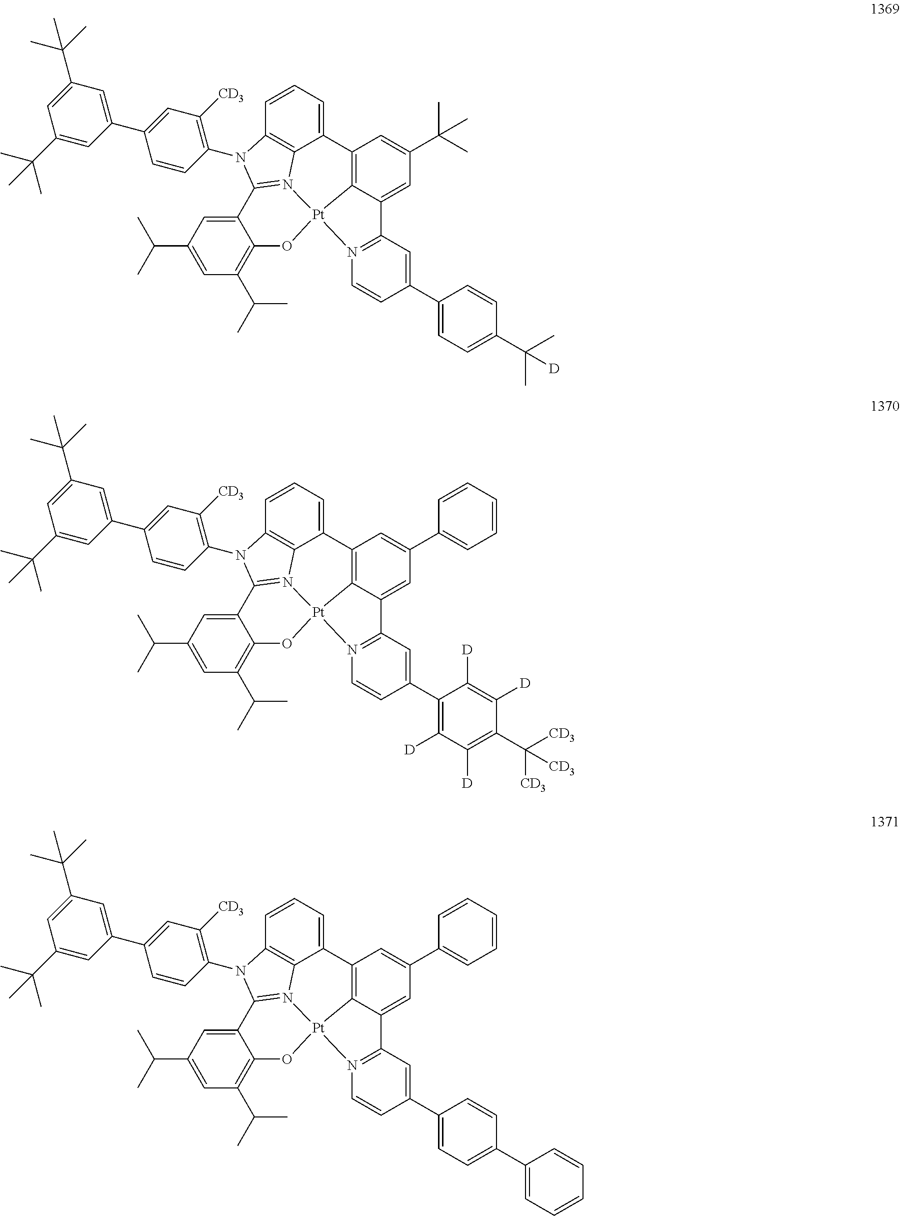

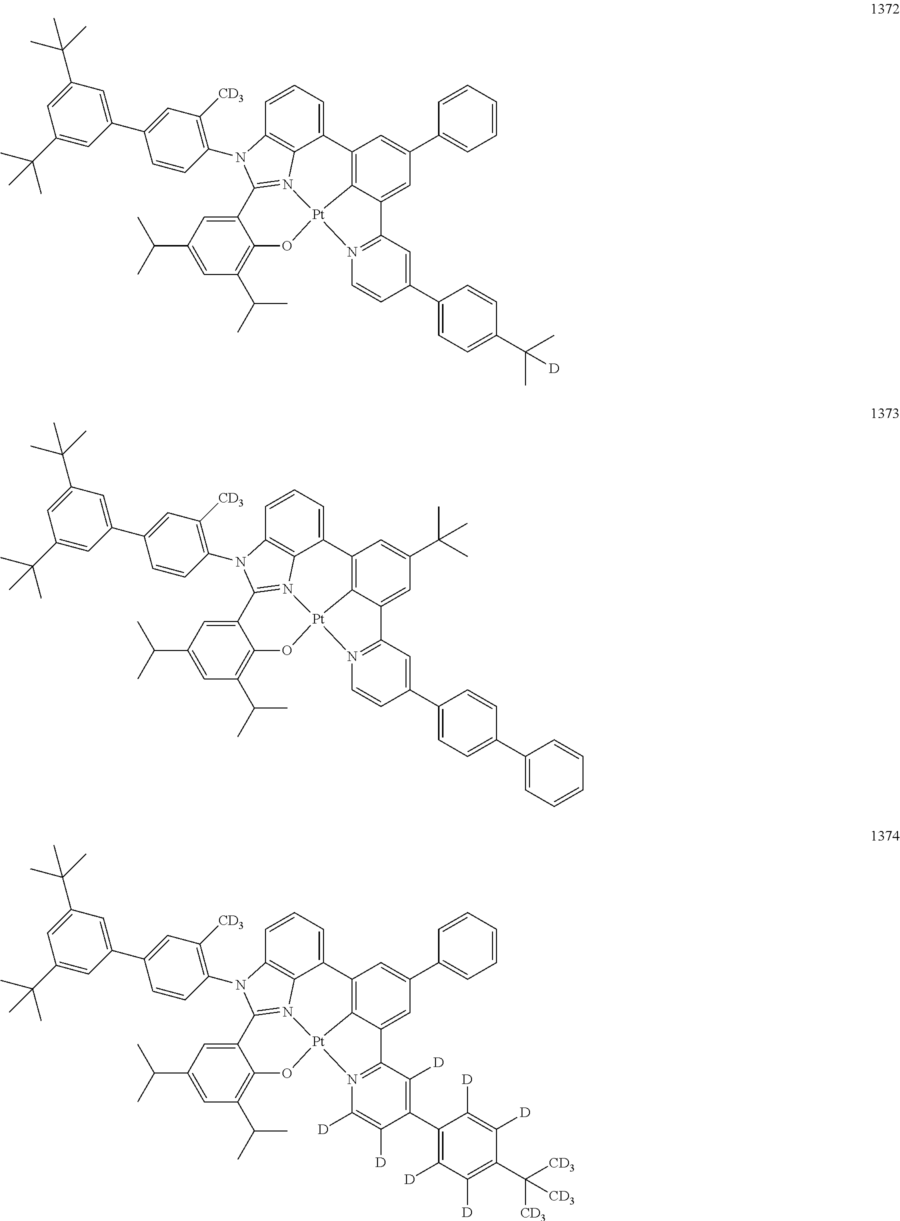

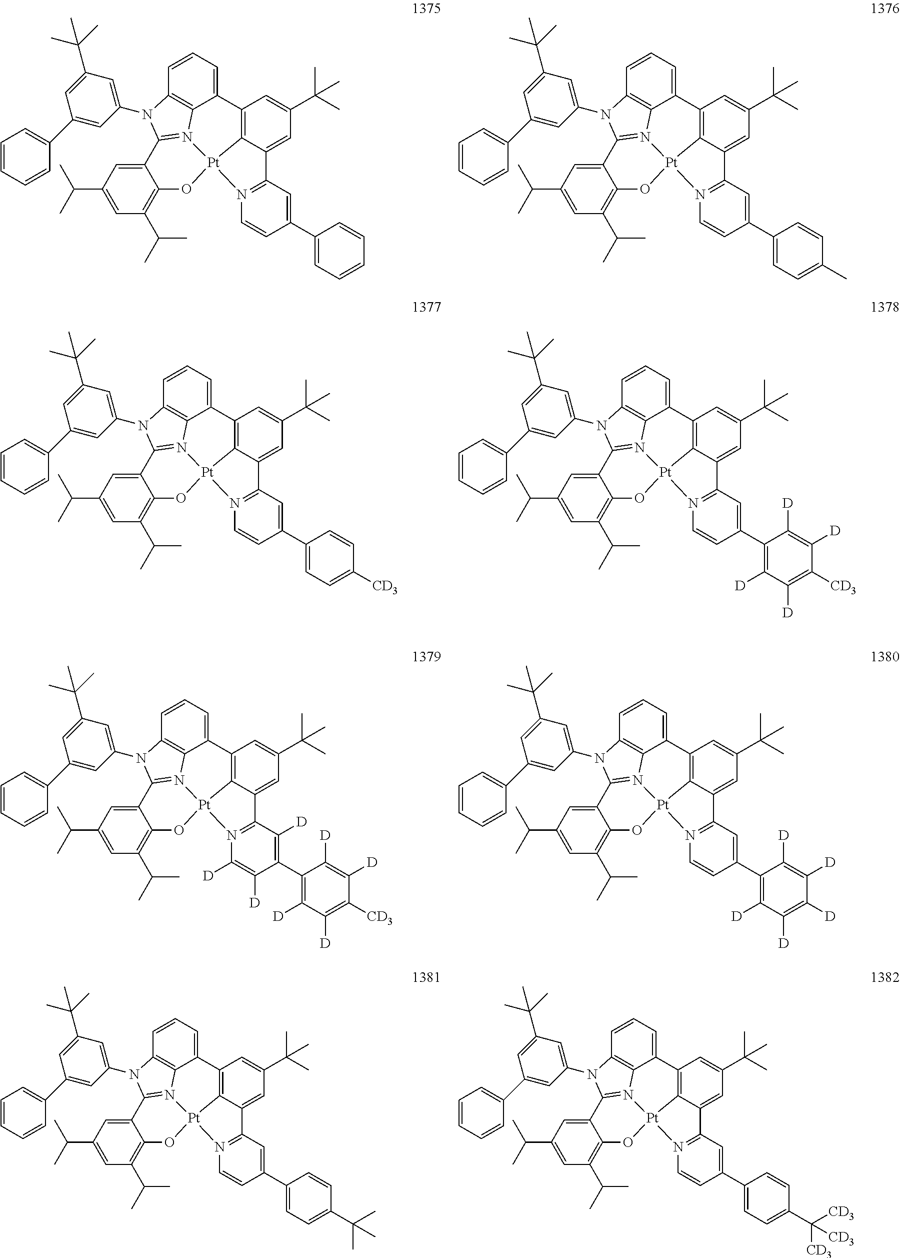

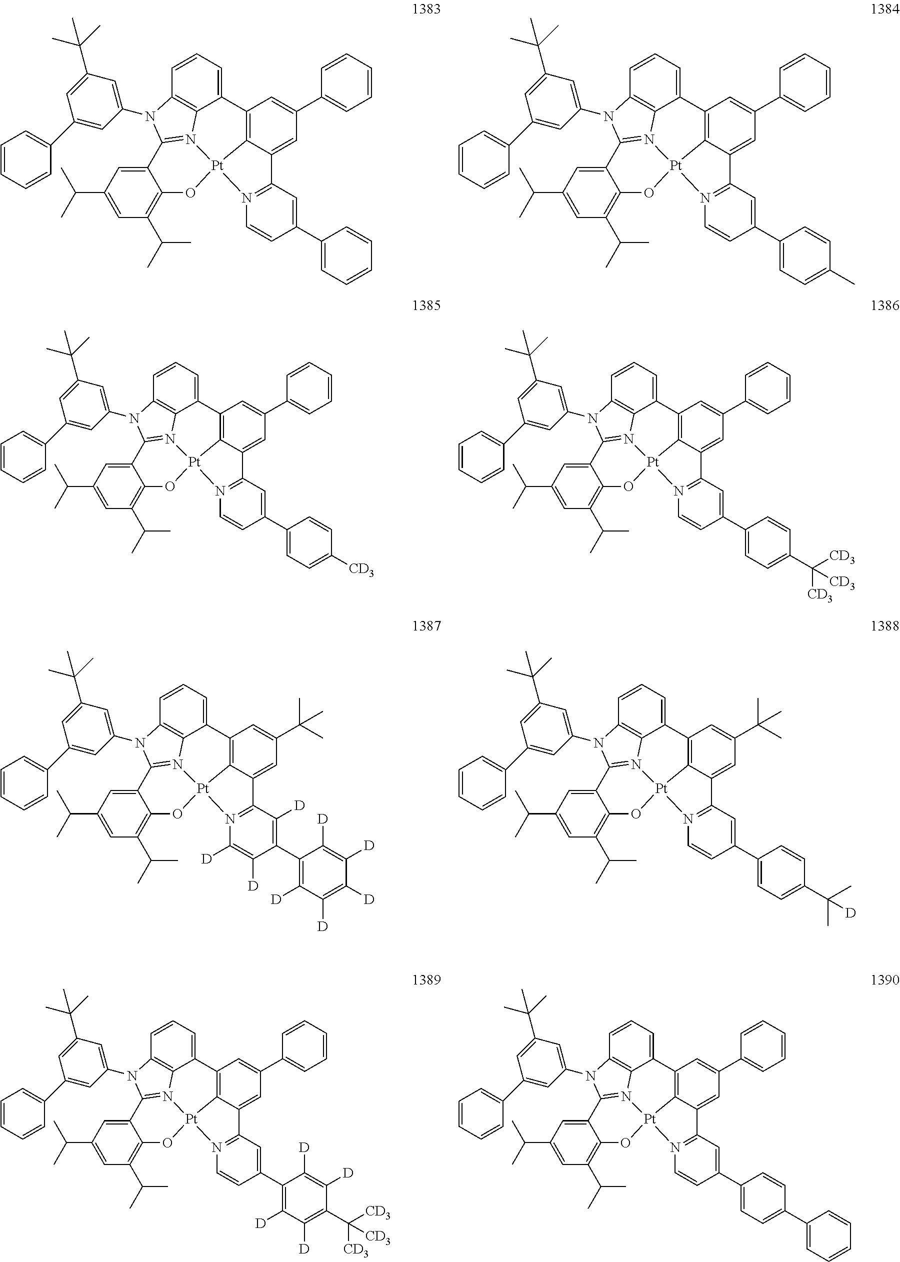

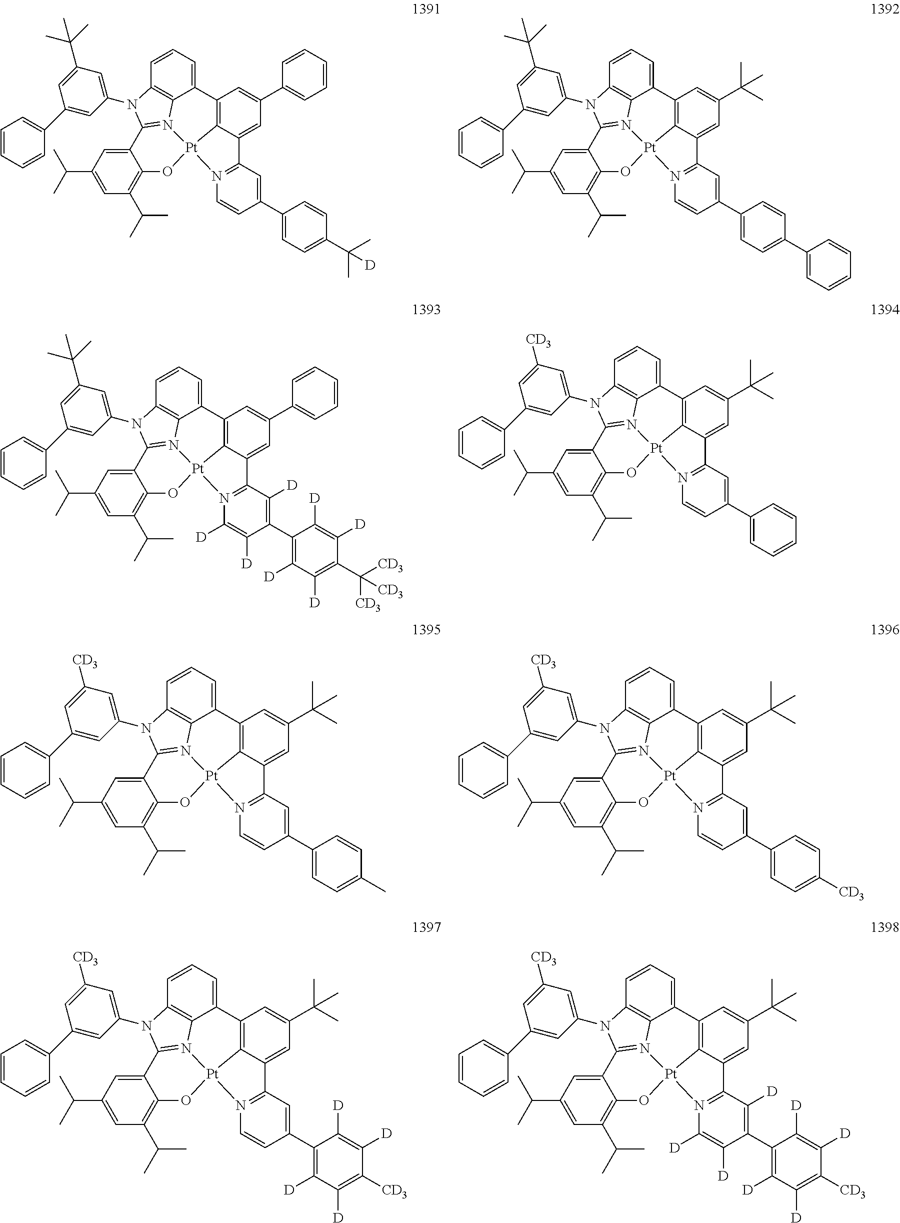

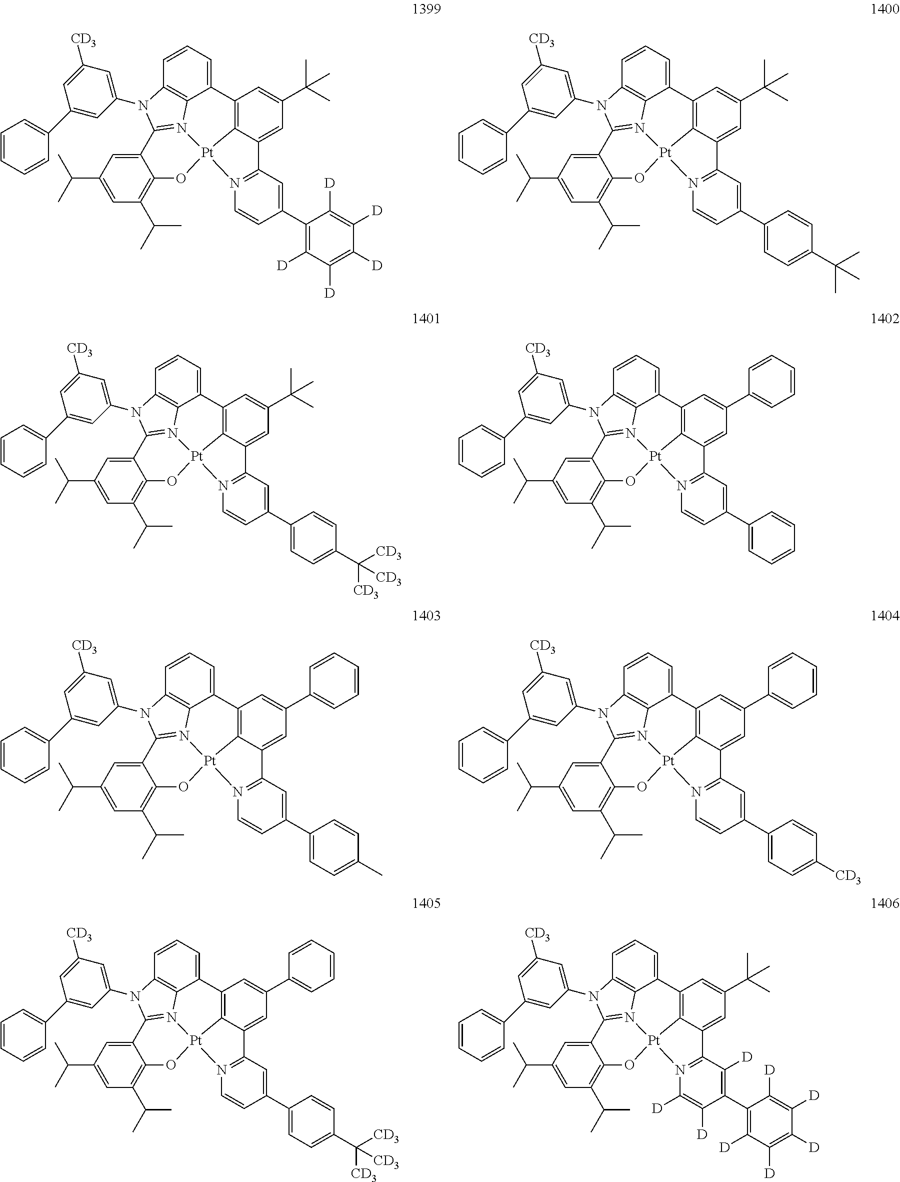

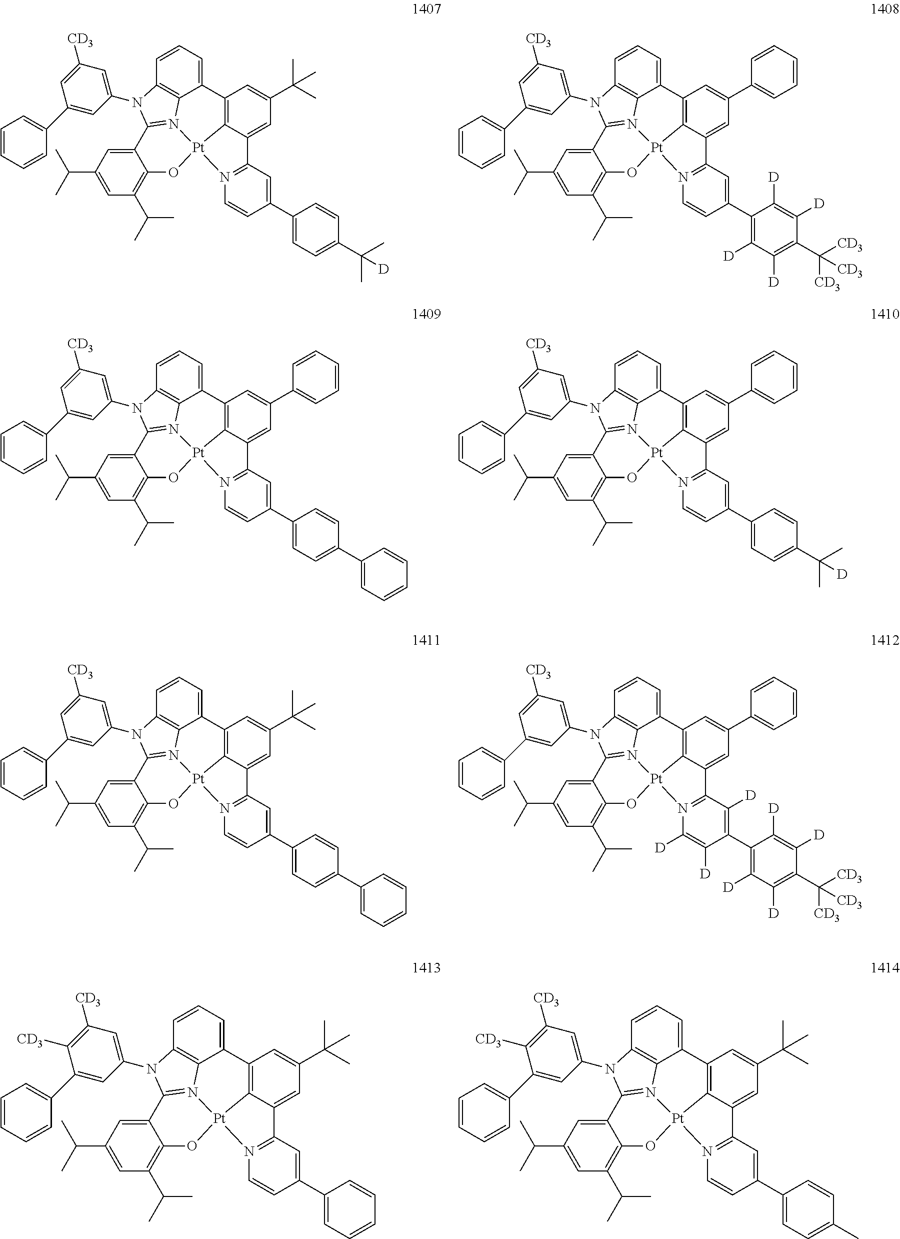

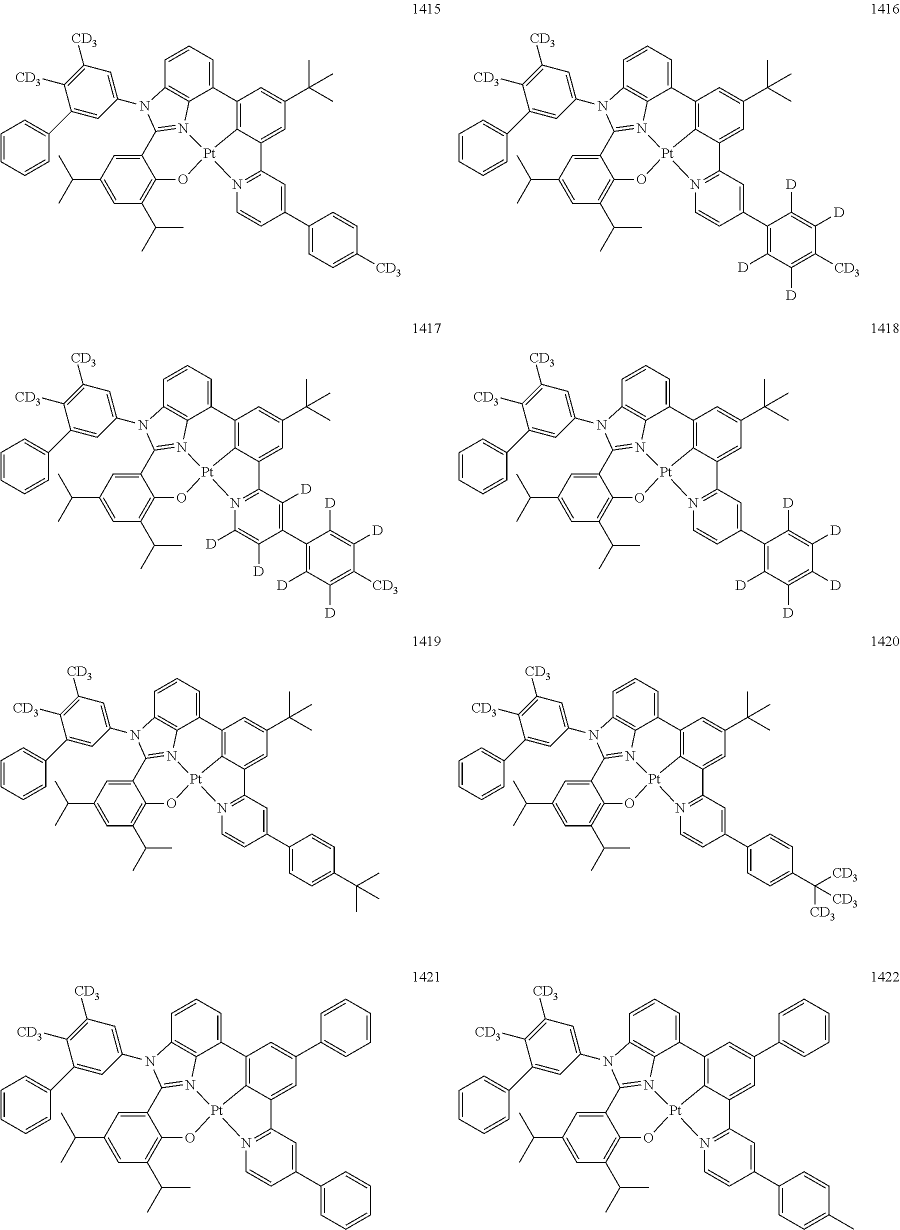

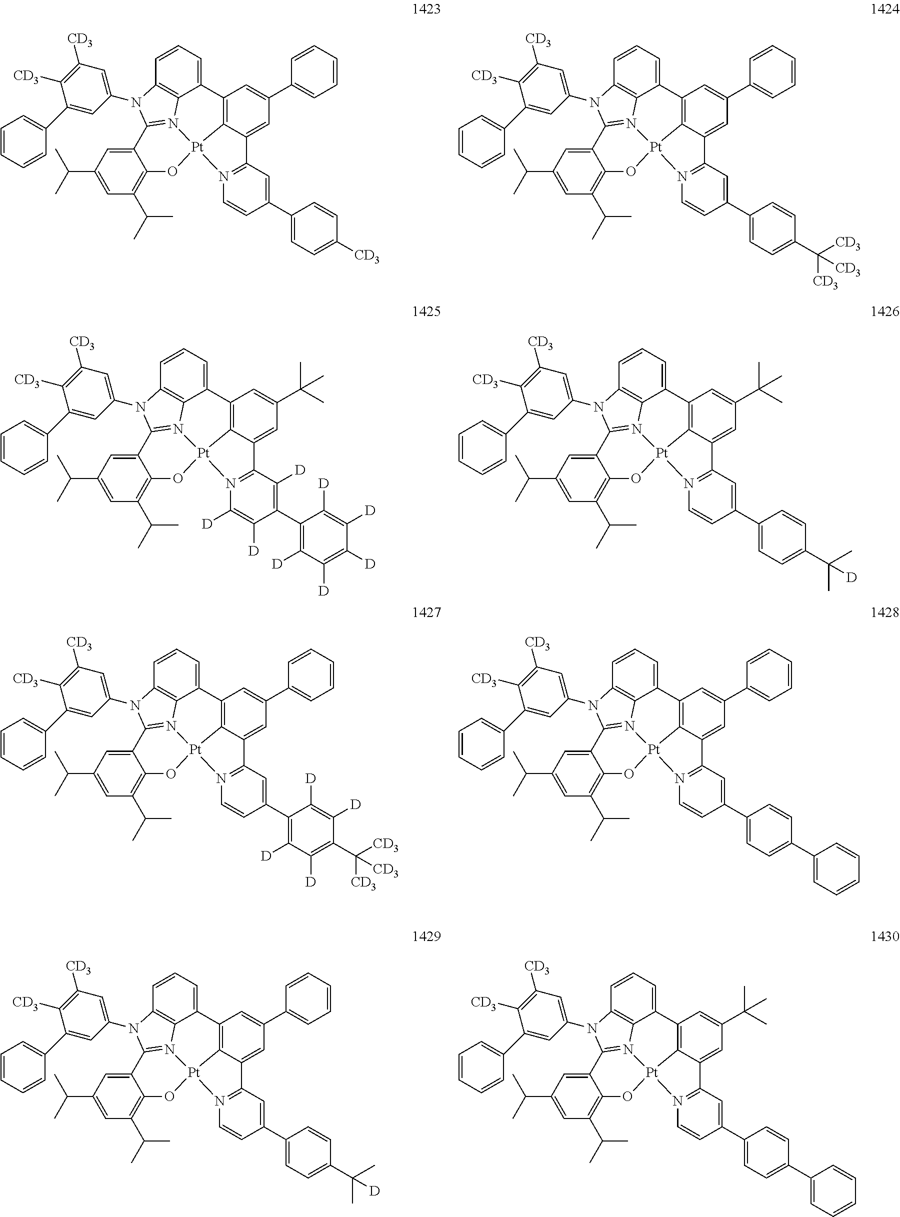

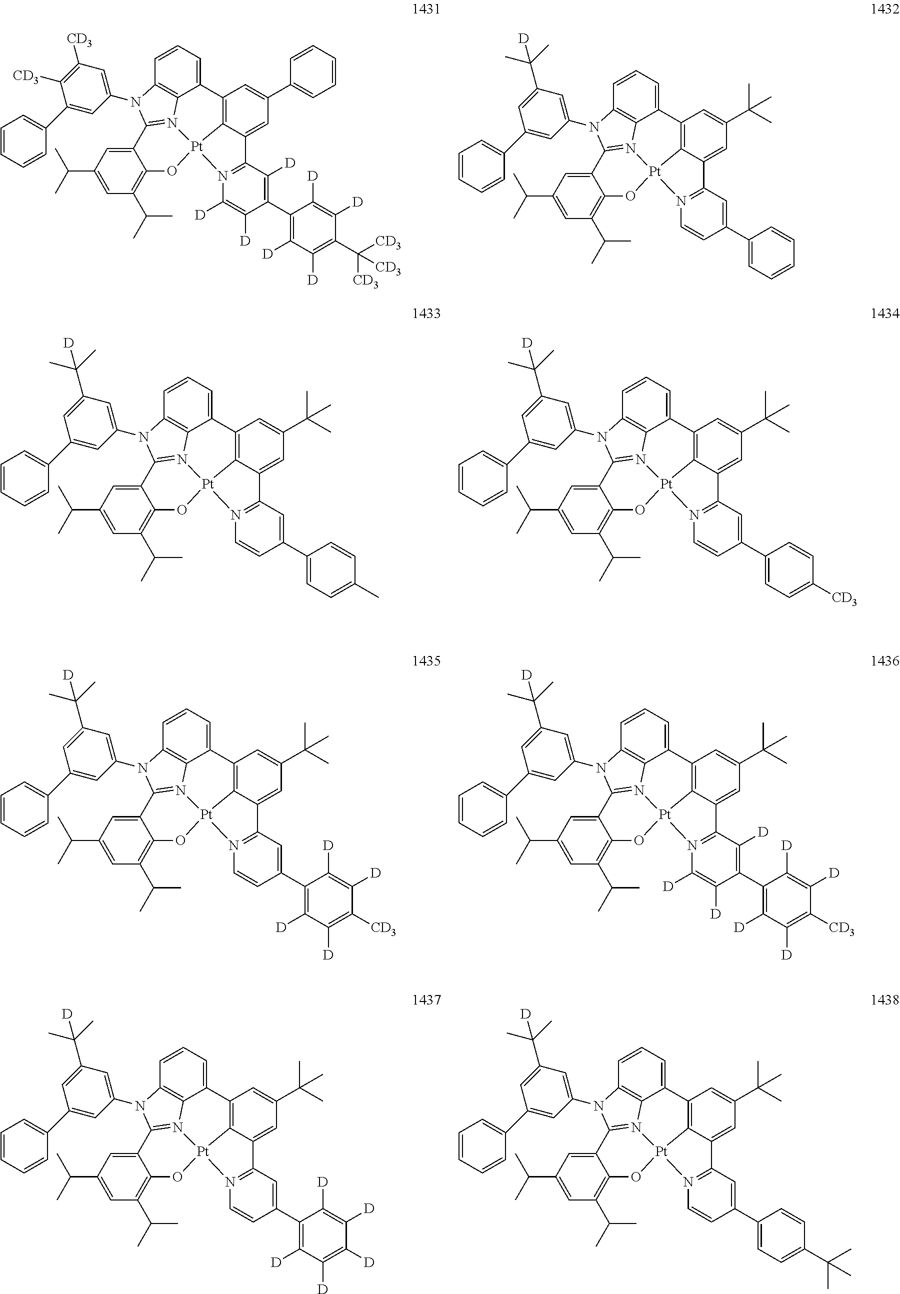

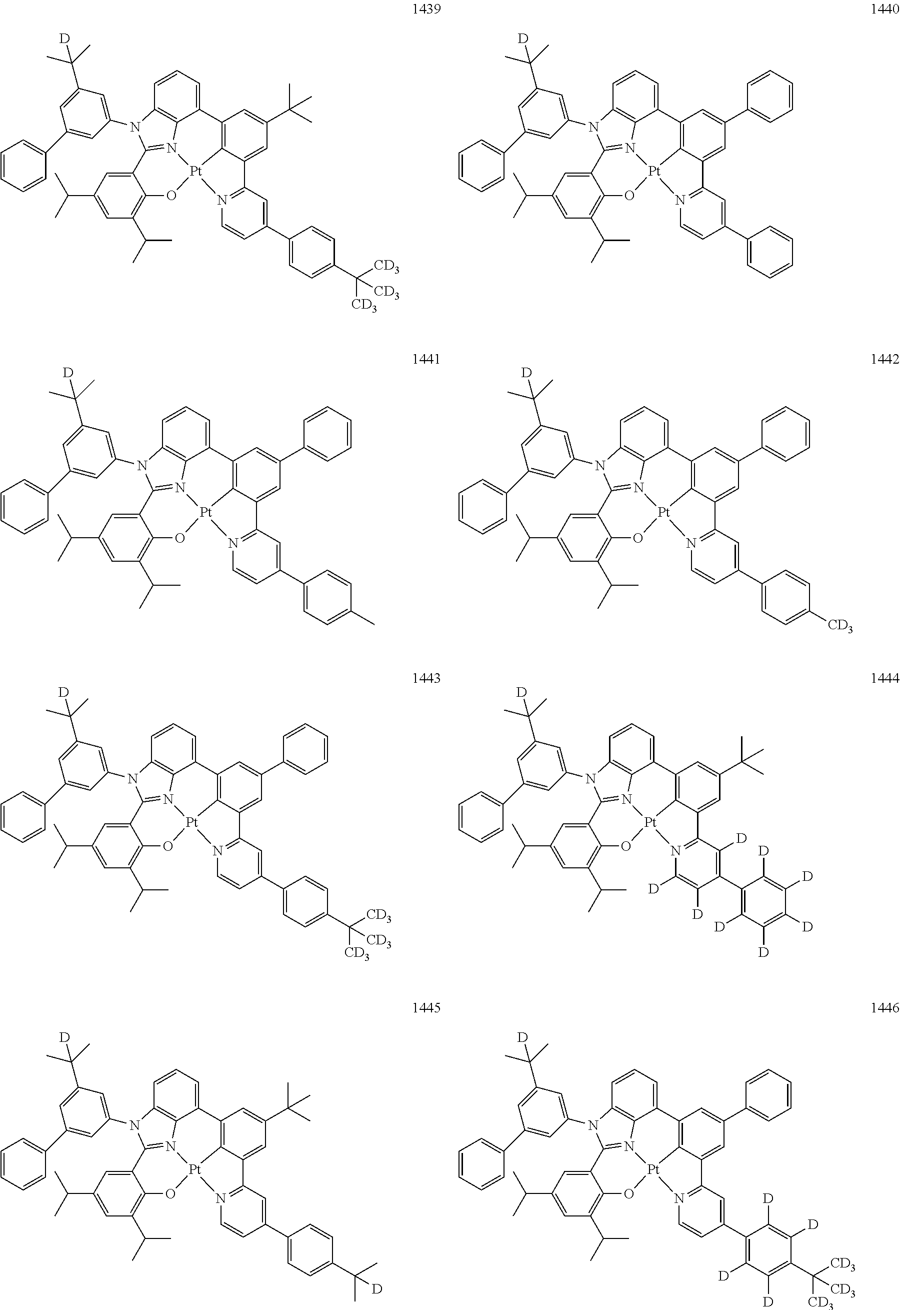

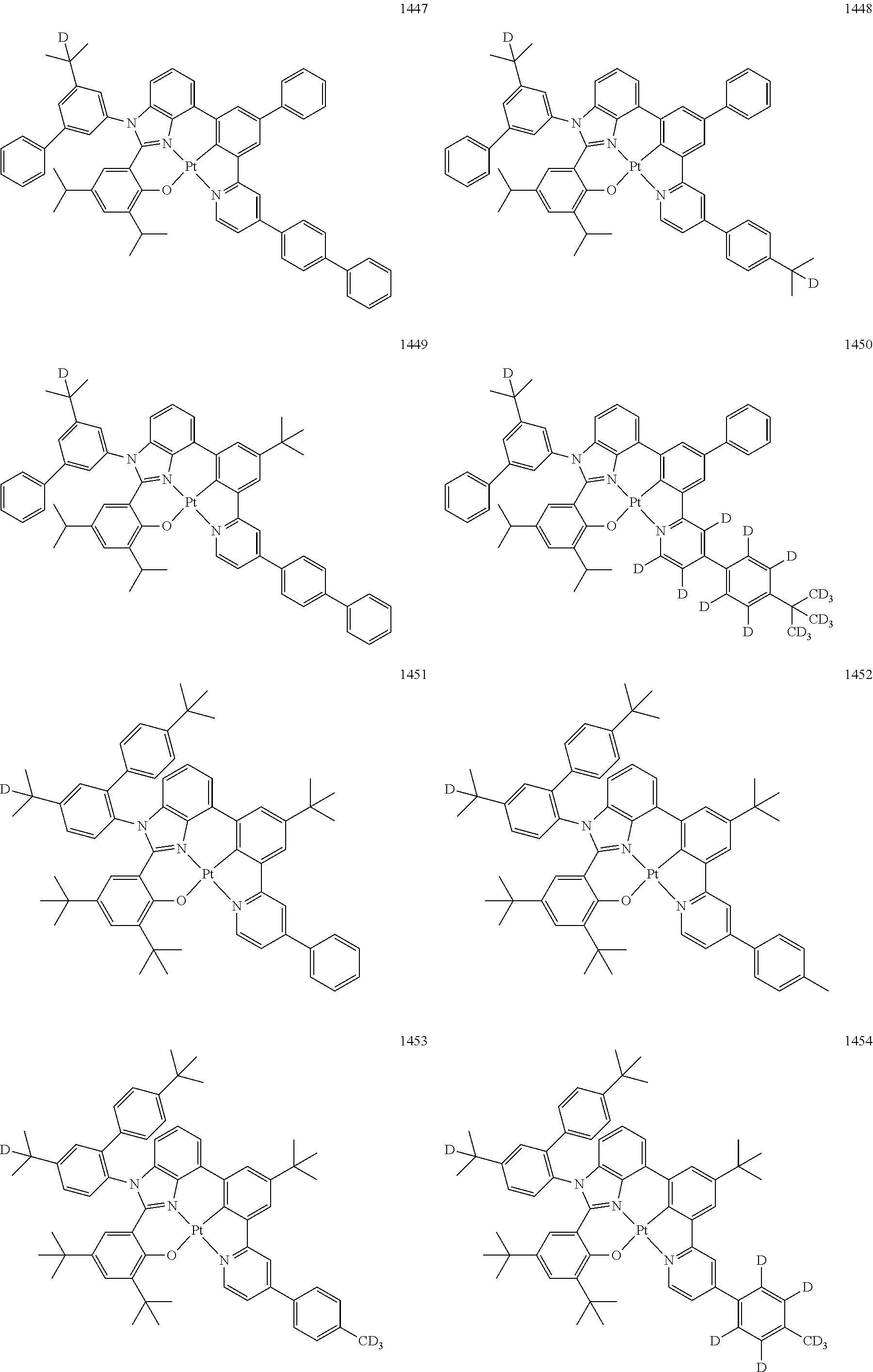

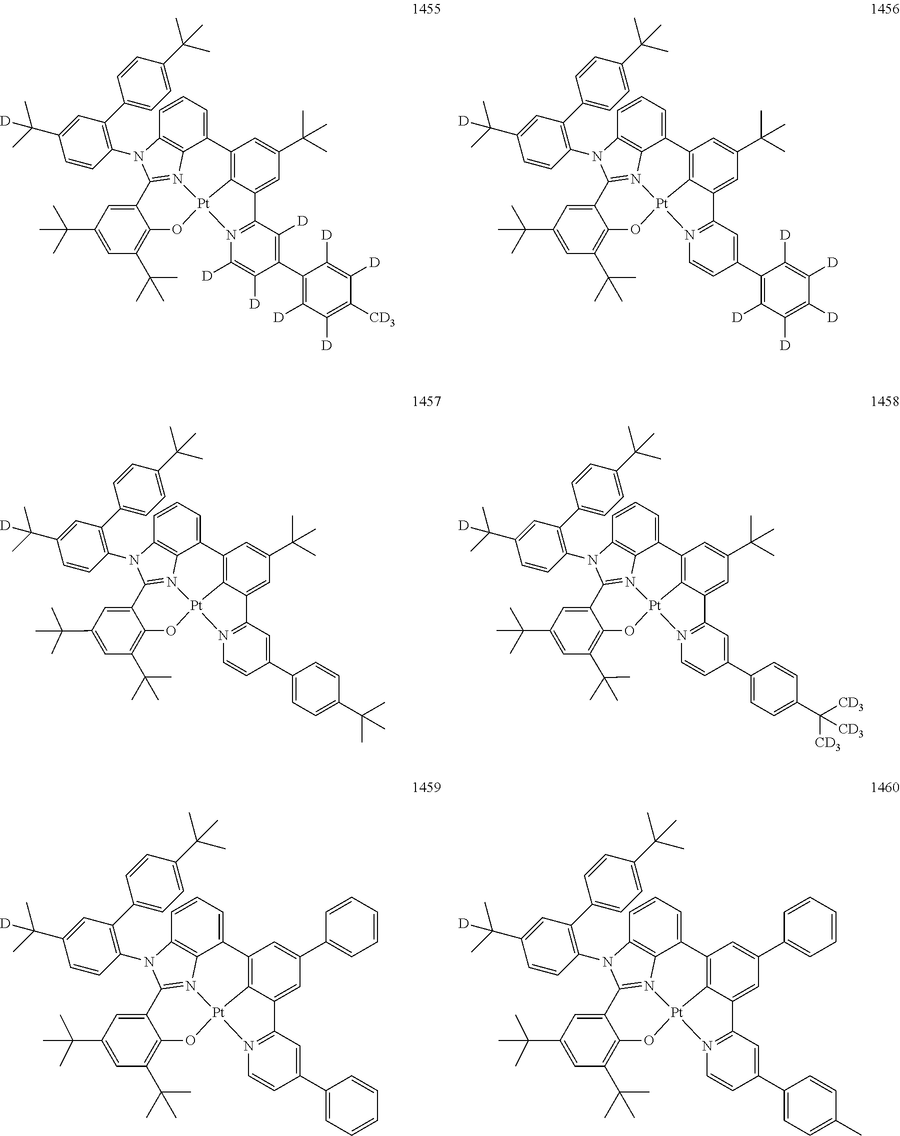

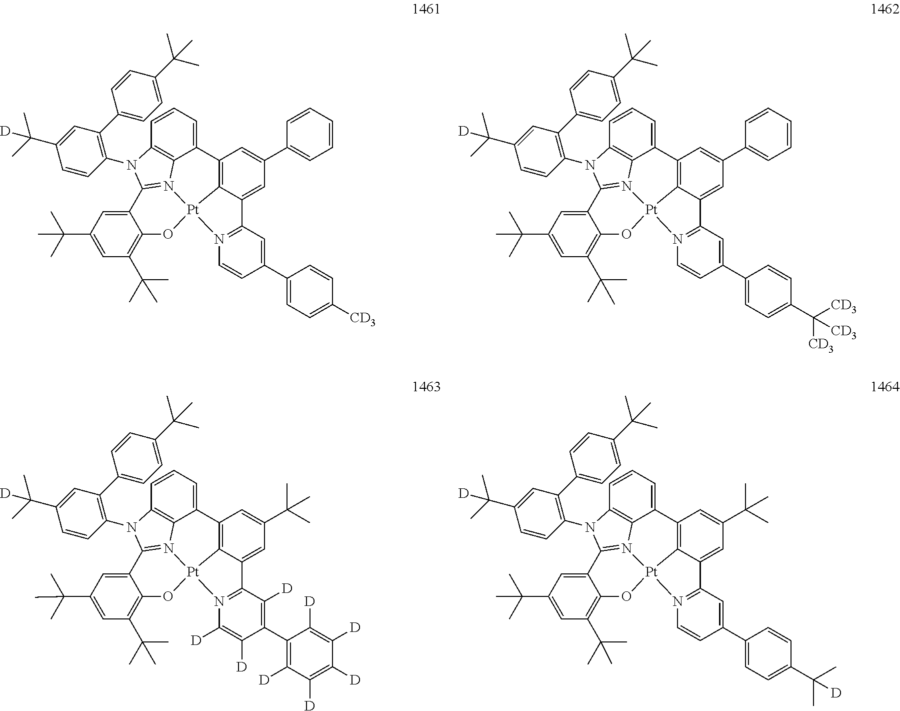

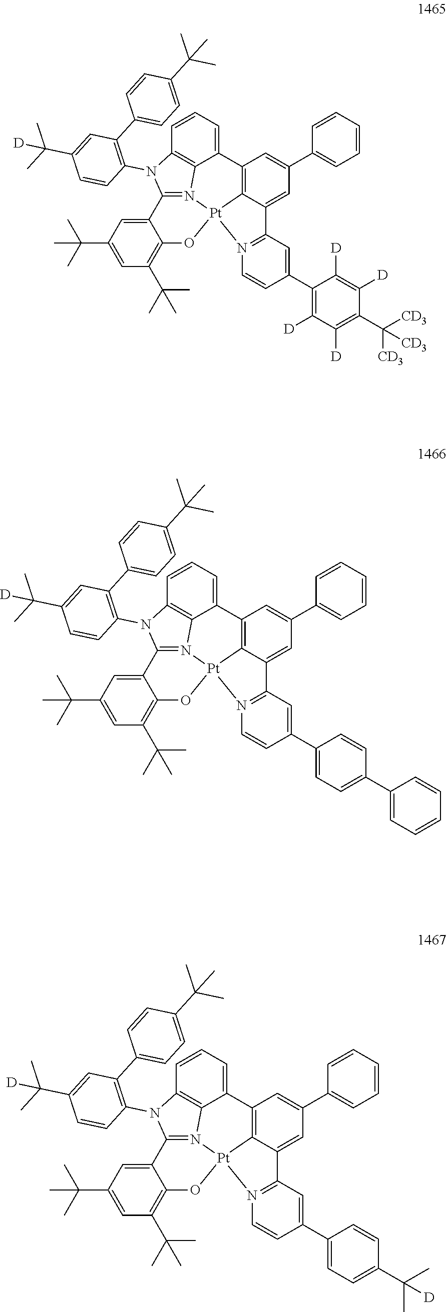

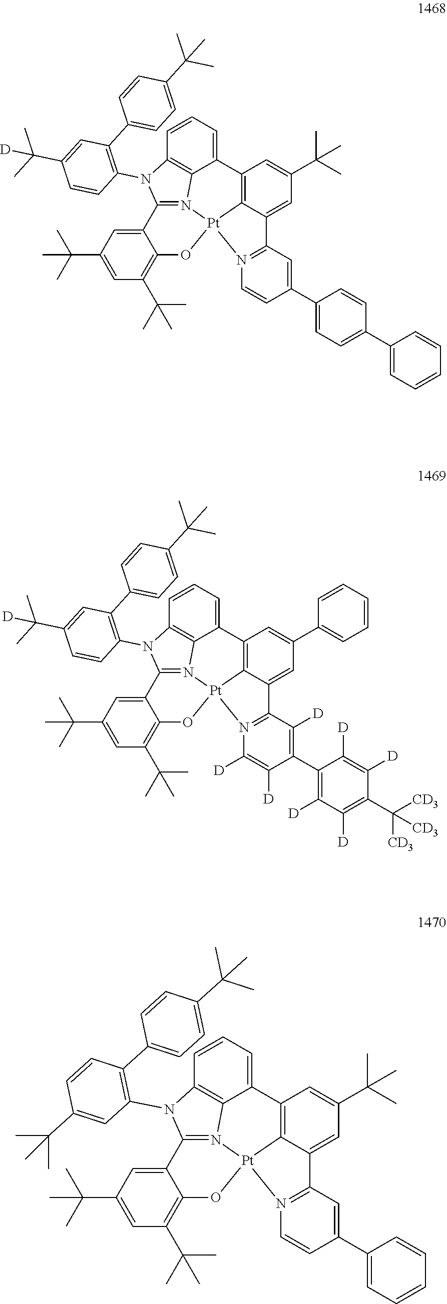

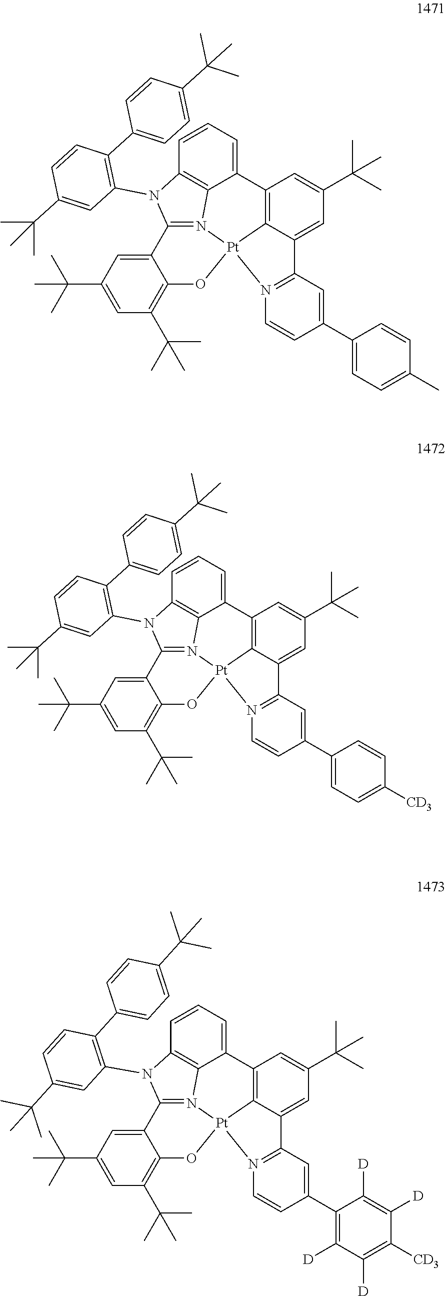

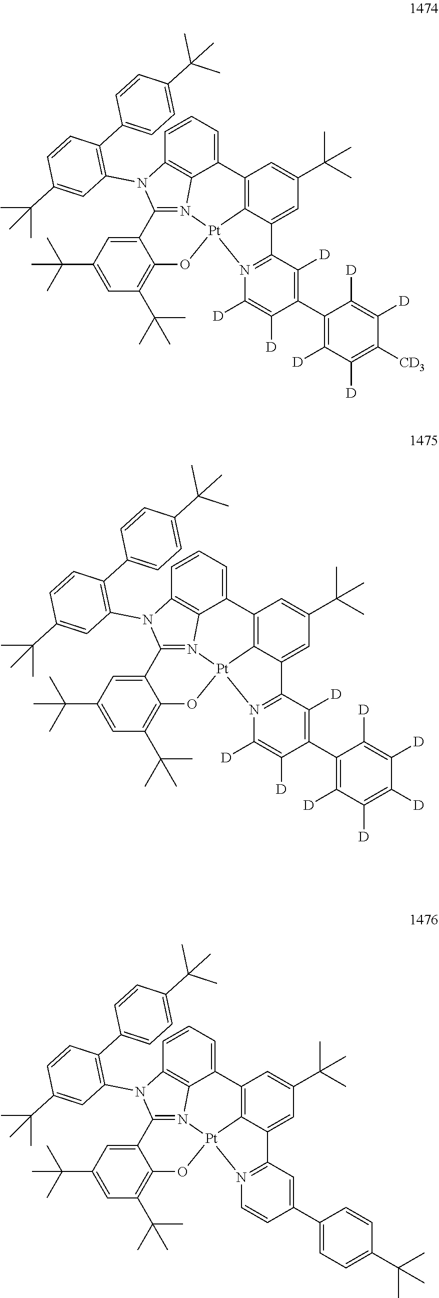

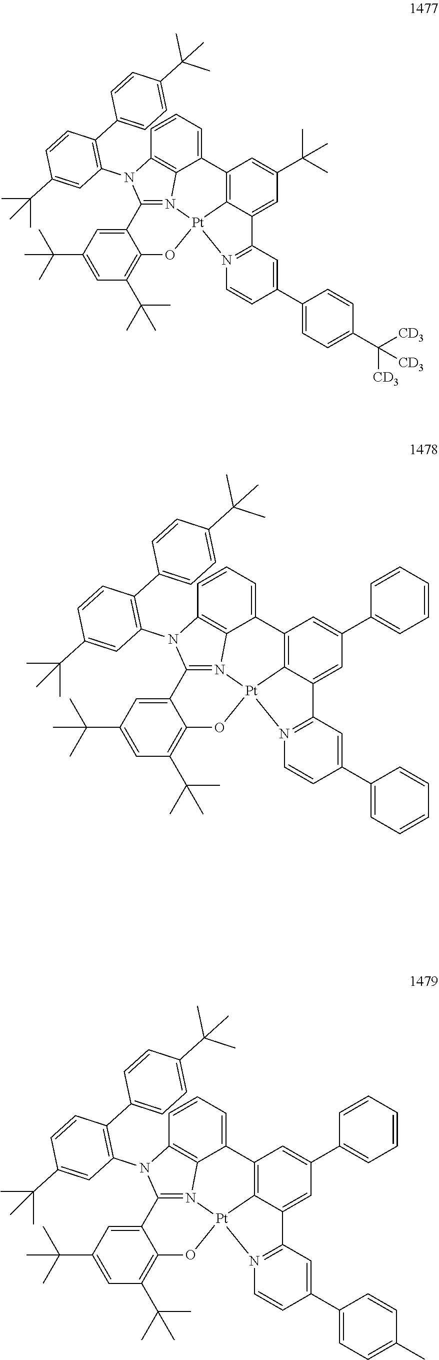

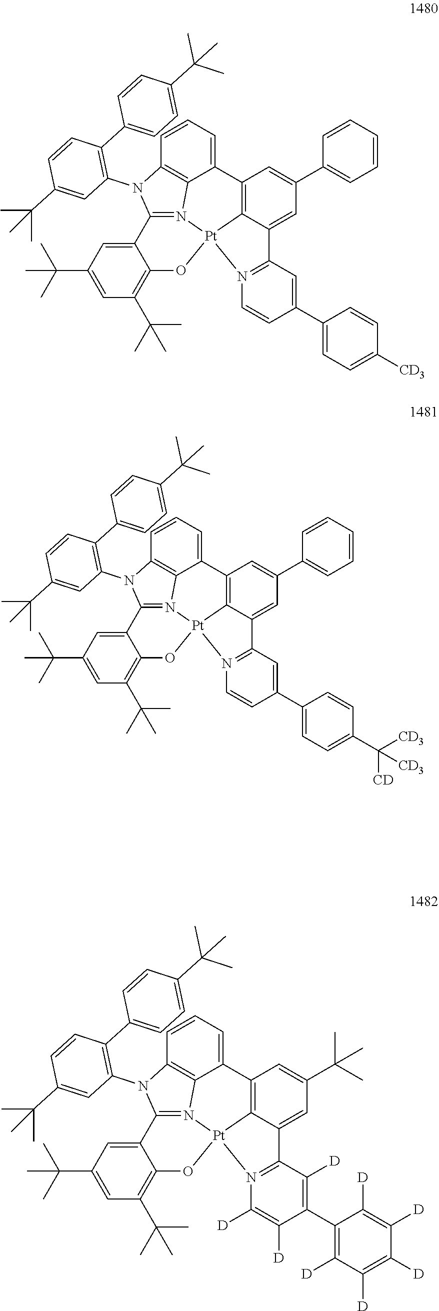

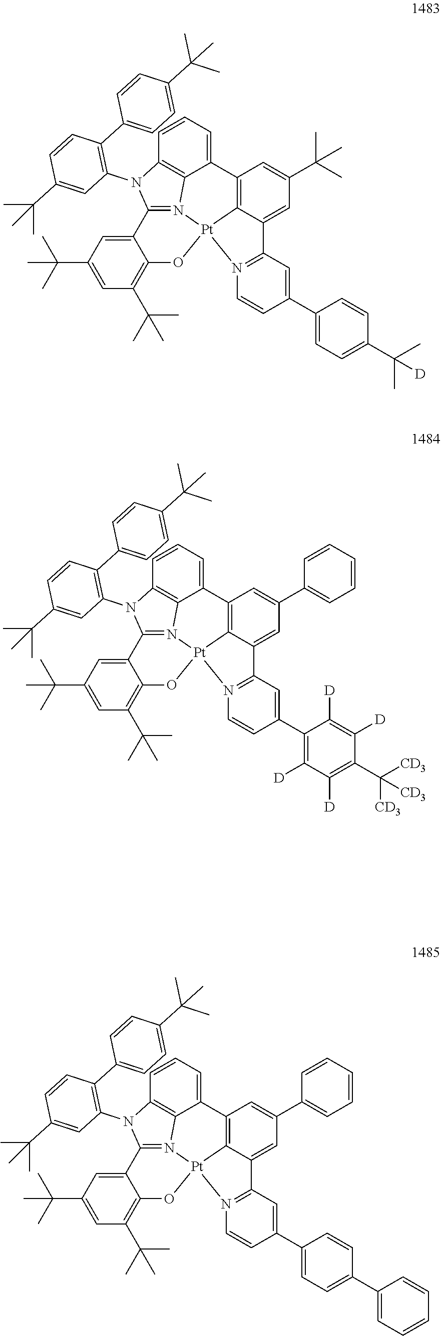

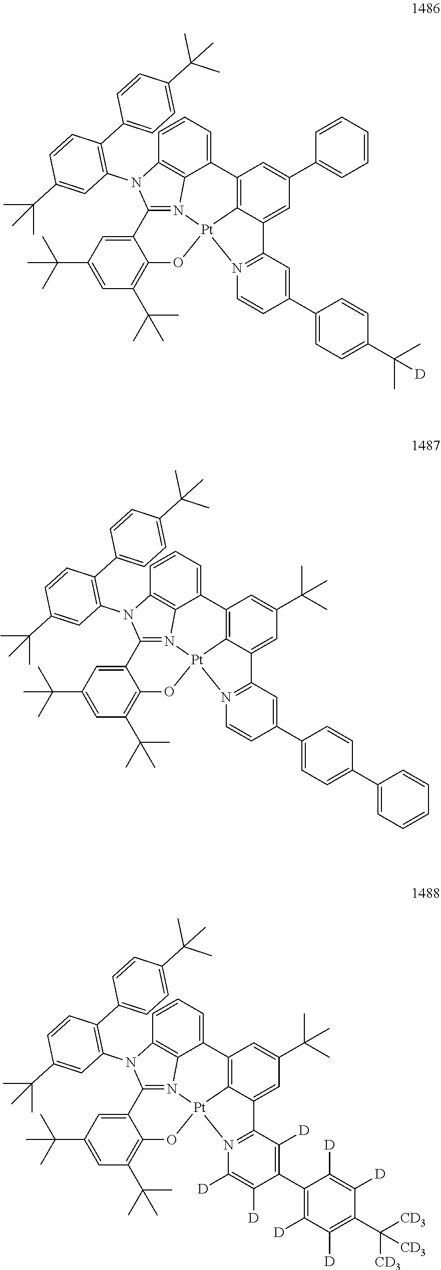

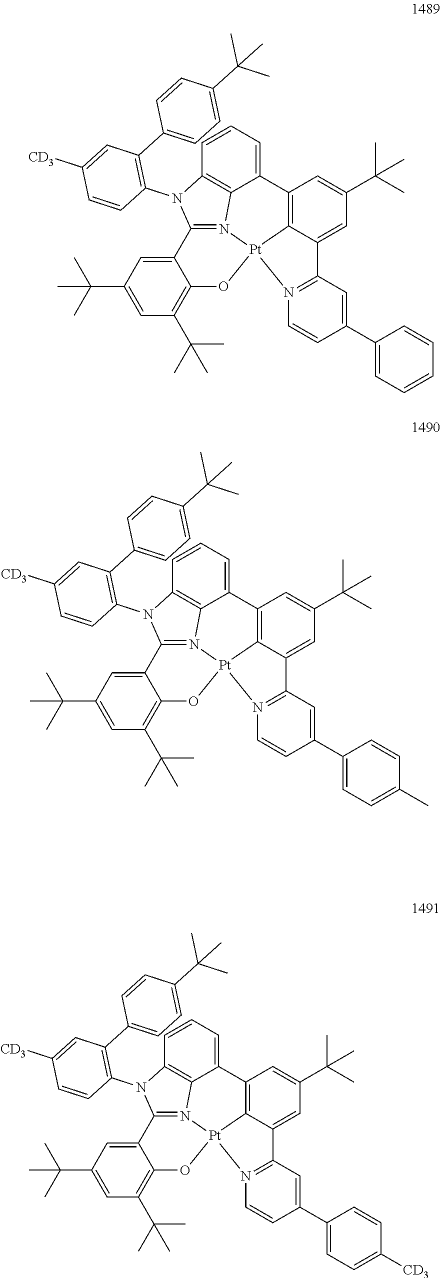

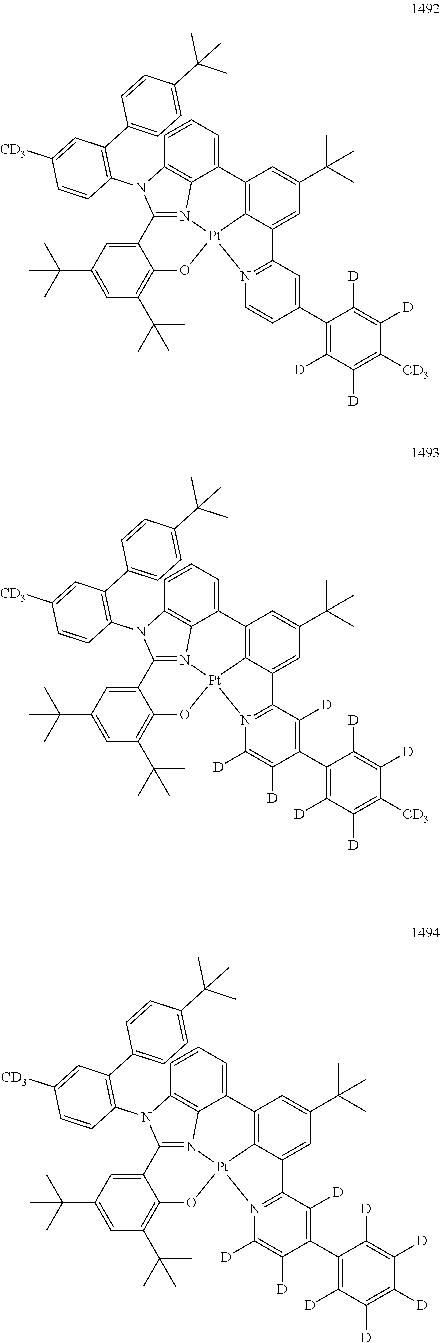

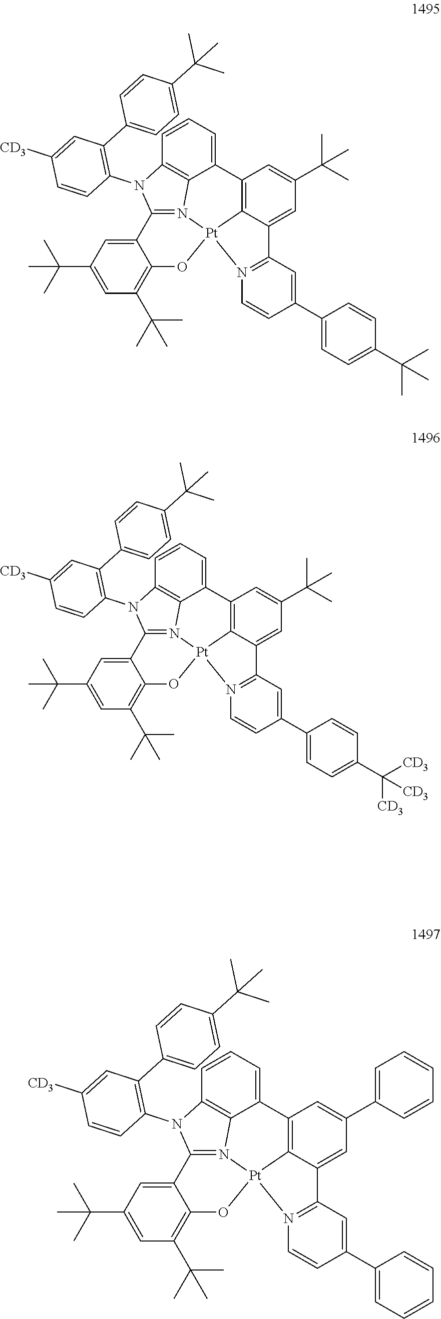

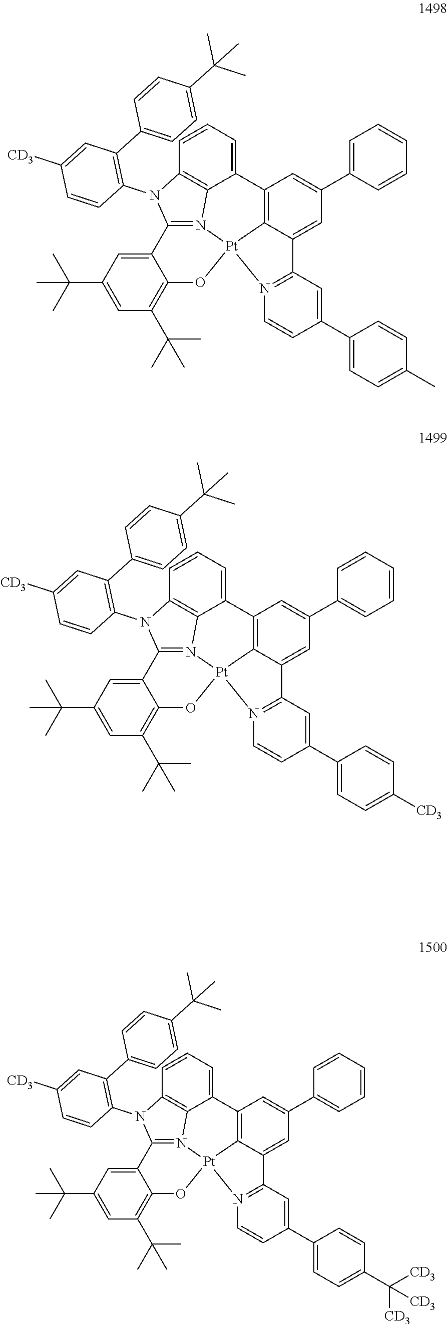

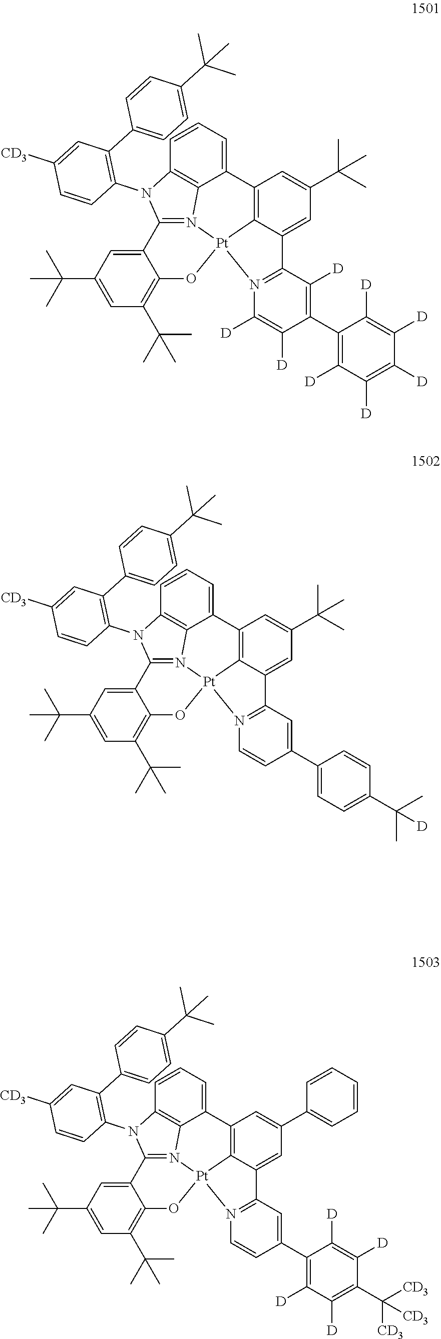

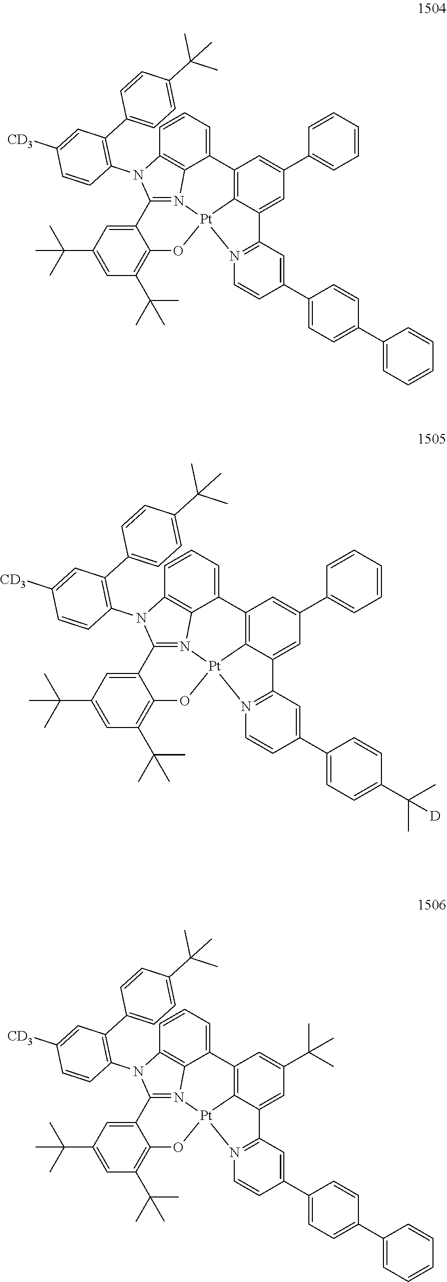

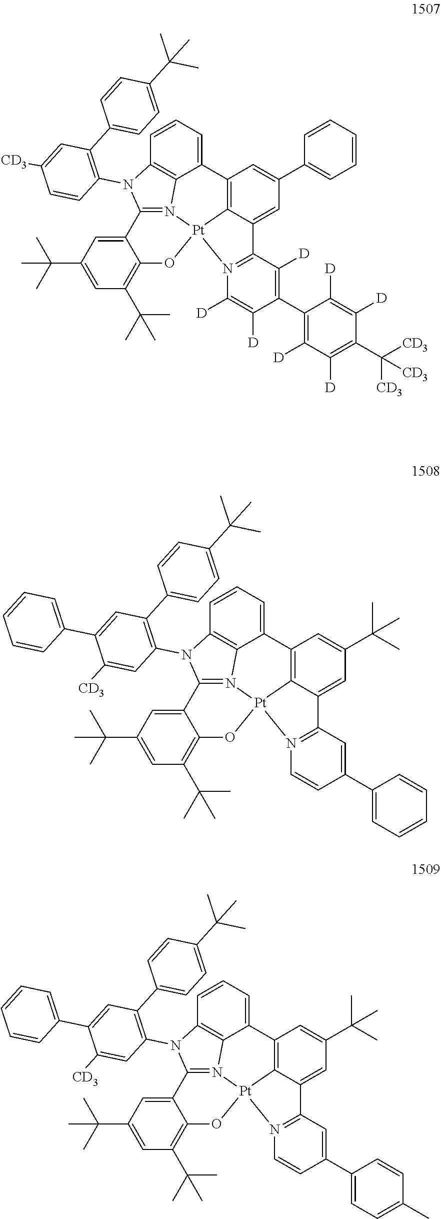

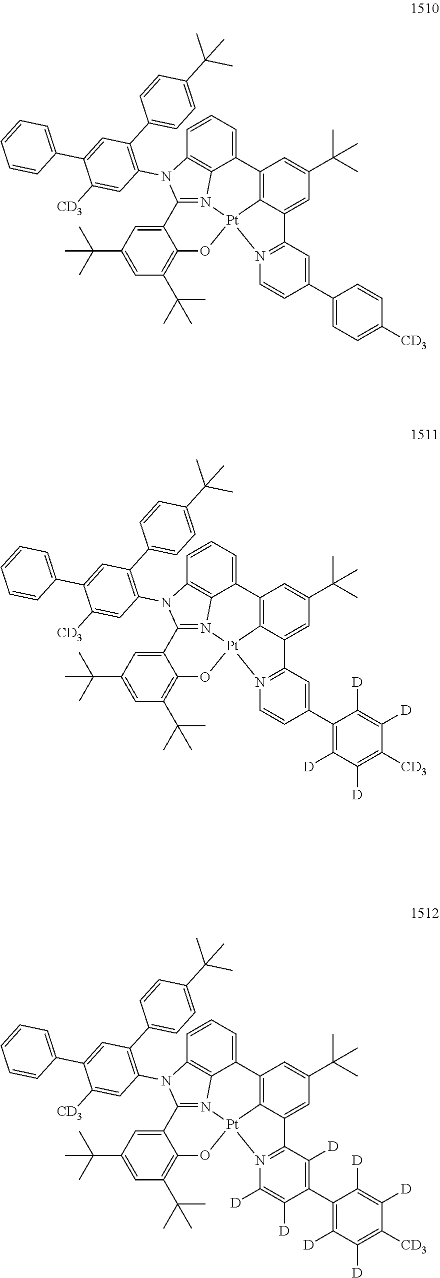

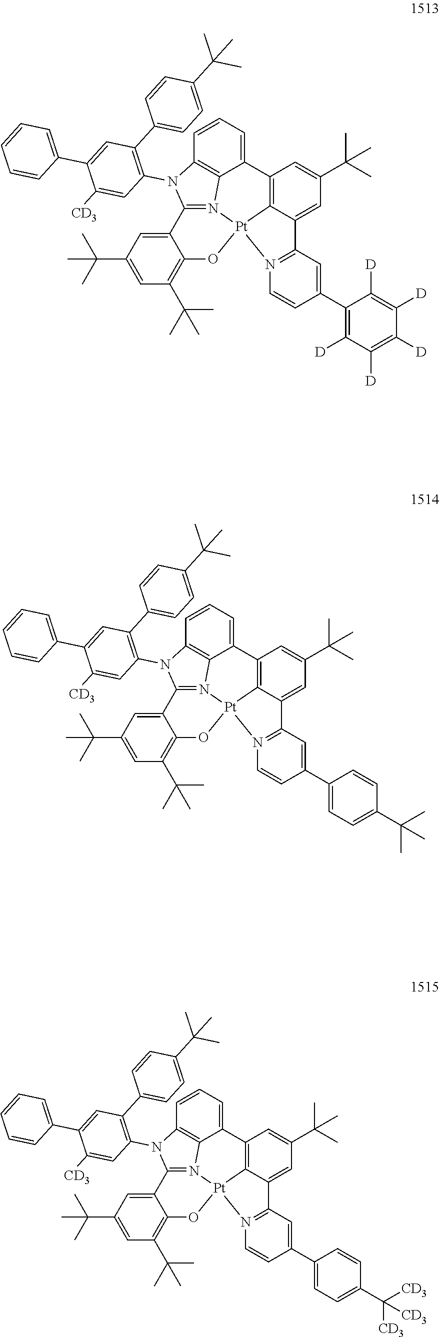

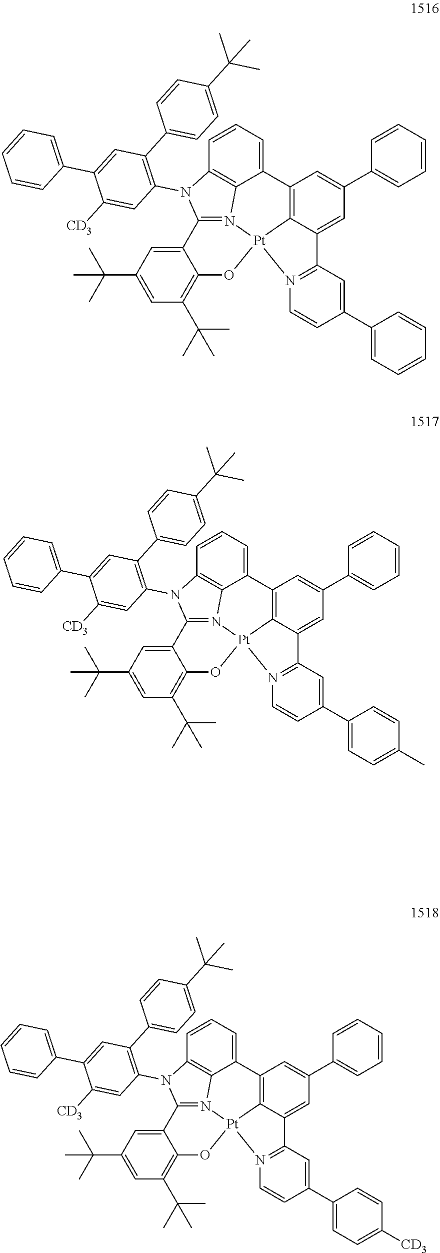

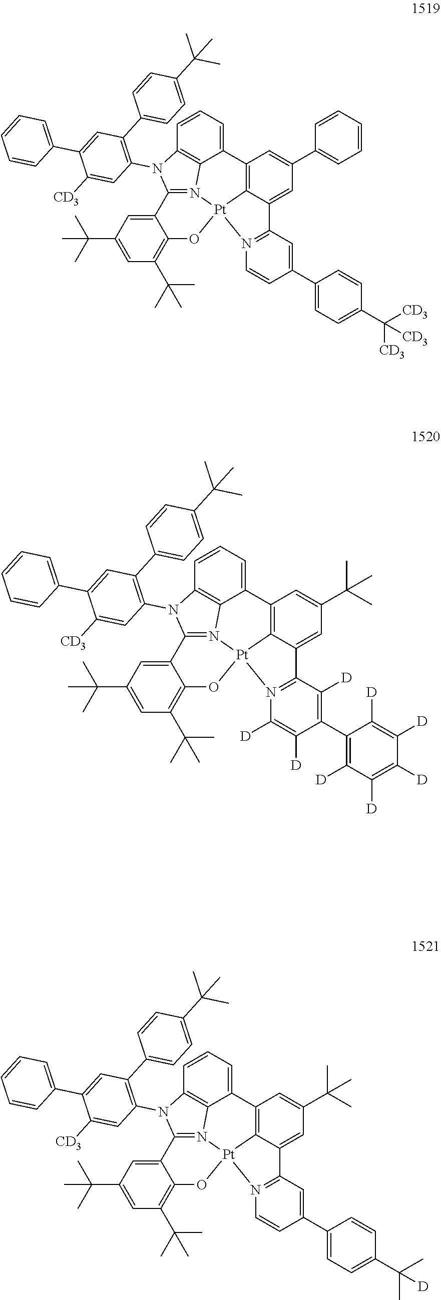

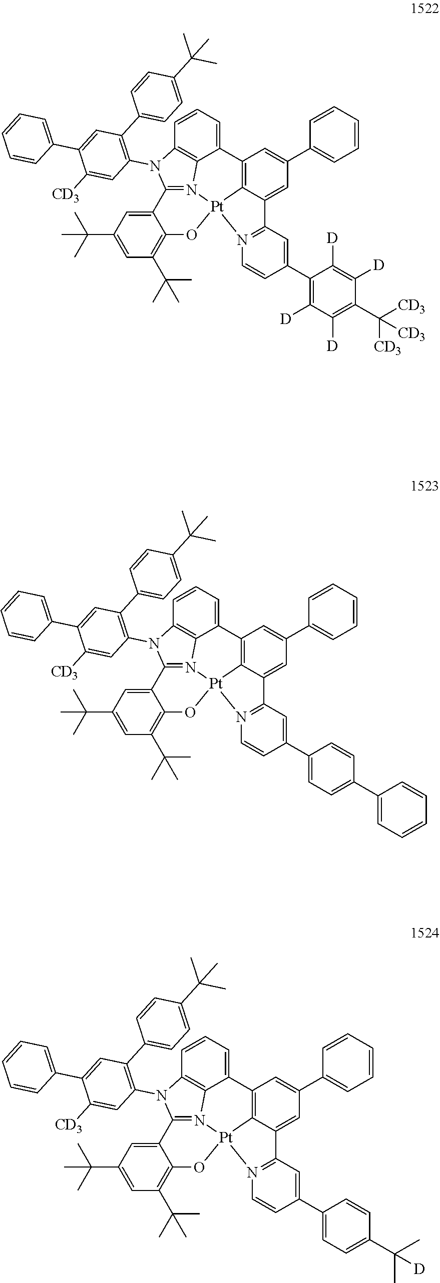

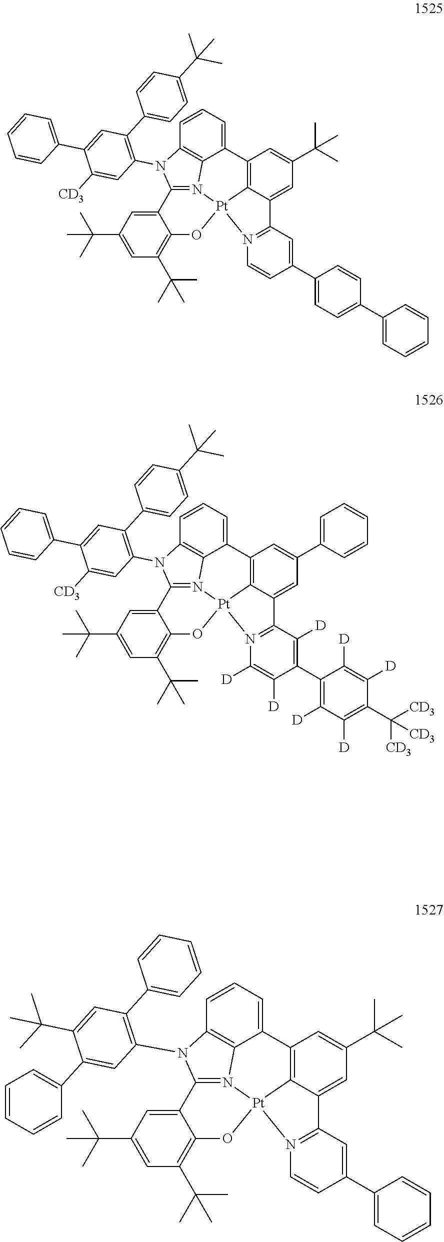

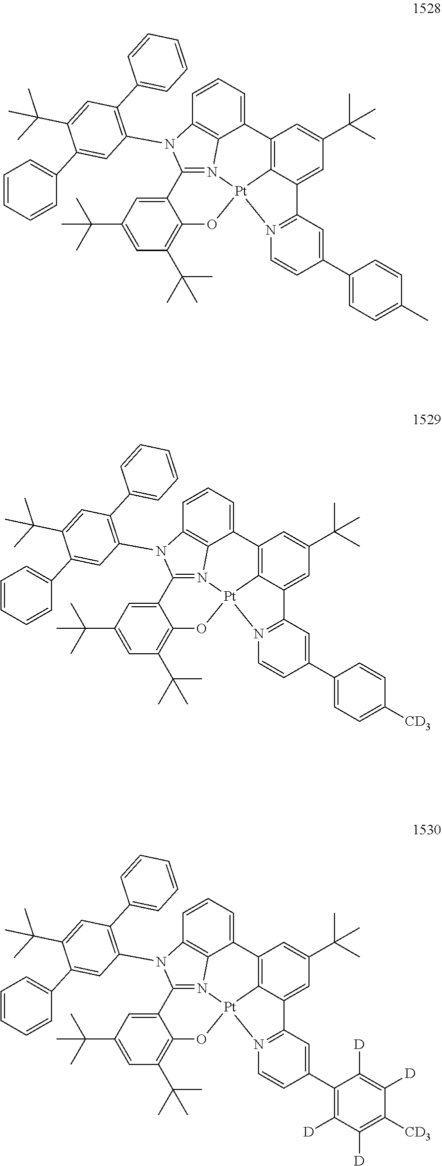

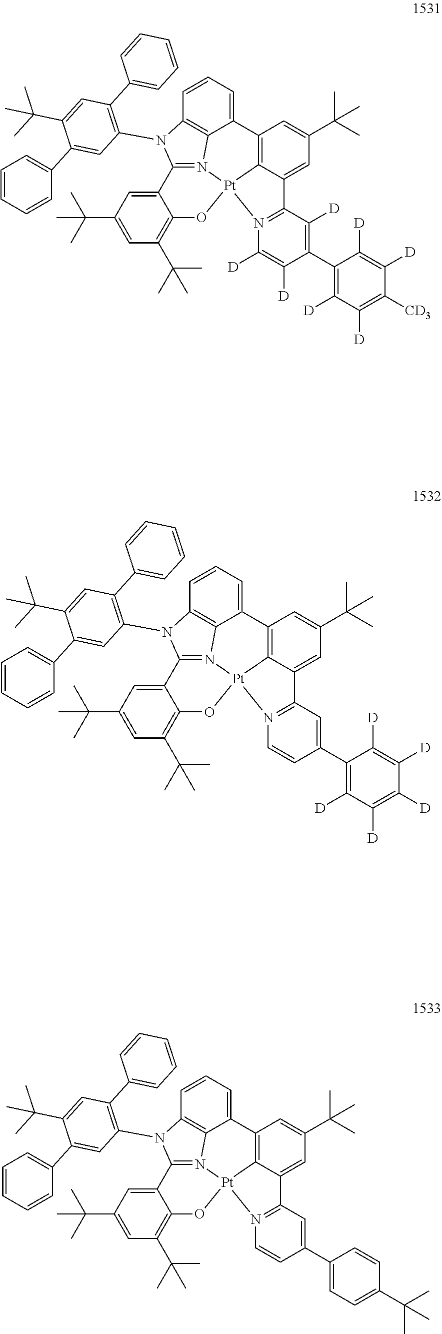

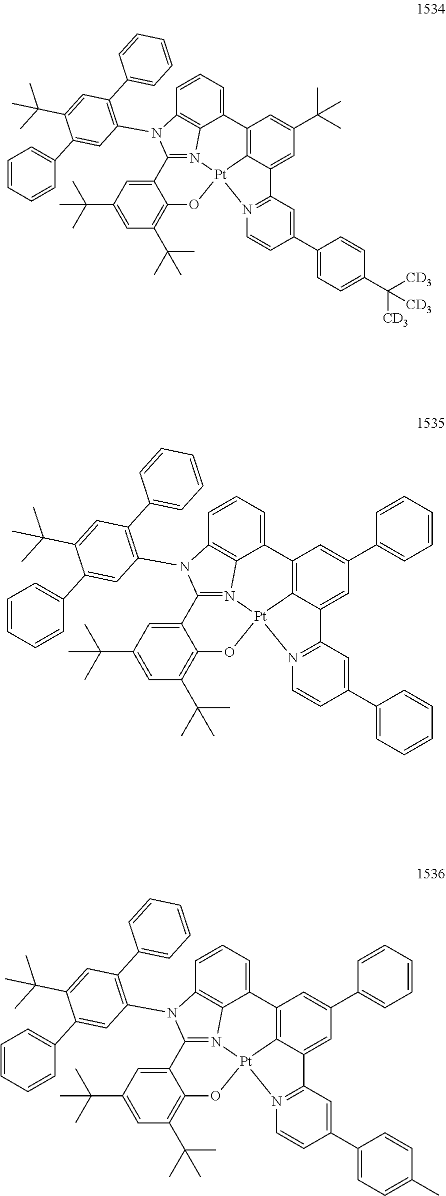

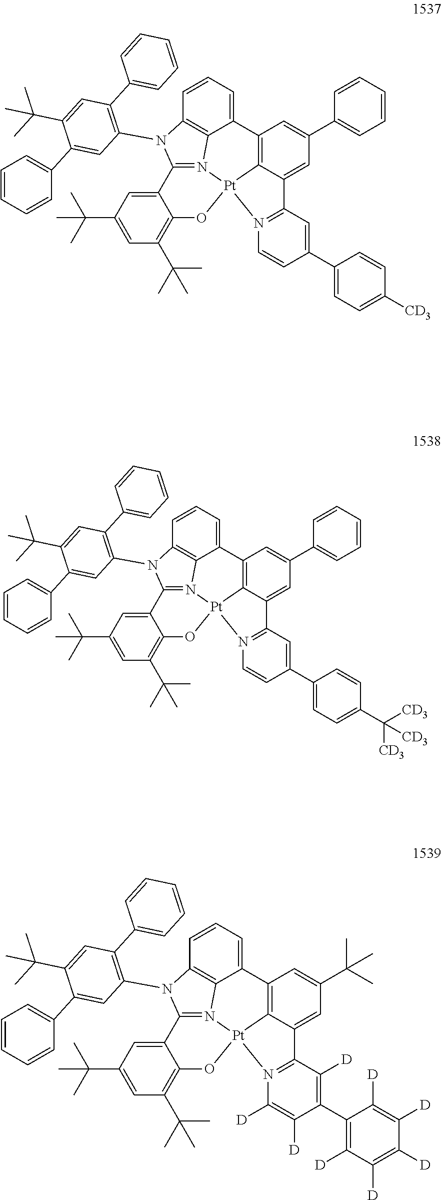

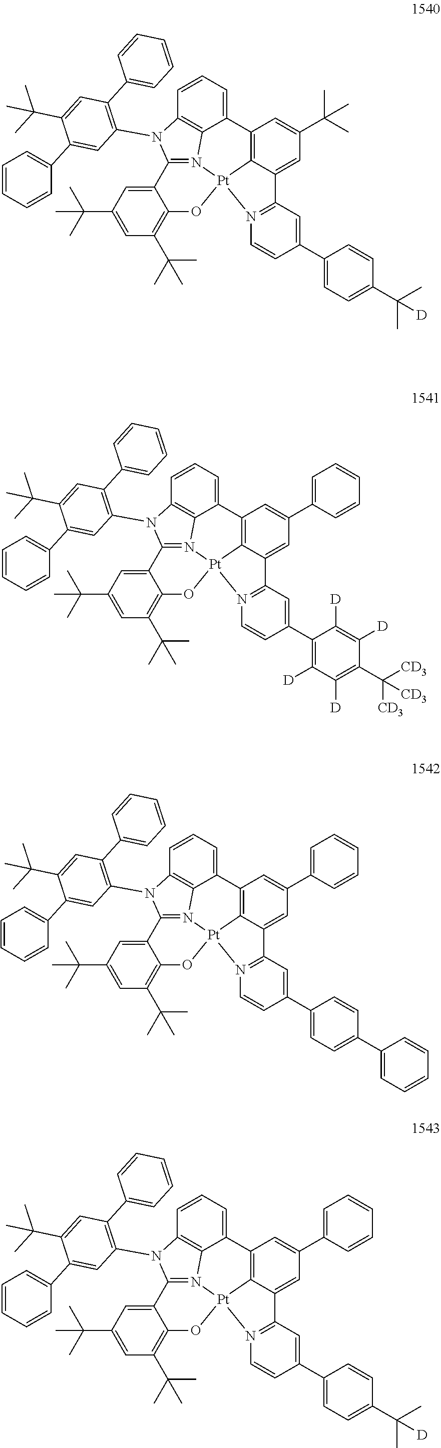

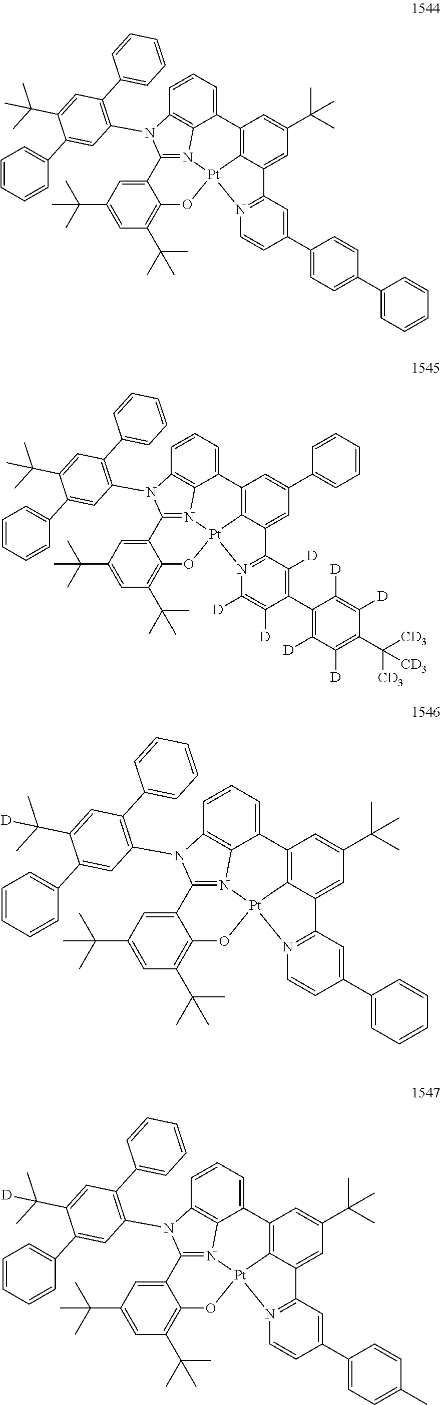

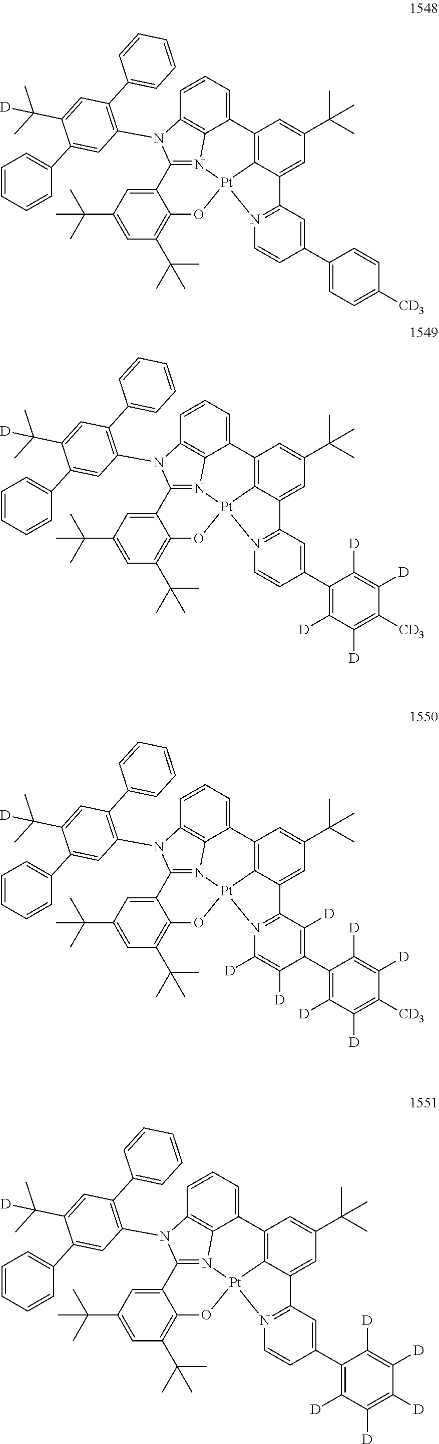

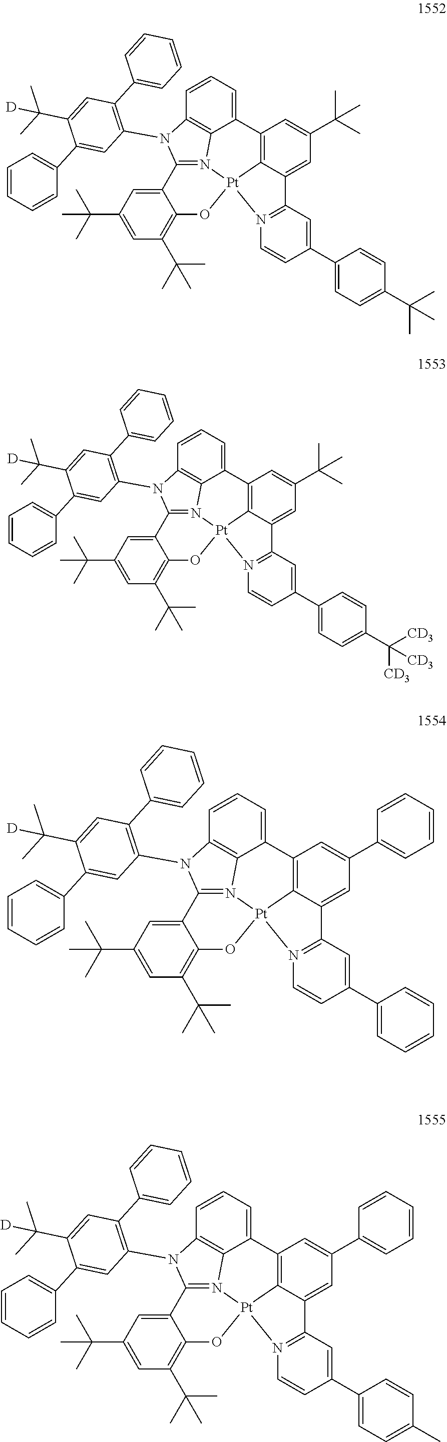

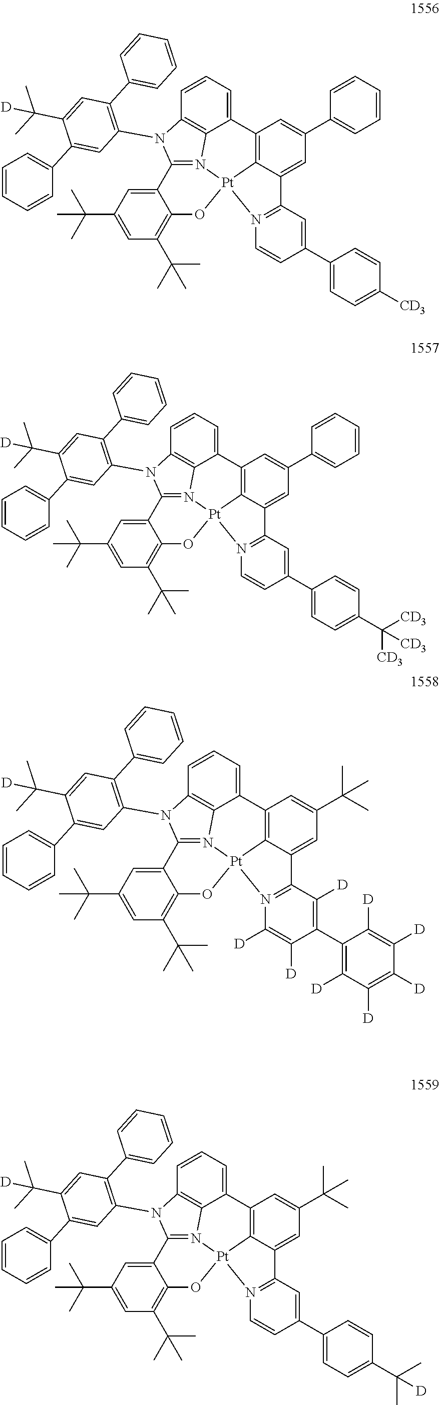

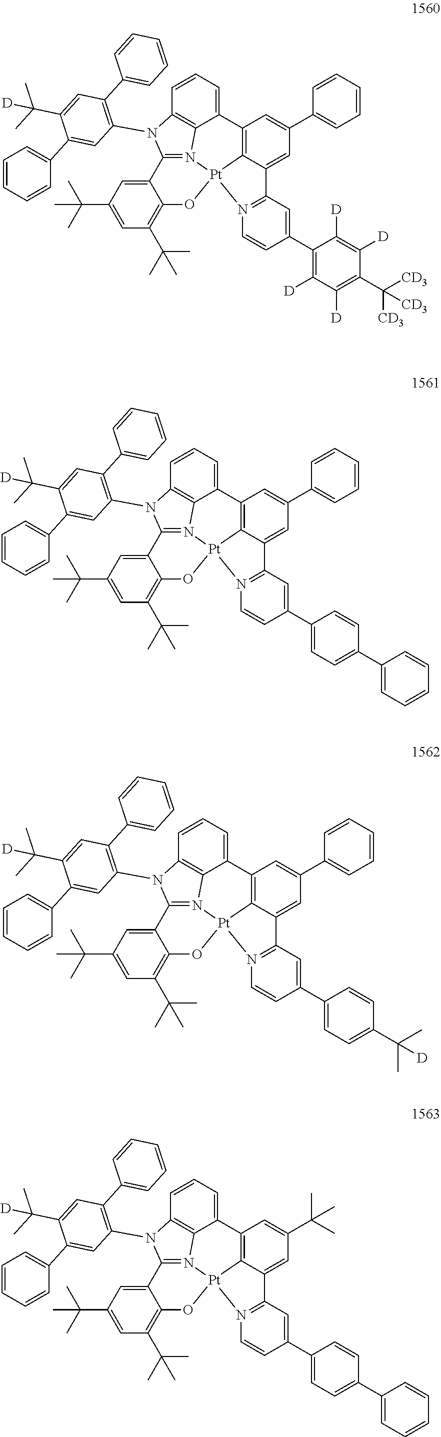

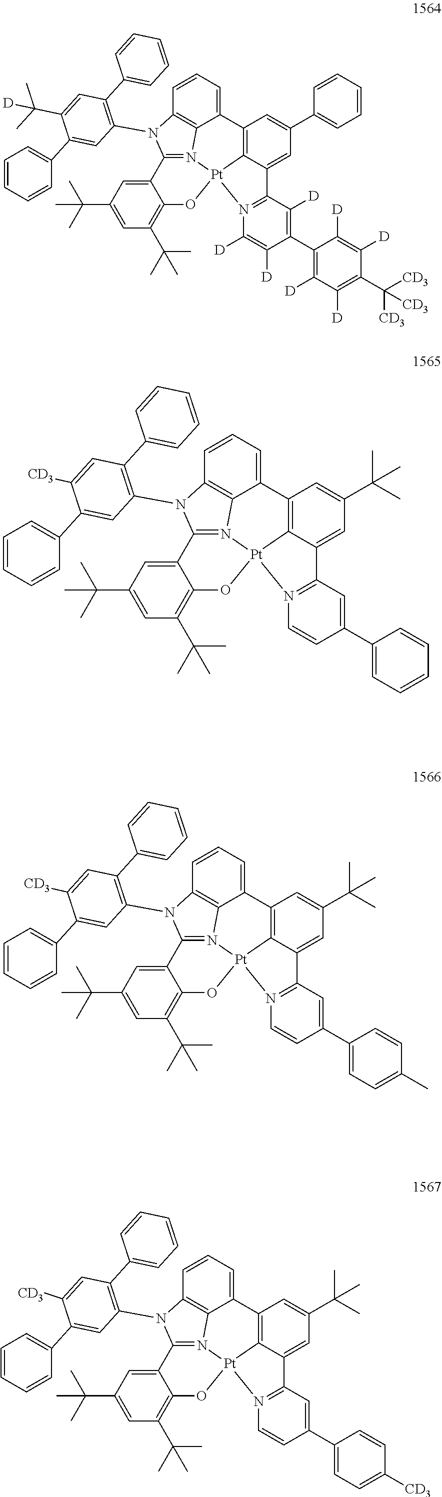

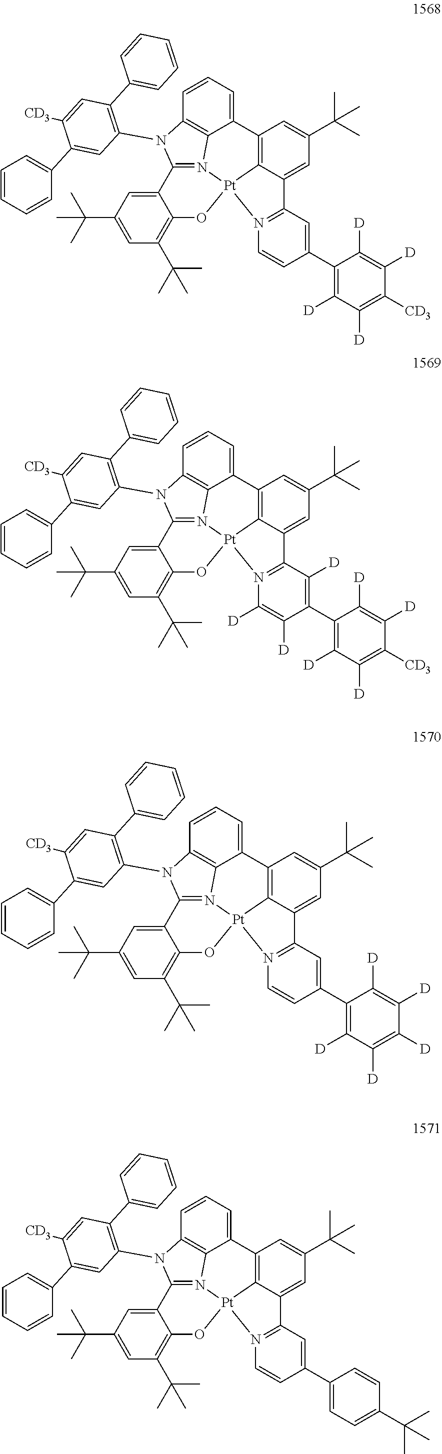

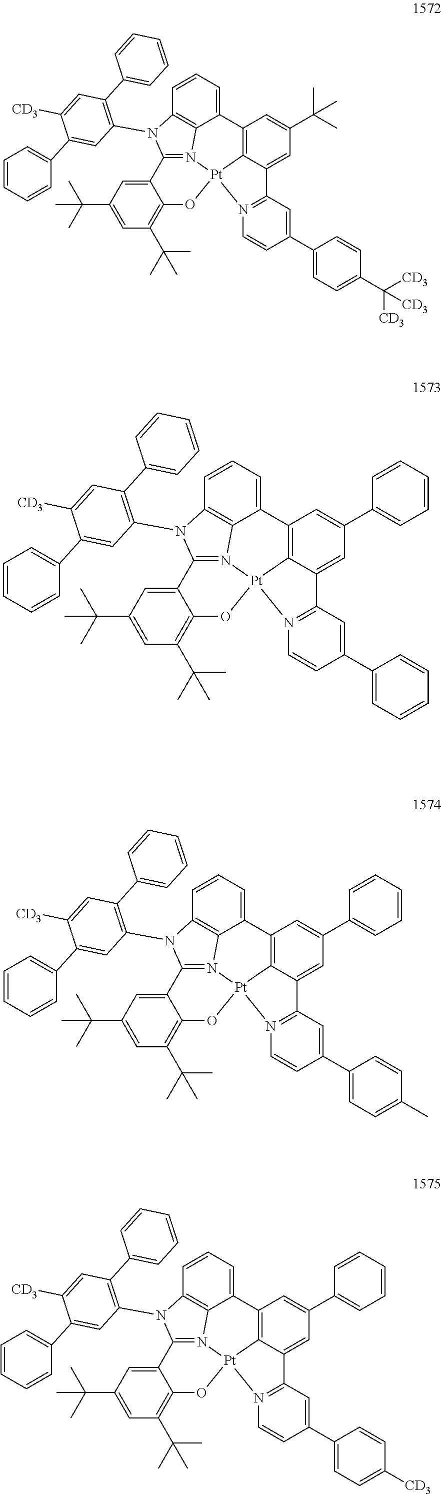

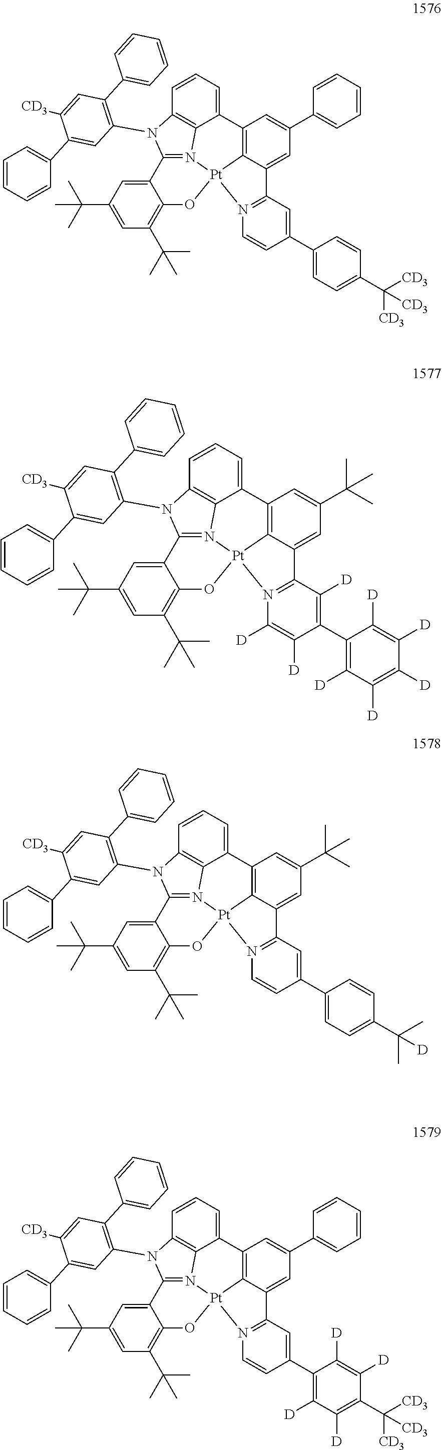

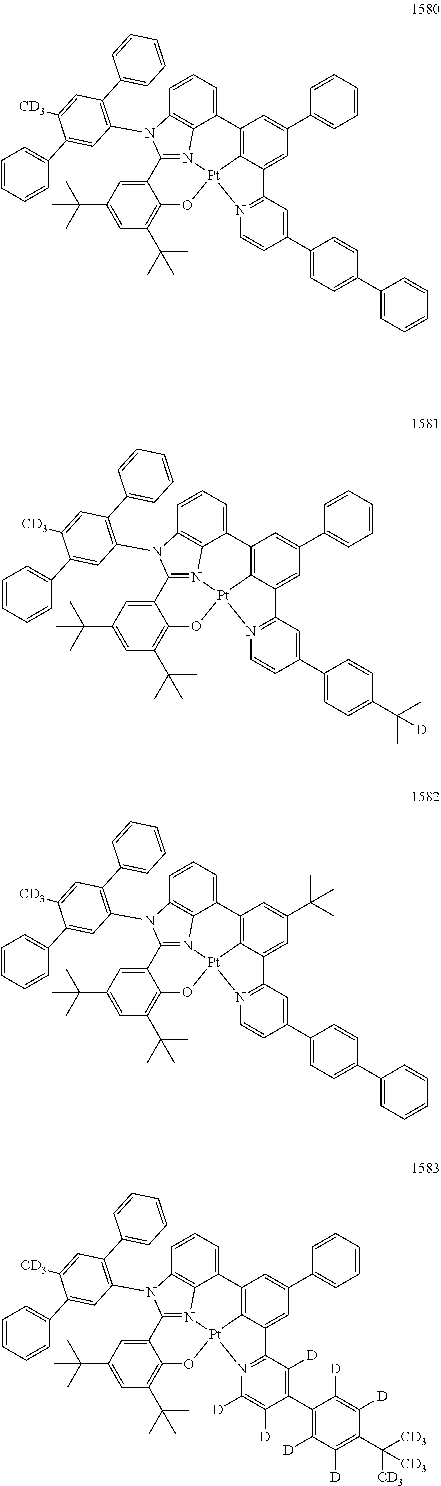

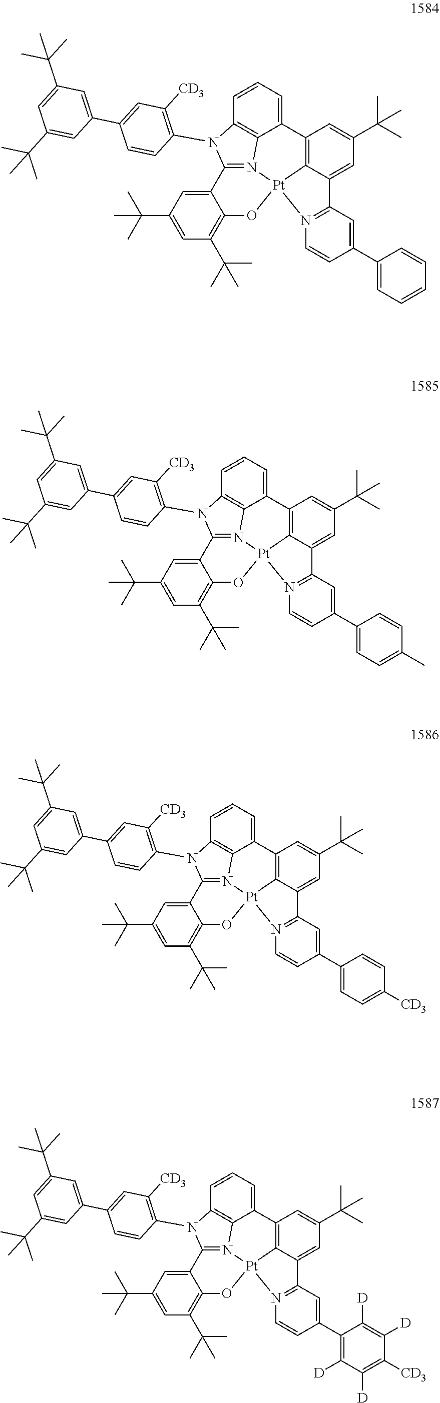

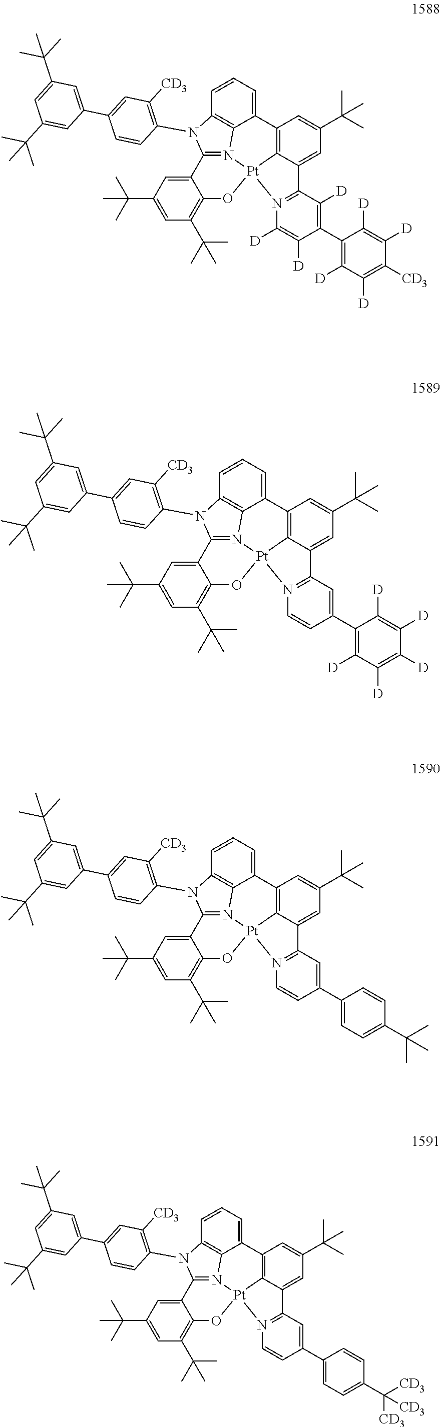

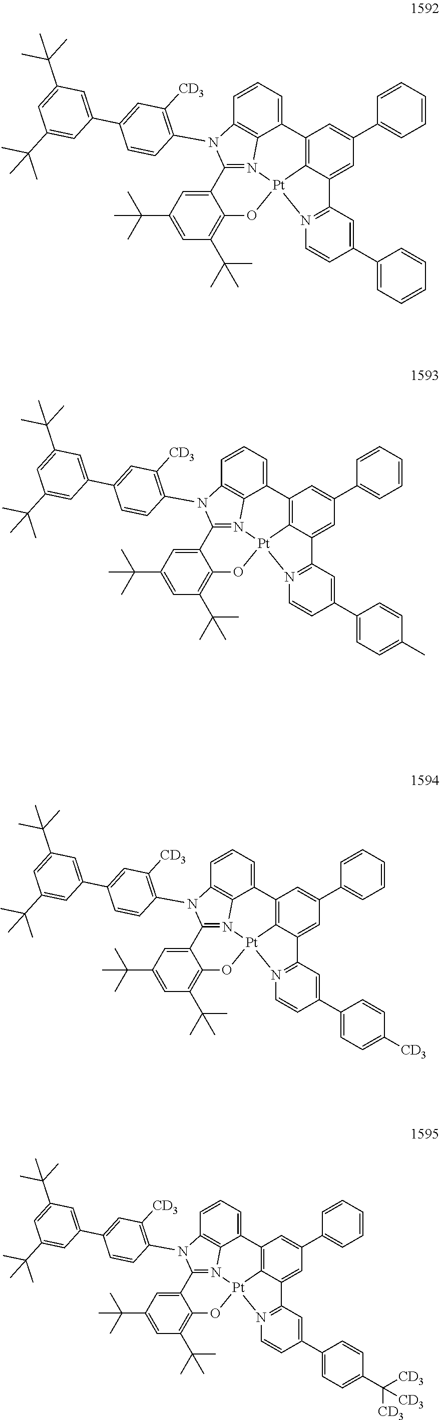

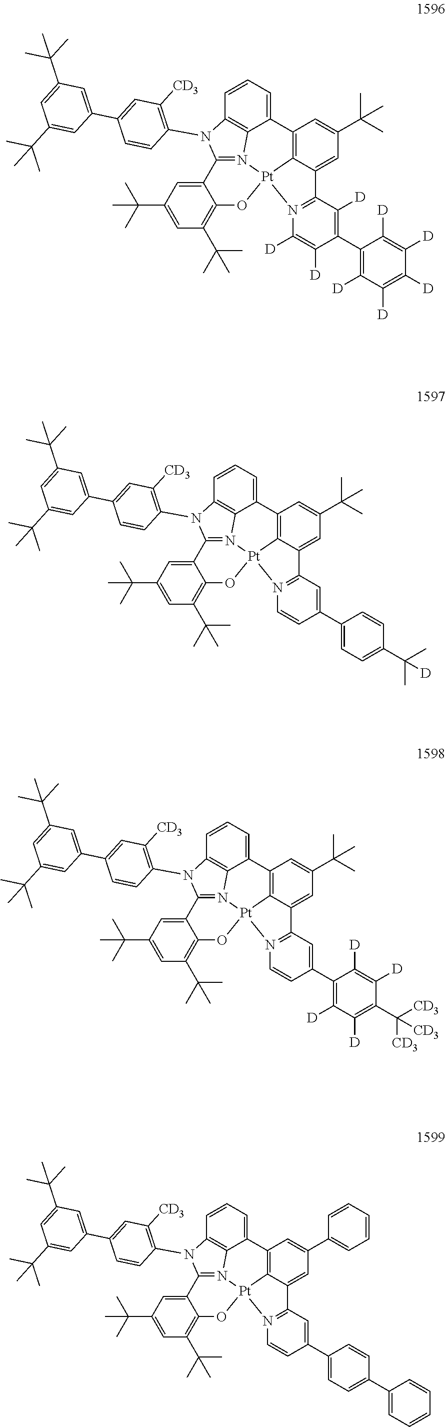

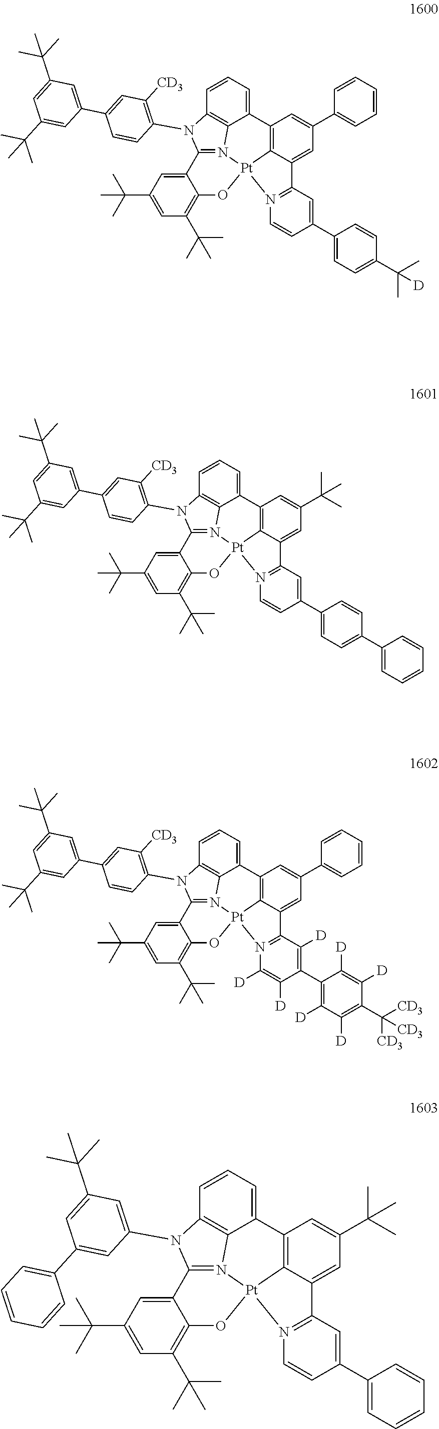

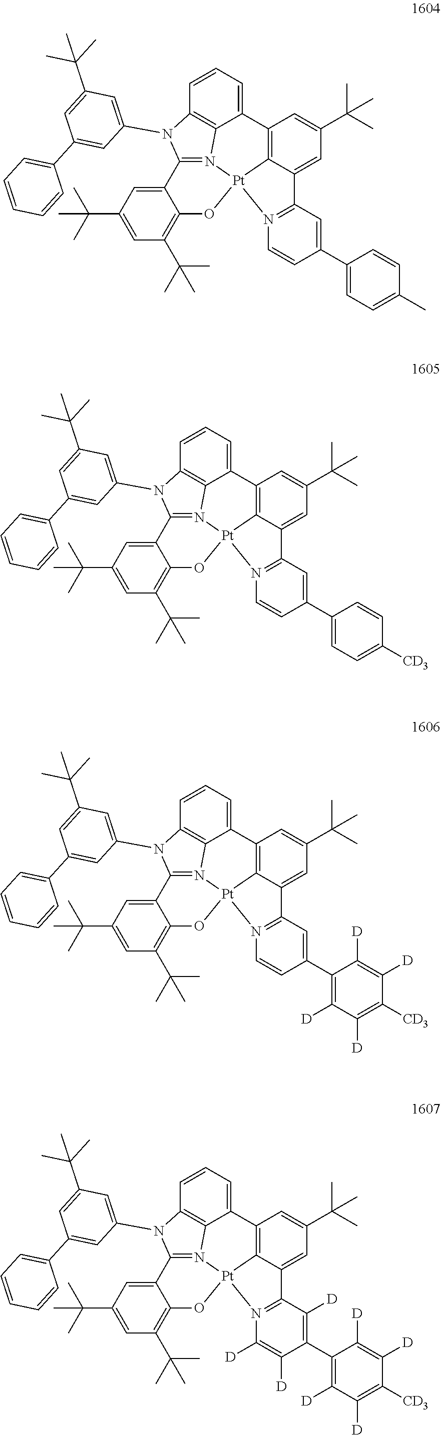

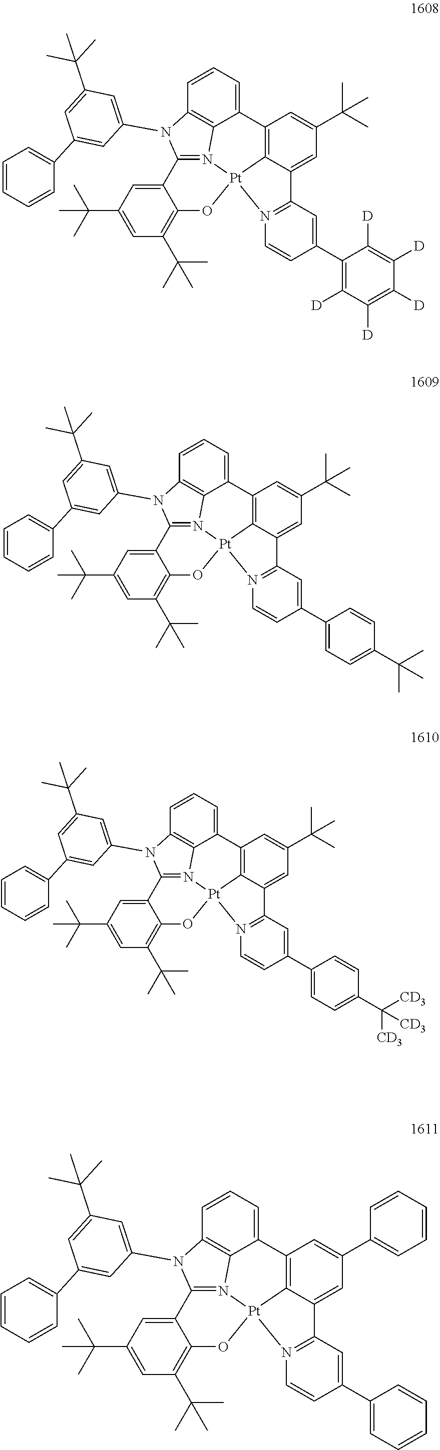

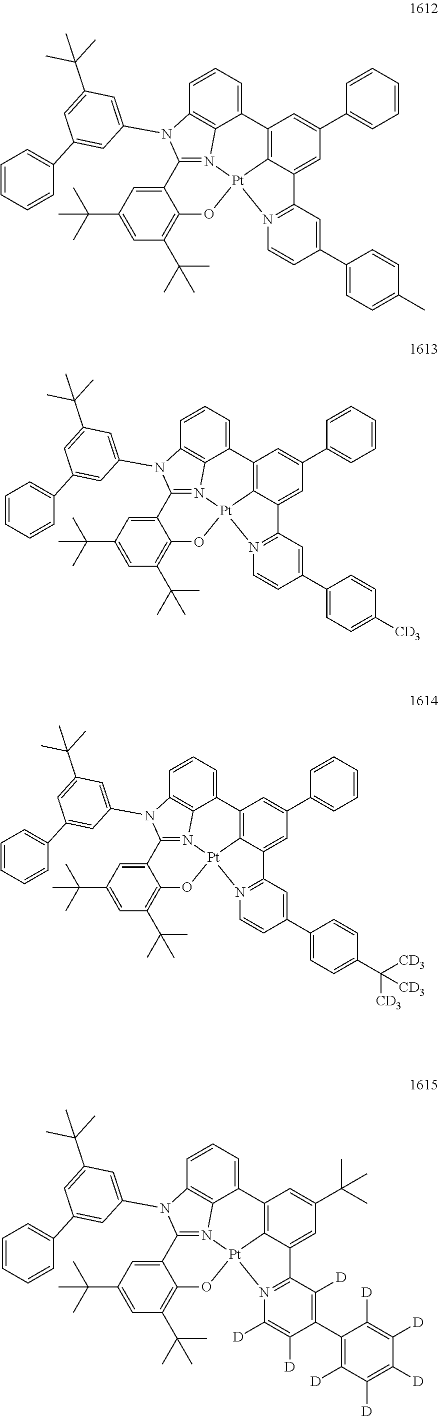

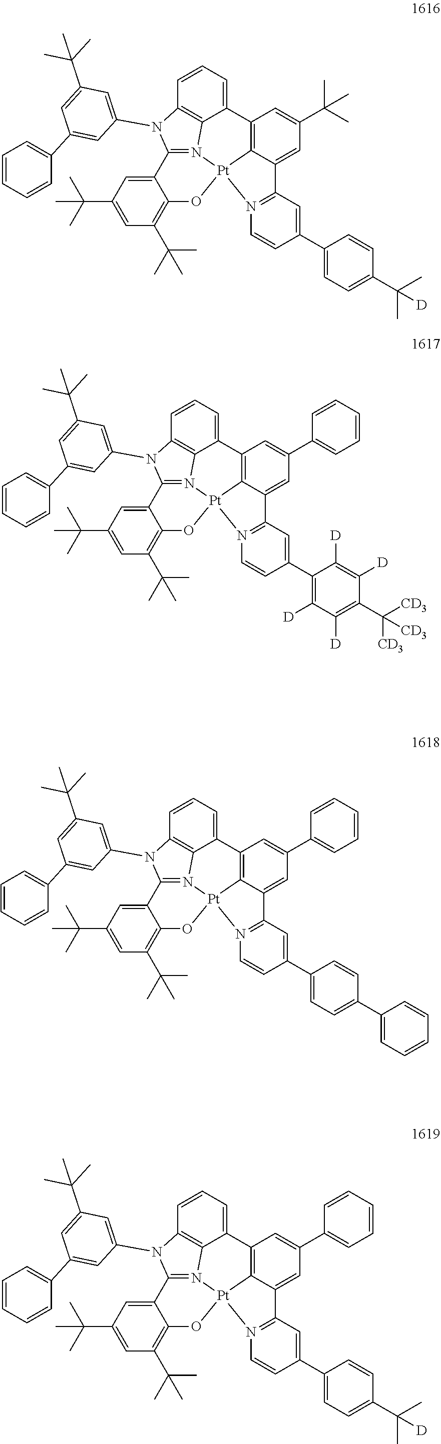

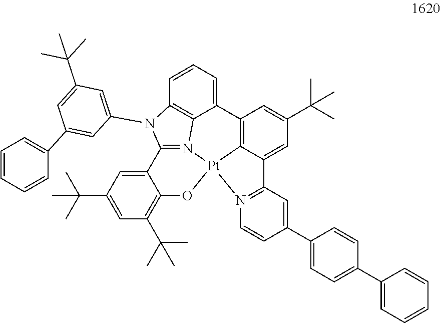

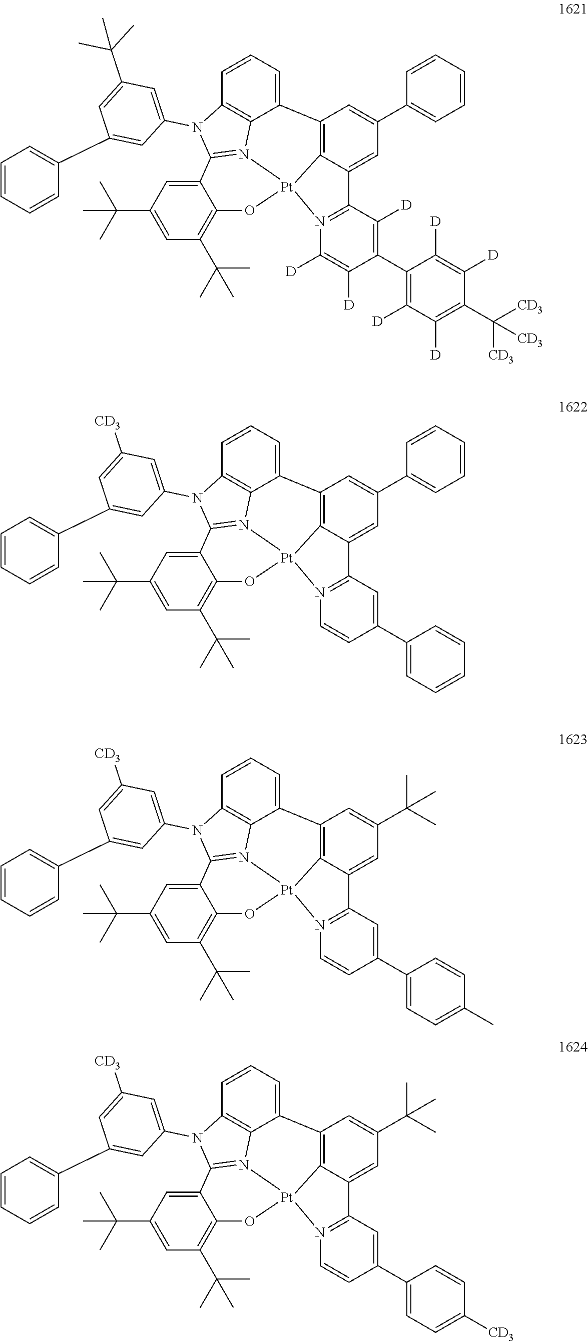

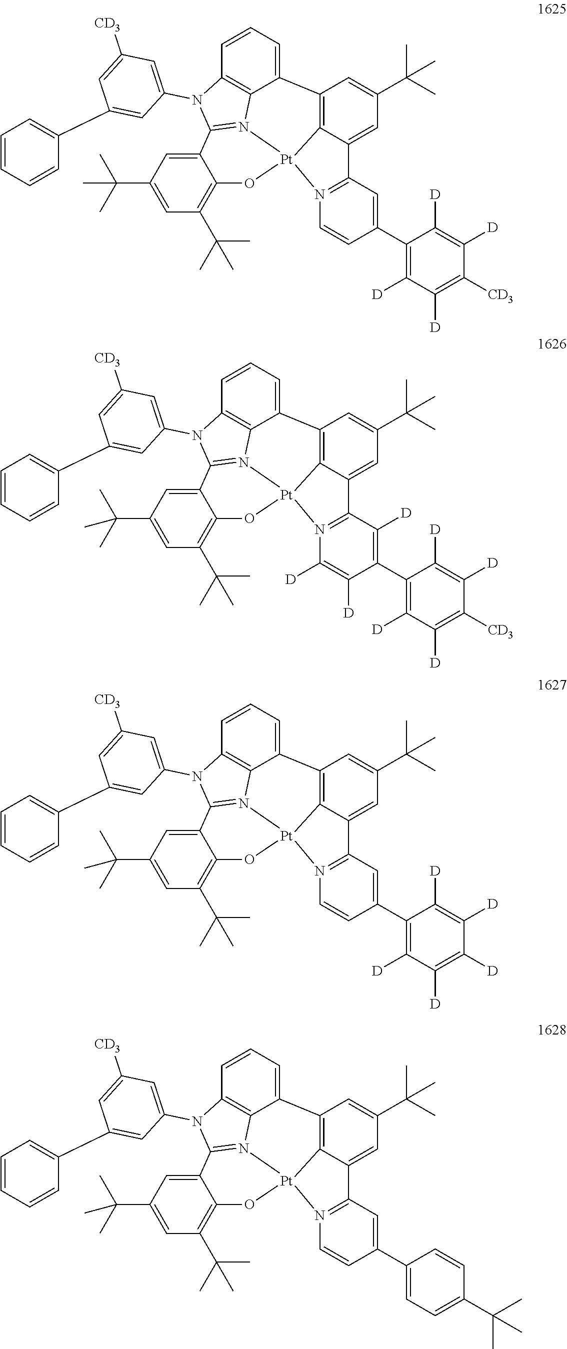

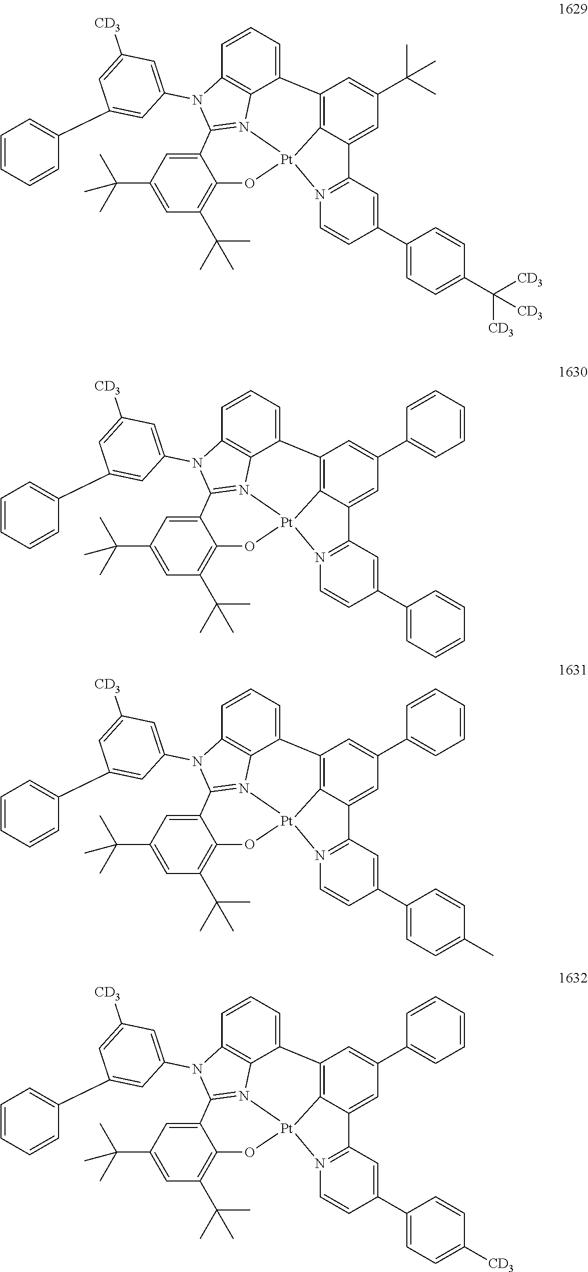

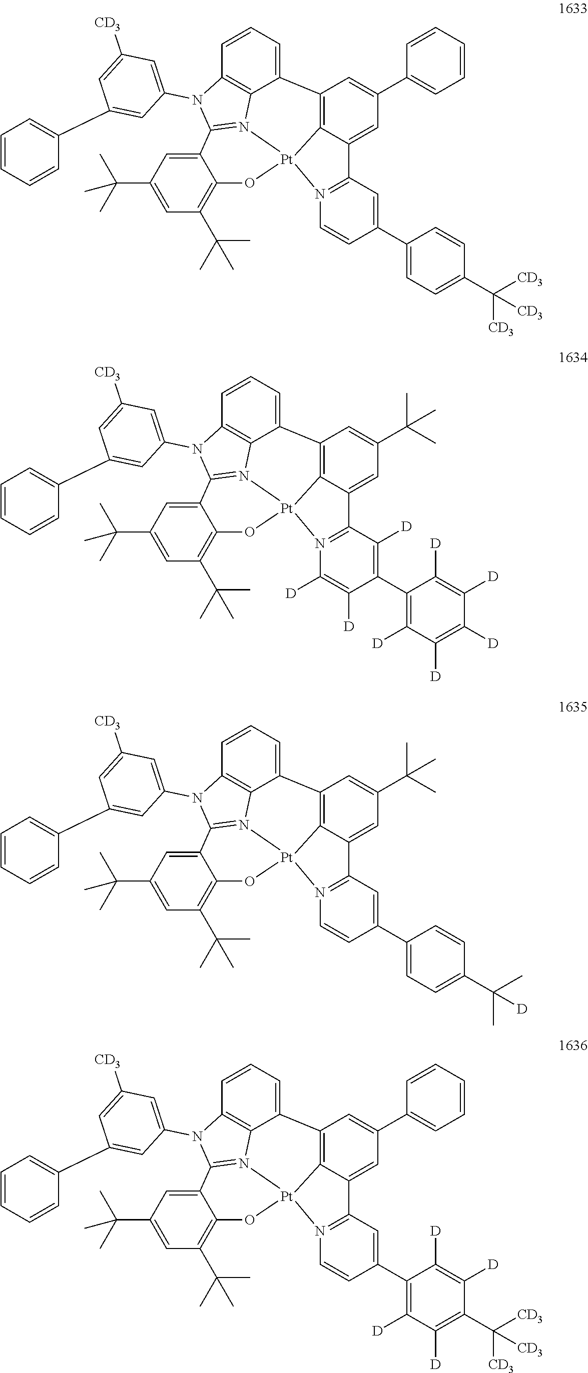

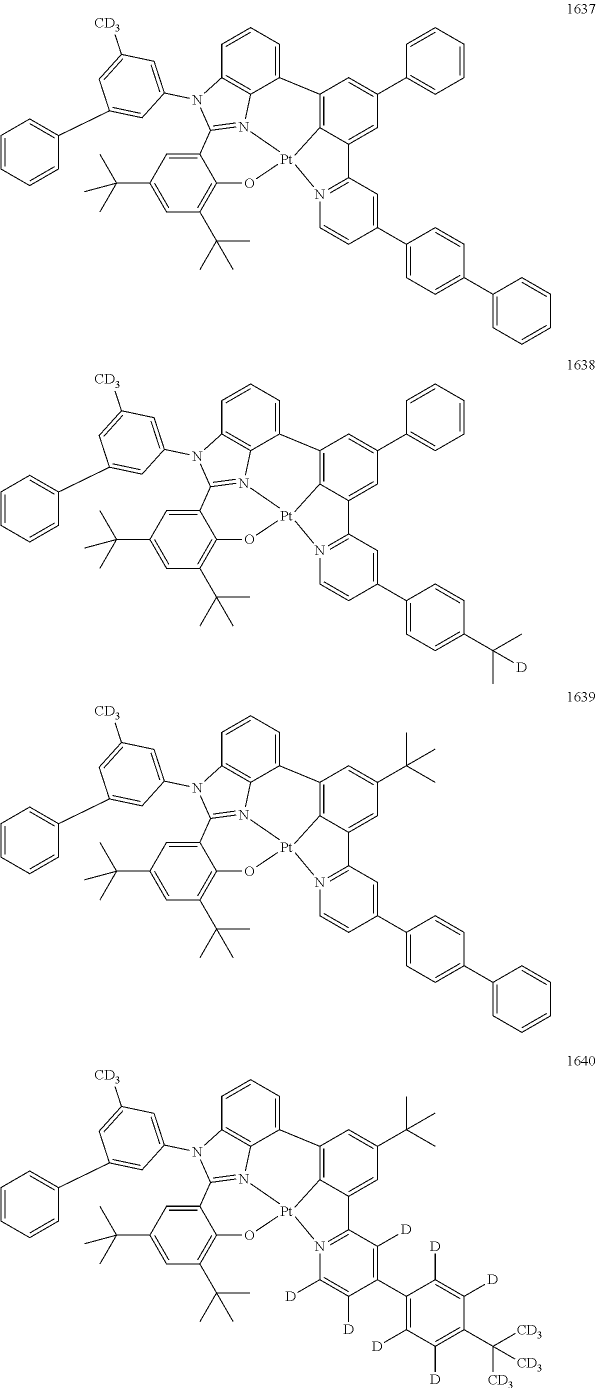

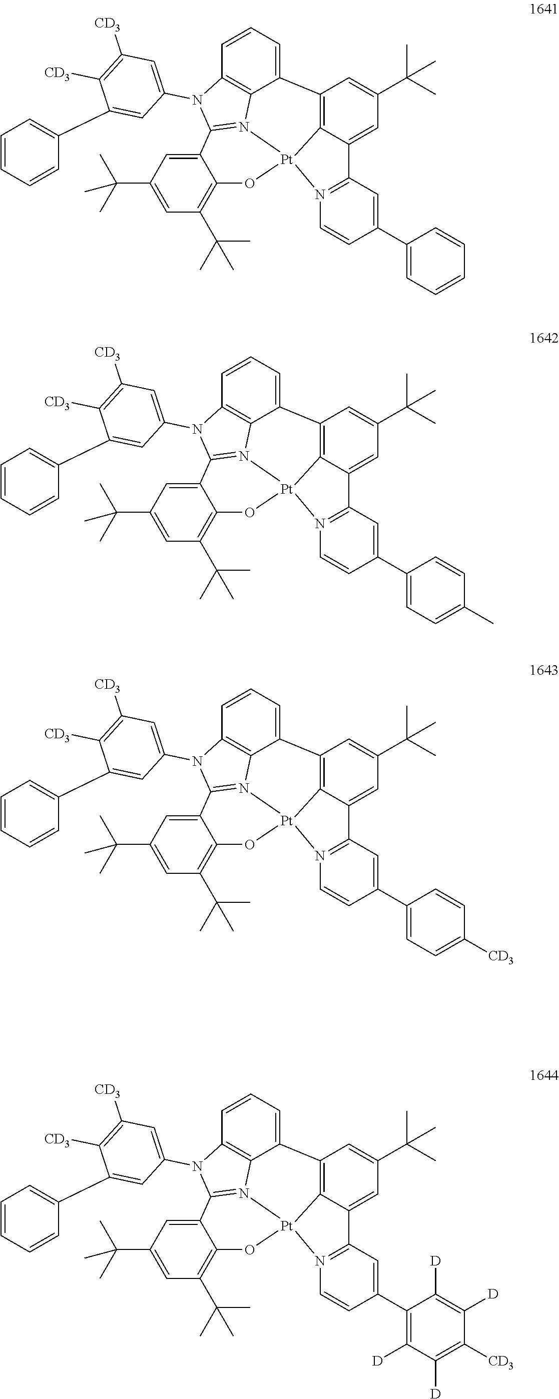

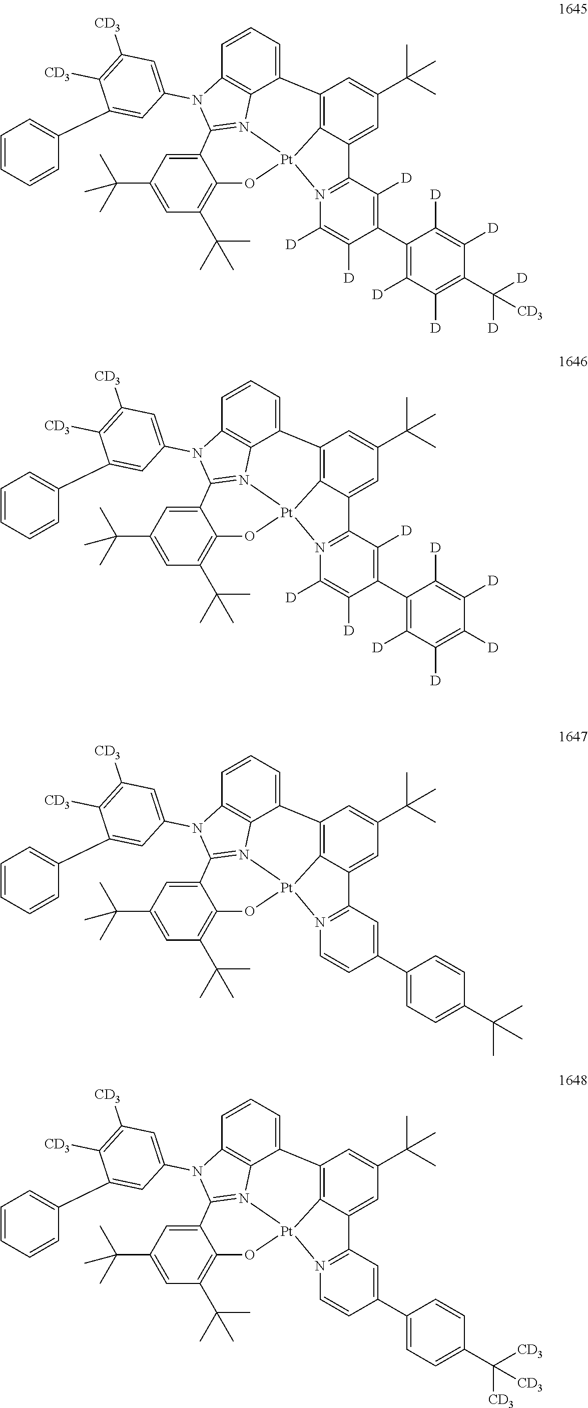

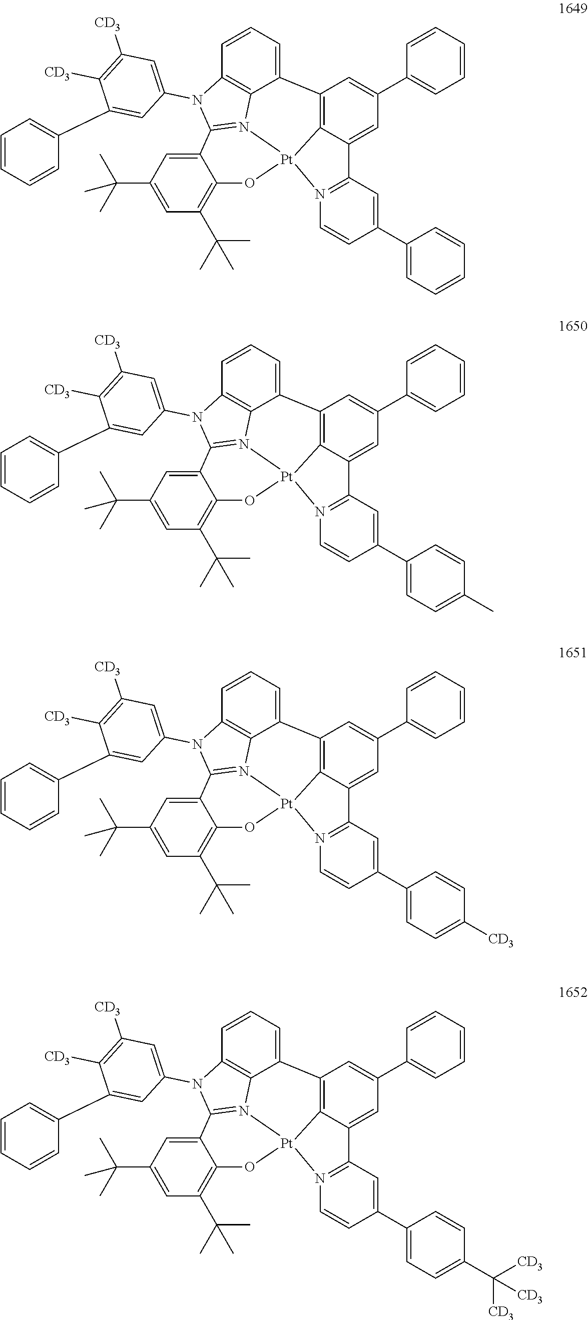

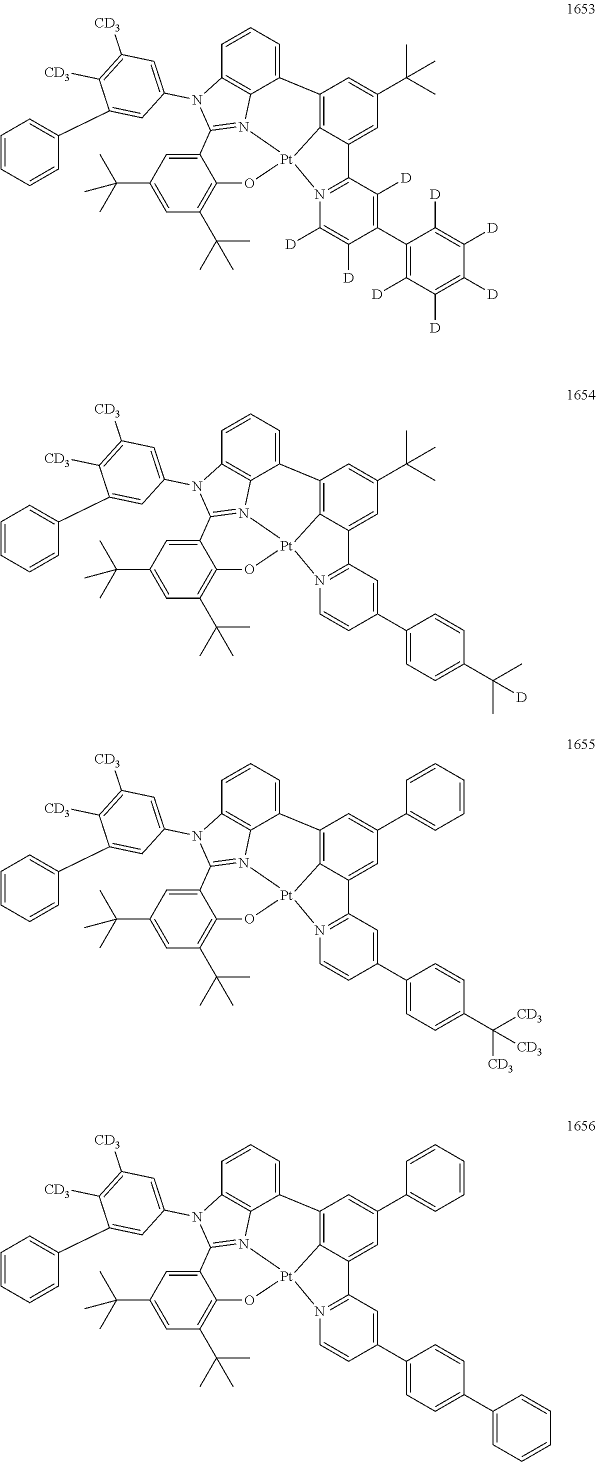

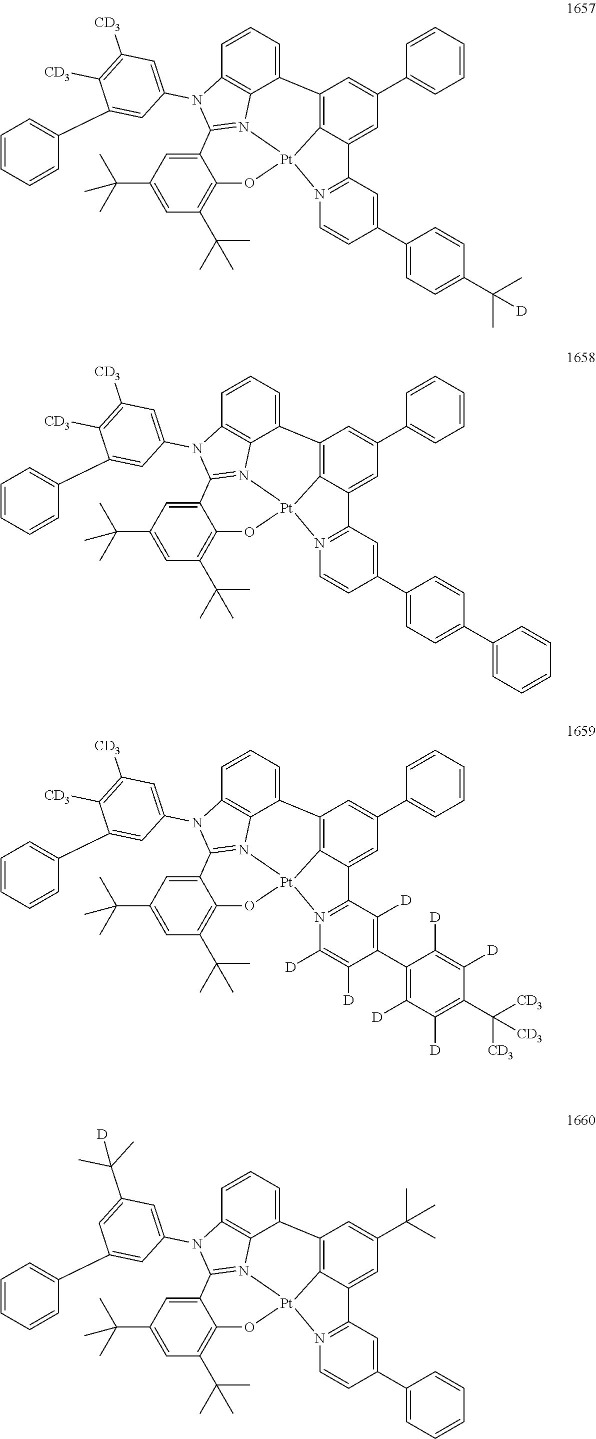

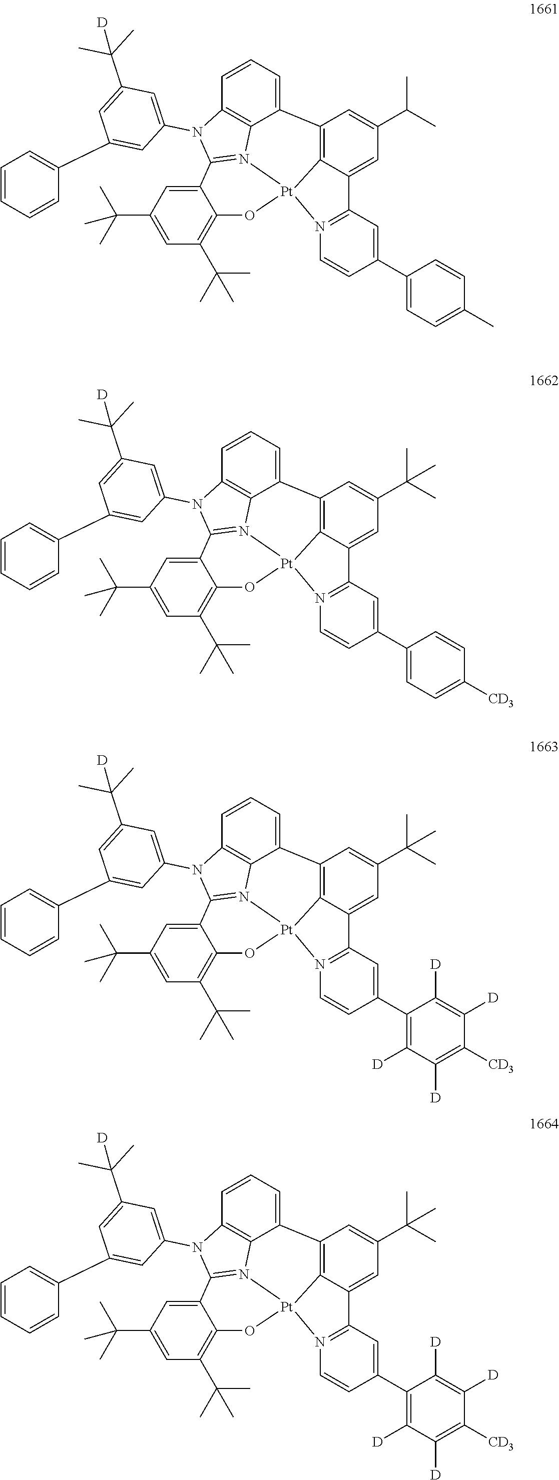

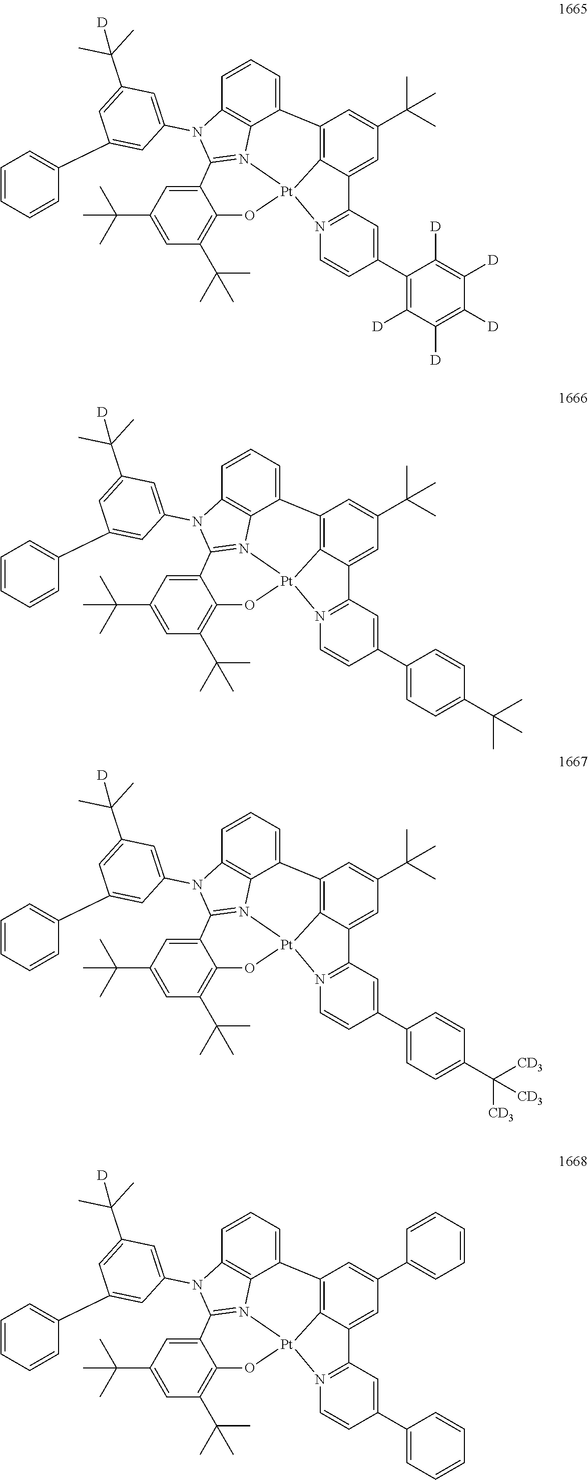

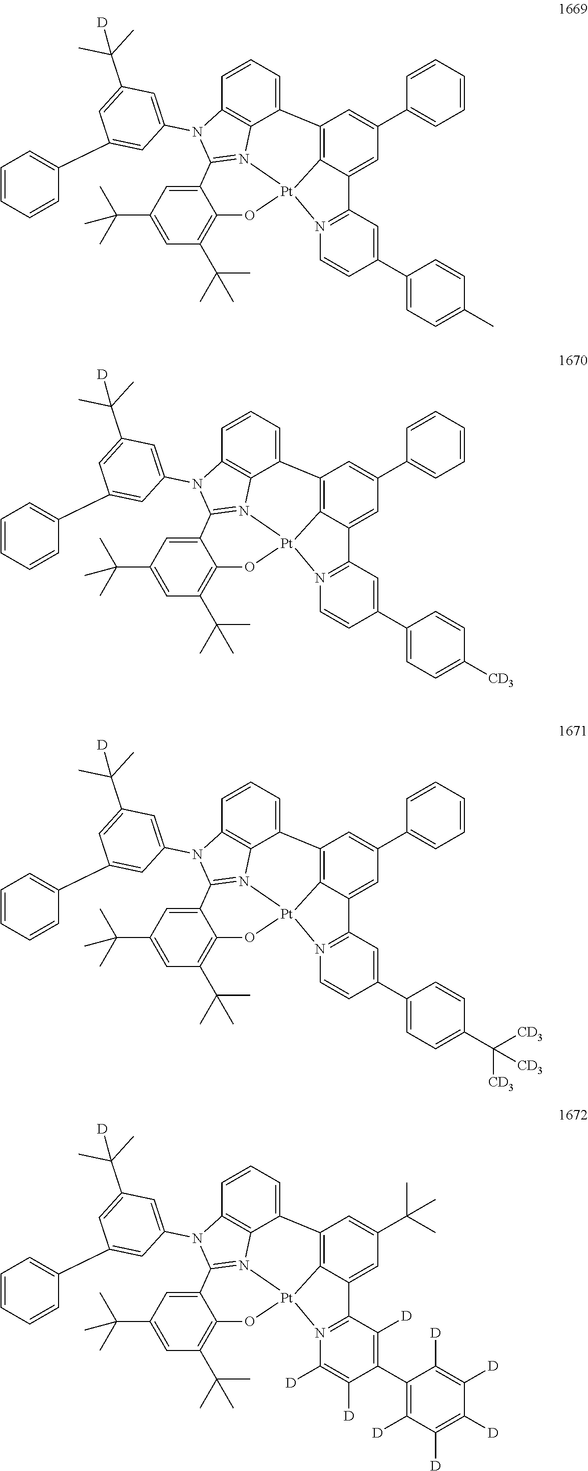

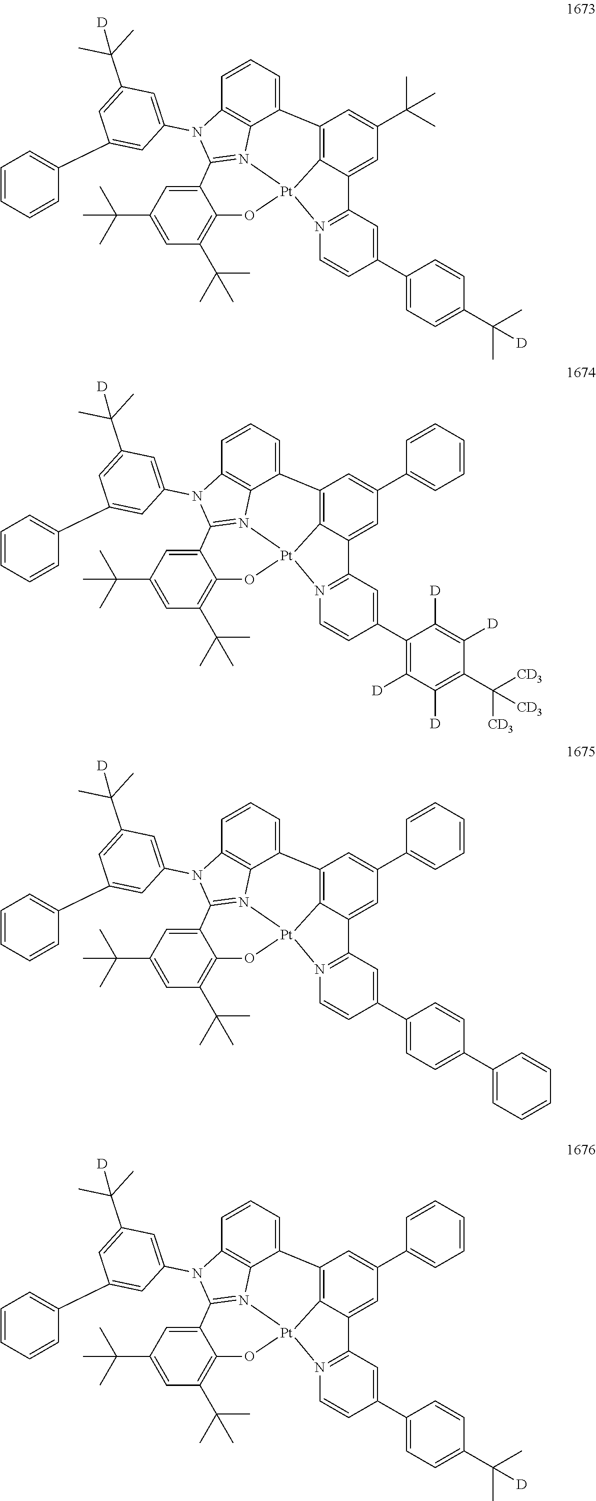

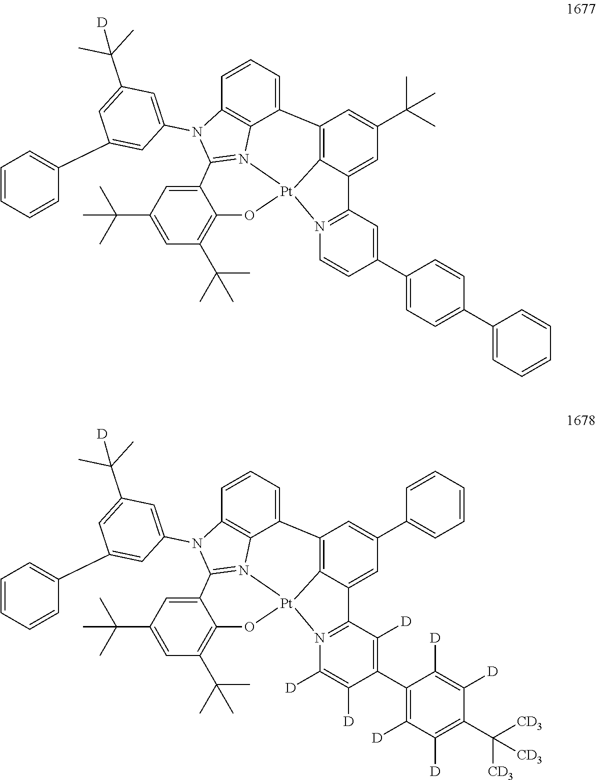

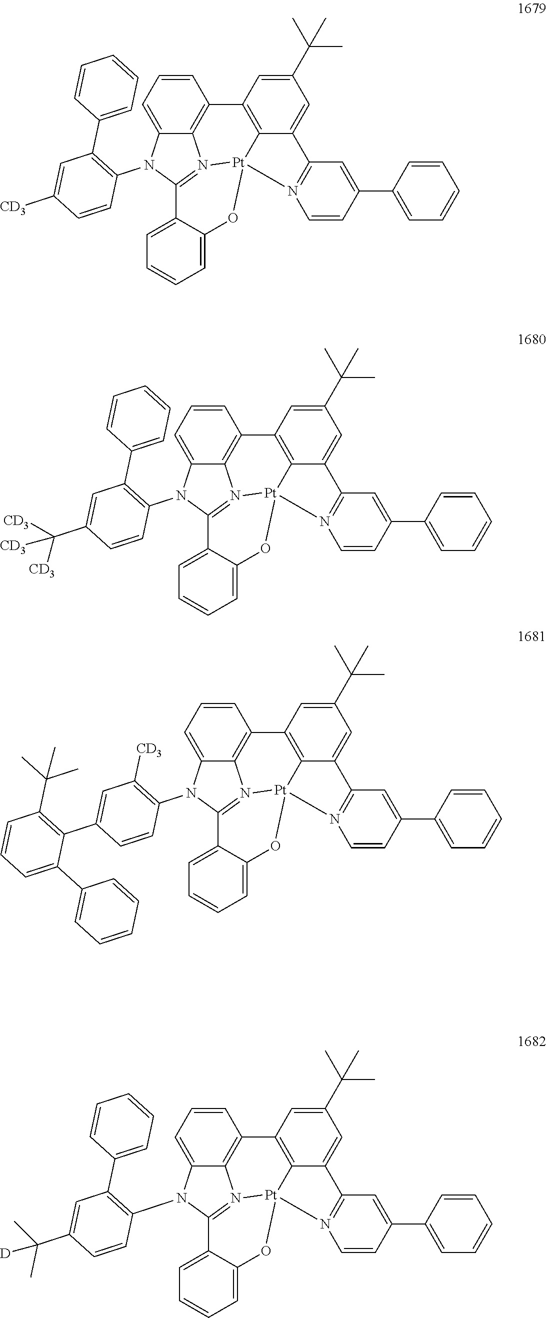

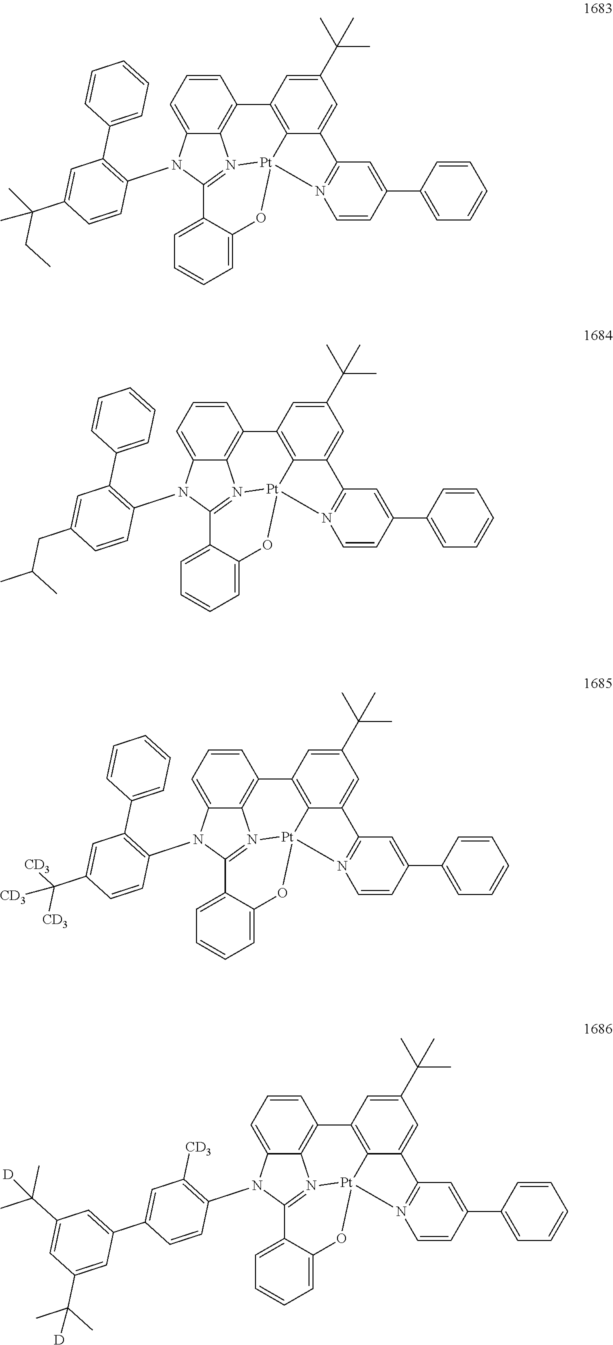

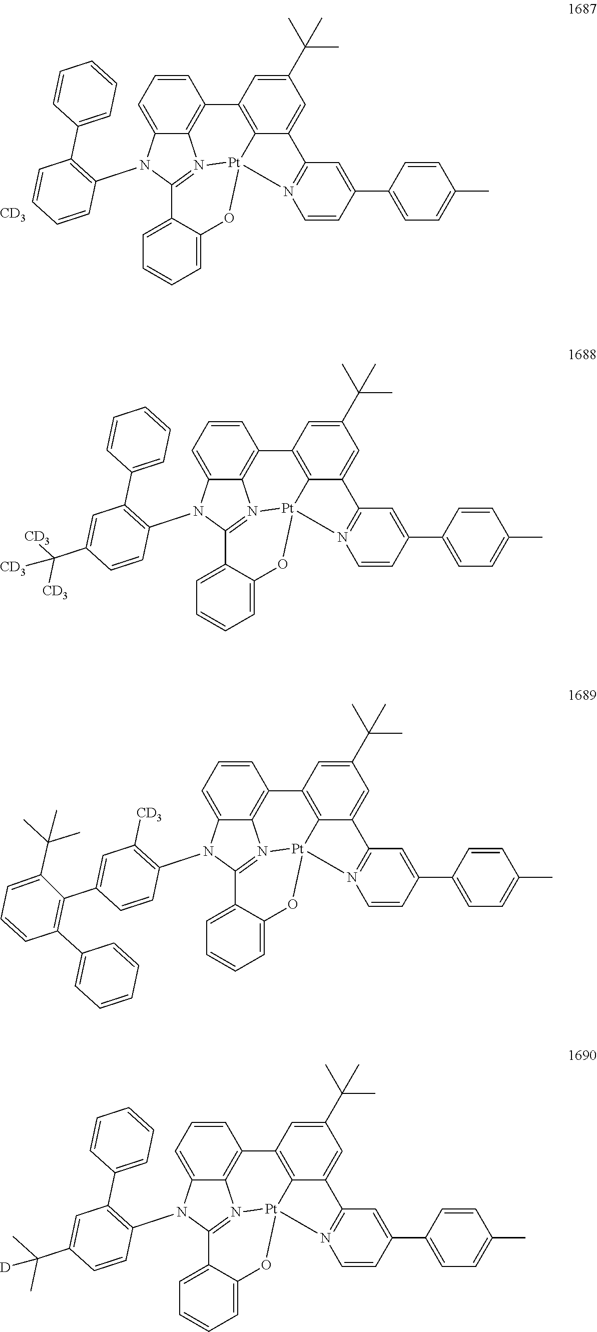

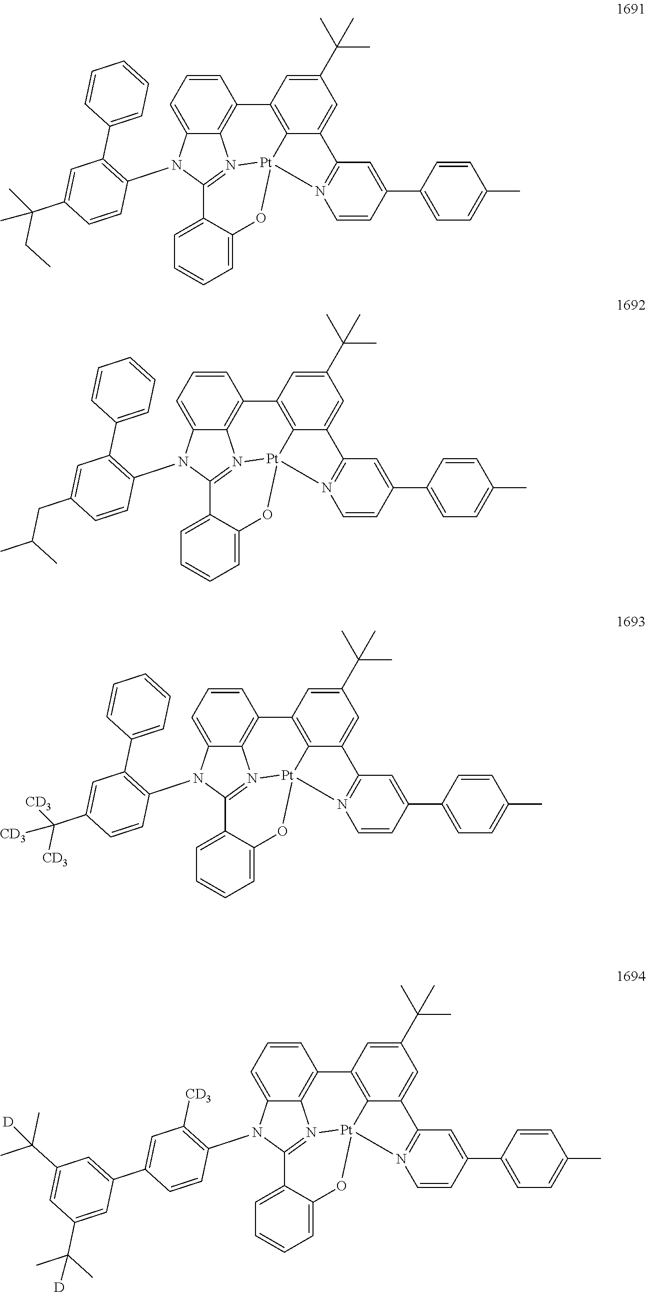

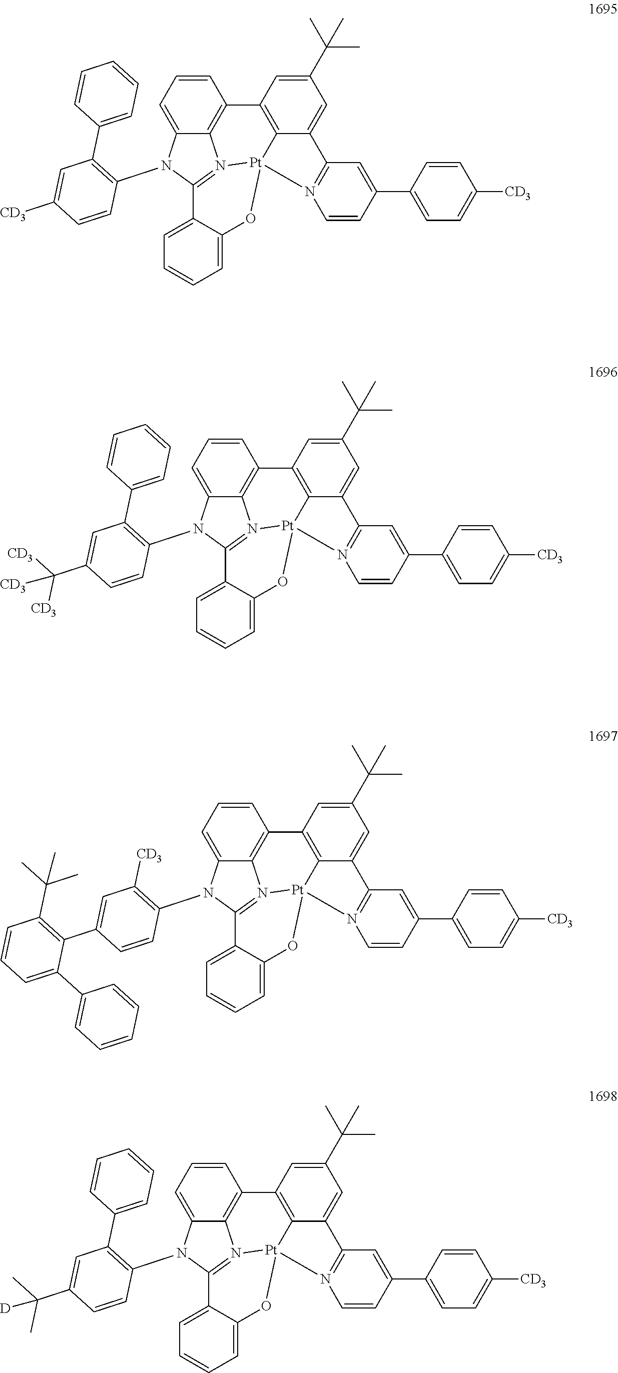

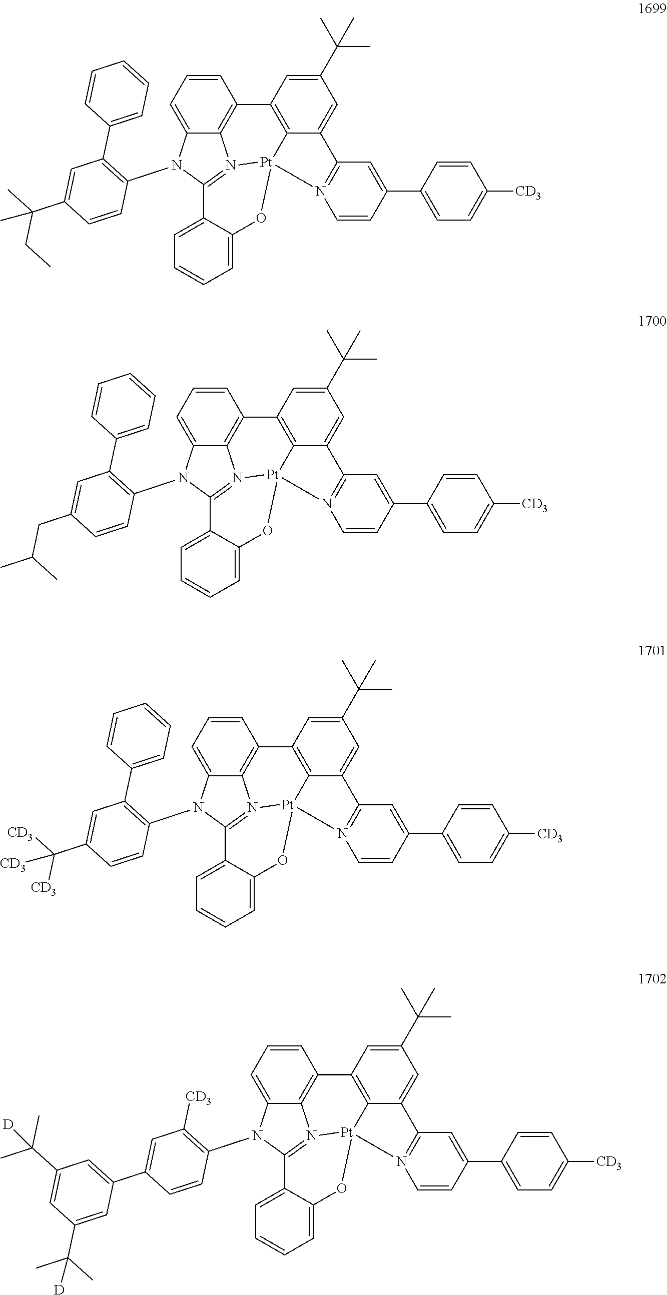

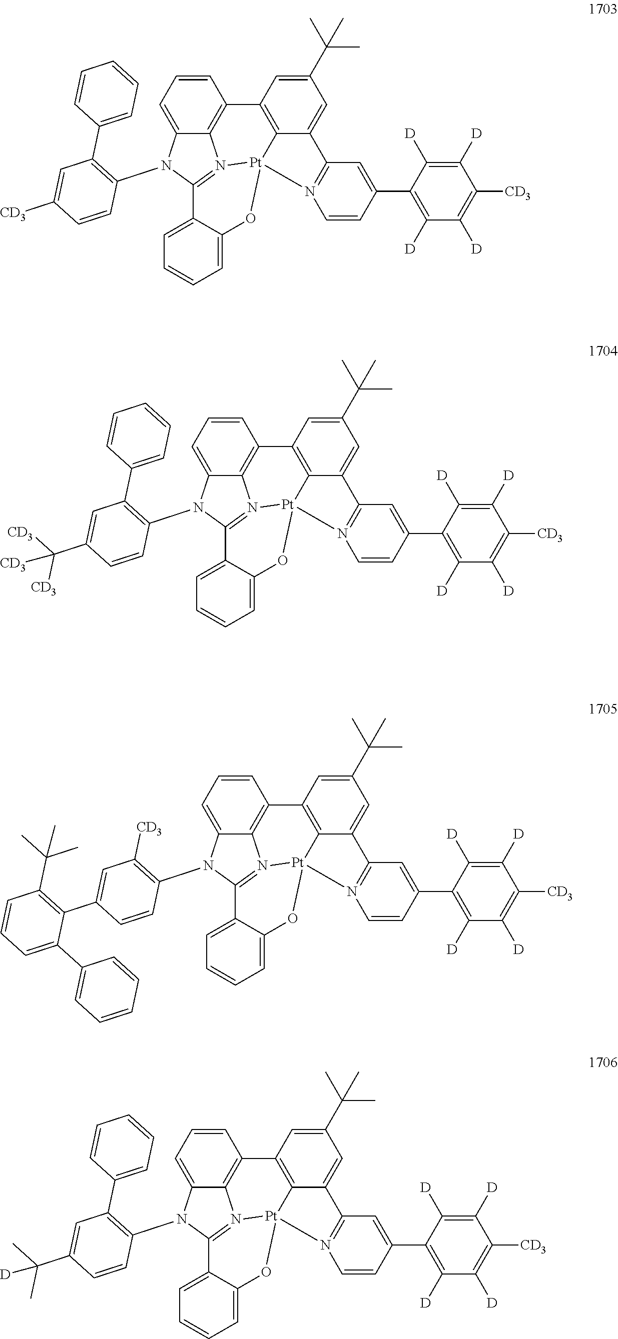

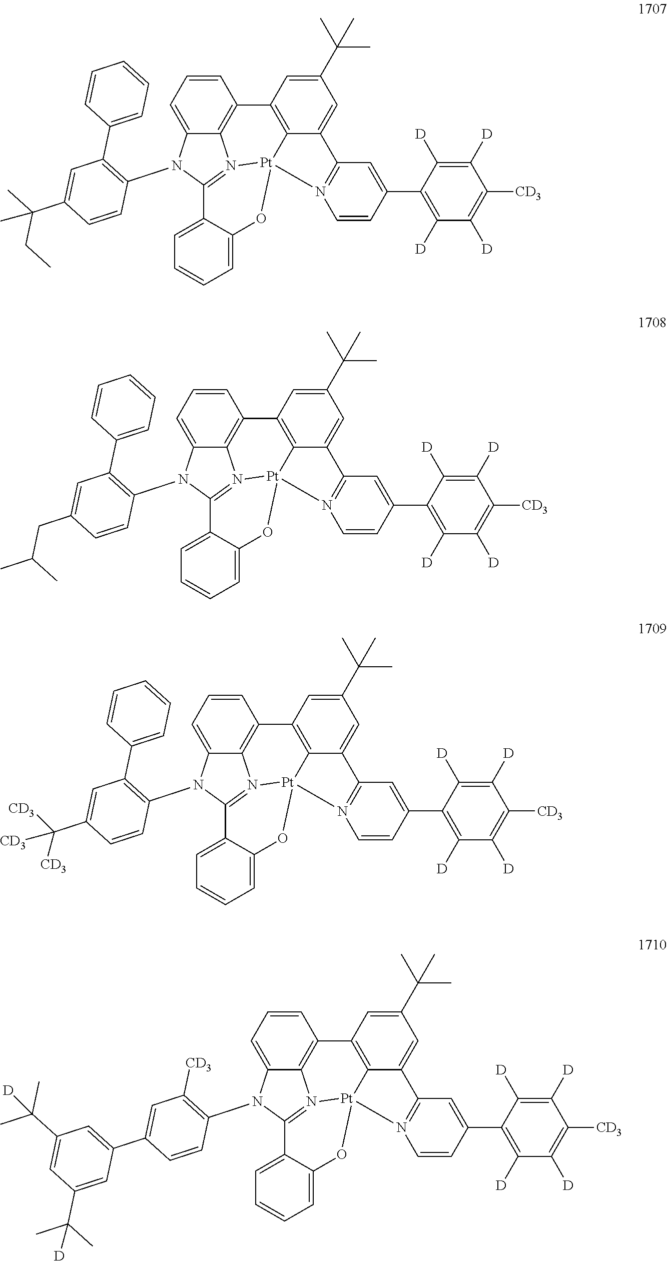

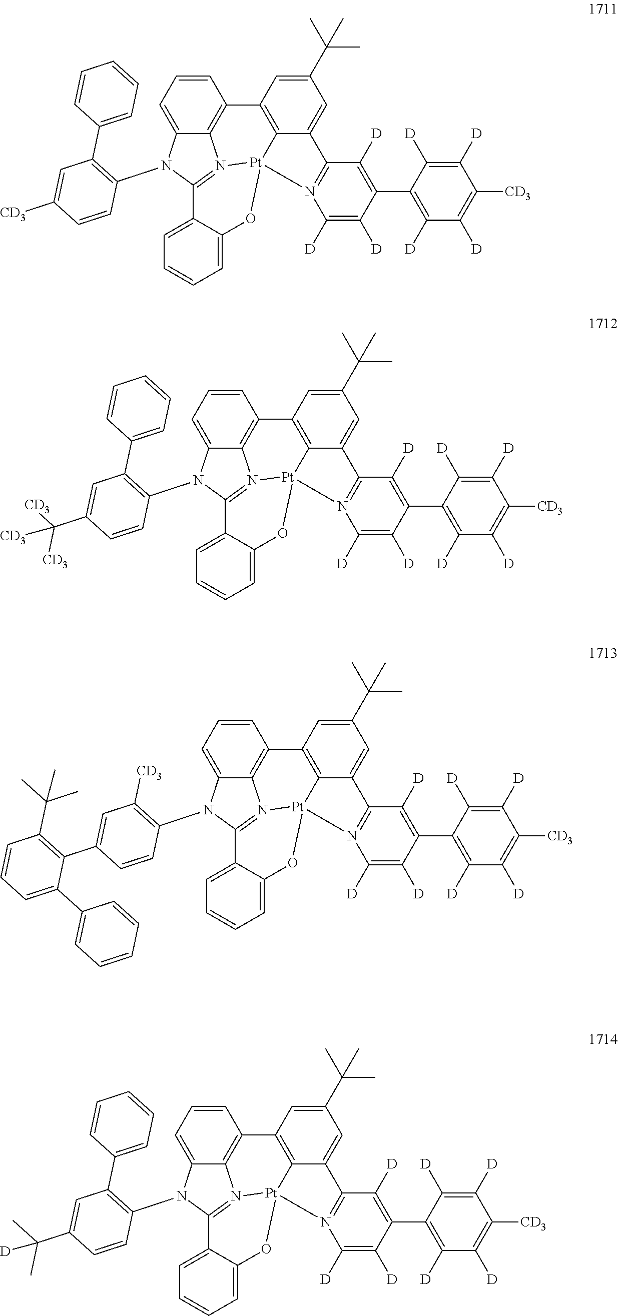

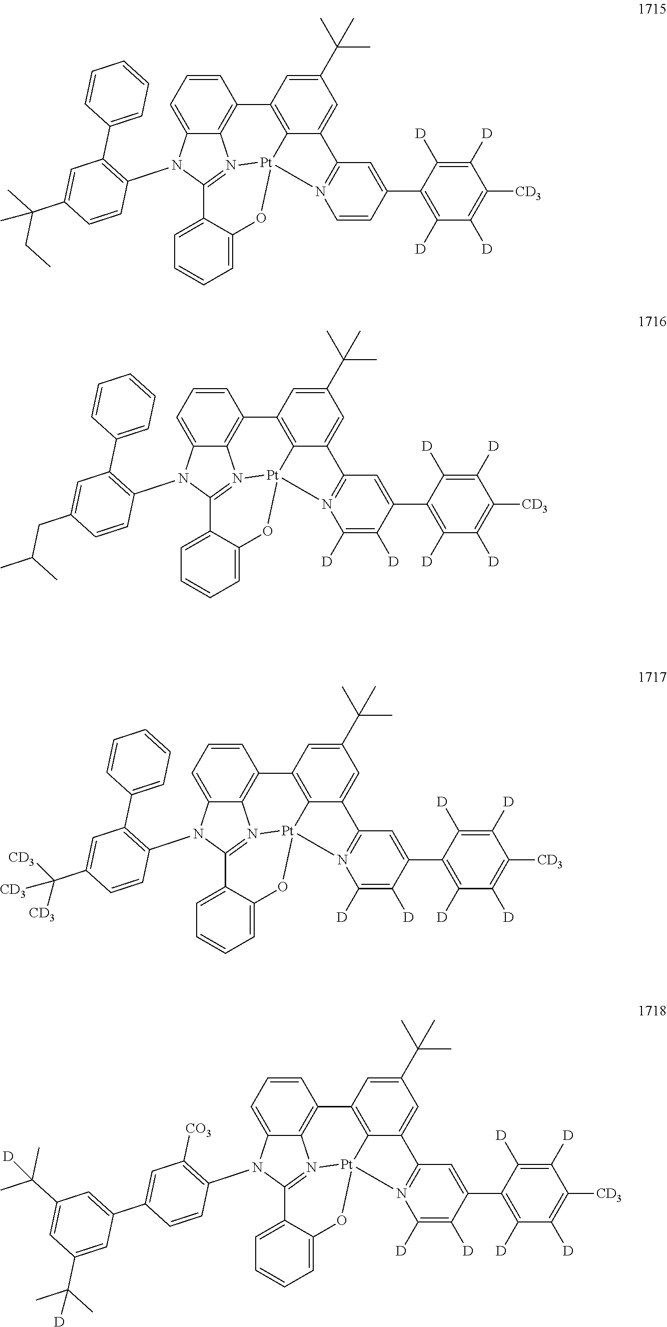

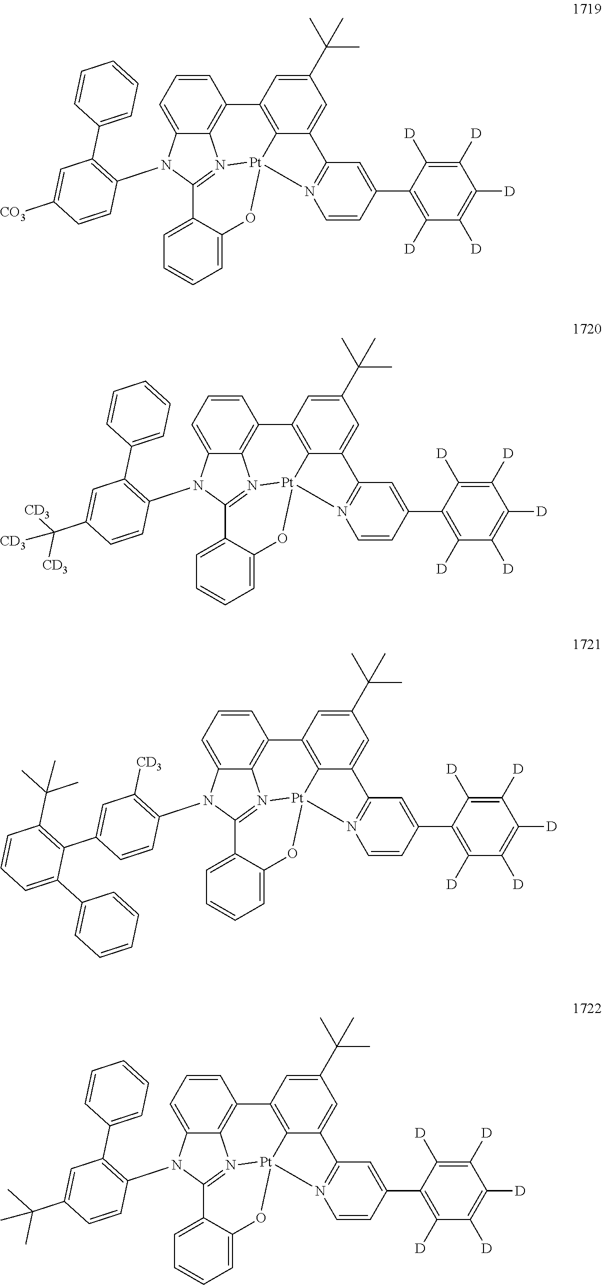

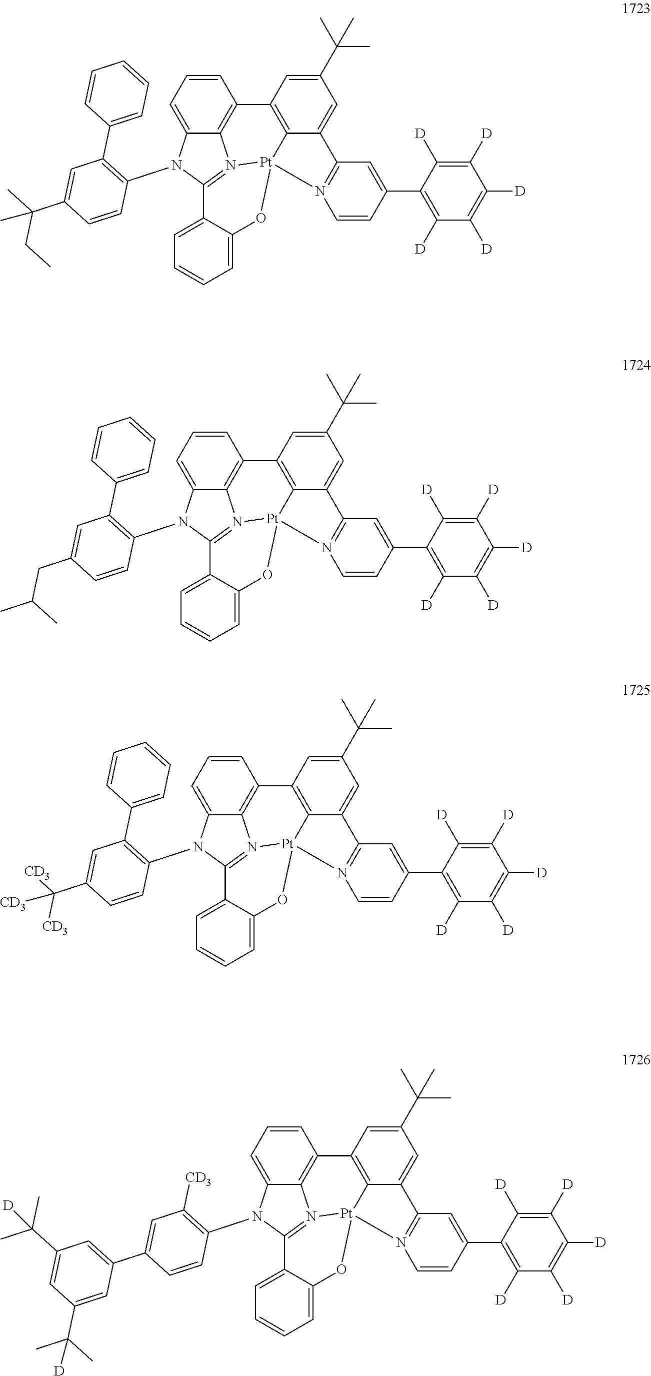

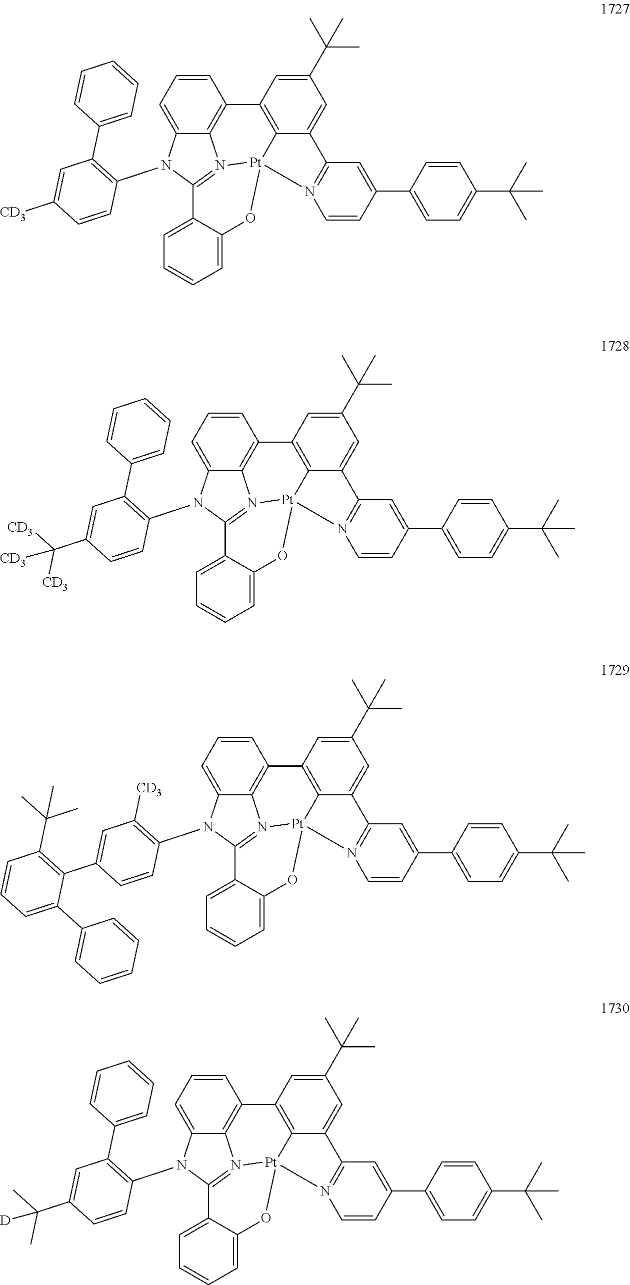

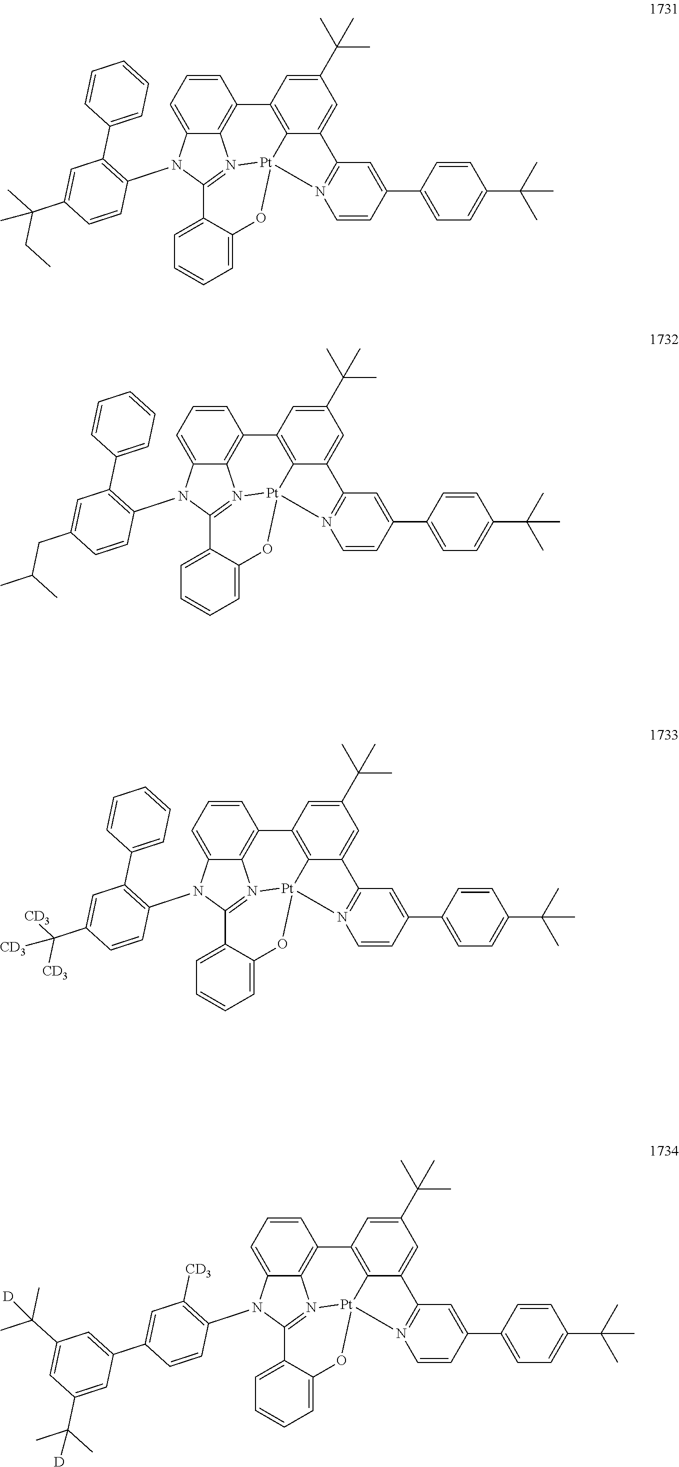

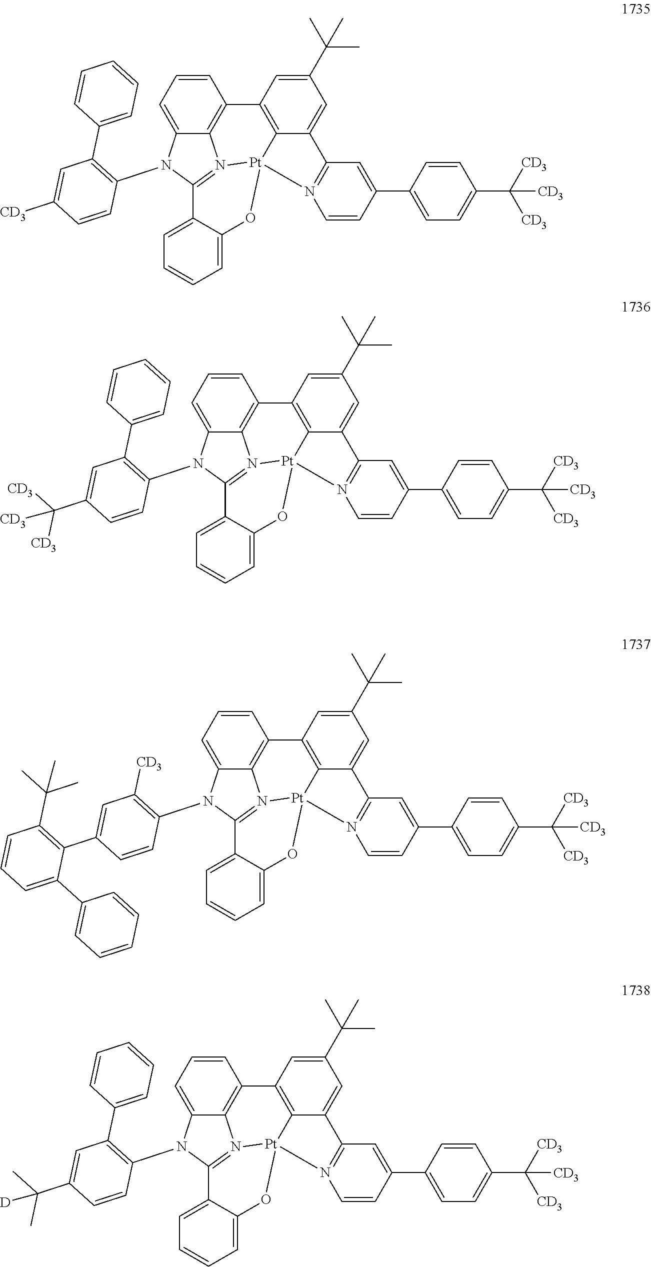

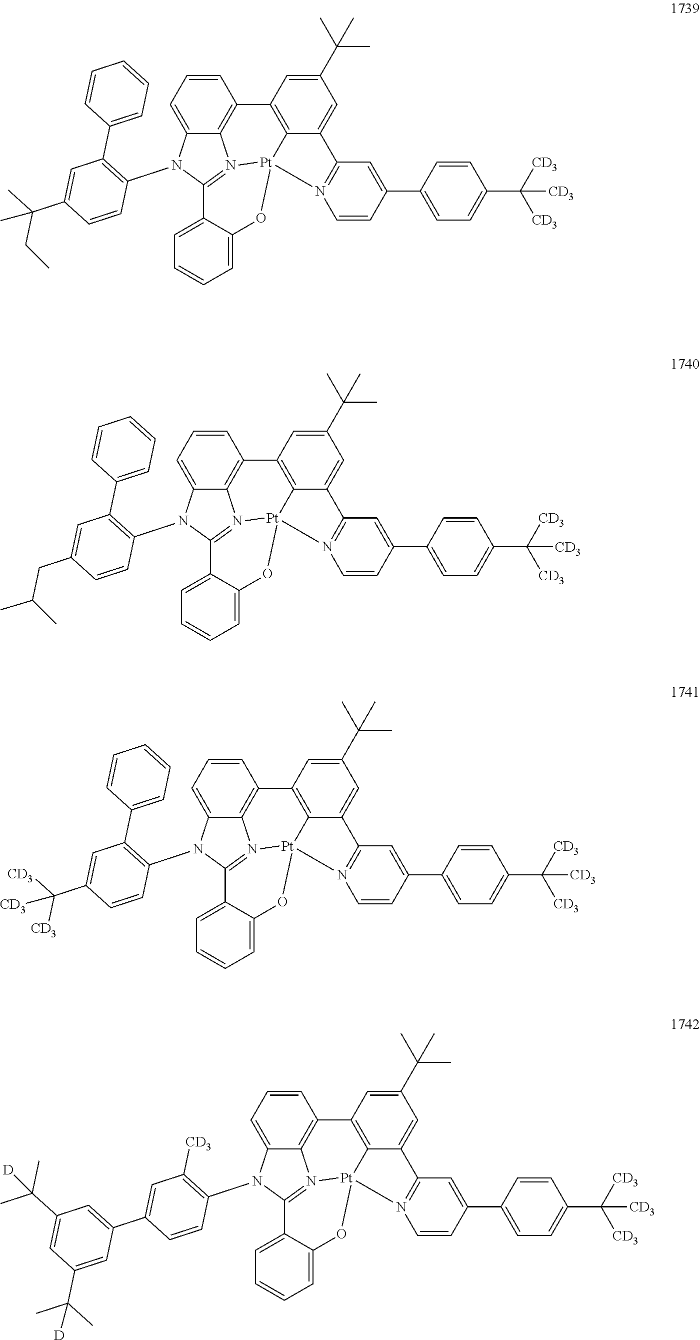

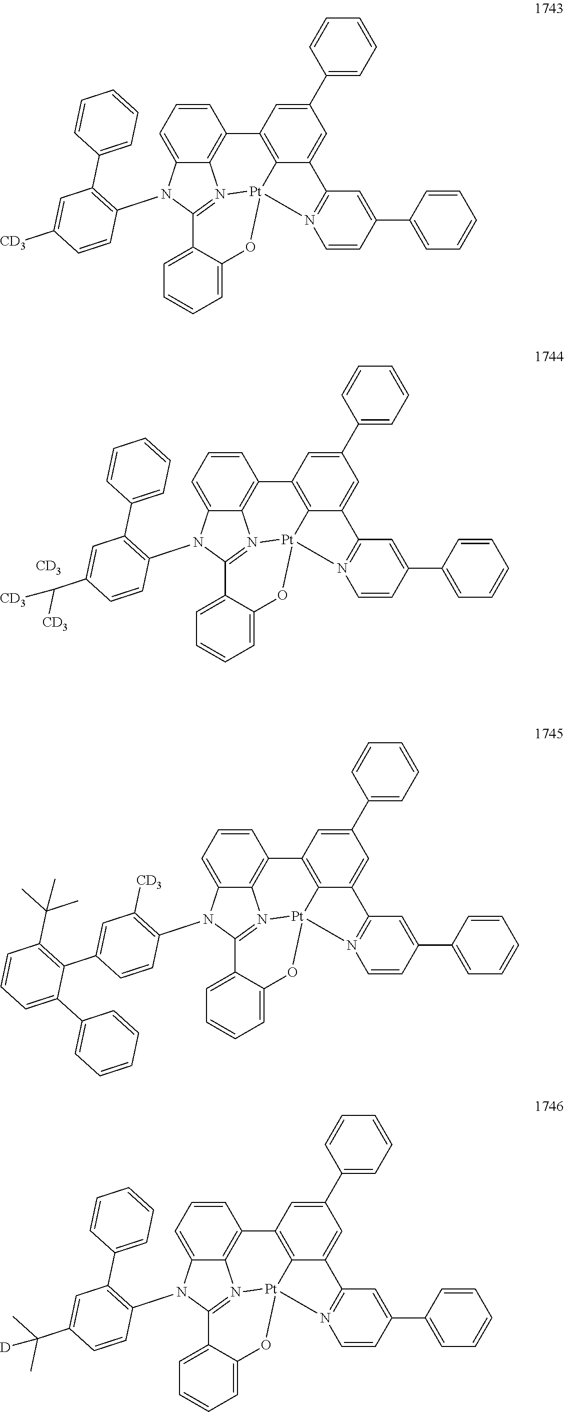

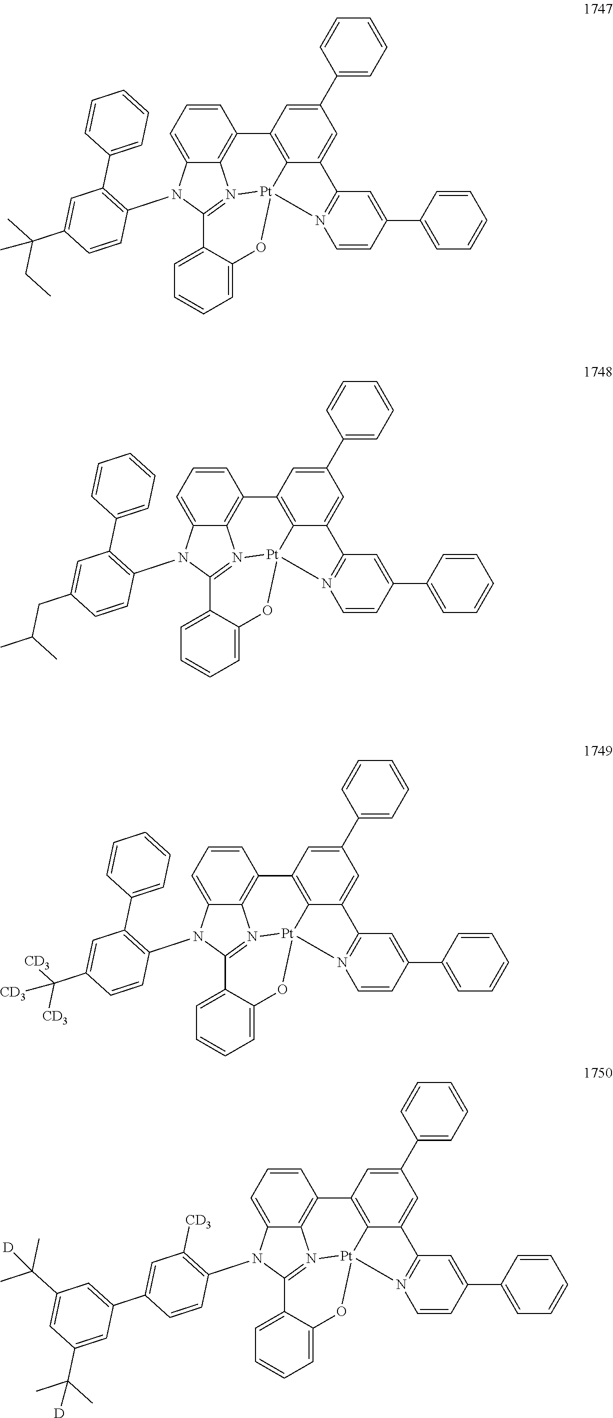

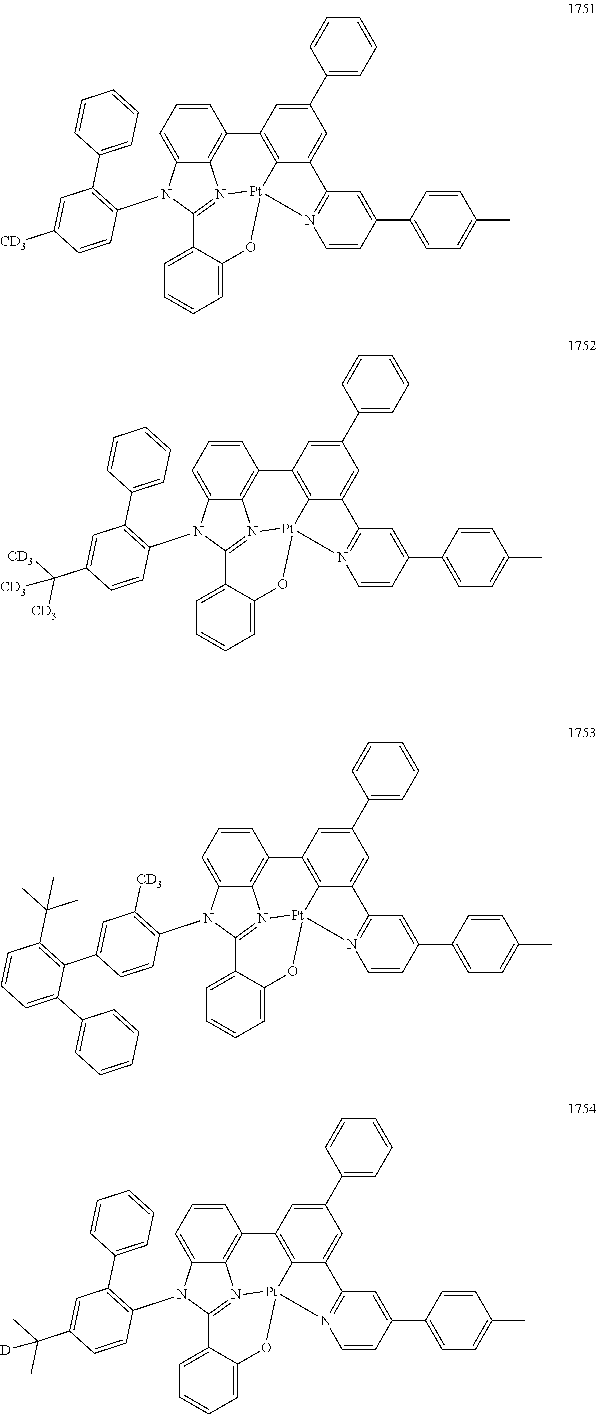

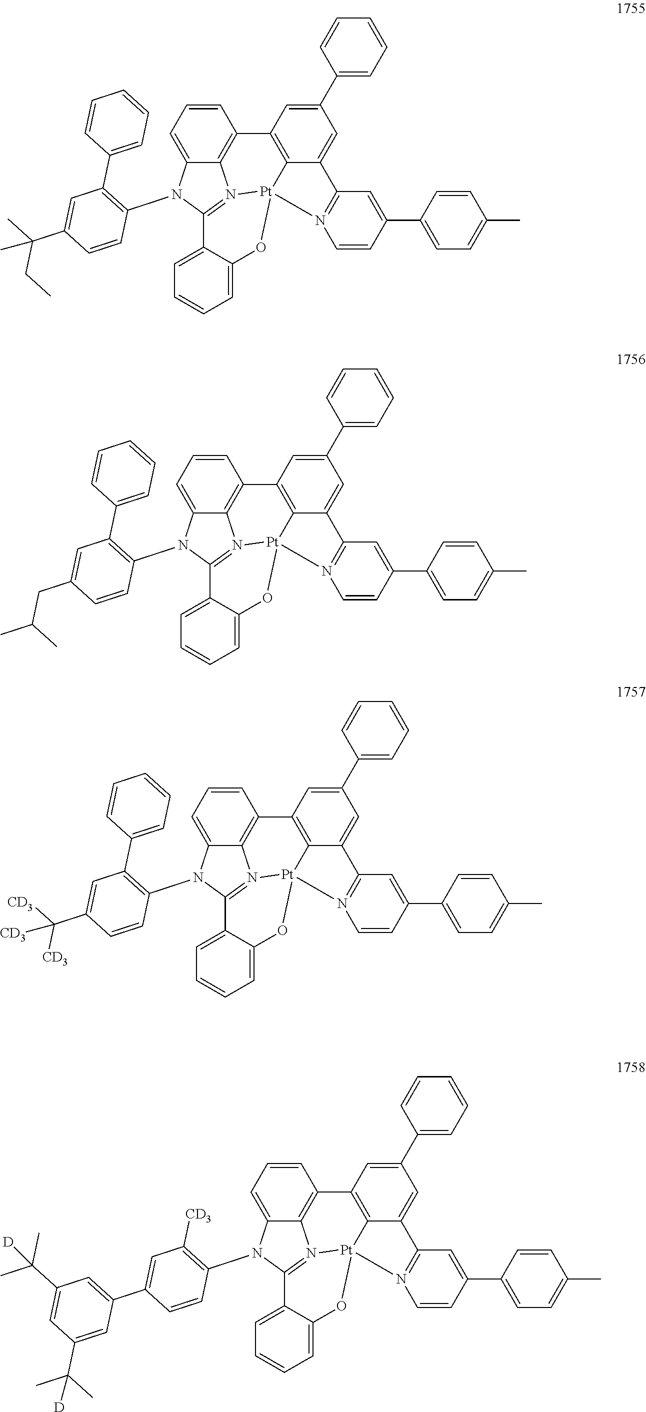

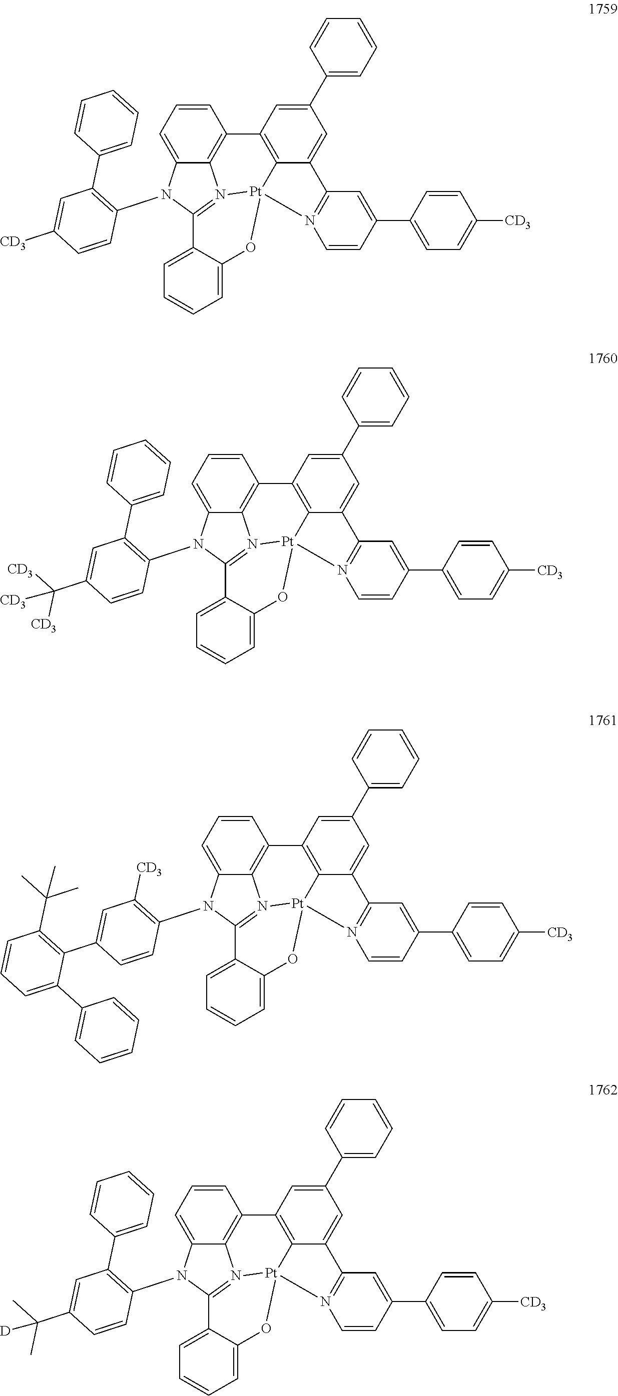

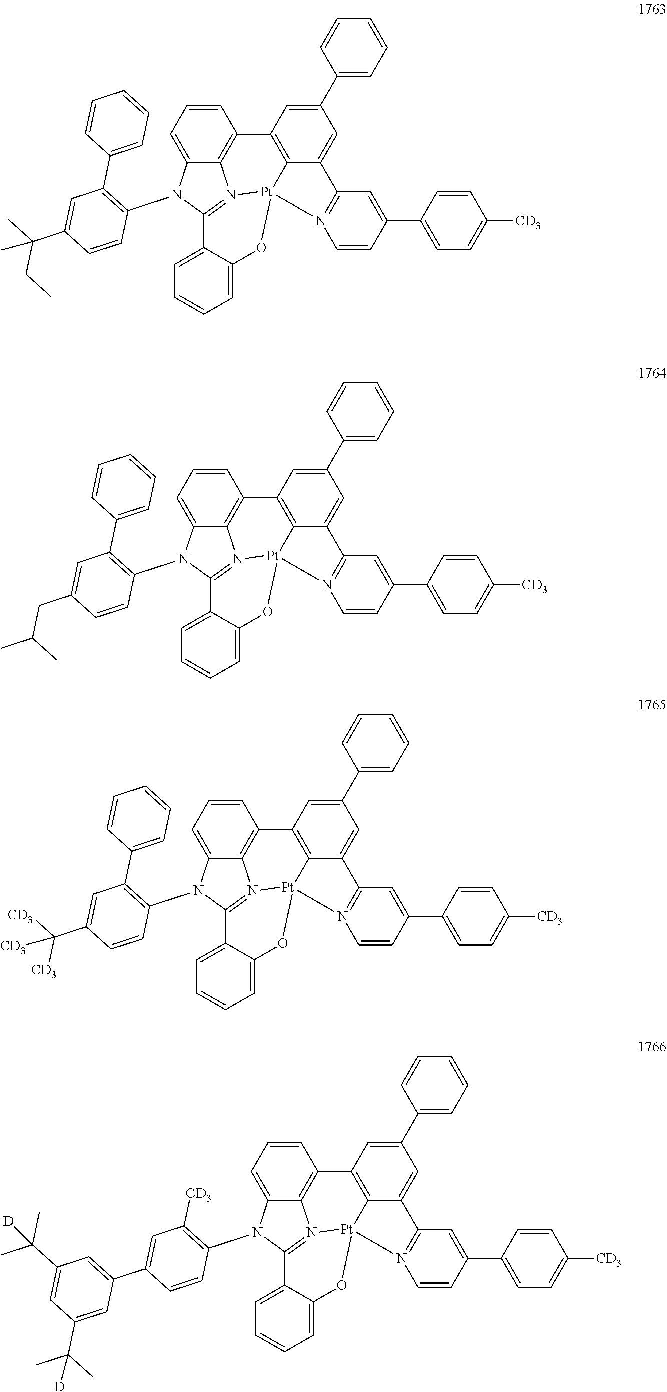

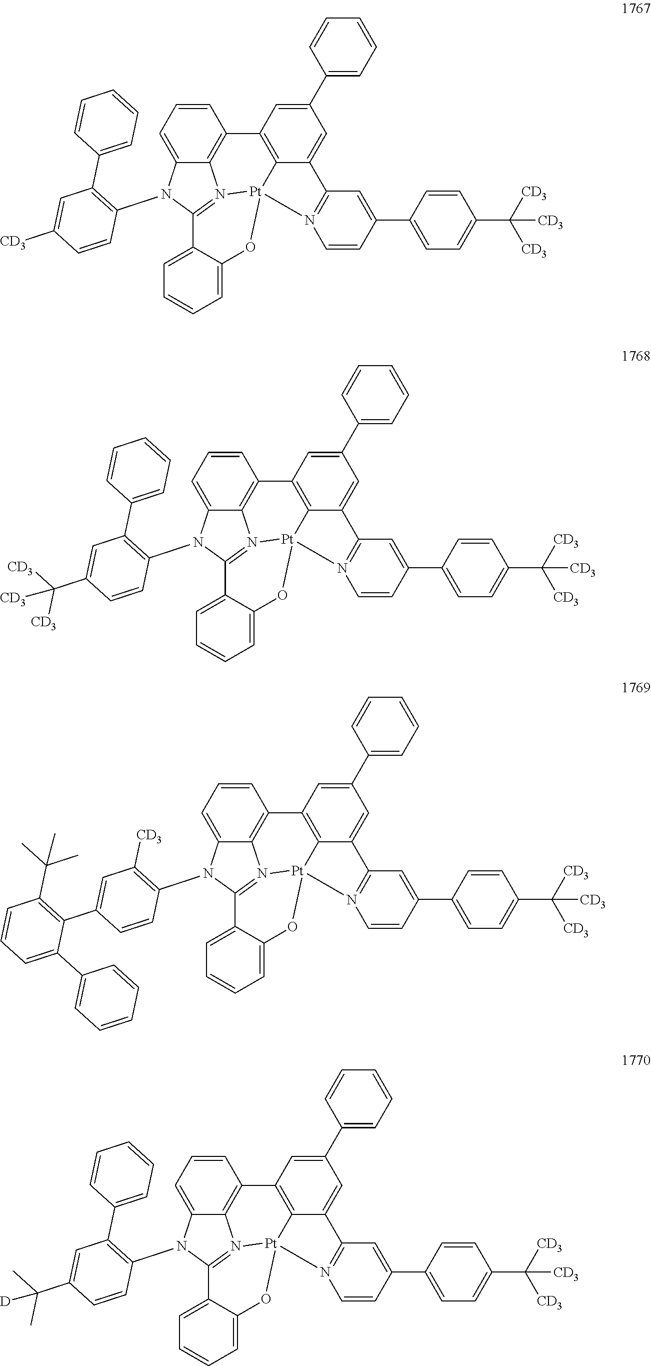

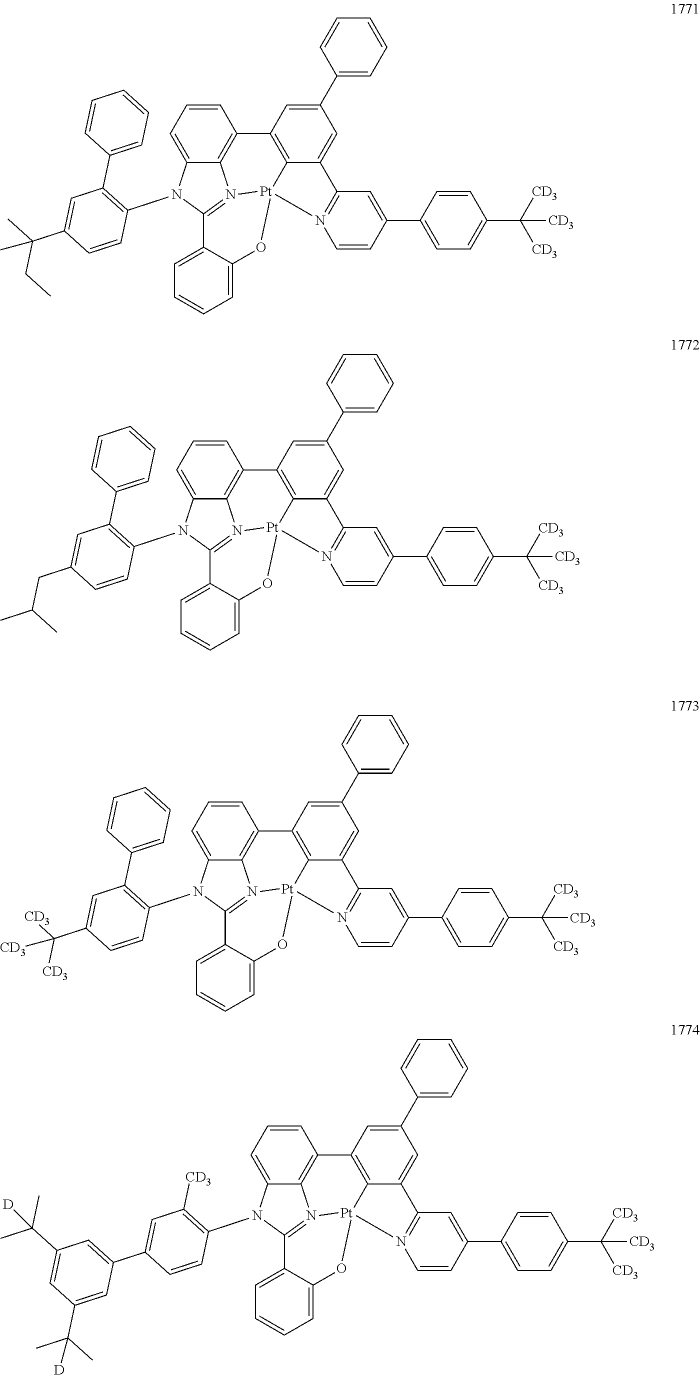

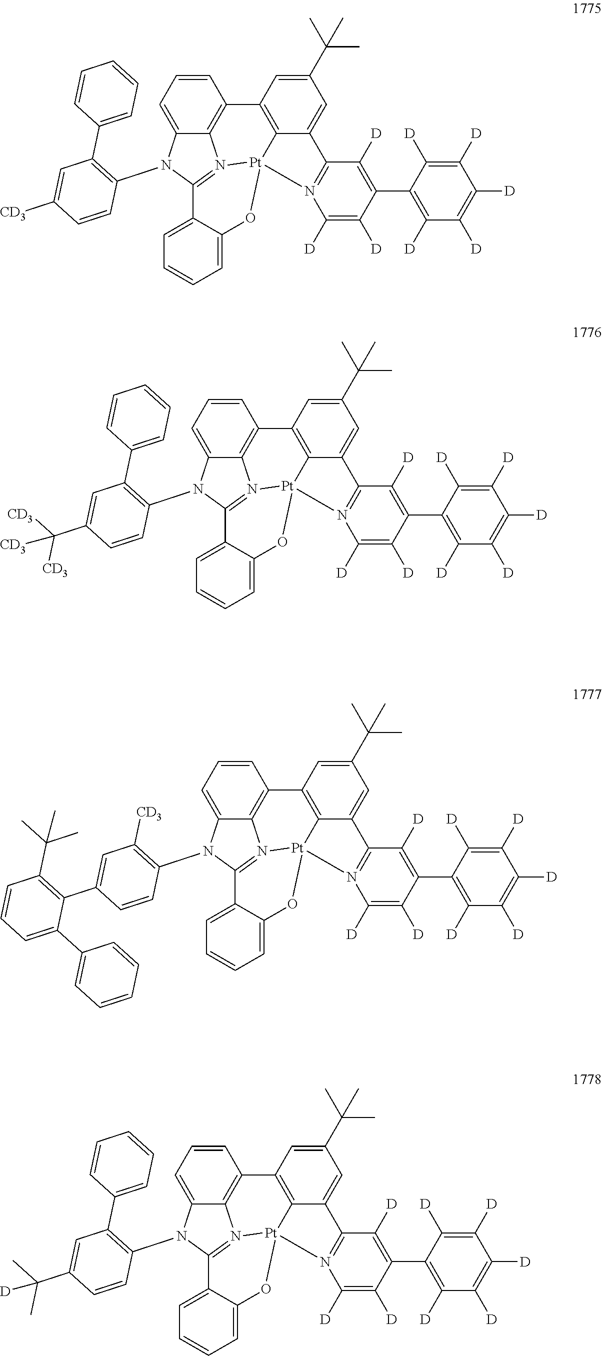

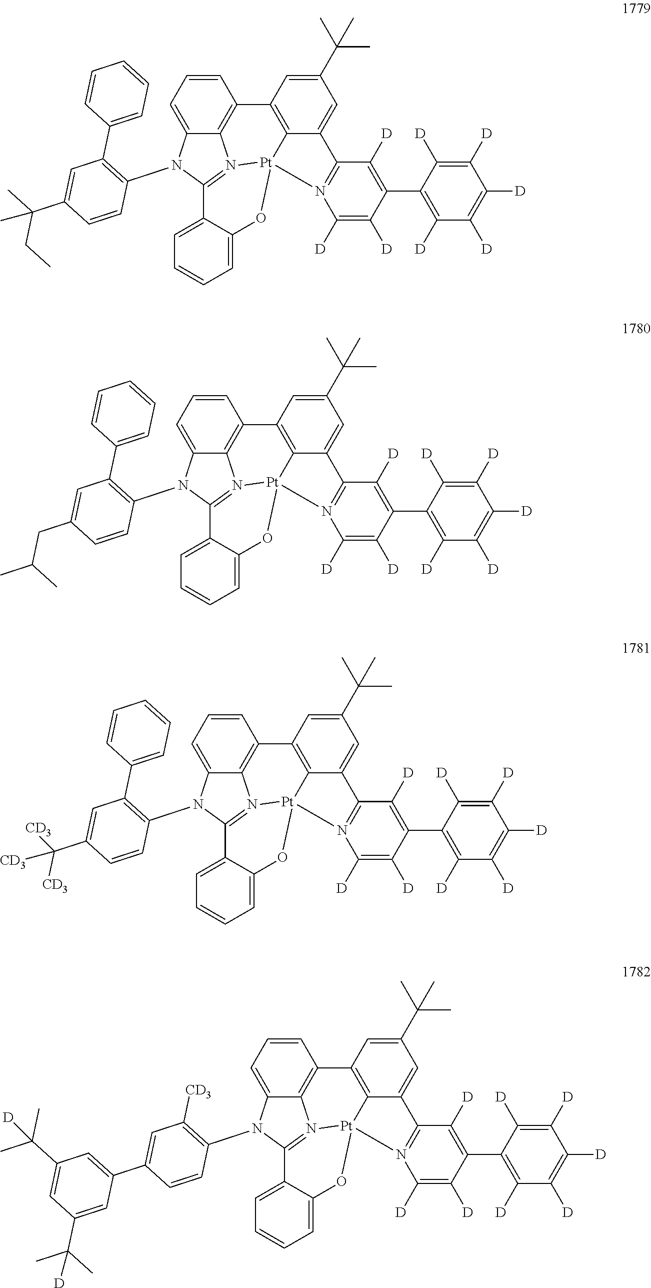

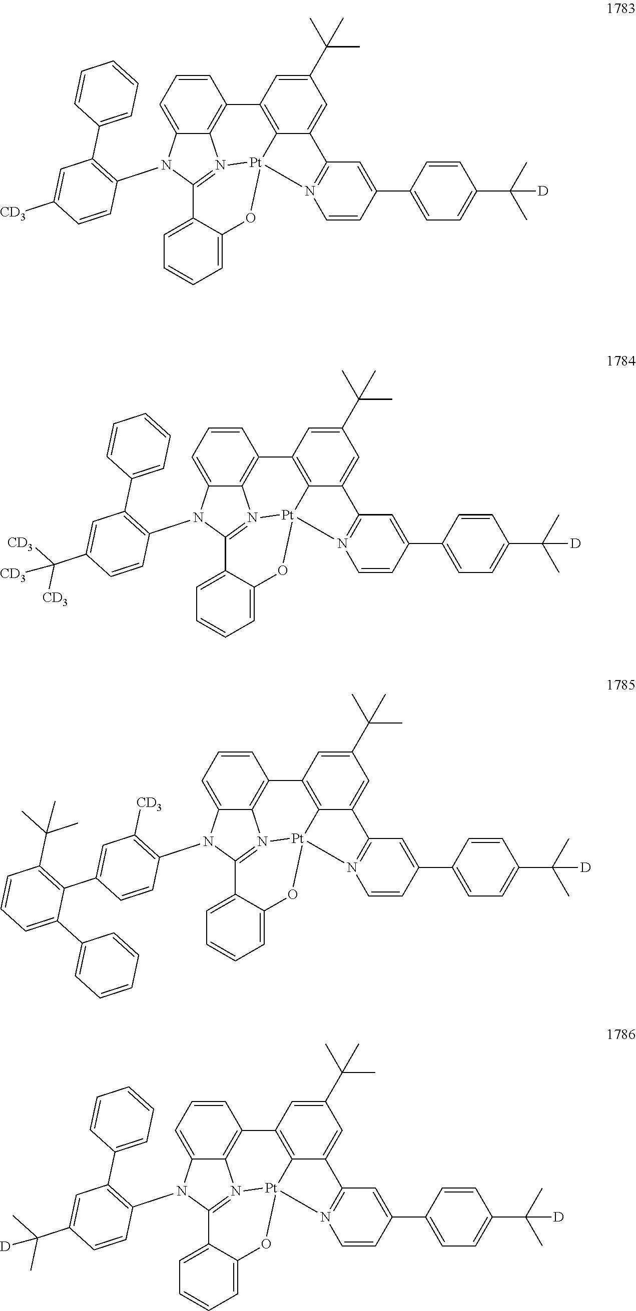

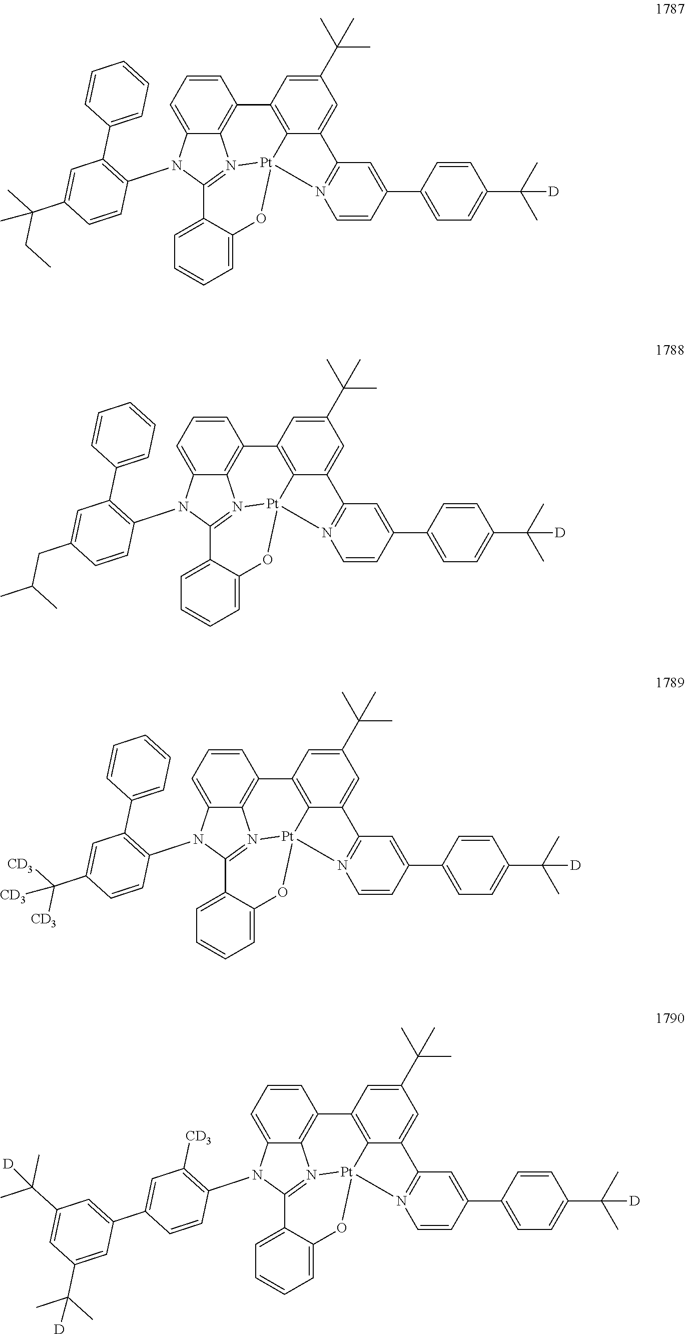

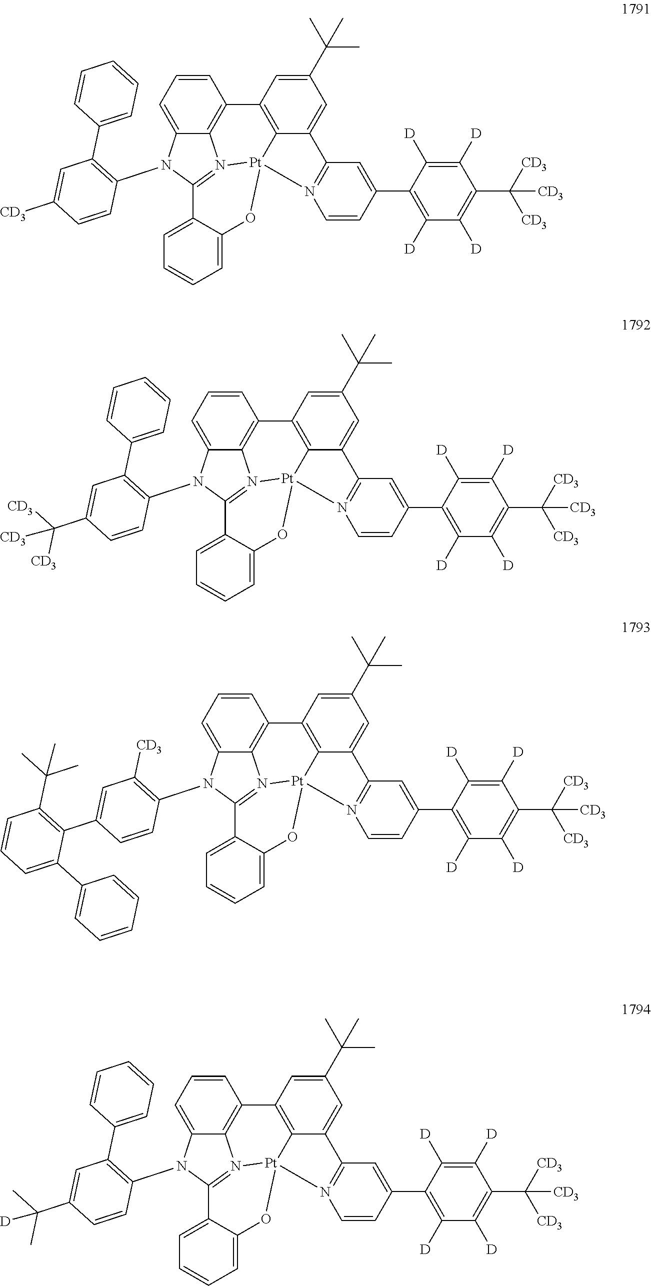

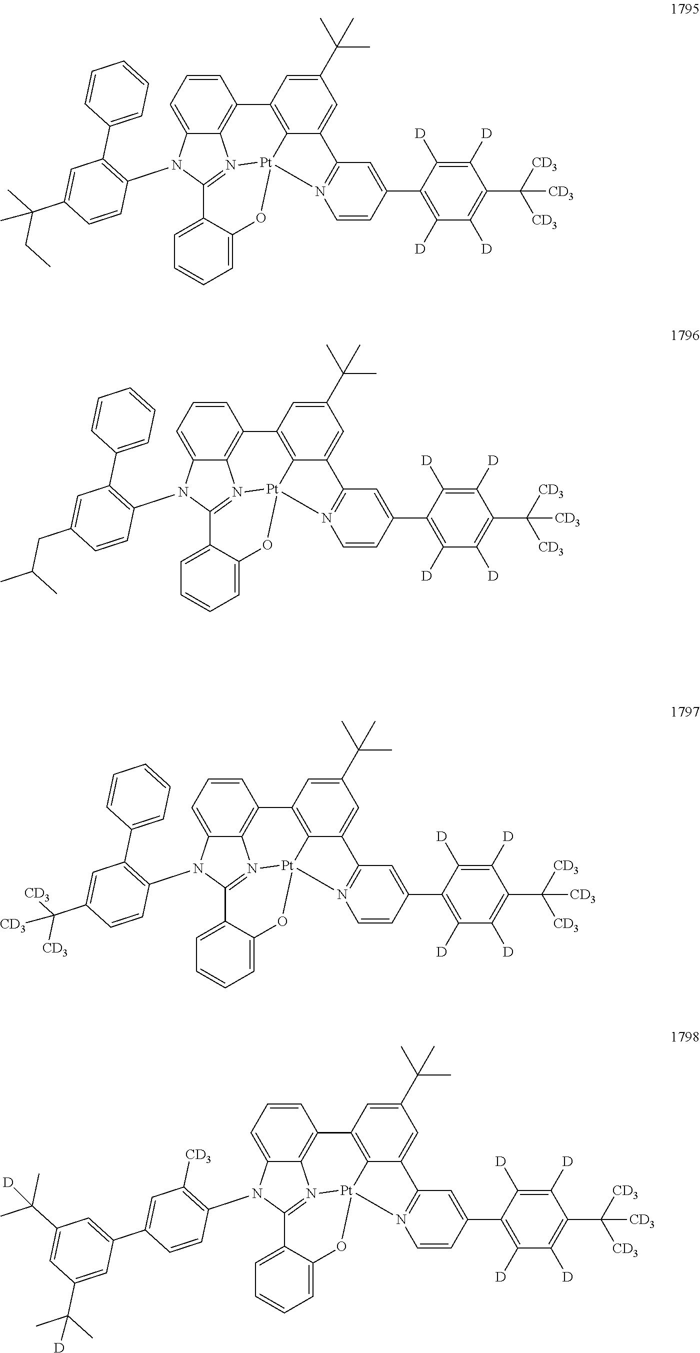

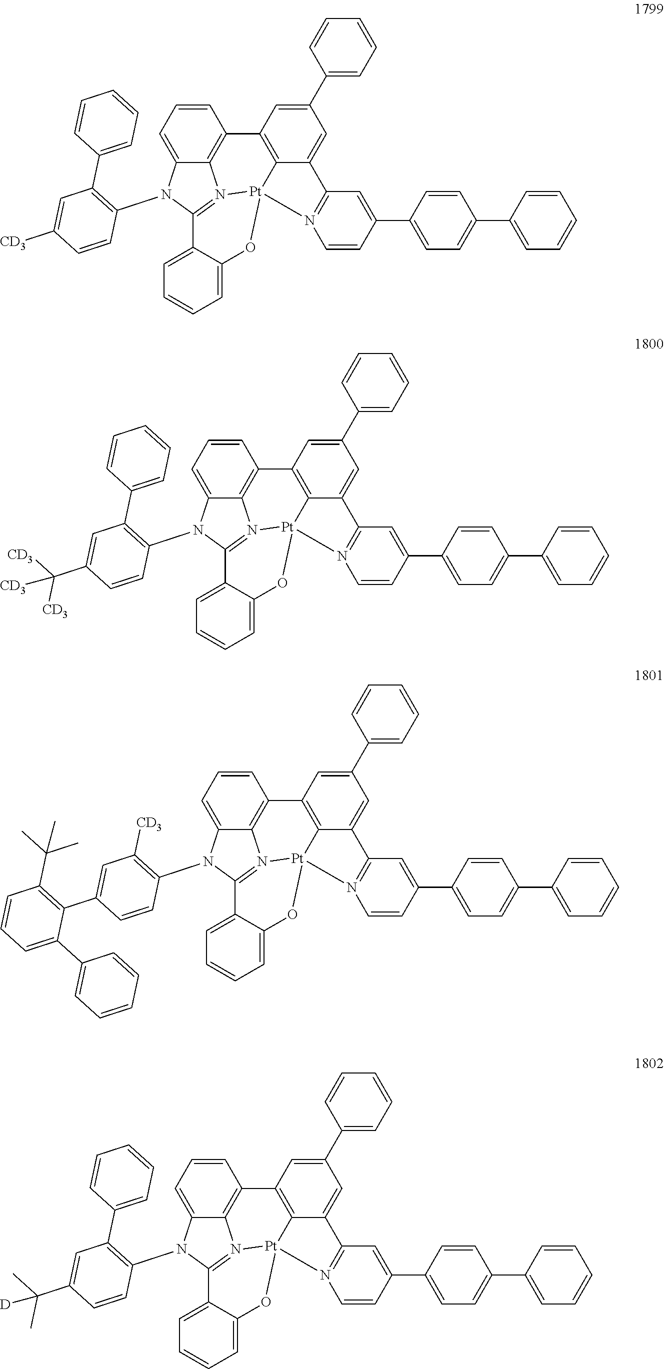

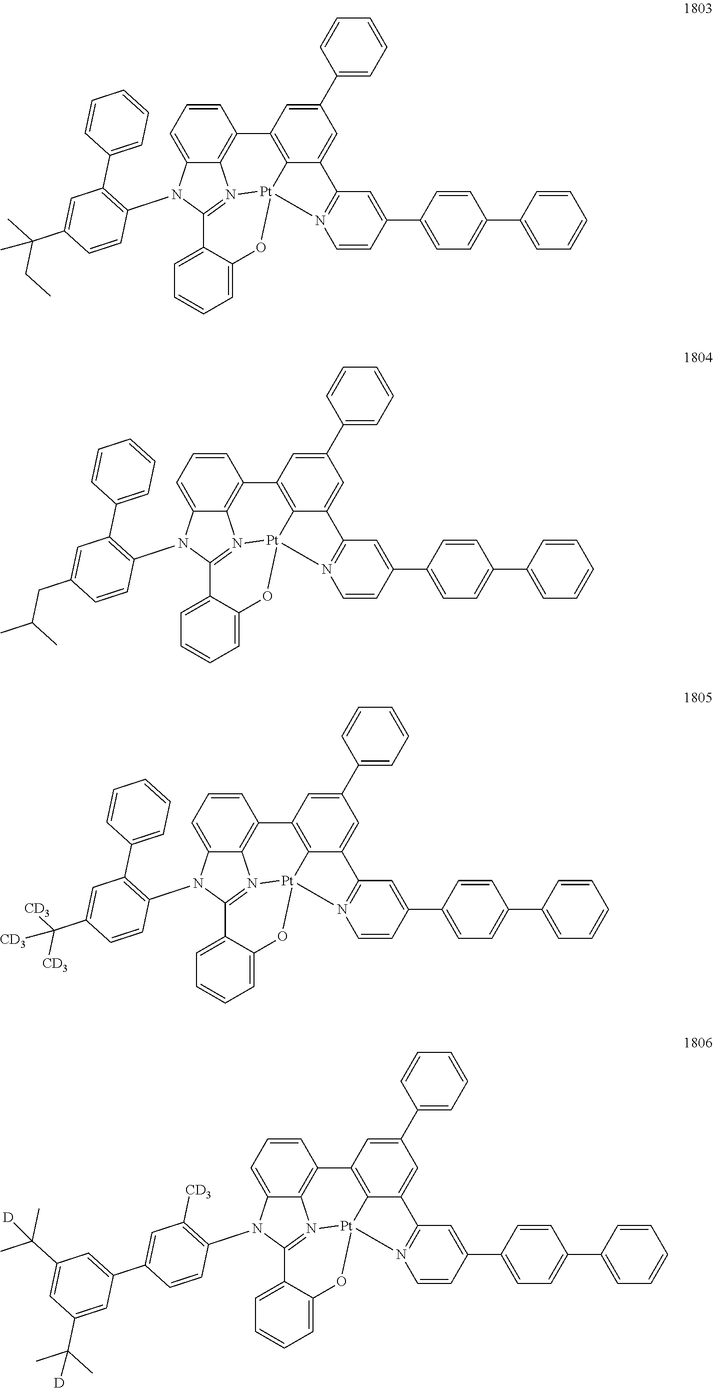

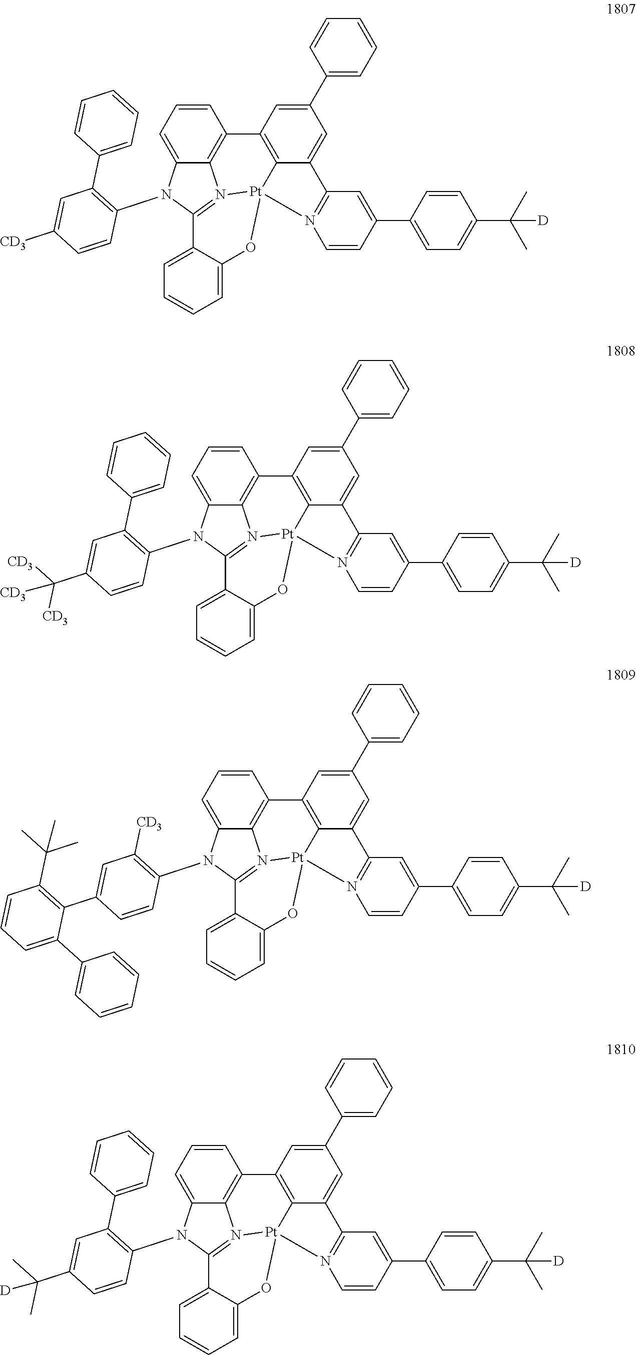

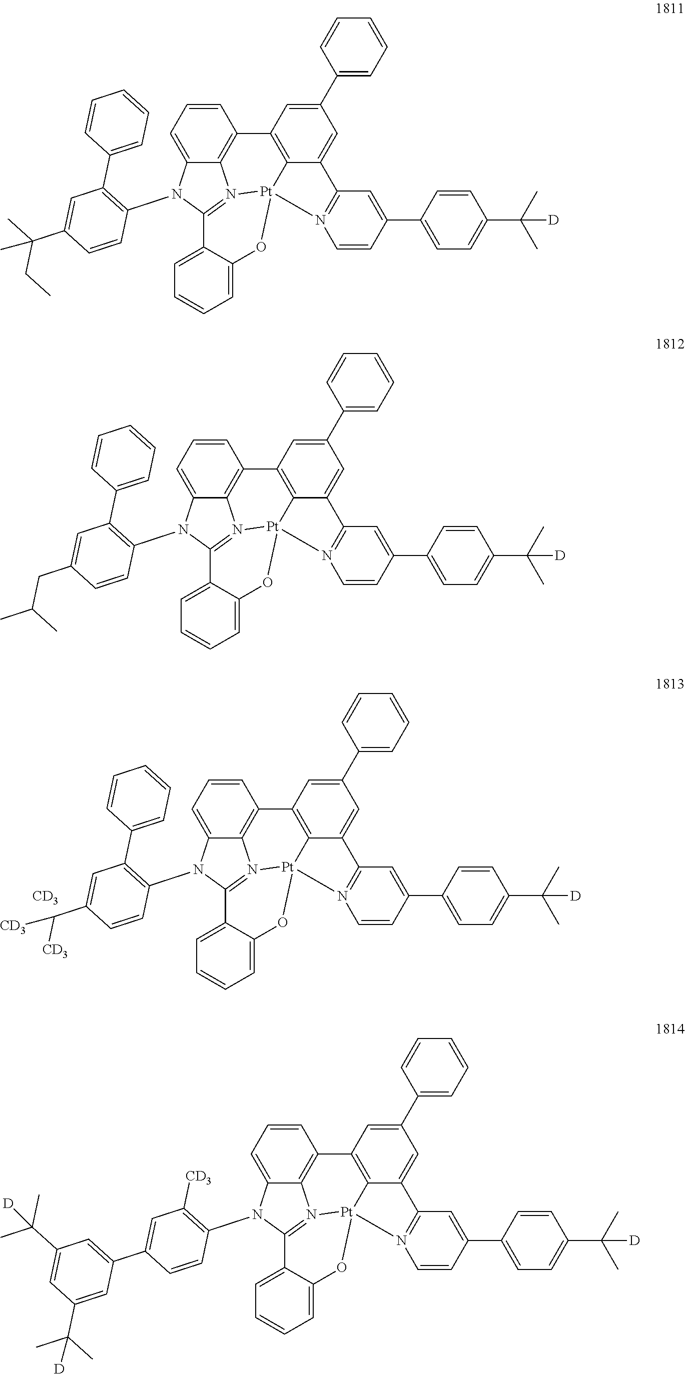

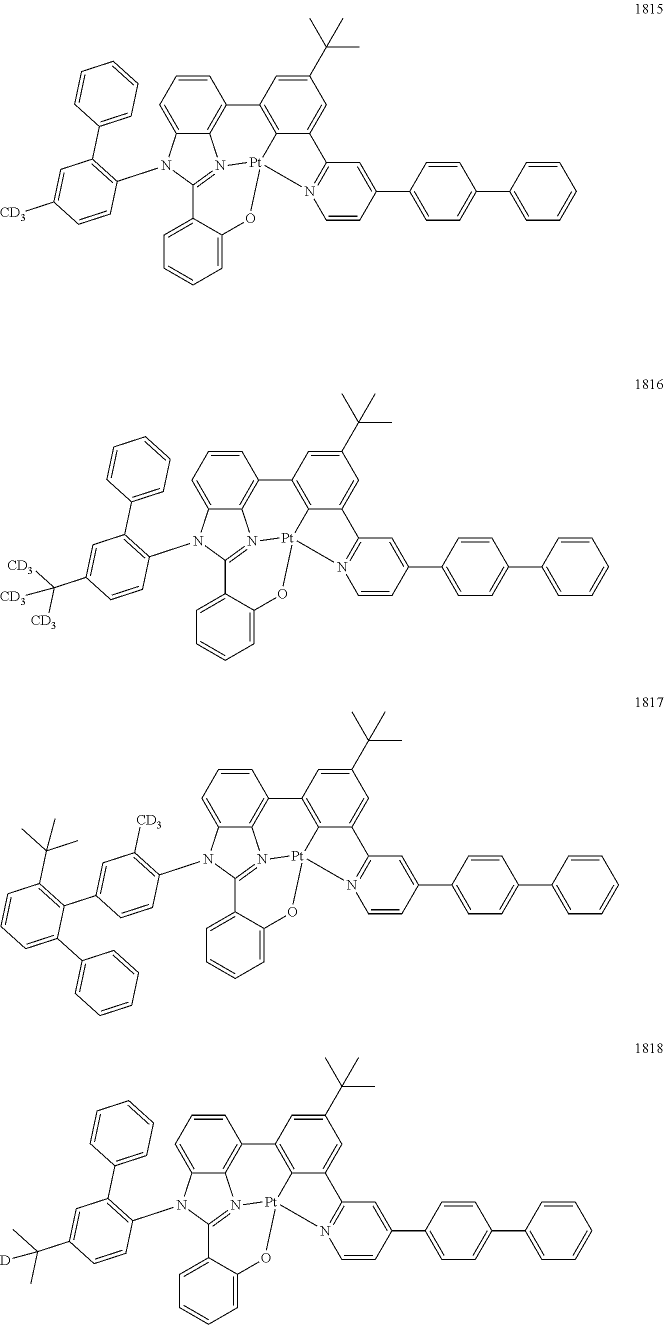

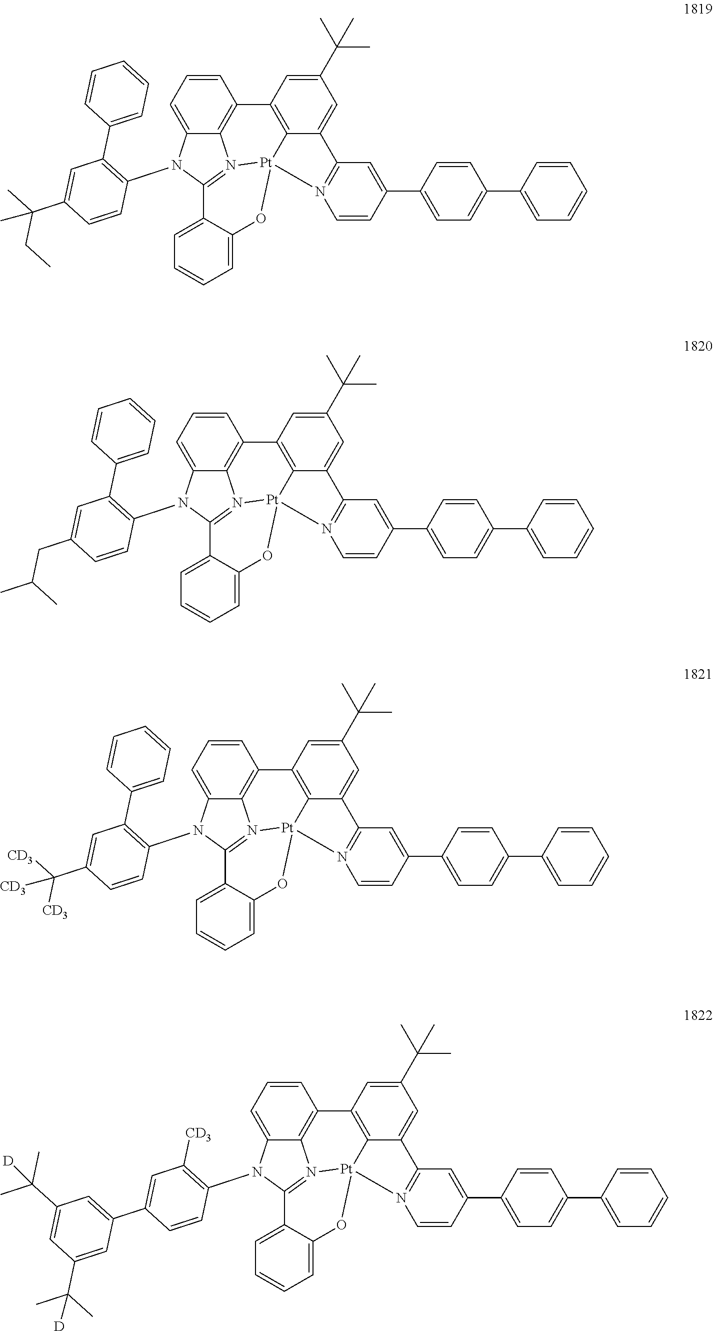

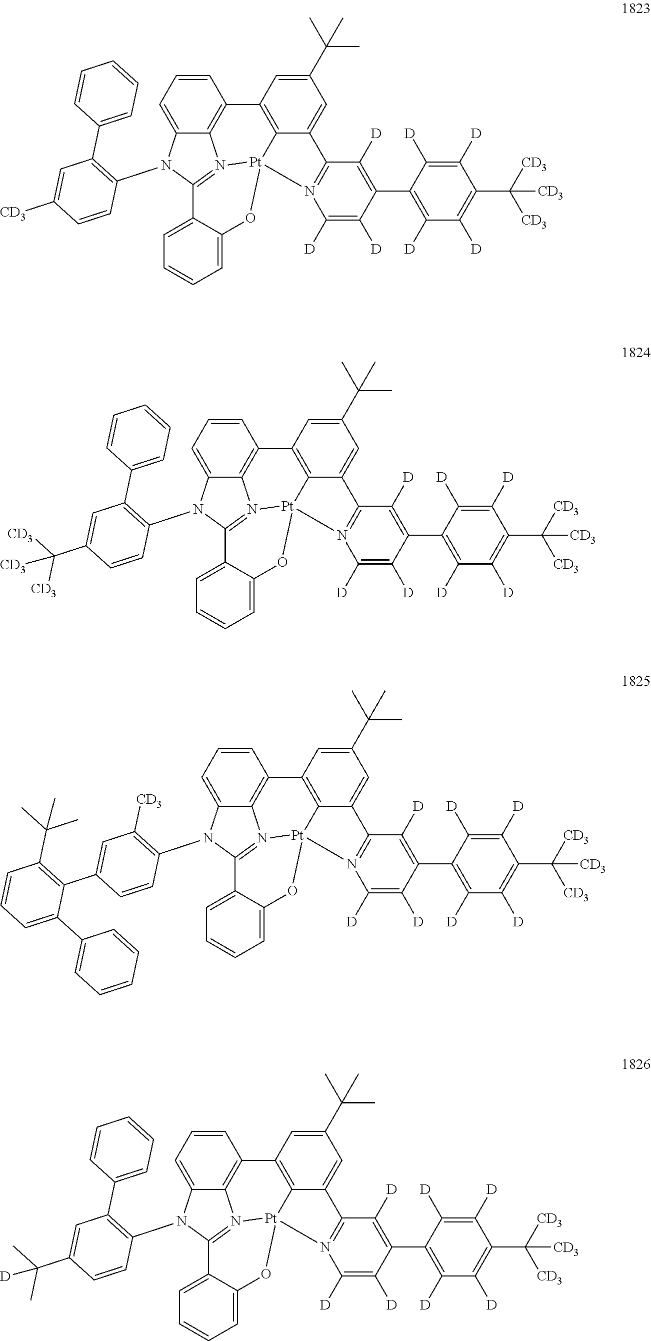

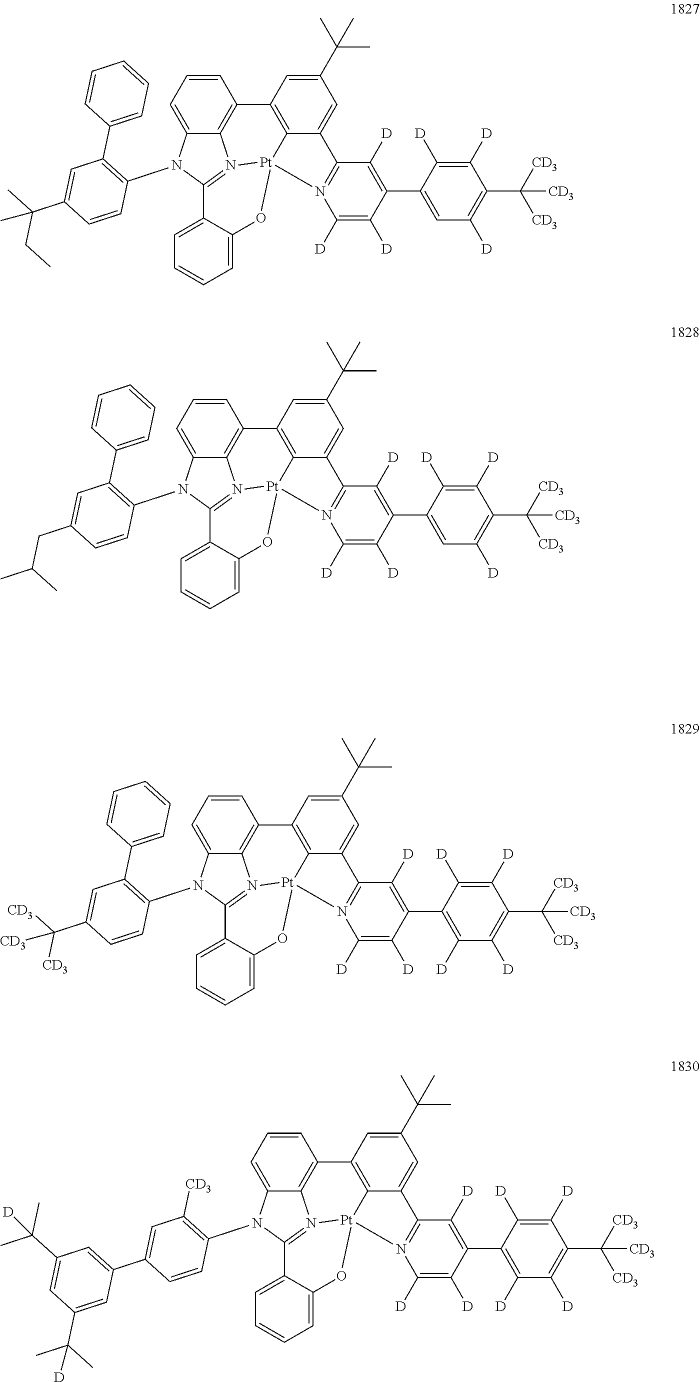

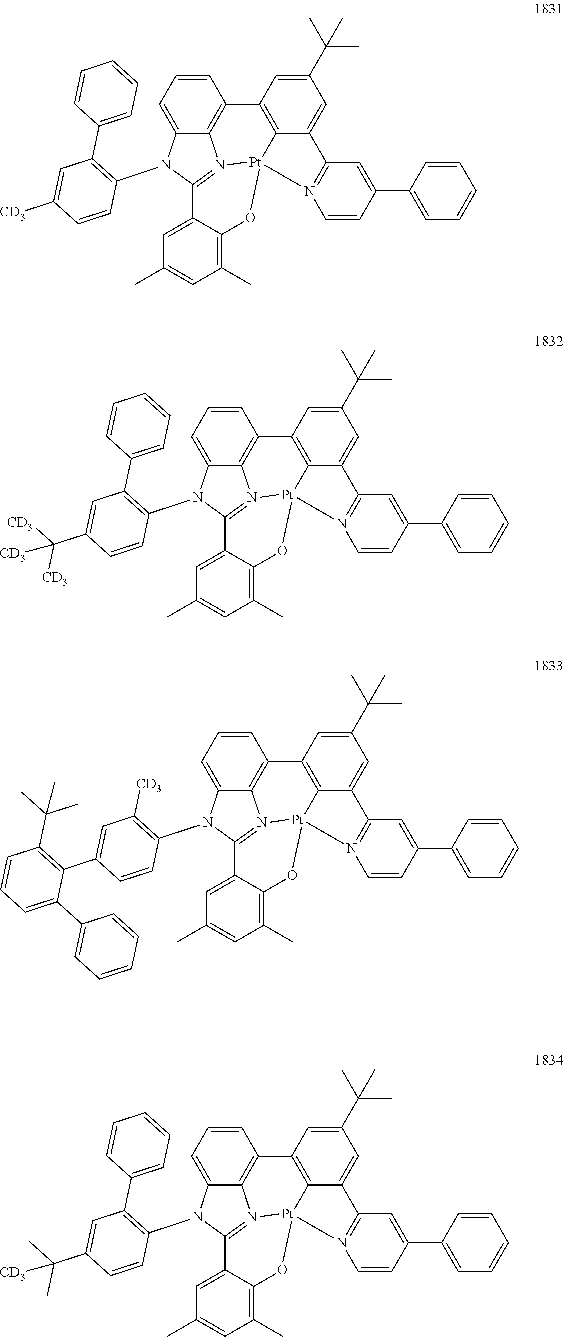

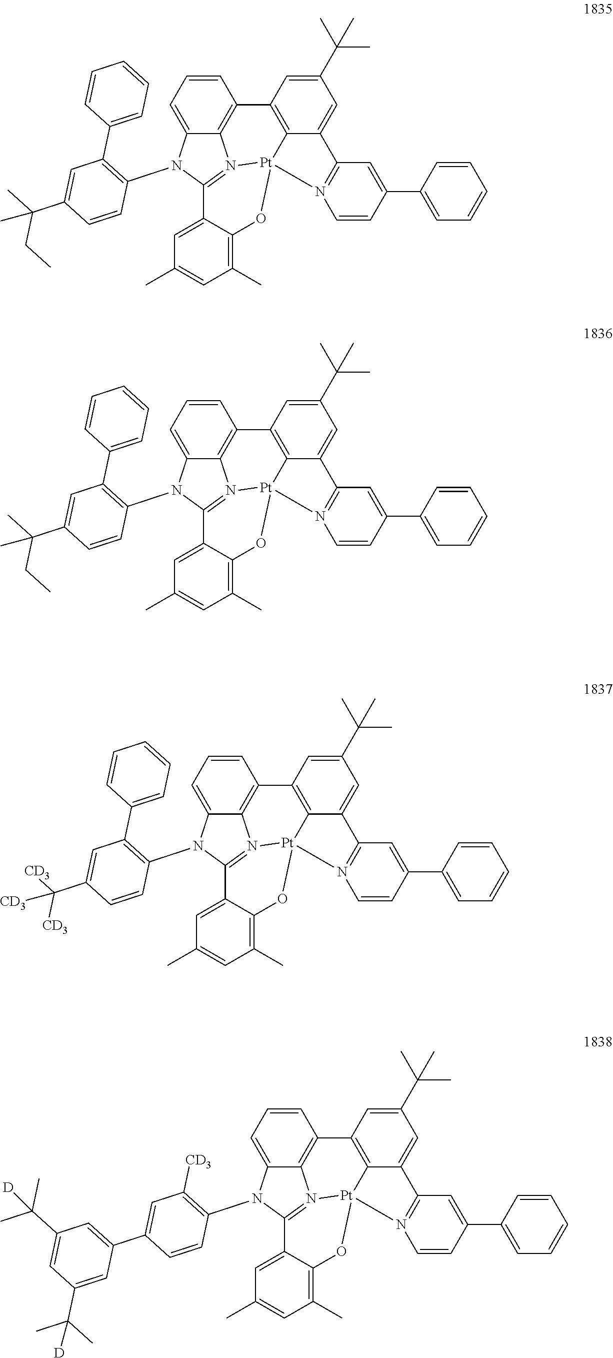

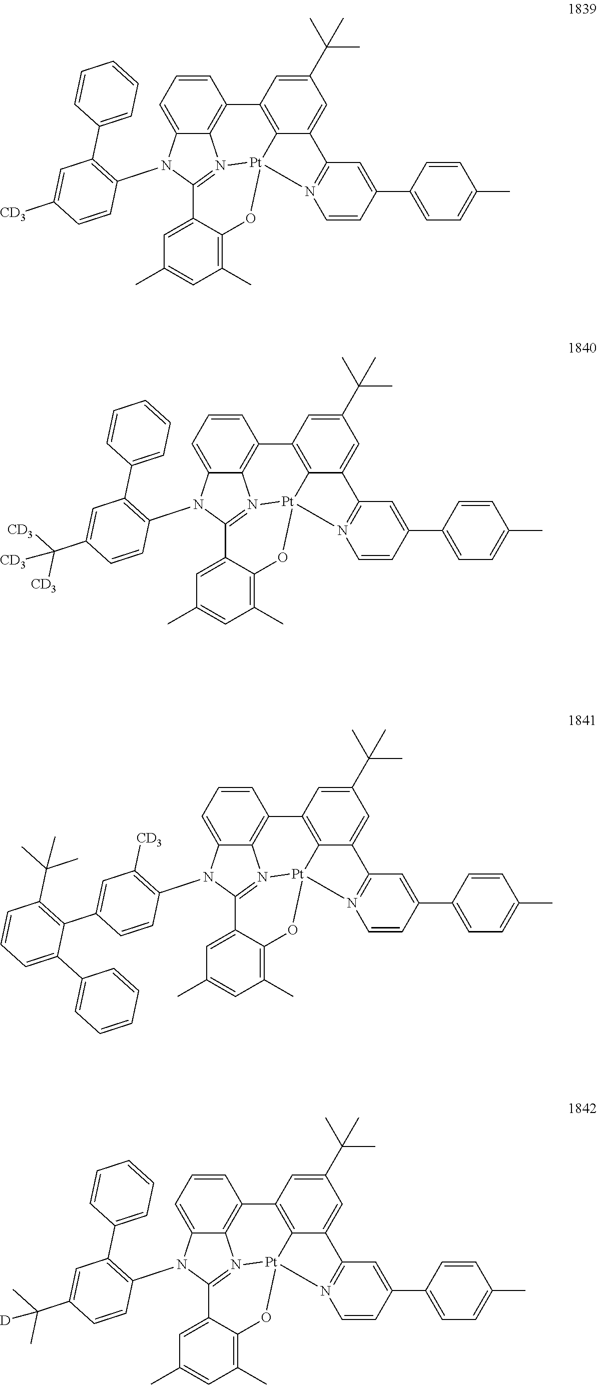

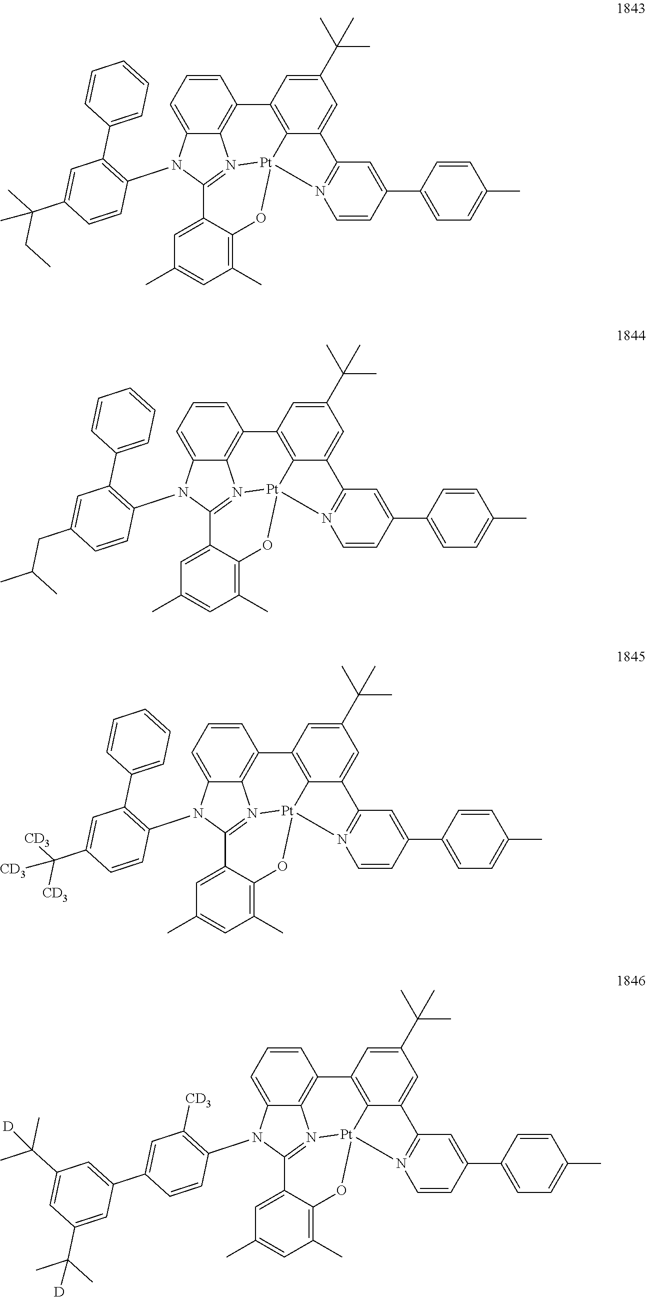

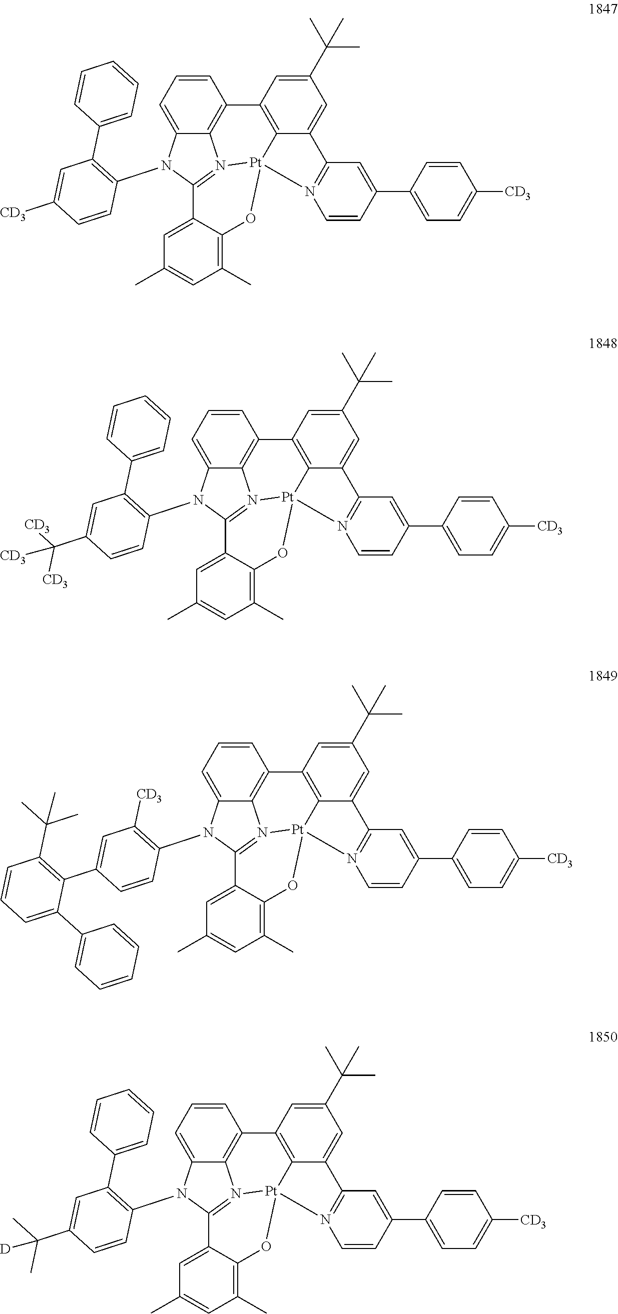

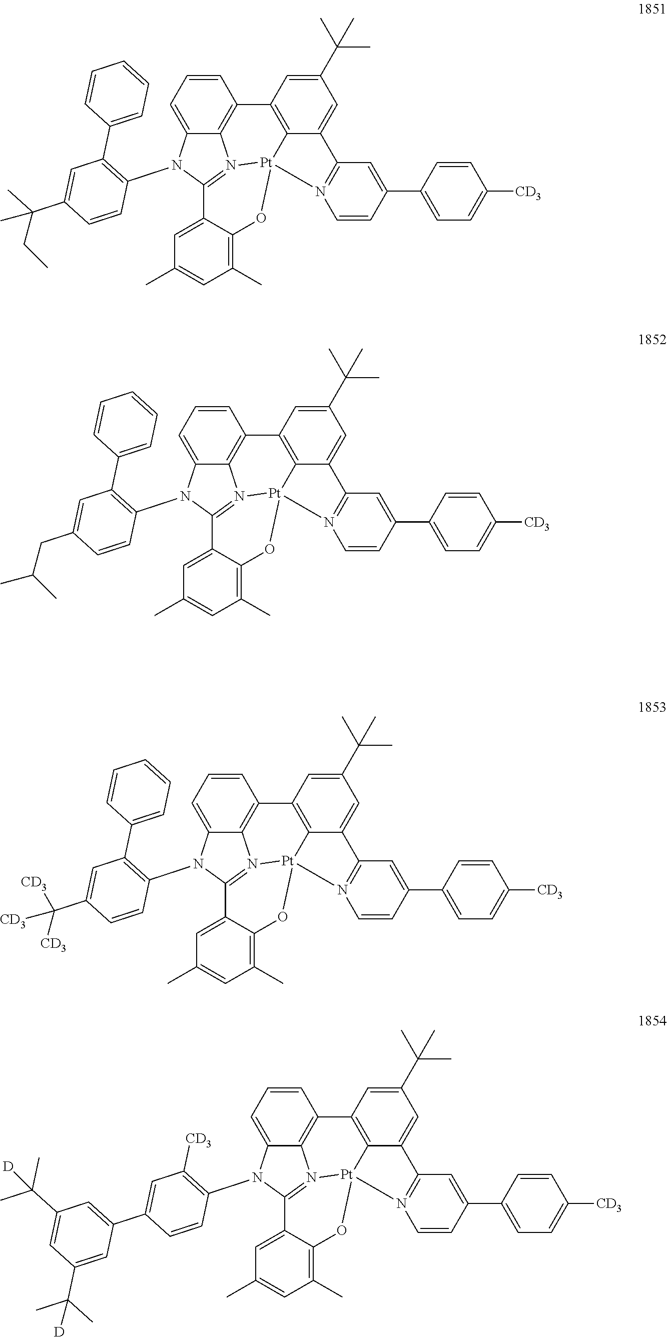

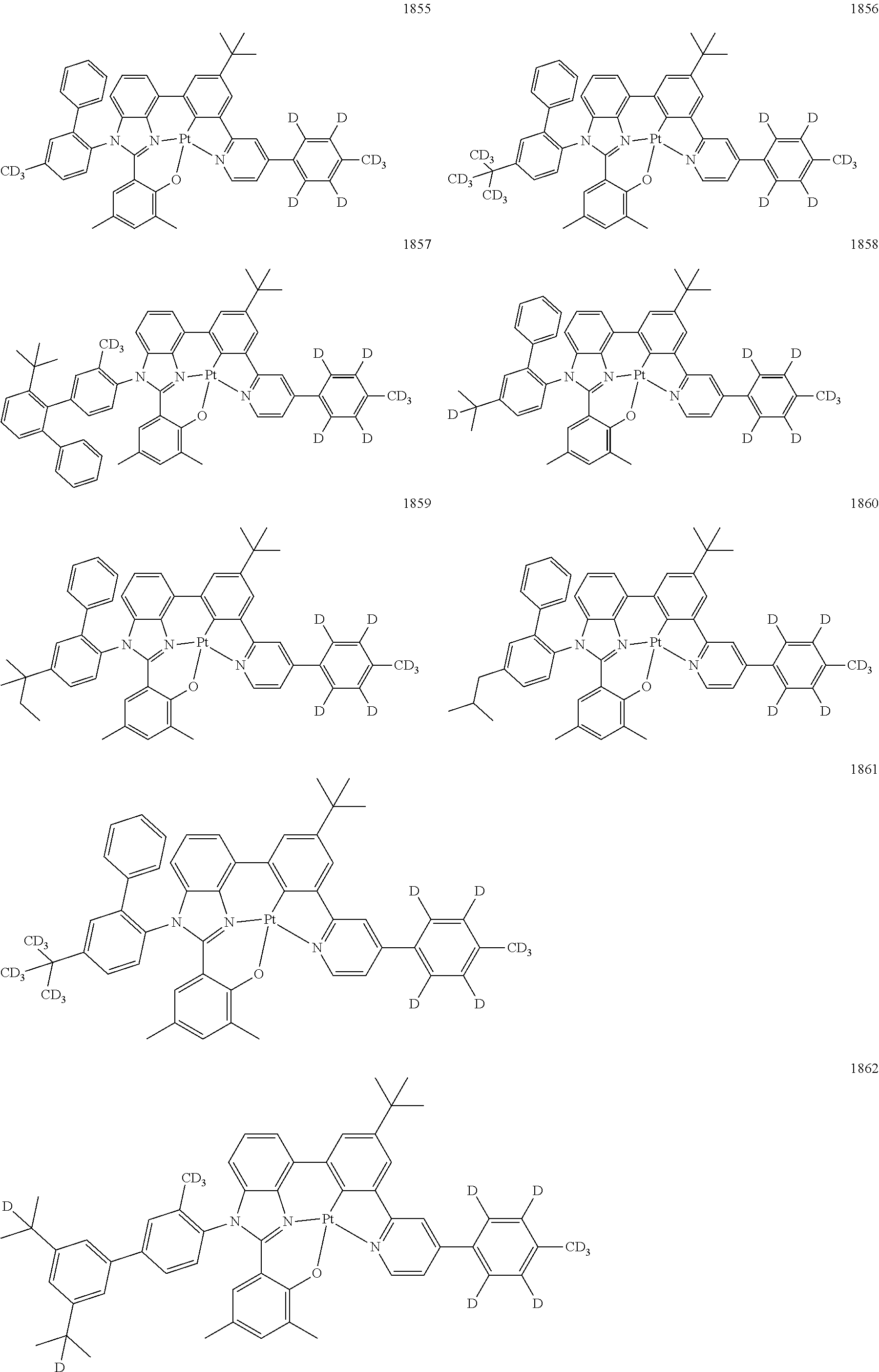

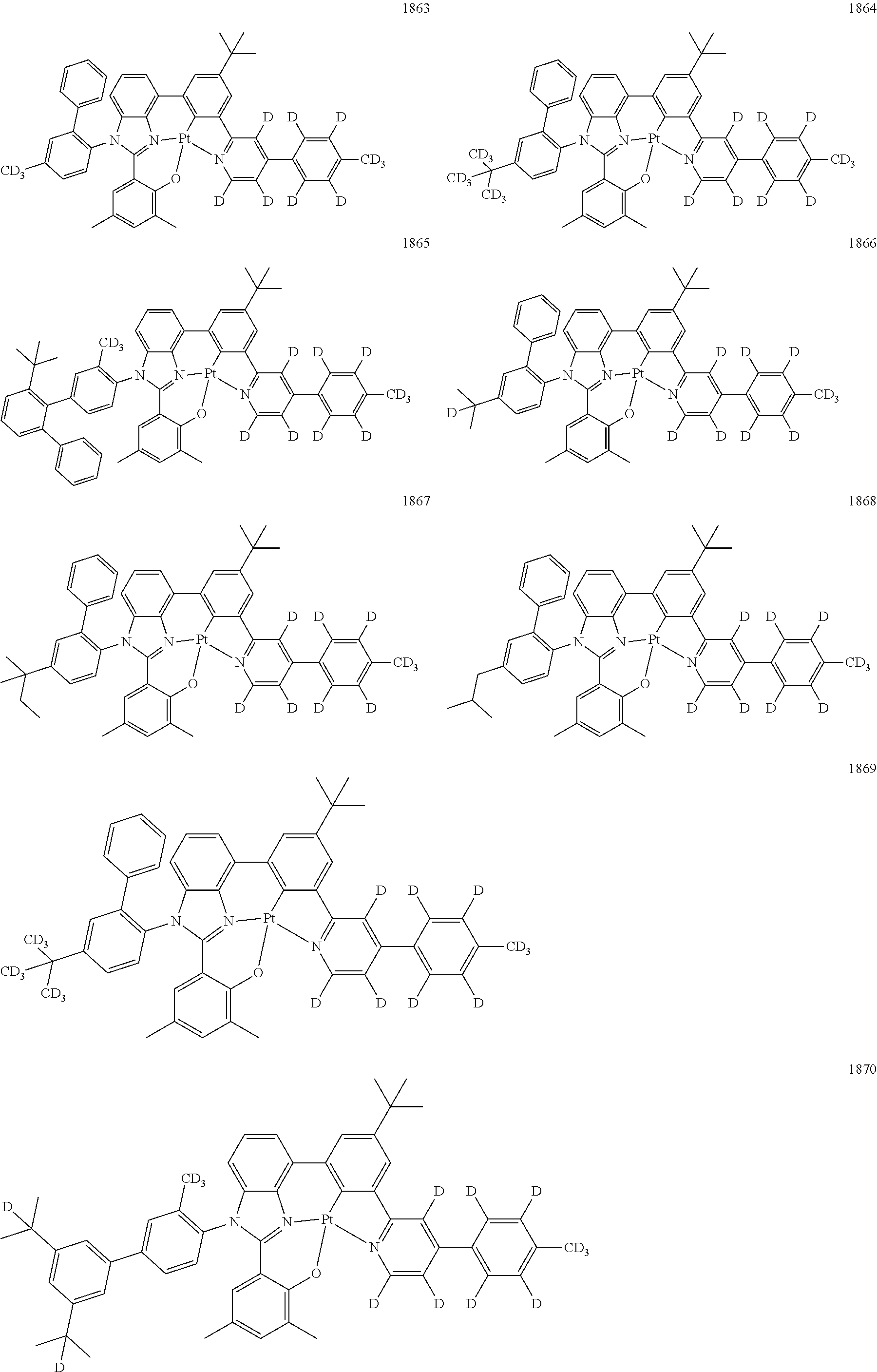

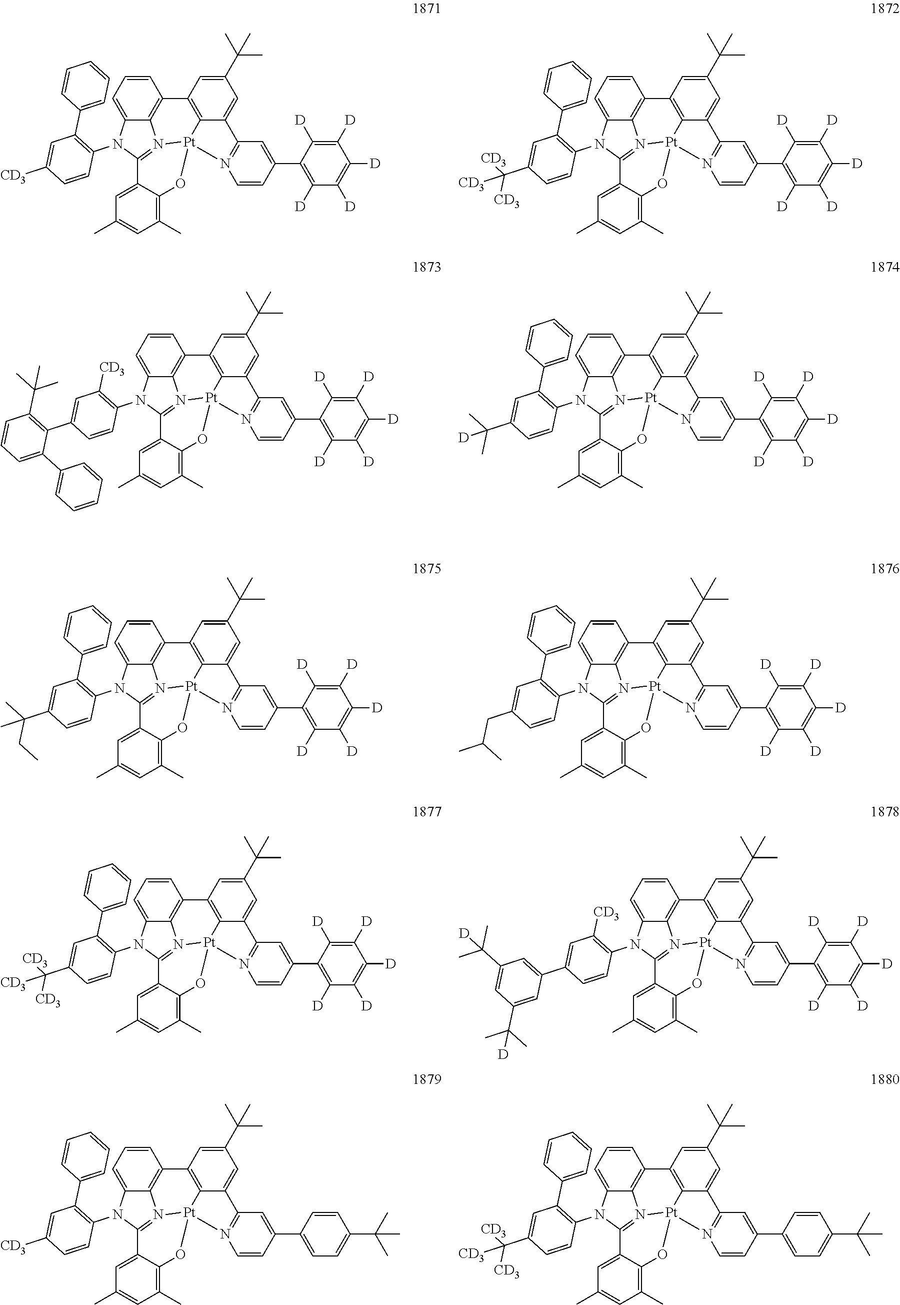

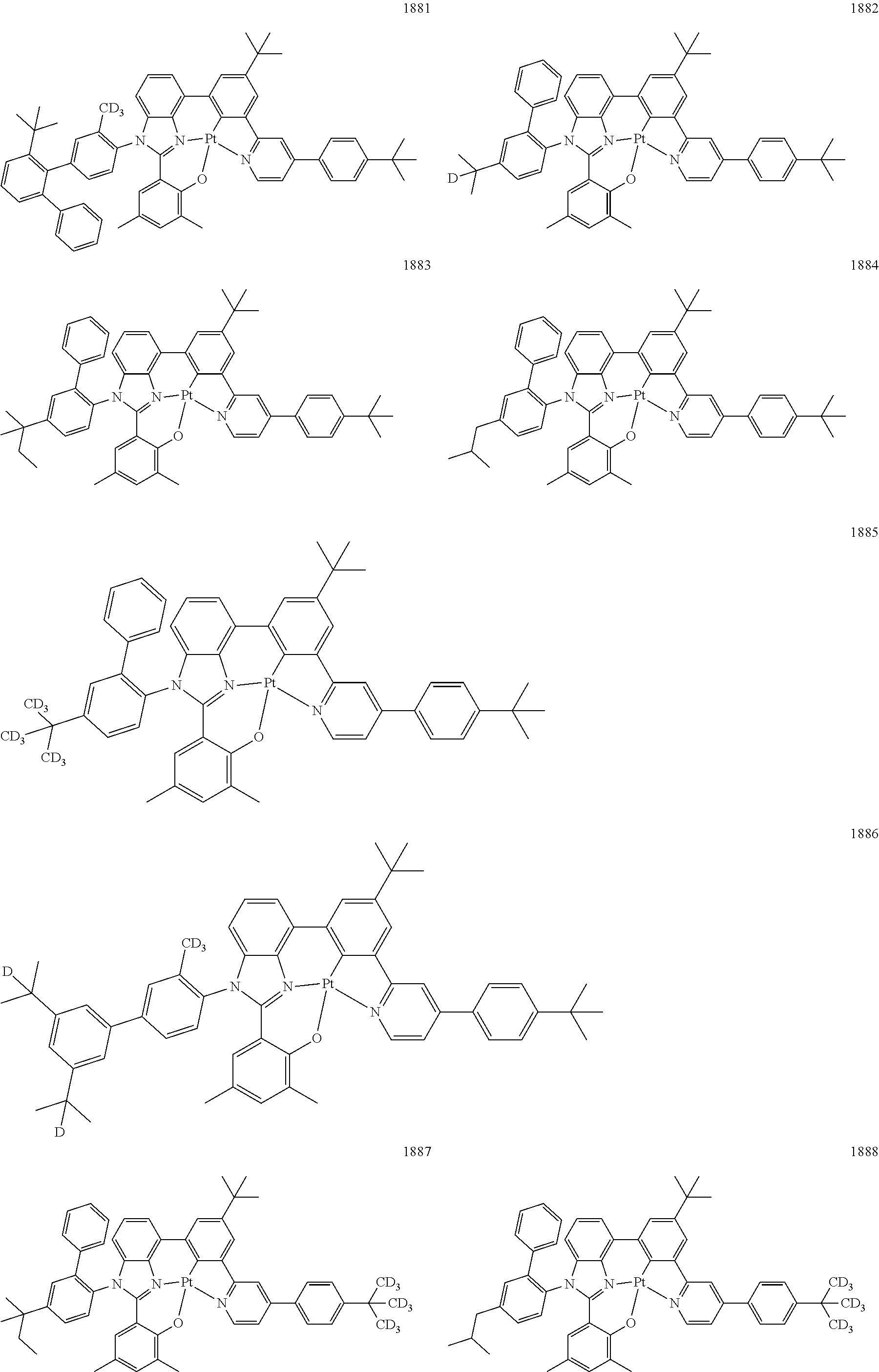

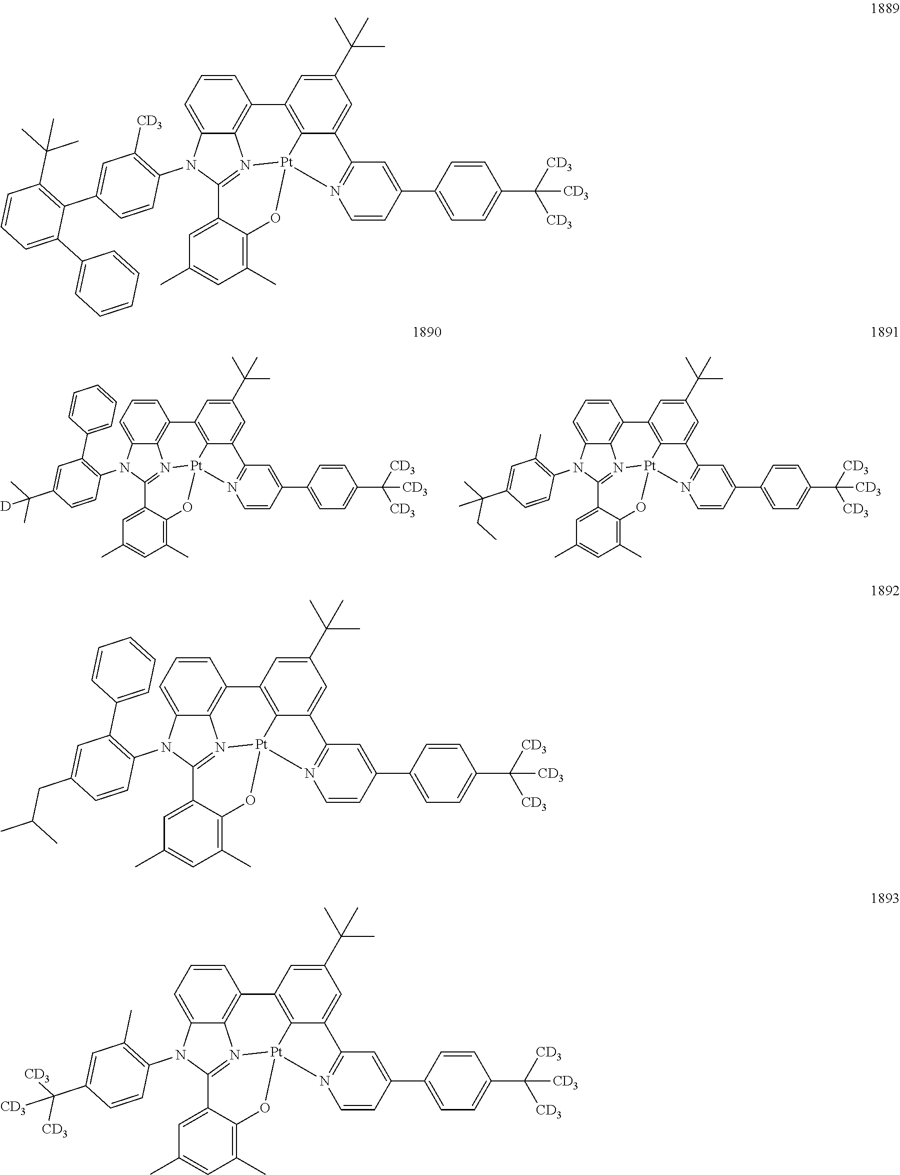

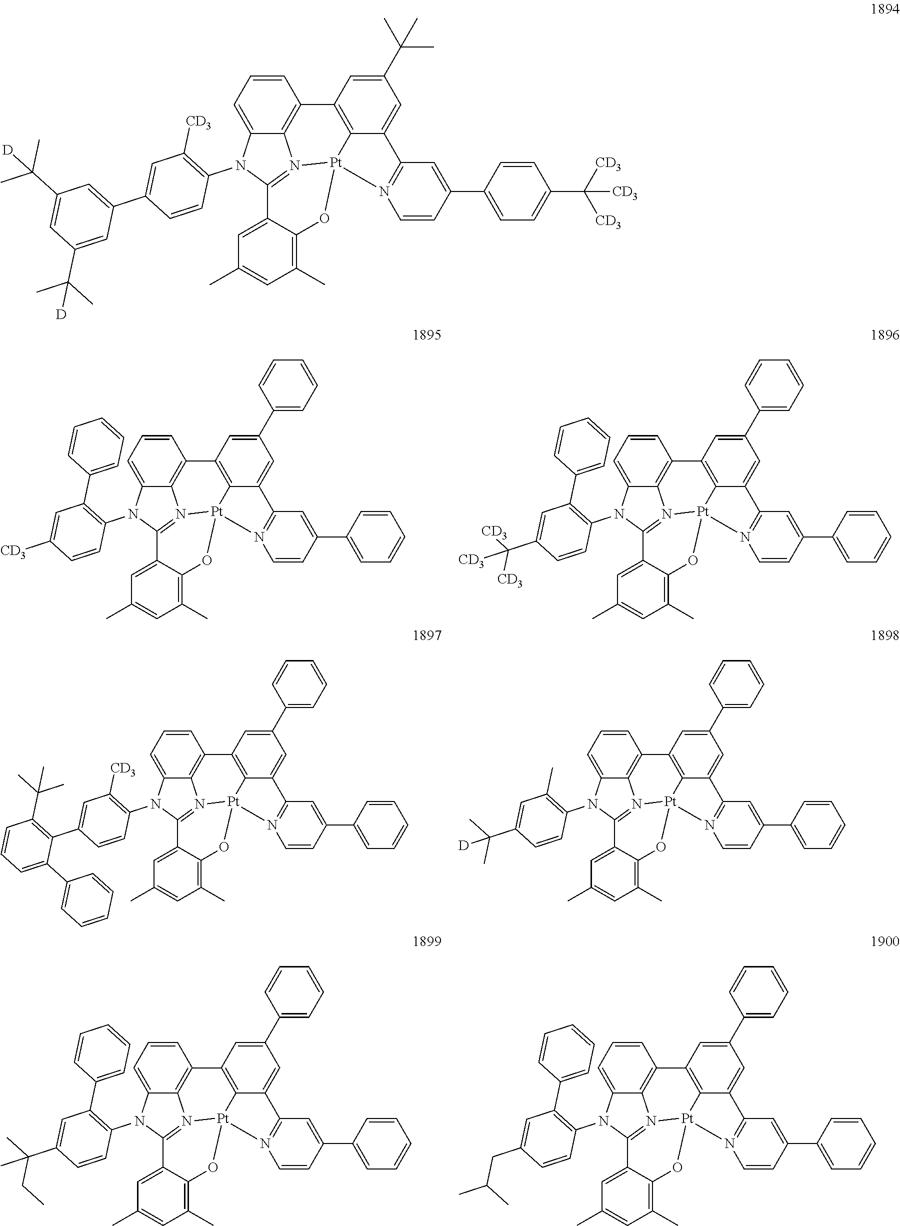

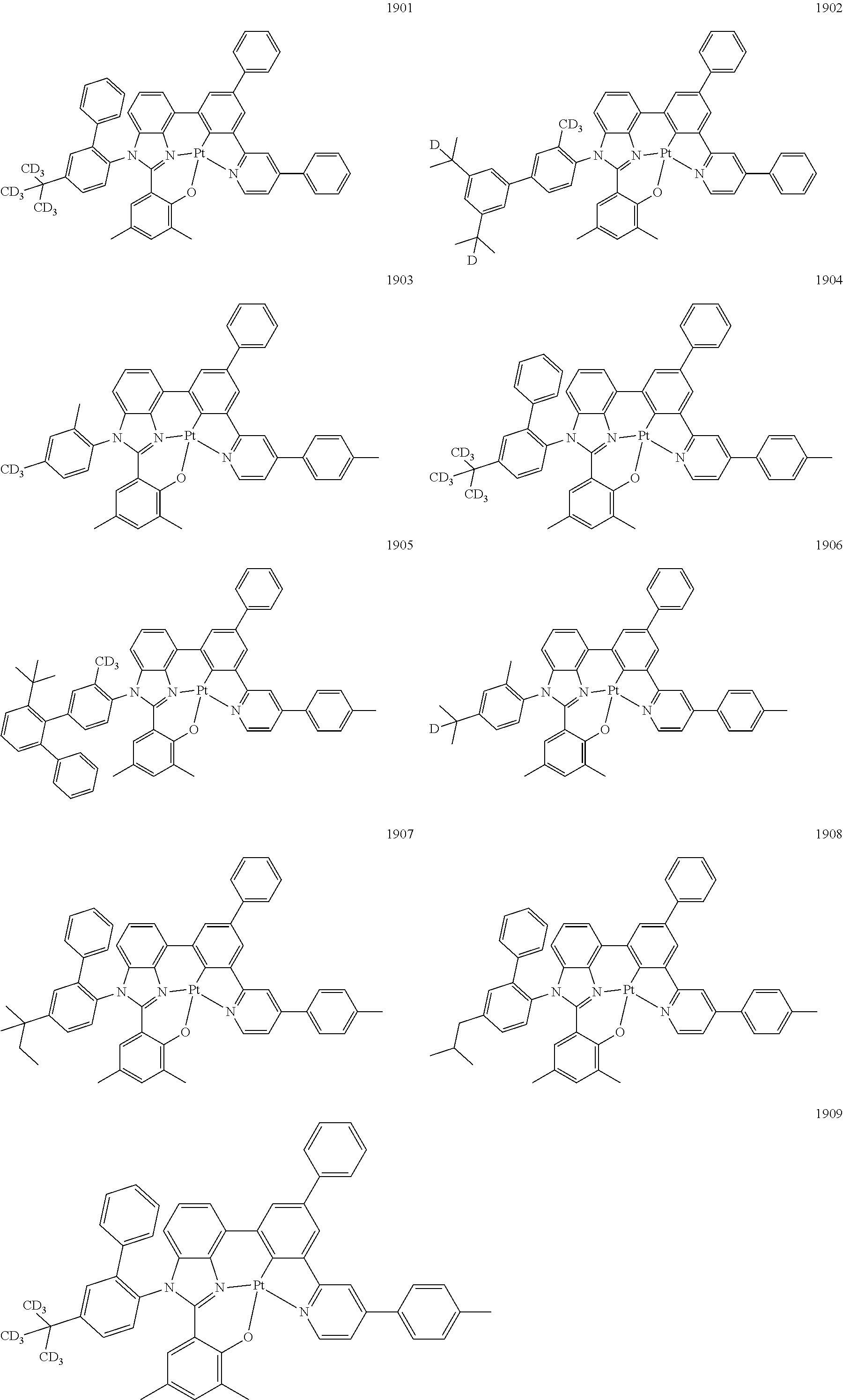

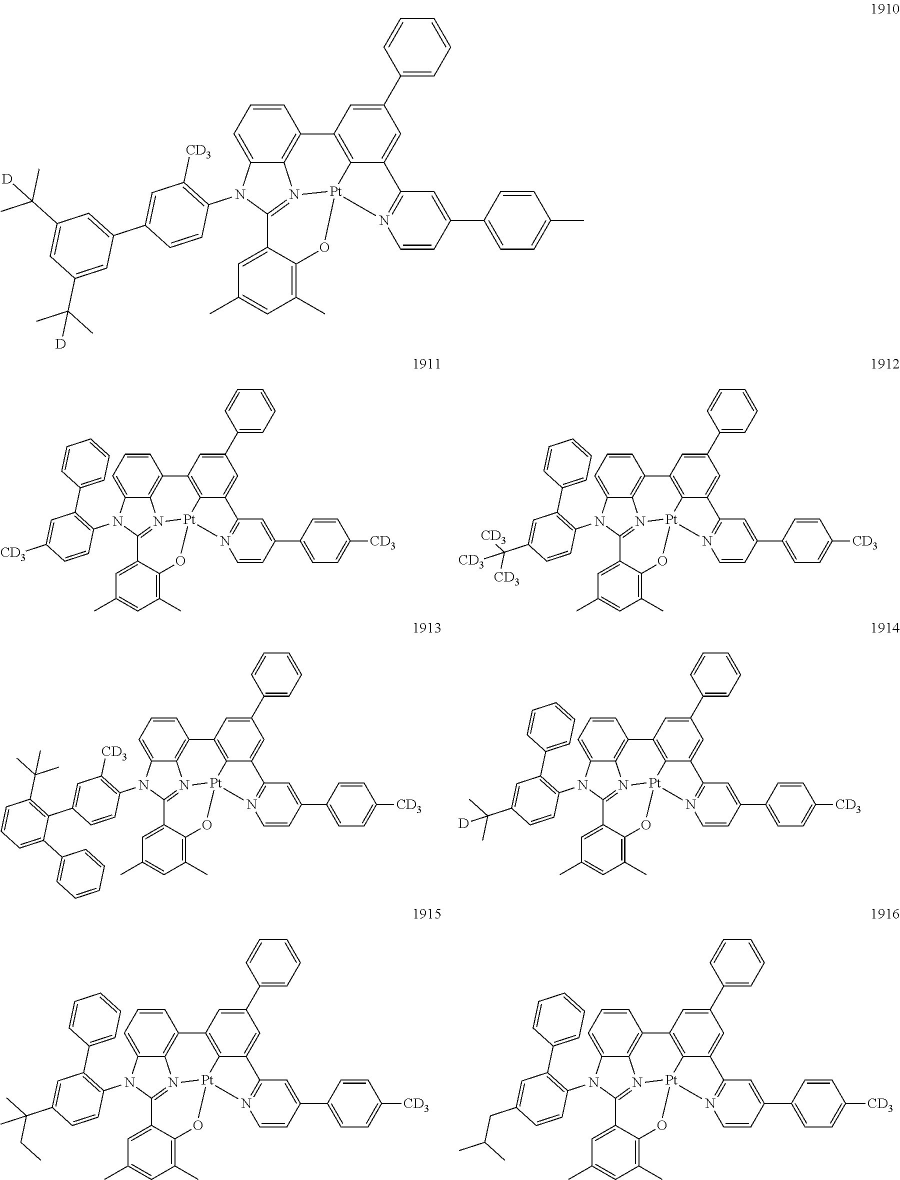

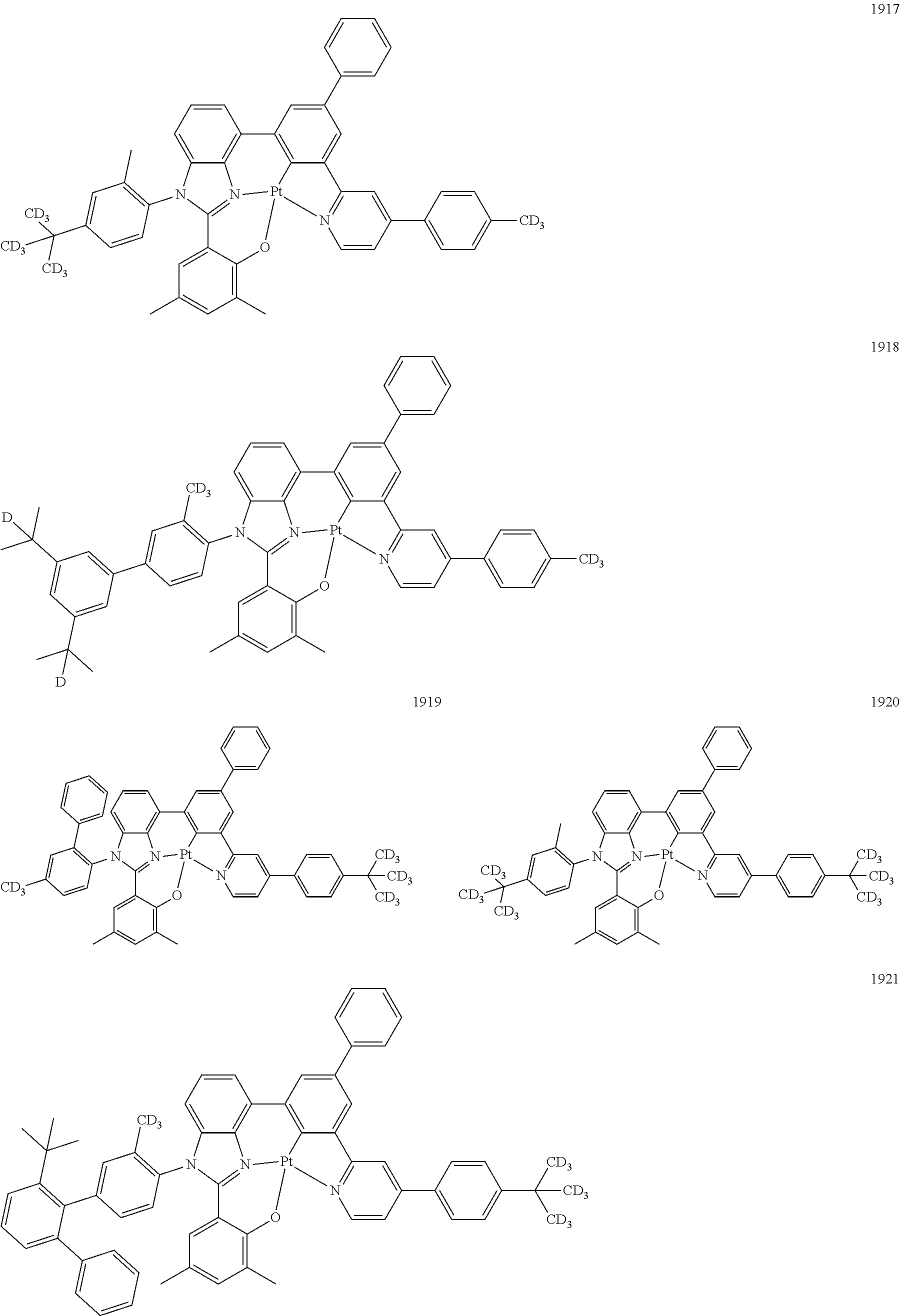

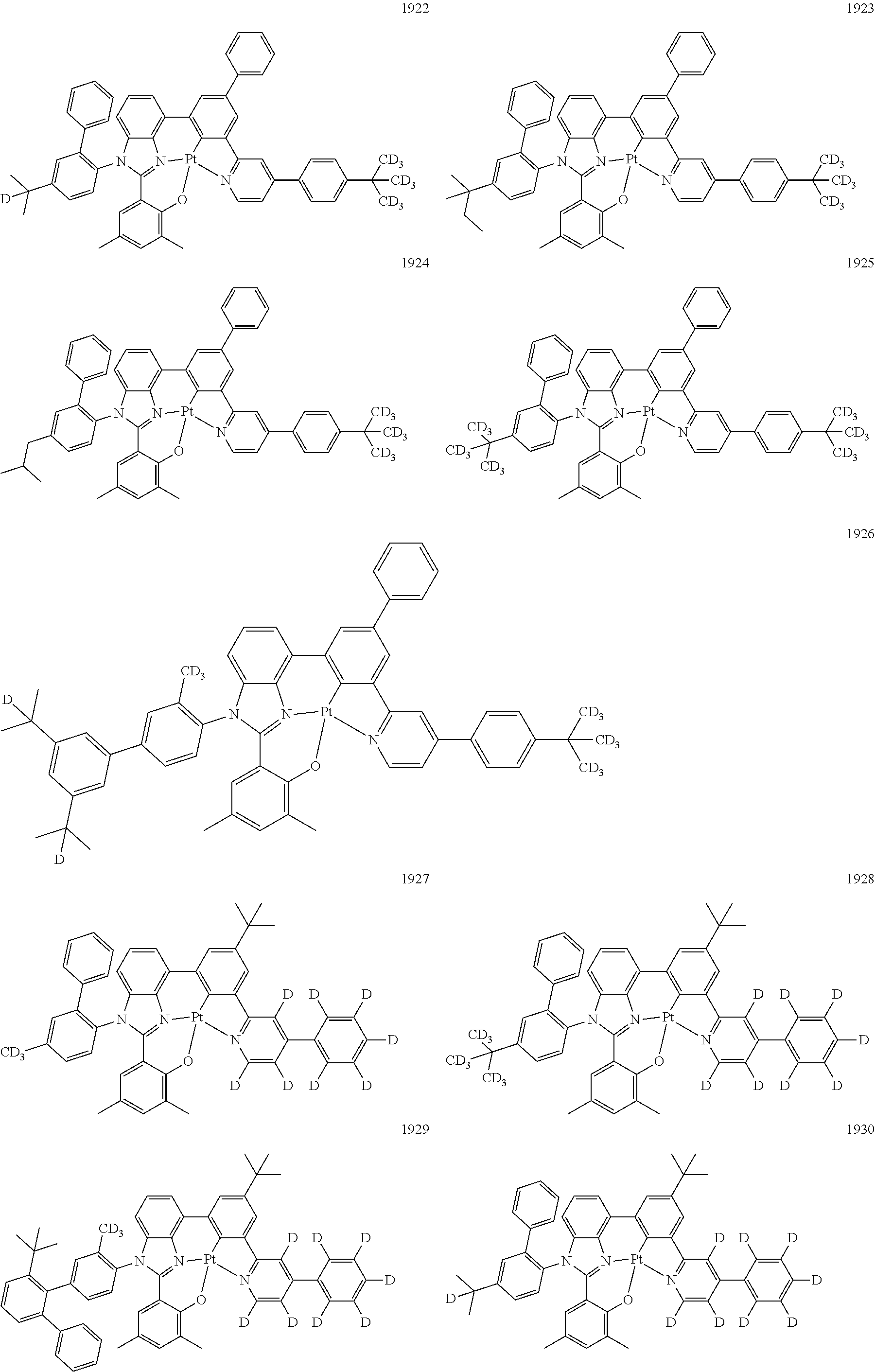

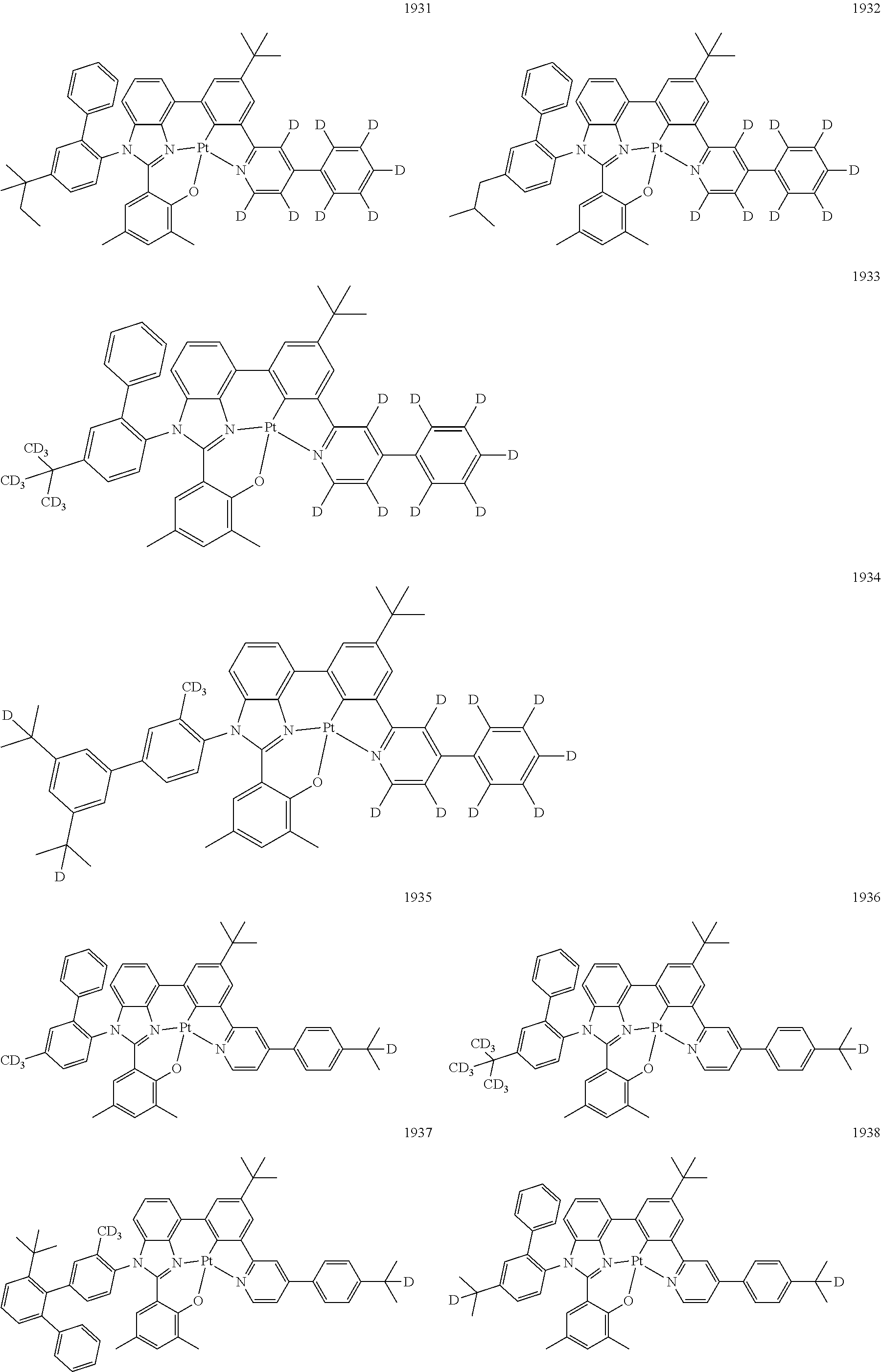

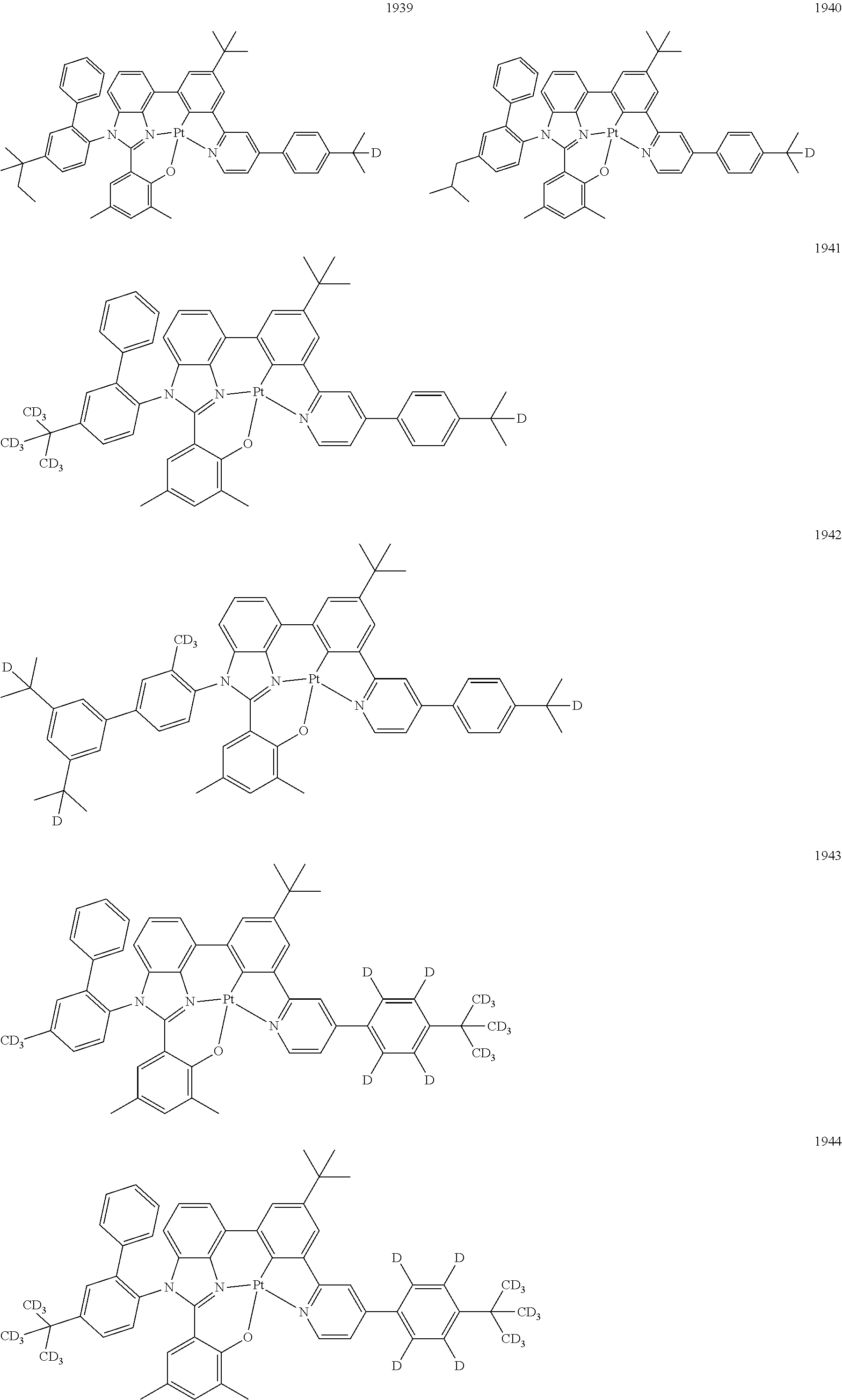

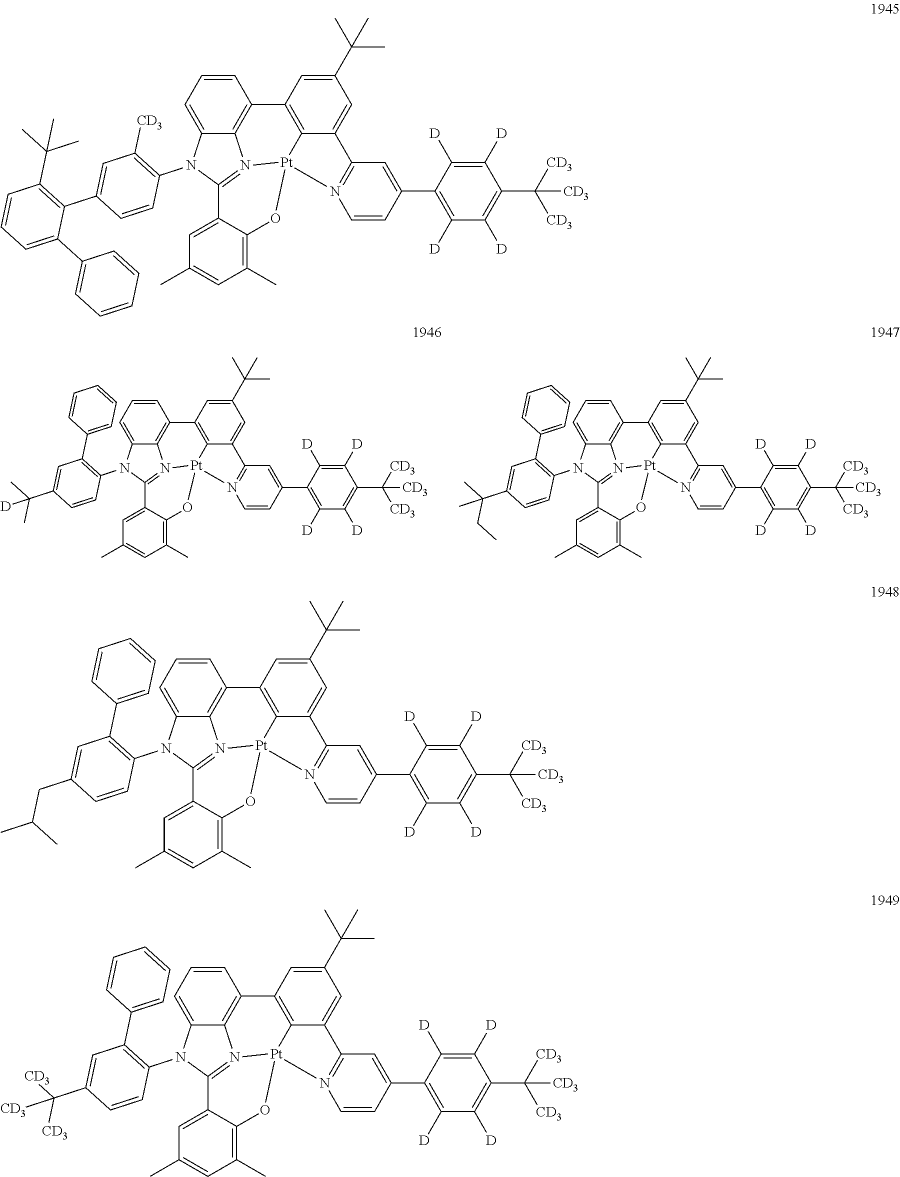

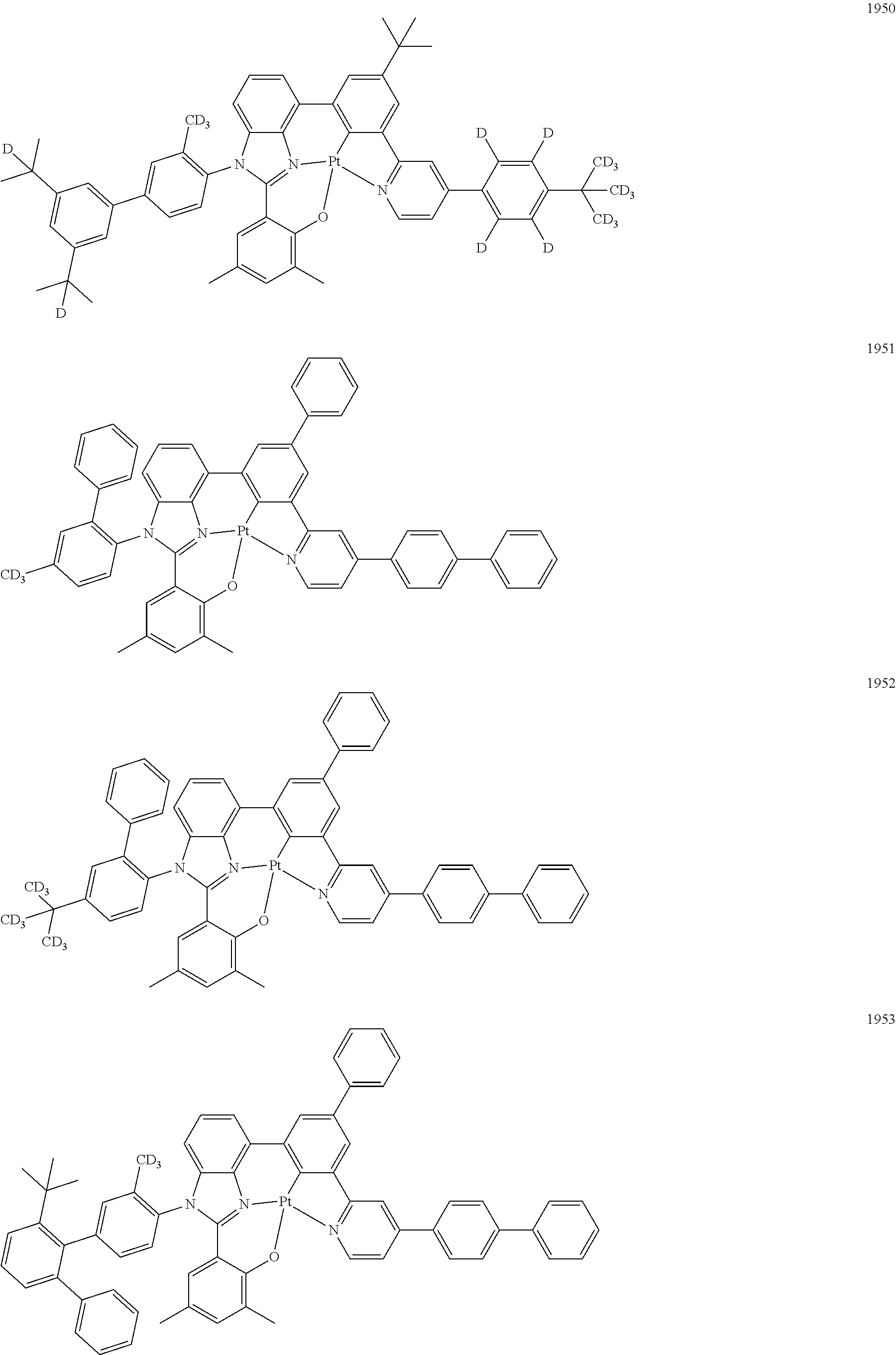

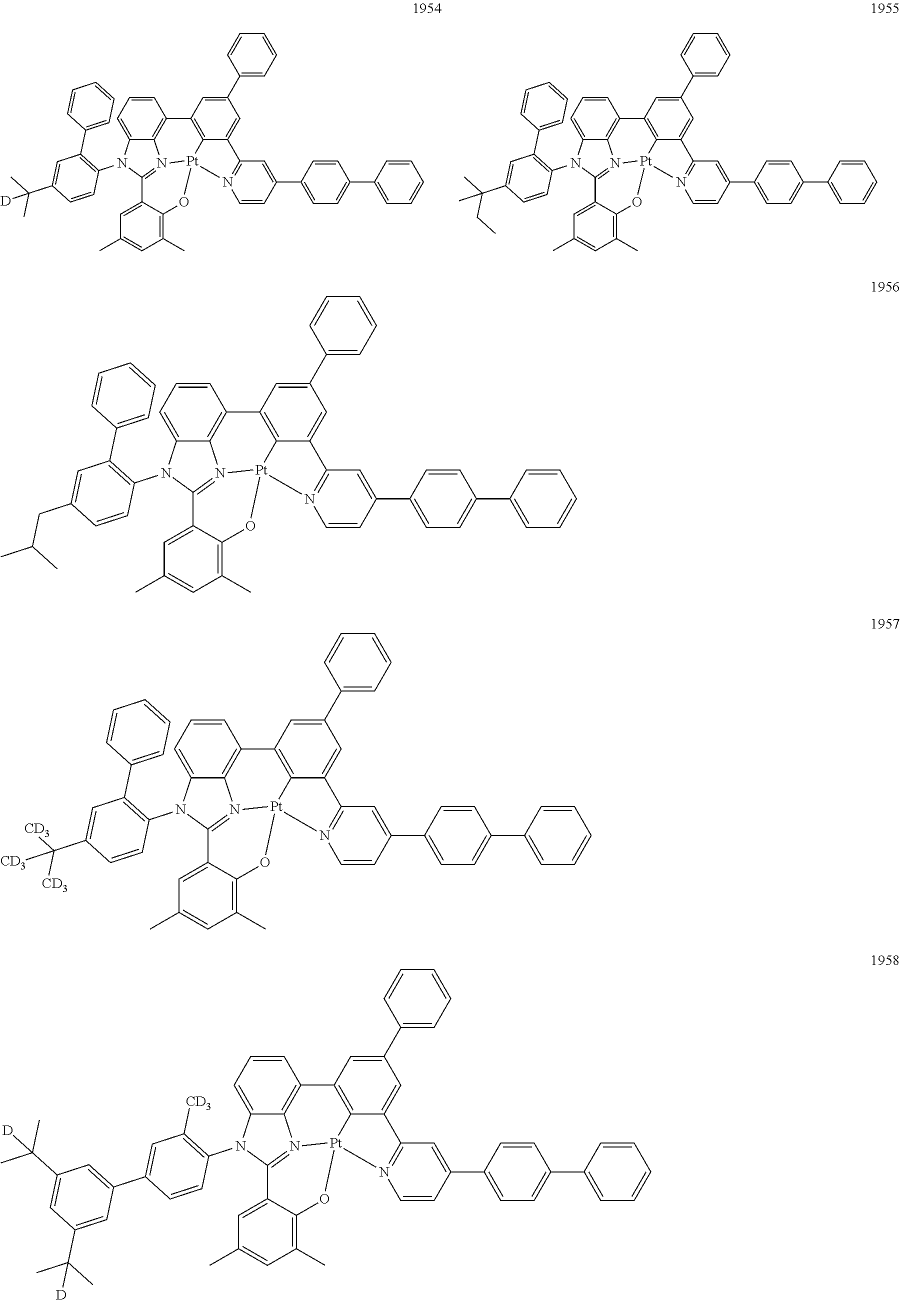

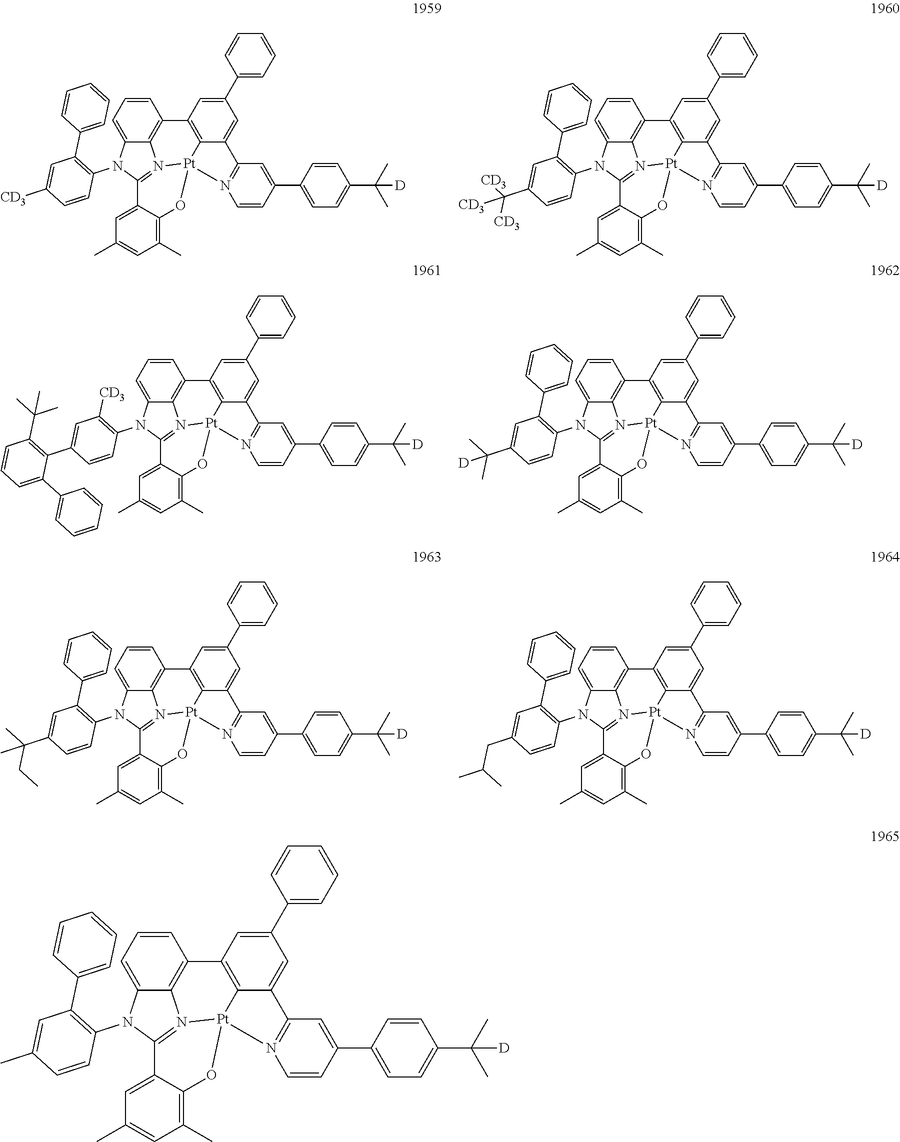

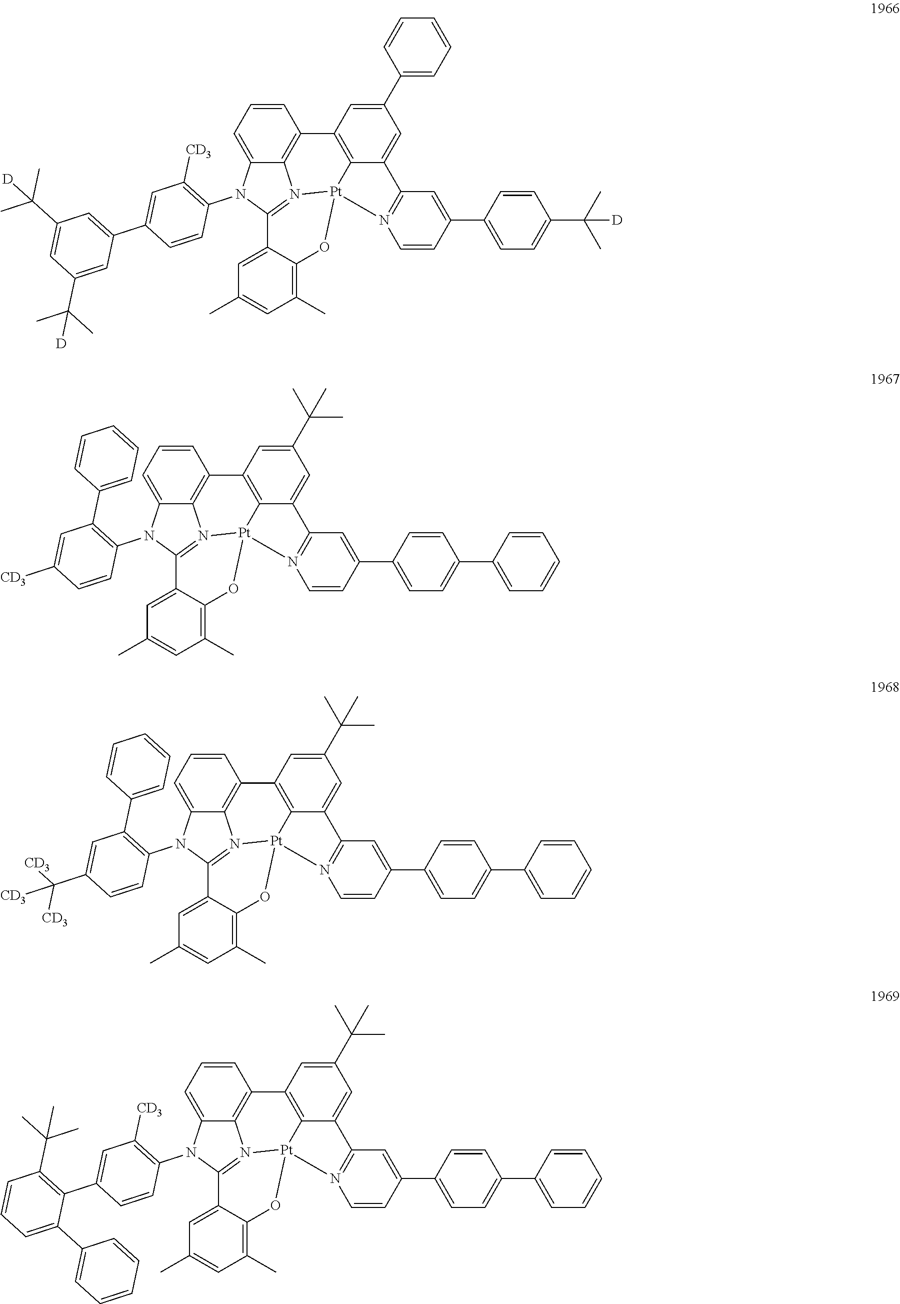

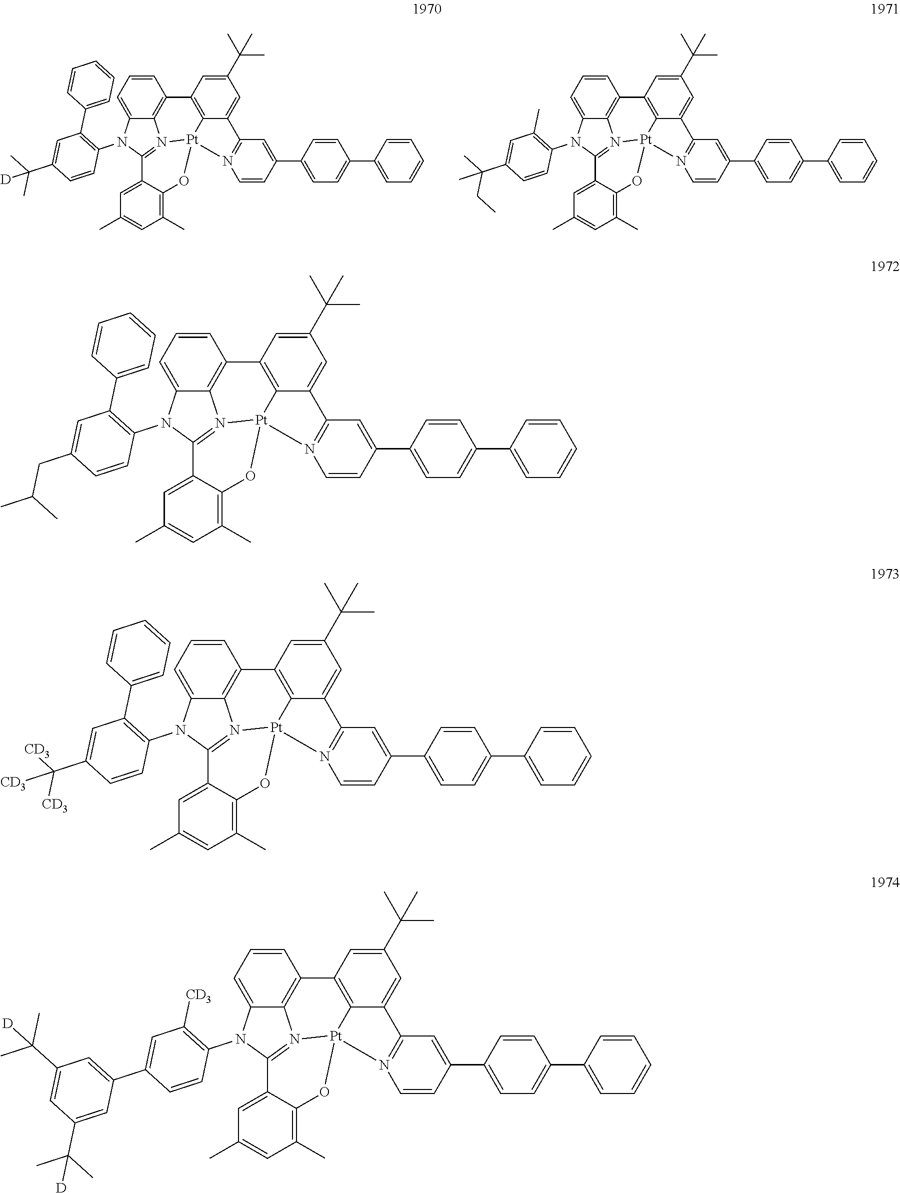

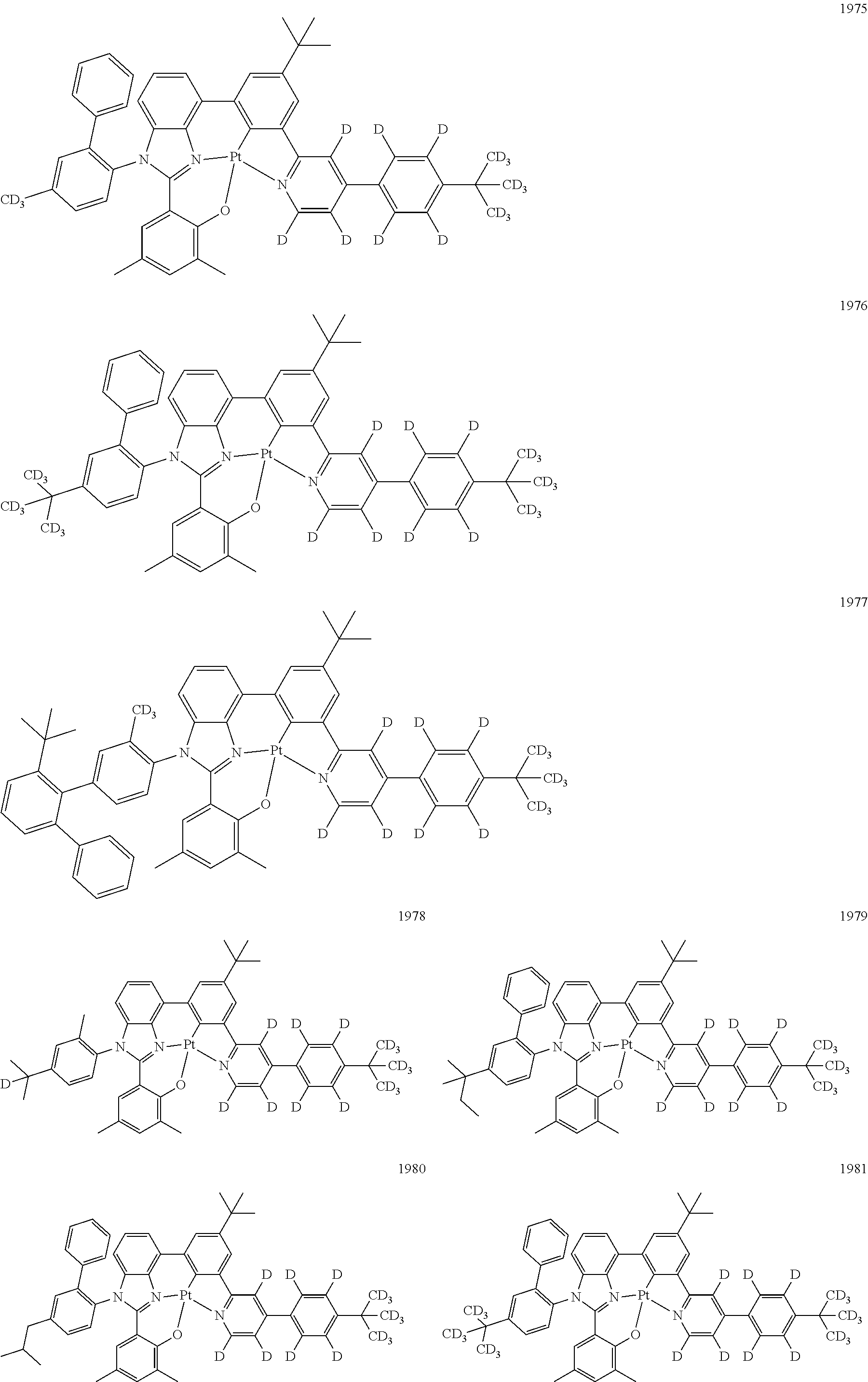

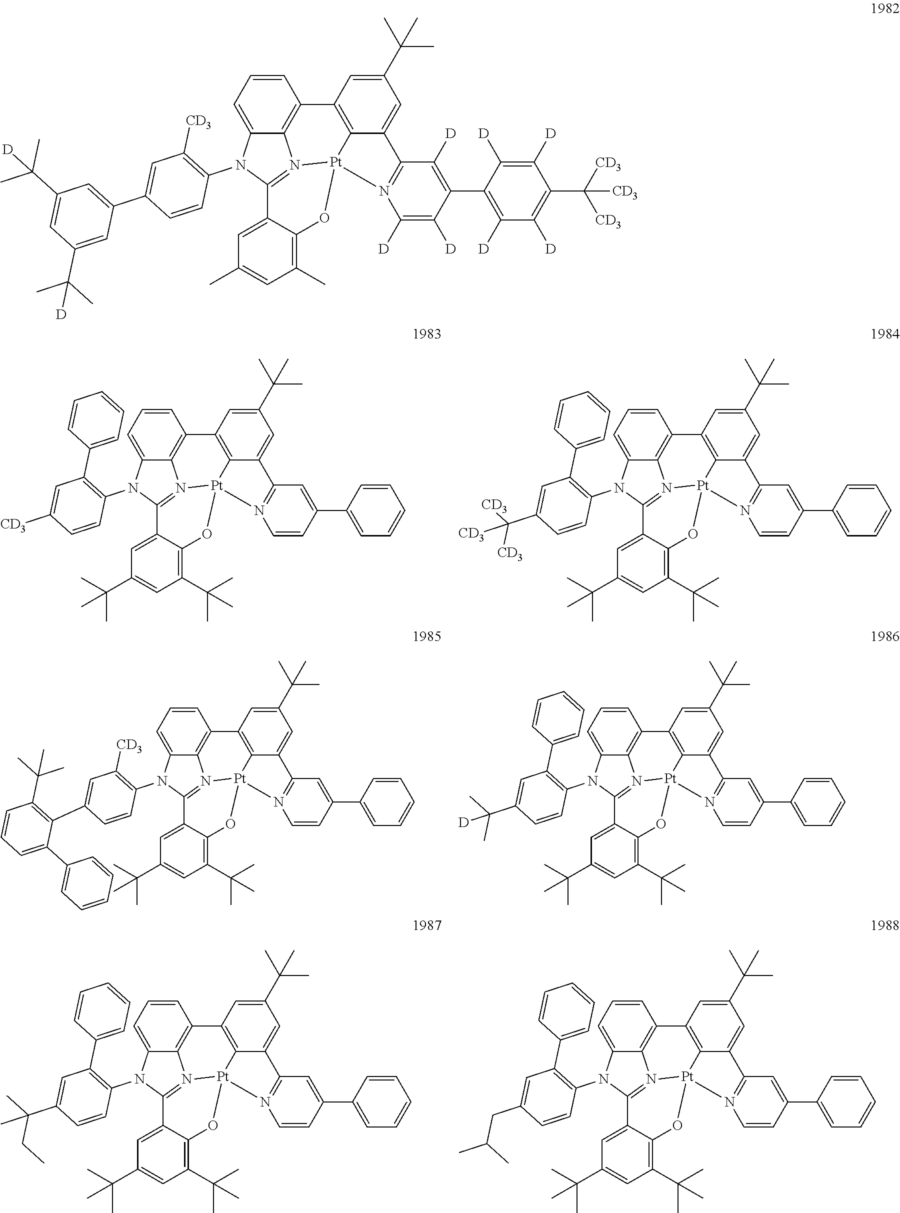

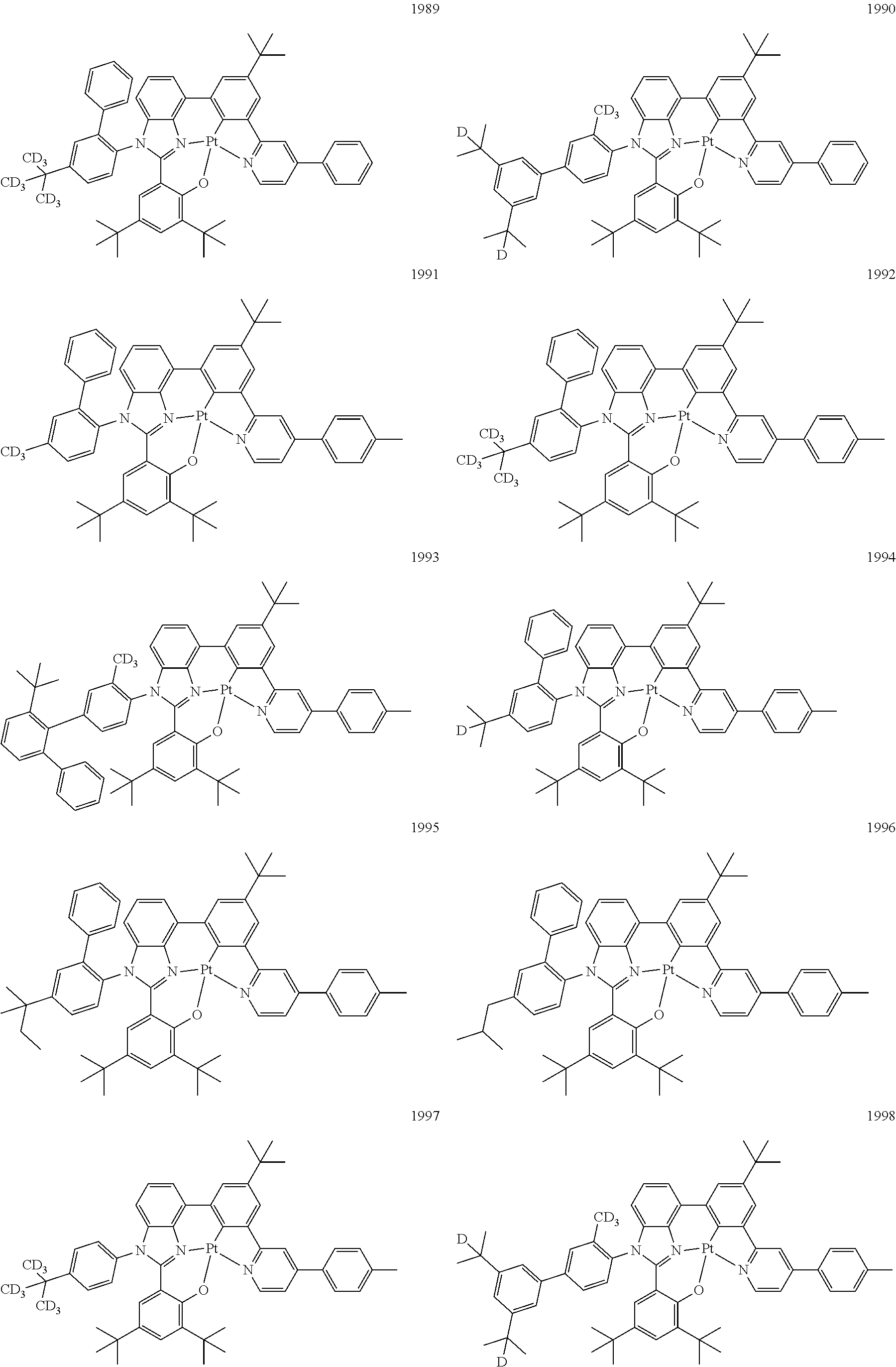

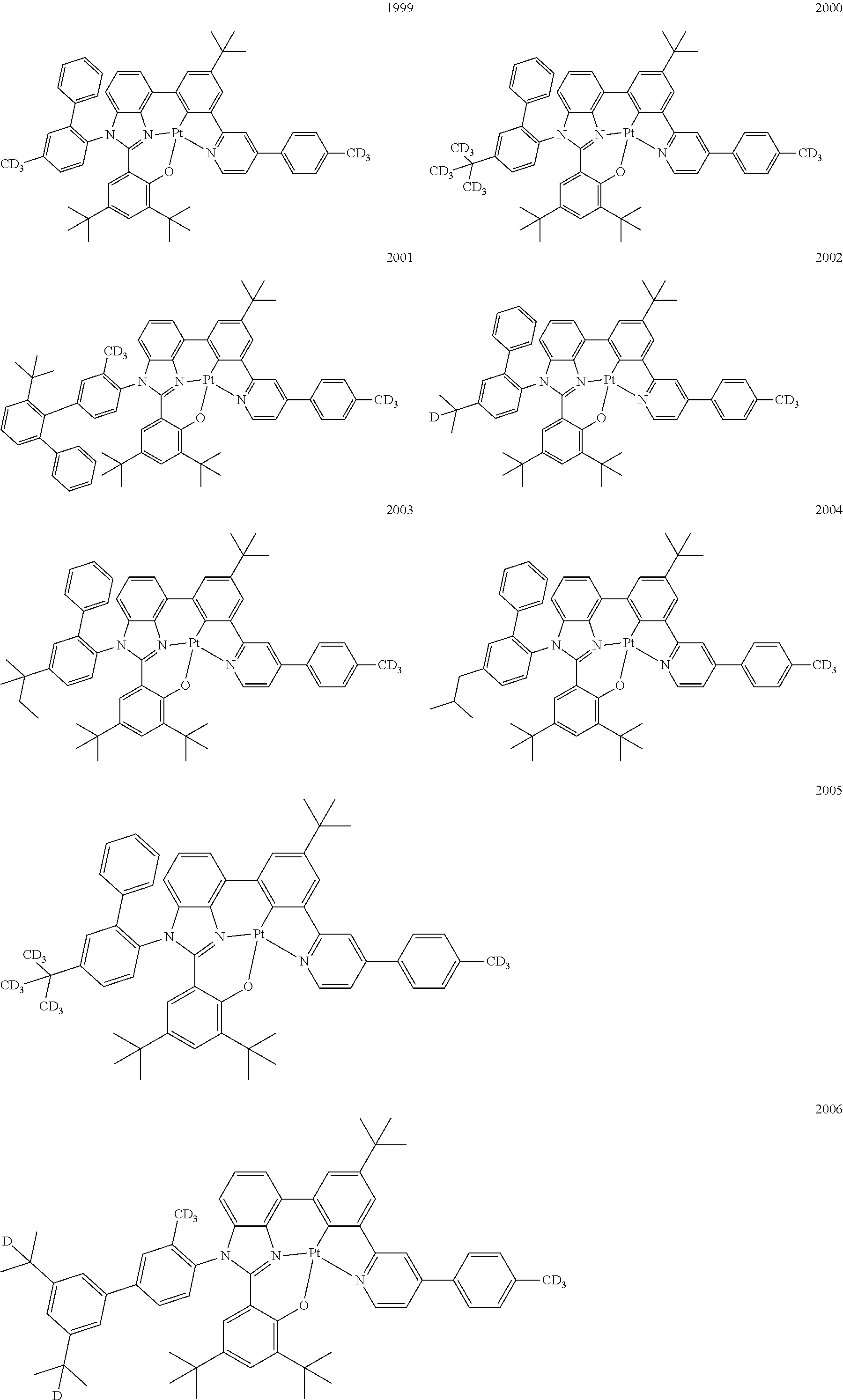

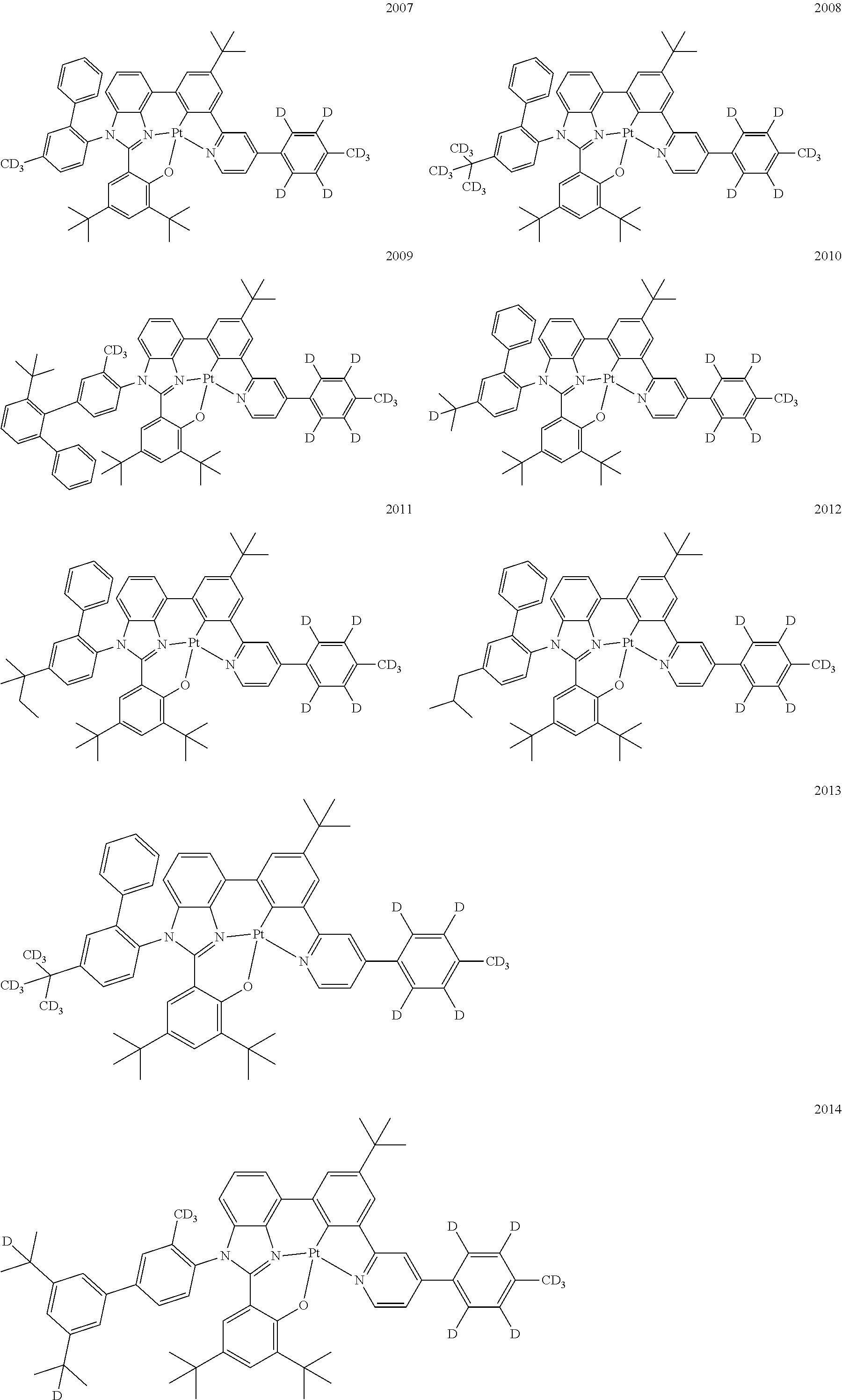

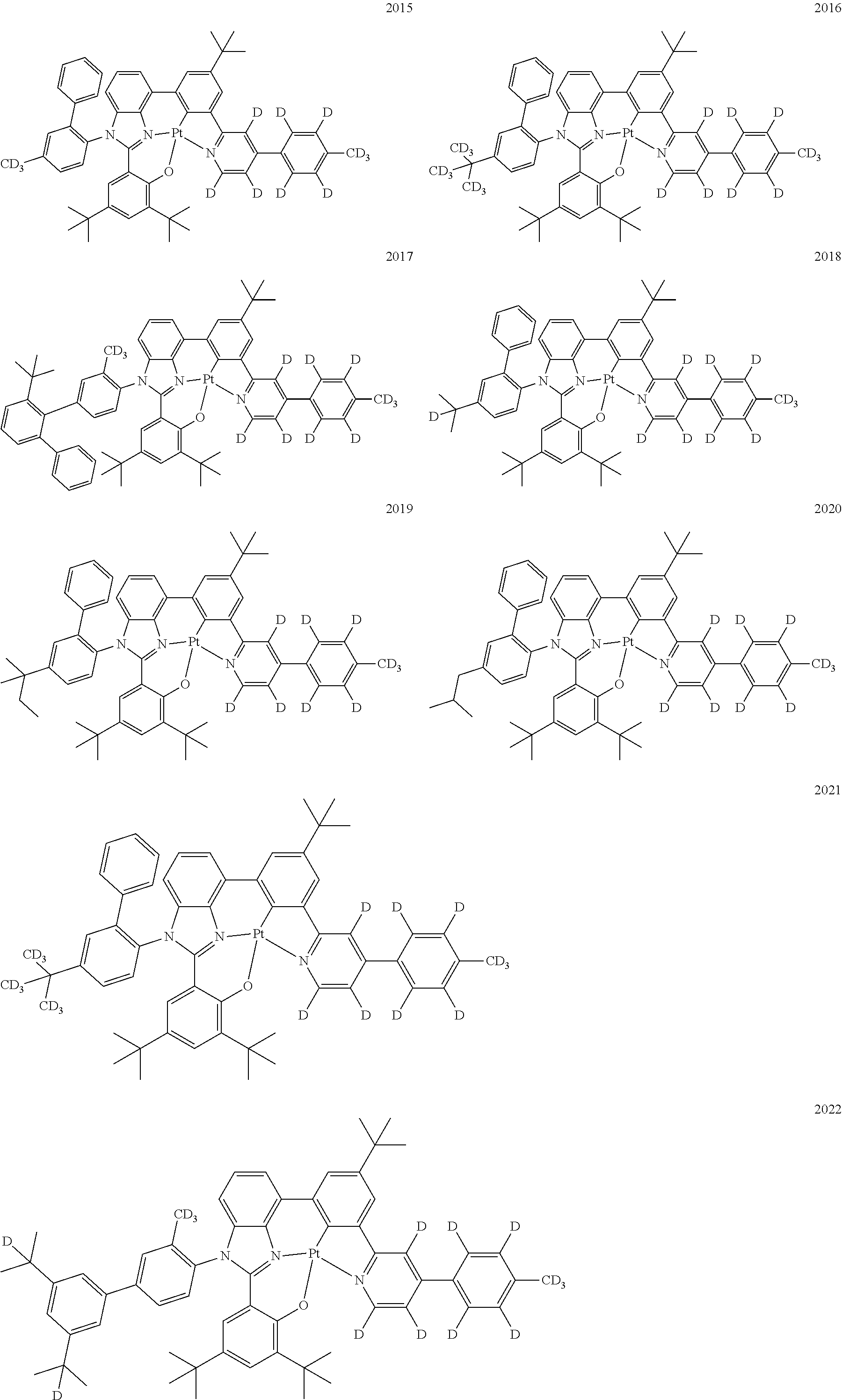

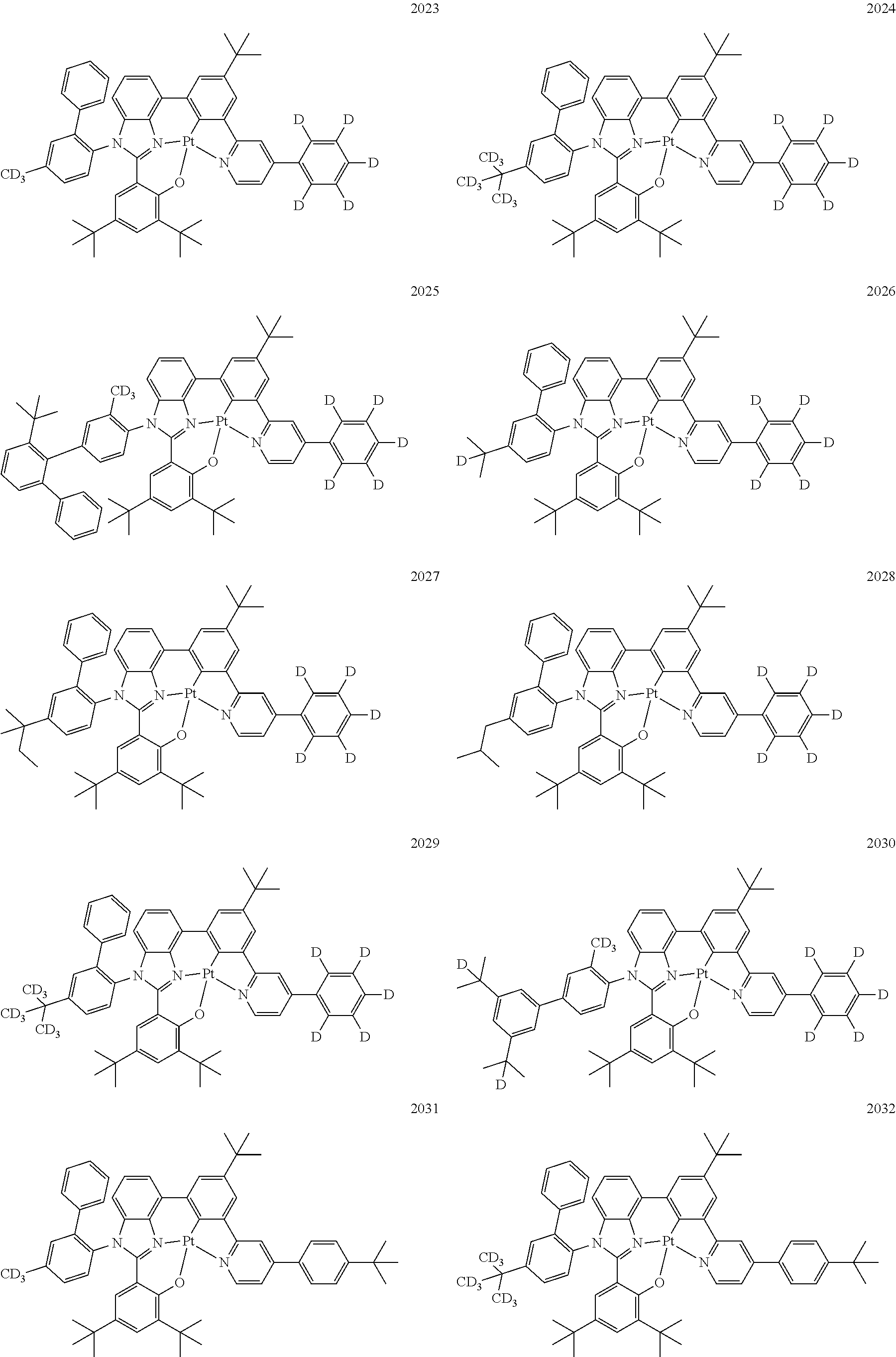

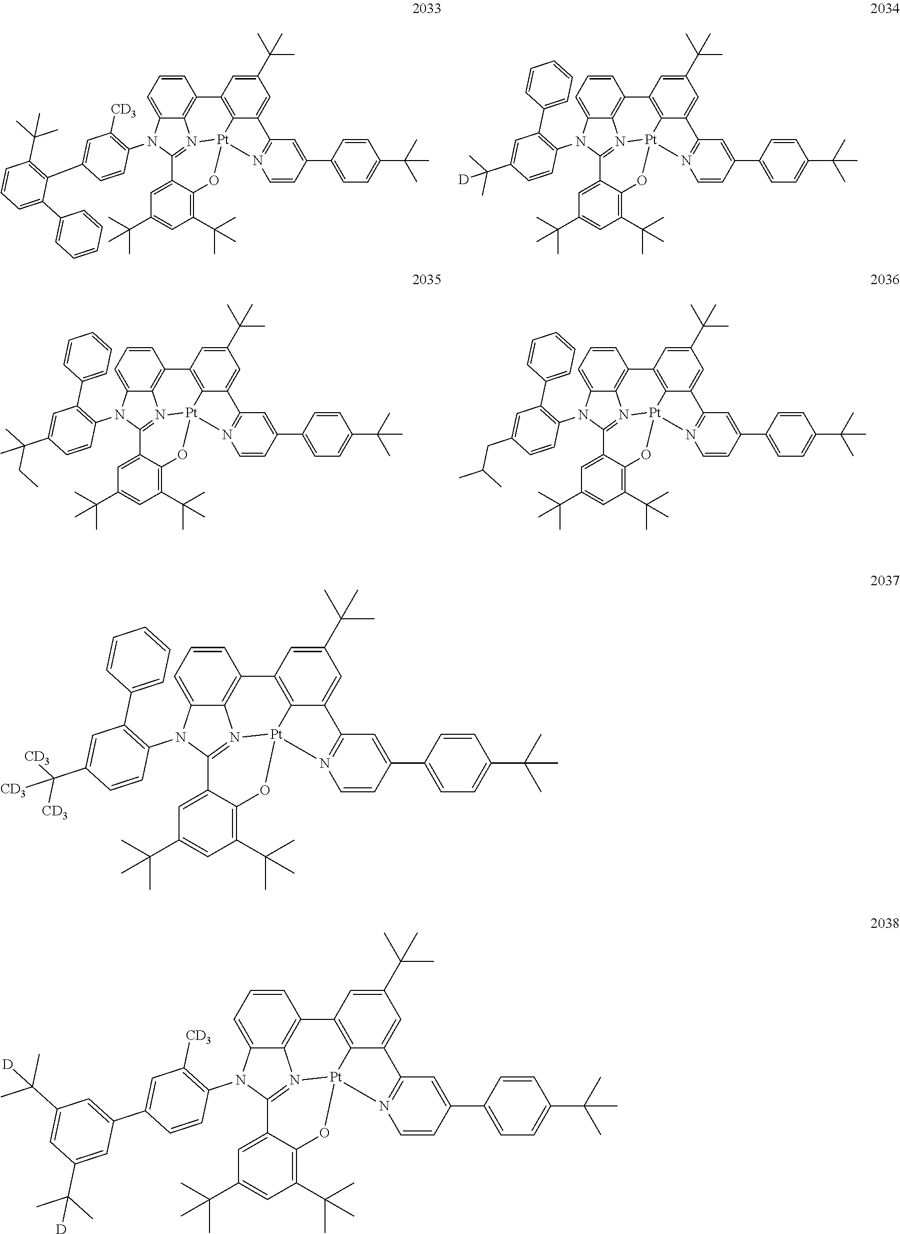

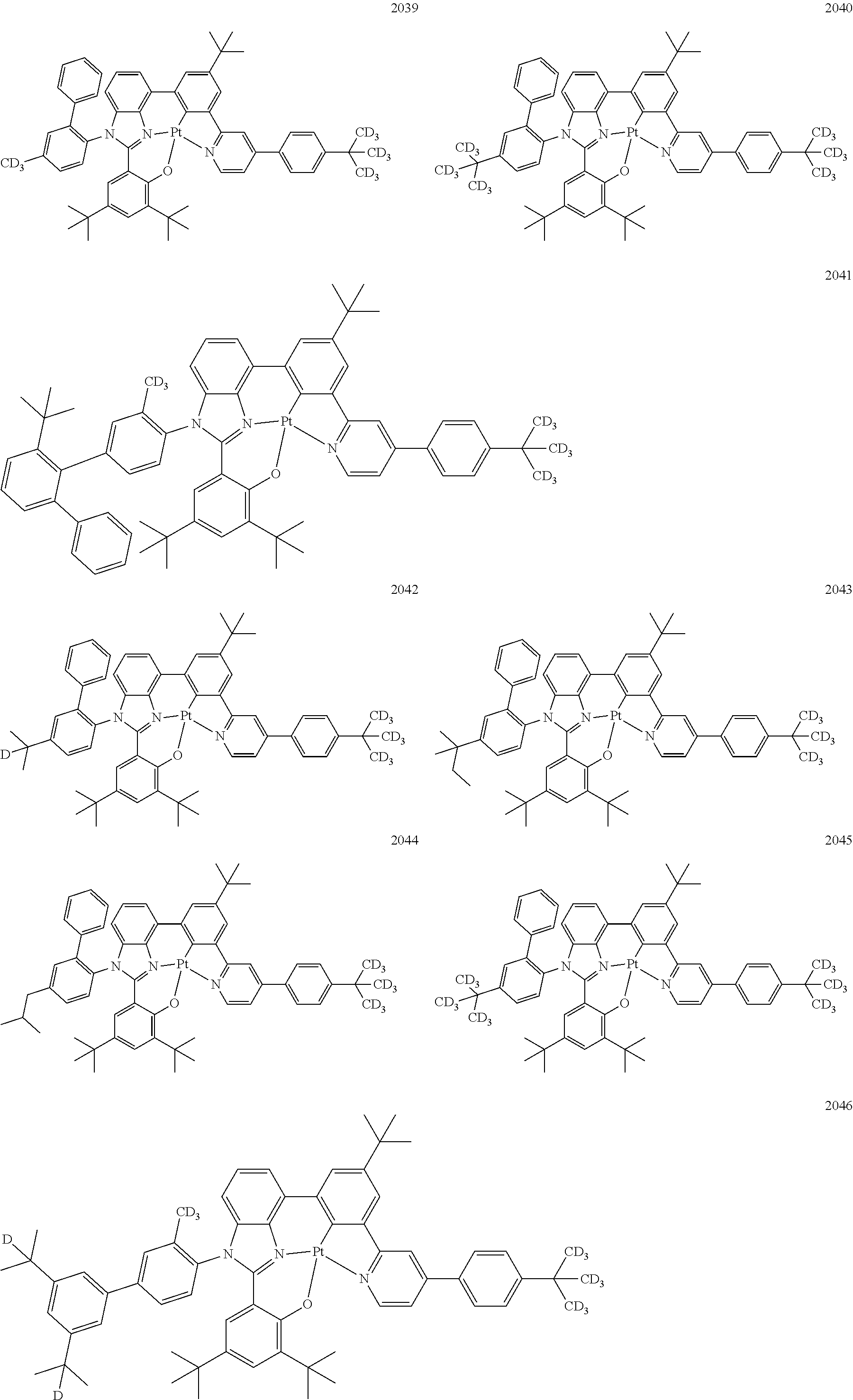

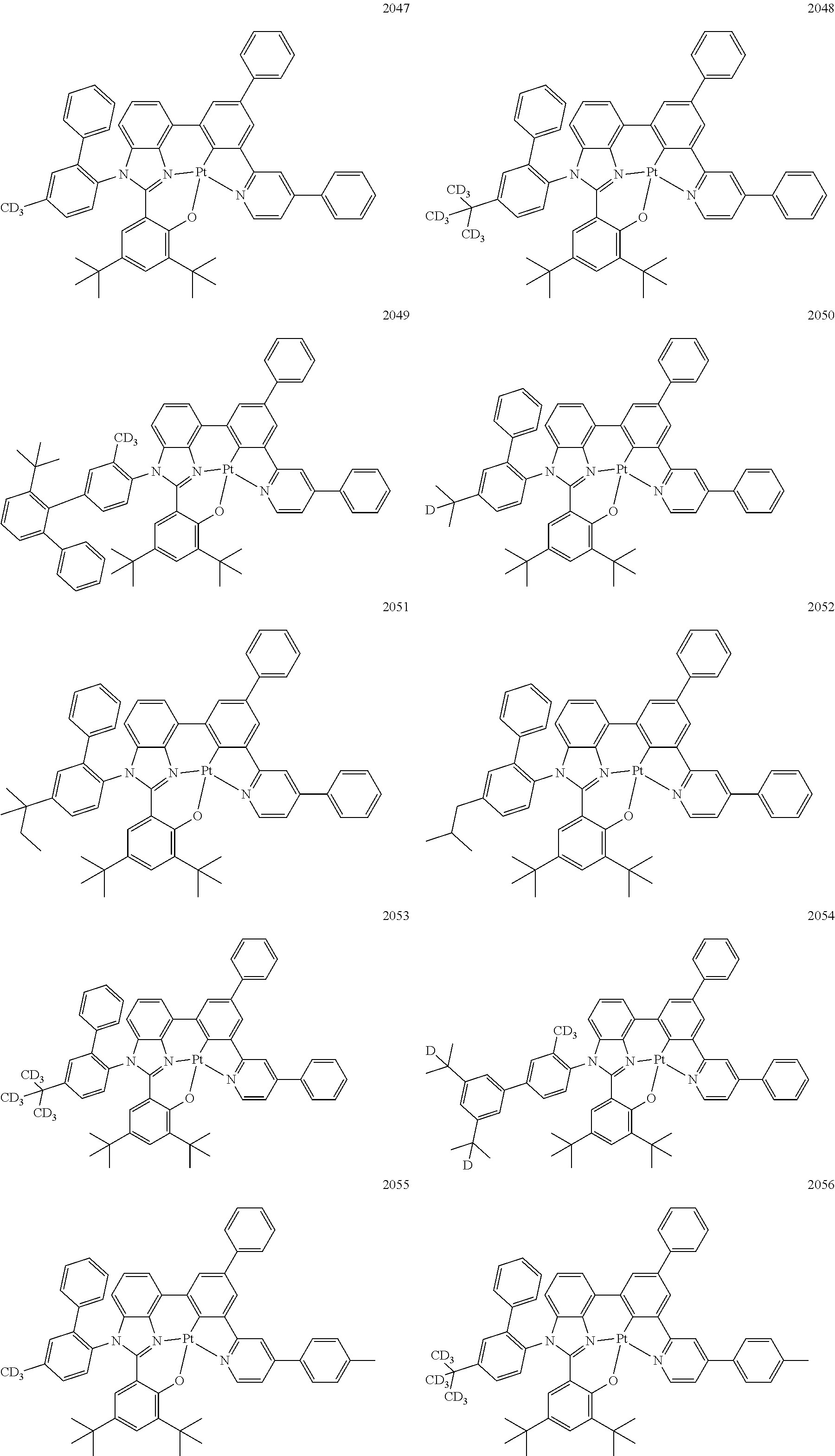

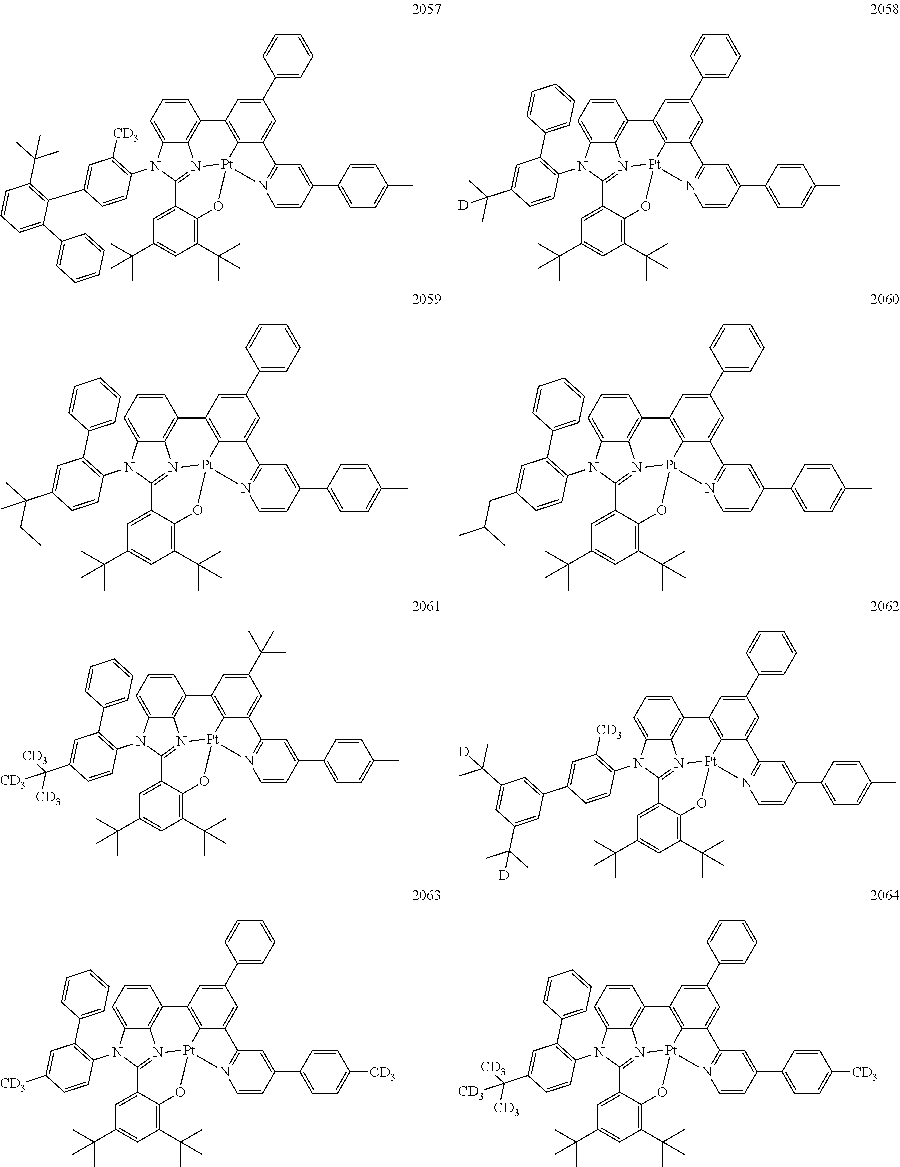

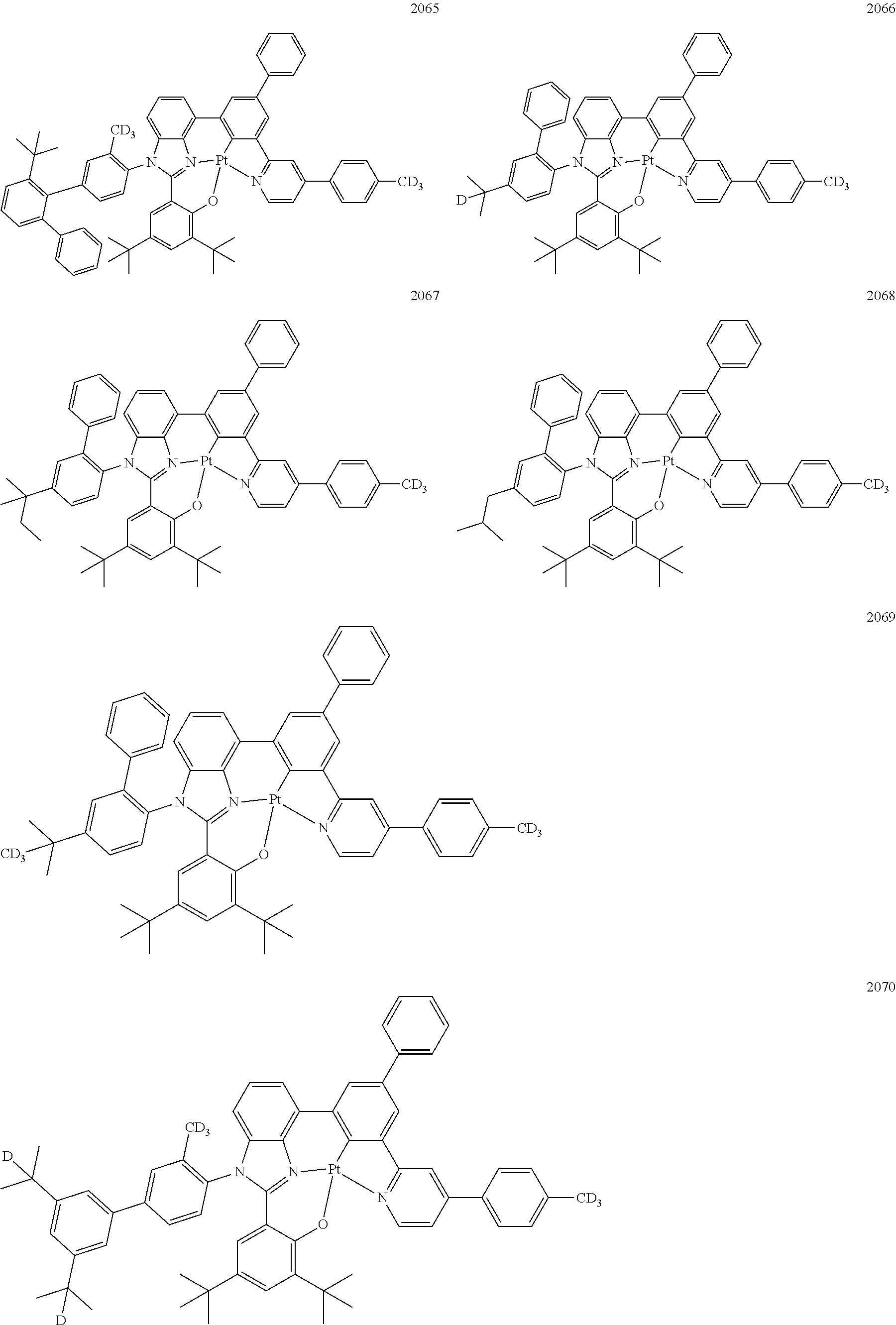

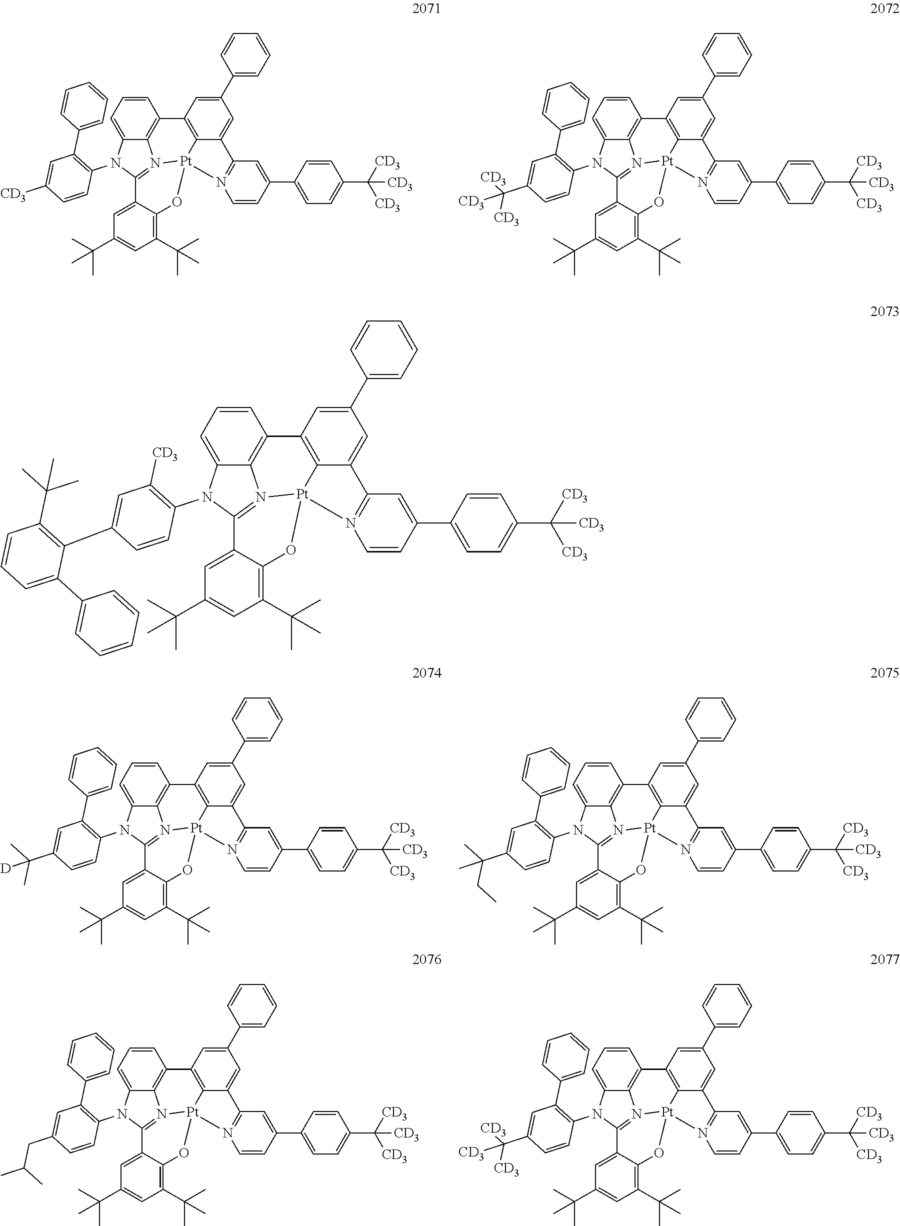

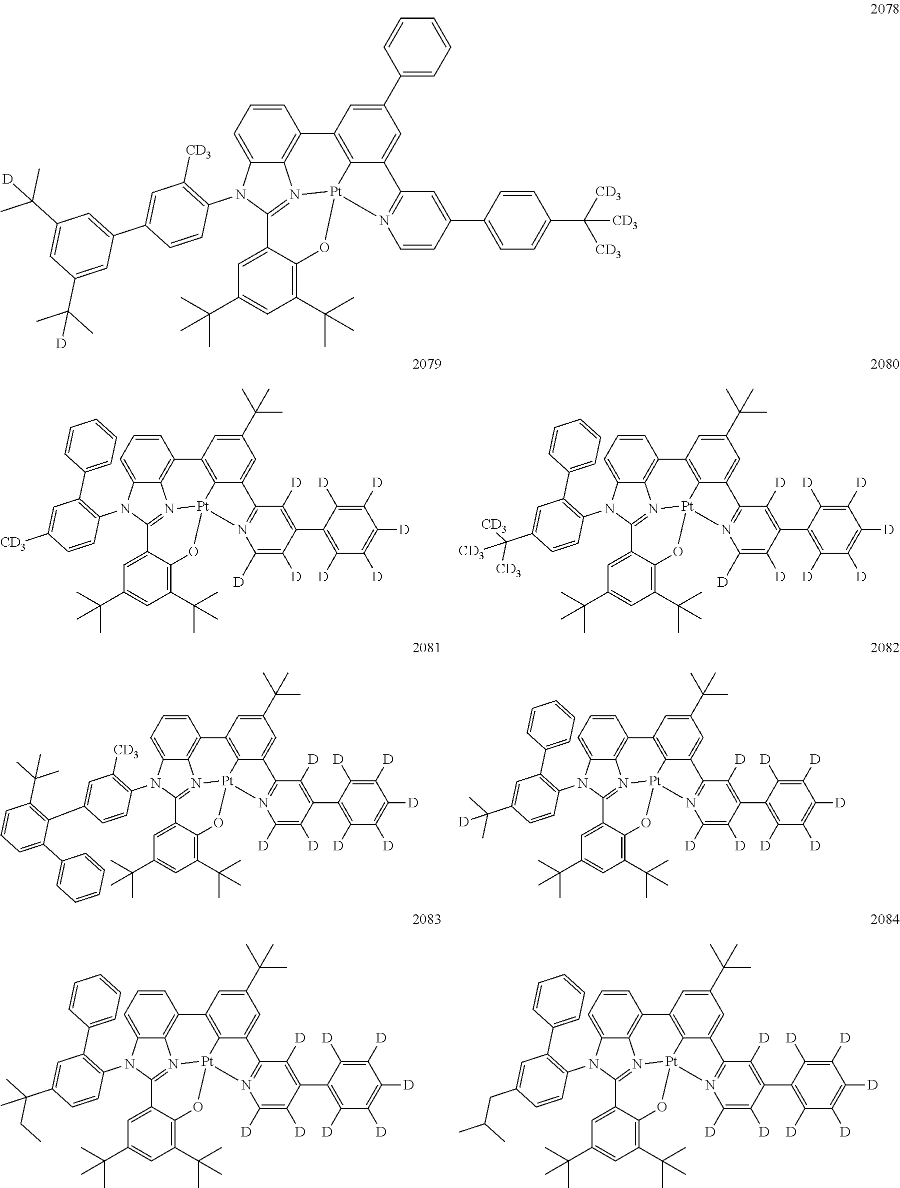

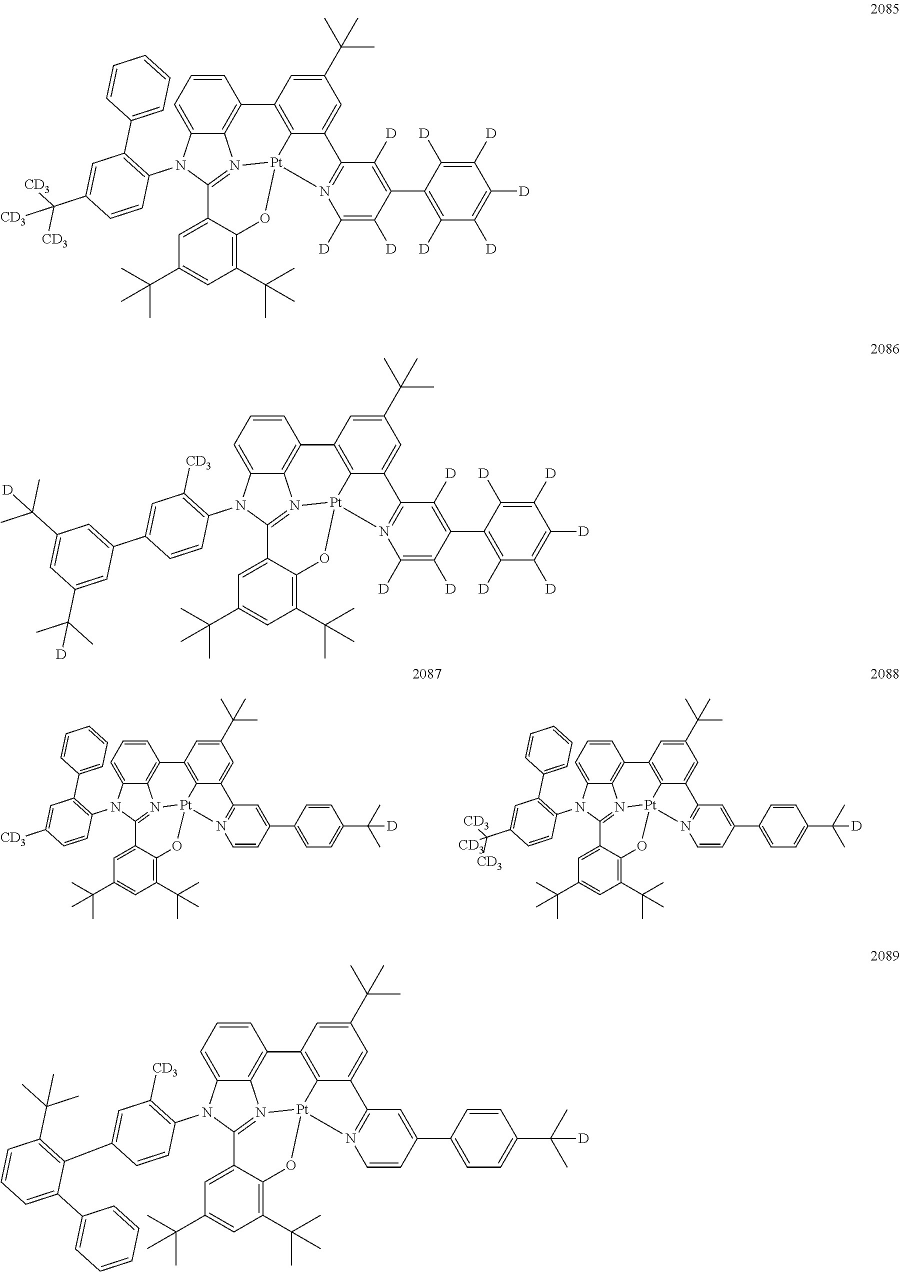

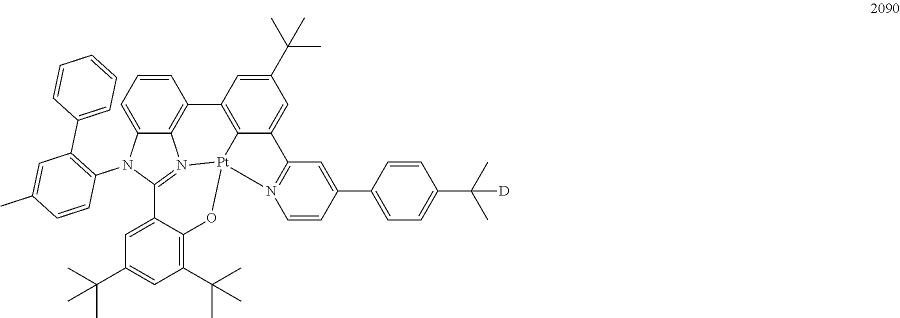

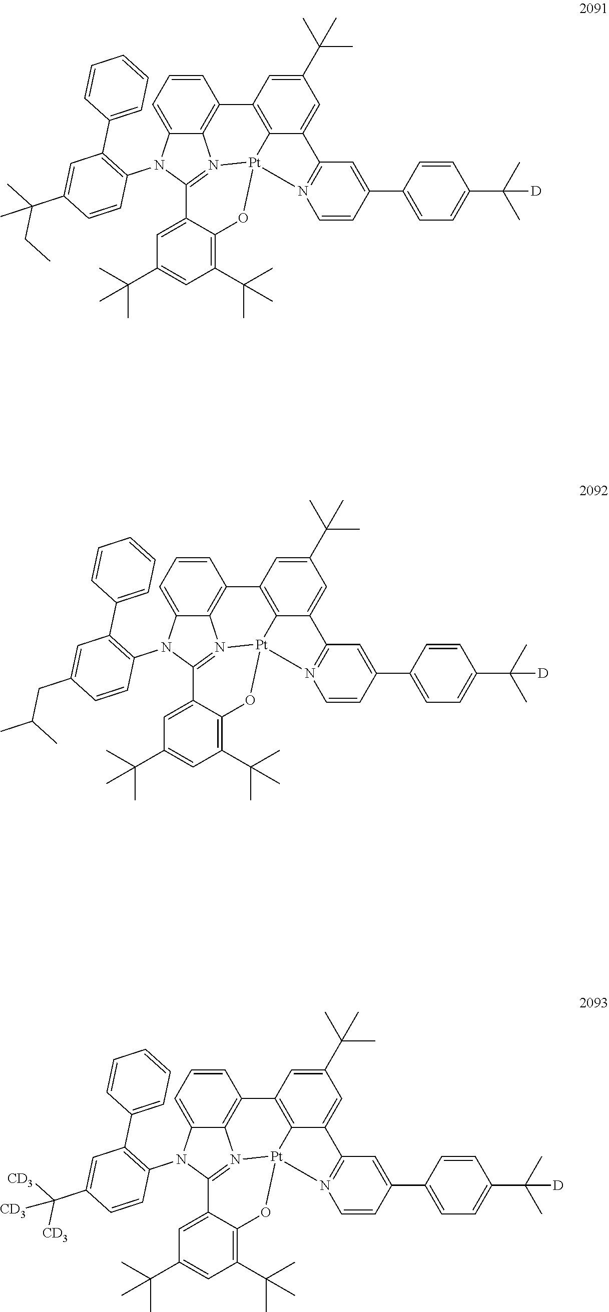

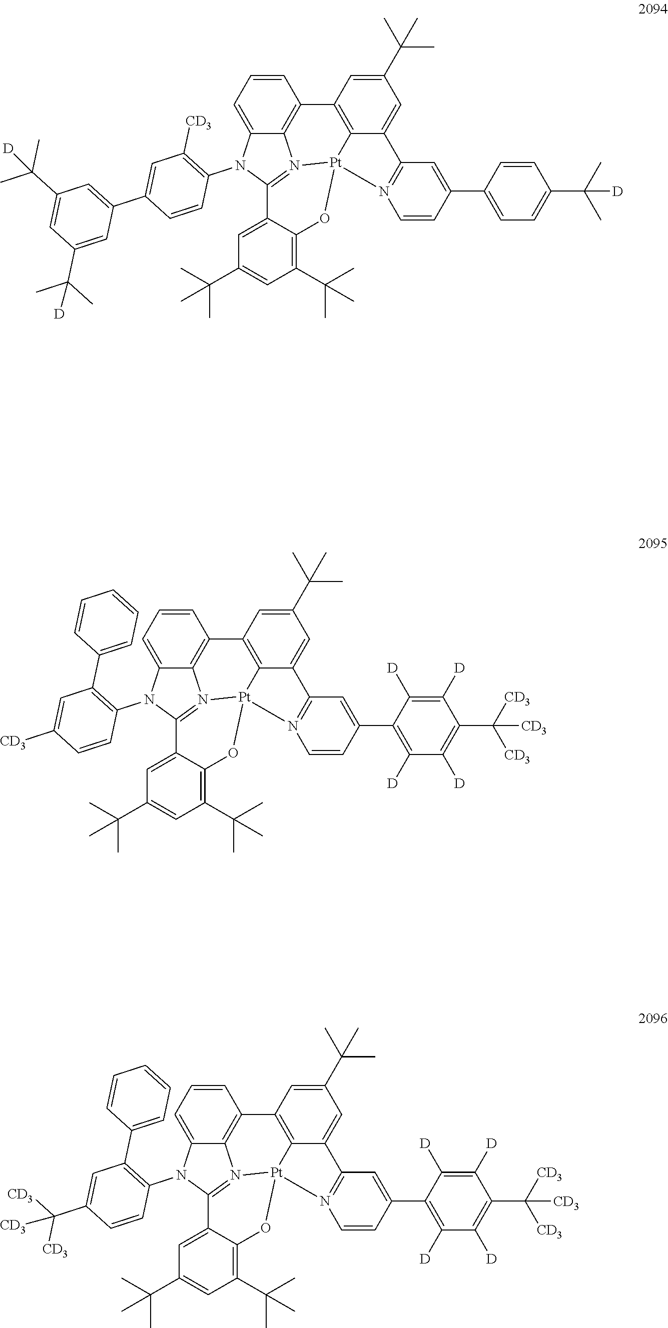

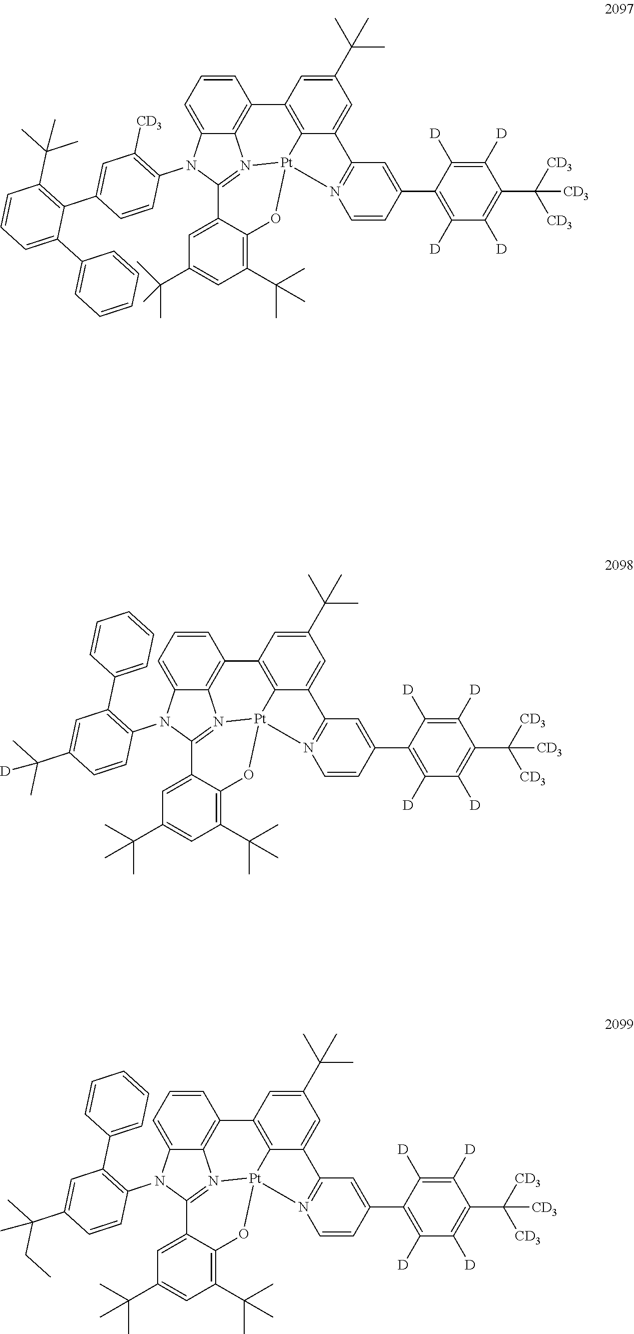

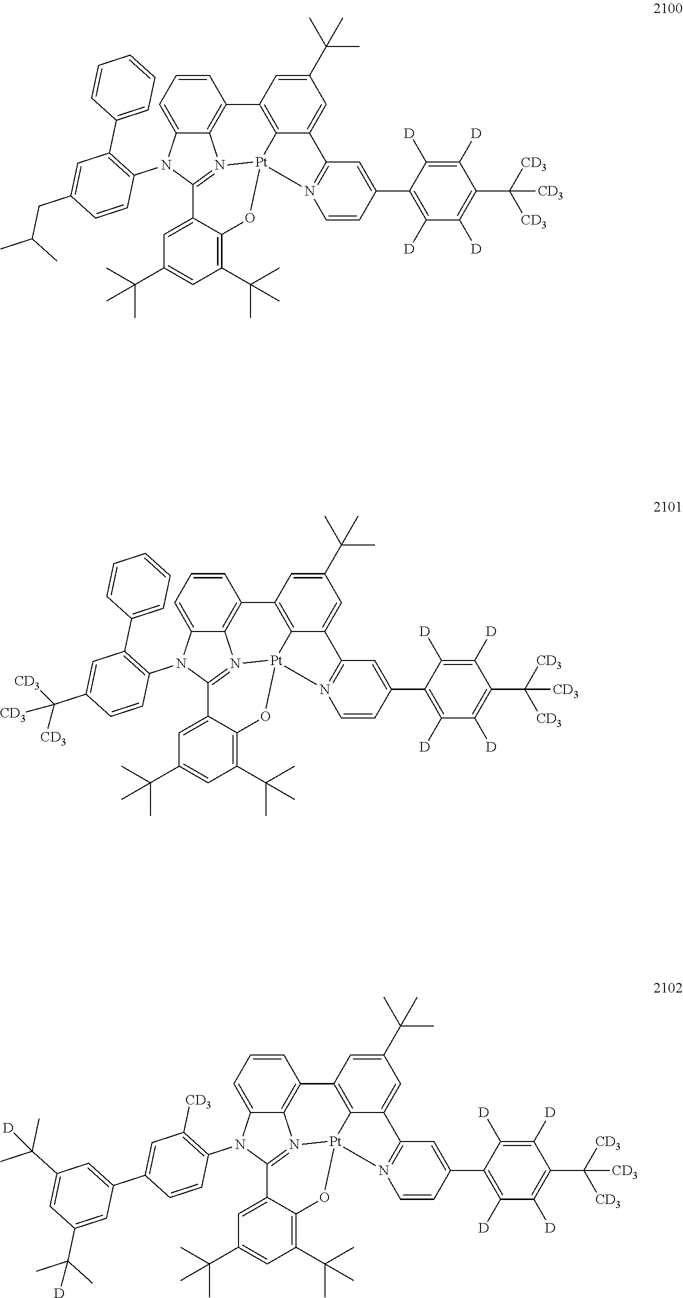

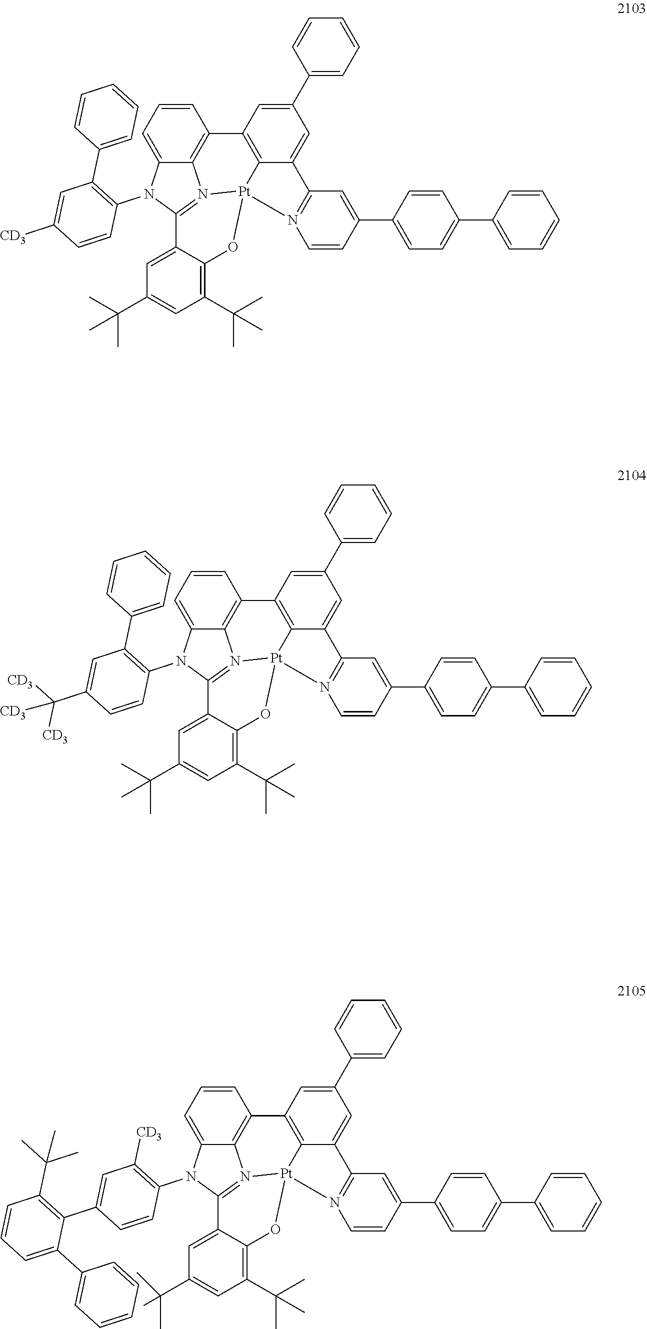

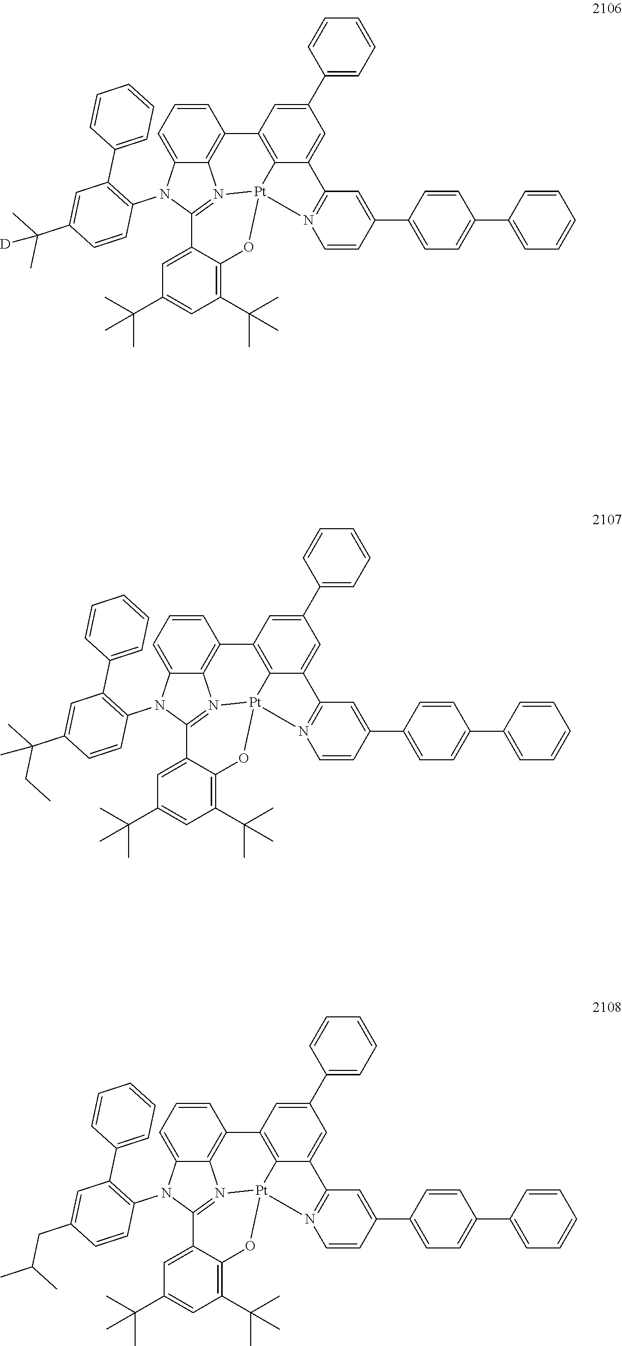

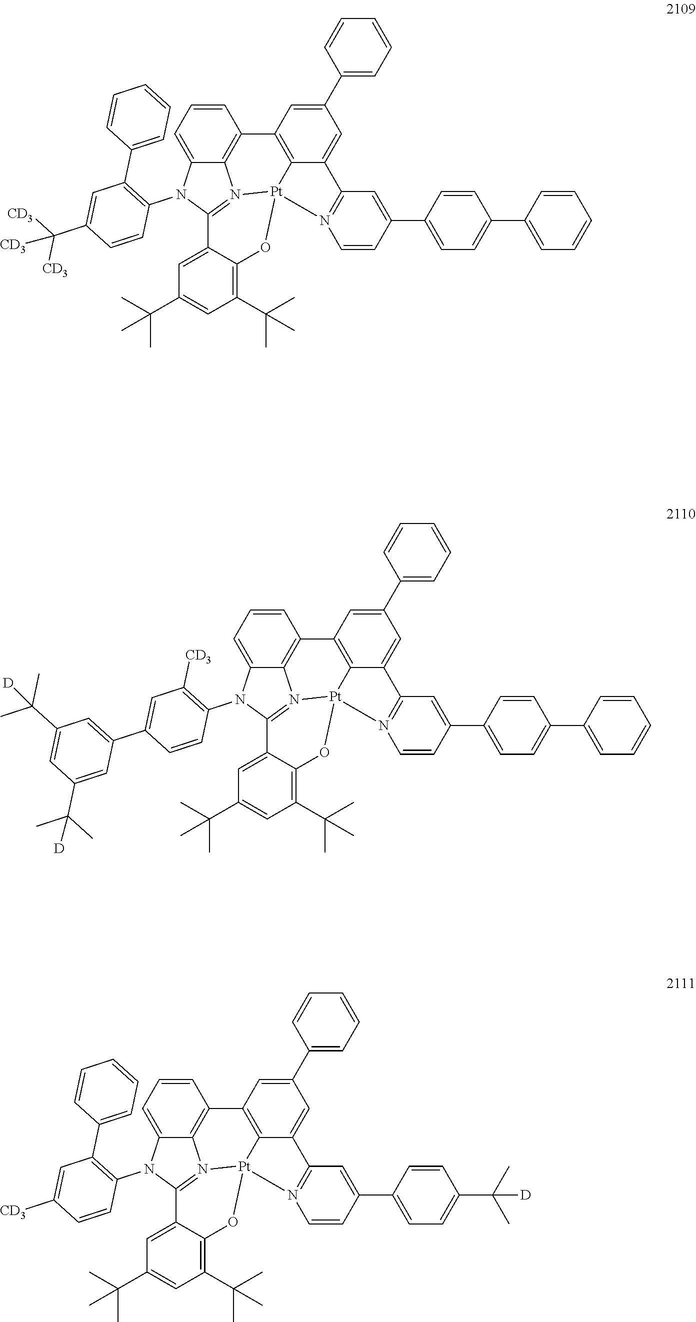

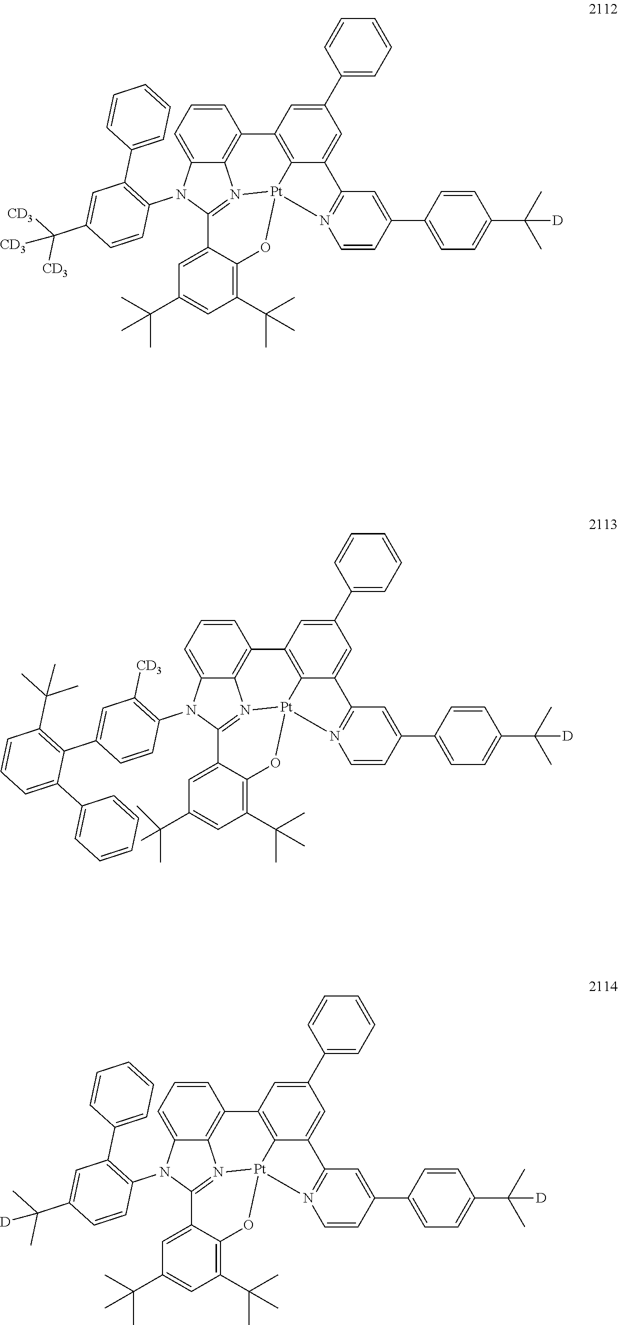

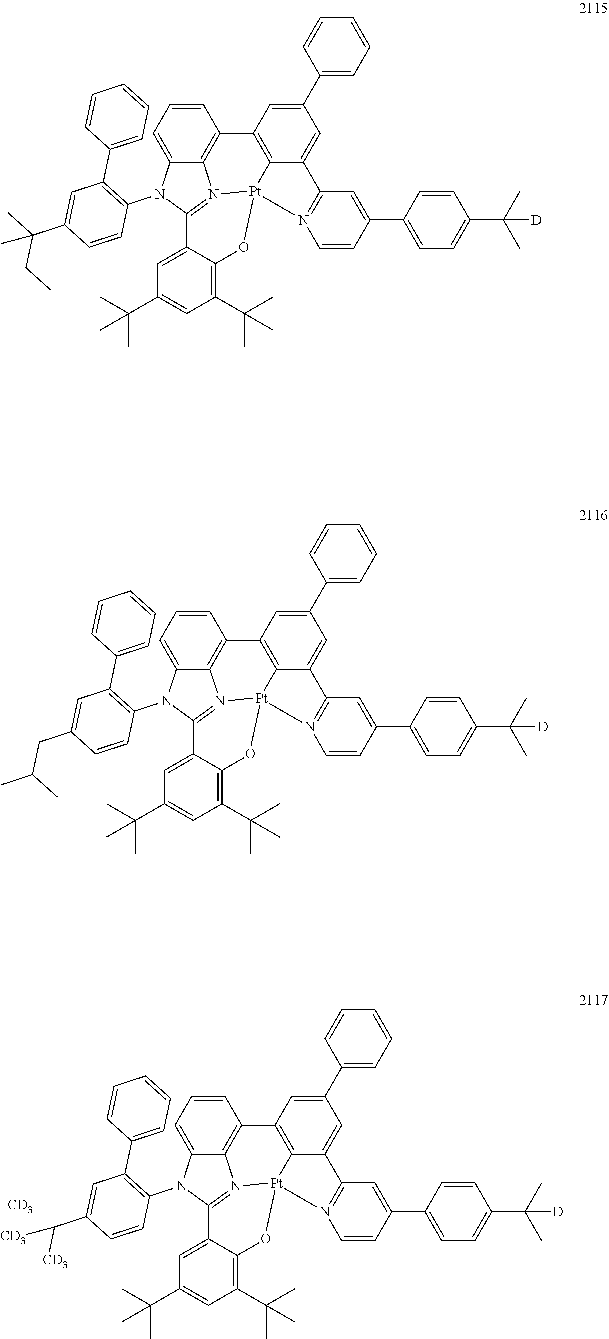

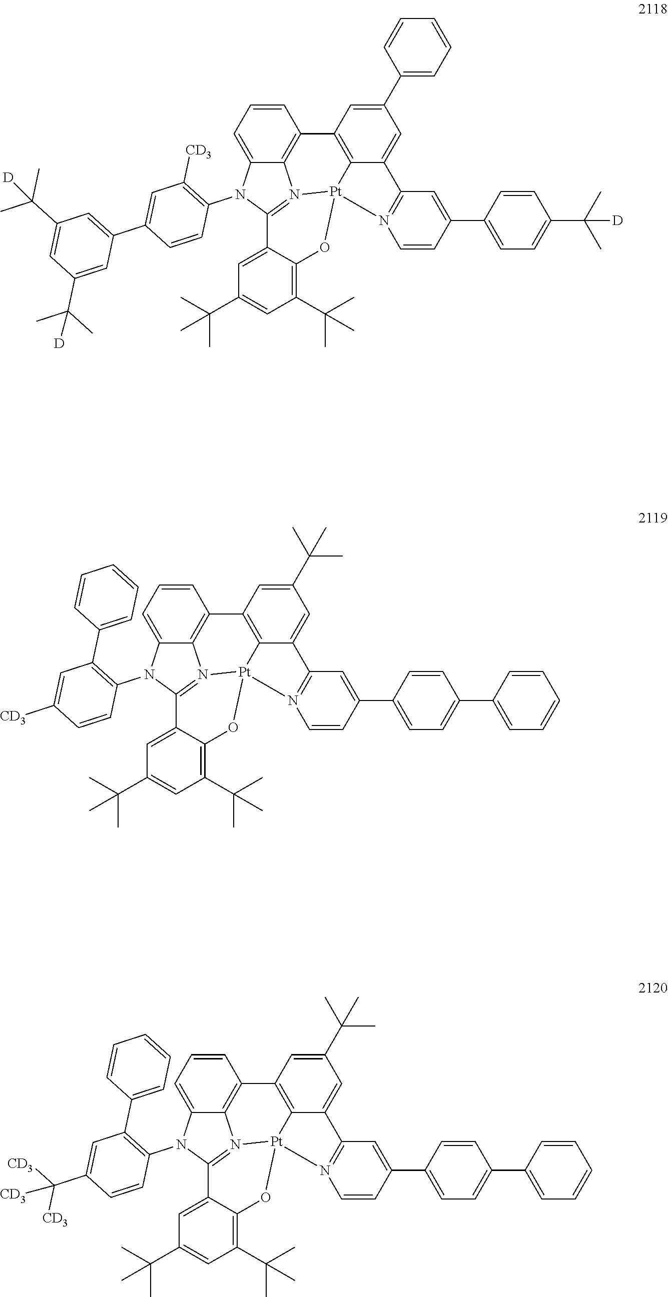

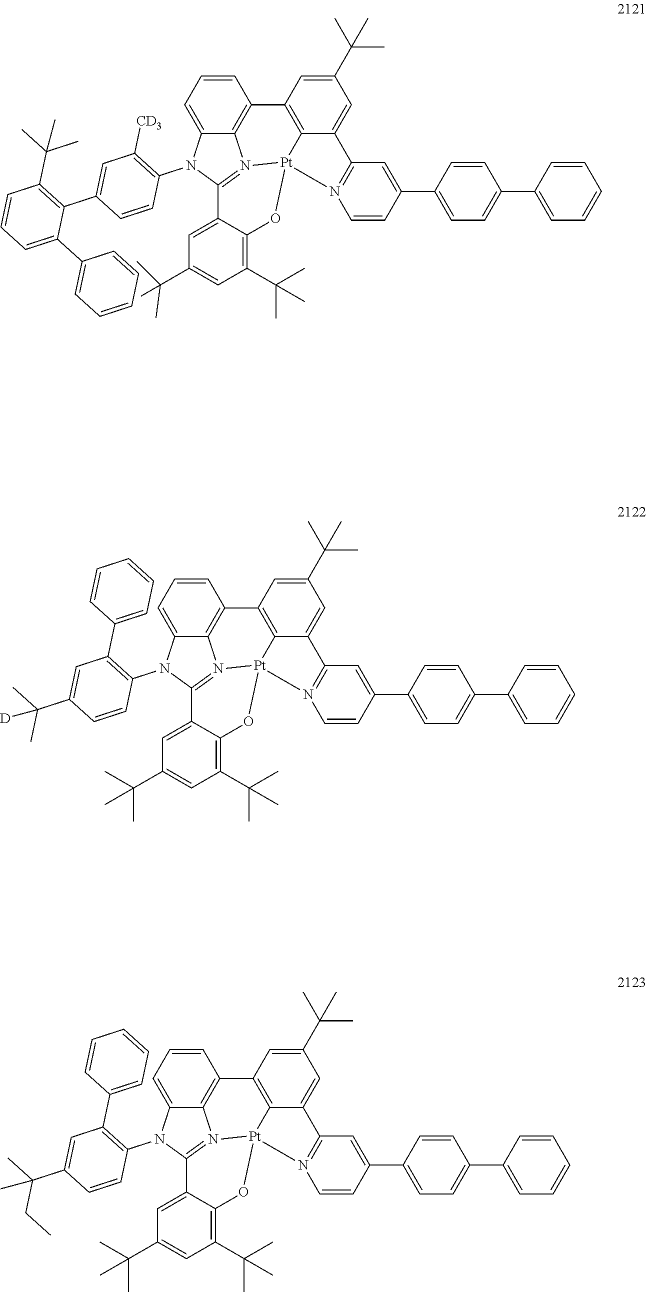

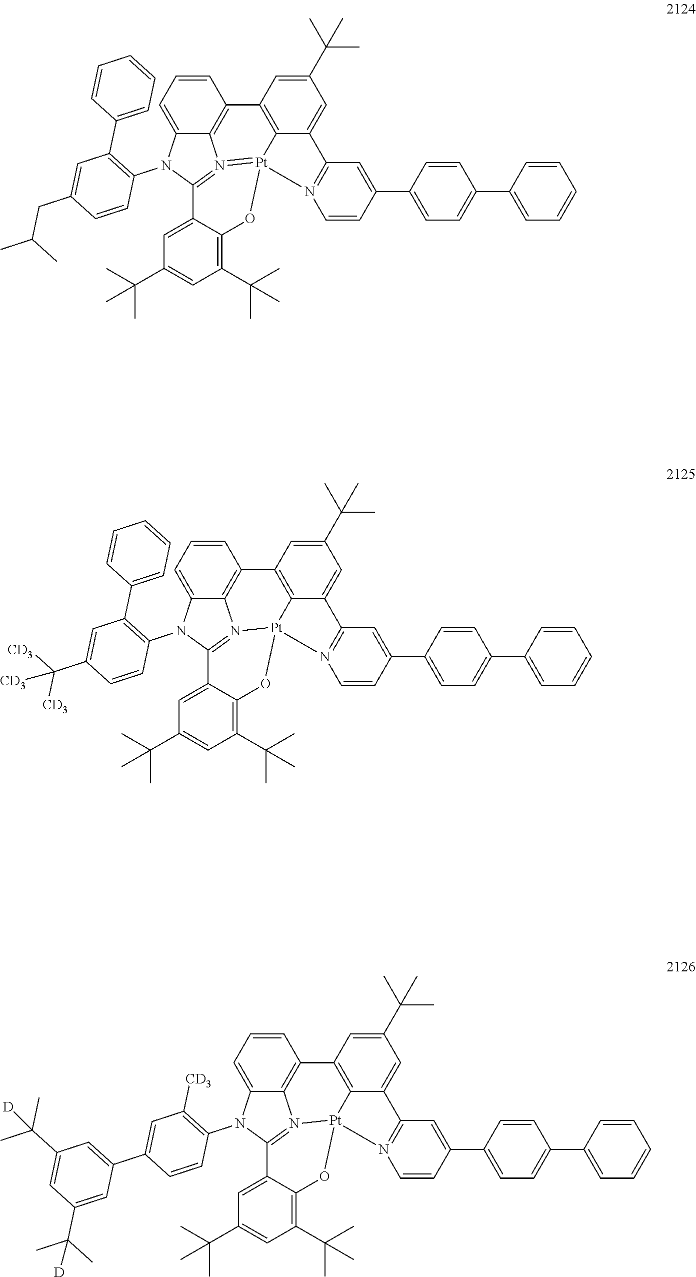

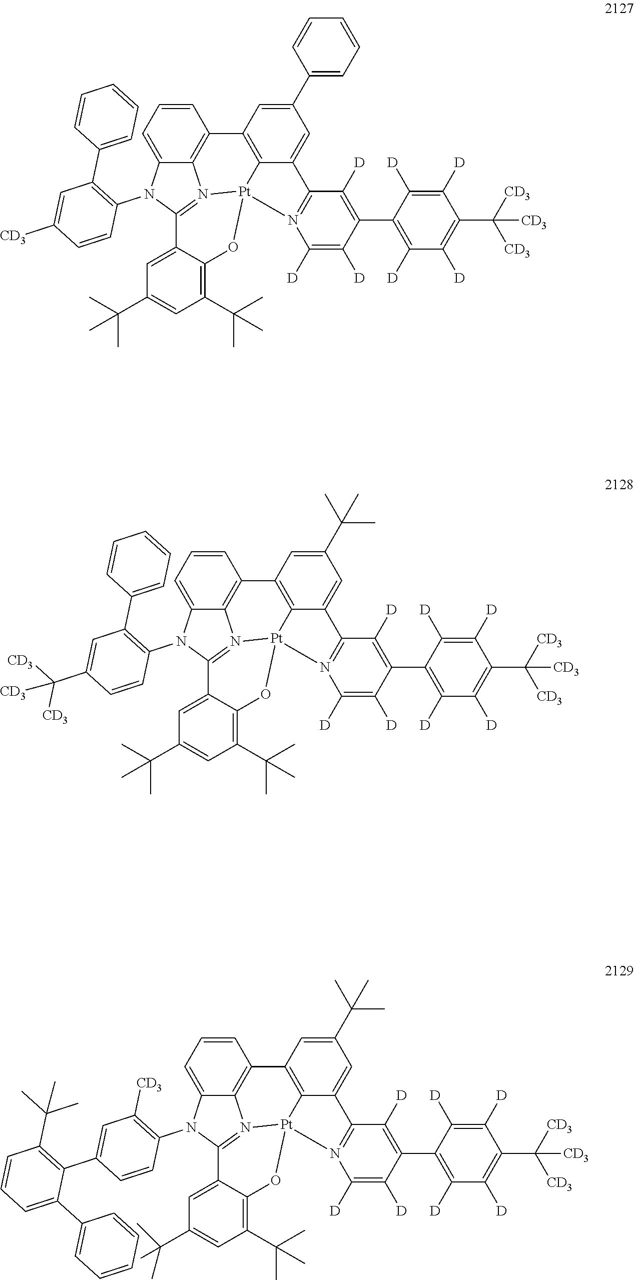

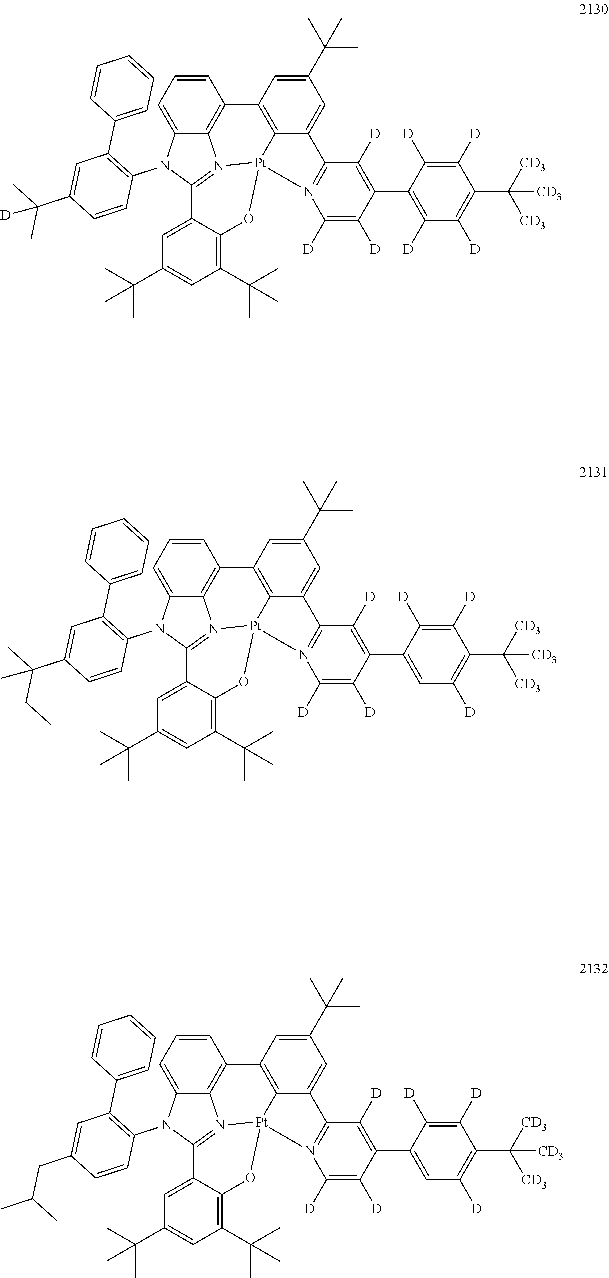

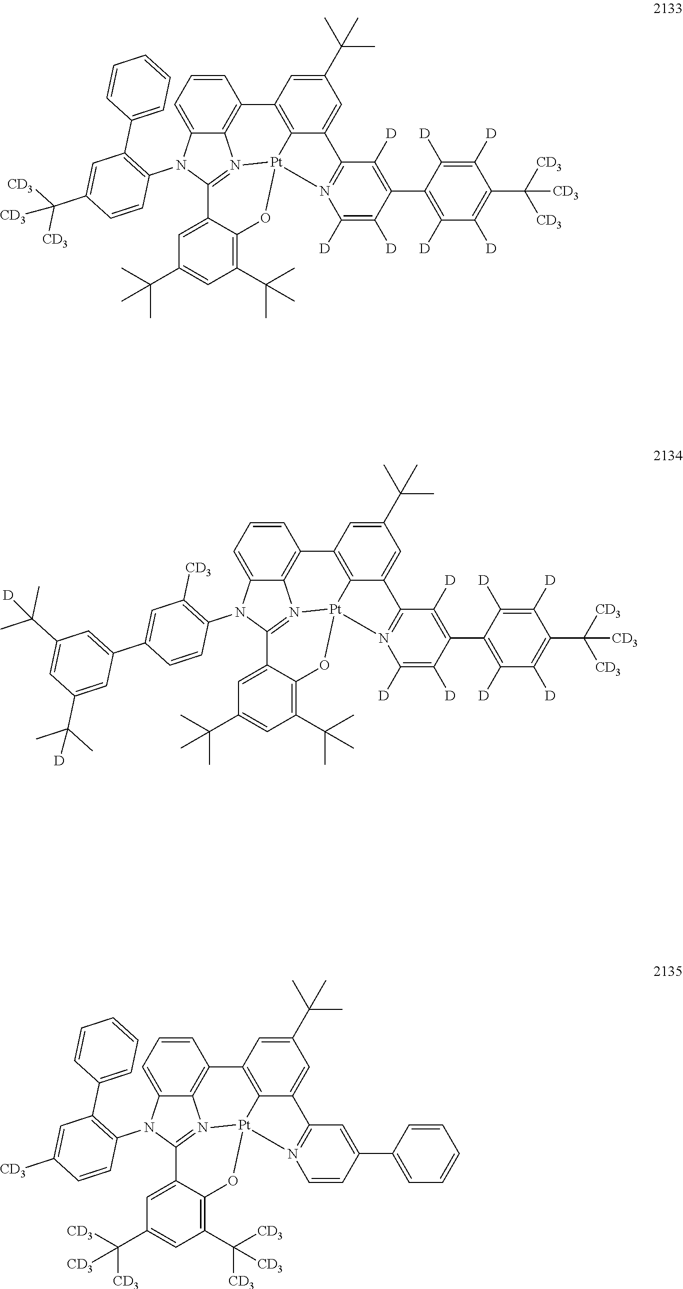

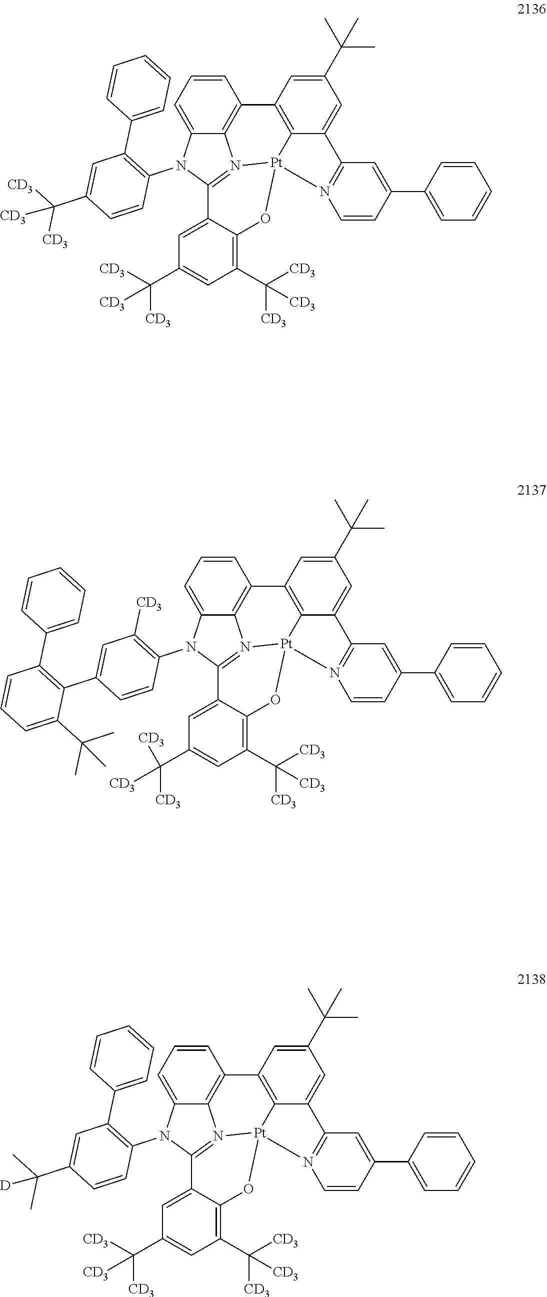

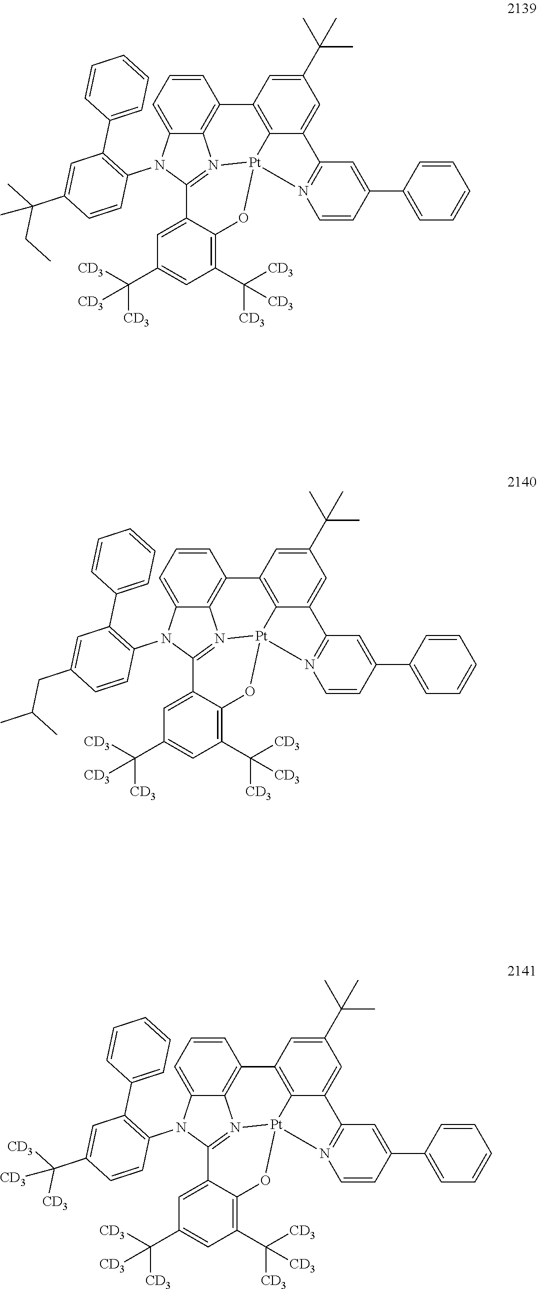

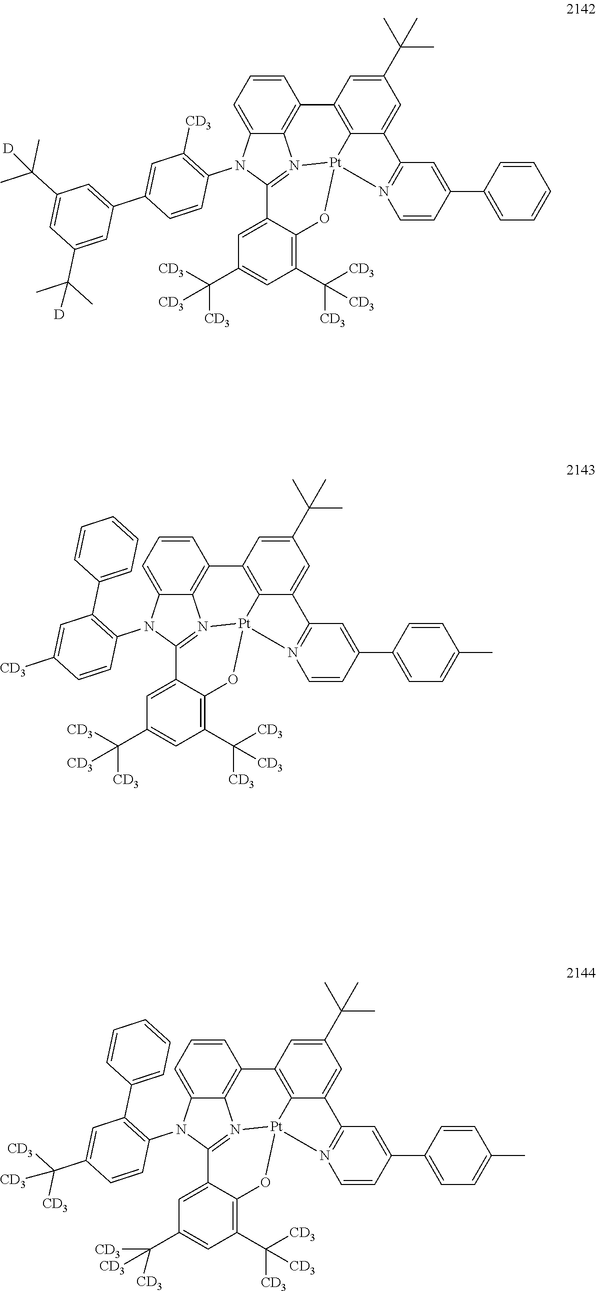

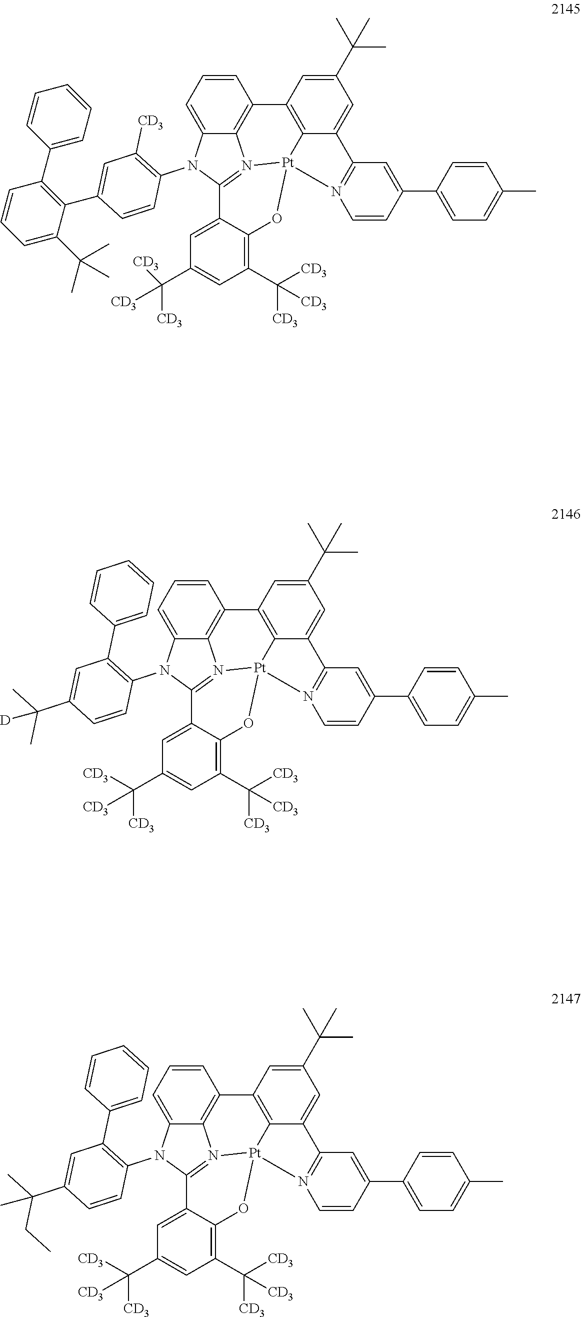

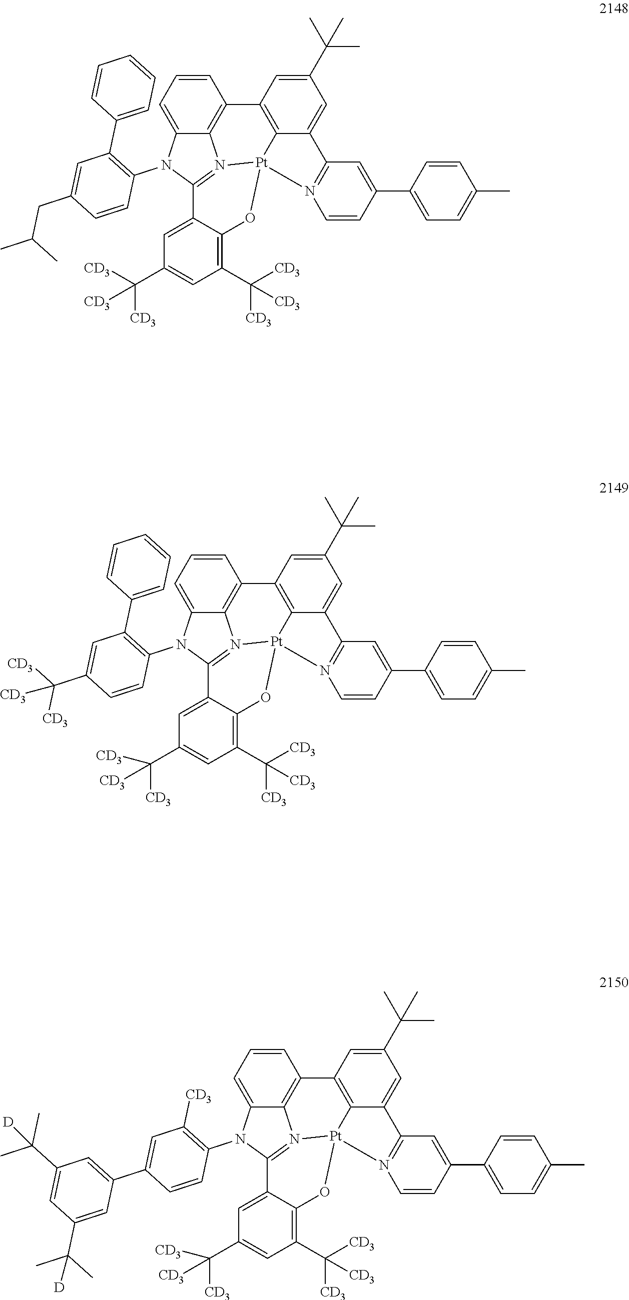

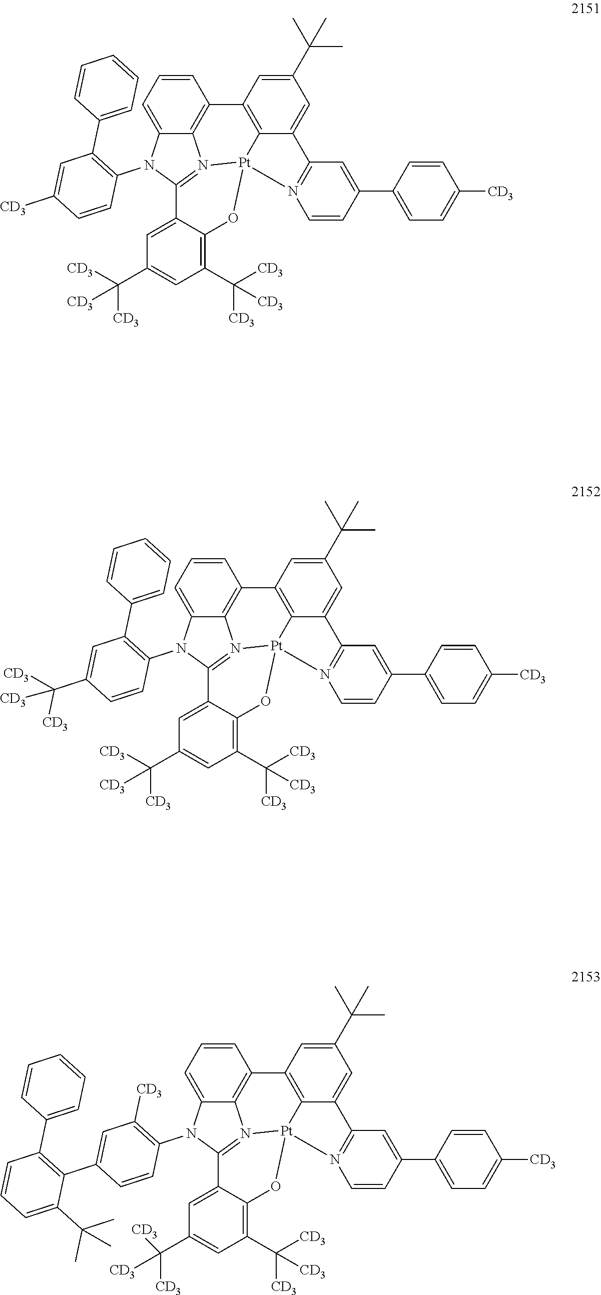

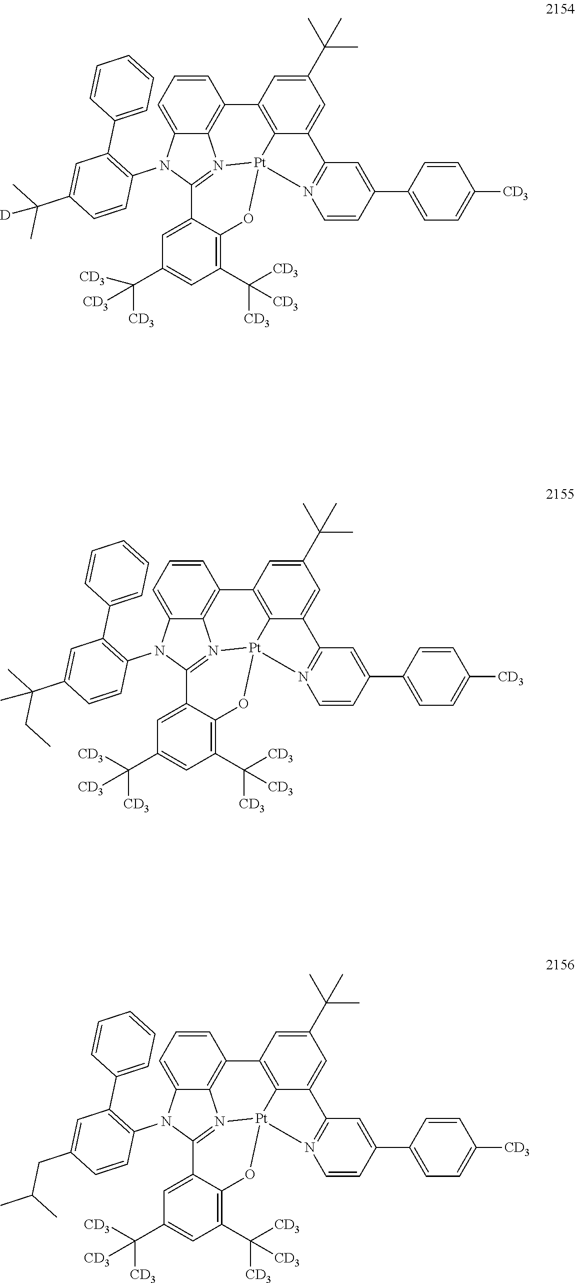

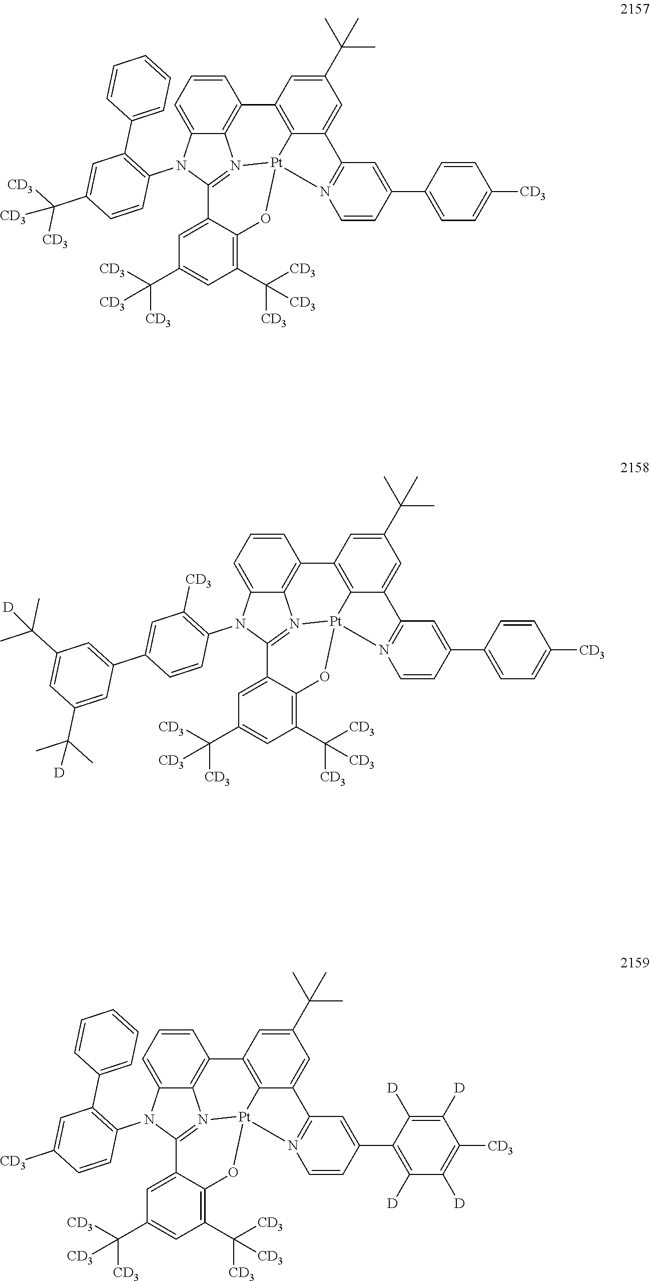

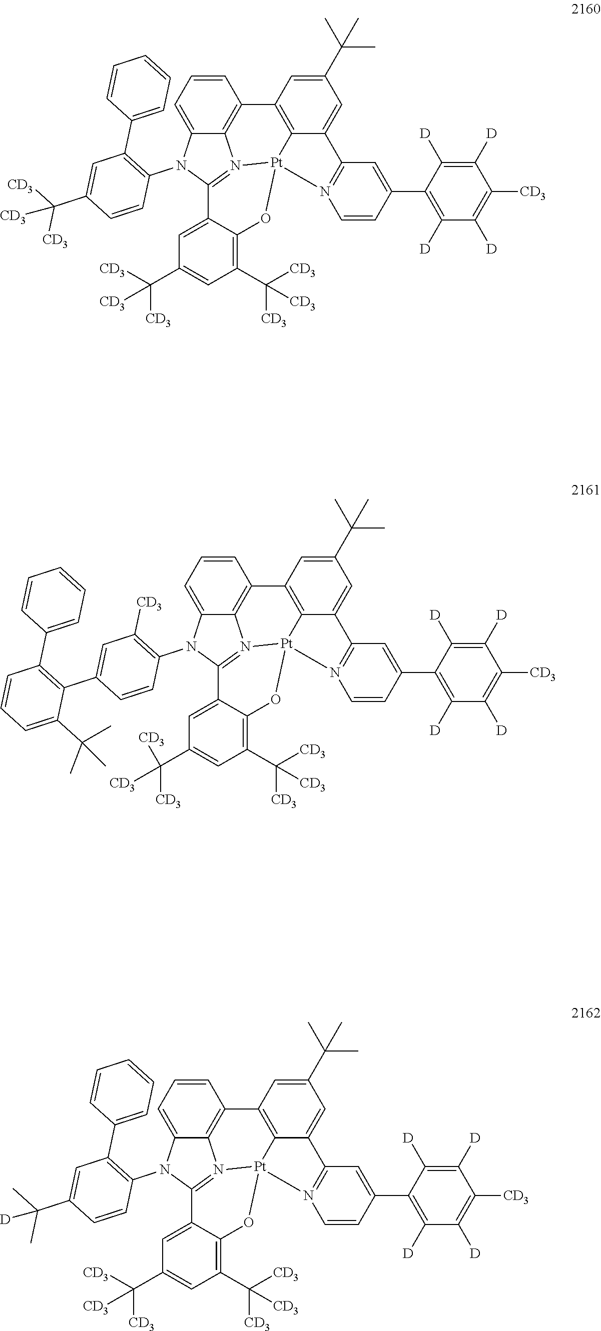

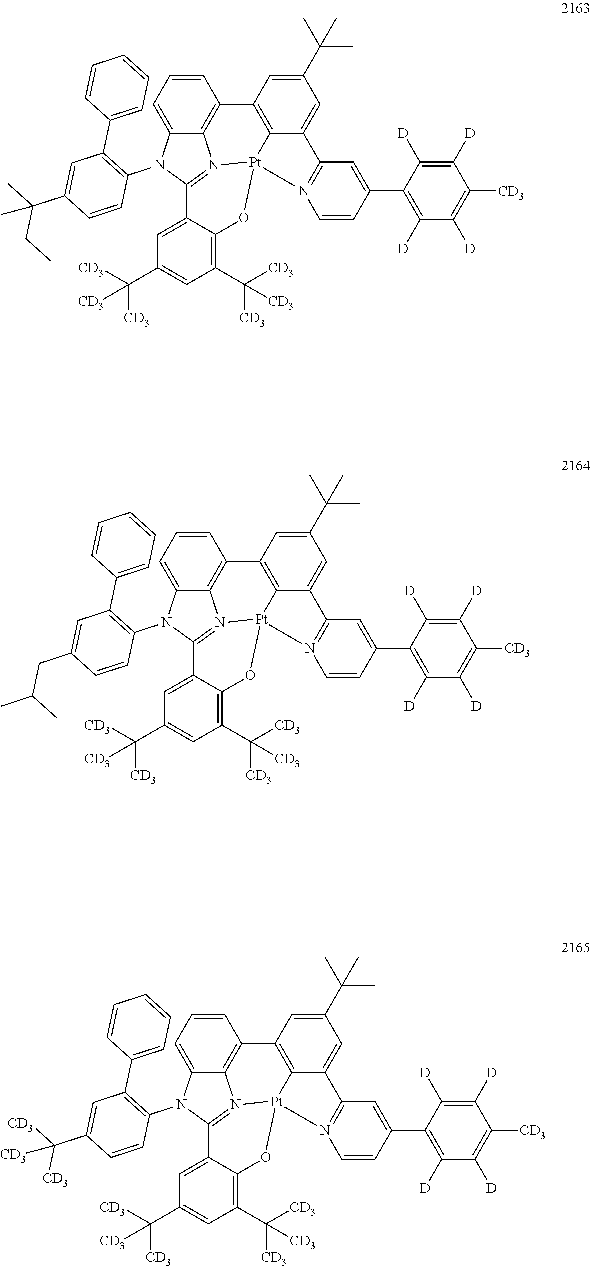

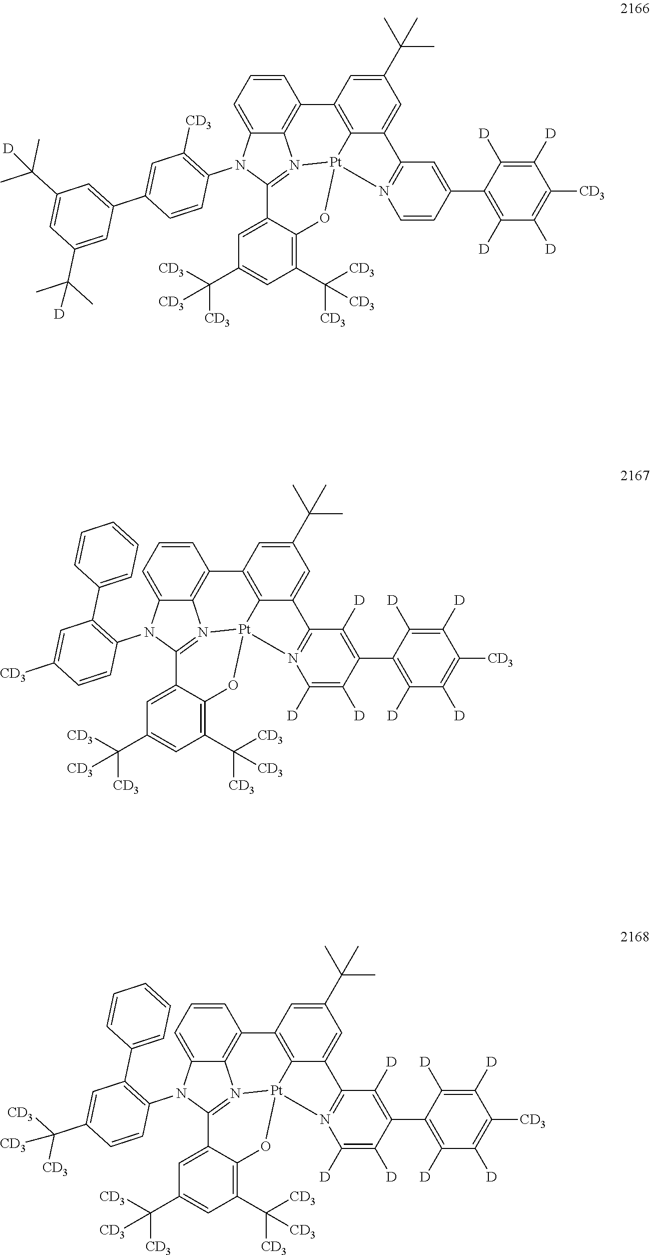

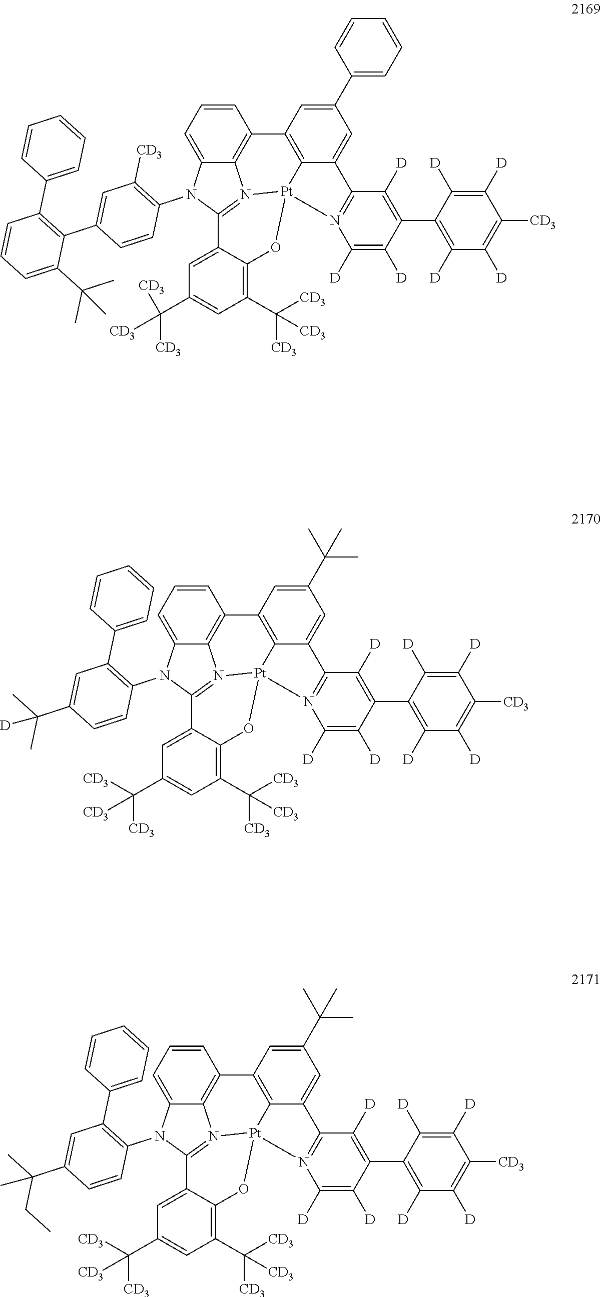

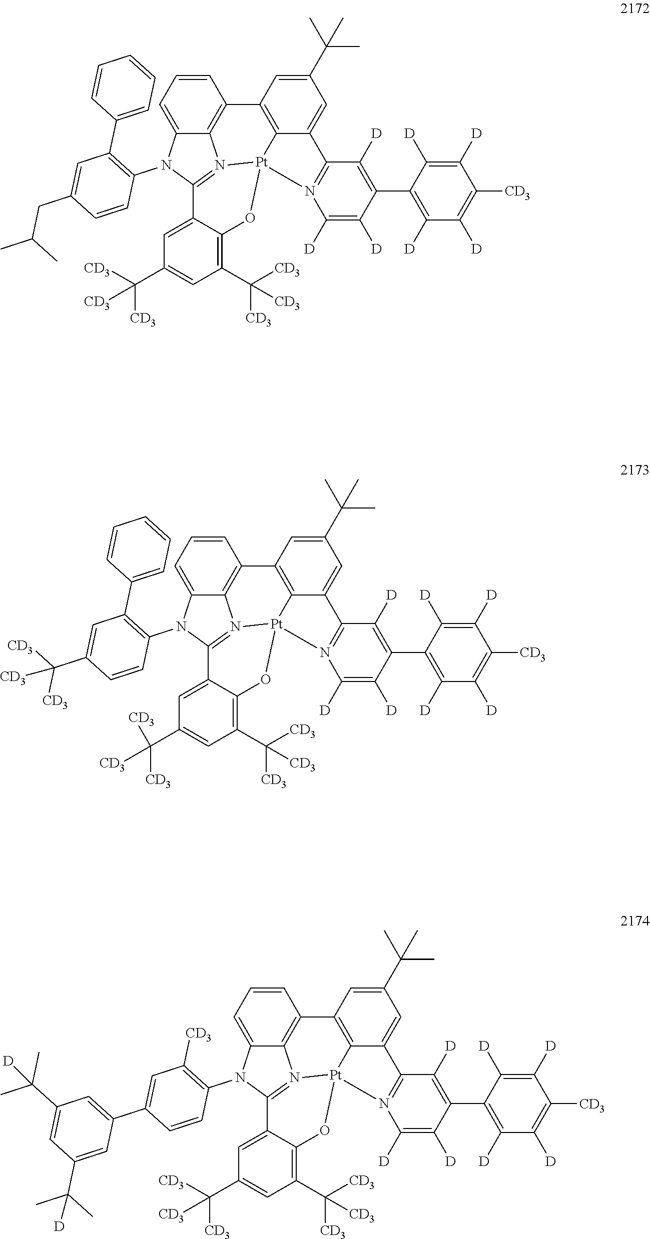

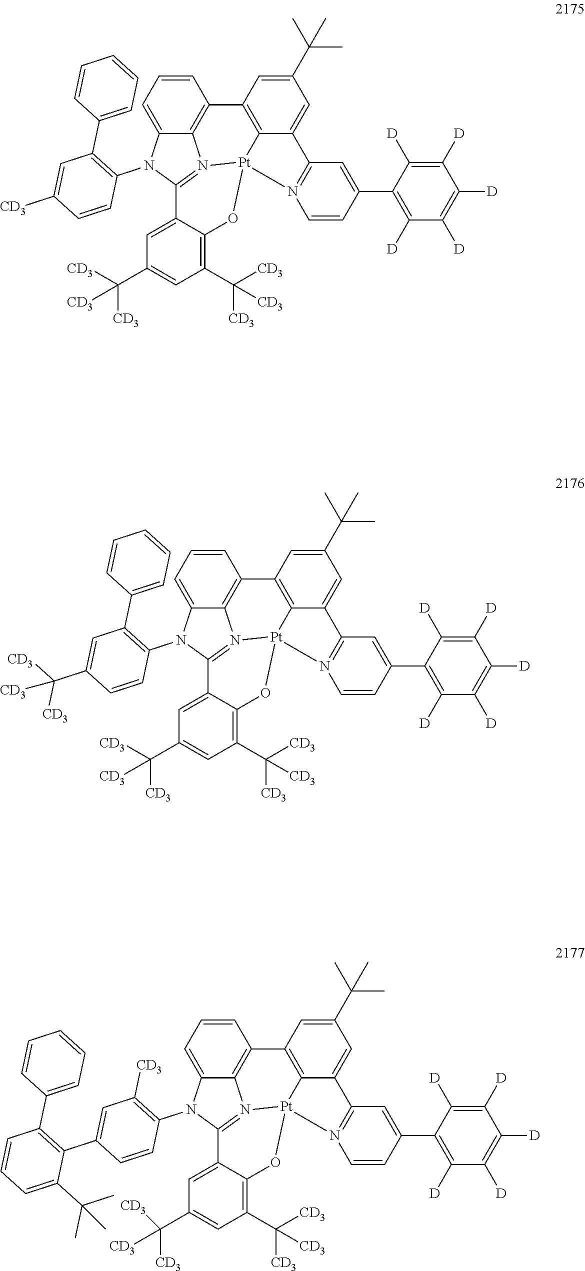

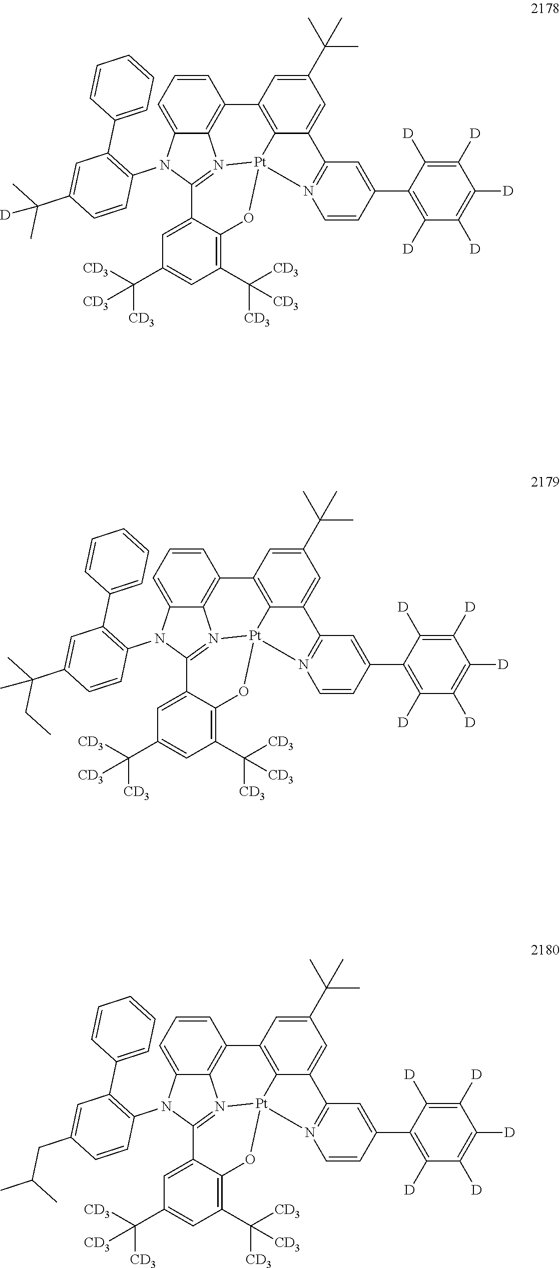

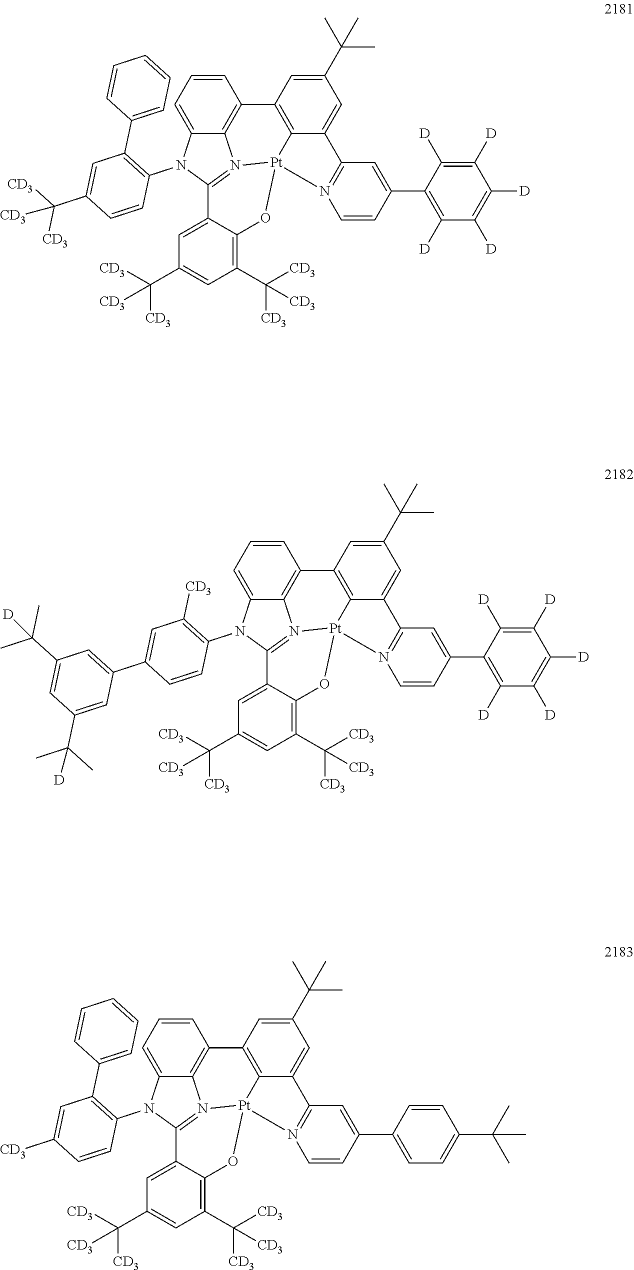

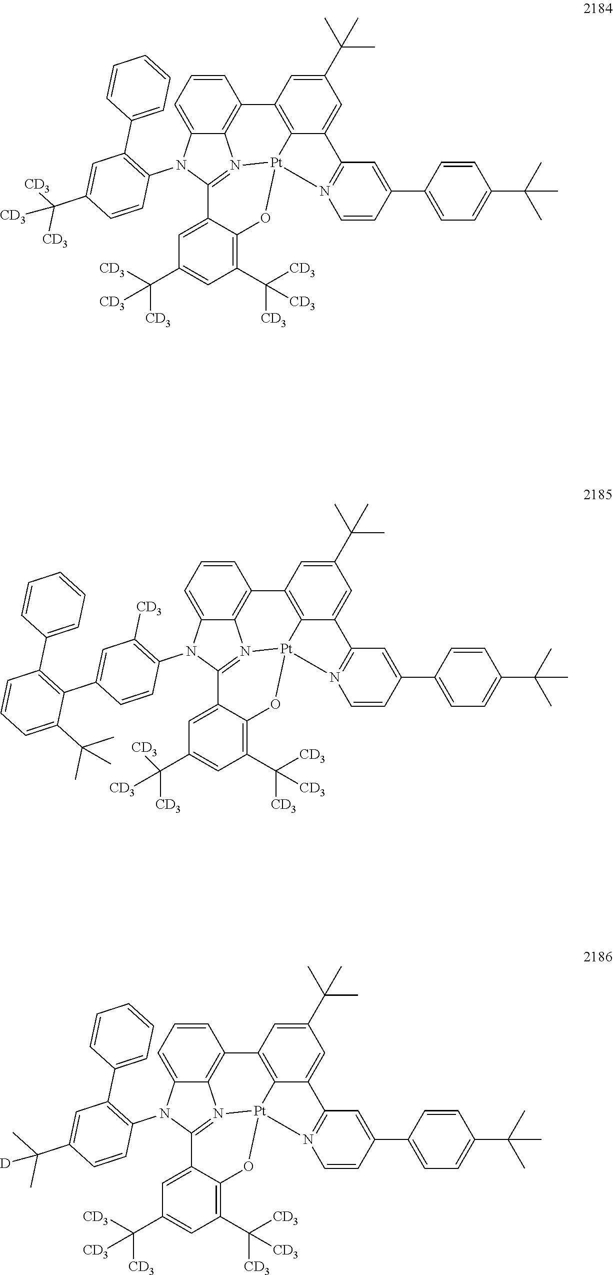

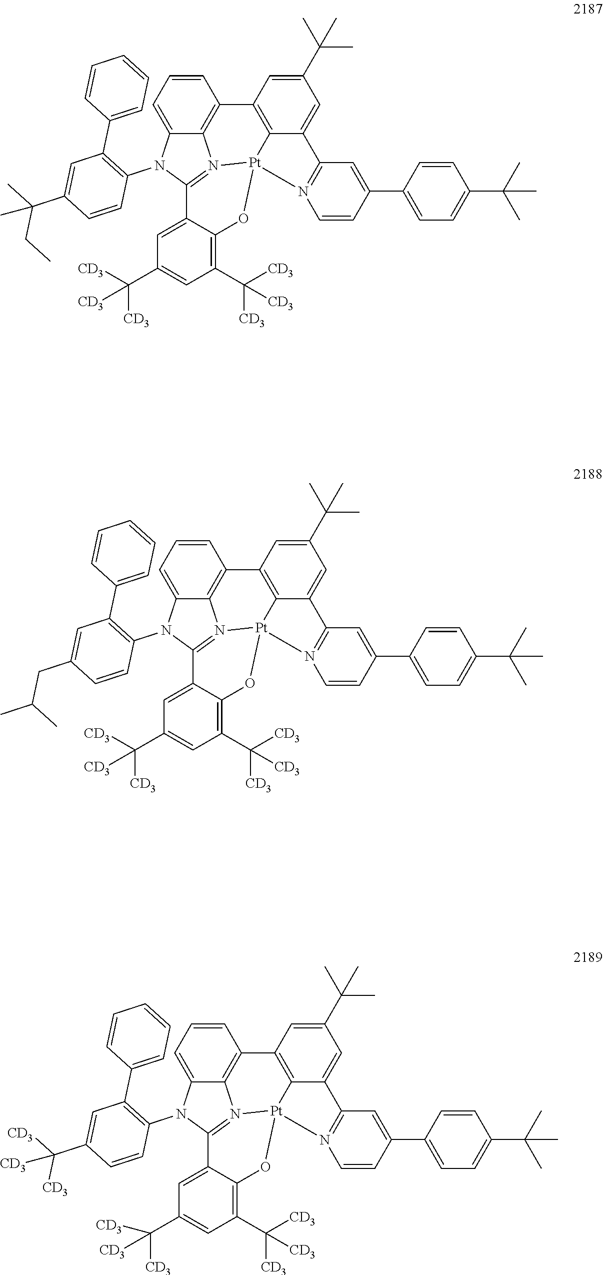

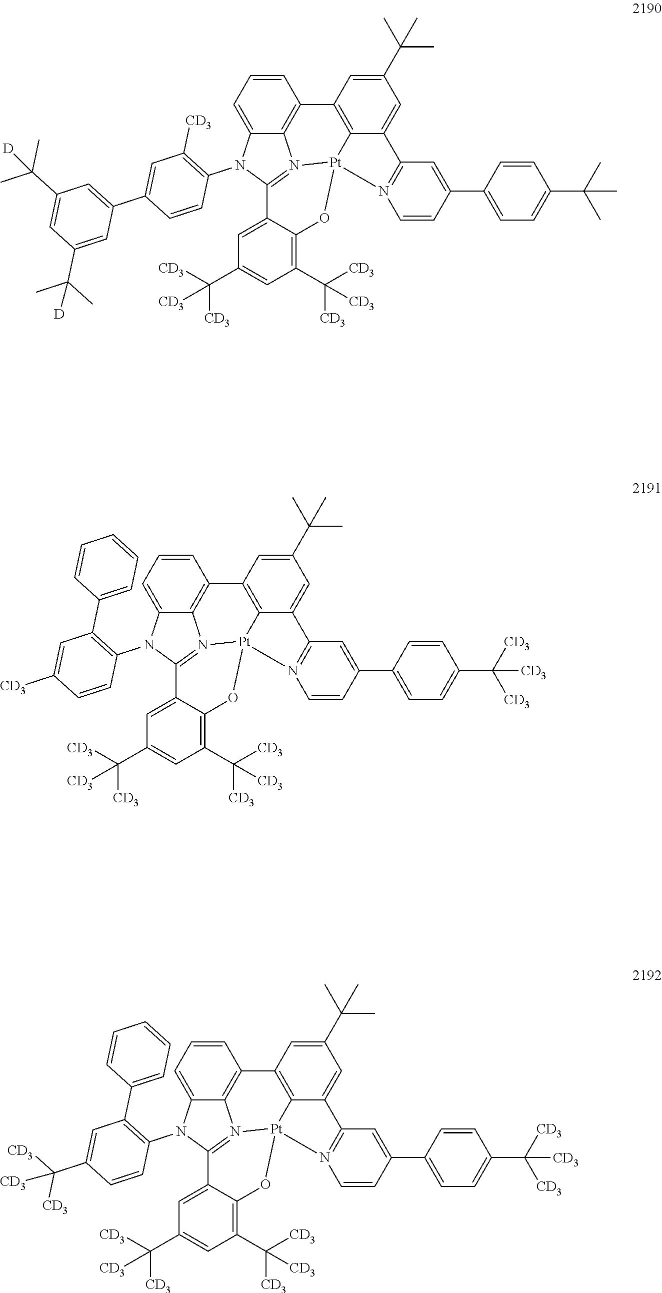

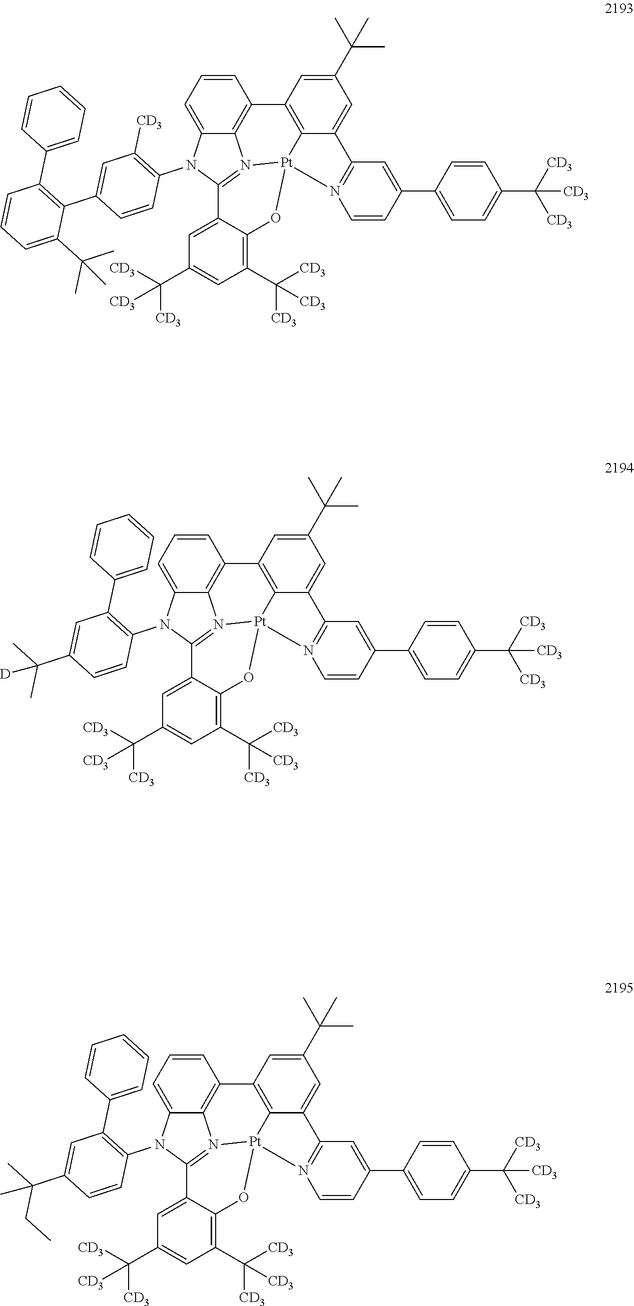

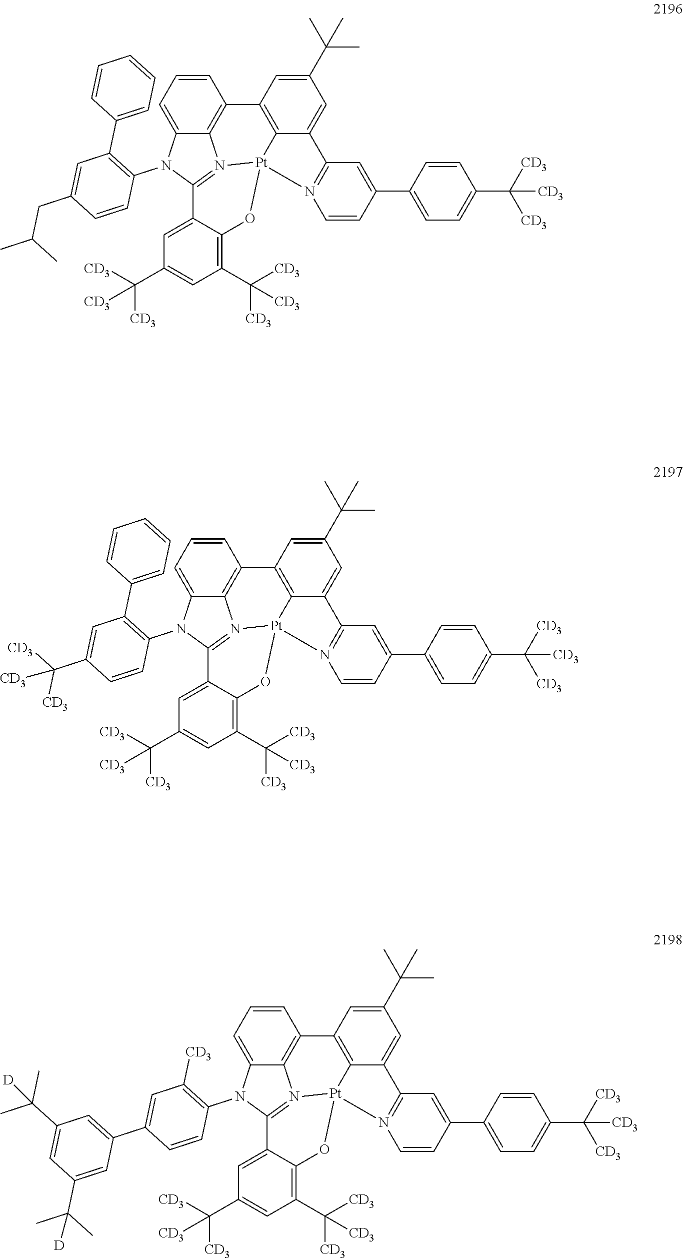

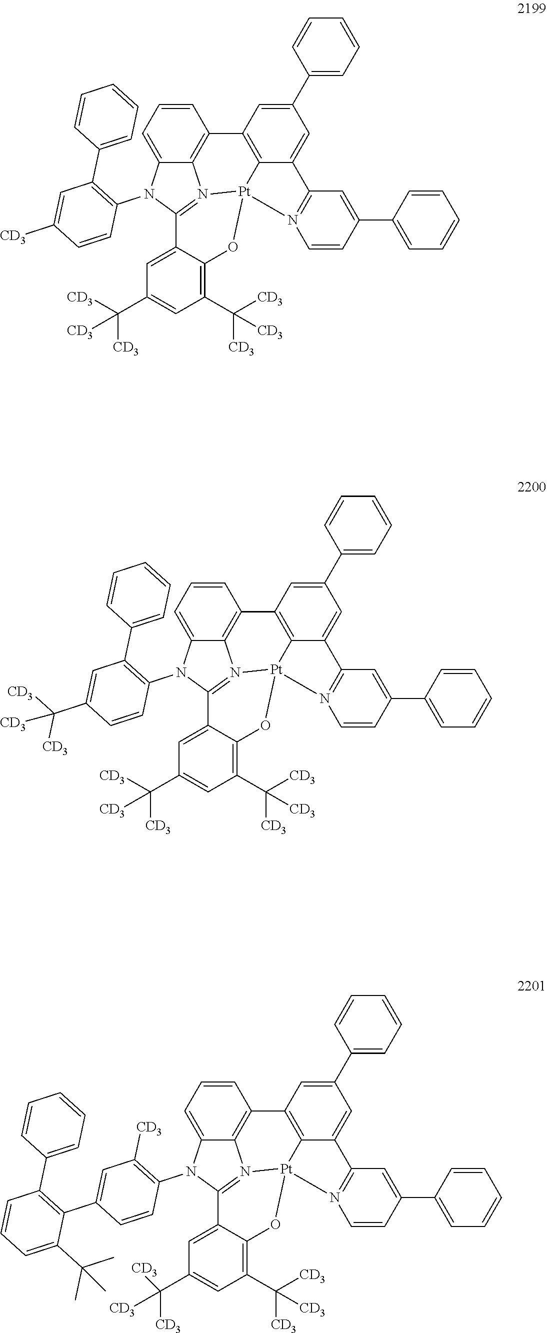

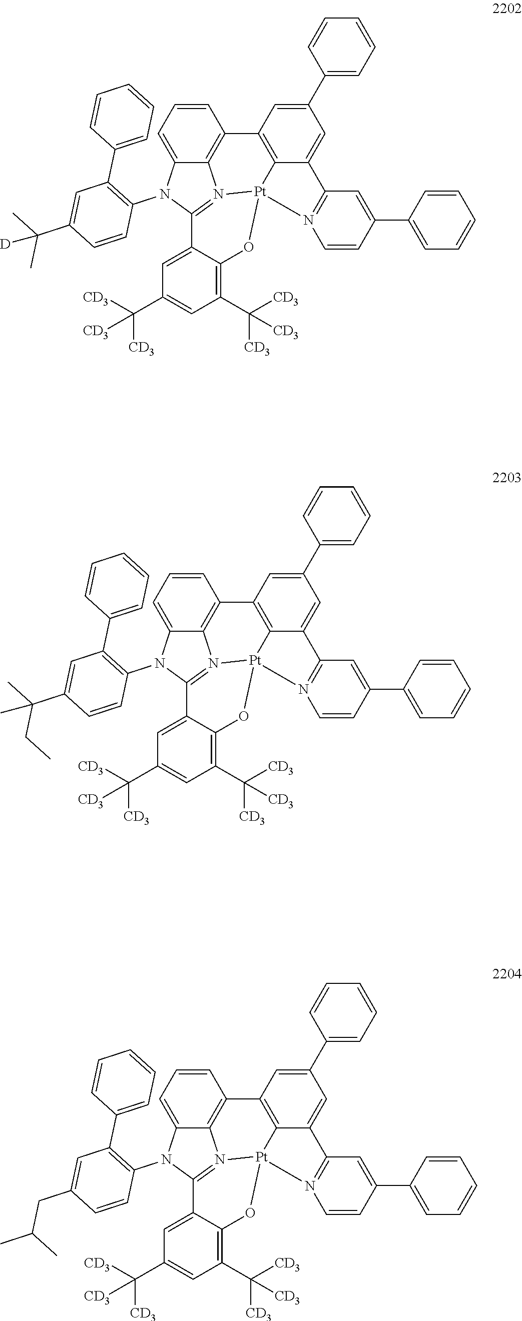

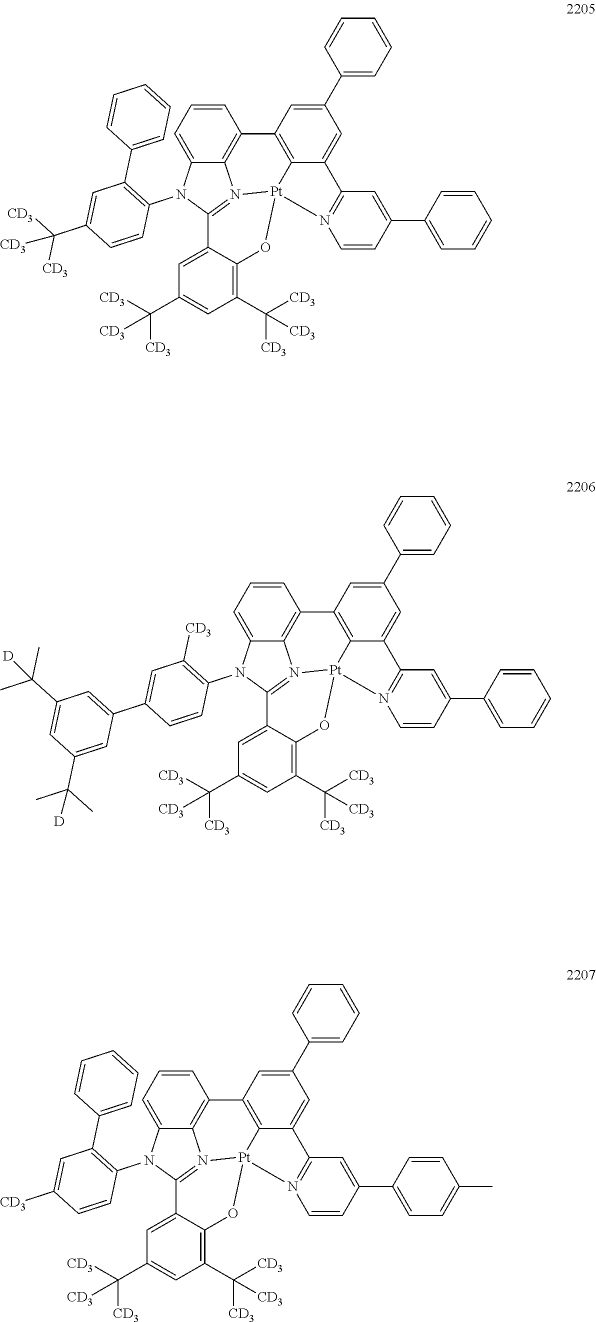

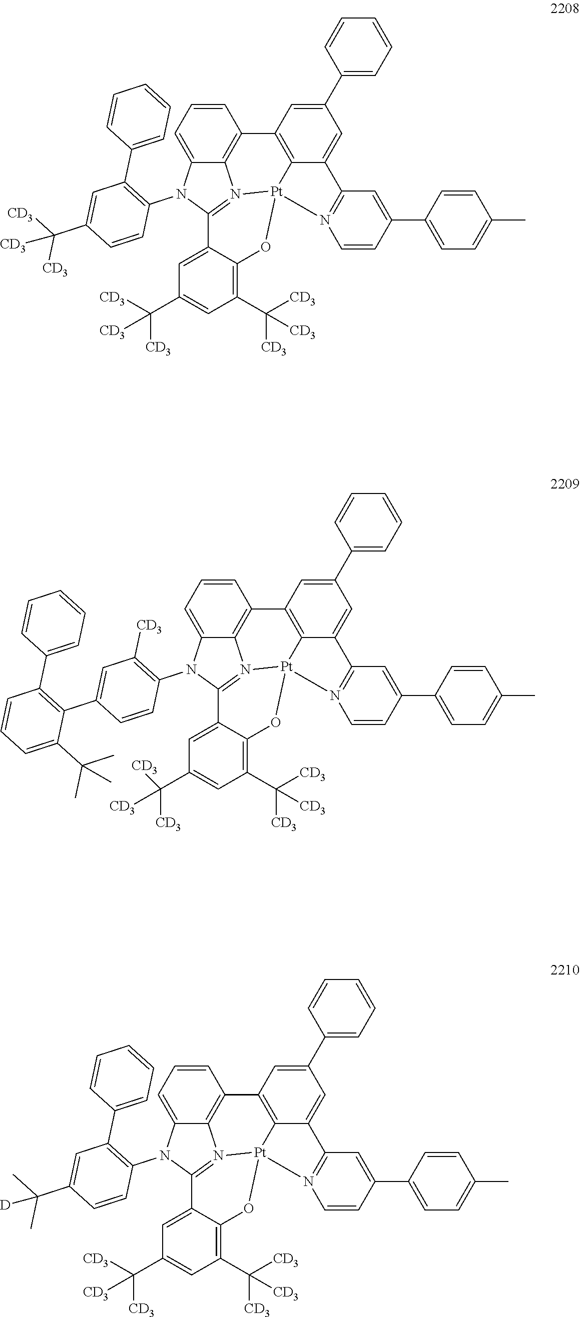

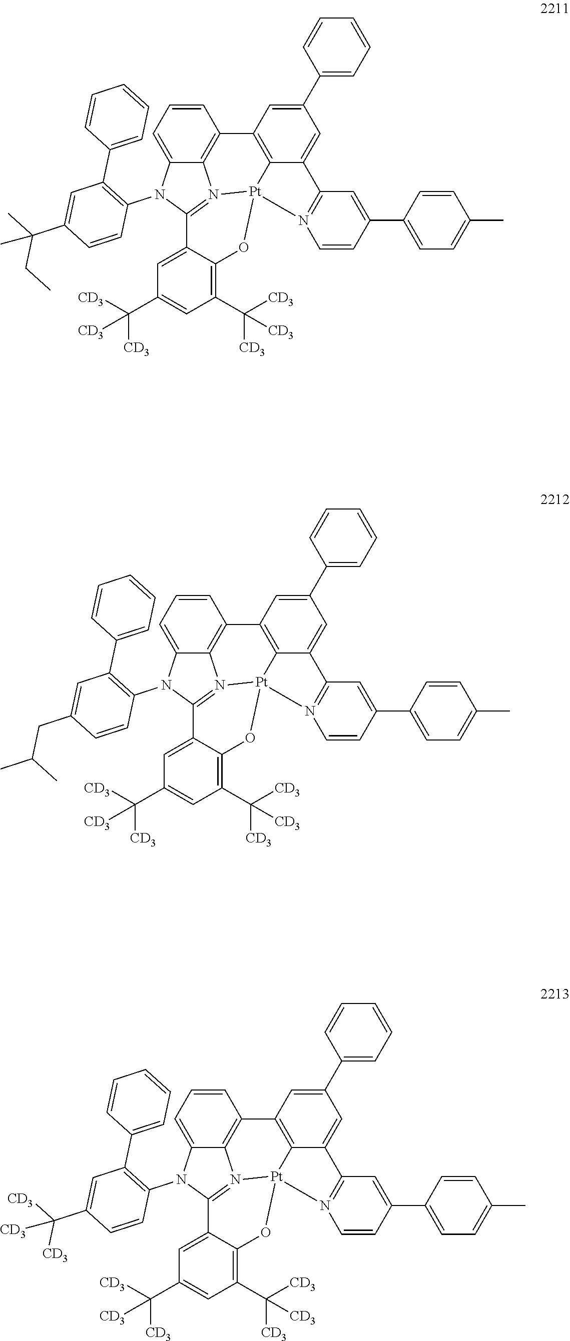

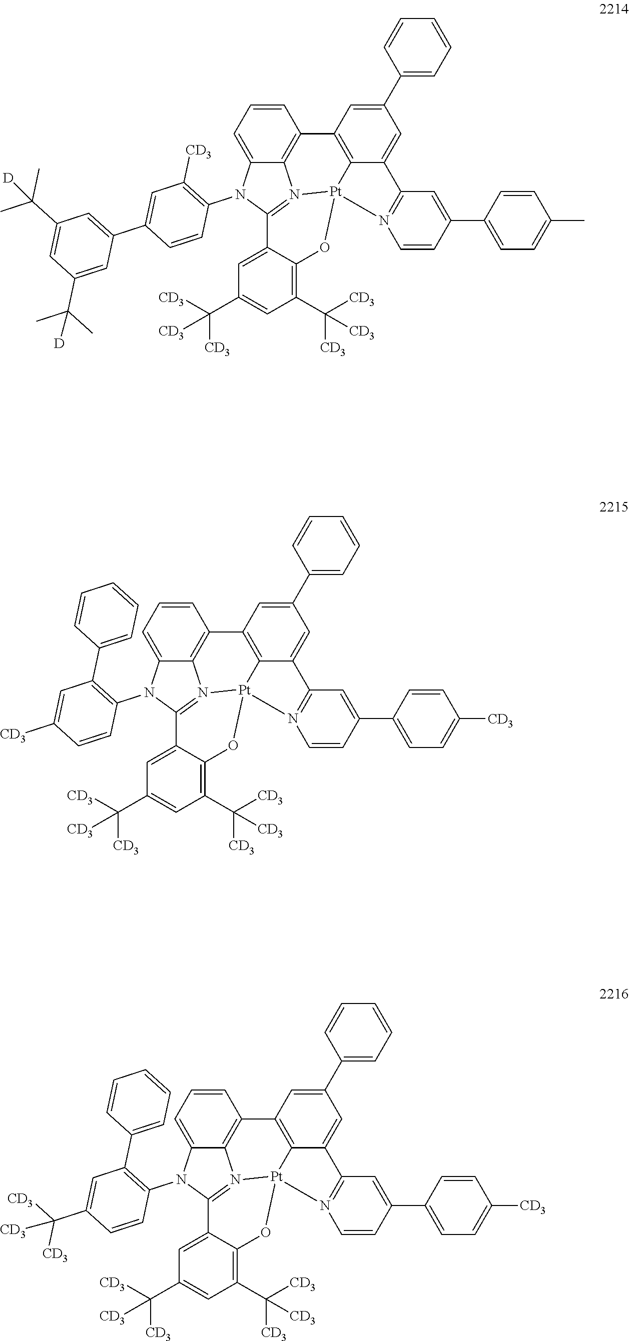

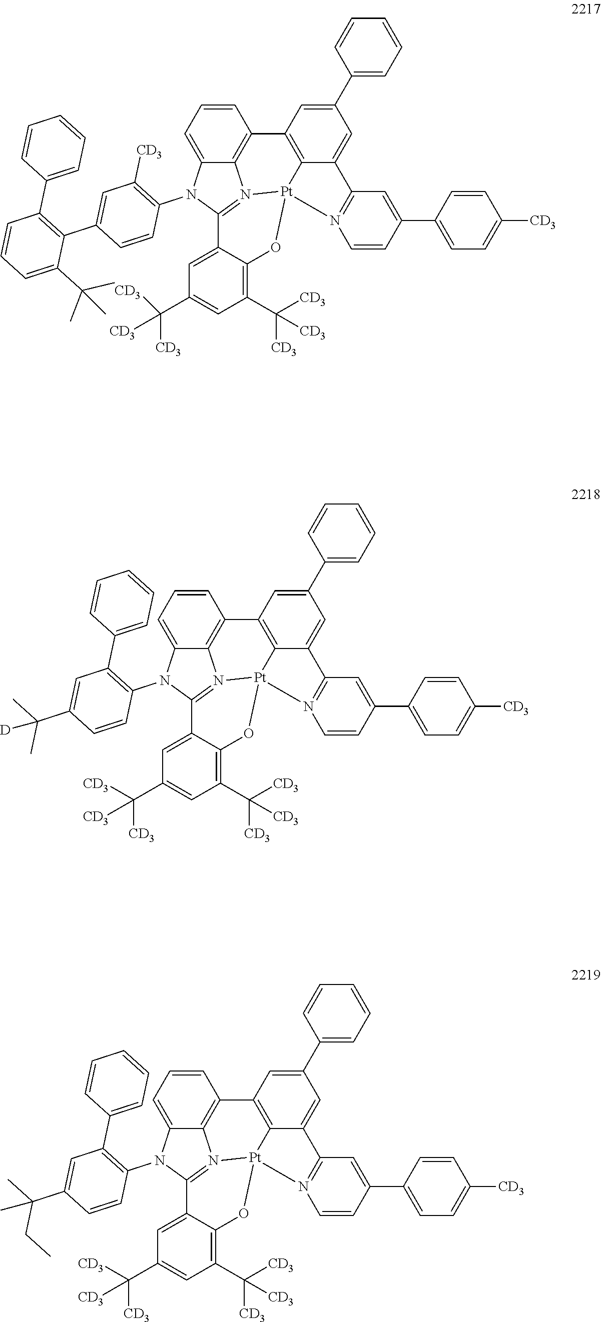

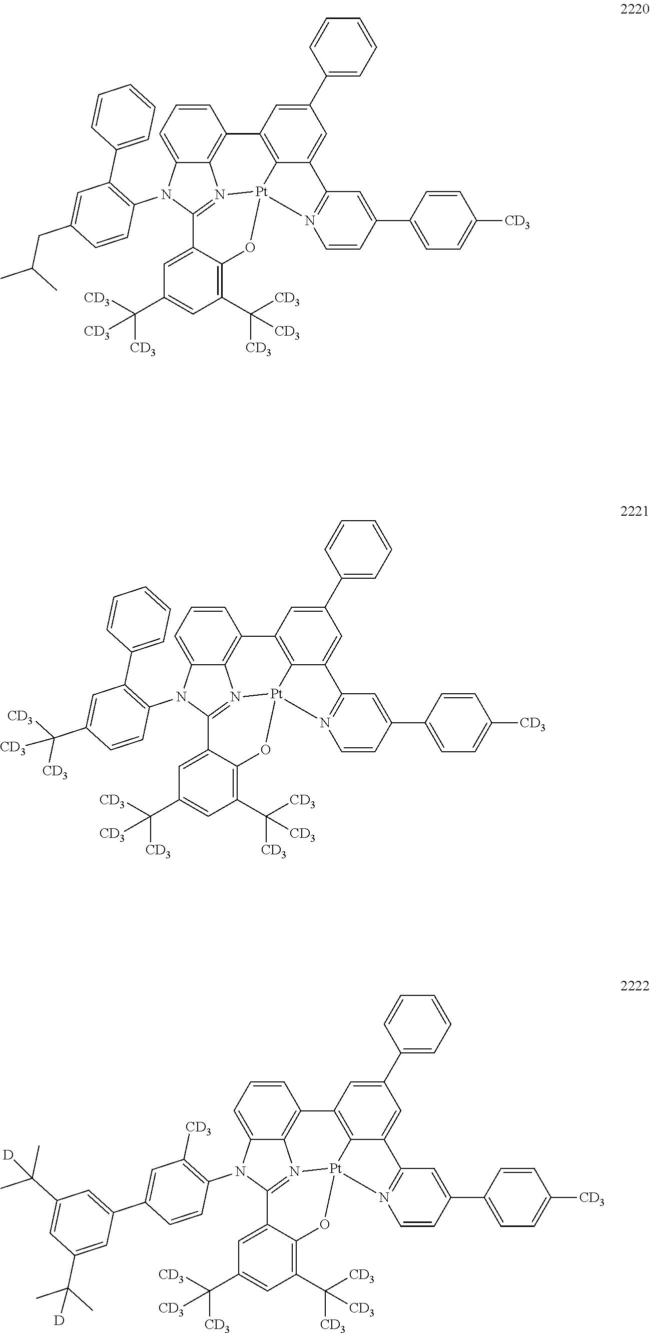

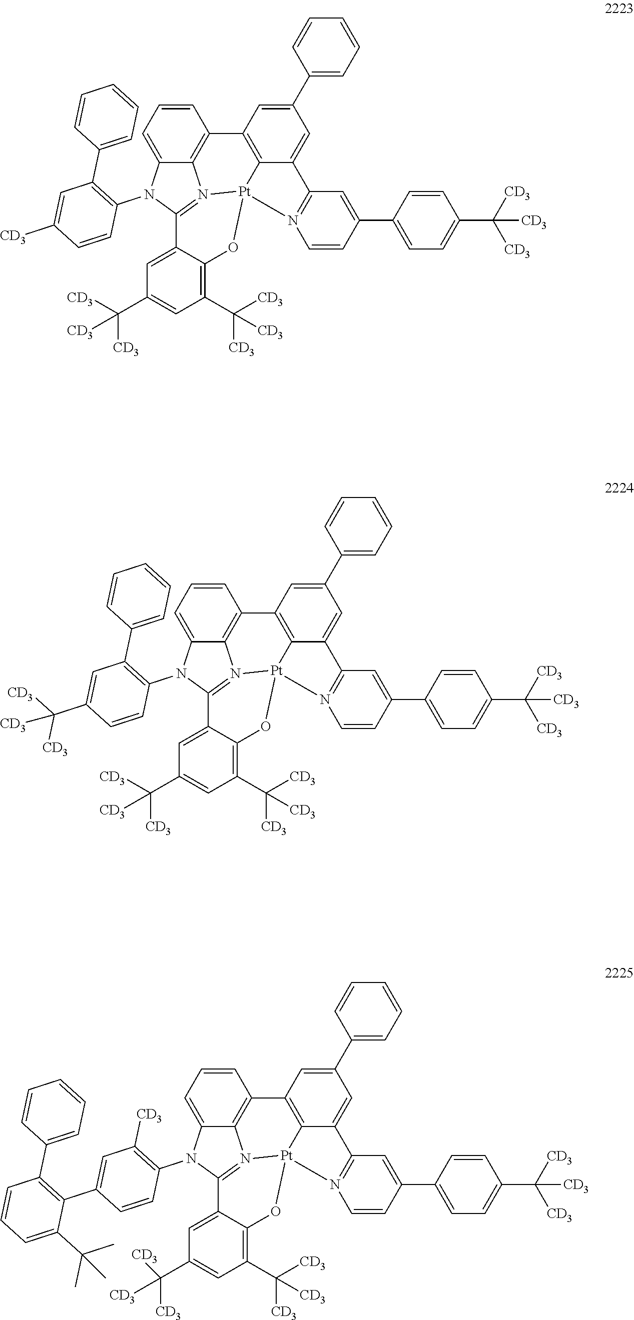

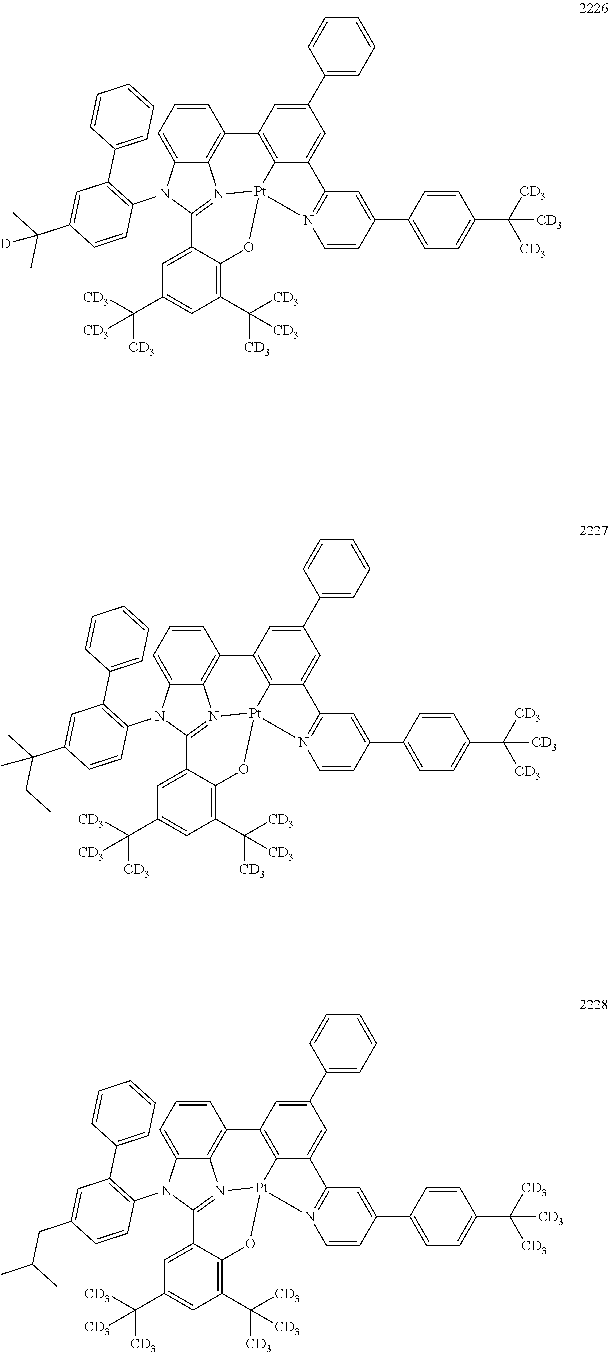

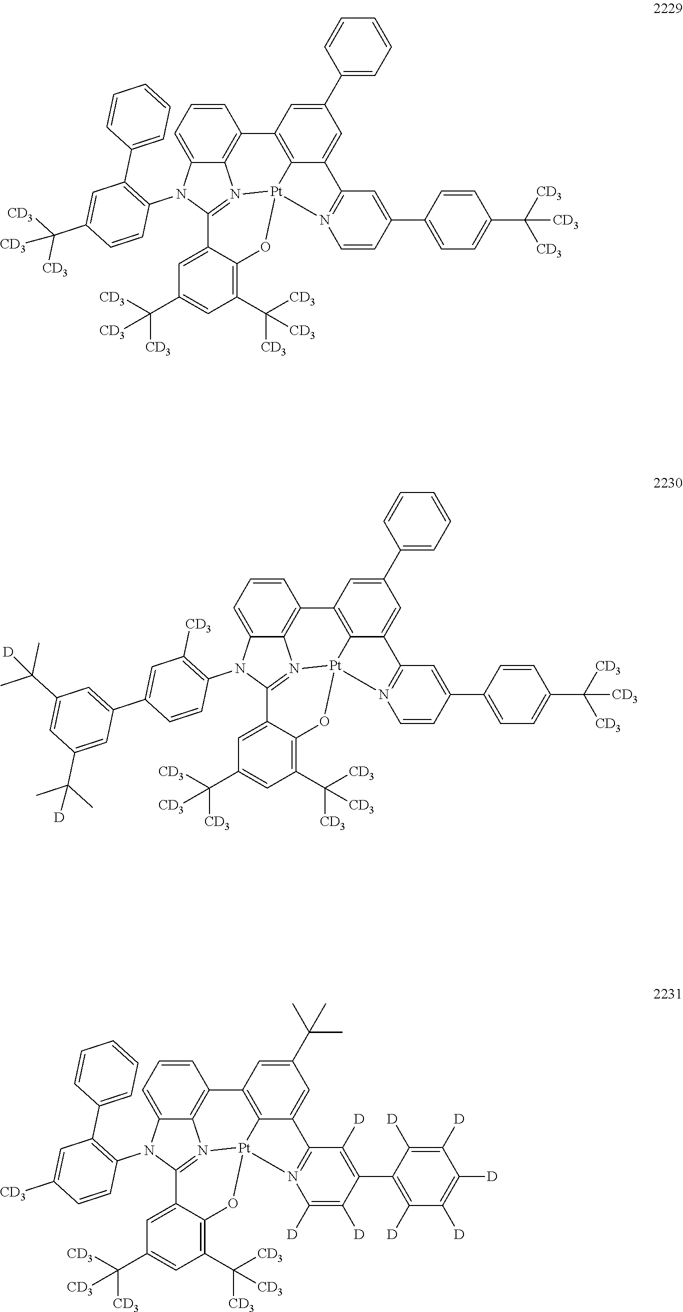

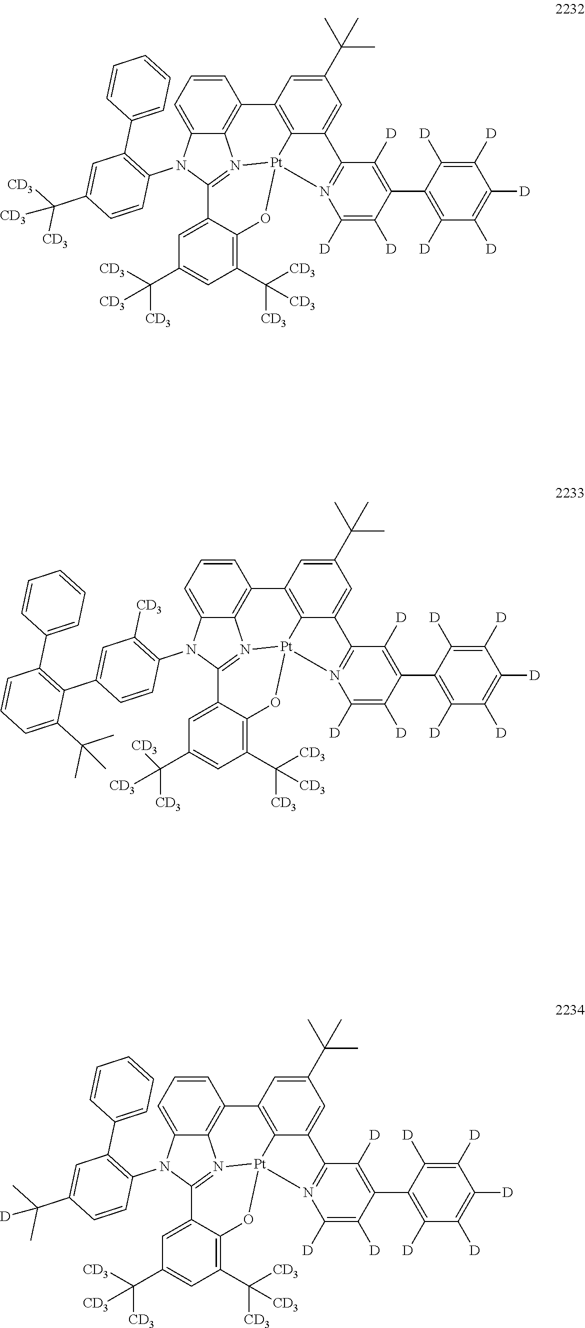

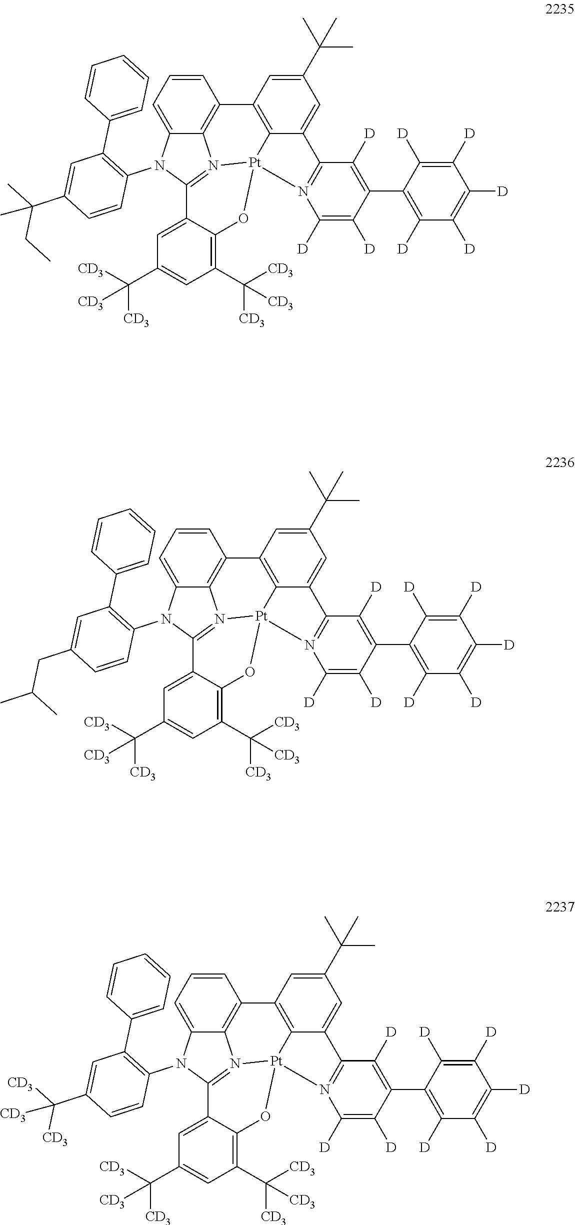

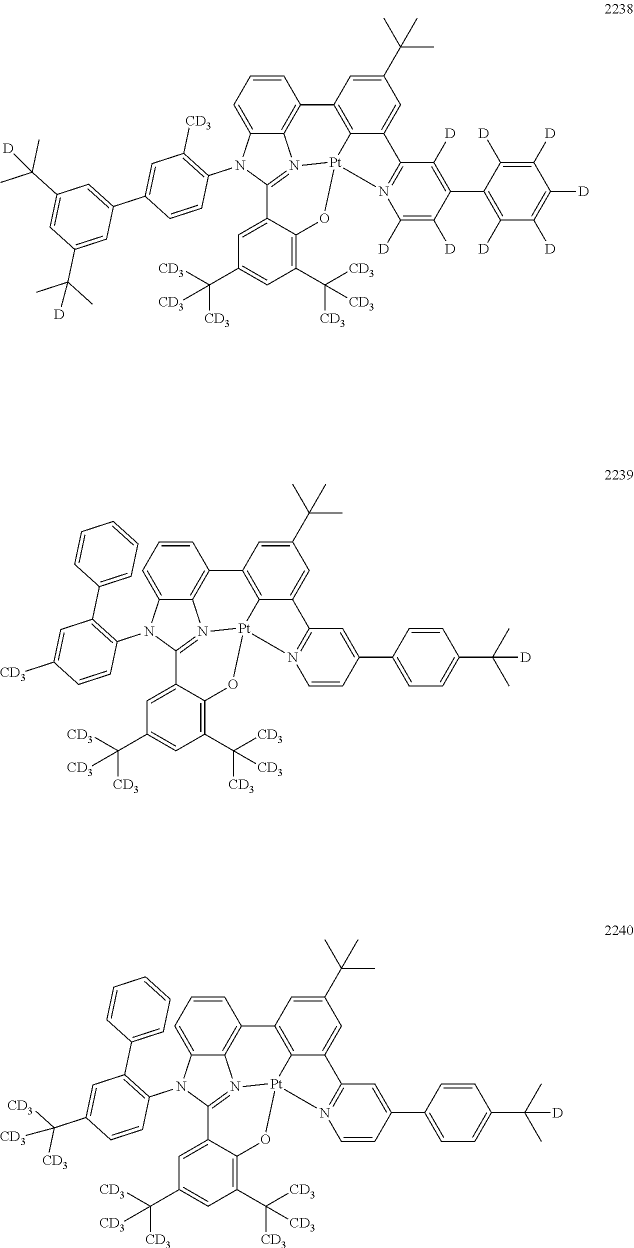

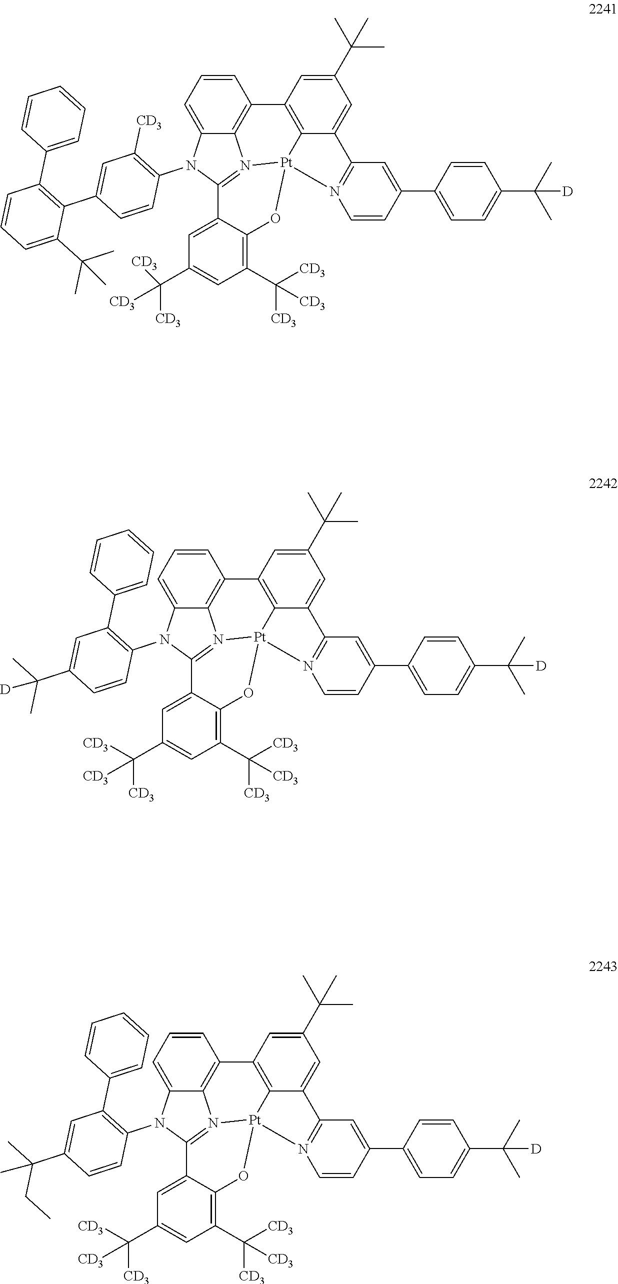

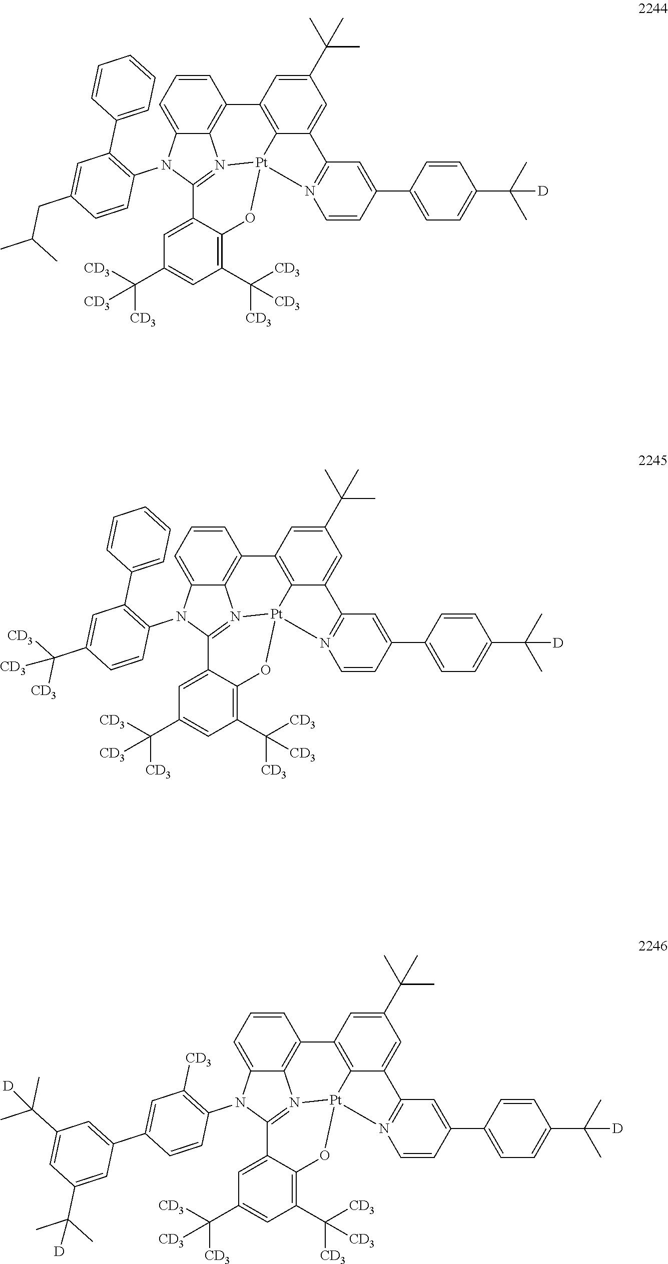

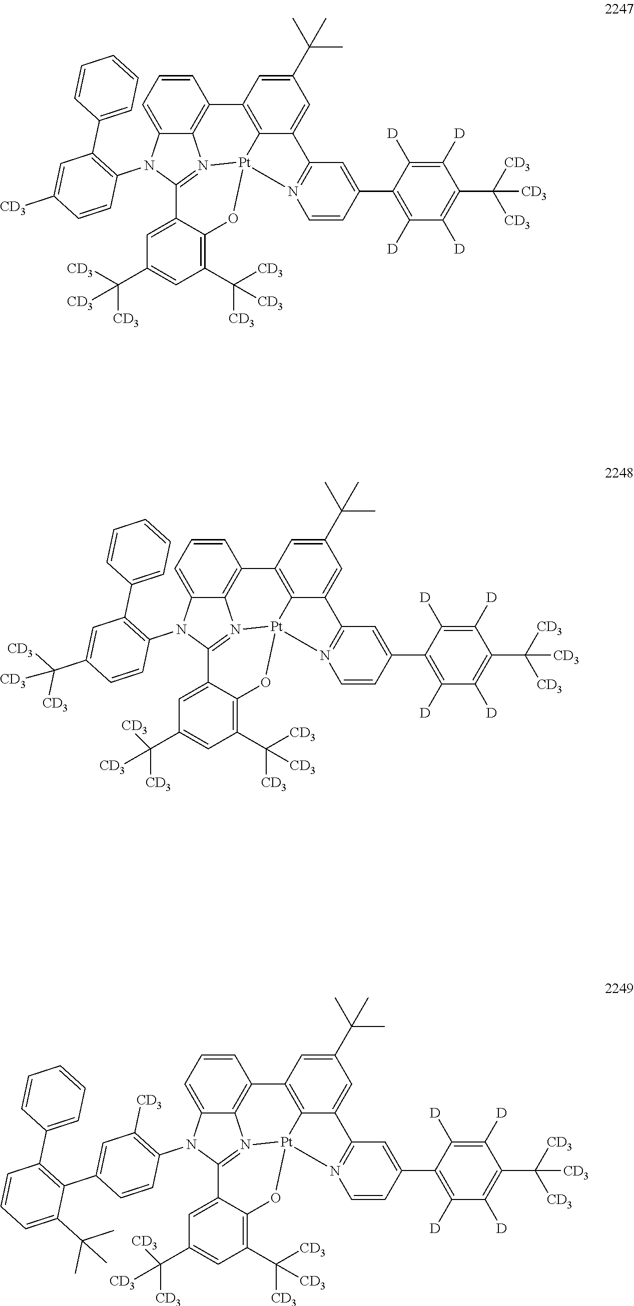

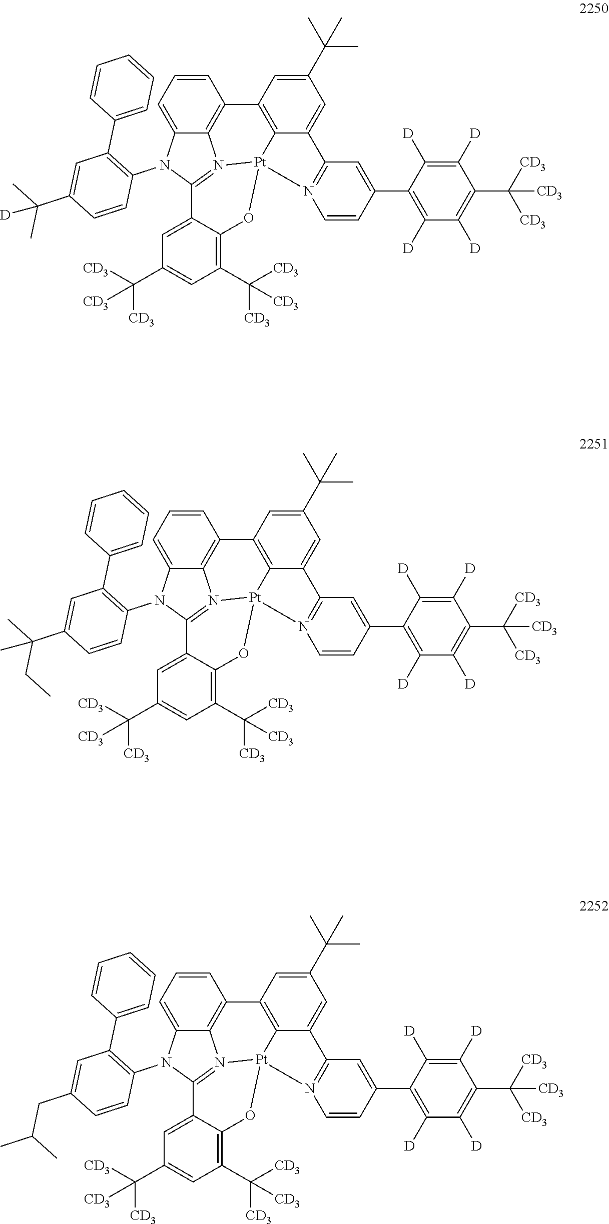

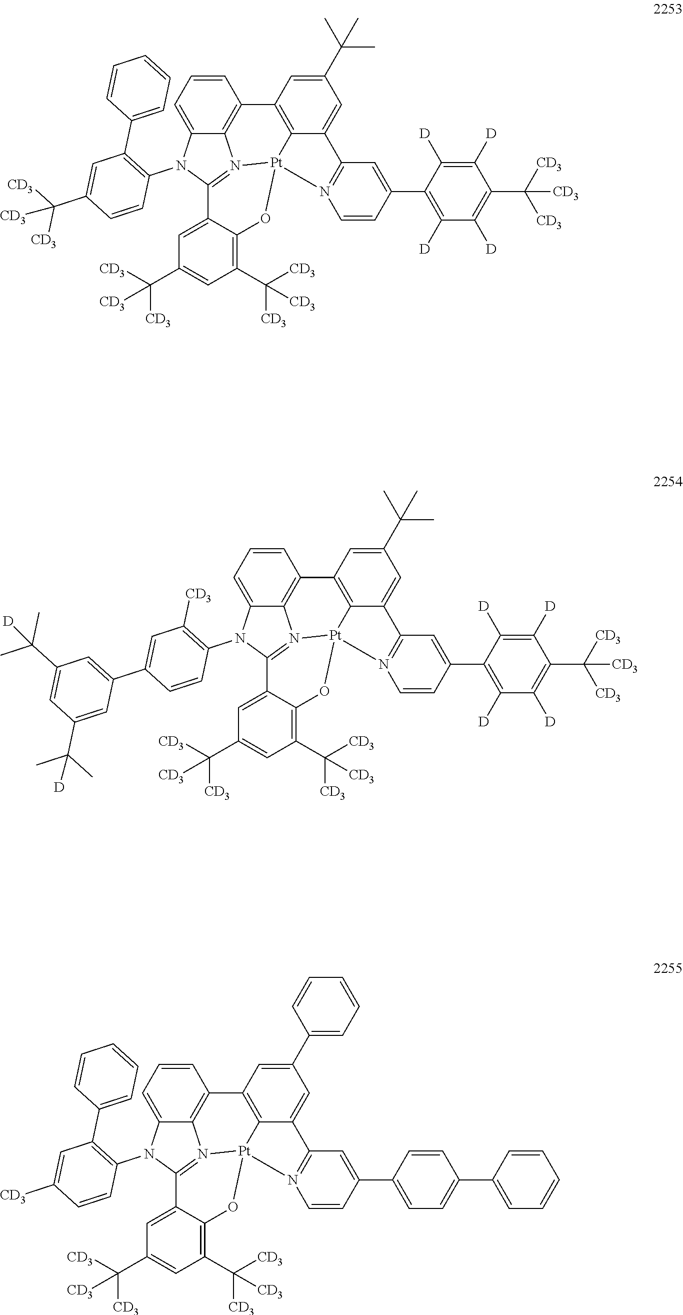

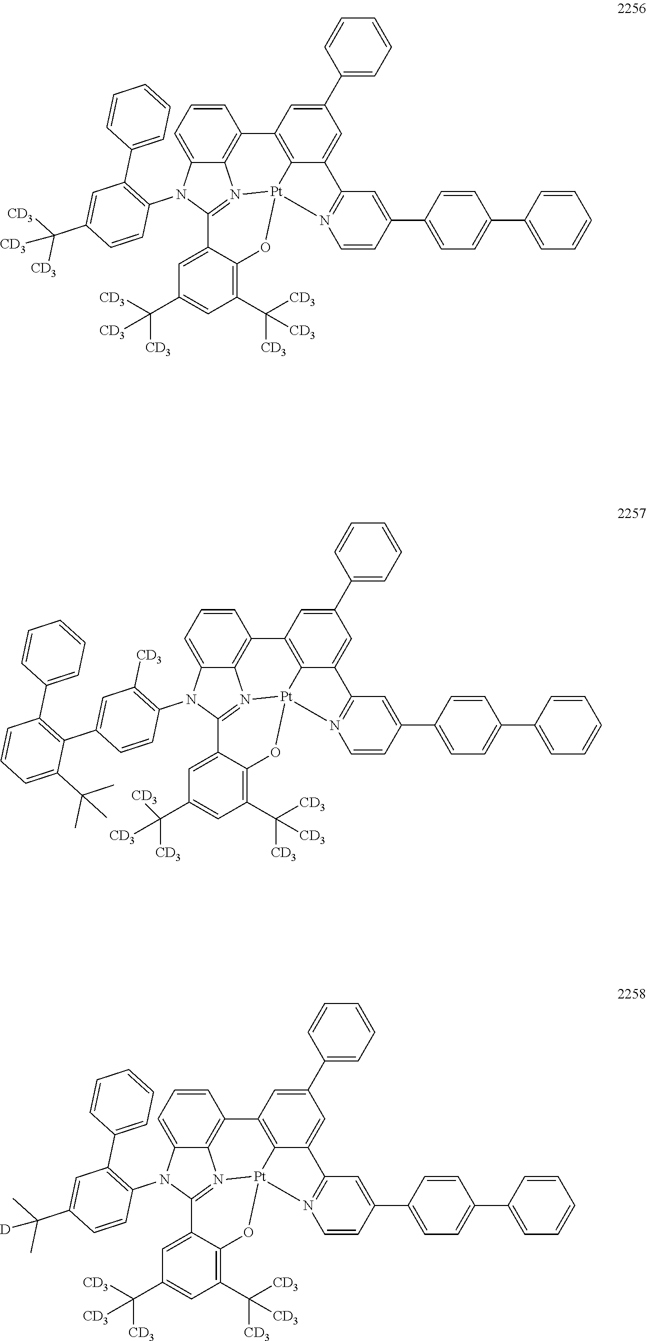

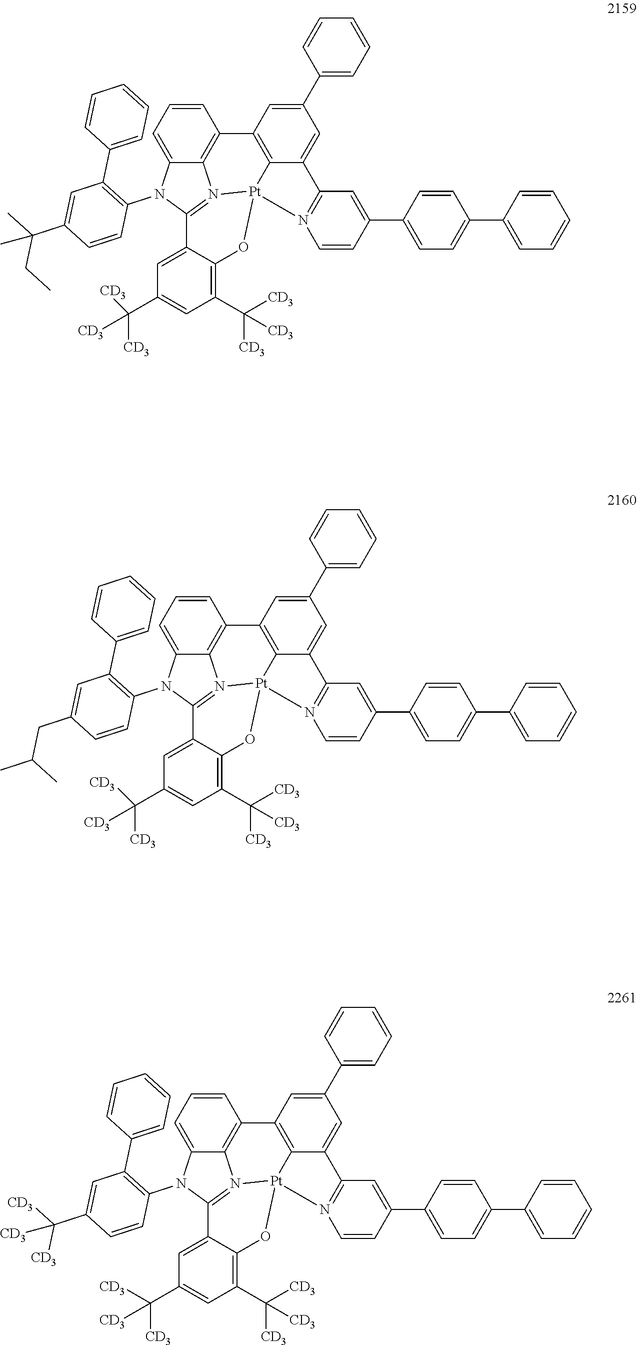

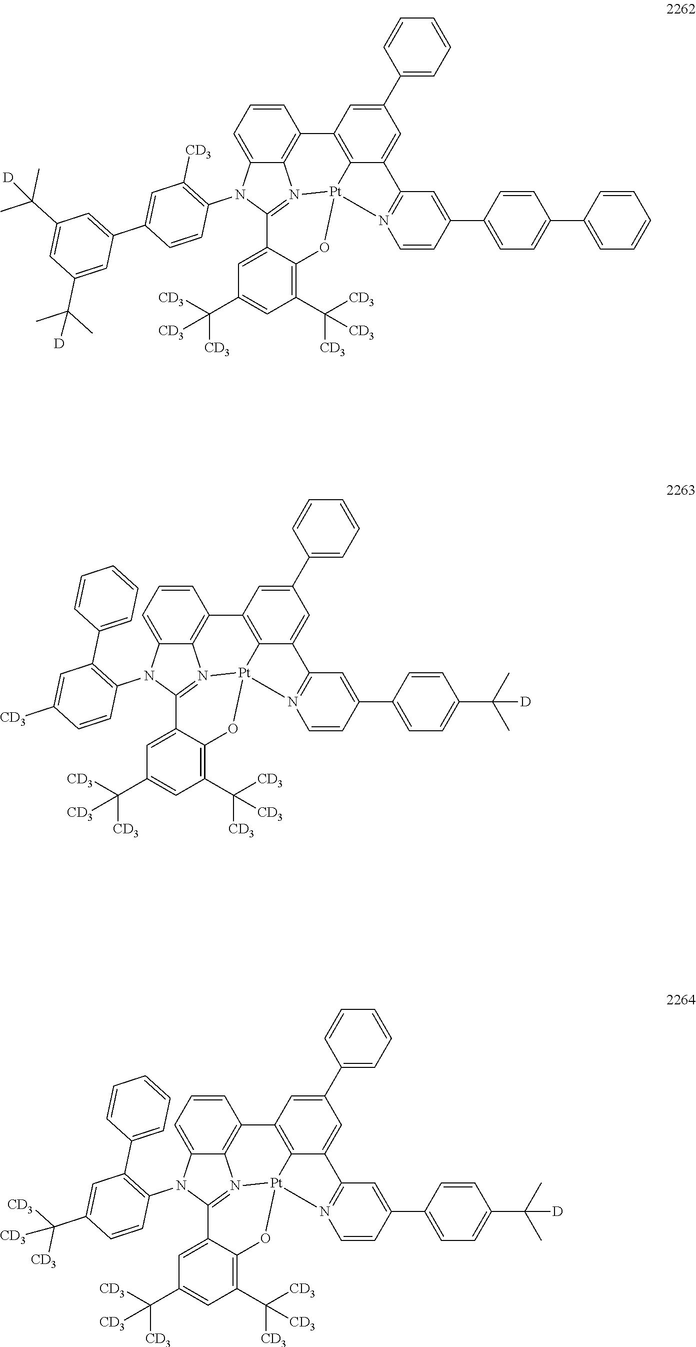

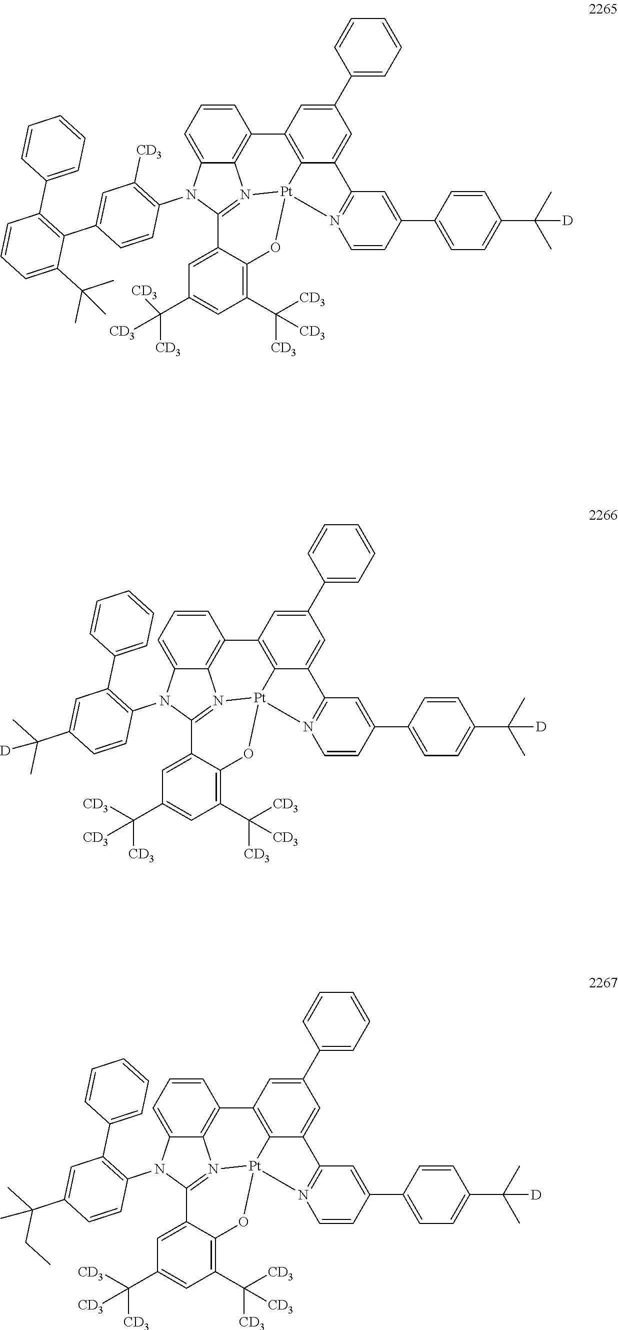

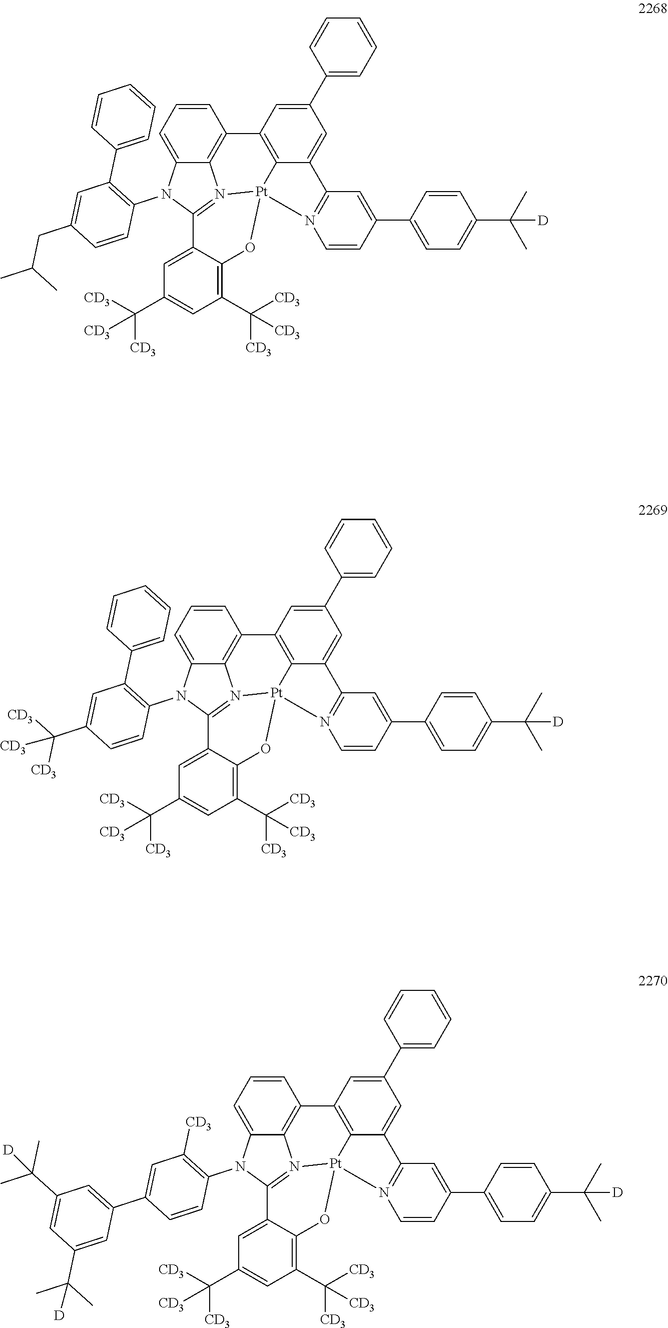

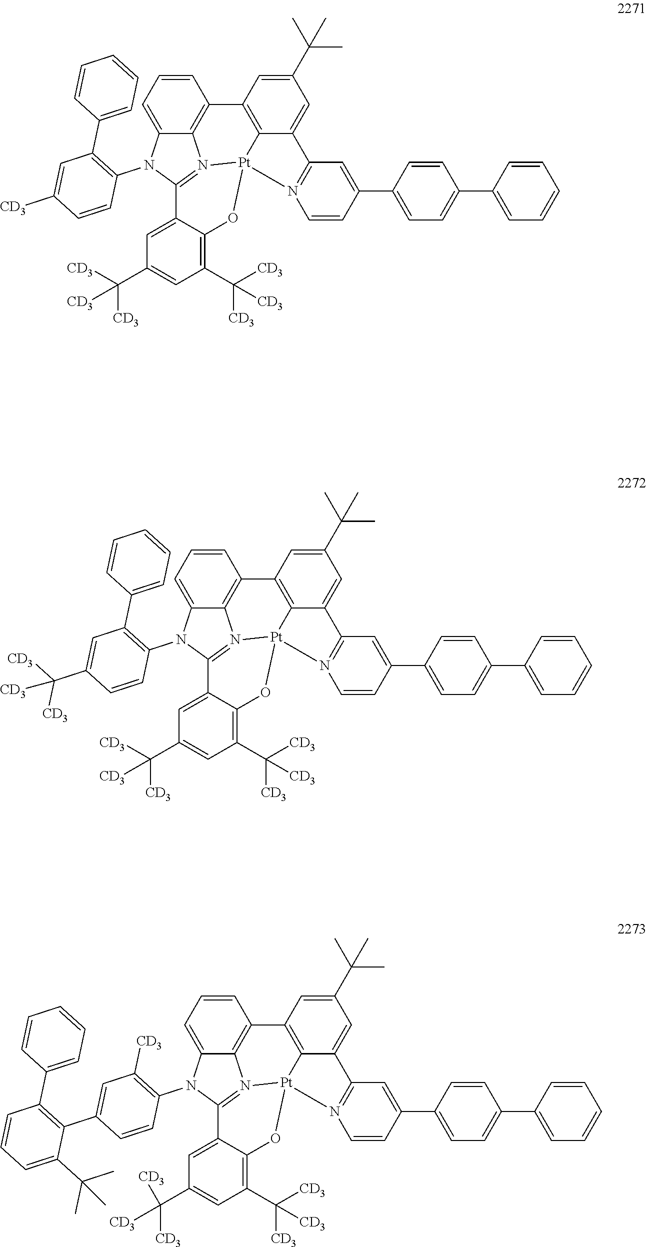

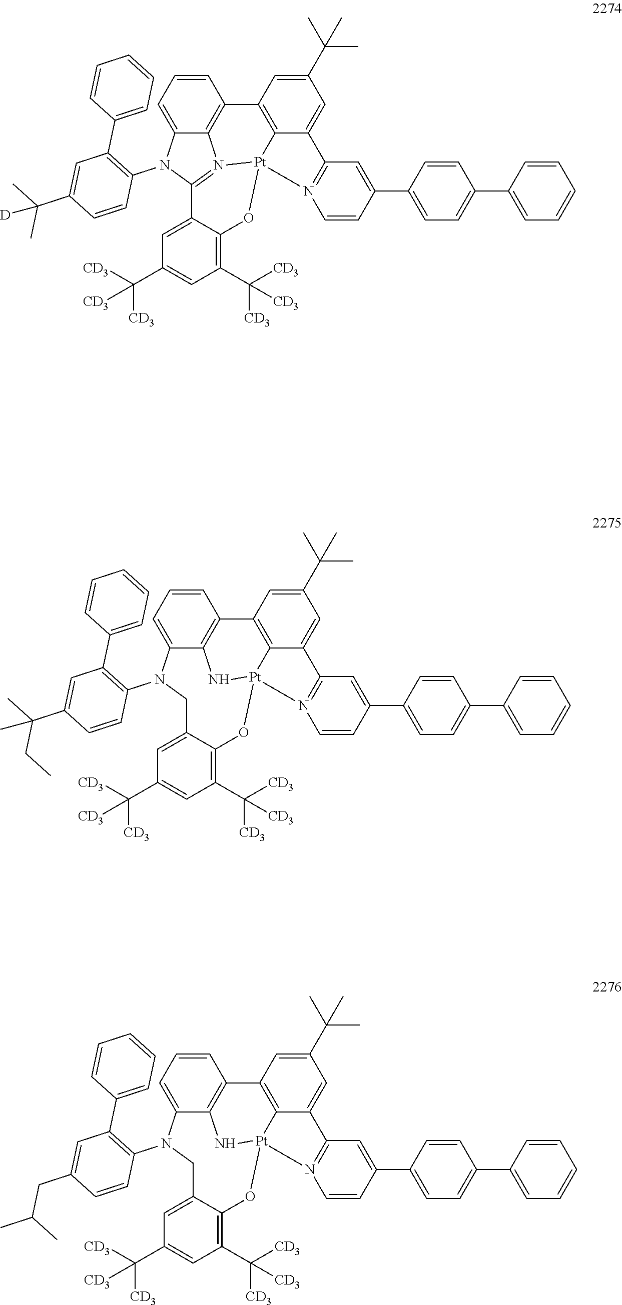

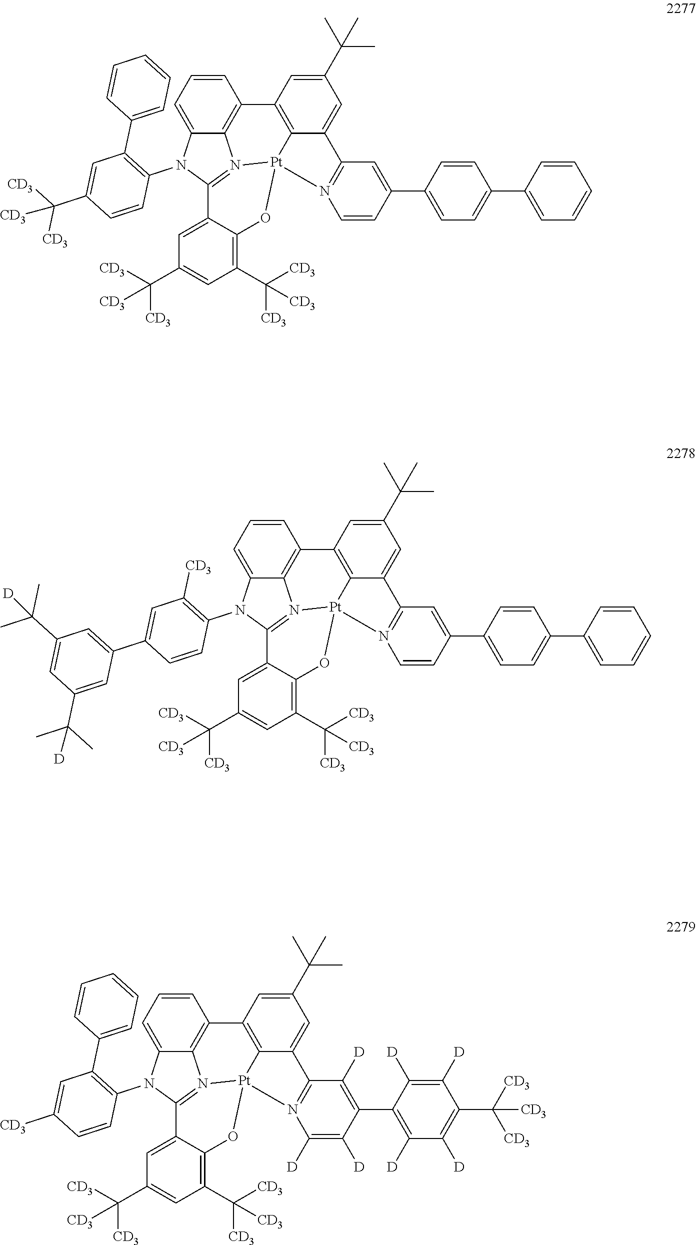

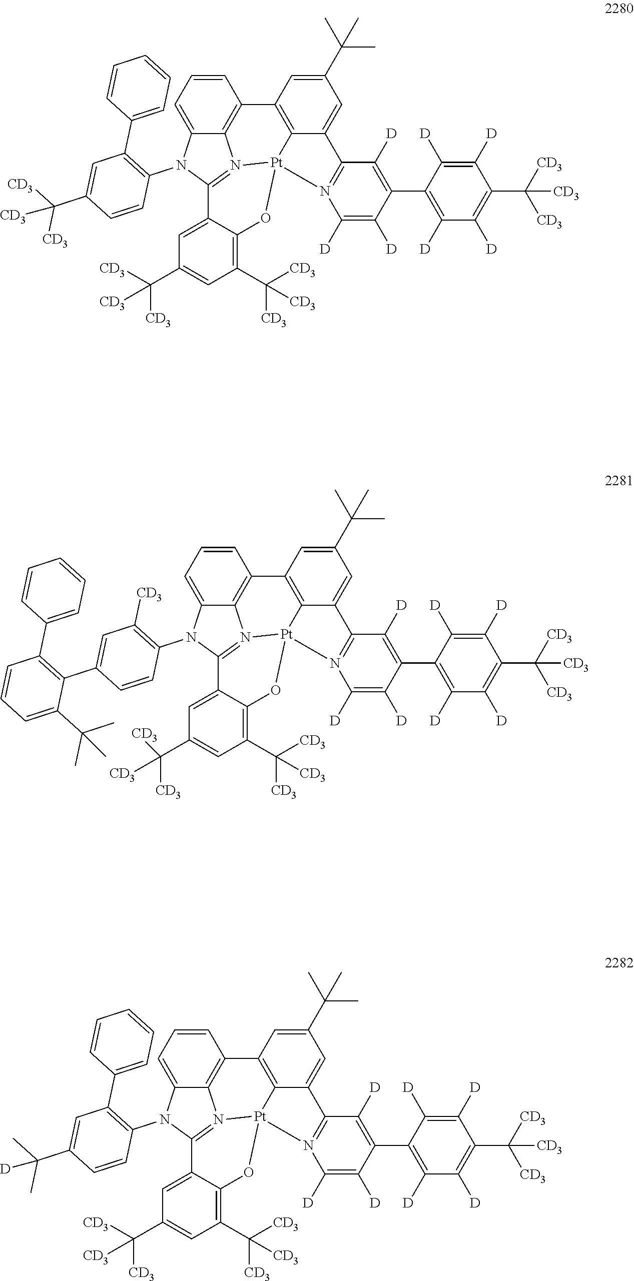

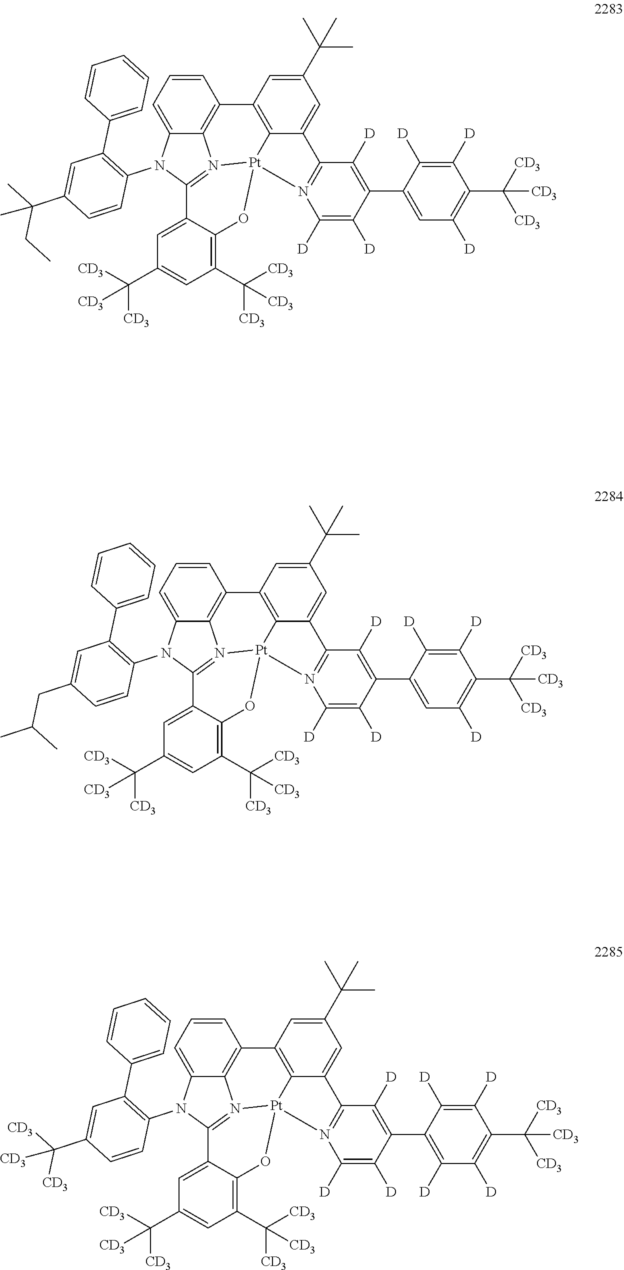

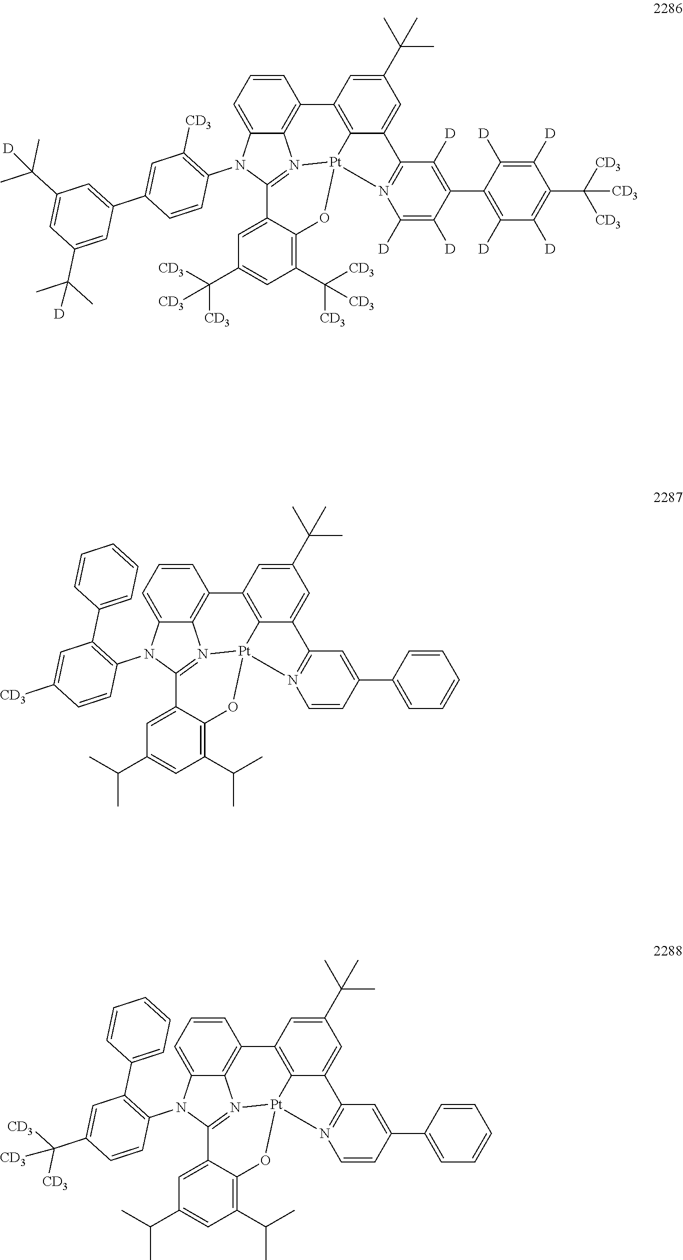

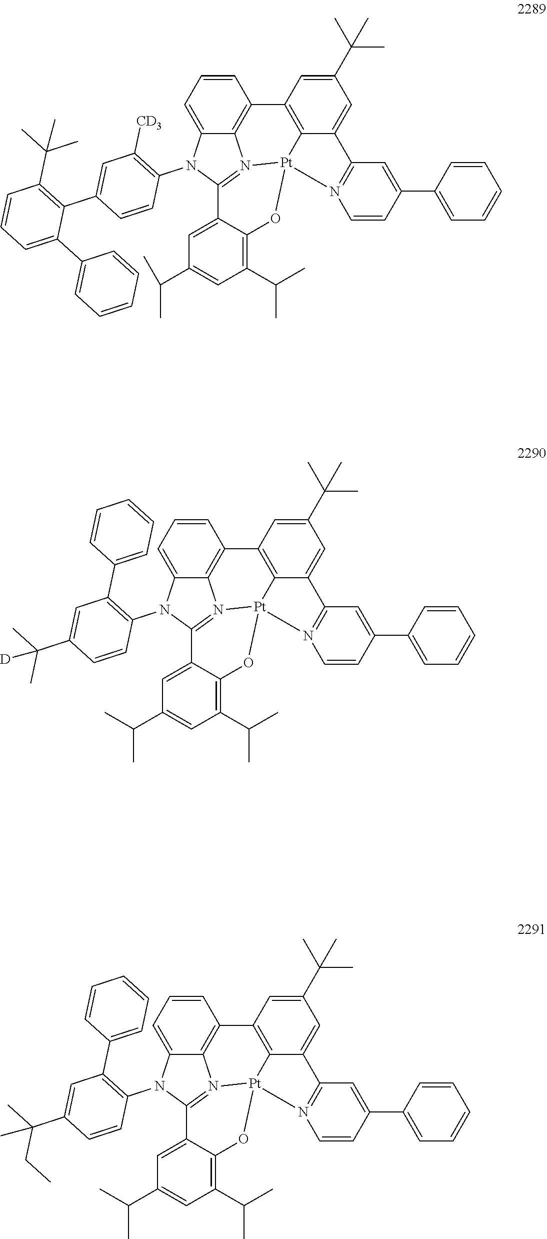

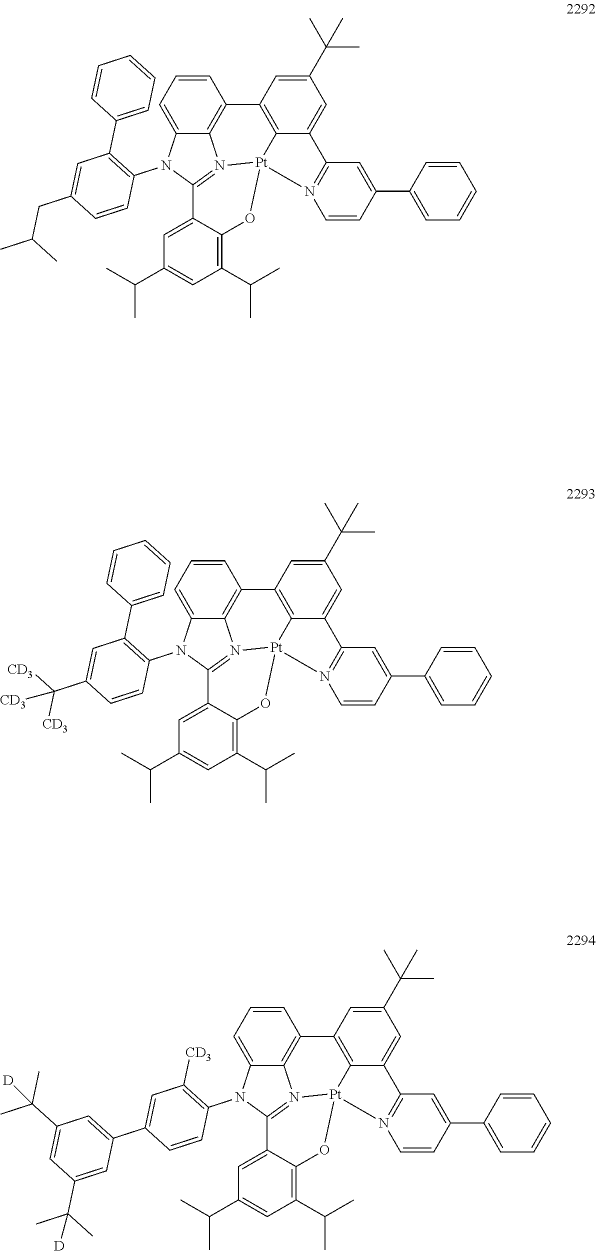

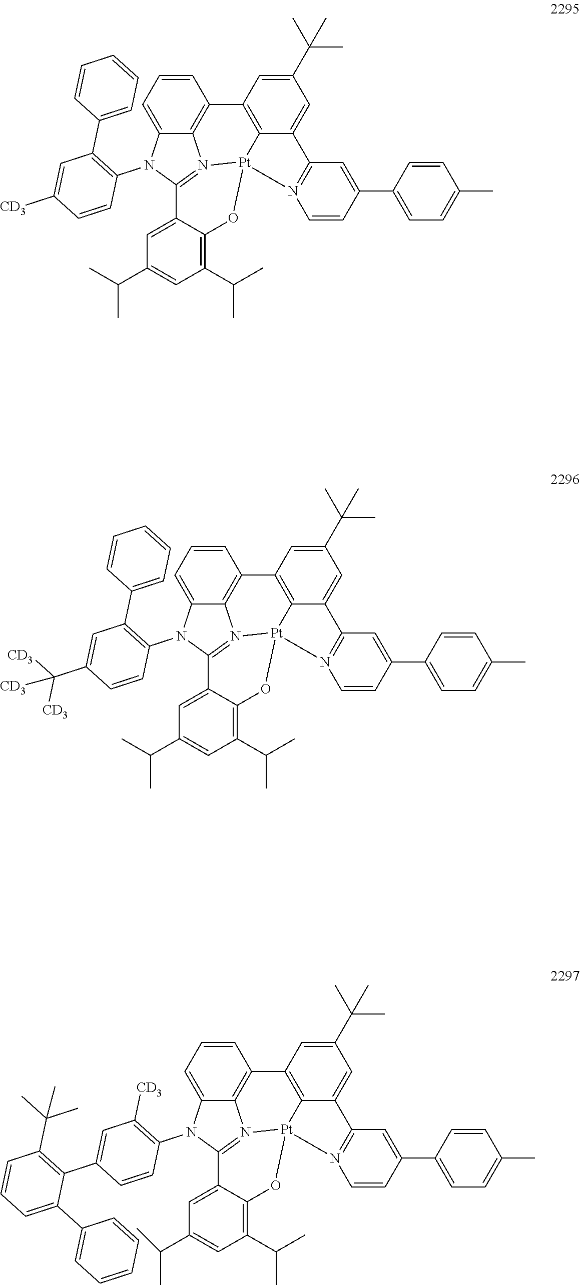

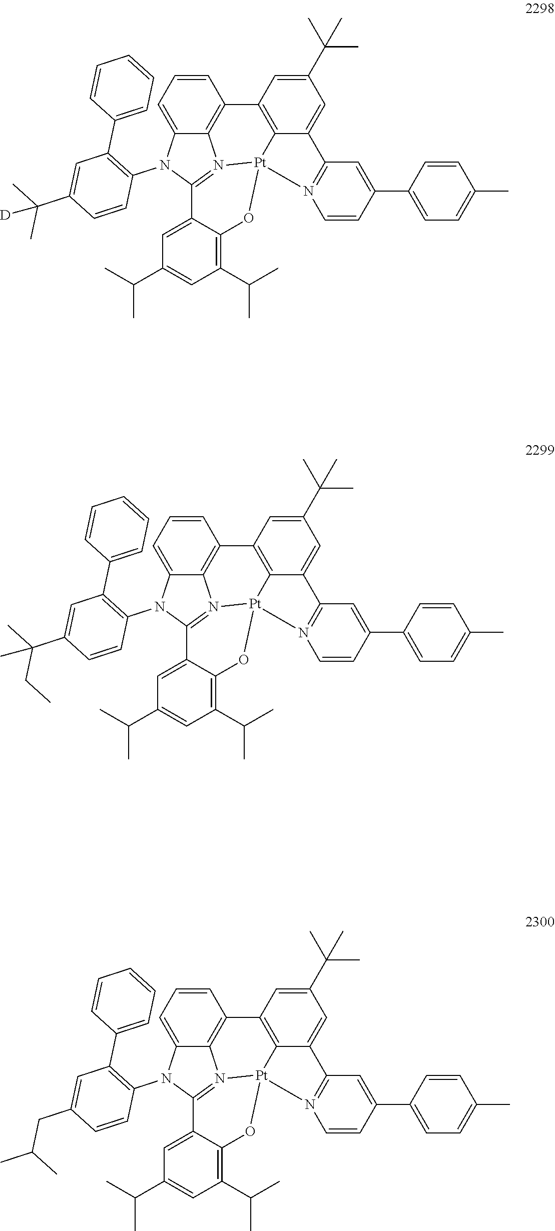

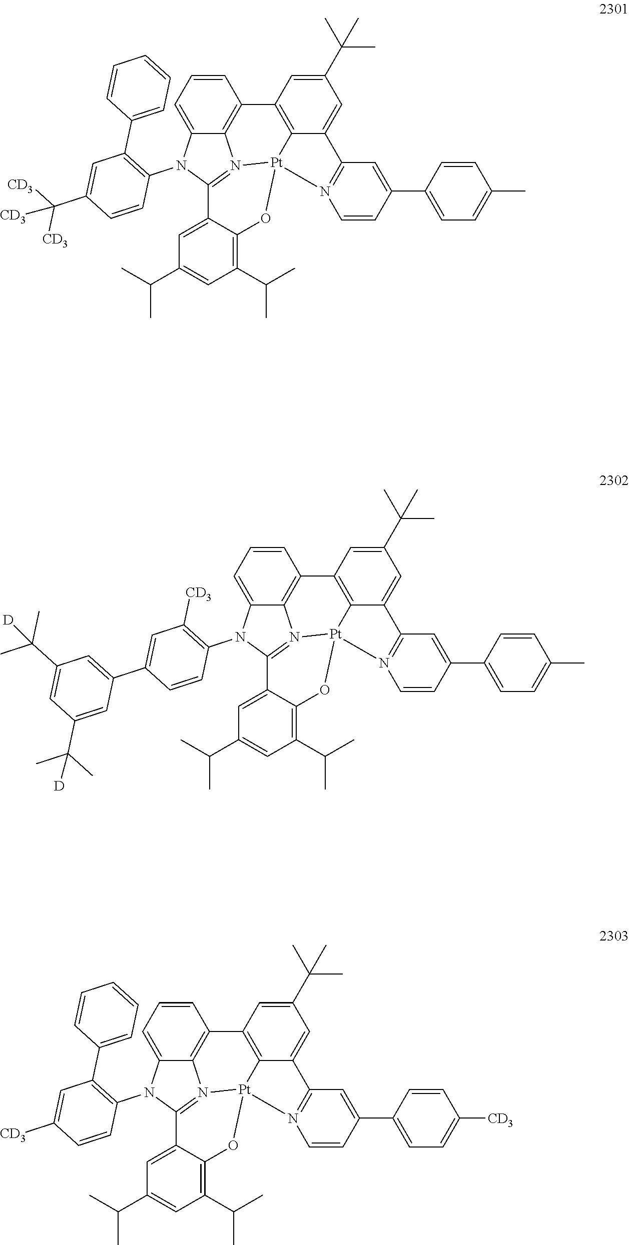

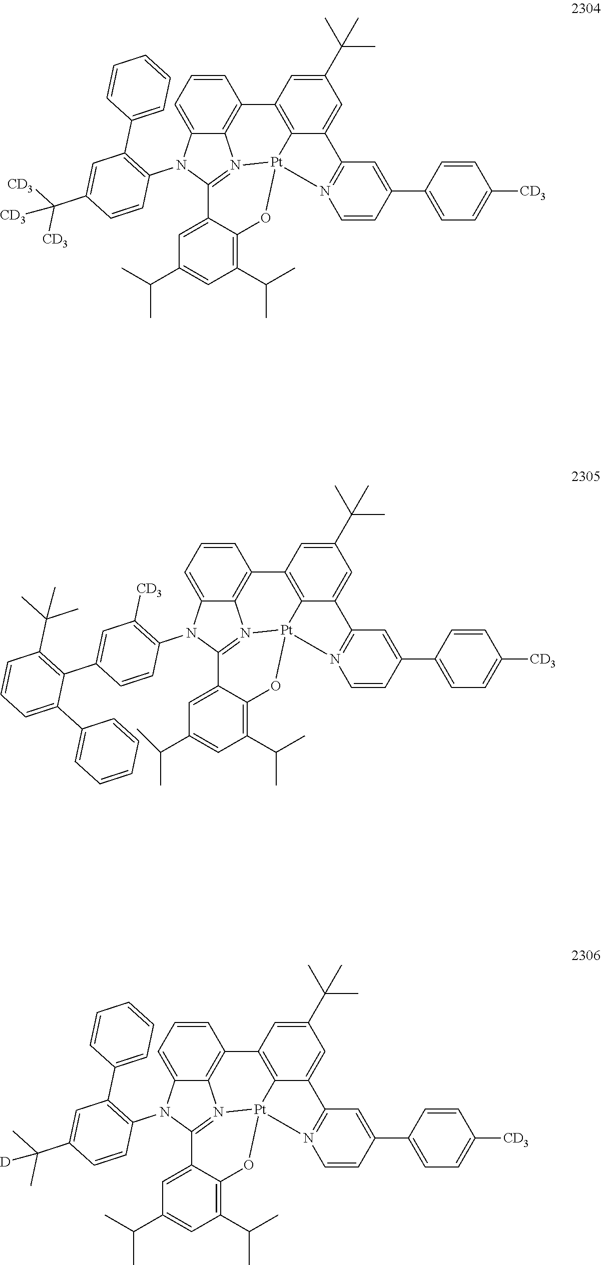

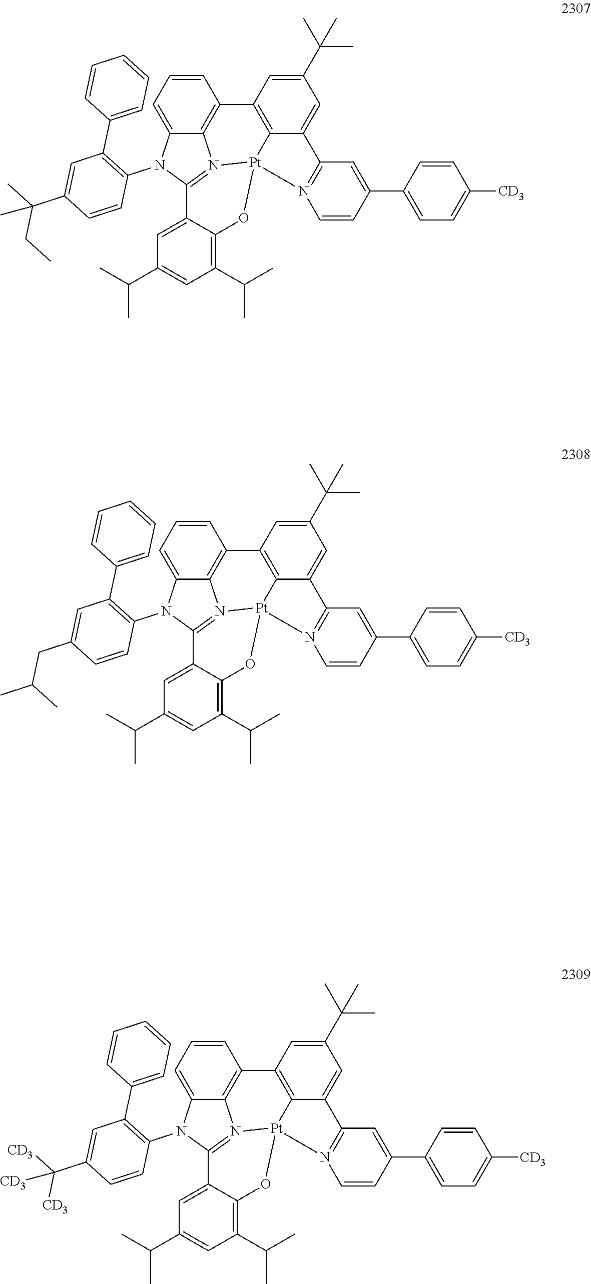

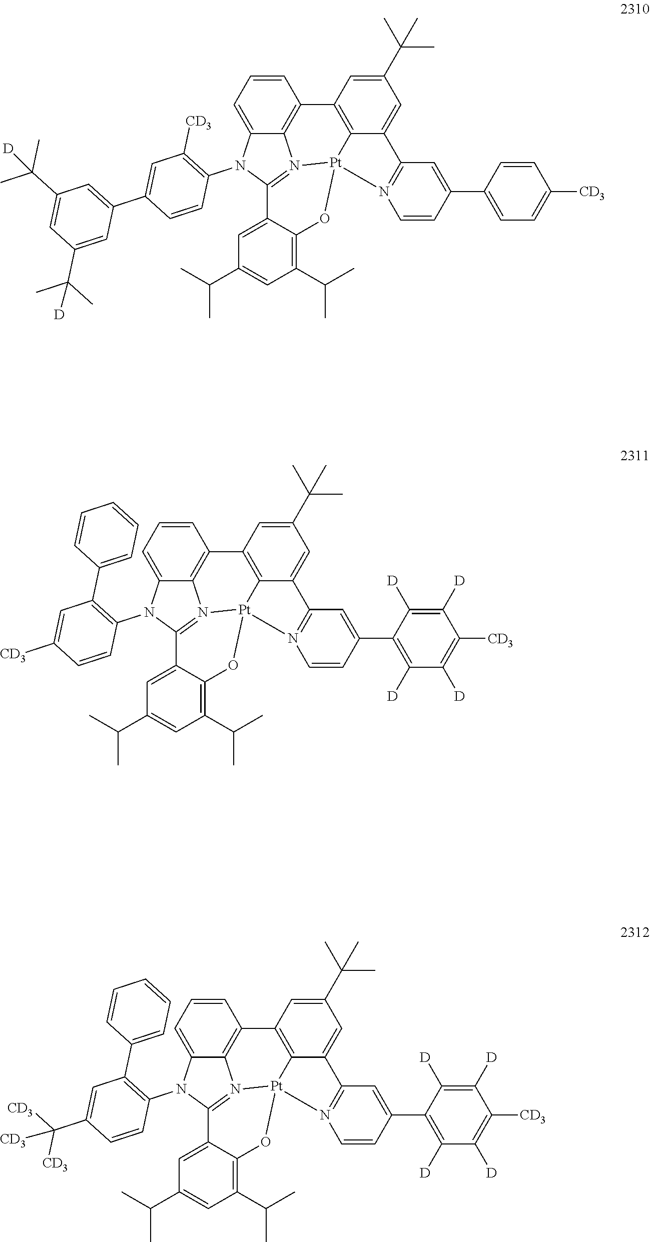

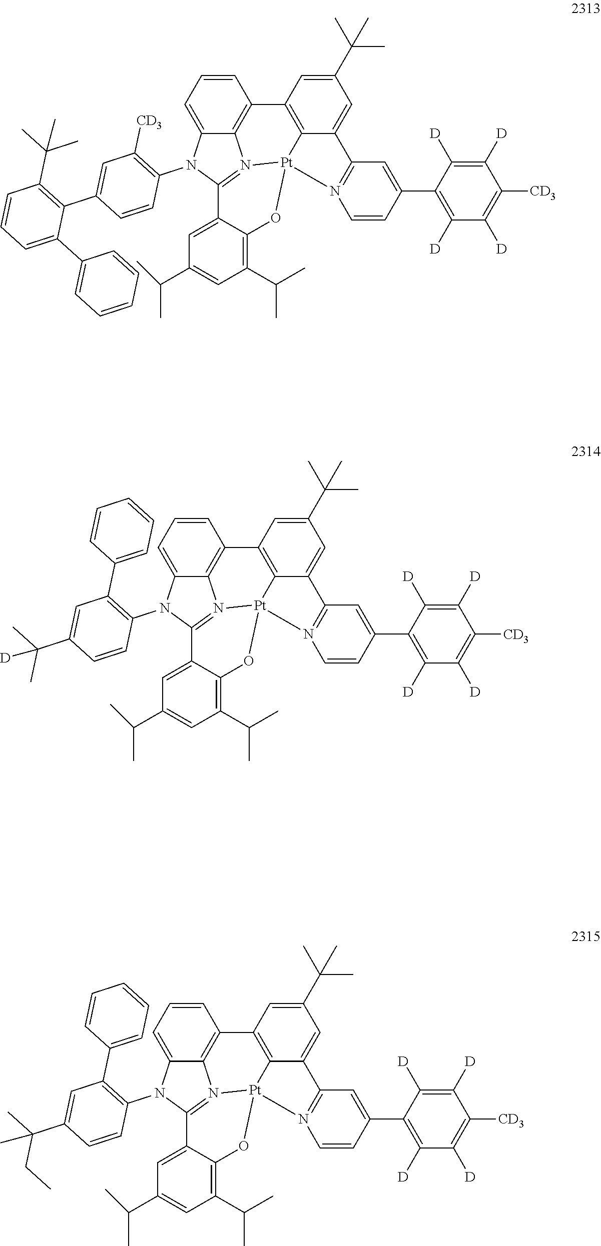

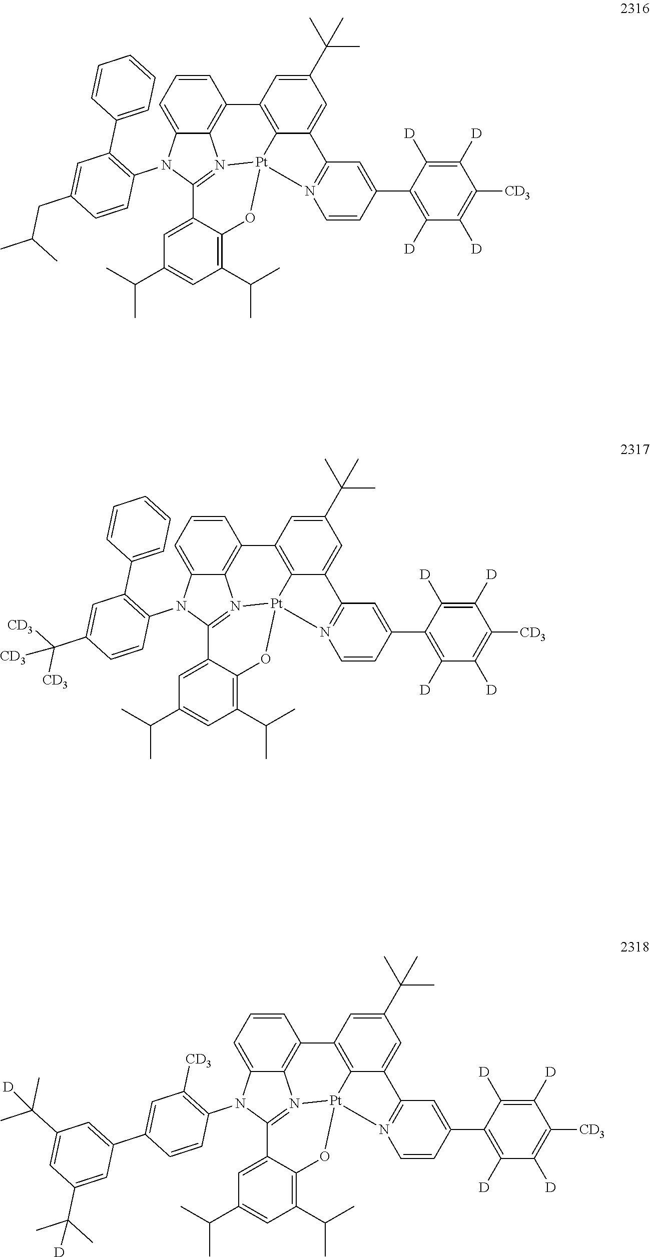

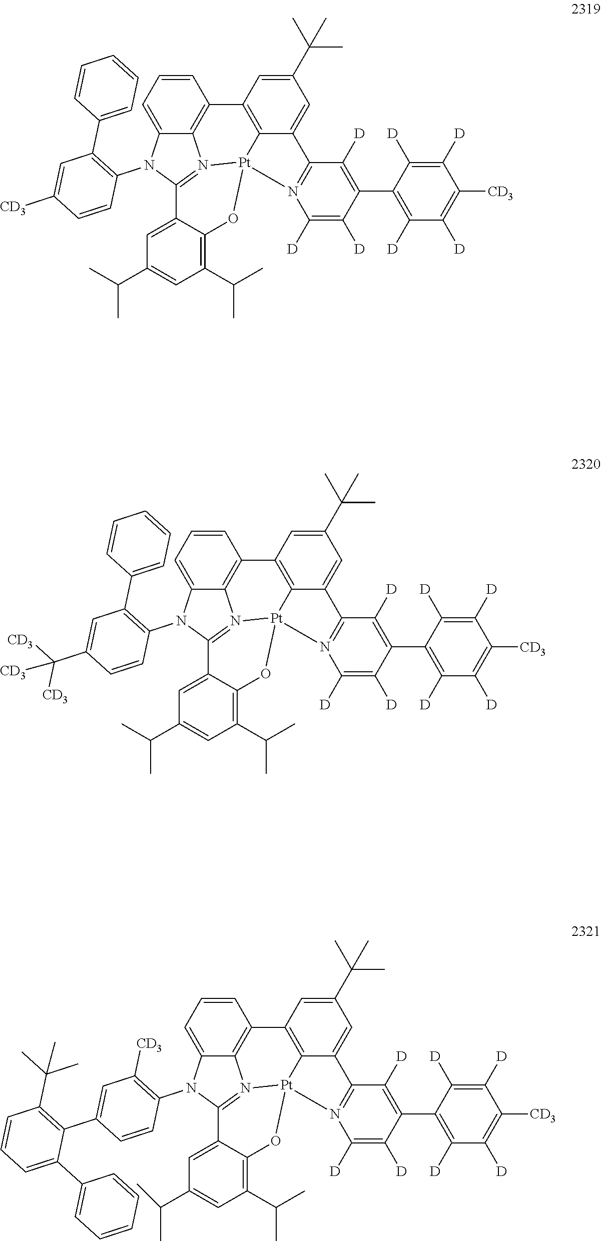

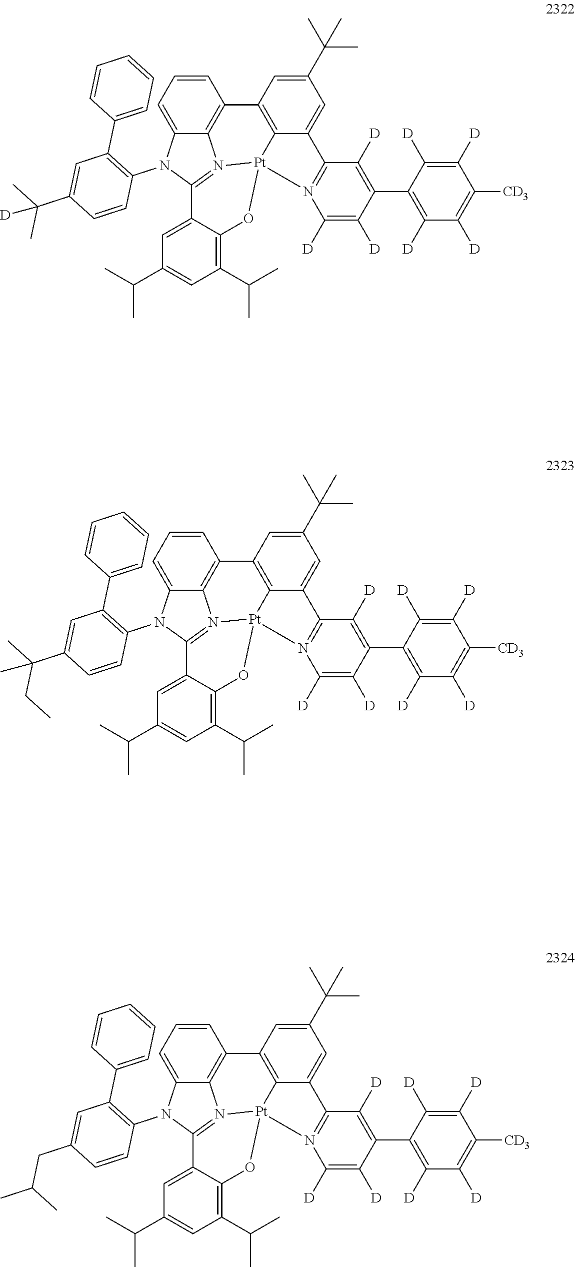

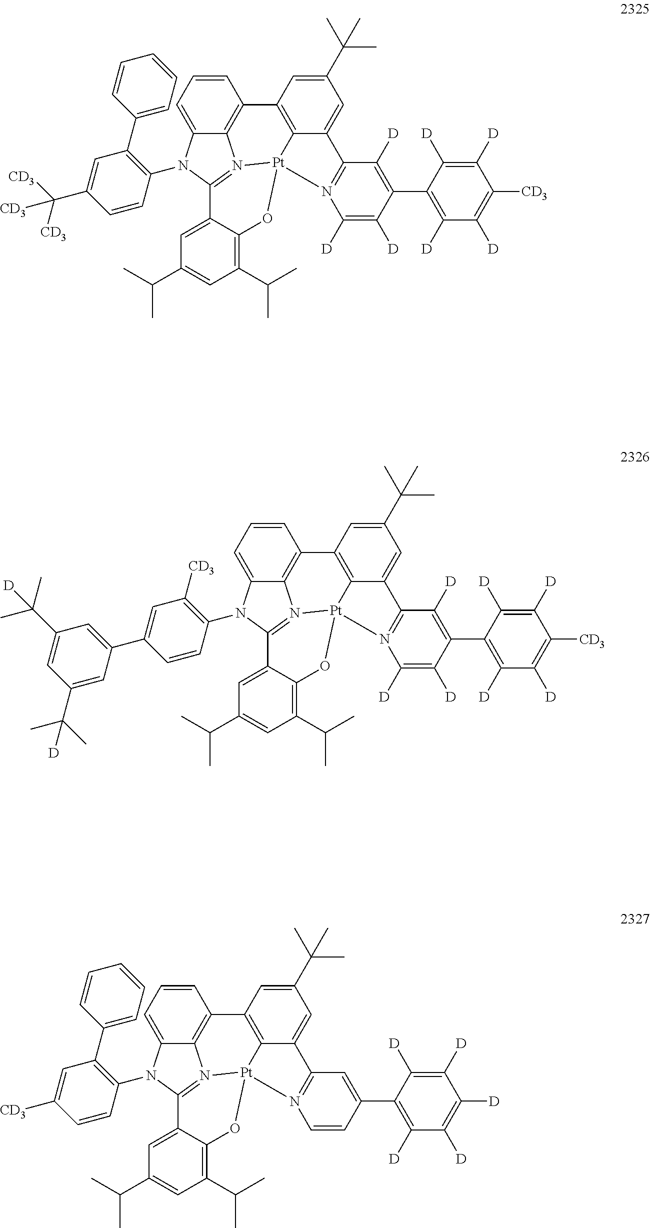

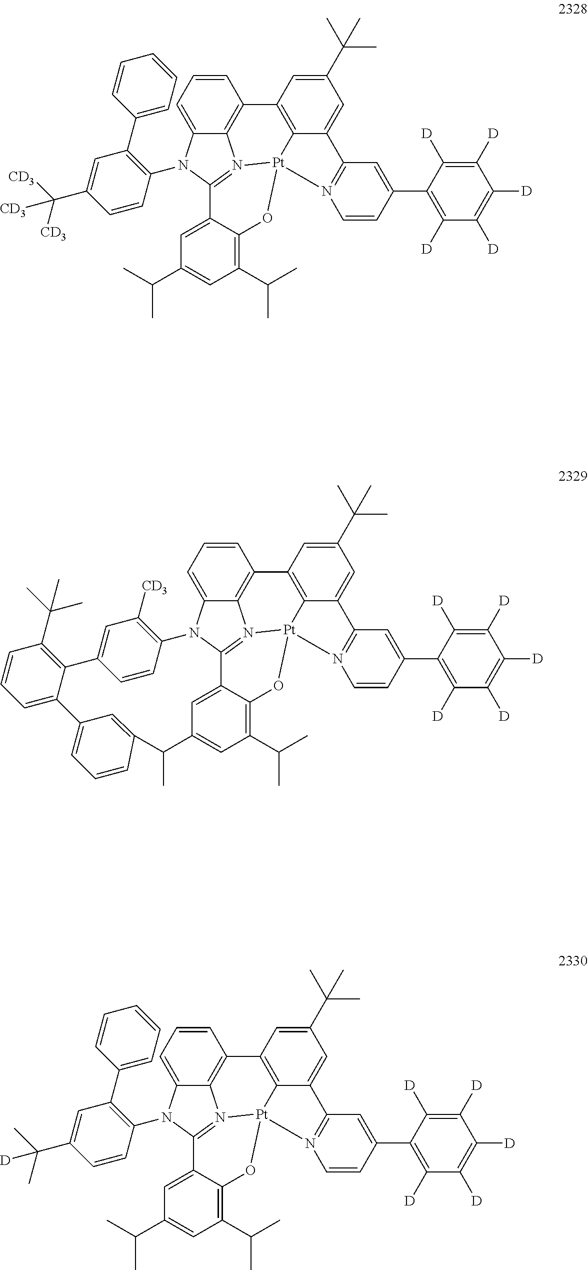

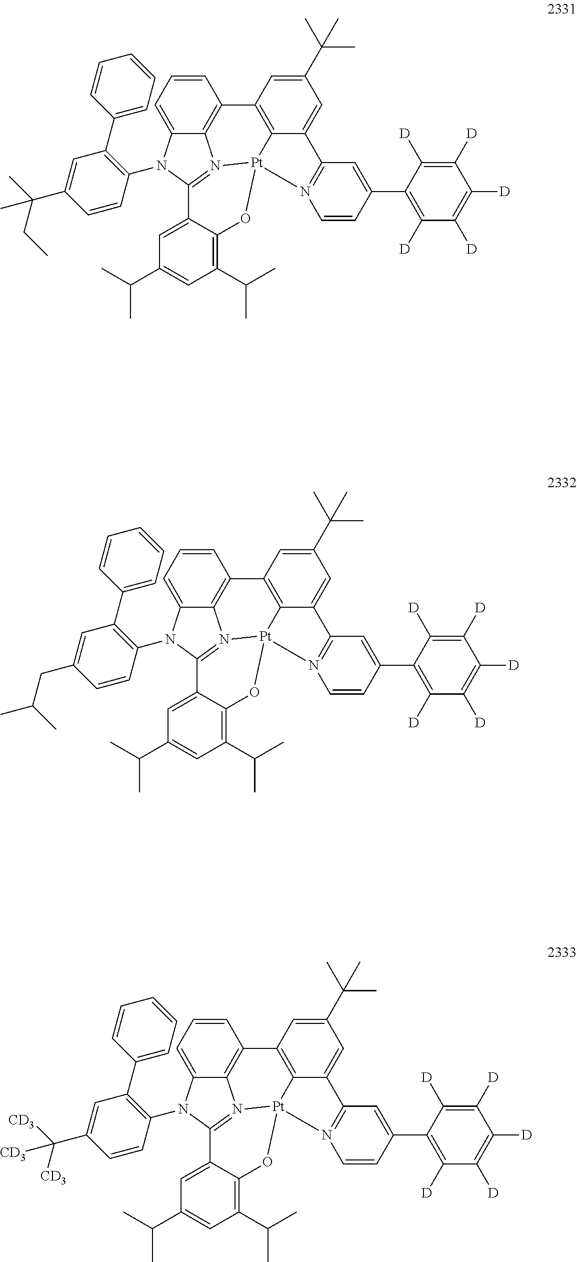

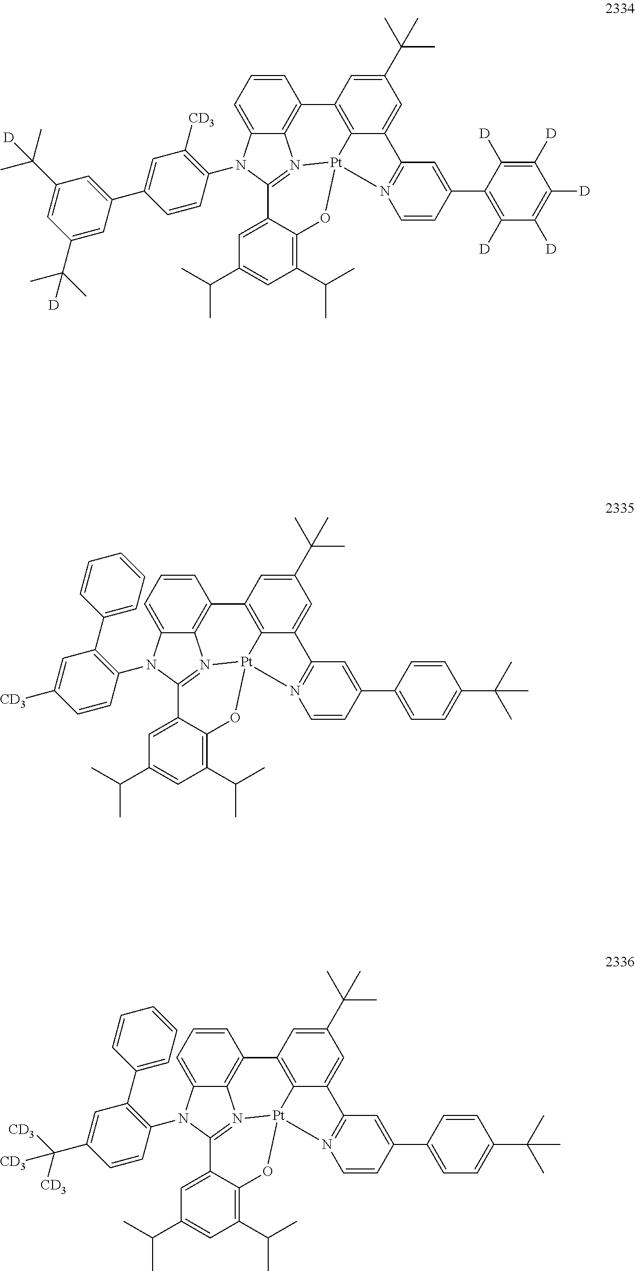

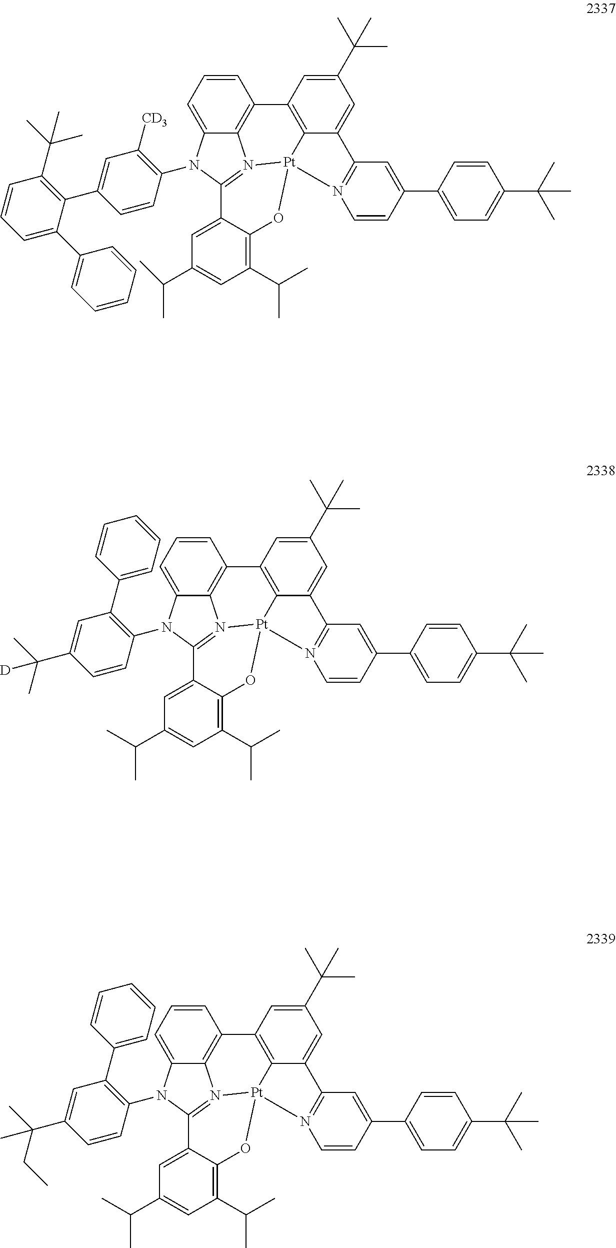

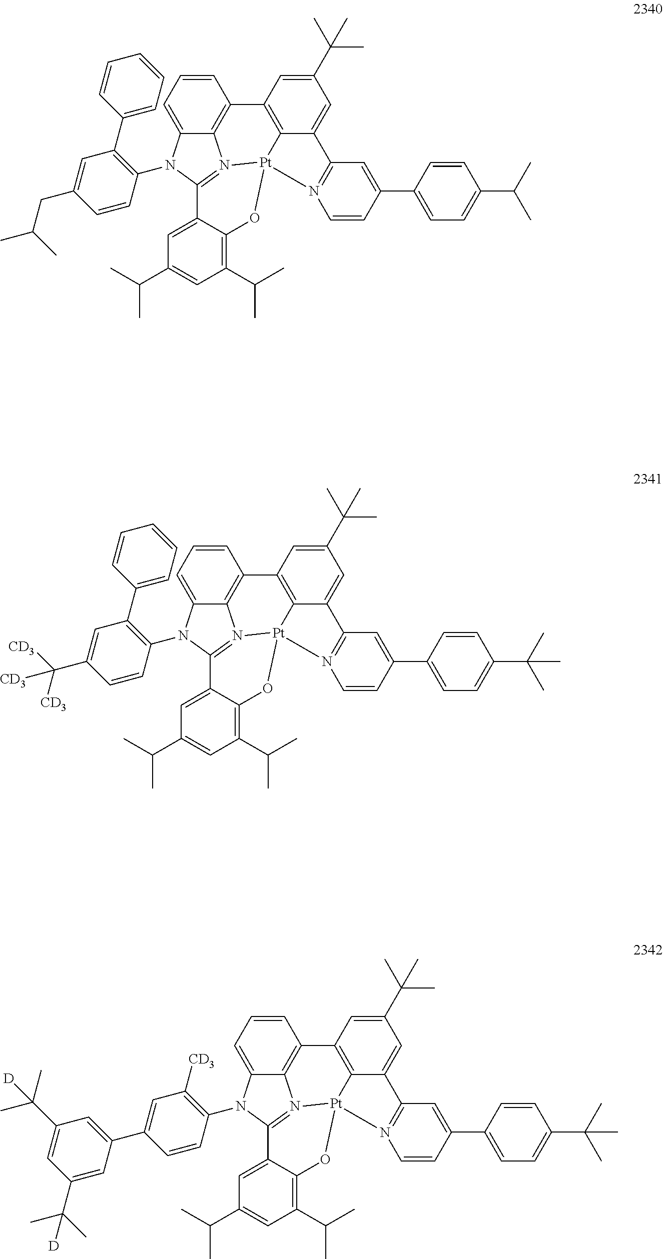

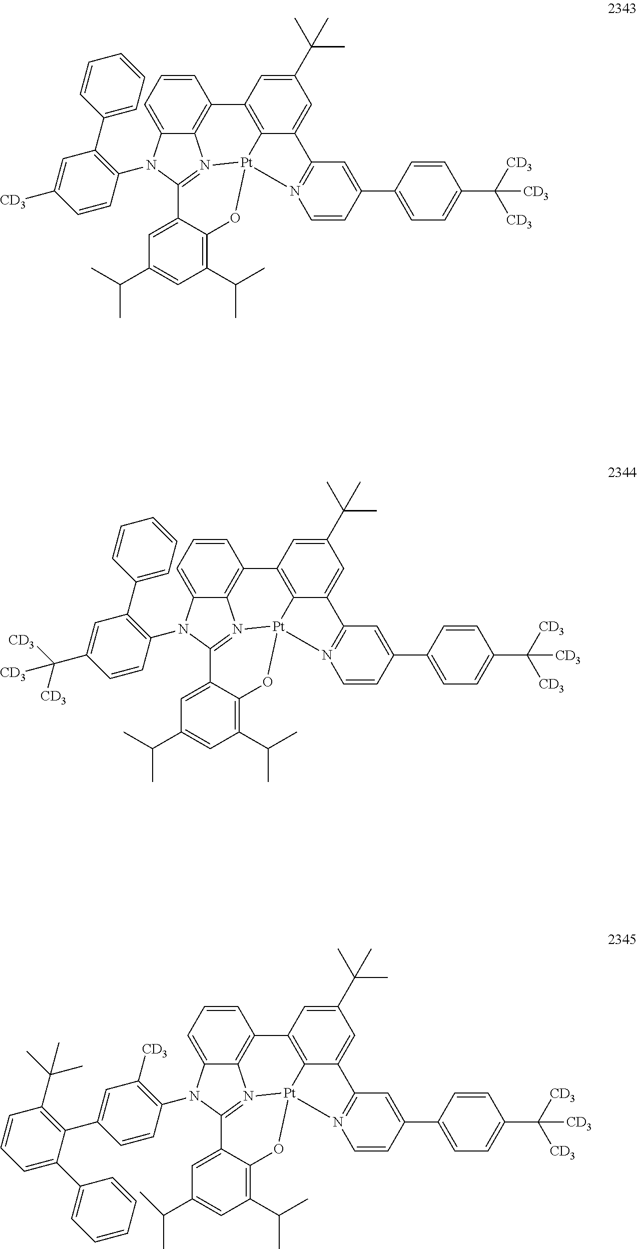

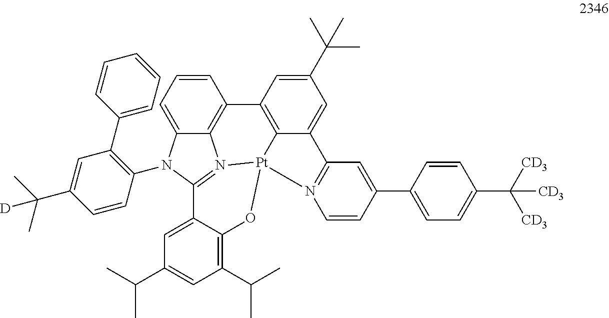

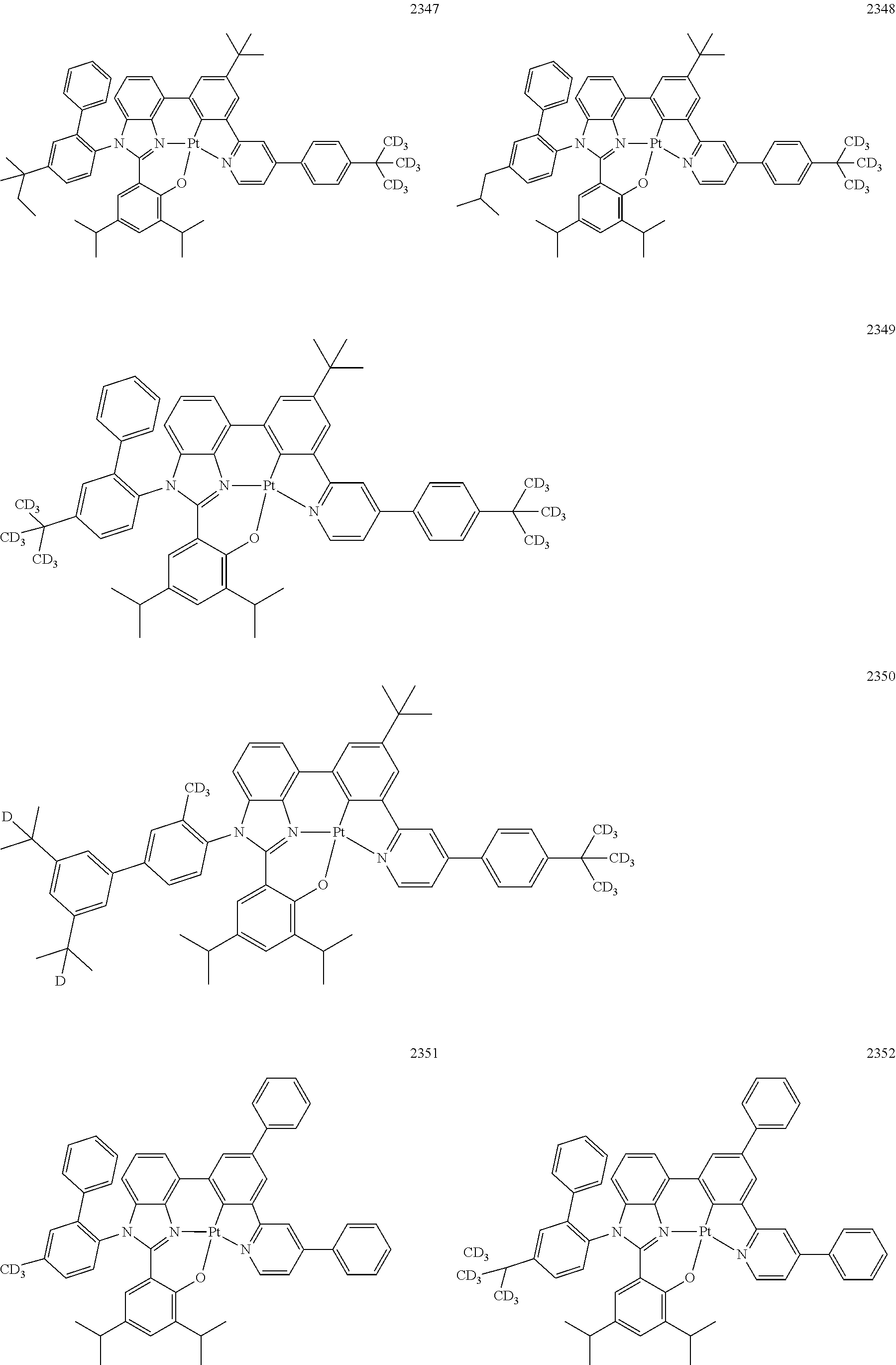

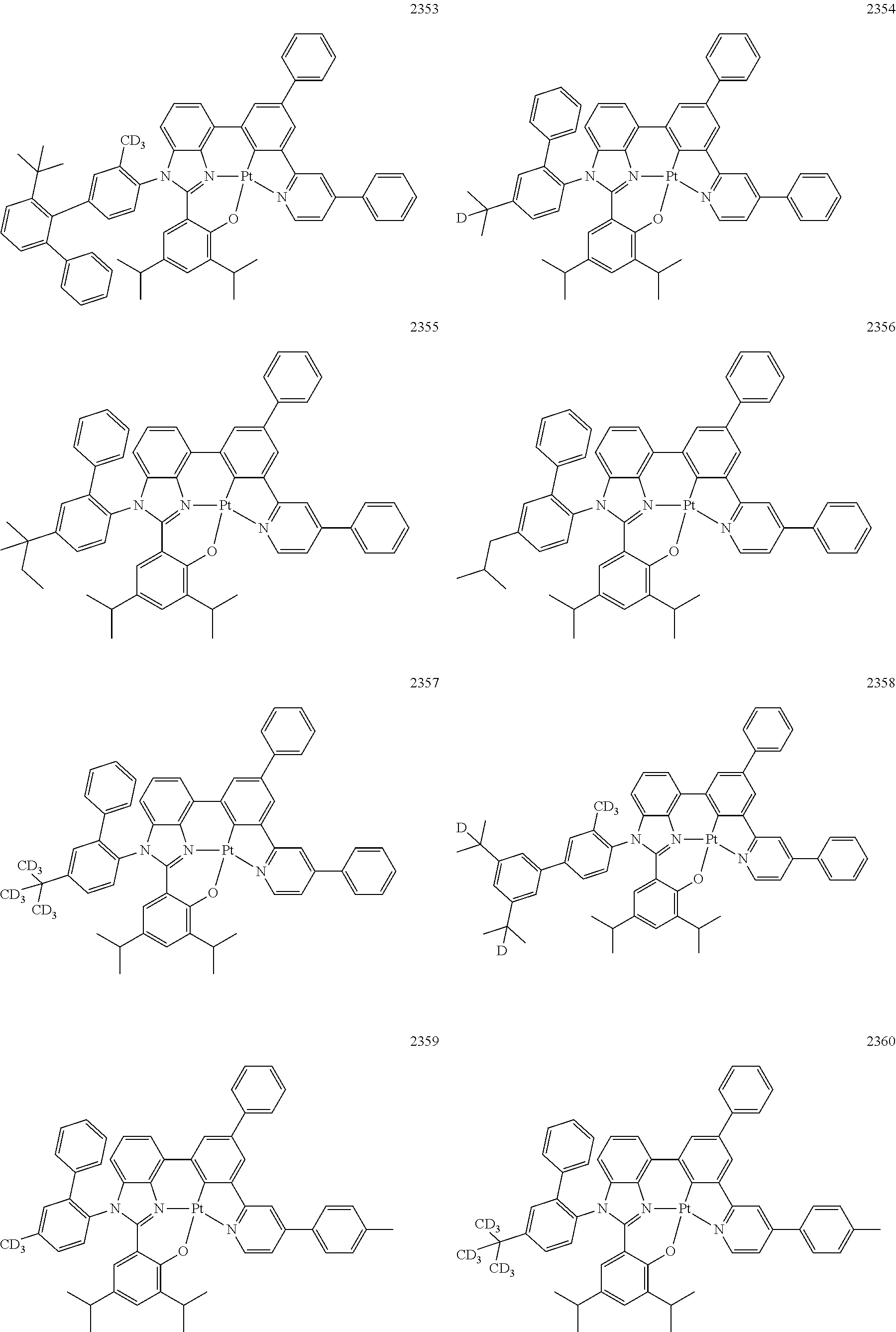

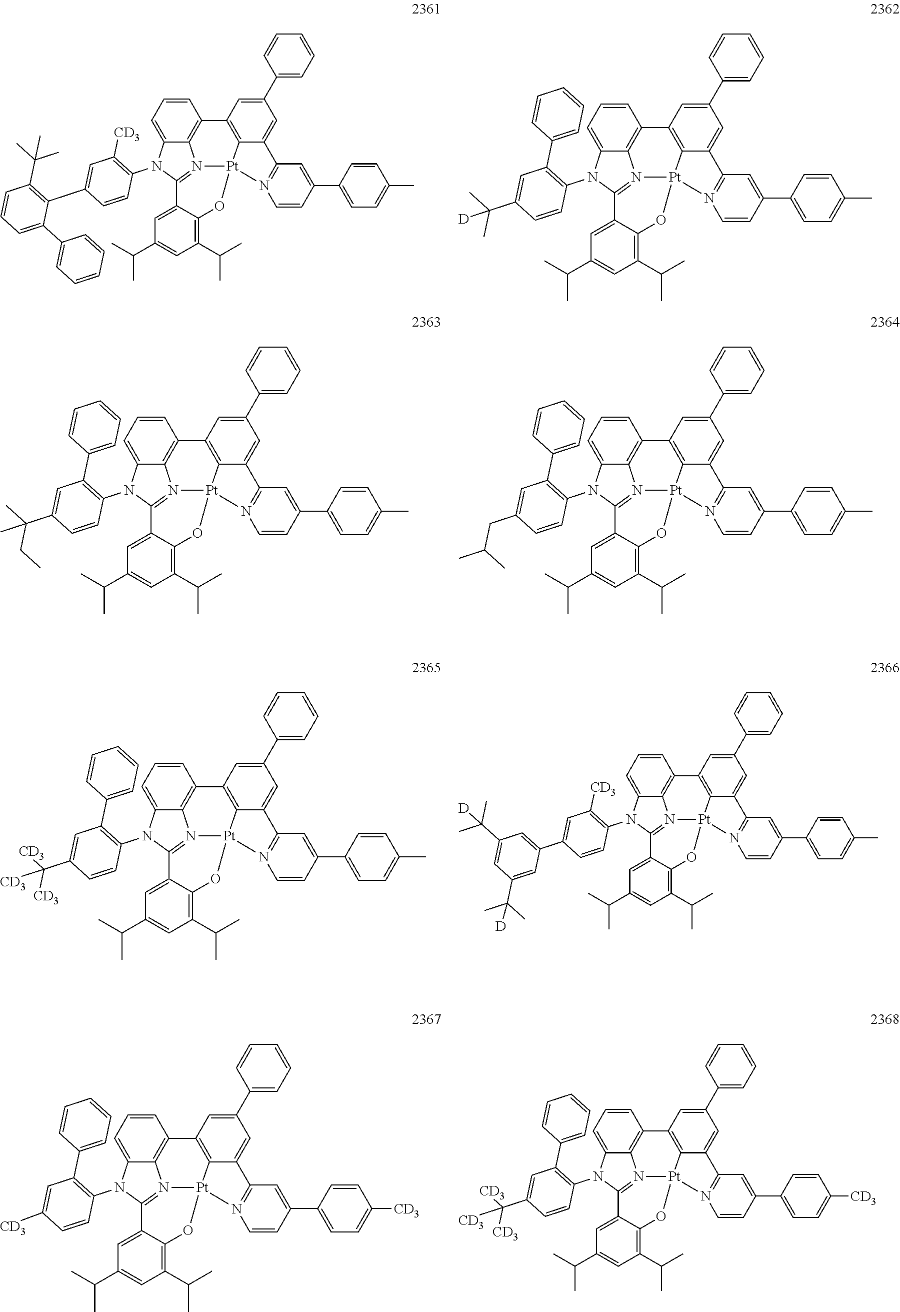

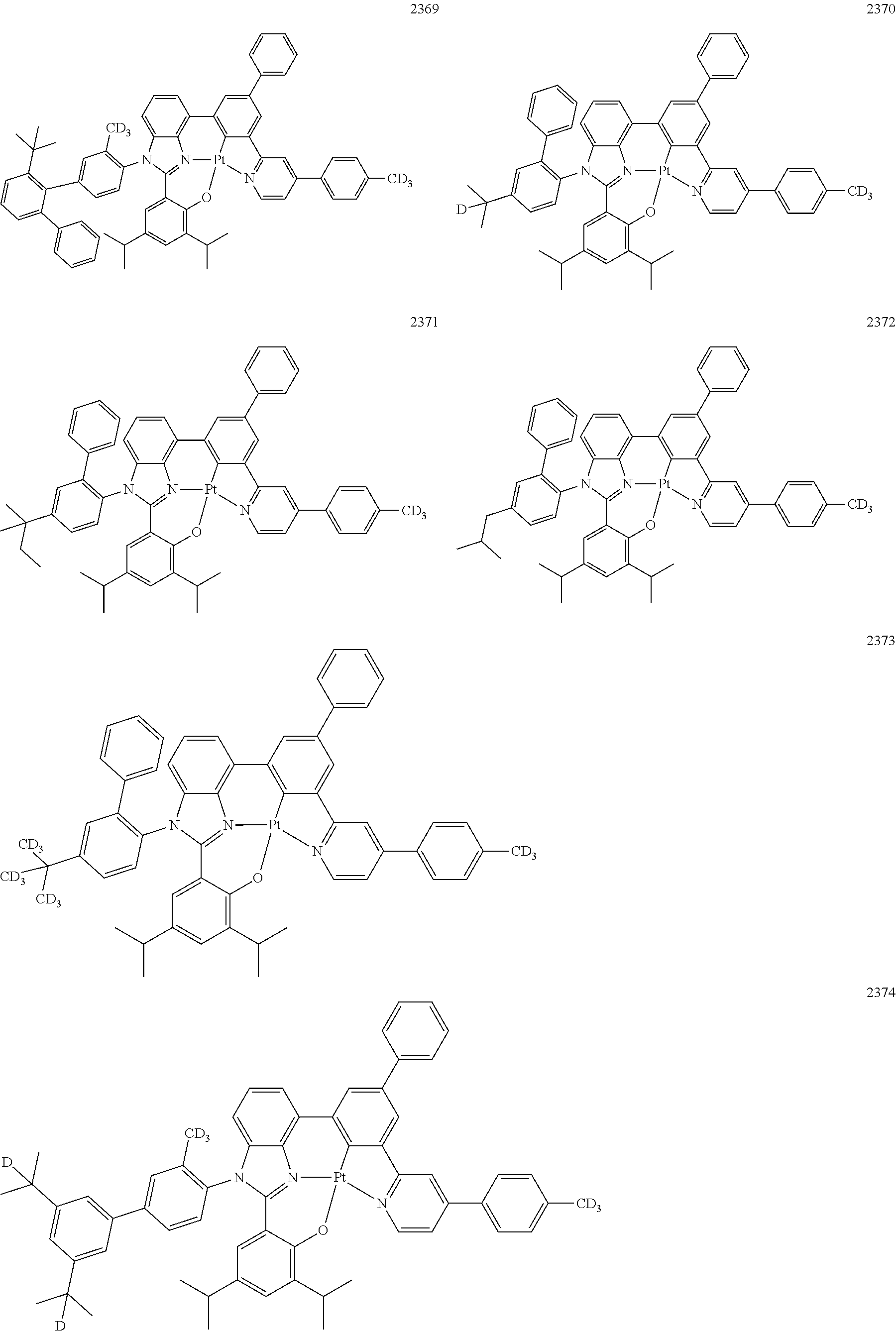

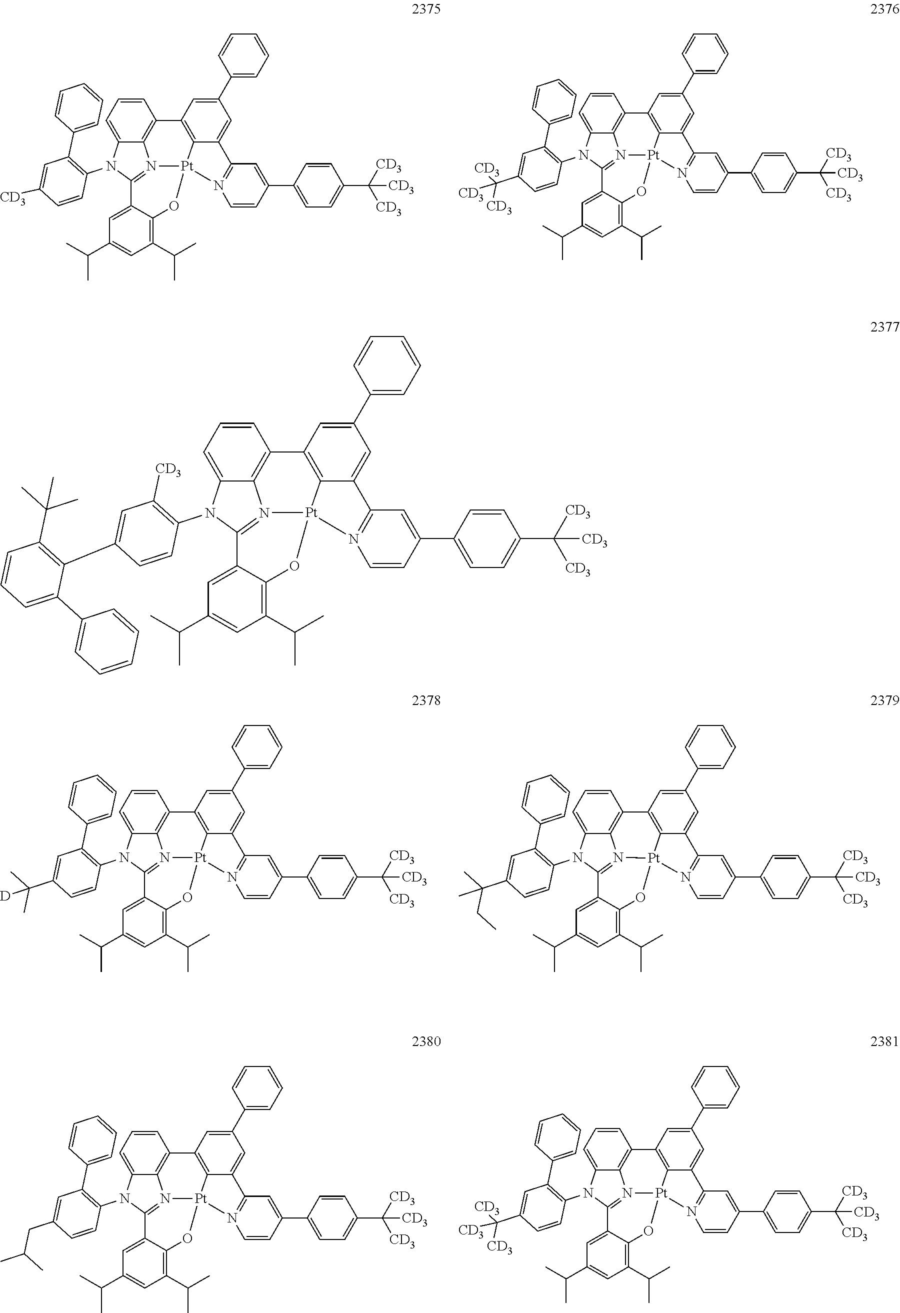

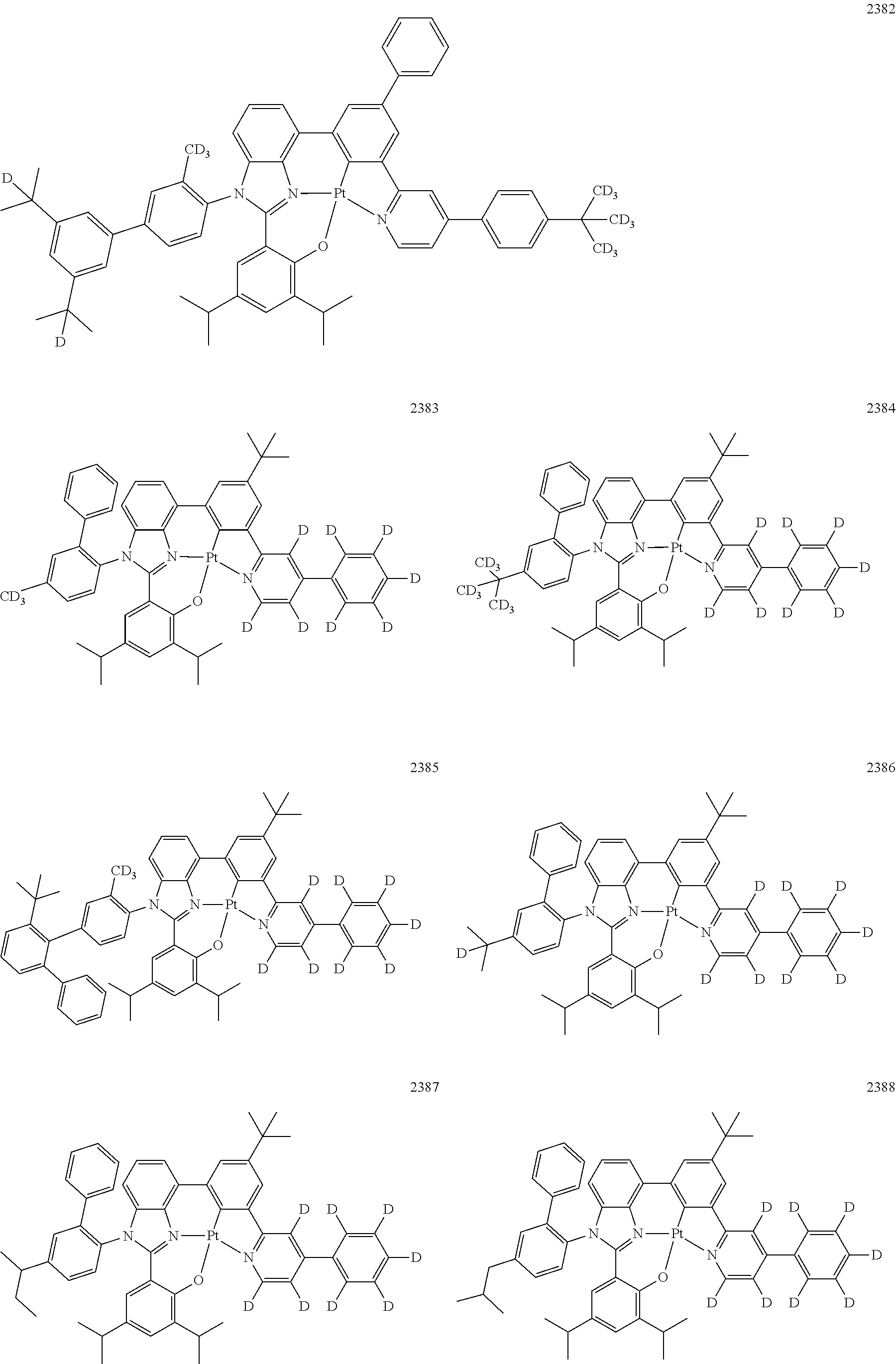

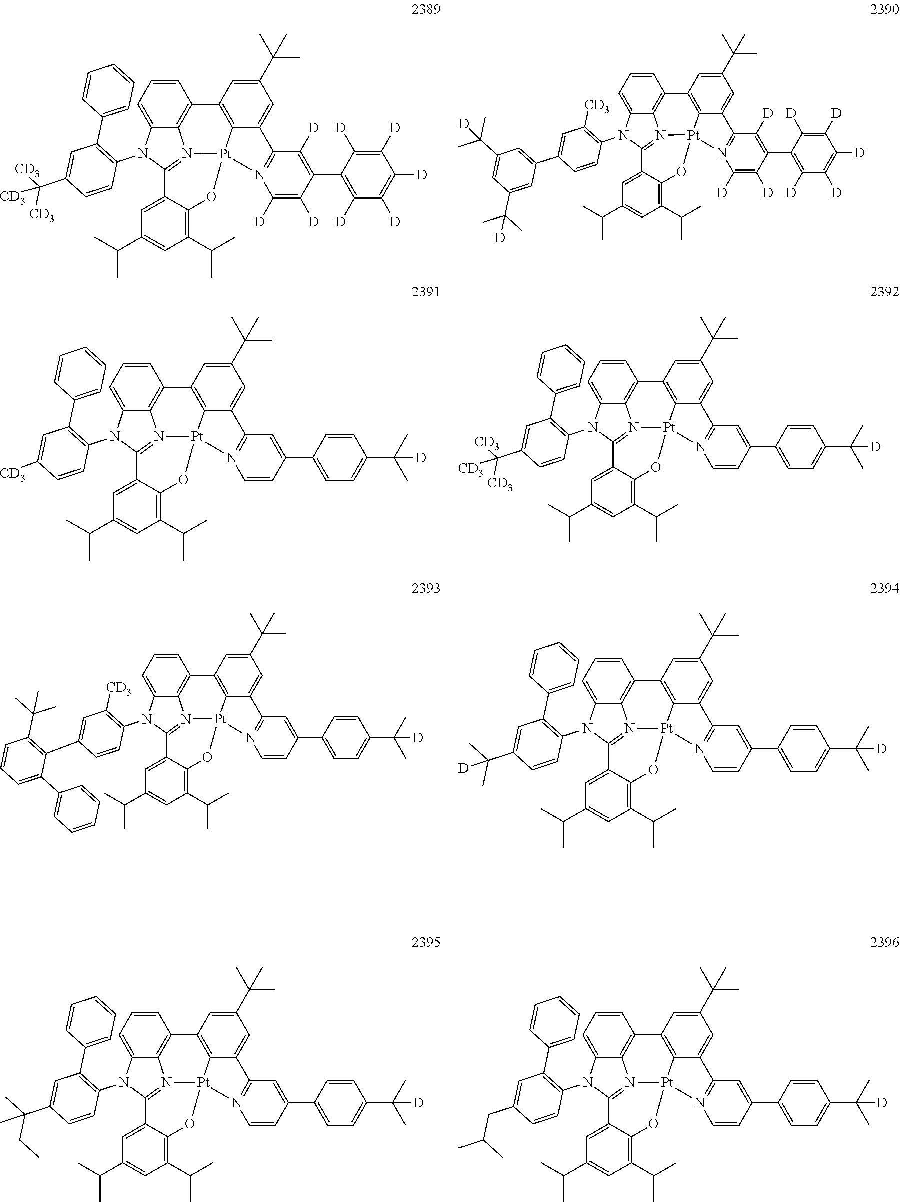

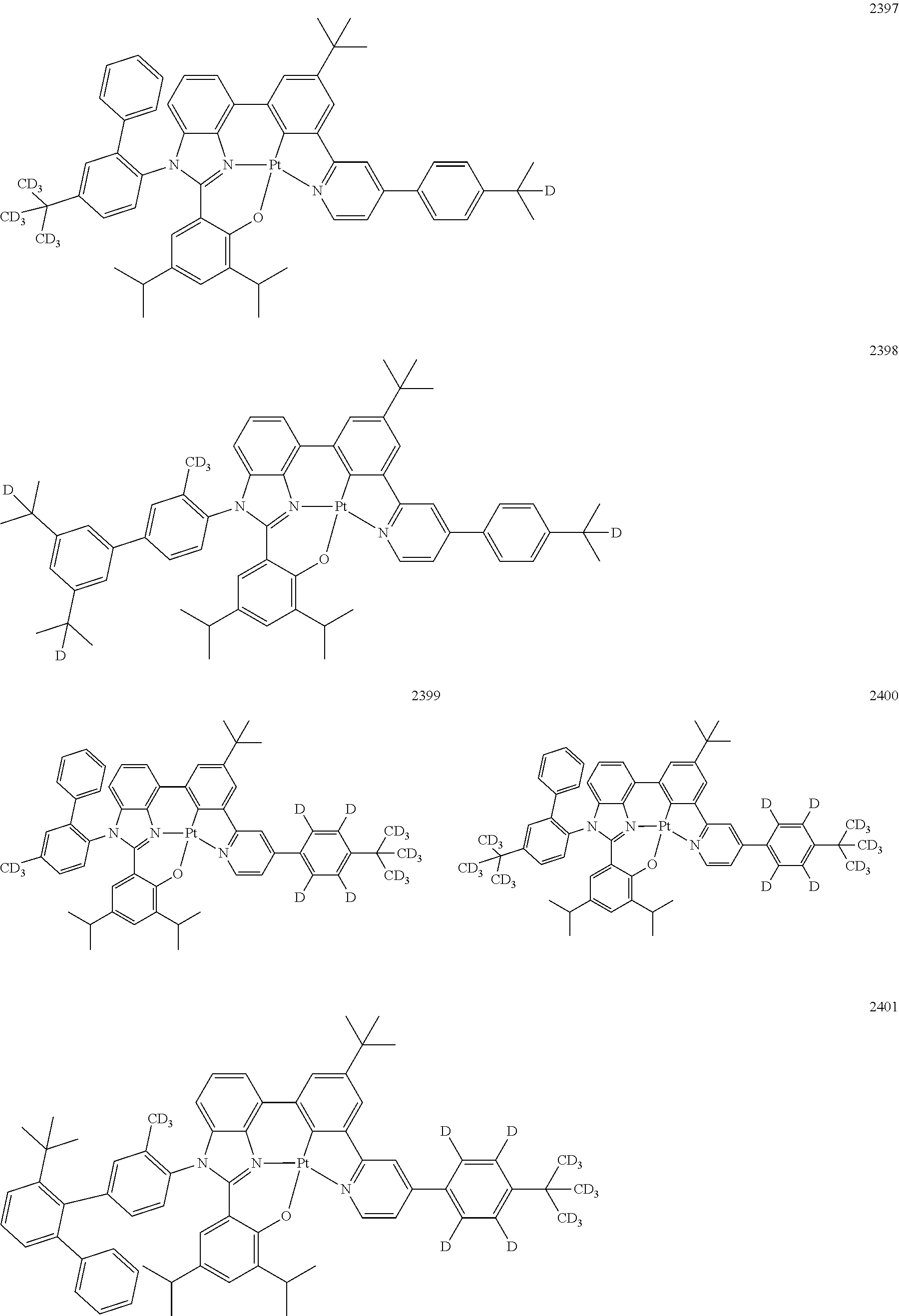

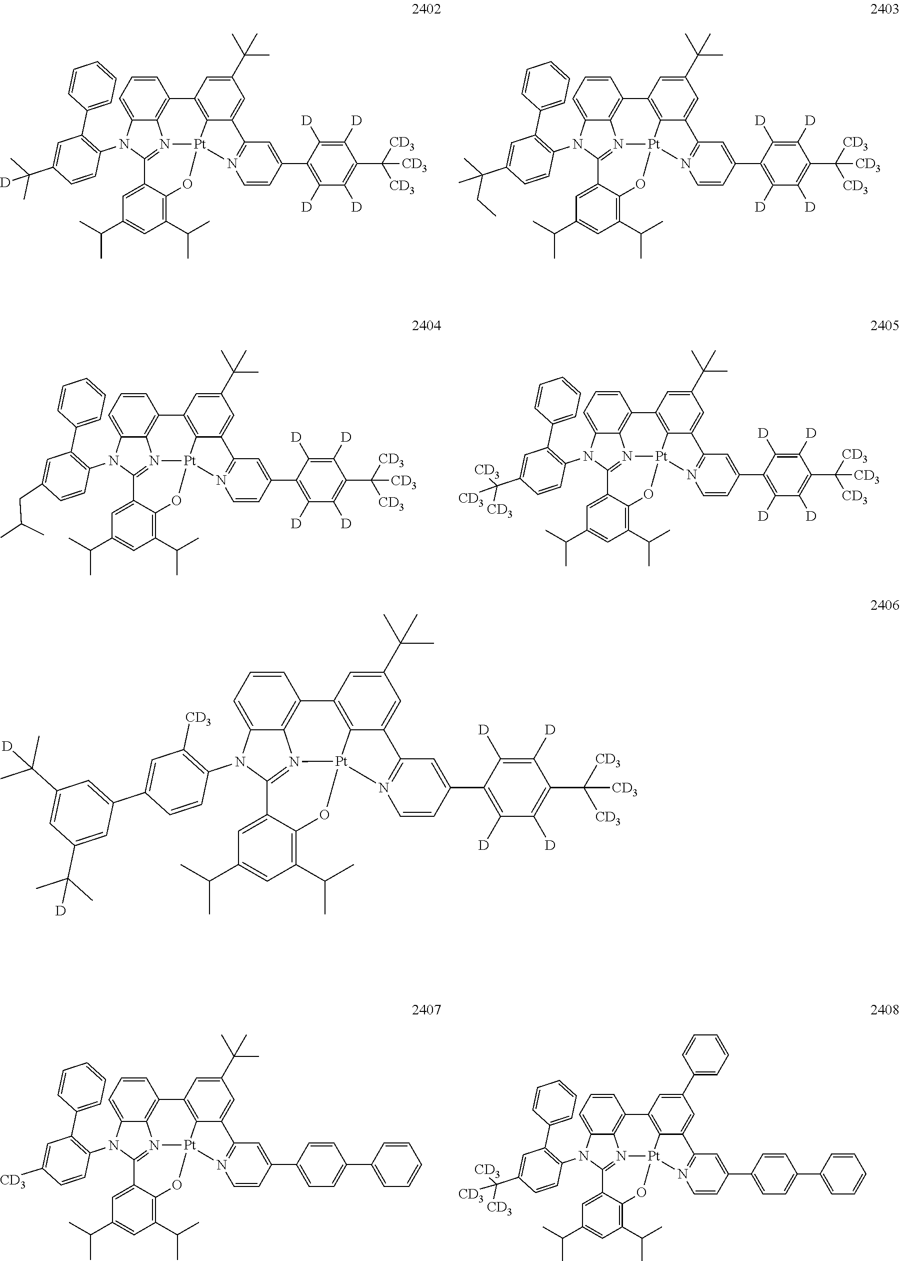

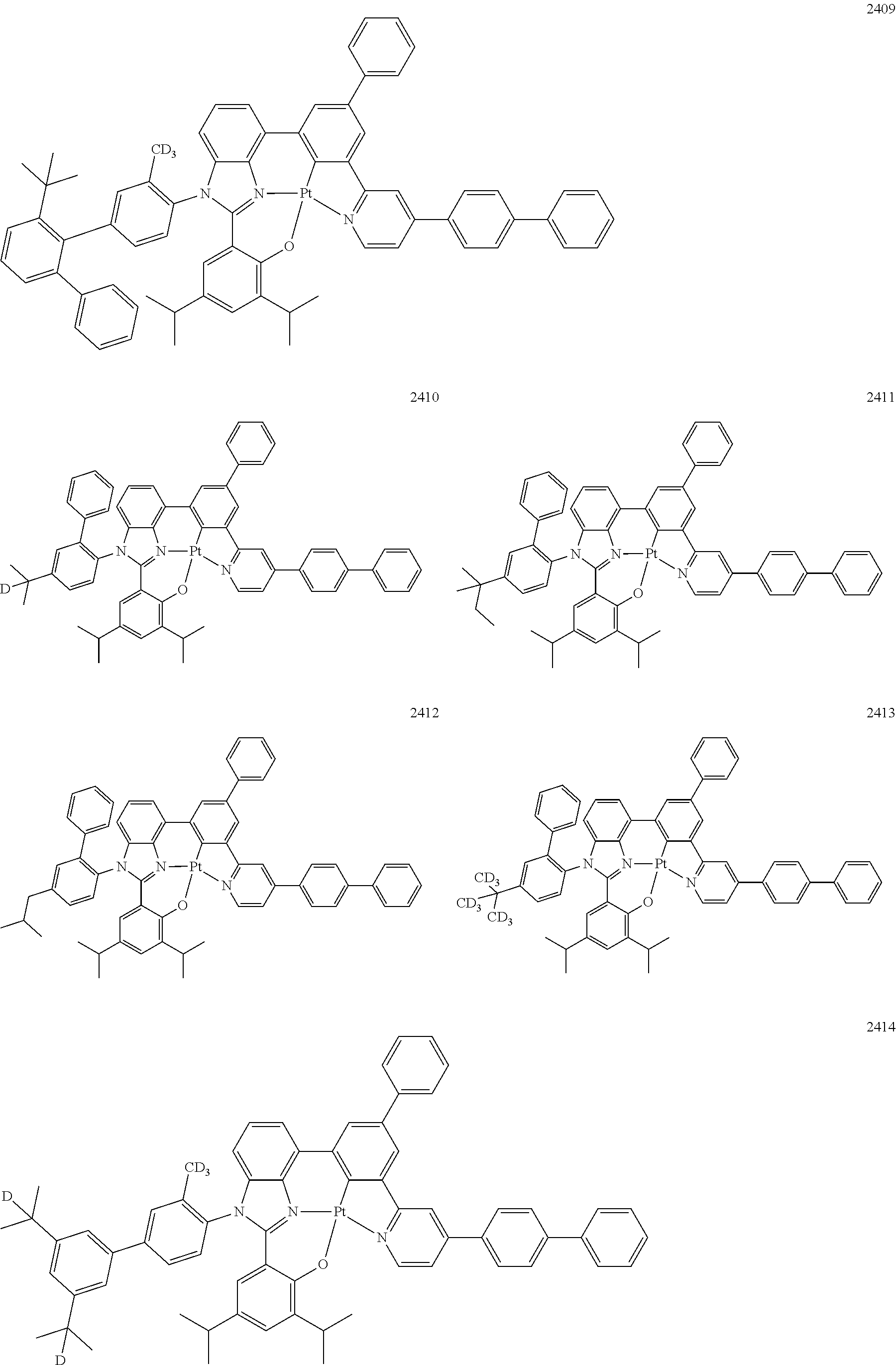

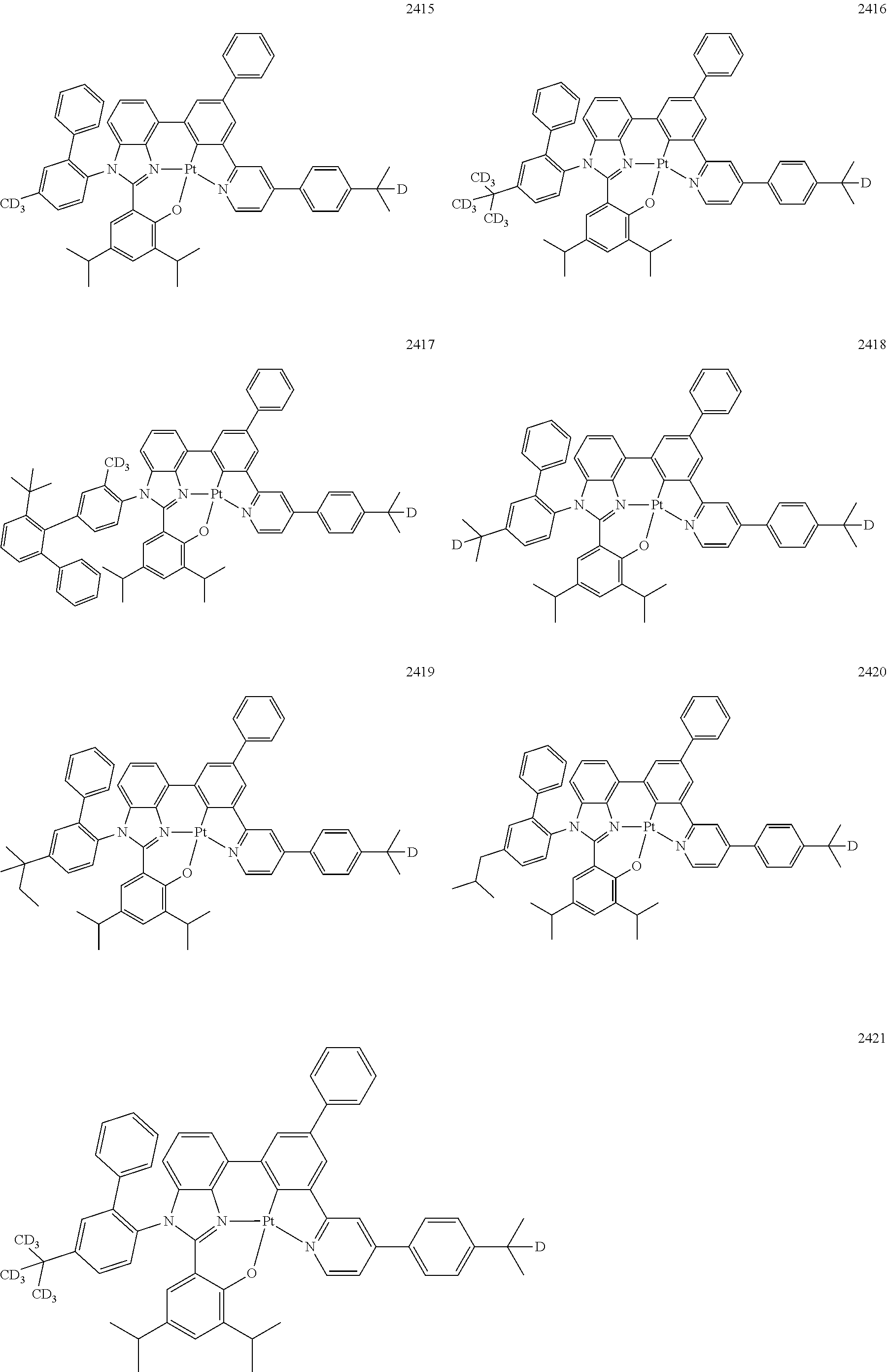

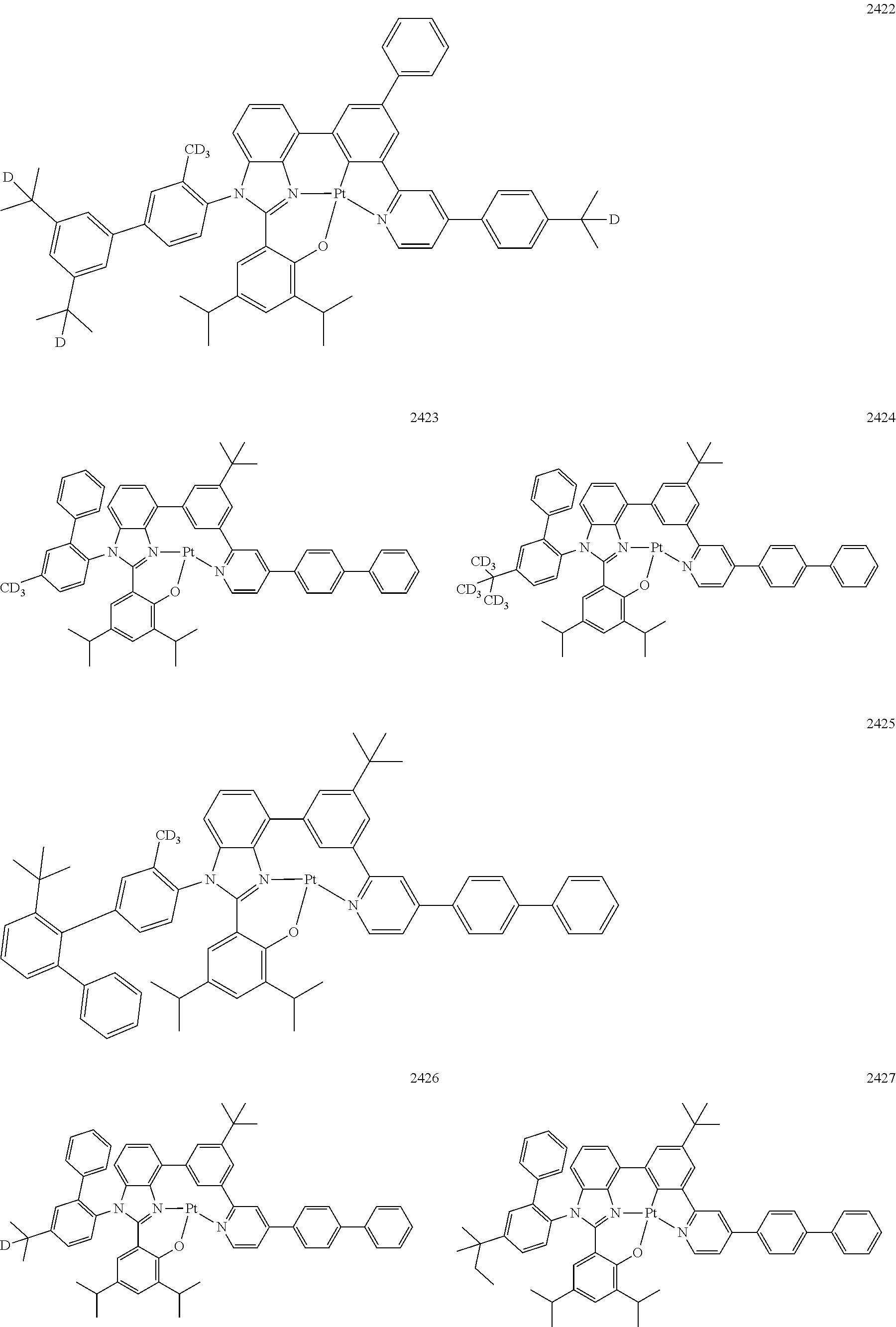

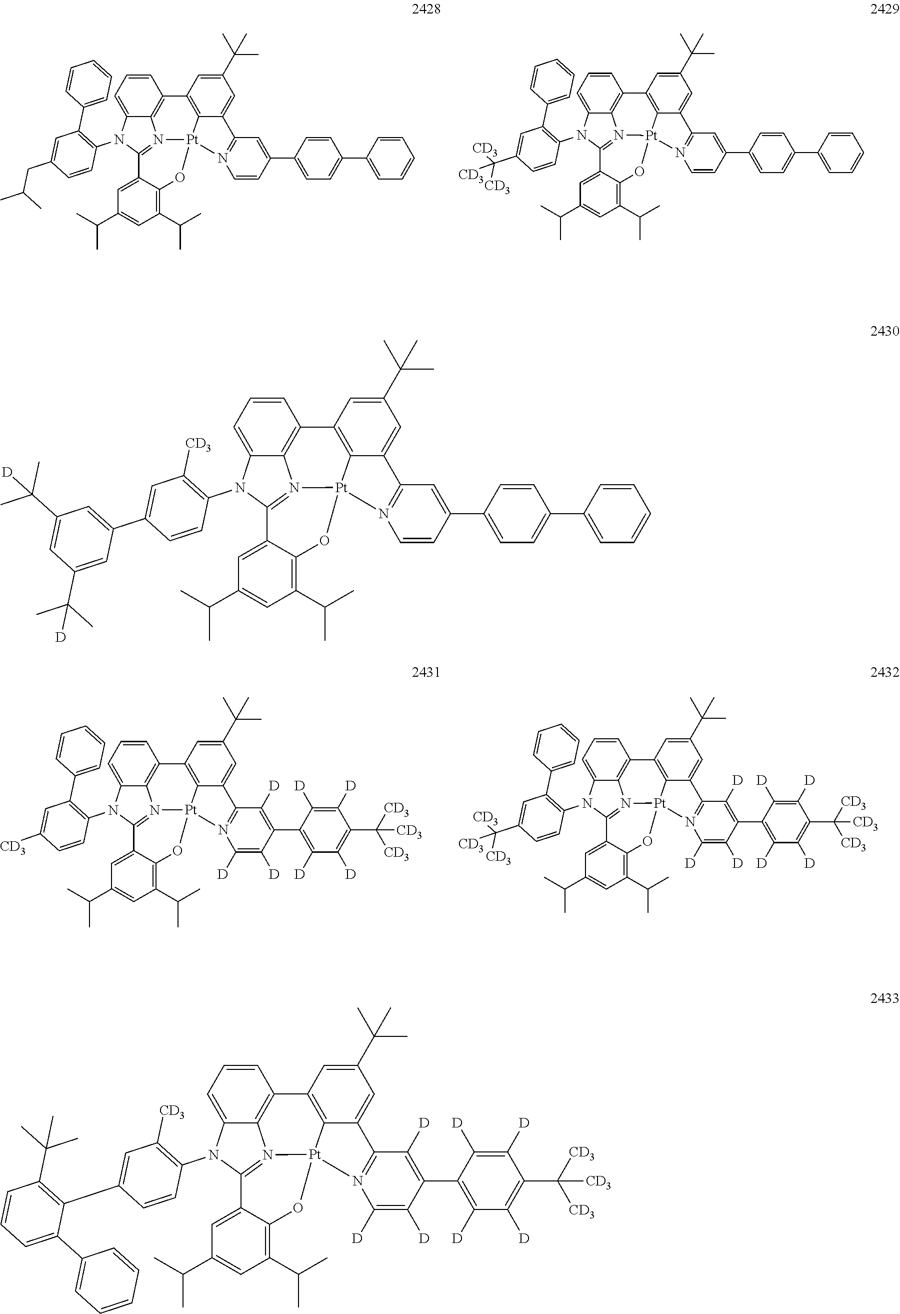

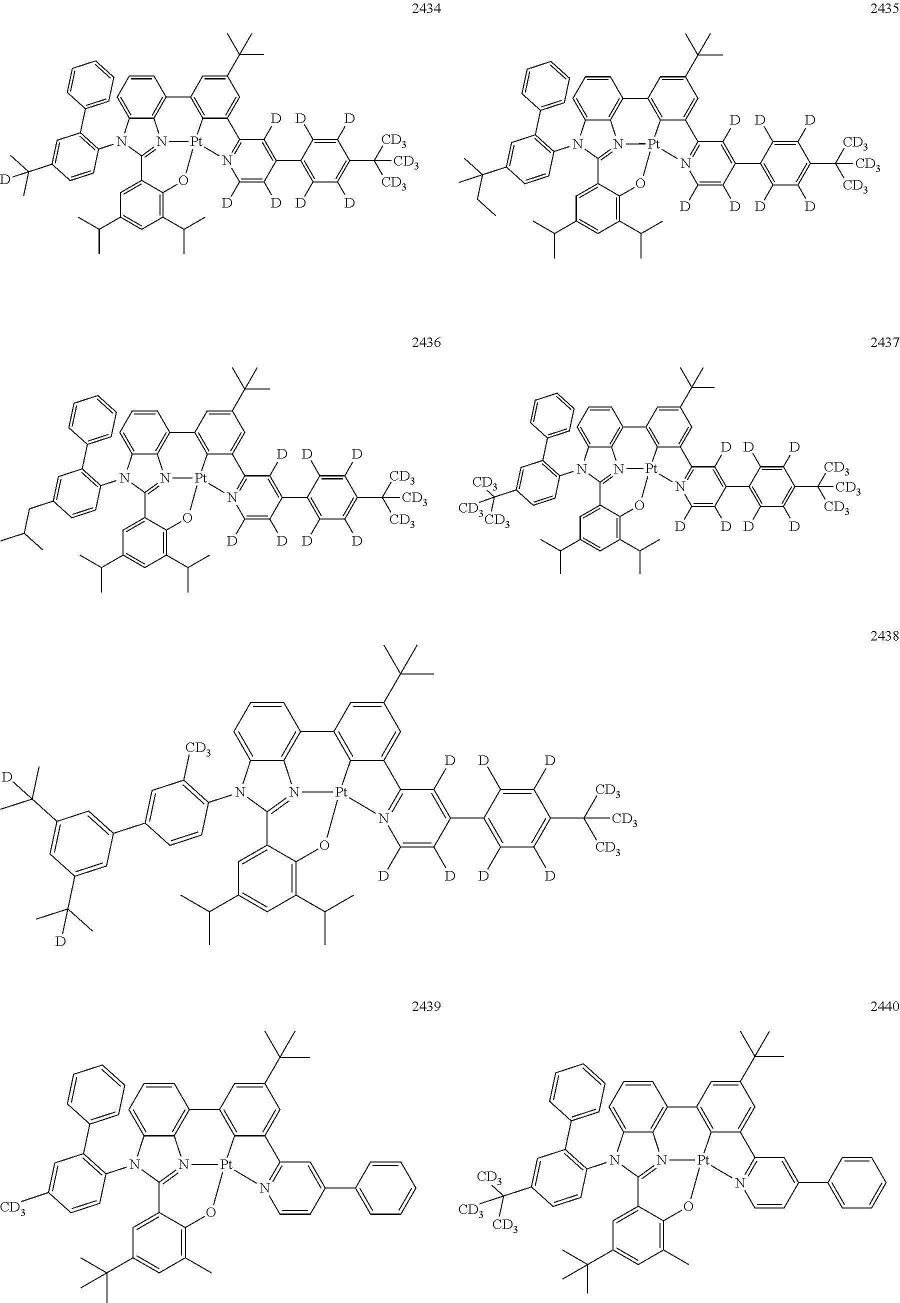

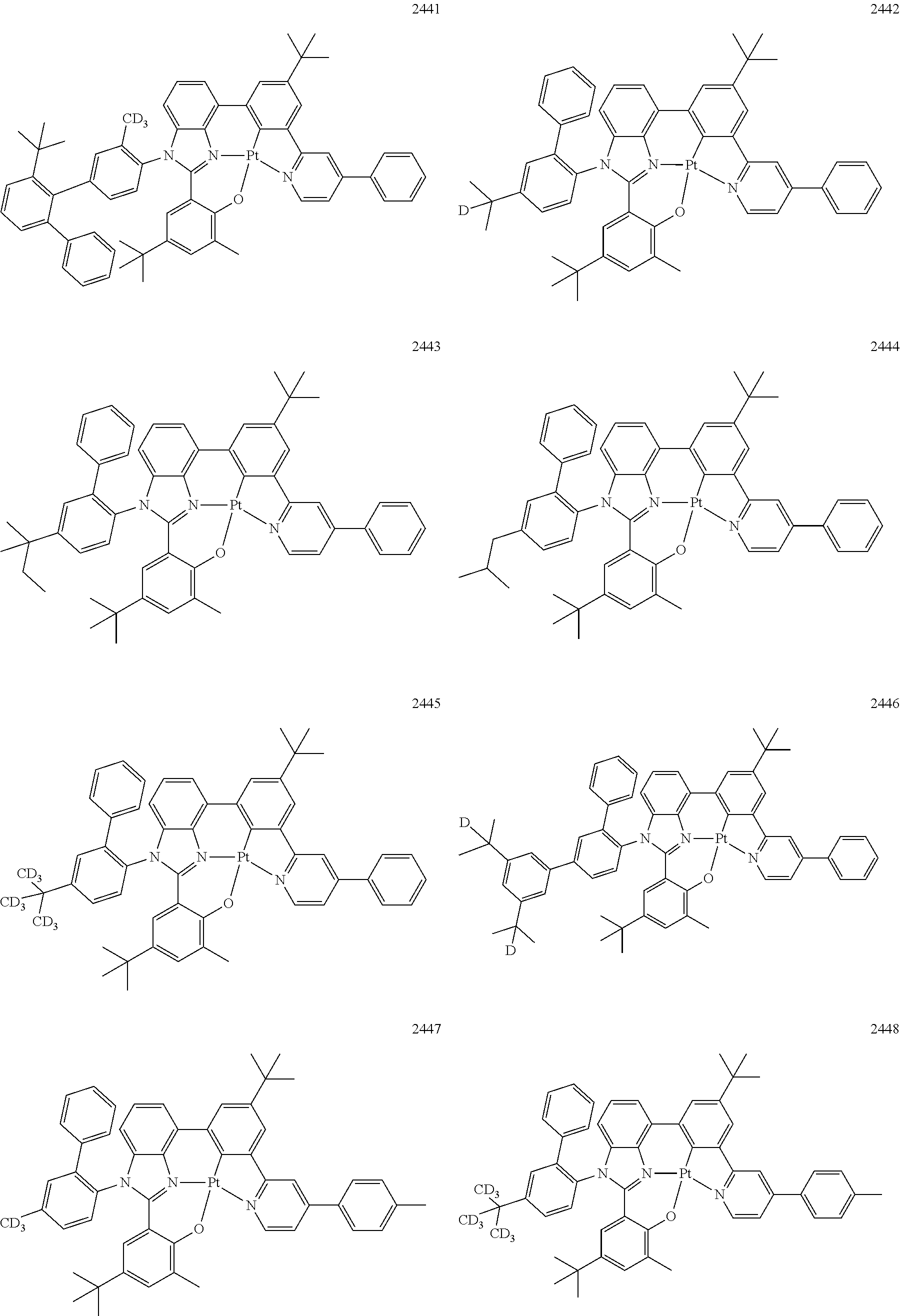

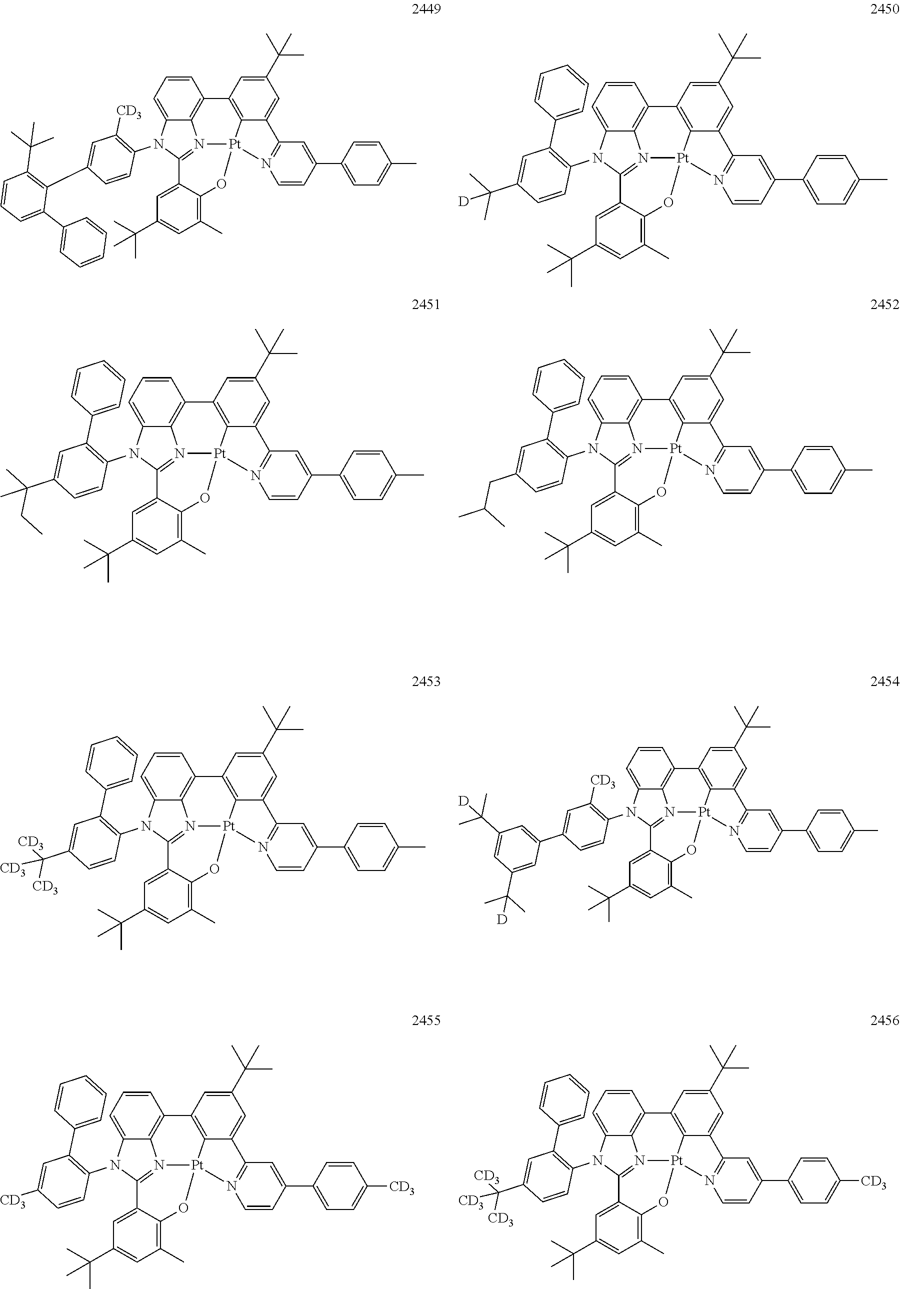

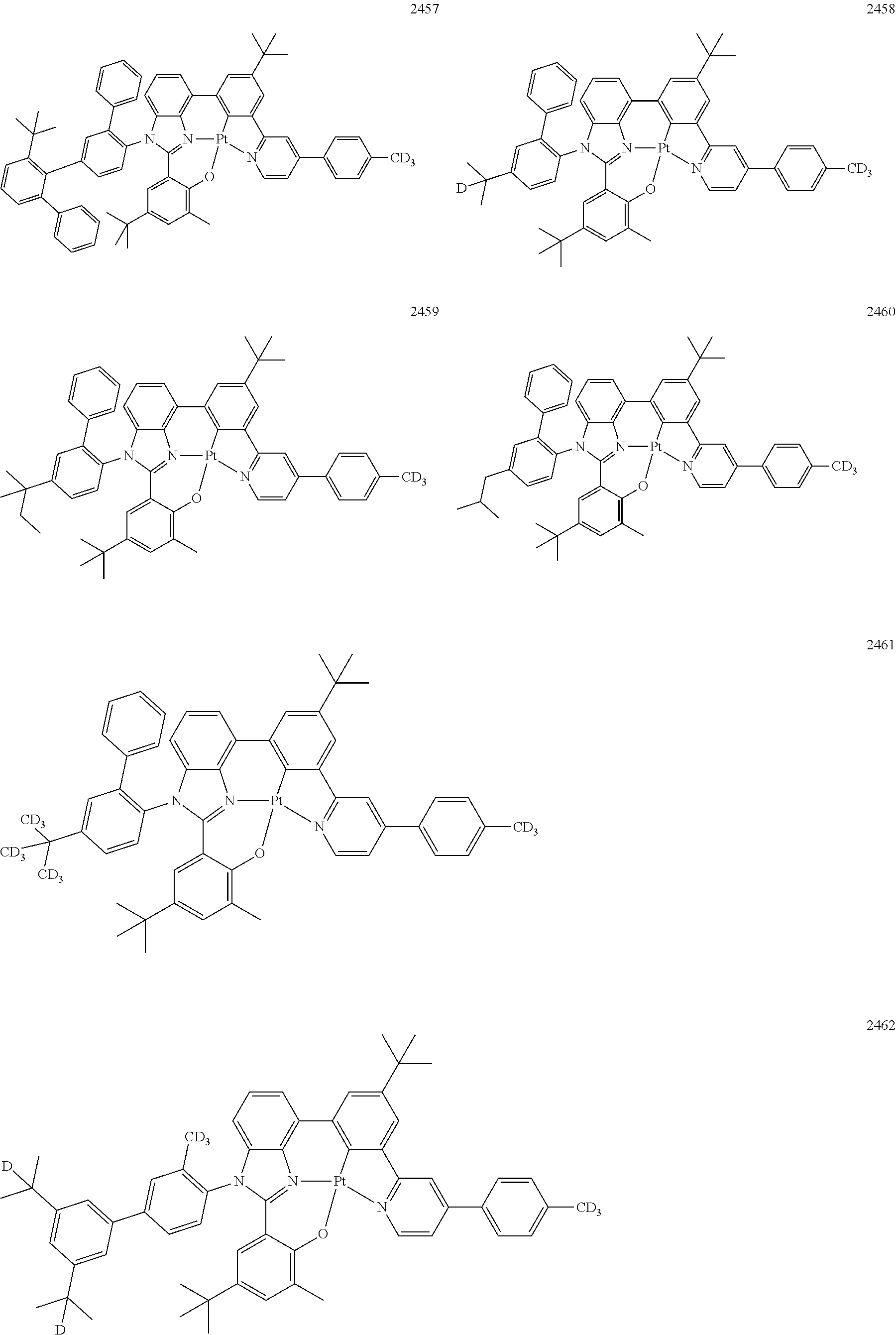

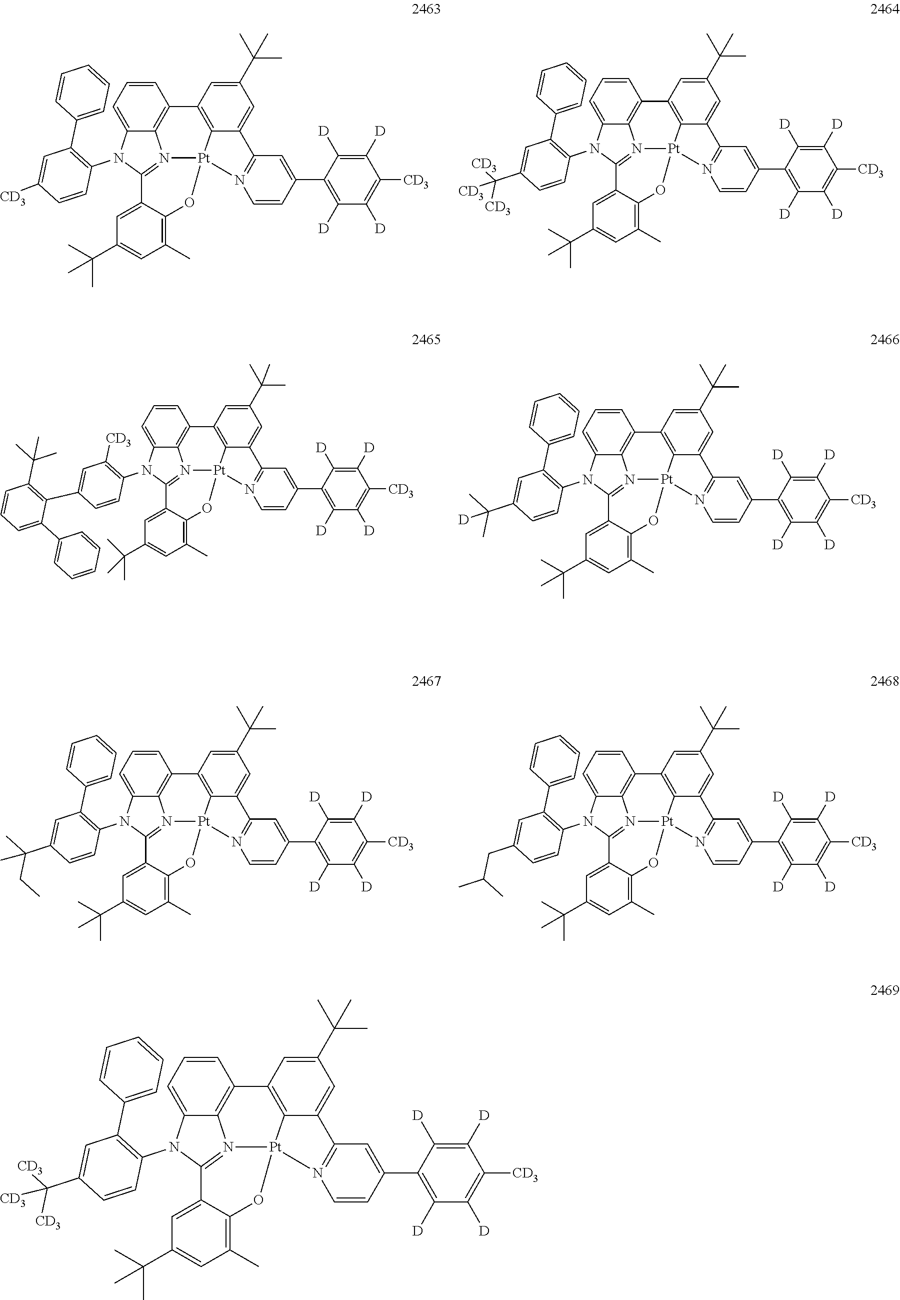

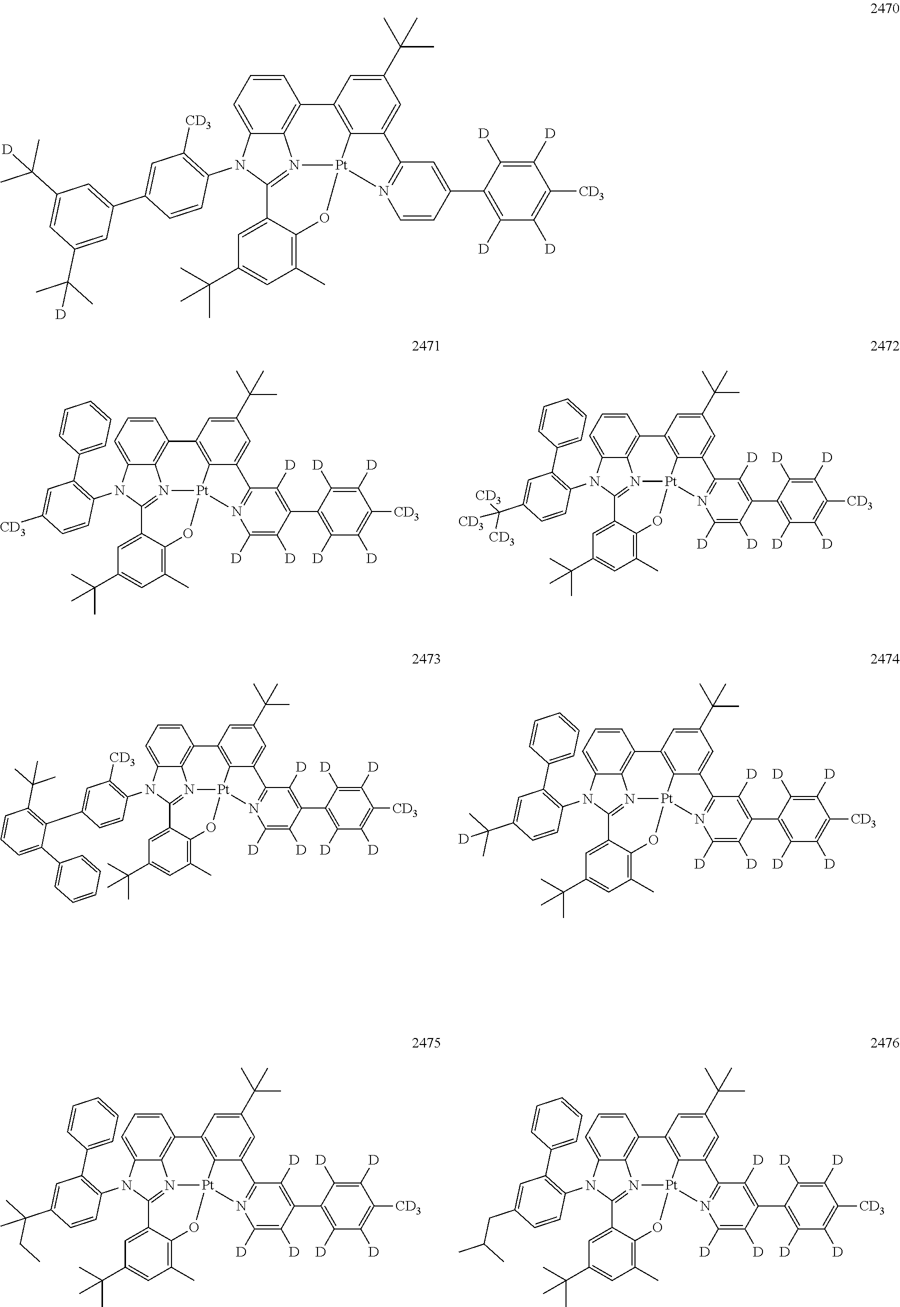

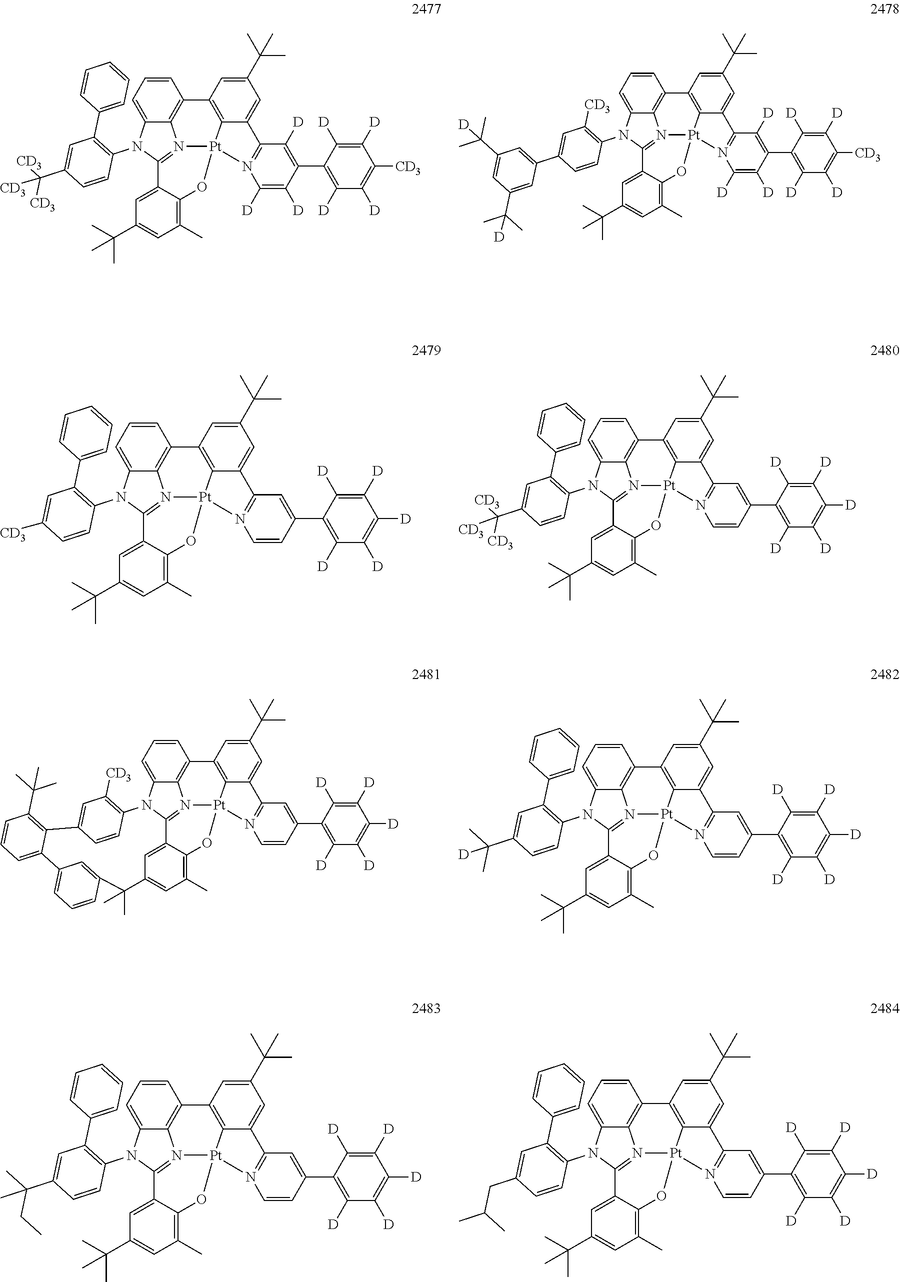

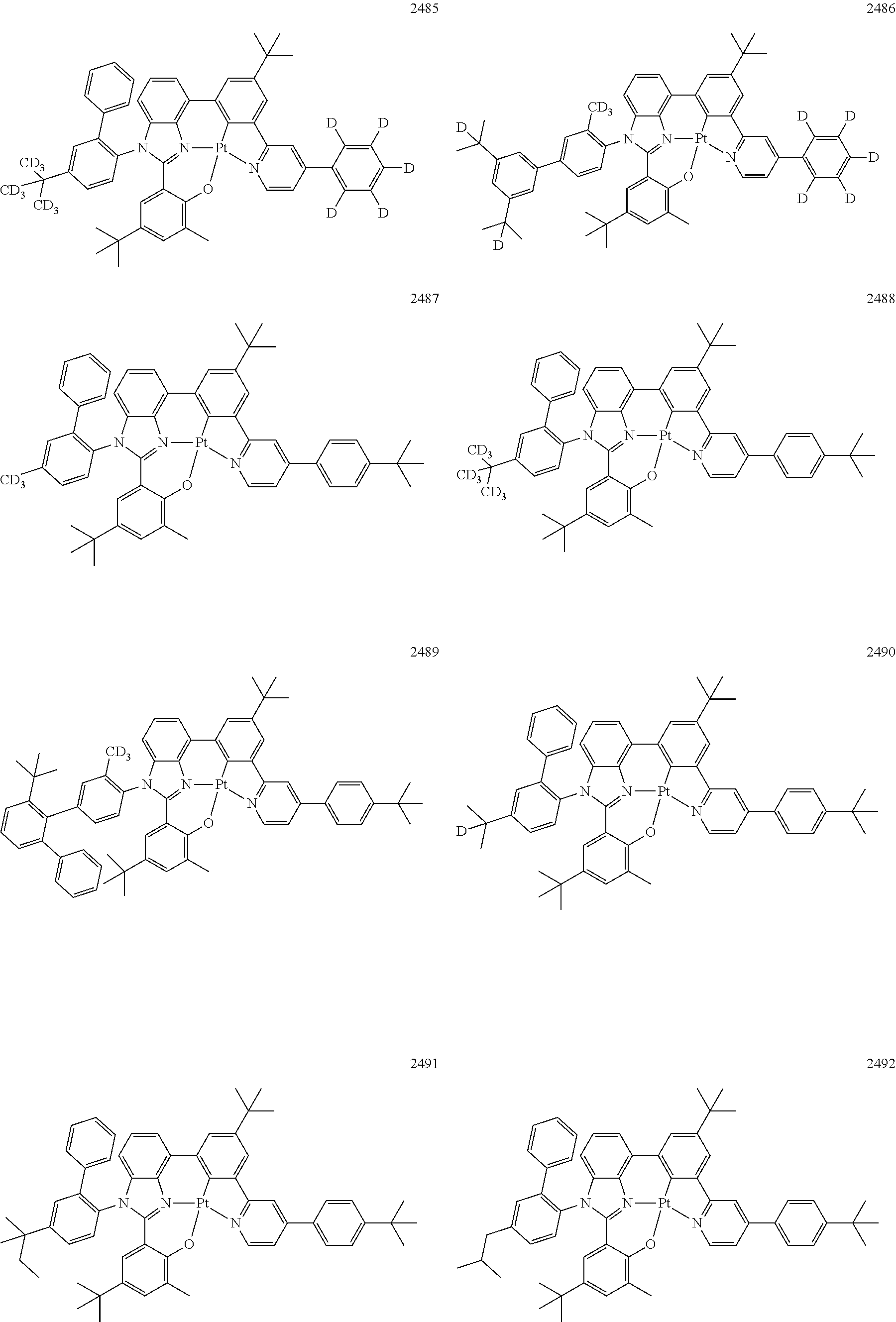

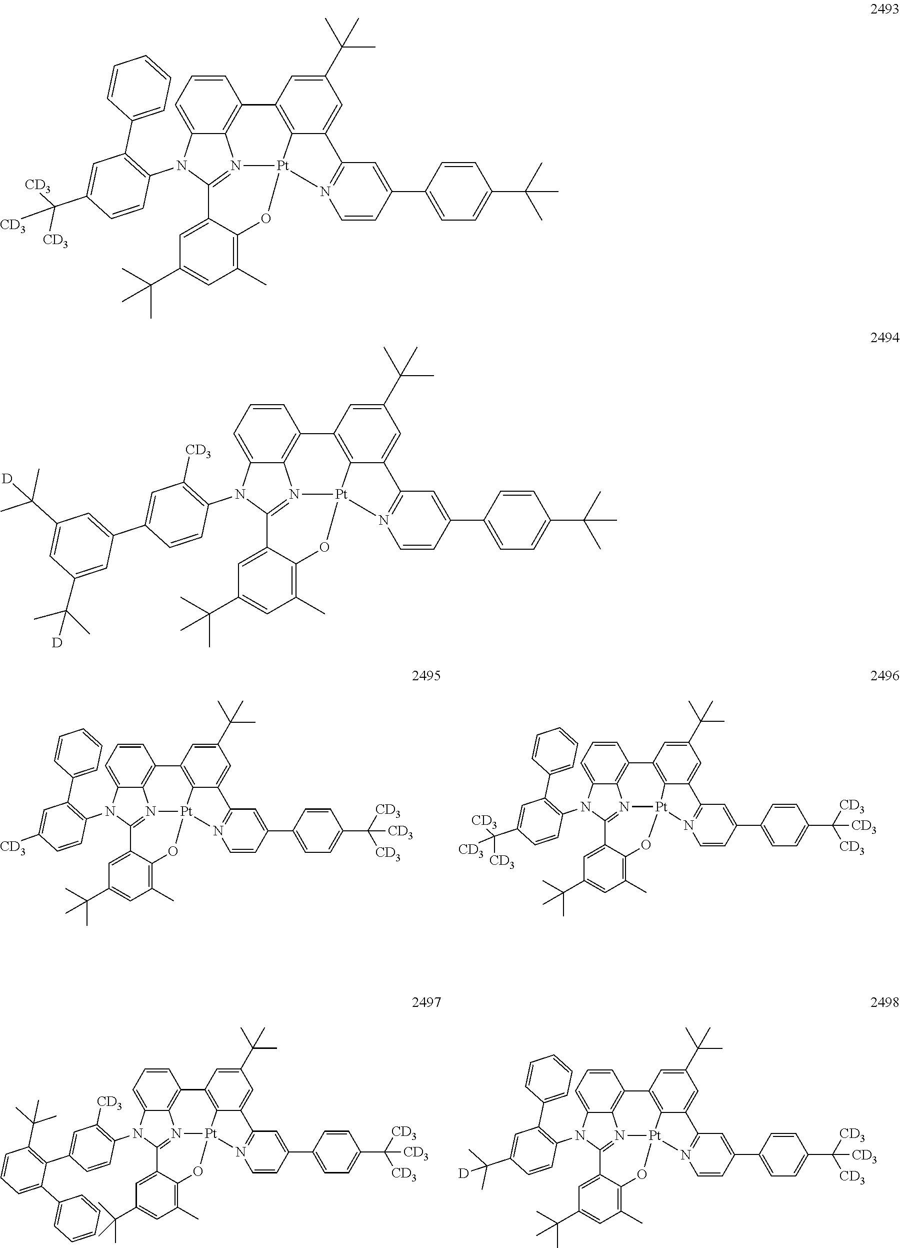

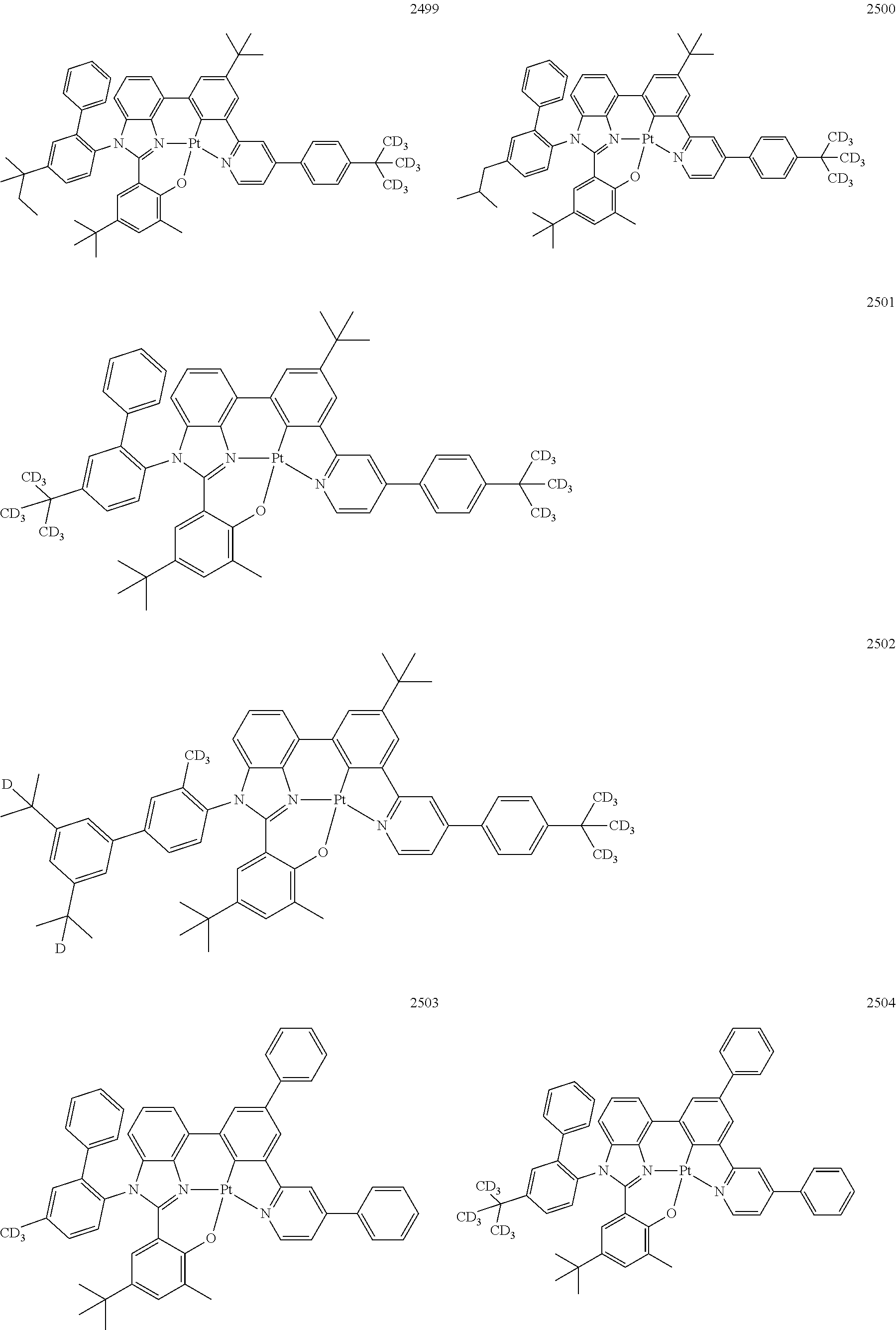

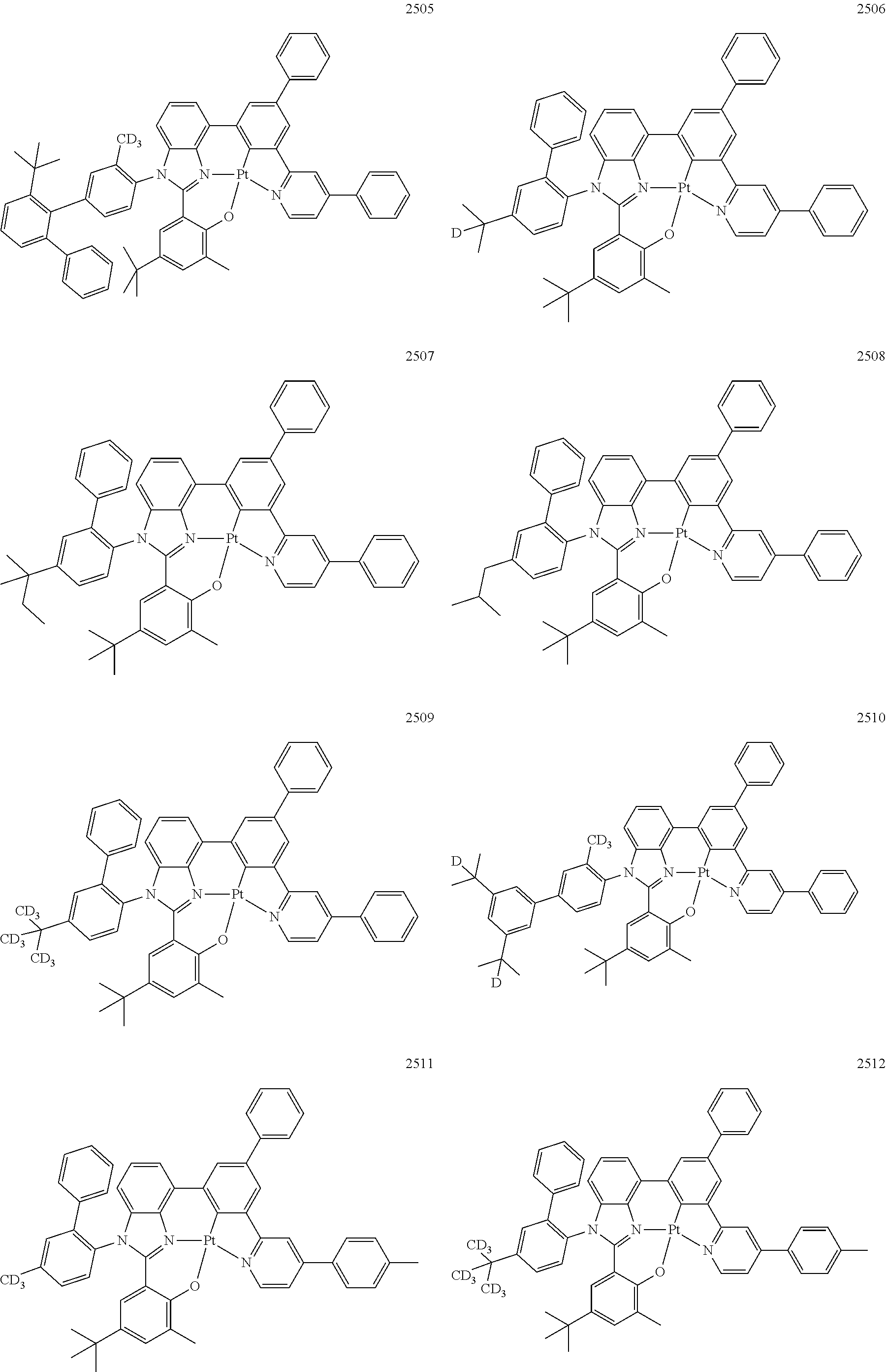

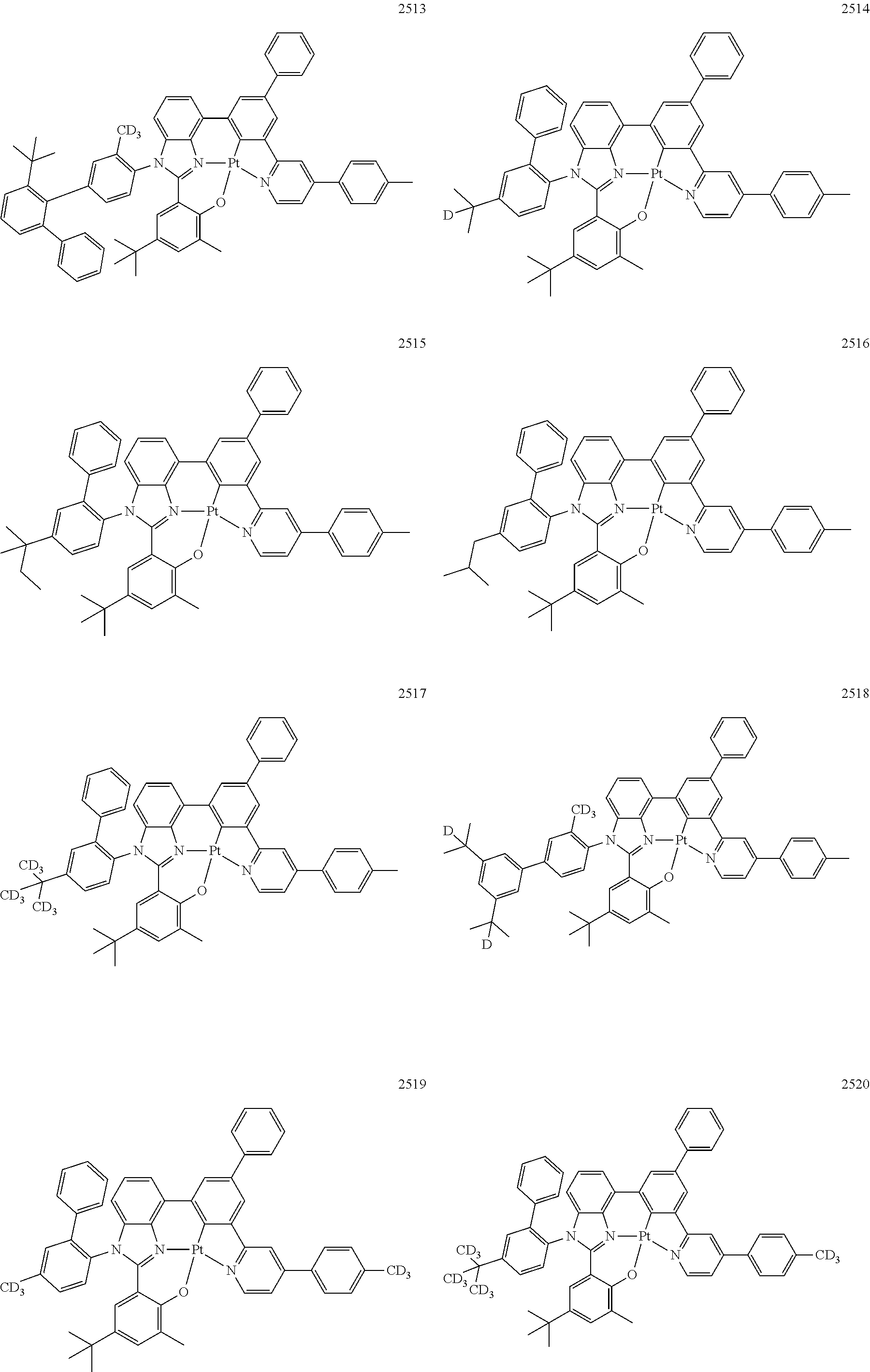

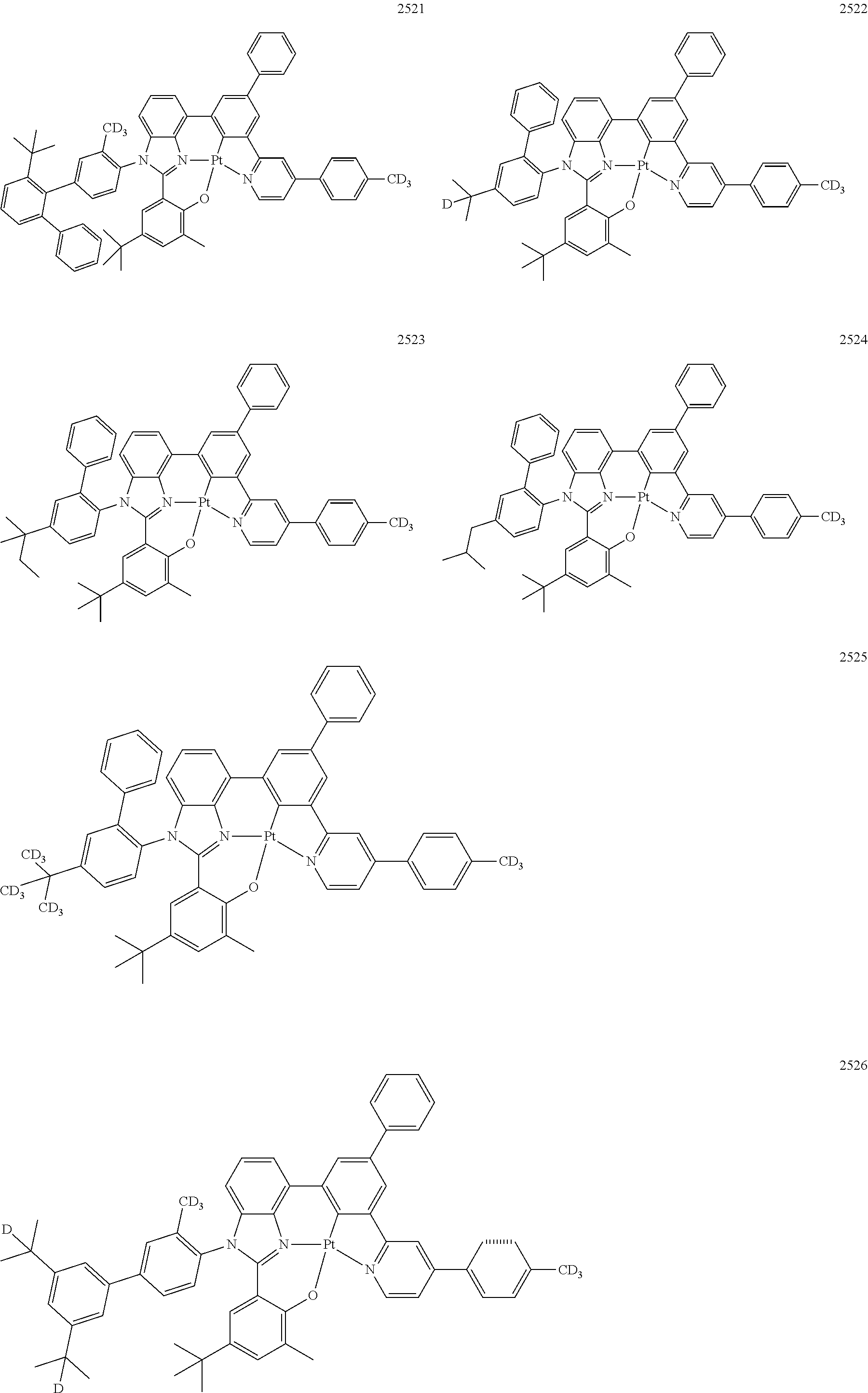

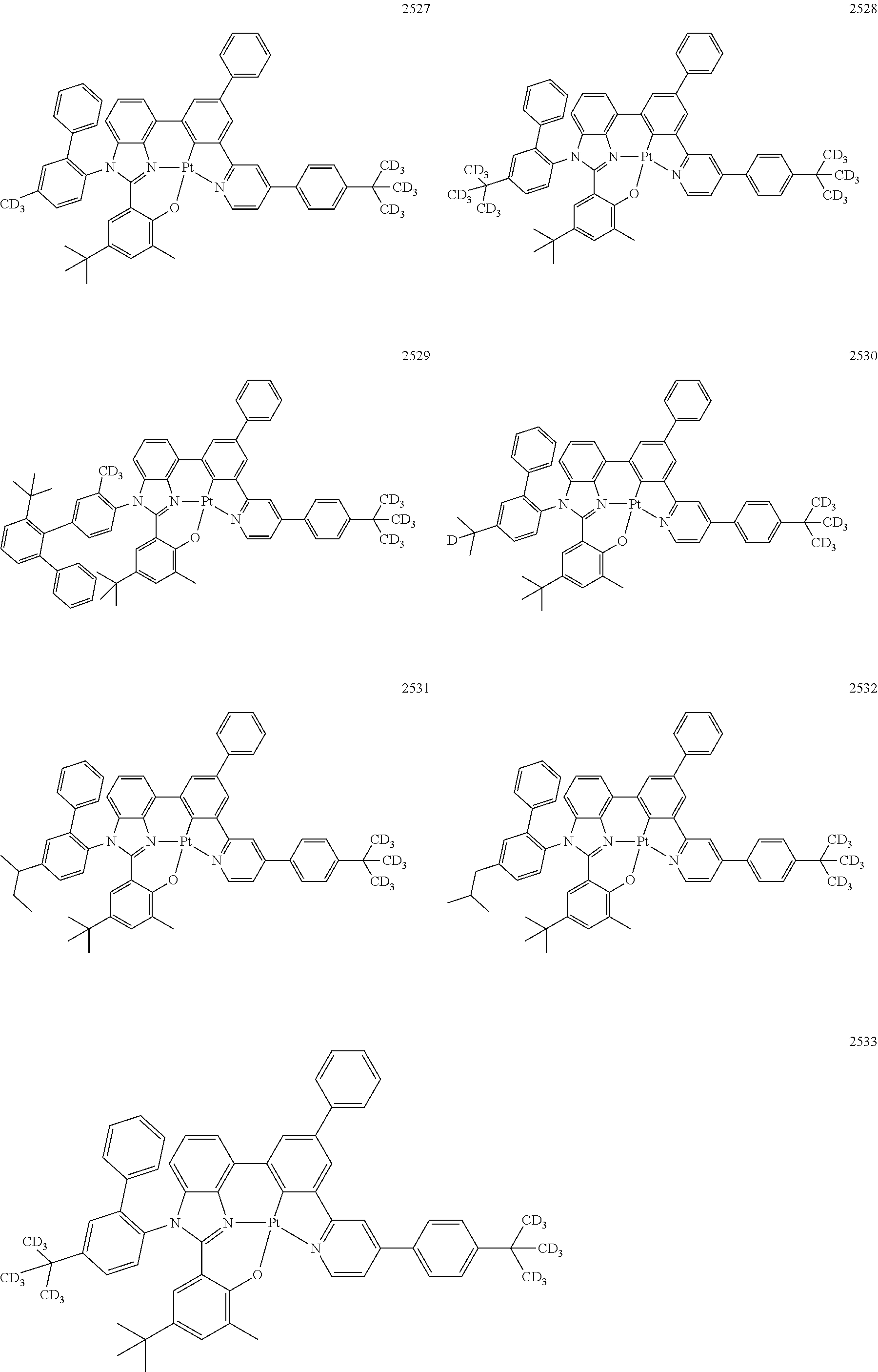

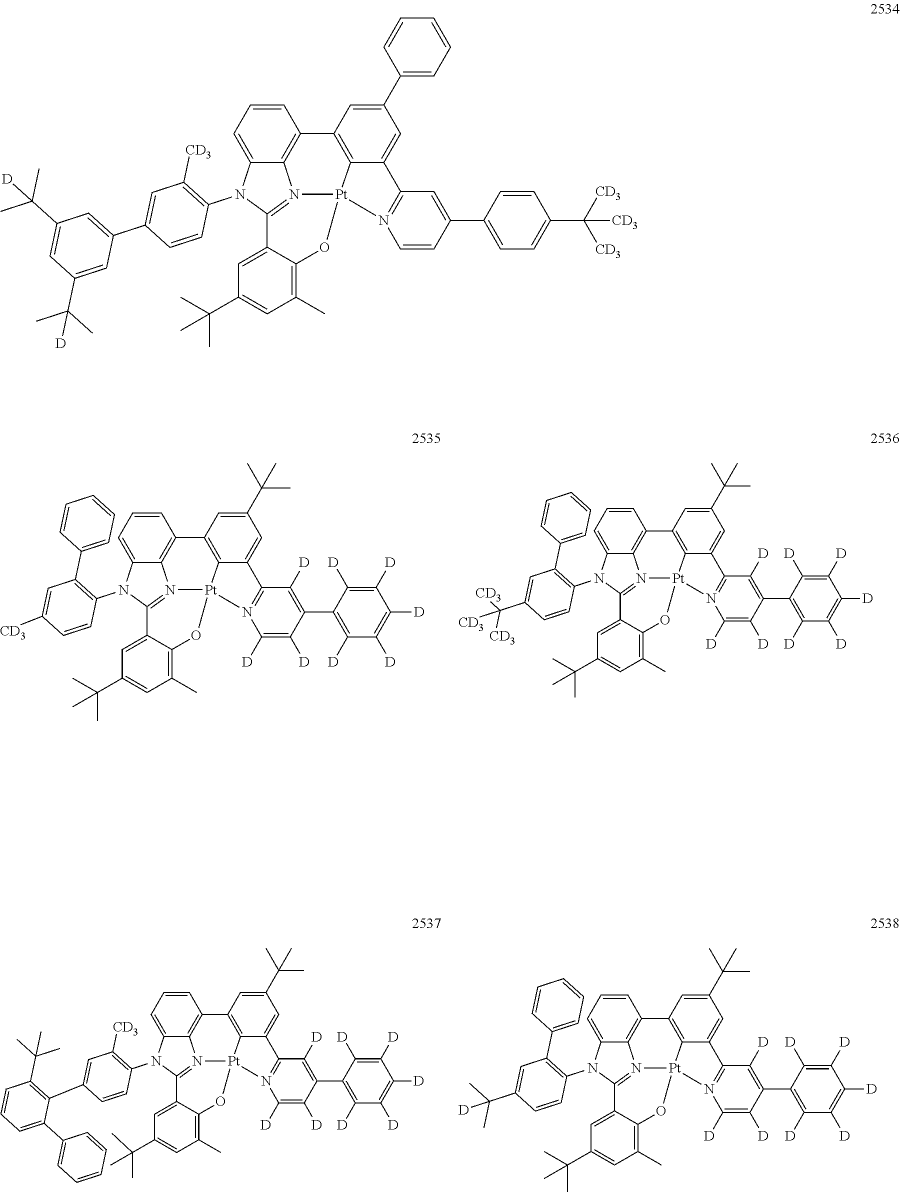

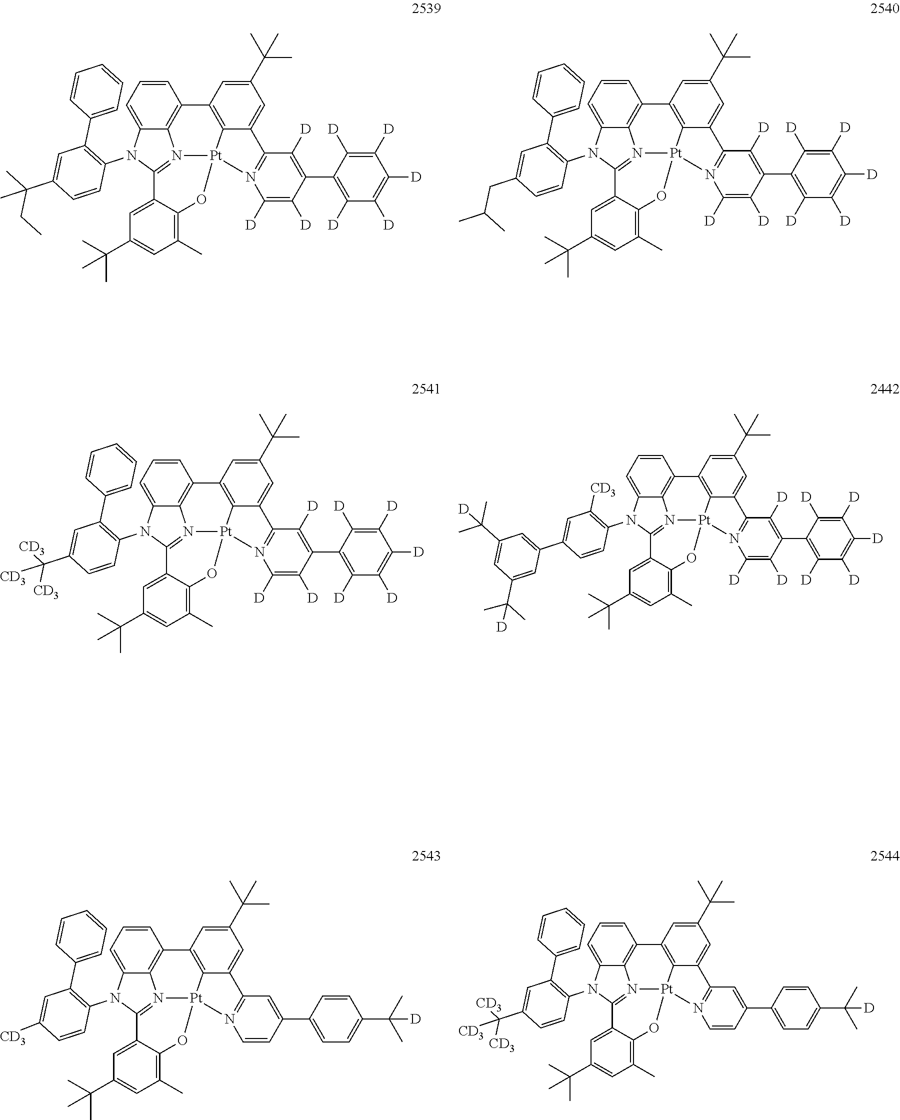

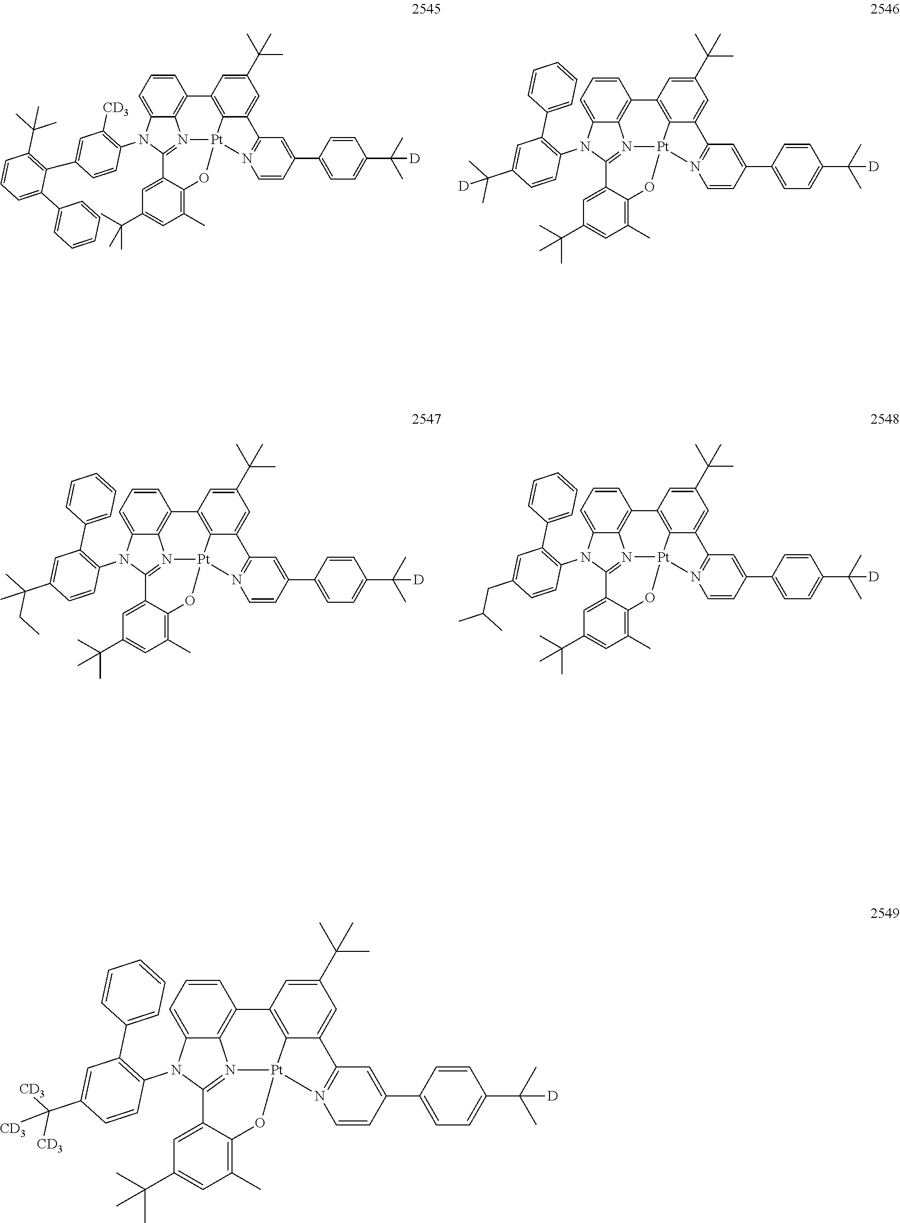

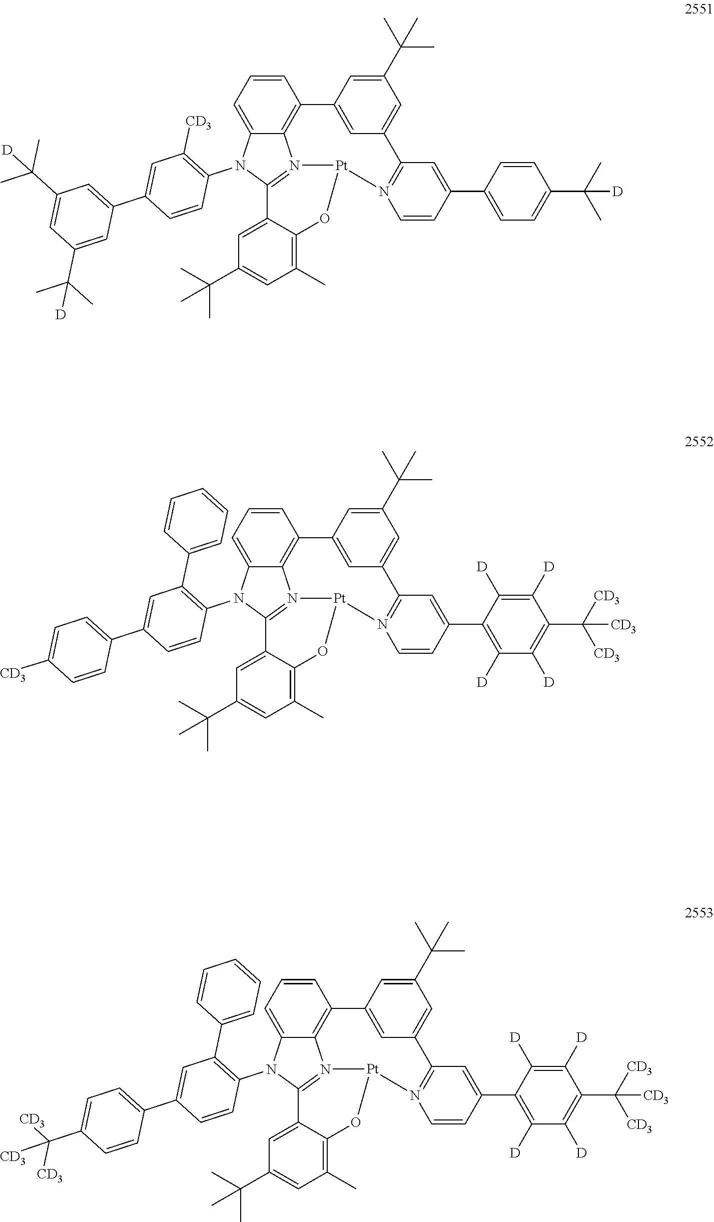

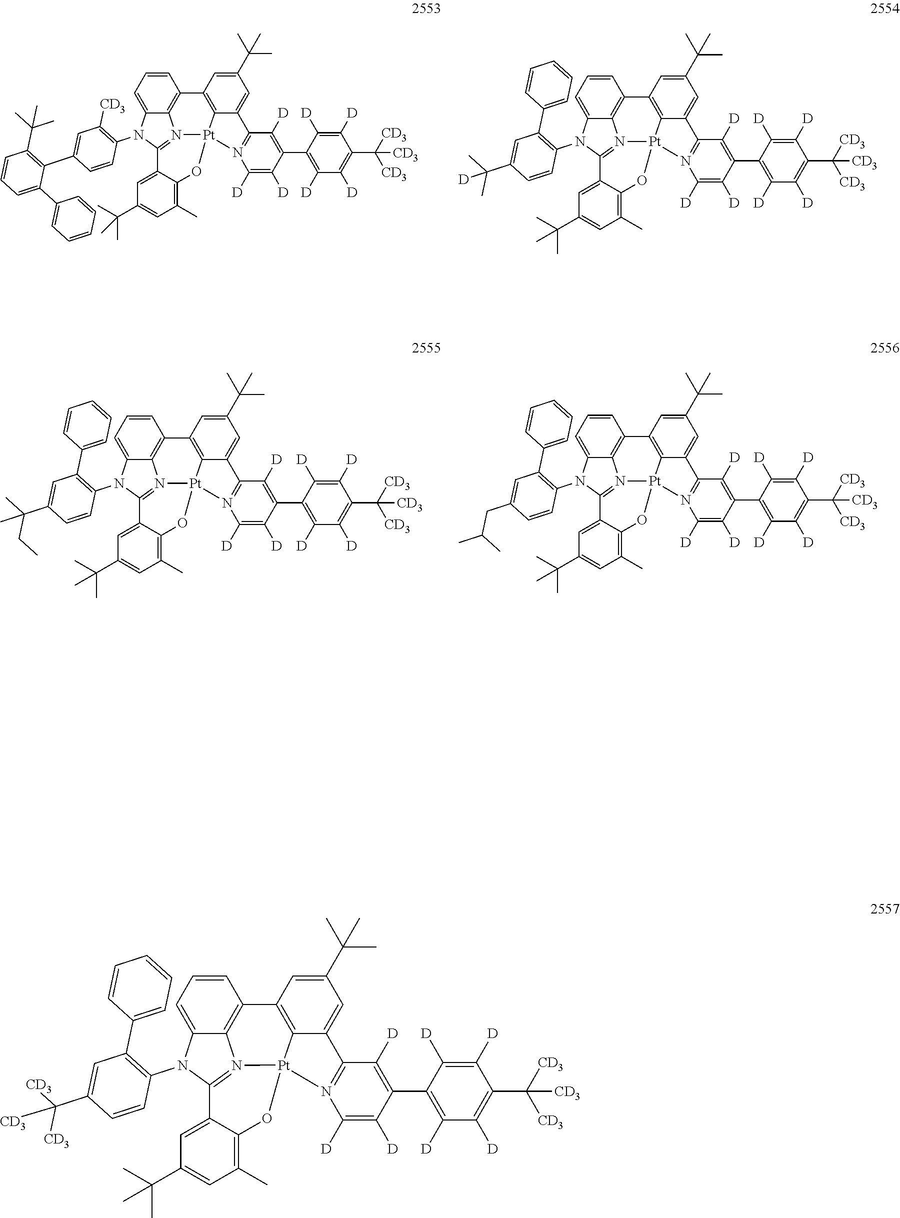

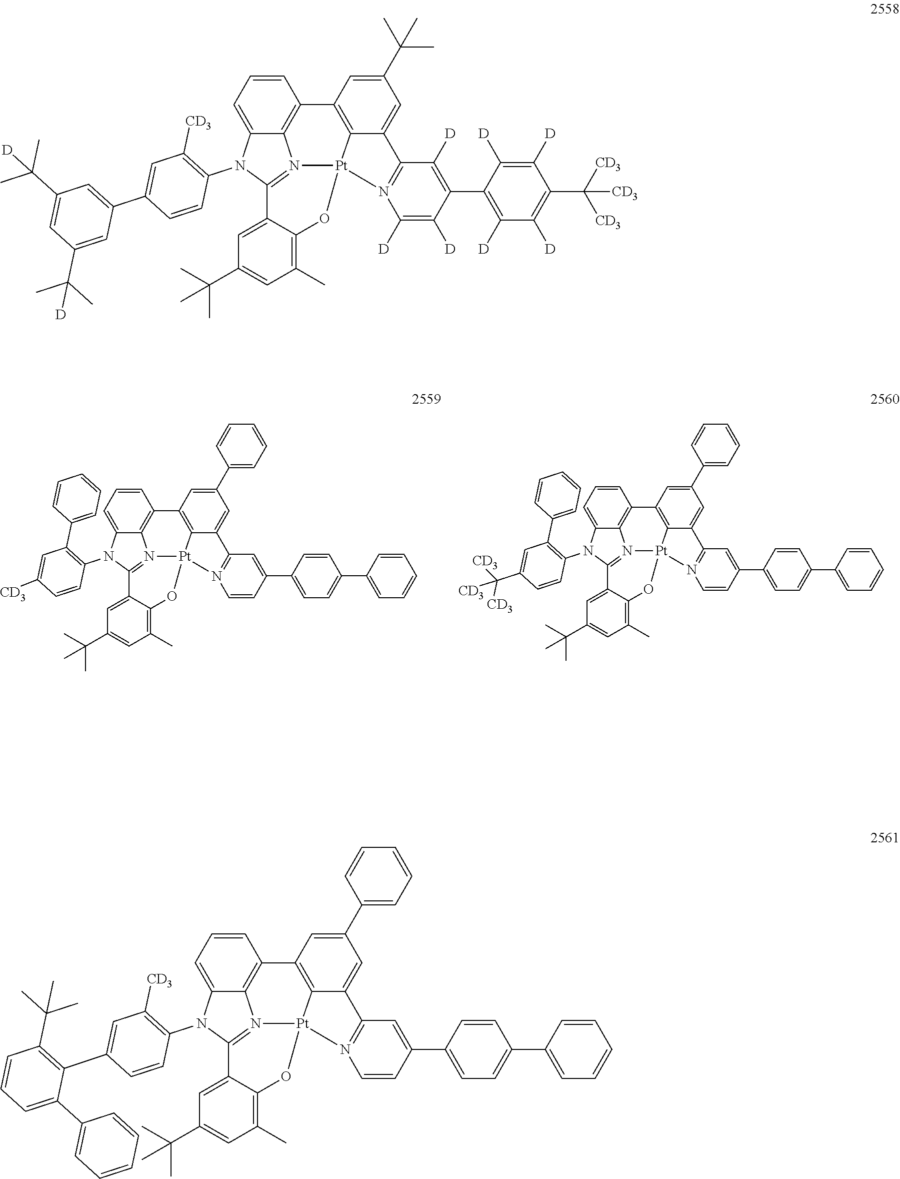

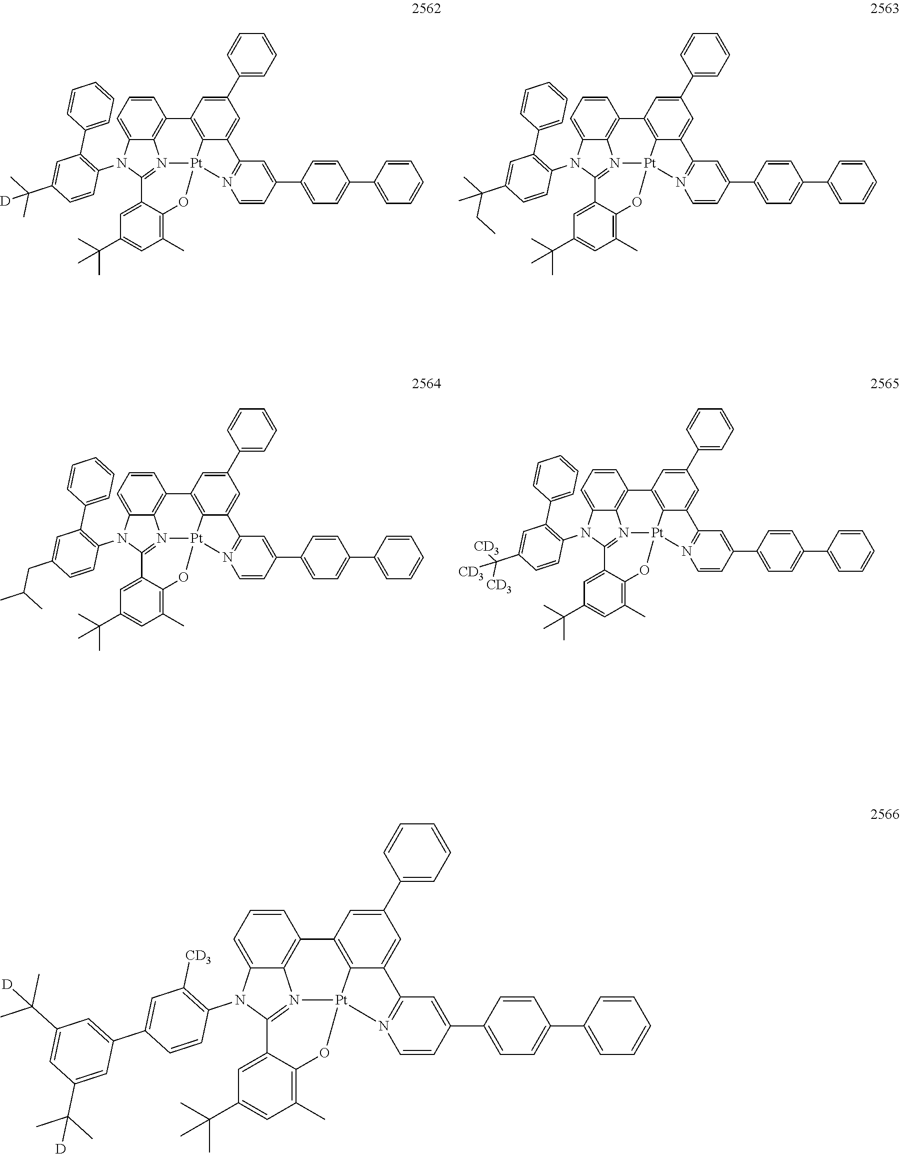

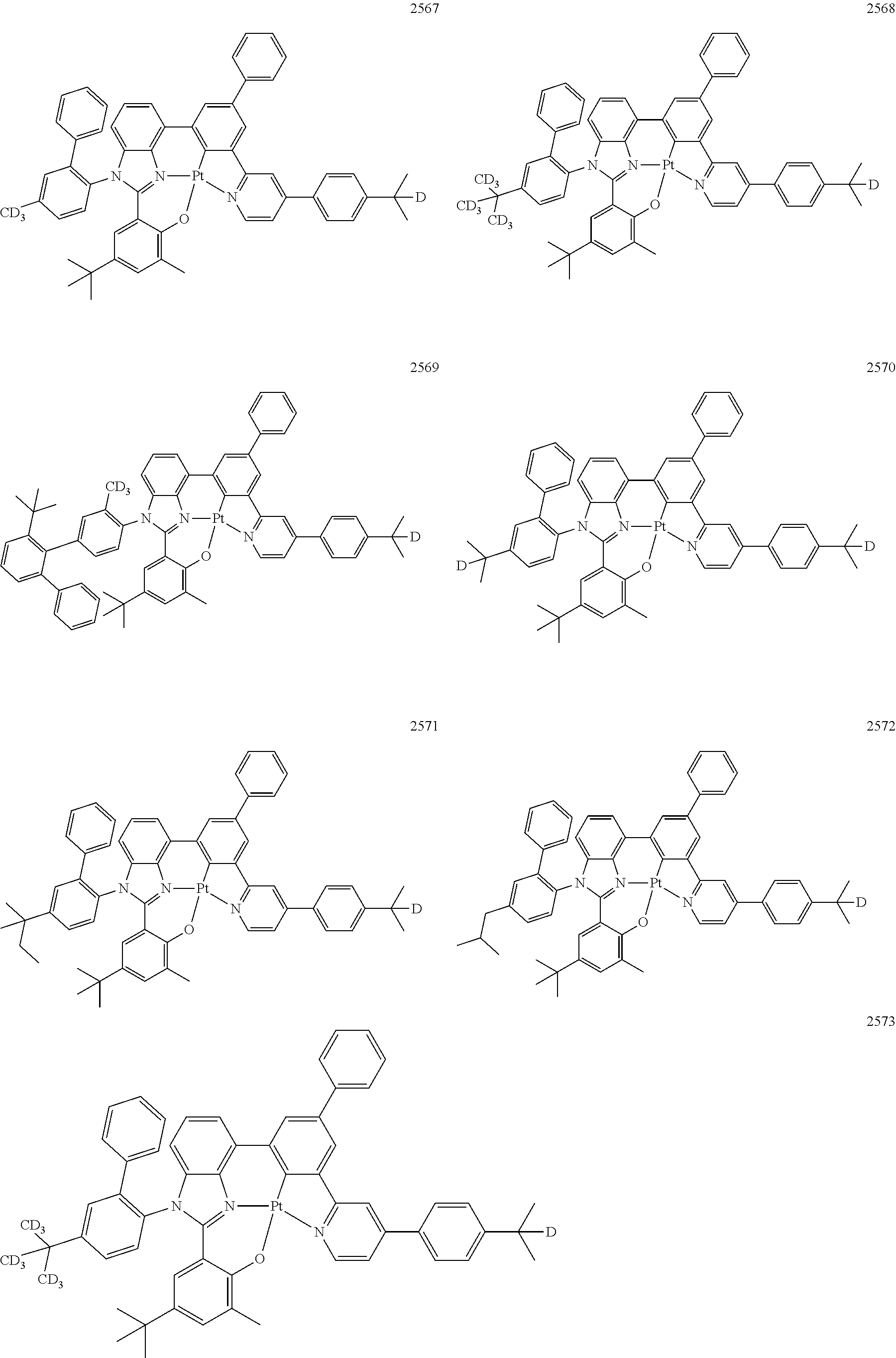

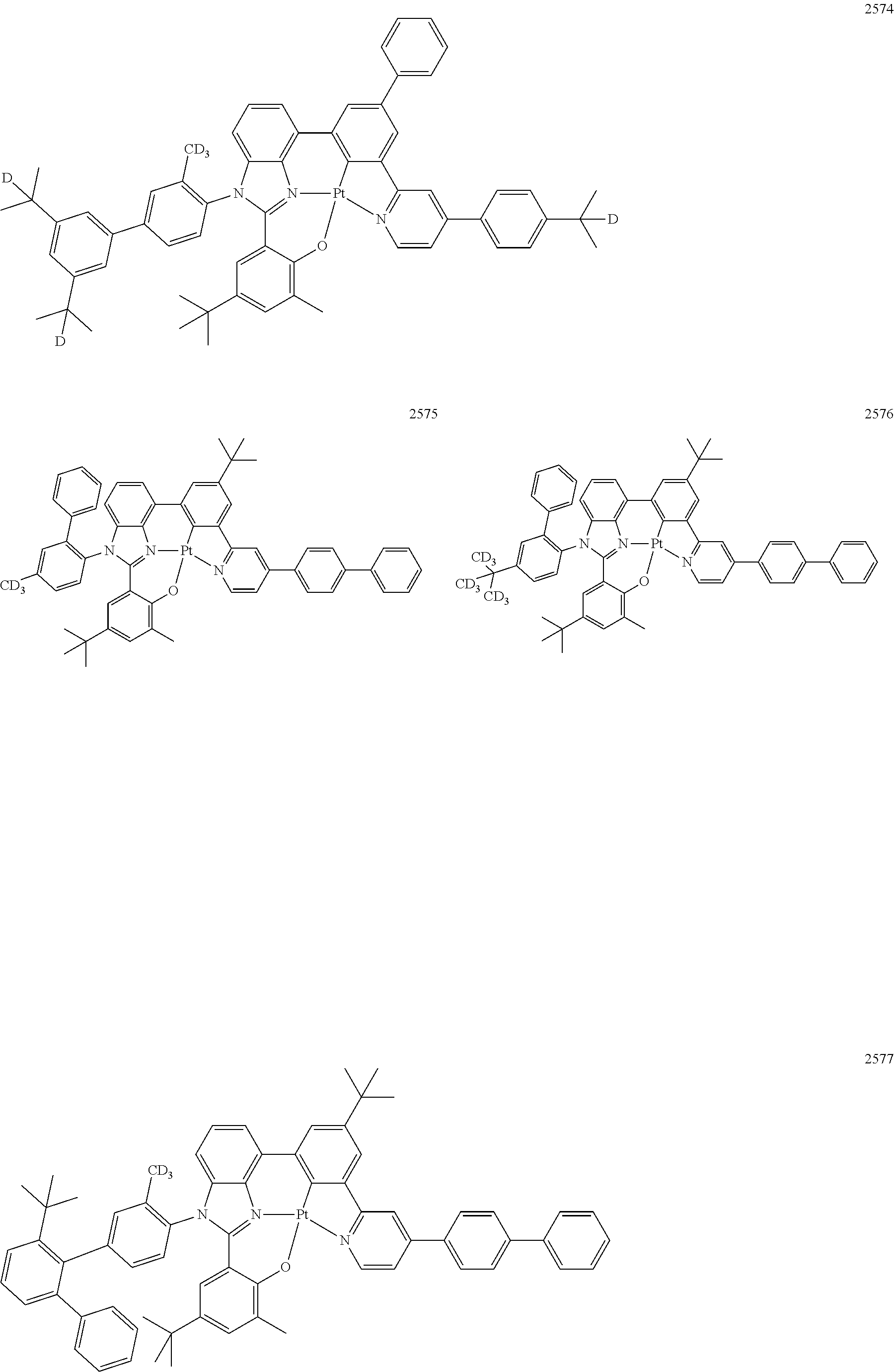

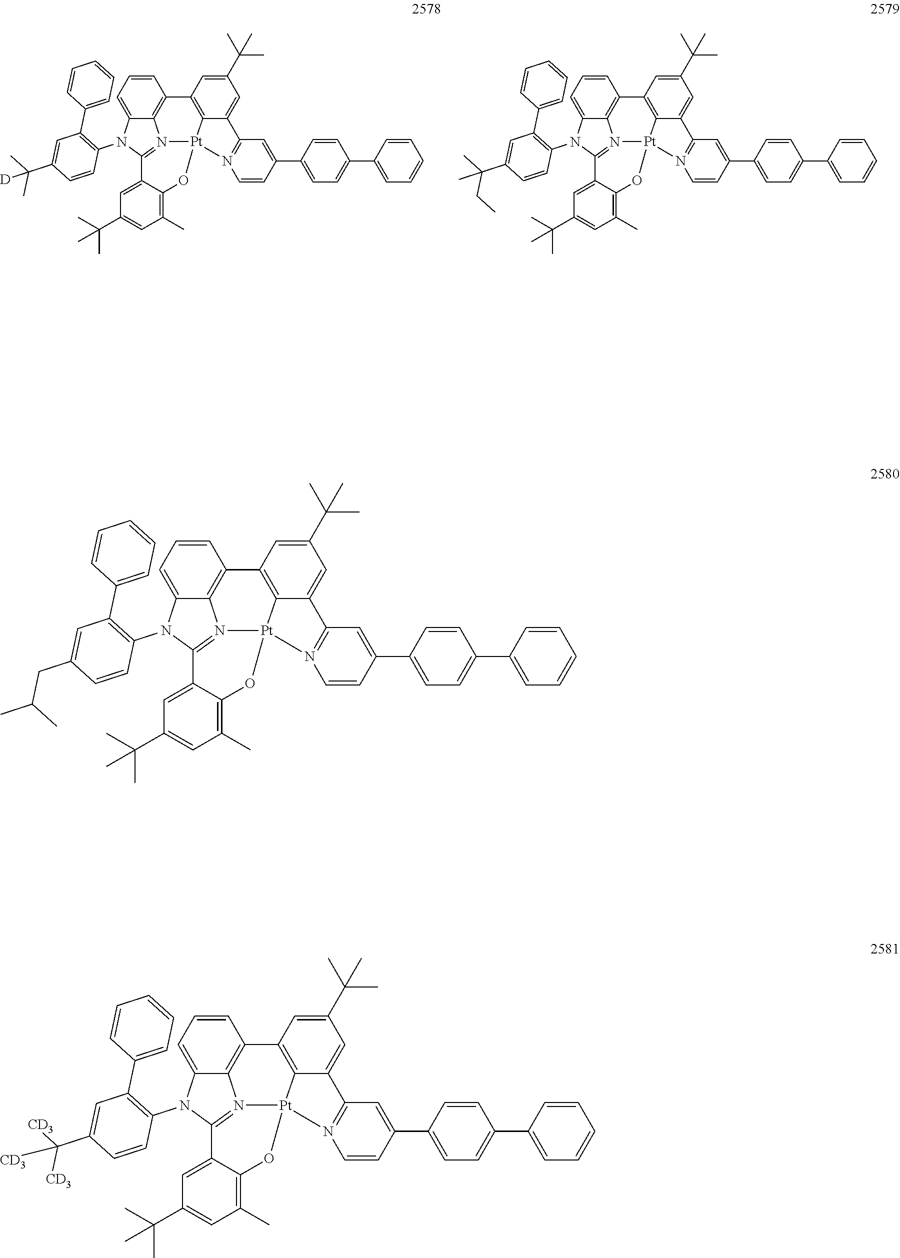

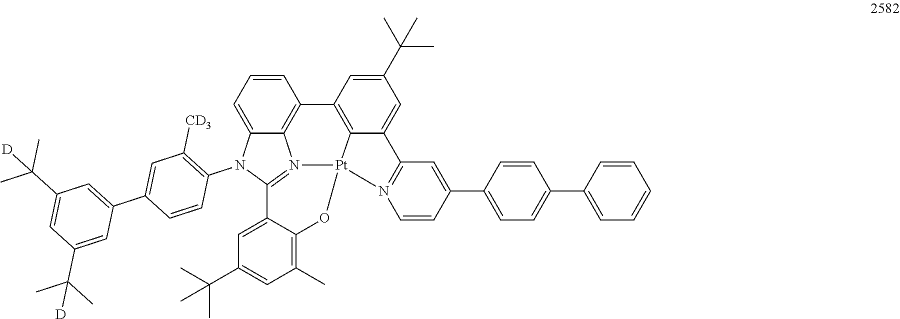

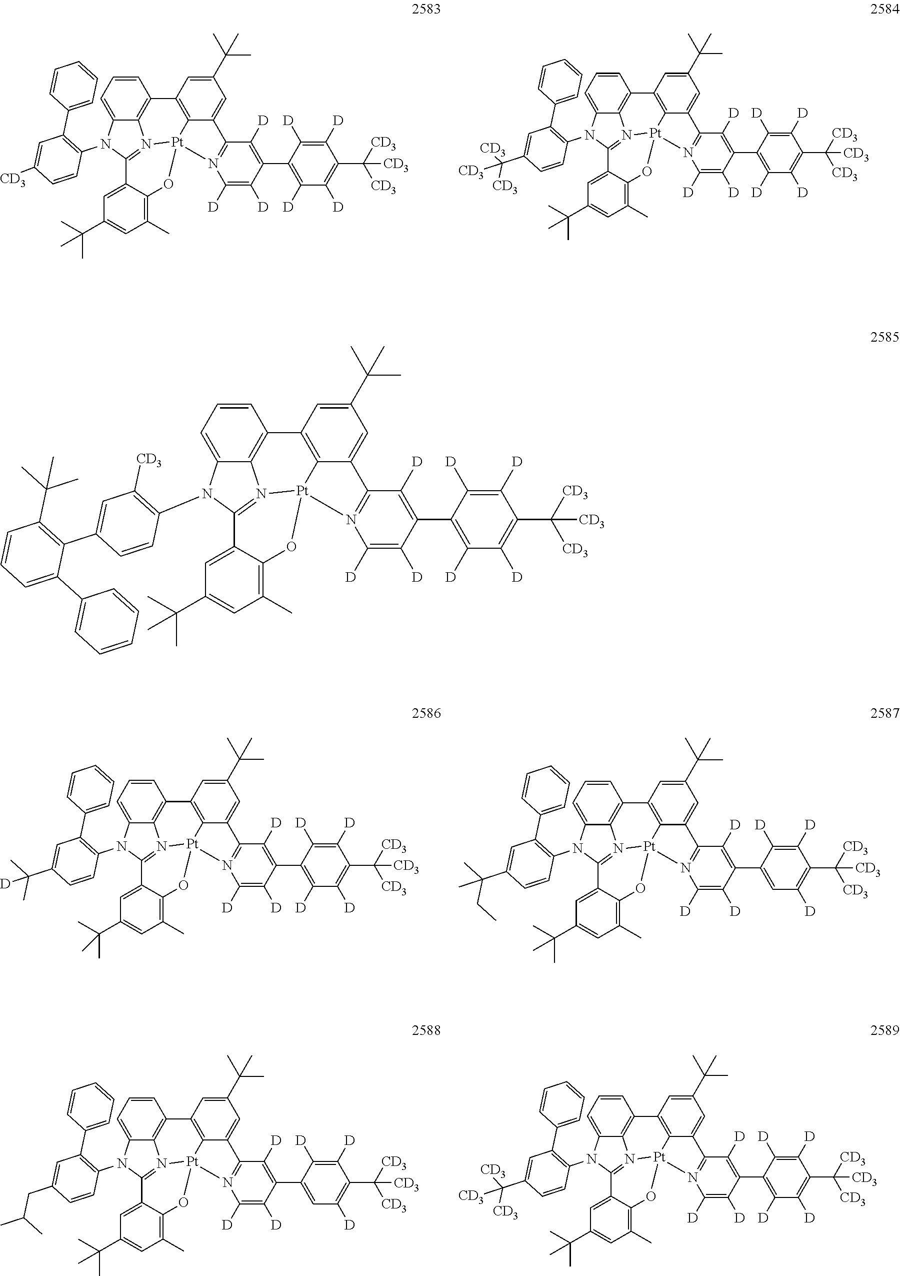

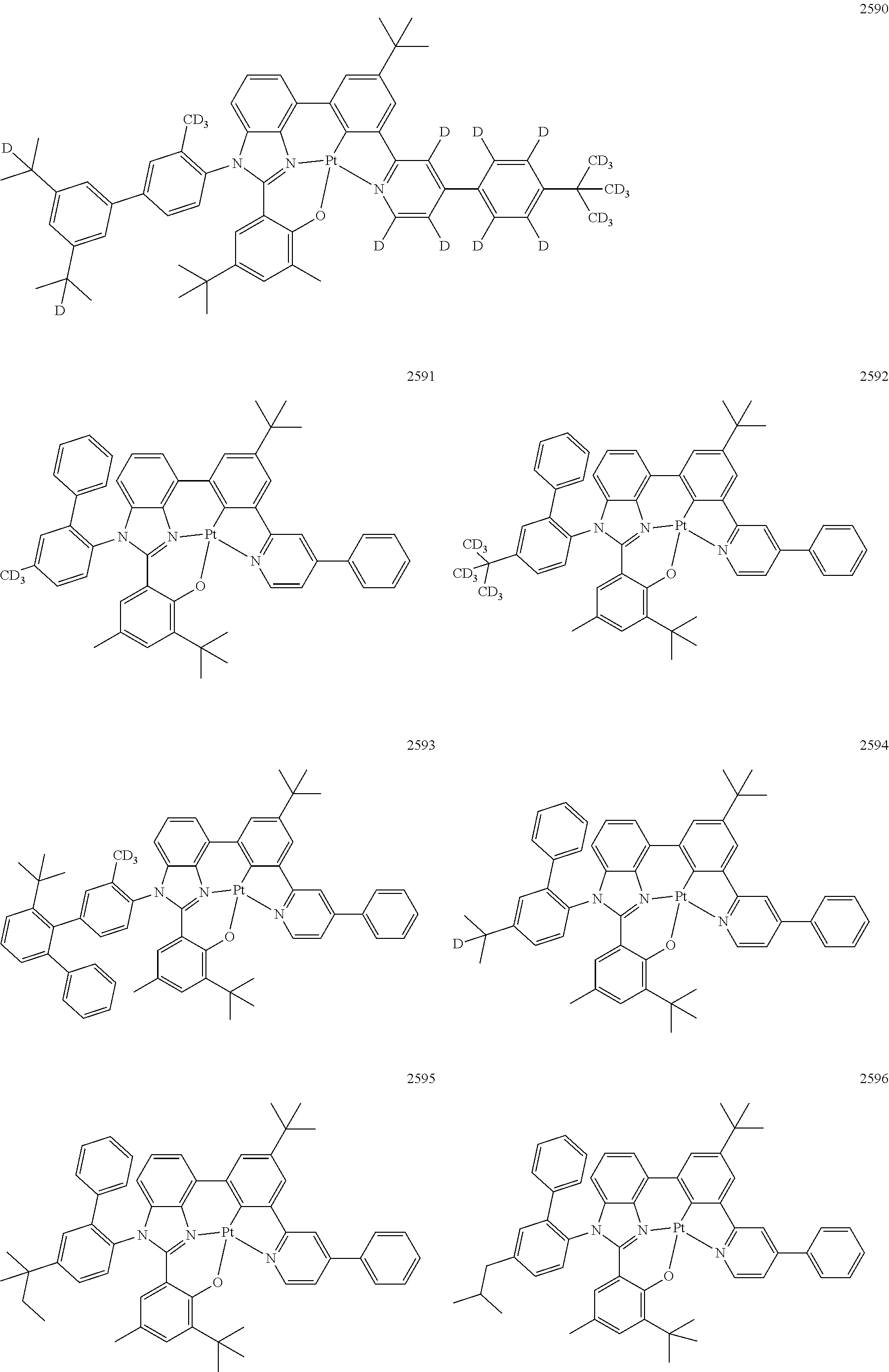

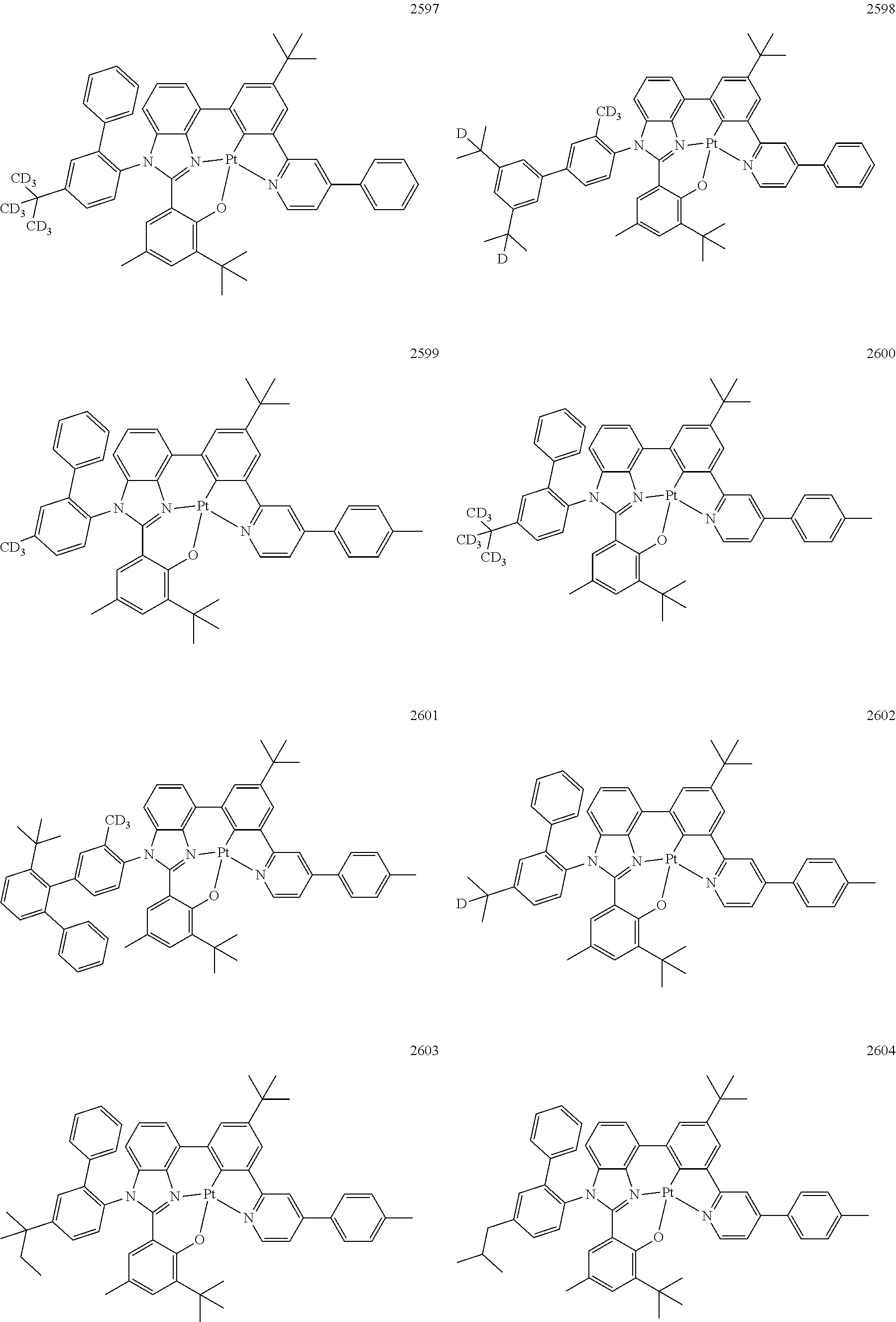

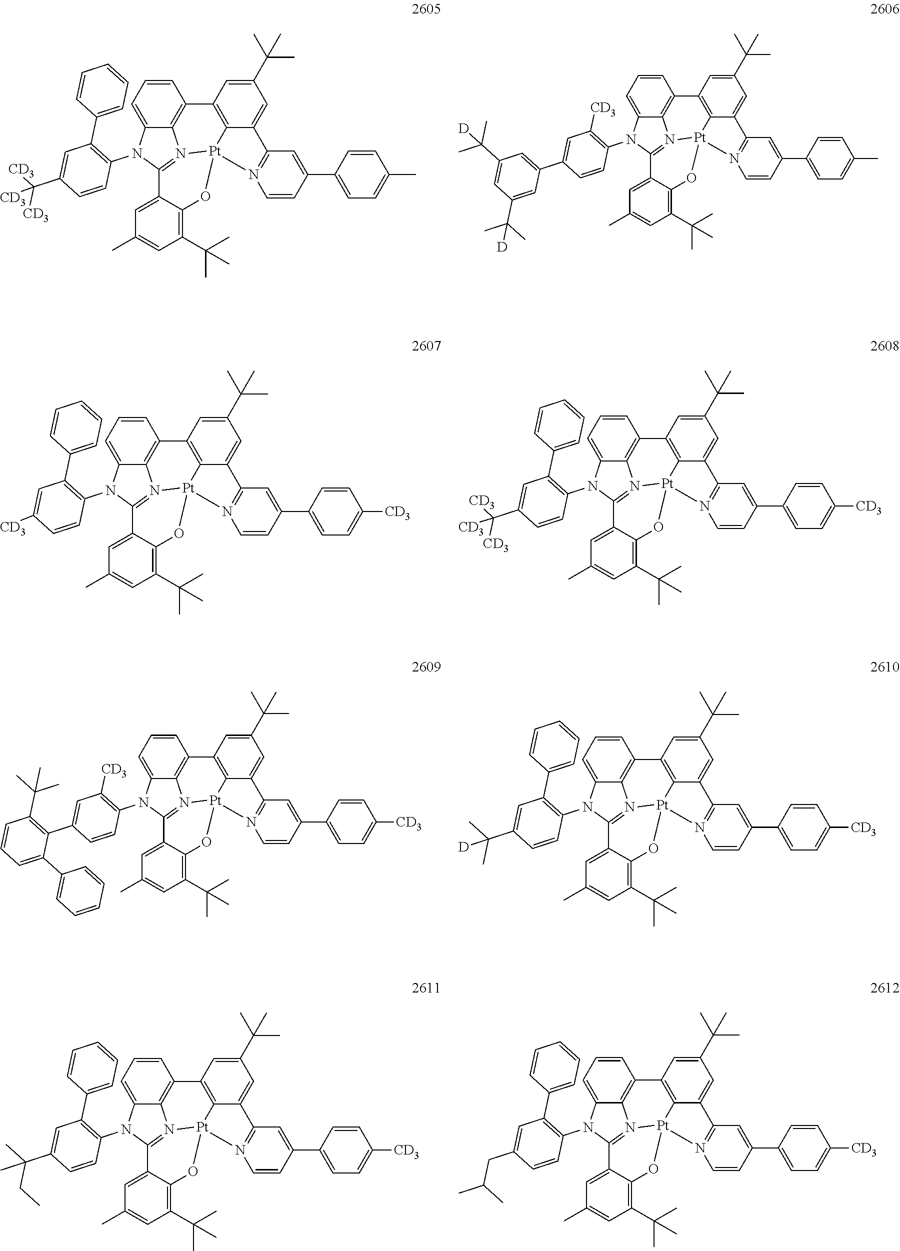

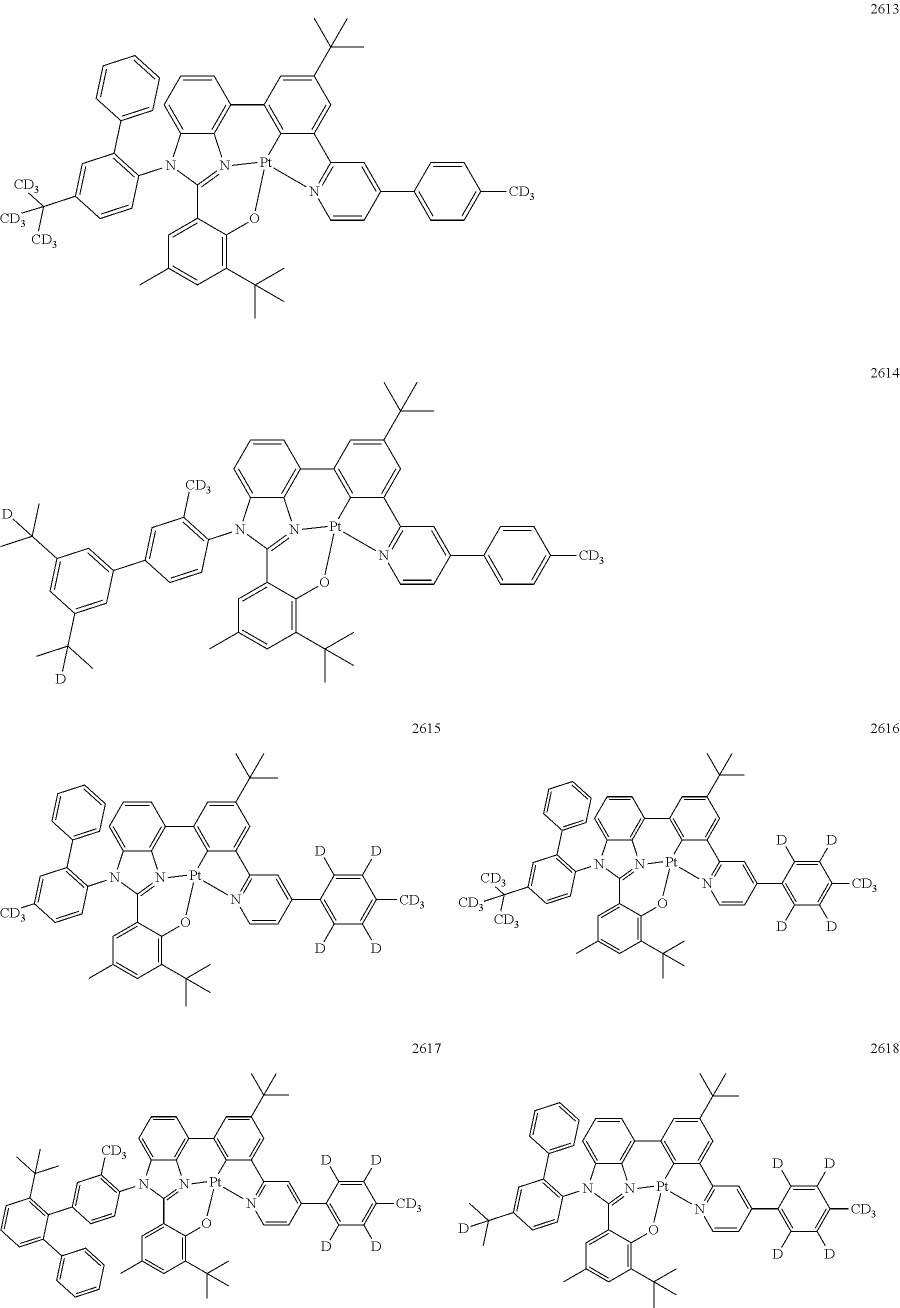

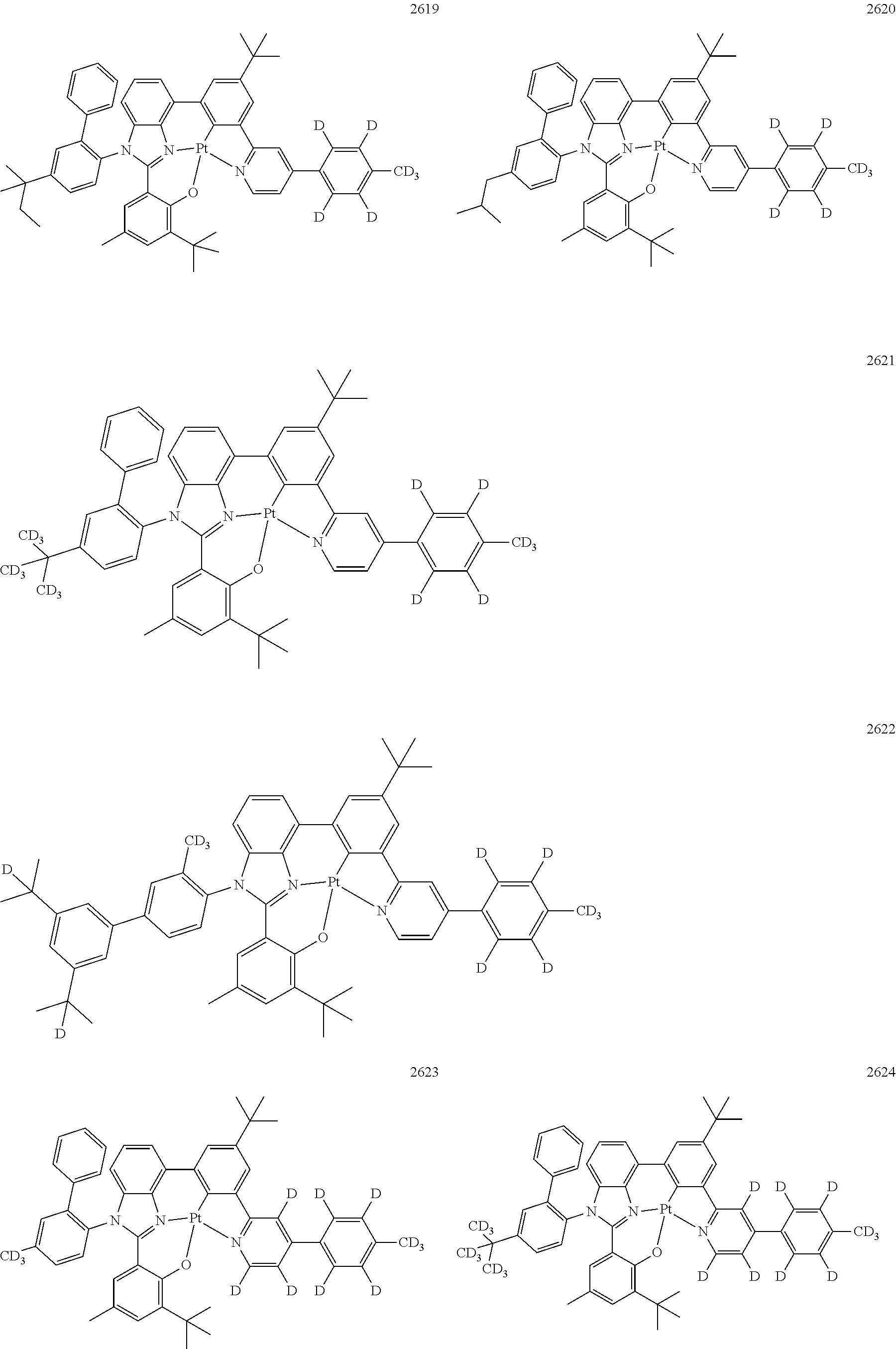

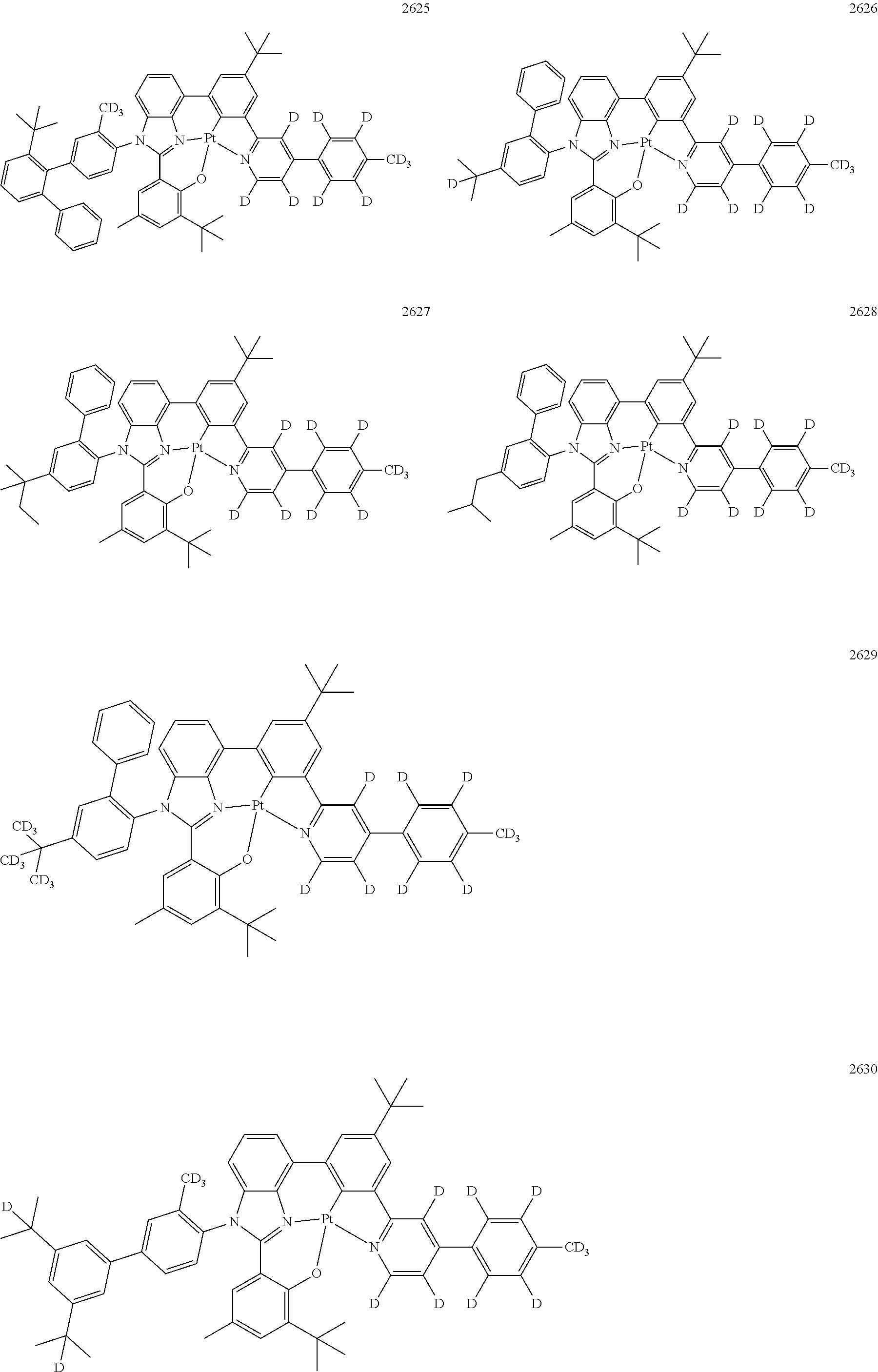

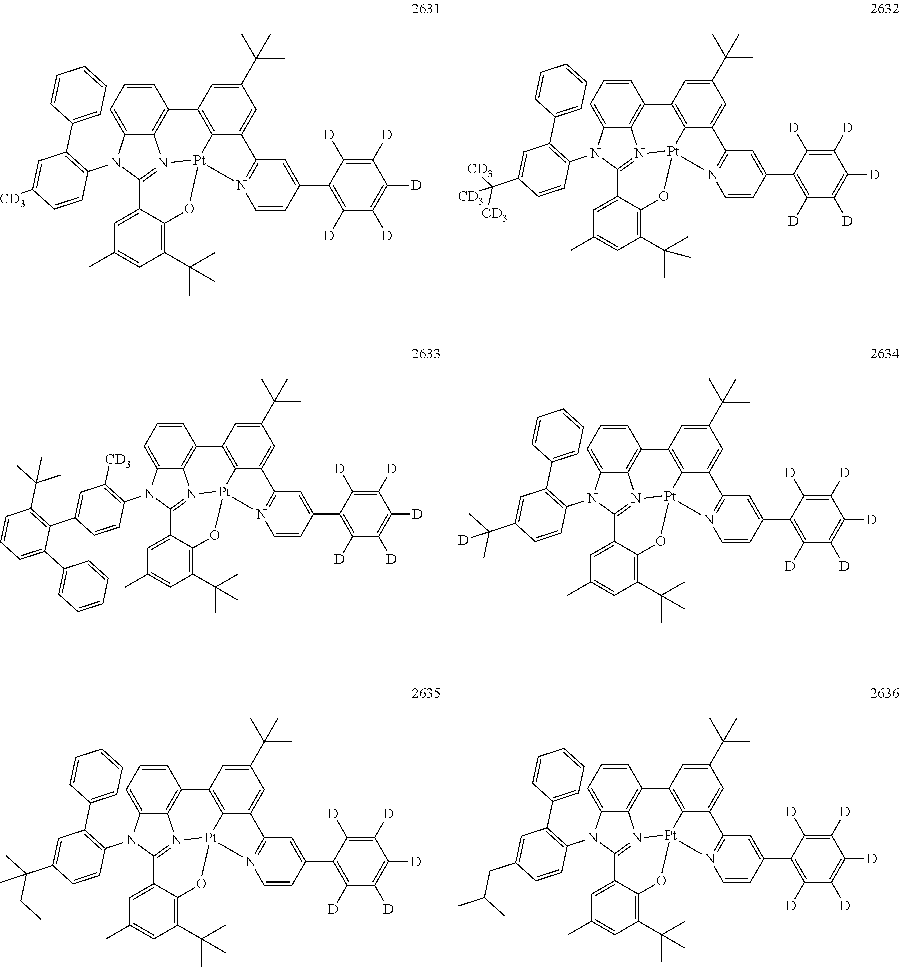

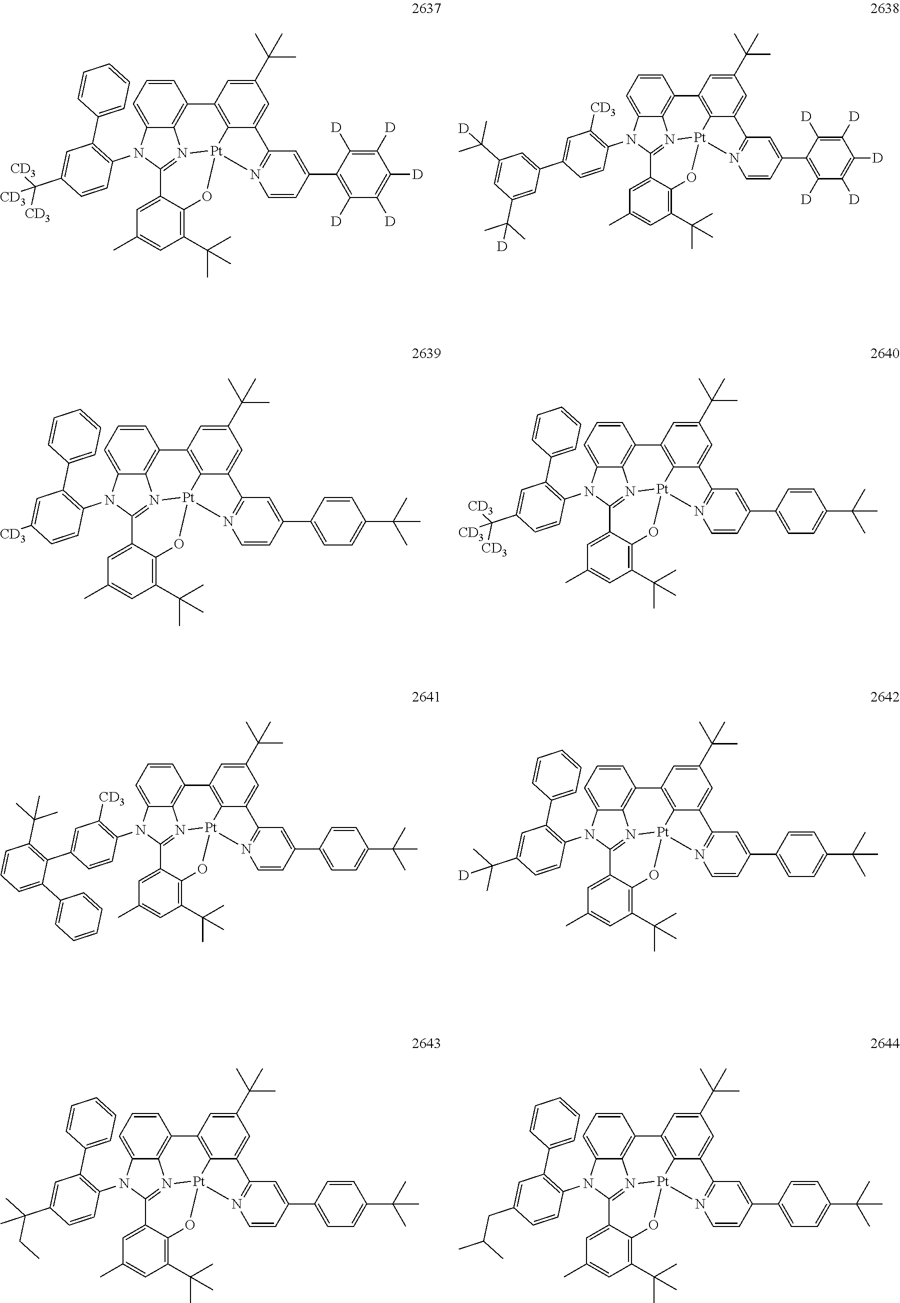

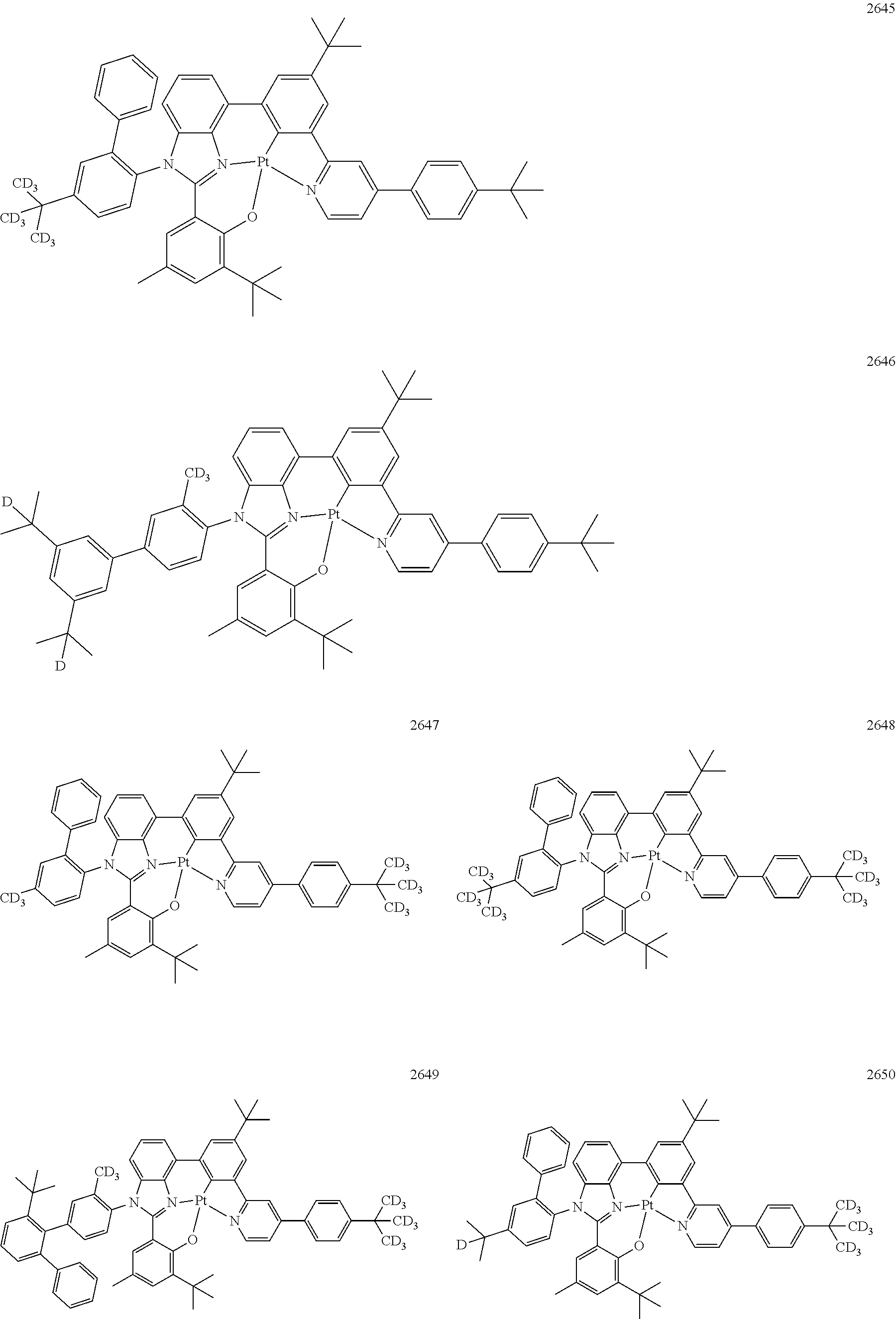

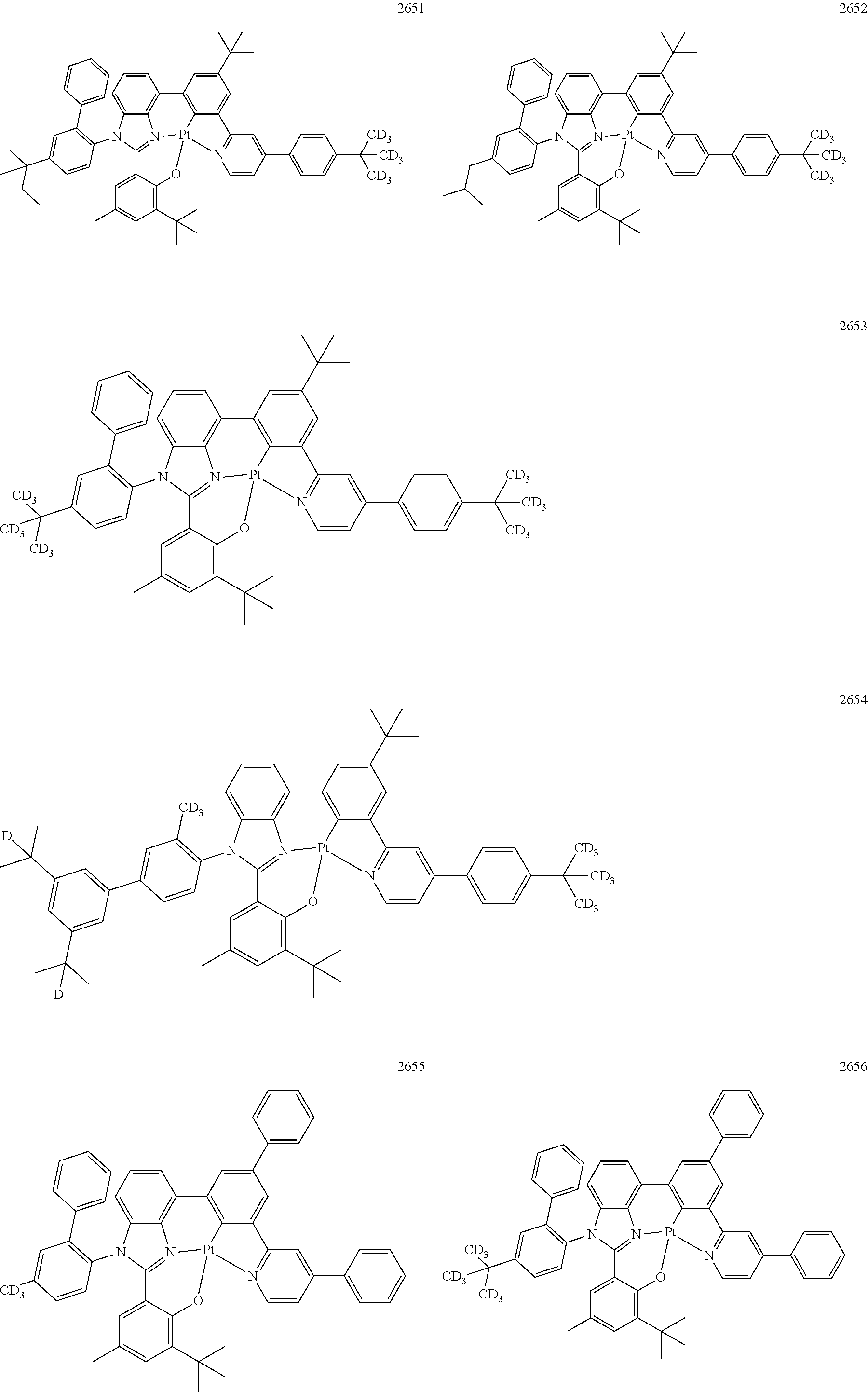

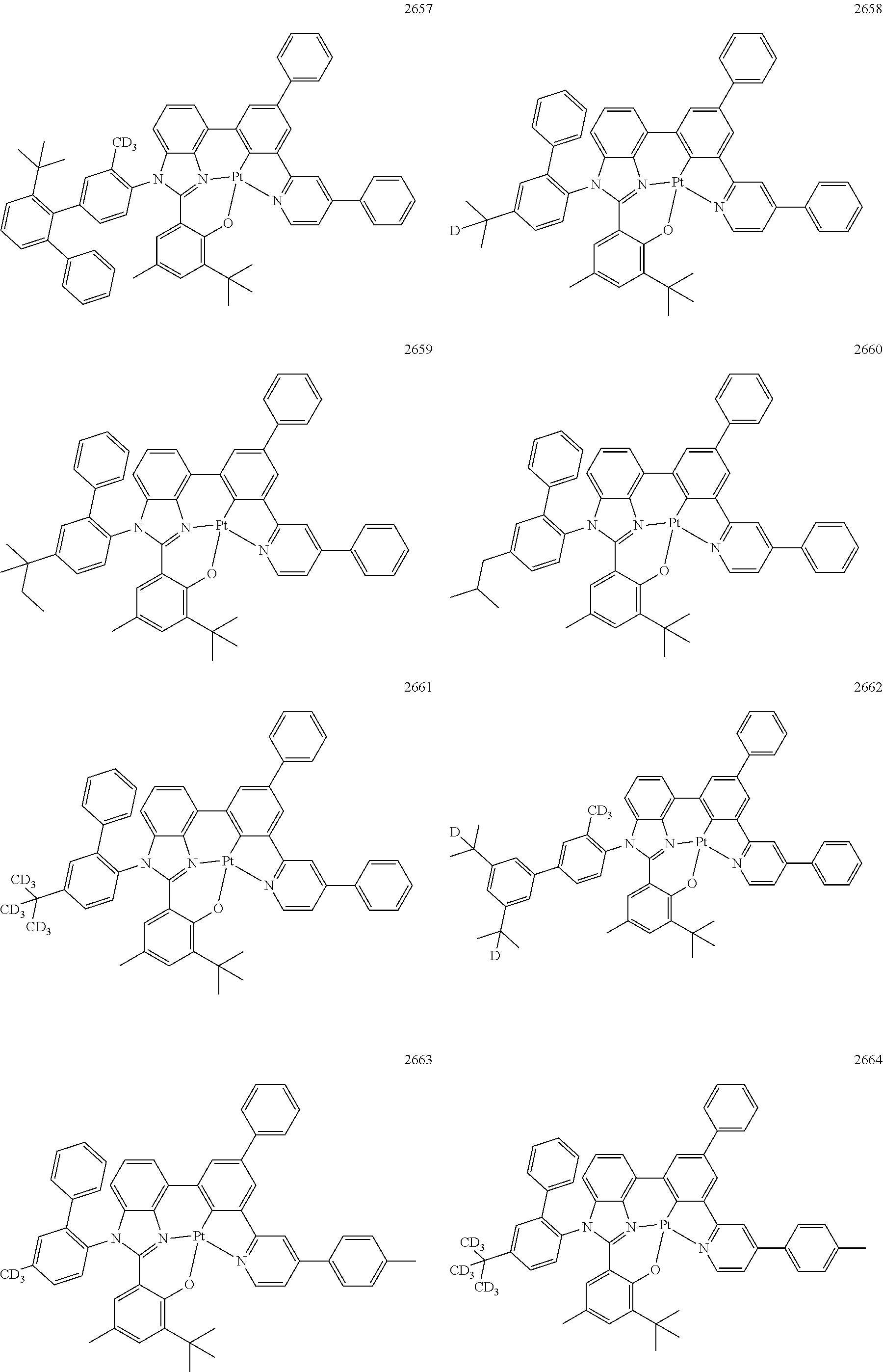

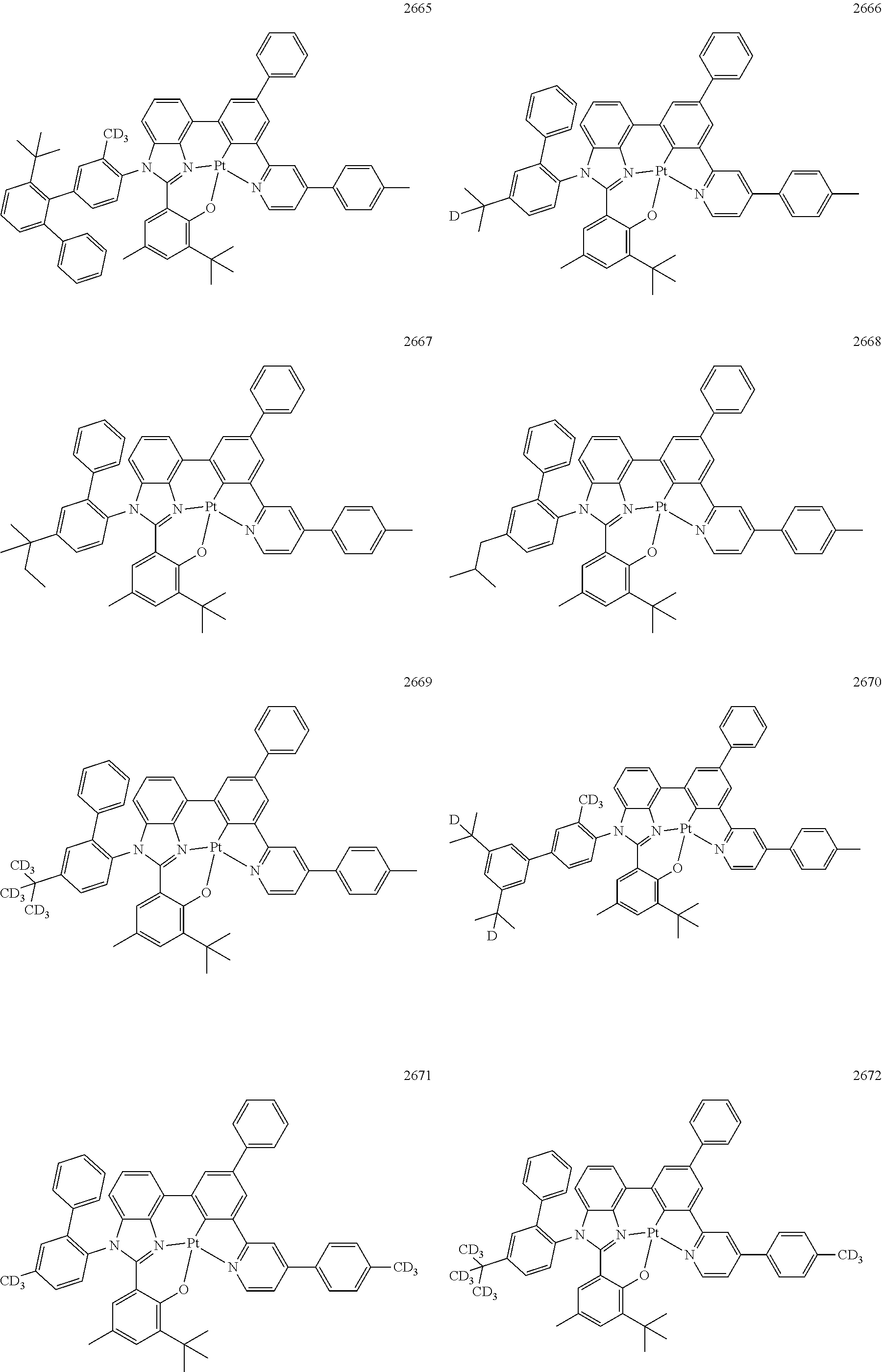

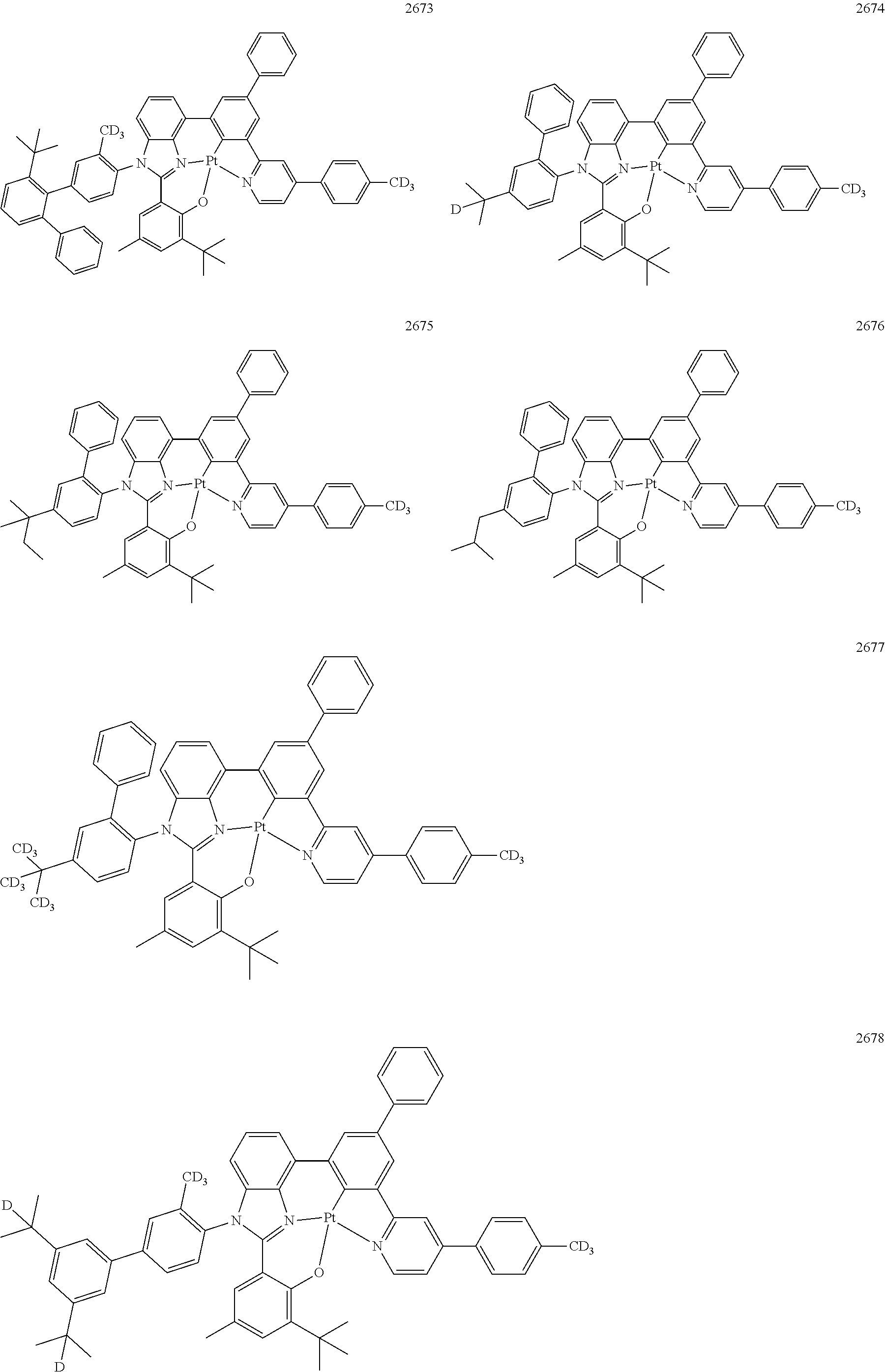

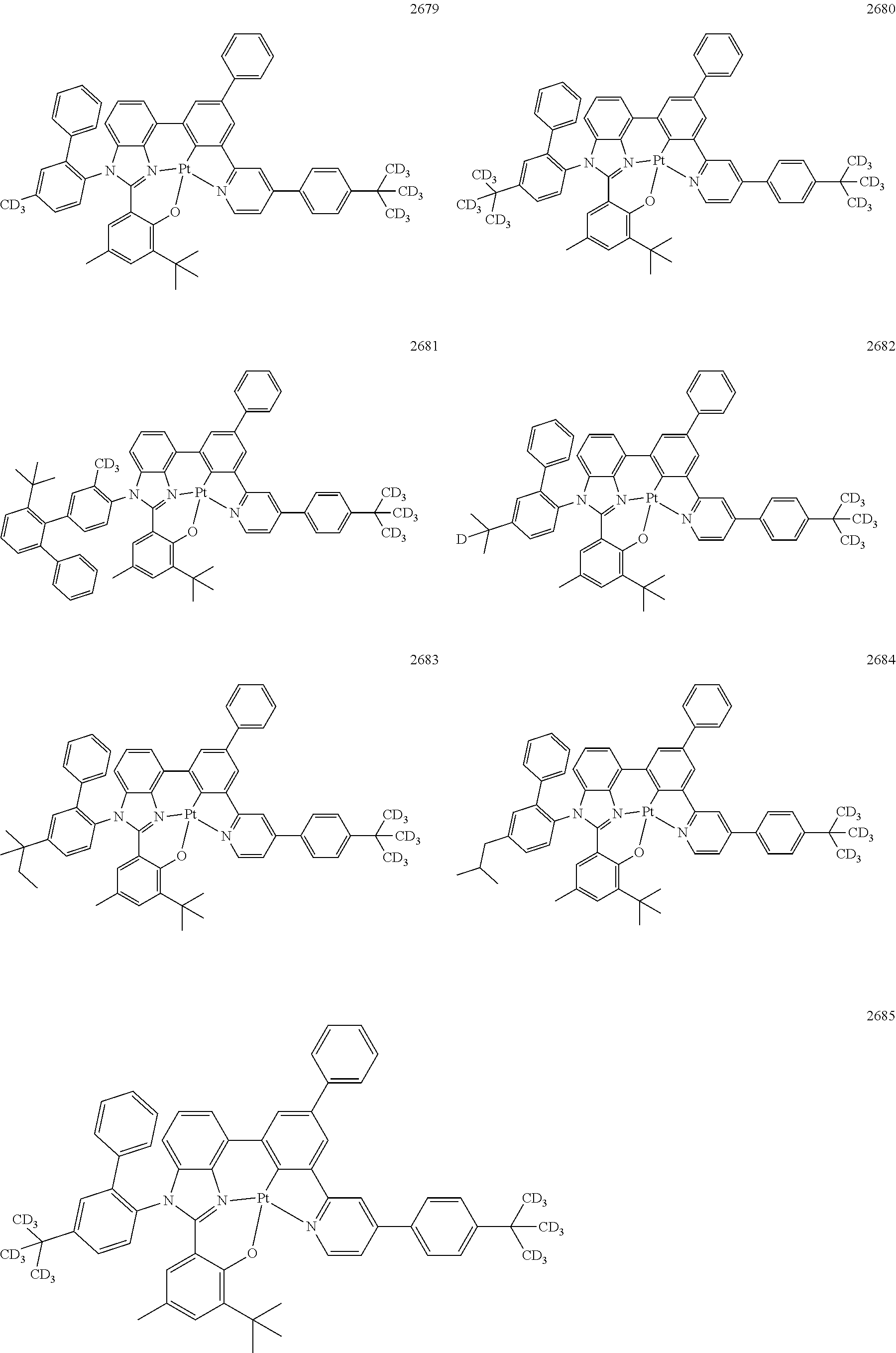

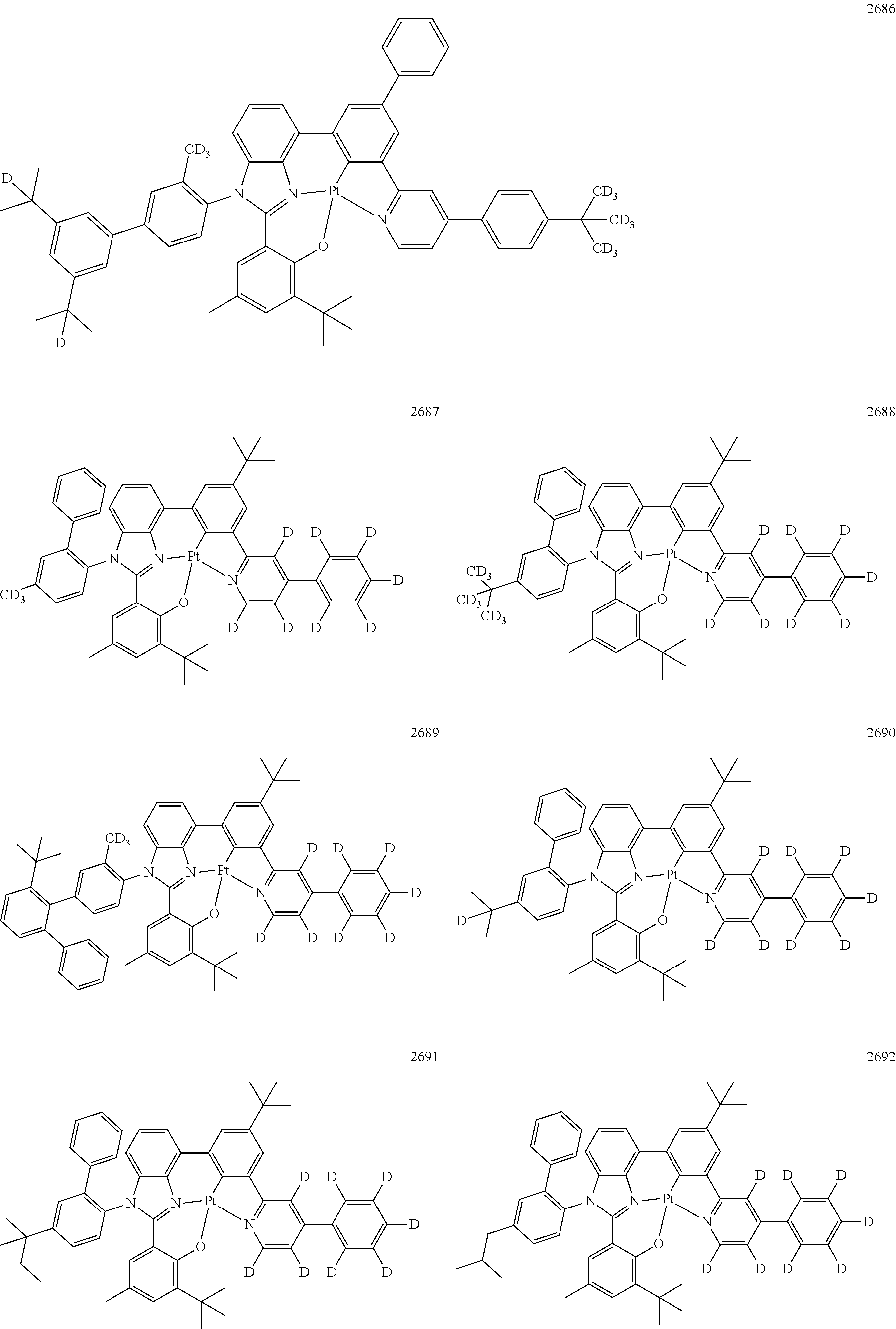

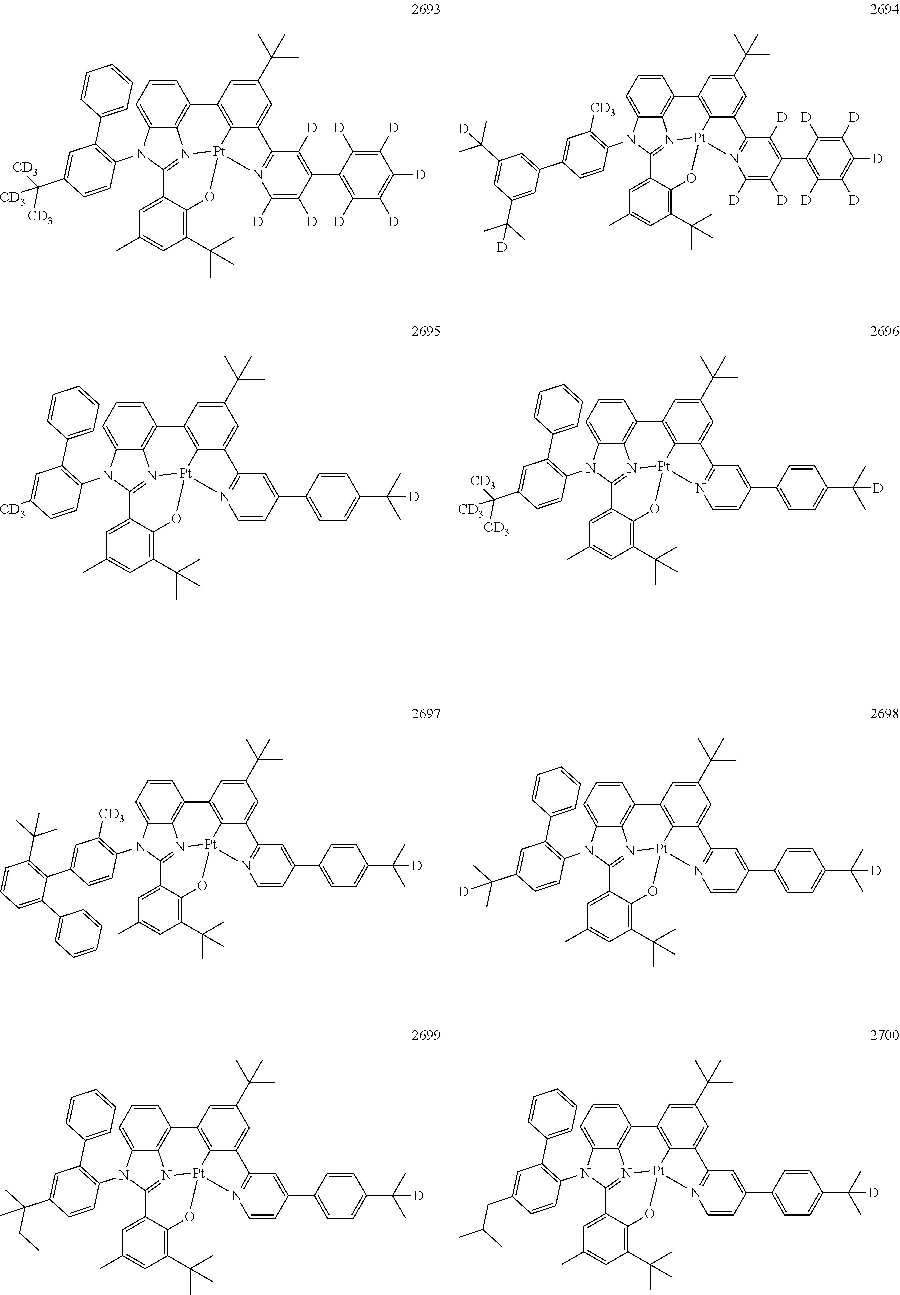

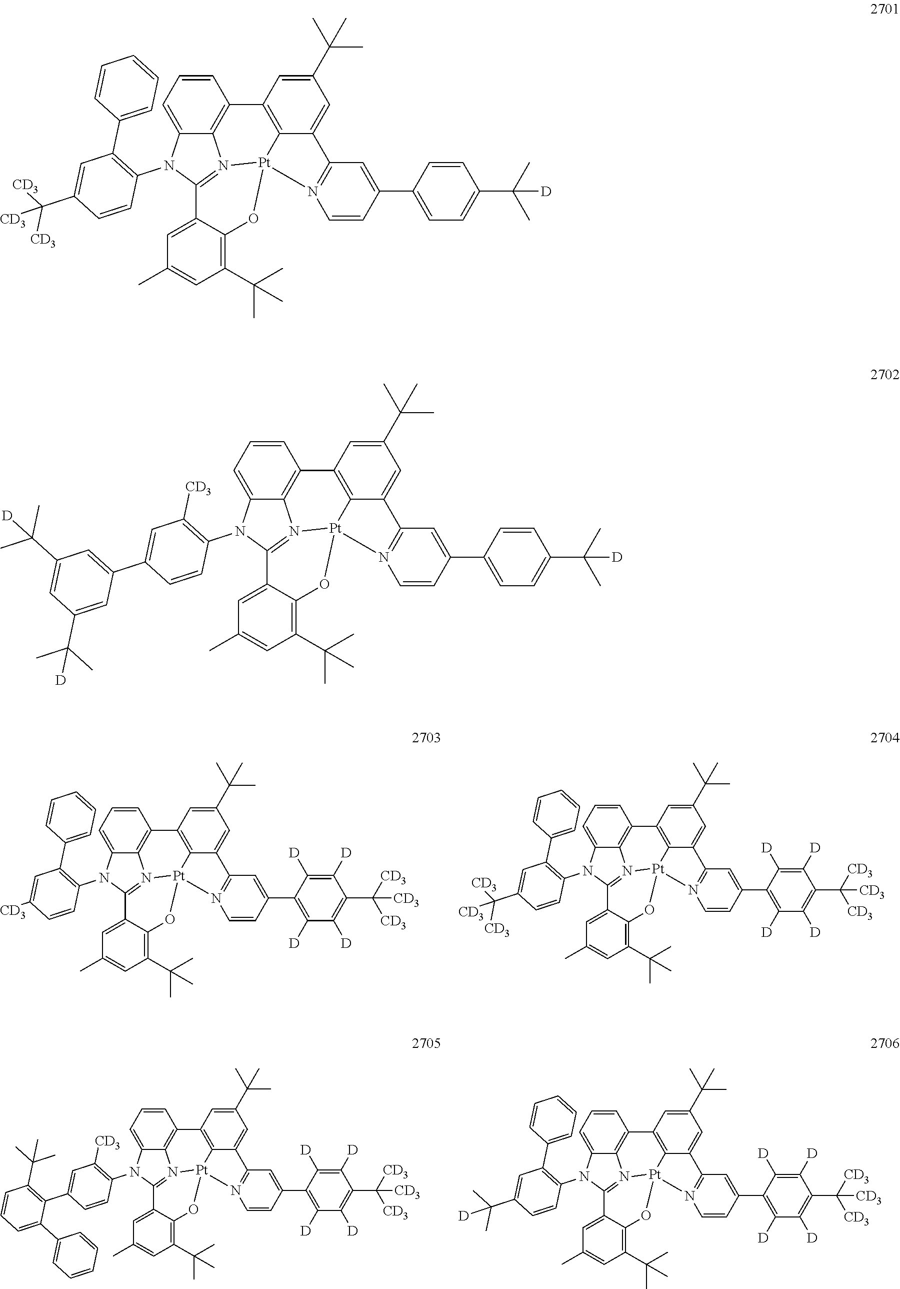

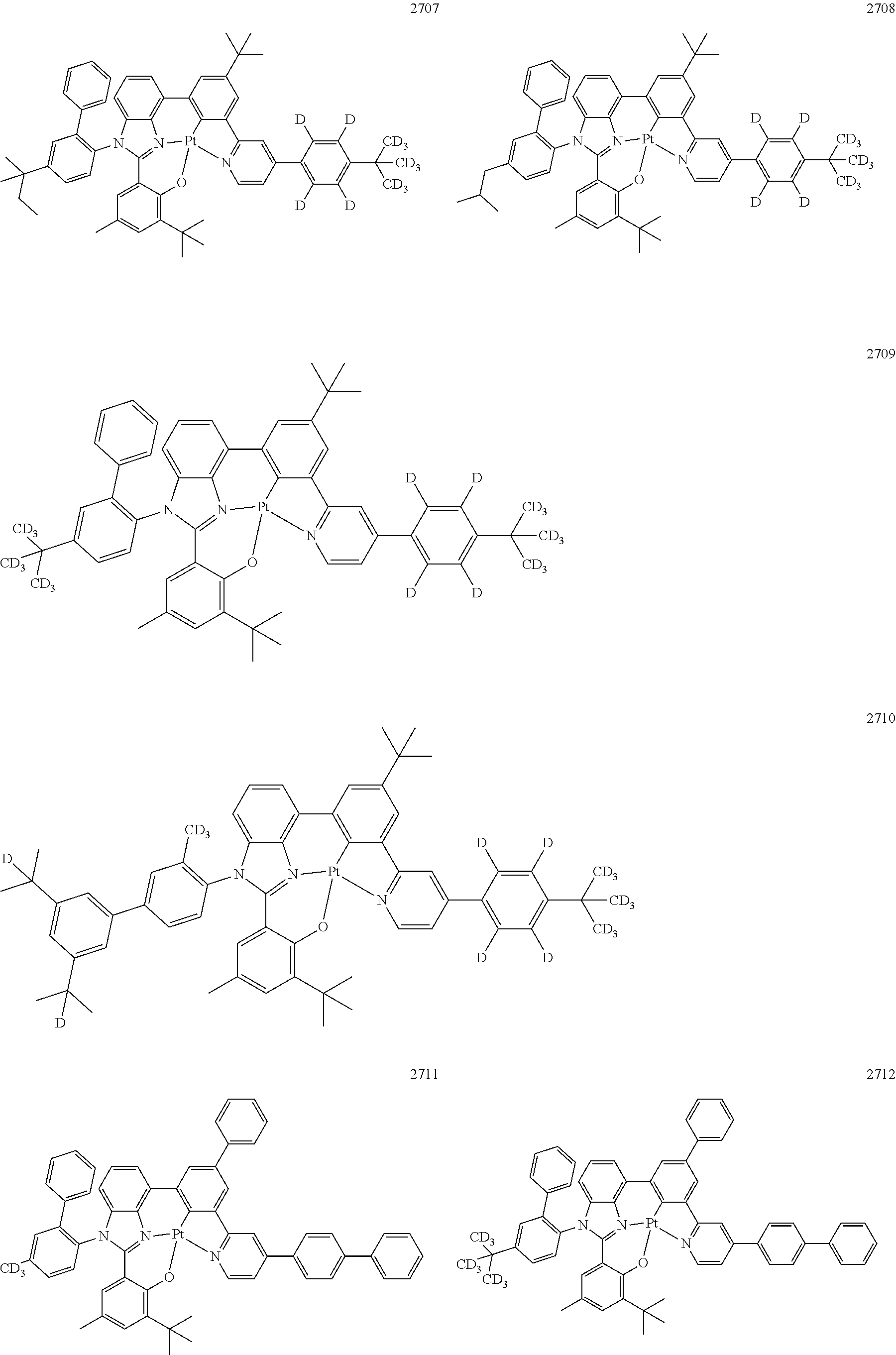

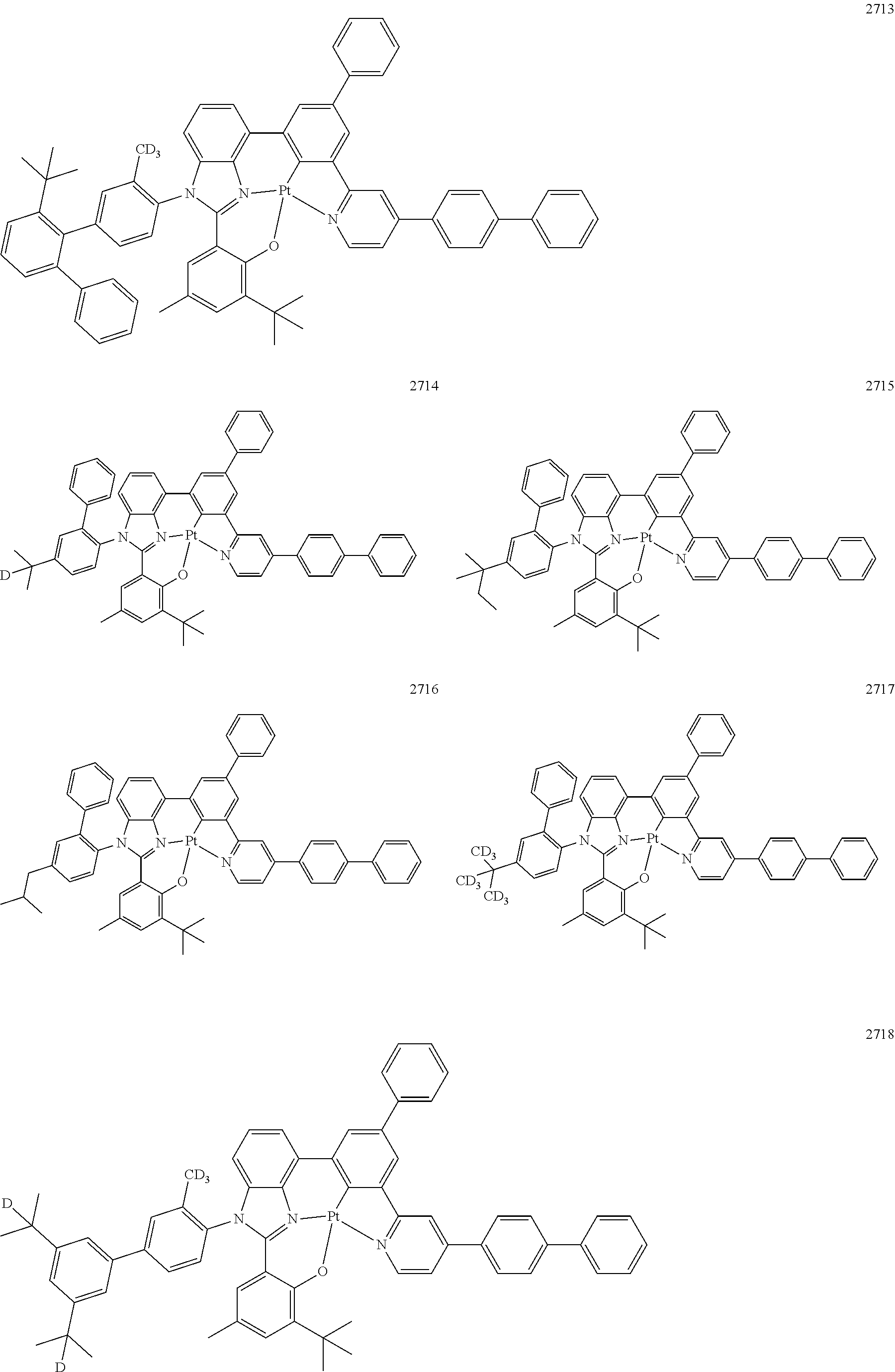

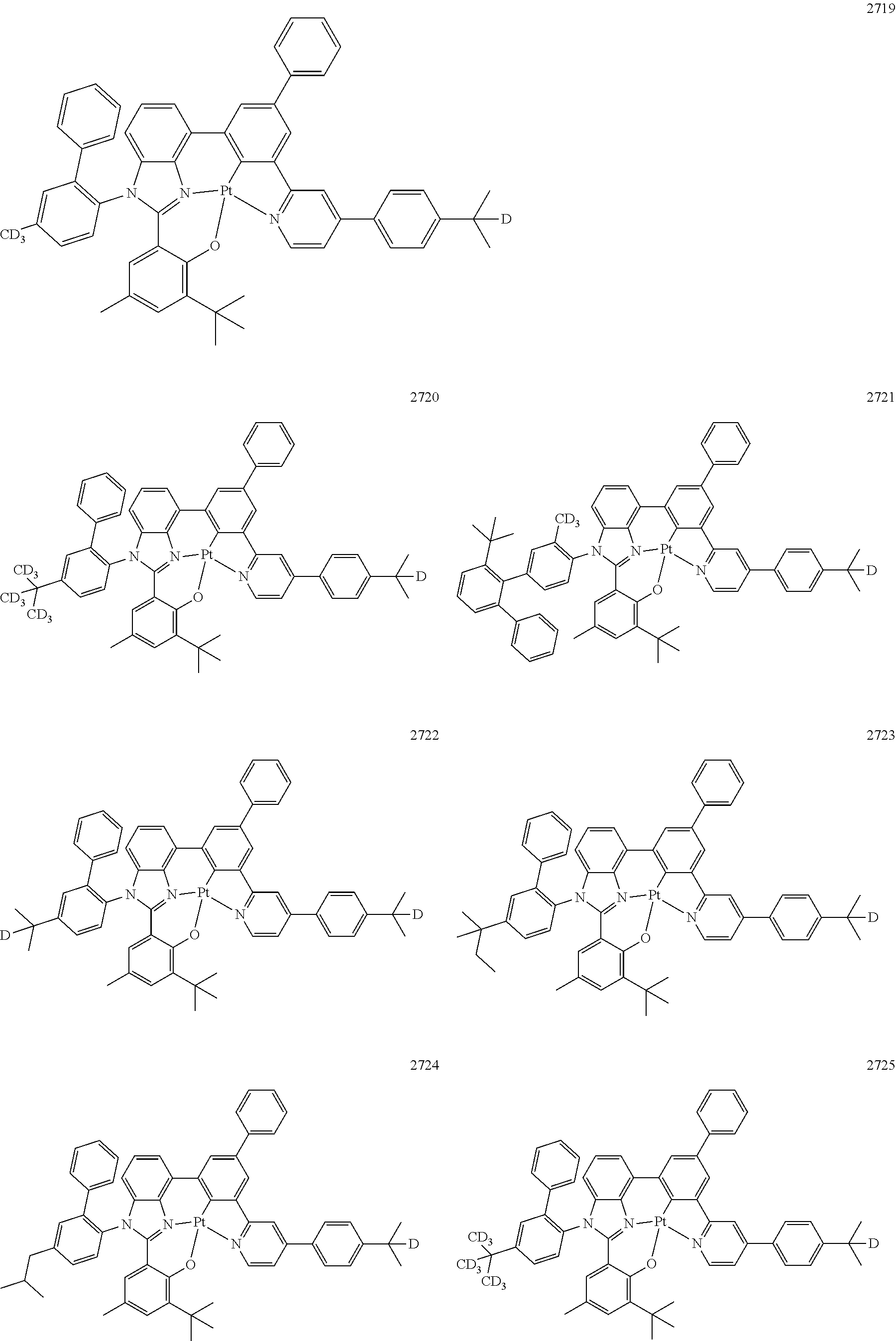

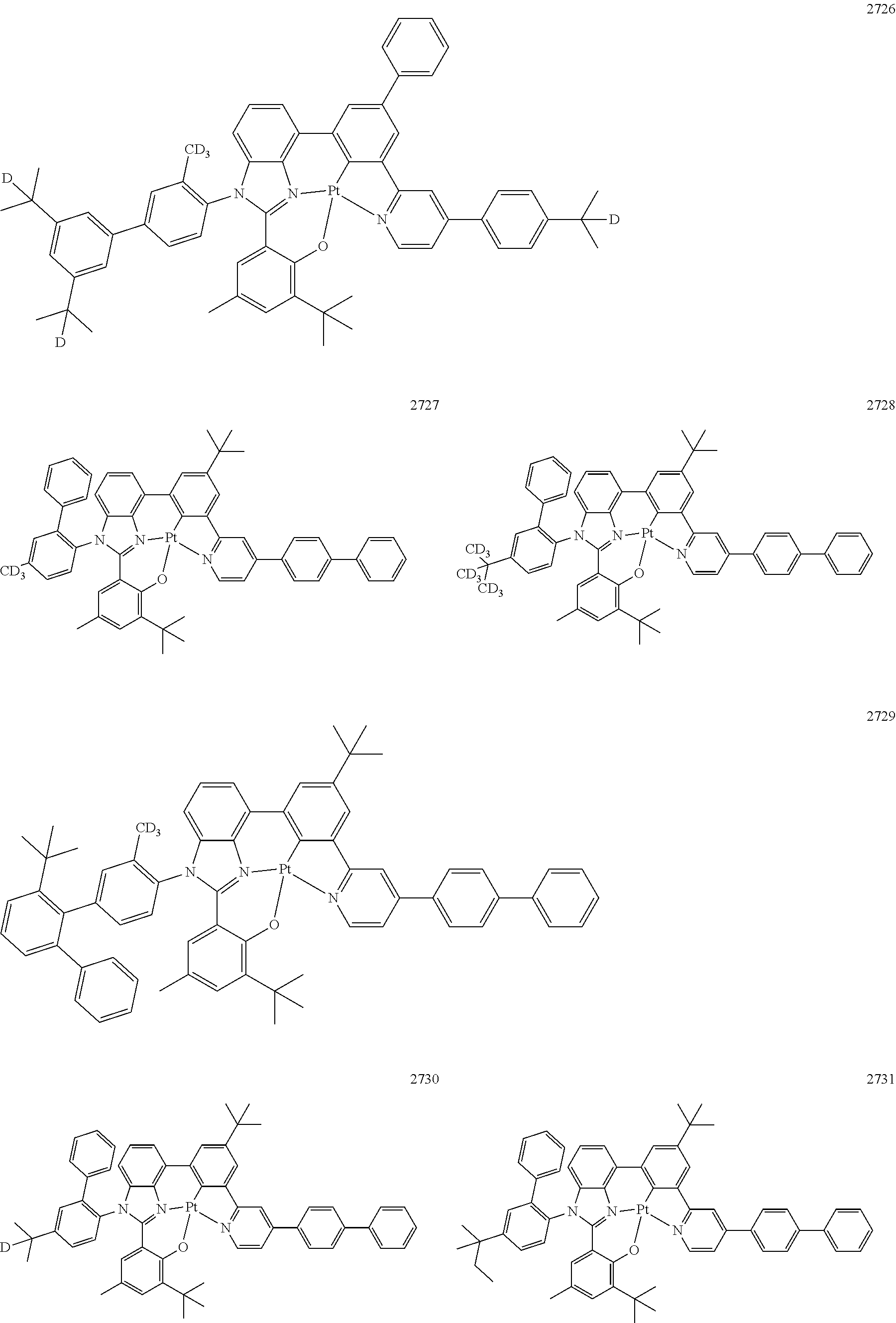

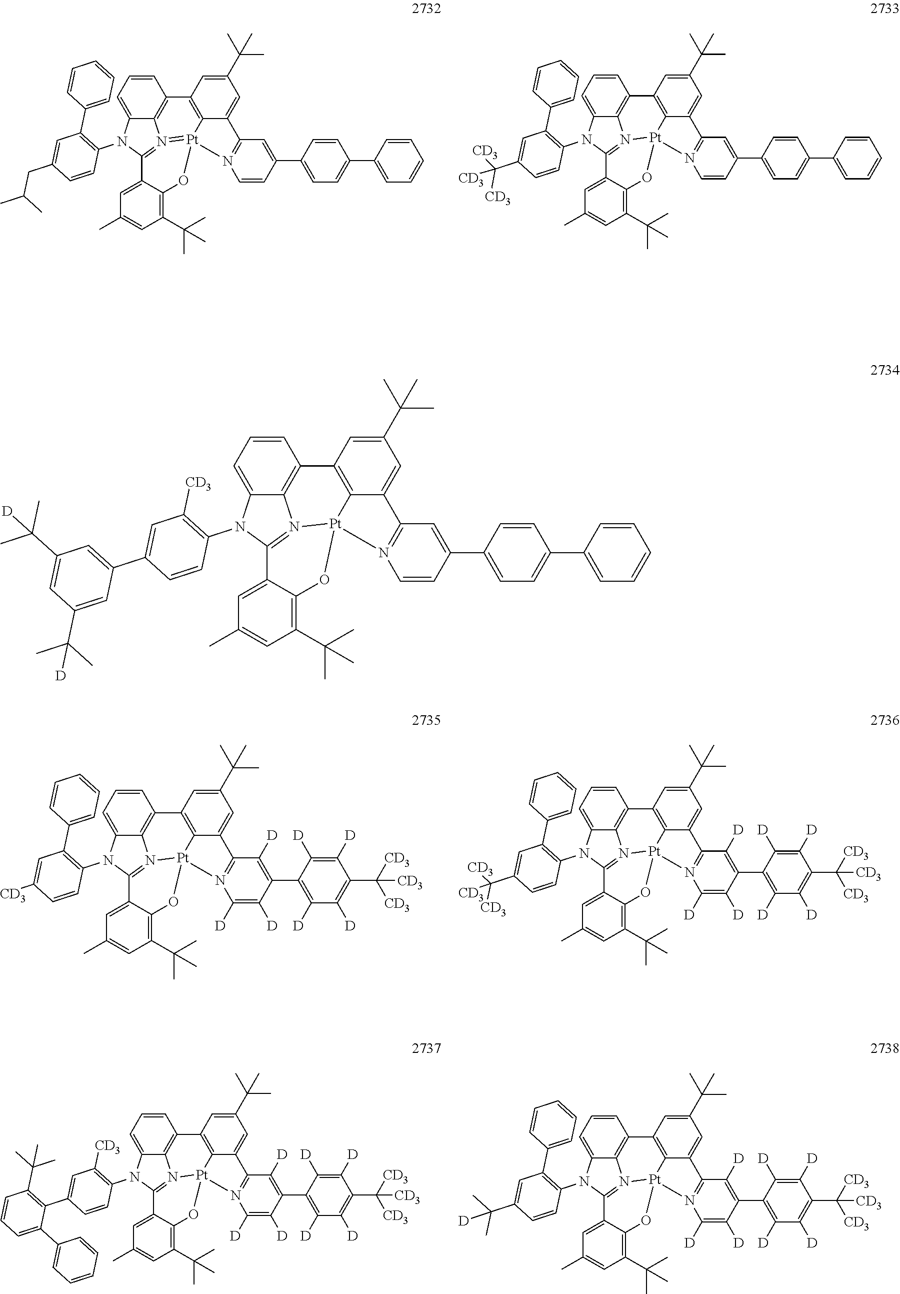

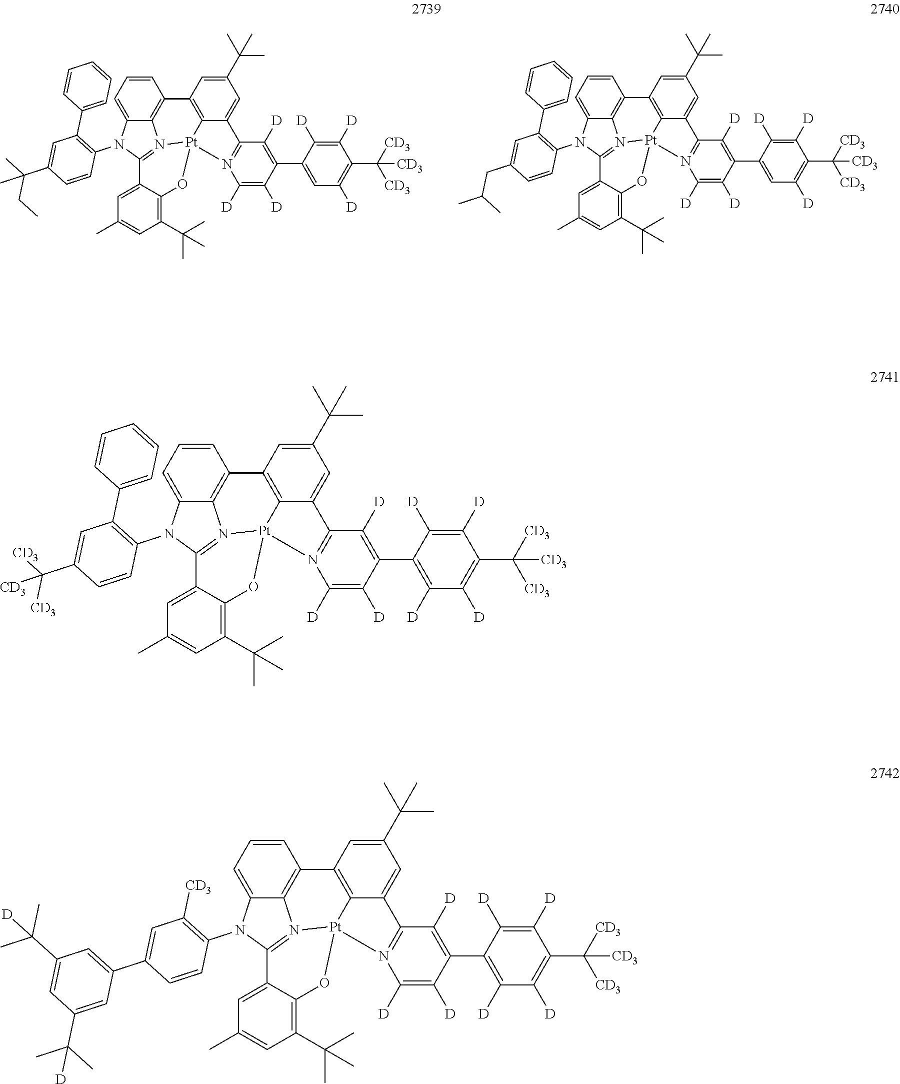

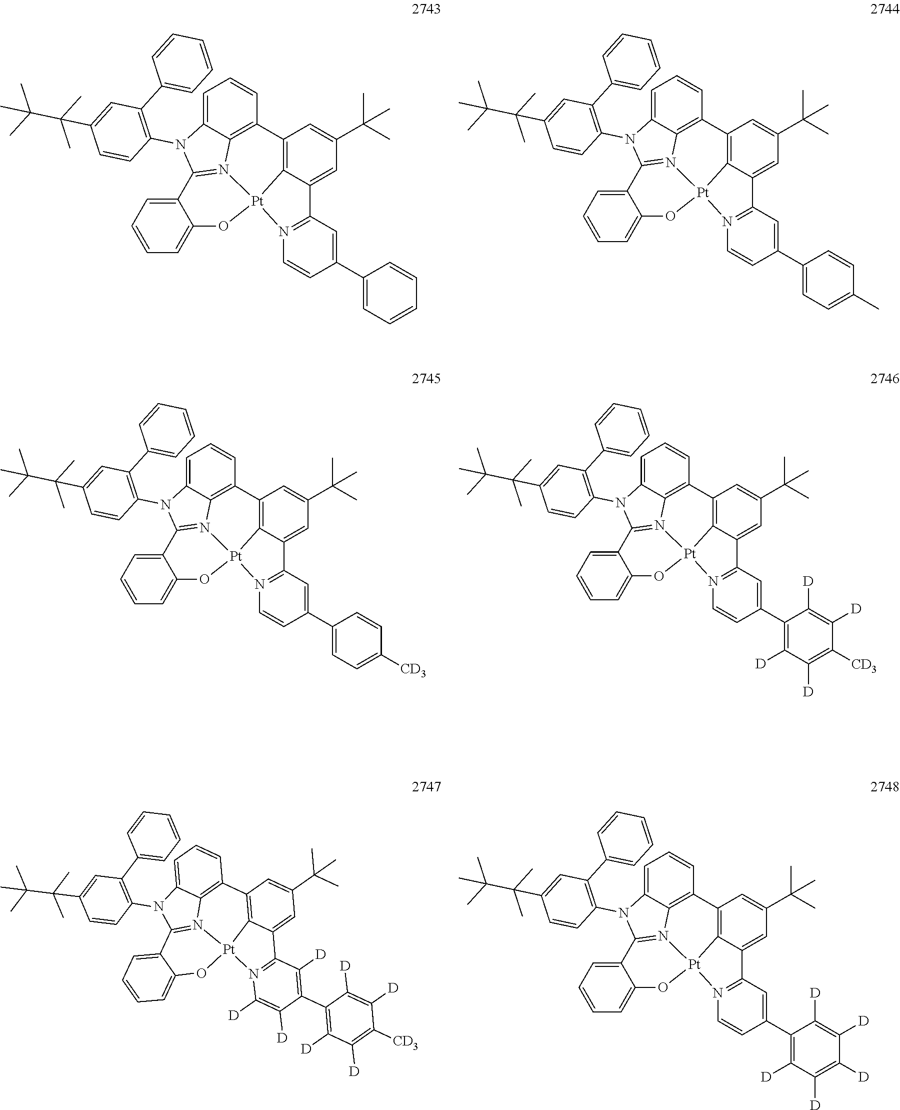

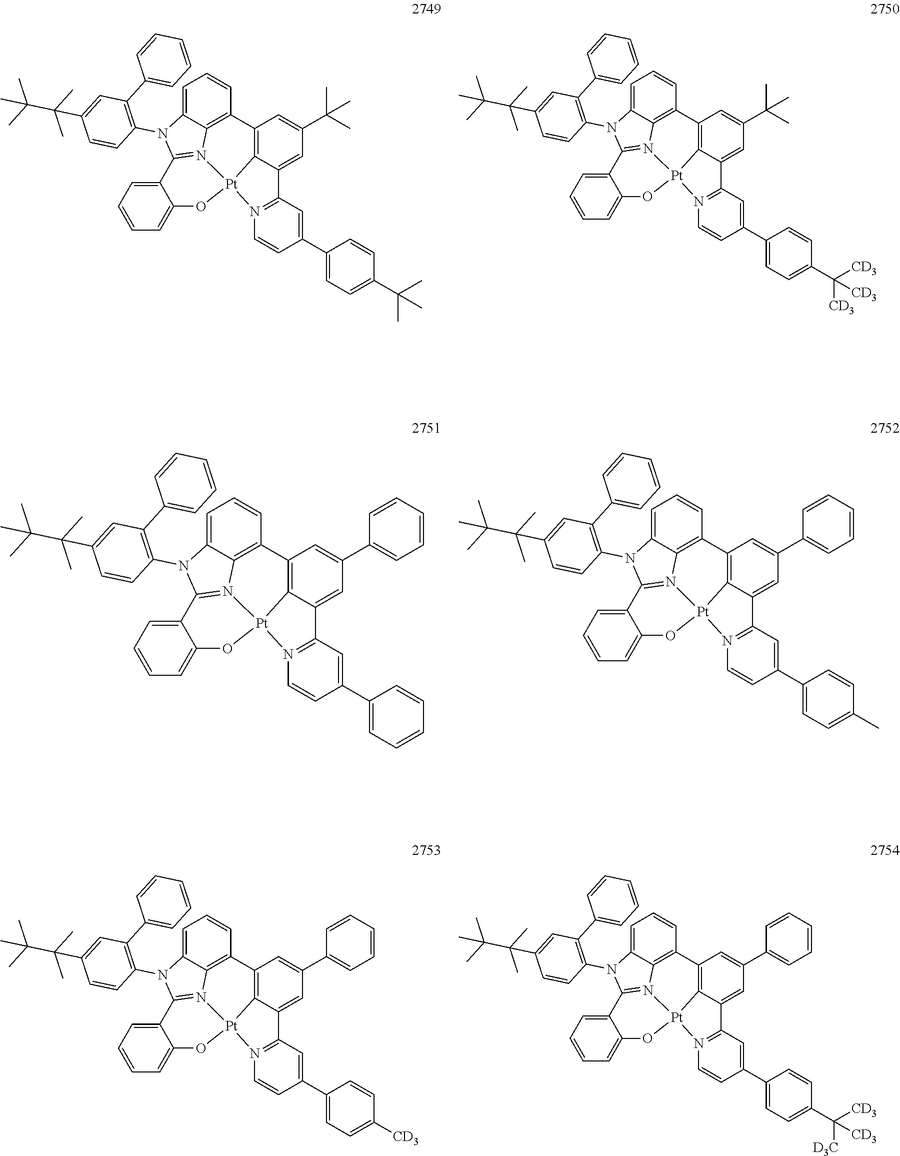

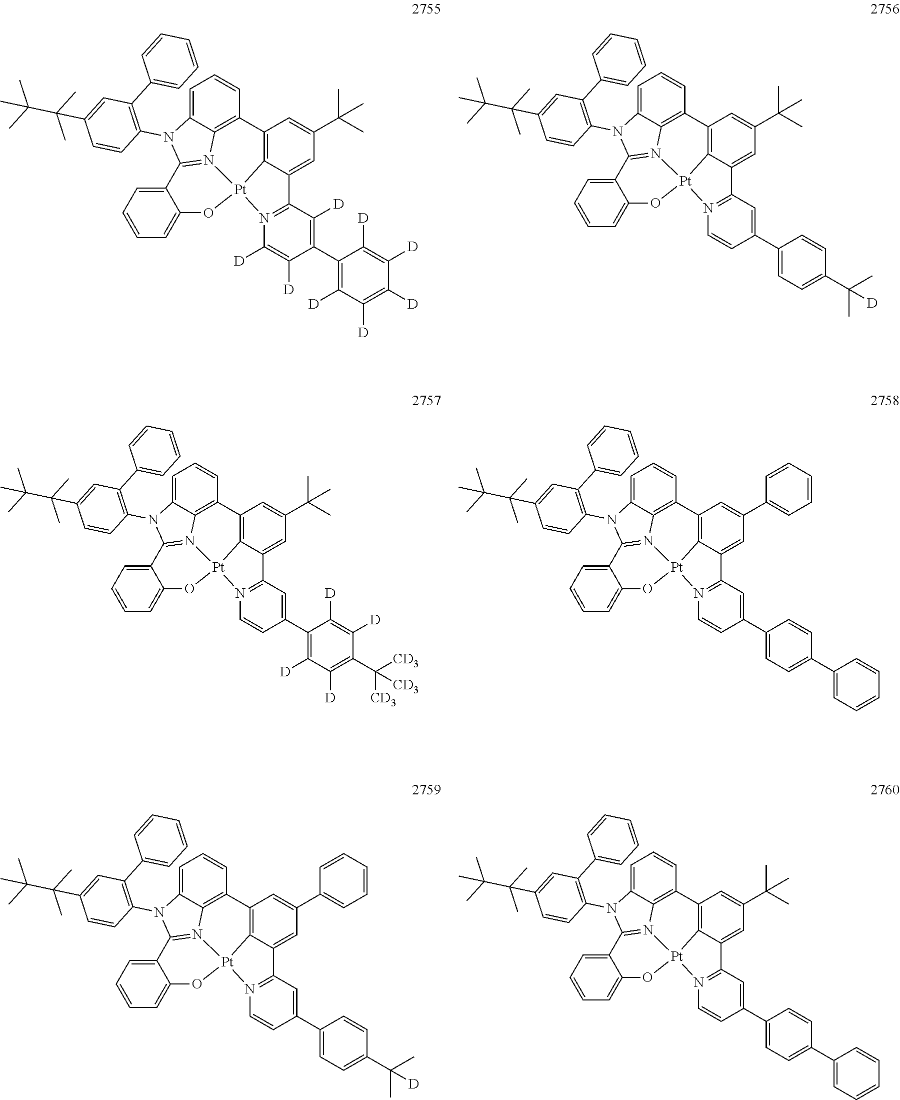

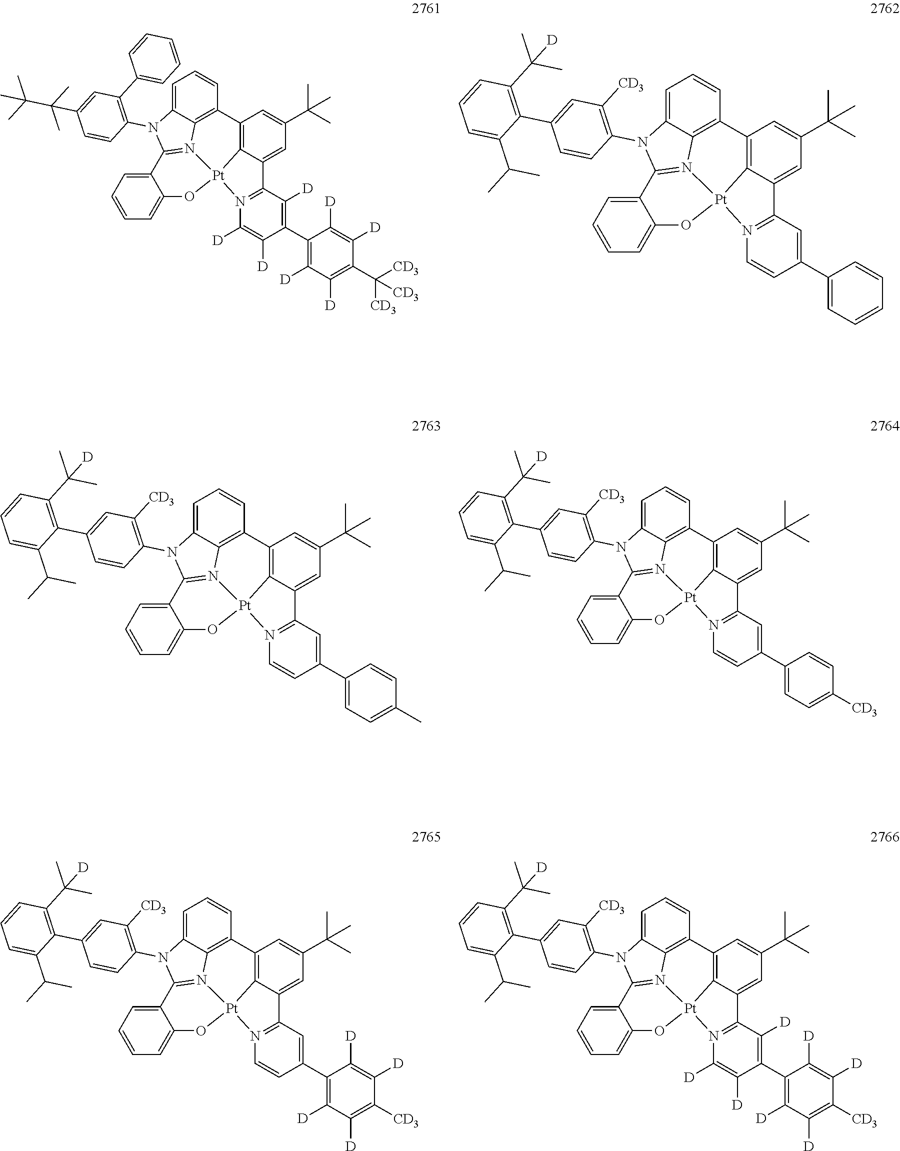

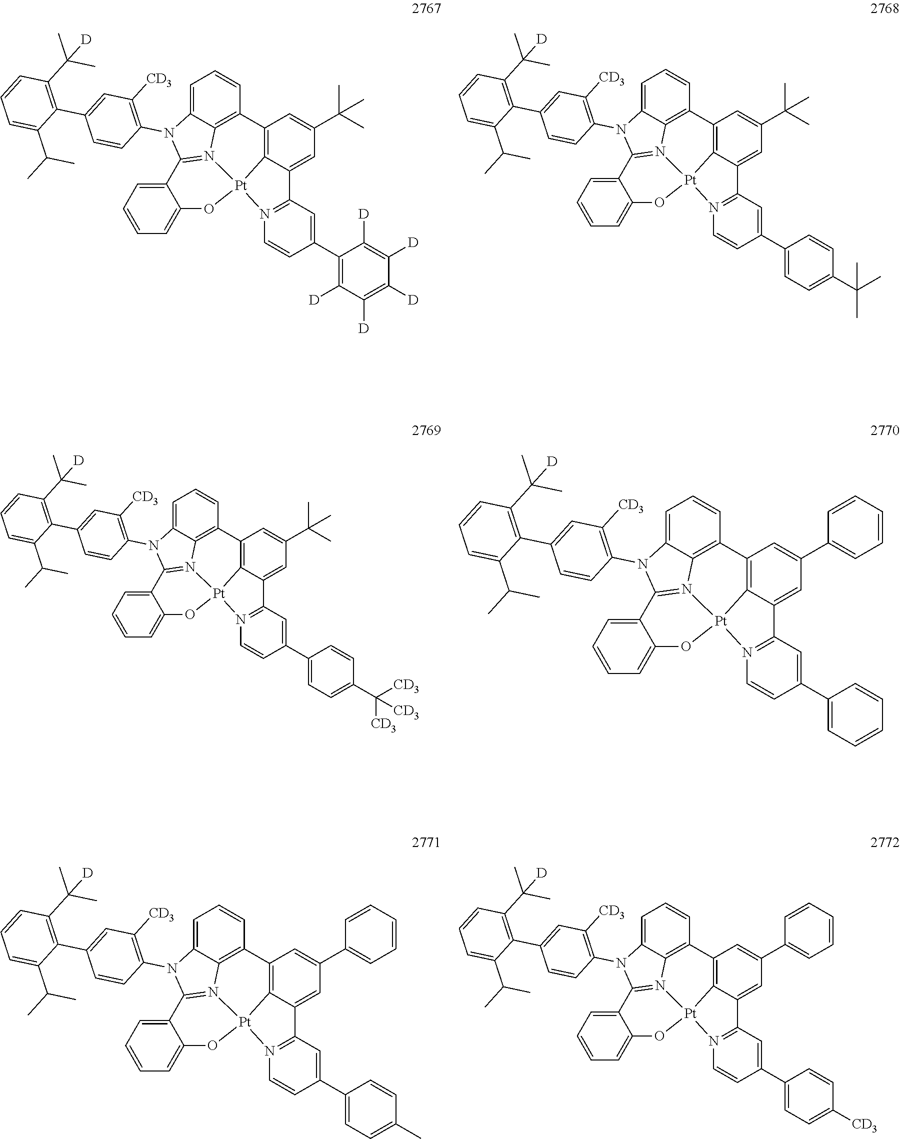

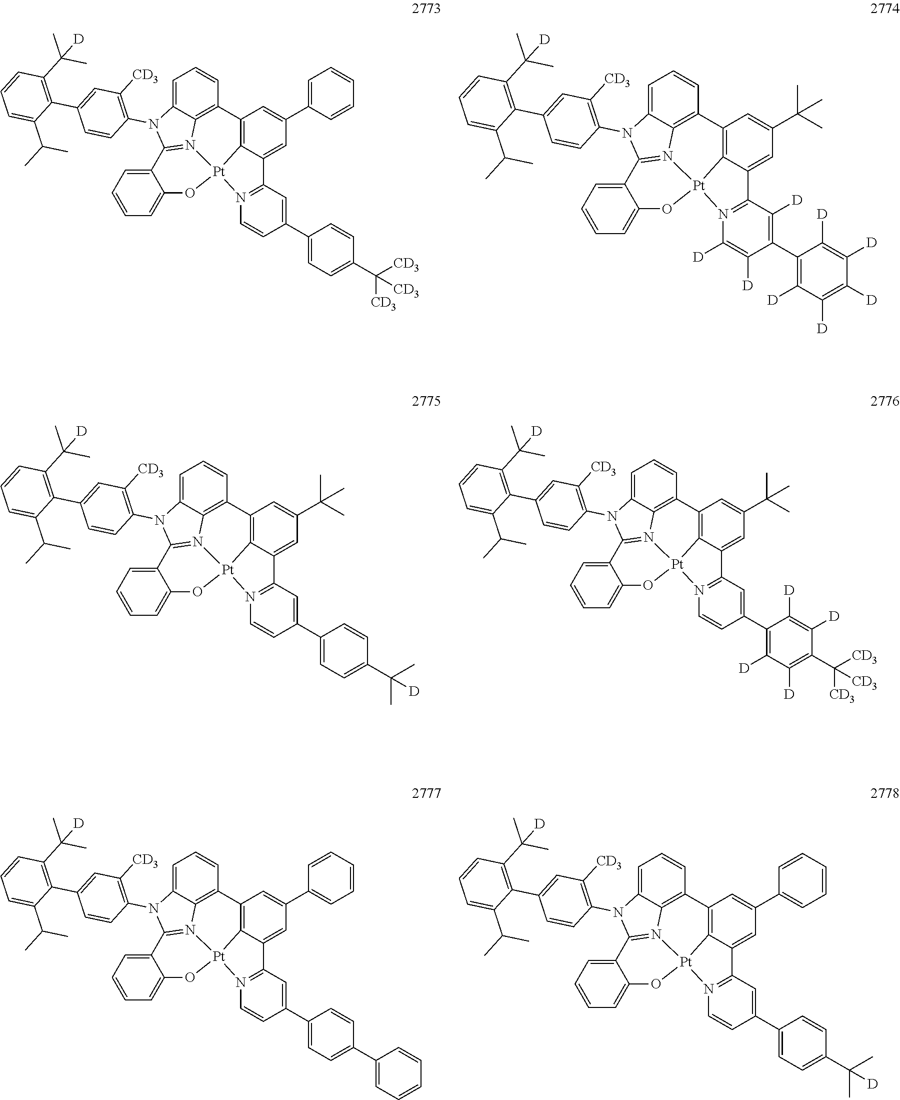

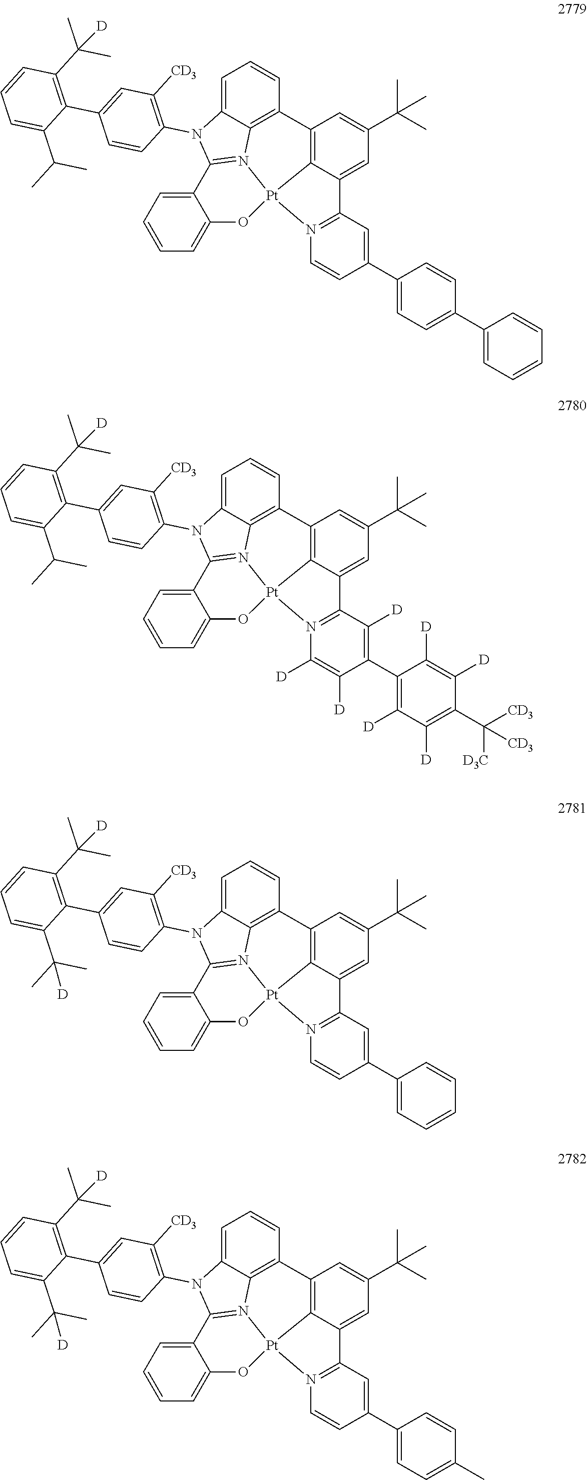

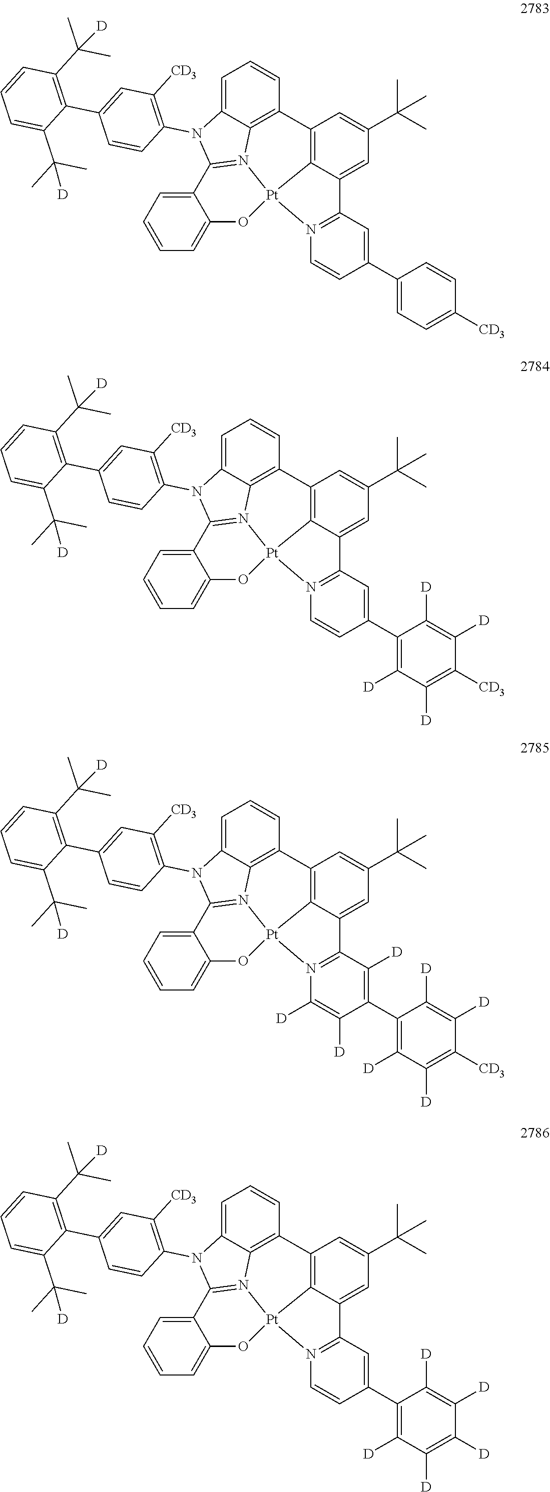

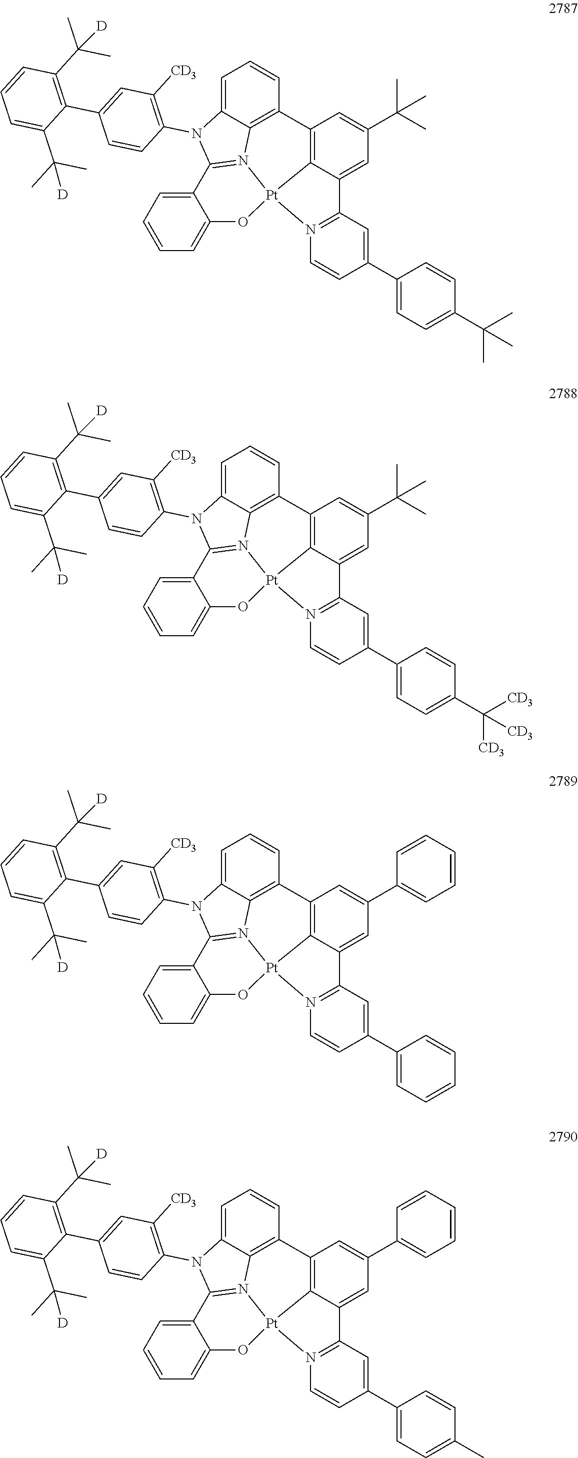

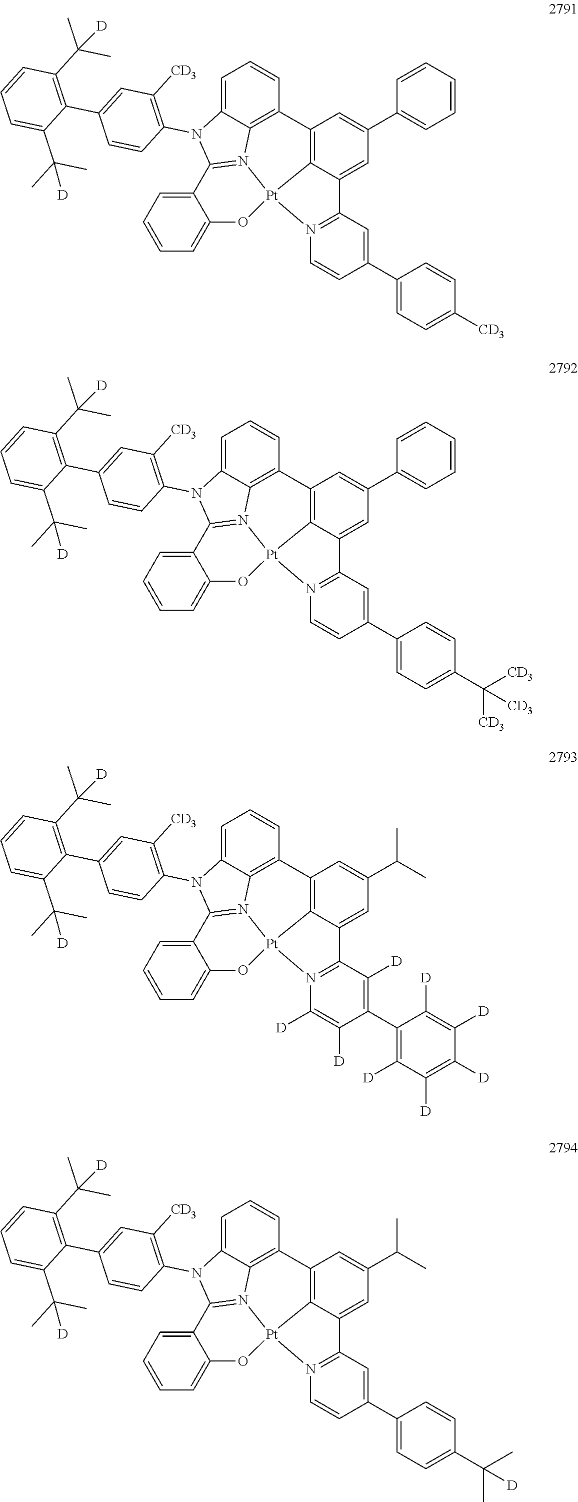

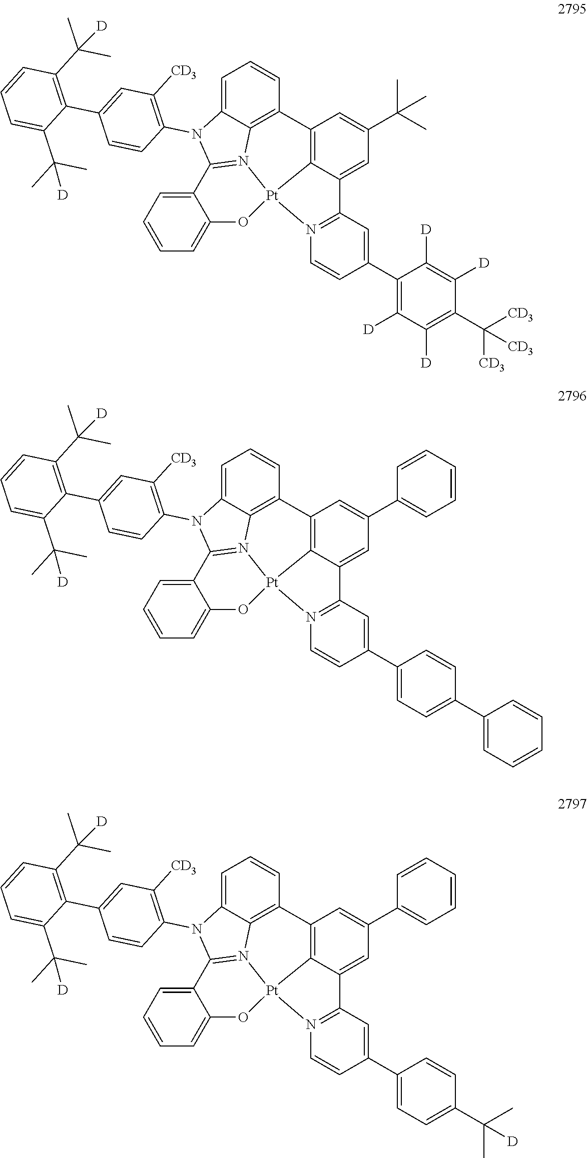

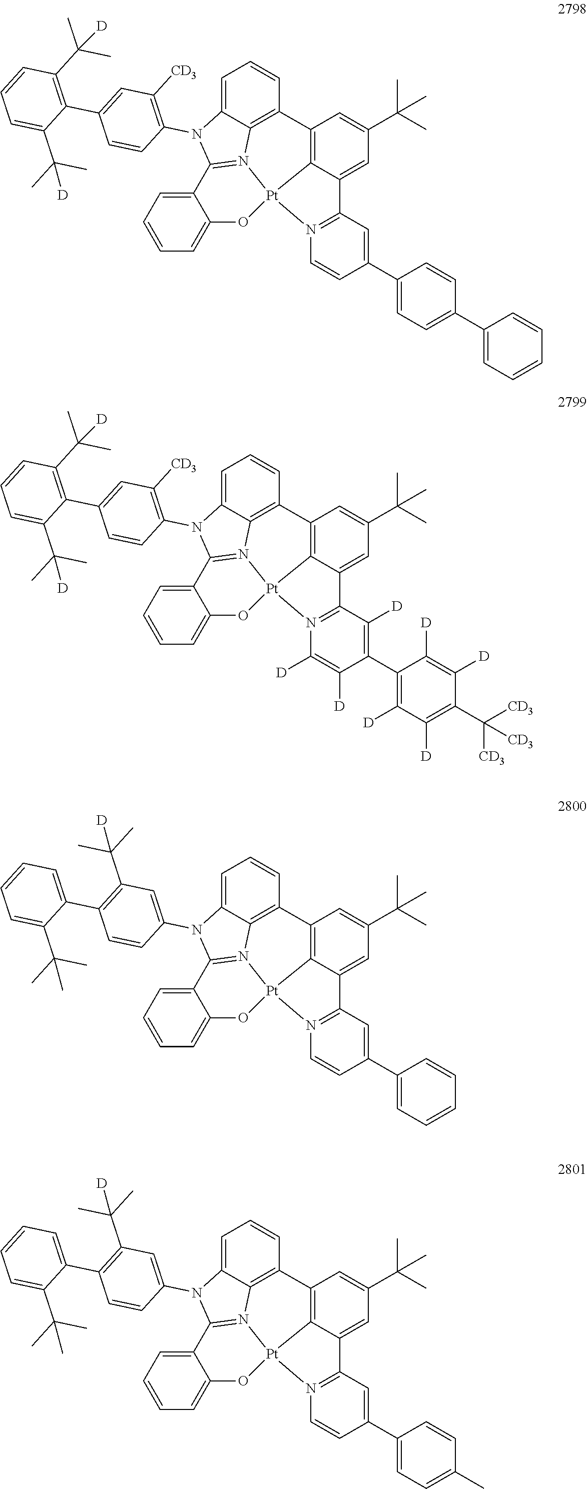

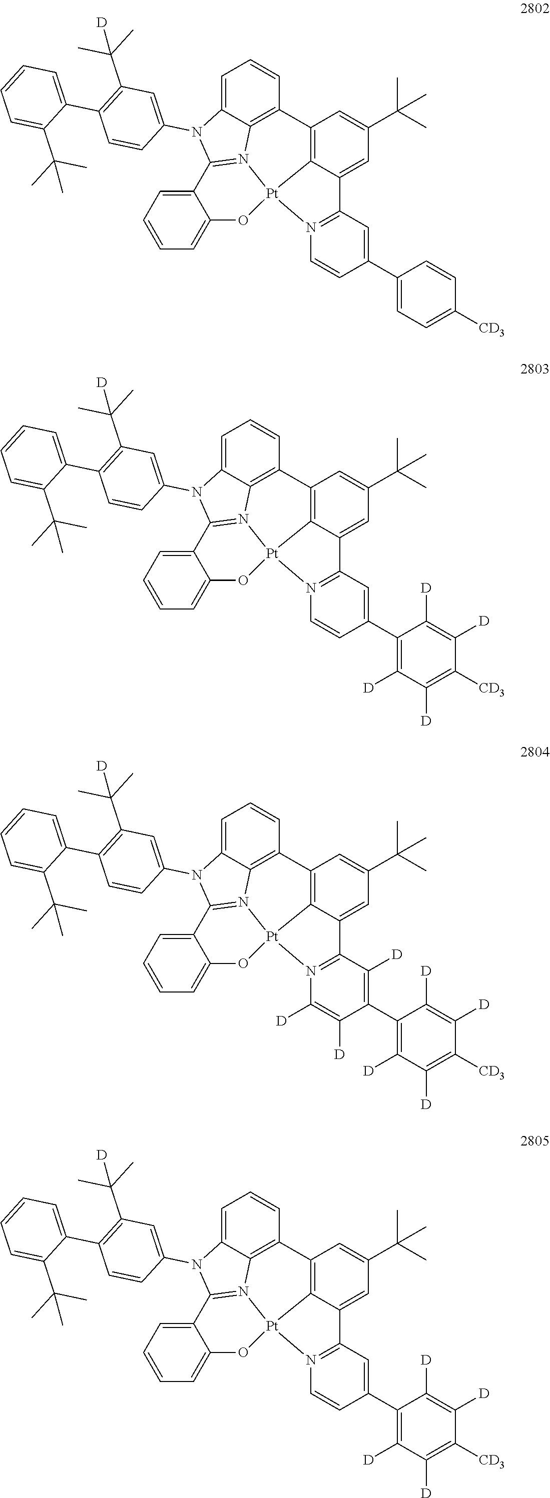

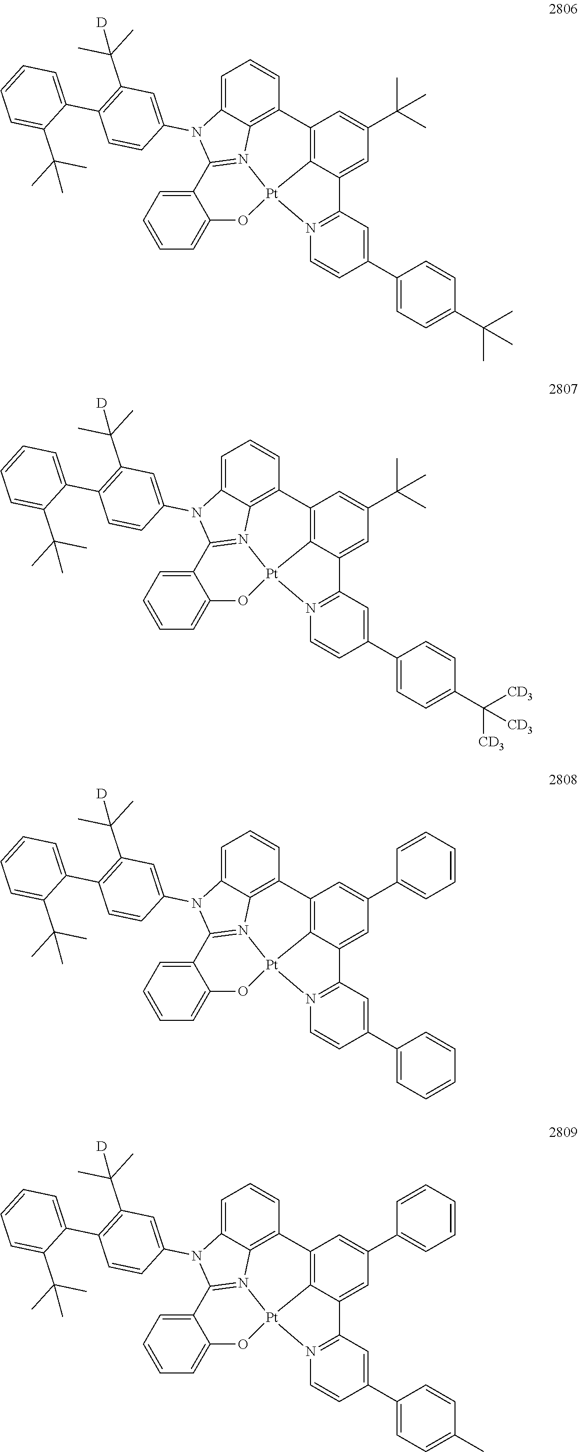

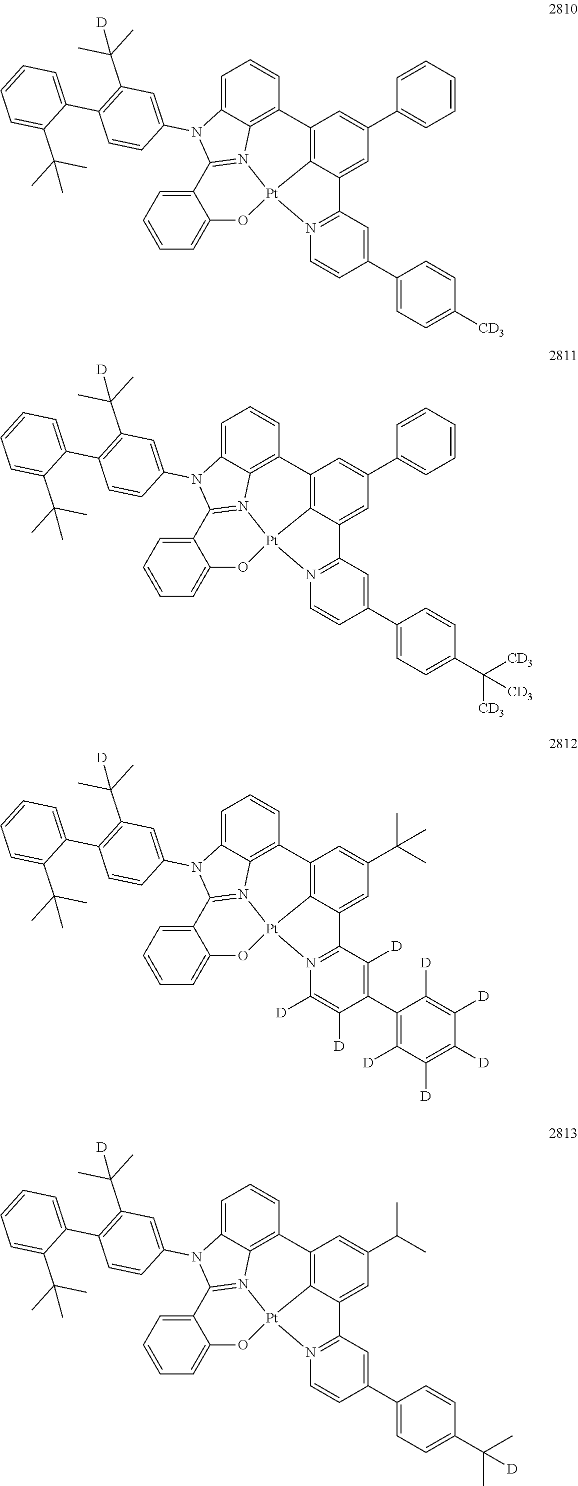

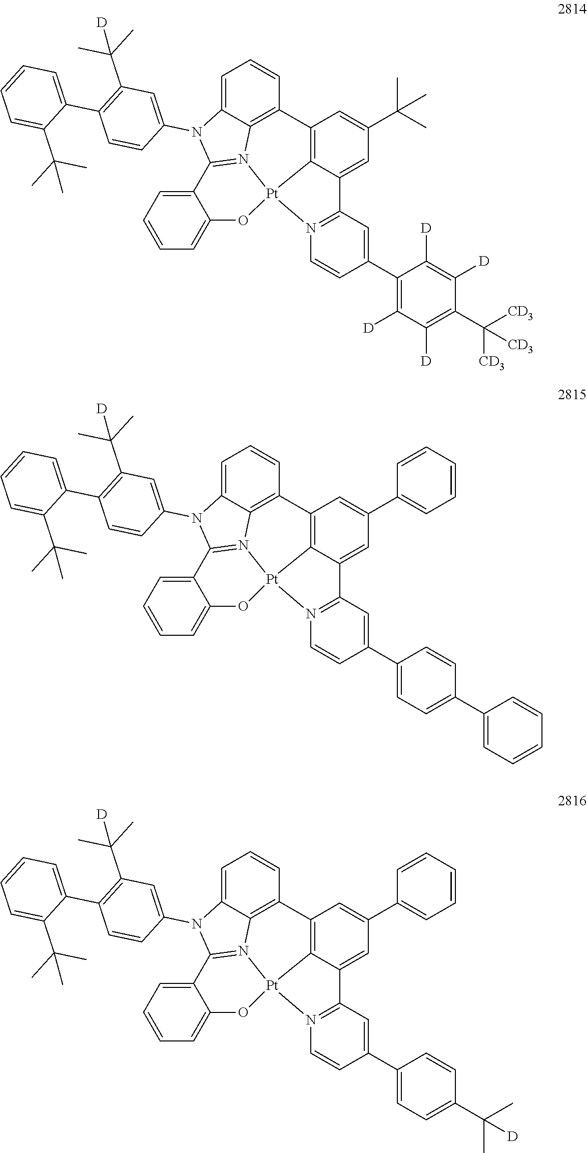

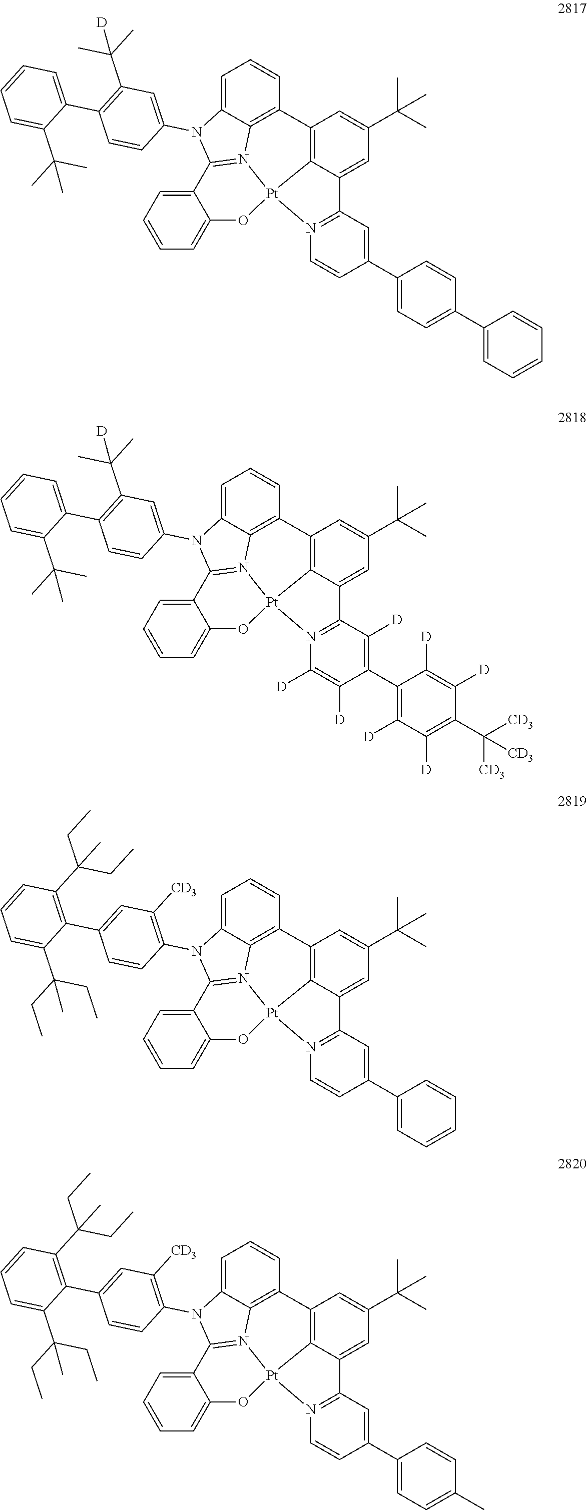

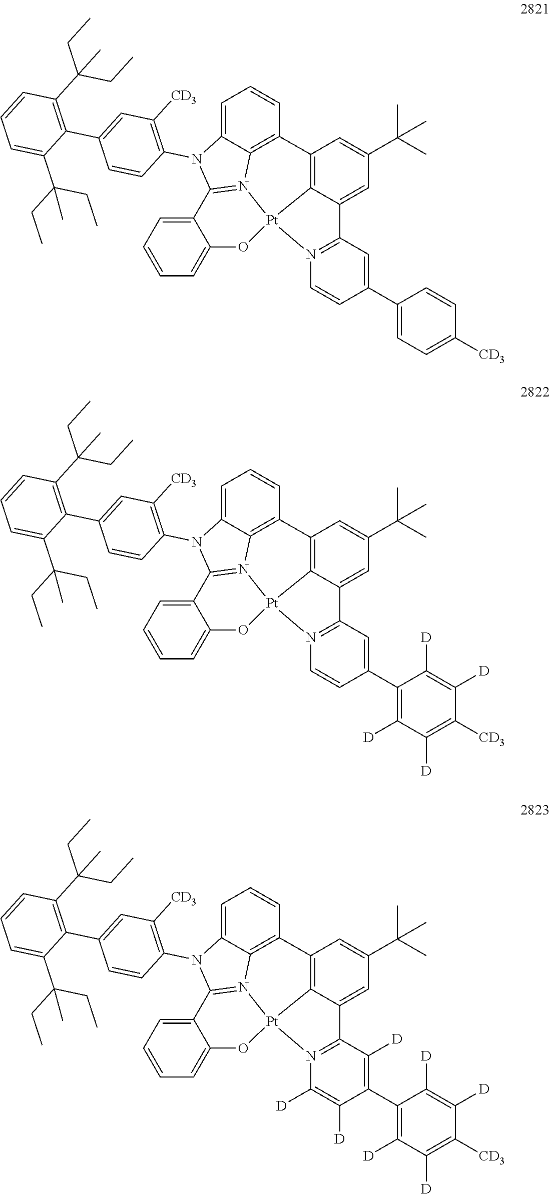

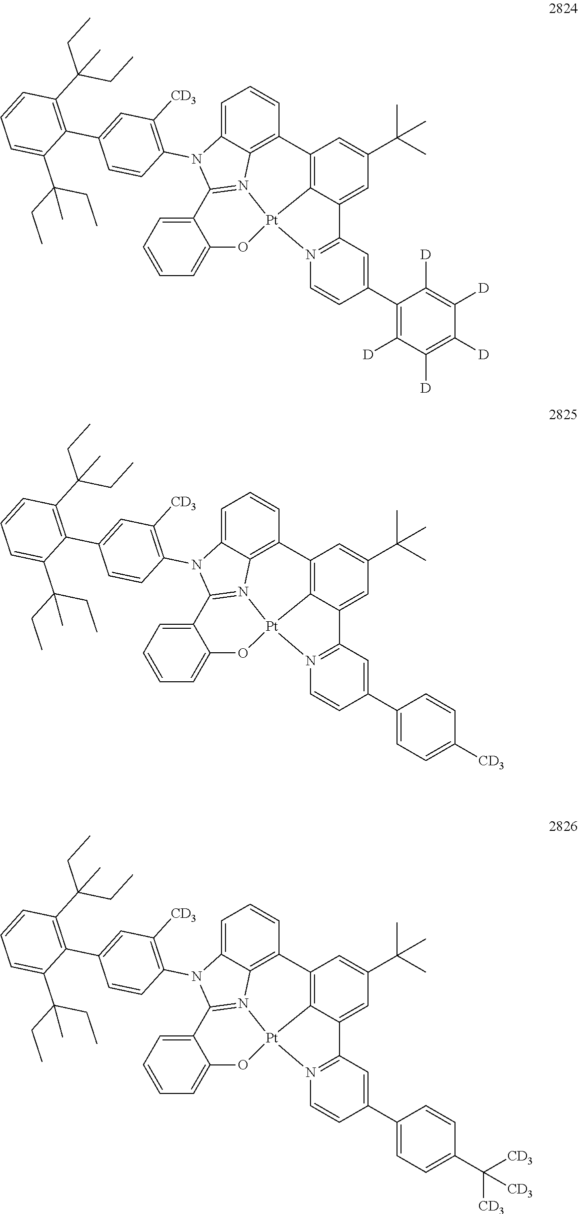

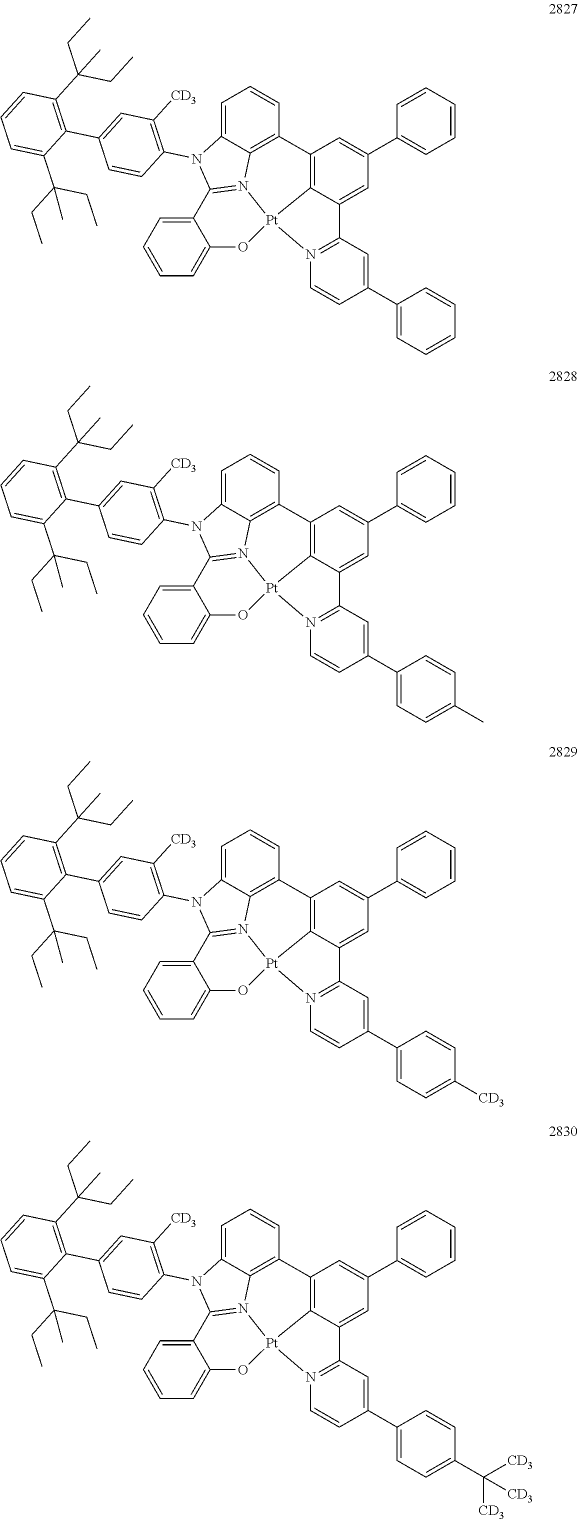

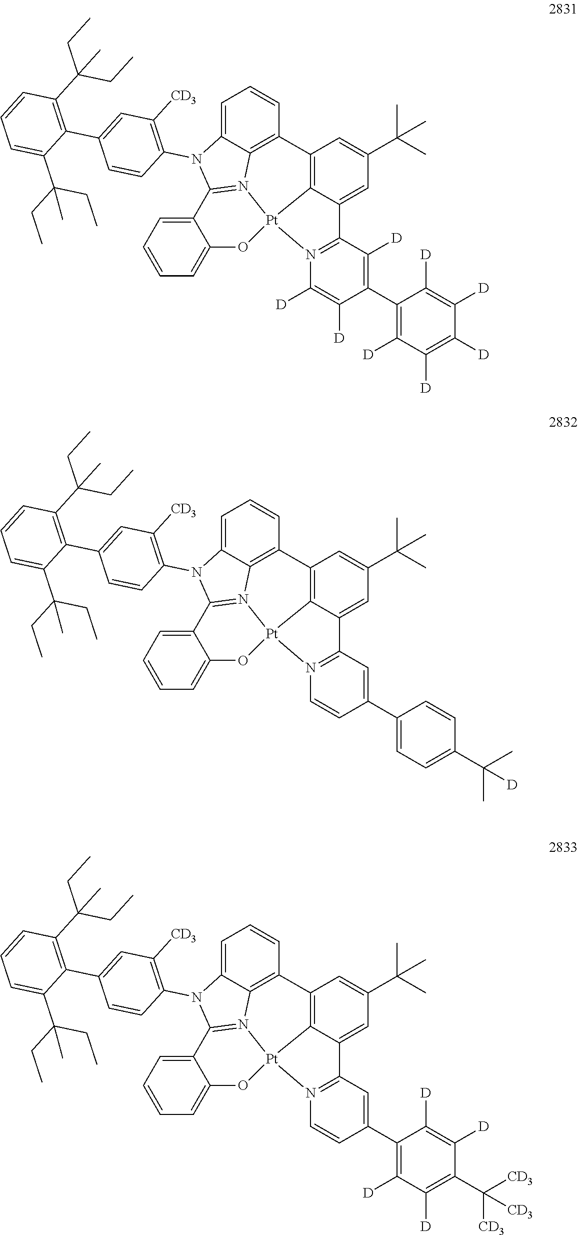

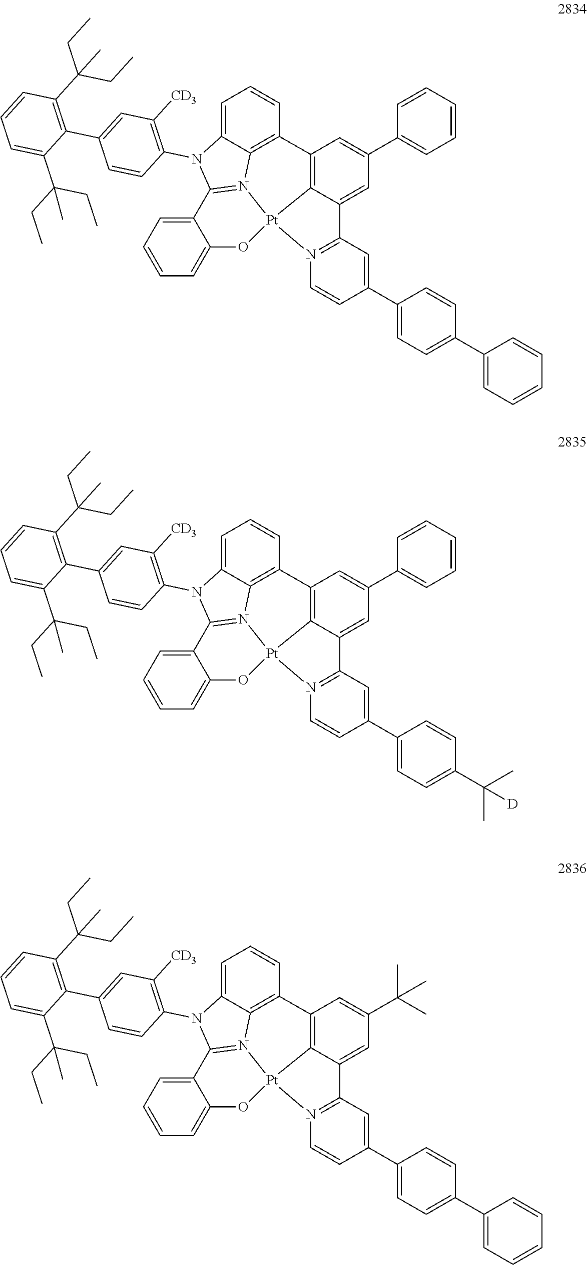

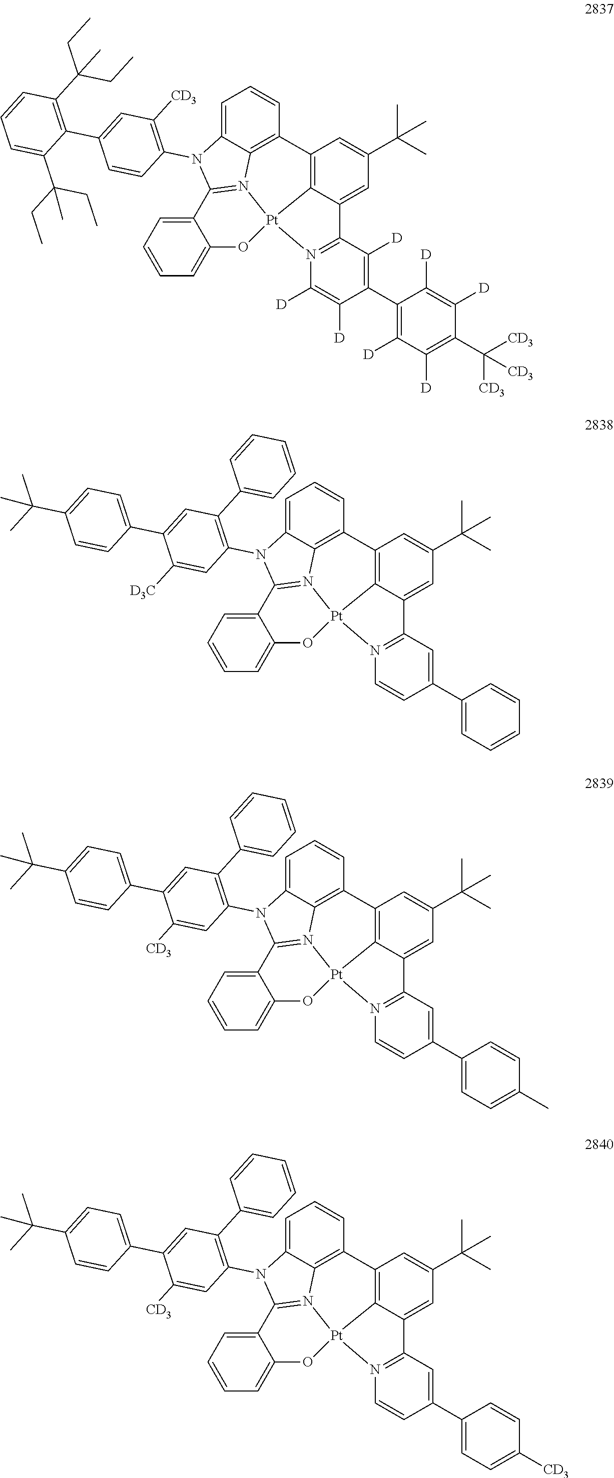

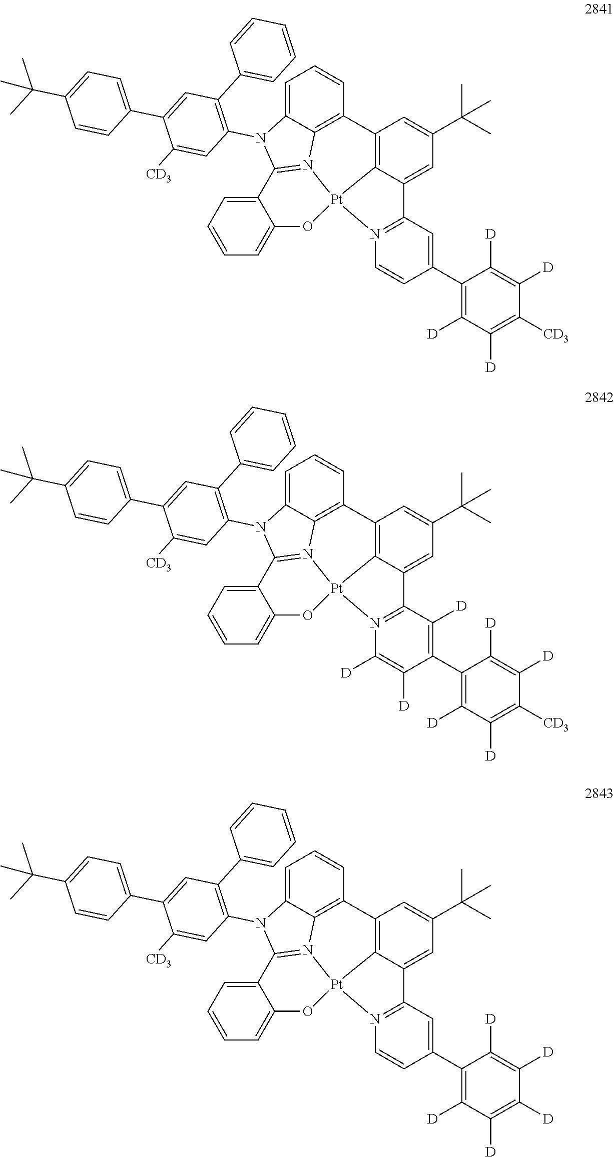

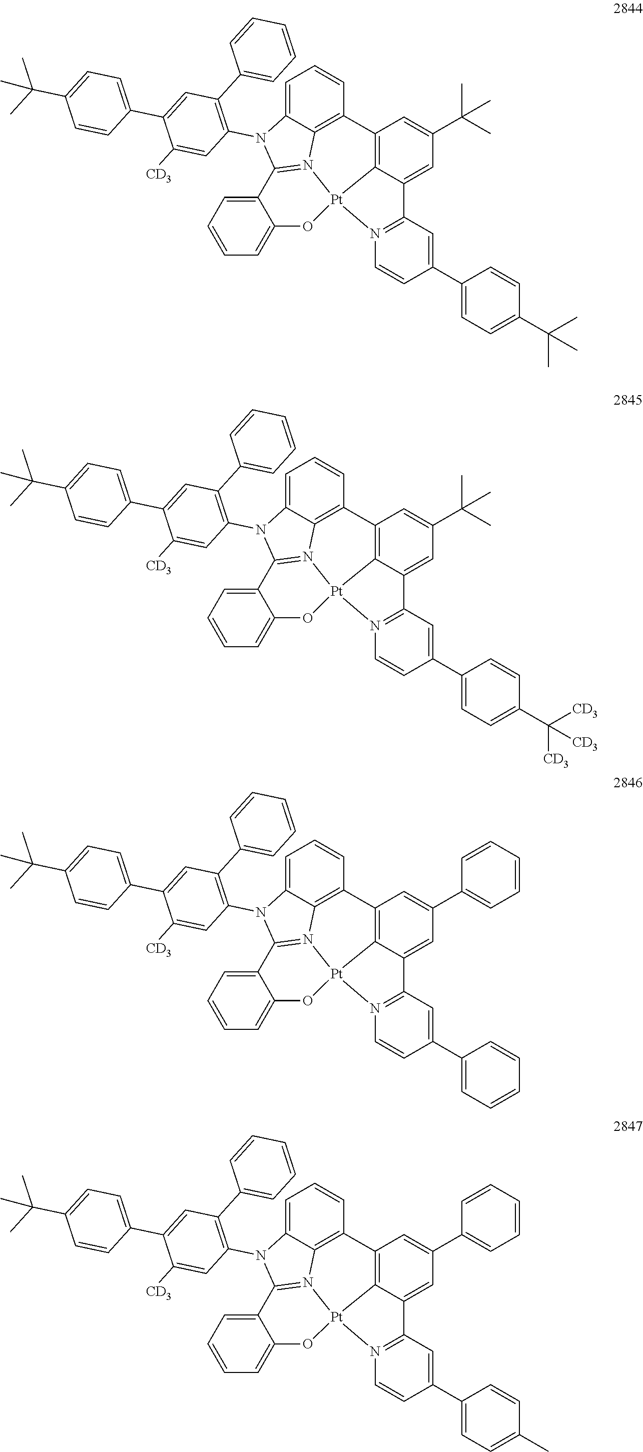

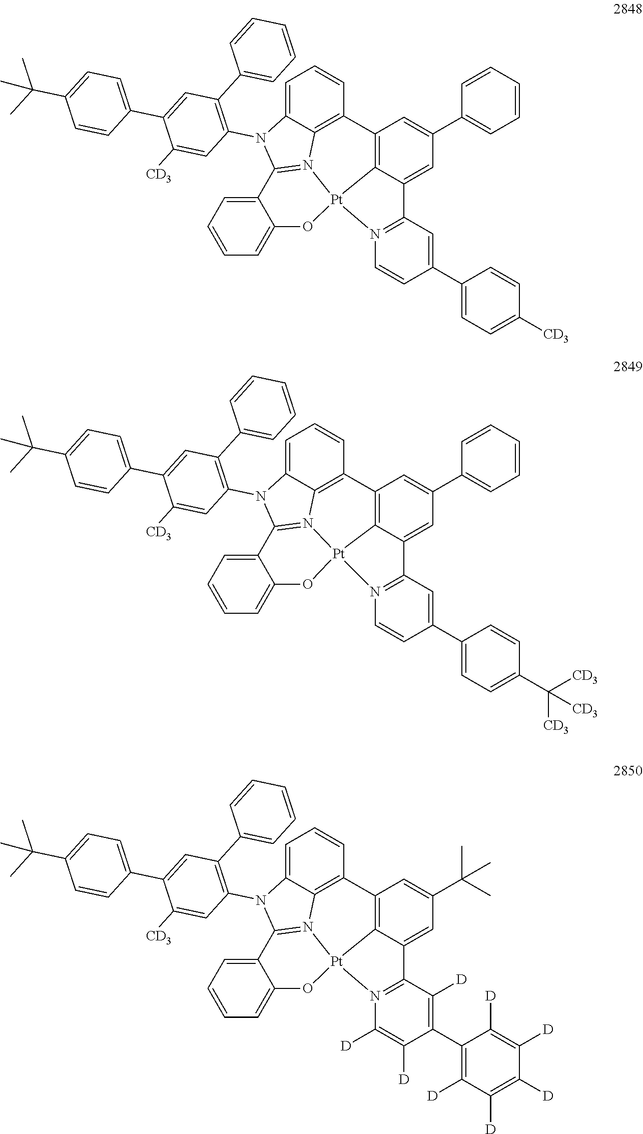

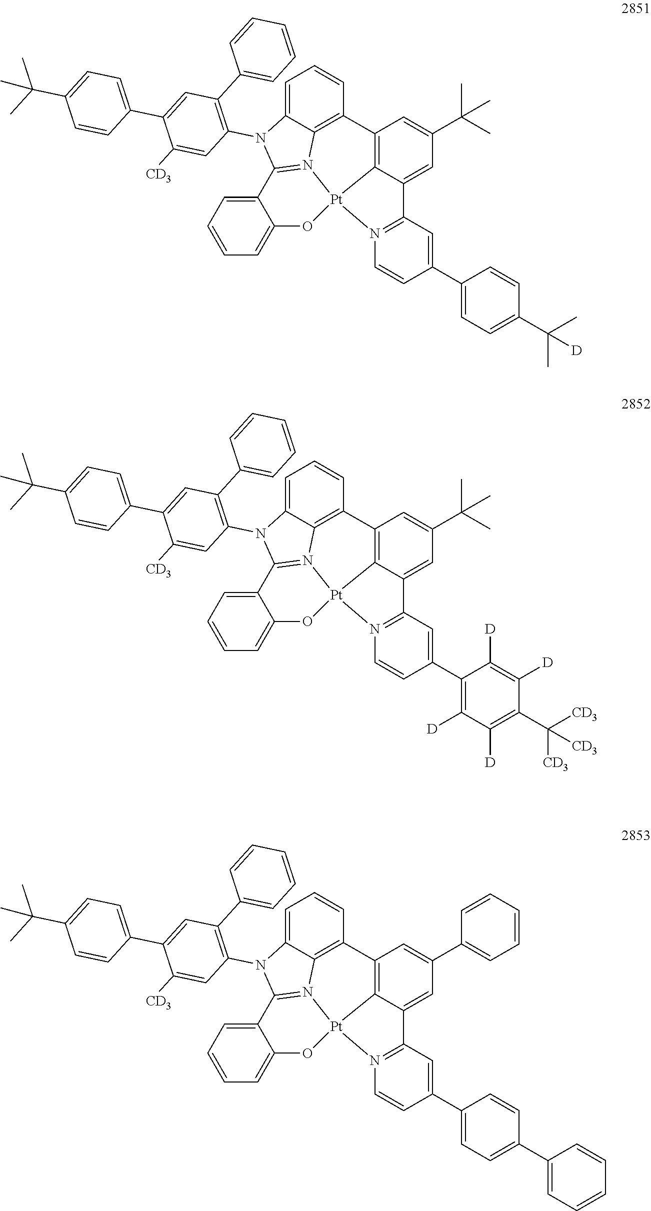

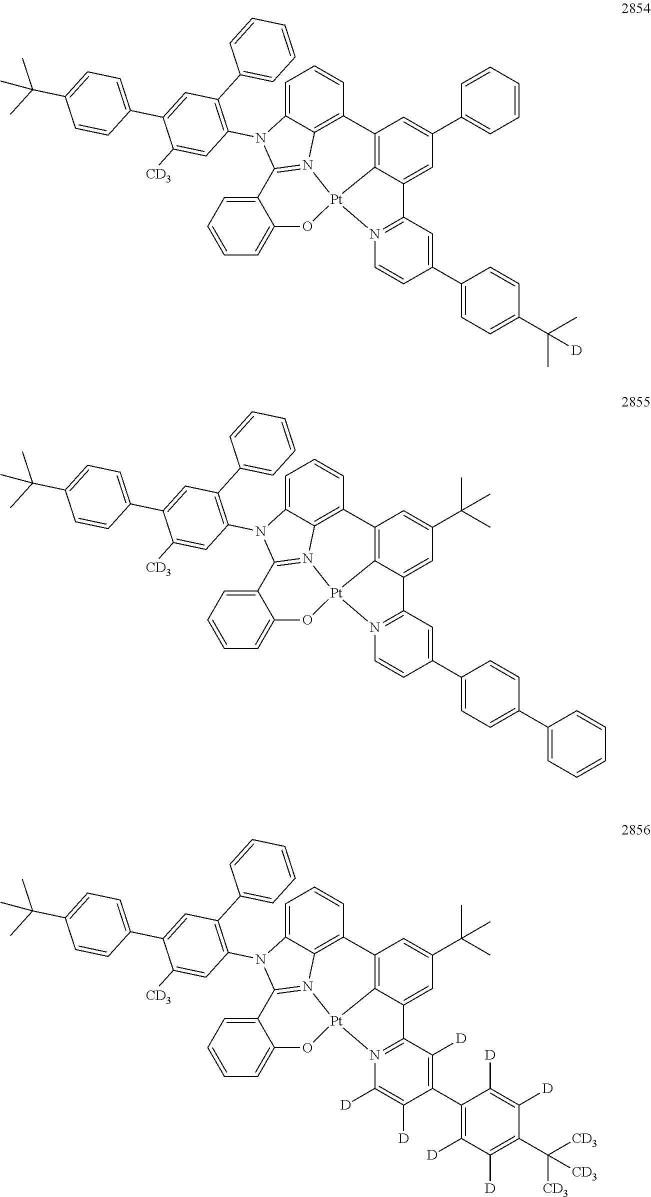

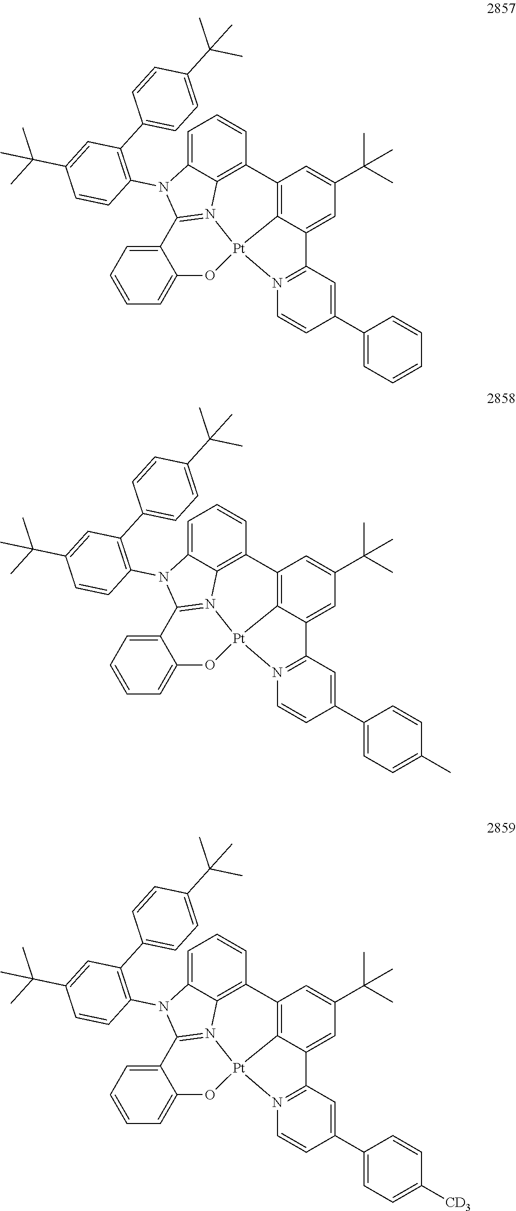

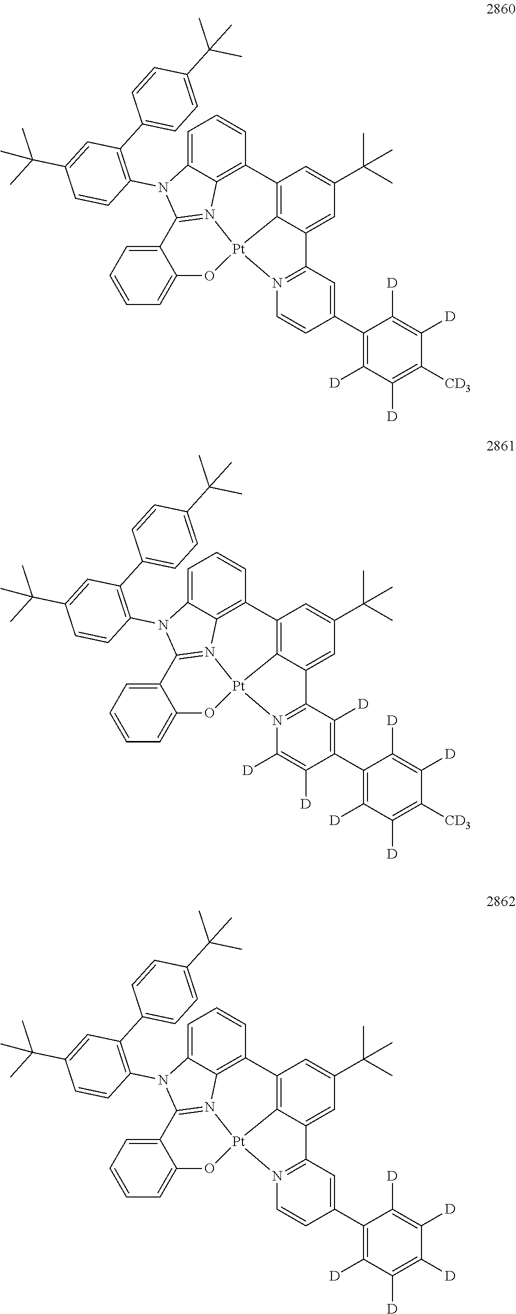

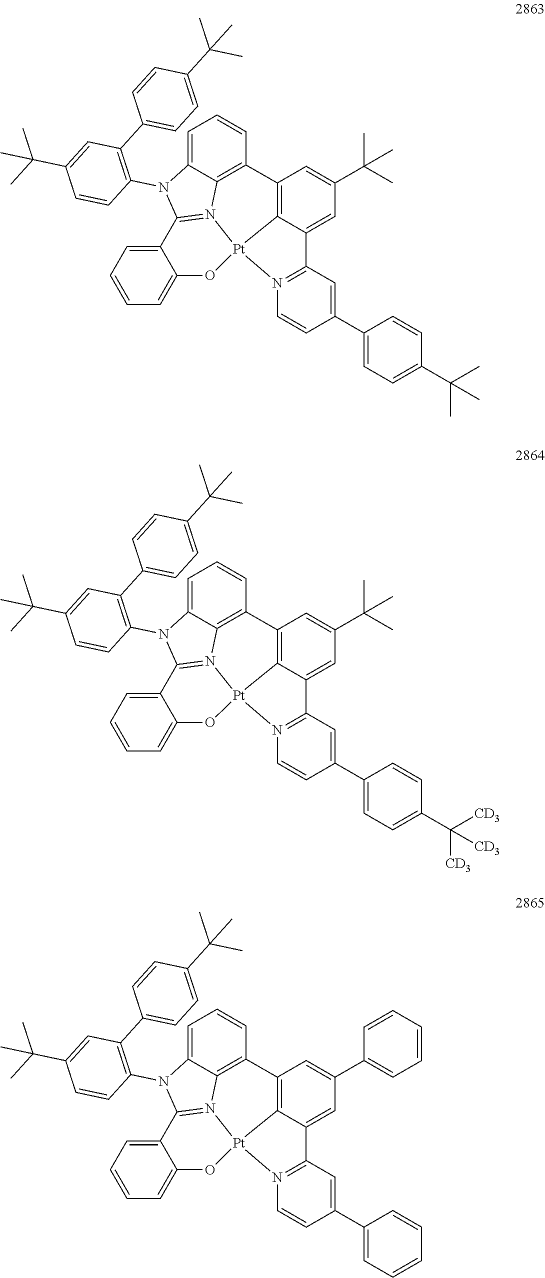

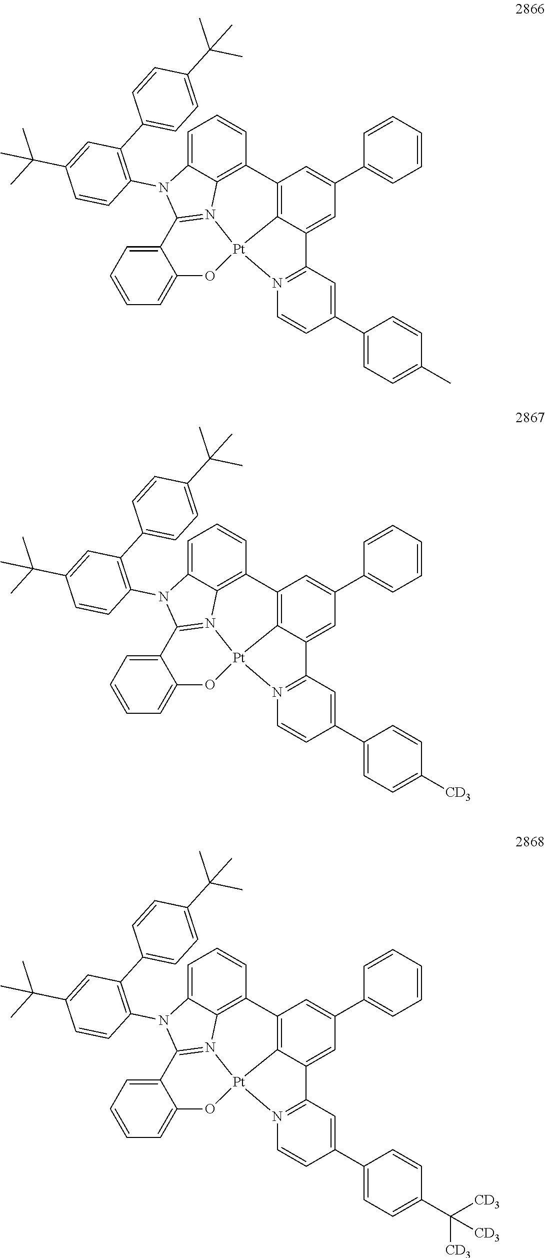

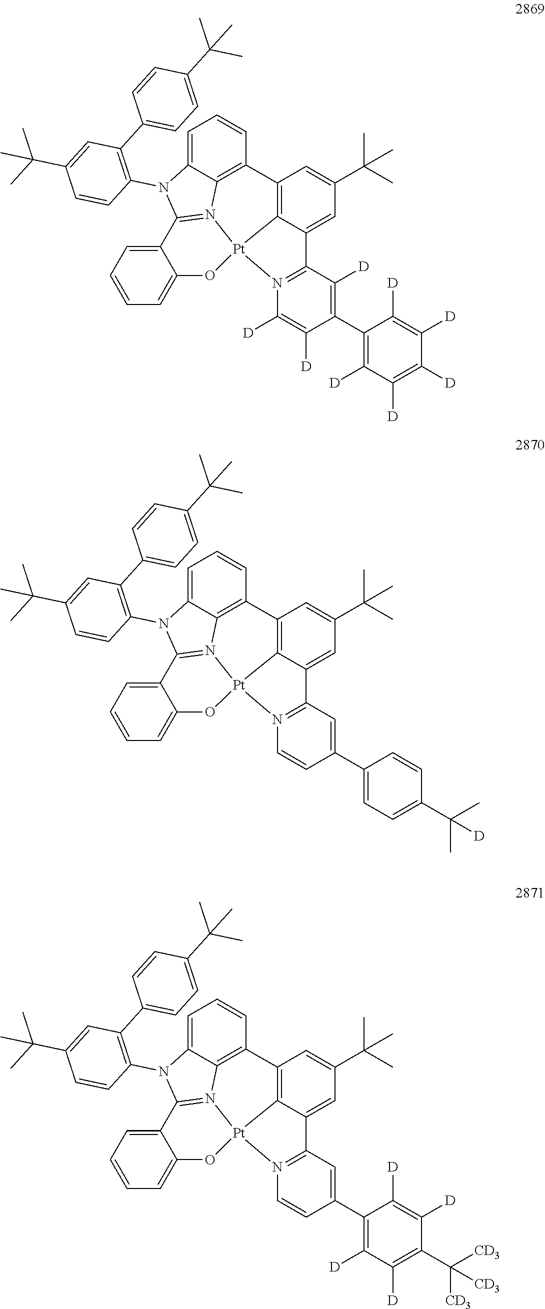

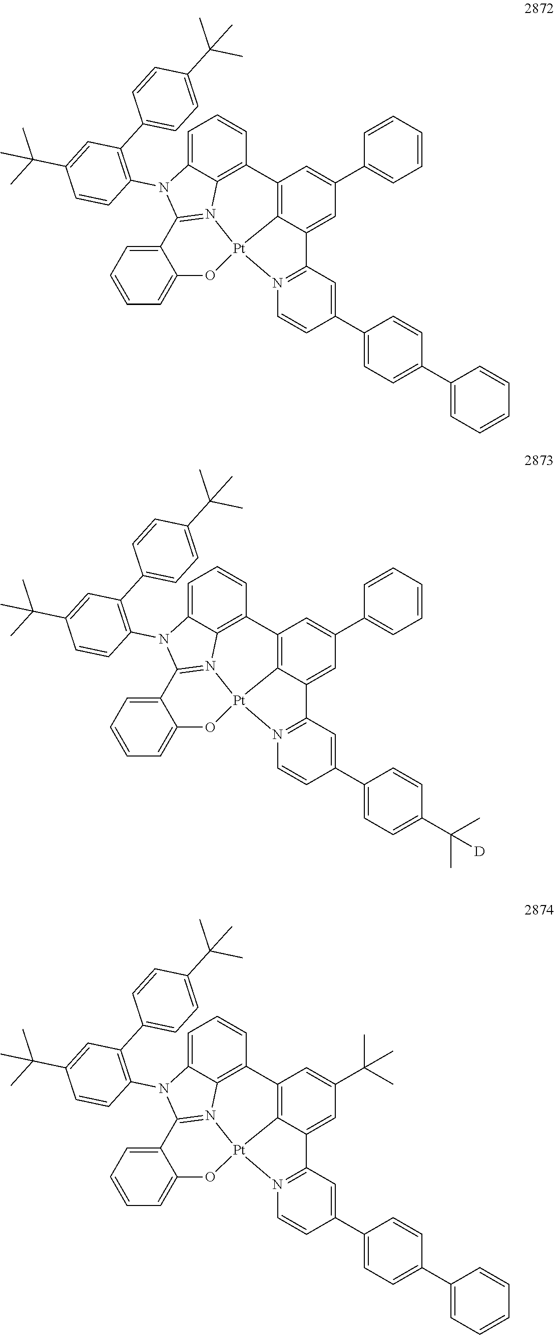

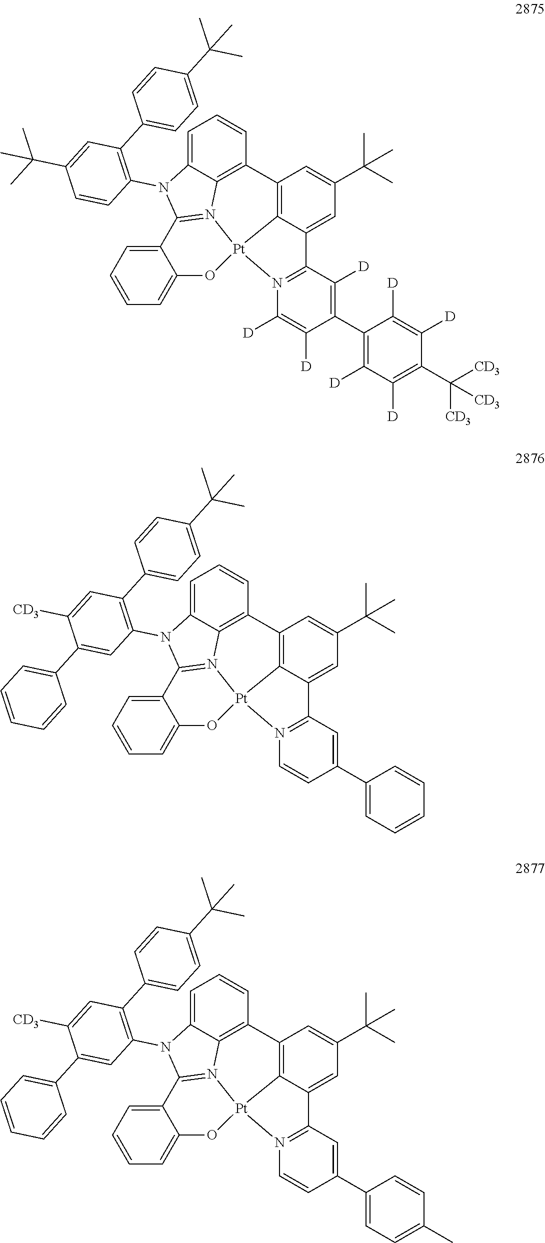

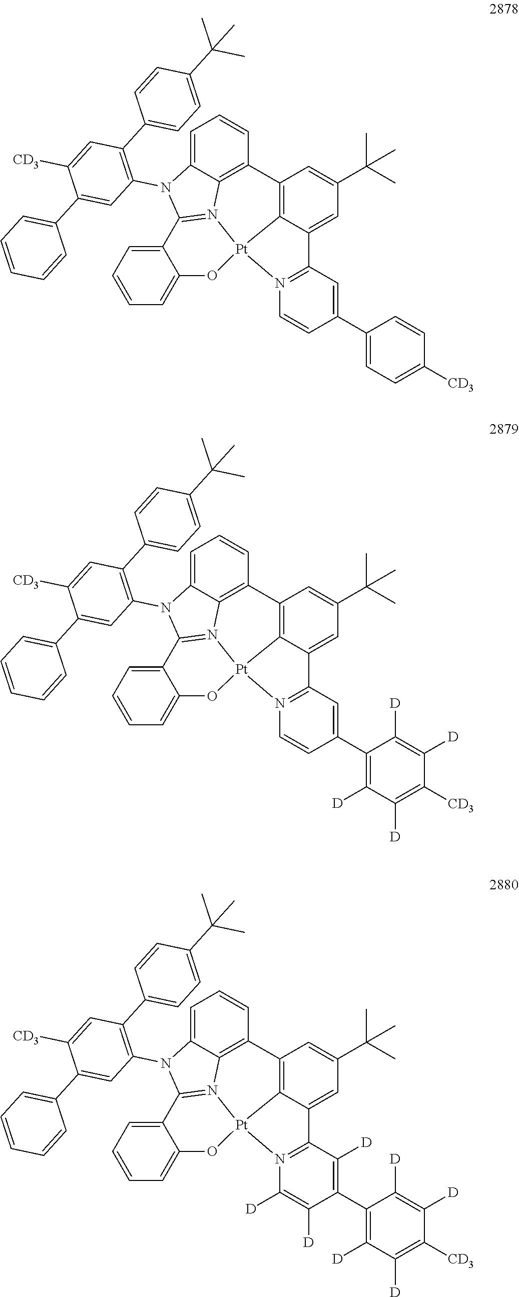

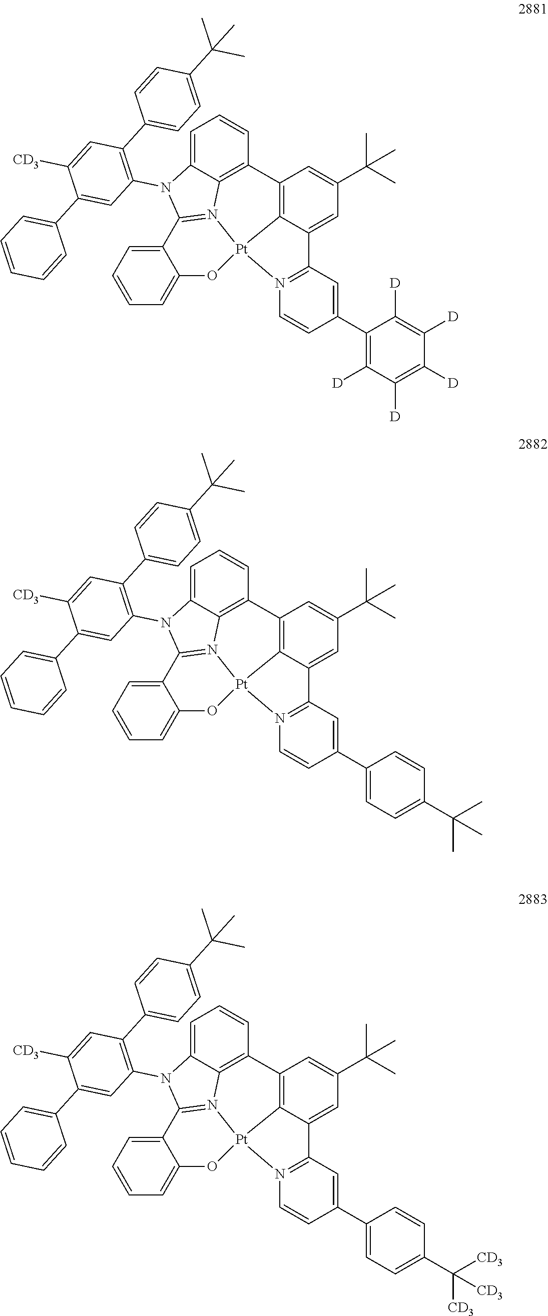

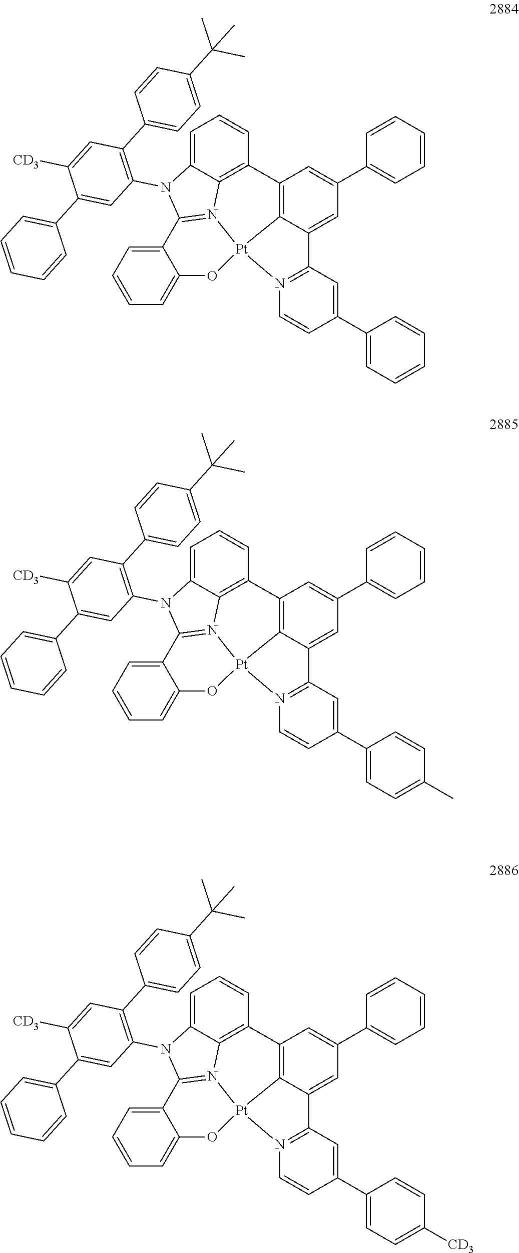

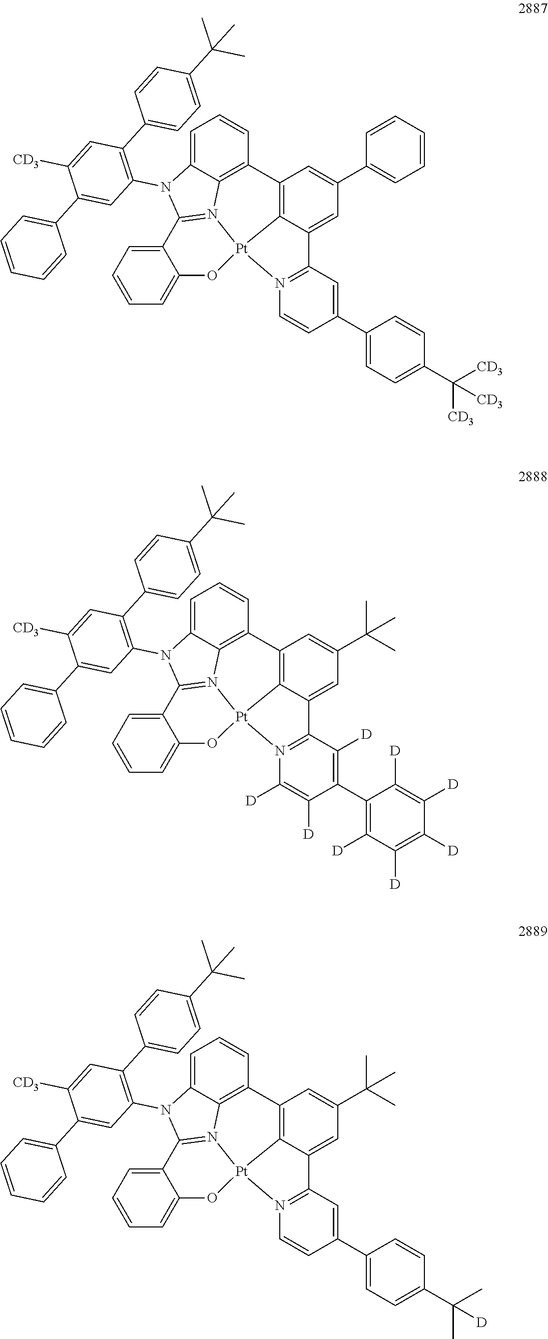

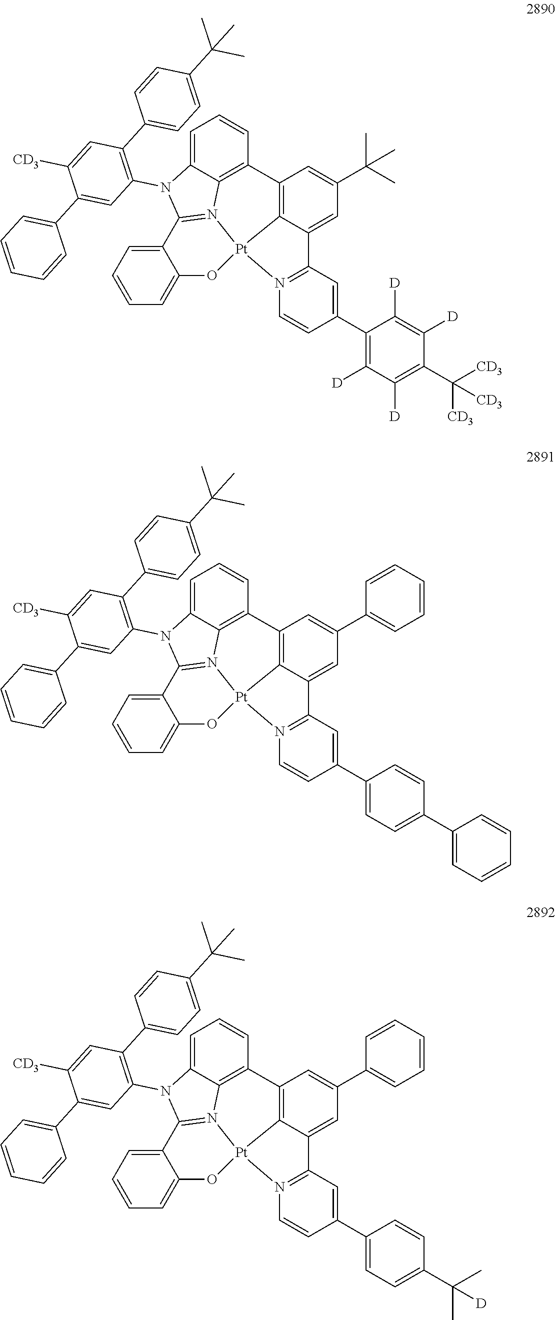

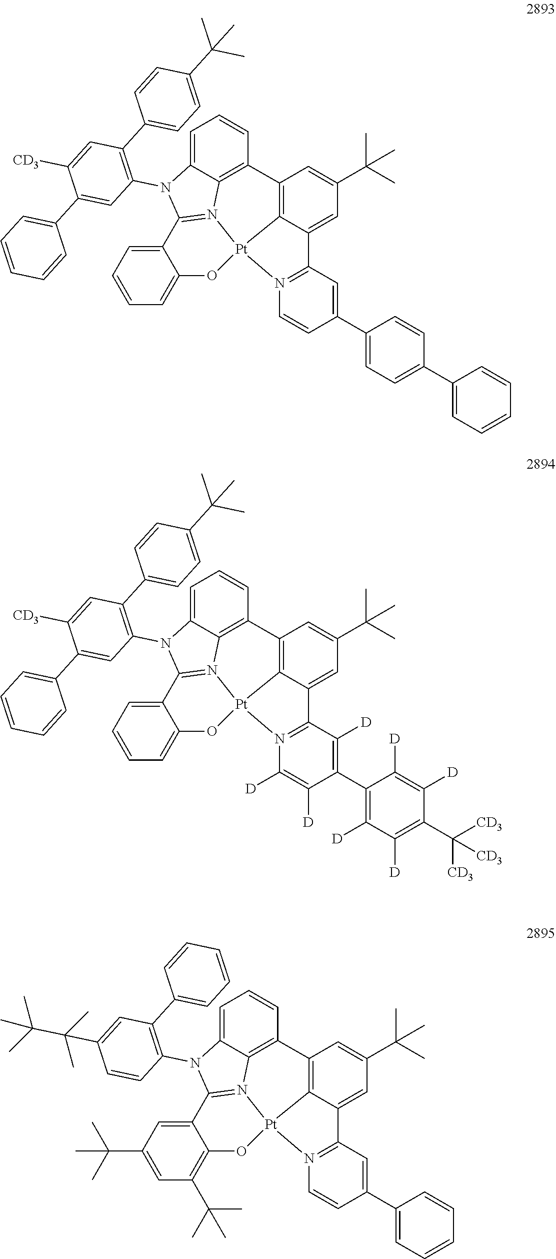

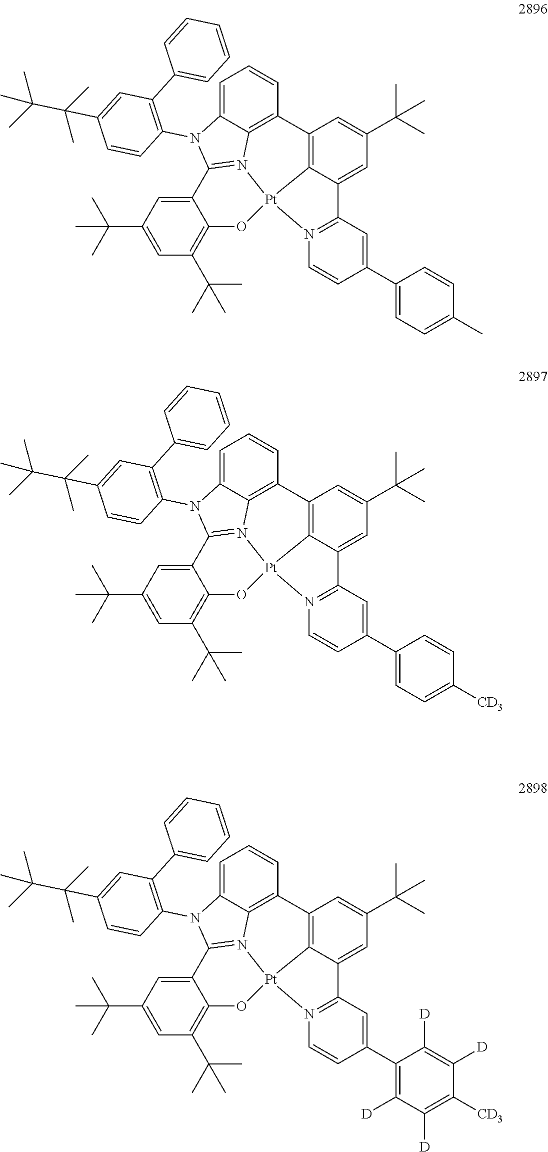

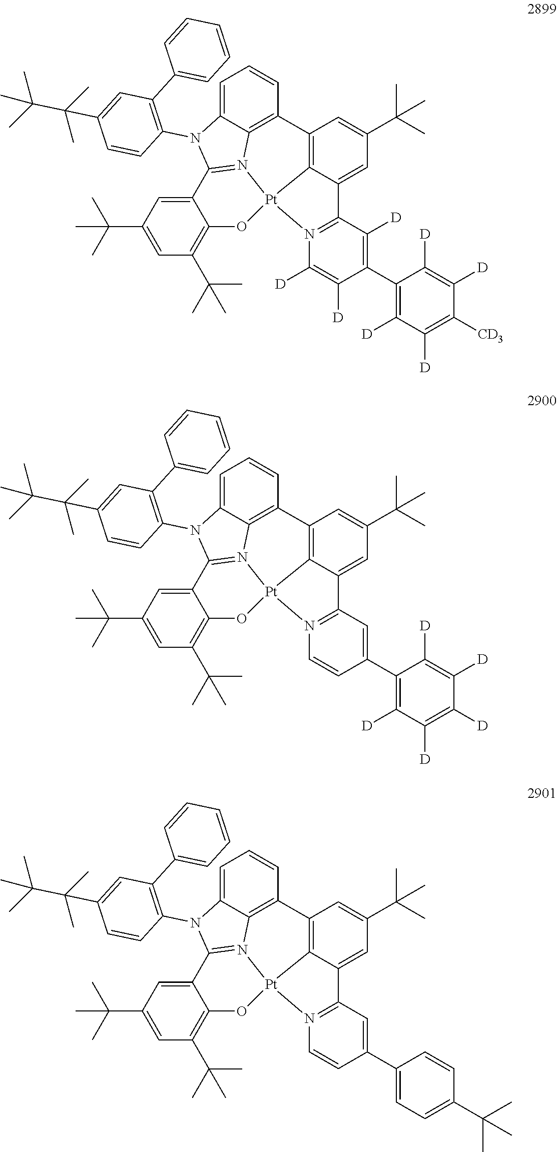

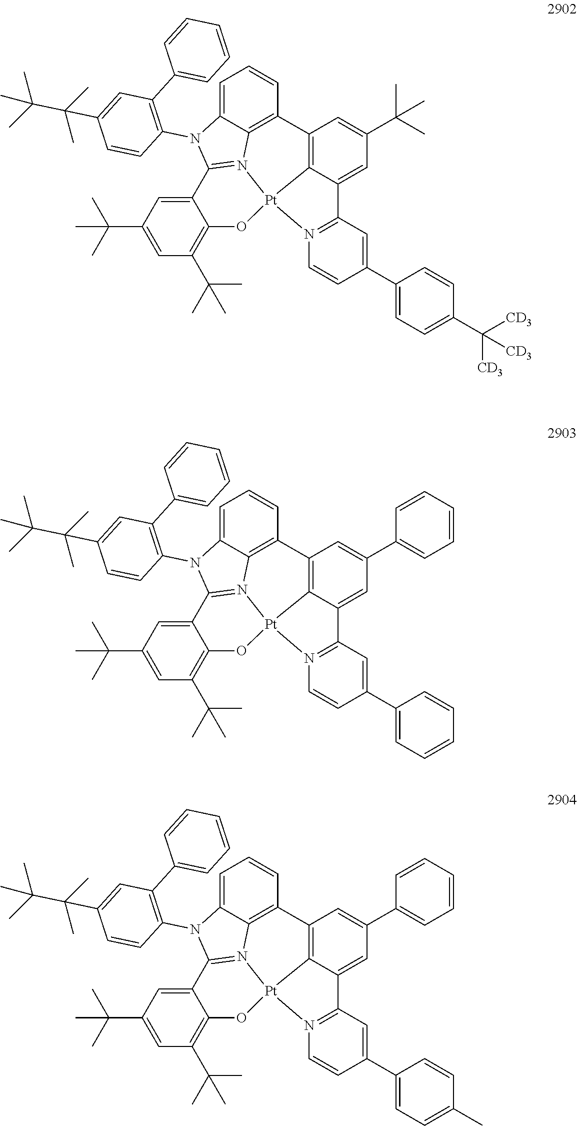

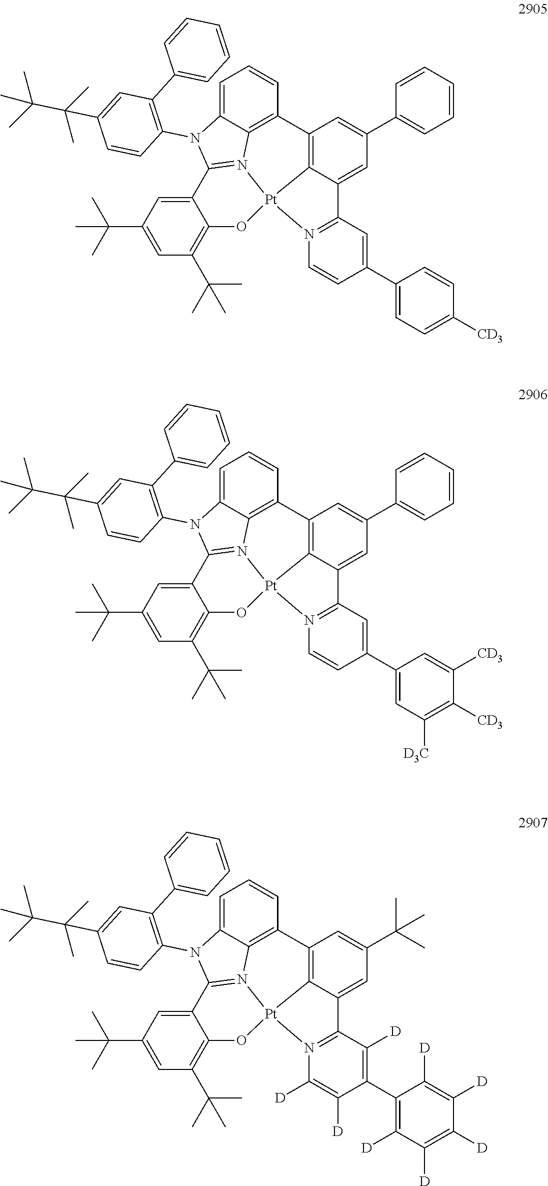

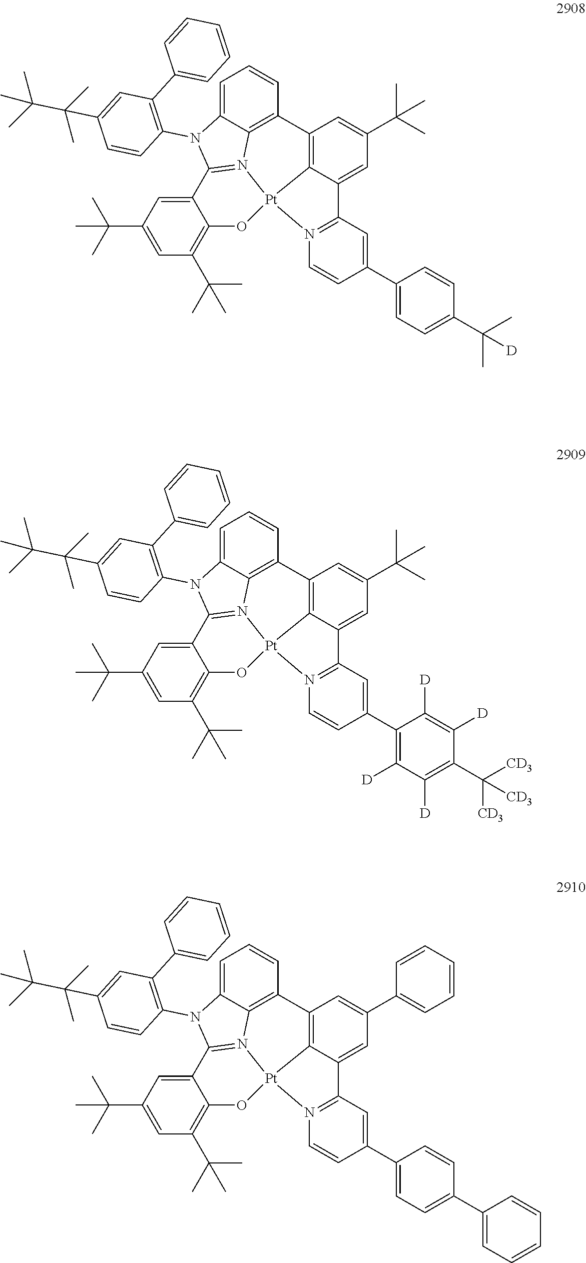

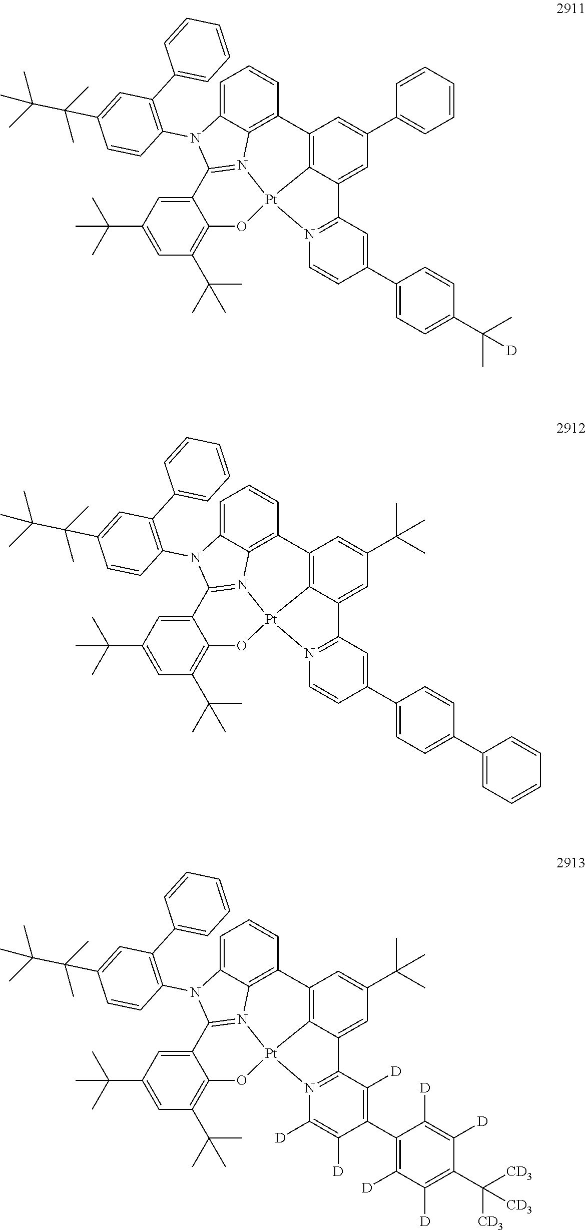

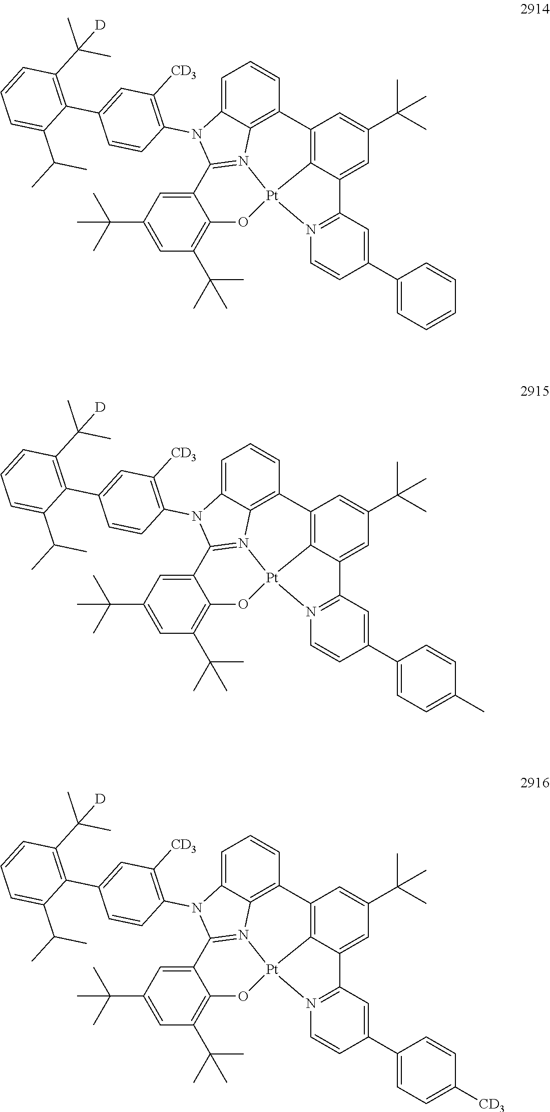

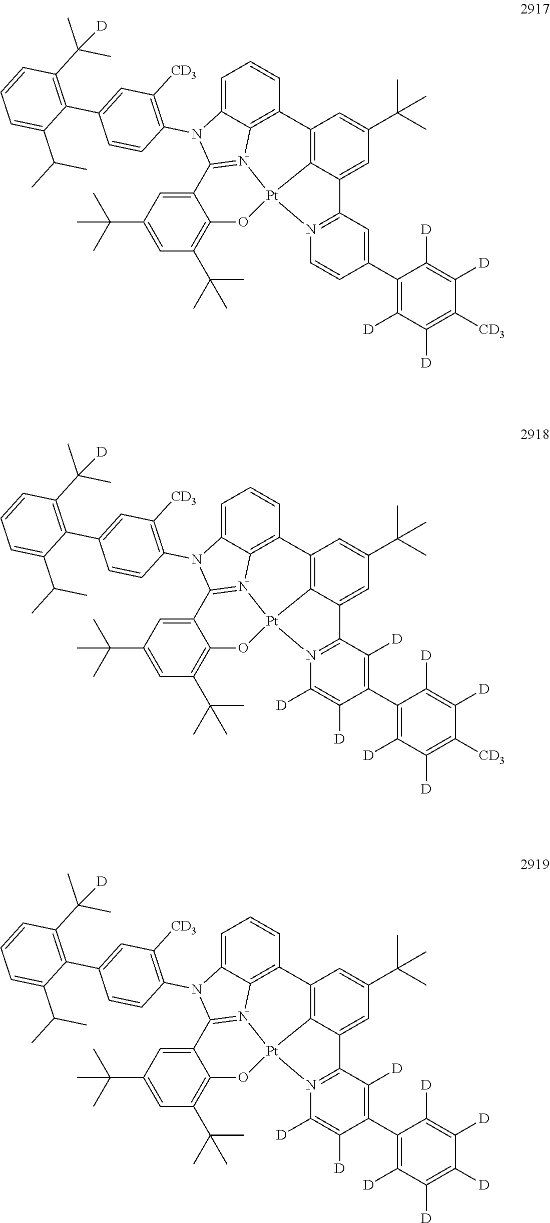

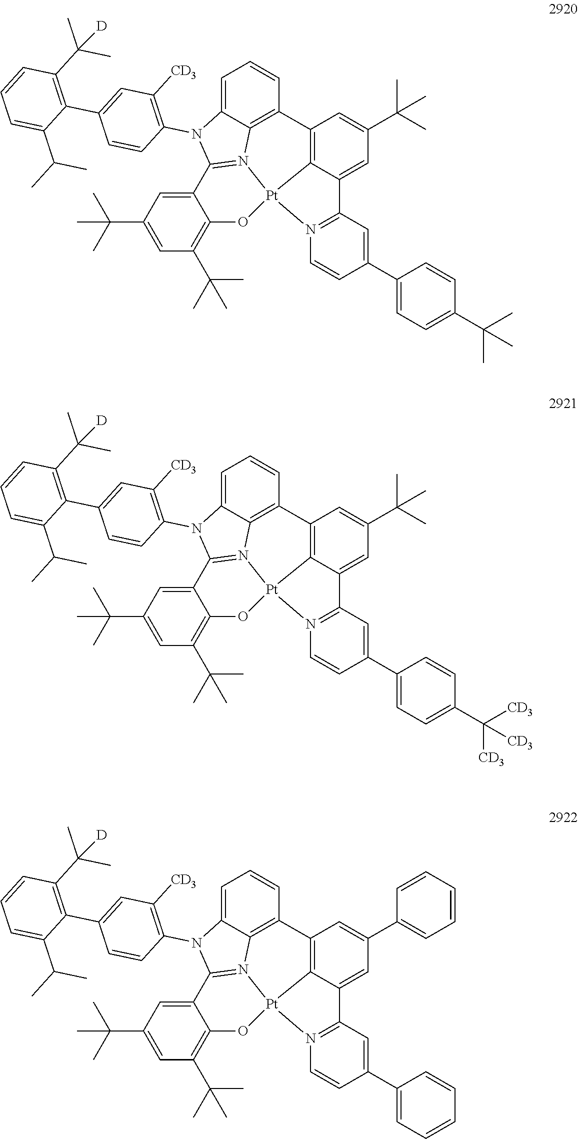

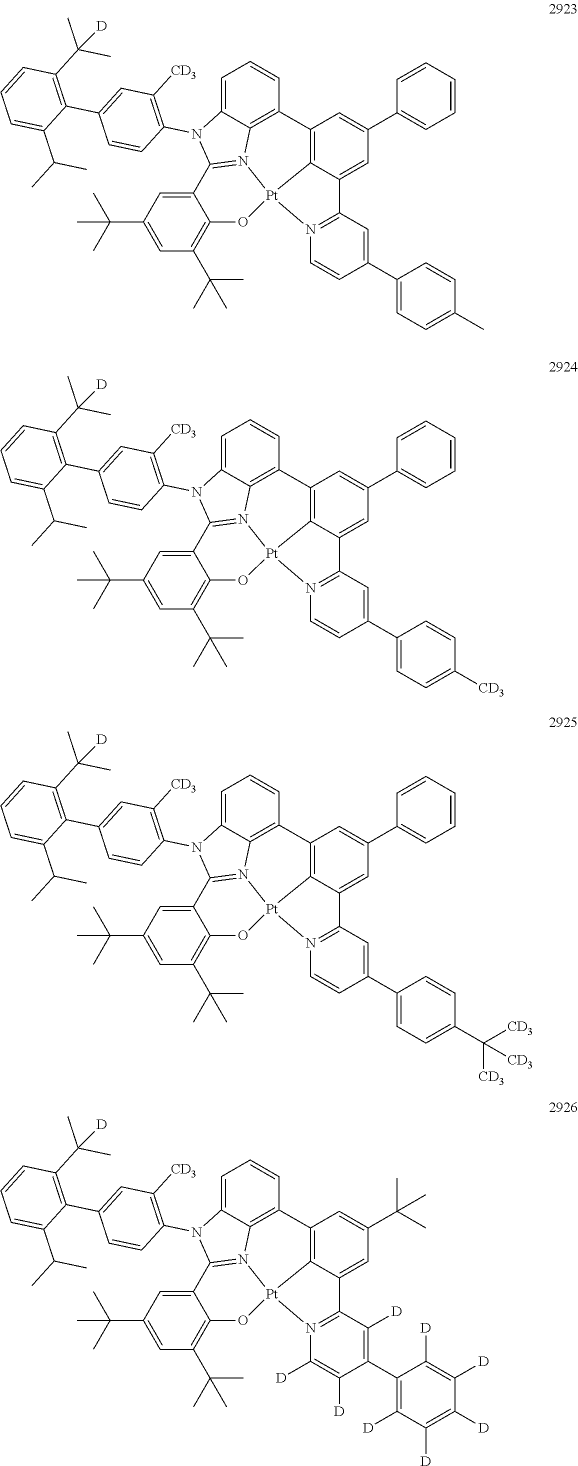

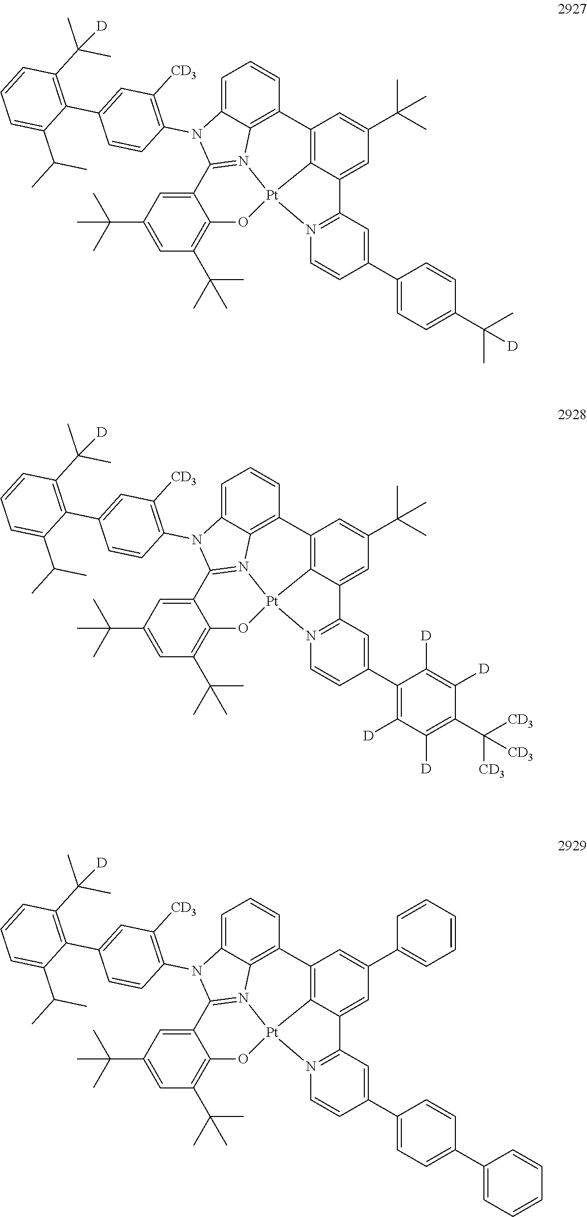

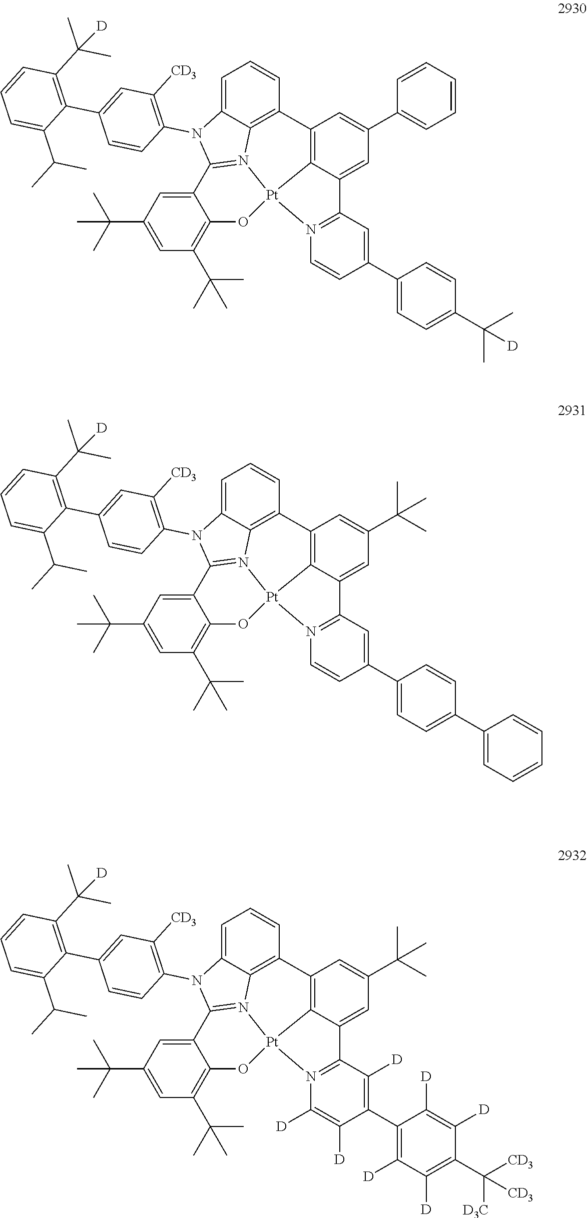

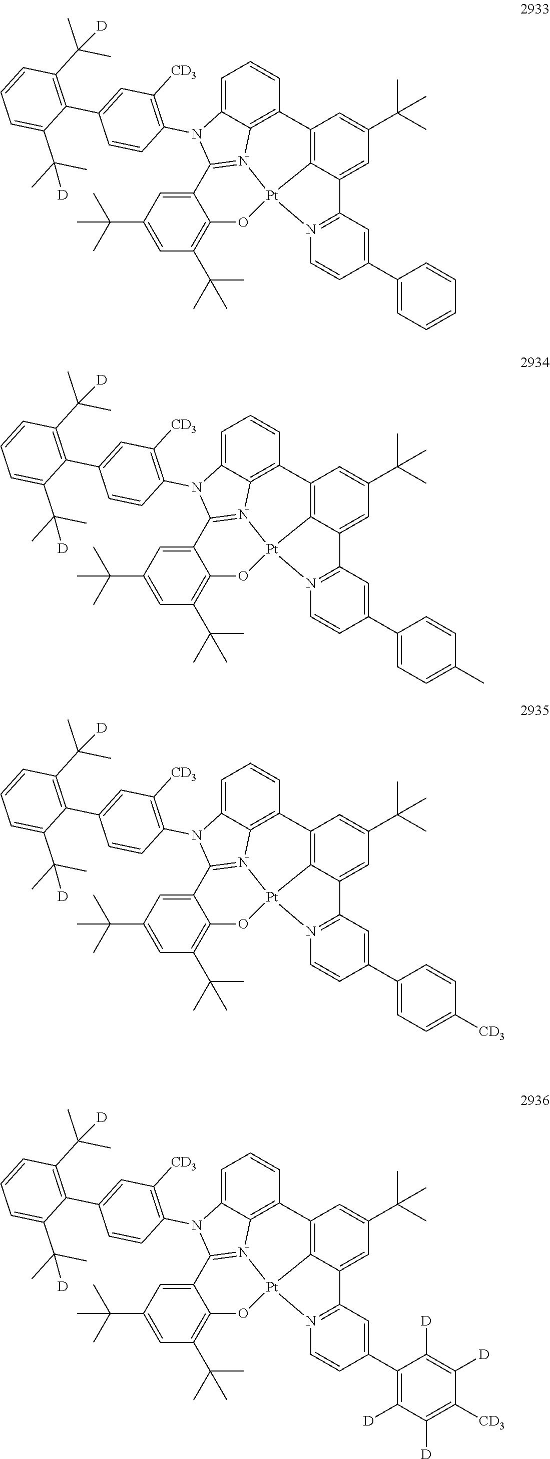

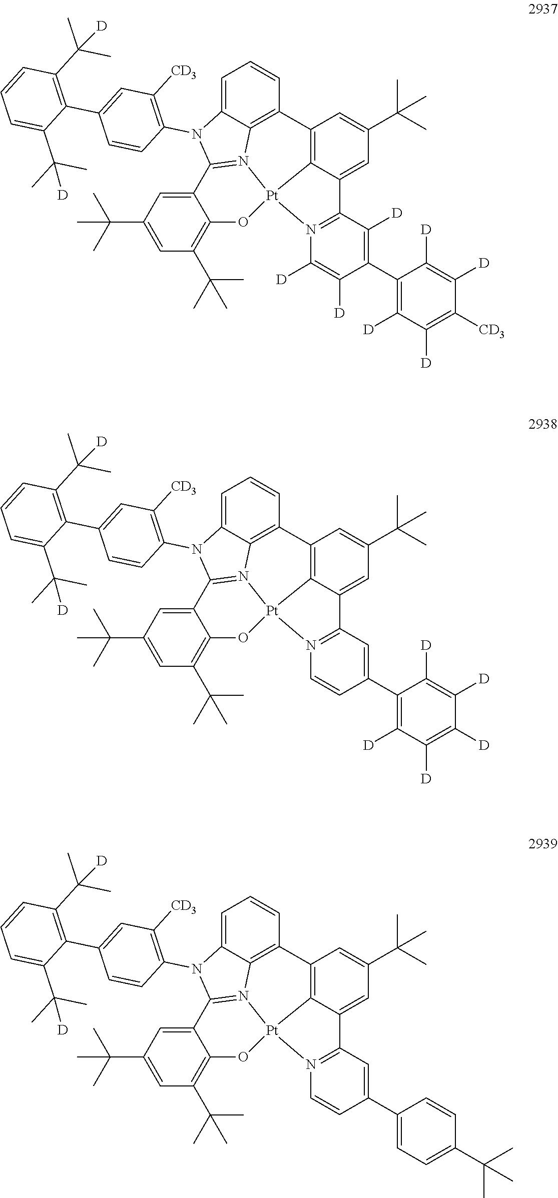

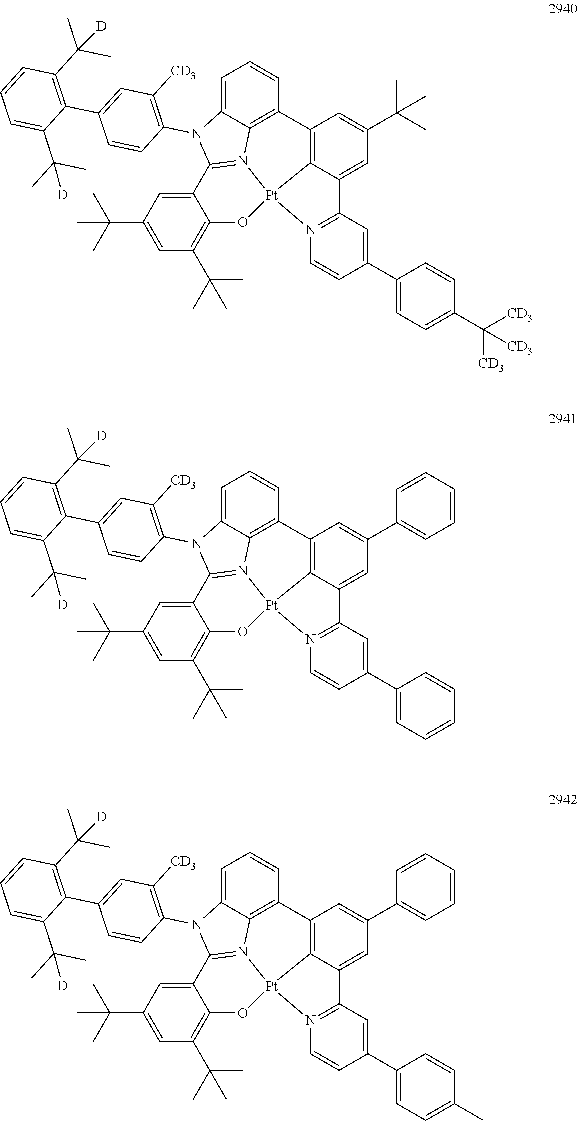

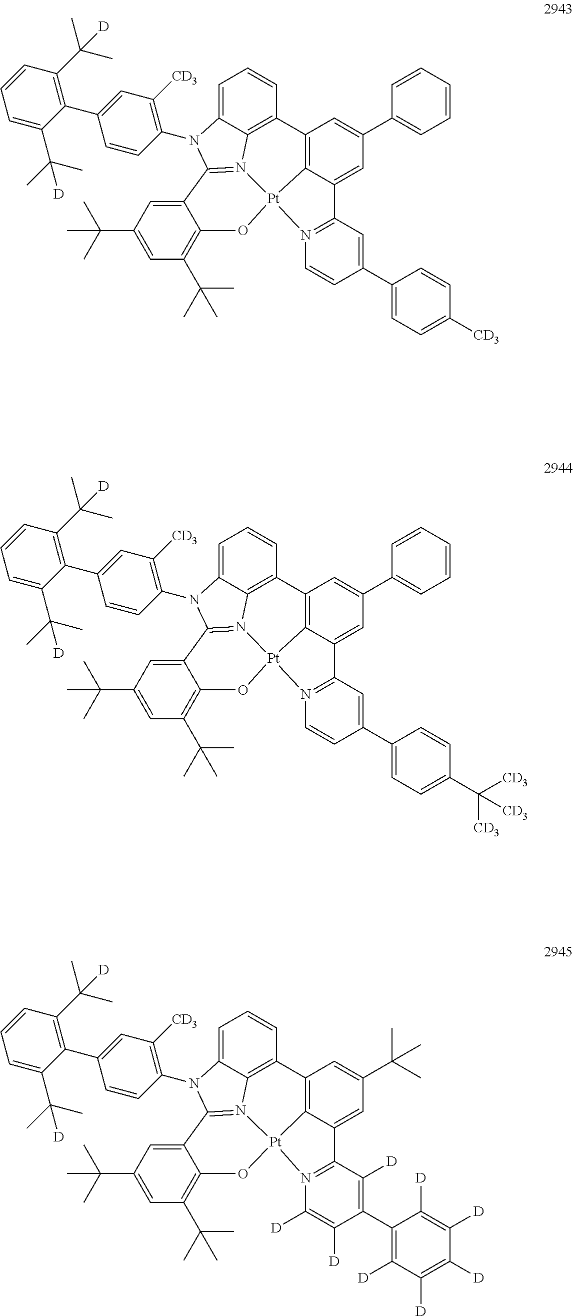

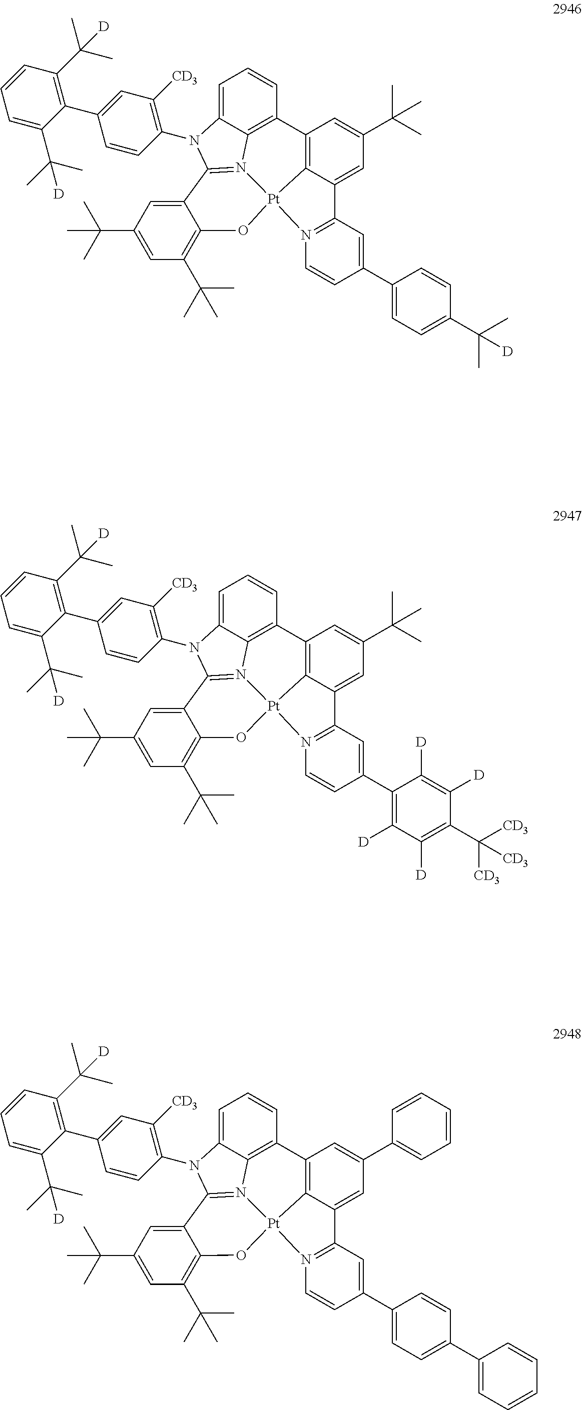

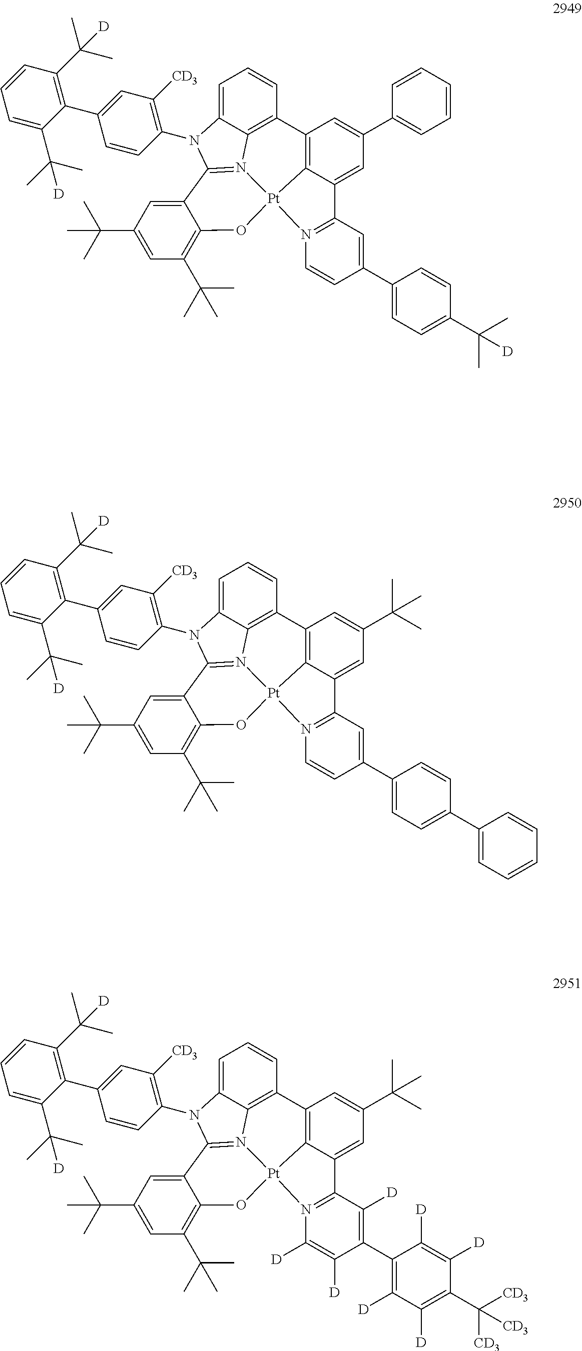

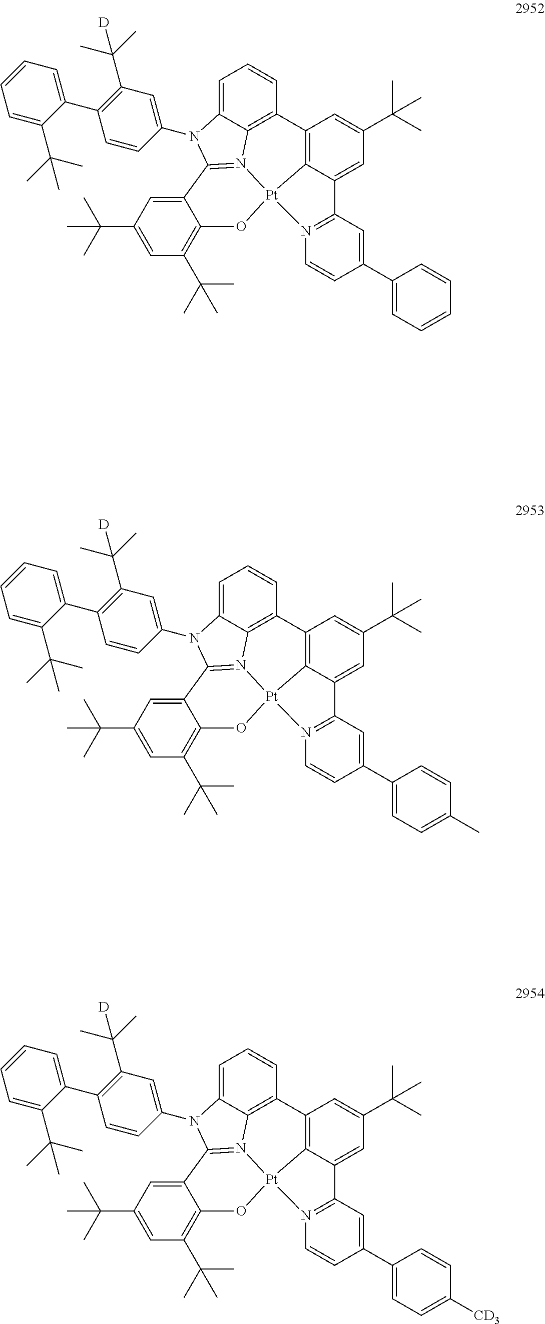

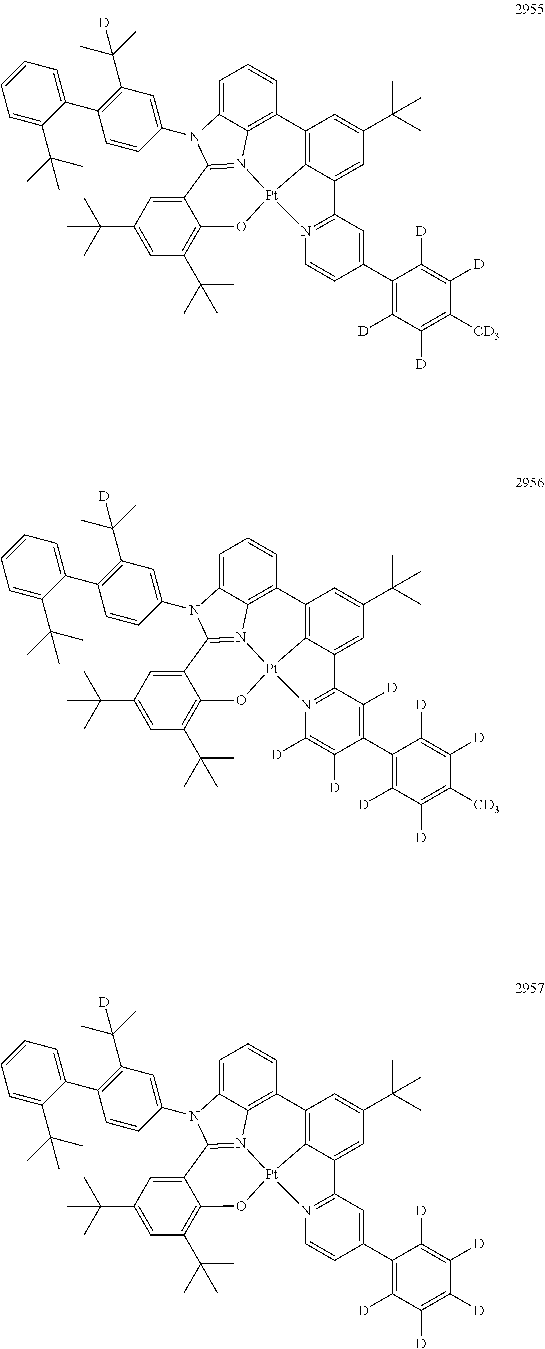

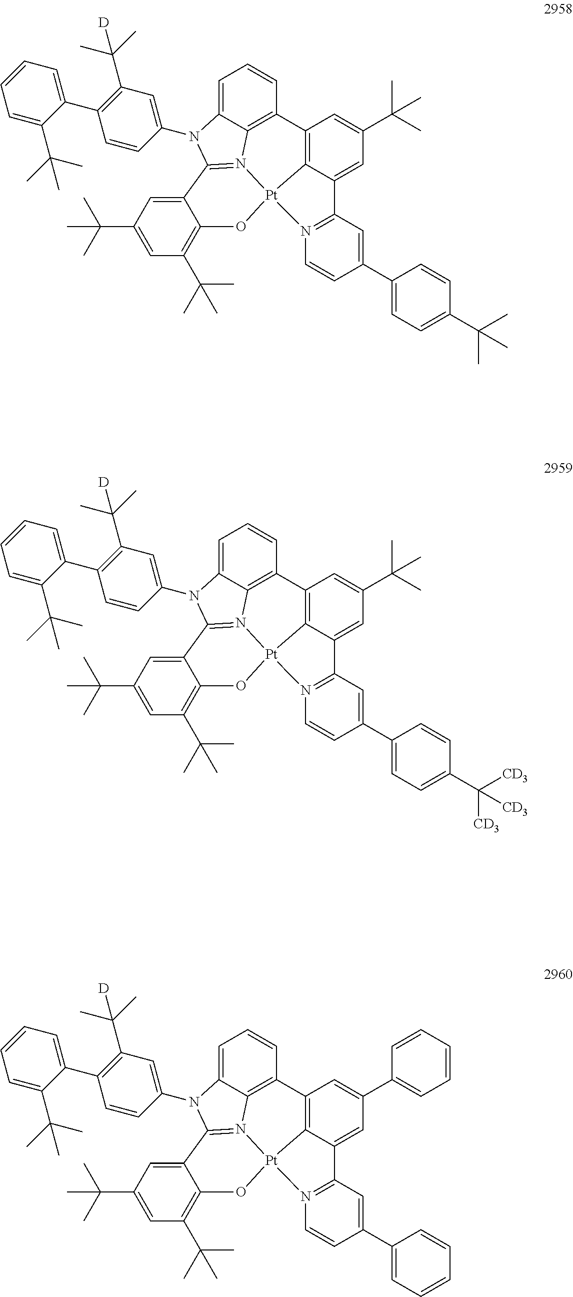

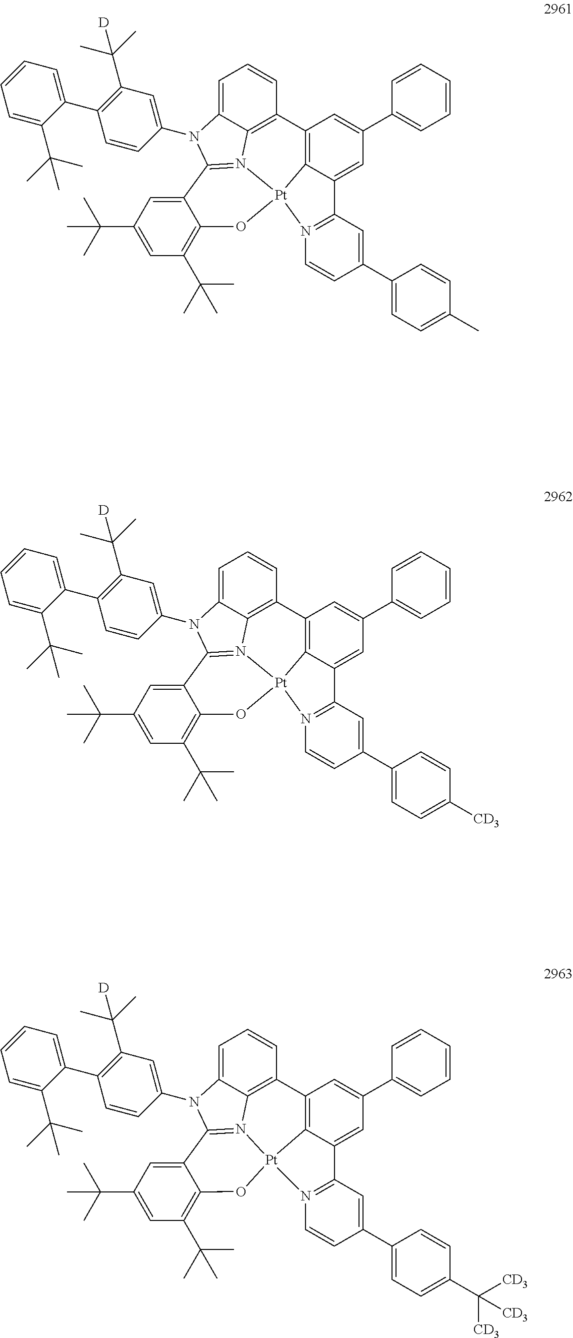

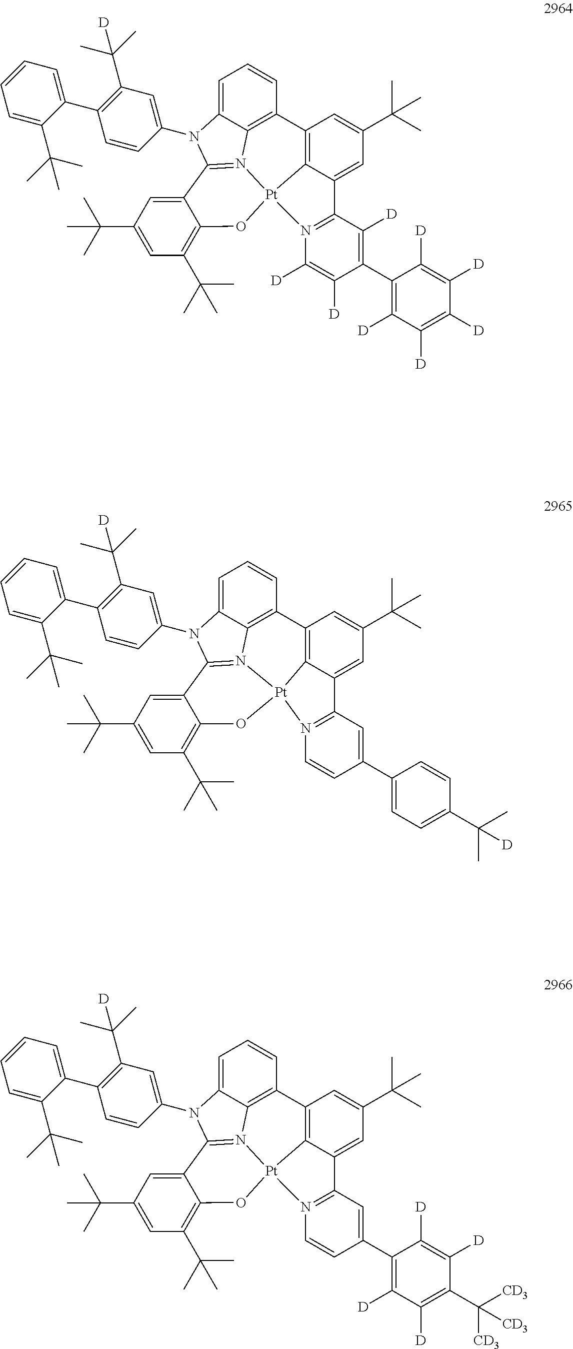

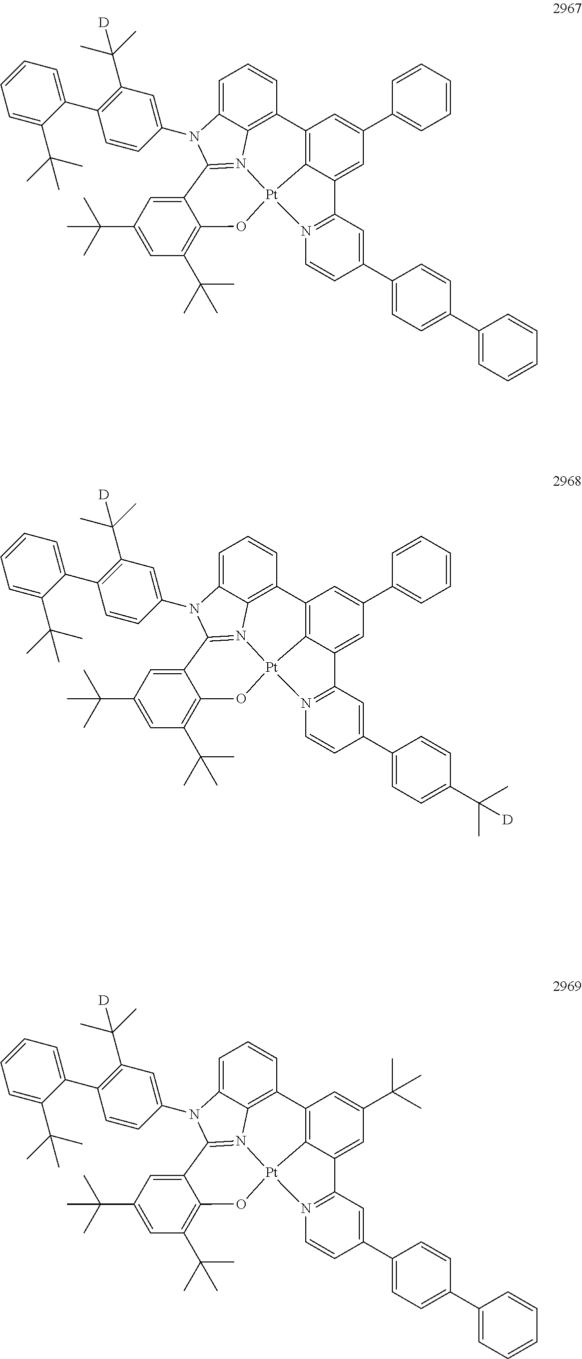

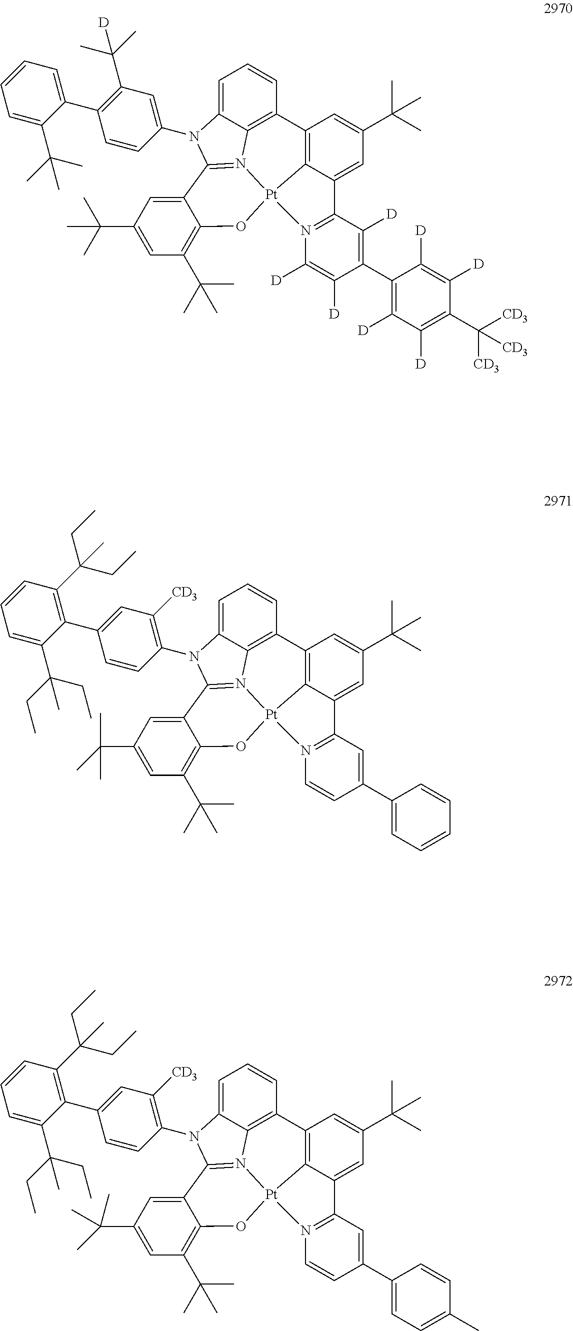

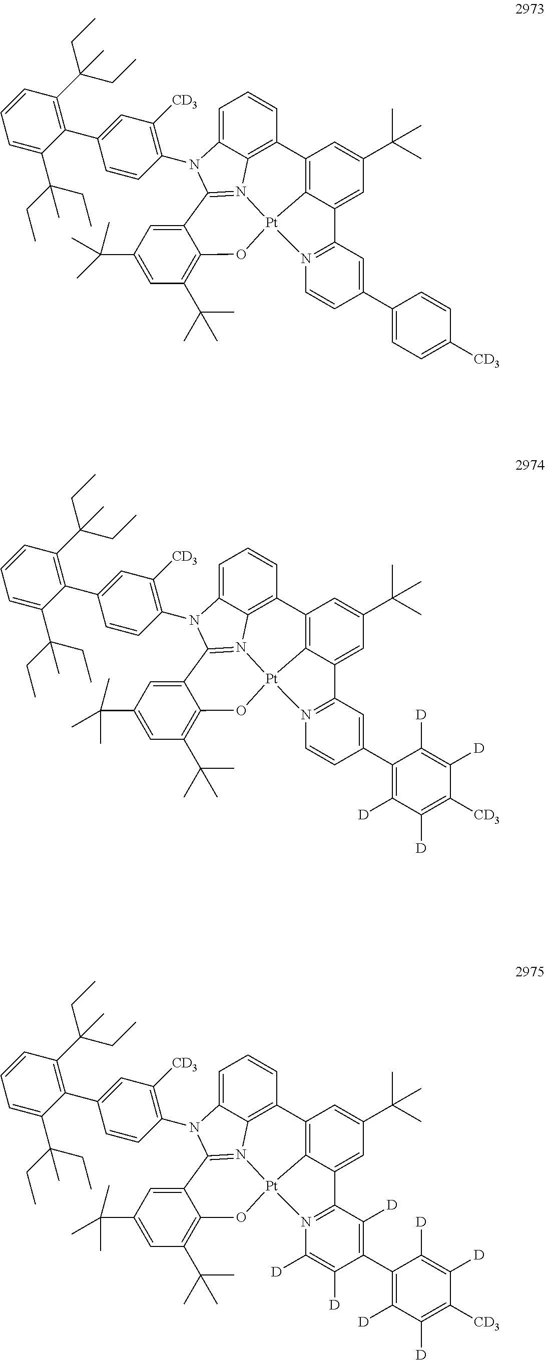

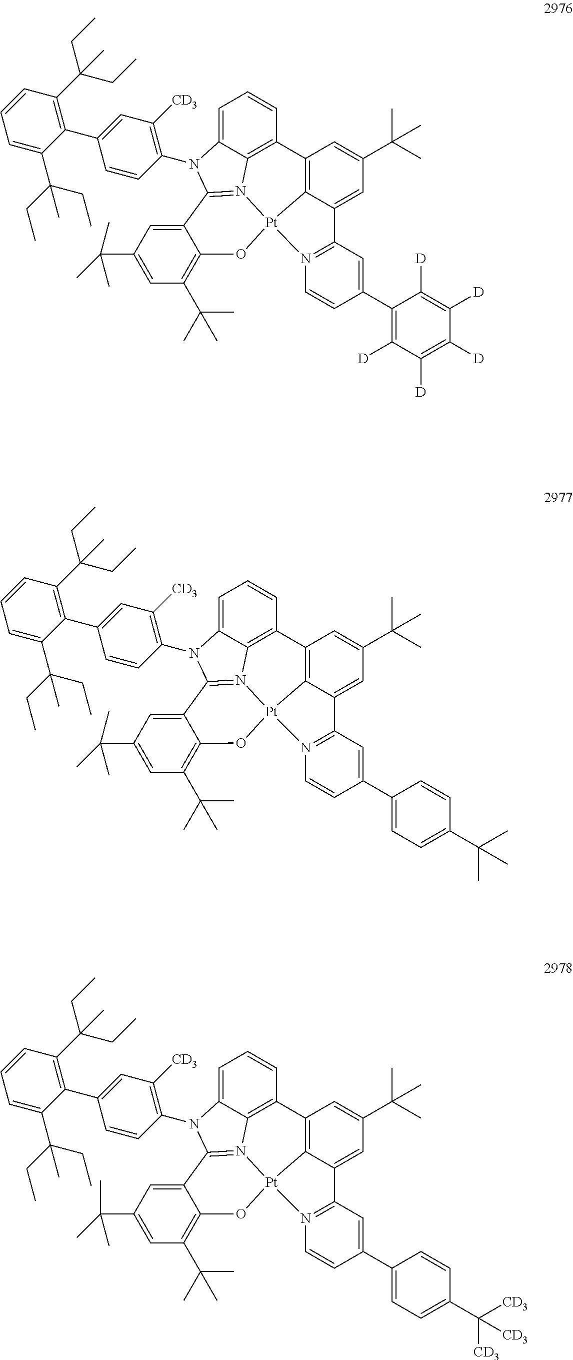

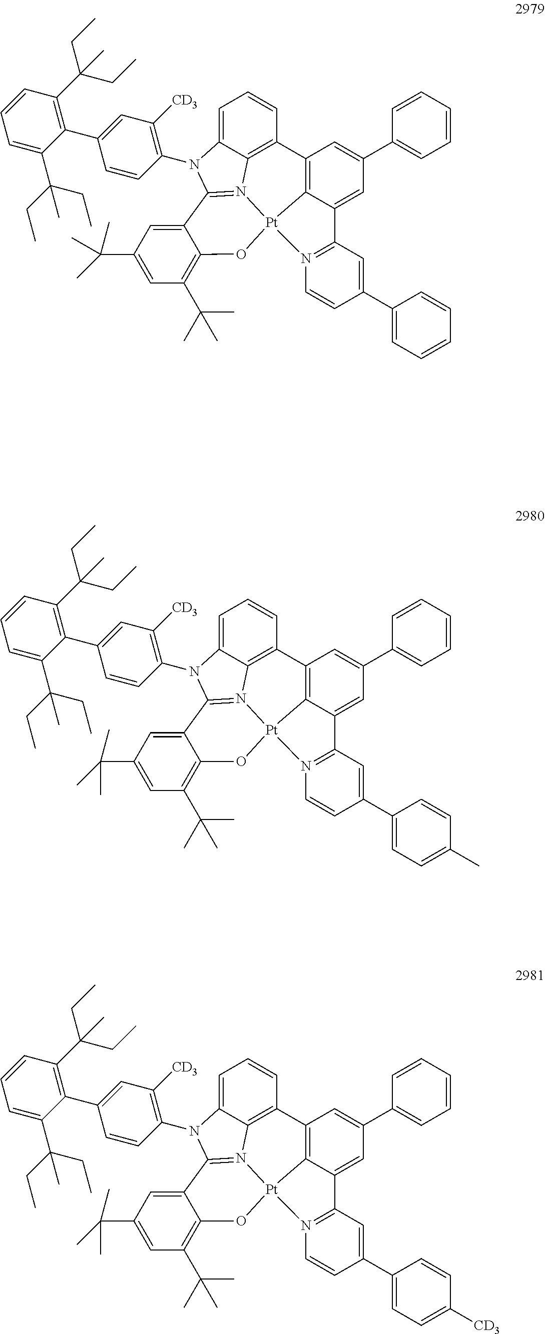

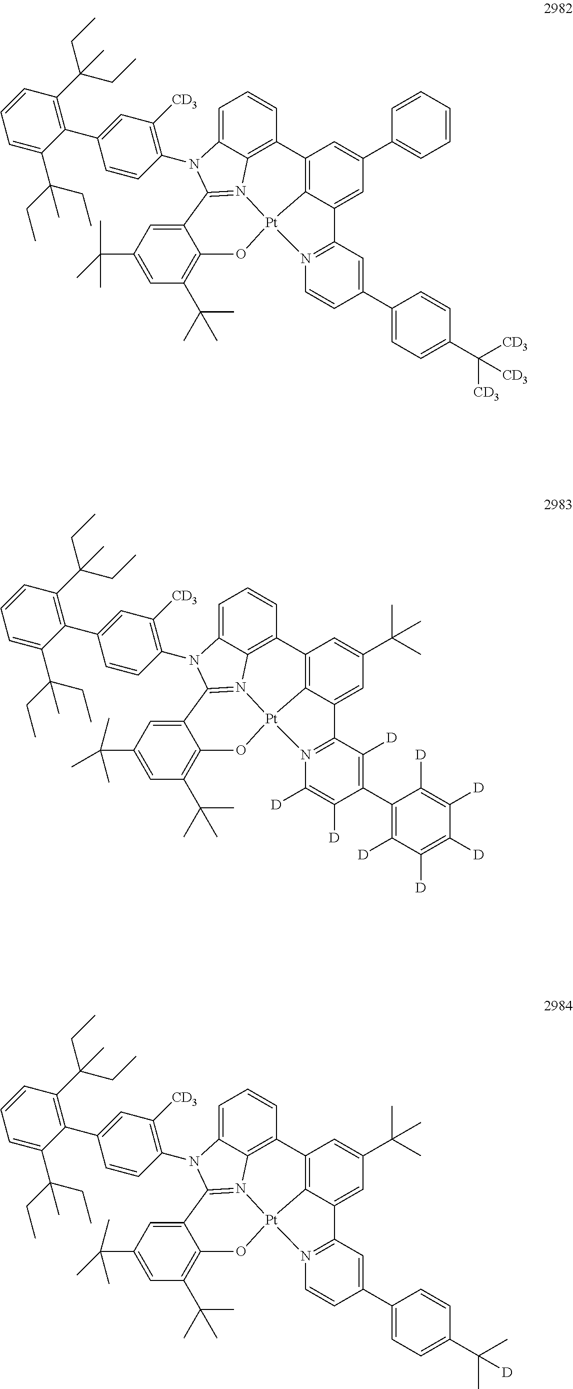

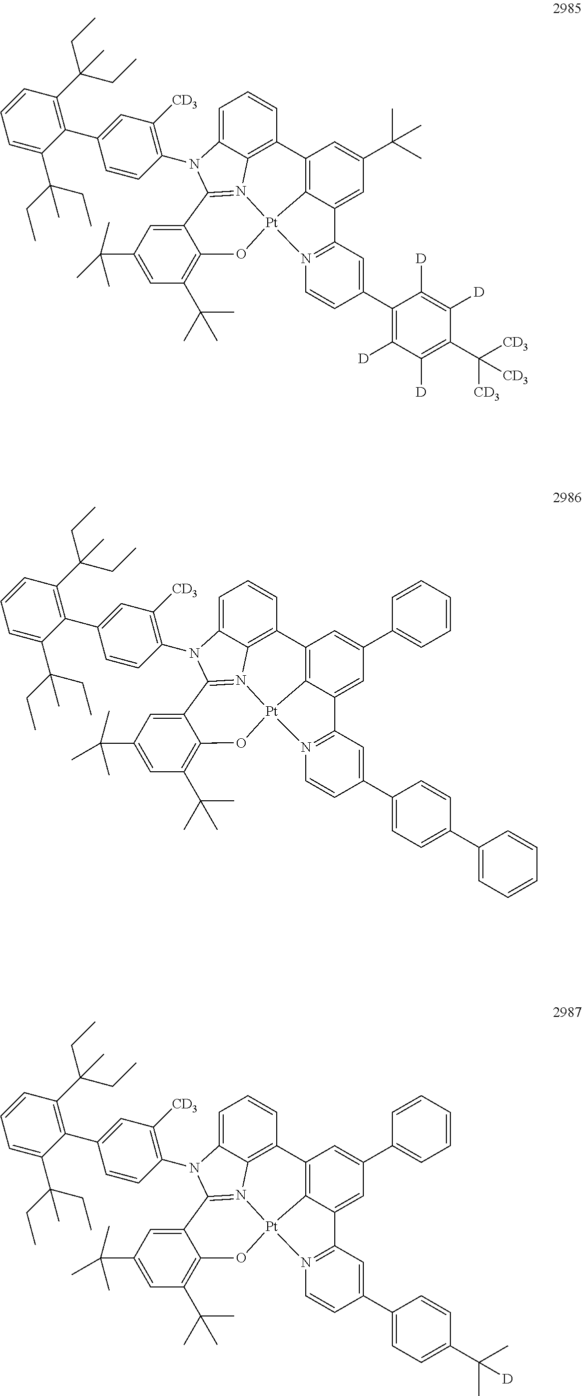

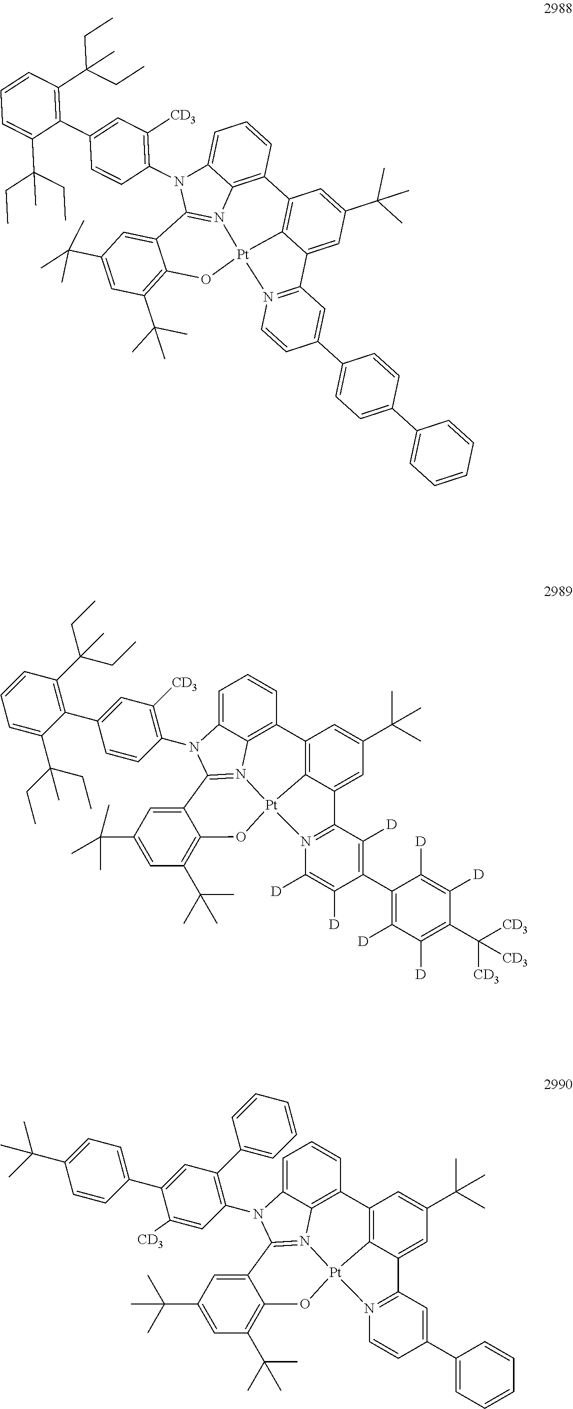

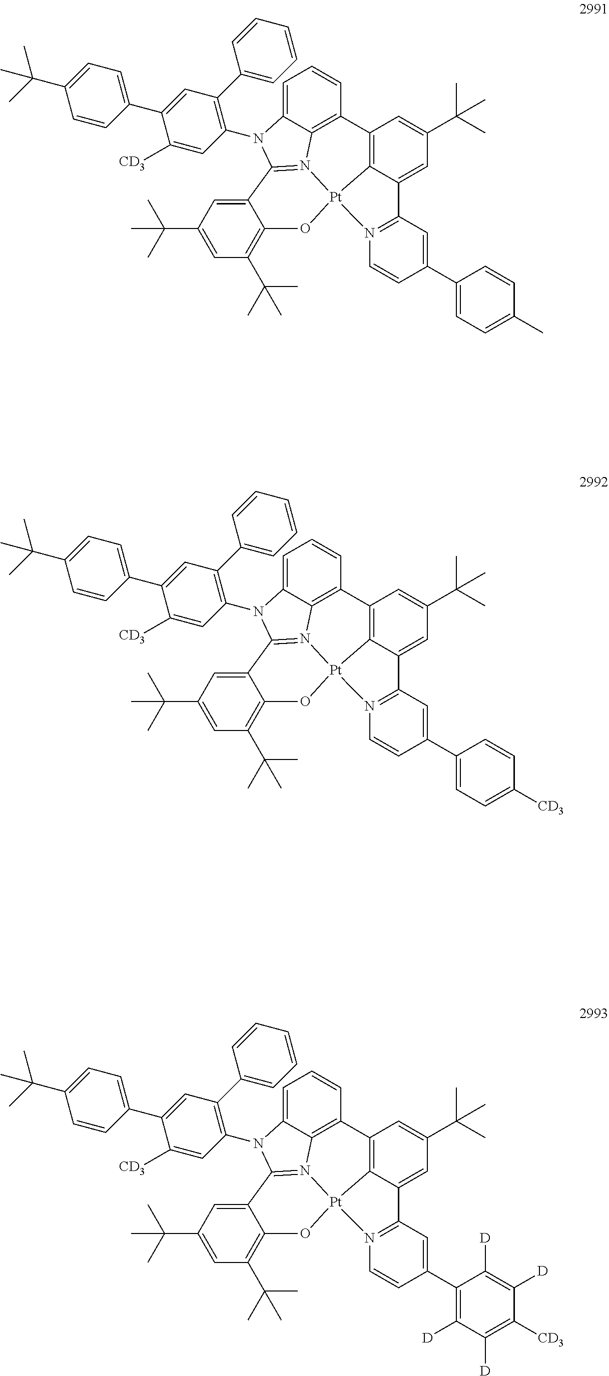

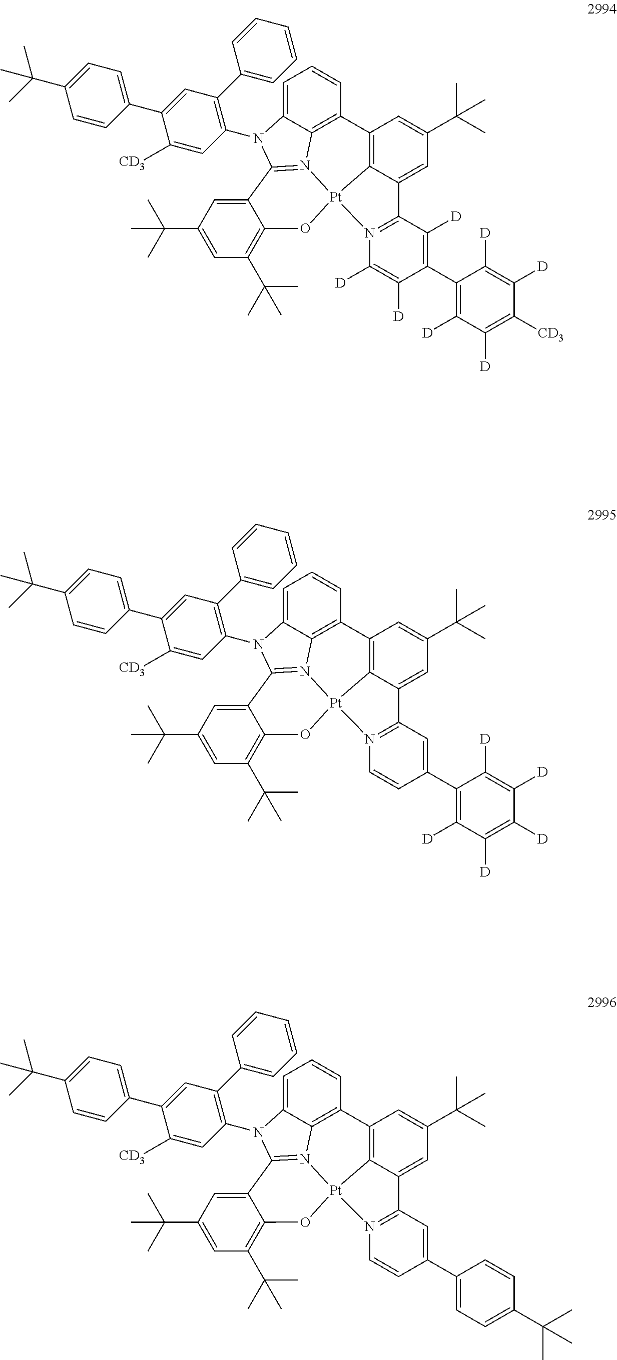

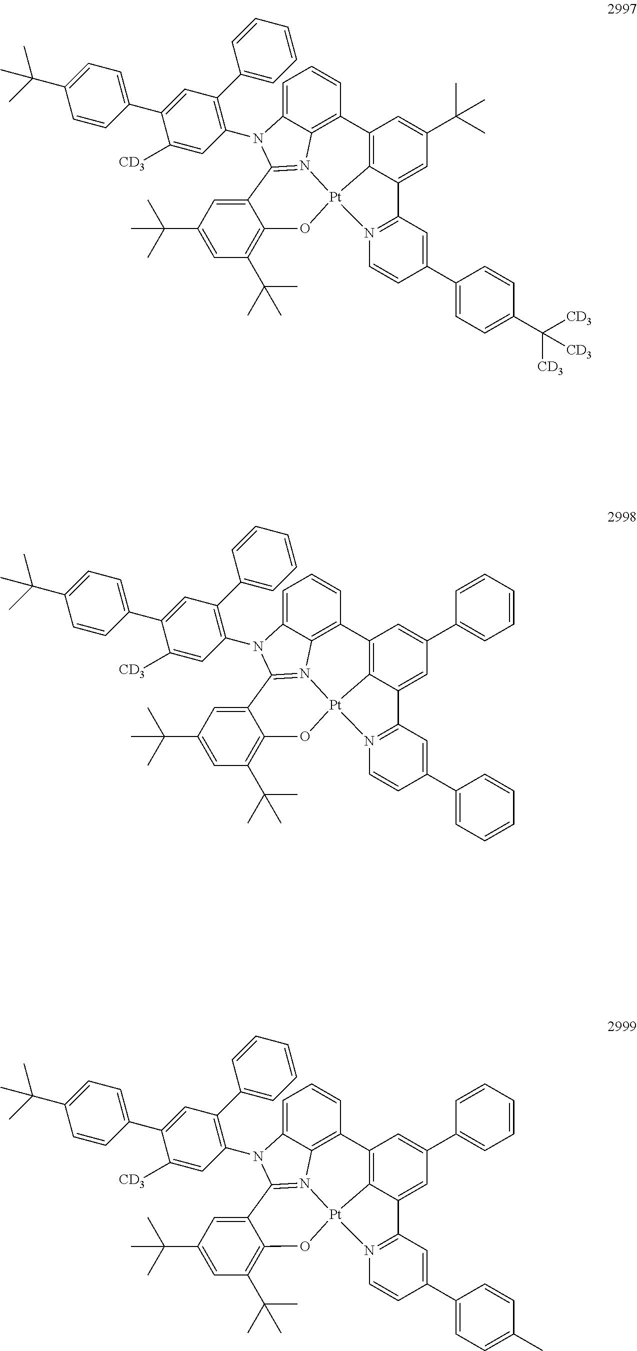

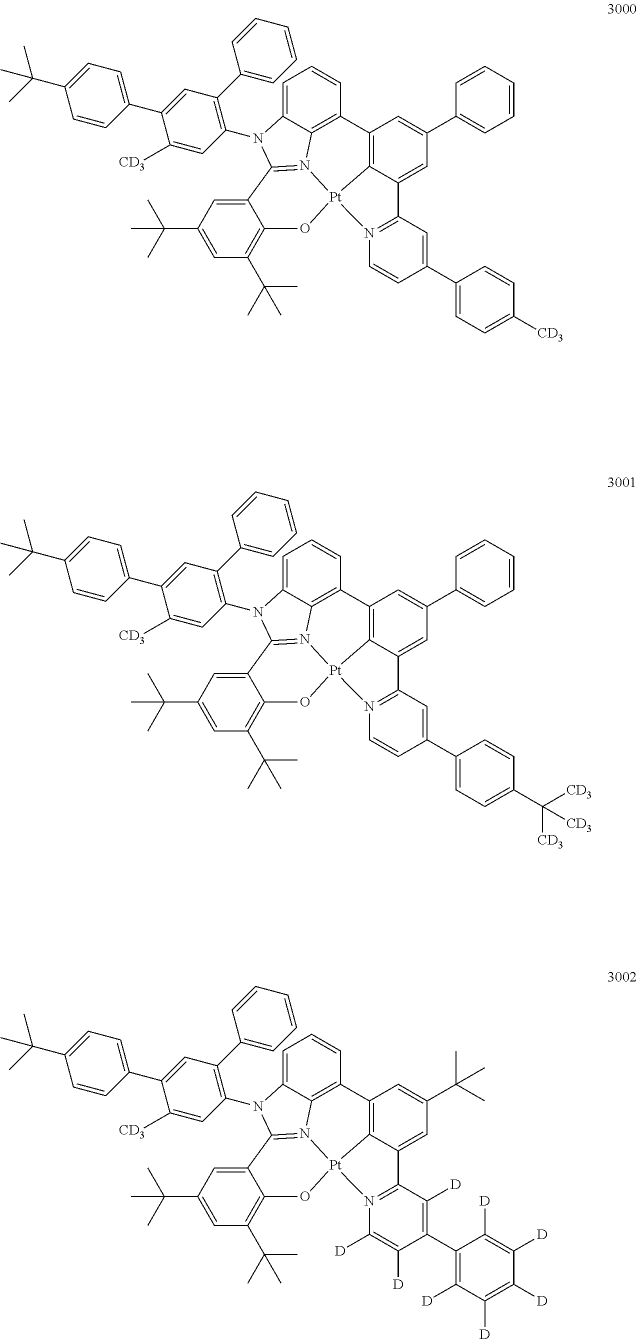

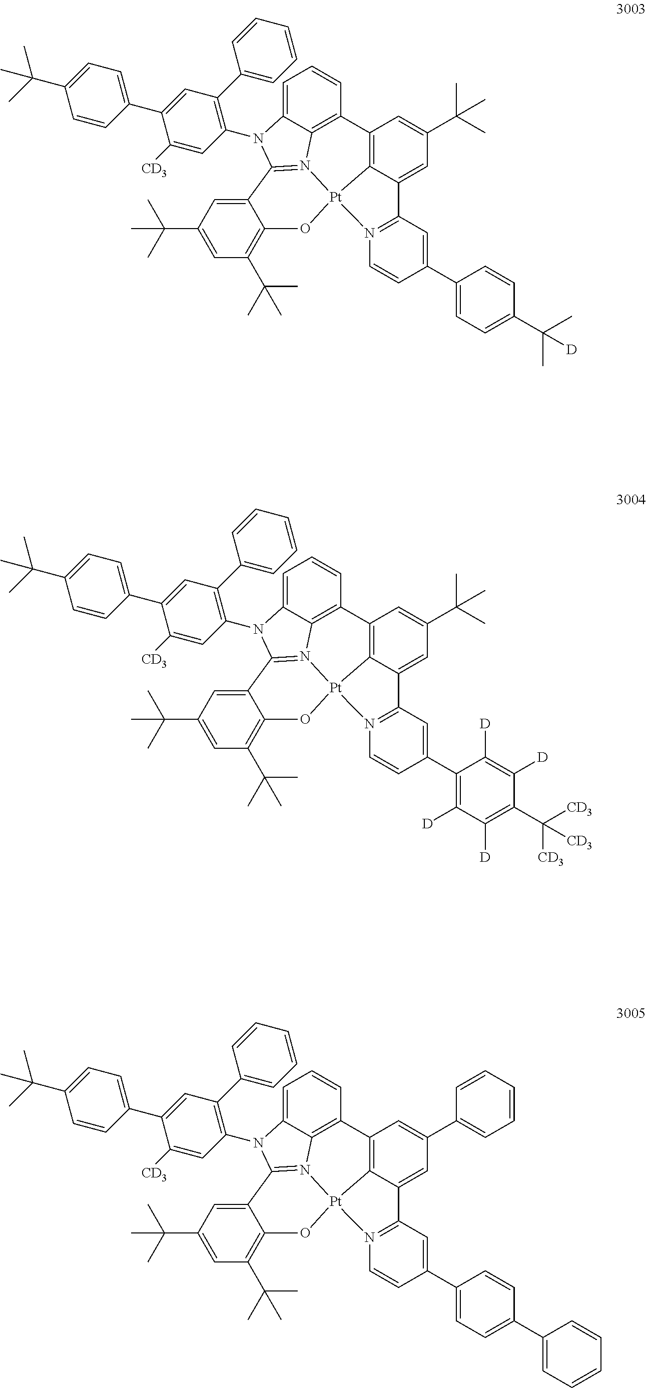

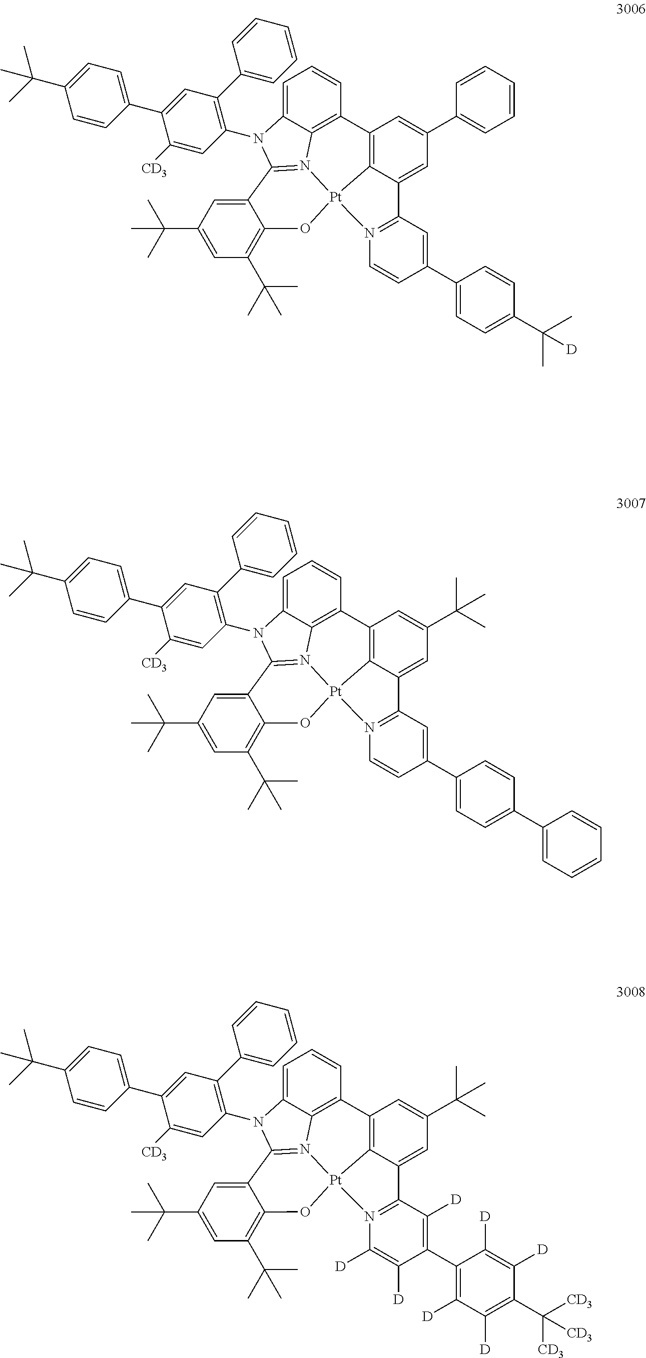

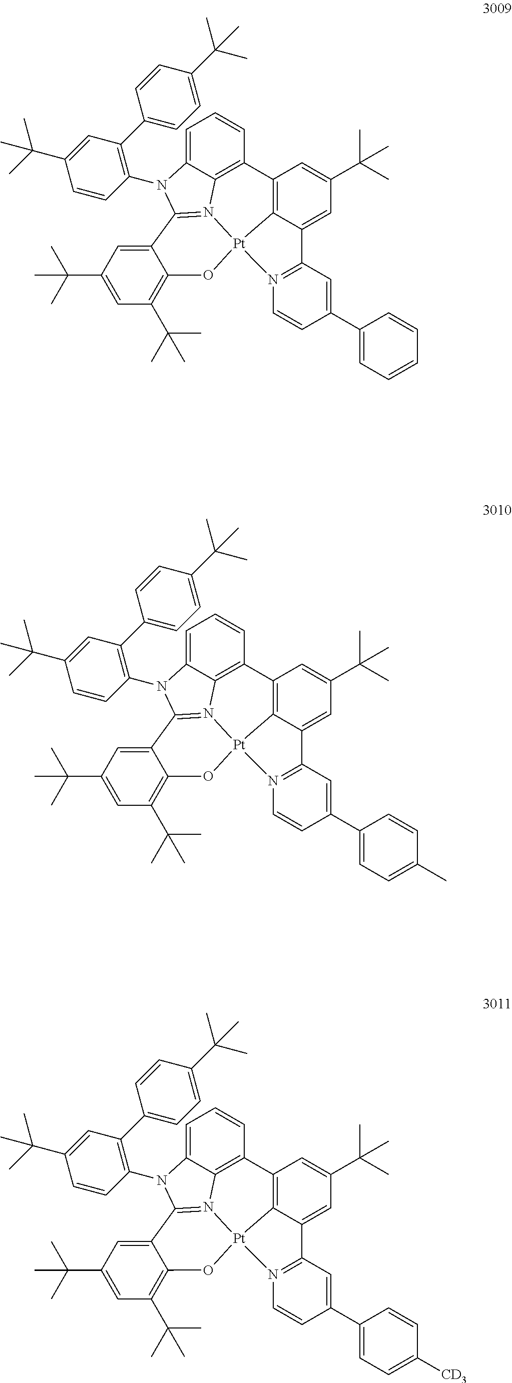

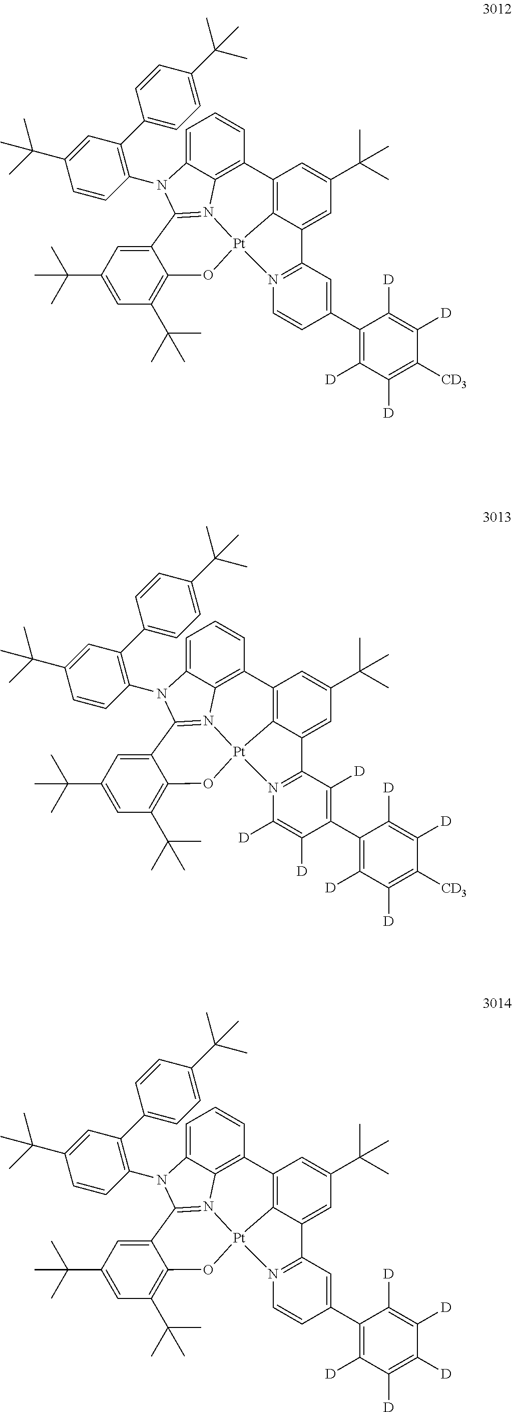

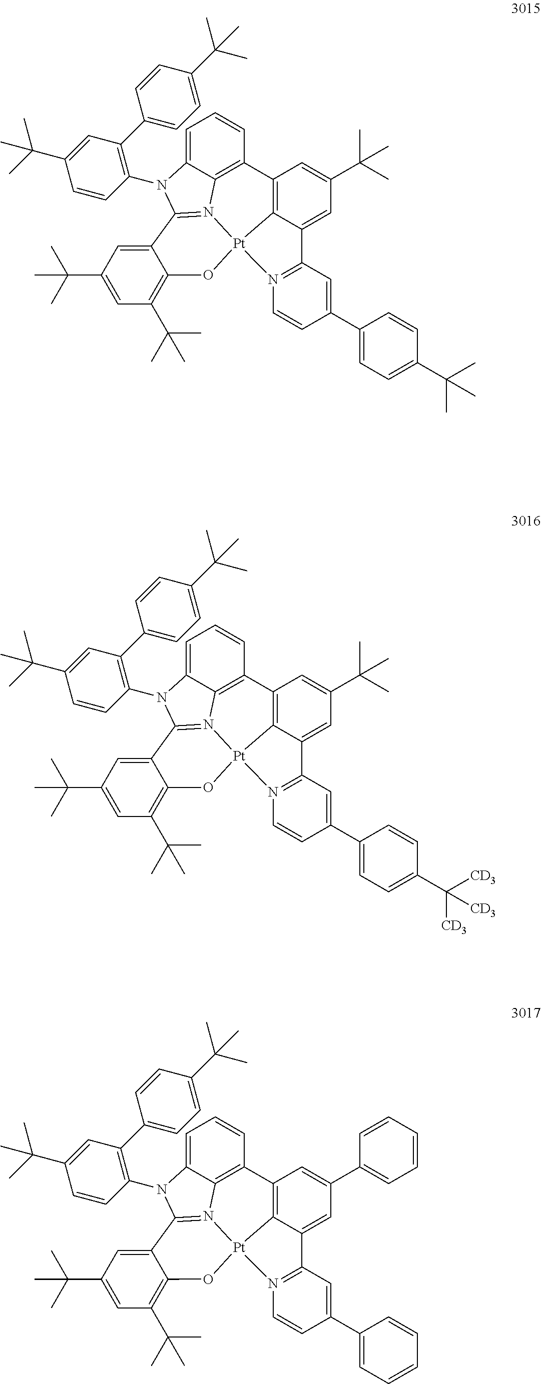

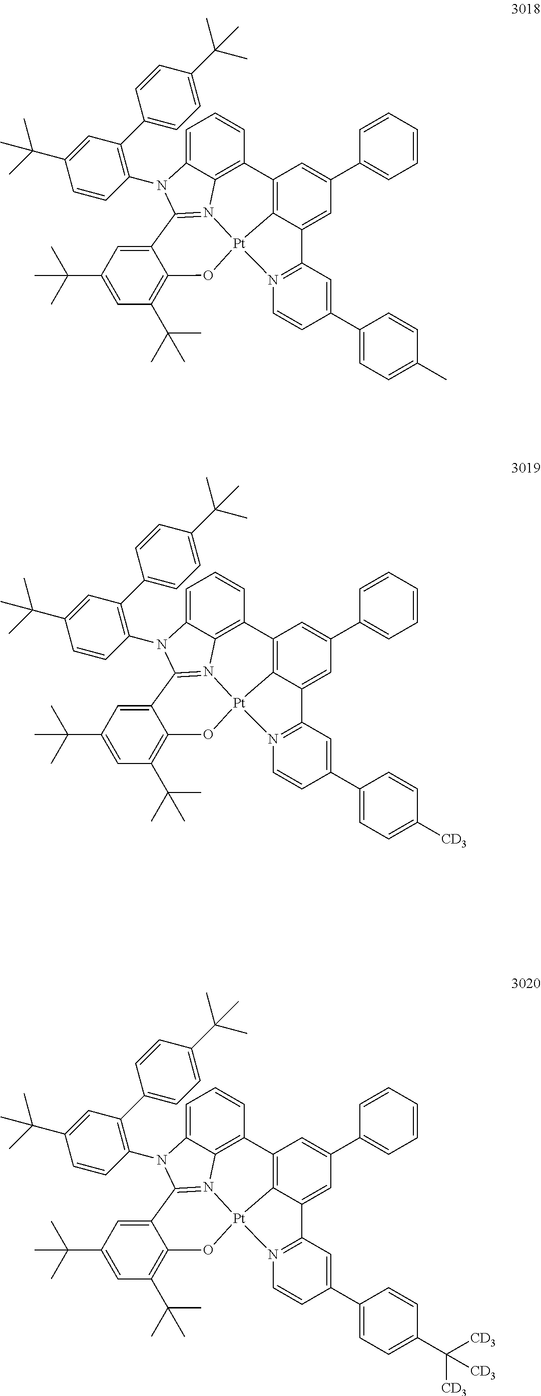

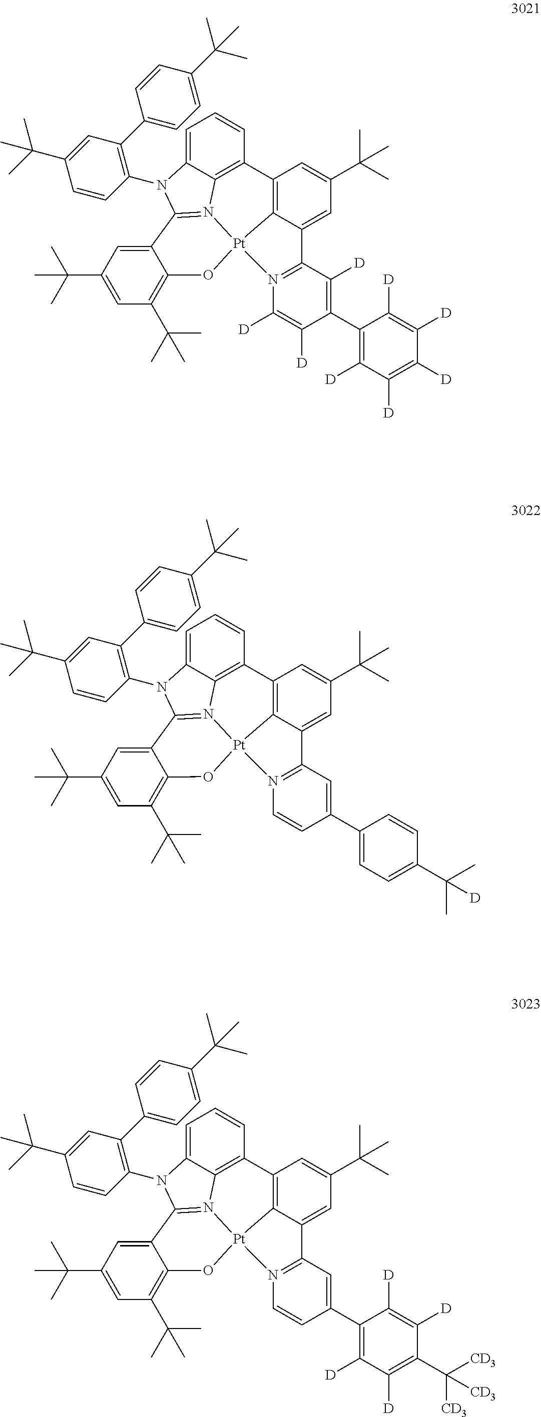

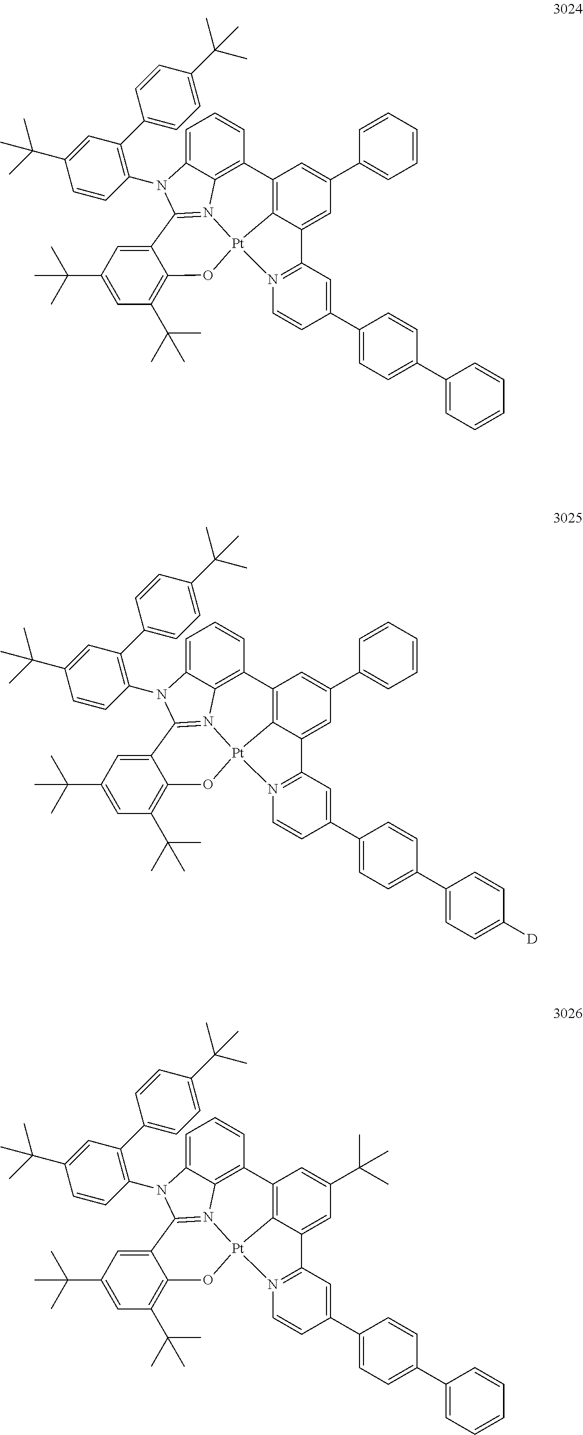

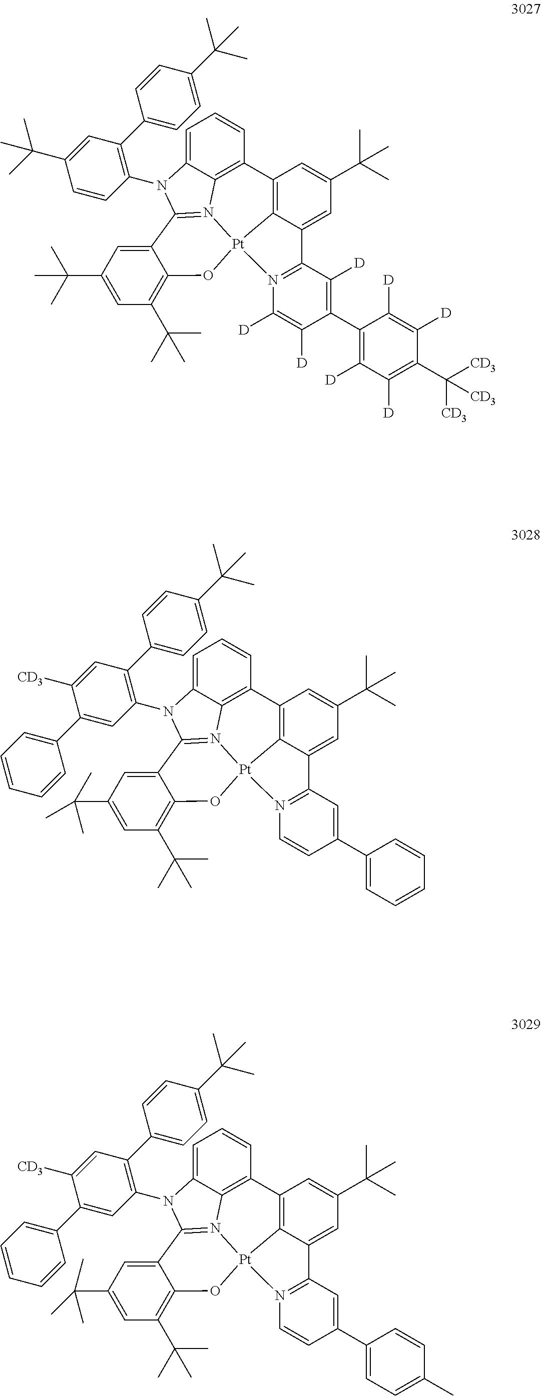

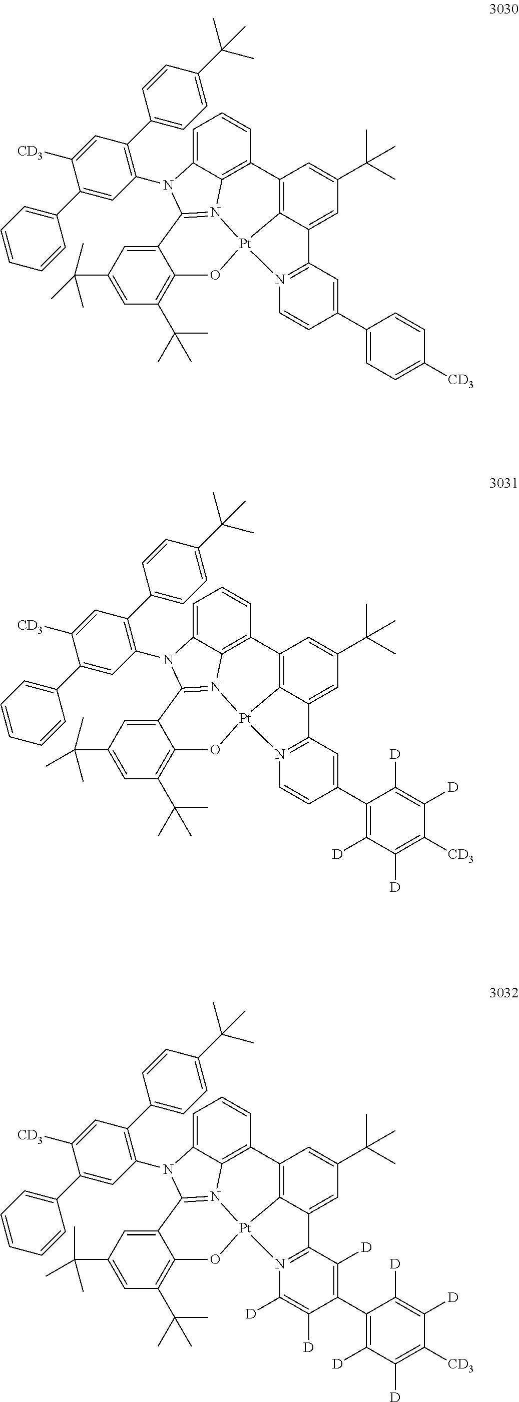

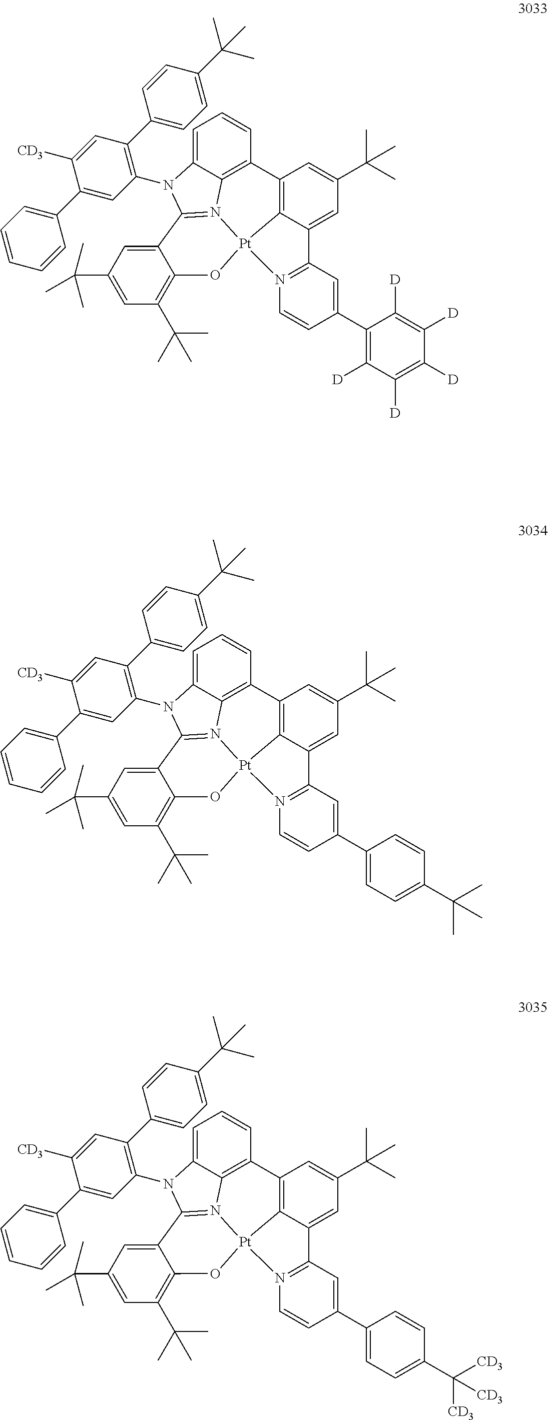

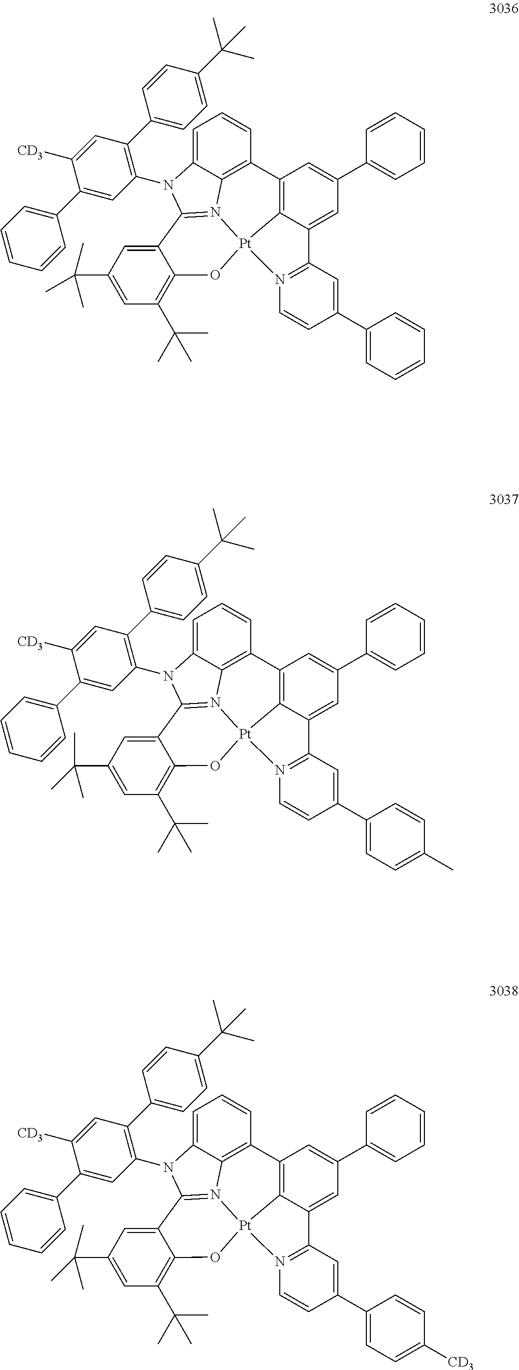

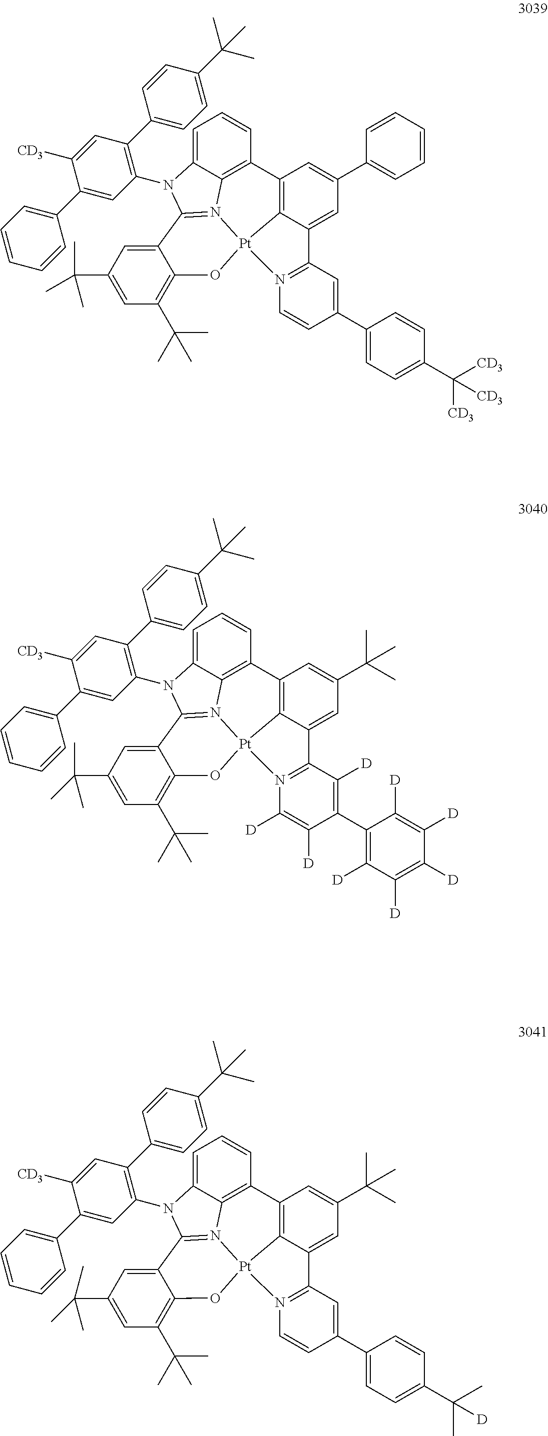

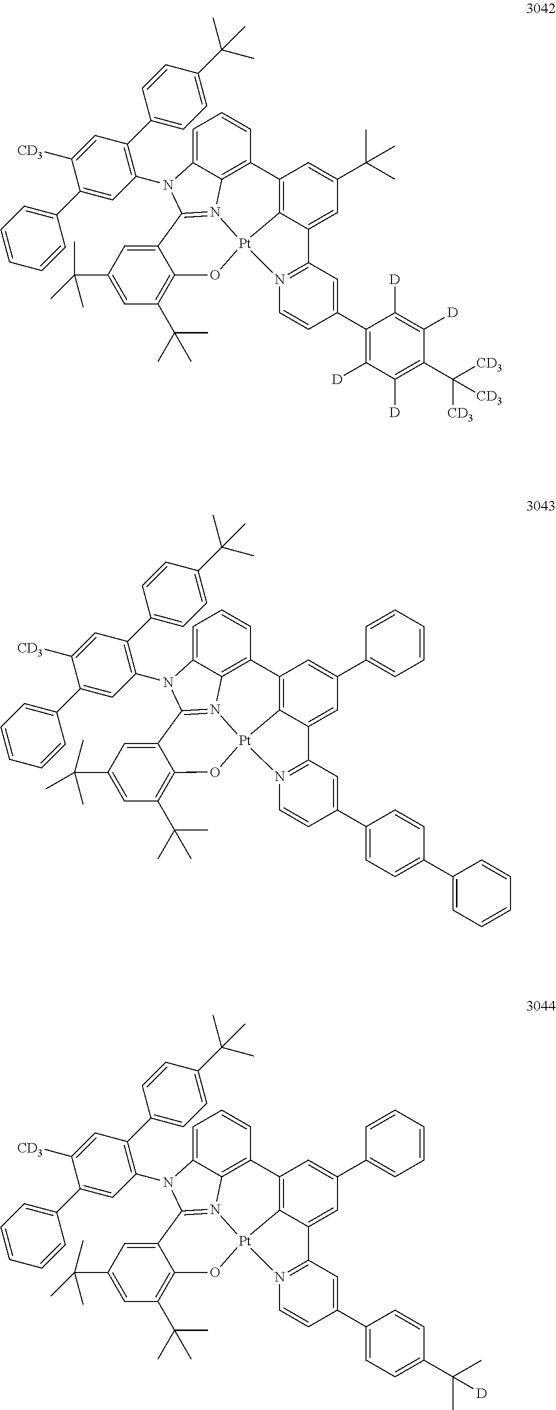

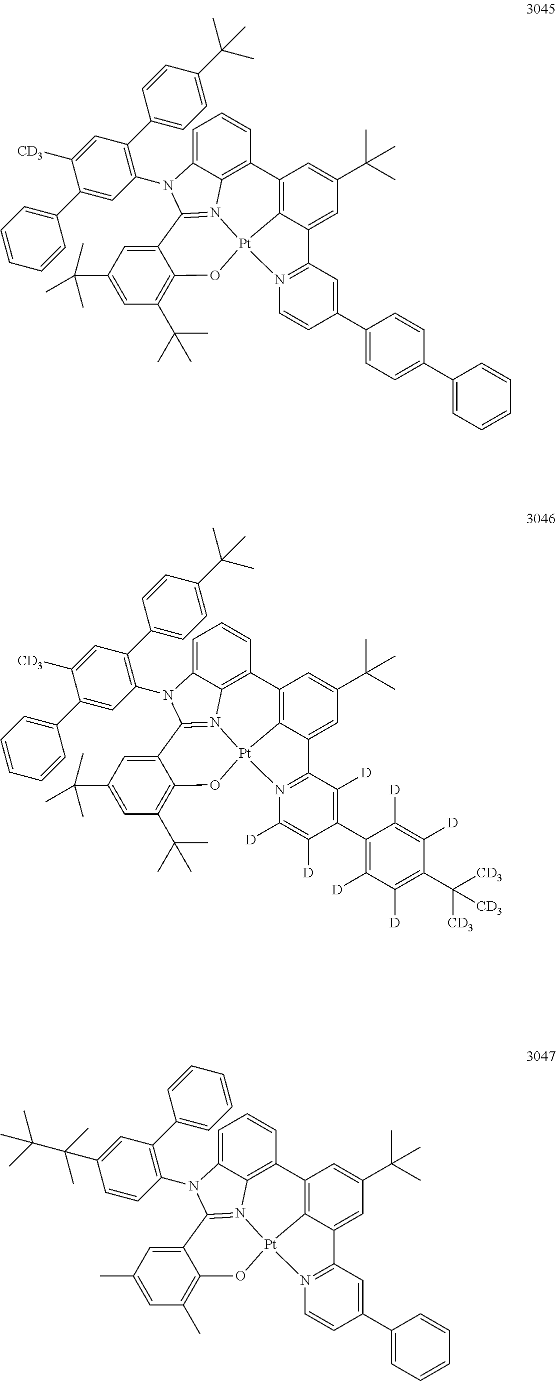

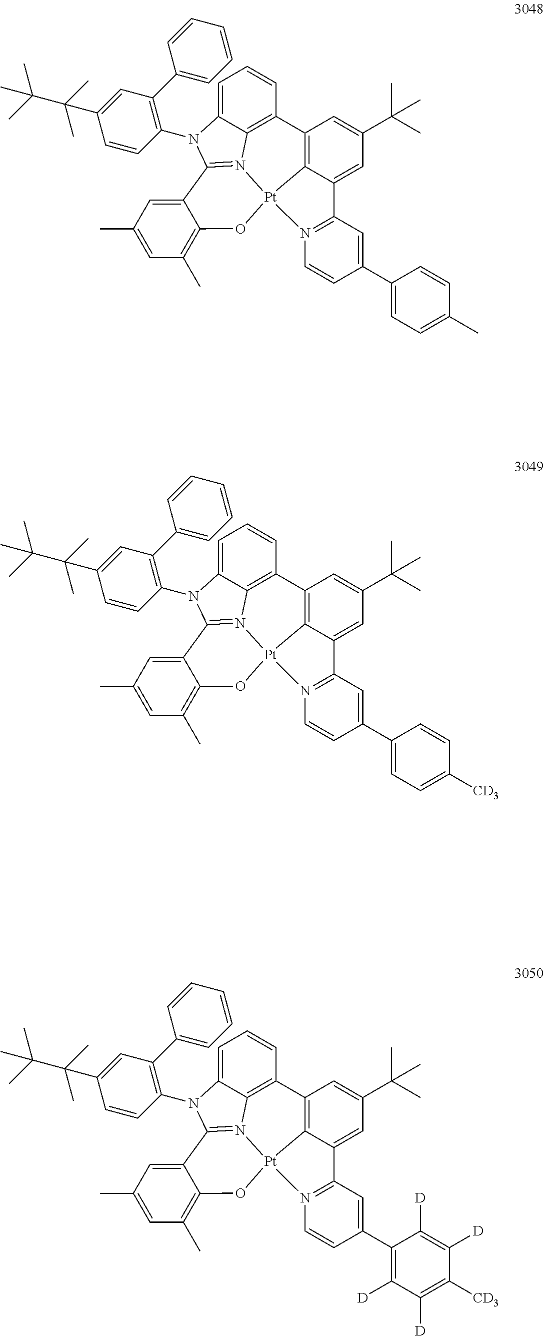

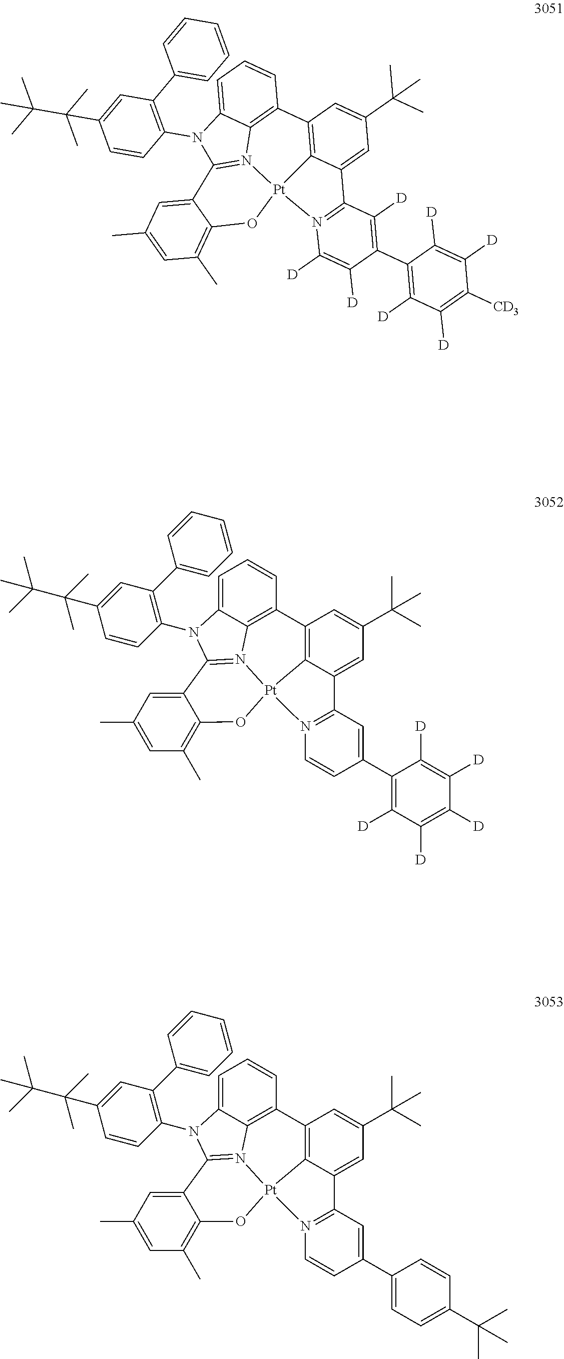

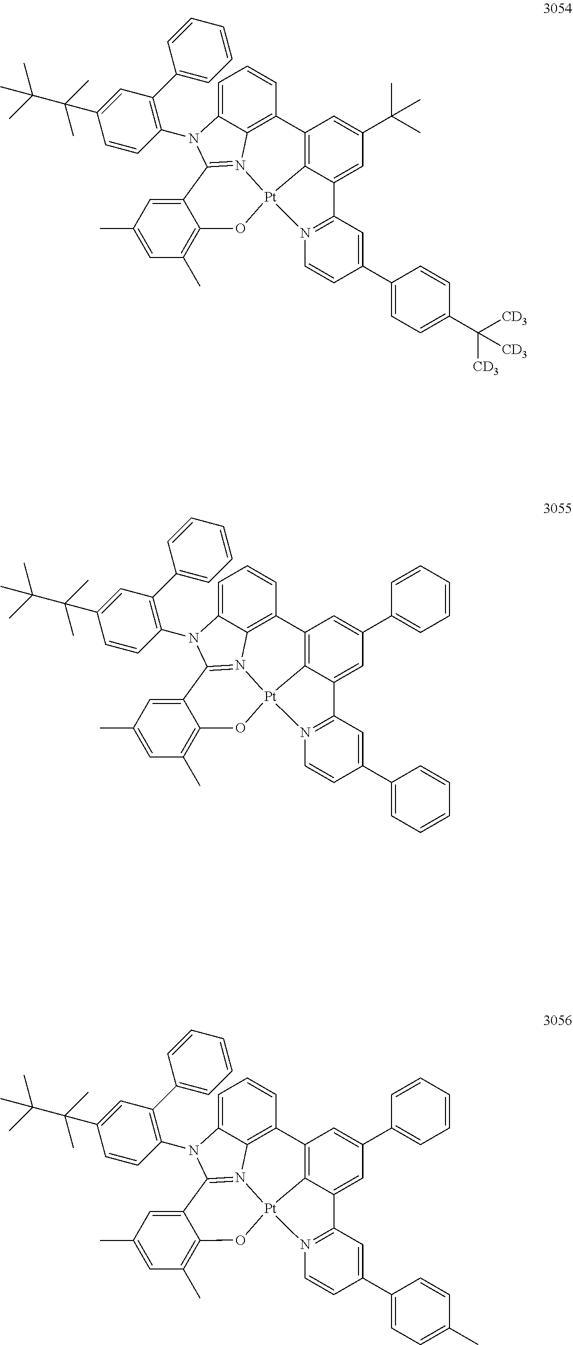

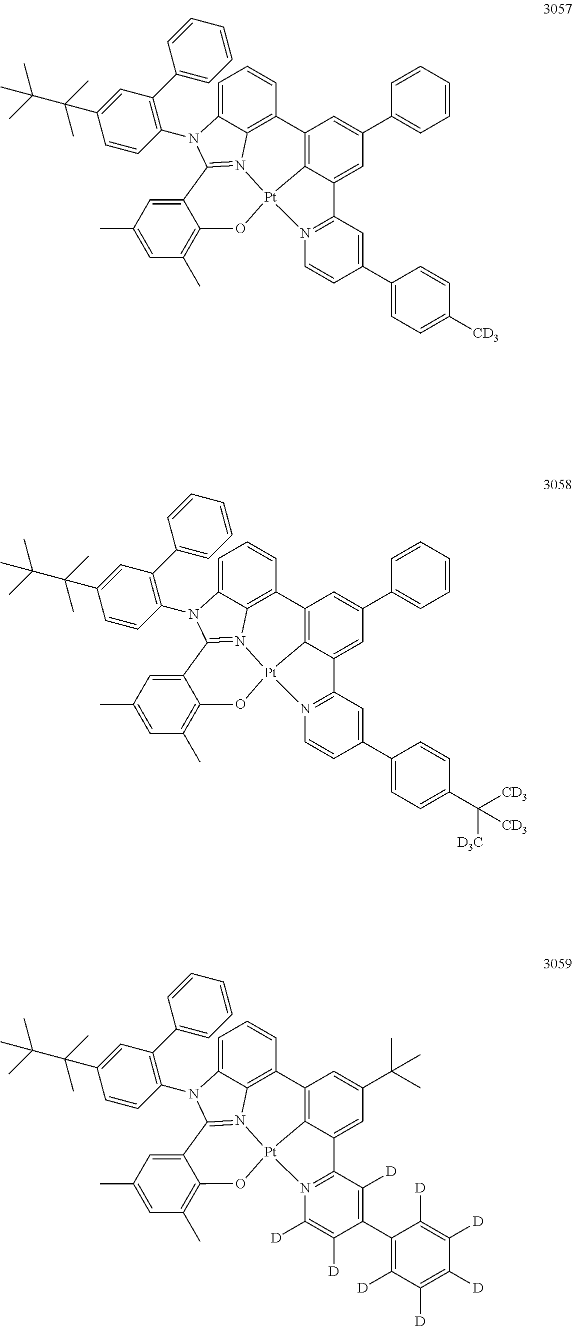

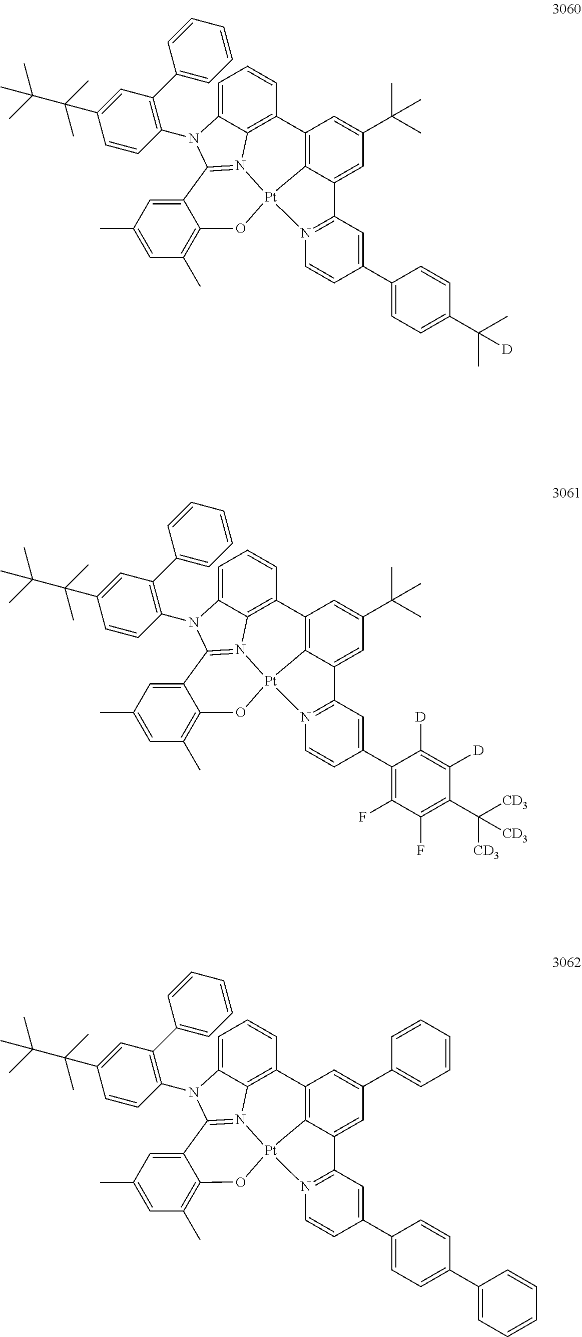

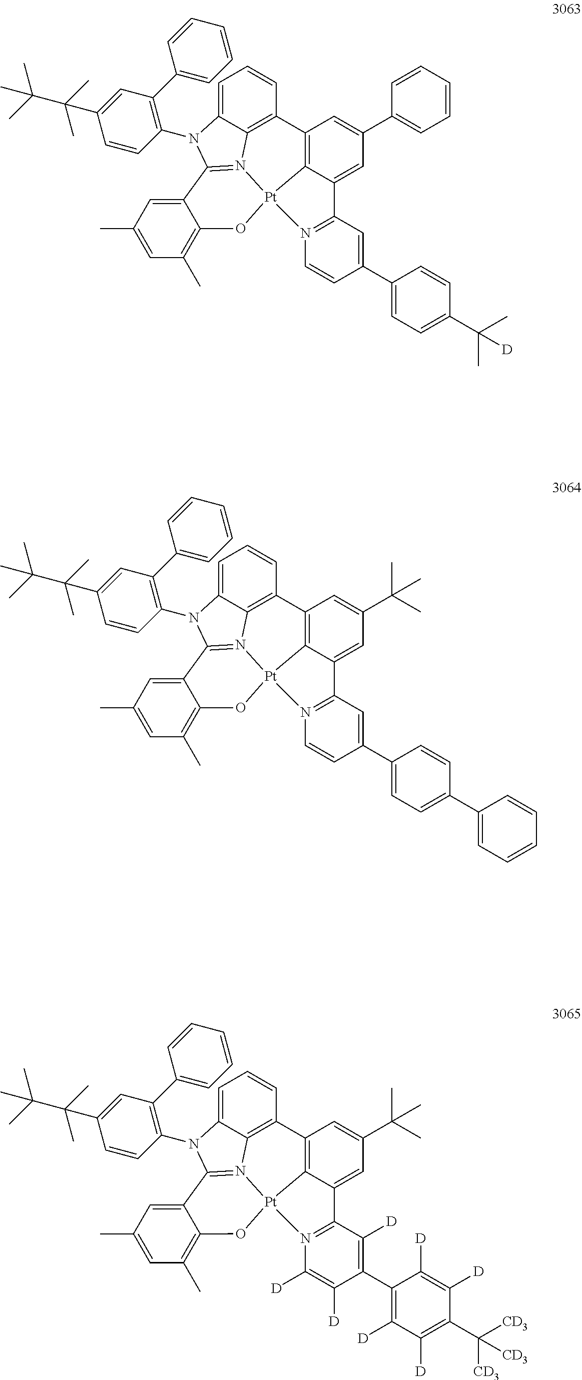

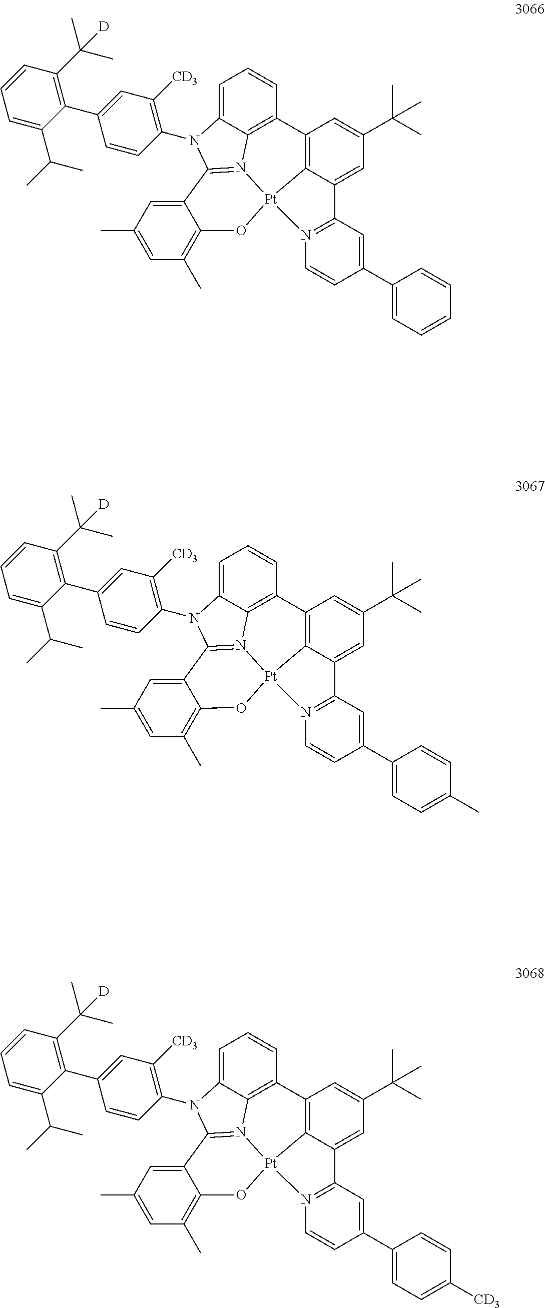

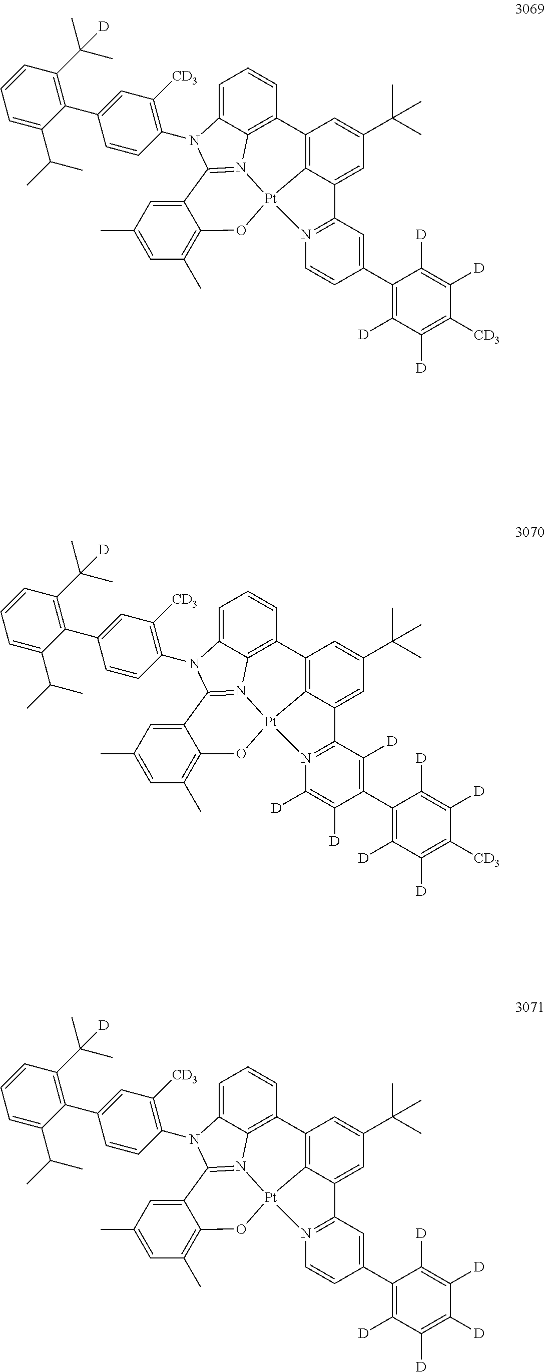

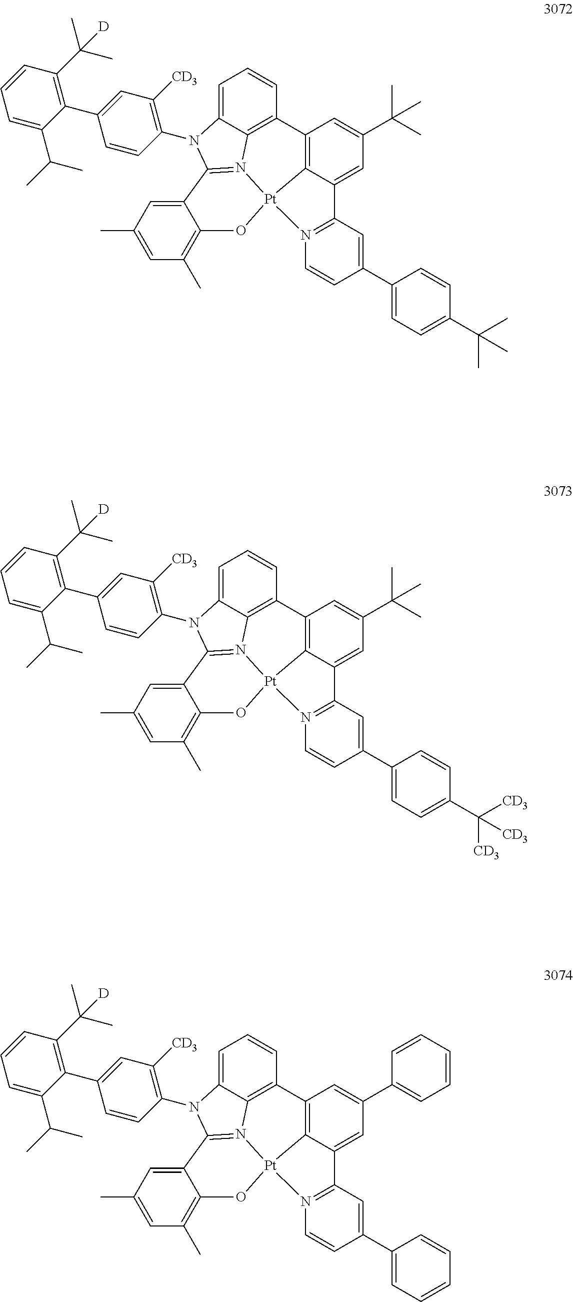

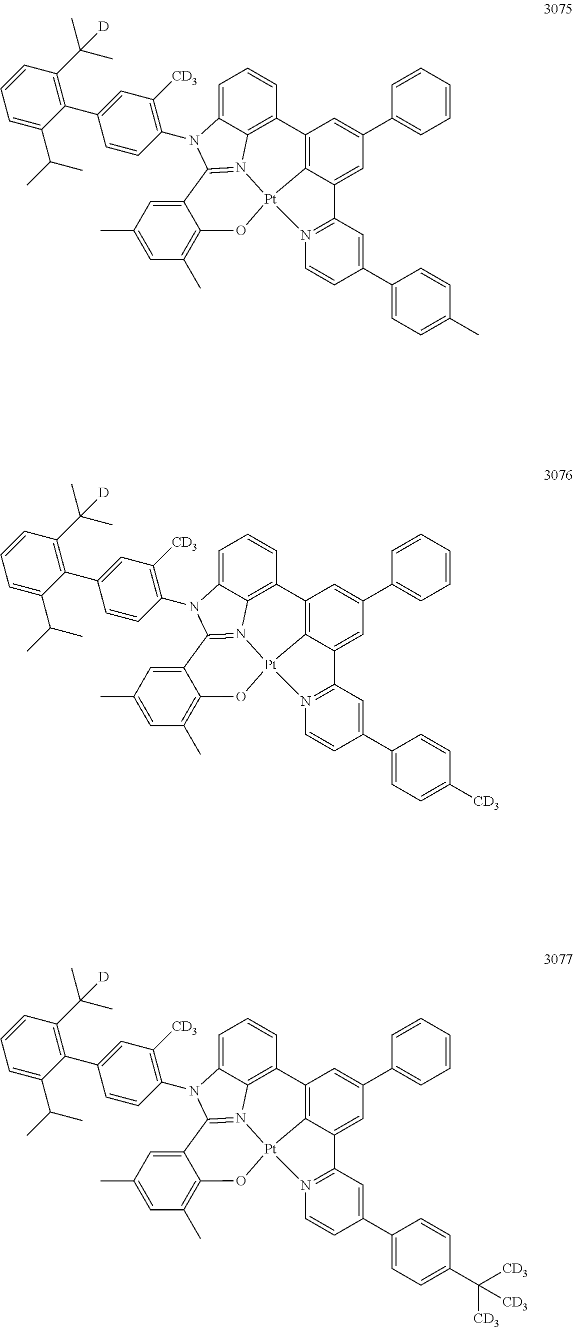

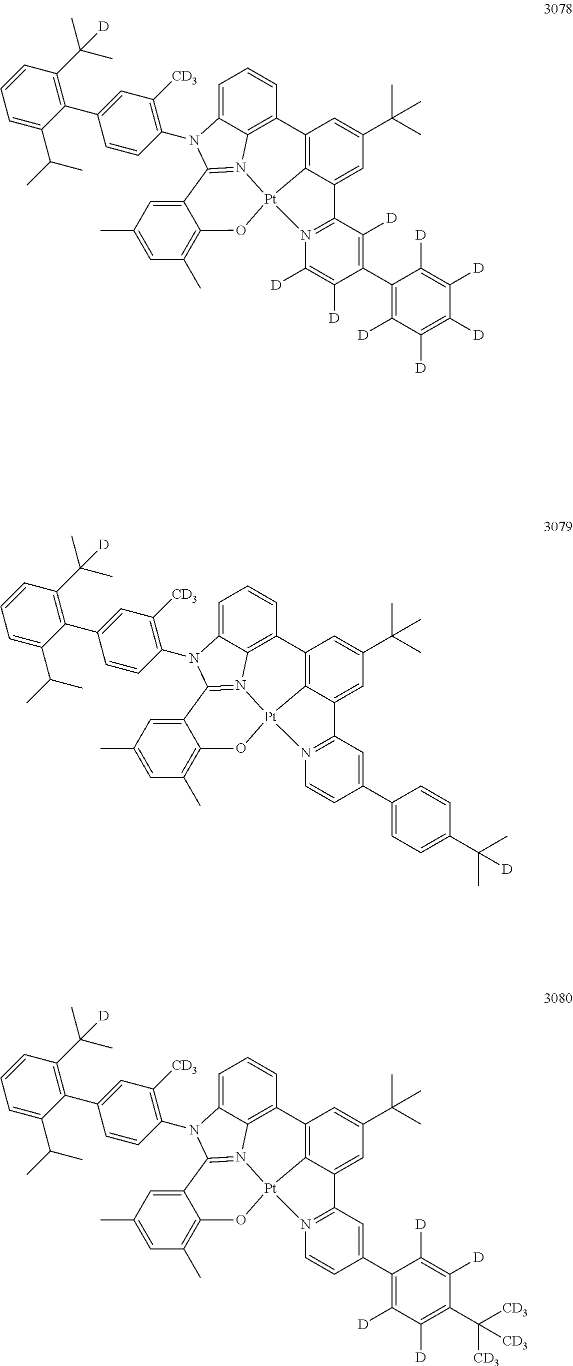

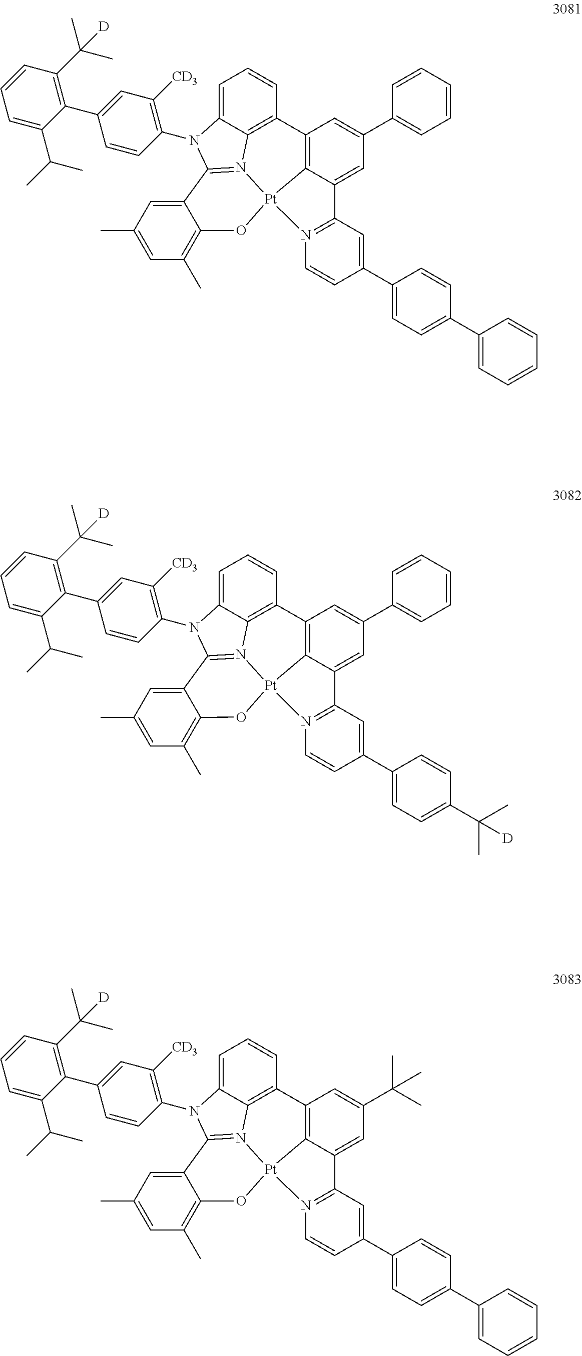

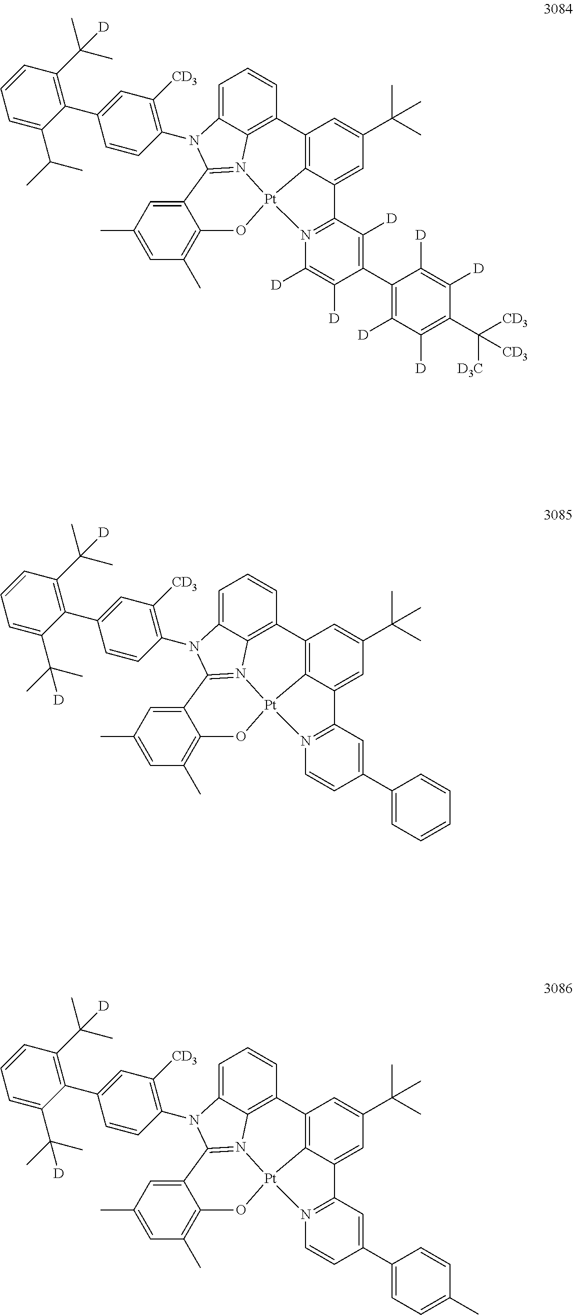

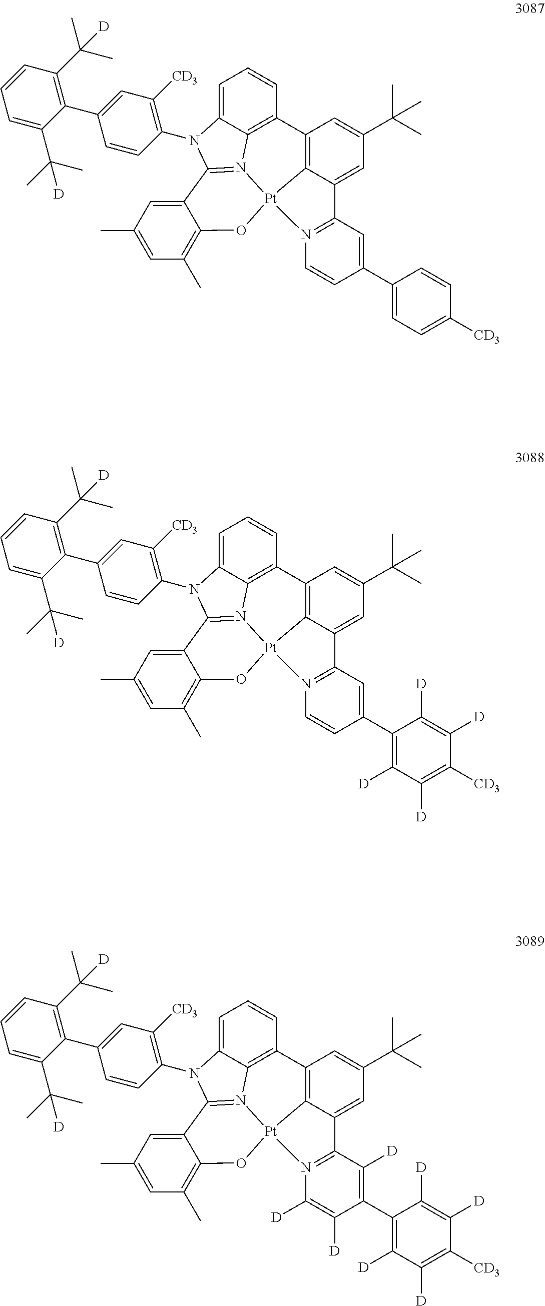

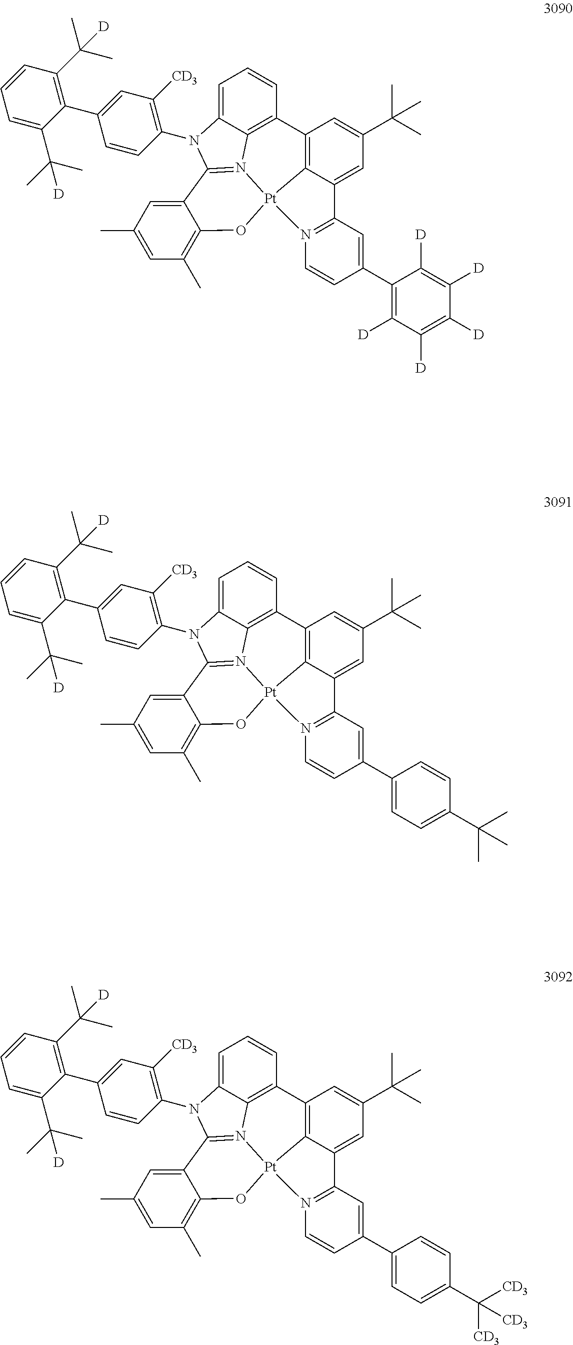

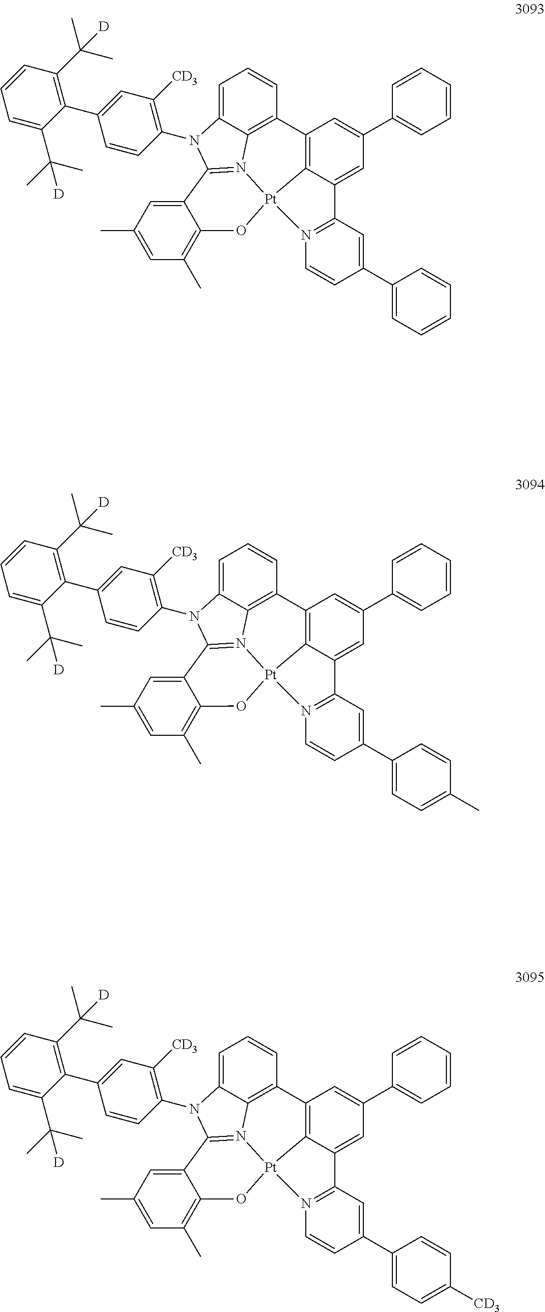

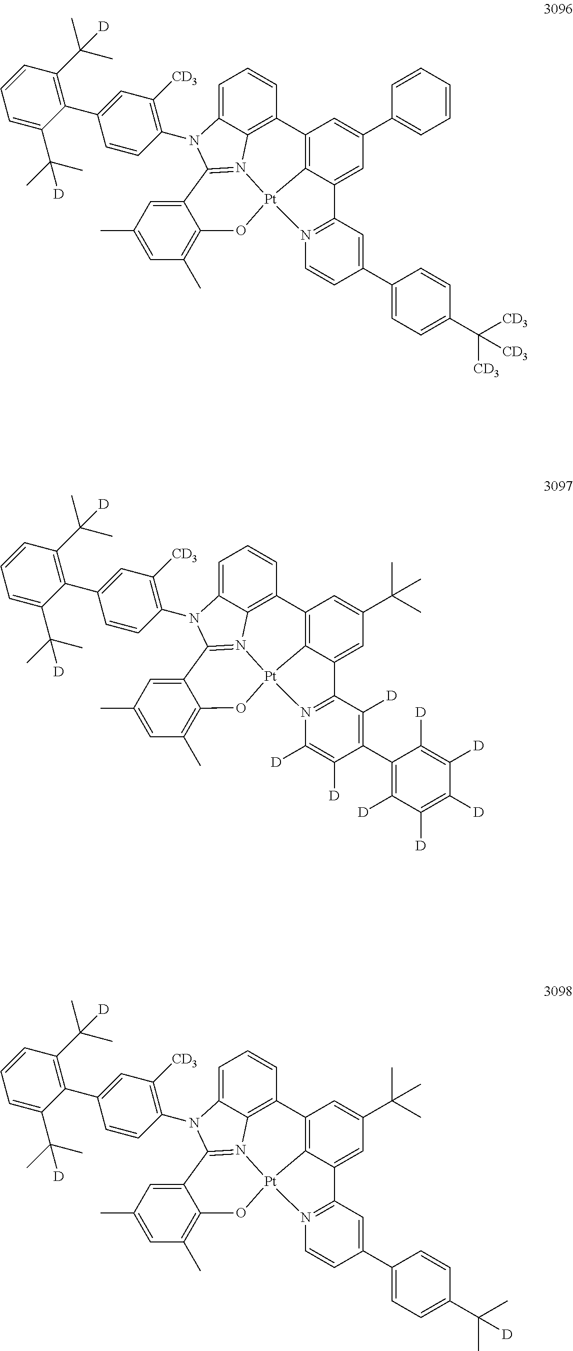

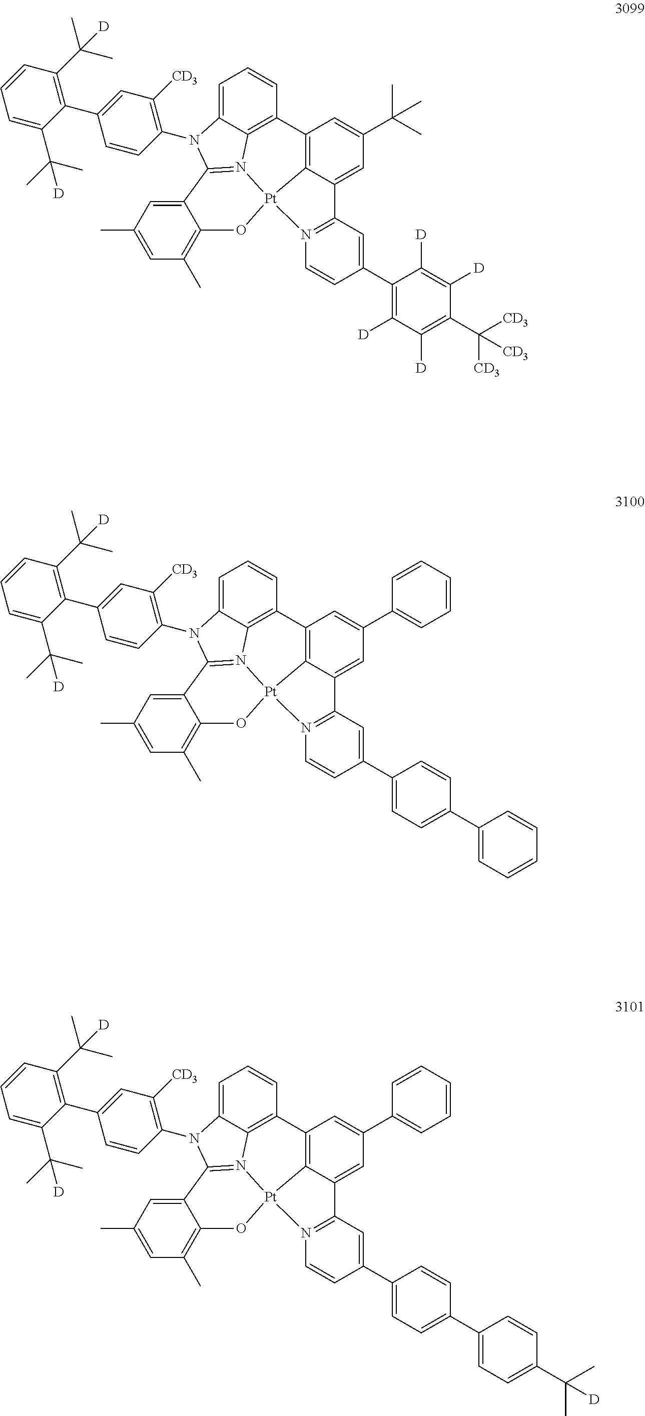

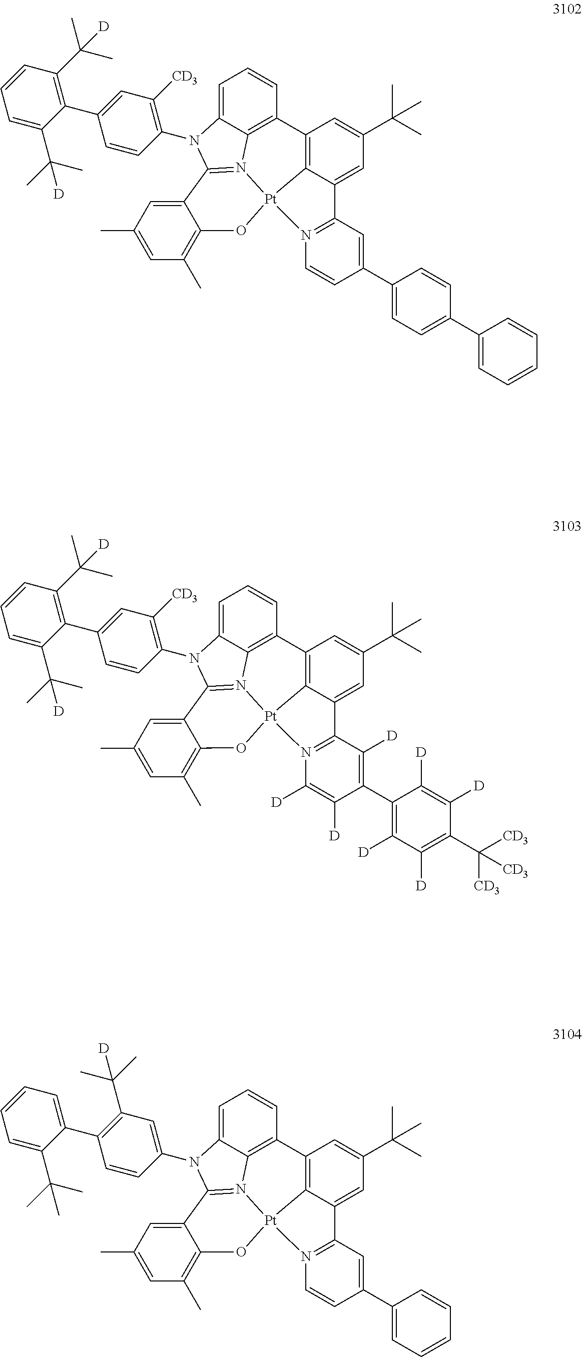

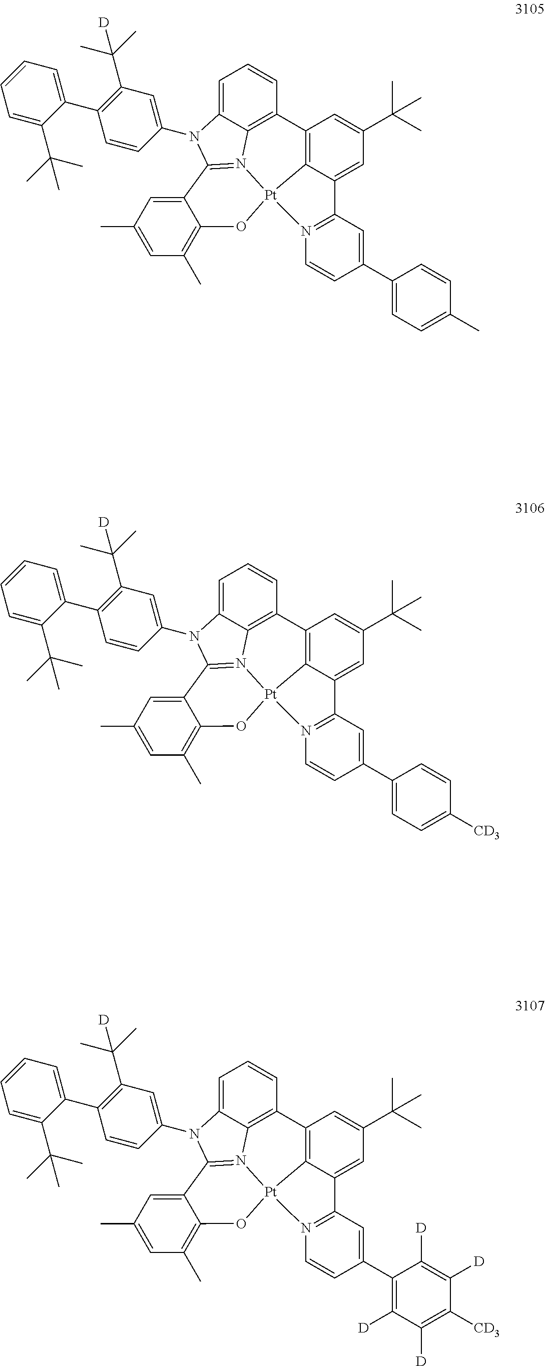

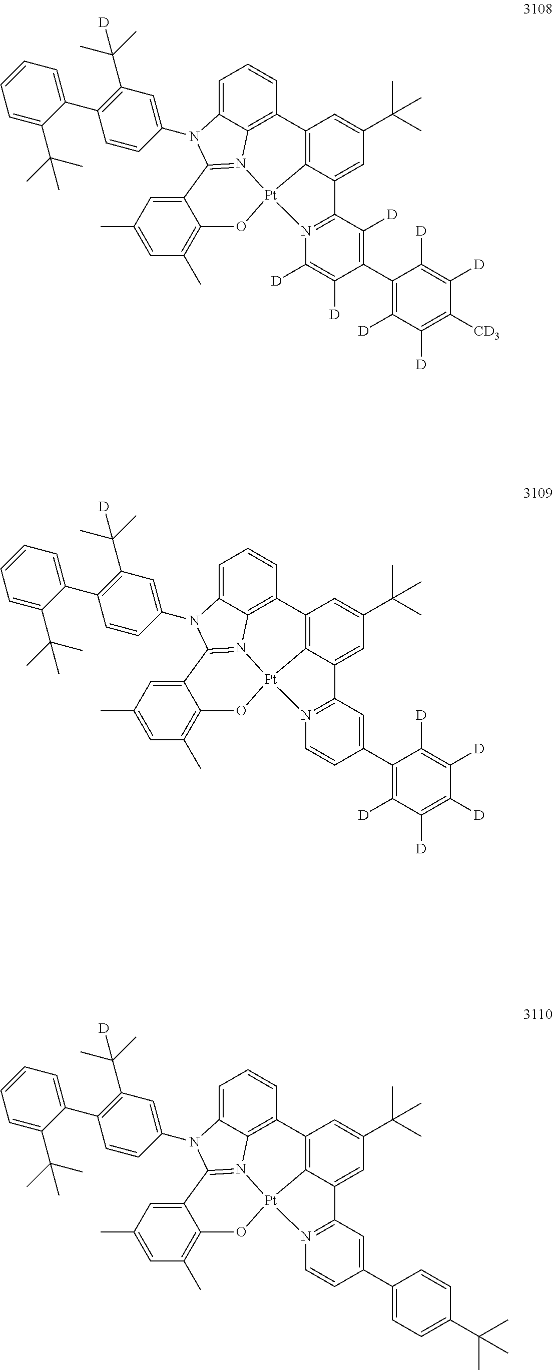

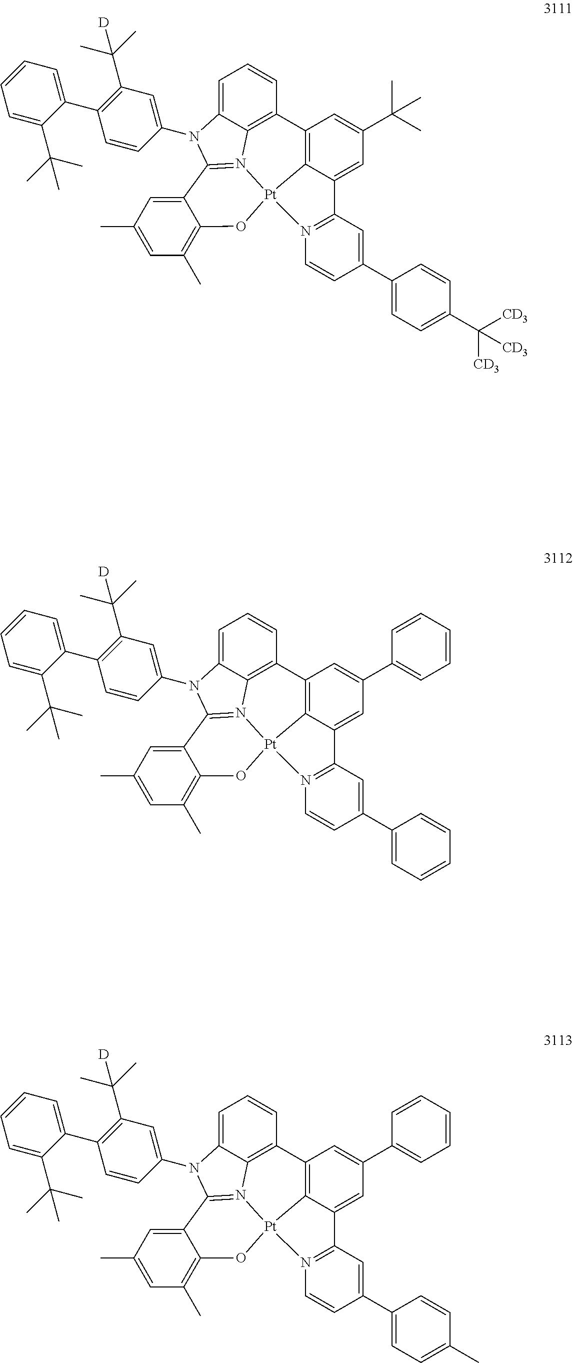

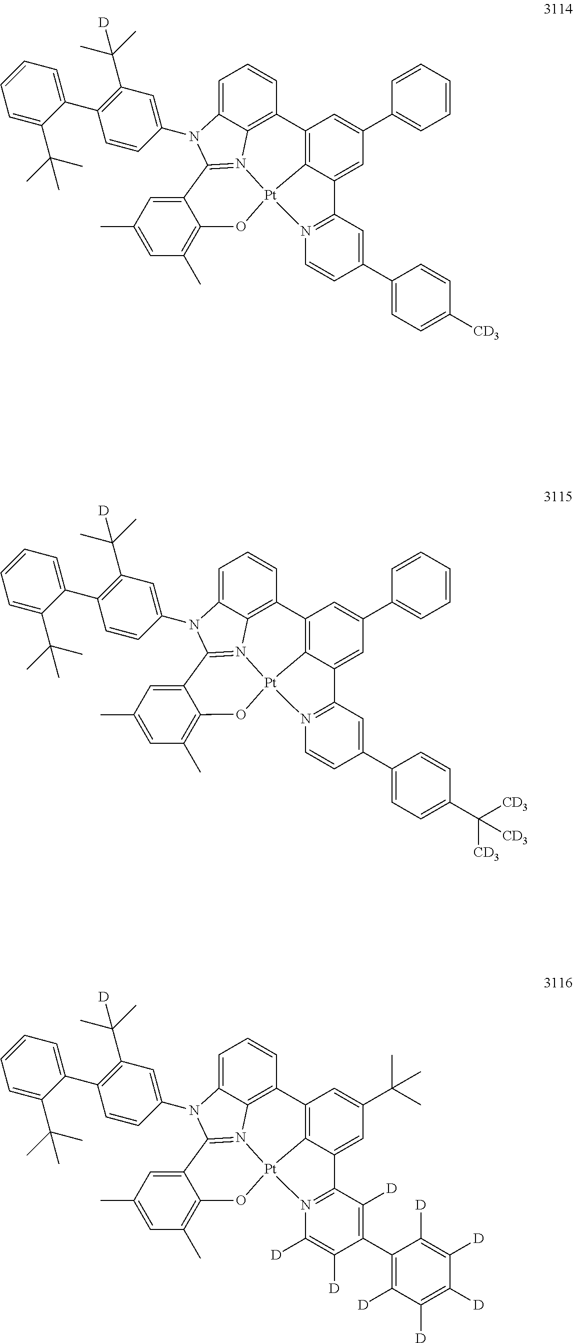

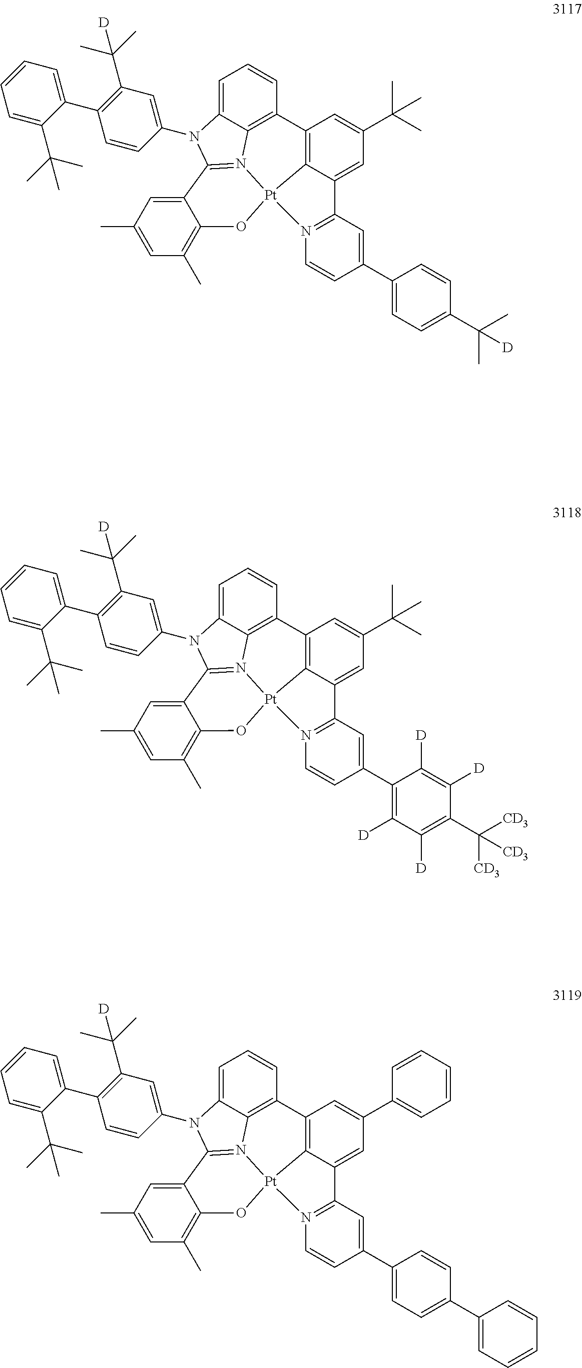

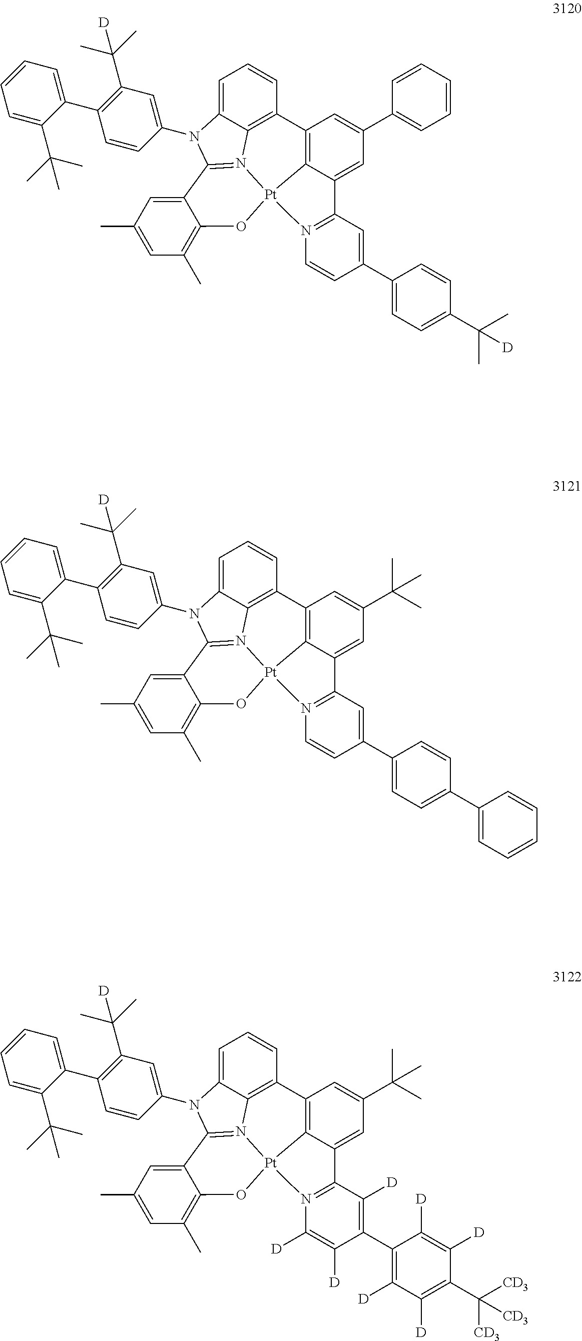

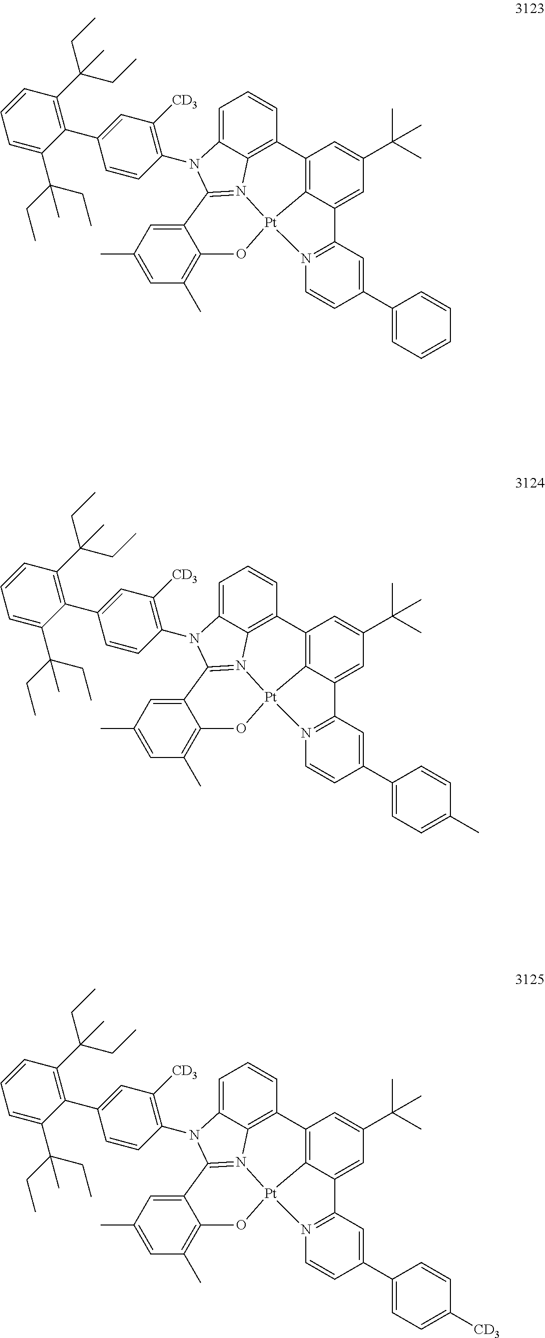

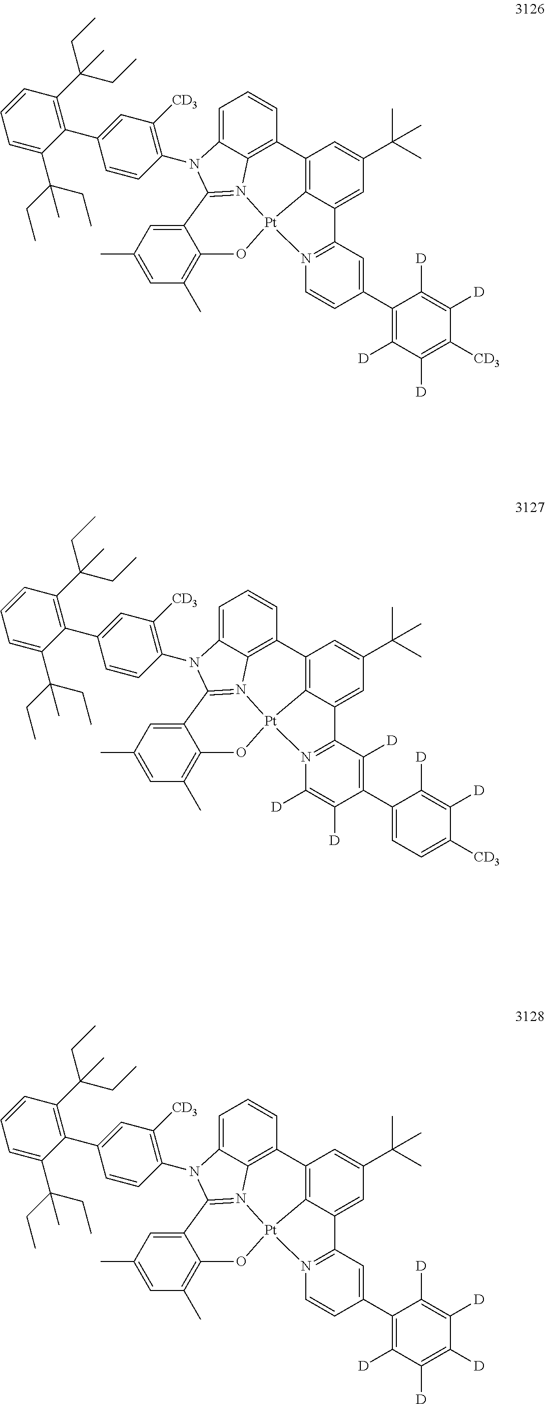

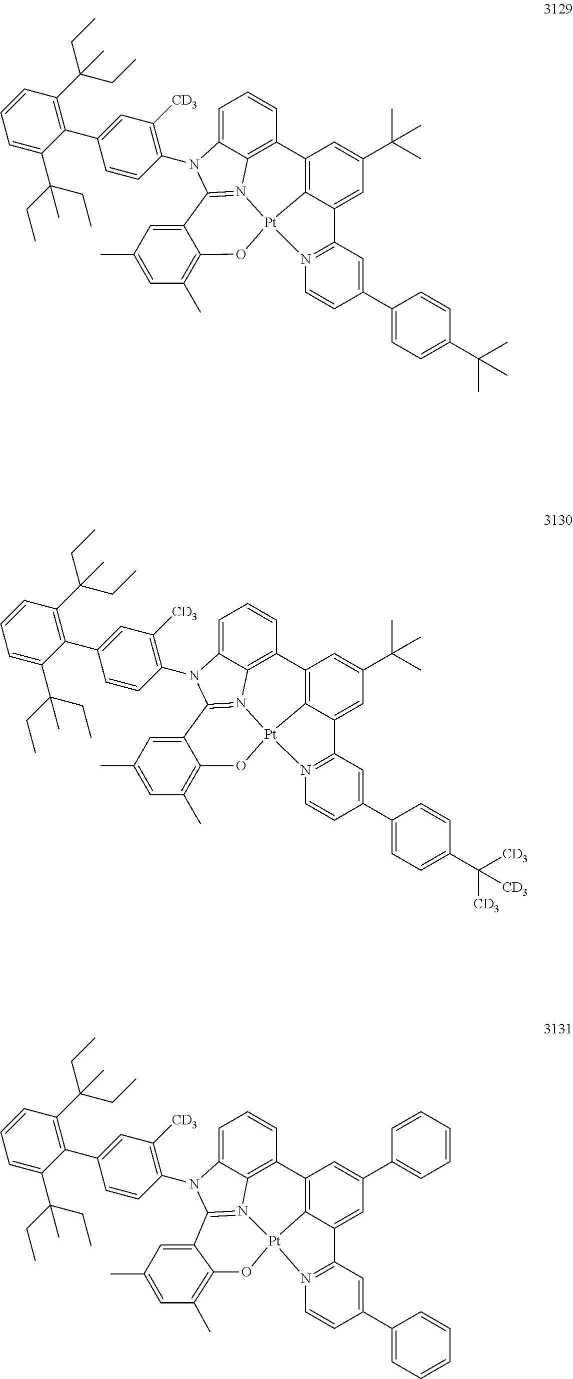

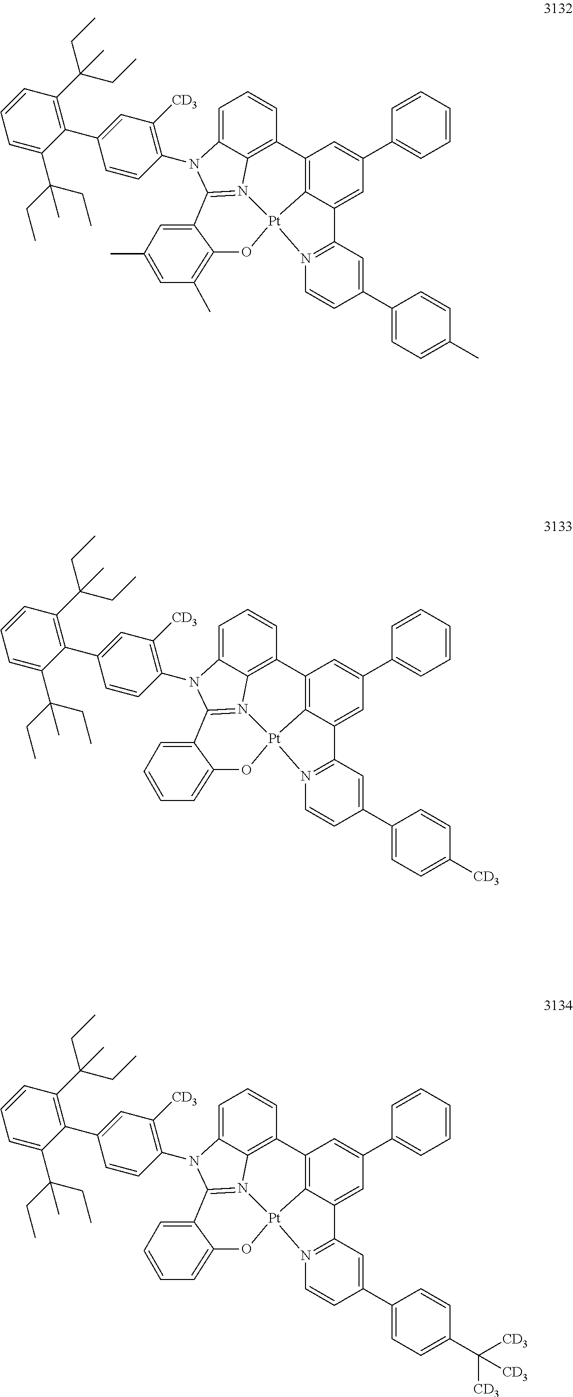

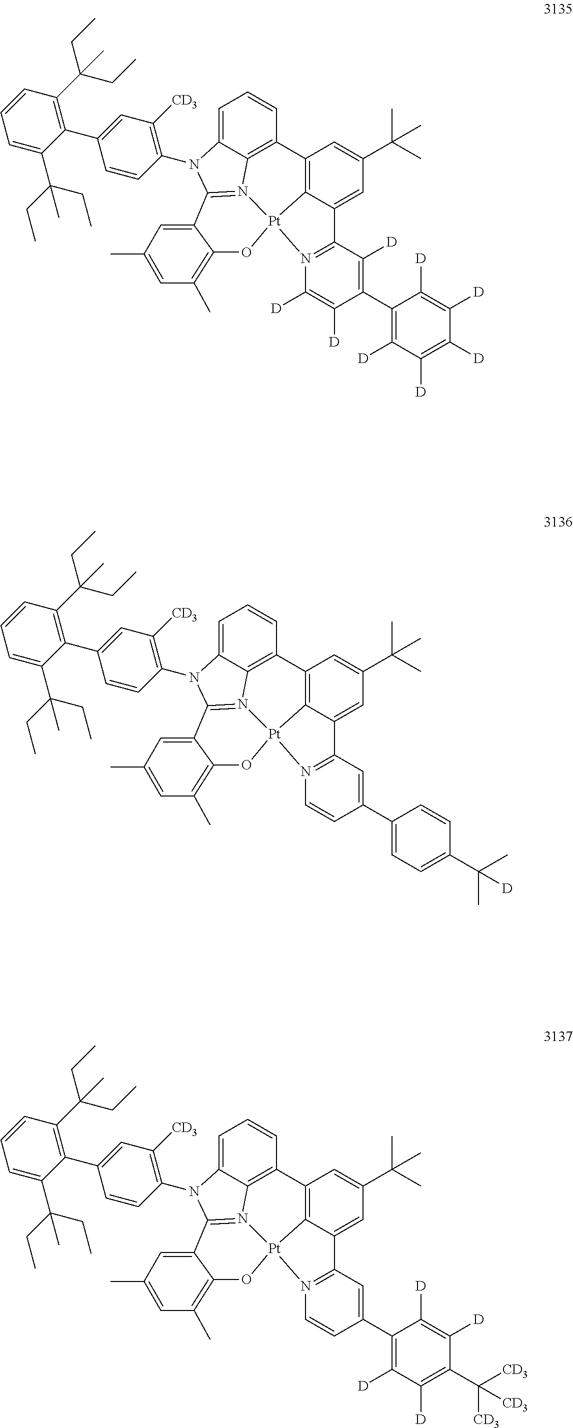

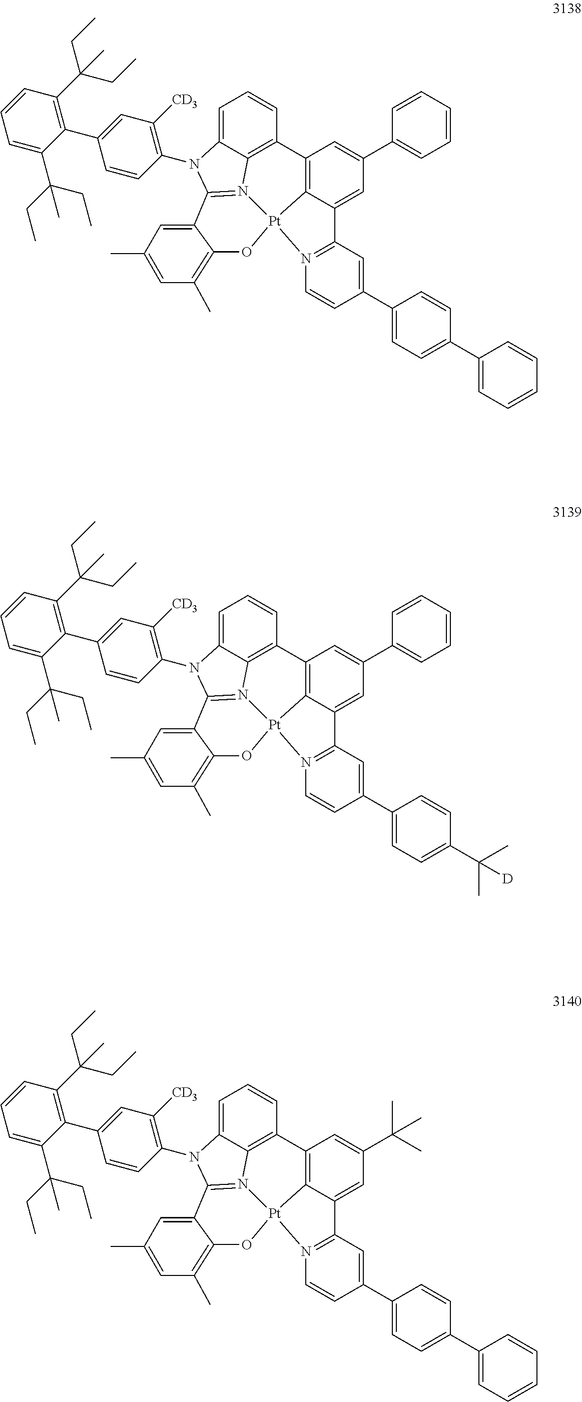

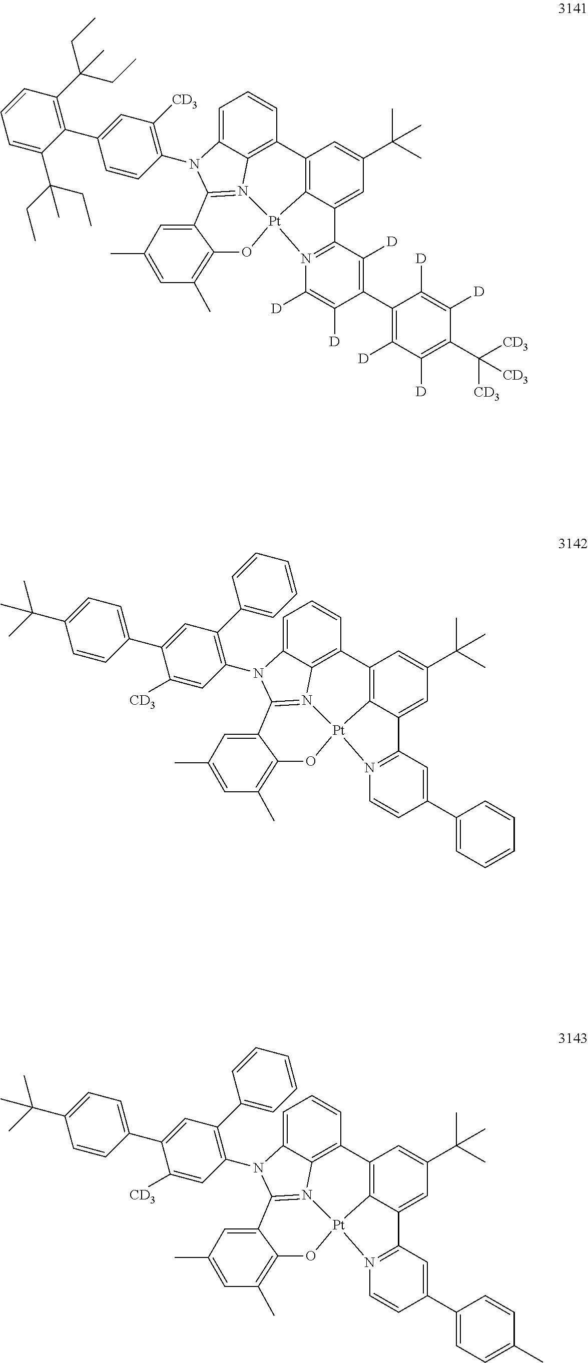

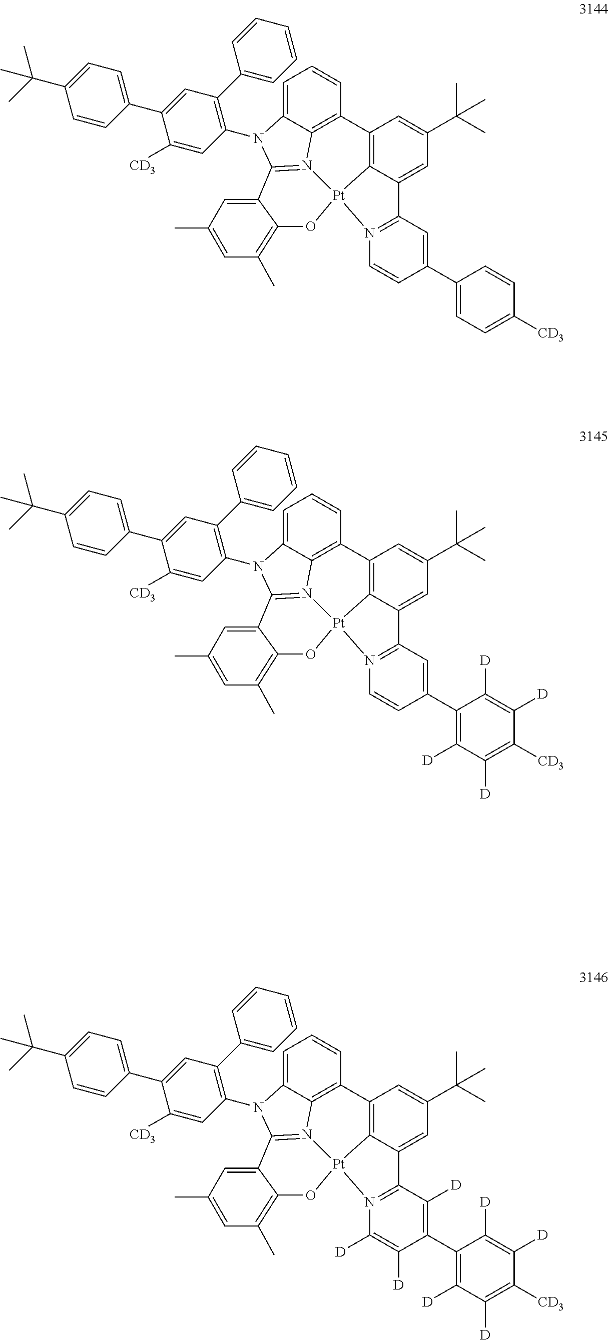

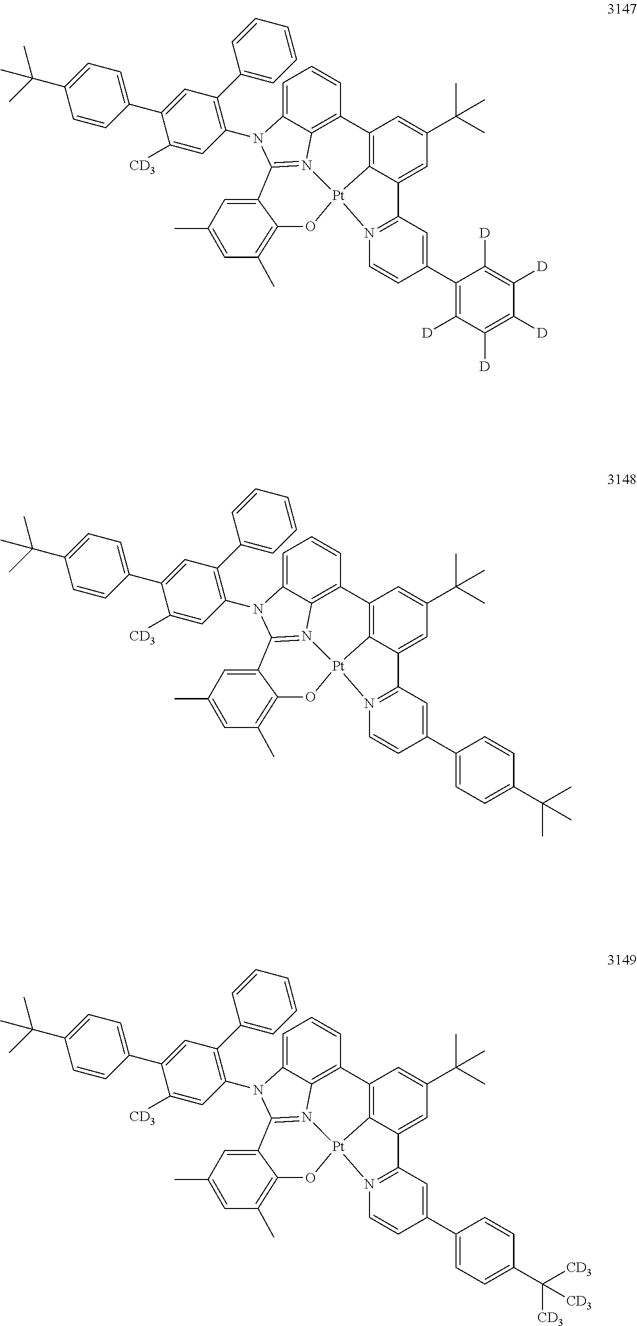

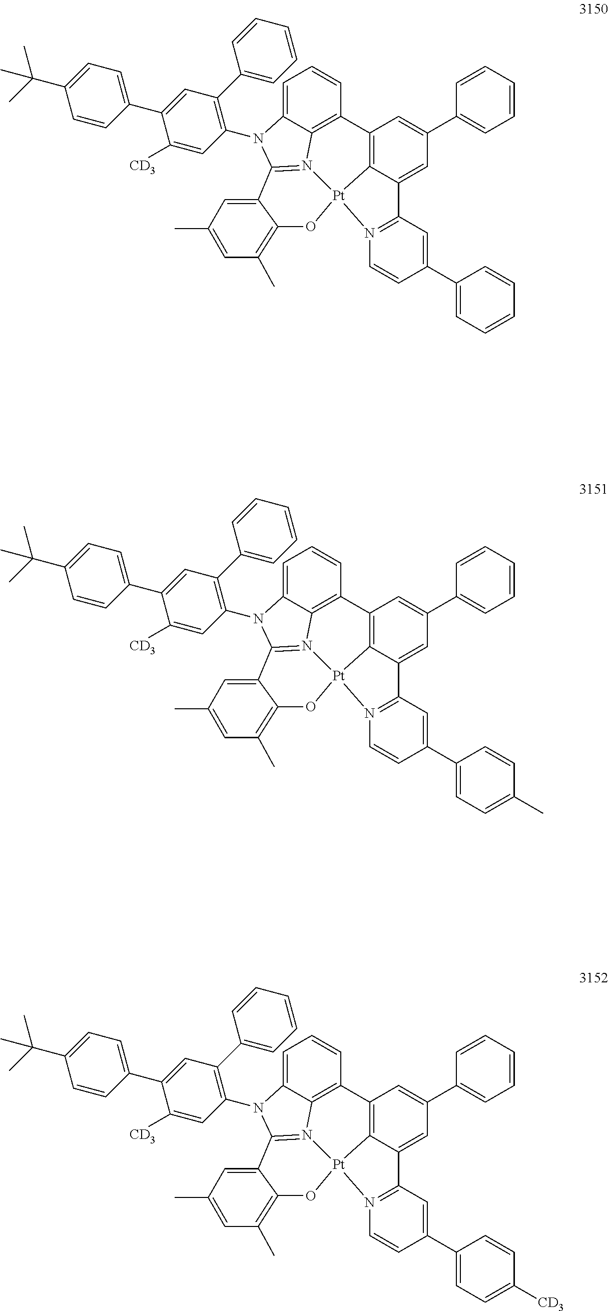

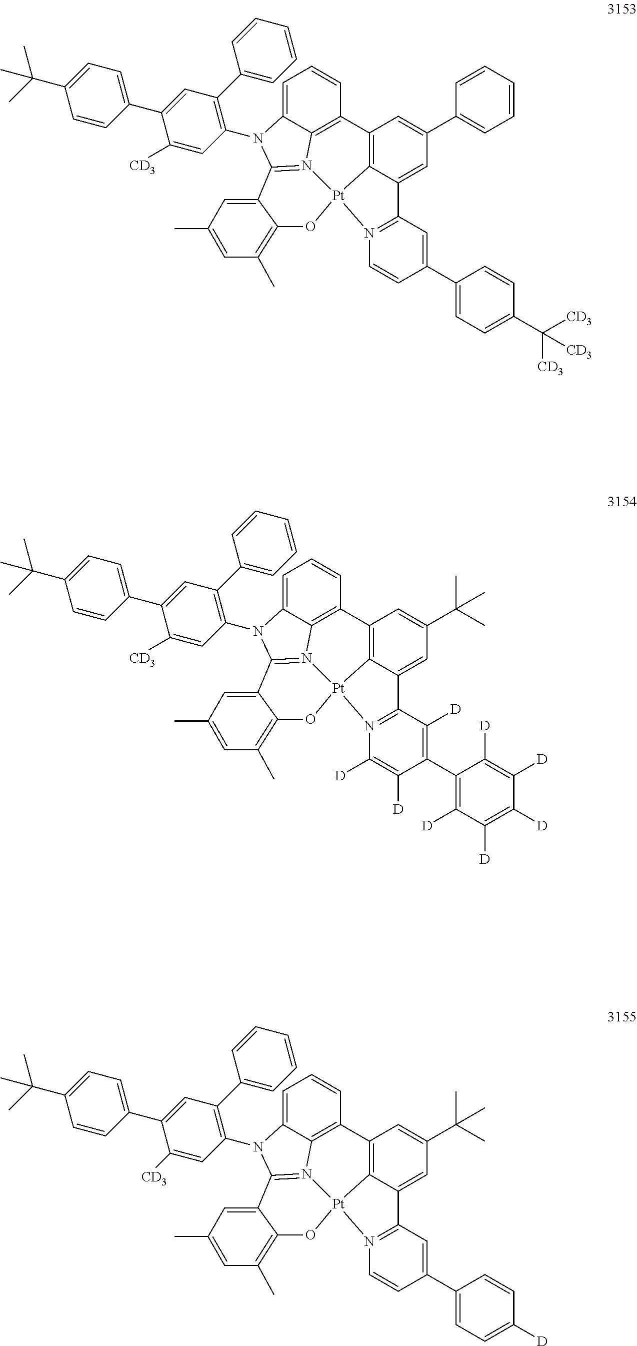

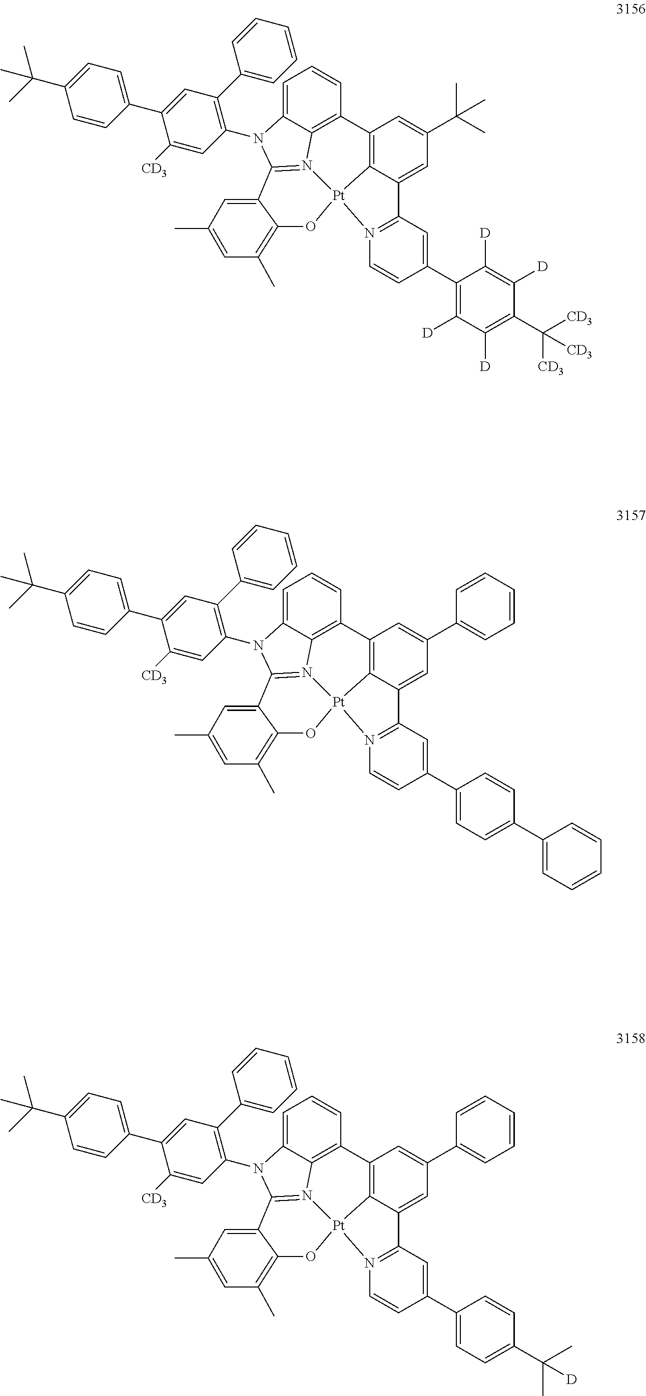

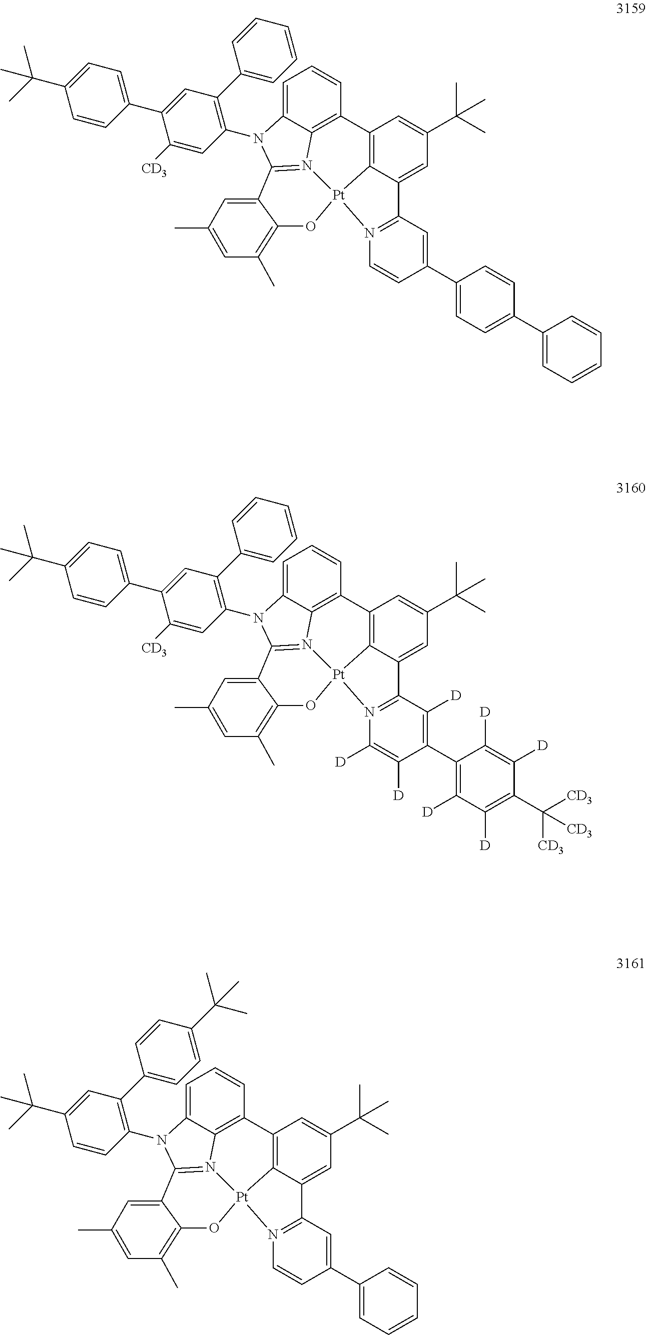

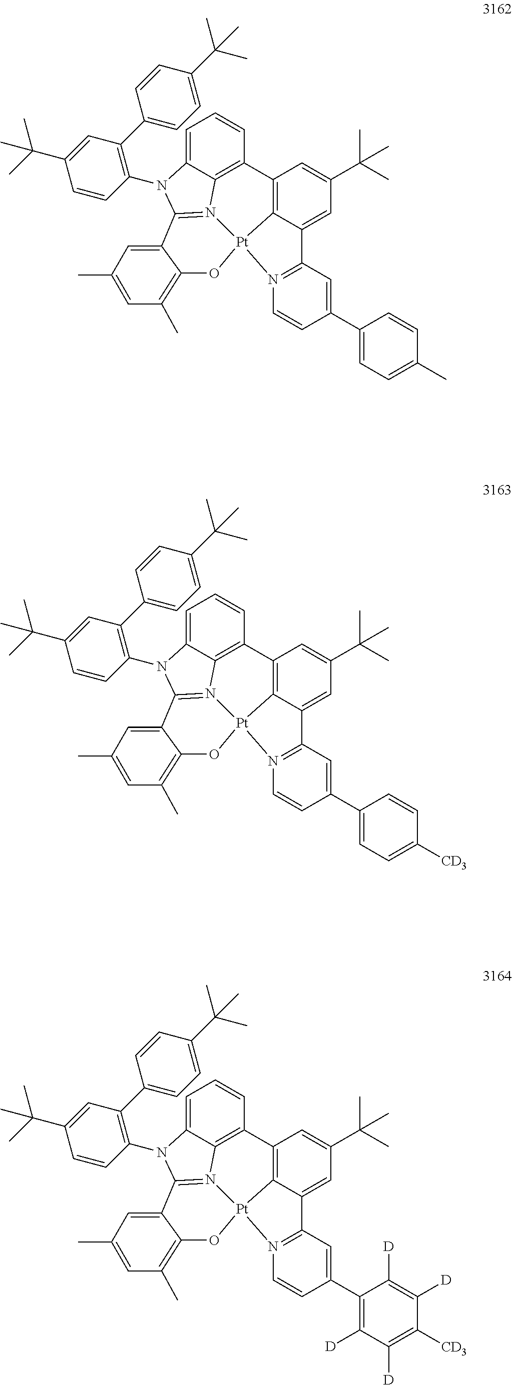

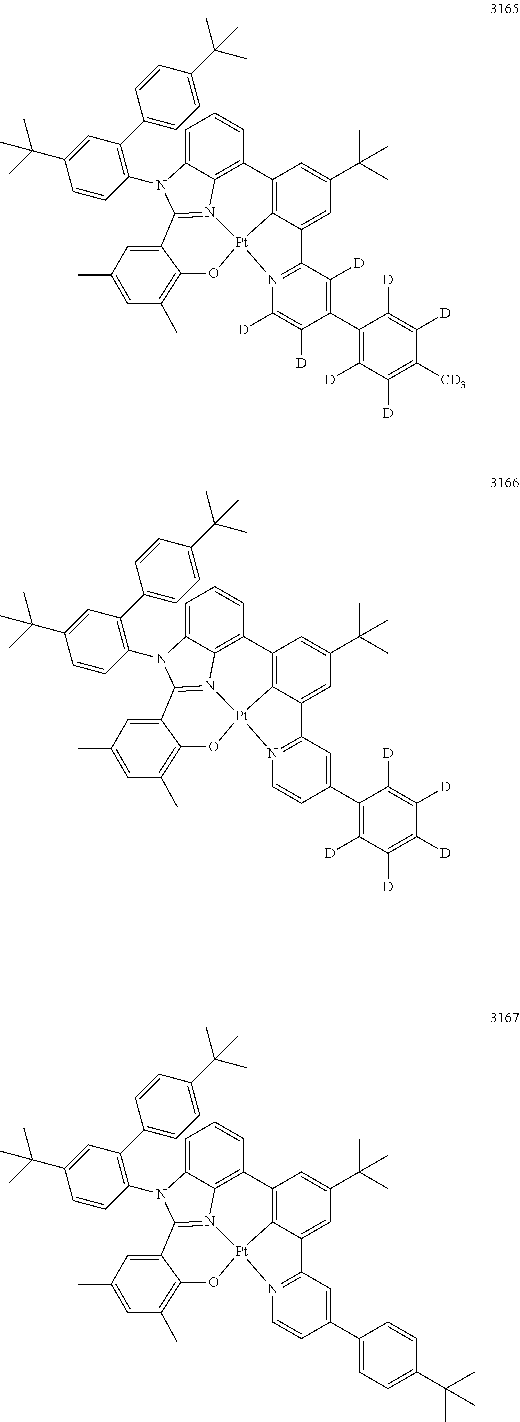

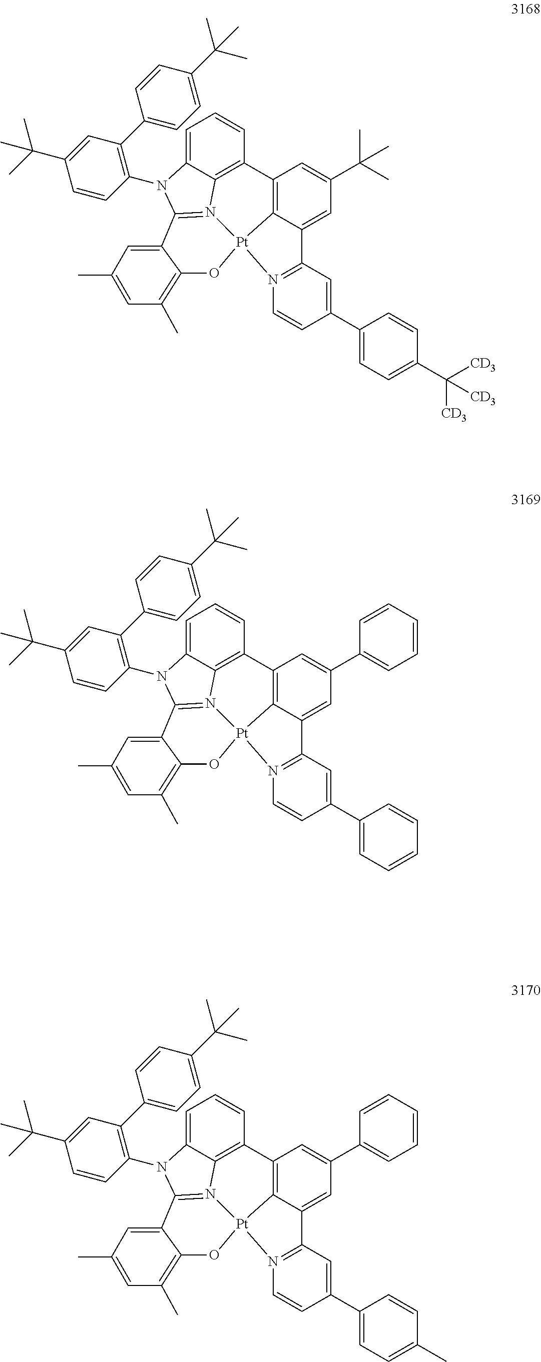

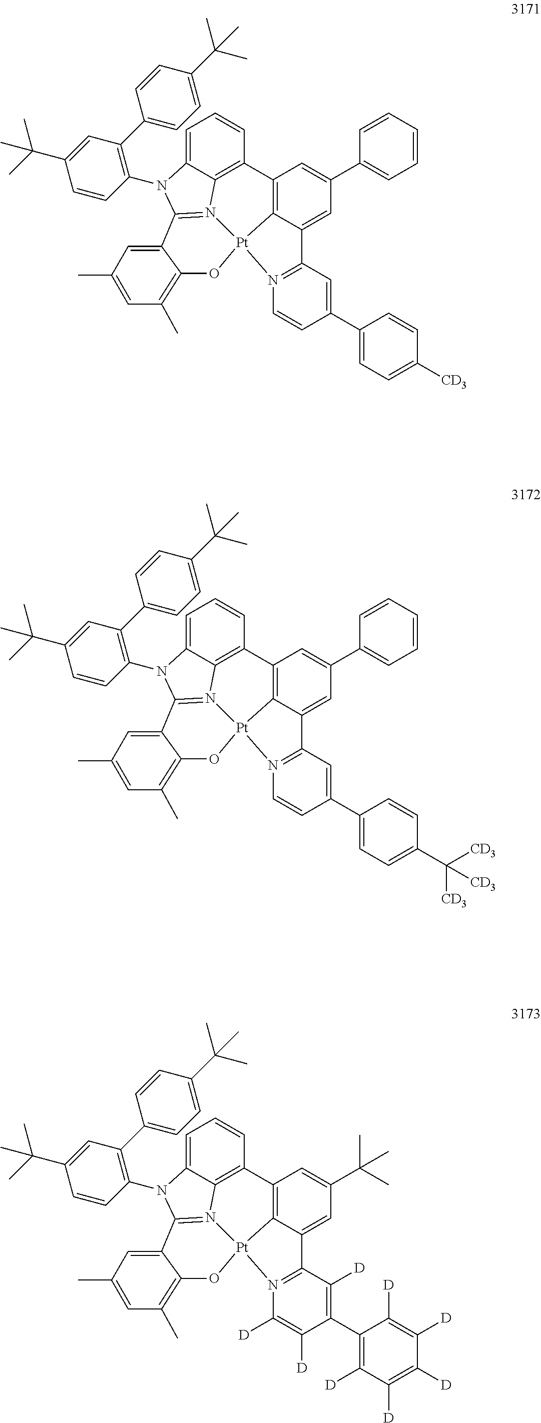

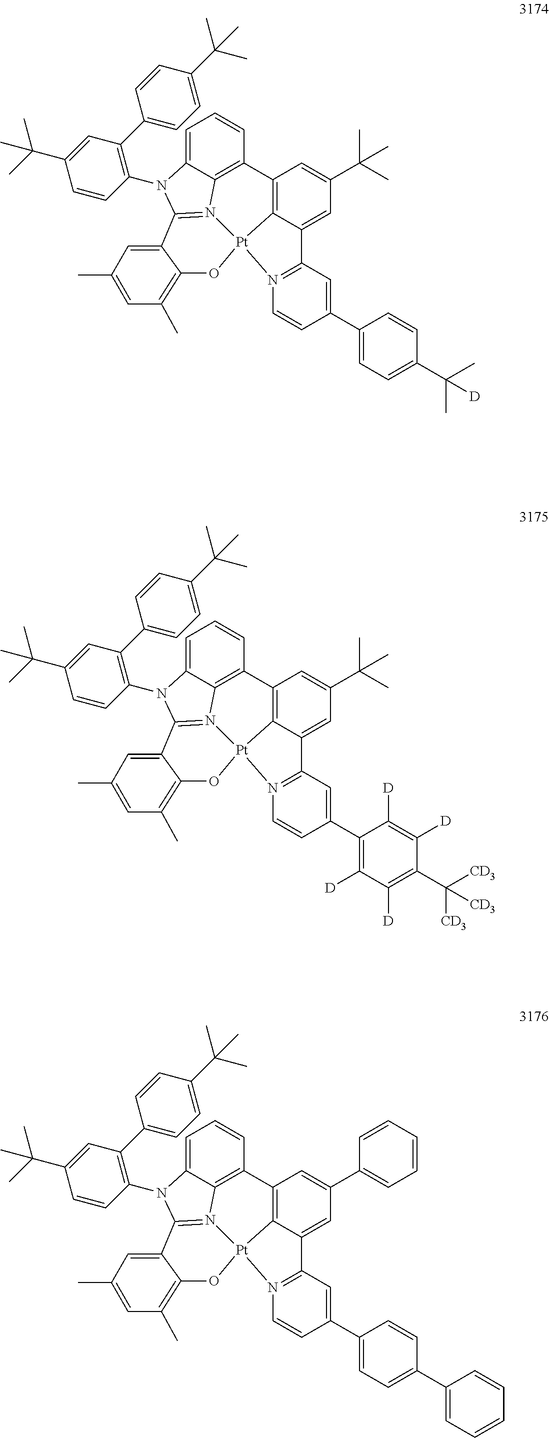

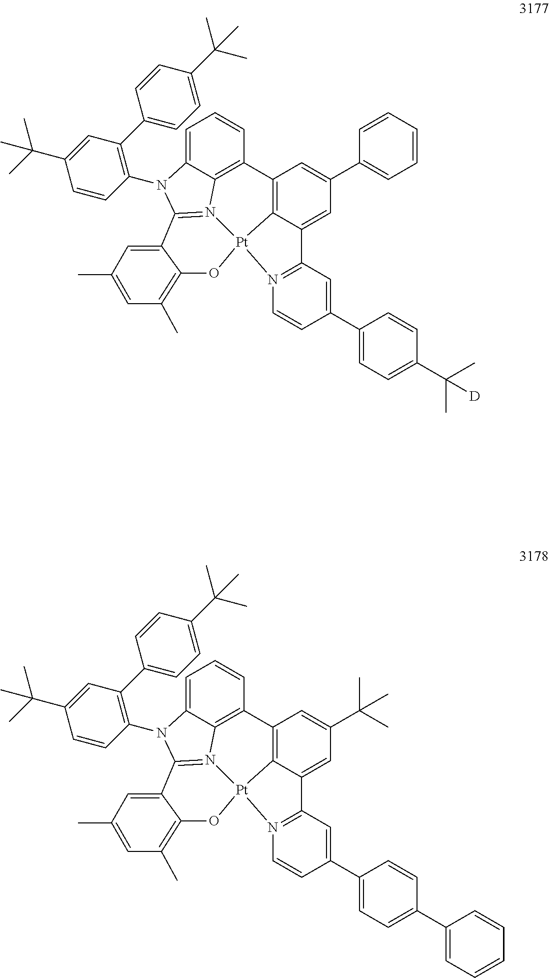

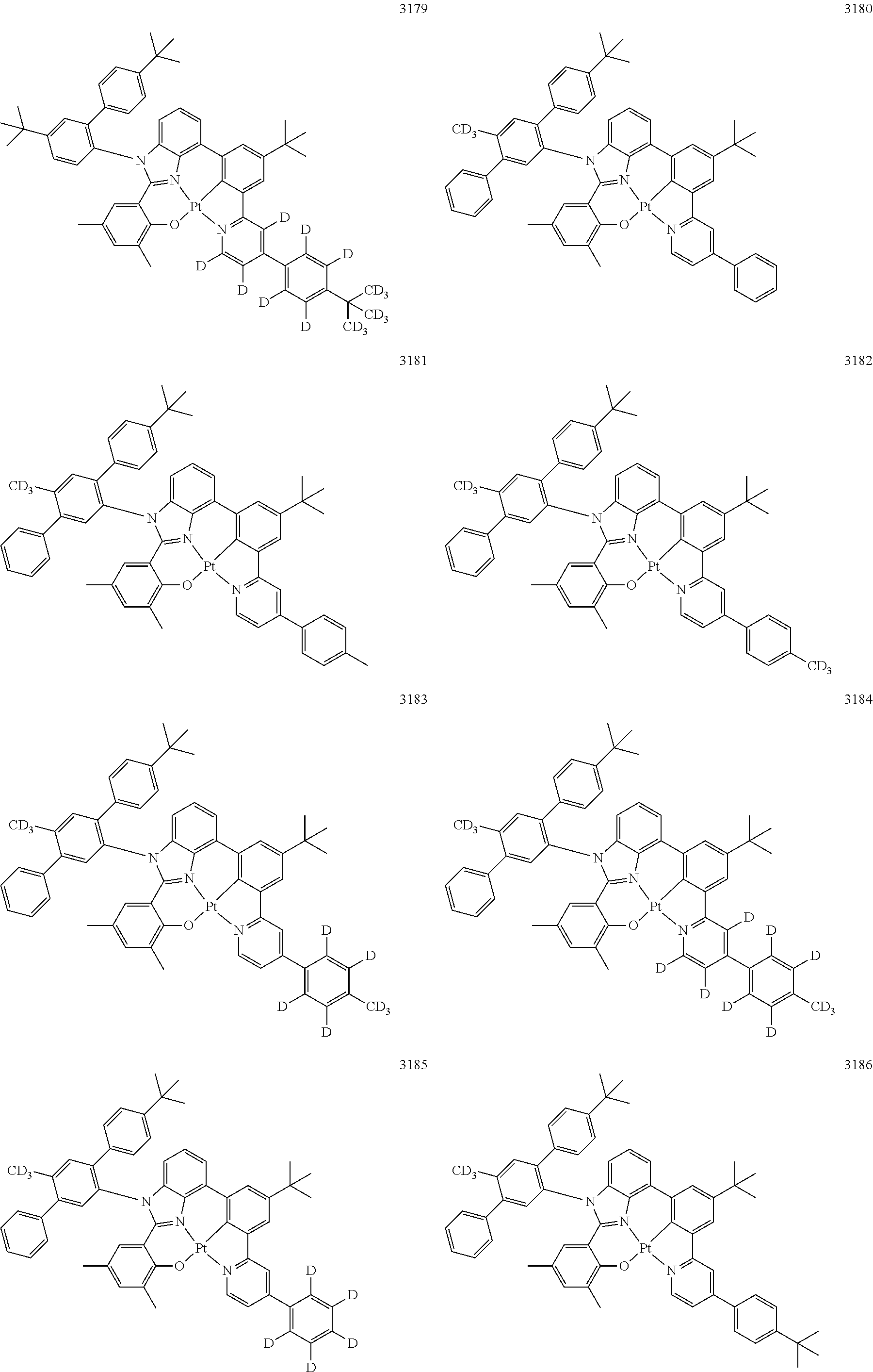

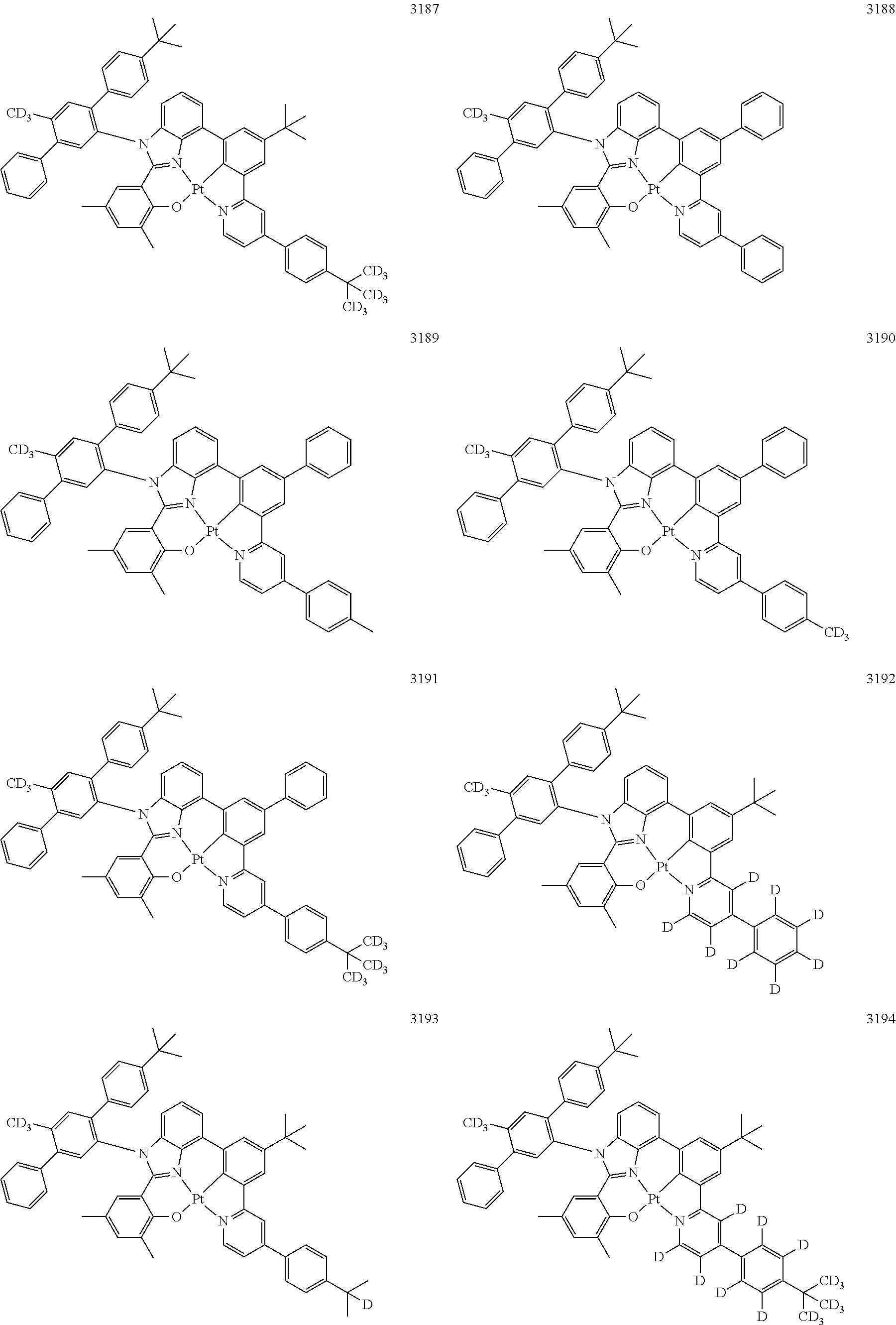

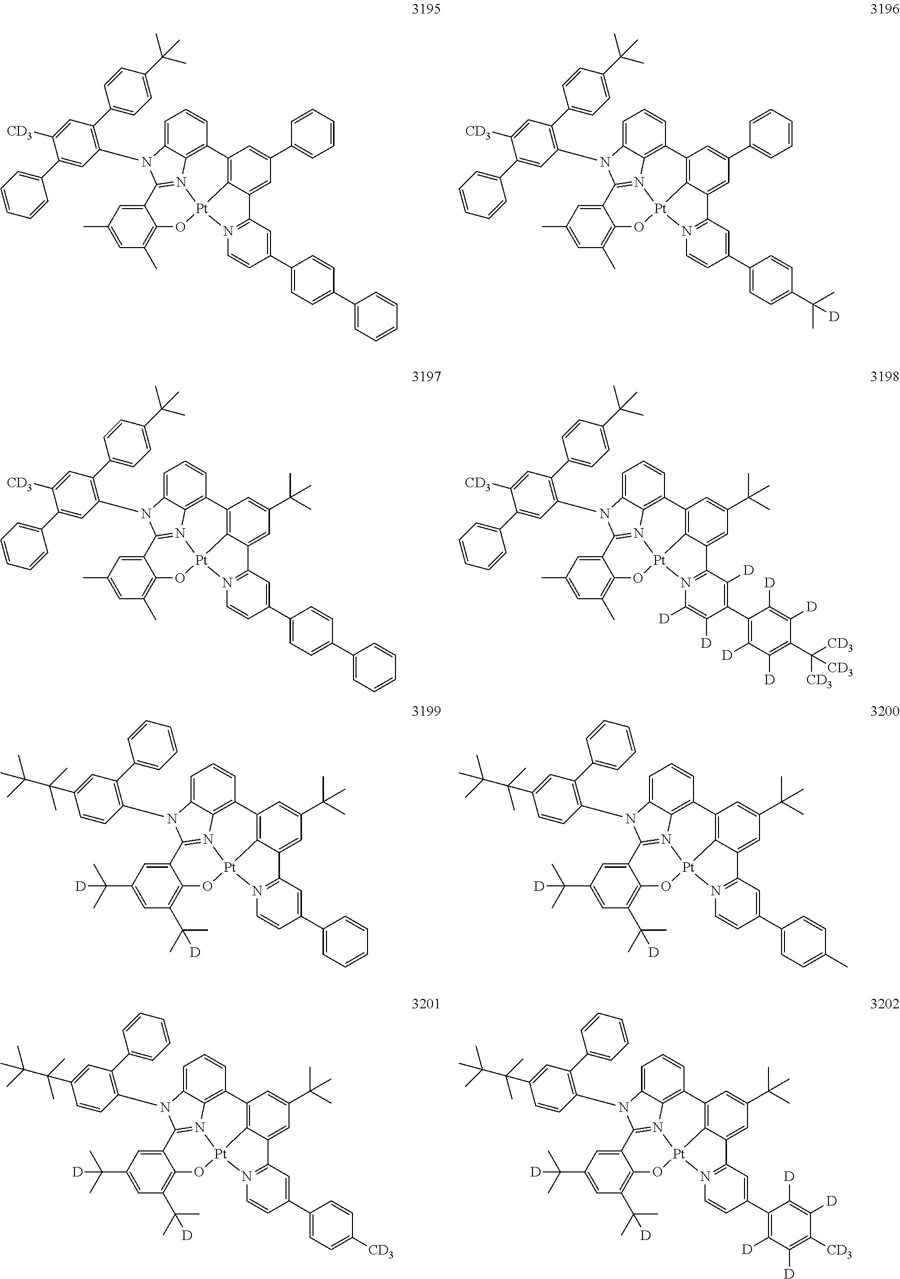

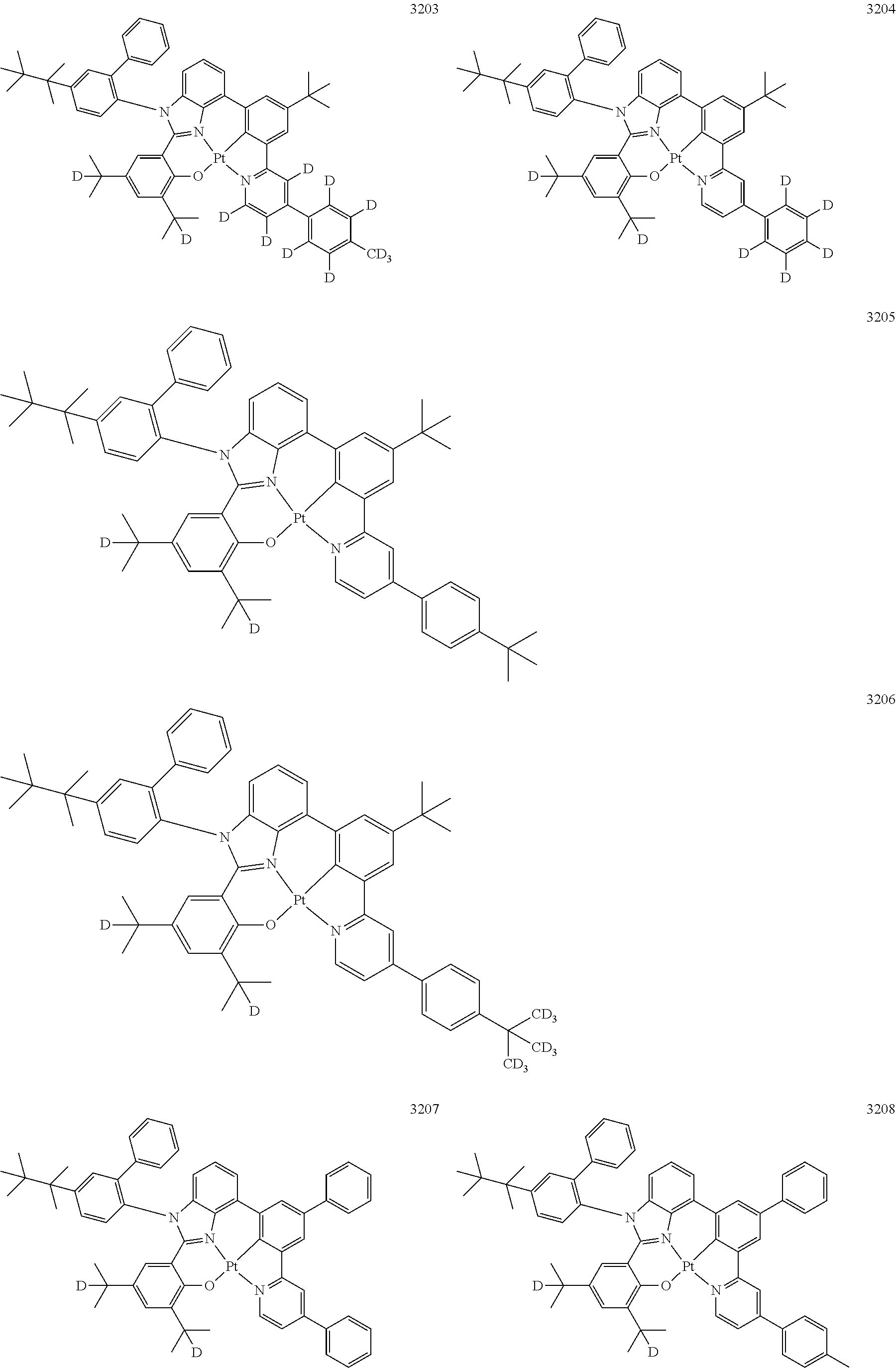

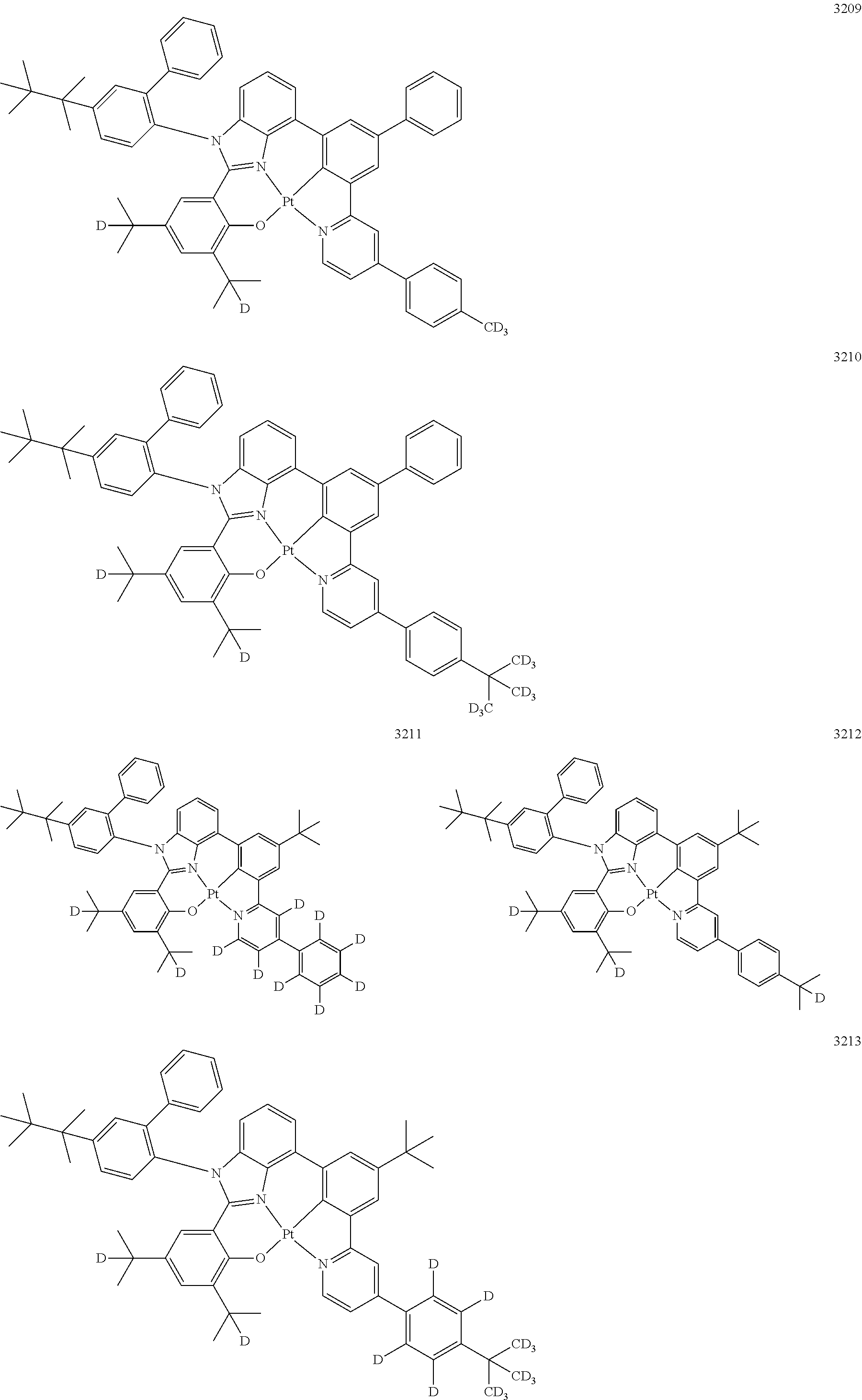

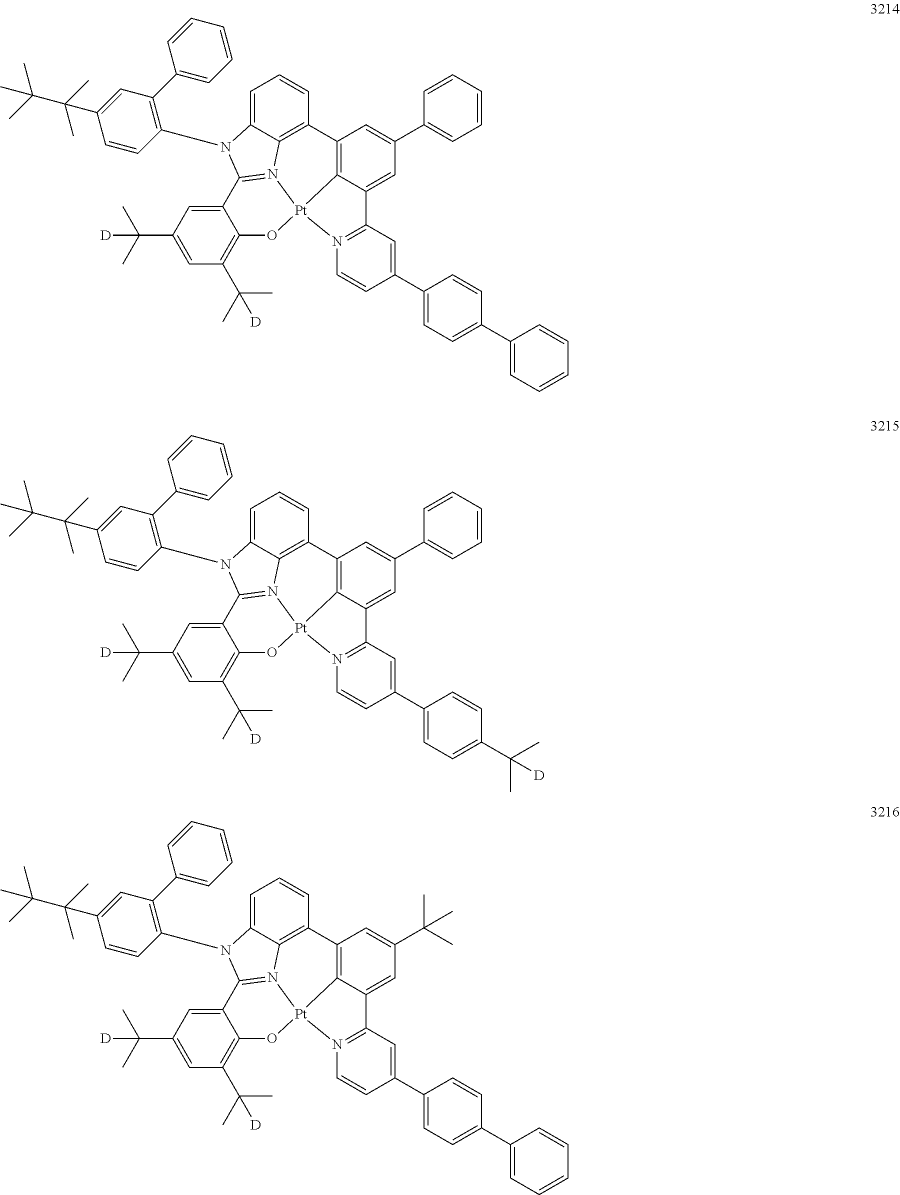

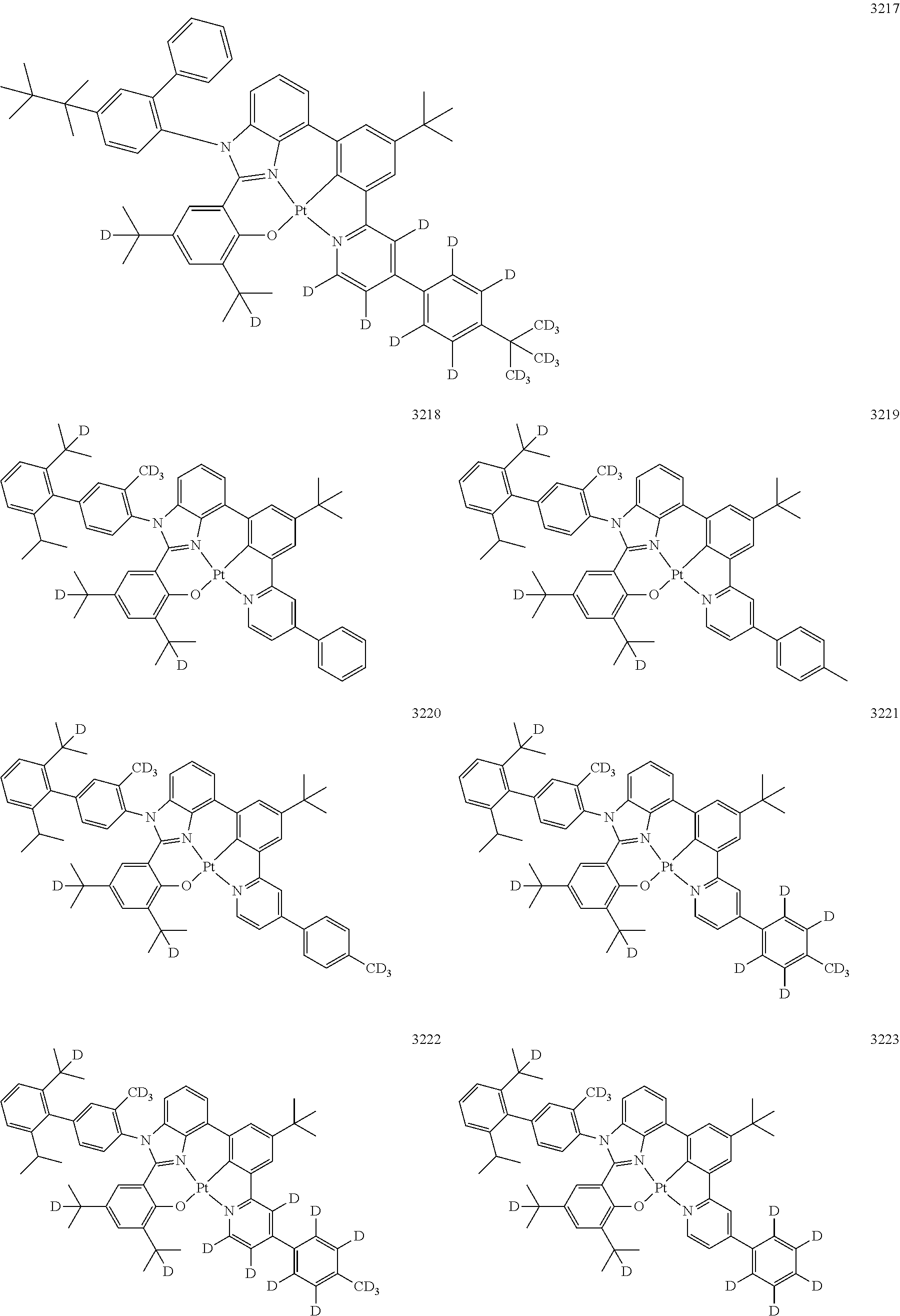

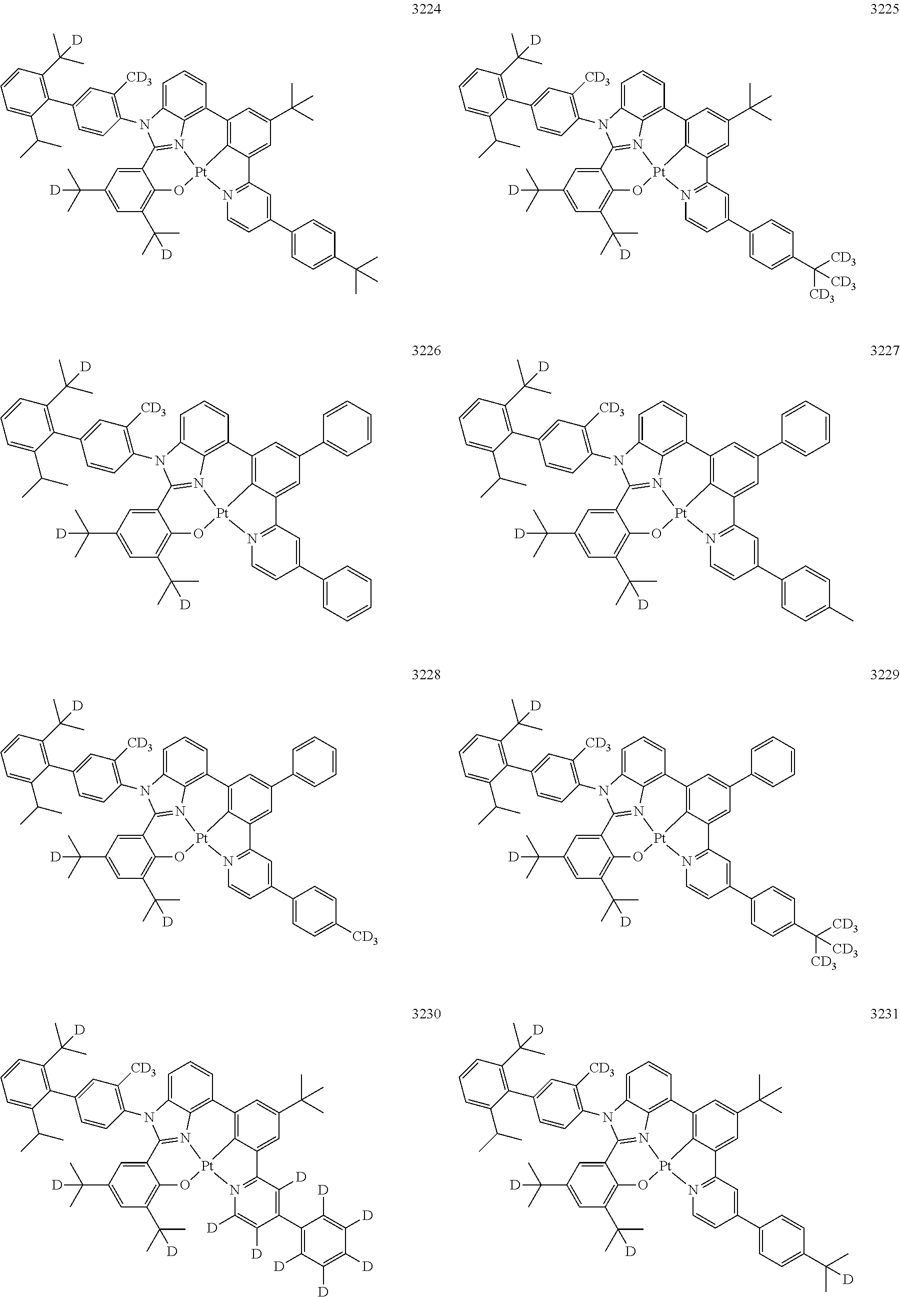

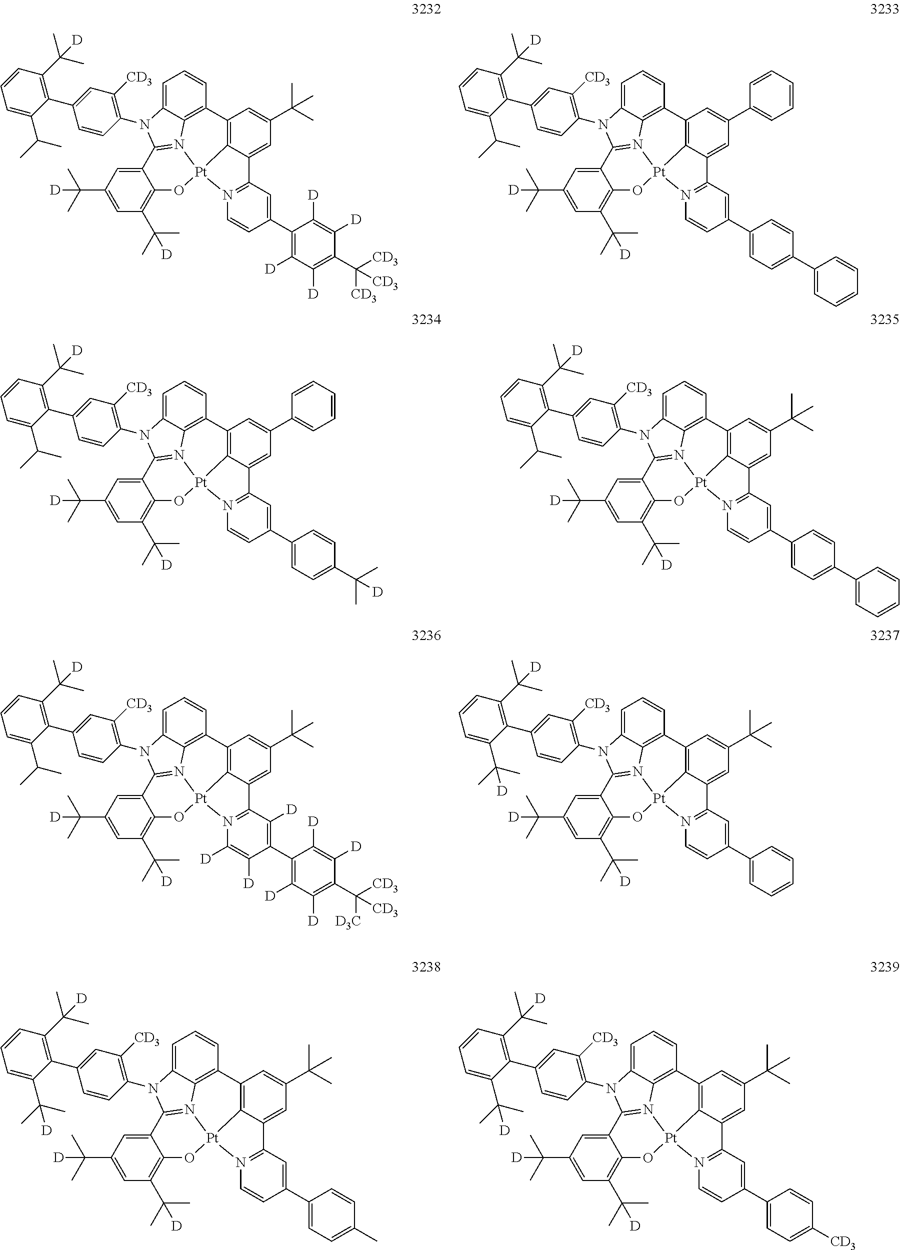

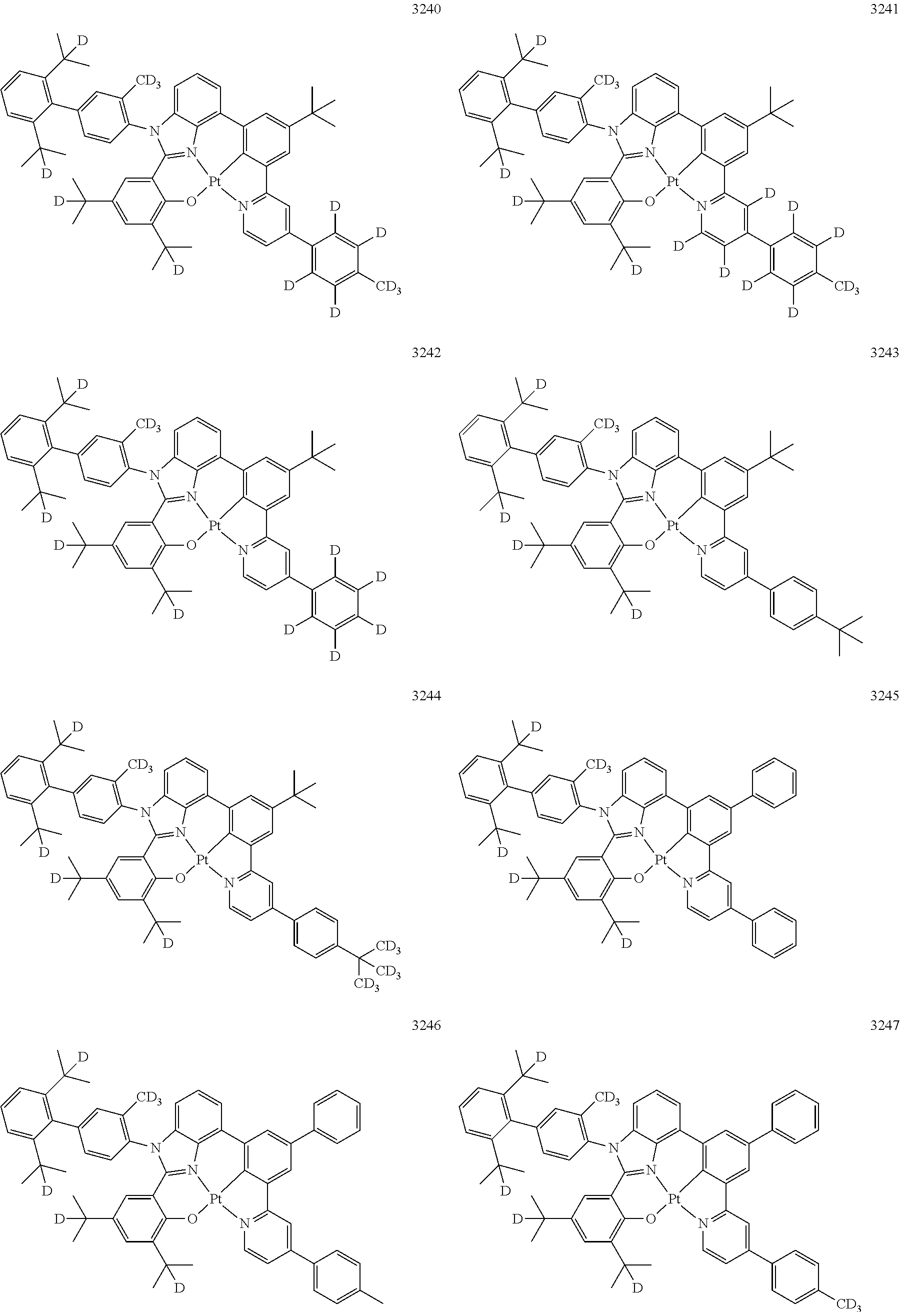

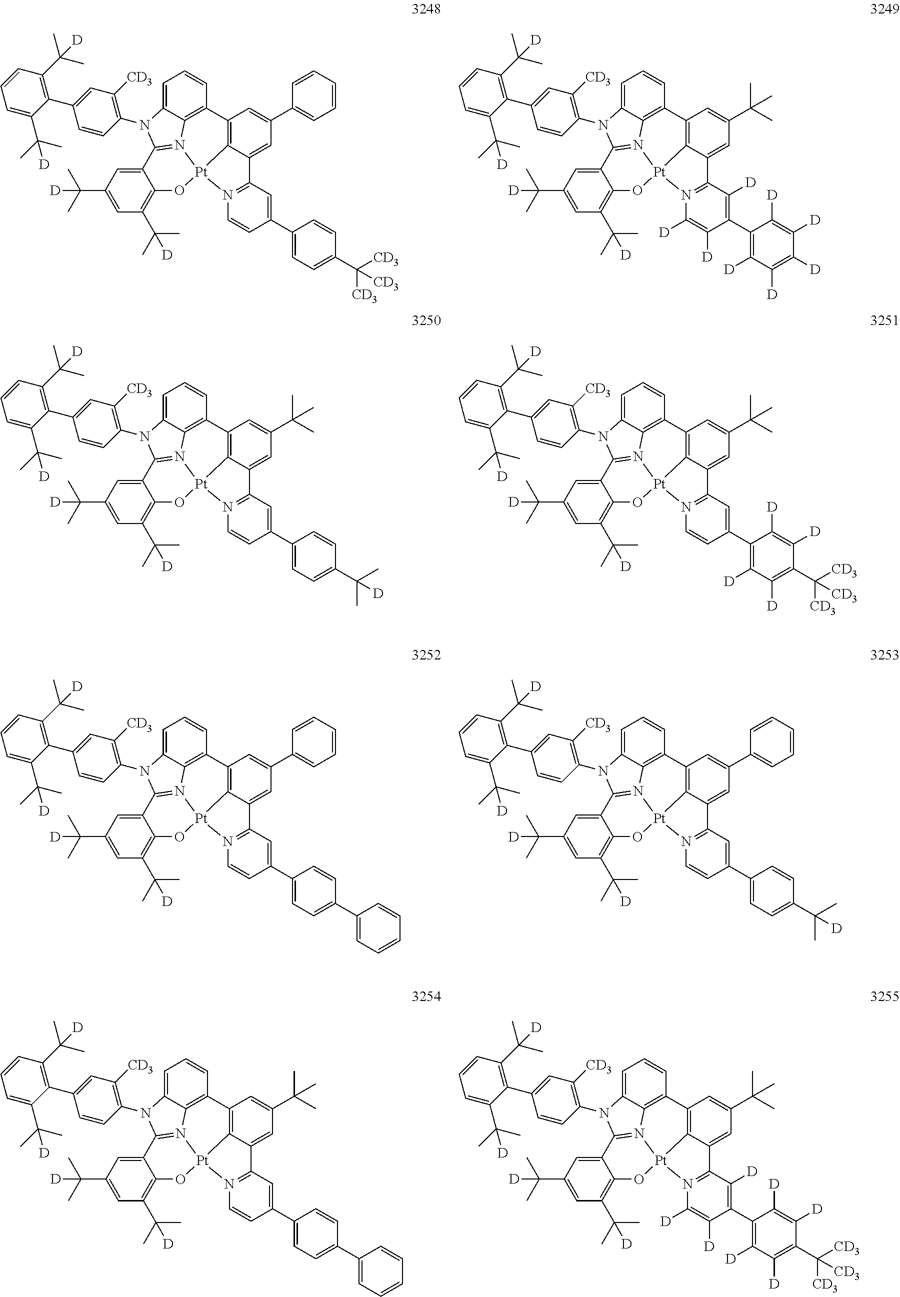

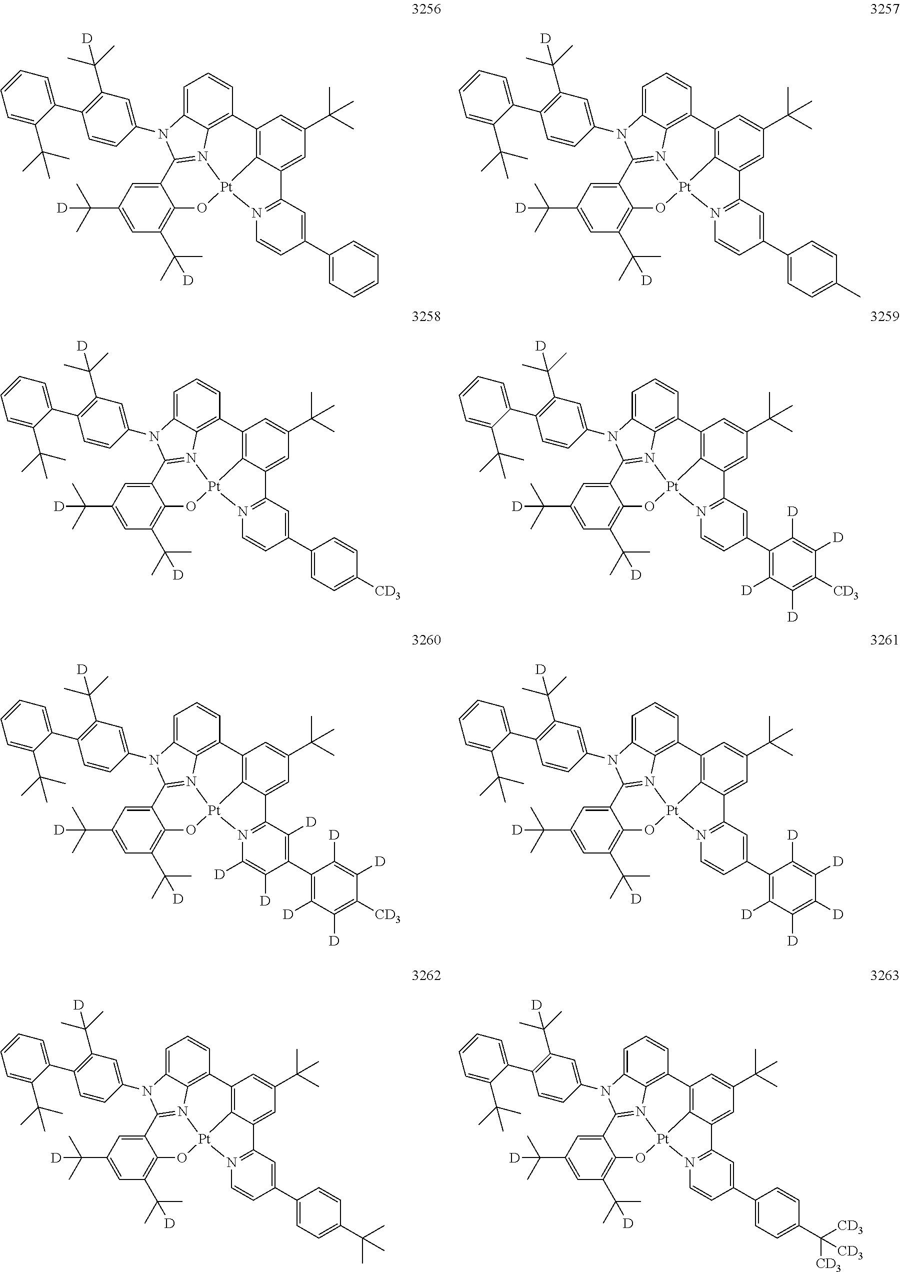

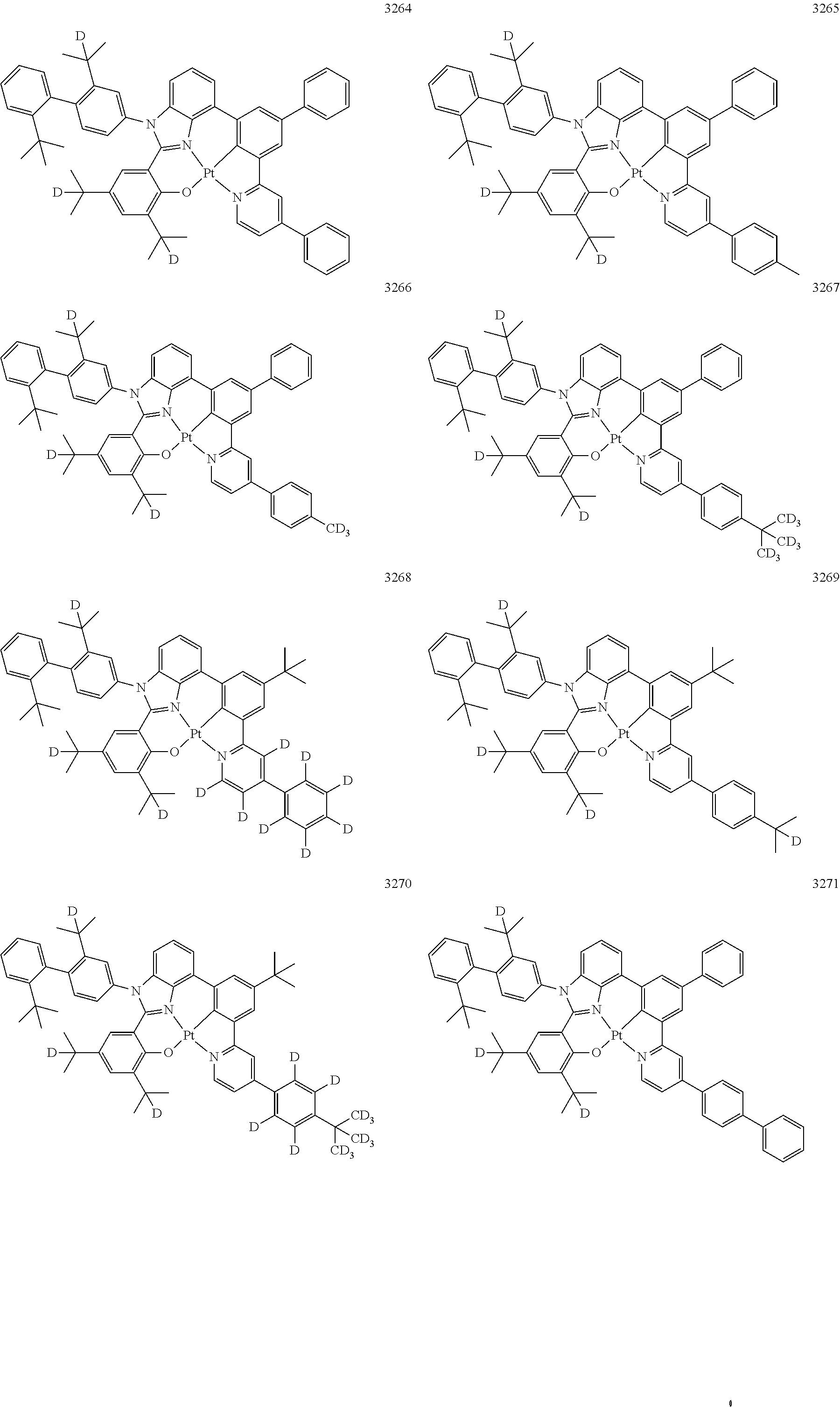

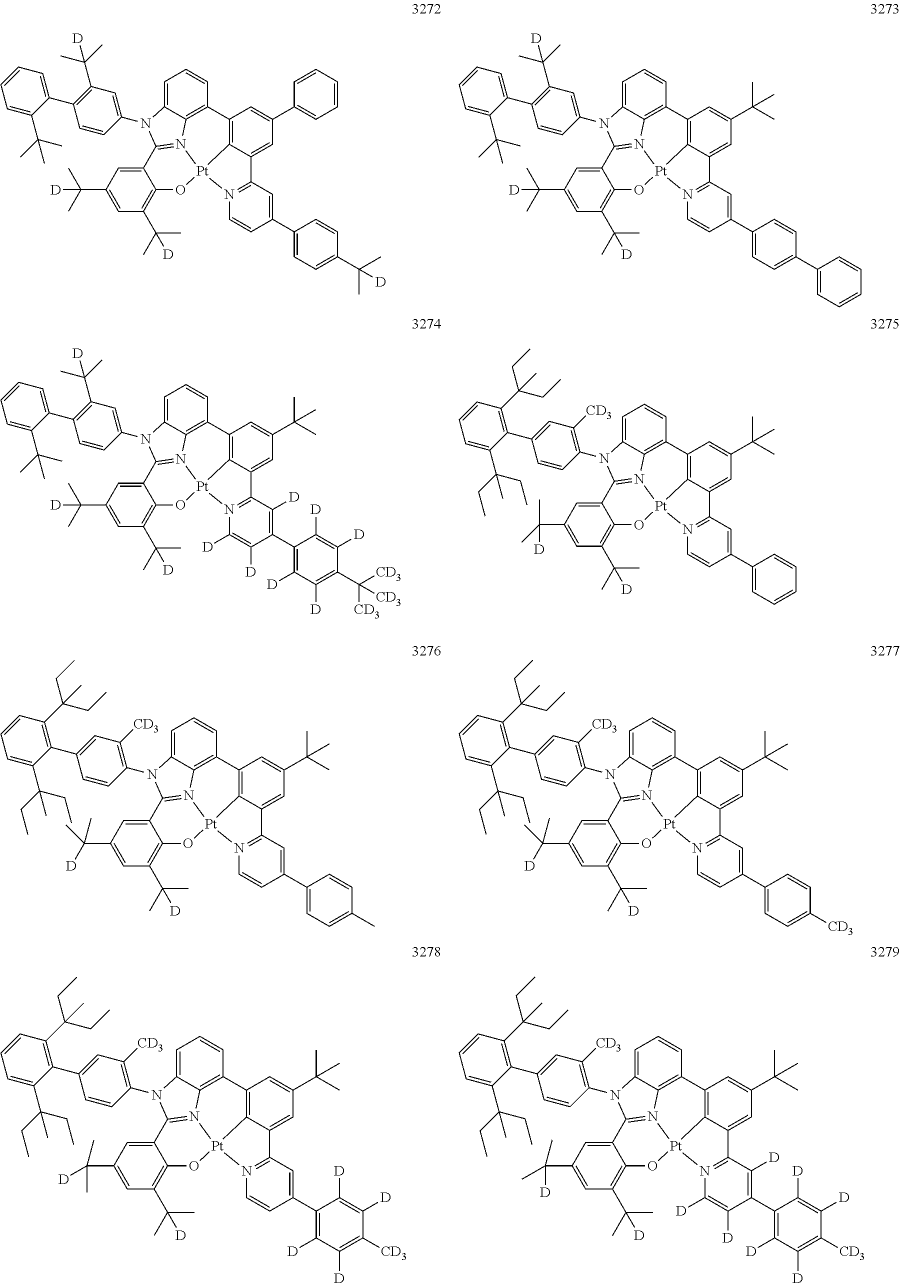

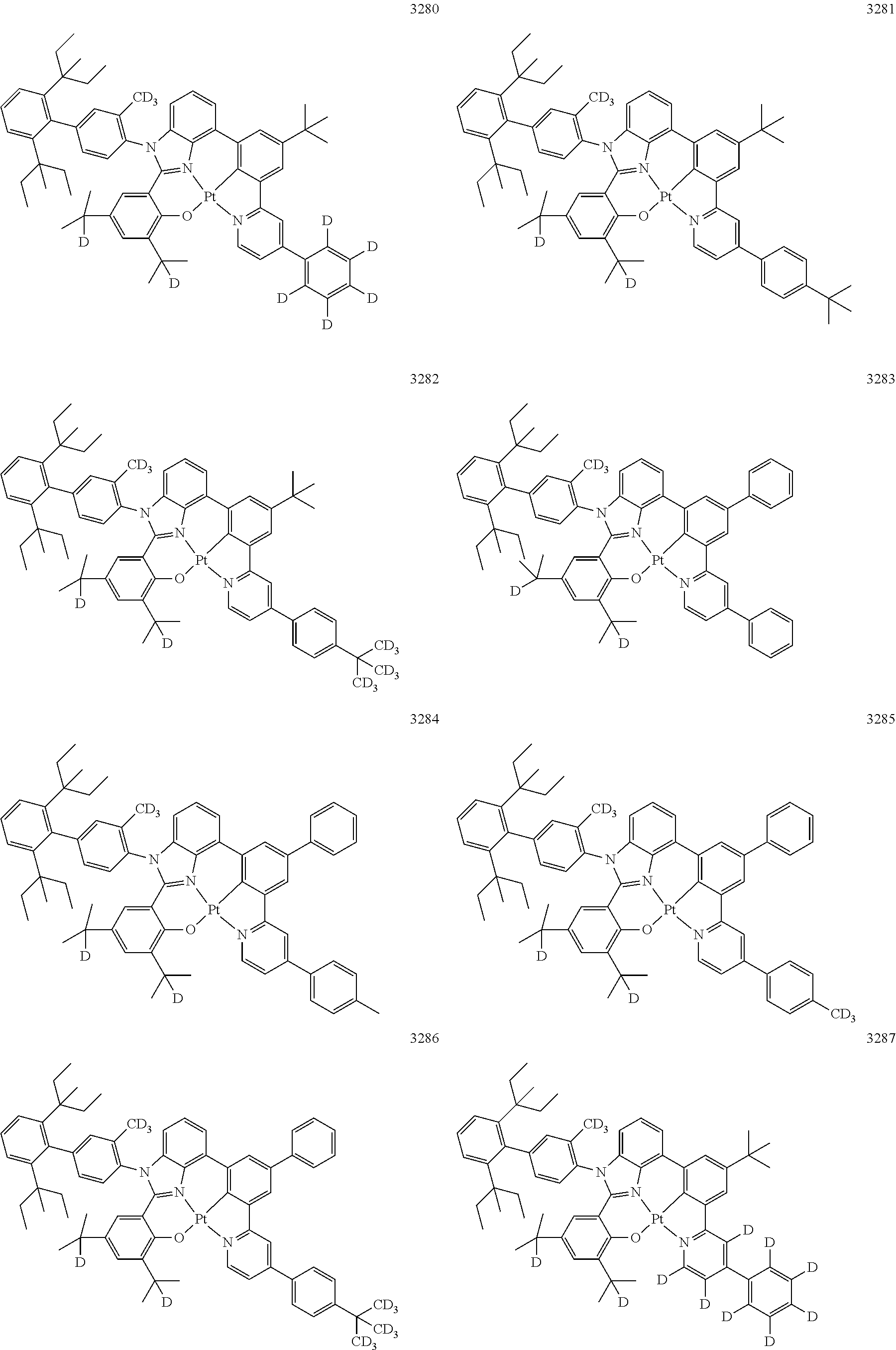

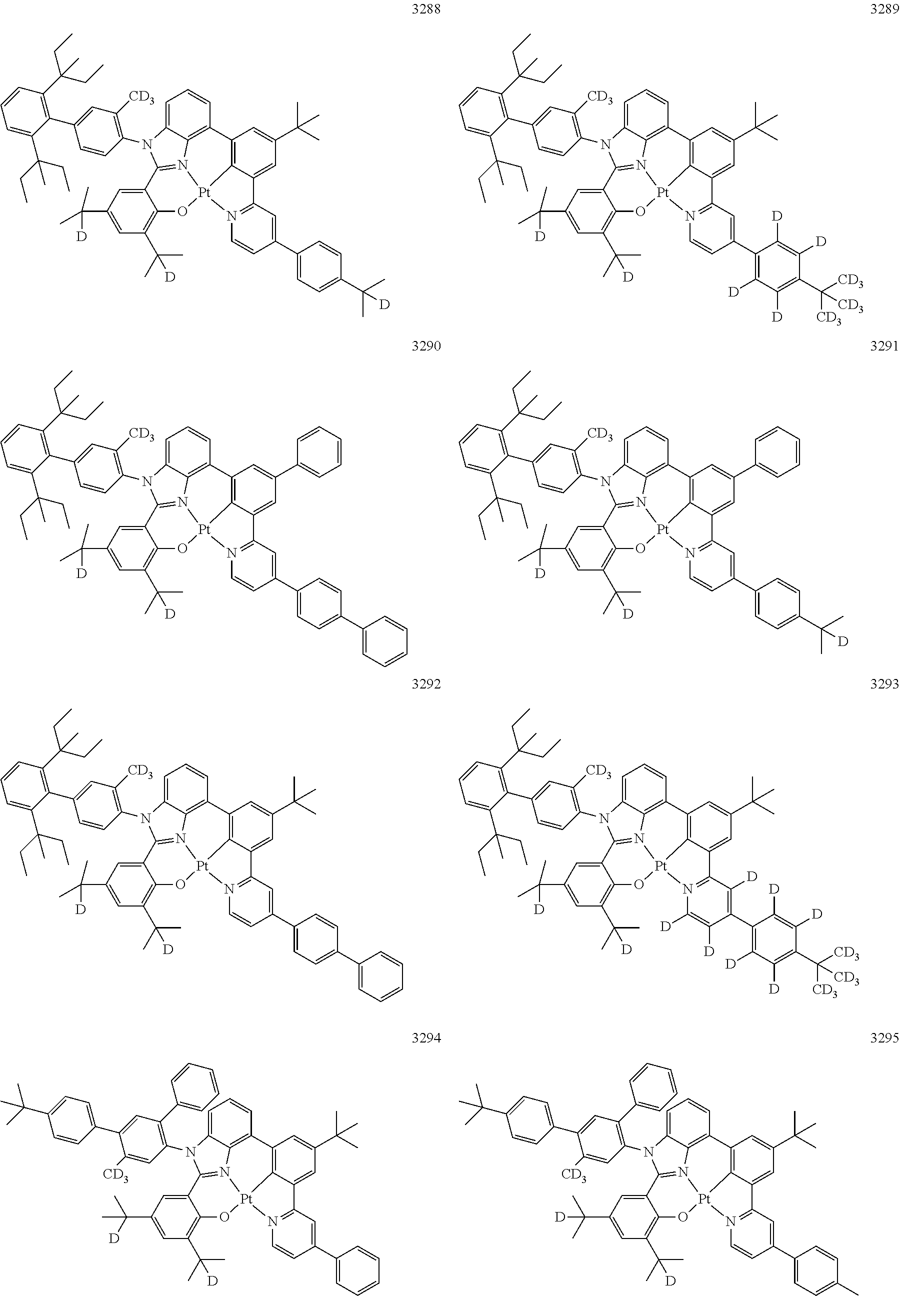

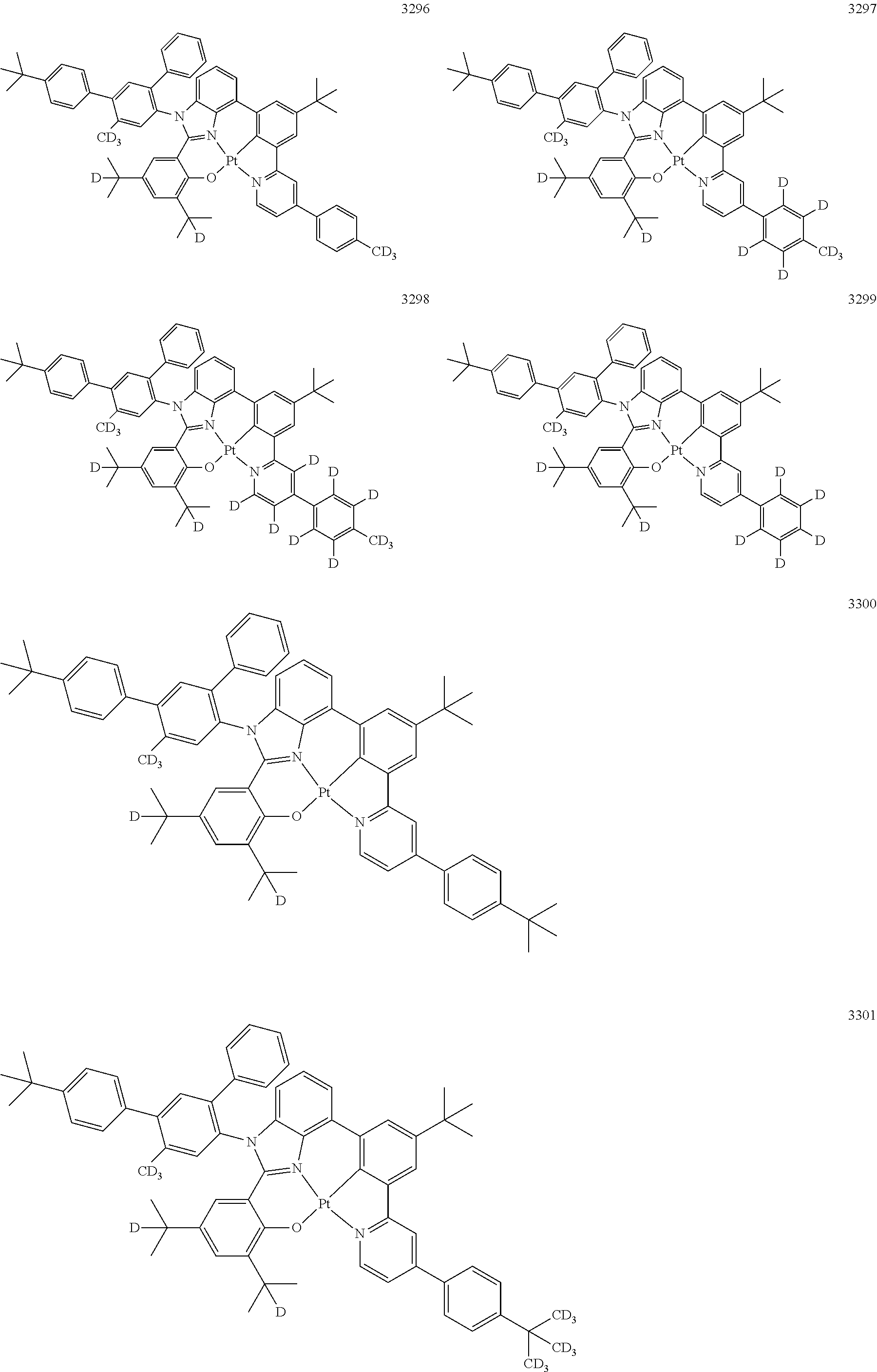

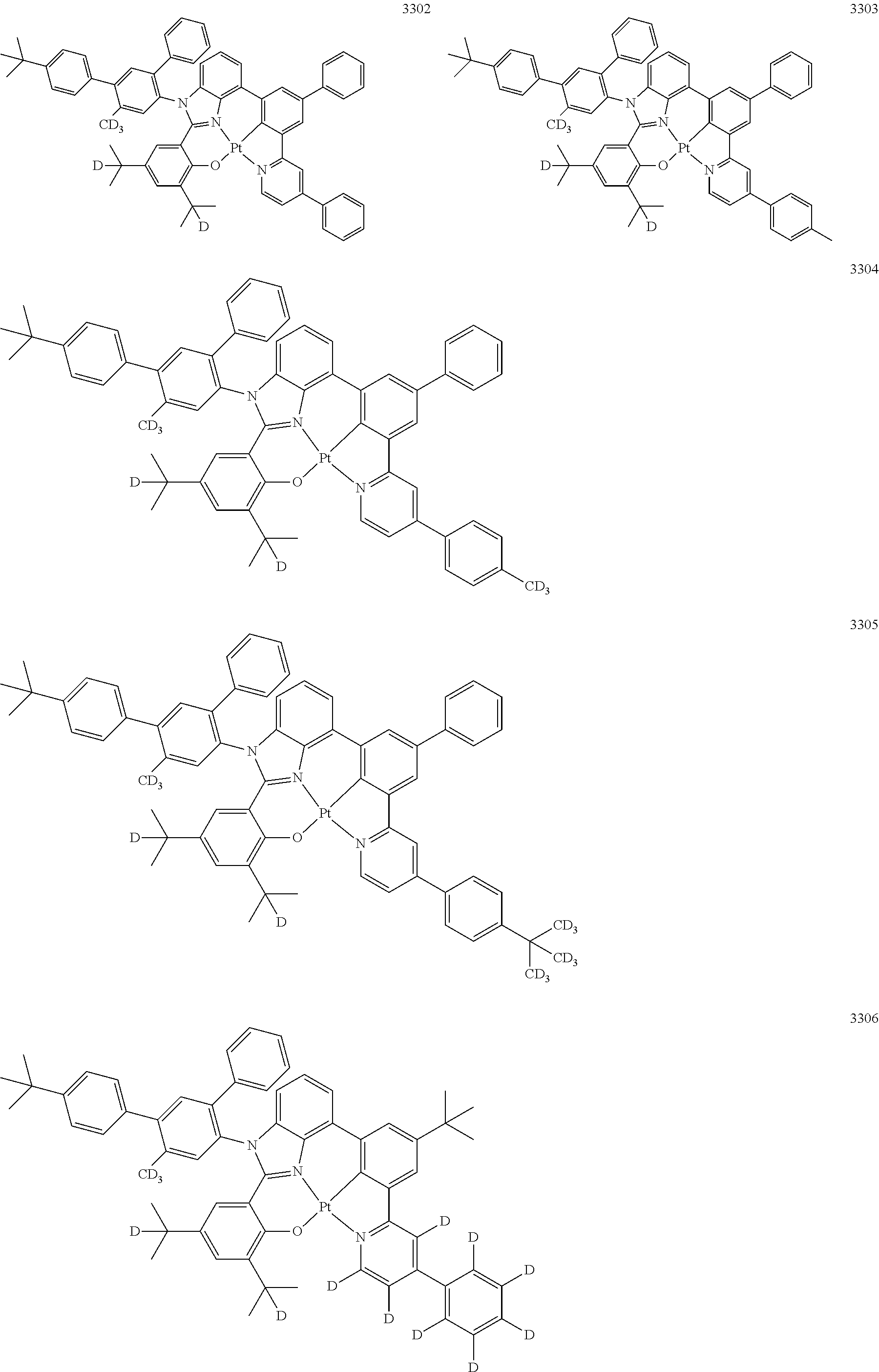

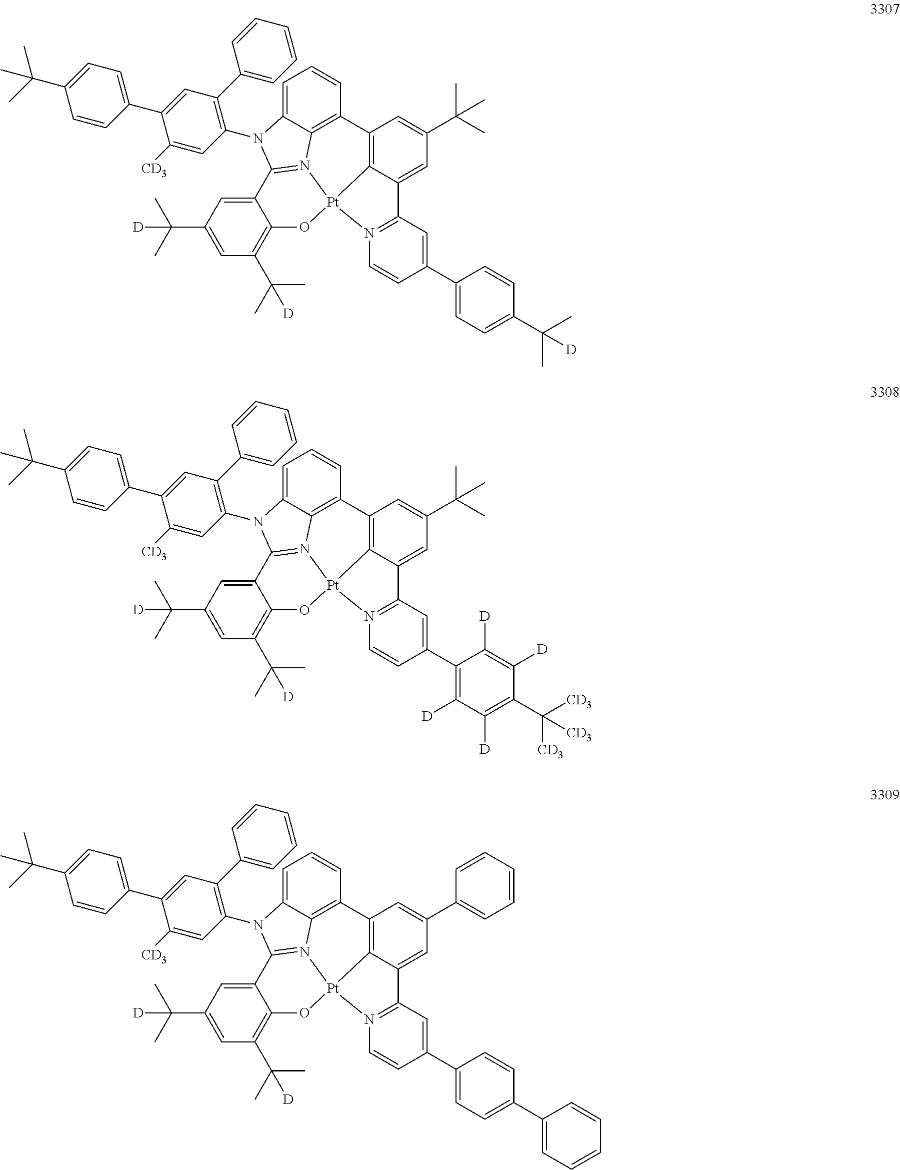

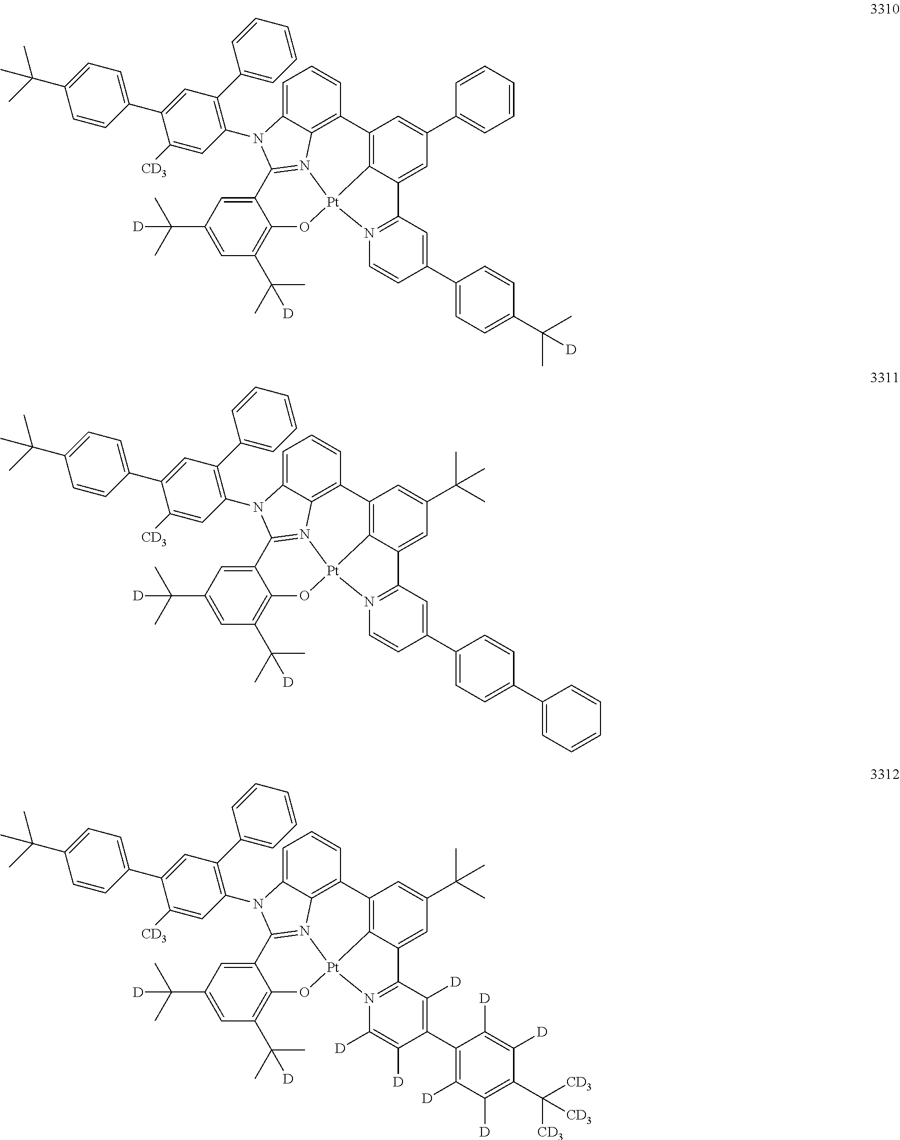

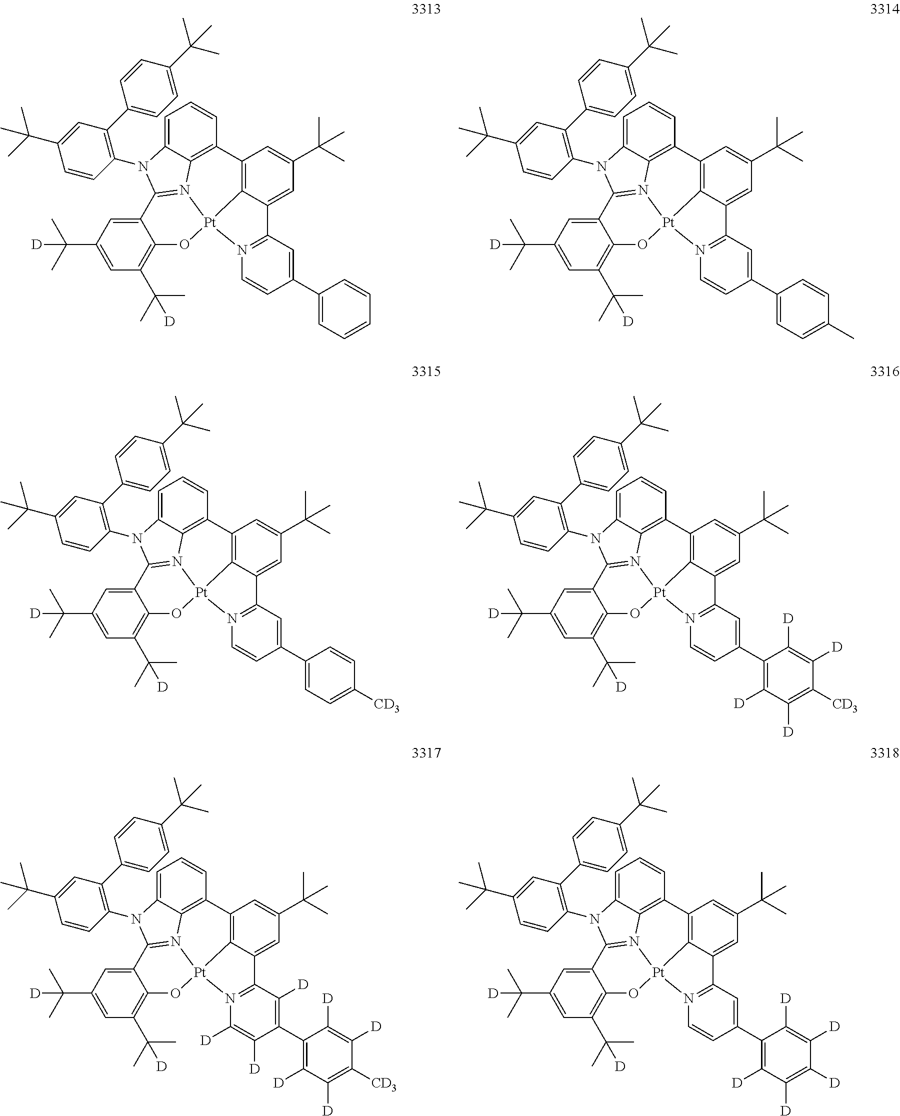

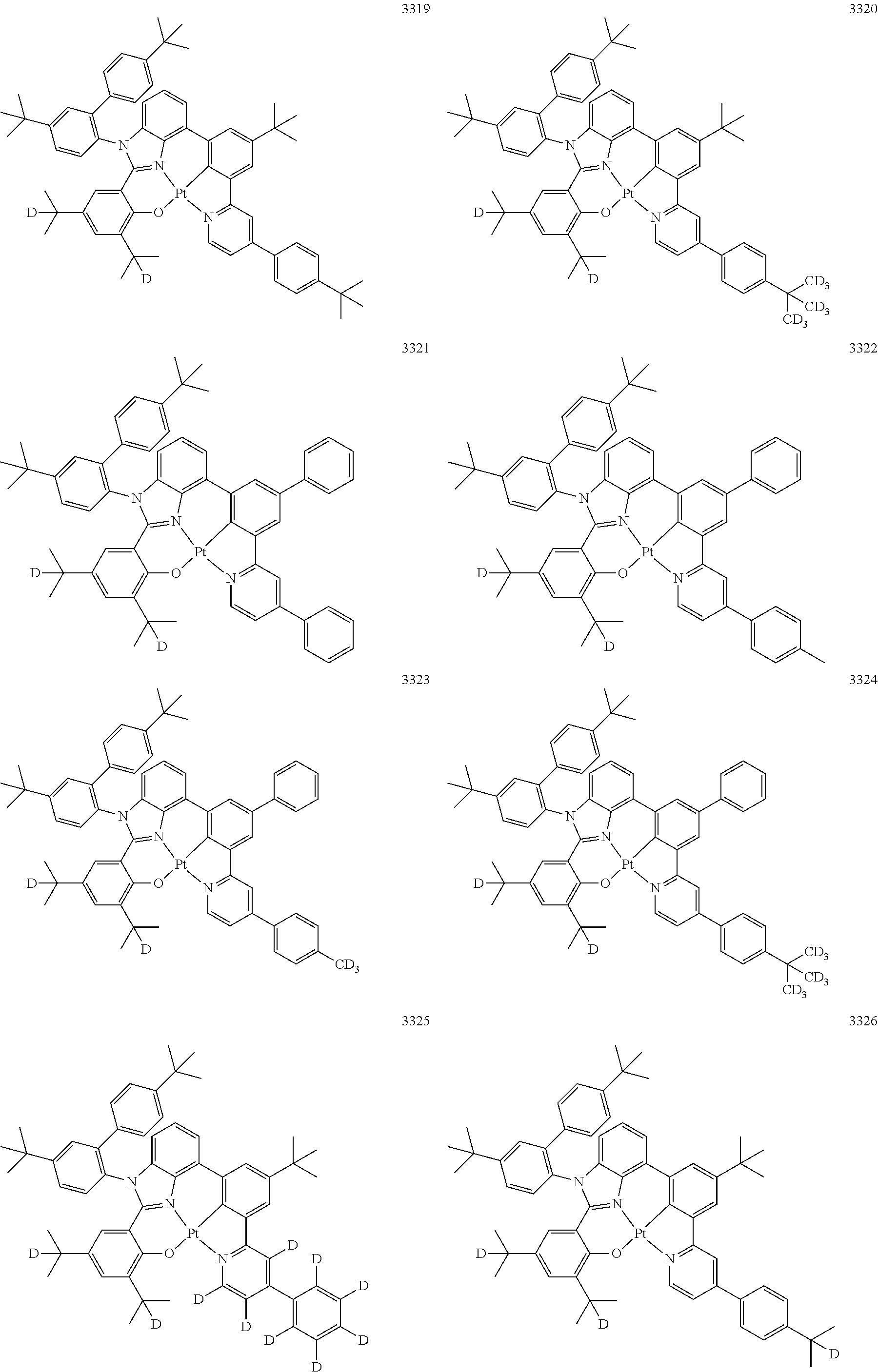

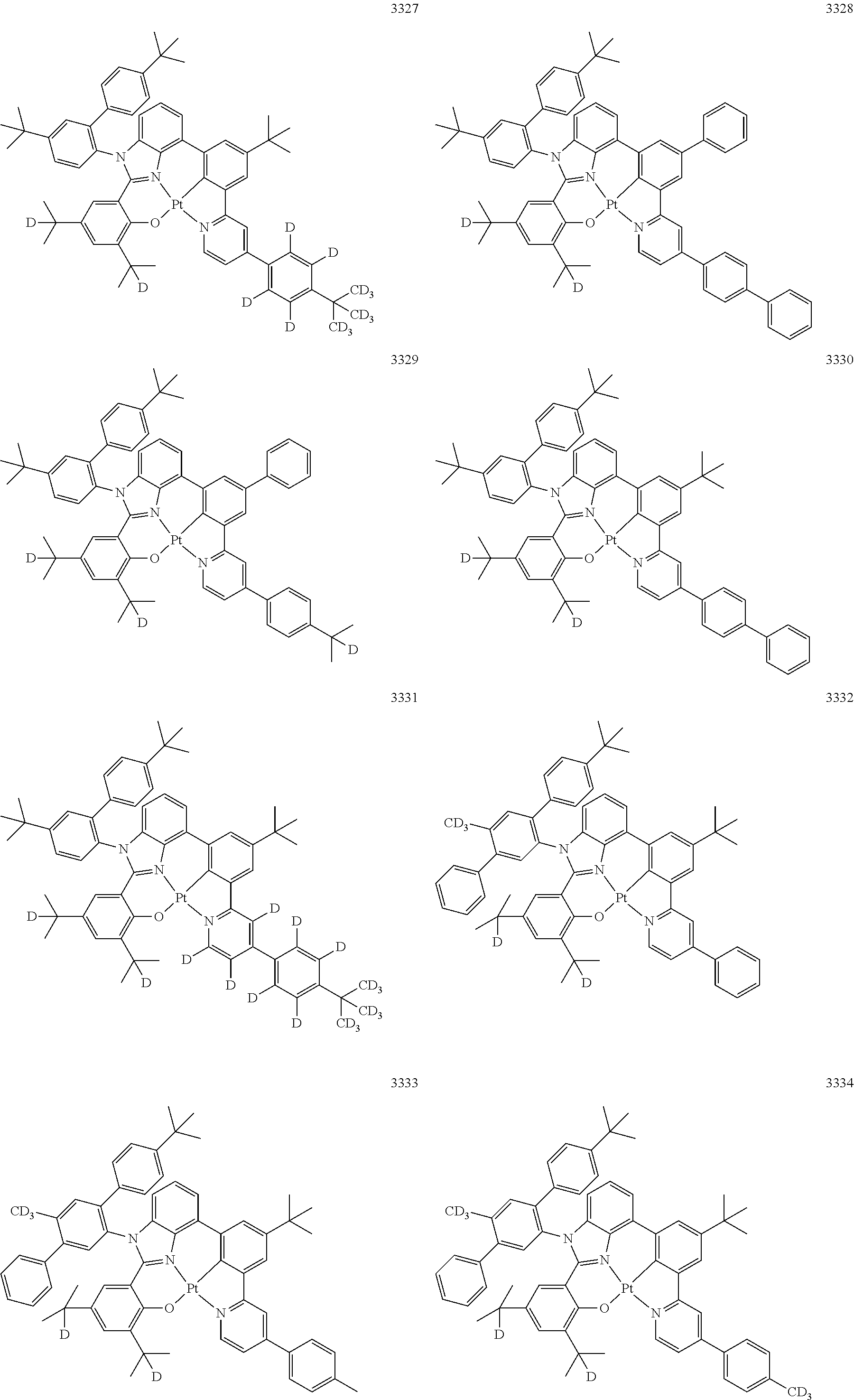

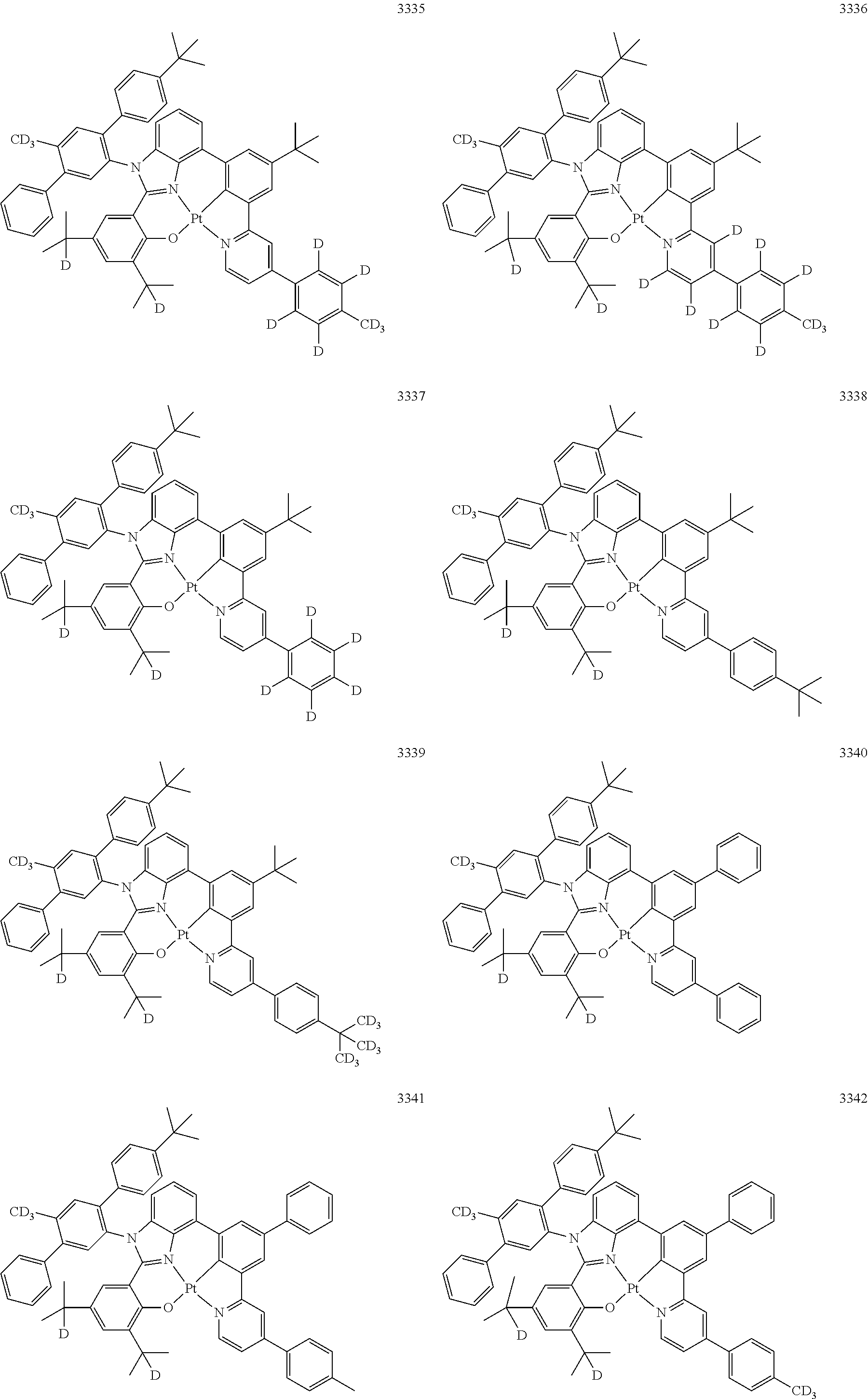

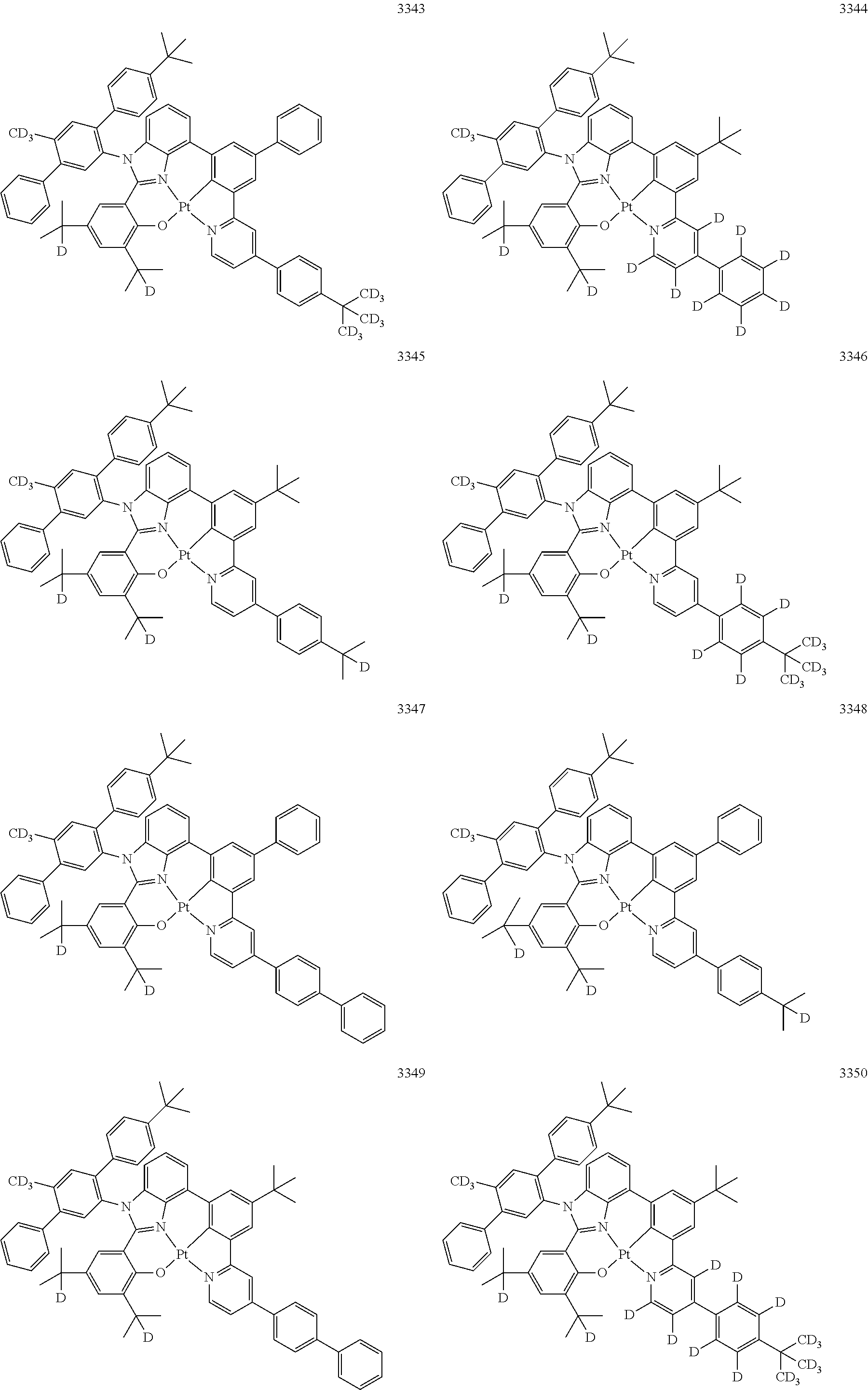

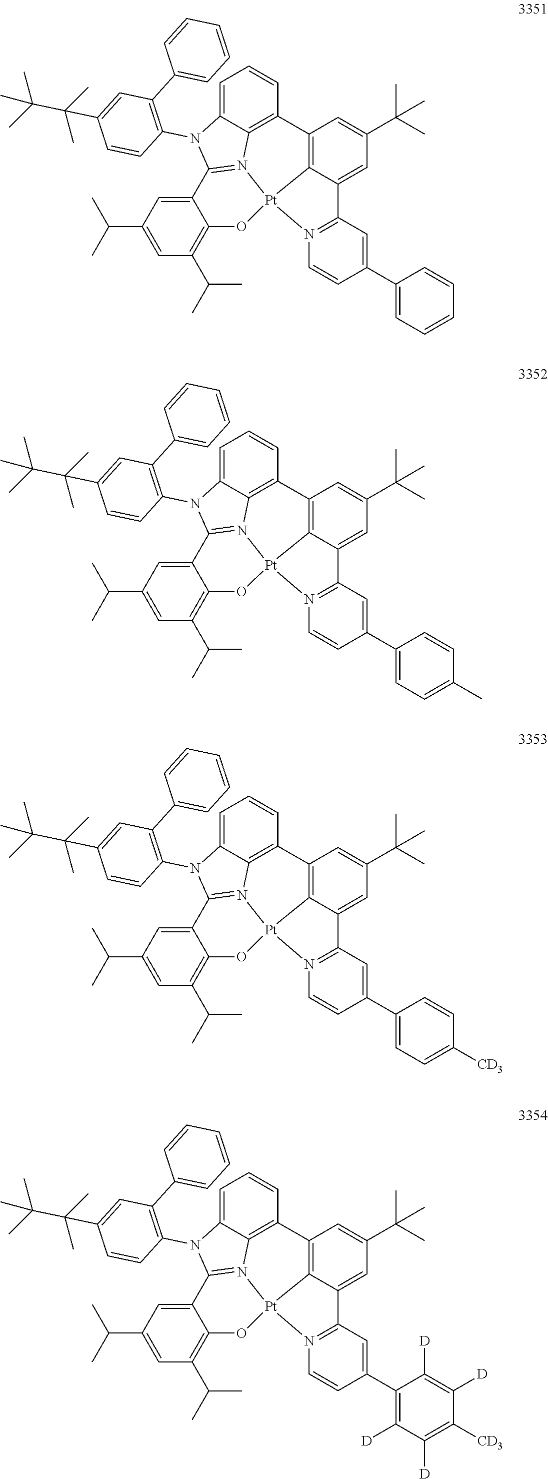

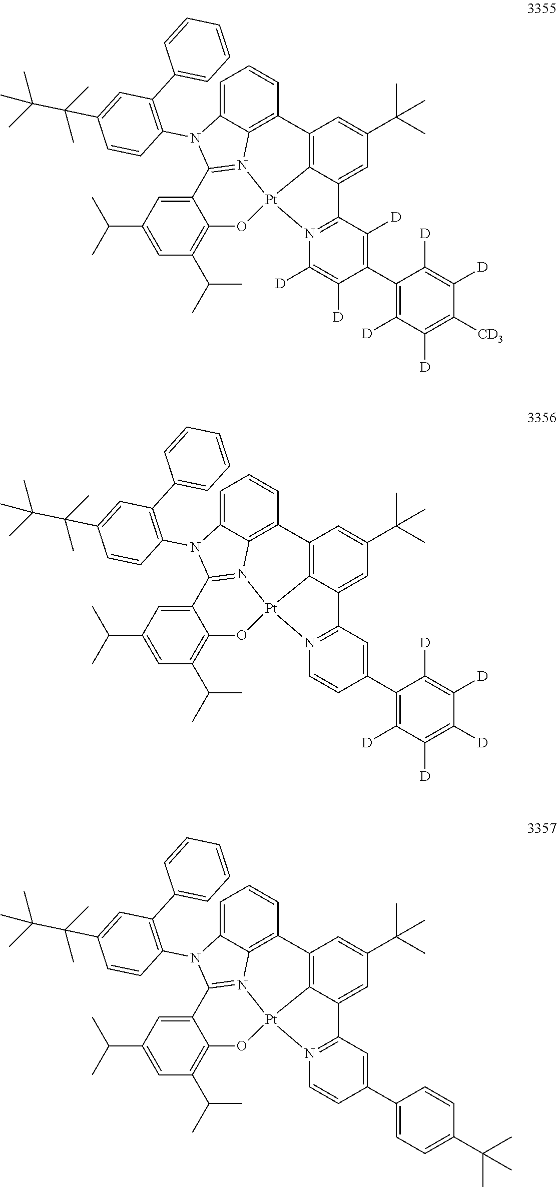

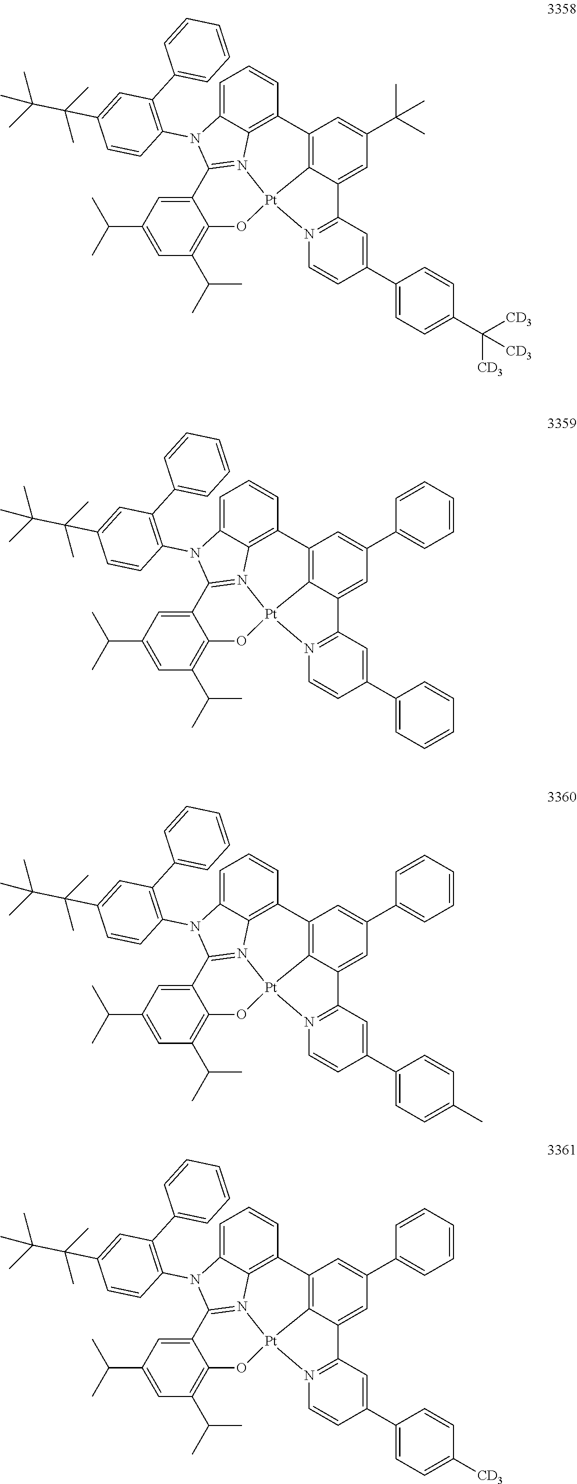

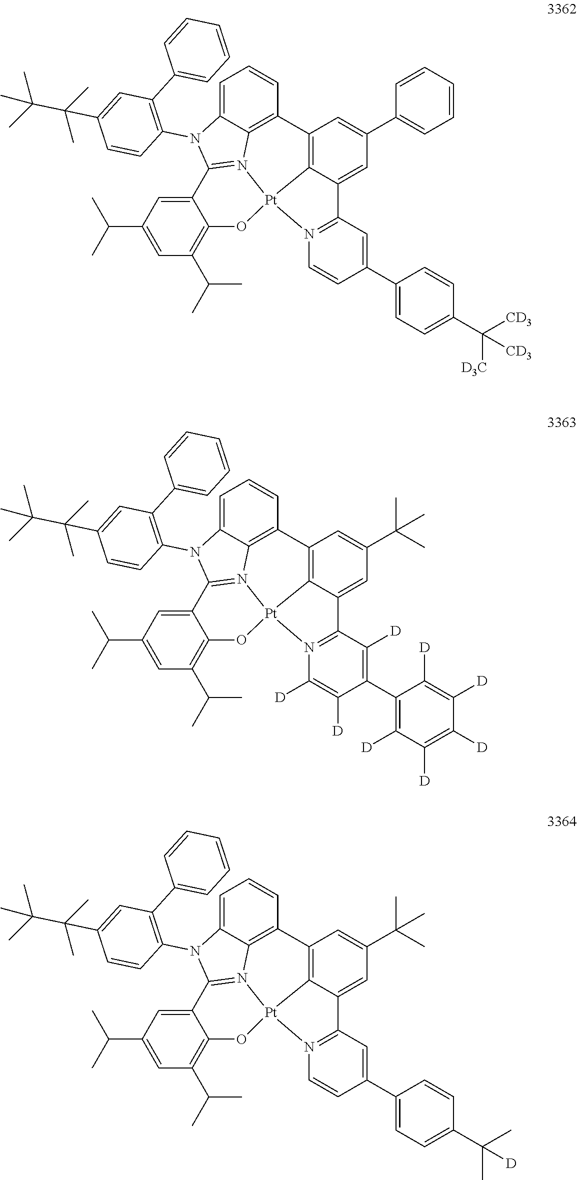

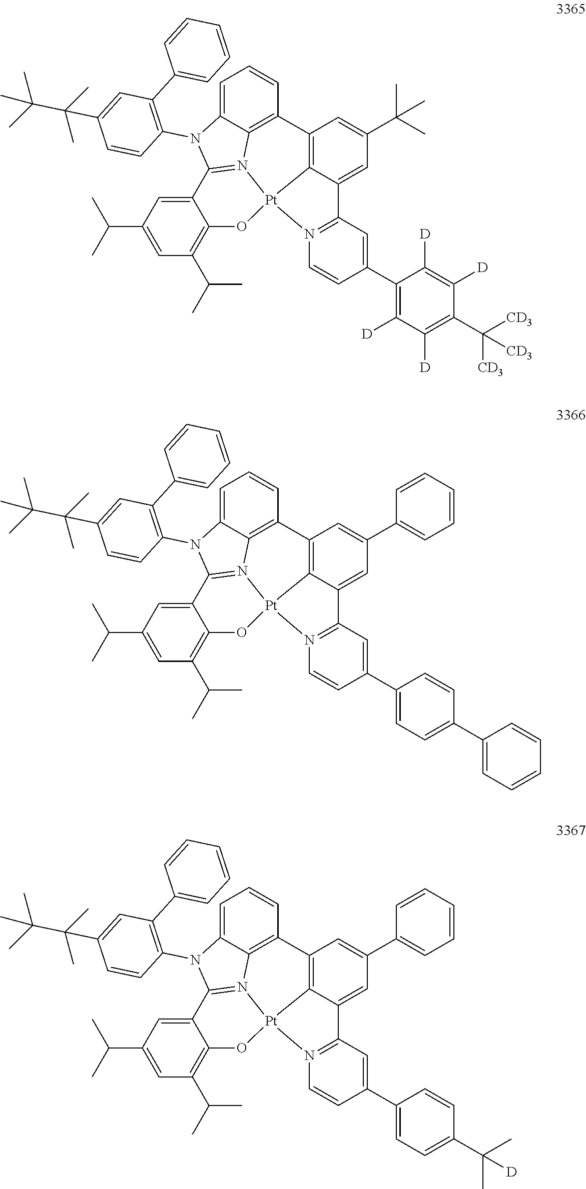

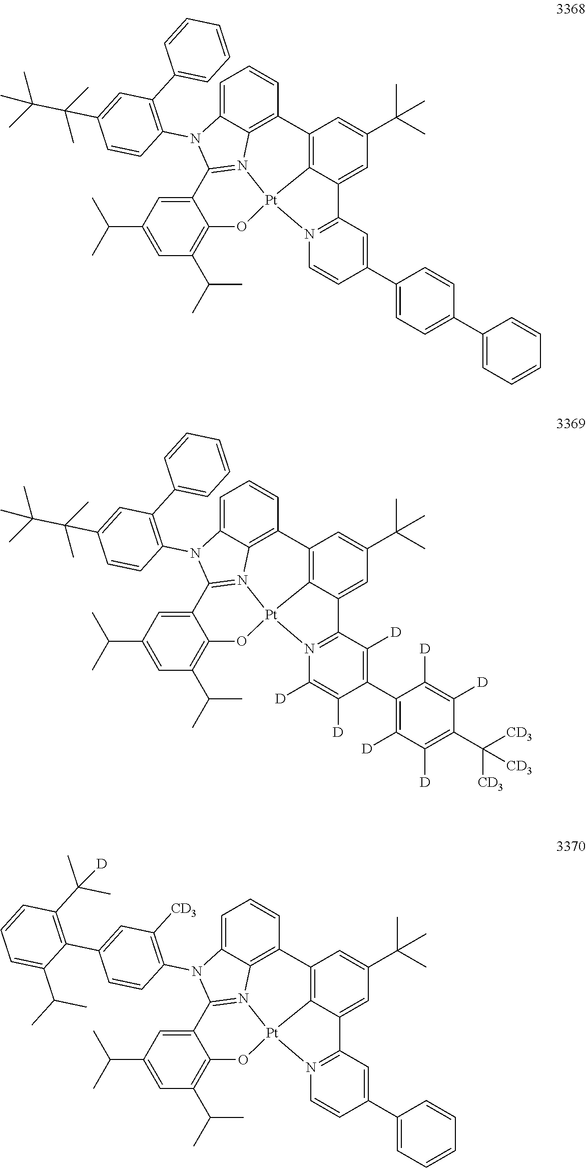

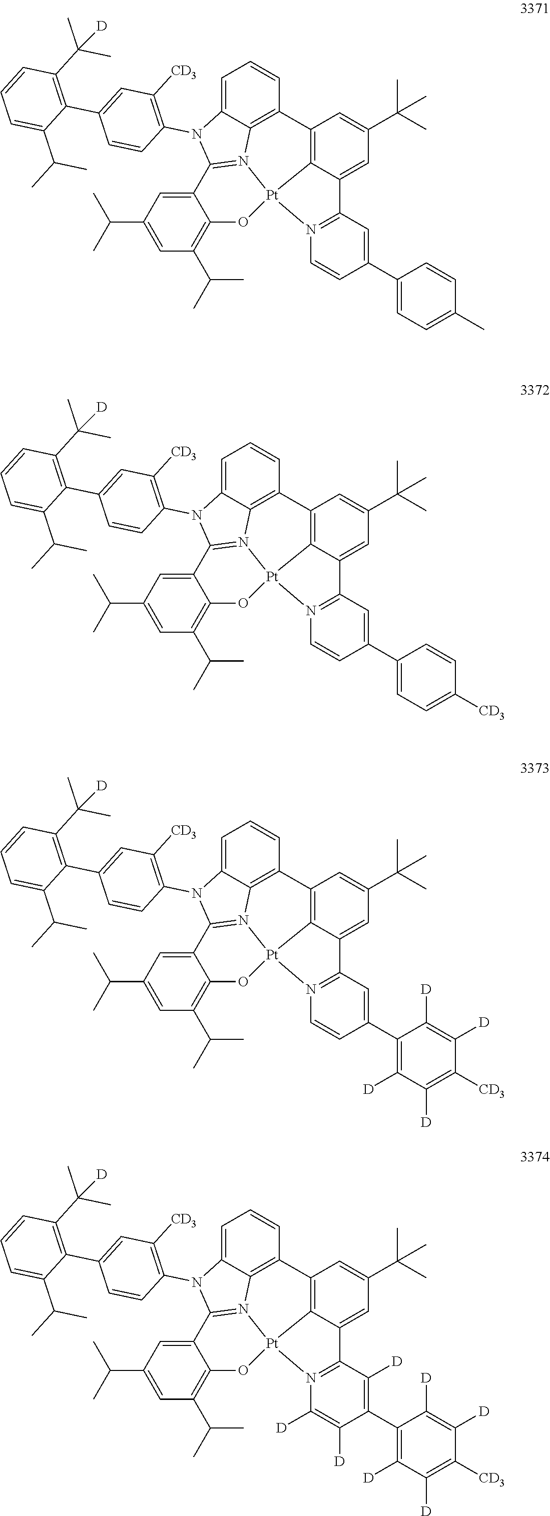

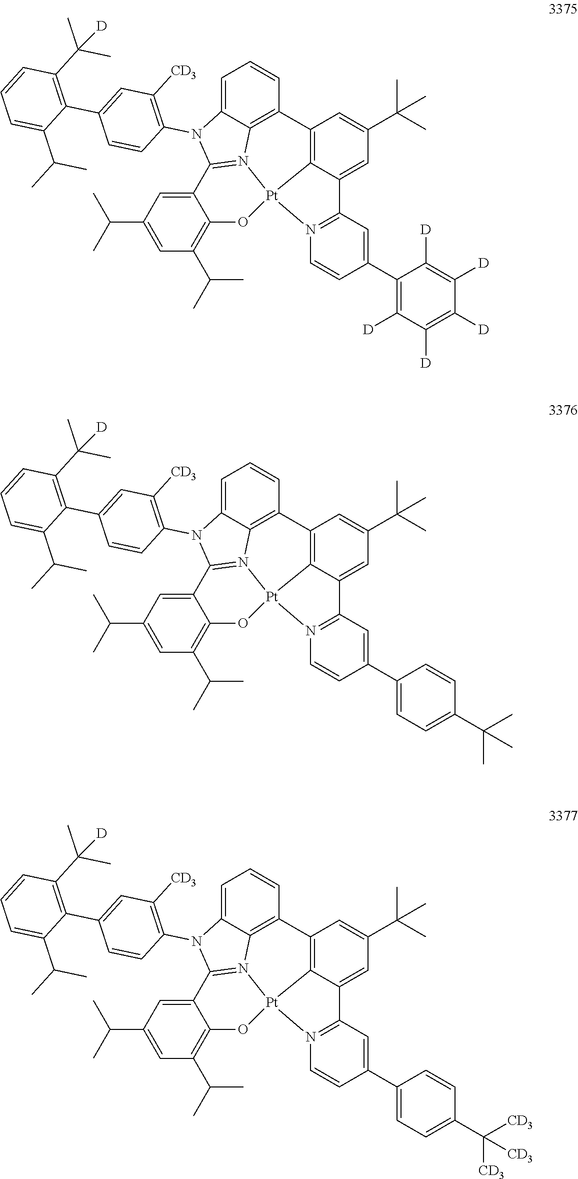

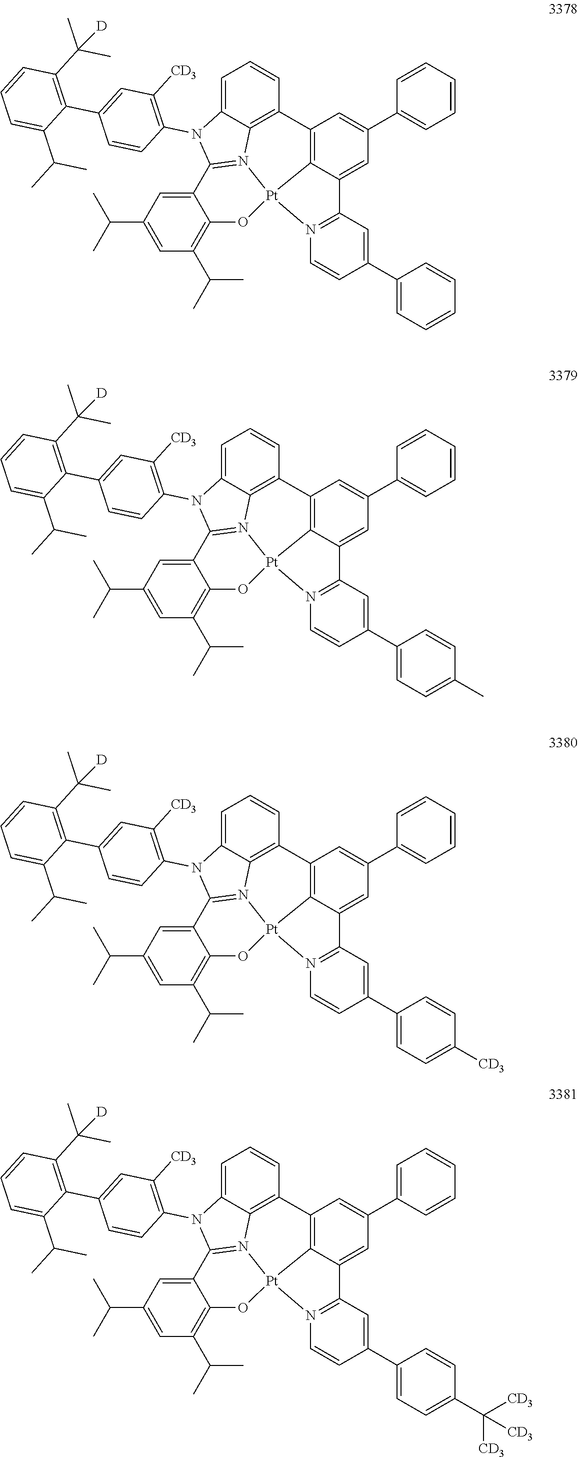

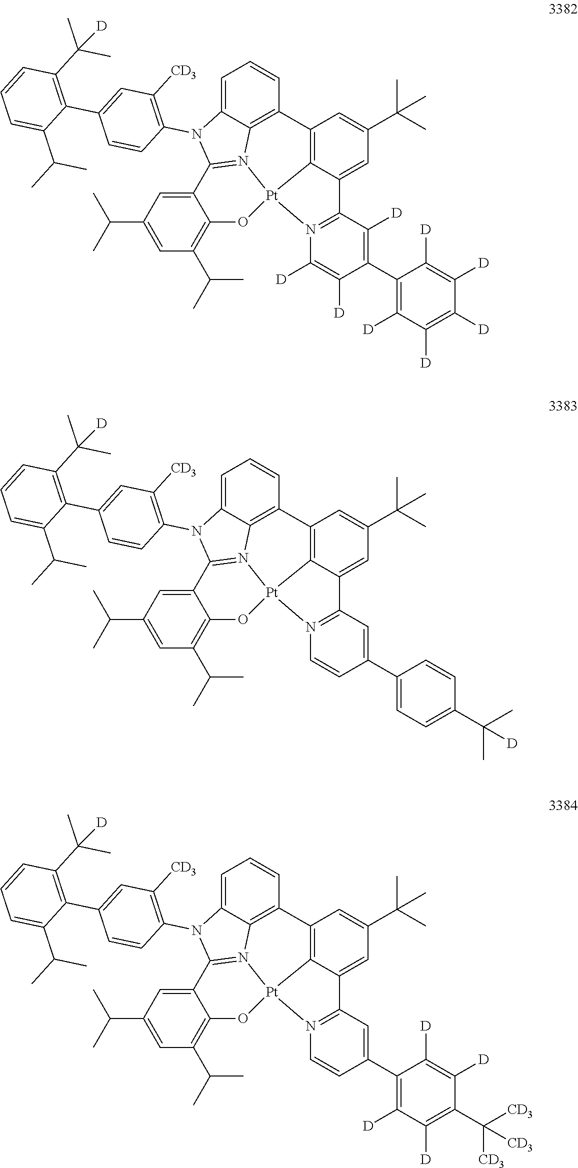

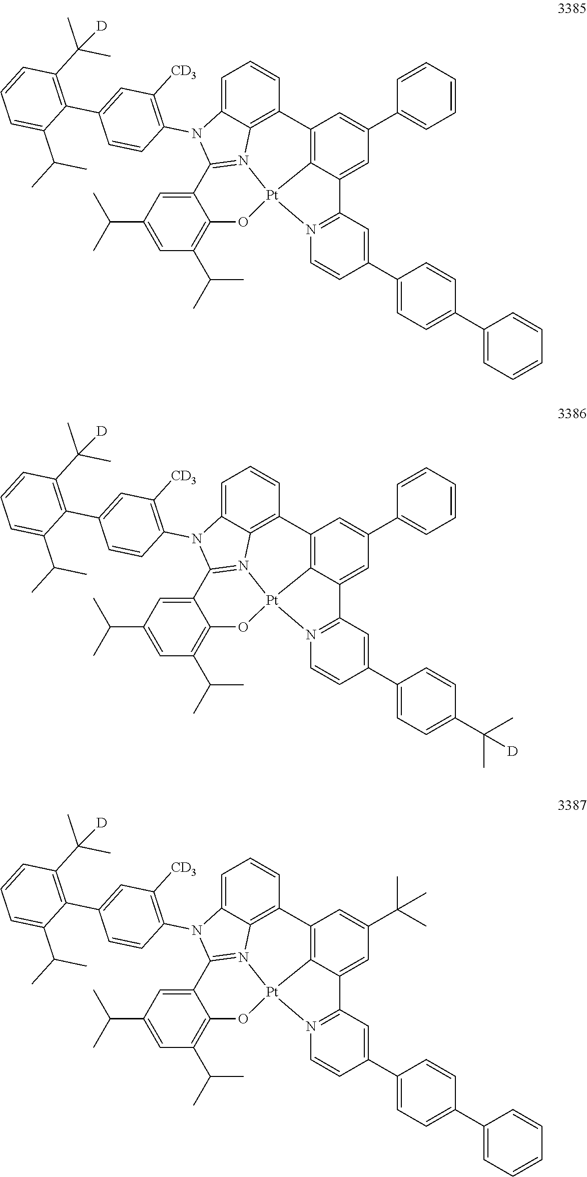

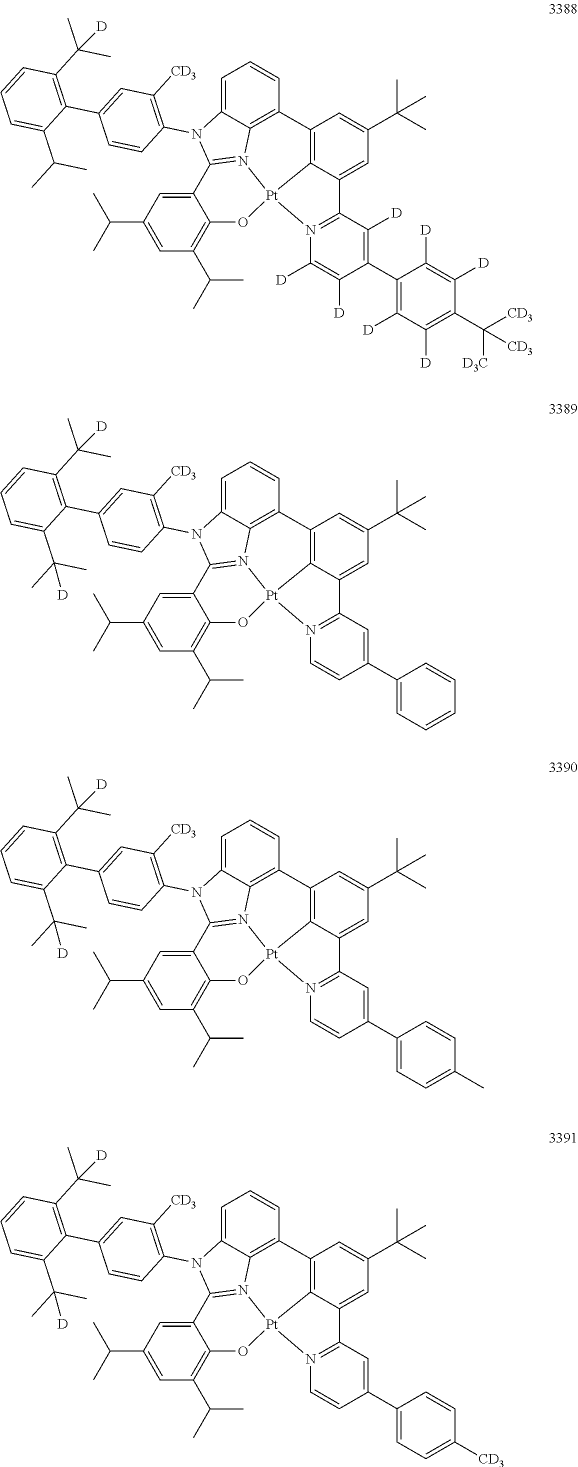

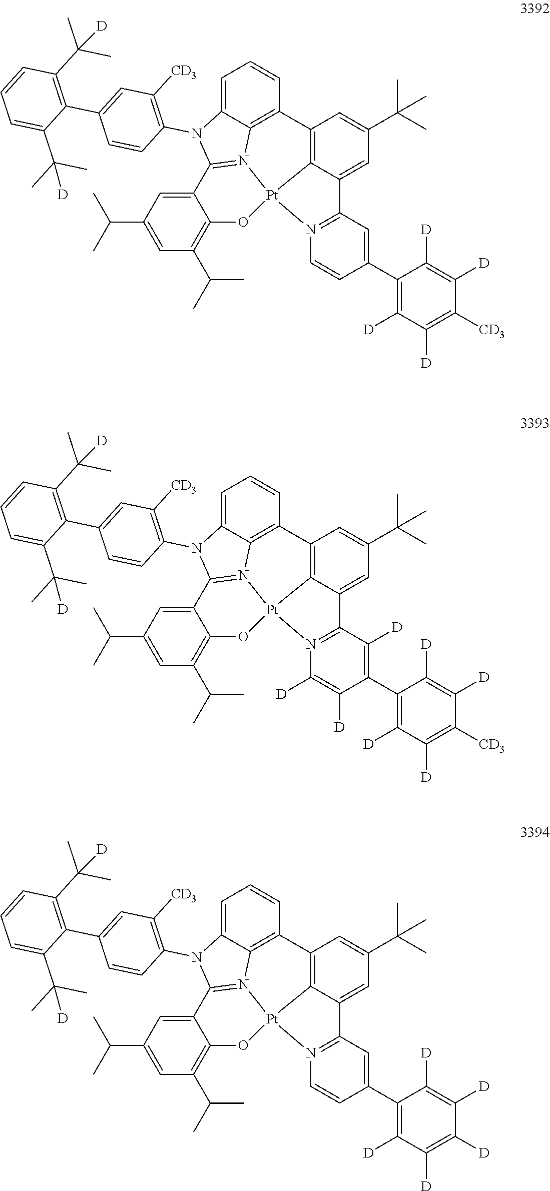

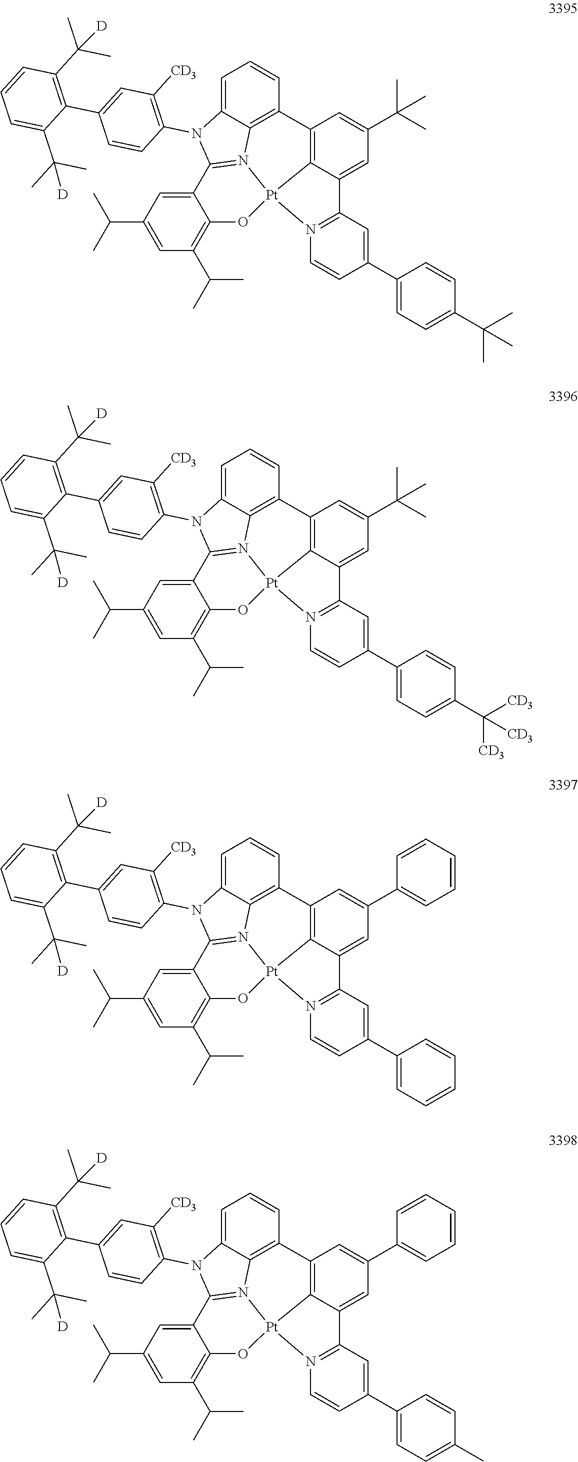

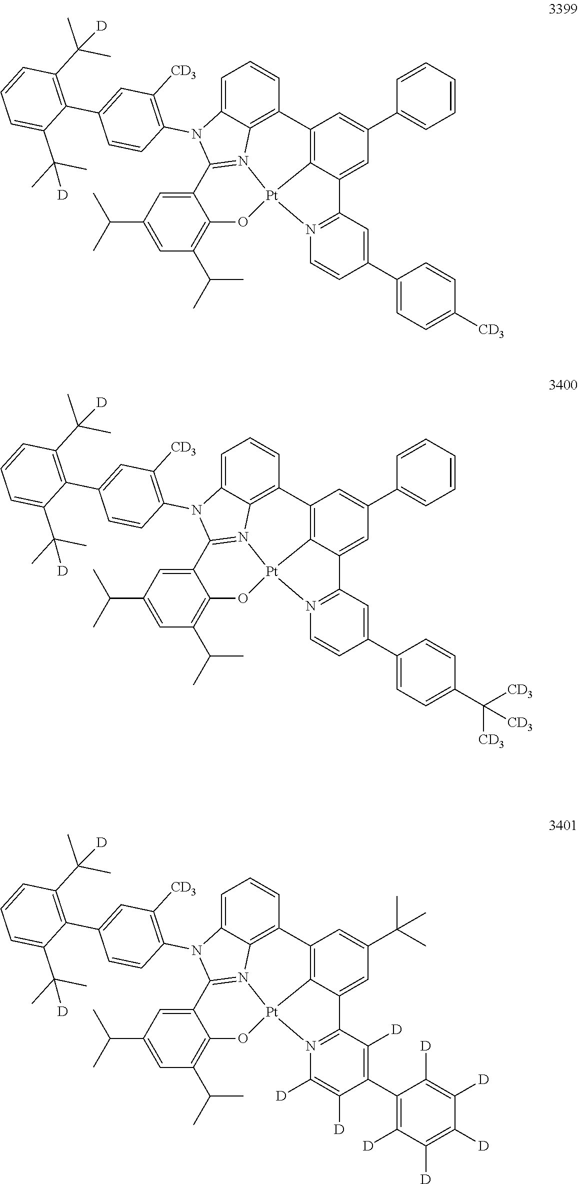

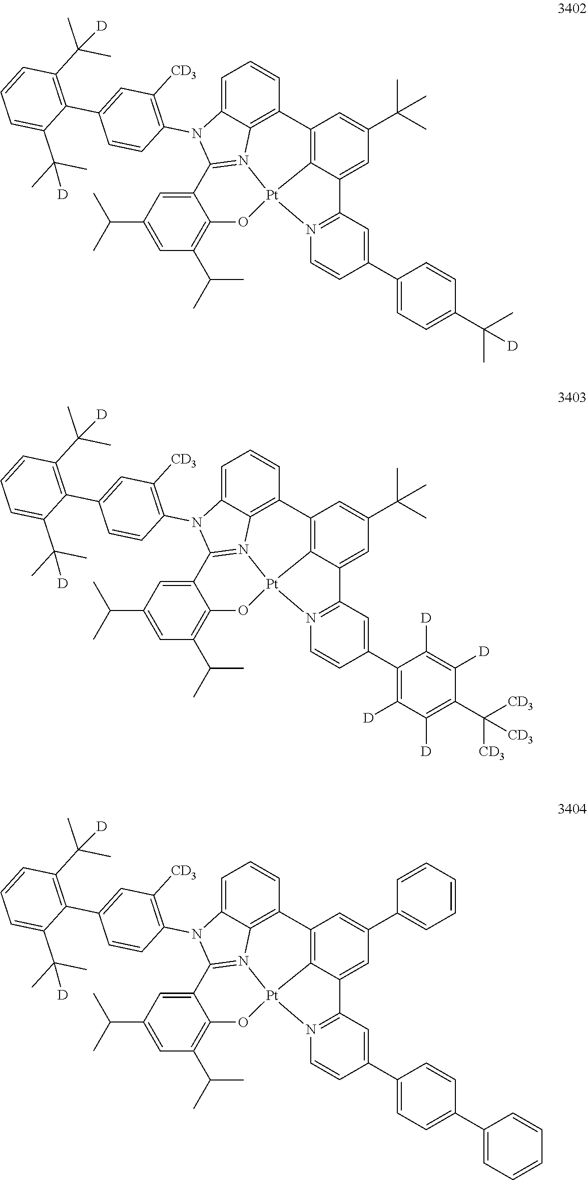

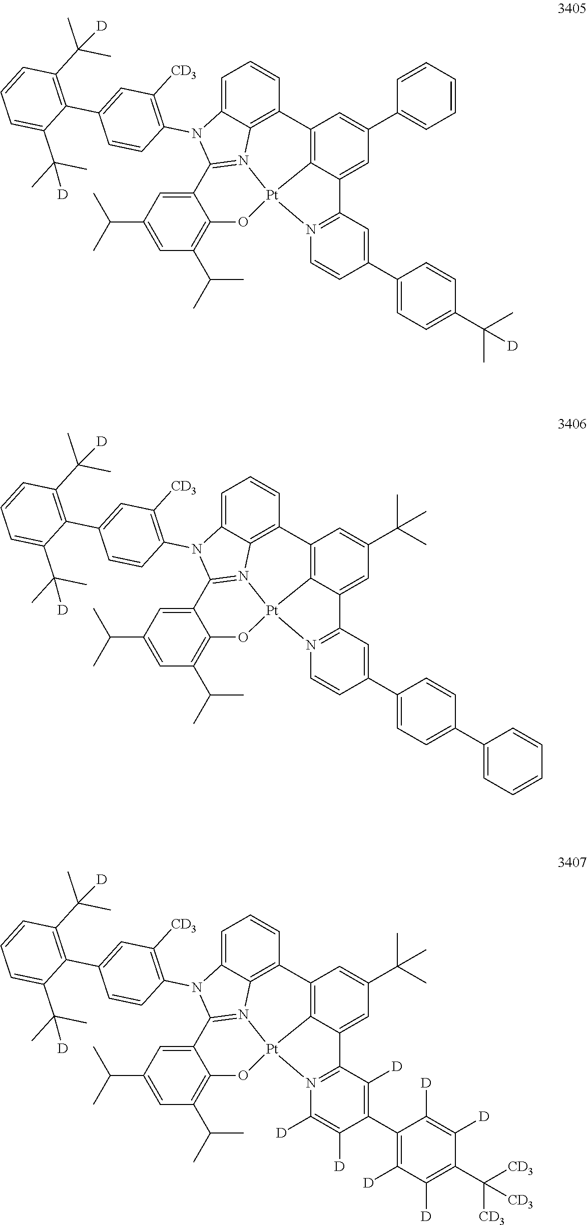

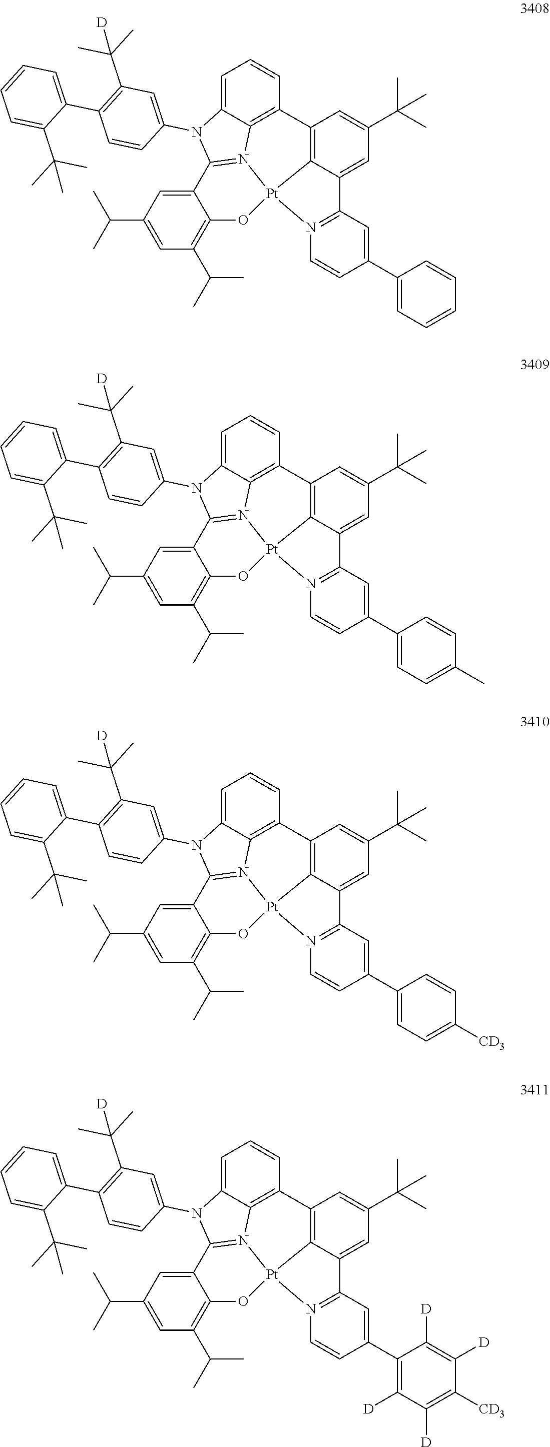

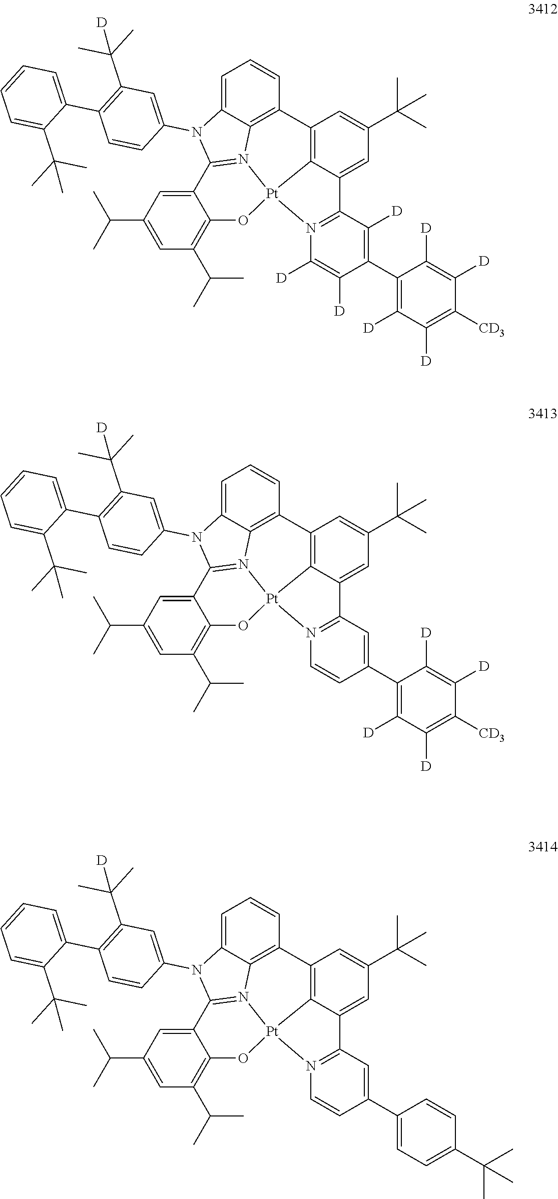

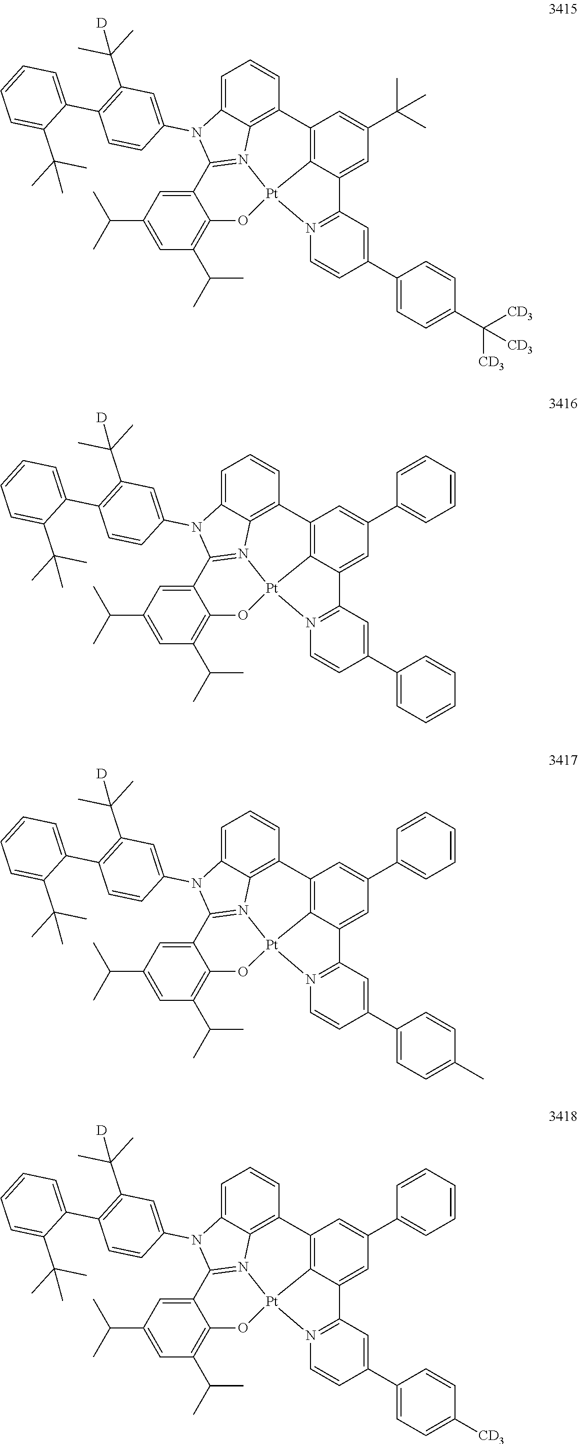

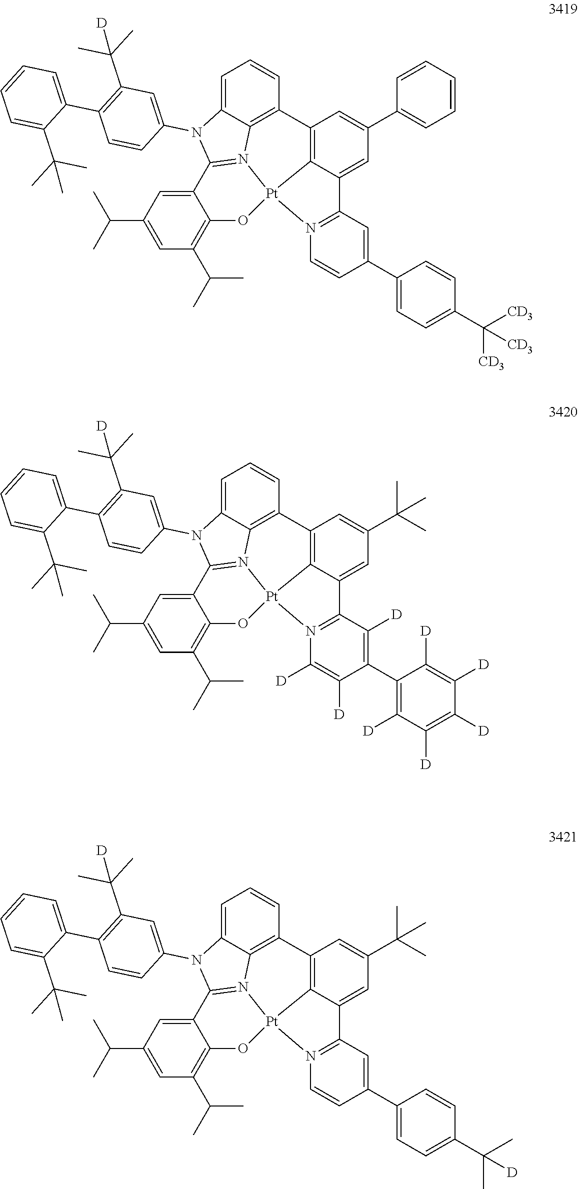

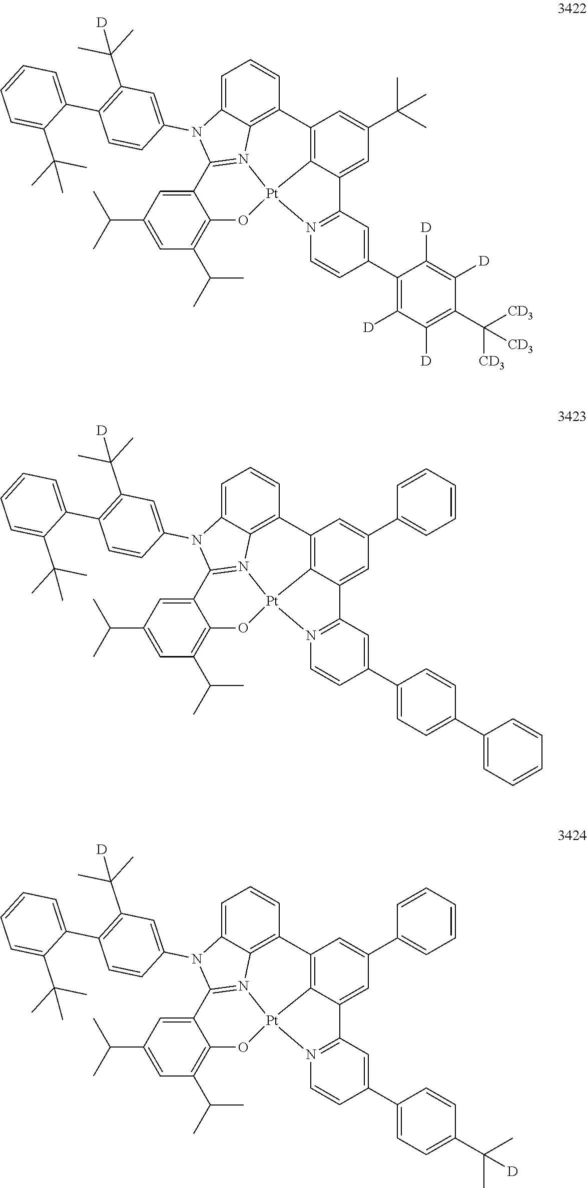

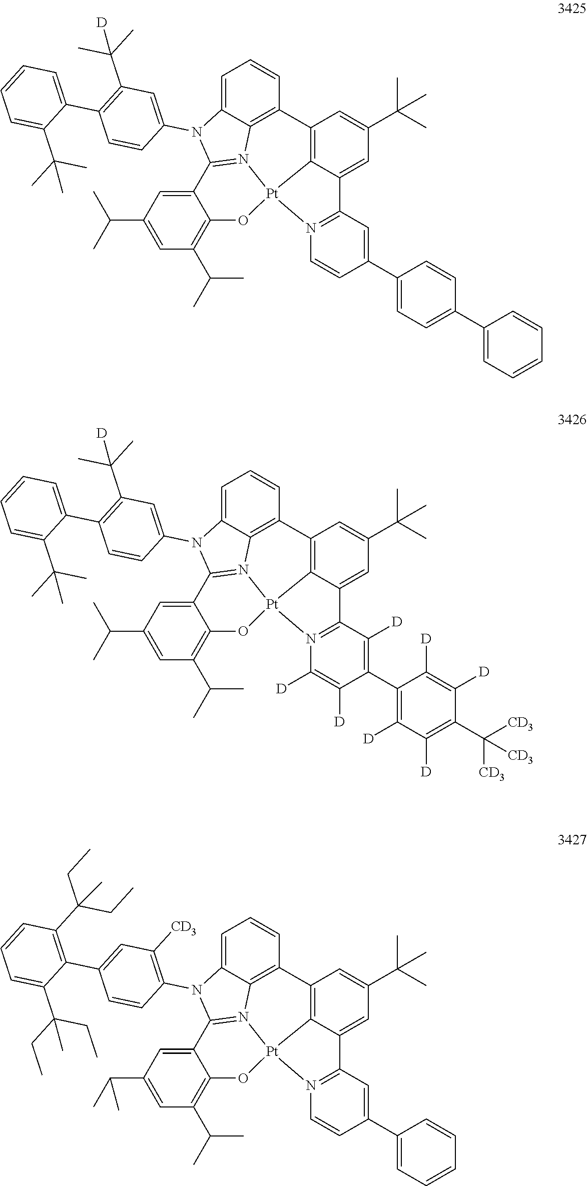

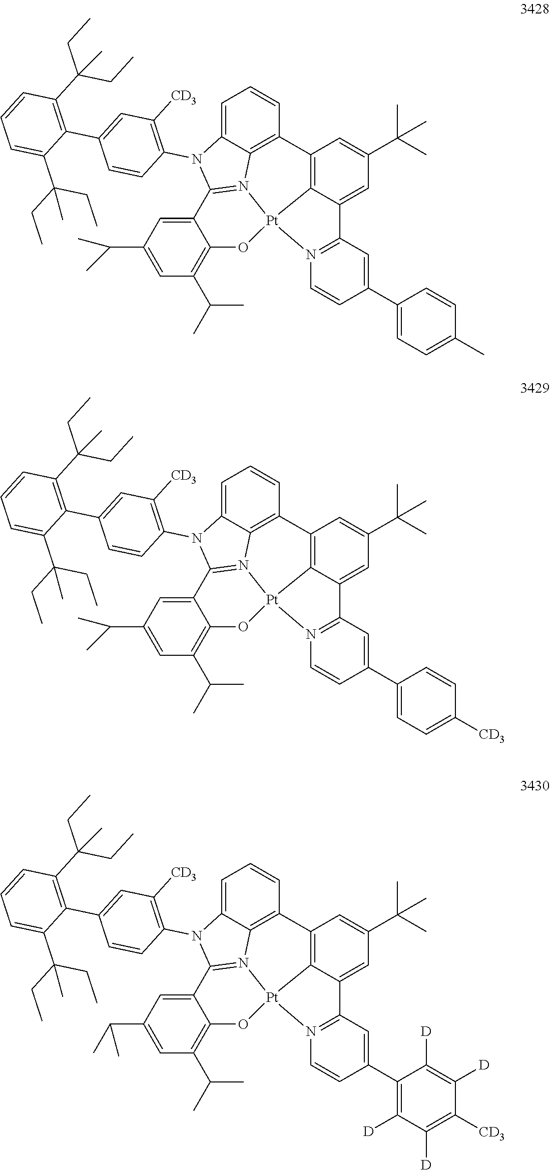

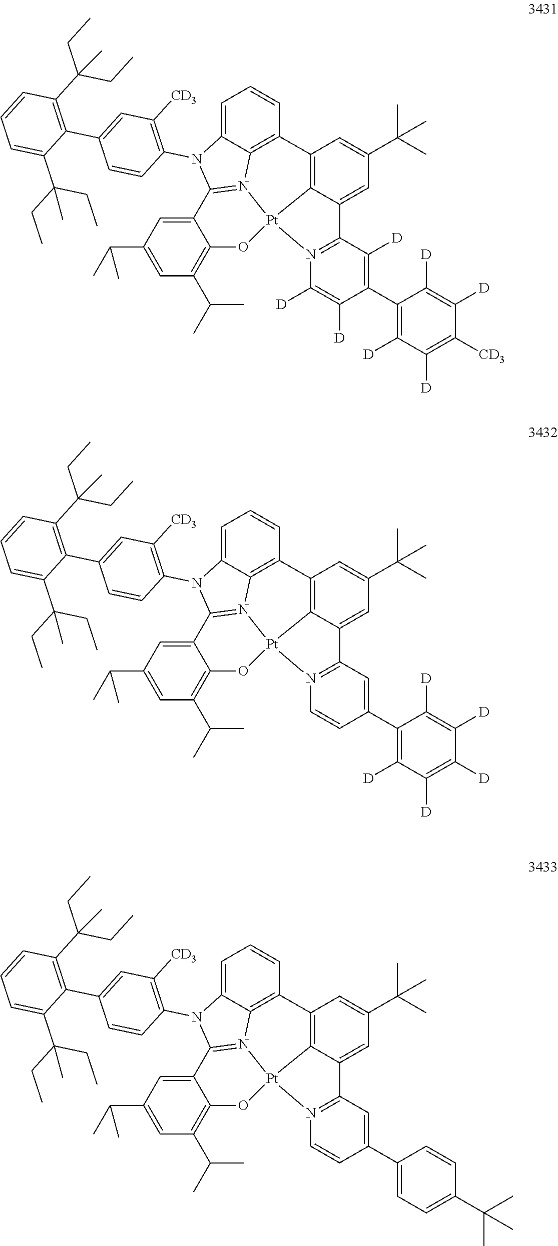

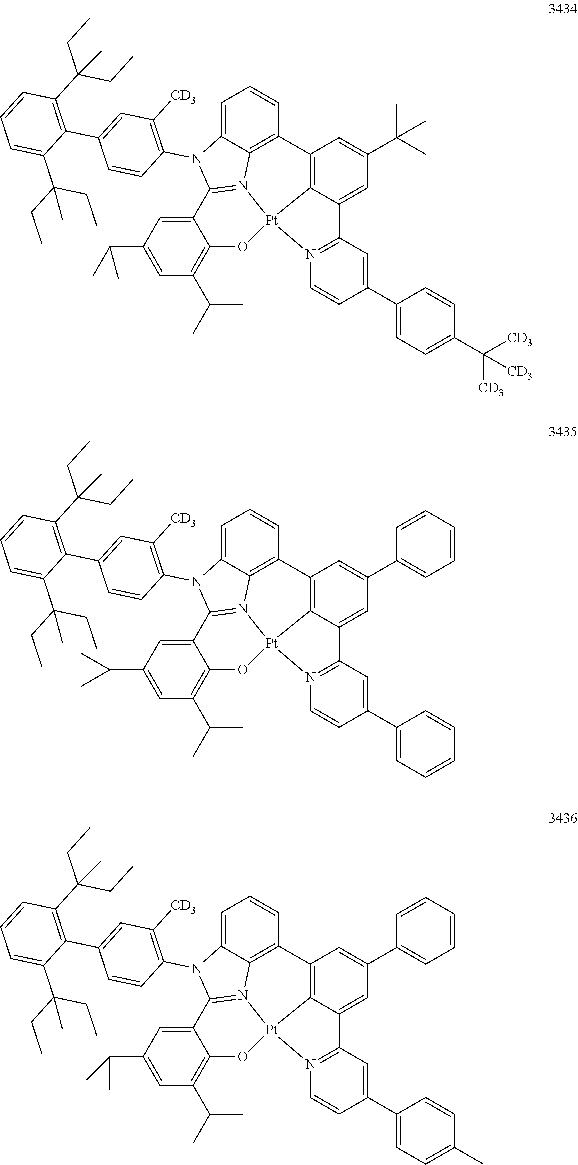

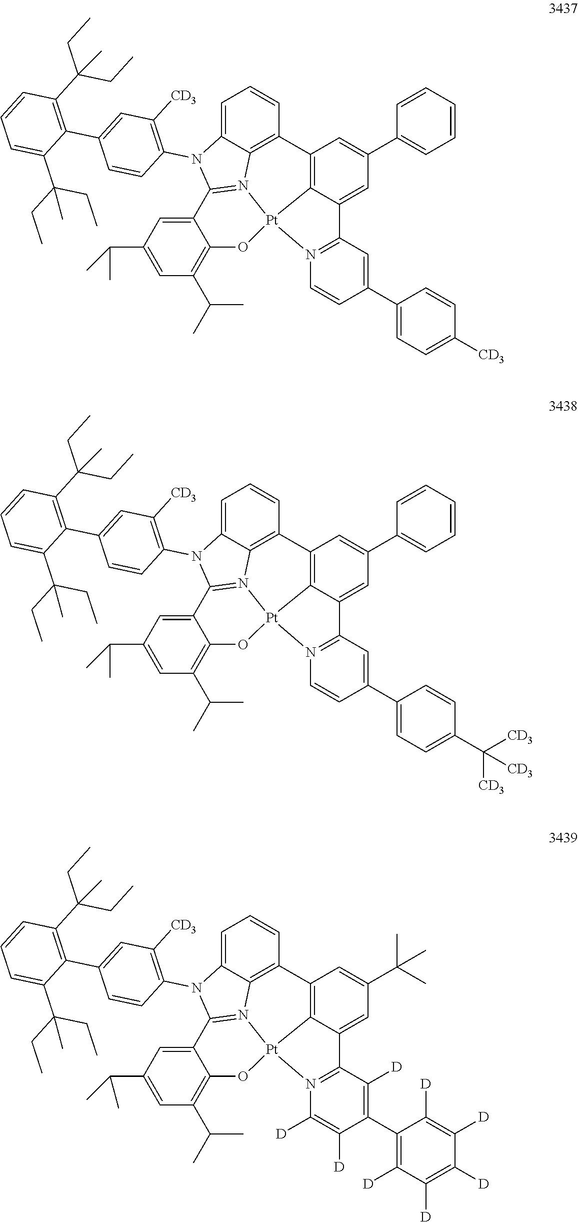

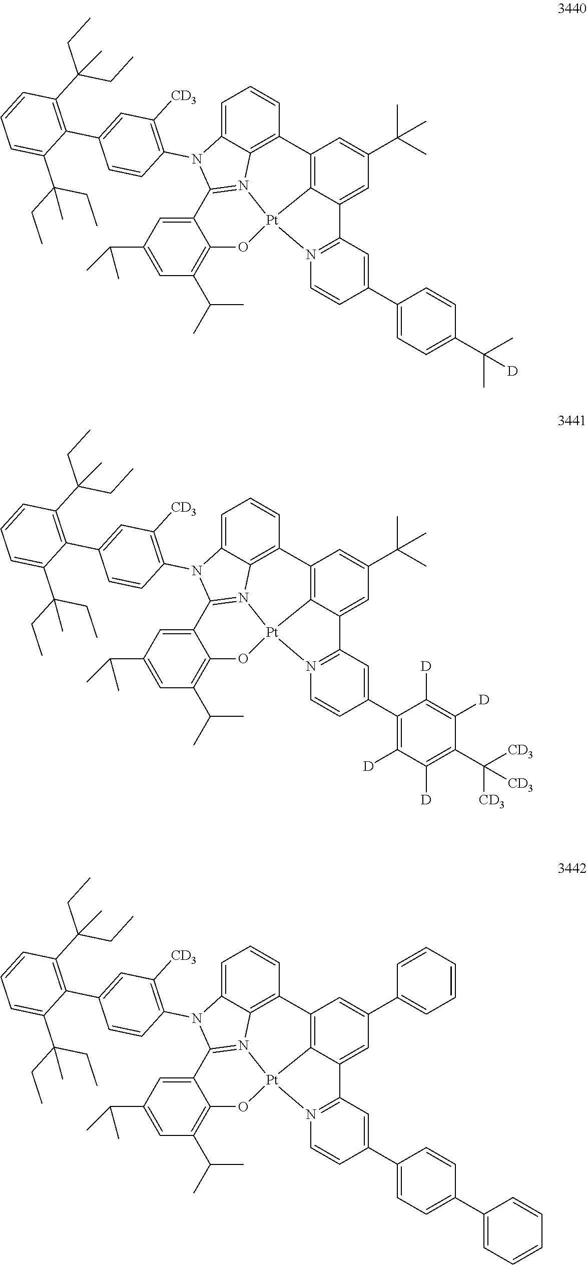

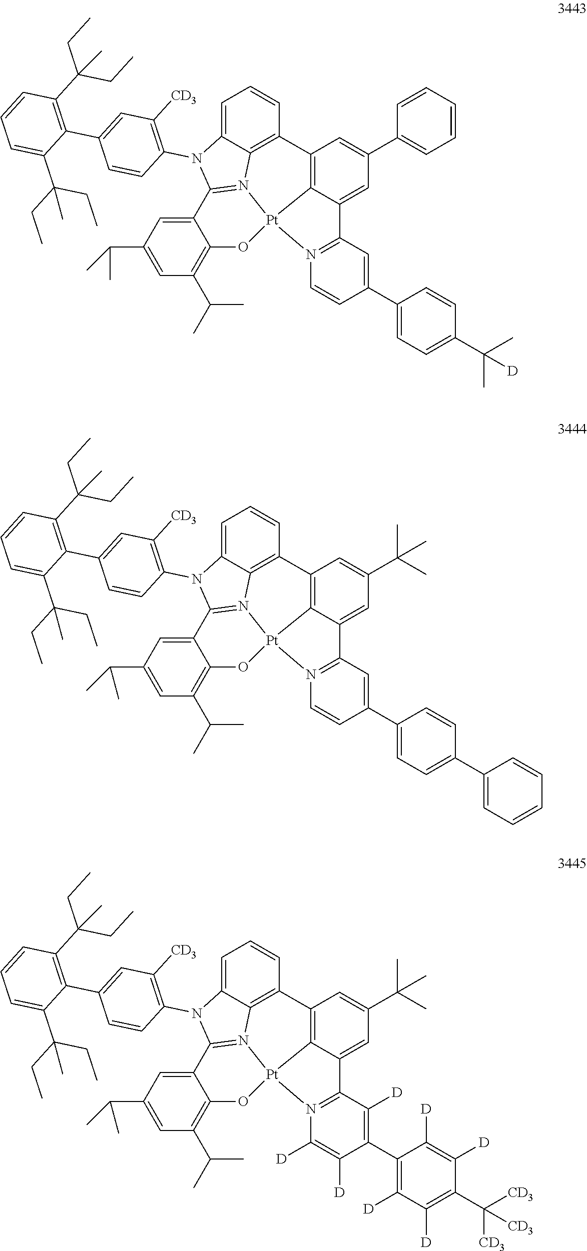

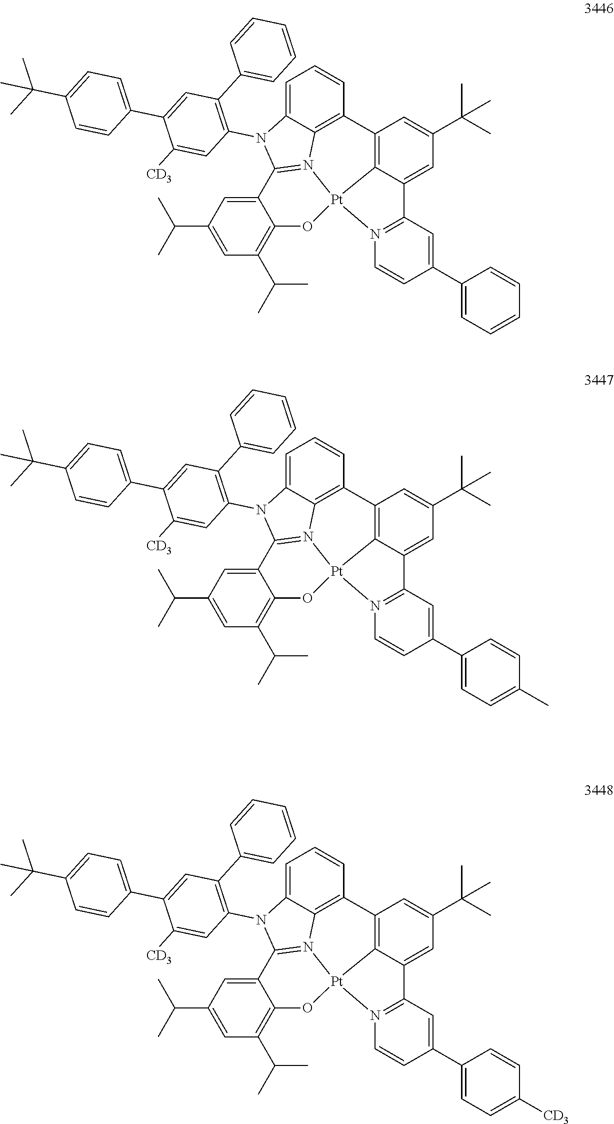

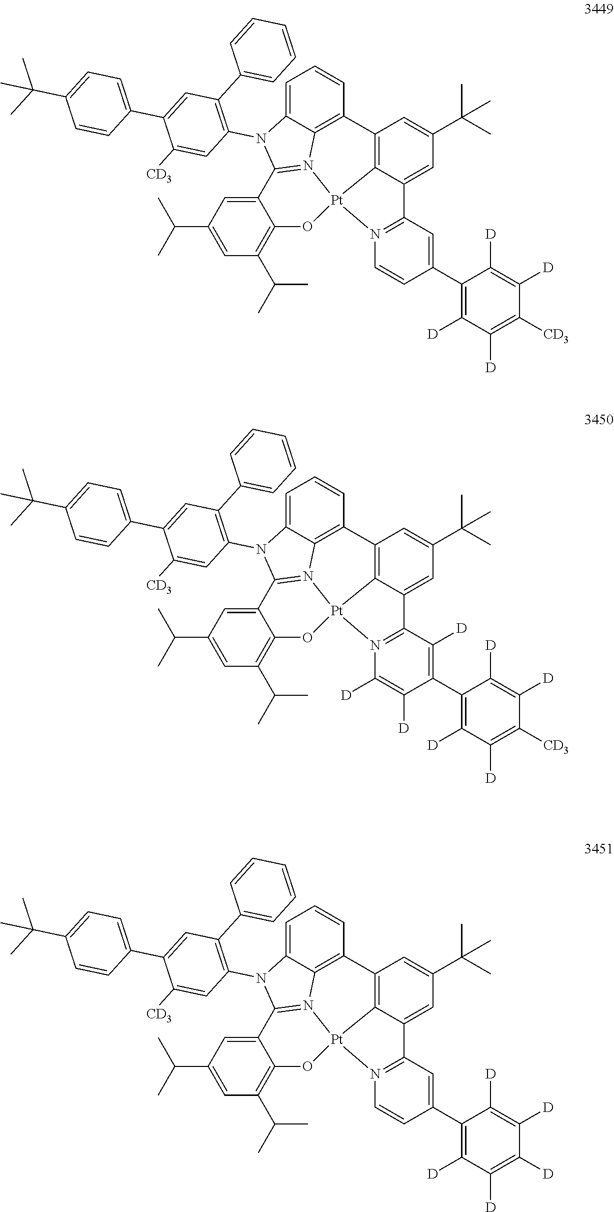

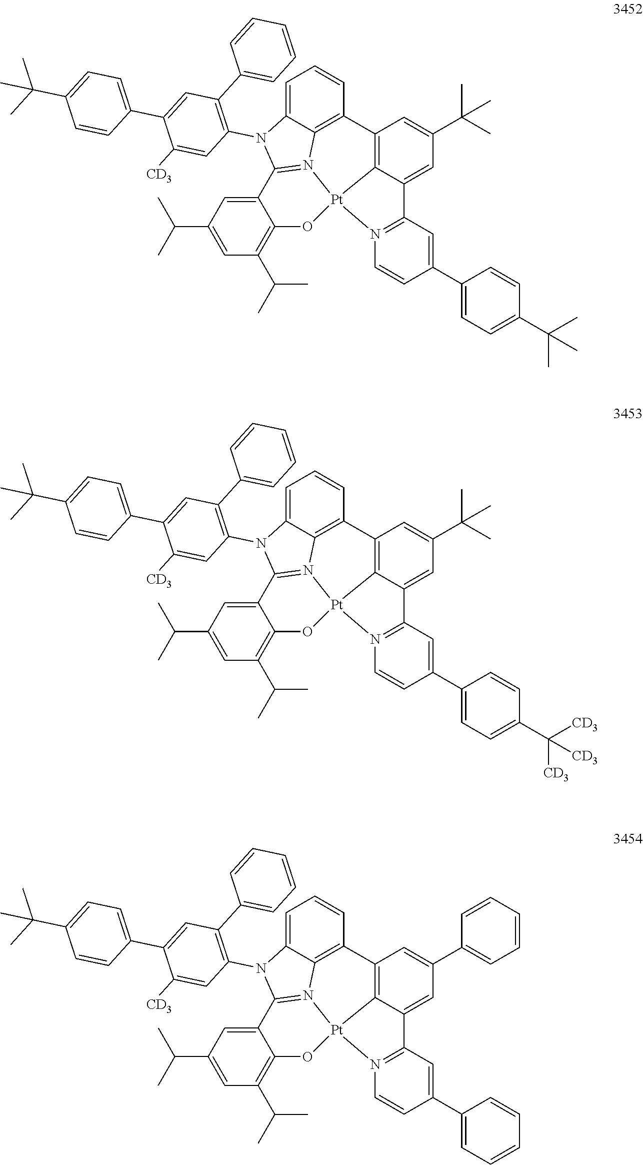

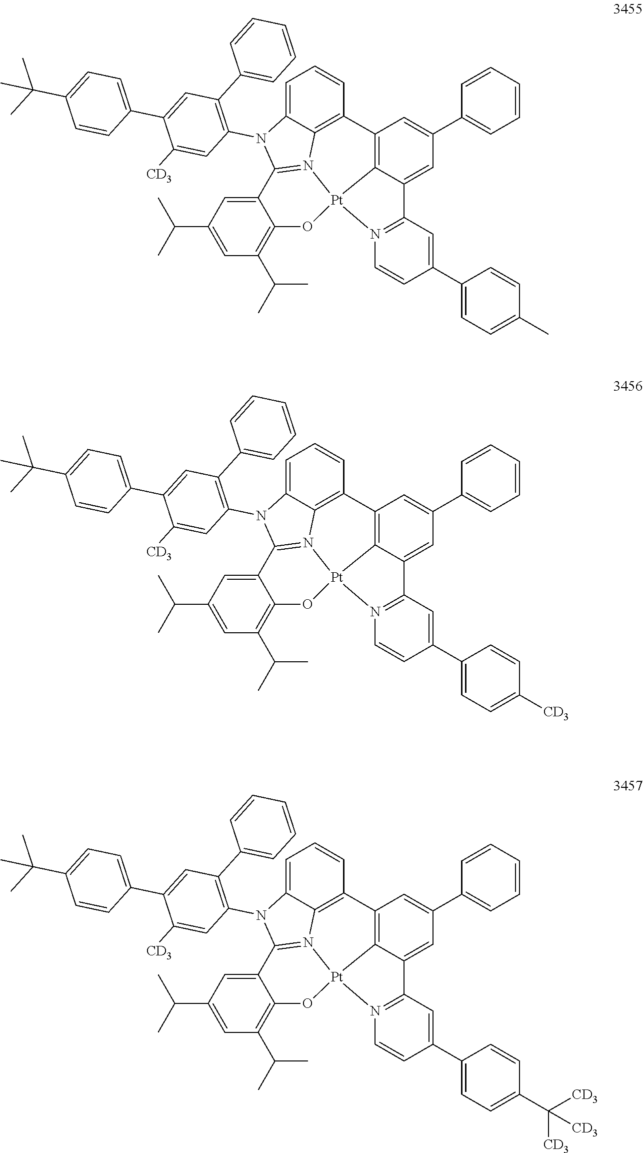

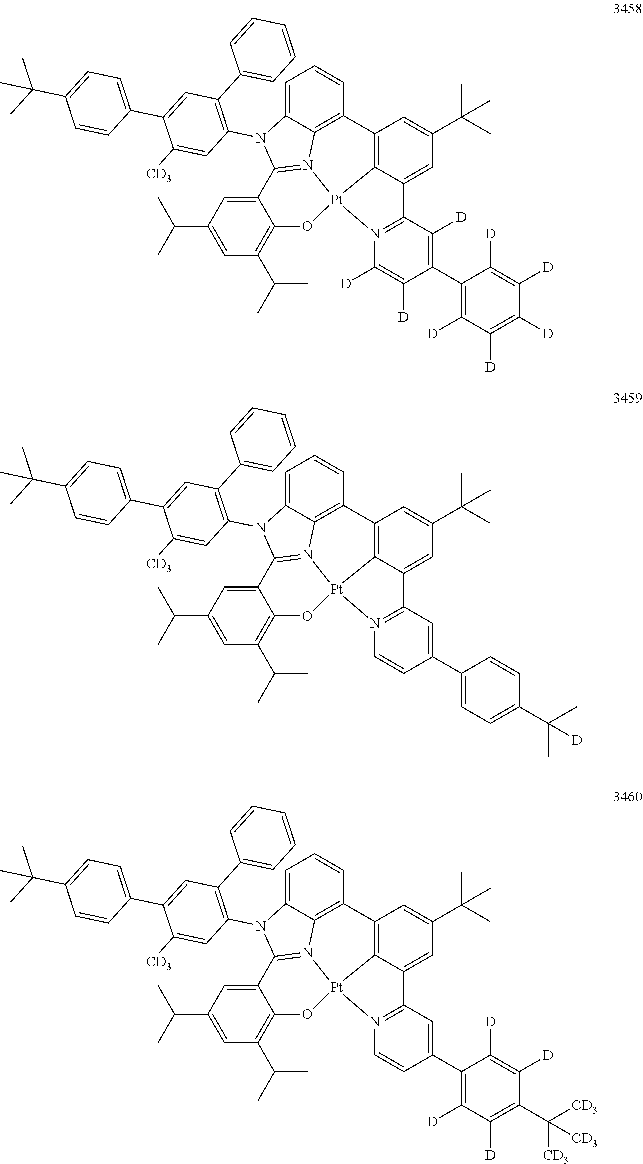

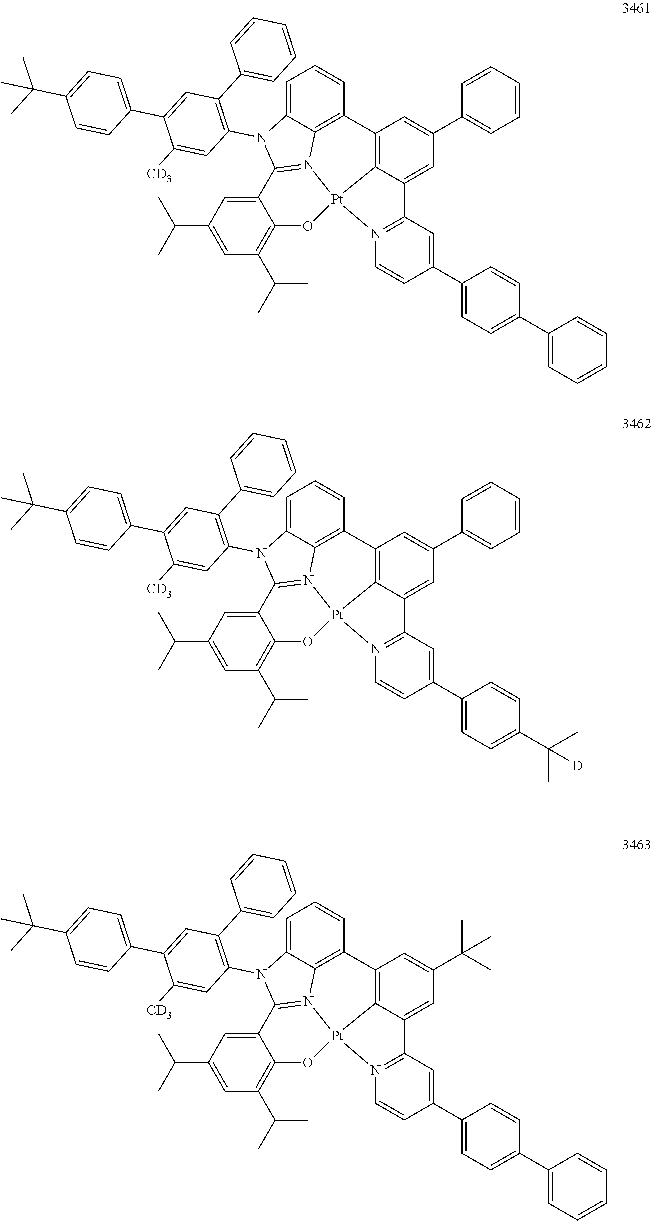

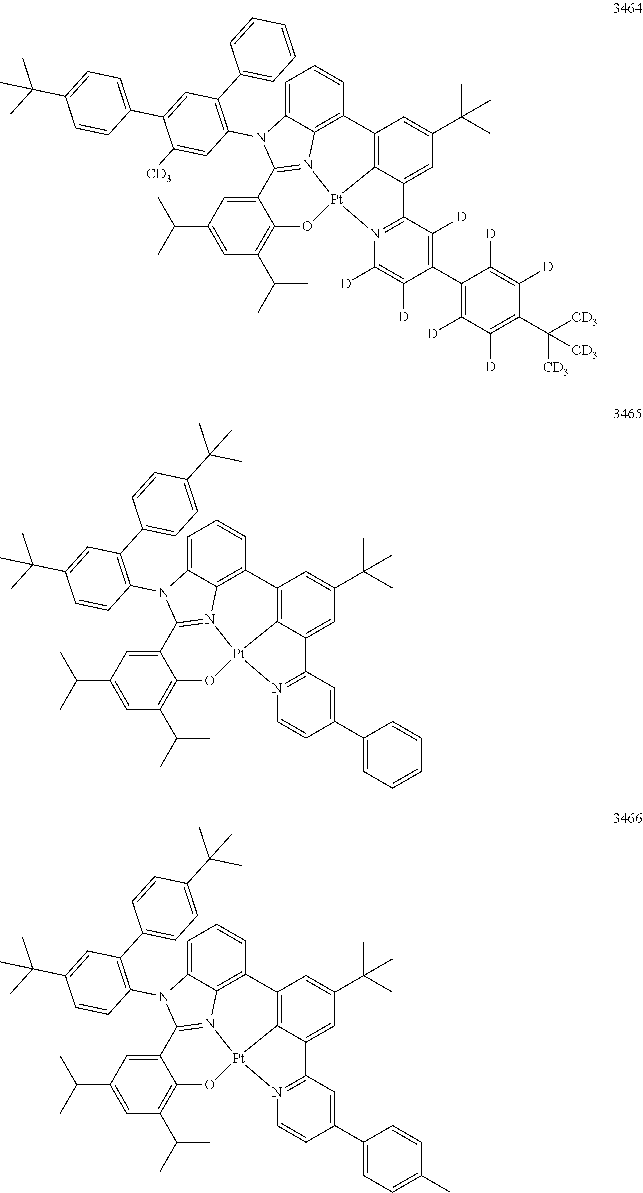

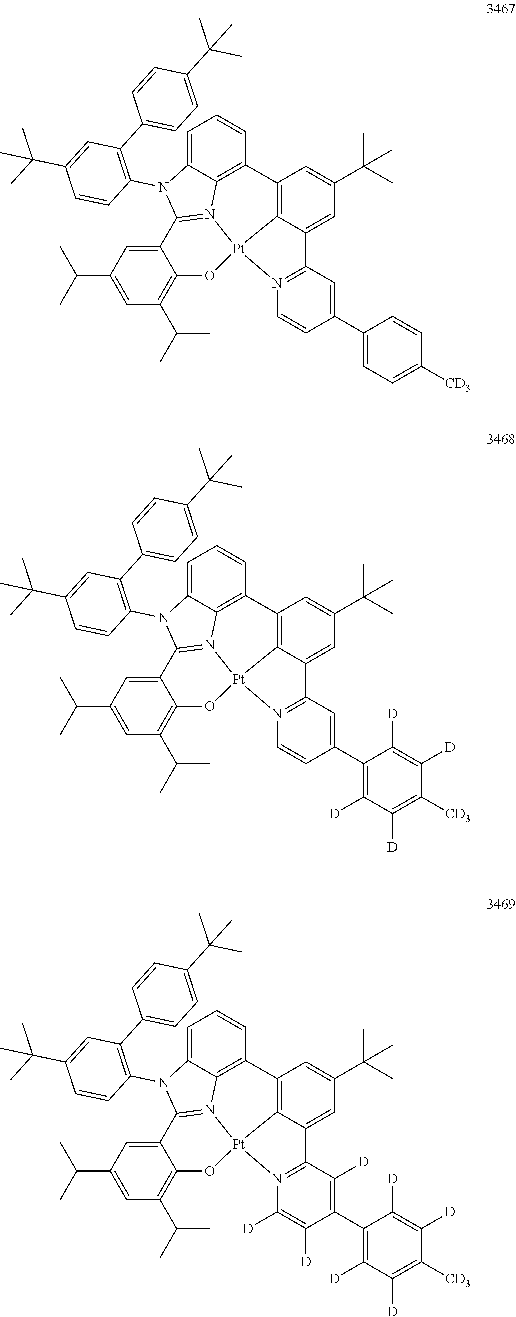

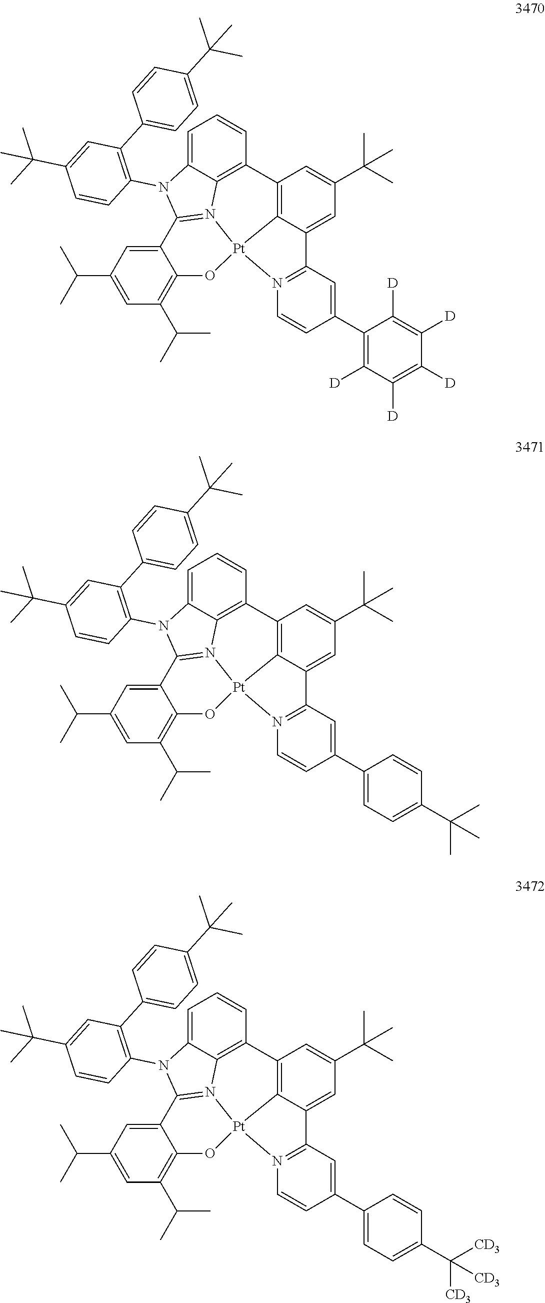

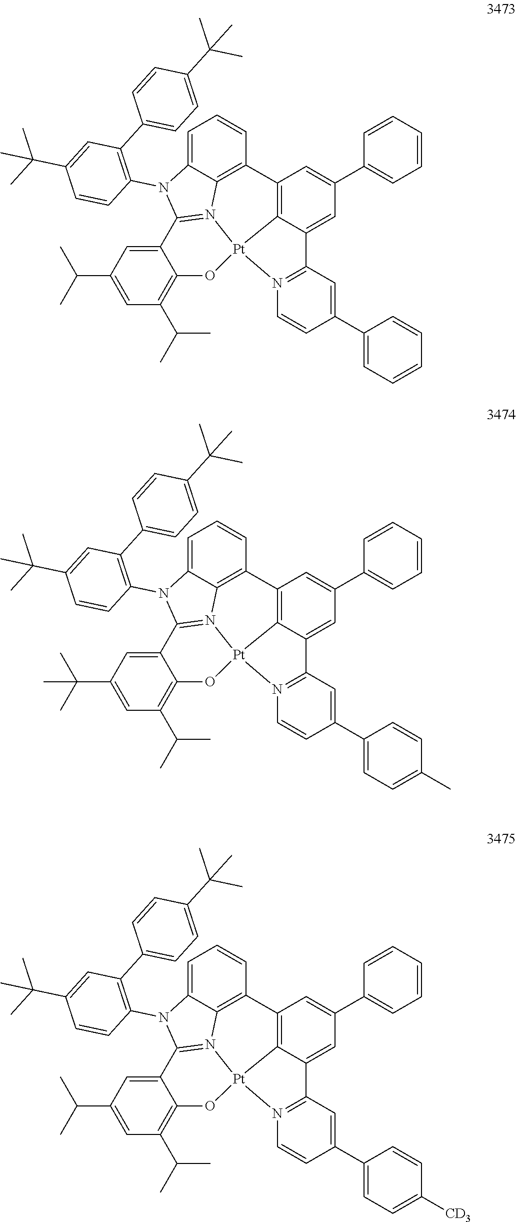

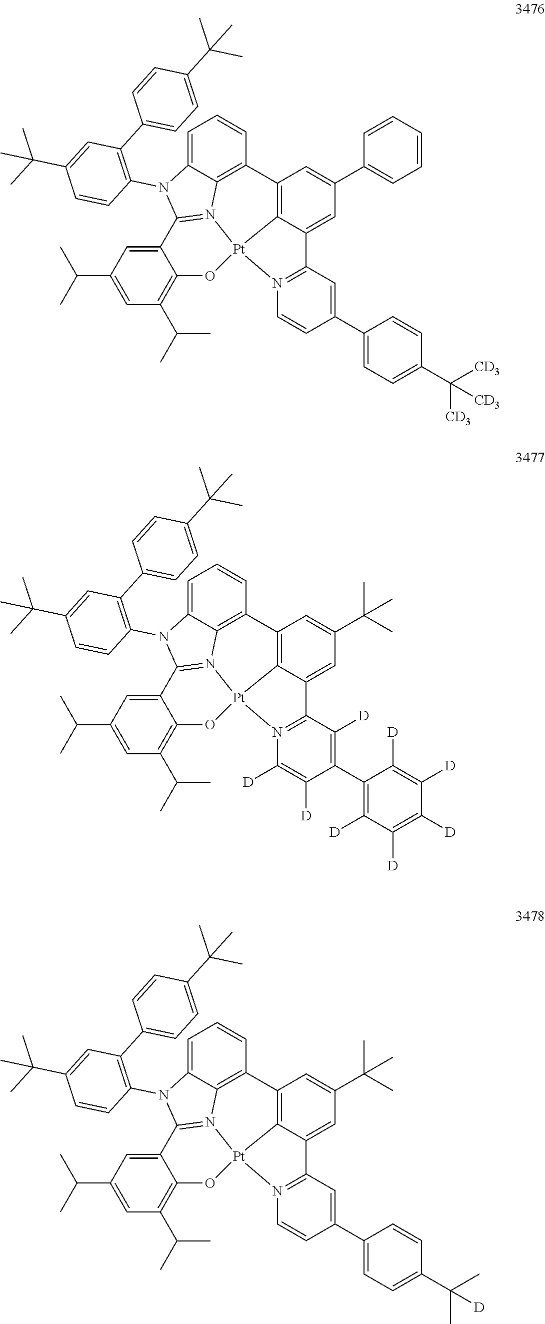

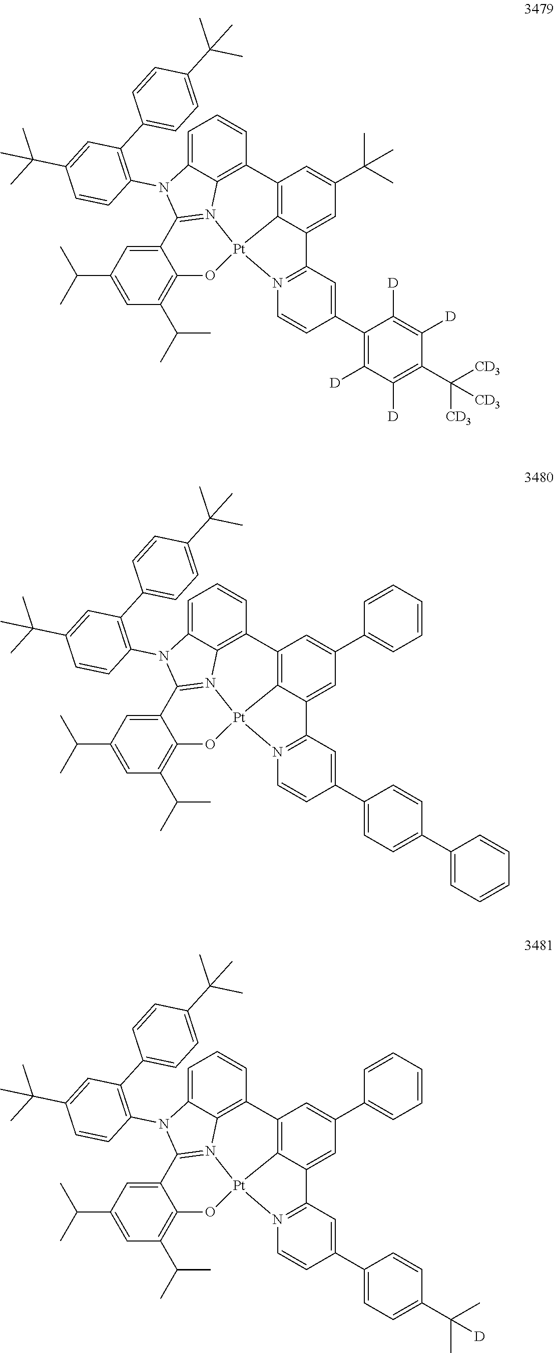

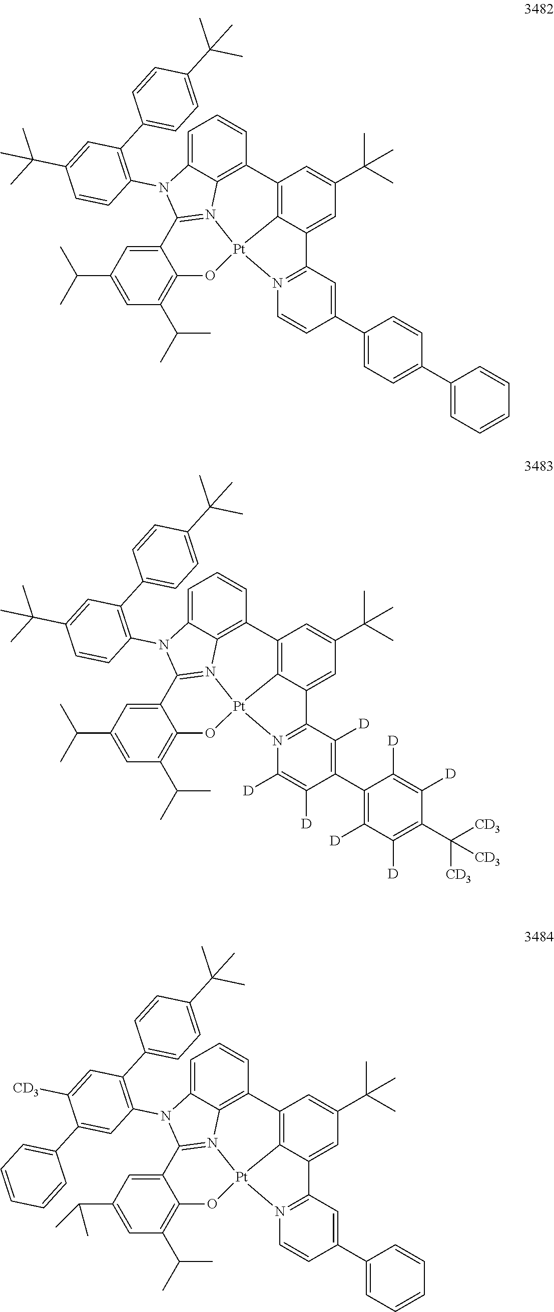

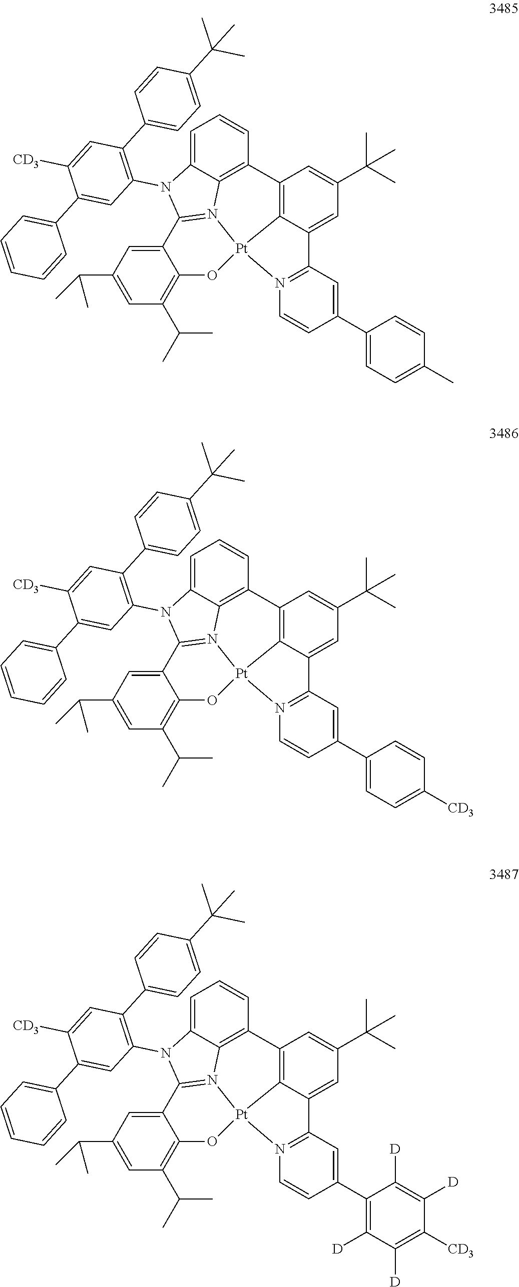

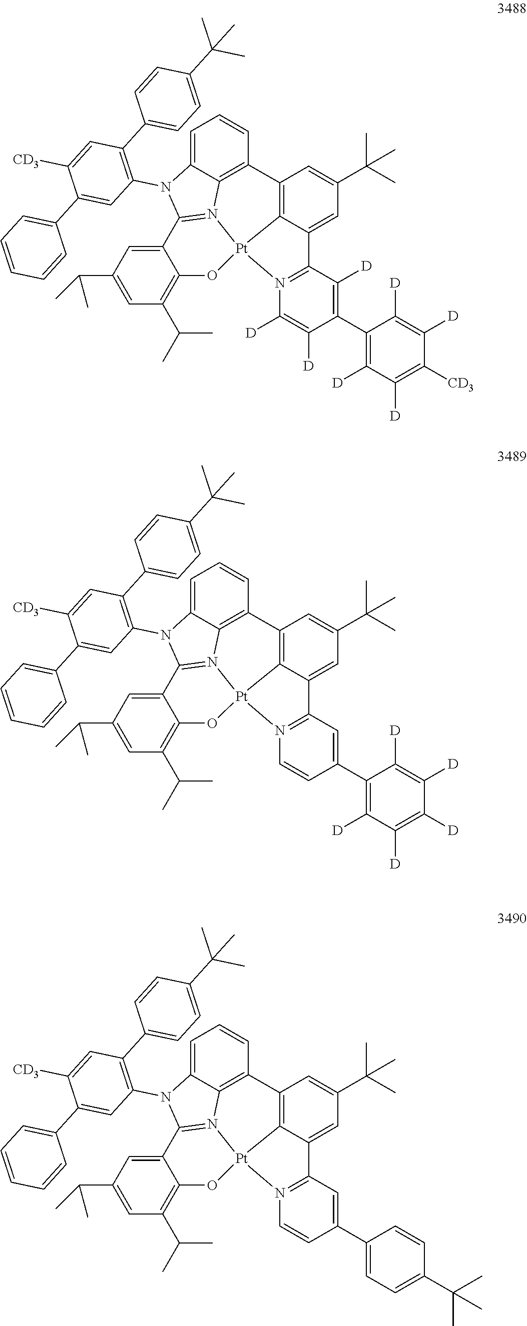

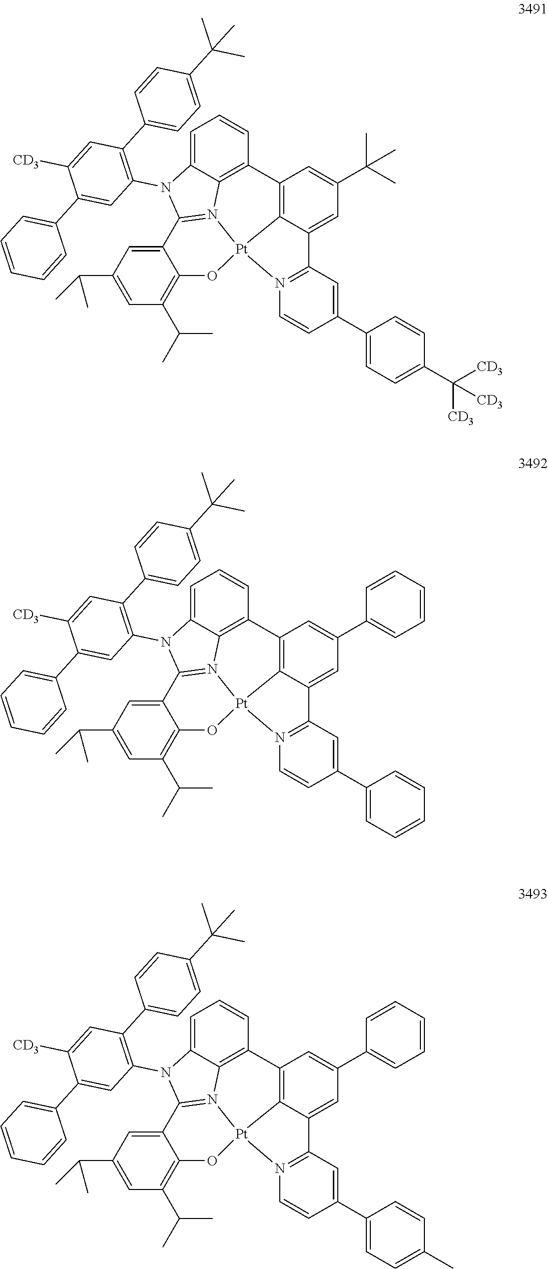

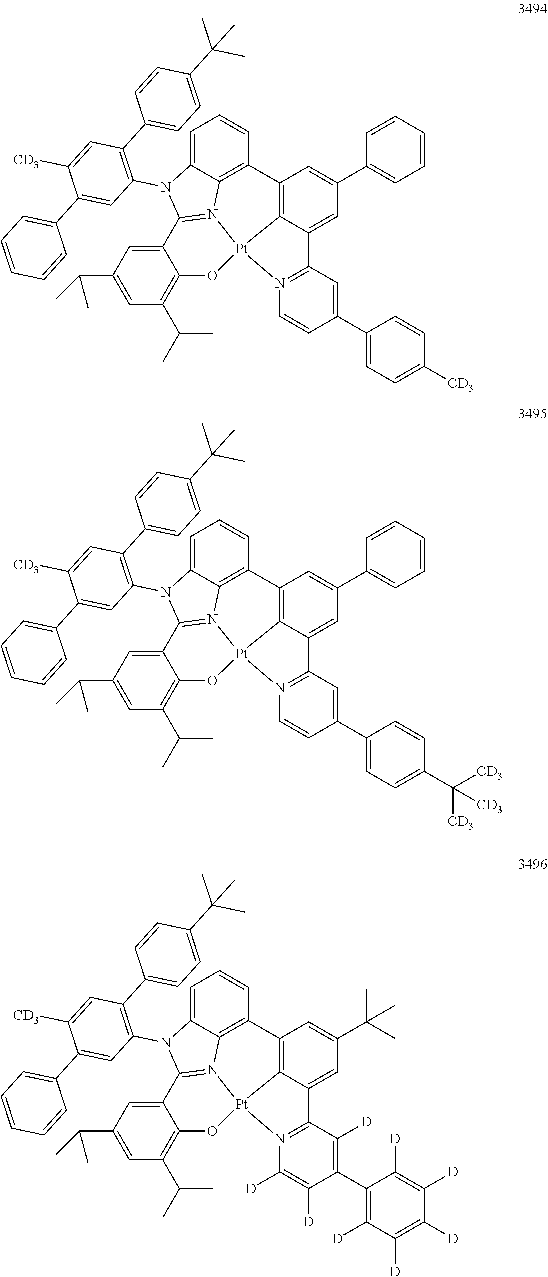

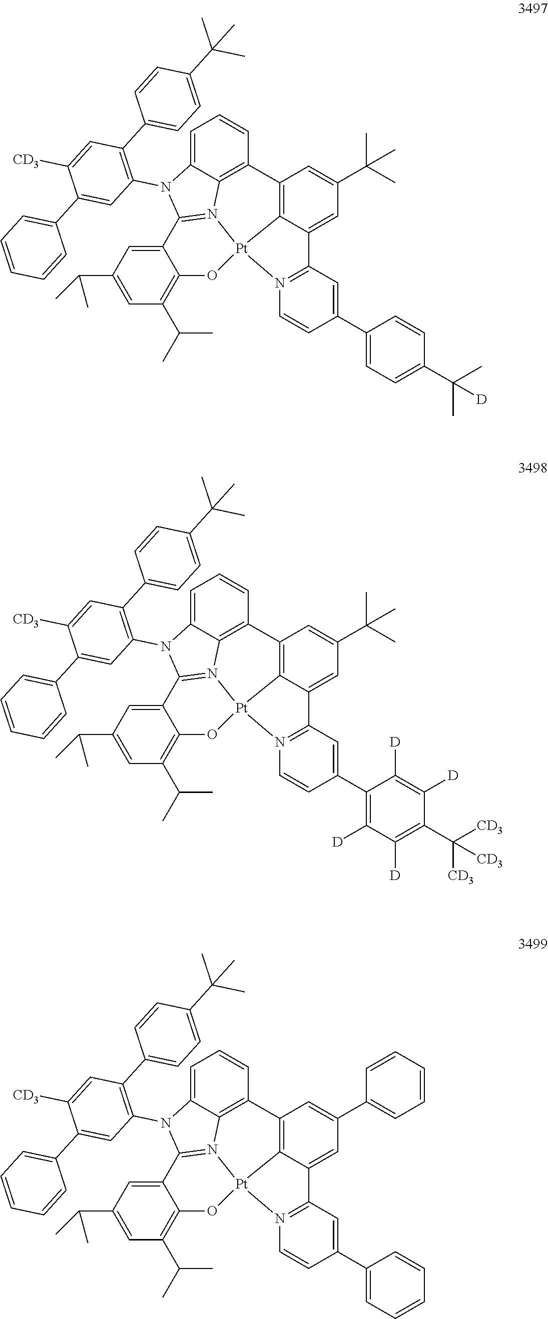

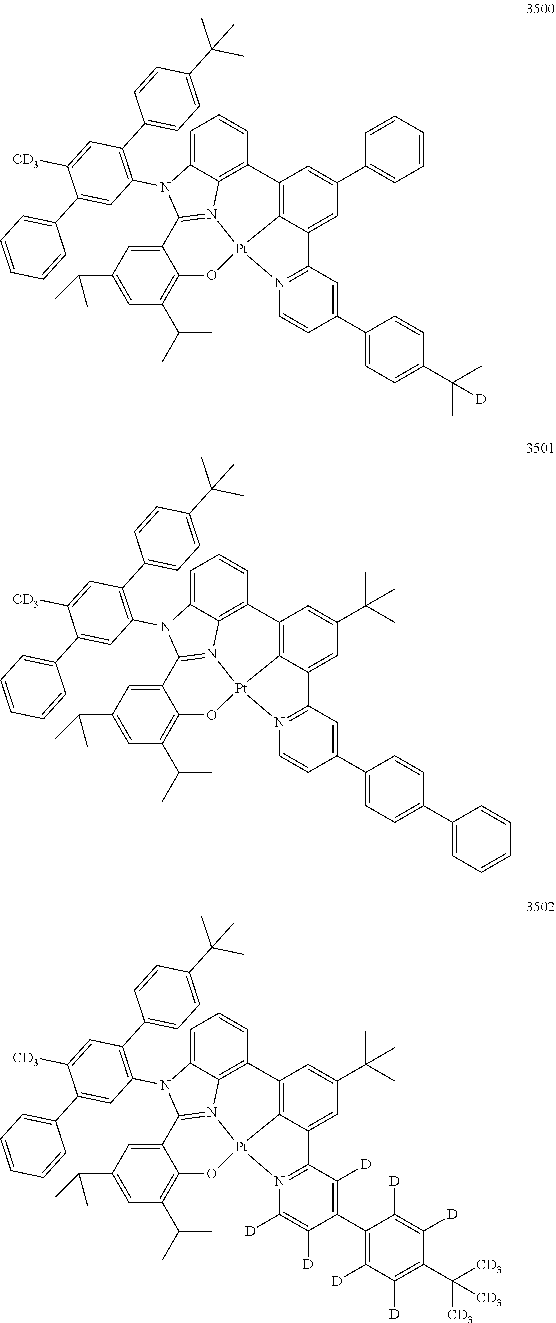

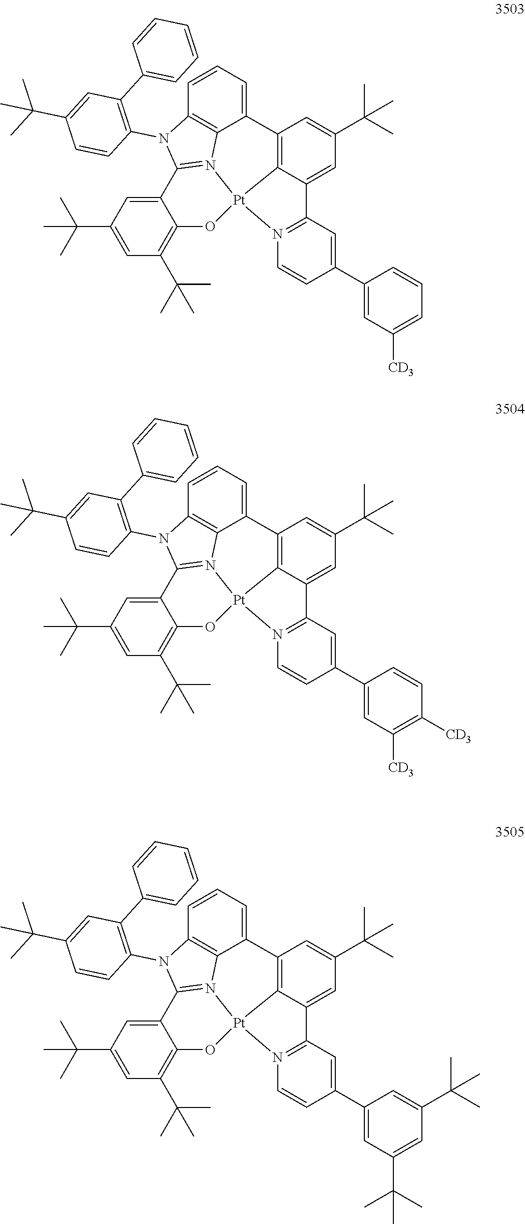

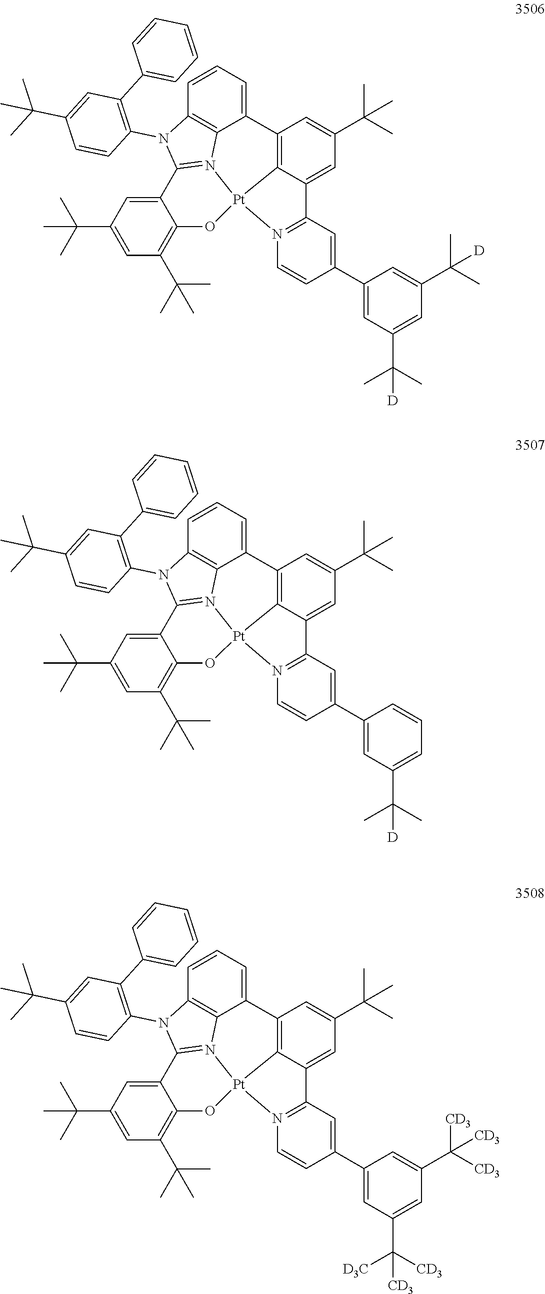

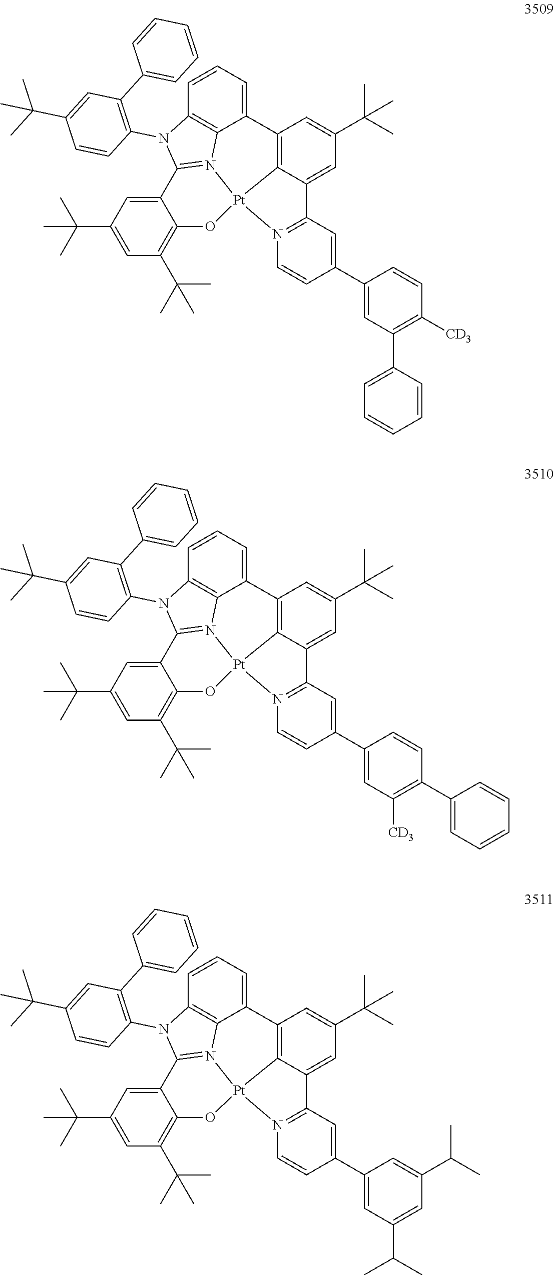

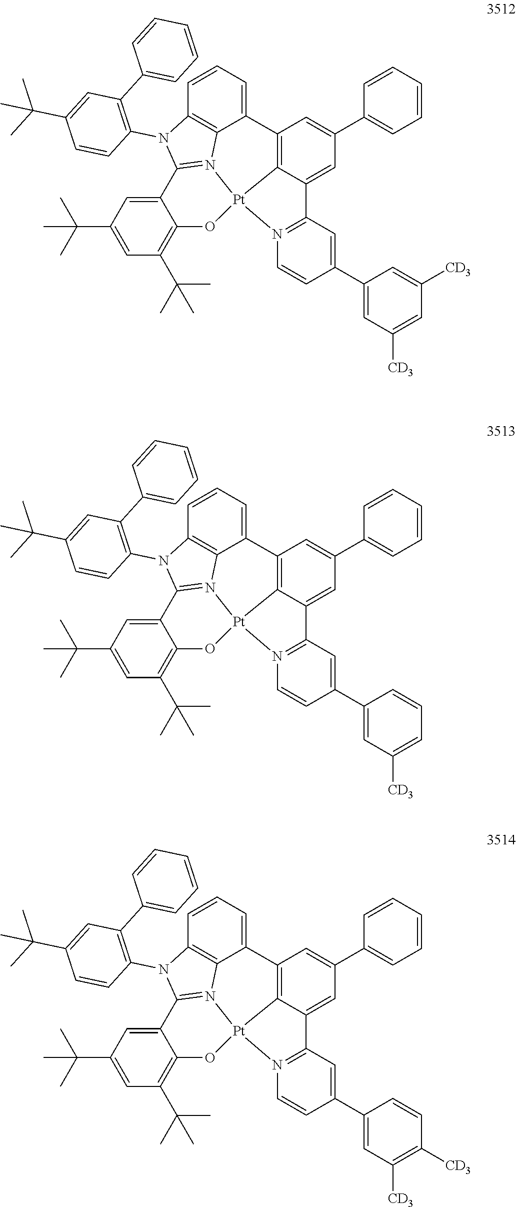

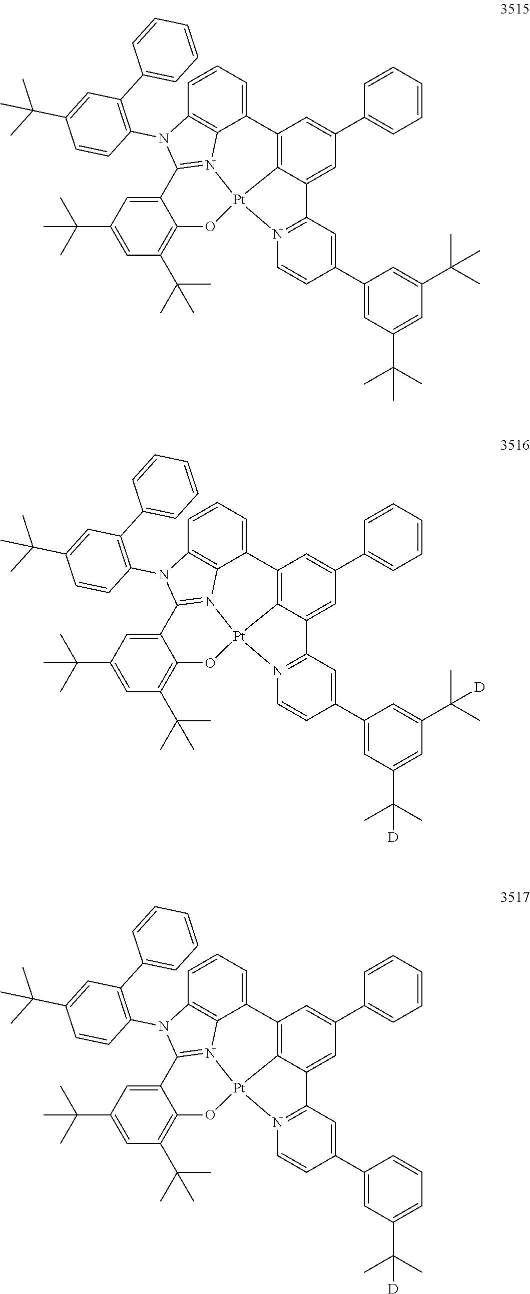

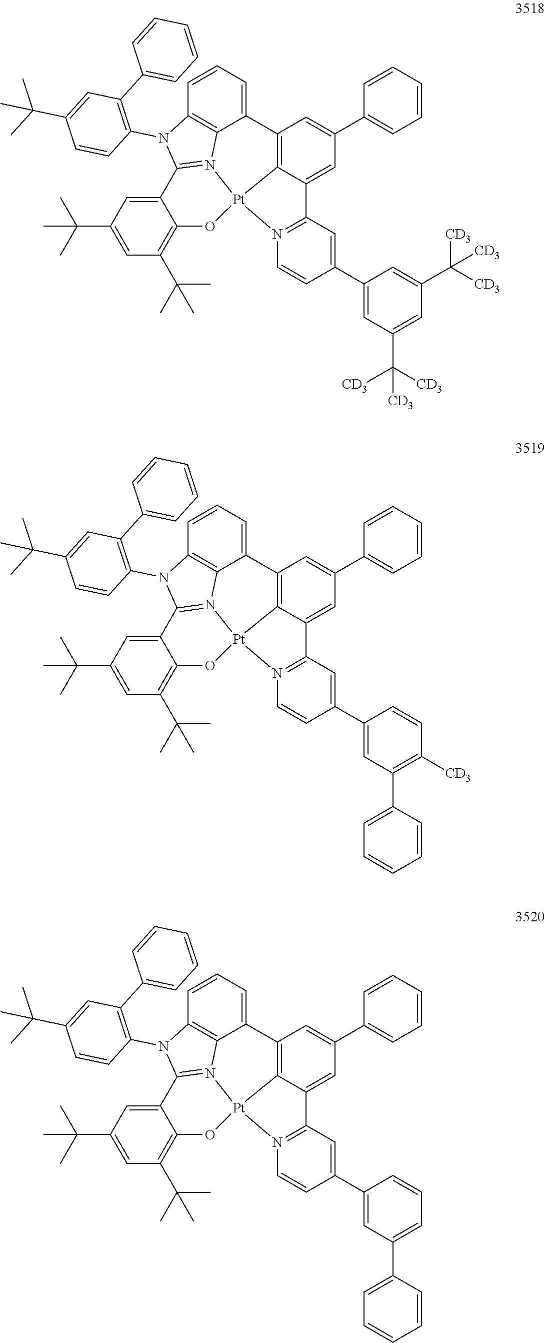

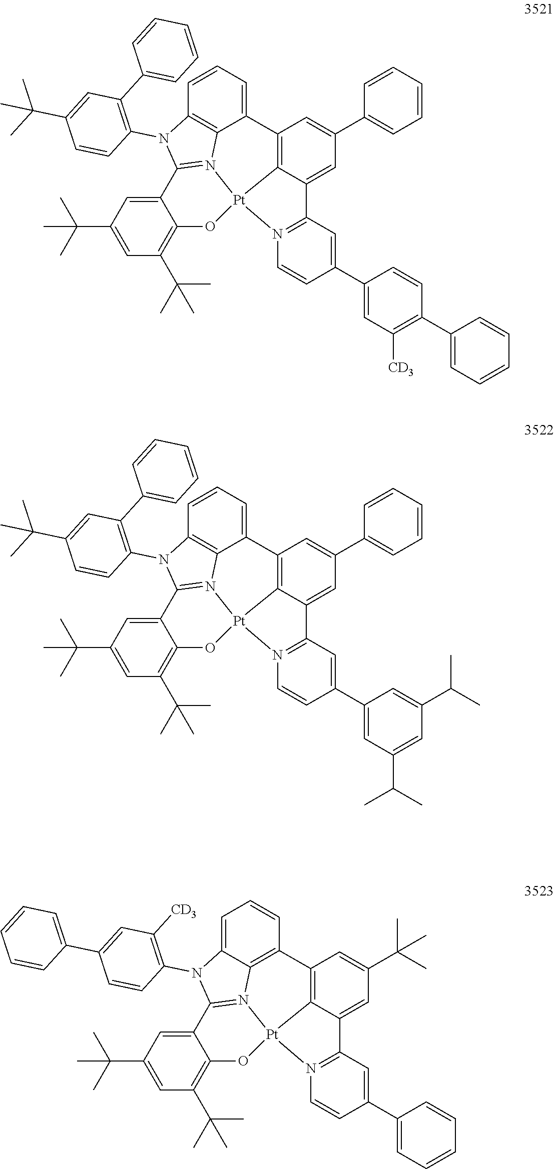

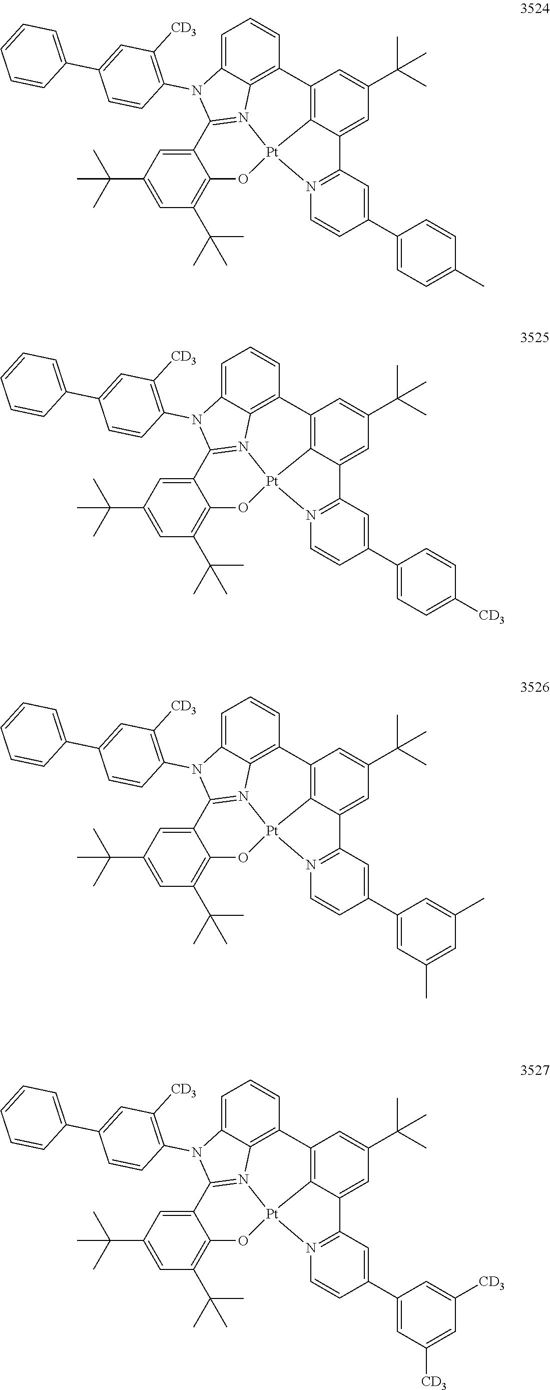

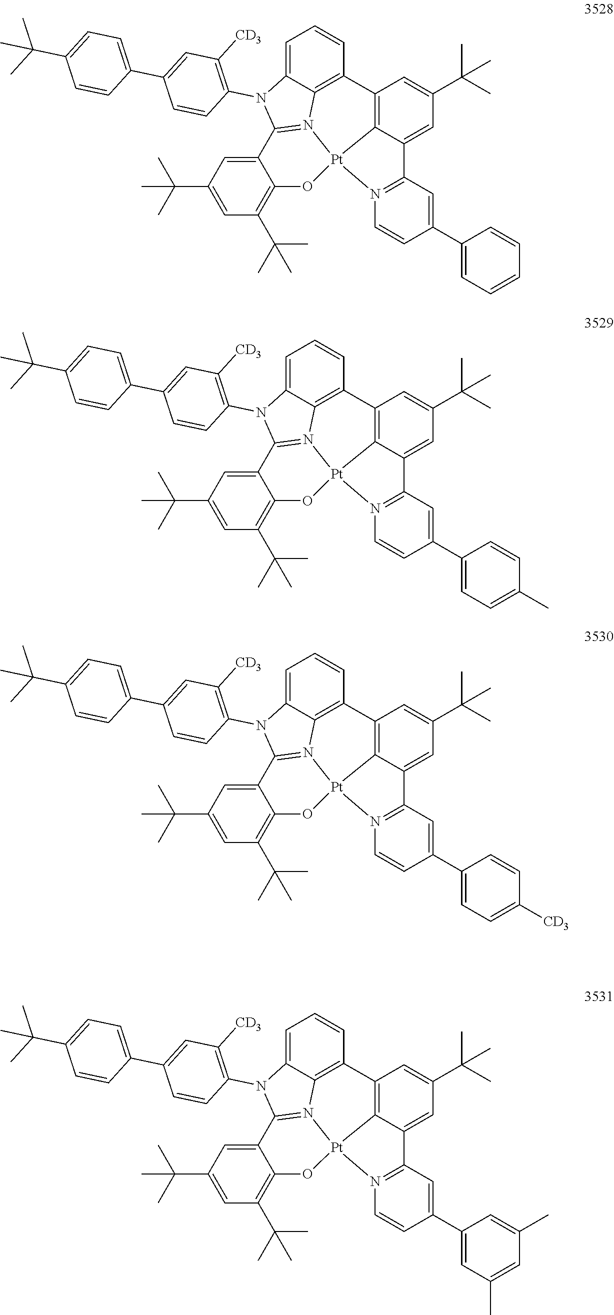

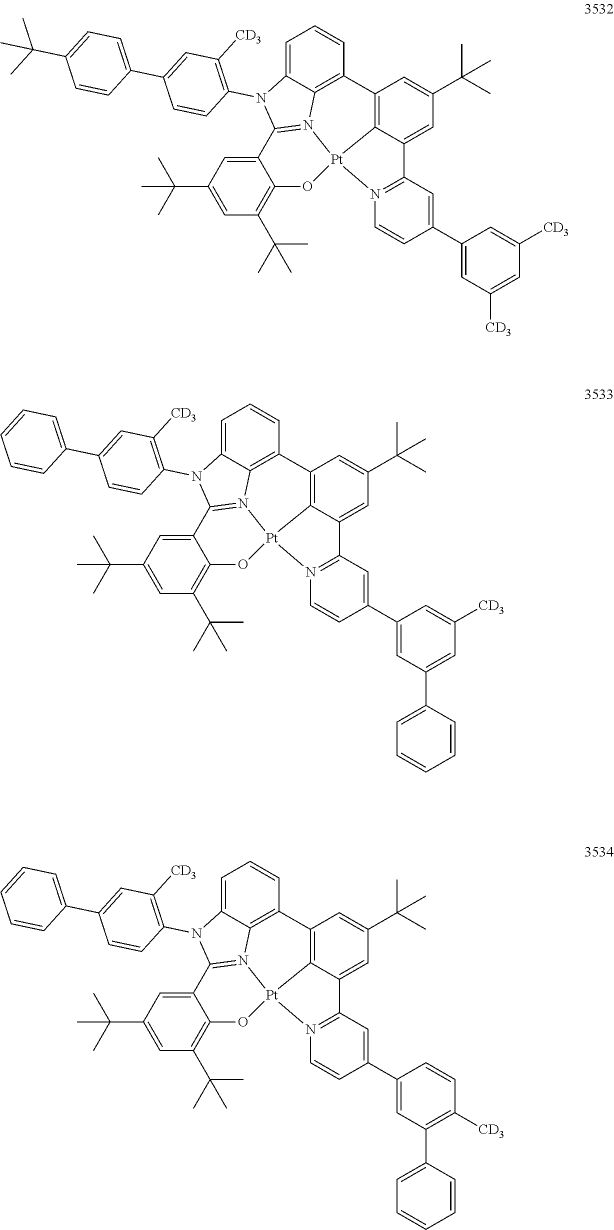

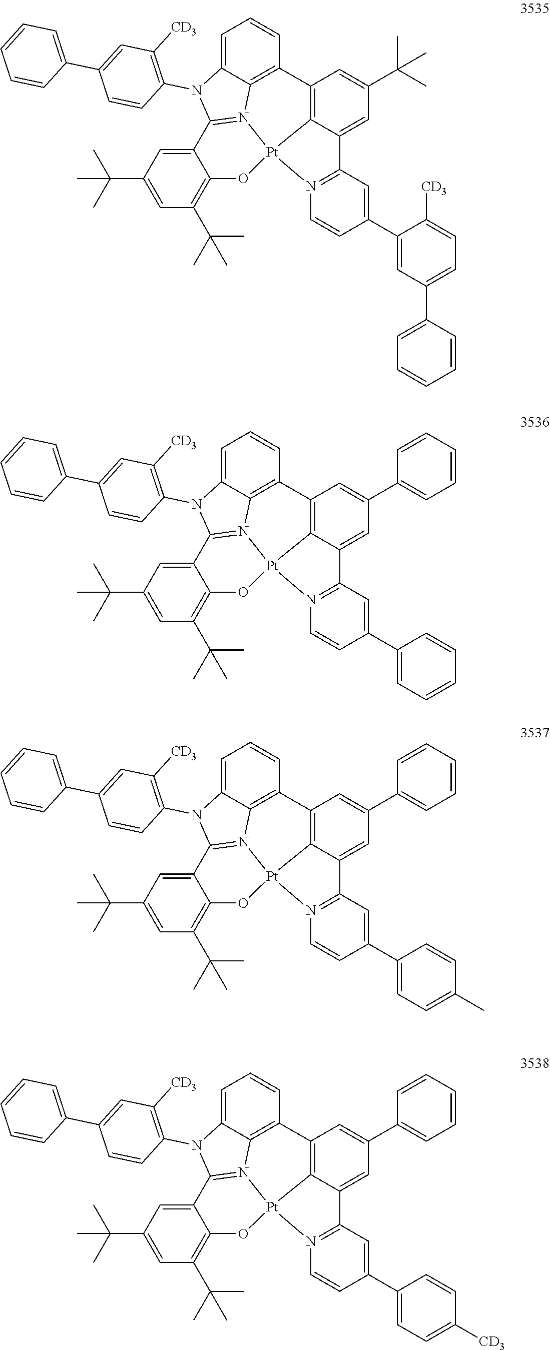

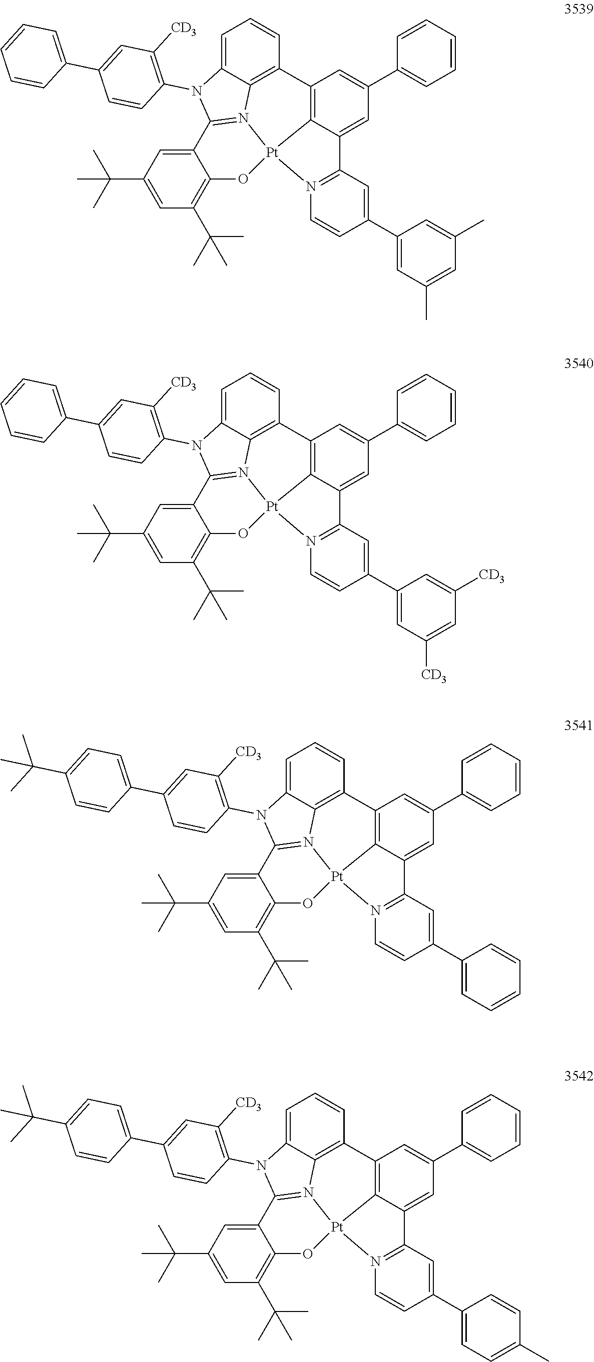

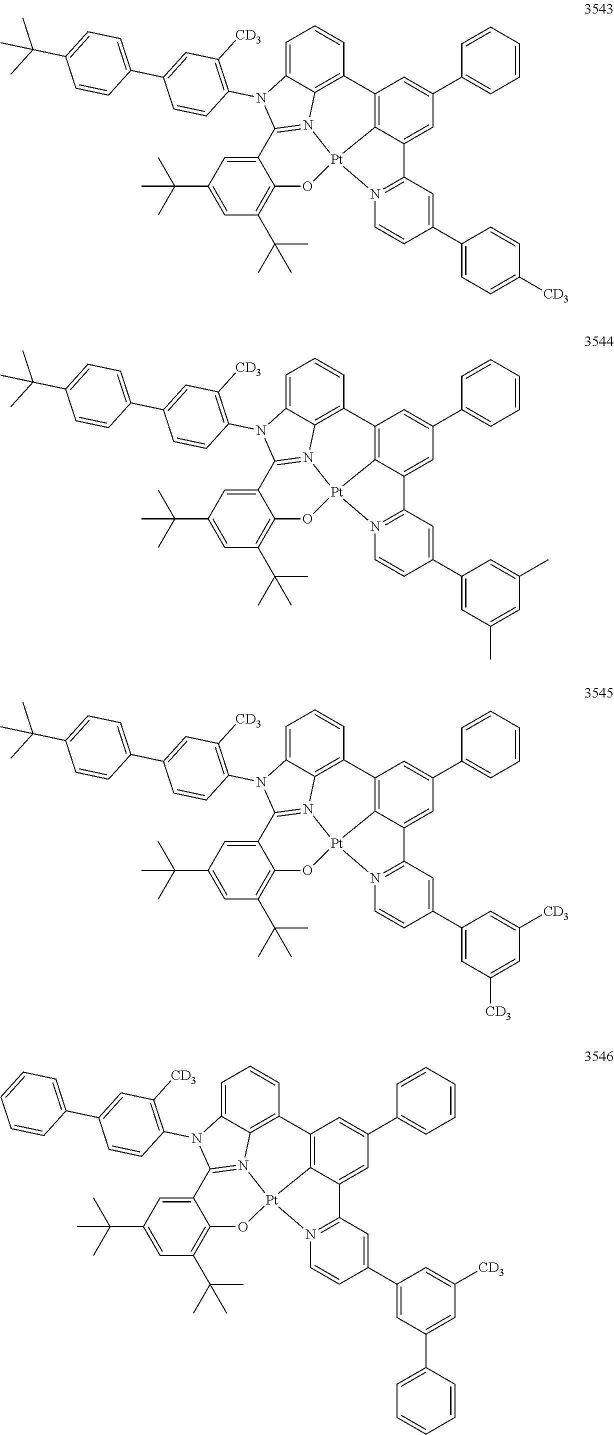

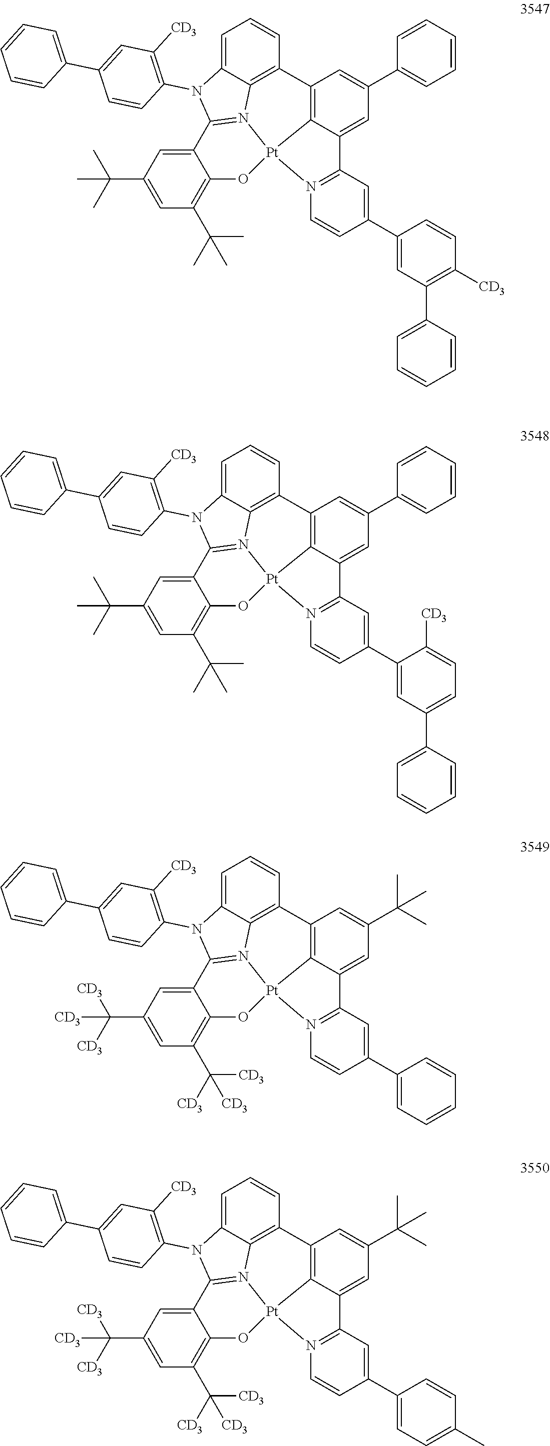

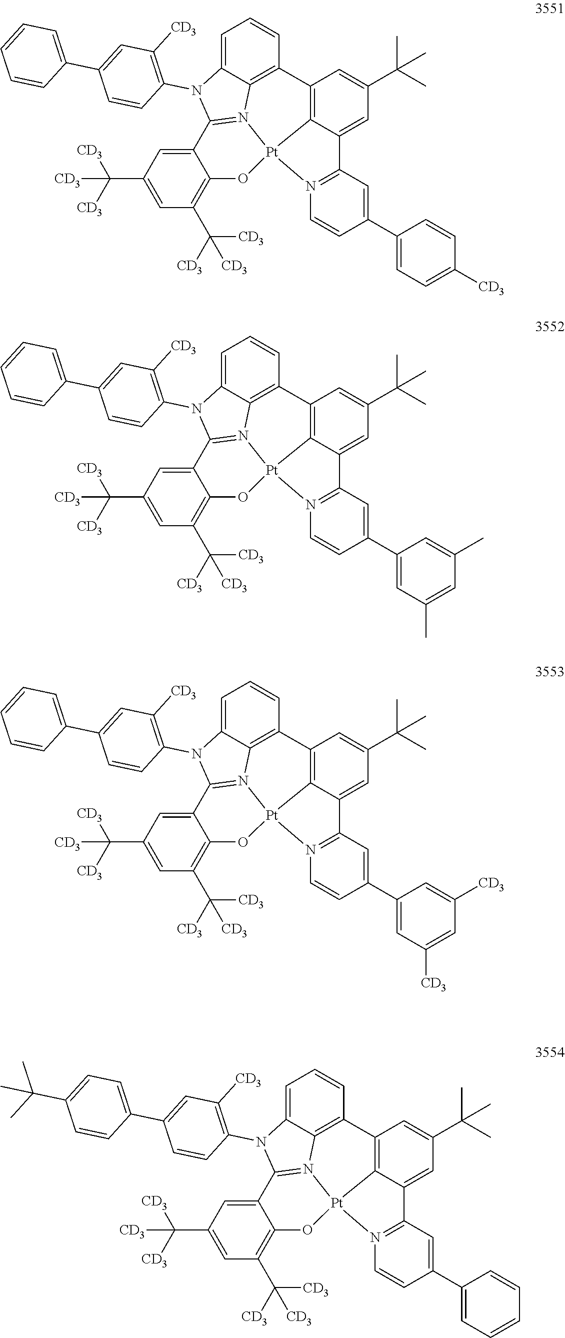

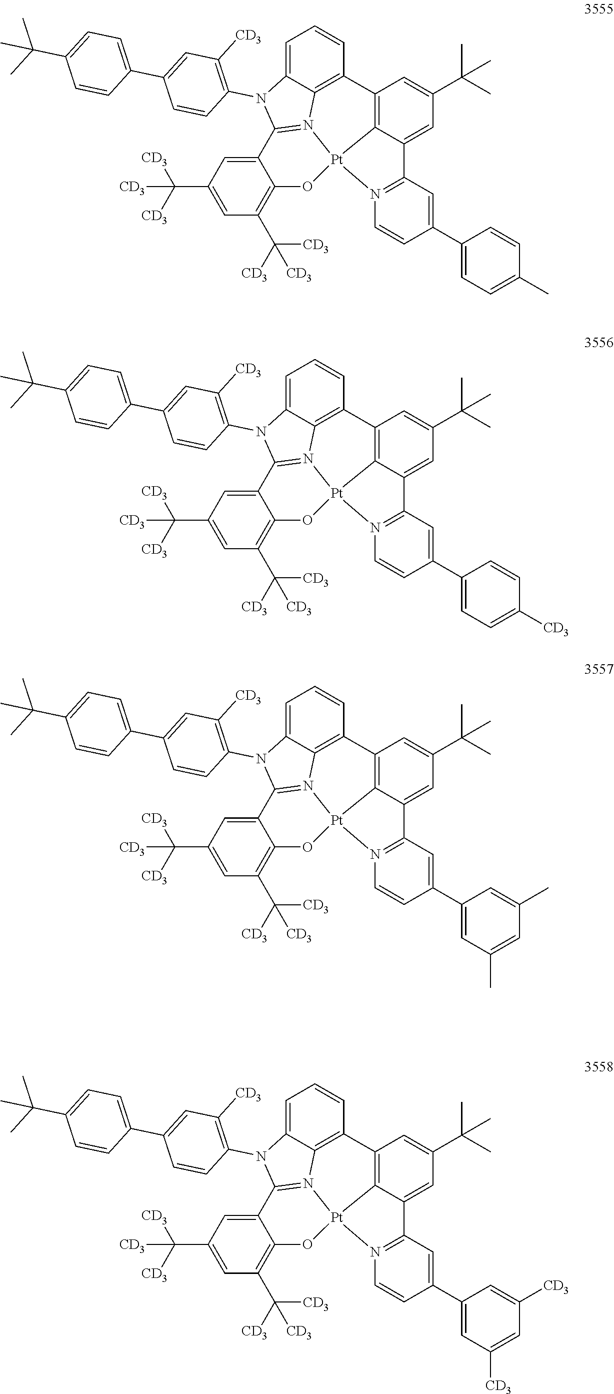

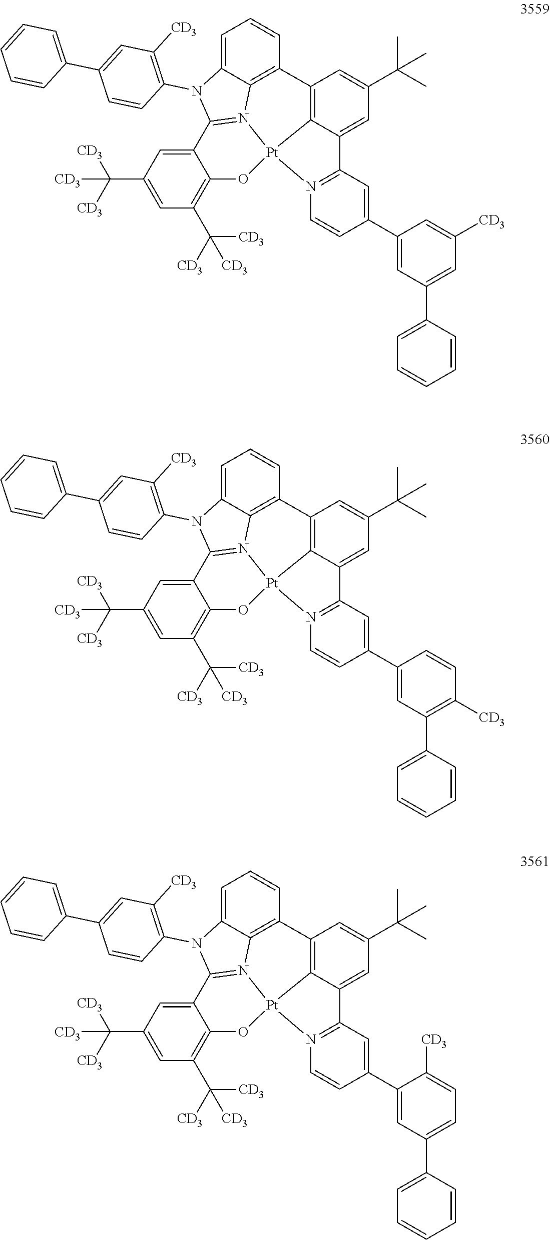

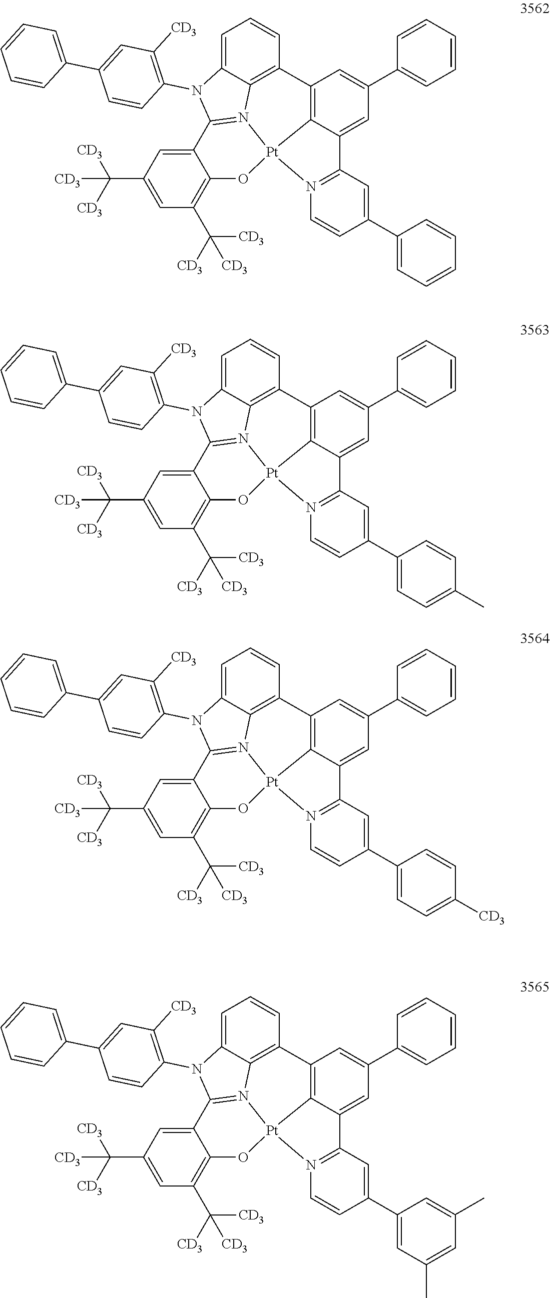

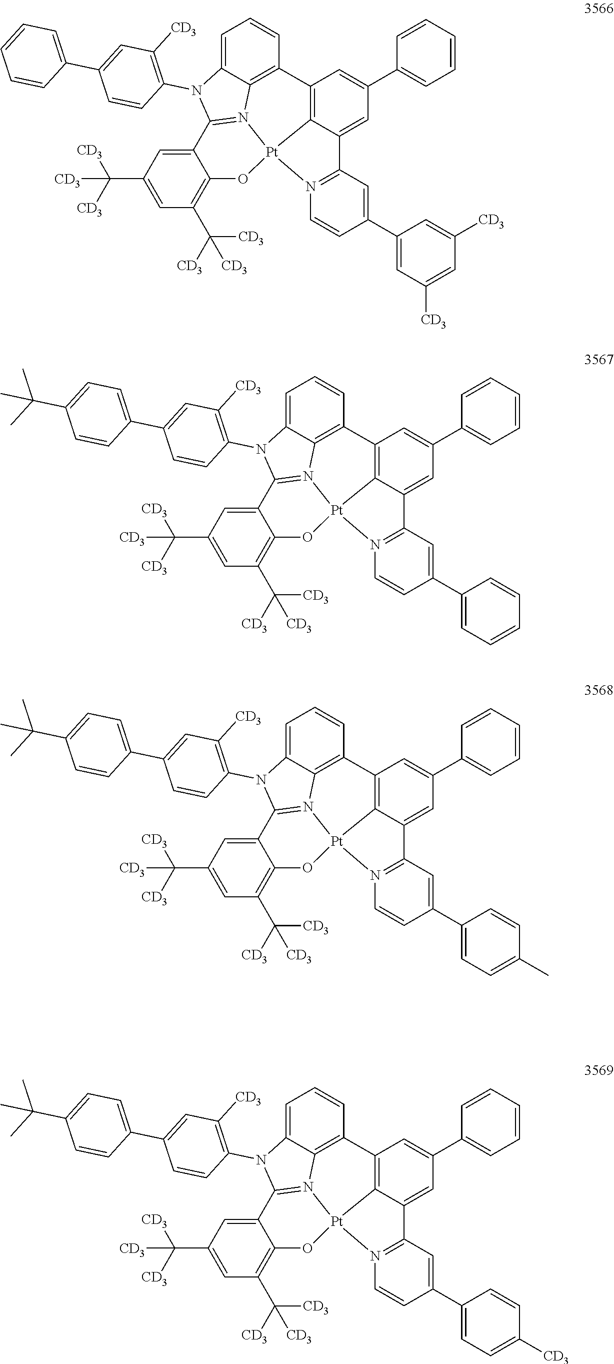

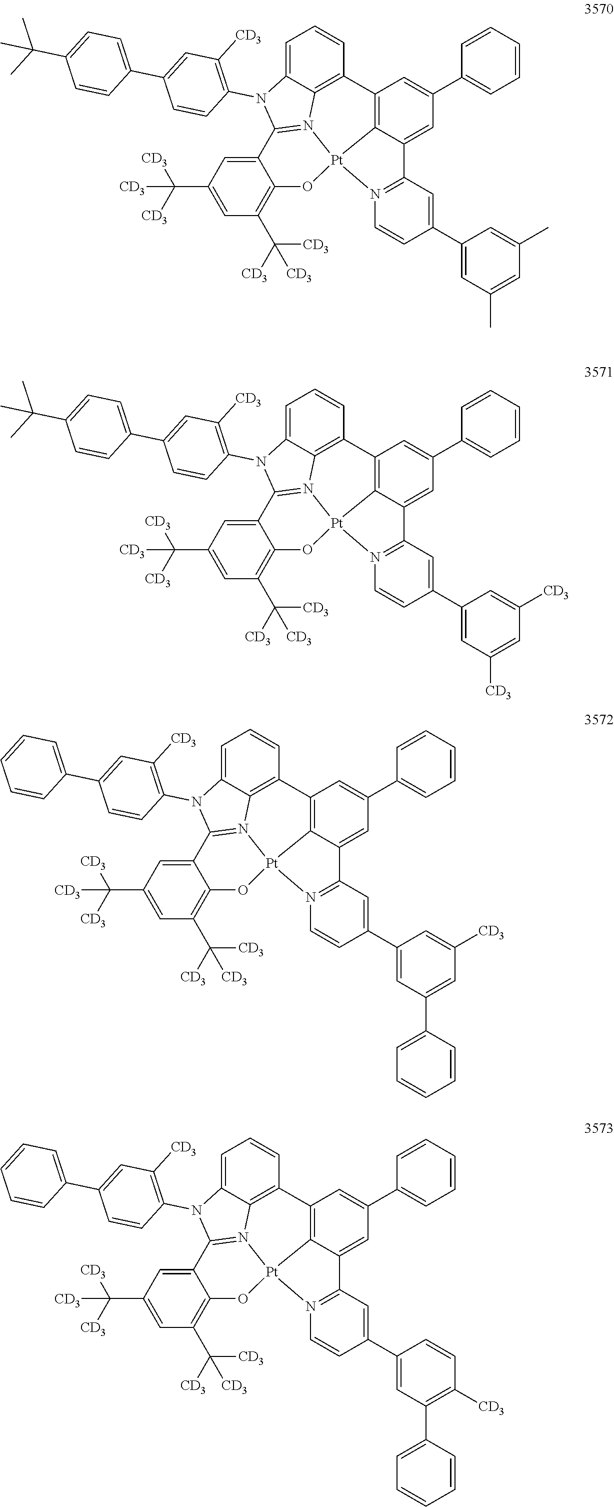

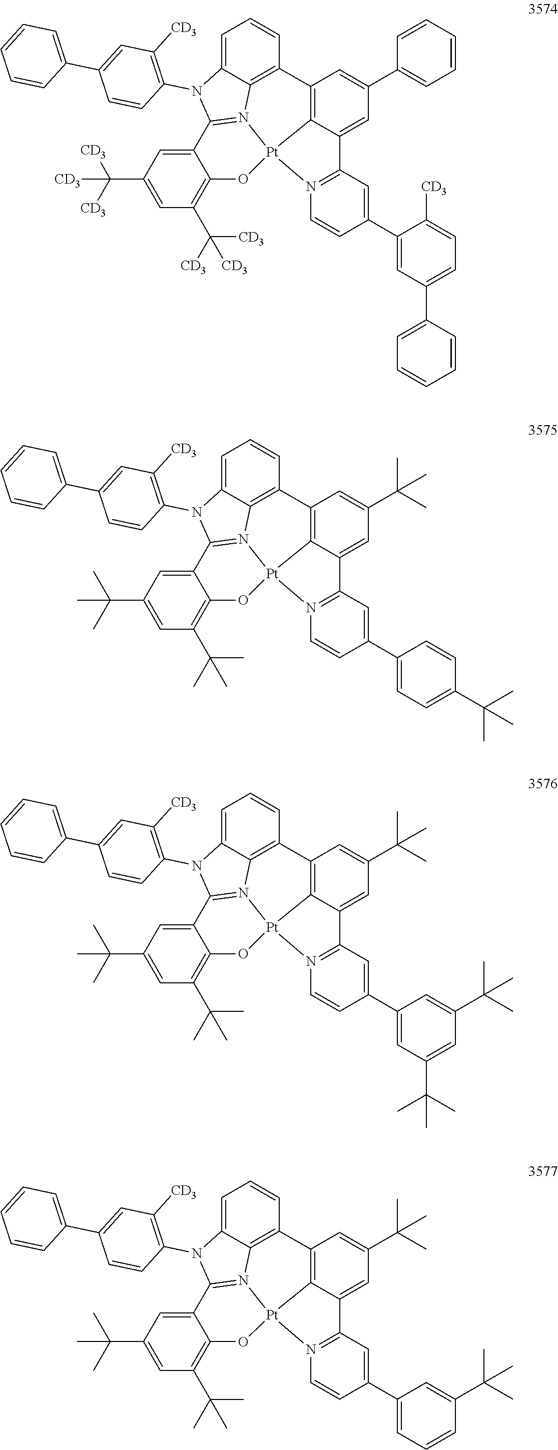

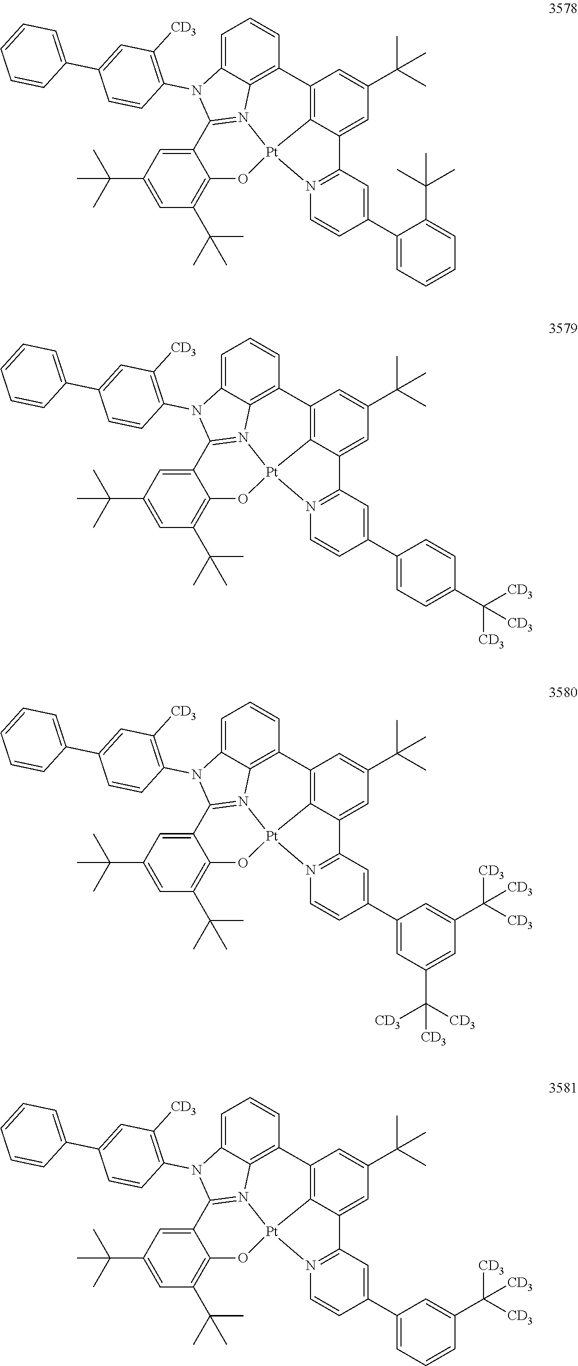

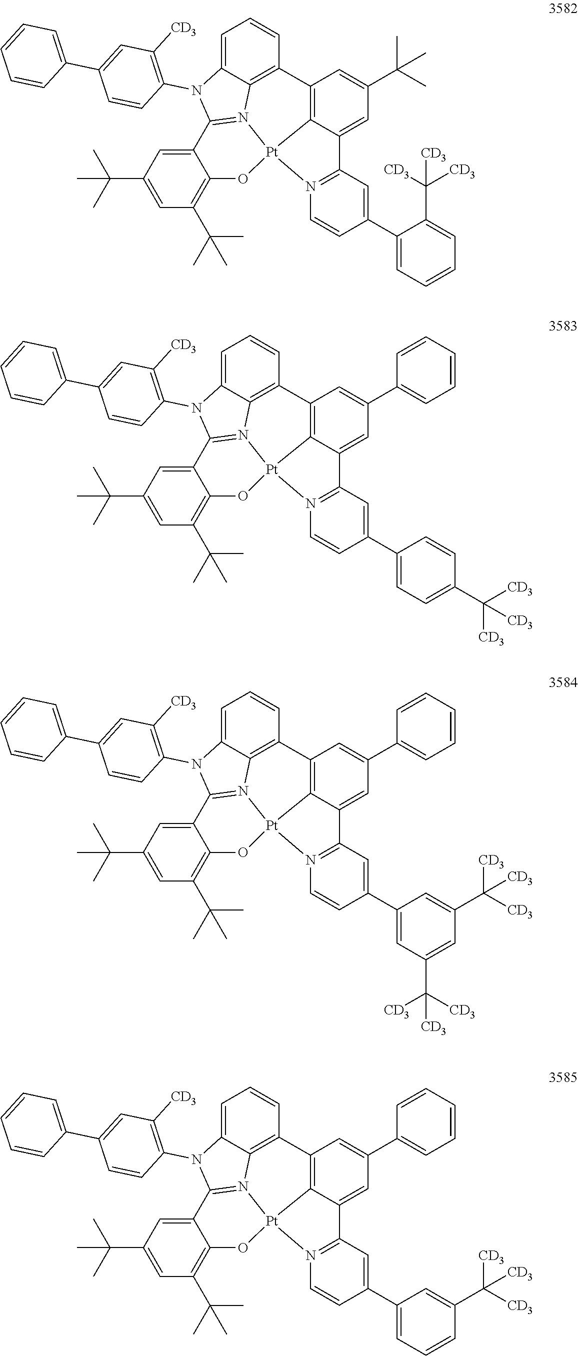

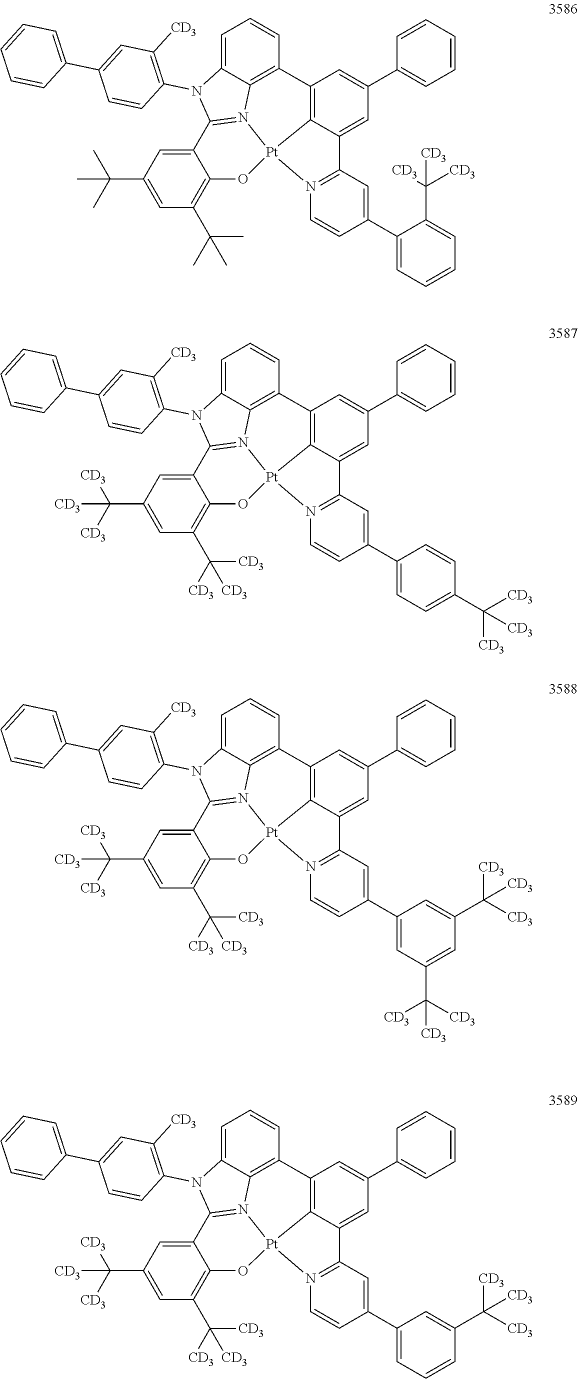

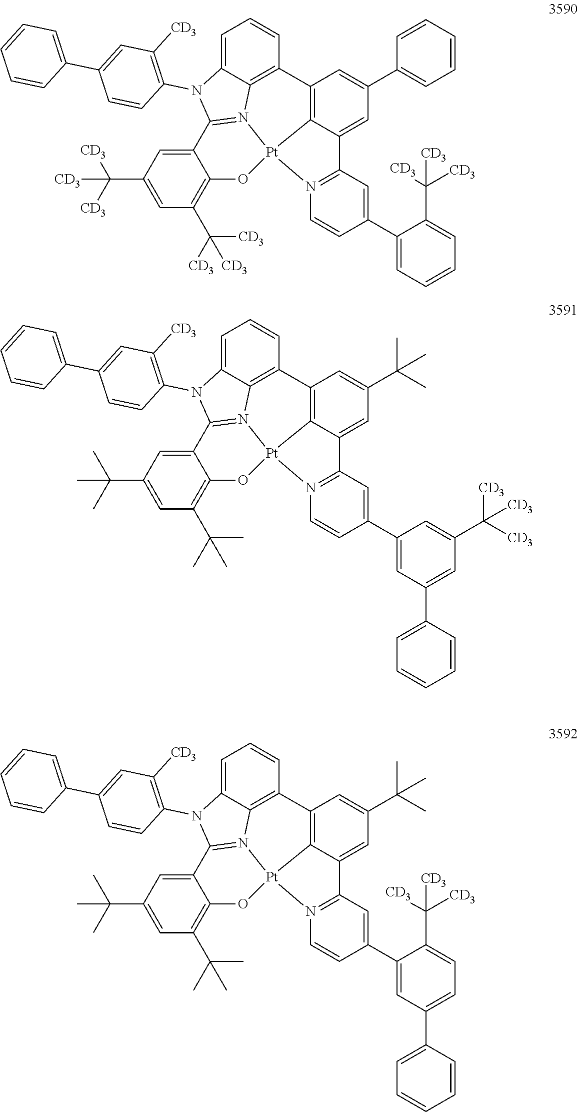

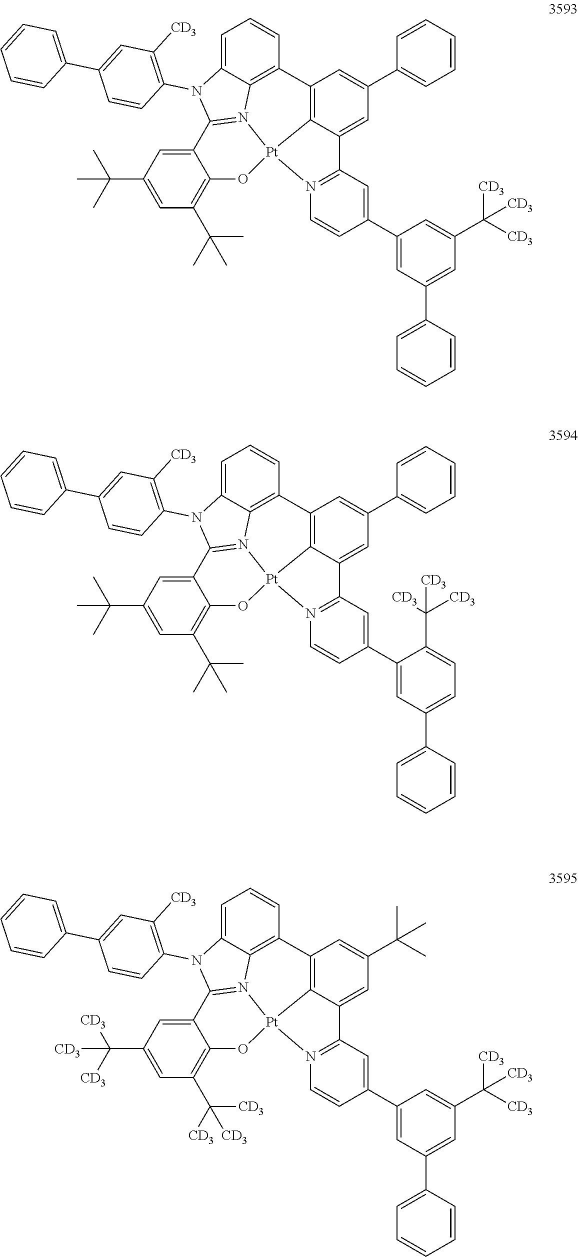

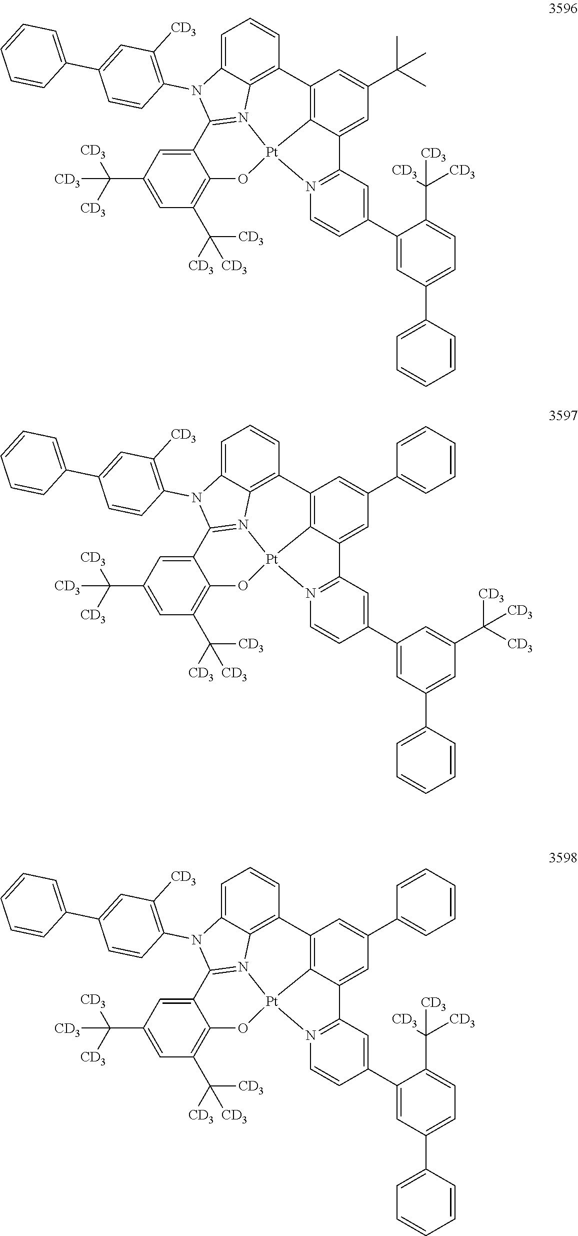

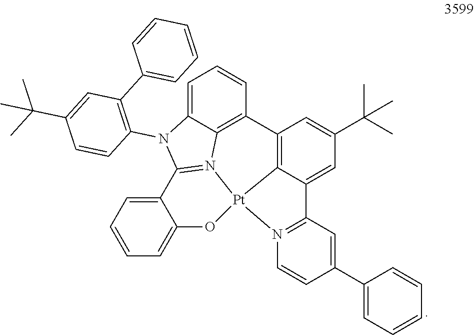

In Formula 1, A₅₁ may be a C₄-C₆₀ alkyl group, A₅₂ may be deuterium or a deuterium-containing C₁-C₆₀ alkyl group unsubstituted or substituted with a C₃-C₁₀ cycloalkyl group, a51 (the number of A₅₁) and a52 (the number of A₅₂) may each independently be an integer from 0 to 10, provided that the sum of a51 and a52 may be 1 or more. That is, in Formula 1, ring CY₅₁ is substituted with a group represented by A₅₁, a group represented by A₅₂, or any combination thereof. Since ring CY₅₁ is substituted with an electron donating group, an electronic device, for example, an organic light-emitting device, which includes the organometallic compound represented by Formula 1, may have improved luminescent efficiency and lifespan.

In an embodiment, A₅₁ in Formula 1 may be a group having the formula —C(R₅₁₁)(R₅₁₂)(R₅₁₃), wherein R₅₁₁, R₅₁₂, and R₅₁₃ are each independently a C₁-C₆₀ alkyl group, for example, a C₁-C₅₀ alkyl group, a C₁-C₄₀ alkyl group, a C₁-C₃₀ alkyl group, a C₁-C₂₀ alkyl group, a C₁-C₁₀ alkyl group, or a C₁-C₅ alkyl group. While not wishing to be bound by theory, it is understood that when A₅₁ has the formula —C(R₅₁₁)(R₅₁₂)(R₅₁₃), the organometallic compound represented by Formula 1, may have improved luminescent efficiency and lifespan compared to the compounds in which at least one of R₅₁₁, R₅₁₂, or R₅₁₃ is hydrogen.

In an embodiment, A₅₂ in Formula 1 may be a group having the formula —CD(R₅₂₁)(R₅₂₂), wherein R₅₂₁ and R₅₂₂ are each independently a C₁-C₆₀ alkyl group, a C₁-C₆₀ alkyl group substituted with deuterium, a C₃-C₁₀ cycloalkyl group, or any combination thereof. In an embodiment, A₅₂ in Formula 1 may be a group having the formula —CD₂(R₅₂₁), wherein R₅₂₁ is the same as defined above. In still another embodiment, A₅₂ may be a fully deuterated group, such as CD₃, C₂D₅, C₃D₇, C₄D₉, but is not limited thereto. While not wishing to be bound by theory, it is understood that when A₅₂ has the formula —CD(R₅₂₁)(R₅₂₂), the formula —CD₂(R₅₂₁), or wherein A₅₂ is a fully deuterated group, the organometallic compound represented by Formula 1, may have improved luminescent efficiency and lifespan compared to the compounds in which at least one of R₅₂₁ or R₅₂₂ is hydrogen.

A₅₂ may be a partially or fully deuterated C₁-C₆₀ alkyl group unsubstituted or substituted with a C₃-C₁₀ cycloalkyl group. When the degree of deuteration is 100%, the group A₅₂ is fully deuterated. When the degree of deuteration is lower than 100%, the group A₅₂ is partially deuterated. The degree of deuteration of the group A₅₂ may be calculated by using Equation 10:

degree of deuteration (%)=n_{D2}/(n_{H2}+n_{D2})×100. Equation 10

In Equation 10, n_{H2} represents the total number of hydrogens included in the group A₅₂, and

n_{D2} represents the total number of deuterium atoms included in the group A₅₂.

In an embodiment, the degree of deuteration of the group A₅₂ may be about 5% or more, about 10% or more, about 20% or more, about 30% or more, about 40% or more, about 50% or more, about 60% or more, about 70% or more, about 90% or more, about 95% or more, about 96% or more, about 97% or more, about 98% or more, or about 99% or more, but embodiments of the present disclosure are not limited thereto.

In addition, a53 that is the number of groups represented by

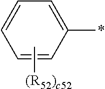

in Formula 1 may be an integer from 1 to 10. That is, since a53 in Formula 1 is not 0, ring CY₅₁ in Formula 1 is substituted with a group represented by

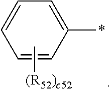

Therefore, due to the resonance effect caused by the group represented by

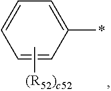

an electronic device, for example, an organic light-emitting device, which includes the organometallic compound represented by Formula 1, may have improved luminescent efficiency and lifespan. In addition, although not limited by a specific theory, a group represented by

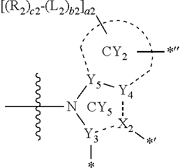

in Formula 1 is protected from electrons, heat, or the like by the group represented by

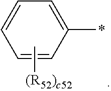

Therefore, an electronic device, for example, an organic light-emitting device, which includes the organometallic compound represented by Formula 1, may have improved luminescent efficiency and lifespan.

In Formula 1, the sum of a51 and a52 may be 1 or more and a53 may be an integer from 1 to 10. Thus, in an embodiment, the organometallic compound may simultaneously include

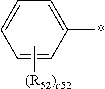

and A₅₁. In another embodiment, the organometallic compound may simultaneously include

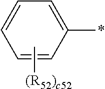

### and A₅₂.

For example, highest occupied molecular orbital (HOMO), lowest unoccupied molecular orbital (LUMO), energy band gap, singlet (Si), and triplet (Ti) energy levels of Compounds 1 to 8 were evaluated by using a DFT method of Gaussian program (structurally optimized at a level of B3LYP, 6-31G(d,p)). Evaluation results are shown in Table 1 below.

TABLE 1

| | | | Energy | | |
|---|---|---|---|---|---|
| Compound | HOMO | LUMO | band gap | S₁ | T₁ |
| No. | (eV) | (eV) | (eV) | (eV) | (eV) |
| 1 | −4.647 | −1.681 | 2.965 | 2.489 | 2.333 |
| 2 | −4.660 | −1.694 | 2.966 | 2.489 | 2.334 |
| 3 | −4.584 | −1.642 | 2.942 | 2.486 | 2.336 |
| 4 | −4.584 | −1.642 | 2.942 | 2.486 | 2.336 |
| 5 | −4.647 | −1.681 | 2.965 | 2.489 | 2.333 |
| 6 | −4.647 | −1.681 | 2.965 | 2.489 | 2.333 |
| 7 | −4.598 | −1.624 | 2.974 | 2.488 | 2.344 |
| 8 | −4.621 | −1.695 | 2.926 | 2.486 | 2.311 |

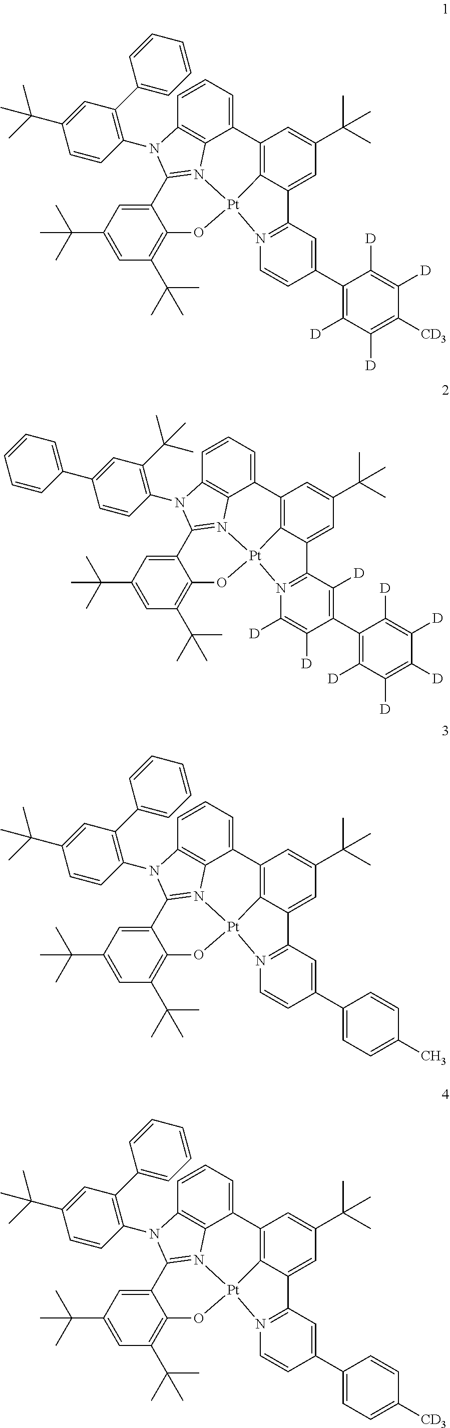

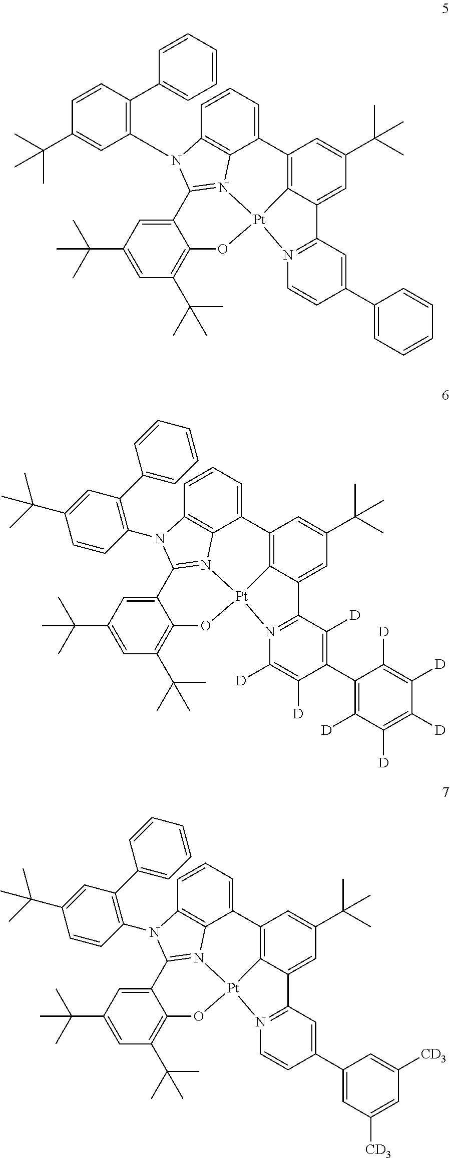

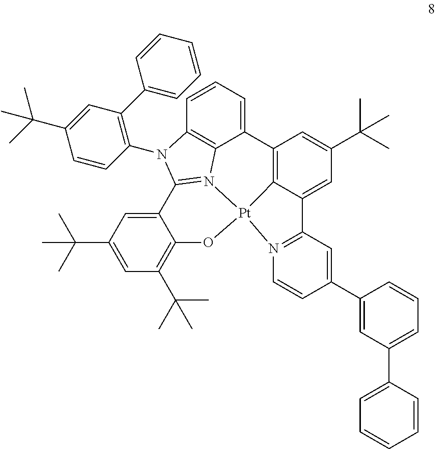

From Table 1, it is confirmed that the organometallic compound represented by Formula 1 has such electric characteristics that are suitable for use in an electronic device, for example, for use as a dopant for an organic light-emitting device.

Synthesis methods of the organometallic compound represented by Formula 1 may be understood by one of ordinary skill in the art by referring to Synthesis Examples provided below.

The organometallic compound represented by Formula 1 is suitable for use in an organic layer of an organic light-emitting device, for example, for use as a dopant in an emission layer of the organic layer. Thus, another aspect provides an organic light-emitting device that includes: a first electrode; a second electrode; and an organic layer that is disposed between the first electrode and the second electrode, wherein the organic layer includes an emission layer and an organometallic compound represented by Formula 1.

The organic light-emitting device may have, due to the inclusion of an organic layer including the organometallic compound represented by Formula 1, a low driving voltage, high quantum efficiency, a low roll-off ratio, and a long lifespan.

The organometallic compound of Formula 1 may be used between a pair of electrodes of an organic light-emitting device. For example, the organometallic compound represented by Formula 1 may be included in the emission layer. In this embodiment, the organometallic compound may act as a dopant, and the emission layer may further include a host (that is, an amount of the organometallic compound represented by Formula 1 is smaller than an amount of the host).

In an embodiment, the emission layer may include a host and a dopant, and the dopant may include the organometallic compound represented by Formula 1. The organometallic compound represented by Formula 1 may be a red phosphorescent dopant.

The expression “(an organic layer) includes an organometallic compound” as used herein may include an embodiment in which “(an organic layer) includes identical organometallic compounds represented by Formula 1” and an embodiment in which “(an organic layer) includes two or more different organometallic compounds represented by Formula 1.”

For example, the organic layer may include, as the organometallic compound, only Compound 1. In this embodiment, Compound 1 may be included in an emission layer of the organic light-emitting device. In an embodiment, the organic layer may include, as the organometallic compound, Compound 1 and Compound 2. In this embodiment, Compound 1 and Compound 2 may be included in an identical layer (for example, Compound 1 and Compound 2 may both be included in an emission layer).

The first electrode may be an anode, which is a hole injection electrode, and the second electrode may be a cathode, which is an electron injection electrode; or the first electrode may be a cathode, which is an electron injection electrode, and the second electrode may be an anode, which is a hole injection electrode.

In an embodiment, in the organic light-emitting device, the first electrode may be an anode, and the second electrode may be a cathode, and the organic layer may further include a hole transport region disposed between the first electrode and the emission layer and an electron transport region disposed between the emission layer and the second electrode, wherein the hole transport region may include a hole injection layer, a hole transport layer, an electron blocking layer, or any combination thereof, and the electron transport region may include a hole blocking layer, an electron transport layer, an electron injection layer, or any combination thereof.

The term “organic layer” as used herein refers to a single layer and/or a plurality of layers disposed between the first electrode and the second electrode of the organic light-emitting device. The “organic layer” may include, in addition to an organic compound, an organometallic complex including metal.

The FIGURE is a schematic view of an organic light-emitting device 10 according to an embodiment. Hereinafter, the structure of an organic light-emitting device according to an embodiment and a method of manufacturing an organic light-emitting device according to an embodiment will be described in connection with the FIGURE. The organic light-emitting device 10 includes a first electrode 11, an organic layer 15, and a second electrode 19, which are sequentially stacked.

A substrate may be additionally disposed under the first electrode 11 or above the second electrode 19. For use as the substrate, any substrate that is used in general organic light-emitting devices may be used, and the substrate may be a glass substrate or a transparent plastic substrate, each having excellent mechanical strength, thermal stability, transparency, surface smoothness, ease of handling, and water resistance.

The first electrode 11 may be formed by depositing or sputtering a material for forming the first electrode 11 on the substrate. The first electrode 11 may be an anode. The material for forming the first electrode 11 may be a material(s) with a high work function to facilitate hole injection. The first electrode 11 may be a reflective electrode, a semi-transmissive electrode, or a transmissive electrode. The material for forming the first electrode may be, for example, indium tin oxide (ITO), indium zinc oxide (IZO), tin oxide (SnO₂), and zinc oxide (ZnO). In an embodiment, magnesium (Mg), aluminum (Al), aluminum-lithium (Al-L₁), calcium (Ca), magnesium-indium (Mg—In), or magnesium-silver (Mg—Ag) may be used as the material for forming the first electrode.

The first electrode 11 may have a single-layered structure or a multi-layered structure including two or more layers. For example, the first electrode 11 may have a three-layered structure of ITO/Ag/ITO, but the structure of the first electrode 110 is not limited thereto.

The organic layer 15 is disposed on the first electrode 11.

The organic layer 15 may include a hole transport region, an emission layer, and an electron transport region.

The hole transport region may be disposed between the first electrode 11 and the emission layer.

The hole transport region may include a hole injection layer, a hole transport layer, an electron blocking layer, a buffer layer, or any combination thereof.

The hole transport region may include only either a hole injection layer or a hole transport layer. In an embodiment, the hole transport region may have a hole injection layer/hole transport layer structure or a hole injection layer/hole transport layer/electron blocking layer structure, which are sequentially stacked in this stated order from the first electrode 11.

A hole injection layer may be formed on the first electrode 11 by using a suitable method for example, vacuum deposition, spin coating, casting, Langmuir-Blodgett (LB) deposition, or any combination thereof.

When a hole injection layer is formed by vacuum deposition, the deposition conditions may vary according to a compound that is used to form the hole injection layer, and the structure and thermal characteristics of the hole injection layer. For example, the deposition conditions may include a deposition temperature of about 100° C. to about 500° C., a vacuum pressure of about 10⁻⁸ torr to about 10⁻³ torr, and a deposition rate of about 0.01 Angstroms per second (Å/sec) to about 100 Å/sec. However, the deposition conditions are not limited thereto.

When the hole injection layer is formed using spin coating, coating conditions may vary according to the material used to form the hole injection layer, and the structure and thermal properties of the hole injection layer. For example, a coating speed may be from about 2,000 revolutions per minute (rpm) to about 5,000 rpm, and a temperature at which a heat treatment is performed to remove a solvent after coating may be from about 80° C. to about 200° C. However, the coating conditions are not limited thereto.

Conditions for forming a hole transport layer and an electron blocking layer may be understood by referring to conditions for forming the hole injection layer.

The hole transport region may include m-MTDATA, TDATA, 2-TNATA, NPB, β-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzene sulfonic acid (PANI/DBSA), poly(3,4-ethylenedioxythiophene)/poly(4-styrene sulfonate) (PEDOT/PSS), polyaniline/camphor sulfonic acid (PANI/CSA), polyaniline/poly(4-styrene sulfonate) (PANI/PSS), a compound represented by Formula 201, a compound represented by Formula 202, or any combination thereof:

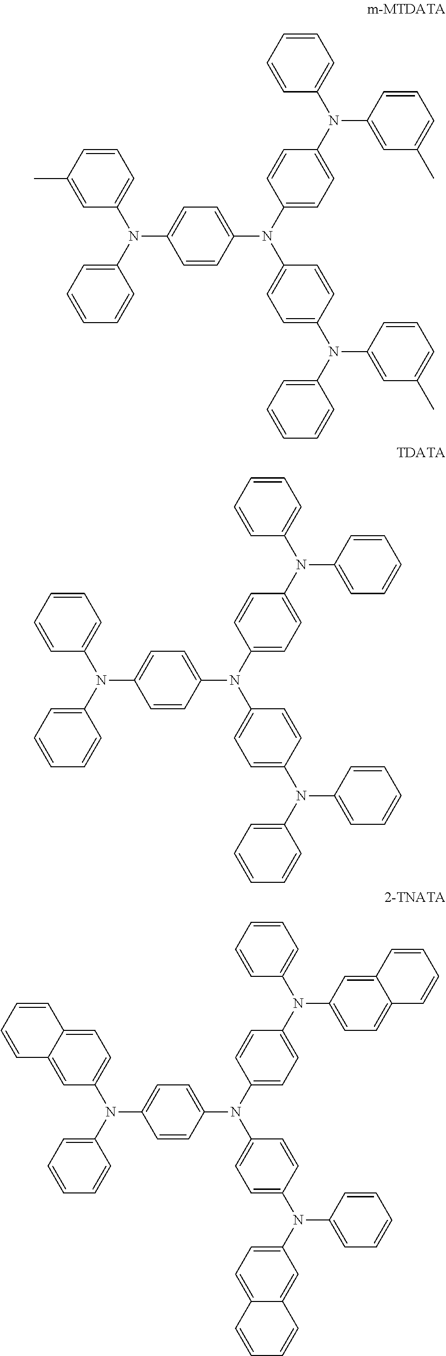

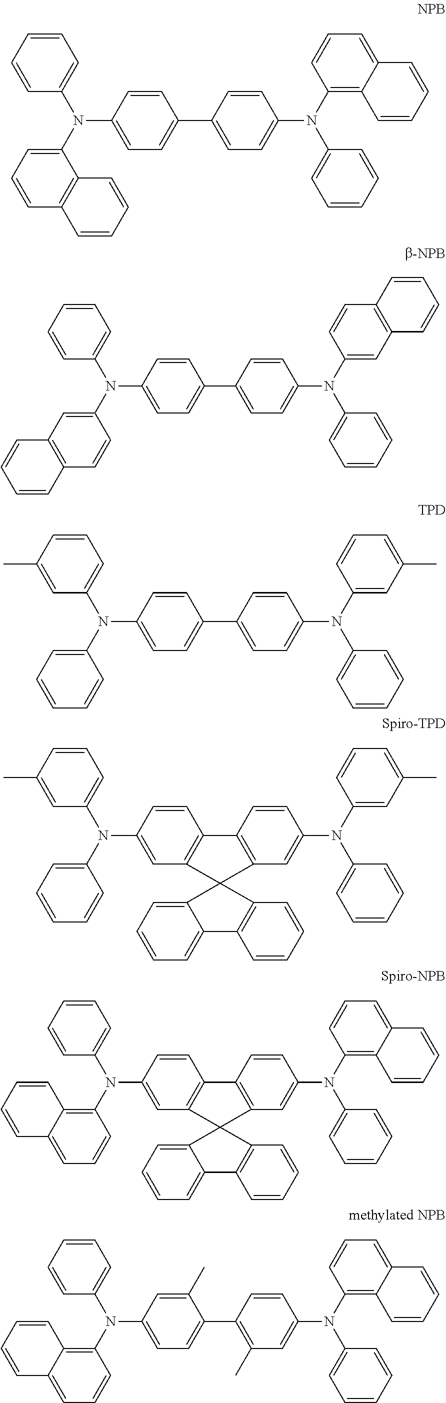

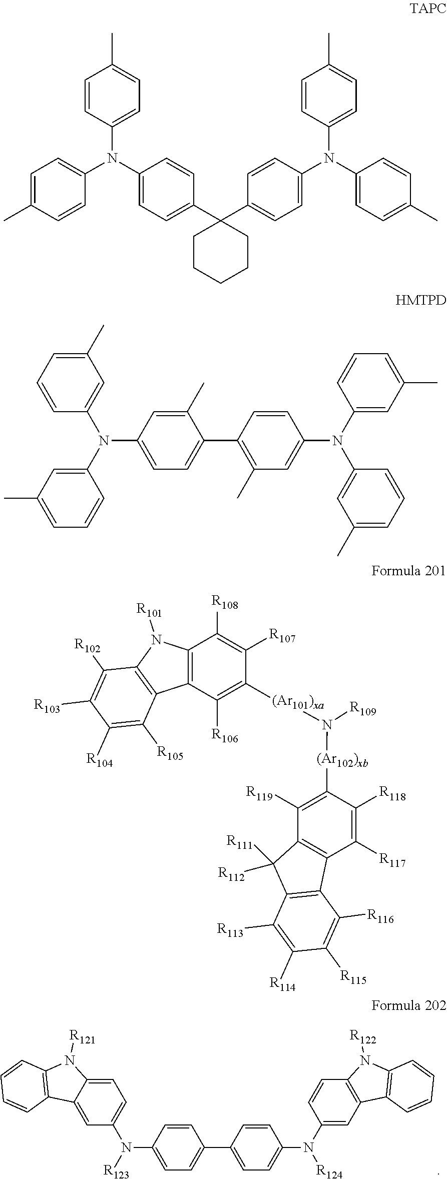

In Formula 201, Ar₁₀₁ and Ar₁₀₂ may each independently be a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, or a pentacenylene group, each unsubstituted or substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₂-C₁₀ heterocycloalkyl group, a C₂-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, a monovalent non-aromatic condensed heteropolycyclic group, or any combination thereof.

In Formula 201, xa and xb may each independently be an integer from 0 to 5, or may be 0, 1, or 2. For example, xa is 1 and xb is 0, but xa and xb are not limited thereto.

In Formulae 201 and 202, R₁₀₁ to R₁₀₈, R₁₁₁ to R₁₁₉, and R₁₂₁ to R₁₂₄ may each independently be:

a hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, a pentyl group, a hexyl group, and so on), or a C₁-C₁₀ alkoxy group (for example, a methoxy group, an ethoxy group, a propoxy group, a butoxy group, a pentoxy group, and so on);

a C₁-C₁₀ alkyl group or a C₁-C₁₀ alkoxy group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, or any combination thereof; or

a phenyl group, a naphthyl group, an anthracenyl group, a fluorenyl group, or a pyrenyl group, each unsubstituted or substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a C₁-C₁₀ alkoxy group or any combination thereof,

but embodiments of the present disclosure are not limited thereto.

In Formula 201, R₁₀₉ may be a phenyl group, a naphthyl group, an anthracenyl group, or a pyridinyl group, each unsubstituted or substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, a pyridinyl group, or any combination thereof.

In an embodiment, the compound represented by Formula 201 may be represented by Formula 201A, but embodiments of the present disclosure are not limited thereto:

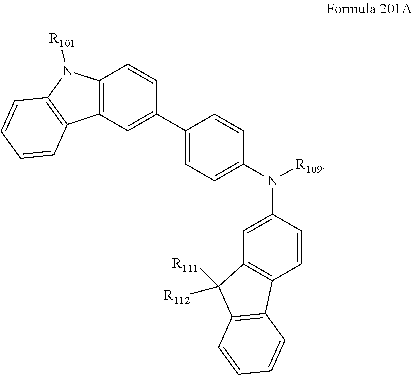

In Formula 201A, R₁₀₁, R₁₁₁, R₁₁₂, and R₁₀₉ are each independently the same as described above.

For example, the compound represented by Formula 201 and the compound represented by Formula 202 may each independently include Compounds HT1 to HT20, but are not limited thereto:

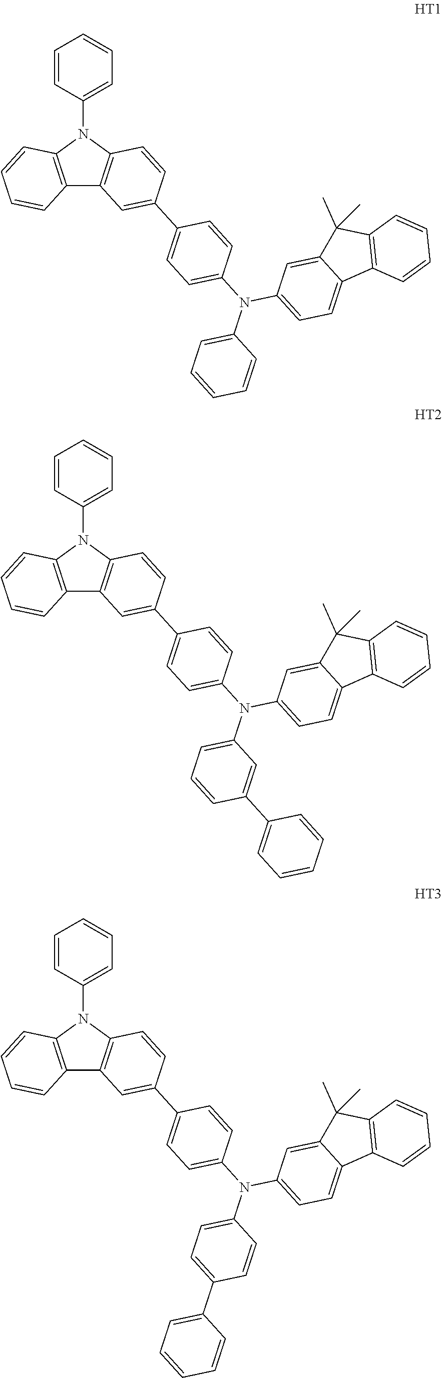 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

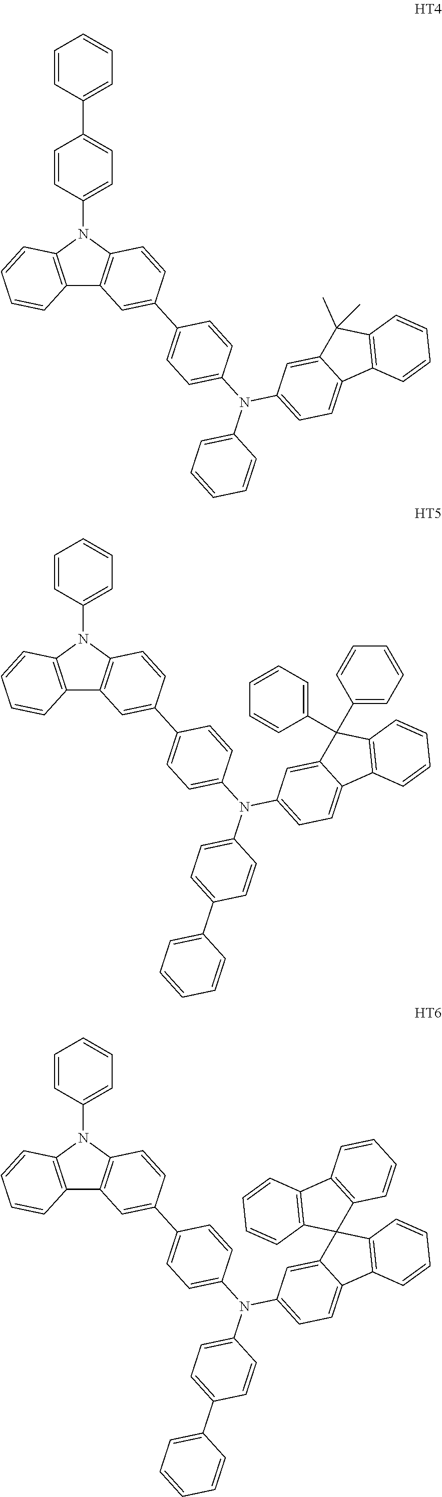 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

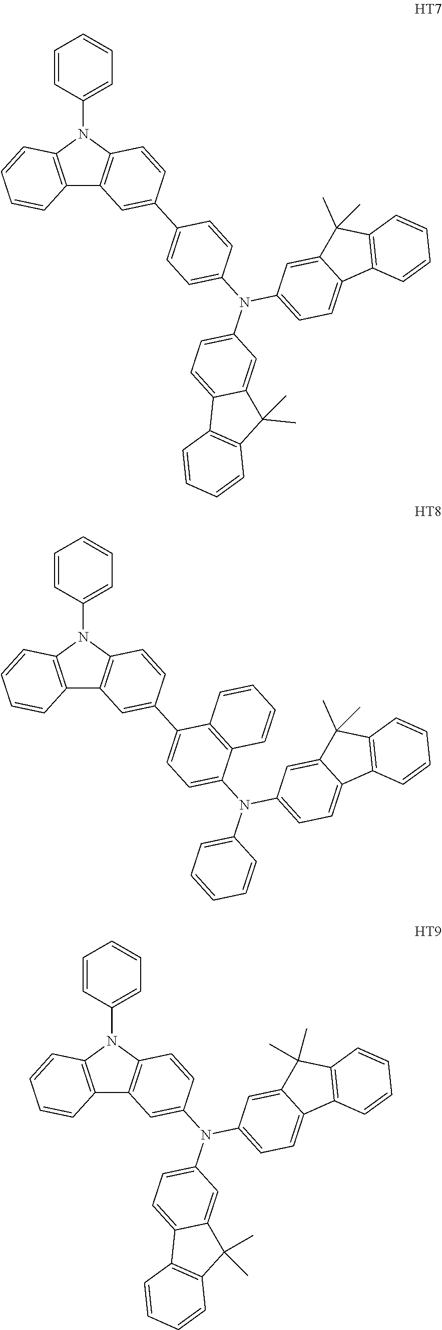 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccccc3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21]

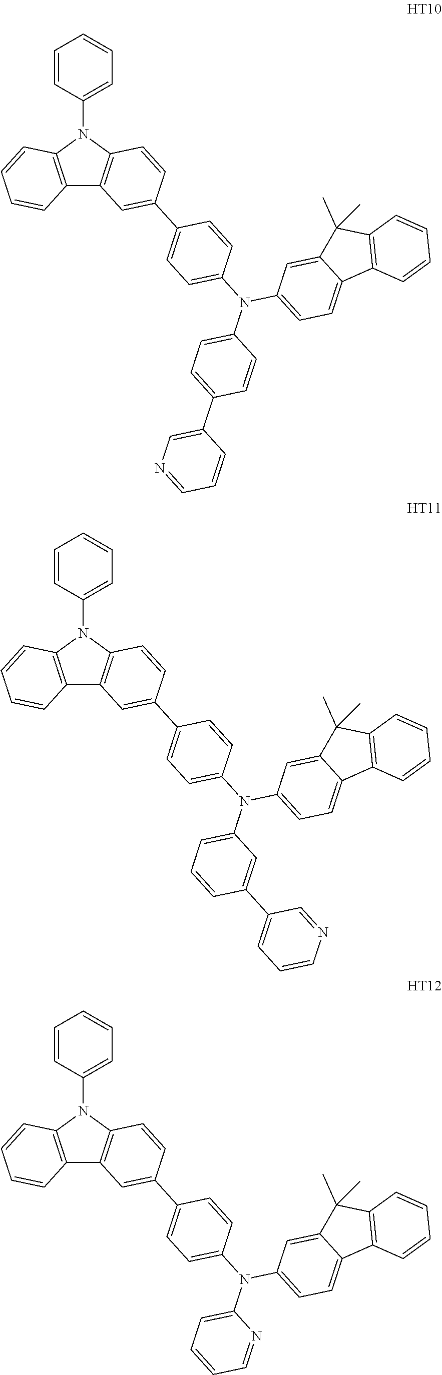 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21]

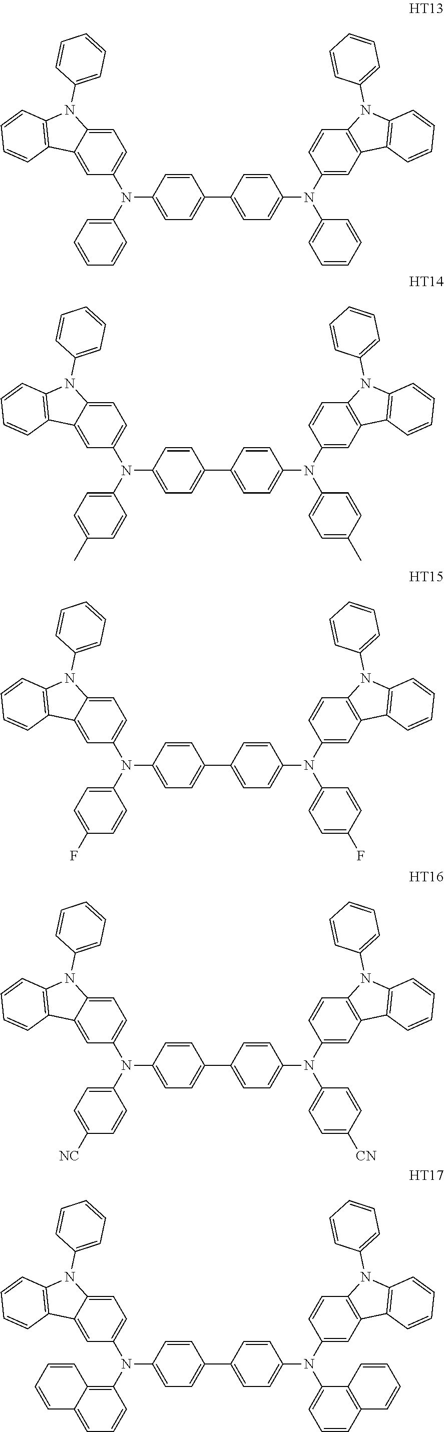

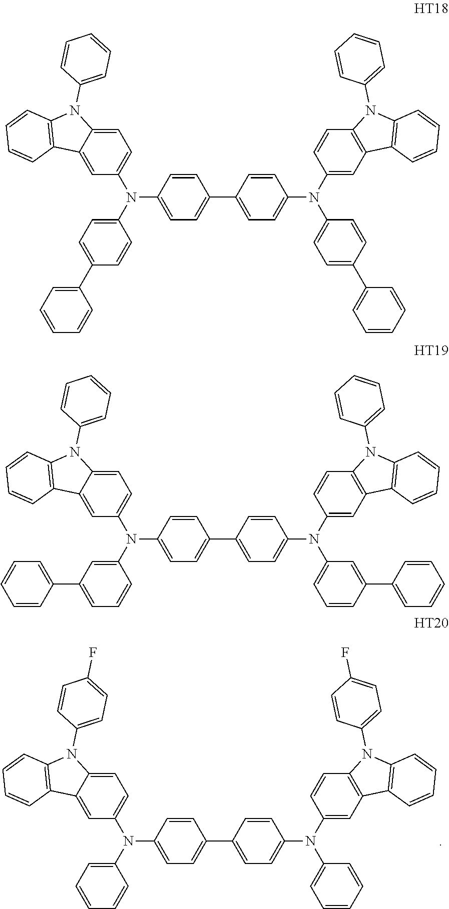 [SMILES: c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be in a range of about 100 Angstroms (Å) to about 10,000 Å, for example, about 100 Å to about 1,000 Å. When the hole transport region includes a hole injection layer, a hole transport layer, or any combination thereof, the thickness of the hole injection layer may be in a range of about 100 Å to about 10,000 Å, and for example, about 100 Å to about 1,000 Å, and the thickness of the hole transport layer may be in a range of about 50 Å to about 2,000 Å, and for example, about 100 Å to about 1,500 Å. While not wishing to be bound by theory, it is understood that when the thicknesses of the hole transport region, the hole injection layer, and the hole transport layer are within these ranges, satisfactory hole transporting characteristics may be obtained without a substantial increase in driving voltage.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be a quinone derivative, a metal oxide, a cyano group-containing compound, or any combination thereof, but embodiments of the present disclosure are not limited thereto. Non-limiting examples of the p-dopant are a quinone derivative, such as tetracyanoquinodimethane (TCNQ) or 2,3,5,6-tetrafluoro-tetracyano-1,4-benzoquinodimethane (F4-TCNQ); a metal oxide, such as a tungsten oxide or a molybdenum oxide; and a cyano group-containing compound, such as Compound HT-D₁, but are not limited thereto.

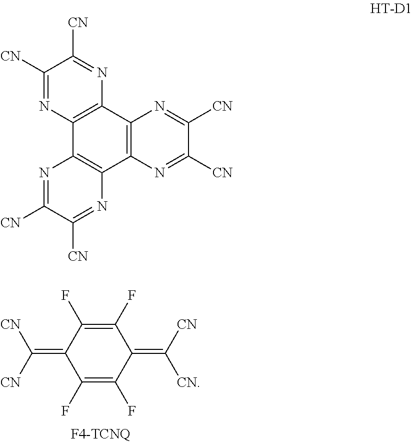

The hole transport region may include a buffer layer.

Also, the buffer layer may compensate for an optical resonance distance according to a wavelength of light emitted from the emission layer, and thus, efficiency of a formed organic light-emitting device may be improved.

Meanwhile, when the hole transport region includes an electron blocking layer, a material for the electron blocking layer may be a material(s) for the hole transport region described above and materials for a host to be explained later. However, the material for the electron blocking layer is not limited thereto. For example, when the hole transport region includes an electron blocking layer, a material for the electron blocking layer may be mCP, which will be explained later.

Then, an emission layer may be formed on the hole transport region by vacuum deposition, spin coating, casting, LB deposition, or the like. When the emission layer is formed by vacuum deposition or spin coating, the deposition or coating conditions may be similar to those applied in forming the hole injection layer although the deposition or coating conditions may vary according to a compound that is used to form the emission layer.

The emission layer may include a host and a dopant, and the dopant may include the organometallic compound represented by Formula 1.

The host may include TPBi, TBADN, ADN (also referred to as “DNA”), CBP, CDBP, TCP, mCP, Compound H50, Compound H51, or any combination thereof:

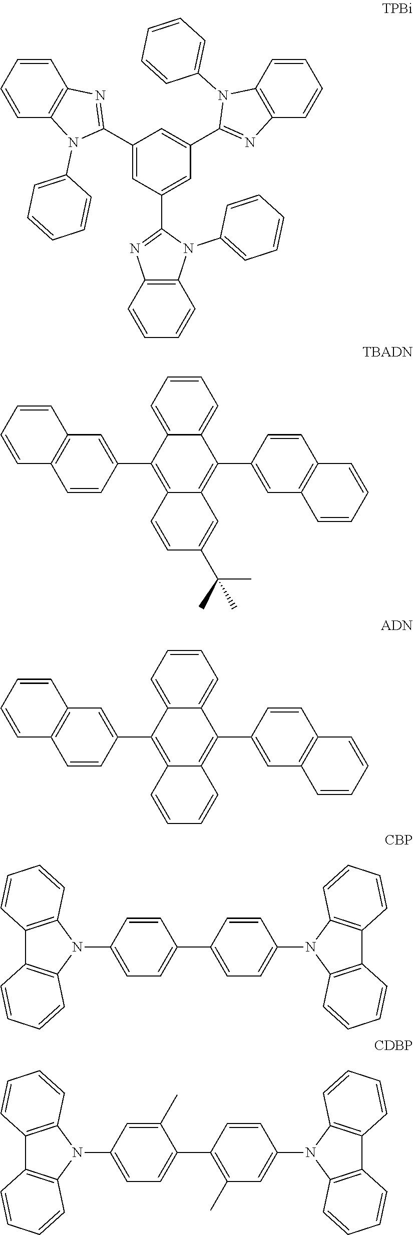

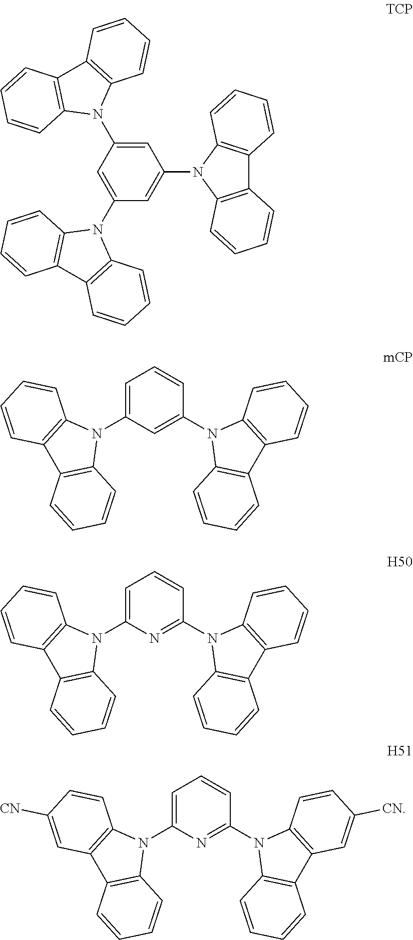

When the organic light-emitting device is a full-color organic light-emitting device, the emission layer may be patterned into a red emission layer, a green emission layer, and/or a blue emission layer. In an embodiment, due to a stacked structure including a red emission layer, a green emission layer, and/or a blue emission layer, the emission layer may emit white light.

When the emission layer includes a host and a dopant, an amount of the dopant may be in a range of about 0.01 parts by weight to about 15 parts by weight based on 100 parts by weight of the host, but embodiments of the present disclosure are not limited thereto.

The dopant may include the organometallic compound represented by Formula 1. For example, the dopant may be a red phosphorescent dopant.

A thickness of the emission layer may be in a range of about 100 Å to about 1,000 Å, for example, about 200 Å to about 600 Å. While not wishing to be bound by theory, it is understood that when the thickness of the emission layer is within this range, excellent light-emission characteristics may be obtained without a substantial increase in driving voltage.

Then, an electron transport region may be disposed on the emission layer.

The electron transport region may include a hole blocking layer, an electron transport layer, an electron injection layer, or any combination thereof.

For example, the electron transport region may have a hole blocking layer/electron transport layer/electron injection layer structure or an electron transport layer/electron injection layer structure, but the structure of the electron transport region is not limited thereto. The electron transport layer may have a single-layered structure or a multi-layered structure including two or more different materials.

Conditions for forming the hole blocking layer, the electron transport layer, and the electron injection layer which constitute the electron transport region may be understood by referring to the conditions for forming the hole injection layer.

When the electron transport region includes a hole blocking layer, the hole blocking layer may include, for example, BCP, Bphen, BAIq, or any combination thereof, but embodiments of the present disclosure are not limited thereto:

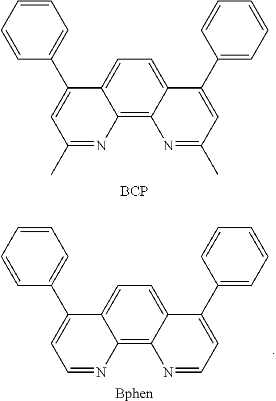

A thickness of the hole blocking layer may be in a range of about 20 Å to about 1,000 Å, for example, about 30 Å to about 300 Å. While not wishing to be bound by theory, it is understood that when the thickness of the hole blocking layer is within these ranges, the hole blocking layer may have improved hole blocking ability without a substantial increase in driving voltage.

The electron transport layer may include BCP, Bphen, Alq₃, BAIq, TAZ, NTAZ, or any combination thereof:

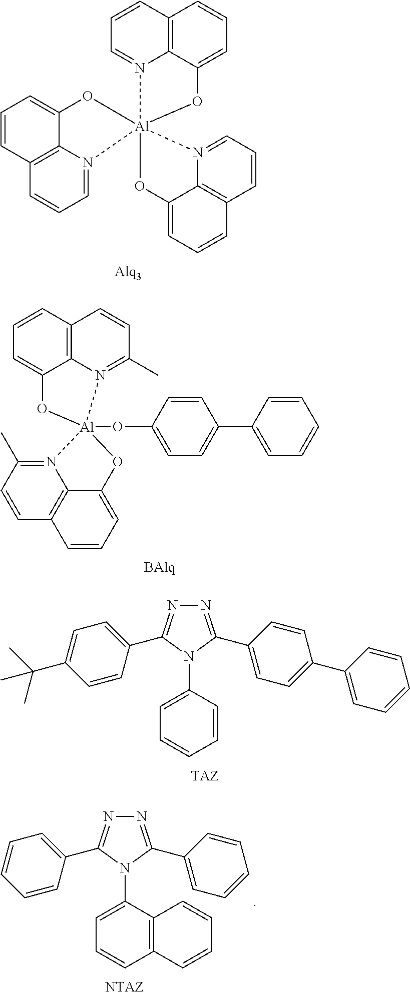

In an embodiment, the electron transport layer may include at least one of ET1 to ET25, but are not limited thereto:

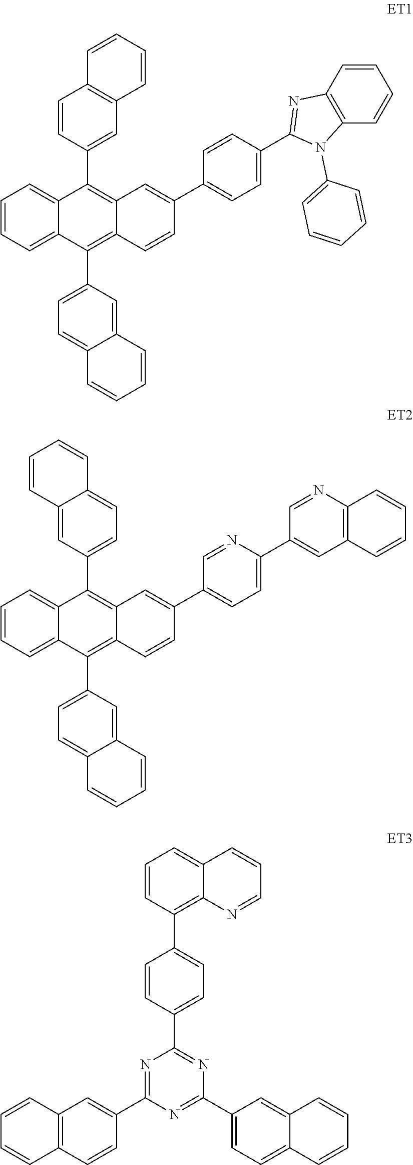 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc2cc(-c3nc(-c4ccc(-c5cccc6cccnc56)cc4)nc(-c4ccc5ccccc5c4)n3)ccc2c1]

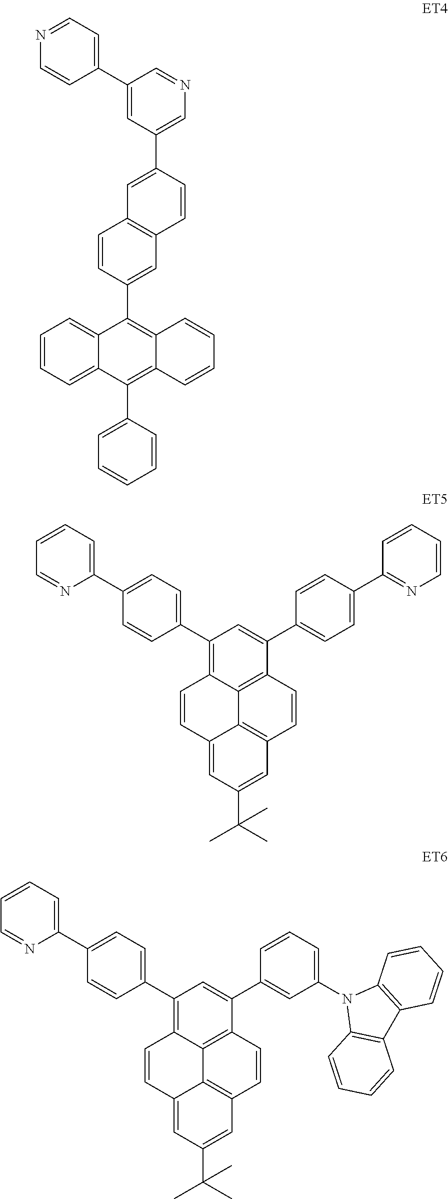 [SMILES: c1ccc(-c2c3ccccc3c(-c3ccc4cc(-c5cncc(-c6ccncc6)c5)ccc4c3)c3ccccc23)cc1 ; CC(C)(C)c1cc2ccc3c(-c4ccc(-c5ccccn5)cc4)cc(-c4ccc(-c5ccccn5)cc4)c4ccc(c1)c2c34 ; CC(C)(C)c1cc2ccc3c(-c4ccc(-c5ccccn5)cc4)cc(-c4cccc(-n5c6ccccc6c6ccccc65)c4)c4ccc(c1)c2c34]

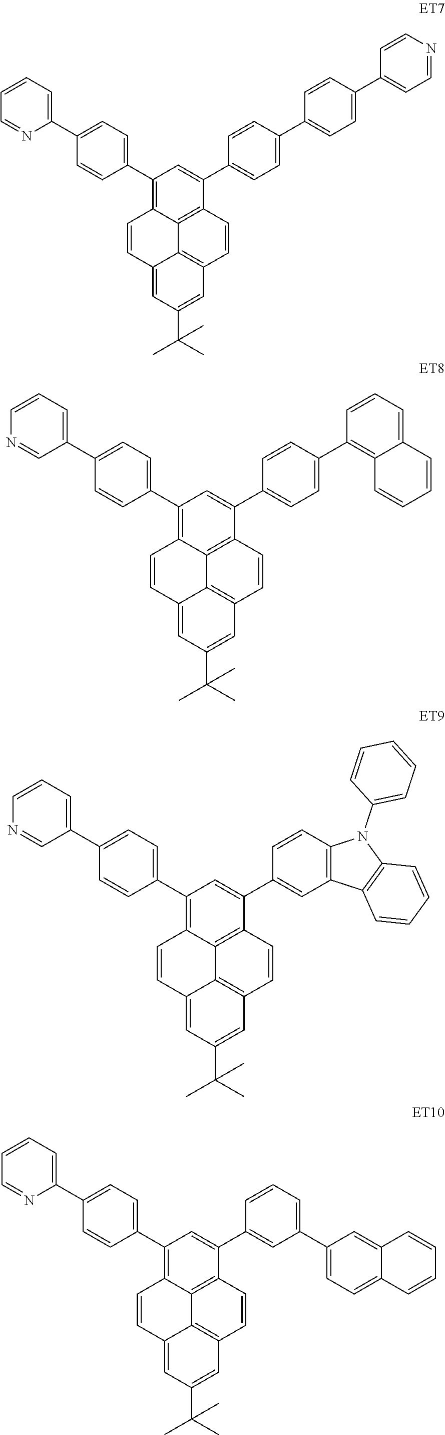 [SMILES: CC(C)(C)c1cc2ccc3c(-c4ccc(-c5ccc(-c6ccncc6)cc5)cc4)cc(-c4ccc(-c5ccccn5)cc4)c4ccc(c1)c2c34 ; CC(C)(C)c1cc2ccc3c(-c4ccc(-c5cccnc5)cc4)cc(-c4ccc(-c5cccc6ccccc56)cc4)c4ccc(c1)c2c34 ; CC(C)(C)c1cc2ccc3c(-c4ccc(-c5cccnc5)cc4)cc(-c4ccc5c(c4)c4ccccc4n5-c4ccccc4)c4ccc(c1)c2c34 ; CC(C)(C)c1cc2ccc3c(-c4ccc(-c5ccccn5)cc4)cc(-c4cccc(-c5ccc6ccccc6c5)c4)c4ccc(c1)c2c34]

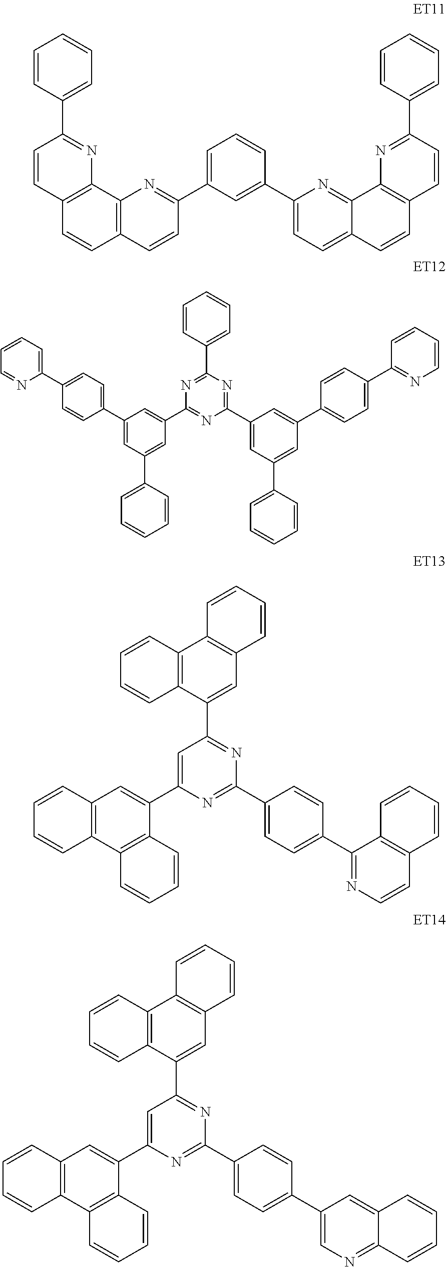 [SMILES: c1ccc(-c2ccc3ccc4ccc(-c5cccc(-c6ccc7ccc8ccc(-c9ccccc9)nc8c7n6)c5)nc4c3n2)cc1 ; c1ccc(-c2cc(-c3ccc(-c4ccccn4)cc3)cc(-c3nc(-c4ccccc4)nc(-c4cc(-c5ccccc5)cc(-c5ccc(-c6ccccn6)cc5)c4)n3)c2)cc1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)cc(-c5cc6ccccc6c6ccccc56)n4)cc3)cc2c1]

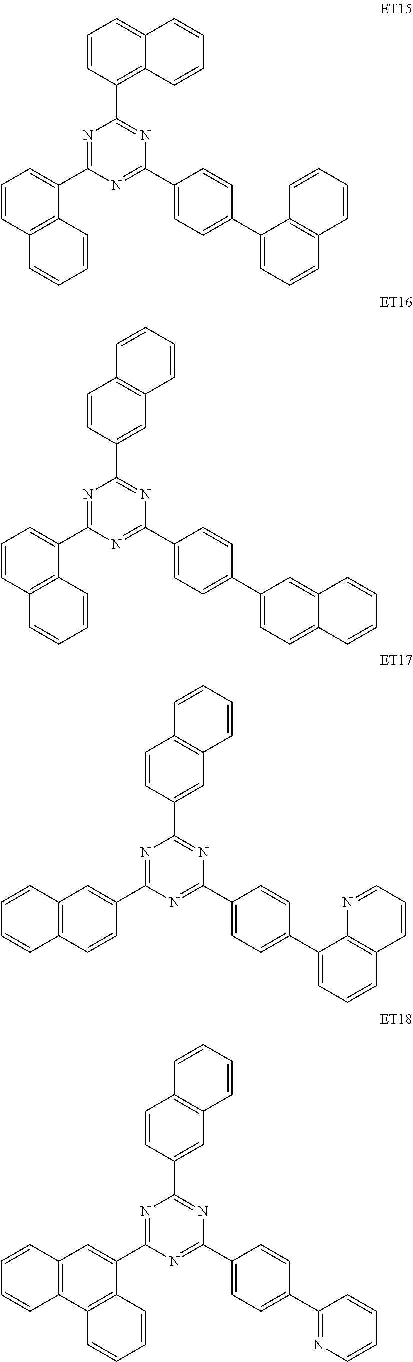 [SMILES: c1ccc2c(-c3ccc(-c4nc(-c5cccc6ccccc56)nc(-c5cccc6ccccc56)n4)cc3)cccc2c1 ; c1ccc2cc(-c3ccc(-c4nc(-c5ccc6ccccc6c5)nc(-c5cccc6ccccc56)n4)cc3)ccc2c1 ; c1ccc2cc(-c3nc(-c4ccc(-c5cccc6cccnc56)cc4)nc(-c4ccc5ccccc5c4)n3)ccc2c1 ; c1ccc(-c2ccc(-c3nc(-c4ccc5ccccc5c4)nc(-c4cc5ccccc5c5ccccc45)n3)cc2)nc1]

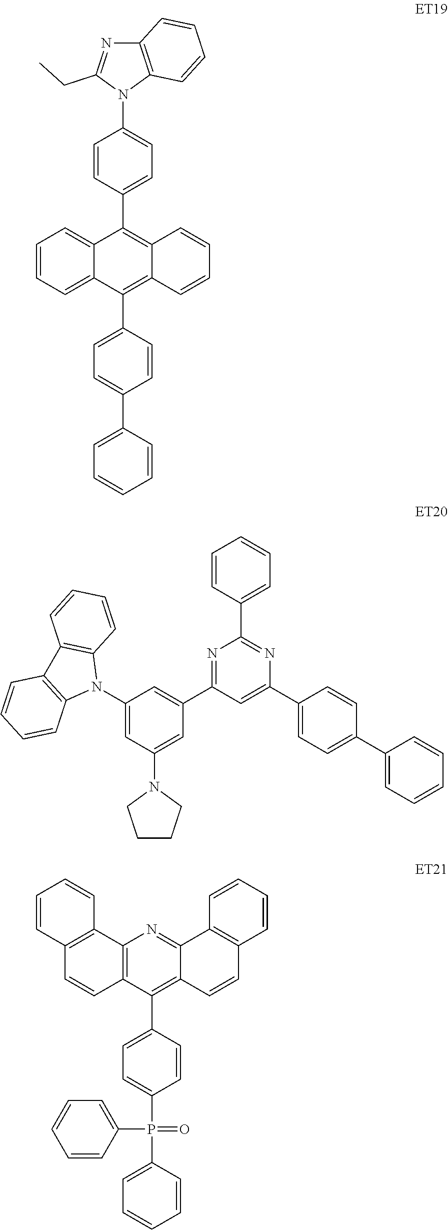 [SMILES: CCc1nc2ccccc2n1-c1ccc(-c2c3ccccc3c(-c3ccc(-c4ccccc4)cc3)c3ccccc23)cc1 ; c1ccc(-c2ccc(-c3cc(-c4cc(N5CCCC5)cc(-n5c6ccccc6c6ccccc65)c4)nc(-c4ccccc4)n3)cc2)cc1 ; O=P(c1ccccc1)(c1ccccc1)c1ccc(-c2c3ccc4ccccc4c3nc3c2ccc2ccccc23)cc1]

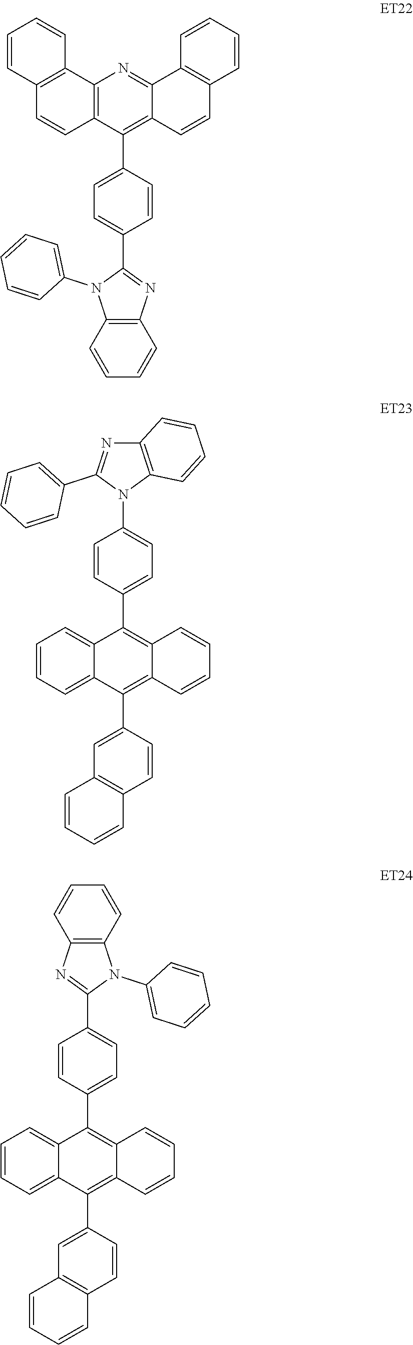

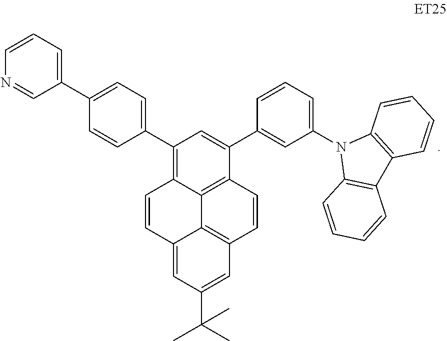 [SMILES: CC(C)(C)c1cc2ccc3c(-c4ccc(-c5cccnc5)cc4)cc(-c4cccc(-n5c6ccccc6c6ccccc65)c4)c4ccc(c1)c2c34]

A thickness of the electron transport layer may be in a range of about 100 Å to about 1,000 Å, for example, about 150 Å to about 500 Å. While not wishing to be bound by theory, it is understood that when the thickness of the electron transport layer is within the range described above, the electron transport layer may have satisfactory electron transport characteristics without a substantial increase in driving voltage.

Also, the electron transport layer may further include, in addition to the materials described above, a metal-containing material.

The metal-containing material may include a L₁ complex. The L₁ complex may include, for example, Compound ET-D₁ (lithium 8-hydroxyquinolate, LiQ) or ET-D₂:

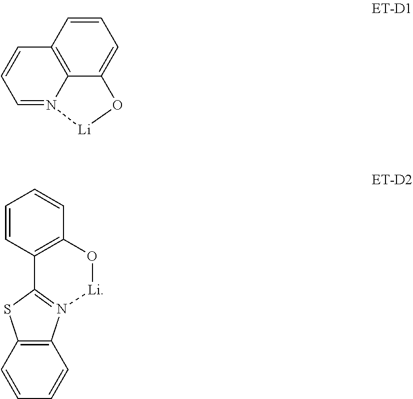

The electron transport region may include an electron injection layer that promotes flow of electrons from the second electrode 19 thereinto.

The electron injection layer may include LiF, NaCl, CsF, L₁₂O, BaO, or any combination thereof.

A thickness of the electron injection layer may be in a range of about 1 Å to about 100 Å, for example, about 3 Å to about 90 Å. While not wishing to be bound by theory, it is understood that when the thickness of the electron injection layer is within the range described above, the electron injection layer may have satisfactory electron injection characteristics without a substantial increase in driving voltage.

The second electrode 19 is disposed on the organic layer 15. The second electrode 19 may be a cathode. A material for forming the second electrode 19 may be metal, an alloy, an electrically conductive compound, or any combination thereof, which have a relatively low work function. For example, lithium (L₁), magnesium (Mg), aluminum (Al), aluminum-lithium (Al-L₁), calcium (Ca), magnesium-indium (Mg—In), or magnesium-silver (Mg—Ag) may be used as a material for forming the second electrode 19. In an embodiment, to manufacture a top-emission type light-emitting device, a transmissive electrode formed using ITO or IZO may be used as the second electrode 19.

Hereinbefore, the organic light-emitting device has been described with reference to the FIGURE, but embodiments of the present disclosure are not limited thereto.

Another aspect of the present disclosure provides a diagnostic composition including an organometallic compound represented by Formula 1.

The organometallic compound represented by Formula 1 provides high luminescent efficiency. Accordingly, a diagnostic composition including the organometallic compound may have high diagnostic efficiency.

The diagnostic composition may be used in various applications including a diagnosis kit, a diagnosis reagent, a biosensor, and a biomarker.

The term “C₁-C₆₀ alkyl group” as used herein refers to a linear or branched saturated aliphatic hydrocarbon monovalent group having 1 to 60 carbon atoms, and non-limiting examples thereof include a methyl group, an ethyl group, a propyl group, an iso-butyl group, a sec-butyl group, a tert-butyl group, a pentyl group, an iso-amyl group, and a hexyl group. The term “C₁-C₆₀ alkylene group” as used herein refers to a divalent group having the same structure as the C₁-C₆₀ alkyl group.

The term “C₁-C₆₀ alkoxy group” as used herein refers to a monovalent group represented by —OA₁₀₁(wherein A₁₀₁ is the C₁-C₆₀ alkyl group), and non-limiting examples thereof include a methoxy group, an ethoxy group, and an iso-propyloxy group.

The term “C₂-C₆₀ alkenyl group” as used herein refers to a hydrocarbon group formed by including a carbon-carbon double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group formed by including a carbon-carbon triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon monocyclic group having 3 to 10 carbon atoms, and non-limiting examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₂-C₁₀ heterocycloalkyl group” as used herein refers to a monovalent saturated monocyclic group having an N, O, P, Si, Se, S, or any combination thereof as a ring-forming atom and 1 to 10 carbon atoms, and non-limiting examples thereof include a tetrahydrofuranyl group, and a tetrahydrothiophenyl group. The term “C₂-C₁₀ heterocycloalkylene group” as used herein refers to a divalent group having the same structure as the C₂-C₁₀ heterocycloalkyl group.

The term “C₃-C₁₀ cycloalkenyl group” as used herein refers to a monovalent monocyclic group that has 3 to 10 carbon atoms and a carbon-carbon double bond in the ring thereof and that has no aromaticity, and non-limiting examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₂-C₁₀ heterocycloalkenyl group” as used herein refers to a monovalent monocyclic group that has an N, O, P, Si, Se, S, or any combination thereof as a ring-forming atom, 1 to 10 carbon atoms, and a carbon-carbon double bond in its ring. Examples of the C₂-C₁₀ heterocycloalkenyl group are a 2,3-dihydrofuranyl group, and a 2,3-dihydrothiophenyl group. The term “C₁-C₁₀ heterocycloalkenylene group” as used herein refers to a divalent group having the same structure as the C₂-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₇-C₆₀ alkylaryl group” as used herein refers to a C₁-C₆₀ aryl group substituted with a C₁-C₅₄ alkyl group.

The term “C₁-C₆₀ heteroaryl group” as used herein refers to a monovalent group having an aromatic system that has an N, O, P, Si, Se, S, or any combination thereof as a ring-forming atom, and 1 to 60 carbon atoms. The term “C₁-C₆₀ heteroarylene group” as used herein refers to a divalent group having an aromatic system that has an N, O, P, Se, S, or any combination thereof as a ring-forming atom, and 1 to 60 carbon atoms. Non-limiting examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include two or more rings, the rings may be fused to each other.

The term “C₂-C₆₀ alkylheteroaryl group” as used herein refers to a C₁-C₆₀ heteroaryl group substituted with a C₁-C₆₀ alkyl group.

The term “C₆-C₆₀ aryloxy group” as used herein indicates —OA₁₀₂ (wherein A₁₀₂ is the C₆-C₆₀ aryl group), a C₆-C₆₀ arylthio group as used herein indicates —SA₁₀₃ (wherein A₁₀₃ is the C₆-C₆₀ aryl group), and the term “C₇-C₆₀ arylalkyl group” as used herein indicates —A₁₀₄A₁₀₅ (wherein A₁₀₅ is the C₆-C₅₉ aryl group and A₁₀₄ is the C₁-C₅₄ alkylene group).

The term “C₁-C₆₀ heteroaryloxy group” as used herein refers to —OA₁₀₆ (wherein A₁₀₆ is the C₂-C₆₀ heteroaryl group), the term “C₁-C₆₀ heteroarylthio group” as used herein indicates —SA₁₀₇ (wherein A₁₀₇ is the C₁-C₆₀ heteroaryl group), and the term “C₂-C₆₀ heteroarylalkyl group” as used herein refers to —A₁₀₈A₁₀₉ (A₁₀₉ is a C₁-C₅₉ heteroaryl group, and A₁₀₈ is a C₁-C₅₉ alkylene group).

The term “monovalent non-aromatic condensed polycyclic group” as used herein refers to a monovalent group (for example, having 8 to 60 carbon atoms) having two or more rings condensed to each other, only carbon atoms as ring-forming atoms, and having no aromaticity in its entire molecular structure. Examples of the monovalent non-aromatic condensed polycyclic group include a fluorenyl group. The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group (for example, having 2 to 60 carbon atoms) having two or more rings condensed to each other, an N, O, P, Si, Se, S, or any combination thereof and carbon atoms, as ring-forming atoms, and no aromaticity in its entire molecular structure. Non-limiting examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group.

The term “C₅-C₃₀ carbocyclic group” as used herein refers to a saturated or unsaturated cyclic group having, as a ring-forming atom, 5 to 30 carbon atoms only. The C₅-C₃₀ carbocyclic group may be a monocyclic group or a polycyclic group.

The term “C₂-C₃₀ heterocyclic group” as used herein refers to a saturated or unsaturated cyclic group having, as a ring-forming atom, an N, O, Si, P, Se, S, or any combination thereof and 1 to 30 carbon atoms. The C₂-C₃₀ heterocyclic group may be a monocyclic group or a polycyclic group.

A substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₇-C₆₀ arylalkyl group, the substituted C₁-C₆₀ heteroaryl group, the substituted C₇-C₆₀ arylalkyl group, the substituted C₁-C₆₀ heteroaryl group, the substituted C₁-C₆₀ heteroaryloxy group, the substituted C₁-C₆₀ heteroarylthio group, the substituted C₂-C₆₀ heteroarylalkyl group, the substituted monovalent non-aromatic condensed polycyclic group, and the substituted monovalent non-aromatic condensed heteropolycyclic group may be:

a deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;

a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or any combination thereof;

a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, and a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or any combination thereof;

—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or

any combination thereof. For example, a CH₂CN group is a C₁ group substituted with a nitrile.

Q₁ to Q₉, Q₁₁ to Q₁₉, Q₂₁ to Q₂₉, and Q₃₁ to Q₃₉ in this disclosure may each independently be hydrogen; deuterium; —F; —Cl; —Br; —I; a hydroxyl group; a cyano group; a nitro group; an amidino group; a hydrazine group; a hydrazone group; a carboxylic acid group or a salt thereof; a sulfonic acid group or a salt thereof; a phosphoric acid group or a salt thereof; a C₁-C₆₀ alkyl group unsubstituted or substituted with deuterium, a C₁-C₆₀ alkyl group, a C₆-C₆₀ aryl group, or any combination thereof; a C₂-C₆₀ alkenyl group; a C₂-C₆₀ alkynyl group; a C₁-C₆₀ alkoxy group; a C₃-C₁₀ cycloalkyl group; a C₂-C₁₀ heterocycloalkyl group; a C₃-C₁₀ cycloalkenyl group; a C₂-C₁₀ heterocycloalkenyl group; a C₆-C₆₀ aryl group unsubstituted or substituted with deuterium, a C₁-C₆₀ alkyl group, a C₆-C₆₀ aryl group, or any combination thereof; a C₆-C₆₀ aryloxy group; a C₆-C₆₀ arylthio group; a C₇-C₆₀ arylalkyl group; a C₁-C₆₀ heteroaryl group; a C₁-C₆₀ heteroaryloxy group; a C₁-C₆₀ heteroarylthio group; a C₂-C₆₀ heteroarylalkyl group; a monovalent non-aromatic condensed polycyclic group; or a monovalent non-aromatic condensed heteropolycyclic group.

Hereinafter, a compound and an organic light-emitting device according to embodiments are described in detail with reference to Synthesis Example and Examples. However, the organic light-emitting device is not limited thereto. The expression “‘A’ is used instead of ‘B’” used in describing Synthesis Examples means that a molar equivalent of “A” was identical to a molar equivalent of “B”.

### EXAMPLES

### Synthesis Example 1 (Compound 6)

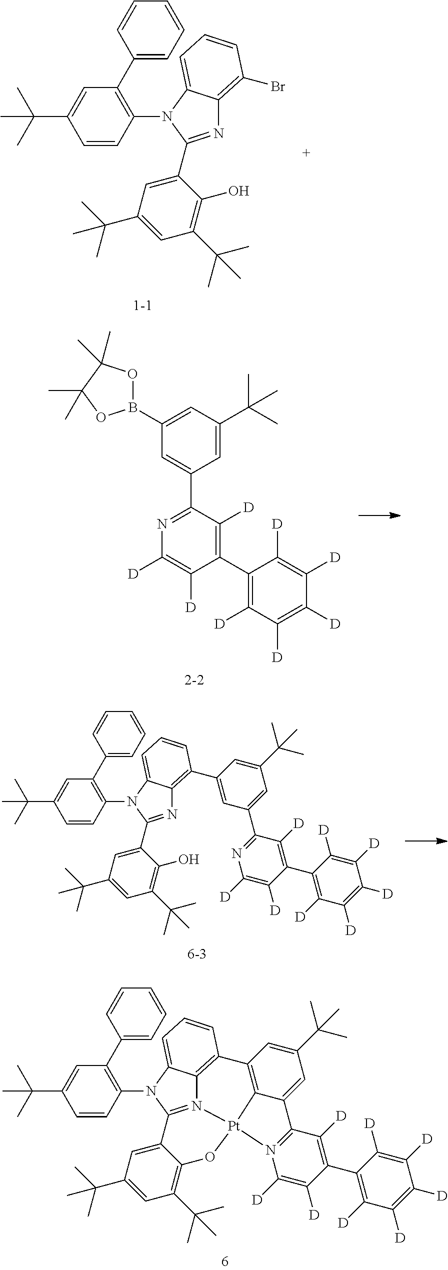

Synthesis of Intermediate 6-3

2.4 grams (g) (0.006 millimoles (mmol), 1.2 equivalents, equiv.) of Intermediate 2-2, 1.4 g (0.005 mmol, 1 equiv.) of Intermediate 1-1, 0.40 g (0.001 mmol, 0.07 equiv.) of tetrakis(triphenylphosphine)palladium(0), and 2.0 g (0.015 mmol, 3 equiv.) of potassium carbonate were dissolved in 20 milliliters (mL) of a solvent in which tetrahydrofuran (THF) and distilled water (H₂O) were mixed at a ratio of 3:1, and the reaction mixture was refluxed for 12 hours. The reaction mixture obtained therefrom was cooled to room temperature, and the precipitate was filtered to obtain a filtrate. The filtrate was washed by using ethyl acetate (EA)/H₂O and the crude product was purified by column chromatography (gradient elution of EA/hexane (Hex) from 20% to 35%) to obtain 2.2 g (yield of 68%) of Intermediate 6-3. The obtained compound was identified by Mass spectrum and HPLC analysis.

HRMS (MALDI) calcd for C₅₈H₅₃D₈N₃O m/z 823.5317, Found: 823.5319.

Synthesis of Compound 6

2.2 g (2.26 mmol) of Intermediate 6-3 and 1.1 g (2.26 mmol, 1.0 equiv.) of K₂PtCl₄ were dissolved in 40 mL of a solvent in which 30 mL of AcOH and 10 mL of H₂O were mixed, and the reaction mixture was refluxed for 16 hours. The reaction mixture obtained therefrom was cooled to room temperature, and the precipitate was filtered. The precipitate was dissolved again in MC and washed by using H₂O. The crude product was purified by column chromatography (methylene chloride (MC) 40%, EA 1%, Hex 59%) to obtain 1.6 g (yield of 60%) of Compound 6. The obtained compound was identified by Mass spectrum and HPLC analysis.

HRMS (MALDI) calcd for C₅₈H₅₁D₈N₃OPt: m/z 1016.4808, Found: 1016.4805.

### Synthesis Example 2 (Compound 1)

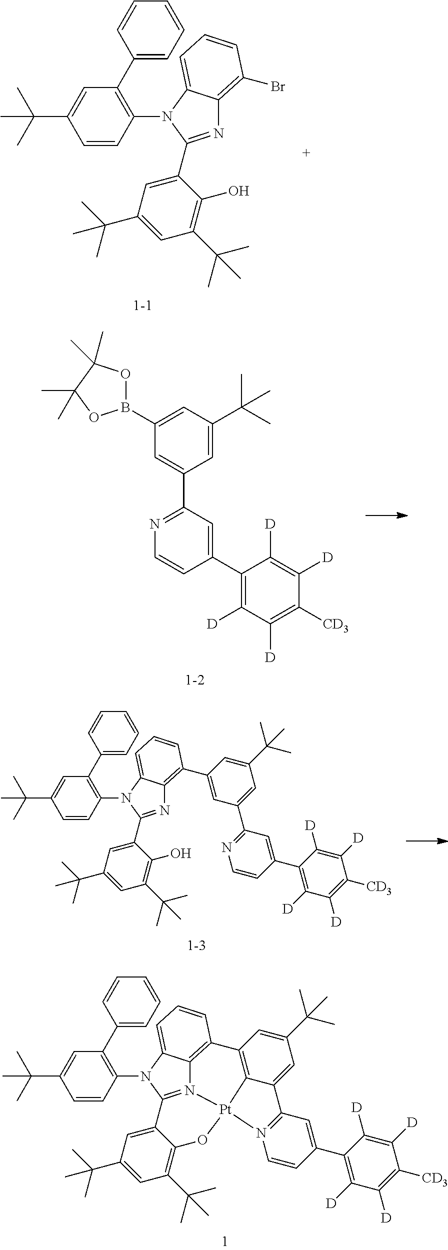

Synthesis of Intermediate 1-3

Intermediate 1-3 1.2 g (yield of 65%) was obtained in the same manner as in the synthesis of Intermediate 6-3 of Synthesis Example 1, except that Intermediate 1-2 was used instead of Intermediate 2-2. The obtained compound was identified by Mass spectrum and HPLC analysis.

HRMS (MALDI) calcd for C₅₉H₅₆D₇N₃O: m/z 836.5411, Found: 836.5414.

Synthesis of Compound 1

Compound 1 1.0 g (yield of 68%) was obtained in the same manner as in the synthesis of Compound 6 of Synthesis Example 1, except that Intermediate 1-3 was used instead of Intermediate 6-3. The obtained compound was identified by mass spectrum and HPLC analysis.

HRMS (MALDI) calcd for C₅₉H₅₄D₇N₃OPt: m/z 1029.4902, Found: 1029.4901.

### Synthesis Example 3 (Compound 2)

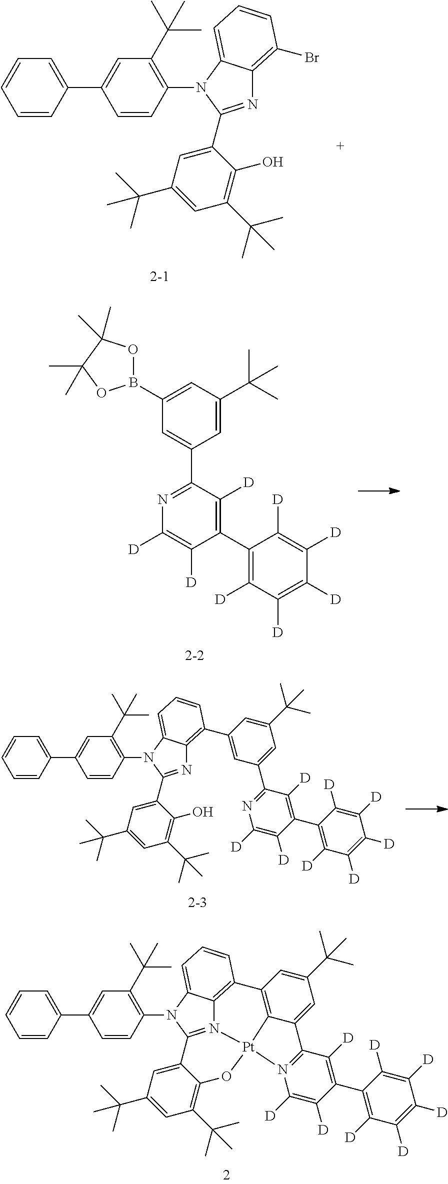

Synthesis of Intermediate 2-3

Intermediate 2-3 2.1 g (yield of 70%) was obtained in the same manner as in the synthesis of Intermediate 6-3 of Synthesis Example 1, except that Intermediate 2-1 was used instead of Intermediate 1-1. The obtained compound was identified by Mass spectrum and HPLC analysis.

HRMS (MALDI) calcd for C₅₈H₅₃D₈N₃O: m/z 823.5317, Found: 823.5319.

Synthesis of Compound 2

Compound 2 1.84 g (yield of 71%) was obtained in the same manner as in the synthesis of Compound 6 of Synthesis Example 1, except that Intermediate 2-3 was used instead of Intermediate 6-3. The obtained compound was identified by Mass spectrum and HPLC analysis.

HRMS (MALDI) calcd for C₅₈H₅₁D₈N₃OPt: m/z 1016.4808, Found: 1016.4809.

### Synthesis Example 4 (Compound 3)

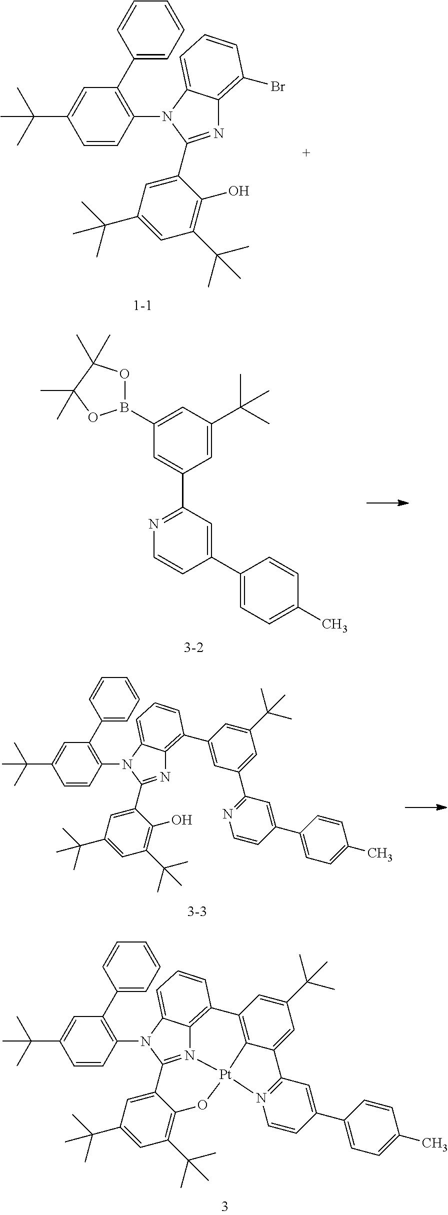

Synthesis of Intermediate 3-3

Intermediate 3-3 1.7 g (yield of 72%) was obtained in the same manner as in the synthesis of Intermediate 6-3 of Synthesis Example 1, except that Intermediate 3-2 was used instead of Intermediate 2-2. The obtained compound was identified by Mass and HPLC analysis.

HRMS (MALDI) calcd for C₅₉H₆₃N₃O: m/z 829.4971, Found: 829.4973.

Synthesis of Compound 3

Compound 3 1.53 g (yield of 73%) was obtained in the same manner as in the synthesis of Compound 6 of Synthesis Example 1, except that Intermediate 3-3 was used instead of Intermediate 6-3. The obtained compound was identified by Mass spectrum and HPLC analysis.

HRMS (MALDI) calcd for C₅₉H₆₁N₃OPt: m/z 1022.4462, Found: 1022.4461.

### Synthesis Example 5 (Compound 4)

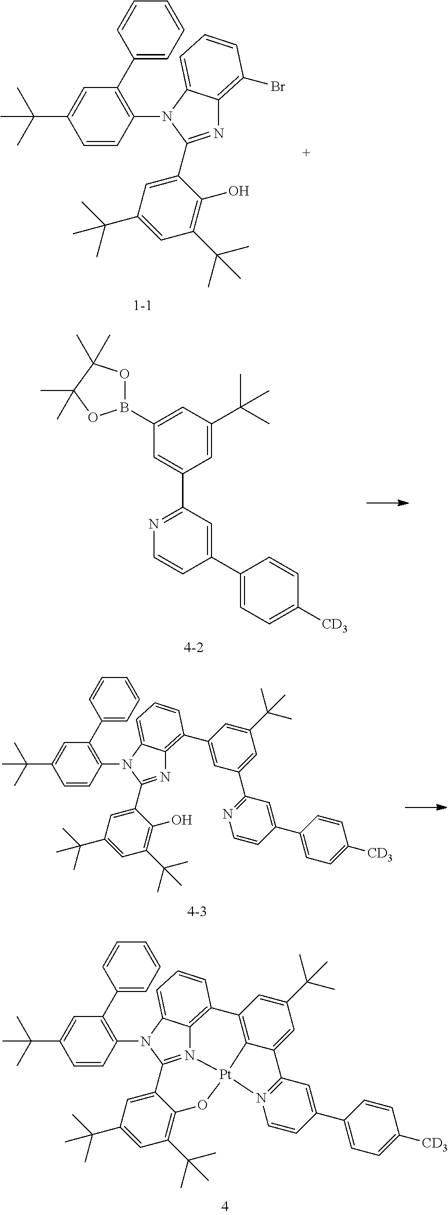

Synthesis of Intermediate 4-3

Intermediate 4-3 1.85 g (yield of 73%) was obtained in the same manner as in the synthesis of Intermediate 6-3 of Synthesis Example 1, except that Intermediate 4-2 was used instead of Intermediate 2-2. The obtained compound was identified by Mass spectrum and HPLC analysis.

HRMS (MALDI) calcd for C₅₉H₆₀D₃N₃O: m/z 832.5159, Found 832.52.

Synthesis of Compound 4

Compound 4 1.64 g (yield of 72%) was obtained in the same manner as in the synthesis of Compound 6 of Synthesis Example 1, except that Intermediate 4-3 was used instead of Intermediate 6-3. The obtained compound was identified by Mass spectrum and HPLC analysis.

HRMS (MALDI) calcd for C₅₉H₅₈D₃N₃OPt: m/z 1025.4651, Found: 1025.4652.

### Synthesis Example 6 (Compound 5)

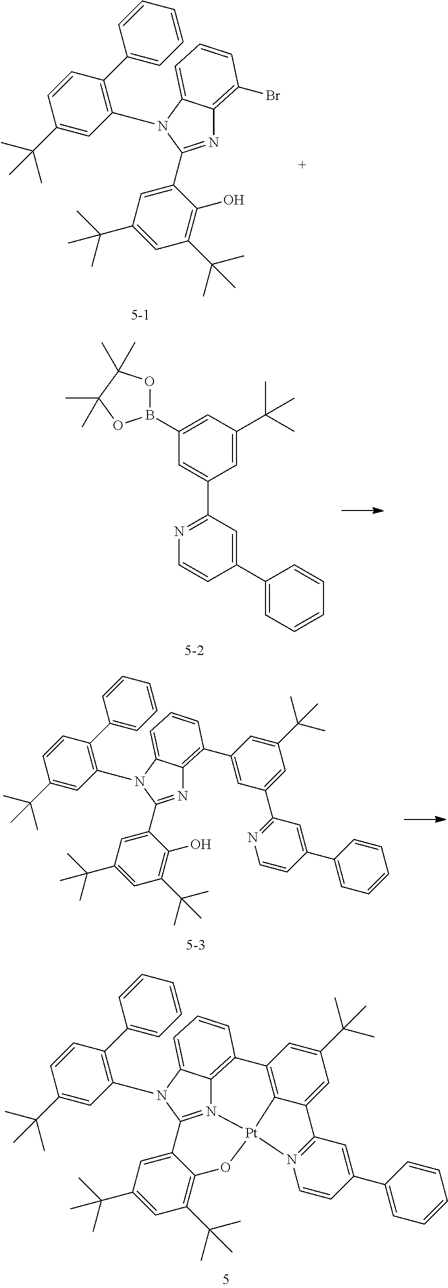

Synthesis of Intermediate 5-3

Intermediate 5-3 2 g (yield of 68%) was obtained in the same manner as in the synthesis of Intermediate 6-3 of Synthesis Example 1, except that Intermediate 5-1 and Intermediate 5-2 were used instead of Intermediate 1-1 and Intermediate 2-2. The obtained compound was identified by Mass and HPLC analysis.

HRMS (MALDI) calcd for C₅₈H₆₁N₃O: m/z 815.4815, Found: 815.4813.

Synthesis of Compound 5

Compound 5 1.73 g (yield of 70%) was obtained in the same manner as in the synthesis of Compound 6 of Synthesis Example 1, except that Intermediate 5-3 was used instead of Intermediate 6-3. The obtained compound was identified by Mass spectrum and HPLC analysis.

HRMS (MALDI) calcd for C₅₈H₅₉N₃OPt: m/z 1008.4306, Found: 1008.4308.

### Synthesis Example 7 (Compound 7)

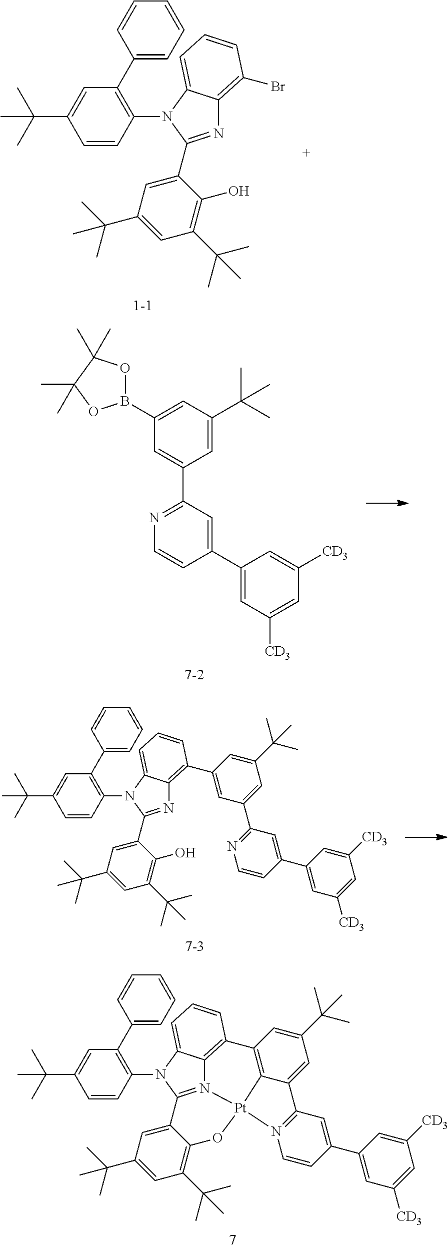

Synthesis of Intermediate 7-3

Intermediate 7-3 1.8 g (yield of 69%) was obtained in the same manner as in the synthesis of Intermediate 6-3 of Synthesis Example 1, except that Intermediate 7-2 was used instead of Intermediate 2-2. The obtained compound was identified by Mass spectrum and HPLC analysis.

HRMS (MALDI) calcd for C₆₀H₅₉D₆N₃O: m/z 849.5504, Found: 849.5502.

Synthesis of Compound 7

Compound 7 1.65 g (yield of 75%) was obtained in the same manner as in the synthesis of Compound 6 of Synthesis Example 1, except that Intermediate 7-3 was used instead of Intermediate 6-3. The obtained compound was identified by Mass spectrum and HPLC analysis.

HRMS (MALDI) calcd for C₆₀H₅₇D₆N₃OPt: m/z 1042.4995, Found: 1042.4996.

### Synthesis Example 8 (Compound 8)

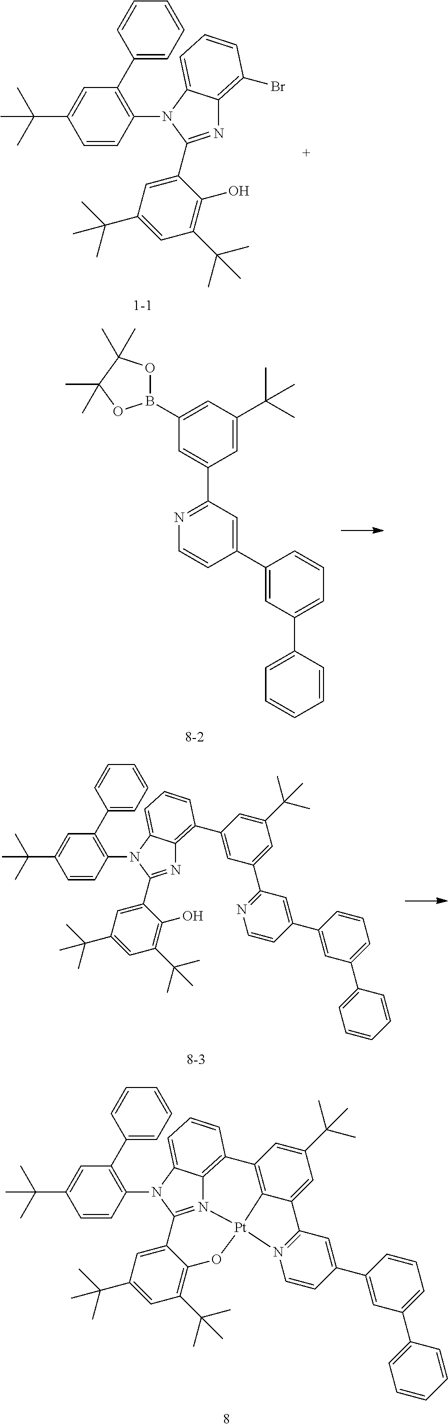

Synthesis of Intermediate 8-3

Intermediate 8-3 1.7 g (yield of 65%) was obtained in the same manner as in the synthesis of Intermediate 6-3 of Synthesis Example 1, except that Intermediate 8-2 was used instead of Intermediate 2-2. The obtained compound was identified by Mass spectrum and HPLC analysis.

HRMS (MALDI) calcd for C₆₄H₆₅N₃O m/z 891.5128, Found: 891.5127.

Synthesis of Compound 8

Compound 8 1.41 g (yield of 68%) was obtained in the same manner as in the synthesis of Compound 6 of Synthesis Example 1, except that Intermediate 8-3 was used instead of Intermediate 6-3. The obtained compound was identified by Mass spectrum and HPLC analysis.

HRMS (MALDI) calcd for C₆₄H₆₃N₃OPt m/z 1084.4619, Found: 1084.4617.

### Synthesis Example 9 (Compound 9)

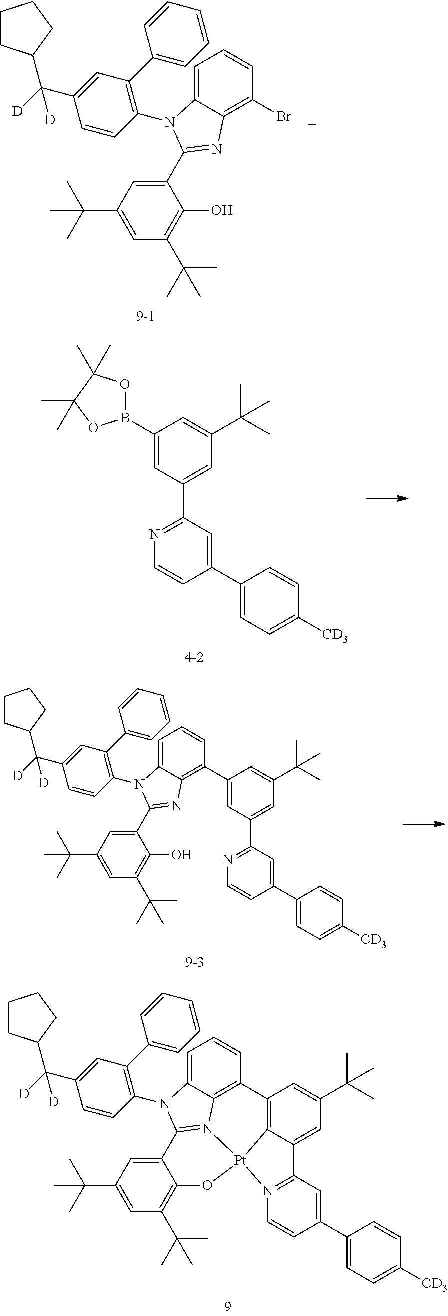

Synthesis of Intermediate 9-3

Intermediate 9-3 2.1 g (yield of 63%) was obtained in the same manner as in the synthesis of Intermediate 4-3 of Synthesis Example 5, except that Intermediate 9-1 was used instead of Intermediate 1-1. The obtained compound was identified by Mass spectrum and HPLC analysis.

HRMS (MALDI) calcd for C₆₁H₆₀D₅N₃O m/z 860.5441 Found 860.5443.

Synthesis of Compound 9

Compound 9 1.77 g (yield of 69%) was obtained in the same manner as in the synthesis of Compound 4 of Synthesis Example 5, except that Intermediate 9-3 was used instead of Intermediate 4-3. The obtained compound was identified by Mass spectrum and HPLC analysis.

HRMS (MALDI) calcd for C₆₁H₅₈D₅N₃OPt m/z 1053.4933 Found 1053.4932.

### Evaluation Example 1 Evaluation of Photoluminescence Quantum Yield (PLQY) and Radiative Decay Rate

CBP and Compound 1 were co-deposited at a weight ratio of 9:1 at a vacuum pressure of 10⁻⁷ torr to manufacture a film having a thickness of 40 nanometers (nm).

A PLQY of the film was evaluated by using a Hamamatsu Photonics absolute PL quantum yield measurement system including a xenon light source, a monochromator, a photonic multichannel analyzer, an integrating sphere and employing a PLQY measurement software (Hamamatsu Photonics, Ltd., Shizuoka, Japan), and a PLQY of Compound 1 was confirmed. The results of this study are shown in Table 2.

A PL spectrum of the film was evaluated at room temperature by using a PicoQuant TRPL measurement system FluoTime 300 and a PicoQuant pumping source PLS340 (excitation wavelength=340 nm, spectral width=20 nm), a wavelength of a main peak of the spectrum was determined, PLS340 repeatedly measured the number of photons emitted from the film at the wavelength of the main peak due to a photon pulse (pulse width=500 picoseconds, ps) applied to the film according to time based on time-correlated single photon counting (TCSPC), thereby obtaining a sufficiently fittable TRPL curve. T_{decay}(Ex) (decay time) of the film was obtained by fitting one or more exponential decay functions to the result obtained therefrom, and the radiative decay rate that is a reciprocal of T_{decay}(Ex) was calculated. The results are shown in Table 2. The function used for fitting is expressed by Equation 20, and the greatest value of T_{decay} obtained from each exponential decay function used for fitting was taken as T_{decay}(Ex). At this time, a baseline or background signal curve was obtained by repeating the same measurement once more for the same measurement time as the measurement time for obtaining the TRPL curve in a dark state (a state in which a pumping signal applied to the predetermined film was blocked), and the baseline or background signal curve was used for fitting as a baseline.

$$f ( t ) = {\sum }_{i = 1}^{n} {A}_{i} exp ( - t \text{/} {T}_{decay , i} ) \qquad Equation 20$$

The measurement of the PLQY and the radiative decay time was performed on Compounds 2 to 9, and results thereof are shown in Table 2.

TABLE 2

| Compound | | Radiative decay rate |
|---|---|---|
| No. | PLQY (%) | (s⁻¹) |
| 1 | 0.999 | 4.15 × 10⁵ |
| 2 | 0.992 | 4.09 × 10⁵ |
| 3 | 0.999 | 4.81 × 10⁵ |
| 4 | 0.999 | 4.85 × 10⁵ |
| 5 | 0.999 | 4.99 × 10⁵ |
| 6 | 0.999 | 4.93 × 10⁵ |
| 7 | 0.998 | 4.90 × 10⁵ |
| 8 | 0.999 | 4.83 × 10⁵ |
| 9 | 0.999 | 4.16 × 10⁵ |

*s⁻¹ = reverse seconds

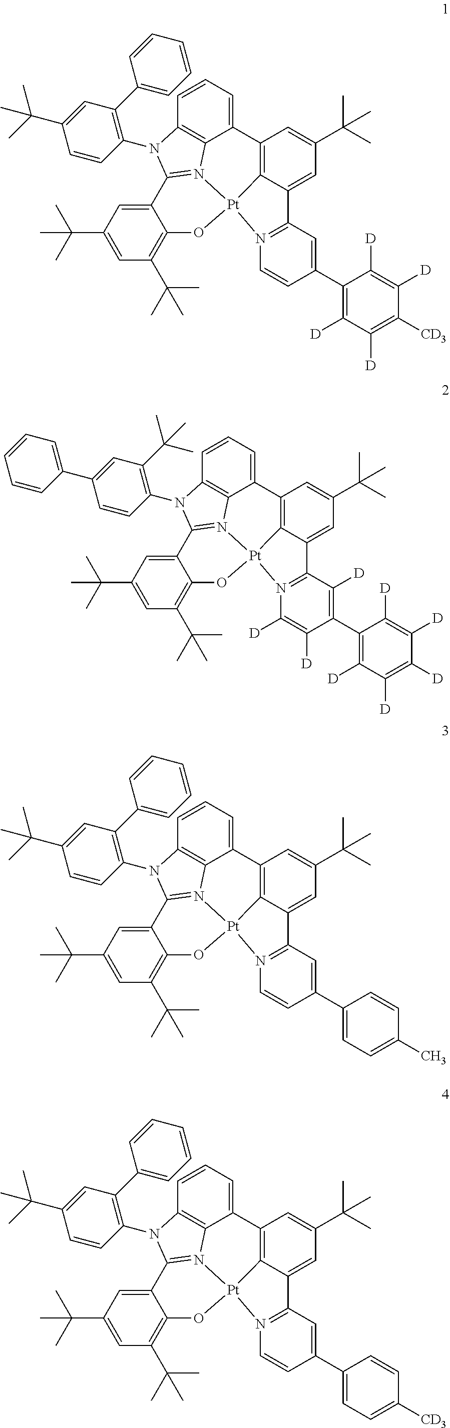

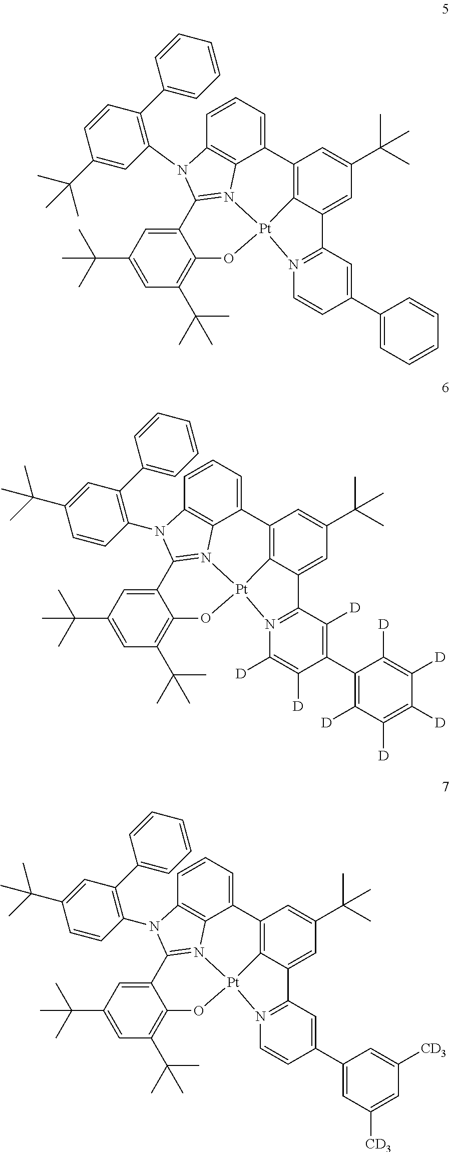

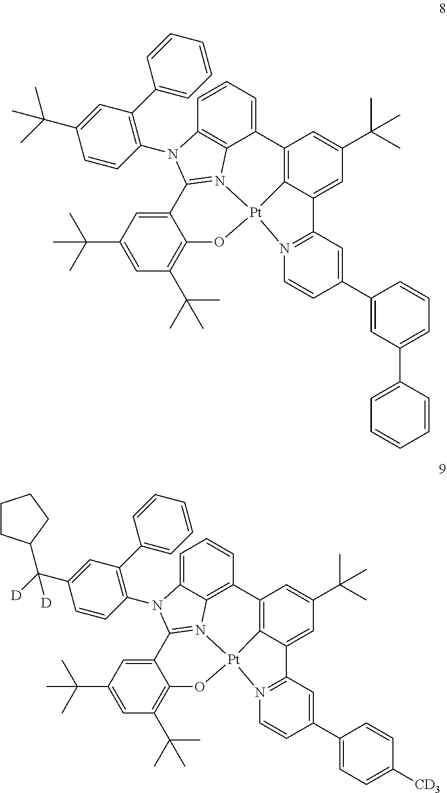

Referring to Table 2, it is confirmed that Compounds 1 to 9 have a high PLQY and a high radiative decay rate.

### Evaluation Example 2: Evaluation of Horizontal Orientation Ratio

mCP and Compound 1 were co-deposited on a fused silica base layer (thickness of 1 mm) at a weight ratio of 92:8 in a vacuum deposition apparatus having a vacuum pressure of 1×10⁻⁷ torr to form Sample 1 having a thickness of 30 nm (8 weight %), and Sample 1 was sealed with glass and glue in a nitrogen atmosphere. This procedure was repeated on Compounds shown in Table 3 to manufacture Samples 2 to 9.

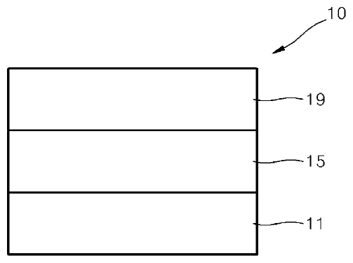

Meanwhile, an angle-dependent PL measurement apparatus having a structure illustrated in FIG. 3 of Korean Patent Application No. 10-2013-0150834, the content of which is incorporated herein in its entirety by reference, was prepared. Detailed specifications are as follows:
Excited light wavelength: 325 nm
Excited light supply source: He—Cd laser, Melles Griot
Excited light irradiation means: Optical fiber, diameter of 1 millimeter (mm), Thorlabs
Semi-cylindrical prism: Fused silica, diameter of 100 mm, length of 30 mm
Emitted light detection means: Photomultiplier tube, Acton
Polarizer mounted on emitted light detection means: Linear polarizer, Thorlabs
Recording apparatus: SpectraSense, Acton
Excited light incidence angle: θP=45°, θH=0°
Distance from sample to emitted light detection means (or radius of moving path of emitted light detection means): 900 mm

Then, each of Samples 1 to 8 was fixed on a semi-cylindrical lens, and a 325-nm laser was irradiated to emit light. The emitted light passed through a polarization film, and p-polarization light emission strength was measured with respect to 530-nm light of 90° to 0° while turning by 1° with respect to the axis of the semi-cylindrical lens, to which the sample was fixed, by using a charge-coupled device (CCD).

p-Polarization light emission strength (first p-polarization light emission strength) appearing when each Compound has a vertical orientation and p-polarization light emission strength (second p-polarization light emission strength) appearing when each Compound had a horizontal orientation were calculated with respect to 0° to 90°. The weights at which p-polarization light emission strength were calculated by multiplying each weight by the first and second p-polarization light emission strengths coinciding with the measured p-polarization light emission strength, and the horizontal orientation ratios of Compounds shown in Table 3 were measured. The results of this study are shown in Table 3. The angle-dependent PL spectrum was analyzed by using a classical dipole moment regarded as dissipated power from a dipole oscillating light emission from excitons.

TABLE 3

| | | Horizontal orientation |
|---|---|---|
| Sample No. | Co-deposition material | ratio (%) |
| 1 | mCP Compound 1 (8 weight %) | 90% |
| 2 | mCP Compound 2 (8 weight %) | 90% |
| 3 | mCP Compound 3 (8 weight %) | 88% |
| 4 | mCP Compound 4 (8 weight %) | 88% |
| 5 | mCP Compound 5 (8 weight %) | 90% |
| 6 | mCP Compound 6 (8 weight %) | 90% |
| 7 | mCP Compound 7 (8 weight %) | 90% |
| 8 | mCP Compound 8 (8 weight %) | 90% |
| 9 | mCP Compound 9 (8 weight %) | 90% |

* weight % = percent by weight

Referring to Table 3, it is confirmed that Compound 1 to 9 have excellent horizontal orientation ratios, that is, excellent horizontal optical orientation.

### Example 1

As an anode, a glass substrate, on which ITO/Ag/ITO (70 Å/1,000 Å/70 Å) were deposited, was cut to a size of 50 mm×50 mm×0.5 mm, sonicated with iso-propyl alcohol and pure water each for 5 minutes, and cleaned by exposure to ultraviolet rays and ozone for 30 minutes. Then, the glass substrate was provided to a vacuum deposition apparatus.

2-TNATA was vacuum-deposited on the anode of the glass substrate to form a hole injection layer having a thickness of 600 Å, and 4,4′-bis[N-(1-naphthyl)-N-phenylamino]biphenyl (NPB) was vacuum-deposited on the hole injection layer to form a hole transport layer having a thickness of 1,350 Å.

CBP (host) and Compound 1 (dopant) were co-deposited on the hole transport layer at a weight ratio of 94:6 to form an emission layer having a thickness of 400 Å.

Then, BCP was vacuum-deposited on the emission layer to form a hole blocking layer having a thickness of 50 Å, Alq₃ was vacuum-deposited on the hole blocking layer to form an electron transport layer having a thickness of 350 Å, LiF was deposited on the electron transport layer to form an electron injection layer having a thickness of 10 Å, and MgAg was deposited on the electron injection layer at a weight ratio of 90:10 to form a cathode having a thickness of 120 Å, thereby completing an organic light-emitting device having a structure of anode/2-TNATA (600 Å)/NPB (1,350 Å)/CBP+Compound 1 (6 weight %) (400 Å)/BCP (50 Å)/Alq₃ (350 Å) /LiF (10 Å)/MgAg (120 Å).

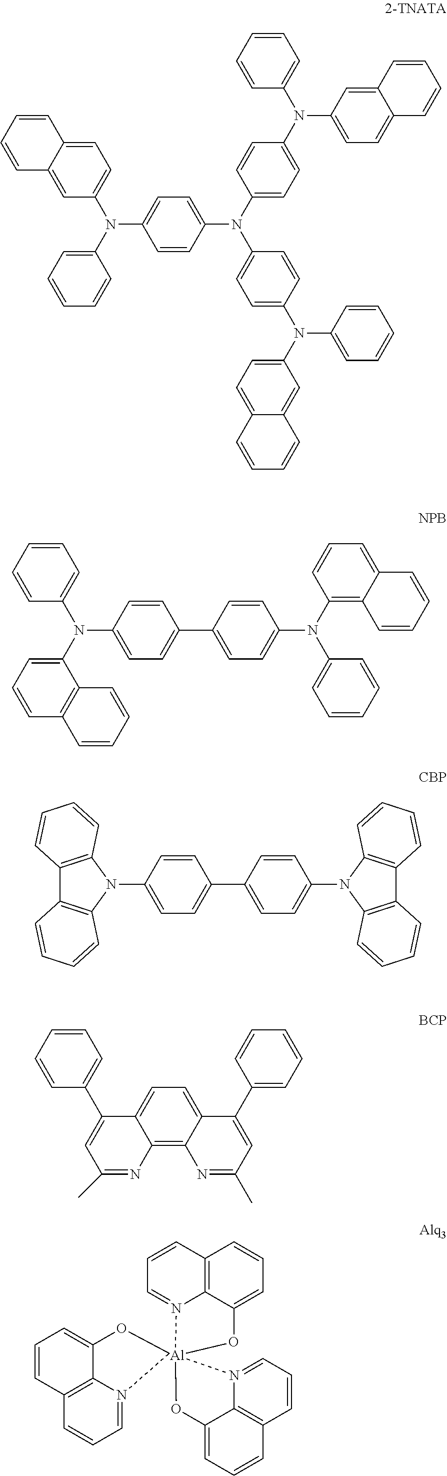

### Examples 2 to 9 and Comparative Examples a to E

Organic light-emitting devices were manufactured in the same manner as in Example 1, except that Compound shown in Table 4 were each used instead of Compound 1 as a dopant in forming an emission layer.

### Evaluation Example 3: Evaluation of Characteristics of Organic Light-Emitting Devices

The driving voltage, current density, maximum quantum efficiency, roll-off ratio, full width at half maximum (FWHM), peak emission wavelength, and lifespan of the organic light-emitting devices manufactured according to Examples 1 to 9 and Comparative Example A to E were evaluated by using a current-voltage meter (Keithley 2400) and a luminance meter (Minolta Cs-1000A), and results thereof are shown in Tables 4 and 5. The roll-off ratio was calculated by Equation 30. The lifespan (LT₉₉, at 3,500 nit) indicates an amount of time that elapsed when luminance was 99% of initial luminance (100%) and is expressed by a relative value (%).

roll off ratio={1−(efficiency (at 3,500 nit)/maximum luminescent efficiency)}×100% Equation 30

TABLE 4

| | | | | Maximum | Roll- | |
|---|---|---|---|---|---|---|
| | Dopant | Driving | Current | quantum | off | |
| | compound | voltage | density | efficiency | ratio | FWHM |
| | No. | (V) | (mA/cm²) | (%) | (%) | (nm) |
| Example 1 | 1 | 3.69 | 10 | 112 | 9 | 68 |
| Example 2 | 2 | 3.72 | 10 | 102 | 6 | 68 |
| Example 3 | 3 | 3.79 | 10 | 107 | 8 | 68 |
| Example 4 | 4 | 3.79 | 10 | 109 | 9 | 67 |
| Example 5 | 5 | 3.83 | 10 | 99 | 7 | 69 |
| Example 6 | 6 | 4.60 | 10 | 108 | 10 | 70 |
| Example 7 | 7 | 3.84 | 10 | 111 | 8 | 67 |
| Example 8 | 8 | 3.80 | 10 | 114 | 7 | 67 |
| Example 9 | 9 | 3.96 | 10 | 110 | 7 | 67 |
| Comparative | A | 3.94 | 10 | 94 | 10 | 68 |
| Example A | | | | | | |
| Comparative | B | 4.08 | 10 | 93 | 8 | 68 |
| Example B | | | | | | |
| Comparative | C | 3.85 | 10 | 98 | 4 | 68 |
| Example C | | | | | | |
| Comparative | D | 3.90 | 10 | 88 | 8 | 67 |
| Example D | | | | | | |
| Comparative | E | 4.39 | 10 | 88 | 17 | 64 |
| Example E | | | | | | |

TABLE 5

| | Dopant | Peak emission | Lifespan (LT₉₉) |
|---|---|---|---|
| | compound | wavelength | (at 3,500 nit) |
| | No. | (nm) | a relative value (%) |
| Example 1 | 1 | 521 | 115% |
| Example 2 | 2 | 523 | 110% |
| Example 3 | 3 | 521 | 113% |
| Example 4 | 4 | 521 | 115% |
| Example 5 | 5 | 526 | 110% |
| Example 6 | 6 | 525 | 130% |
| Example 7 | 7 | 524 | 110% |
| Example 8 | 8 | 527 | 180% |
| Example 9 | 9 | 523 | 105% |
| Comparative Example | A | 523 | 29% |
| A | | | |
| Comparative Example | B | 526 | 50% |
| B | | | |
| Comparative Example | C | 523 | 60% |
| C | | | |
| Comparative Example | D | 520 | 3% |
| D | | | |
| Comparative Example | E | 502 | 1% |
| E | | | |

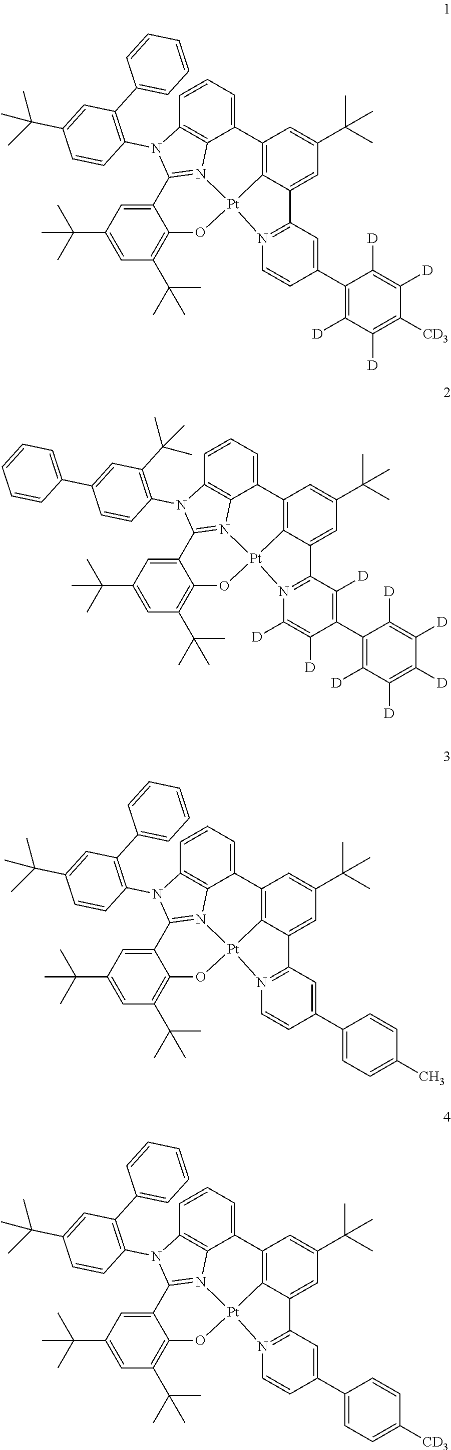

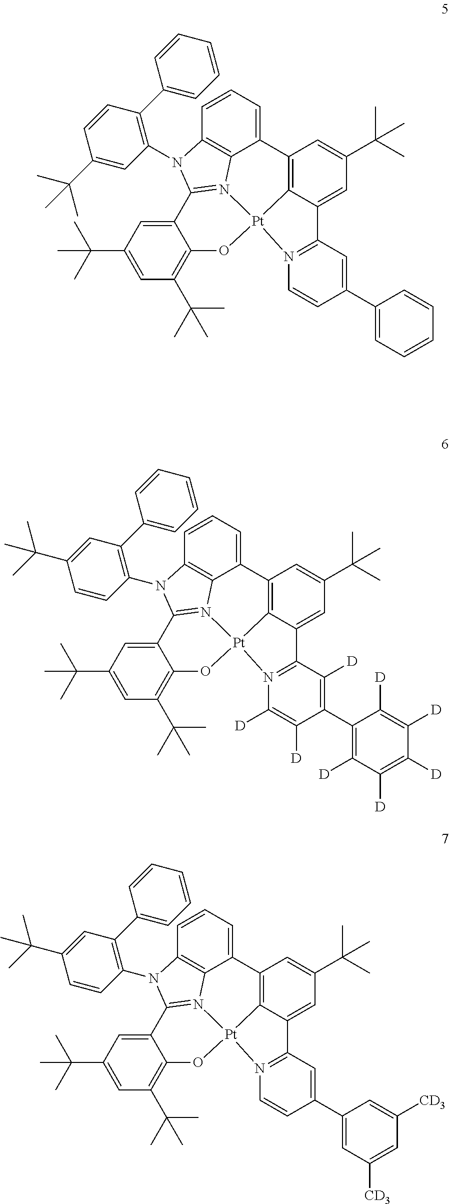

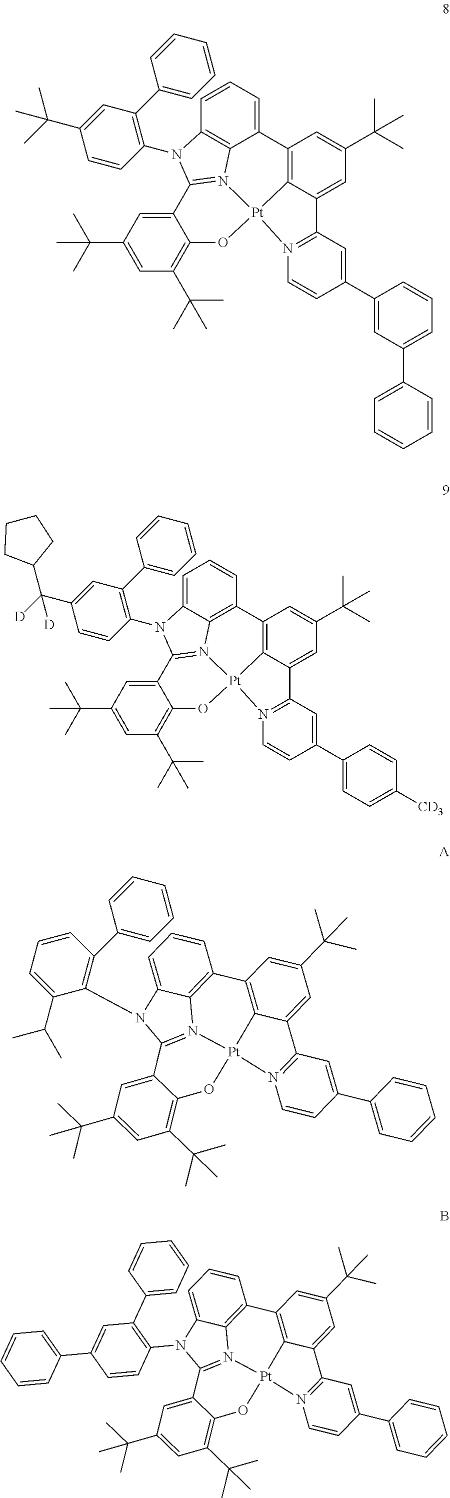

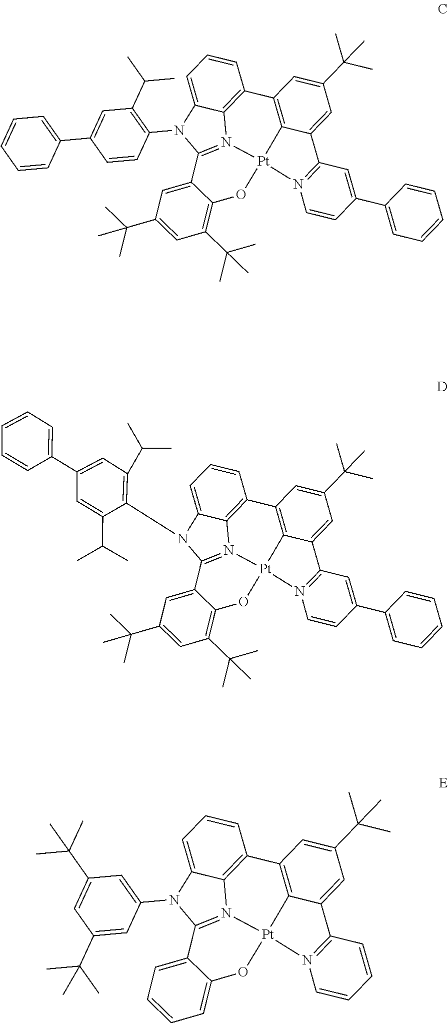

Referring to Tables 4 and 5, it is confirmed that the organic light-emitting devices of Examples 1 to 9 have improved driving voltage, improved maximum quantum efficiency, and/or improved roll-off ratio, and also have improved lifespan characteristics, as compared with those of the organic light-emitting devices of Comparative Examples A to E.

Since the organometallic compound may emit light having a relatively small FWHM and have excellent PLQY, excellent radiative decay rate, and excellent horizontal orientation ratio, the organic light-emitting device including the organometallic compound may have improved driving voltage, external quantum efficiency, roll-off ratio, and lifespan characteristics. In addition, since the organometallic compound has excellent phosphorescence characteristics, a diagnostic composition including the organometallic compound may have high diagnostic efficiency.

It should be understood that embodiments described herein should be considered in a descriptive sense only and not for purposes of limitation. Descriptions of features or aspects within each embodiment should typically be considered as available for other similar features or aspects in other embodiments.

While embodiments have been described with reference to the figures, it will be understood by those of ordinary skill in the art that various changes in form and details may be made therein without departing from the spirit and scope of the present description as defined by the following claims.

What is claimed is:

1. An organometallic compound represented by Formula 1: 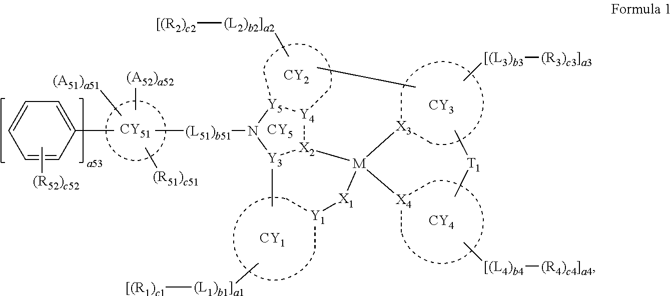
wherein, in Formula 1,
M is a transition metal,
X₁ is O or S, wherein a bond between X₁ and M is a covalent bond,
X₂ to X₄ are each independently C or N,
a bond between X₂ and M, a bond between X₃ and M, or a bond between X₄ and M is a covalent bond, and the other bonds of a bond between X₂ and M, a bond between X₃ and M, and a bond between X₄ and M are coordinate bonds,
Y₁ and Y₃ to Y₅ are each independently C or N,
a bond between X₂ and Y₃, a bond between X₂ and Y₄, and a bond between Y₄ and Y₅ is a chemical bond,
ring CY₁ to ring CY₄ and ring CY₅₁ are each independently a C₅-C₃₀ carbocyclic group or a C₂-C₃₀ heterocyclic group,
a cyclometalated ring formed by ring CY₅, ring CY₂, ring CY₃, and M is a 6-membered ring,
T₁ is a single bond, a double bond, *—N(R₅)—*′, *—B(R₅)—*′, *—P(R₅)—*′, *—C(R₅)(R₆)—*′, *—Si(R₅)(R₆)—*′, *—Ge(R₅)(R₆)—*′, *—S—*, *—Se—*′, * *, *—C(═O)—*′, *—S(═O)—*′, *—S(═O)₂—*, * C(R₅)=*′, *═C(R₅)—*′, *—C(R₅)═C(R₆)—*′, *—C(═S)—*′, or *—C≡C—*′, wherein * and *′ each indicate a binding site to a neighboring atom,
L₁ to L₄ and L₅₁ are each independently a single bond, a C₅-C₃₀ carbocyclic group that is unsubstituted or substituted with an R₁₀ₐ group, or a C₂-C₃₀ heterocyclic group that is unsubstituted or substituted with an R₁₀ₐ group,
b1 to b4 and b51 are each independently an integer from 1 to 5,
R₁ to R₆, R₅₁, and R₅₂ are each independently hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₂-C₁₀ heterocycloalkyl group, a substituted or unsubstituted C₃-C₁₀ cycloalkenyl group, a substituted or unsubstituted C₂-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₇-C₆₀ arylalkyl group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted C₂-C₆₀ heteroarylalkyl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉),
c1 to c4, c51 and c52 are each independently an integer from 1 to 5,
A₅₁ is a C₄-C₆₀ alkyl group,
A₅₂ is deuterium or a deuterium-containing C₁-C₆₀ alkyl group unsubstituted or substituted with a C₃-C₁₀ cycloalkyl group,
a1 to a4, a51, and a52 are each independently an integer from 0 to 10, provided that the sum of a51 and a52 is 1 or more,
a53 is an integer from 1 to 10,
two or more groups of a plurality of R₁ groups are optionally linked to form a C₅-C₃₀ carbocyclic group that is unsubstituted or substituted with an R₁₀ₐ group or a C₂-C₃₀ heterocyclic group that is unsubstituted or substituted with an R₁₀ₐ group,
two or more groups of a plurality of R₂ groups are optionally linked to form a C₅-C₃₀ carbocyclic group that is unsubstituted or substituted with an R₁₀ₐ group or a C₂-C₃₀ heterocyclic group that is unsubstituted or substituted with an R₁₀ₐ group,
two or more groups of a plurality of R₃ groups are optionally linked to form a C₅-C₃₀ carbocyclic group that is unsubstituted or substituted with an R₁₀ₐ group or a C₂-C₃₀ heterocyclic group that is unsubstituted or substituted with an R₁₀ₐ group,
two or more groups of a plurality of R₄ groups are optionally linked to form a C₅-C₃₀ carbocyclic group that is unsubstituted or substituted with an an R₁₀ₐ group or a C₂-C₃₀ heterocyclic group that is unsubstituted or substituted with an R₁₀ₐ group,
two or more groups of R₁ to R₆ are optionally linked to form a C₅-C₃₀ carbocyclic group that is unsubstituted or substituted with an R₁₀ₐ group or a C₂-C₃₀ heterocyclic group that is unsubstituted or substituted with an R₁₀ₐ group,
R₁₀ₐ is the same as described in connection with R₁,
a substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₂-C₁₀ heterocycloalkyl group, the substituted C₃-C₁₀ cycloalkenyl group, the substituted C₂-C₁₀ heterocycloalkenyl group, the substituted C₆-C₆₀ aryl group, the substituted C₆-C₆₀ aryloxy group, the substituted C₆-C₆₀ arylthio group, the substituted C₇-C₆₀ arylalkyl group, the substituted C₁-C₆₀ heteroaryl group, the substituted C₇-C₆₀ arylalkyl group, the substituted C₁-C₆₀ heteroaryl group, the substituted C₁-C₆₀ heteroaryloxy group, the substituted C₁-C₆₀ heteroarylthio group, the substituted C₂-C₆₀ heteroarylalkyl group, the substituted monovalent non-aromatic condensed polycyclic group, and the substituted monovalent non-aromatic condensed heteropolycyclic group is:
deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or any combination thereof;
a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, and a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₂-C₁₀ heterocycloalkyl group, a C₃-C₁₀ cycloalkenyl group, a C₂-C₁₀ heterocycloalkenyl group, a C₆-C₆₀ aryl group, a C₆-C₆₀ aryloxy group, a C₆-C₆₀ arylthio group, a C₇-C₆₀ arylalkyl group, a C₁-C₆₀ heteroaryl group, a C₁-C₆₀ heteroaryloxy group, a C₁-C₆₀ heteroarylthio group, a C₂-C₆₀ heteroarylalkyl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or any combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or
any combination thereof; and
Q₁ to Q₉, Q₁₁ to Q₁₉, Q₂₁ to Q₂₉, and Q₃₁ to Q₃₉ are each independently hydrogen; deuterium; —F; —Cl; —Br; —I; a hydroxyl group; a cyano group; a nitro group; an amidino group; a hydrazine group; a hydrazone group; a carboxylic acid group or a salt thereof; a sulfonic acid group or a salt thereof; a phosphoric acid group or a salt thereof; a C₁-C₆₀ alkyl group unsubstituted or substituted with deuterium, a C₁-C₆₀ alkyl group, a C₆-C₆₀ aryl group, or any combination thereof; a C₂-C₆₀ alkenyl group; a C₂-C₆₀ alkynyl group; a C₁-C₆₀ alkoxy group; a C₃-C₁₀ cycloalkyl group; a C₂-C₁₀ heterocycloalkyl group; a C₃-C₁₀ cycloalkenyl group; a C₂-C₁₀ heterocycloalkenyl group; a C₆-C₆₀ aryl group unsubstituted or substituted with deuterium, a C₁-C₆₀ alkyl group, a C₆-C₆₀ aryl group, or any combination thereof; a C₆-C₆₀ aryloxy group; a C₆-C₆₀ arylthio group; a C₇-C₆₀ arylalkyl group; a C₁-C₆₀ heteroaryl group; a C₁-C₆₀ heteroaryloxy group; a C₁-C₆₀ heteroarylthio group; a C₂-C₆₀ heteroarylalkyl group; a monovalent non-aromatic condensed polycyclic group; or a monovalent non-aromatic condensed heteropolycyclic group.

2. The organometallic compound of claim 1, wherein
M is Pt, Pd, or Au.

3. The organometallic compound of claim 1, wherein
A₅₁ is an n-butyl group, a sec-butyl group, an iso-butyl group, a tert-butyl group, an n-pentyl group, a tert-pentyl group, a neo-pentyl group, an iso-pentyl group, a sec-pentyl group, a 3-pentyl group, or a sec-iso-pentyl group, unsubstituted or substituted with a methyl group, an ethyl group, an n-propyl group, an iso-propyl group, an n-butyl group, a sec-butyl group, an iso-butyl group, a tert-butyl group, an n-pentyl group, a tert-pentyl group, a neo-pentyl group, an iso-pentyl group, a sec-pentyl group, a 3-pentyl group, a sec-iso-pentyl group, or any combination thereof, and
A₅₂ is a deuterium-containing linear or branched C₁-C₂₀ alkyl group unsubstituted or substituted with a C₃-C₁₀ cycloalkyl group, in which the linear or branched C₁-C₂₀ alkyl group is a methyl group, an ethyl group, an n-propyl group, an iso-propyl group, an n-butyl group, a sec-butyl group, an iso-butyl group, a tert-butyl group, an n-pentyl group, a tert-pentyl group, a neo-pentyl group, an iso-pentyl group, a sec-pentyl group, a 3-pentyl group, or a sec-iso-pentyl group, each unsubstituted or substituted with a methyl group, an ethyl group, an n-propyl group, an iso-propyl group, an n-butyl group, a sec-butyl group, an iso-butyl group, a tert-butyl group, an n-pentyl group, a tert-pentyl group, a neo-pentyl group, an iso-pentyl group, a sec-pentyl group, a 3-pentyl group, a sec-iso-pentyl group, or any combination thereof.

4. The organometallic compound of claim 1, wherein
a3 and a4 are not 0, and
a group represented by *-(L₃)_{b3}-(R₃)_{c3}, and a group represented by *-(L₄)_{b4}-(R₄)_{c4} are not hydrogen.

5. The organometallic compound of claim 1, wherein
a3 is not 0, and
an *-(L₃)_{b3}-(R₃)_{c3} in a number of a3 satisfies Condition A and Condition B:
Condition A
L₃ is a single bond,
Condition B
R₃ is
hydrogen, deuterium, —F, a cyano group, C₁-C₂₀ alkyl group, or a C₁-C₂₀ alkoxy group;
a C₁-C₂₀ alkyl group or a C₁-C₂₀ alkoxy group, each substituted with deuterium, —F, —CD₃, —CD₂H, —CDH₂, —CF₃, —CF₂H, —CFH₂, a cyano group, a C₁-C₁₀ alkyl group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof; or
a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a phenyl group, a (C₁-C₂₀ alkyl)phenyl group, a biphenyl group, or a terphenyl group, each unsubstituted or substituted with deuterium, —F, —CD₃, —CD₂H, —CDH₂, —CF₃, —CF₂H, —CFH₂, a cyano group, a C₁-C₂₀ alkyl group, a deuterium-containing C₂-C₂₀ alkyl group, a C₁-C₂₀ alkoxy group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof.

6. The organometallic compound of claim 1, wherein
a4 is not 0, and
a group represented by *-(L₄)_{b4}-(R₄)_{c4} in a number of a4 satisfies Condition 1, Condition 2, or combination thereof:
Condition 1
An R₄ in a number of c4 is a substituted or unsubstituted C₆-C₆₀ aryl group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group,
Condition 2
L₄ is not a single bond.

7. The organometallic compound of claim 1, wherein
a4 is not 0, and
an *-(L₄)_{b4}-(R₄)_{c4} in a number of a4 satisfies Condition 1(1), Condition 2(1), or combination thereof:
Condition 1(1)
An R₄ in a number of c4 is a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a phenyl group, a (C₁-C₂₀ alkyl)phenyl group, a biphenyl group, or a terphenyl group, each unsubstituted or substituted with deuterium, —F, —CD₃, —CD₂H, —CDH₂, —CF₃, —CF₂H, —CFH₂, a cyano group, a C₁-C₂₀ alkyl group, a deuterium-containing C₂-C₂₀ alkyl group, a C₁-C₂₀ alkoxy group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof,
Condition 2(1)
L₄ is a benzene group unsubstituted or substituted with deuterium, —F, —CD₃, —CD₂H, —CDH₂, —CF₃, —CF₂H, —CFH₂, a cyano group, a C₁-C₂₀ alkyl group, a deuterium-containing C₂-C₂₀ alkyl group, a C₁-C₂₀ alkoxy group, a cyclopentyl group, a cyclohexyl group, a cycloheptyl group, a cyclooctyl group, an adamantyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a phenyl group, a (C₁-C₂₀ alkyl)phenyl group, a biphenyl group, a terphenyl group, or any combination thereof.

8. The organometallic compound of claim 1, wherein
a4 is not 0, and
an *-(L₄)_{b4}-(R₄)_{c4} in a number of a4 satisfies Condition 3, Condition 4, Condition 5, or combination thereof:
Condition 3
R₄ in a number of c4 is a substituted C₆-C₆₀ aryl group,
Condition 4
L₄ is a C₅-C₃₀ carbocyclic group substituted with an R₁₀ₐ group,
Condition 5
L₄ is a C₅-C₃₀ carbocyclic group that is unsubstituted or substituted with an R₁₀ₐ group and R₄ is not hydrogen.

9. The organometallic compound of claim 1, wherein
a51 and a52 are each independently 0, 1, or 2,
the sum of a51 and a52 is 1 or 2, and
a53 is 1 or 2.

10. The organometallic compound of claim 1, wherein,
in Formula 1,
a group represented by 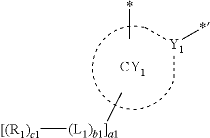
is a group represented by one of Formulae CY1(1) to CY1(8),
a group represented by 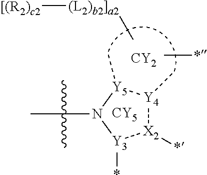
is a group represented by one of Formulae CY2(1) to CY2(4),
a group represented by 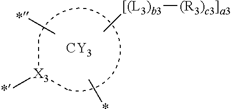
is a group represented by one of Formulae CY3(1) to CY3(24), and
a group represented by 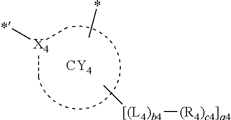
is a group represented by one of Formulae CY4(1) to CY4(74): 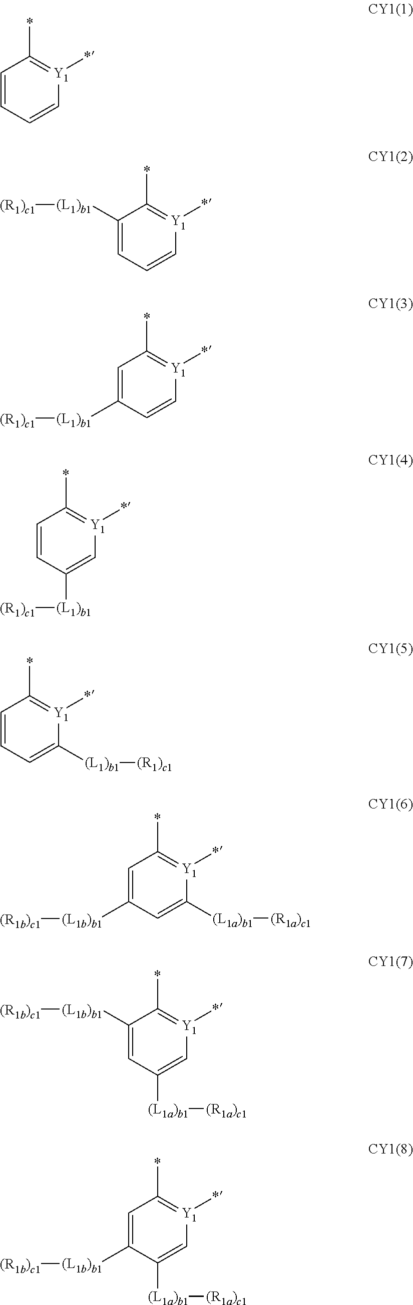 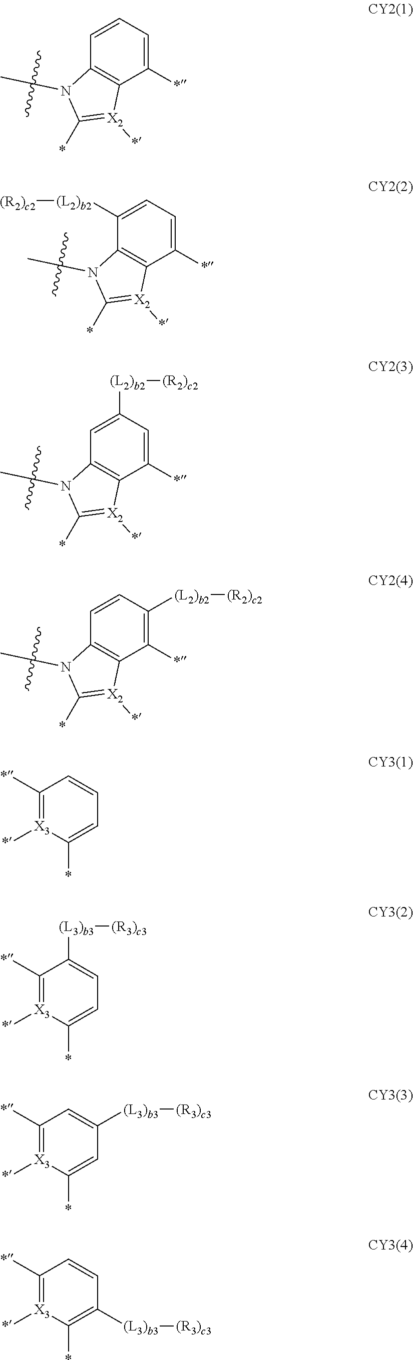 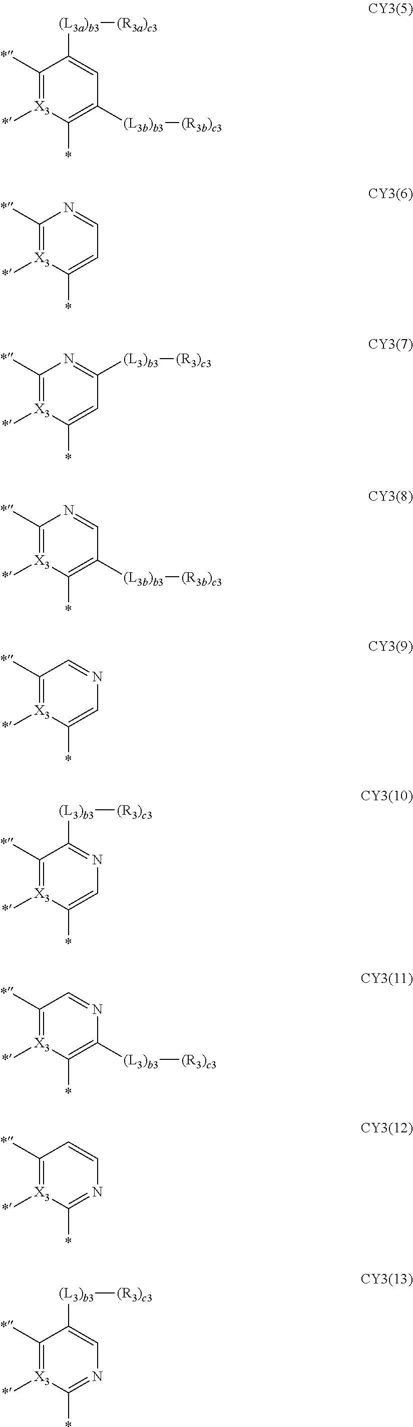 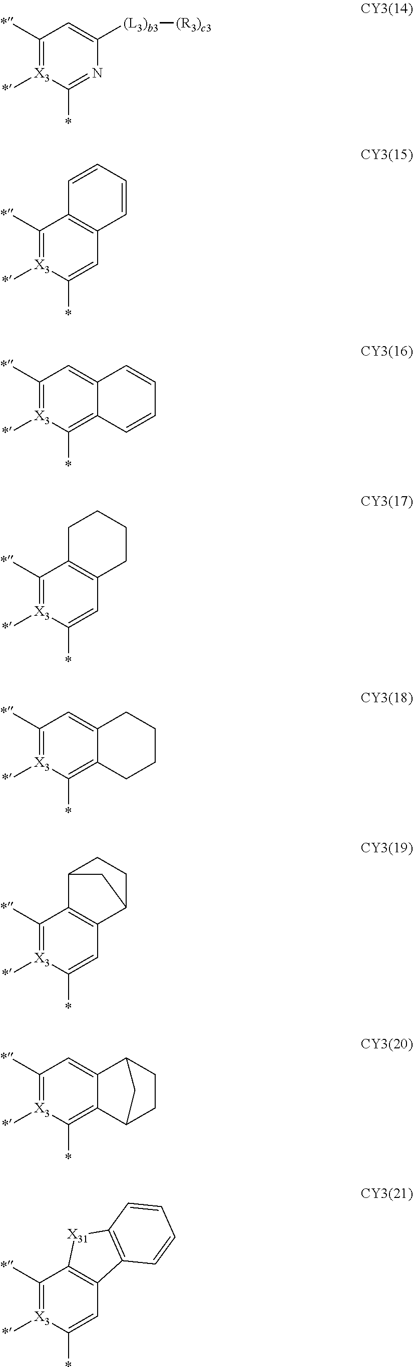 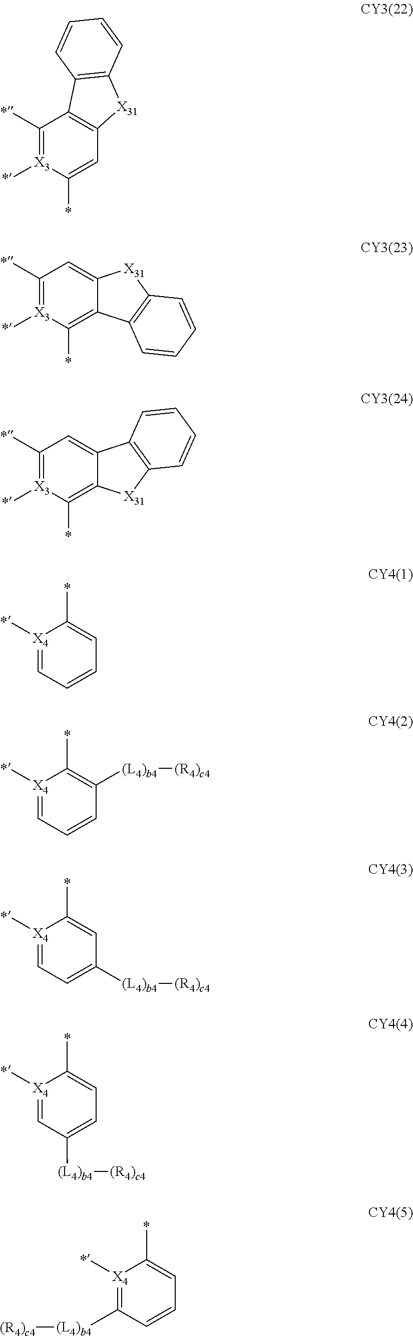 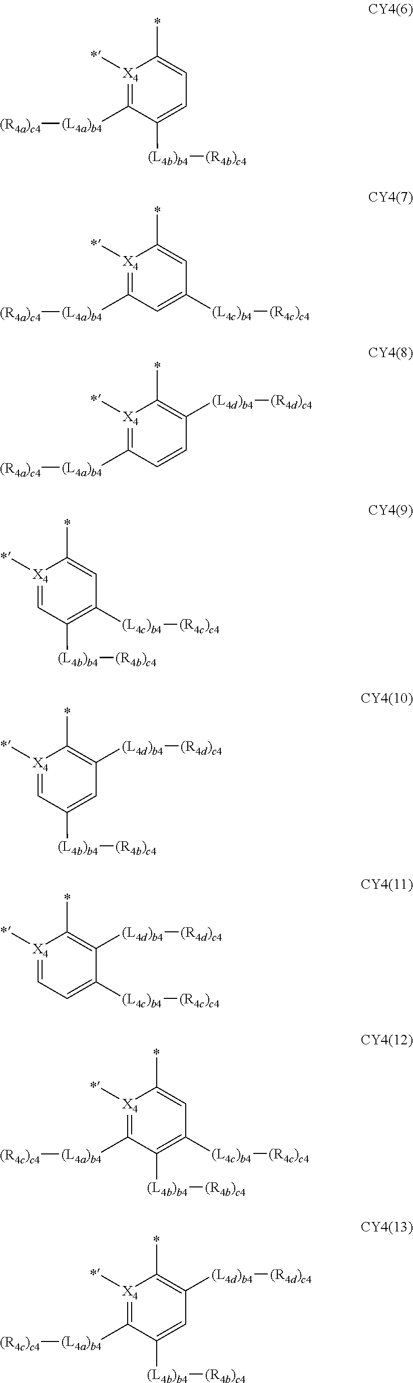 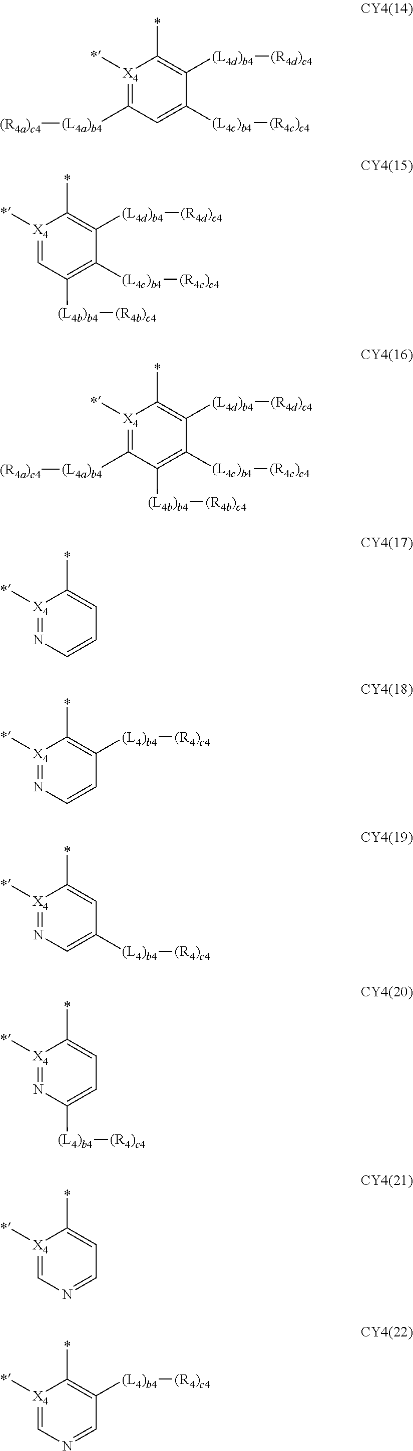 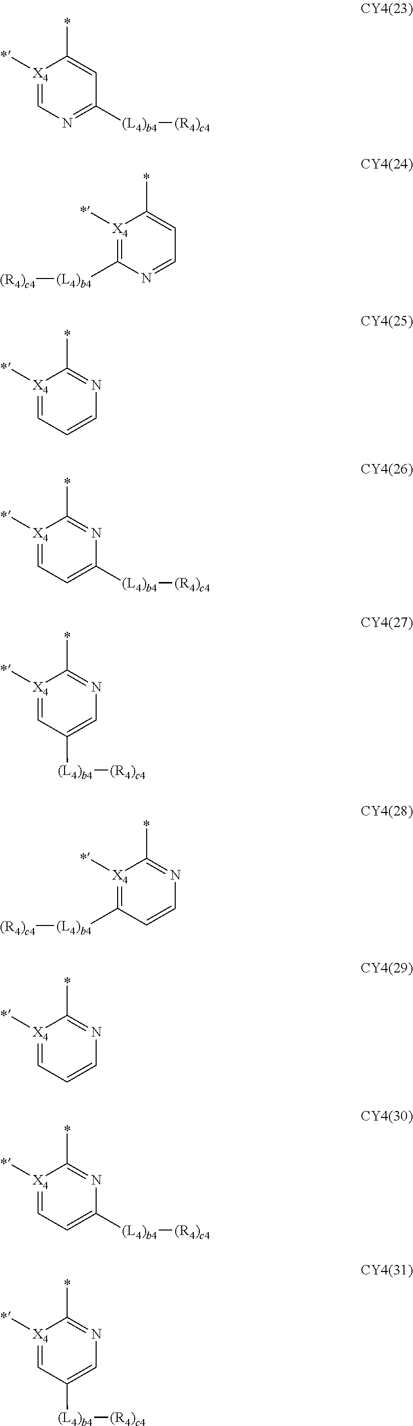 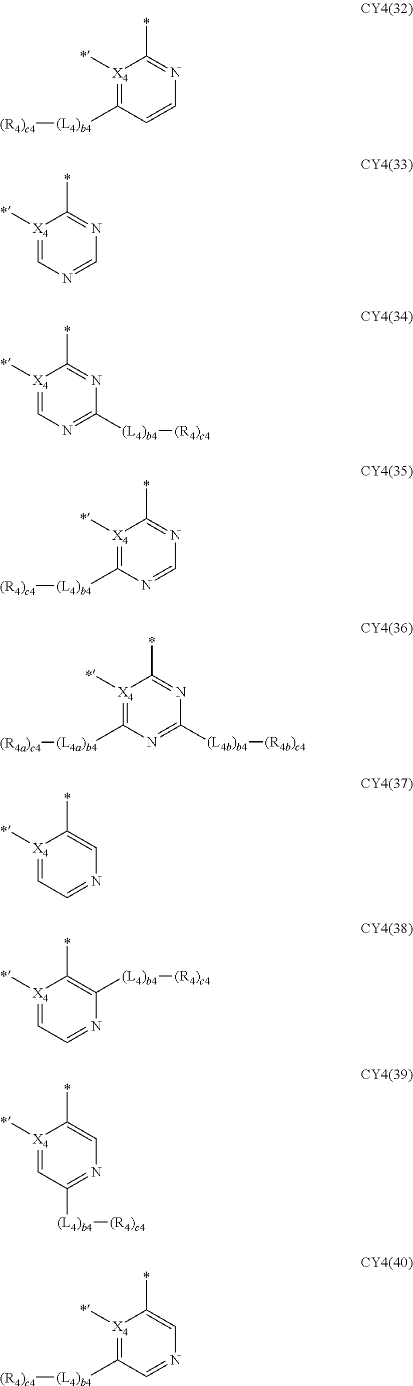 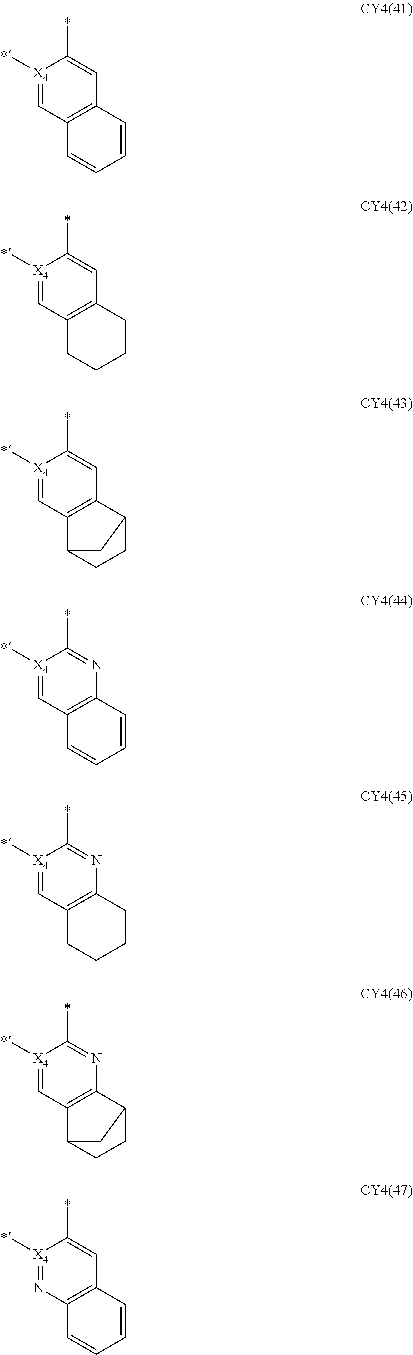 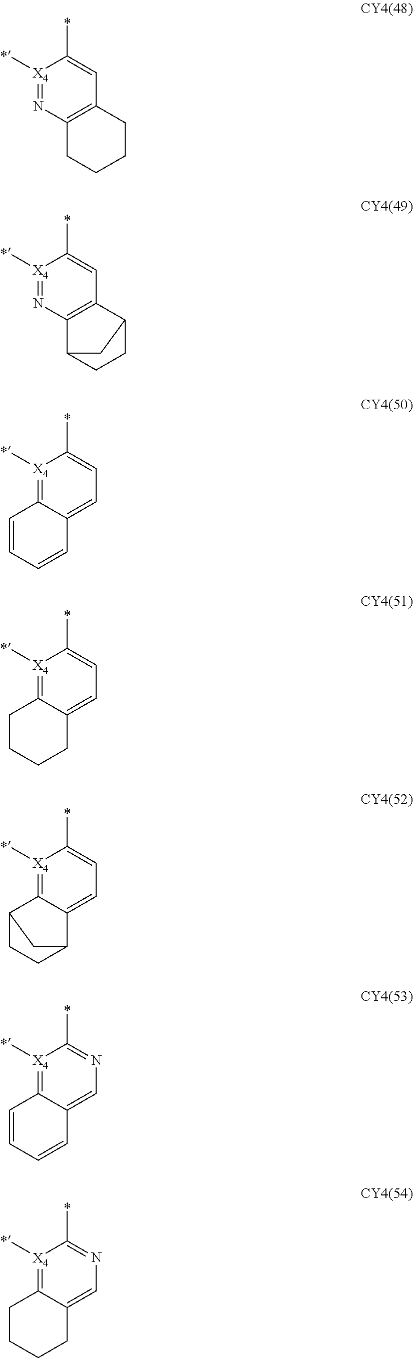 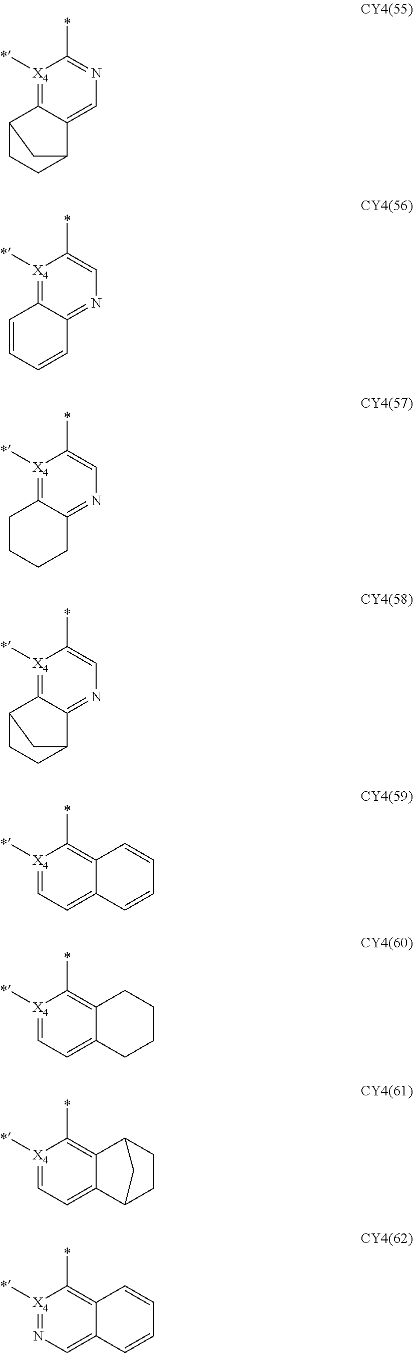 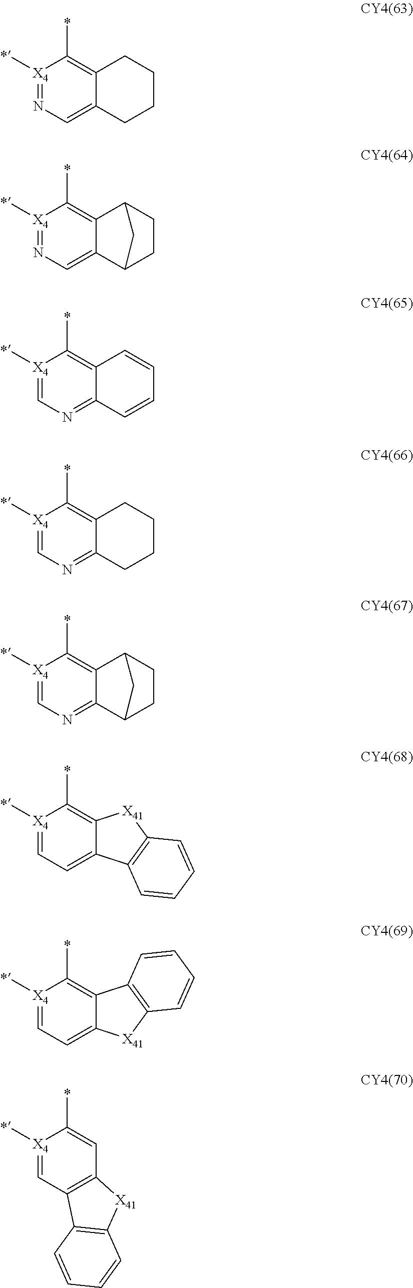 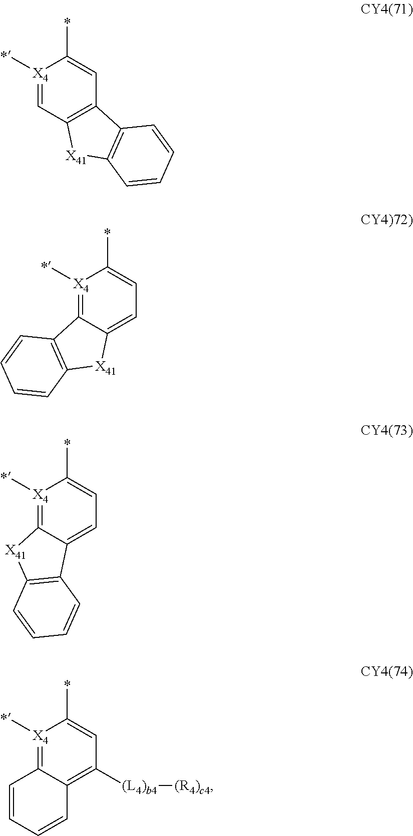
wherein, in Formulae CY1(1) to CY1(8), CY2(1) to CY2(4), CY3(1) to CY3(24), and CY4(1) to CY4(74),
X₂ to X₄, Y₁, L₁ to L₄, b1 to b4, R₁ to R₄, and c1 to c4 are each independently the same as described in claim 1,
X₃₁ is O, S, N(R₃₁), C(R₃₁)(R₃₂), or Si(R₃₁)(R₃₂),
X₄₁ is O, S, N(R₄₁), C(R₄₁)(R₄₂), or Si(R₄₁)(R₄₂),
L₁ₐ and L_{1b} are each independently the same as described in connection with L₁ in claim 1,
R₁ₐ and R_{1b} are each independently the same as described in connection with R₁ in claim 1,
L₃ₐ and L_{3b} are each independently the same as described in connection with L₃ in claim 1,
R₃ₐ, R_{3b}, R₃₁, and R₃₂ are each independently the same as described in connection with R₃ in claim 1,
L₄ₐ to L_{4d} are each independently the same as described in connection with L₄ in claim 1,
R₄ₐ to R_{4d}, R₄₁, and R₄₂ are each independently the same as described in connection with R₄ in claim 1,
*-(L₁)_{b1}-(R₁)_{c1}, *-(L₁ₐ)_{b1}-(R₁ₐ)_{c1}, *-(L_{1b})_{b1}-(R₁ₐ)_{c1}, *-(L₂)_{b2}-(R₂)_{c2}, *-(L₃)_{b3}-(R₃)_{c3}, * (L₃ₐ)_{b3}-(R₃ₐ)_{c3}, *-(L_{3b})_{b3}-(R₃ₐ)_{c3}, *-(L₄)_{b4}-(R₄)_{c4}, *-(L₄ₐ)_{b4}-(R₄ₐ)_{c4}, *-(L_{4b})_{b4}-(R₄ₐ)_{c4}, *-(L_{4c})_{b4}-(R_{4c})_{c4}, and *-(L_{4d})_{b4}-(R_{4d})_{c4} are not hydrogen, wherein * indicates a binding site to a neighboring atom,
in Formulae CY1(1) to CY1(8), *′ indicates a binding site to X₁ in Formula 1,
in Formulae CY2(1) to CY2(4), CY3(1) to CY3(24), and CY4(1) to CY4(74), *′ indicates a binding site to M in Formula 1,
in Formulae CY1(1) to CY1(8), * indicates a binding site to Y₃ in Formula 1,
in Formulae CY2(1) to CY2(4), * indicates a binding site to ring CY1 in Formula 1, and *″ indicates a binding site to ring CY3 in Formula 1,
in Formulae CY3(1) to CY3(24), *″ indicates a binding site to ring CY2 in Formula 1, and * indicates a binding site to T₁ in Formula 1, and
in Formulae CY4(1) to CY4(74), * indicates a binding site to T₁ in Formula 1.

11. The organometallic compound of claim 1, wherein,
in Formula 1,
a group represented by 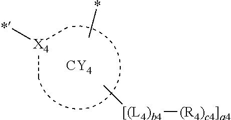
is a group represented by Formula CY4-1 or CY4-2: 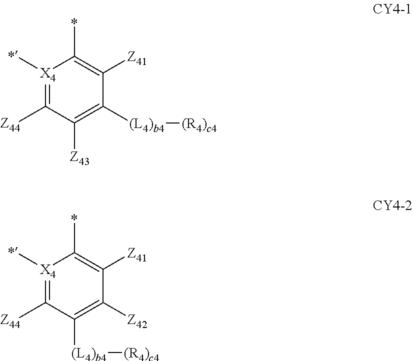
wherein, in Formulae CY4-1 and CY4-2,
X₄, L₄, b4, R₄, and c4 are each independently the same as described in claim 1,
Z₄₁ to Z₄₄ are each independently the same as described in connection with R₄ in claim 1,
′ indicates a binding site to M in Formula 1, and
indicates a binding site to T₁ in Formula 1.

12. The organometallic compound of claim 10, wherein,
in Formula 1,
a group represented by 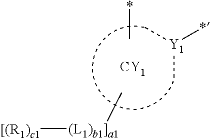
is a group represented by Formula CY1 (1) or CY1 (6),
a group represented by 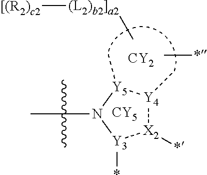
is a group represented by Formula CY2(1),
a group represented by 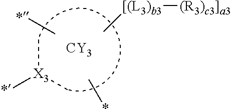
is a group represented by Formula CY3(3), and
a group represented by 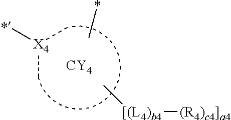
is a group represented by Formula CY4(3), CY4(4) or CY4(16).

13. The organometallic compound of claim 1, wherein
a group represented by 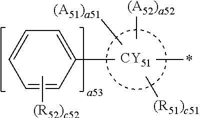
in Formula 1 is a group represented by one of Formulae 51-1 to 51-32: 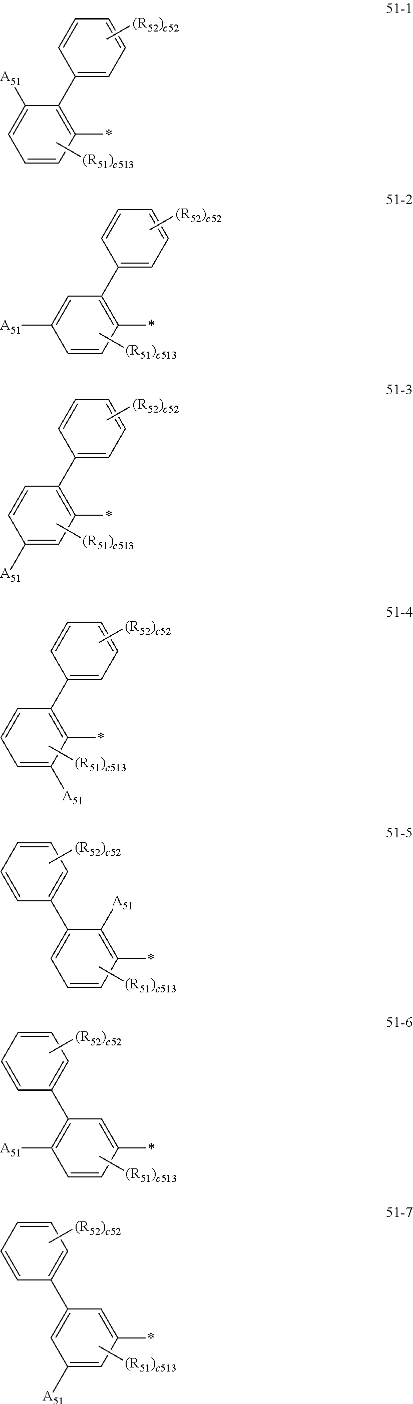 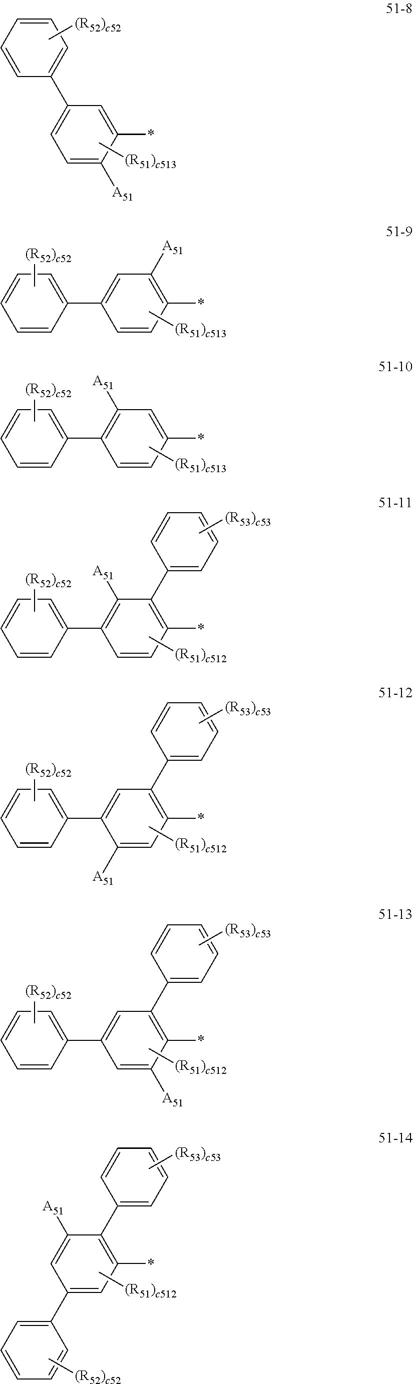 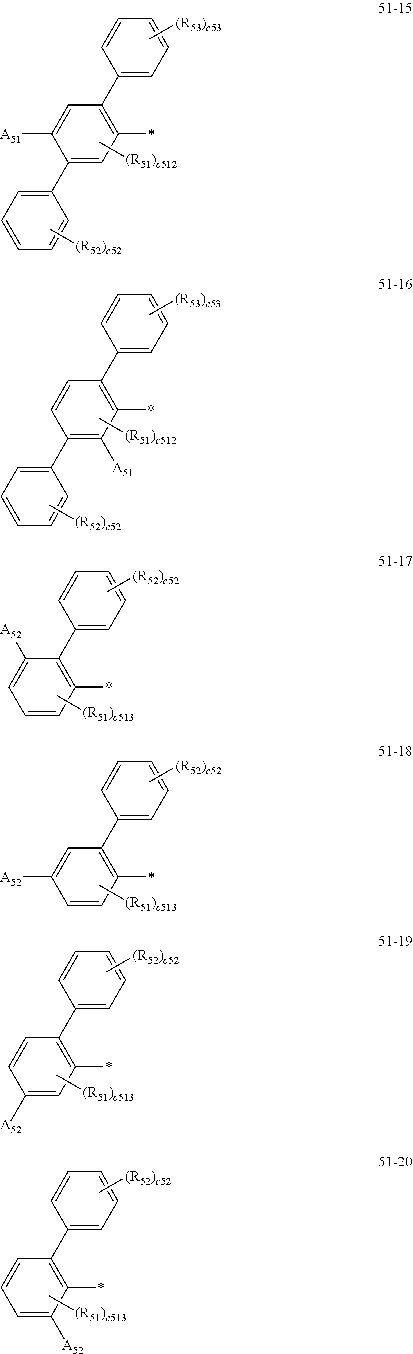 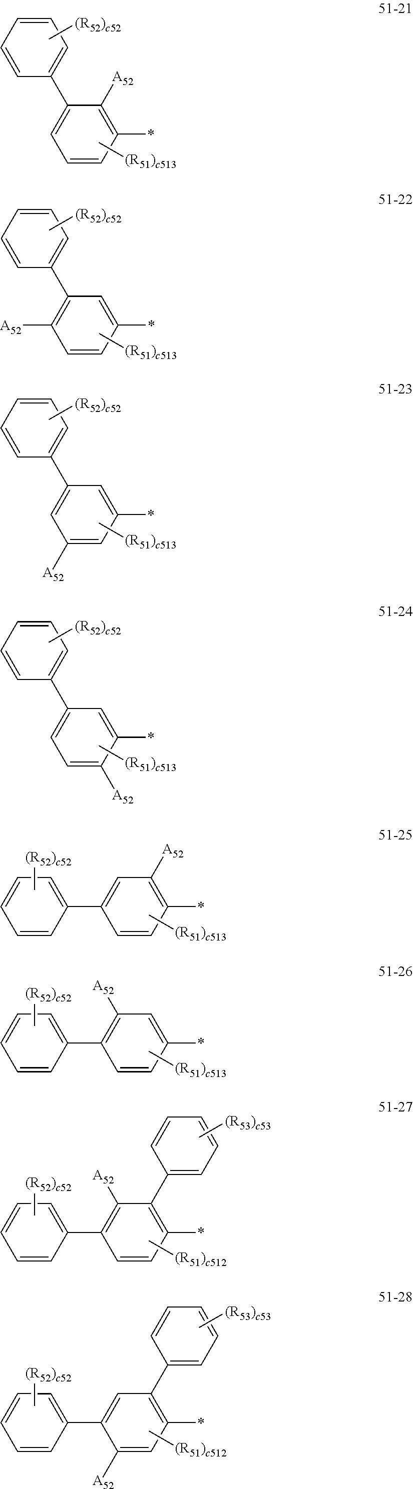 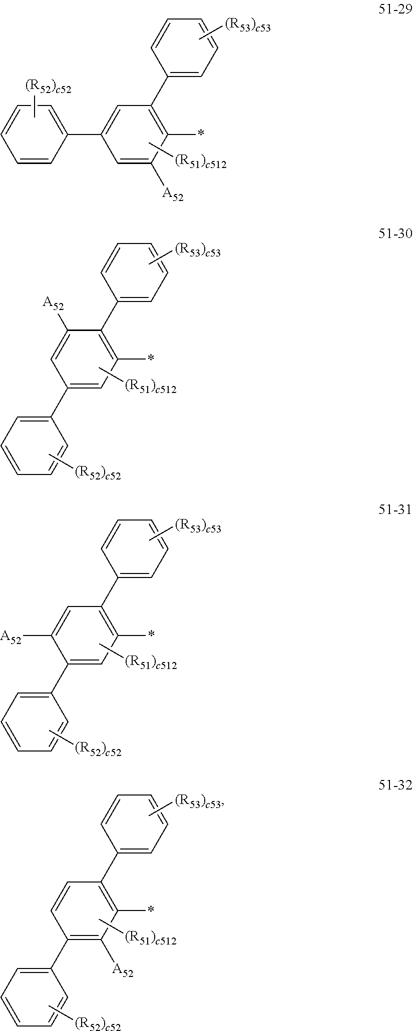
wherein, in Formulae 51-1 to 51-32,
R₅₁, R₅₂, c52, A₅₁, and A₅₂ are each independently the same as described in claim 1,
R₅₃ and c53 are each independently the same as described in connection with
R₅₂ and c52 in claim 1,
c512 is an integer of 0 to 2,
c513 is an integer of 0 to 3, and
indicates a binding site to L₅₁.

14. The organometallic compound of claim 1, wherein
the organometallic compound is represented by Formula 1-1 or 1-2: 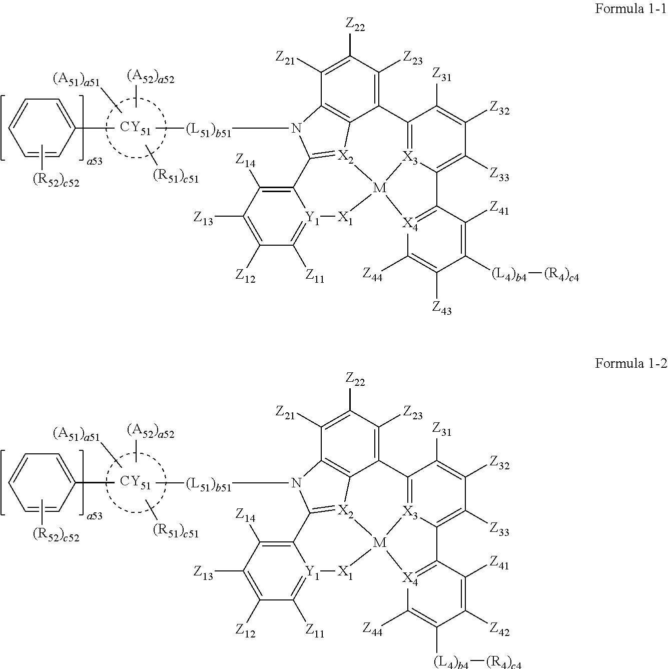
wherein, in Formulae 1-1 and 1-2
M, X₁ to X₄, Y₁, Y₃ to Y₅, L₄, L₅₁, b4, b51, R₅₁, R₅₂, c51, c52, A₅₁, A₅₂, a51, a52 and a53 are each independently the same as described in claim 1,
Z₁₁ to Z₁₄ are each independently the same as described in connection with R in claim 1,
Z₂₁ to Z₂₃ are each independently the same as described in connection with R₂ in claim 1,
Z₃₁ to Z₃₃ are each independently the same as described in connection with R₃ in claim 1,
Z₄₁ to Z₄₄ are each independently the same as described in connection with R₄ in claim 1,
two or more groups of Z₁₁ to Z₁₄ are optionally linked to form a C₅-C₃₀ carbocyclic group that is unsubstituted or substituted with an R₁₀ₐ group or a C₂-C₃₀ heterocyclic group that is unsubstituted or substituted with an R₁₀ₐ group,
two or more groups of Z₂₁ to Z₂₃ are optionally linked to form a C₅-C₃₀ carbocyclic group that is unsubstituted or substituted with an R₁₀ₐ or a C₂-C₃₀ heterocyclic group that is unsubstituted or substituted with an R₁₀ₐ group,
two or more groups of Z₃₁ to Z₃₃ are optionally linked to form a C₅-C₃₀ carbocyclic group that is unsubstituted or substituted with an R₁₀ₐ group or a C₂-C₃₀ heterocyclic group that is unsubstituted or substituted with an R₁₀ₐ group,
two or more groups of Z₄₁ to Z₄₄ are optionally linked to form a C₅-C₃₀ carbocyclic group that is unsubstituted or substituted with an R₁₀ₐ group or a C₂-C₃₀ heterocyclic group that is unsubstituted or substituted with an R₁₀ₐ group, and
R₁₀ₐ is the same as described in connection with R₁.

15. The organometallic compound of claim 14, wherein
Z₁₂, Z₁₄, Z₂₁ to Z₂₃, Z₃₁, Z₃₃ and Z₄₁ to Z₄₄ are each independently hydrogen, deuterium, —CH₃, or —CD₃.

16. An organic light-emitting device comprising:
a first electrode;
a second electrode; and
an organic layer disposed between the first electrode and the second electrode, wherein the organic layer comprises an emission layer, and an organometallic compound of claim 1.

17. The organic light-emitting device of claim 16, wherein
the first electrode is an anode,
the second electrode is a cathode,
the organic layer further comprises a hole transport region disposed between the first electrode and the emission layer and an electron transport region disposed between the emission layer and the second electrode,
wherein the hole transport region comprises a hole injection layer, a hole transport layer, an electron blocking layer, or any combination thereof, and
wherein the electron transport region comprises a hole blocking layer, an electron transport layer, an electron injection layer, or any combination thereof.

18. The organic light-emitting device of claim 16, wherein
the emission layer comprises the organometallic compound.

19. The organic light-emitting device of claim 18,
wherein the emission layer further comprises a host, and an amount of the host is larger than an amount of the organometallic compound.

20. A diagnostic composition comprising an organometallic compound of claim 1.
